(19) United States
(12) Patent Application Publication
(10) Pub. No.: US 2025/0304571 A1
(43) Pub. Date: Oct. 2, 2025

(54) BIOLOGICALLY ACTIVE COMPOUNDS AND METHODS THEREOF

(71) Applicants: Suzhou Keen Therapeutics Co., Ltd., Suzhou (CN); Shanghai Keen Therapeutics Co., Ltd., Shanghai (CN)

(72) Inventors: Yikai Wang, Shanghai (CN); Jun Yu, Shanghai (CN); Shien Liu, Shanghai (CN); Weihua Shi, Shanghai (CN); Chong Lu, Shanghai (CN)

(73) Assignees: Suzhou Keen Therapeutics Co., Ltd., Suzhou (CN); Shanghai Keen Therapeutics Co., Ltd., Shanghai (CN)

(21) Appl. No.: 18/874,206

(22) PCT Filed: Jun. 14, 2023
(86) PCT No.: PCT/CN2023/100170

§ 371 Date: Dec. 12, 2024

(30) Foreign Application Priority Data
Jun. 14, 2022 (WO) PCT/CN2022/098752

(51) Int. Cl. C07D 417/12; A61K 31/437; A61K 31/472; A61K 31/496; A61K 31/499; A61K 31/50; A61K 31/506; A61K 31/519; A61K 31/53; A61K 31/5355; A61K 31/5377; A61K 31/5383; A61K 31/5386; A61K 31/541; A61P 35/00; C07D 213/85; C07D 217/06; C07D 237/20; C07D 239/42; C07D 239/47; C07D 251/18; C07D 251/30; C07D 401/04; C07D 401/12; C07D 403/12; C07D 405/12; C07D 405/14; C07D 413/12; C07D 413/14; C07D 471/04; C07D 487/04; C07D 491/107; C07D 491/20; C07D 495/04; C07D 498/04; C07D 519/00

(52) U.S. Cl. CPC C07D 417/12; A61K 31/437; A61K 31/472; A61K 31/496; A61K 31/499; A61K 31/50; A61K 31/506; A61K 31/519; A61K 31/53; A61K 31/5355; A61K 31/5377; A61K 31/5383; A61K 31/5386; A61K 31/541; A61P 35/00; C07D 213/85; C07D 217/06; C07D 237/20; C07D 239/42; C07D 239/47; C07D 251/18; C07D 251/30; C07D 401/04; C07D 401/12; C07D 403/12; C07D 405/12; C07D 405/14; C07D 413/12; C07D 413/14; C07D 471/04; C07D 487/04; C07D 491/107; C07D 491/20; C07D 495/04; C07D 498/04; C07D 519/00

(57) ABSTRACT

Provided are compounds, compositions, methods, use for preventing or treating various conditions, disorders or diseases. In some embodiments, a condition, disorder or disease is cancer.

### TECHNICAL FIELD

Among other things, the present disclosure provides technologies, e.g., compounds, compositions, methods, etc. that are useful, e.g., for treating various conditions, disorders or diseases.

### BACKGROUND

Compounds have been reported to be useful for many applications, including for treating various conditions, disorders or diseases such as cancer. In some embodiments, ferroptosis dysfunction has been reported in many types of cancer.

### SUMMARY

Among other things, the present disclosure provides various compounds comprising an isourea or isothiourea moiety. In some embodiments, provided compounds comprise -T-C(—N═)[—N(—)—], wherein T is 0 or S. In some embodiments, an isourea moiety has the structure of —O—C(═NR^{w1})N(R^{w2})(R^{w3}) or a salt form thereof, wherein each variable is independently as described herein. In some embodiments, an isothiourea moiety has the structure of —S—C(═NR^{w1})N(R^{w2})(R^{w2}) or a salt form thereof, wherein each variable is independently as described herein.

In some embodiments, the present disclosure provides a compound comprising R^{w}, wherein R^{w} is -T-C(═NR^{w1})N(R^{w2})(R^{w3}) or a salt form thereof, wherein: T is O or S;
each of R^{w1}, R^{w2} and R^{w3} is independently -L-R′ or a detectable label, wherein:
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R′ is independently R, —OR, —C(O)R, —C(O)OR, —C(O)N(R)₂, —S(O)R, or —S(O)OR; and each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, the present disclosure provides a compound comprising -L^{w}-R^{w} or a salt form thereof, wherein L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—; and each other variable is independently as described herein.

For example, in some embodiments, the present disclosure provides a compound having the structure of formula A:

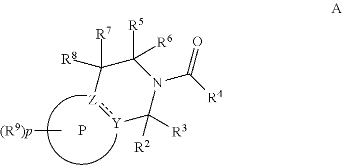

or a salt thereof, wherein:
Ring P is an optionally substituted 3-20 membered, monocyclic, bicyclic or polycyclic ring having 0-5 heteroatoms;
each of Y and Z is independently C or N;
each of R⁷ and R⁸ is independently R″;
each of R² and R³ is independently R″ or

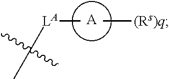

Ring A is an optionally substituted, 3-20 membered, monocyclic, bicyclic or polycyclic ring having 0-10 heteroatoms;
L^{A} is L;
R⁴ is or comprises an isourea or isothiourea moiety, or -L^{w}-R^{w};
L^{w} is a covalent bond, or an optionally substituted bivalent C₁₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
each of R⁵ and R⁶ is independently R″ or —C(O)OR″;
each of R⁹ and R^{s} is independently R″, halogen, —CN, oxo, —NO₂, or an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl;
each of p and q is independently 0-10;
each R″ is independently hydrogen, halogen, -L-R′, -L-OR′, -L-SR′, -L-C(O)OR′, -L-C(O)SR′, -L-C(O)N(R′)₂, -L-OC(O)N(R′)₂, -L-C(O)R′, -L-N(R′)₂, —CN, —OC(R′)₂COOH, —SC(R′)₂COOH, —N(R′)C(R′)₂COOH, or is an optionally substituted group selected from alkylsulfonyl, arylsulfonyl, carboxylate, ester, ether, amide, carbohydrate, amino acid, acyl, alkyloxy-substituted acyl, alditol, sulfate, sulfonamide, sulfoxide, sulfonate, sulfone, thioalkyl, thioester, and thioether;
each R′ is independently R, —OR, —C(O)R, —C(O)OR, —C(O)N(R)₂, —S(O)R, or —S(O)OR;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, the present disclosure provides a compound having the structure of A′:

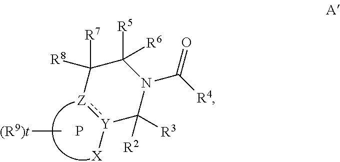

or a salt thereof, wherein:
X is —O—, —S—, —C(R′)=, —N(R′)—, or optionally substituted —CH═, —CH═CH—, or —NH—;
t is 0-9;
R¹ is R⁹; and
each other variable is independently as described herein.
   In some embodiments, a compound of formula A is a compound of formula A′.

In some embodiments, the present disclosure provides a compound having the structure of A-I:

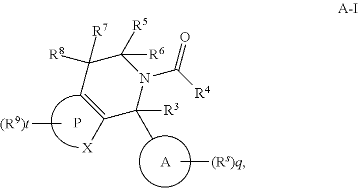

or a salt thereof, wherein each variable is independently as described herein. In some embodiments, a compound of formula A is a compound of formula A-I. In some embodiments, a compound of formula A′ is a compound of formula A-I.

In some embodiments, the present disclosure provides a compound having the structure of A-II:

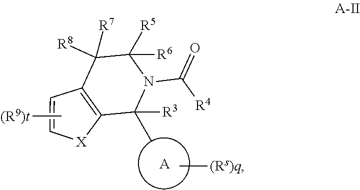

or a salt thereof, wherein each variable is independently as described herein. In some embodiments, a compound of formula A is a compound of formula A-II. In some embodiments, a compound of formula A′ is a compound of formula A-II. In some embodiments, a compound of formula A-I is a compound of formula A-II.

In some embodiments, the present disclosure provides a compound having the structure of A-III:

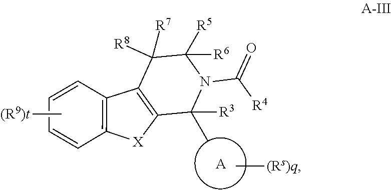

or a salt thereof, wherein each variable is independently as described herein. In some embodiments, a compound of formula A is a compound of formula A-III. In some embodiments, a compound of formula A′ is a compound of formula A-III. In some embodiments, a compound of formula A-I is a compound of formula A-III.

In some embodiments, the present disclosure provides a compound having the structure of formula B:

R^{b}-L^{b}-L^{R}-R⁴;

or a salt thereof, wherein:
L^{R} is

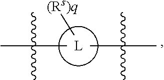

L, —C≡C—, optionally substituted —CH═CH—, —C(O)—, —C(S)—, or —C(NR″)—;
L^{b} is L;
R^{b} is R″;
Ring L is an optionally substituted, 3-20 membered, monocyclic, bicyclic or polycyclic ring having 0-10 heteroatoms;
R⁴ is or comprises an isourea or isothiourea moiety, or -L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
each of R⁵ is independently R″, halogen, —CN, oxo, —NO₂, or an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl;
q is 0-10;
each R″ is independently hydrogen, halogen, -L-R′, -L-OR′, -L-SR′, -L-C(O)OR′, -L-C(O)SR′, -L-C(O)N(R′)₂, -L-OC(O)N(R′)₂, -L-C(O)R′, -L-N(R′)₂, —CN, —OC(R′)₂COOH, —SC(R′)₂COOH, —N(R′)C(R′)₂COOH, or is an optionally substituted group selected from alkylsulfonyl, arylsulfonyl, carboxylate, ester, ether, amide, carbohydrate, amino acid, acyl, alkyloxy-substituted acyl, alditol, sulfate, sulfonamide, sulfoxide, sulfonate, sulfone, thioalkyl, thioester, and thioether;
each R′ is independently R, —OR, —C(O)R, —C(O)OR, —C(O)N(R)₂, —S(O)R, or —S(O)OR;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, the present disclosure provides a compound having the structure of B-1:

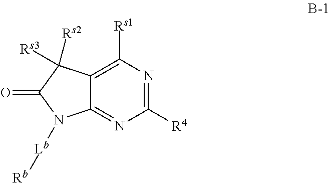

or a salt thereof, wherein:
each of R⁴, L^{b}, and R^{b} are as defined above and described herein; and
each of R^{s1}, R^{s2} and R^{s3} is independently R″, halogen, —CN, oxo, —NO₂, or an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl; and
   R′ is as defined above and described herein.

In some embodiments, L^{b} is -Cy- as described herein. In some embodiments, L^{b} is an optionally substituted phenyl ring. In some embodiments, L^{b} is an optionally substituted 5-6 membered heteroaryl ring having 1-4 heteroatoms independently selected from nitrogen, oxygen and sulfur. In some embodiments, L^{b} is an optionally substituted bicyclic 9-10 membered heteroaryl ring having 1-4 heteroatoms independently selected from nitrogen, oxygen and sulfur.

In some embodiments, the present disclosure provides a compound having the structure of B-1a, B-1b, B-1c, or B-1d:

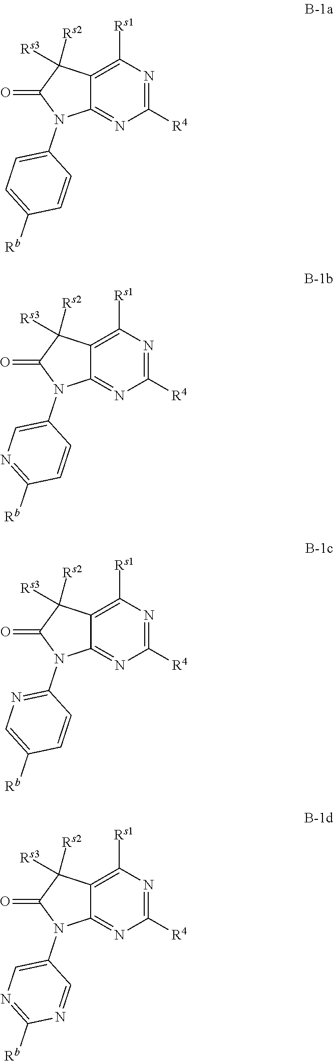

or a salt thereof, wherein:
each of R^{s1}, R^{s2}, R^{s3}, R⁴, and R^{b} are as defined above and described herein

In some embodiments, the present disclosure provides a compound having the structure of B-2a, B-2b, B-2c, B-2d or B-2e:

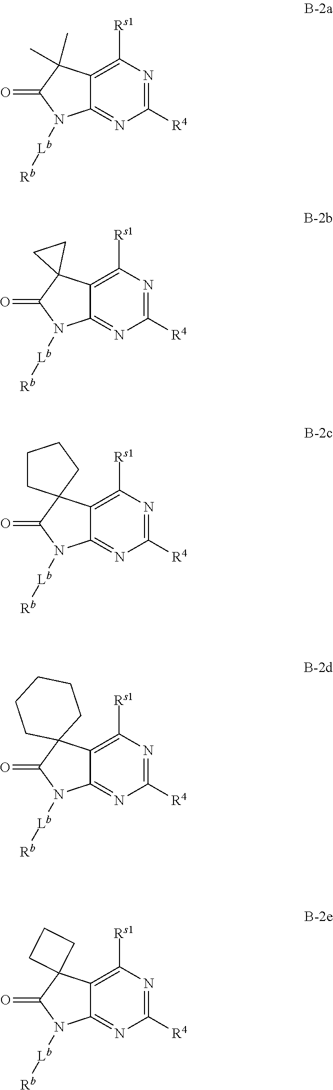

or a salt thereof, wherein:
each of R^{s1}, R⁴, L^{b}, and R^{b} is as defined above and described herein.

In some embodiments, the present disclosure provides a compound having the structure of B-3a, B-3b, B-3c, or B-3d:

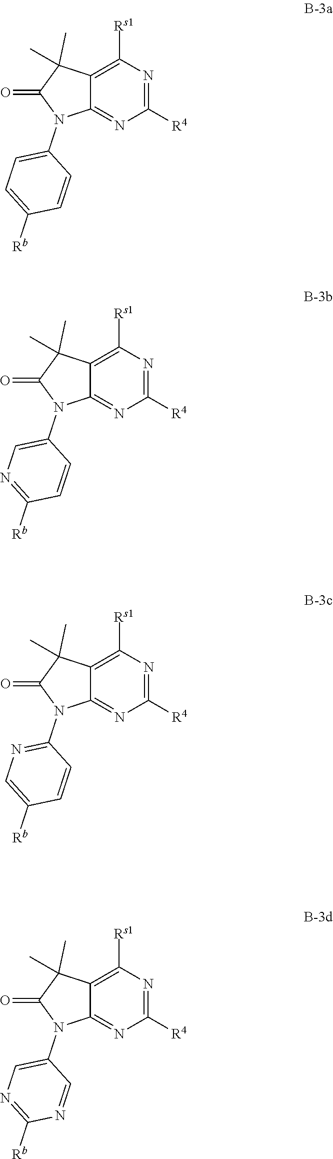

or a salt thereof, wherein:
each of R^{s1}, R⁴, and R^{b} is as defined above and described herein.

In some embodiments, the present disclosure provides a compound having the structure of B-4a, B-4b, B-4c, or B-4d:

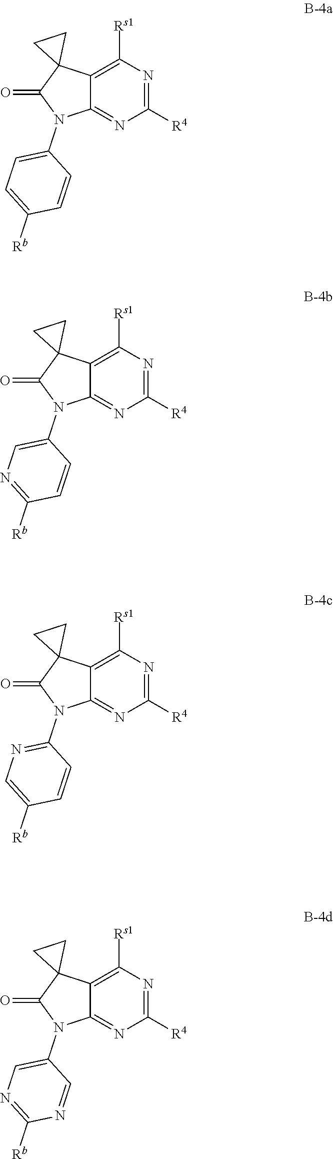

or a salt thereof, wherein:
each of R^{s1}, R⁴, and R^{b} is as defined above and described herein.

In some embodiments, the present disclosure provides a compound having the structure of B-5a, B-5b, B-5c, or B-5d:

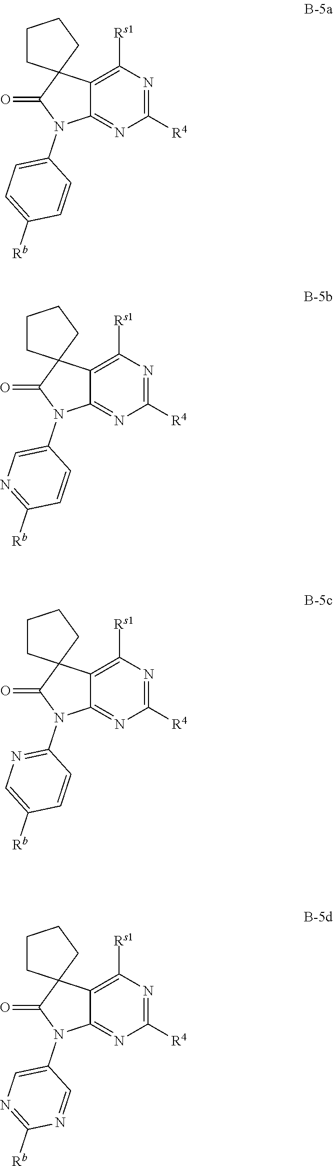

or a salt thereof, wherein:
each of R^{s1}, R⁴, and R^{b} is as defined above and described herein.

In some embodiments, the present disclosure provides a compound having the structure of B-6a, B-6b, B-6c, or B-6d:

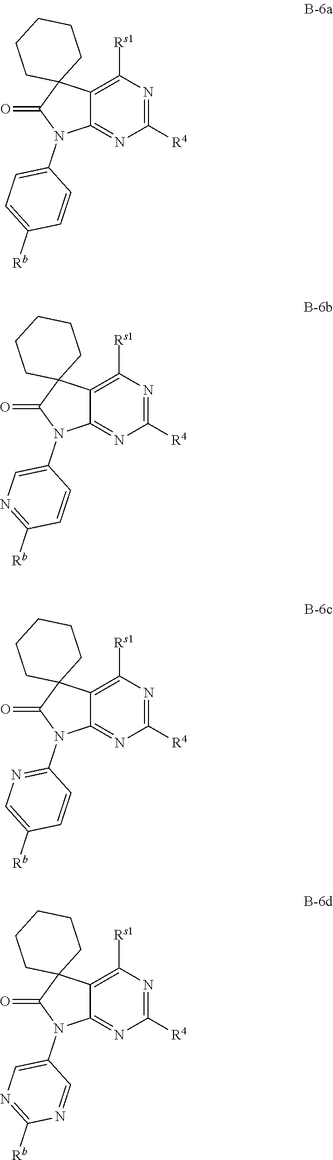

or a salt thereof, wherein:
each of R^{s1}, R⁴, and R^{b} is as defined above and described herein.

In some embodiments, the present disclosure provides a compound having the structure of B-7a, B-7b, B-7c, or B-7d:

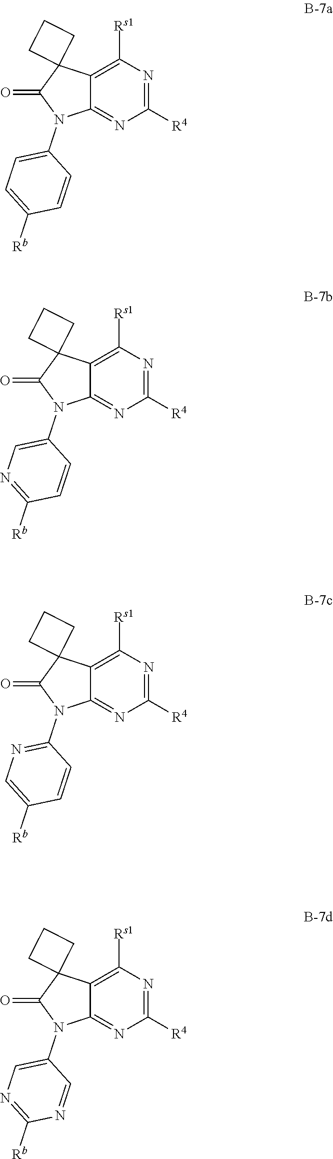

or a salt thereof, wherein:
each of R^{s1}, R⁴, and R^{b} is as defined above and described herein.

In some embodiments, the present disclosure provides a compound having the structure of formula C:

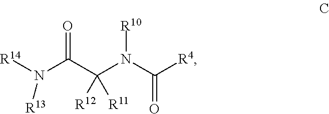

or a salt thereof, wherein:
R⁴ is or comprises an isourea or isothiourea moiety, or L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with C(R′)₂, -Cy-, —O—, —S—, N(R′), —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
each of R¹⁰, R¹¹, R¹², R¹³, and R¹⁴ is independently R″;
each R″ is independently hydrogen, halogen, -L-R′, -L-OR′, -L-SR′, -L-C(O)OR′, -L-C(O)SR′, -L-C(O)N(R′)₂, -L-OC(O)N(R′)₂, -L-C(O)R′, -L-N(R′)₂, —CN, —OC(R′)₂COOH, —SC(R′)₂COOH, or —N(R′)C(R′)₂COOH;
each R′ is independently R, —OR, —C(O)R, —C(O)OR, —C(O)N(R)₂, —S(O)R, or —S(O)OR;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R is independently halogen, —H, or an optionally substituted group selected from C₁₋₂₀ aliphatic, C₁₋₂₀ heteroaliphatic having 1-10 heteroatoms, C₆₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, the present disclosure provides a compound of formula D:

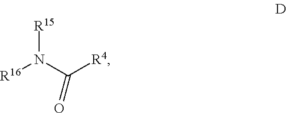

or a salt thereof, wherein:
each of R¹⁵ and R¹⁶ is independently -L-R′;
R⁴ is or comprises an isourea or isothiourea moiety, or -L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂, -Cy-, O, —S—, —N(R′)—, —C(O)—, —C(S)—, —C(NR′)—, —C(O)N(R′)—, C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NRW)N(R^{w2})(R^{w});
each of R^{w1}, R^{w2} and R^{w3} is independently -L-R′ or a detectable moiety;
T is O or S;
L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R′ is independently R, —OR, —C(O)R, —C(O)OR, —C(O)N(R)₂, —S(O)R, or —S(O)OR;
-Cy- is independently an optionally substituted, 3-20 membered, monocyclic, bicyclic or polycyclic ring having 0-10 heteroatoms;
each R is independently halogen, —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, the present disclosure provides a compound of formula E:

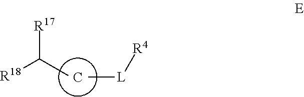

or a salt thereof, wherein:
R⁴ is or comprises an isourea or isothiourea moiety, or -L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
each of R¹⁷ and R¹⁸ is independently an optionally substituted 5-20 membered, monocyclic, bicyclic or polycyclic aromatic ring having 0-5 heteroatoms;
Ring C is an optionally substituted 3-20 membered, monocyclic, bicyclic or polycyclic ring having 0-6 heteroatoms.

L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R′ is independently R, —OR, —C(O)R, —C(O)OR, —C(O)N(R)₂, —S(O)R, or —S(O)OR;
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, the present disclosure provides a compound of formula F:

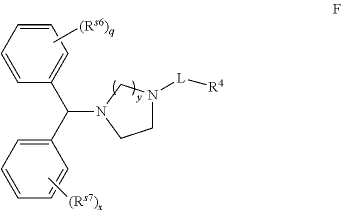

or a salt thereof, wherein:
R⁴ is or comprises an isourea or isothiourea moiety, or L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with C(R′)—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
each of R^{s6}, R^{s7} is independently R^{s};
each of R⁵ is independently R″, halogen, —CN, oxo, —NO₂, or an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl;
each R″ is independently hydrogen, halogen, -L-R′, -L-OR′, -L-SR′, -L-C(O)OR′, -L-C(O)SR′, -L-C(O)N(R′)₂, -L-OC(O)N(R′)₂, -L-C(O)R′, -L-N(R′)₂, —CN, —OC(R′)₂COOH, —SC(R′)₂COOH, —N(R′)C(R′)₂COOH, or is an optionally substituted group selected from alkylsulfonyl, arylsulfonyl, carboxylate, ester, ether, amide, carbohydrate, amino acid, acyl, alkyloxy-substituted acyl, alditol, sulfate, sulfonamide, sulfoxide, sulfonate, sulfone, thioalkyl, thioester, and thioether;
each of q, x and y is independently 0-4;
L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R′ is independently R, —OR, —C(O)R, —C(O)OR, —C(O)N(R)₂, —S(O)R, or —S(O)OR;
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, the present disclosure provides a compound of formula G:

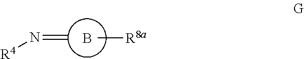

or a salt thereof, wherein:
Ring B is an optionally substituted, 3-20 membered, monocyclic, bicyclic or polycyclic ring having 0-10 heteroatoms;
R⁴ is or comprises an isourea or isothiourea moiety, or -L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
R^{8a} is R^{m}, or

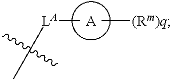

L^{A} is L;
each L is independently a covalent bond, or an optionally substituted bivalent C₁₋₁₀ to aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
Ring A is an optionally substituted, 3-20 membered, monocyclic, bicyclic or polycyclic ring having 0-10 heteroatoms;
each R^{m} is independently R^{s};
each of R⁵ is independently R″, halogen, —CN, oxo, —NO₂, or an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl;
each R″ is independently hydrogen, halogen, -L-R′, -L-OR′, -L-SR′, -L-C(O)OR′, -L-C(O)SR′, -L-C(O)N(R′)₂, -L-OC(O)N(R′)₂, -L-C(O)R′, -L-N(R′)₂, —CN, —OC(R′)₂COOH, —SC(R′)₂COOH, —N(R′)C(R′)₂COOH, or is an optionally substituted group selected from alkylsulfonyl, arylsulfonyl, carboxylate, ester, ether, amide, carbohydrate, amino acid, acyl, alkyloxy-substituted acyl, alditol, sulfate, sulfonamide, sulfoxide, sulfonate, sulfone, thioalkyl, thioester, and thioether;
q is independently 0-10;
each R′ is independently R, —OR, —C(O)R, —C(O)OR, —C(O)N(R)₂, —S(O)R, or —S(O)OR;
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, the present disclosure provides a compound of formula H:

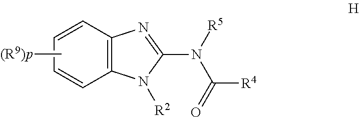

or a salt thereof, wherein:
R⁴ is or comprises an isourea or isothiourea moiety, or -L^{w}-R^{w};
L^{w} is a covalent bond, or an optionally substituted bivalent C₁₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
R⁵ is independently R″ or —C(O)OR″;
R² is independently R″ or

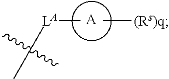

L^{A} is L;
Ring A is an optionally substituted, 3-20 membered, monocyclic, bicyclic or polycyclic ring having 0-10 heteroatoms;
each of R⁹ and R⁵ is independently R″, halogen, —CN, oxo, —NO₂, or an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl;
each of p and q is independently 0-10;
each R″ is independently hydrogen, halogen, -L-R′, -L-OR′, -L-SR′, -L-C(O)OR′, -L-C(O)SR′, -L-C(O)N(R′)₂, -L-OC(O)N(R′)₂, -L-C(O)R′, -L-N(R′)₂, —CN, —OC(R′)₂COOH, —SC(R′)₂COOH, —N(R′)C(R′)₂COOH, or is an optionally substituted group selected from alkylsulfonyl, arylsulfonyl, carboxylate, ester, ether, amide, carbohydrate, amino acid, acyl, alkyloxy-substituted acyl, alditol, sulfate, sulfonamide, sulfoxide, sulfonate, sulfone, thioalkyl, thioester, and thioether;
each R′ is independently R, —OR, —C(O)R, —C(O)OR, —C(O)N(R)₂, —S(O)R, or —S(O)OR;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or: two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, provided compounds are biological active and are useful for one or more properties and/or activities of various biological agents such polypeptides. In some embodiments, provided compounds are inhibitors. In some embodiments, provided compounds are covalent inhibitors. In some embodiments, an urea or isothiourea moiety is released after a compound comprising such a moiety is contacted with an agent comprising a nucleophile, e.g., —SH, —SeH, etc. In some embodiments, provided technologies, e.g., compounds, compositions, methods, etc., selectively react with certain nucleophile moieties over others. In some embodiments, provided technologies selectively react with —SeH nucleophiles over —SH nucleophiles. In some embodiments, provided technologies selectively react with selenocysteine residues over cysteine residues. In some embodiments, a selectivity is about or at least about 1.1, 1.2, 1.3, 1.4, 1.5, 1.6, 1.7, 1.8, 1.9, 2, 3, 4, 5, 6, 7, 8, 9, 10, 15, 20, 30, 40, 50, 60, 70, 80, 90, 100, 150, 200, 300, 400, 500, 1000 fold under comparable conditions.

In some embodiments, the present disclosure provides technologies for introducing a moiety into a system. In some embodiments, provided technologies comprise generating a urea from an isourea moiety, or a thiourea from an isothiourea moiety. In some embodiments, a compound having the structure of [LOAD]-R⁴ or a salt thereof, wherein [LOAD] is or comprises one or more small molecule, nucleic acid, polypeptide, lipid and/or carbohydrate moieties. In some embodiments, [LOAD] is attached to a nucleophilic moiety, e.g., —Se— or —S—. In some embodiments, [LOAD] is attached to a selenocysteine residue through —Se—. In some embodiments, [LOAD] is attached to a cysteine residue through —S—.

Provided technologies are useful for many applications. For example, in some embodiments, provided technologies are useful for modulating a properties and an activity of a polypeptide. In some embodiments, the present disclosure provides technologies for modulating a property of a polypeptide. I In some embodiments, the present disclosure provides technologies for modulating a property of a polypeptide in a system, comprising administering or delivering to the system a provided compound or composition. In some embodiments, the present disclosure provides technologies for inhibiting a property of a polypeptide. In some embodiments, a polypeptide comprises a nucleophilic moiety, e.g., —SeH, —SH, etc. In some embodiments, a polypeptide comprises a selenocysteine residue. In some embodiments, a polypeptide comprises a cysteine residue. In some embodiments, upon contact with a provided compound, a selenocysteine residue is covalently modified. In some embodiments, upon contact with a provided compound, a cysteine residue is covalently modified. In some embodiments, upon contact with a provided compound, a selenocysteine residue is covalently modified to a higher level compared to a cysteine residue in the same polypeptide. In some embodiments, a polypeptide comprises a characteristic sequence element of a GPX4. In some embodiments, a characteristic sequence element comprises an amino acid residue comprising —SeH. In some embodiments, a characteristic sequence element comprises selenocysteine. In some embodiments, a characteristic sequence element comprises an amino acid residue comprising —SH. In some embodiments, a characteristic sequence element comprises cysteine. In some embodiments, a polypeptide is GPX4. In some embodiments, a system an in vivo system. In some embodiments, a system an in vitro system. In some embodiments, a system is or comprises a cell. In some embodiments, a system is or comprises a tissue. In some embodiments, a system is or comprises an organ. In some embodiments, a system is a subject. In some embodiments, a system is an animal. In some embodiments, a system is a human. In some embodiments, an activity is modulated. In some embodiments, an activities is inhibited.

In some embodiments, provided technologies can inhibit cell proliferation. In some embodiments, provided technologies provide higher levels of ferroptosis compared to absence of the technologies or reference technologies. In some embodiments, provided technologies can induce or increase ferroptosis.

In some embodiments, provided compounds are useful for preventing or treating various conditions, disorders or diseases. In some embodiments, the present disclosure provides a method for preventing or treating a condition, disorder or disease, comprising administering or delivering to a subject susceptible thereto or suffering therefrom an effective amount of a provided compound or composition. In some embodiments, a condition, disorder or disease is a proliferative condition, disorder or disease. In some embodiments, a condition, disorder or disease is cancer. In some embodiments, a condition, disorder or disease is associated with GPX4. In some embodiments, a subject benefits from increased level of ferroptosis.

In some embodiments, the present disclosure provides a pharmaceutical composition comprising or delivering a provided compound and a pharmaceutically acceptable carrier.

Those skilled in the art reading the present disclosure will appreciate that isourea and/or isothiourea moieties described herein may be utilized for various purposes. For example, they can be utilized to replace leaving groups, e.g., halogen, in various agents for many applications, including replacing leaving groups in various biologically active compounds. In some embodiments, electrophilic moieties comprising isourea and/or isothiourea moieties, e.g., —C(O)—CH₂—R^{w} wherein the —CH₂— is optionally substituted, may be utilized to replace electrophilic groups, e.g., —C(O)—CH═CH₂, —C(O)—C≡CH, etc., in various agents for many applications, including replacing such groups in various biologically active compounds.

In some embodiments, the present disclosure provides technologies for manufacturing various compounds. In some embodiments, the present disclosure provides methods comprising reacting a first compound comprising a —OH group or a salt thereof with a second compound comprising —CN to form an isourea moiety. In some embodiments, the present disclosure provides methods comprising reacting a first compound comprising a —OH group or a salt thereof with a second compound comprising —N═C═N— to form an isourea moiety.

In some embodiments, the present disclosure provides technologies for assessing or characterizing various compounds and compositions. Many technologies, e.g., cells, animal models, clinical trials, etc., can be utilized in accordance with the present disclosure. Certain useful technologies are described in the Examples. In some embodiments, modifications of amino acid residues, e.g., selenocysteine and/or cysteine amino acid residues, may be assessed using mass spectrometry.

As appreciated by those skilled in the art, compounds of the present disclosure may be provided in various forms, e.g., salts, esters, solvates, prodrugs, etc. In some embodiments, a provided compound is in a salt form. In some embodiments, a provided compound is a pharmaceutically acceptable salt form. In some embodiments, a provided compound is in a solvate form. In some embodiments, a provided compound is a prodrug. In some embodiments, a provided compound is an ester. In some embodiments, a compounds is provided as a single stereoisomer. In some embodiments, a compounds is provided in a mixture or two or more stereoisomers.

### DETAILED DESCRIPTION OF CERTAIN EMBODIMENTS

Technologies of the present disclosure may be understood more readily by reference to the following detailed description of certain embodiments.

### Definitions

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001.

As used herein in the present disclosure, unless otherwise clear from context, (i) the term “a” or “an” may be understood to mean “at least one”; (ii) the term “or” may be understood to mean “and/or”; (iii) the terms “comprising”, “comprise”, “including” (whether used with “not limited to” or not), and “include” (whether used with “not limited to” or not) may be understood to encompass itemized components or steps whether presented by themselves or together with one or more additional components or steps; (iv) the term “another” may be understood to mean at least an additional/second one or more; (v) the terms “about” and “approximately” may be understood to permit standard variation as would be understood by those of ordinary skill in the art; and (vi) where ranges are provided, endpoints are included. Unless otherwise clear from context, isomers of compounds are included. As those skilled in the art, compounds may be provided, administered, or delivered in various forms, e.g., salts (e.g., pharmaceutically acceptable salts), solvates, hydrates, esters, prodrugs, tautomers, etc.

Aliphatic: As used herein, “aliphatic” means a straight-chain (i.e., unbranched) or branched, substituted or unsubstituted hydrocarbon chain that is completely saturated or that contains one or more units of unsaturation (but not aromatic), or a substituted or unsubstituted monocyclic, bicyclic, or polycyclic hydrocarbon ring that is completely saturated or that contains one or more units of unsaturation (but not aromatic), or combinations thereof. In some embodiments, aliphatic groups contain 1-50 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

Alkenyl: As used herein, the term “alkenyl” refers to an aliphatic group, as defined herein, having one or more double bonds.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkynyl: As used herein, the term “alkynyl” refers to an aliphatic group, as defined herein, having one or more triple bonds.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate and/or a pig). In some embodiments, animals include, but are not limited to, mammals, birds, reptiles, amphibians, fish and/or worms. In some embodiments, an animal may be a transgenic animal, a genetically-engineered animal and/or a clone.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, each monocyclic ring unit is aromatic. In some embodiments, an aryl group is a biaryl group. The term “aryl” may be used interchangeably with the term “aryl ring.” In certain embodiments of the present disclosure, “aryl” refers to an aromatic ring system which includes, but is not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

Characteristic portion: As used herein, the term “characteristic portion”, in the broadest sense, refers to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of amino acids, in some embodiments, a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

Comparable: The term “comparable” is used herein to describe two (or more) sets of conditions or circumstances that are sufficiently similar to one another to permit comparison of results obtained or phenomena observed. In some embodiments, comparable sets of conditions or circumstances are characterized by a plurality of substantially identical features and one or a small number of varied features. Those of ordinary skill in the art will appreciate that sets of conditions are comparable to one another when characterized by a sufficient number and type of substantially identical features to warrant a reasonable conclusion that differences in results obtained or phenomena observed under the different sets of conditions or circumstances are caused by or indicative of the variation in those features that are varied.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-C₁₀ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted forms thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar-”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, each monocyclic ring unit is aromatic. In some embodiments, a heteroaryl group has 6, 10, or 14 71 electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including oxidized forms of nitrogen, sulfur, phosphorus, or silicon; charged forms of nitrogen (e.g., quaternized forms, forms as in iminium groups, etc.), phosphorus, sulfur, oxygen; etc.). In some embodiments, a heteroatom is silicon, phosphorus, oxygen, sulfur or nitrogen. In some embodiments, a heteroatom is silicon, oxygen, sulfur or nitrogen. In some embodiments, a heteroatom is oxygen, sulfur or nitrogen.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Optionally Substituted: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. In some embodiments, an optionally substituted group is unsubstituted. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein. Certain substituents are described below.

Suitable monovalent substituents on a substitutable atom, e.g., a suitable carbon atom, are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR [symbol] , —(CH₂)₀₋₄CH(OR^{∘})₂; —(CH₂)₀₋₄Ph, which may be substituted with R^{∘}; —(CH₂)₀₋₄O(CH₂)₀₋₁Ph which may be substituted with R [symbol] —CH═CHPh, which may be substituted with R [symbol] , —(CH₂)₀₋₄O(CH₂)₀₋₁-pyridyl which may be substituted with R [symbol] , —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, —SC(S)SR^{∘}; —(CH₂)₀₋₄SC(O)R^{∘}; —(CH₂)₀₋₄C(O)NR^{∘}₂; —C(S)NR^{∘}₂; —C(S)SR [symbol] , —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —Si(R^{∘})₃; —OSi(R^{∘})₃; —B(R^{∘})₂; —OB(R^{∘})₂; —OB(OR^{∘})₂; —P(R^{∘})₂; —P(OR^{∘})₂; —P(R^{∘})(OR^{∘}); —OP(R^{∘})₂; —OP(OR^{∘})₂; —OP(R^{∘})(OR^{∘}); —P(O)(R^{∘})₂; —P(O)(OR^{∘})₂; —OP(O)(R^{∘})₂; —OP(O)(OR^{∘})₂; —OP(O)(OR^{∘})(SR^{∘}); —SP(O)(R^{∘})₂; —SP(O)(OR^{∘})₂; —N(R^{∘})P(O)(R^{∘})₂; —N(R^{∘})P(O)(OR^{∘})₂; —P(R^{∘})₂[B(R^{∘})₃]; —P(OR^{∘})₂[B(R^{∘})₃]; —OP(R^{∘})₂[B(R^{∘})₃]; —OP(OR^{∘})₂[B(R^{∘})₃]; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined herein and is independently hydrogen, C₁₋₂₀ aliphatic, C₁₋₂₀ heteroaliphatic having 1-5 heteroatoms independently selected from nitrogen, oxygen, sulfur, silicon and phosphorus, —CH₂—(C₆₋₁₄ aryl), —O(CH₂)₀₋₁(C₆₋₁₄ aryl), —CH₂-(5-14 membered heteroaryl ring), a 5-20 membered, monocyclic, bicyclic, or polycyclic, saturated, partially unsaturated or aryl ring having 0-5 heteroatoms independently selected from nitrogen, oxygen, sulfur, silicon and phosphorus, or, notwithstanding the definition above, two independent occurrences of R^{∘}, taken together with their intervening atom(s), form a 5-20 membered, monocyclic, bicyclic, or polycyclic, saturated, partially unsaturated or aryl ring having 0-5 heteroatoms independently selected from nitrogen, oxygen, sulfur, silicon and phosphorus, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, and a 5-6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents, e.g., on a suitable carbon atom, are independently the following: ═O, ═S, ═NNR*₂, =NNHC(O)R*, =NNHC(O)OR*, =NNHS(O)₂R*, =NR*, =NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, and an unsubstituted 5-6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, and an unsubstituted 5-6-membered saturated, partially unsaturated, and aryl ring having 0-4 heteroatoms independently selected from nitrogen, oxygen, and sulfur.

Suitable substituents on the aliphatic group of R* are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

In some embodiments, suitable substituents on a substitutable nitrogen are independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, and sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, and sulfur.

Suitable substituents on the aliphatic group of Rt are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, and sulfur.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Pharmaceutical composition: As used herein, the term “pharmaceutical composition” refers to an active agent, formulated together with one or more pharmaceutically acceptable carriers. In some embodiments, an active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises two or more acid groups. In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations.

Polypeptide: As used herein refers to a polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

Protecting group: The term “protecting group,” as used herein, is well known in the art and includes those described in detail in Protecting Groups in Organic Synthesis, T. W. Greene and P. G. M. Wuts, 3^{rd} edition, John Wiley & Sons, 1999, the entirety of which is incorporated herein by reference. Also included are those protecting groups specially adapted for nucleoside and nucleotide chemistry described in Current Protocols in Nucleic Acid Chemistry, edited by Serge L. Beaucage et al. 06/2012, the entirety of Chapter 2 is incorporated herein by reference. Suitable amino-protecting groups include methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, phenothiazinyl-(10)-carbonyl derivative, N′-p-toluenesulfonylaminocarbonyl derivative, N′-phenylaminothiocarbonyl derivative, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxycarbonylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, 2,4,6-trimethylbenzyl carbamate, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxycarbonylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, o-(benzoyloxymethyl)benzamide, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentacarbonylchromium- or tungsten)carbonyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, 3-nitropyridinesulfenamide (Npys), p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Suitably protected carboxylic acids further include, but are not limited to, silyl-, alkyl-, alkenyl-, aryl-, and arylalkyl-protected carboxylic acids. Examples of suitable silyl groups include trimethylsilyl, triethylsilyl, t-butyldimethylsilyl, t-butyldiphenylsilyl, triisopropylsilyl, and the like. Examples of suitable alkyl groups include methyl, benzyl, p-methoxybenzyl, 3,4-dimethoxybenzyl, trityl, t-butyl, tetrahydropyran-2-yl. Examples of suitable alkenyl groups include allyl. Examples of suitable aryl groups include optionally substituted phenyl, biphenyl, or naphthyl. Examples of suitable arylalkyl groups include optionally substituted benzyl (e.g., p-methoxybenzyl (MPM), 3,4-dimethoxybenzyl, O-nitrobenzyl, p-nitrobenzyl, p-halobenzyl, 2,6-dichlorobenzyl, p-cyanobenzyl), and 2- and 4-picolyl.

Suitable hydroxyl protecting groups include methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a, 4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1 methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl, p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, c-naphthyldiphenylmethyl, p-methoxyphenyldiphenylmethyl, di(p-methoxyphenyl)phenylmethyl, tri(p-methoxyphenyl)methyl, 4(4′ bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxycarbonyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts). For protecting 1,2- or 1,3-diols, the protecting groups include methylene acetal, ethylidene acetal, 1-t-butylethylidene ketal, 1-phenylethylidene ketal, (4-methoxyphenyl)ethylidene acetal, 2,2,2-trichloroethylidene acetal, acetonide, cyclopentylidene ketal, cyclohexylidene ketal, cycloheptylidene ketal, benzylidene acetal, p-methoxybenzylidene acetal, 2,4-dimethoxybenzylidene ketal, 3,4-dimethoxybenzylidene acetal, 2-nitrobenzylidene acetal, methoxymethylene acetal, ethoxymethylene acetal, dimethoxymethylene ortho ester, 1-methoxyethylidene ortho ester, 1-ethoxyethylidine ortho ester, 1,2-dimethoxyethylidene ortho ester, a-methoxybenzylidene ortho ester, 1-(N,N-dimethylamino)ethylidene derivative, a-(N,N′-dimethylamino)benzylidene derivative, 2-oxacyclopentylidene ortho ester, di-t-butylsilylene group (DTBS), 1,3-(1,1,3,3-tetraisopropyldisiloxanylidene) derivative (TIPDS), tetra-t-butoxydisiloxane-1,3-diylidene derivative (TBDS), cyclic carbonates, cyclic boronates, ethyl boronate, and phenyl boronate.

In some embodiments, a hydroxyl protecting group is acetyl, t-butyl, tbutoxymethyl, methoxymethyl, tetrahydropyranyl, 1-ethoxyethyl, 1-(2-chloroethoxy)ethyl, 2- trimethylsilylethyl, p-chlorophenyl, 2,4-dinitrophenyl, benzyl, benzoyl, p-phenylbenzoyl, 2,6-dichlorobenzyl, diphenylmethyl, p-nitrobenzyl, triphenylmethyl (trityl), 4,4′-dimethoxytrityl, trimethylsilyl, triethylsilyl, t-butyldimethylsilyl, t-butyldiphenylsilyl, triphenylsilyl, triisopropylsilyl, benzoylformate, chloroacetyl, trichloroacetyl, trifiuoroacetyl, pivaloyl, 9-fluorenylmethyl carbonate, mesylate, tosylate, triflate, trityl, monomethoxytrityl (MMTr), 4,4′-dimethoxytrityl, (DMTr) and 4,4′,4″-trimethoxytrityl (TMTr), 2-cyanoethyl (CE or Cne), 2-(trimethylsilyl)ethyl (TSE), 2-(2-nitrophenyl)ethyl, 2-(4-cyanophenyl)ethyl 2-(4-nitrophenyl)ethyl (NPE), 2-(4-nitrophenylsulfonyl)ethyl, 3,5-dichlorophenyl, 2,4-dimethylphenyl, 2-nitrophenyl, 4-nitrophenyl, 2,4,6-trimethylphenyl, 2-(2-nitrophenyl)ethyl, butylthiocarbonyl, 4,4′,4″-tris(benzoyloxy)trityl, diphenylcarbamoyl, levulinyl, 2-(dibromomethyl)benzoyl (Dbmb), 2-(isopropylthiomethoxymethyl)benzoyl (Ptmt), 9-phenylxanthen-9-yl (pixyl) or 9-(p-methoxyphenyl)xanthine-9-yl (MOX). In some embodiments, each of the hydroxyl protecting groups is, independently selected from acetyl, benzyl, t- butyldimethylsilyl, t-butyldiphenylsilyl and 4,4′-dimethoxytrityl. In some embodiments, the hydroxyl protecting group is selected from the group consisting of trityl, monomethoxytrityl and 4,4′-dimethoxytrityl group. In some embodiments, a protecting group is attached to a sulfur atom of an phosphorothioate group. In some embodiments, a protecting group is attached to an oxygen atom of an internucleotide phosphorothioate linkage. In some embodiments, a protecting group is attached to an oxygen atom of the internucleotide phosphate linkage. In some embodiments a protecting group is 2-cyanoethyl (CE or Cne), 2-trimethylsilylethyl, 2-nitroethyl, 2-sulfonylethyl, methyl, benzyl, o-nitrobenzyl, 2-(p-nitrophenyl)ethyl (NPE or Npe), 2-phenylethyl, 3-(N-tert-butylcarboxamido)-1-propyl, 4-oxopentyl, 4-methylthio-1-butyl, 2-cyano-1,1-dimethylethyl, 4-N-methylaminobutyl, 3-(2-pyridyl)-1-propyl, 2-[N-methyl-N-(2-pyridyl)]aminoethyl, 2-(N-formyl,N-methyl)aminoethyl, or 4-[N-methyl-N-(2,2,2-trifluoroacetyl)amino]butyl.

Subject: As used herein, the term “subject” or “test subject” refers to any organism to which a compound or composition is administered in accordance with the present disclosure e.g., for experimental, diagnostic, prophylactic and/or therapeutic purposes. Typical subjects include animals (e.g., mammals such as mice, rats, rabbits, non-human primates, and humans; insects; worms; etc.) and plants. In some embodiments, a subject is a human. In some embodiments, a subject may be suffering from and/or susceptible to a disease, disorder and/or condition.

Susceptible to: An individual who is “susceptible to” a disease, disorder and/or condition is one who has a higher risk of developing the disease, disorder and/or condition than does a member of the general public. In some embodiments, an individual who is susceptible to a disease, disorder and/or condition is predisposed to have that disease, disorder and/or condition. In some embodiments, an individual who is susceptible to a disease, disorder and/or condition may not have been diagnosed with the disease, disorder and/or condition. In some embodiments, an individual who is susceptible to a disease, disorder and/or condition may exhibit symptoms of the disease, disorder and/or condition. In some embodiments, an individual who is susceptible to a disease, disorder and/or condition may not exhibit symptoms of the disease, disorder and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the term “therapeutic agent” in general refers to any agent that elicits a desired effect (e.g., a desired biological, clinical, or pharmacological effect) when administered to a subject. In some embodiments, an agent is considered to be a therapeutic agent if it demonstrates a statistically significant effect across an appropriate population. In some embodiments, an appropriate population is a population of subjects suffering from and/or susceptible to a disease, disorder or condition. In some embodiments, an appropriate population is a population of model organisms. In some embodiments, an appropriate population may be defined by one or more criterion such as age group, gender, genetic background, preexisting clinical conditions, prior exposure to therapy. In some embodiments, a therapeutic agent is a substance that alleviates, ameliorates, relieves, inhibits, prevents, delays onset of, reduces severity of, and/or reduces incidence of one or more symptoms or features of a disease, disorder, and/or condition in a subject when administered to the subject in an effective amount. In some embodiments, a “therapeutic agent” is an agent that has been or is required to be approved by a government agency before it can be marketed for administration to humans. In some embodiments, a “therapeutic agent” is an agent for which a medical prescription is required for administration to humans. In some embodiments, a therapeutic agent is a provided compound.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

Unsaturated: The term “unsaturated,” as used herein, means that a moiety has one or more units of unsaturation.

As those skilled in the art will appreciate, methods and compositions described herein relating to provided compounds generally also apply to tautomers, pharmaceutically acceptable salts, solvates, etc. of such compounds. In some embodiments, a compound may be provided as a tautomer, salt, solvate, or a combination (e.g., a solvate of a salt) thereof.

### DESCRIPTION OF CERTAIN EMBODIMENTS

Among other things, the present disclosure provides compounds and compositions and methods thereof. In some embodiments, present disclosure provides technologies that are useful for preventing or treating various conditions, disorders or diseases. Certain embodiments of provided technologies are described below as examples.

### Certain Embodiments of Compounds

Among other things, the present disclosure provides compounds comprising an isourea moiety. In some embodiments, an isourea moiety is R^{w} as described herein wherein T is O. In some embodiments, the present disclosure provides compounds comprising an isothiourea moiety. In some embodiments, an isourea moiety is R^{w} as described herein wherein T is S. In some embodiments, the present disclosure provides urea compounds. In some embodiments, a urea compound has the structure of H—R^{w} or a tautomer and/or salt thereof, wherein T is O and each other variable is independently as described herein. In some embodiments, the present disclosure provides thiourea compounds. In some embodiments, a thiourea compound has the structure of H—R^{w} or a tautomer and/or salt thereof, wherein T is S and each other variable is independently as described herein. In some embodiments, a compound comprises or consists of two moieties, one of which is or comprises an isourea moiety, and the other is an moiety that can provide, facilitate or enhance interactions with a target, e.g., a polypeptide. In some embodiments, a compound comprises or consists of two moieties, one of which is or comprises an isothiourea moiety, and the other is an moiety that can provide, facilitate or enhance interactions with a target, e.g., a polypeptide. In some embodiments, a compound comprises or consists of two moieties, one of which is or comprises R^{w} or -L^{w}-R^{w}, and the other is an moiety that can provide, facilitate or enhance interactions with a target, e.g., a polypeptide. In some embodiments, isourea and isothiourea moieties may be utilized as leaving groups attached to electrophilic carbon atoms in warheads. In some embodiments, -L^{w}R^{w} described herein may be utilized as electrophilic warheads. In some embodiments, such warheads can be utilized to replace various electrophilic warhead moieties, e.g., —C(O)CH═CH₂, —C(O)C≡CH, —C(O)CH₂Cl, —C(O)CH₂Br, —C(O)CH₂I, etc. in various compounds to provide compounds useful for multiple purposes, e.g., as inhibitors for various polypeptides (e.g., kinases, proteases, etc.).

In some embodiments, an isourea moiety is R^{w}, wherein R^{w} is -T-C(═NR^{w1})N(R^{w2})(R^{w3}), T is —O—, and each other variable is independently as described herein. In some embodiments, an isothiourea moiety is R^{w}, wherein R^{w} is -T-C(═NR^{w1})N(R^{w2})(R^{w3}), T is —S—, and each other variable is independently as described herein.

In some embodiments, the present disclosure provides a compound comprising R^{w} as described herein. In some embodiments, the present disclosure provides a compound comprising -L^{w}-R^{w}, wherein each variable is independently as described herein. In some embodiments, the present disclosure provides a compound comprising —C(O)-L^{w}-R^{w}, wherein each variable is independently as described herein.

In some embodiments, R^{w} is —O—C(═NR^{w1})N(R^{w2})(R^{w3}), wherein each variable is independently as described herein. In some embodiments, R^{w} is —S—C(═NR^{w1})N(R^{w2})(R^{w3}), wherein each variable is independently as described herein.

Certain compounds are described below as examples. In some embodiments, the present disclosure provides a compound having the structure of formula A or a salt thereof, wherein each variable is independently as described herein. In some embodiments, the present disclosure provides a compound having the structure of formula B or a salt thereof, wherein each variable is independently as described herein. In some embodiments, the present disclosure provides a compound having the structure of formula C or a salt thereof, wherein each variable is independently as described herein. In some embodiments, the present disclosure provides a compound having the structure of formula D or a salt thereof, wherein each variable is independently as described herein. In some embodiments, the present disclosure provides a compound having the structure of formula E or a salt thereof, wherein each variable is independently as described herein. In some embodiments, the present disclosure provides a compound having the structure of formula F or a salt thereof, wherein each variable is independently as described herein. In some embodiments, the present disclosure provides a compound having the structure of formula G or a salt thereof, wherein each variable is independently as described herein. In some embodiments, the present disclosure provides a compound having the structure of formula H or a salt thereof, wherein each variable is independently as described herein. In some embodiments, the present disclosure provides a compound having the structure of [PAYLOAD]-R⁴ wherein each variable is independently as described herein. In some embodiments, the present disclosure provides a compound having the structure of N(CN)(R^{w2})(R^{w3}) wherein each variable is independently as described herein. In some embodiments, the present disclosure provides a compound having the structure of R^{w1}—N═C═N—R^{w3} or a salt thereof, wherein each variable is independently as described herein. In some embodiments, the present disclosure provides a compound having the structure of T=C(NHR^{w1})N(R^{w2})(R^{w3}) or a salt thereof, wherein each variable is independently as described herein.

Certain embodiments for various variables are described below as examples. Those skilled in the art reading the present disclosure will readily appreciate that embodiments for various variables can be combined in accordance with the present disclosure. Some combinations are described below as examples. In some embodiments, embodiments of a variable (e.g., R) are described when describing embodiments for other variables (e.g., various R embodiments are described when describing certain embodiments of R¹, R², etc.). Those skilled in the art reading the present disclosure readily appreciate that embodiments of a variable described when describing any one variable (e.g., R embodiments when describing R¹) may be applied to other variables that can be this variable (e.g., R², R³, etc. which can be R).

### L^{w}

In some embodiments, L^{w} is a covalent bond, or an optionally substituted bivalent C₁_₆(e.g., C₁, C₂, C₃, C₄, C₅ or C₆) aliphatic or heteroaliphatic having 1-6 (e.g., 1, 2, 3, 4, 5, or 6) heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{w} is a covalent bond, or an optionally substituted bivalent C₁₋₆ aliphatic, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{w} is a covalent bond, or an optionally substituted bivalent C₁₋₂ aliphatic, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—.

In some embodiments, as present in various compounds exemplified herein, L^{w} is or comprises optionally substituted —CH₂— bonded to R^{w}. In some embodiments, as present in various compounds exemplified herein, L^{w} is optionally substituted —CH₂—. In some embodiments, as present in various compounds exemplified herein, L^{w} is —CH₂—. In some embodiments, L^{w} is —CHD-. In some embodiments, L^{w} is —CD₂-. In some embodiments, L^{w} is mono-substituted —CH₂—. In some embodiments, L^{w} is mono-substituted —CD₂-. In some embodiments, such an optionally substituted —CH₂— is further activated through conjugation to an electron-withdrawing group, e.g., —C(O)— so that it is more electrophilic. In some embodiments, an electron-withdrawing group is or comprises —C(O)—. In some embodiments, an electron-withdrawing group is or comprises —C(S)—. In some embodiments, L^{w} is or comprises optionally substituted —C(O)—CH₂—. In some embodiments, L^{w} is —C(O)—CH₂—. In some embodiments, L^{w} is or comprises optionally substituted —C(S)—CH₂—. In some embodiments, L^{w} is —C(S)—CH₂—. In some embodiments, an electron-withdrawing group is or comprises —C(N(R′))—. In some embodiments, an electron-withdrawing group is or comprises an optionally substituted aromatic ring. In some embodiments, an electron-withdrawing group is or comprises an optionally substituted heteroaromatic ring. In some embodiments, an electron-withdrawing group is L^{R} as described herein. In some embodiments, L^{w} is —C(N(R′))—CH₂— wherein the —CH₂— is optionally substituted and R′ is as described herein. In some embodiments, L^{w} is optionally substituted —C(O)—CHD-. In some embodiments, L^{w} is optionally substituted —C(S)—CHD-. In some embodiments, L^{w} is —C(N(R′))—CHD- wherein the —CHD- is optionally substituted. In some embodiments, L^{w} is optionally substituted —C(O)—CD₂-. In some embodiments, L^{w} is optionally substituted —C(S)—CD₂-. In some embodiments, L^{w} is —C(N(R′))—CD₂- wherein the —CD₂- is optionally substituted. In some embodiments, the —CH₂—, —CHD-, or —CD₂- is not substituted. In some embodiments, the —CH₂—, —CHD-, or —CD₂- is bonded to R^{w}. In some embodiments, L^{w} is —CH(CH₃)—. In some embodiments, L^{w} is

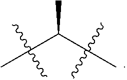

In some embodiments, L^{w} is

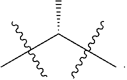

### R^{w}

As described herein, R^{w} is -TC(=NR^{w1})N(R^{w2})(R^{w3}) wherein each variable is independently as described herein. In some embodiments, R^{w} is —O—C(═NR^{w1})N(R^{w2})(R^{w3}) wherein each variable is independently as described herein. In some embodiments, R^{w} is —S—C(═NR^{w1})N(R^{w2})(R^{w3}) wherein each variable is independently as described herein.

In some embodiments, each of R^{w1}, R^{w2} and R^{w3} is independently R as described herein. In some embodiments, at least one of R^{w1}, R^{w2} and R^{w2} is —H. In some embodiments, one of R^{w1}, R^{w2} and R^{w2} is —H. In some embodiments, no more than one of R^{w1}, R^{w2} and R^{w2} is —H. In some embodiments, R^{w1} is —H. In some embodiments, R^{w3} is —H. In some embodiments, R^{w1} is —H. In some embodiments, none of R^{w1}, R^{w2} and R^{w2} is —H.

For example, in some embodiments, R^{w1} is —H, and R^{w2} and R^{w3} are each independently optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) aliphatic or are taken together with the nitrogen to which they are attached to form an optionally substituted ring as described herein. In some embodiments, R^{w1} is —H, and R^{w2} and R^{w3} are each independently optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) aliphatic or are taken together with the nitrogen to which they are attached to form an optionally substituted 3-10 (e.g., 3-7, 4-10, 5-10, 5-7, 3, 4, 5, 6, 7, 8, 9, or 10, etc.) membered ring having 0-3 (e.g., 0, 1-3, 1, 2, 3, etc.) heteroatoms in addition to the nitrogen atom. In some embodiments, R^{w1} is —H, and R^{w2} and R^{w3} are each independently optionally substituted C₁₋₁₀ aliphatic. In some embodiments, R^{w1} is —H, and R^{w2} and R^{w3} are each independently optionally substituted C₁₋₁₀ alkyl. In some embodiments, R^{w1} is —H, and R^{w2} and R^{w3} are taken together with the nitrogen to which they are attached to form an optionally substituted 3-10 (e.g., 3-7, 4-10, 5-10, 5-7, 3, 4, 5, 6, 7, 8, 9, or 10, etc.) membered ring having 0-3 (e.g., 0, 1-3, 1, 2, 3, etc.) heteroatoms in addition to the nitrogen atom. In some embodiments, R^{w1} is —H, and R^{w2} and R^{w3} are taken together with the nitrogen to which they are attached to form an optionally substituted 5-10 (e.g., 5-9, 5-7, 5, 6, 7, 8, 9, or 10, etc.) membered ring having 0-3 (e.g., 0, 1-3, 1, 2, 3, etc.) heteroatoms in addition to the nitrogen atom. In some embodiments, R^{w1} is —H, and R^{w2} and R^{w3} are taken together with the nitrogen to which they are attached to form an optionally substituted 5-10 (e.g., 5-9, 5-7, 5, 6, 7, 8, 9, or 10, etc.) membered ring having 0 heteroatom in addition to the nitrogen atom. In some embodiments, R^{w1} is —H, and R^{w2} and R^{w3} are taken together with the nitrogen to which they are attached to form an optionally substituted 5-10 (e.g., 5-9, 5-7, 5, 6, 7, 8, 9, or 10, etc.) membered ring having 1 heteroatom in addition to the nitrogen atom. In some embodiments, an additional heteroatom is nitrogen. In some embodiments, an additional heteroatom is oxygen.

In some embodiments, R^{w2} is —H, and R^{w1} and R^{w3} are each independently optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) aliphatic or are taken together with their intervening atoms to form an optionally substituted ring as described herein. In some embodiments, R^{w2} is —H, and R^{w1} and R^{w3} are each independently optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) aliphatic or are taken together with their intervening atoms to form an optionally substituted 3-10 (e.g., 3-7, 4-10, 5-10, 5-7, 3, 4, 5, 6, 7, 8, 9, or 10, etc.) membered ring having 0-3 (e.g., 0, 1-3, 1, 2, 3, etc.) heteroatoms in addition to the intervening atoms. In some embodiments, R^{w2} is —H, and R^{w1} and R^{w3} are each independently optionally substituted C₁₋₁₀ aliphatic. In some embodiments, R^{w2} is —H, and R^{w1} and R^{w3} are each independently optionally substituted C₁₋₁₀ alkyl. In some embodiments, R^{w2} is —H, and R^{w1} and R^{w3} are taken together with their intervening atoms to form an optionally substituted 3-10 (e.g., 3-7, 4-10, 5-10, 5-7, 3, 4, 5, 6, 7, 8, 9, or 10, etc.) membered ring having 0-3 (e.g., 0, 1-3, 1, 2, 3, etc.) heteroatoms in addition to the intervening atoms. In some embodiments, R^{w2} is —H, and R^{w1} and R^{w3} are taken together with their intervening atoms to form an optionally substituted 5-10 (e.g., 5-9, 5-7, 5, 6, 7, 8, 9, or 10, etc.) membered ring having 0-3 (e.g., 0, 1-3, 1, 2, 3, etc.) heteroatoms in addition to the intervening atoms. In some embodiments, R^{w2} is —H, and R^{w1} and R^{w3} are taken together with their intervening atoms to form an optionally substituted 5-10 (e.g., 5-9, 5-7, 5, 6, 7, 8, 9, or 10, etc.) membered ring having 0 heteroatom in addition to the intervening atoms. In some embodiments, R^{w2} is —H, and R^{w1} and R^{w3} are taken together with their intervening atoms to form an optionally substituted 5-10 (e.g., 5-9, 5-7, 5, 6, 7, 8, 9, or 10, etc.) membered ring having 1 heteroatom in addition to the intervening atoms. In some embodiments, an additional heteroatom is nitrogen. In some embodiments, an additional heteroatom is oxygen.

In some embodiments, a ring formed by R^{w2} and R^{w3} taken together with the nitrogen to which they are attached, or R^{w1} and one of R^{w2} and R^{w3} taken together with their intervening atoms, is 3-membered. In some embodiments, it is 4-membered. In some embodiments, it is 5-membered. In some embodiments, it is 6-membered. In some embodiments, it is 7-membered. In some embodiments, it is 8-membered. In some embodiments, it is 9-membered. In some embodiments, it is 10-membered. In some embodiments, a formed ring is saturated (e.g., when formed by R^{w2} and R^{w3} taken together with the nitrogen atom to which they are attached). In some embodiments, a formed ring is partially unsaturated. In some embodiments, a formed ring is aromatic. In some embodiments, a formed ring is substituted. In some embodiments, a formed ring is unsubstituted. In some embodiments, there is one heteroatom in the formed ring. In some embodiments, there are two heteroatoms in the formed ring. In some embodiments, there are three heteroatoms in the formed ring. In some embodiments, there are four heteroatoms in the formed ring. In some embodiments, a formed ring is monocyclic. In some embodiments, a formed ring is bicyclic. In some embodiments, a formed ring is polycyclic.

In some embodiments, —N(R^{w2})(R^{w3}) comprises one or more substituents, e.g., on R^{w2}, R^{w3}, a ring formed by R^{w2} and R^{w3} taken together with the nitrogen to which they are attached, a ring formed by R^{w1} and one of R^{w2} and R^{w3} taken together with their intervening atoms, etc. In some embodiments, —N(R^{w2})(R^{w3}) comprises one or more halogen substituents. In some embodiments, —N(R^{w2})(R^{w3}) comprises one or more (e.g., 1, 2, 3, etc.) —F. In some embodiments, a carbon atom at a beta position relative to the nitrogen to which R^{w2} and R^{w3} (there are two chemical bonds/one atom between the carbon atom and the nitrogen atom, e.g., beta carbon-alpha carbon-nitrogen) are attached are substituted. In some embodiments, it is substituted with —F. In some embodiments, it is mono-substituted and the substituent is —F. In some embodiments, it is substituted with two —F. In some embodiments, it is substituted with three —F. For example, in some embodiments, —N(R^{w2})(R^{w3}) is

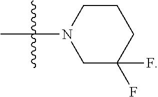 [SMILES: *N1CCCC(F)(F)C1]

In some embodiments, —N(R^{w2})(R^{w3}) is

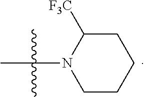 [SMILES: *N1CCCCC1C(F)(F)F]

In some embodiments, —N(R^{w2})(R^{w3}) is

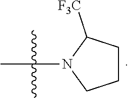 [SMILES: *N1CCCC1C(F)(F)F]

In some embodiments, —N(R^{w2})(R^{w3}) is

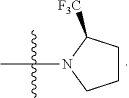 [SMILES: *N1CCC[C@@H]1C(F)(F)F]

In some embodiments, —N(R^{w2})(R^{w3}) is

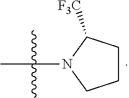 [SMILES: *N1CCC[C@H]1C(F)(F)F]

In some embodiments, —N(R^{w2})(R^{w3}) is

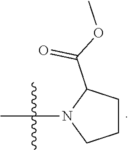 [SMILES: *N1CCCC1C(=O)OC]

In some embodiments, —N(R^{w2})(R^{w3}) is

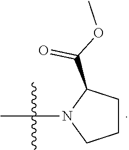 [SMILES: *N1CCC[C@@H]1C(=O)OC]

In some embodiments, —N(R^{w2})(R^{w3}) is

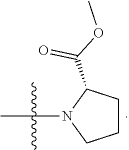 [SMILES: *N1CCC[C@H]1C(=O)OC]

In some embodiments, —N(R^{w2})(R^{w3}) is

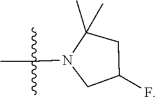 [SMILES: *N1CC(F)CC1(C)C]

In some embodiments, —N(R^{w2})(R^{w3}) is

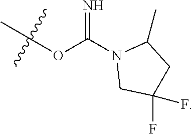 [SMILES: *OC(=N)N1CC(F)(F)CC1C]

In some embodiments, —N(R^{w2})(R^{w3}) is

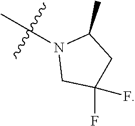 [SMILES: *N1CC(F)(F)C[C@@H]1C]

In some embodiments, —N(R^{w2})(R^{w3}) is

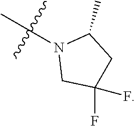 [SMILES: *N1CC(F)(F)C[C@H]1C]

In some embodiments, —N(R^{w2})(R^{w3}) is

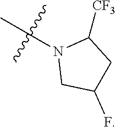 [SMILES: *N1CC(F)CC1C(F)(F)F]

In some embodiments, —N(R^{w2})(R^{w3}) is

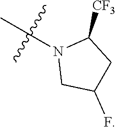 [SMILES: *N1CC(F)C[C@@H]1C(F)(F)F]

In some embodiments, —N(R^{w2})(R^{w3}) is

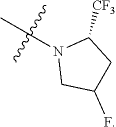 [SMILES: *N1CC(F)C[C@H]1C(F)(F)F]

In some embodiments, —N(R^{w2})(R^{w3}) is

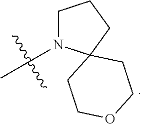 [SMILES: *N1CCCC12CCOCC2]

In some embodiments, —N(R^{w2})(R^{w3}) is

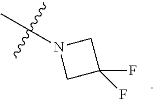 [SMILES: *N1CC(F)(F)C1]

In some embodiments, —N(R^{w2})(R^{w3}) is

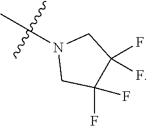 [SMILES: *N1CC(F)(F)C(F)(F)C1]

In some embodiments, —N(R^{w2})(R^{w3}) is

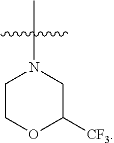 [SMILES: *N1CCOC(C(F)(F)F)C1]

In some embodiments, —N(R^{w2})(R^{w3}) is

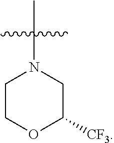 [SMILES: *N1CCO[C@@H](C(F)(F)F)C1]

In some embodiments, —N(R^{w2})(R^{w3}) is

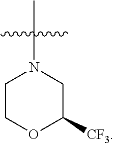 [SMILES: *N1CCO[C@H](C(F)(F)F)C1]

In some embodiments, —N(R^{w2})(R^{w3}) is

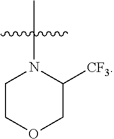 [SMILES: *N1CCOCC1C(F)(F)F]

In some embodiments, —N(R^{w2})(R^{w3}) is

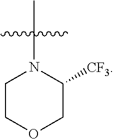 [SMILES: *N1CCOC[C@H]1C(F)(F)F]

In some embodiments, —N(R^{w2})(R^{w3}) is

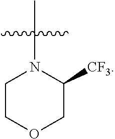 [SMILES: *N1CCOC[C@@H]1C(F)(F)F]

In some embodiments, —N(R^{w2})(R^{w3}) is

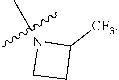

In some embodiments, —N(R^{w2})(R^{w3}) is

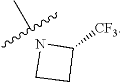

In some embodiments, —N(R^{w2})(R^{w3}) is

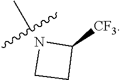

In some embodiments, R^{w2} is —CH₂CF₃. In some embodiments, a carbon atom at a gamma position relative to the nitrogen to which R^{w2} and R^{w3} (there are three chemical bonds/two atoms between the carbon atom and the nitrogen atom, e.g., gamma carbon-beta carbon-alpha carbon-nitrogen) are attached are substituted. In some embodiments, it is substituted with —F. In some embodiments, it is mono-substituted and the substituent is —F. In some embodiments, it is substituted with two —F. In some embodiments, it is substituted with three —F. For example, in some embodiments, —N(R^{w2})(R^{w3}) is

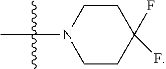 [SMILES: *N1CCC(F)(F)CC1]

In some embodiments, R^{w} is

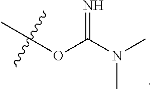 [SMILES: *OC(=N)N(C)C]

In some embodiments, R^{w} is

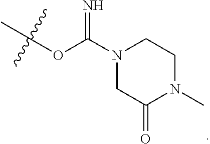 [SMILES: *OC(=N)N1CCN(C)C(=O)C1]

In some embodiments, R^{w} is

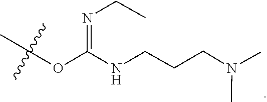 [SMILES: *O/C(=N/CC)NCCCN(C)C]

In some embodiments, R^{w} is

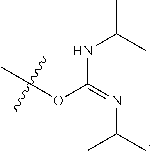 [SMILES: *O/C(=N\C(C)C)NC(C)C]

In some embodiments, R^{w} is

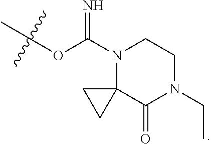 [SMILES: *OC(=N)N1CCN(CC)C(=O)C12CC2]

In some embodiments, R^{w} is

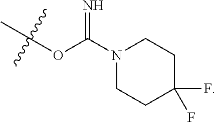 [SMILES: *OC(=N)N1CCC(F)(F)CC1]

In some embodiments, R^{w} is

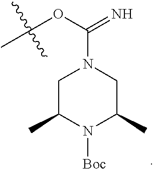 [SMILES: *OC(=N)N1C[C@@H](C)N(C(=O)OC(C)(C)C)[C@@H](C)C1]

In some embodiments, R^{w} is

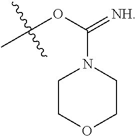 [SMILES: *OC(=N)N1CCOCC1]

In some embodiments, R^{w} is

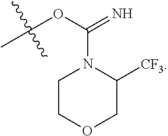 [SMILES: *OC(=N)N1CCOCC1C(F)(F)F]

In some embodiments, R^{w} is

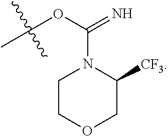 [SMILES: *OC(=N)N1CCOC[C@@H]1C(F)(F)F]

In some embodiments, R^{w} is

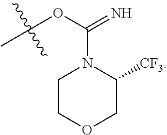 [SMILES: *OC(=N)N1CCOC[C@H]1C(F)(F)F]

In some embodiments, R^{w} is

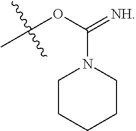 [SMILES: *OC(=N)N1CCCCC1]

In some embodiments, R^{w} is

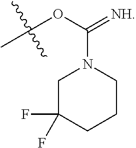 [SMILES: *OC(=N)N1CCCC(F)(F)C1]

In some embodiments, R^{w} is

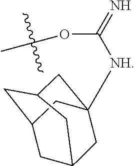 [SMILES: *OC(=N)NC12CC3CC(CC(C3)C1)C2]

In some embodiments, R^{w} is

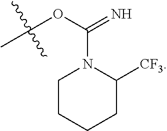 [SMILES: *OC(=N)N1CCCCC1C(F)(F)F]

In some embodiments, R^{w} is

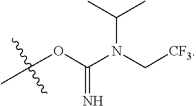 [SMILES: *OC(=N)N(CC(F)(F)F)C(C)C]

In some embodiments, R^{w} is

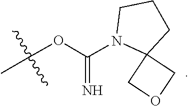 [SMILES: *OC(=N)N1CCCC12COC2]

In some embodiments, R^{w} is

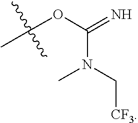 [SMILES: *OC(=N)N(C)CC(F)(F)F]

In some embodiments, R^{w} is

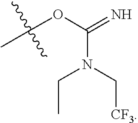 [SMILES: *OC(=N)N(CC)CC(F)(F)F]

In some embodiments, R^{w} is

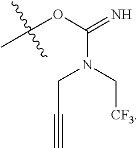 [SMILES: *OC(=N)N(CC#C)CC(F)(F)F]

In some embodiments, R^{w} is

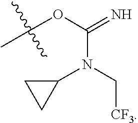 [SMILES: *OC(=N)N(CC(F)(F)F)C1CC1]

In some embodiments, R^{w} is

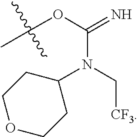 [SMILES: *OC(=N)N(CC(F)(F)F)C1CCOCC1]

In some embodiments, R^{w} is

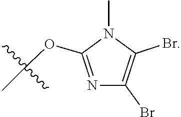 [SMILES: *Oc1nc(Br)c(Br)n1C]

In some embodiments, R^{w} is

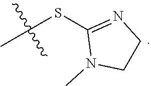 [SMILES: *SC1=NCCN1C]

In some embodiments, R^{w} is

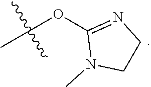 [SMILES: *OC1=NCCN1C]

In some embodiments, R^{w} is

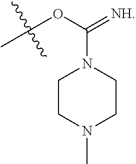 [SMILES: *OC(=N)N1CCN(C)CC1]

In some embodiments, R^{w} is

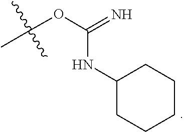 [SMILES: *OC(=N)NC1CCCCC1]

In some embodiments, R^{w} is

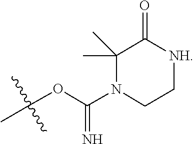 [SMILES: *OC(=N)N1CCNC(=O)C1(C)C]

In some embodiments, R^{w} is

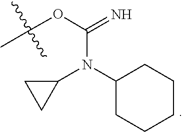 [SMILES: *OC(=N)N(C1CCCCC1)C1CC1]

In some embodiments, R^{w} is

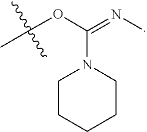

In some embodiments, R^{w} is

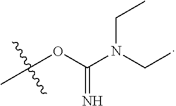

In some embodiments, R^{w} is

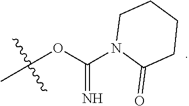 [SMILES: *OC(=N)N1CCCCC1=O]

In some embodiments, R^{w} is

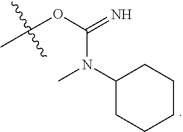 [SMILES: *OC(=N)N(C)C1CCCCC1]

In some embodiments, R^{w} is

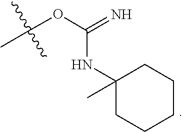 [SMILES: *OC(=N)NC1(C)CCCCC1]

In some embodiments, R^{w} is

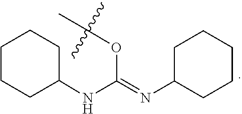 [SMILES: *O/C(=N\C1CCCCC1)NC1CCCCC1]

In some embodiments, R^{w} is

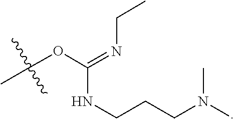

In some embodiments, R^{w} is

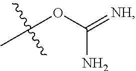 [SMILES: *OC(=N)N]

In some embodiments, R^{w} is

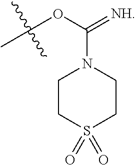 [SMILES: *OC(=N)N1CCS(=O)(=O)CC1]

In some embodiments, R^{w} is

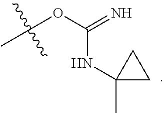 [SMILES: *OC(=N)NC1(C)CC1]

In some embodiments, R^{w} is

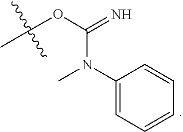 [SMILES: *OC(=N)N(C)c1ccccc1]

In some embodiments, R^{w} is

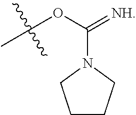 [SMILES: *OC(=N)N1CCCC1]

In some embodiments, R^{w} is

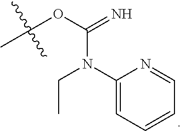 [SMILES: *OC(=N)N(CC)c1ccccn1]

In some embodiments, R^{w} is

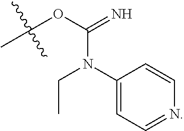 [SMILES: *OC(=N)N(CC)c1ccncc1]

In some embodiments, R^{w} is

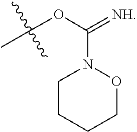 [SMILES: *OC(=N)N1CCCCO1]

In some embodiments, R^{w} is

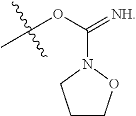 [SMILES: *OC(=N)N1CCCO1]

In some embodiments, R^{w} is

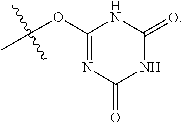 [SMILES: *Oc1nc(=O)[nH]c(=O)[nH]1]

In some embodiments, R^{w} is

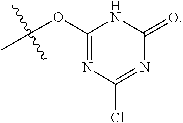 [SMILES: *Oc1nc(Cl)nc(=O)[nH]1]

In some embodiments, R^{w} is

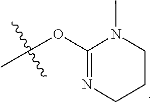 [SMILES: *OC1=NCCCN1C]

In some embodiments, R^{w} is

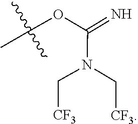 [SMILES: *OC(=N)N(CC(F)(F)F)CC(F)(F)F]

In some embodiments, R^{w} is

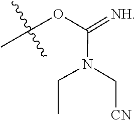 [SMILES: *OC(=N)N(CC)CC#N]

In some embodiments, R^{w} is

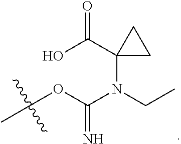 [SMILES: *OC(=N)N(CC)C1(C(=O)O)CC1]

In some embodiments, R^{w} is

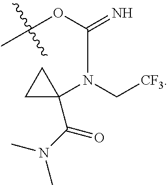 [SMILES: *OC(=N)N(CC(F)(F)F)C1(C(=O)N(C)C)CC1]

In some embodiments, R^{w} is

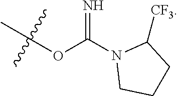 [SMILES: *OC(=N)N1CCCC1C(F)(F)F]

In some embodiments, R^{w} is

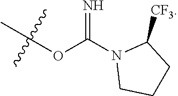 [SMILES: *OC(=N)N1CCC[C@@H]1C(F)(F)F]

In some embodiments, R^{w} is

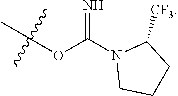 [SMILES: *OC(=N)N1CCC[C@H]1C(F)(F)F]

In some embodiments, R^{w} is

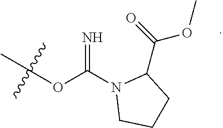 [SMILES: *OC(=N)N1CCCC1C(=O)OC ; "."]

In some embodiments, R^{w} is

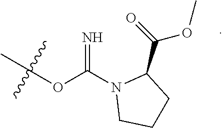 [SMILES: *OC(=N)N1CCC[C@@H]1C(=O)OC ; "."]

In some embodiments, R^{w} is

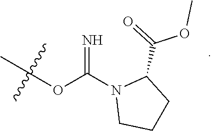 [SMILES: *OC(=N)N1CCC[C@H]1C(=O)OC ; "."]

In some embodiments, R^{w} is

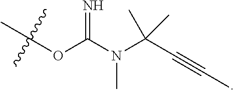

In some embodiments, R^{w} is

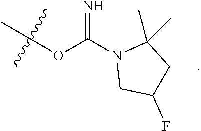 [SMILES: *OC(=N)N1CC(F)CC1(C)C ; "."]

In some embodiments, R^{w} is

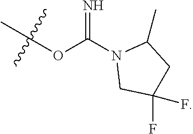 [SMILES: *OC(=N)N1CC(F)(F)CC1C]

In some embodiments, R^{w} is

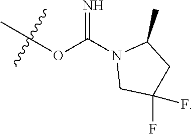 [SMILES: *OC(=N)N1CC(F)(F)C[C@@H]1C]

In some embodiments, R^{w} is

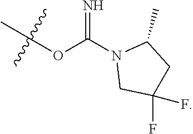 [SMILES: *OC(=N)N1CC(F)(F)C[C@H]1C]

In some embodiments, R^{w} is

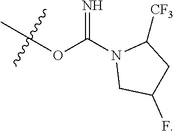 [SMILES: *OC(=N)N1CC(F)CC1C(F)(F)F]

In some embodiments, R^{w} is

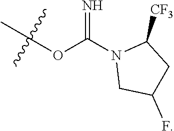 [SMILES: *OC(=N)N1CC(F)C[C@@H]1C(F)(F)F]

In some embodiments, R^{w} is

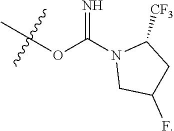 [SMILES: *OC(=N)N1CC(F)C[C@H]1C(F)(F)F]

In some embodiments, R^{w} is

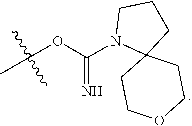 [SMILES: *OC(=N)N1CCCC12CCOCC2]

In some embodiments, R^{w} is

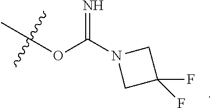 [SMILES: *OC(=N)N1CC(F)(F)C1]

In some embodiments, R^{w} is

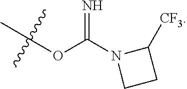 [SMILES: *OC(=N)N1CCC1C(F)(F)F]

In some embodiments, R^{w} is

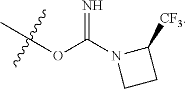 [SMILES: *OC(=N)N1CC[C@@H]1C(F)(F)F]

In some embodiments, R^{w} is

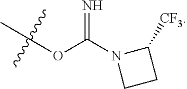 [SMILES: *OC(=N)N1CC[C@H]1C(F)(F)F]

In some embodiments, R^{w} is

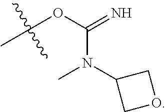 [SMILES: *OC(=N)N(C)C1COC1]

In some embodiments, R^{w} is

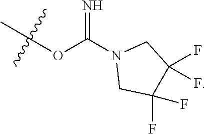 [SMILES: *OC(=N)N1CC(F)(F)C(F)(F)C1]

In some embodiments, R^{w} is

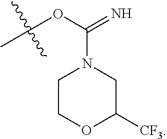 [SMILES: *OC(=N)N1CCOC(C(F)(F)F)C1]

In some embodiments, R^{w} is

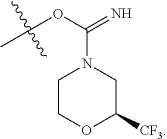 [SMILES: *OC(=N)N1CCO[C@H](C(F)(F)F)C1]

In some embodiments, R^{w} is

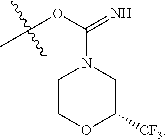 [SMILES: *OC(=N)N1CCO[C@@H](C(F)(F)F)C1]

In some embodiments, R^{w} is

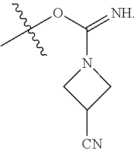 [SMILES: *OC(=N)N1CC(C#N)C1]

In some embodiments, R^{w} is

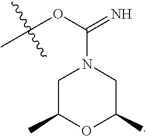

In some embodiments, R^{w} is

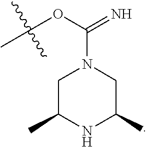

### T

In some embodiments, T is O. In some embodiments, T is S.

### R^{w1}

In some embodiments, R^{w4} is -L-R′ wherein each variable is independently as described herein. In some embodiments, R^{w1} is -L-R′ wherein the methylene unit of L that bonds to R′ is replaced with a moiety, e.g., —N(R′)— as described herein. In some embodiments, R^{w1} is R′ as described herein. In some embodiments, R^{w1} is R as described herein.

In some embodiments, R^{w1}is R as described herein. For example, in some embodiments, R^{w1} is —H. In some embodiments, R^{w1} is optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) aliphatic. In some embodiments, R^{w1} is optionally substituted C₁₋₁₀ alkyl. In some embodiments, R^{w1} is optionally substituted methyl. In some embodiments, R^{w1} is methyl. In some embodiments, R^{w1} is optionally substituted ethyl. In some embodiments, R^{w1} is ethyl. In some embodiments, R^{w1} is —CH₂CF₃. In some embodiments, R^{w1} is optionally substituted propyl. In some embodiments, R^{w1} is optionally substituted isopropyl. In some embodiments, R^{w1} is propyl. In some embodiments, R^{w1} is isopropyl. In some embodiments, R^{w1} is optionally substituted C₃₋₁₀ cycloaliphatic. In some embodiments, R^{w1} is optionally substituted C₃₋₁₀ cycloalkyl. In some embodiments, R^{w1} is optionally substituted cyclopropyl. In some embodiments, R^{w1} is cyclobutyl. In some embodiments, R^{w1} is cyclopentyl. In some embodiments, R^{w1} is cyclohexyl. In some embodiments, R^{w} is optionally substituted adamantyl. In some embodiments, R^{w1} is optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) heteroaliphatic having 1-6 (e.g., 1-3, 1-2, 1, 2, 3, 4, 5, 6, etc.) heteroatoms. In some embodiments, R^{w1} is optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) heteroaliphatic having 1, 2, or 3 heteroatoms. In some embodiments, R^{w1} is optionally substituted aryl. In some embodiments, R^{w} is optionally substituted 6-14 membered (e.g., 6, 10, 14, etc.) aryl. In some embodiments, R^{w1} is optionally substituted phenyl. In some embodiments, R^{w1} is optionally substituted 5-20 (e.g., 5-14, 5-10, 5-9, 5, 6, 8, 9, etc.) membered heteroaryl having 1-10 (e.g., 1-5, 1-4, 1-3, 1, 2, 3, 4, 5, 6, 7, 8, 9, 10, etc.) heteroatoms. In some embodiments, R^{w1} is optionally substituted 5-10 membered heteroaryl having 1-5 (e.g., 1-4, 1-3, 1, 2, 3, 4, 5, etc.) heteroatoms. In some embodiments, R^{w1} is optionally substituted 5-membered heteroaryl having 1-4 (e.g., 1-3, 1, 2, 3, 4, etc.) heteroatoms. In some embodiments, R^{w1} is optionally substituted 6-membered heteroaryl having 1-4 (e.g., 1-3, 1, 2, 3, 4, etc.) heteroatoms. In some embodiments, R^{w1} is optionally substituted 9-membered bicyclic heteroaryl having 1-4 (e.g., 1-3, 1, 2, 3, 4, etc.) heteroatoms. In some embodiments, R^{w1} is optionally substituted 3-20 (e.g., 3-15, 3-10, 3-9, 3-8, 3-7, 3-6, 3, 4, 5, 6, 7, 8, 9, 10, 11, 12, 13, 14, 15, 16, 17, 18, 19, 20, etc.) membered heterocyclyl having 1-10 (e.g., 1-5, 1-3, 1, 2, 3, 4, 5, 6, 7, 8, 9, 10, etc.) heteroatoms. In some embodiments, R^{w1} is optionally substituted 3-10 (e.g., 4-10, 5-10, 3, 4, 5, 6, 7, 8, 9, 10, etc.) membered heterocyclyl having 1-4 (e.g., 1-3, 1, 2, 3, 4, etc.) heteroatoms. In some embodiments, R^{w1} is optionally substituted 3-10 (e.g., 4-10, 5-10, 3, 4, 5, 6, 7, 8, 9, 10, etc.) membered saturated ring having 0-5 (e.g., 1-5, 1-4, 1-3, 0, 1, 2, 3, 4, 5, etc.) heteroatoms. In some embodiments, R^{w1} is optionally substituted 3-10 (e.g., 4-10, 5-10, 3, 4, 5, 6, 7, 8, 9, 10, etc.) membered partially saturated ring having 0-5 (e.g., 1-5, 1-4, 1-3, 0, 1, 2, 3, 4, 5, etc.) heteroatoms. In some embodiments, R^{w1} is an optionally substituted group which is a combination of two or more of C₁₋₂₀ aliphatic, C₁₋₂₀ heteroaliphatic having 1-10 heteroatoms, C₆₋₂₀ aryl, 5-20 membered heteroaryl having 1-10 heteroatoms, and 3-20 membered heterocyclyl having 1-10 heteroatoms as described herein, wherein the combination has 1-30 (e.g., 1-20, 1-15, 1-10, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, etc.) carbon atoms and 0-10 (e.g., 0-5, 1-10, 1-5, 0, 1, 2, 3, 4, 5, 6, 7, 8, 9, 10, etc.) heteroatoms. As used in the present disclosure, in some embodiments, each heteroatom is independently selected from nitrogen, oxygen and sulfur. In some embodiments, a heteroatom is nitrogen. In some embodiments, a heteroatom is oxygen. In some embodiments, a heteroatom is sulfur.

In some embodiments, R^{w1} is or comprises a detectable label, e.g., a fluorescent label, a radioactive label, an label useful in proteomics, an antibody label, a peptide label, etc.

In some embodiments, R^{w1} and R^{w2} are taken together with their intervening atoms to form an optionally substituted ring as described herein. In some embodiments, R^{w1} and R^{w2} are taken together with their intervening atoms to form an optionally substituted 3-10 (e.g., 3-7, 4-10, 5-10, 5-7, 3, 4, 5, 6, 7, 8, 9, or 10, etc.) membered ring having 0-3 (e.g., 0, 1-3, 1, 2, 3, etc.) heteroatoms in addition to their intervening atoms. In some embodiments, R^{w1} and R^{w2} are taken together with their intervening atoms to form an optionally substituted 3-10 (e.g., 3-7, 4-10, 5-10, 5-7, 3, 4, 5, 6, 7, 8, 9, or 10, etc.) membered ring having 0-3 (e.g., 0, 1-3, 1, 2, 3, etc.) heteroatoms in addition to the intervening atoms. In some embodiments, R^{w1} and R^{w2} are taken together with their intervening atoms to form an optionally substituted 5-14 (e.g., 5-10, 5, 6, 7, 8, 9, 10, 11, 12, 13, 14, etc.) membered ring having 0-6 (e.g., 1-6, 1-5, 1-4, 1-3, 0, 1, 2, 3, 4, 5, 6, etc.) heteroatoms in addition to the intervening atoms. In some embodiments, R^{w1} and R^{w2} are taken together with their intervening atoms to form an optionally substituted 5-10 (e.g., 5-9, 5-7, 5, 6, 7, 8, 9, or 10, etc.) membered ring having 0-3 (e.g., 0, 1-3, 1, 2, 3, etc.) heteroatoms in addition to the intervening atoms. In some embodiments, R^{w1} and R^{w2} are taken together with their intervening atoms to form an optionally substituted 5-10 (e.g., 5-9, 5-7, 5, 6, 7, 8, 9, or 10, etc.) membered ring having 0 heteroatom in addition to the intervening atoms. In some embodiments, R^{w1} and R^{w2} are taken together with their intervening atoms to form an optionally substituted 5-10 (e.g., 5-9, 5-7, 5, 6, 7, 8, 9, or 10, etc.) membered ring having 1 heteroatom in addition to the intervening atoms. In some embodiments, R^{w1} and R^{w3} are taken together with their intervening atoms to form an optionally substituted ring as described herein. In some embodiments, R^{w1} and R^{w3} are taken together with their intervening atoms to form an optionally substituted 3-10 (e.g., 3-7, 4-10, 5-10, 5-7, 3, 4, 5, 6, 7, 8, 9, or 10, etc.) membered ring having 0-3 (e.g., 0, 1-3, 1, 2, 3, etc.) heteroatoms in addition to their intervening atoms. In some embodiments, R^{w1} and R^{w3} are taken together with their intervening atoms to form an optionally substituted 3-10 (e.g., 3-7, 4-10, 5-10, 5-7, 3, 4, 5, 6, 7, 8, 9, or 10, etc.) membered ring having 0-3 (e.g., 0, 1-3, 1, 2, 3, etc.) heteroatoms in addition to the intervening atoms. In some embodiments, R^{w1} and R^{w3} are taken together with their intervening atoms to form an optionally substituted 5-14 (e.g., 5-10, 5, 6, 7, 8, 9, 10, 11, 12, 13, 14, etc.) membered ring having 0-6 (e.g., 1-6, 1-5, 1-4, 1-3, 0, 1, 2, 3, 4, 5, 6, etc.) heteroatoms in addition to the intervening atoms. In some embodiments, R^{w1} and R^{w3} are taken together with their intervening atoms to form an optionally substituted 5-10 (e.g., 5-9, 5-7, 5, 6, 7, 8, 9, or 10, etc.) membered ring having 0-3 (e.g., 0, 1-3, 1, 2, 3, etc.) heteroatoms in addition to the intervening atoms. In some embodiments, R^{w1} and R^{w3} are taken together with their intervening atoms to form an optionally substituted 5-10 (e.g., 5-9, 5-7, 5, 6, 7, 8, 9, or 10, etc.) membered ring having 0 heteroatom in addition to the intervening atoms. In some embodiments, R^{w1} and R^{w3} are taken together with their intervening atoms to form an optionally substituted 5-10 (e.g., 5-9, 5-7, 5, 6, 7, 8, 9, or 10, etc.) membered ring having 1 heteroatom in addition to the intervening atoms. In some embodiments, an additional heteroatom is nitrogen. In some embodiments, an additional heteroatom is oxygen. In some embodiments, a formed ring is 3-membered. In some embodiments, it is 4-membered. In some embodiments, it is 5-membered. In some embodiments, it is 6-membered. In some embodiments, it is 7-membered. In some embodiments, it is 8-membered. In some embodiments, it is 9-membered. In some embodiments, it is 10-membered. In some embodiments, a formed ring is substituted. In some embodiments, a formed ring is unsubstituted. In some embodiments, there is one heteroatom in the formed ring. In some embodiments, there are two heteroatoms in the formed ring. In some embodiments, there are three heteroatoms in the formed ring. In some embodiments, there are four heteroatoms in the formed ring. In some embodiments, a formed ring is monocyclic. In some embodiments, a formed ring is bicyclic. In some embodiments, a formed ring is polycyclic. In some embodiments, a formed ring is partially unsaturated. For example, in some embodiments, a formed ring is an optionally substituted 5-6 membered partially unsaturated ring having 1-2 (in some embodiments, 1; in some embodiments, 2) heteroatoms one or each of which is nitrogen. In some embodiments, a formed ring is an optionally substituted 5-membered partially unsaturated ring having 1-2 heteroatoms each of which is nitrogen. In some embodiments, a formed ring is aromatic. In some embodiments, a formed ring is optionally substituted 5-6 membered heteroaryl having 1-3 (e.g., 1-2, 1, 2, 3, etc.) heteroatoms one or more of which are nitrogen. In some embodiments, a formed ring is optionally substituted 5-membered heteroaryl having 1-3 heteroatoms one or more of which are nitrogen. In some embodiments, a formed ring is optionally substituted 5-membered heteroaryl having 1 or 2 nitrogen atoms. In some embodiments, a formed ring is an optionally substituted 6-membered partially unsaturated ring having 1, 2, or 3 heteroatoms independently selected from nitrogen, oxygen and sulfur. In some embodiments, a formed ring is an optionally substituted 6-membered partially unsaturated ring having 1, 2, or 3 nitrogen atoms. In some embodiments, a formed ring is optionally substituted 6-membered heteroaryl having 1, 2, or 3 heteroatoms independently selected from nitrogen, oxygen and sulfur. In some embodiments, a formed ring is optionally substituted 6-membered heteroaryl having 1, 2, or 3 nitrogen atoms.

For example, in some embodiments, a formed ring is optionally substituted

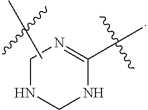

In some embodiments, —C(═NR^{w1})N(R^{w2}) is optionally substituted

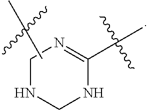

In some embodiments, —C(═NR^{w1})N(R^{w3}) is optionally substituted

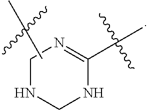

In some embodiments, a formed ring is optionally substituted

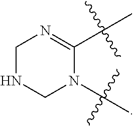

In some embodiments, —C(═NR^{w1})N(R^{w2}) is optionally substituted

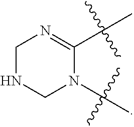

In some embodiments, —C(═NR^{w1})N(R^{w3}) is optionally substituted

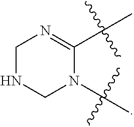

In some embodiments, a formed ring is optionally substituted

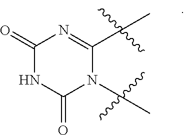 [SMILES: *c1nc(=O)[nH]c(=O)n1*]

In some embodiments, —C(═NR^{w1})N(R^{w2}) is optionally substituted

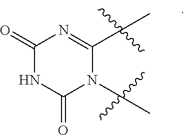 [SMILES: *c1nc(=O)[nH]c(=O)n1*]

In some embodiments, —C(═NR^{w1})N(R^{w3}) is optionally substituted

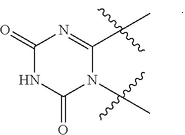 [SMILES: *c1nc(=O)[nH]c(=O)n1*]

In some embodiments, a formed ring is optionally substituted

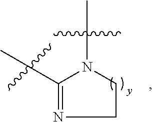

wherein y is independently 0, 1, 2, 3 or 4. In some embodiments, —C(═NR^{w1})N(R^{w2}) is optionally substituted

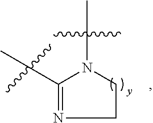

wherein y is independently 0, 1, 2, 3 or 4. In some embodiments, —C(═NR^{w1})N(R^{w3}) is optionally substituted

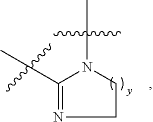

wherein y is independently 0, 1, 2, 3 or 4. In some embodiments, a formed ring is optionally substituted

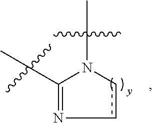

wherein y is independently 0, 1, 2, 3 or 4, and [symbol] is single bond or double bond. In some embodiments, —C(═NR^{w1})N(R^{w3}) is optionally substituted

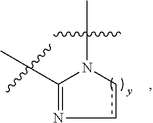

wherein y is independently 0, 1, 2, 3 or 4, and [symbol] is single bond or double bond. In some embodiments, —C(═NR^{w1})N(R^{w3}) is optionally substituted

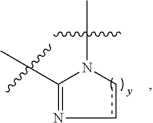

wherein y is independently 0, 1, 2, 3 or 4, and [symbol] is single bond or double bond. In some embodiments, y is 0. In some embodiments, y is 1. In some embodiments, y is 2. In some embodiments, y is 3. In some embodiments, y is 4. In some embodiments, a formed ring is optionally substituted

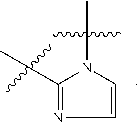 [SMILES: *c1nccn1*]

In some embodiments, —C(═NR^{w1})N(R^{w2}) is optionally substituted

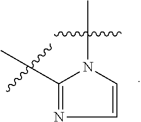 [SMILES: *c1nccn1*]

In some embodiments, —C(═N^{w1})N(R^{w3}) is optionally substituted

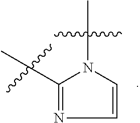 [SMILES: *c1nccn1*]

In some embodiments, —C(═NR^{w1})N(R^{w2})(R^{w3}) is optionally substituted

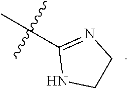 [SMILES: *C1=NCCN1]

In some embodiments, C(═NR^{w1})N(R^{w2})(R^{w3}) is optionally substituted

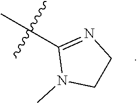 [SMILES: *C1=NCCN1C]

In some embodiments, —C(═NR^{w1})N(R^{w2})(R^{w3}) is optionally substituted

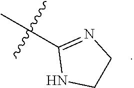 [SMILES: *C1=NCCN1]

In some embodiments, —C(═NR^{w3})N(R^{w2})(R^{w3}) is optionally substituted

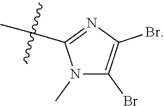 [SMILES: *c1nc(Br)c(Br)n1C]

In some embodiments, —C(═NR^{w1})N(R^{w2})(R^{w3}) is optionally substituted

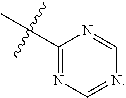 [SMILES: *c1ncncn1]

In some embodiments, —C(═NR^{w1})N(R^{w2})(R^{w3}) is optionally substituted

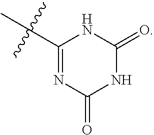 [SMILES: *c1nc(=O)[nH]c(=O)[nH]1]

In some embodiments, —C(═NR^{w1})N(R^{w2})(R^{w3}) is

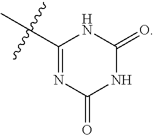 [SMILES: *c1nc(=O)[nH]c(=O)[nH]1]

In some embodiments, a formed ring is

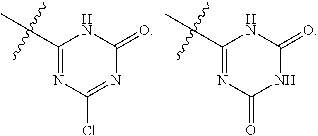 [SMILES: *c1nc(Cl)nc(=O)[nH]1 ; *c1nc(=O)[nH]c(=O)[nH]1]

In some embodiments, a formed ring is

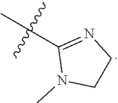 [SMILES: *C1=NCCN1C]

In some embodiments, a formed ring is

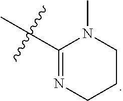 [SMILES: *C1=NCCCN1C]

### R^{w2}

In some embodiments, R^{w2} is -L-R′ wherein each variable is independently as described herein. In some embodiments, R^{w2} is -L-R′ wherein the methylene unit of L that bonds to R′ is replaced with a moiety, e.g., —N(R′)— as described herein. For example, in some embodiments, R^{w2} is —(CH₂)₃—N(CH₃)—CH₃. In some embodiments, R^{w2} is —C(CH₂)₂—C≡C—CH₃. In some embodiments, R^{w2} is —CH₂—CF₃. In some embodiments, R^{w2} is —CH₂—C═CH. In some embodiments, R^{w2} is R′ as described herein. In some embodiments, R^{w2} is R as described herein.

In some embodiments, R^{w2} is R as described herein. For example, in some embodiments, R^{w2} is —H. In some embodiments, R^{w2} is optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) aliphatic. In some embodiments, R^{w2} is optionally substituted C₁₋₁₀ alkyl. In some embodiments, R^{w2} is optionally substituted methyl. In some embodiments, R^{w} is methyl. In some embodiments, R^{w2} is optionally substituted ethyl. In some embodiments, R^{w2} is ethyl. In some embodiments, R^{w2} is —CH₂CF₃. In some embodiments, R^{w2} is optionally substituted propyl. In some embodiments, R^{w2} is optionally substituted isopropyl. In some embodiments, R^{w2} is propyl. In some embodiments, R^{w2} is isopropyl. In some embodiments, R^{w2} is optionally substituted C₁₆ haloalkyl. In some embodiments, R^{w2} is optionally substituted C₃₋₁₀ cycloaliphatic. In some embodiments, R^{w2} is optionally substituted C₃₋₁₀ cycloalkyl. In some embodiments, R^{w2} is optionally substituted cyclopropyl. In some embodiments, R^{w2} is cyclobutyl. In some embodiments, R^{w2} is cyclopentyl. In some embodiments, R^{w2} is cyclohexyl. In some embodiments, R^{w2} is optionally substituted adamantyl. In some embodiments, R^{w2} is adamantyl. In some embodiments, R^{w2} is 1-adamantyl. In some embodiments, R^{w2} is optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) heteroaliphatic having 1-6 (e.g., 1-3, 1-2, 1, 2, 3, 4, 5, 6, etc.) heteroatoms. In some embodiments, R^{w2} is optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) heteroaliphatic having 1, 2, or 3 heteroatoms. In some embodiments, R^{w2} is optionally substituted oxetanyl. In some embodiments, R^{w2} is optionally substituted aryl. In some embodiments, R^{w2} is optionally substituted 6-14 membered (e.g., 6, 10, 14, etc.) aryl. In some embodiments, R^{w2} is optionally substituted phenyl. In some embodiments, R^{w2} is optionally substituted 5-20 (e.g., 5-14, 5-10, 5-9, 5, 6, 8, 9, etc.) membered heteroaryl having 1-10 (e.g., 1-5, 1-4, 1-3, 1, 2, 3, 4, 5, 6, 7, 8, 9, 10, etc.) heteroatoms. In some embodiments, R^{w2} is optionally substituted 5-10 membered heteroaryl having 1-5 (e.g., 1-4, 1-3, 1, 2, 3, 4, 5, etc.) heteroatoms. In some embodiments, R^{w2} is optionally substituted 5-membered heteroaryl having 1-4 (e.g., 1-3, 1, 2, 3, 4, etc.) heteroatoms. In some embodiments, R^{w2} is optionally substituted 6-membered heteroaryl having 1-4 (e.g., 1-3, 1, 2, 3, 4, etc.) heteroatoms. In some embodiments, R^{w2} is optionally substituted 9-membered bicyclic heteroaryl having 1-4 (e.g., 1-3, 1, 2, 3, 4, etc.) heteroatoms. In some embodiments, R^{w2} is optionally substituted 3-20 (e.g., 3-15, 3-10, 3-9, 3-8, 3-7, 3-6, 3, 4, 5, 6, 7, 8, 9, 10, 11, 12, 13, 14, 15, 16, 17, 18, 19, 20, etc.) membered heterocyclyl having 1-10 (e.g., 1-5, 1-3, 1, 2, 3, 4, 5, 6, 7, 8, 9, 10, etc.) heteroatoms. In some embodiments, R^{w2} is optionally substituted 3-10 (e.g., 4-10, 5-10, 3, 4, 5, 6, 7, 8, 9, 10, etc.) membered heterocyclyl having 1-4 (e.g., 1-3, 1, 2, 3, 4, etc.) heteroatoms. In some embodiments, R^{w2} is optionally substituted 3-10 (e.g., 4-10, 5-10, 3, 4, 5, 6, 7, 8, 9, 10, etc.) membered saturated ring having 0-5 (e.g., 1-5, 1-4, 1-3, 0, 1, 2, 3, 4, 5, etc.) heteroatoms. In some embodiments, R^{w2} is optionally substituted 3-10 (e.g., 4-10, 5-10, 3, 4, 5, 6, 7, 8, 9, 10, etc.) membered partially saturated ring having 0-5 (e.g., 1-5, 1-4, 1-3, 0, 1, 2, 3, 4, 5, etc.) heteroatoms. In some embodiments, R^{w2} is an optionally substituted group which is a combination of two or more of C₁₋₂₀ aliphatic, C₁₋₂₀ heteroaliphatic having 1-10 heteroatoms, C₆₋₂₀ aryl, 5-20 membered heteroaryl having 1-10 heteroatoms, and 3-20 membered heterocyclyl having 1-10 heteroatoms as described herein, wherein the combination has 1-30 (e.g., 1-20, 1-15, 1-10, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, etc.) carbon atoms and 0-10 (e.g., 0-5, 1-10, 1-5, 0, 1, 2, 3, 4, 5, 6, 7, 8, 9, 10, etc.) heteroatoms.

In some embodiments, R^{w2} is or comprises a detectable label, e.g., a fluorescent label, a radioactive label, an label useful in proteomics, an antibody label, a peptide label, etc.

In some embodiments, R^{w2} and R^{w3} are taken together with the nitrogen to which they are attached to form an optionally substituted ring as described herein. In some embodiments, R^{w2} and R^{w3} are taken together with the nitrogen atom to which they are attached to form an optionally substituted 3-14 (e.g., 3-10, 3-8, 4, 5, 6, 7, 8, 9, 10, 11, 12, 13, 14, etc.) membered ring having 0-6 (e.g., 1-6, 1-5, 1-4, 1-3, 0, 1, 2, 3, 4, 5, 6, etc.) heteroatoms in addition to the nitrogen atom. In some embodiments, R^{w1} is —H, and R^{w2} and R^{w3} are each independently optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) aliphatic or are taken together with the nitrogen to which they are attached to form an optionally substituted 3-10 (e.g., 3-7, 4-10, 5-10, 5-7, 3, 4, 5, 6, 7, 8, 9, or 10, etc.) membered ring having 0-3 (e.g., 0, 1-3, 1, 2, 3, etc.) heteroatoms in addition to the nitrogen atom. In some embodiments, R^{w2} and R^{w3} are taken together with the nitrogen to which they are attached to form an optionally substituted 3-10 (e.g., 3-7, 4-10, 5-10, 5-7, 3, 4, 5, 6, 7, 8, 9, or 10, etc.) membered ring having 0-5 (e.g., 1-5, 0, 1-3, 1, 2, 3, 4, 5, etc.) heteroatoms in addition to the nitrogen atom. In some embodiments, R^{w2} and R^{w3} are taken together with the nitrogen to which they are attached to form an optionally substituted 3-10 (e.g., 3-7, 4-10, 5-10, 5-7, 3, 4, 5, 6, 7, 8, 9, 10, etc.) membered ring having 0-3 (e.g., 0, 1-3, 1, 2, 3, etc.) heteroatoms in addition to the nitrogen atom. In some embodiments, R^{w2} and R^{w3} are taken together with the nitrogen to which they are attached to form an optionally substituted 3-8 (e.g., 3-7, 5-7, 3, 4, 5, 6, 7, 8, etc.) membered ring having 0-3 (e.g., 0, 1-3, 1, 2, 3, etc.) heteroatoms in addition to the nitrogen atom. In some embodiments, R^{w2} and R^{w3} are taken together with the nitrogen to which they are attached to form an optionally substituted 5-10 (e.g., 5-9, 5-7, 5, 6, 7, 8, 9, or 10, etc.) membered ring having 0-3 (e.g., 0, 1-3, 1, 2, 3, etc.) heteroatoms in addition to the nitrogen atom. In some embodiments, R^{w2} and R^{w3} are taken together with the nitrogen to which they are attached to form an optionally substituted 5-10 (e.g., 5-9, 5-7, 5, 6, 7, 8, 9, or 10, etc.) membered ring having 0 heteroatom in addition to the nitrogen atom. In some embodiments, R^{w2} and R^{w3} are taken together with the nitrogen to which they are attached to form an optionally substituted 5-10 (e.g., 5-9, 5-7, 5, 6, 7, 8, 9, or 10, etc.) membered ring having 1 heteroatom in addition to the nitrogen atom. In some embodiments, an additional heteroatom is nitrogen. In some embodiments, an additional heteroatom is oxygen. In some embodiments, a formed ring is 3-membered. In some embodiments, it is 4-membered. In some embodiments, it is 5-membered. In some embodiments, it is 6-membered. In some embodiments, it is 7-membered. In some embodiments, it is 8-membered. In some embodiments, it is 9-membered. In some embodiments, it is 10-membered. In some embodiments, a formed ring is saturated. In some embodiments, a formed ring is partially unsaturated. In some embodiments, a formed ring is aromatic. In some embodiments, a formed ring is substituted. In some embodiments, a formed ring is unsubstituted. In some embodiments, there is one heteroatom in the formed ring. In some embodiments, there are two heteroatoms in the formed ring. In some embodiments, there are three heteroatoms in the formed ring. In some embodiments, there are four heteroatoms in the formed ring. In some embodiments, a formed ring is monocyclic. In some embodiments, a formed ring is bicyclic. In some embodiments, a formed ring is polycyclic. In some embodiments, a formed ring is optionally substituted

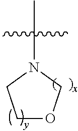

wherein each of x and y is independently 0-4. In some embodiments, a formed ring is optionally substituted

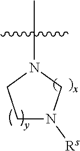

wherein each of x and y is independently 0-4. In some embodiments, a formed ring is optionally substituted

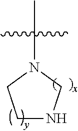

wherein each of x and y is independently 0-4. In some embodiments, a formed ring is optionally substituted

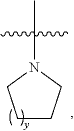

wherein y 0-4. In some embodiments, a formed ring is optionally substituted

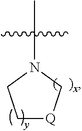

wherein each of x and y is independently 0-4, Q is optionally substituted S, Si, S(O)₂. In some embodiments, Q is S(O)₂. In some embodiments, Q is Si(CH₃)₂.In some embodiments, Q is S. In some embodiments, x is 0. In some embodiments, x is 1. In some embodiments, x is 2. In some embodiments, x is 3. In some embodiments, x is 4. In some embodiments, y is 0. In some embodiments, y is 1. In some embodiments, y is 2. In some embodiments, y is 3. In some embodiments, y is 4.In some embodiments, a formed ring is optionally substituted

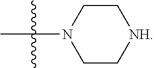 [SMILES: *N1CCNCC1]

In some embodiments, a formed ring is optionally substituted

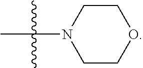 [SMILES: *N1CCOCC1]

In some embodiments, a formed ring is optionally substituted

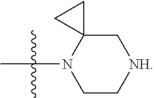 [SMILES: *N1CCNCC12CC2]

In some embodiments, a formed ring is optionally substituted

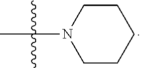 [SMILES: *N1CCCCC1]

In some embodiments, a formed ring is

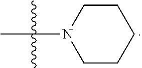 [SMILES: *N1CCCCC1]

In some embodiments, a formed ring is optionally substituted

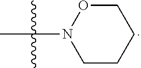 [SMILES: *N1CCCCO1]

In some embodiments, a formed ring is

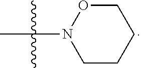 [SMILES: *N1CCCCO1]

In some embodiments, a formed ring is optionally substituted

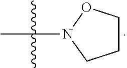 [SMILES: *N1CCCO1]

In some embodiments, a formed ring is

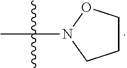 [SMILES: *N1CCCO1]

In some embodiments, a formed ring is optionally substituted

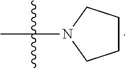 [SMILES: *N1CCCC1]

In some embodiments, a formed ring is optionally substituted

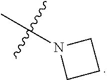 [SMILES: *N1CCC1]

In some embodiments, a formed ring is optionally substituted

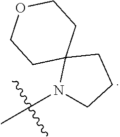 [SMILES: *N1CCCC12CCOCC2]

In some embodiments, —N(R^{w2})(R^{w3}) is optionally substituted —NH₂,

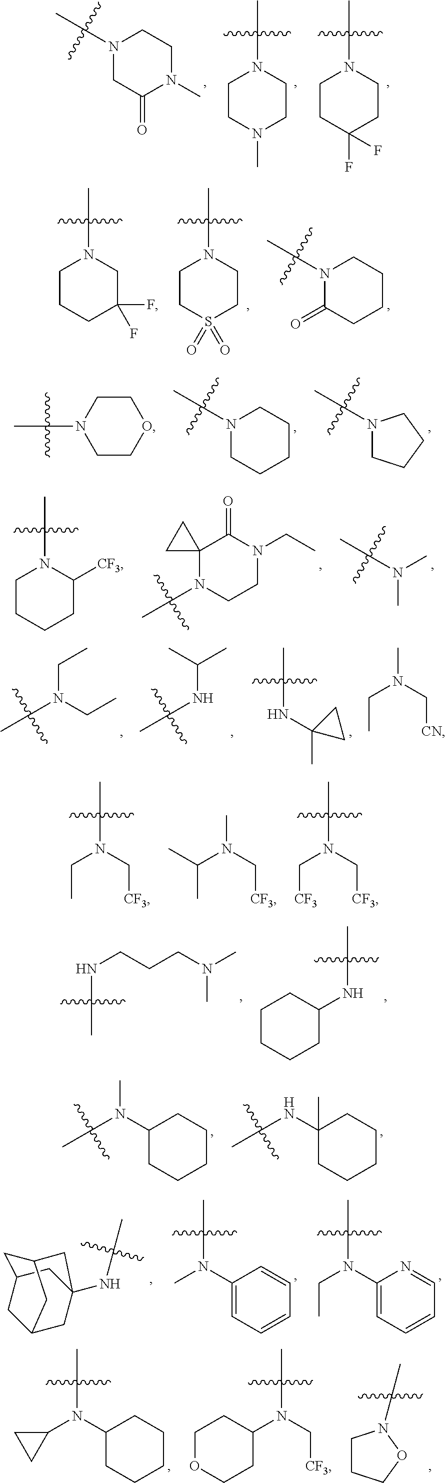

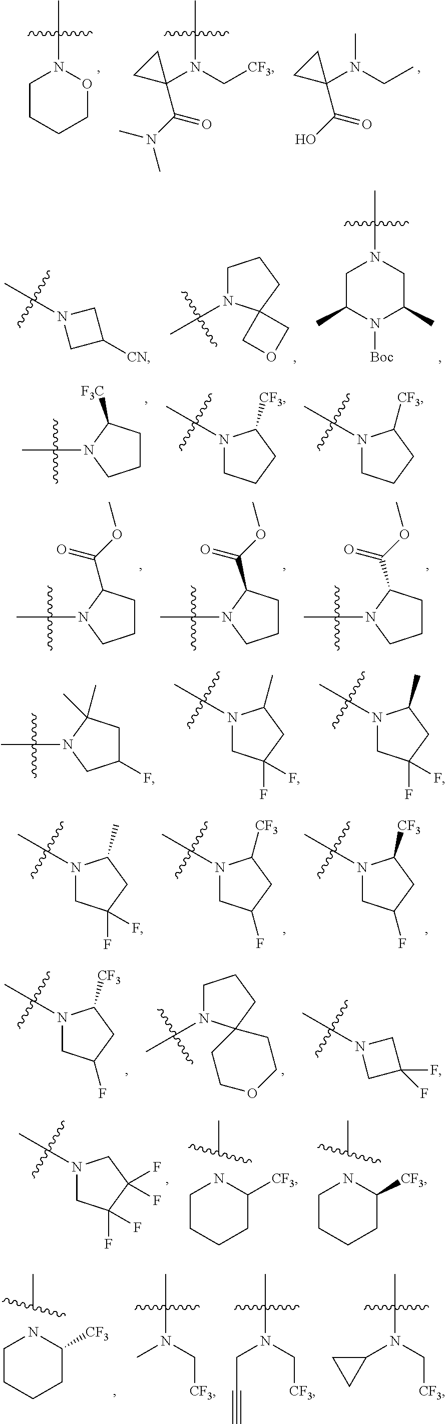

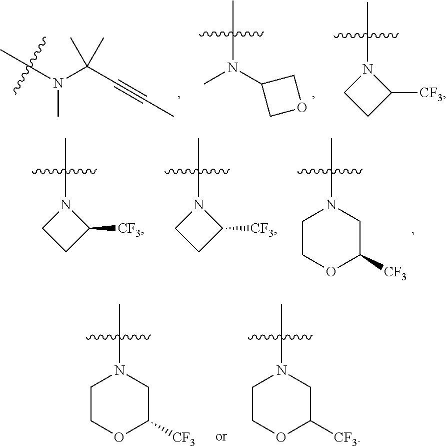

In some embodiments, —N(R^{w2})(R^{w3}) is —NH₂,

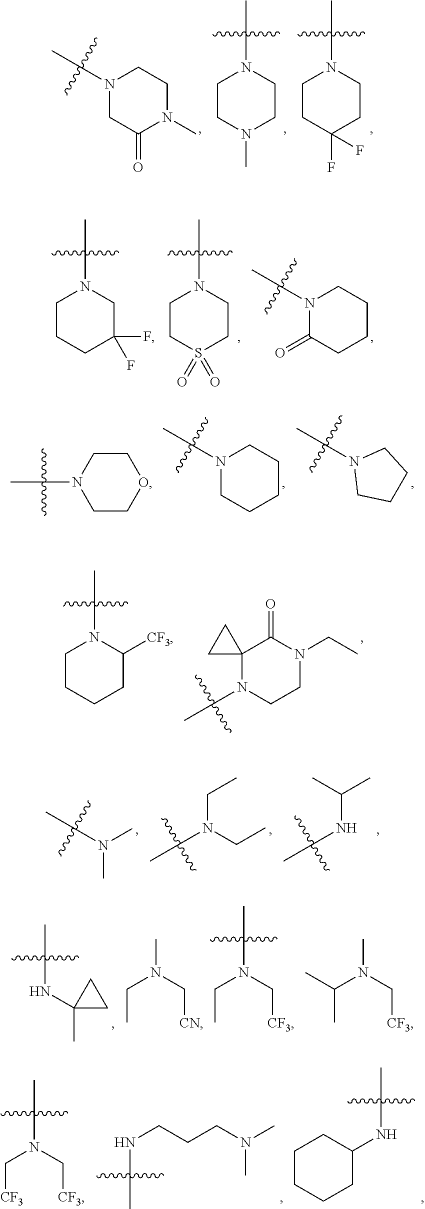

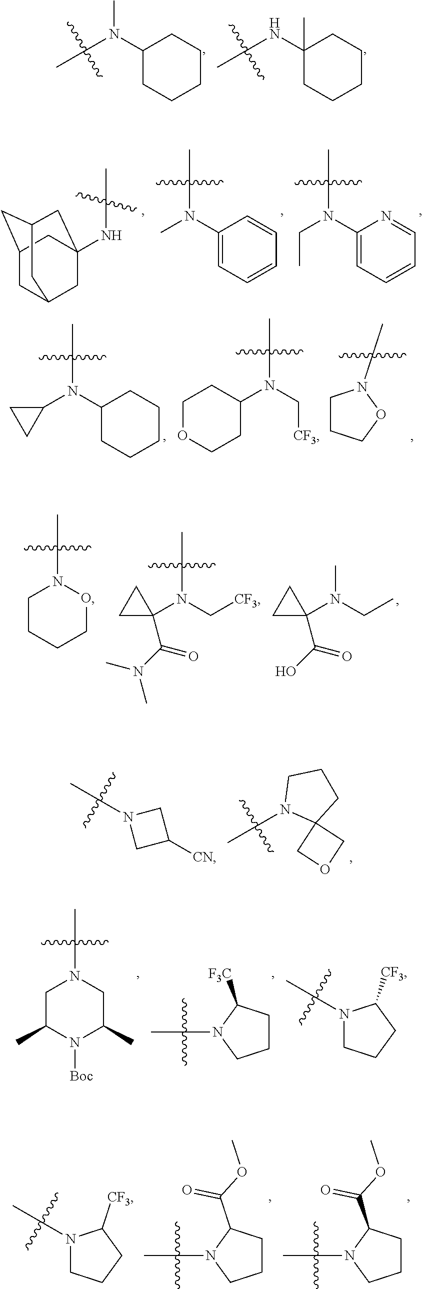

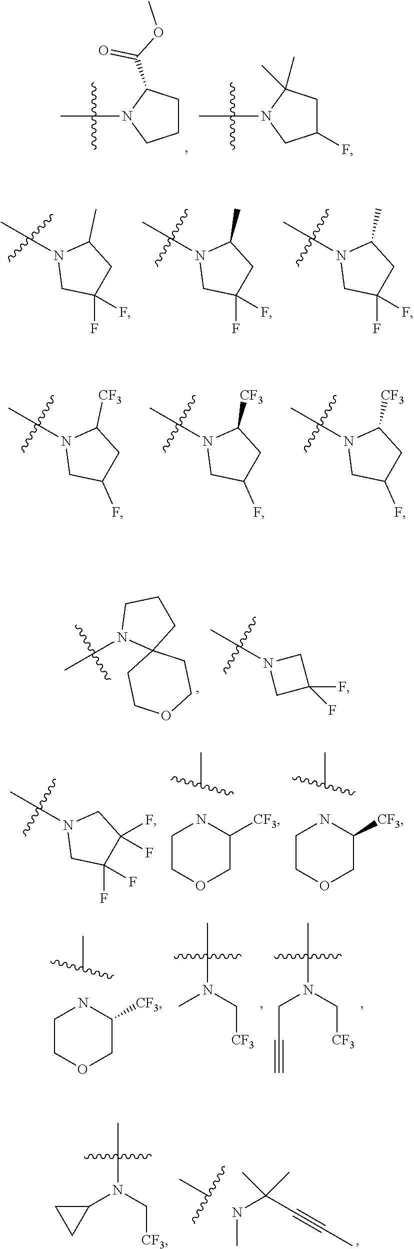

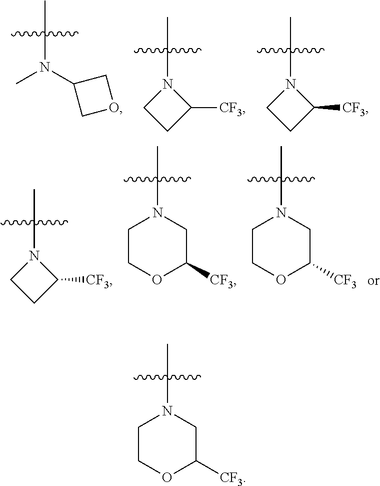

In some embodiments, it is one of such a group. In some embodiments, it is one of such a group which is further substituted as described herein.

### R^{w3}

In some embodiments, R^{w3} is -L-R′ wherein each variable is independently as described herein. For example, in some embodiments, R′ is —H. In some embodiments, R′ is optionally substituted C₁₋₆ aliphatic. In some embodiments, R^{w3} is -L-R′ wherein the methylene unit of L that bonds to R′ is replaced with a moiety, e.g., —N(R′)— as described herein. For example, in some embodiments, R^{w3} is —(CH₂)₃—N(CH₃)—CH₃. In some embodiments, R^{w3} is R′ as described herein. In some embodiments, R^{w3} is R as described herein.

In some embodiments, R^{w3} is R as described herein. For example, in some embodiments, R^{w3} is —H. In some embodiments, R^{w3} is optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) aliphatic. In some embodiments, R^{w3} is optionally substituted C₁₋₁₀ alkyl. In some embodiments, R^{w3} is optionally substituted methyl. In some embodiments, R^{w3} is methyl. In some embodiments, R^{w3} is optionally substituted ethyl. In some embodiments, R^{w3} is ethyl. In some embodiments, R^{w3} is —CH₂CF₃. In some embodiments, R^{w3} is optionally substituted propyl. In some embodiments, R^{w3} is optionally substituted isopropyl. In some embodiments, R^{w3} is propyl. In some embodiments, R^{w3} is isopropyl. In some embodiments, R^{w3} is optionally substituted C₃₋₁₀ cycloaliphatic. In some embodiments, R^{w3} is optionally substituted C₃₋₁₀ cycloalkyl. In some embodiments, R^{w3} is optionally substituted cyclopropyl. In some embodiments, R^{w} is cyclobutyl. In some embodiments, R^{w3} is cyclopentyl. In some embodiments, R^{w3} is cyclohexyl. In some embodiments, R^{w3} is optionally substituted adamantyl. In some embodiments, R^{w3} is optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) heteroaliphatic having 1-6 (e.g., 1-3, 1-2, 1, 2, 3, 4, 5, 6, etc.) heteroatoms. In some embodiments, R^{w3} is optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) heteroaliphatic having 1, 2 or 3 heteroatoms. In some embodiments, R^{w3} is optionally substituted aryl. In some embodiments, R^{w3} is optionally substituted 6-14 membered (e.g., 6, 10, 14, etc.) aryl. In some embodiments, R^{w3} is optionally substituted phenyl. In some embodiments, R^{w3} is optionally substituted 5-20 (e.g., 5-14, 5-10, 5-9, 5, 6, 8, 9, etc.) membered heteroaryl having 1-10 (e.g., 1-5, 1-4, 1-3, 1, 2, 3, 4, 5, 6, 7, 8, 9, 10, etc.) heteroatoms. In some embodiments, R^{w3} is optionally substituted 5-10 membered heteroaryl having 1-5 (e.g., 1-4, 1-3, 1, 2, 3, 4, 5, etc.) heteroatoms. In some embodiments, R^{w3} is optionally substituted 5-membered heteroaryl having 1-4 (e.g., 1-3, 1, 2, 3, 4, etc.) heteroatoms. In some embodiments, R^{w3} is optionally substituted 6-membered heteroaryl having 1-4 (e.g., 1-3, 1, 2, 3, 4, etc.) heteroatoms. In some embodiments, R^{w3} is 2-pyridyl. In some embodiments, R^{w3} is 3-pyridyl. In some embodiments, R^{w3} is 4-pyridyl. In some embodiments, R^{w3} is optionally substituted 9-membered bicyclic heteroaryl having 1-4 (e.g., 1-3, 1, 2, 3, 4, etc.) heteroatoms. In some embodiments, R^{w3} is optionally substituted 3-20 (e.g., 3-15, 3-10, 3-9, 3-8, 3-7, 3-6, 3, 4, 5, 6, 7, 8, 9, 10, 11, 12, 13, 14, 15, 16, 17, 18, 19, 20, etc.) membered heterocyclyl having 1-10 (e.g., 1-5, 1-3, 1, 2, 3, 4, 5, 6, 7, 8, 9, 10, etc.) heteroatoms. In some embodiments, R″ is optionally substituted 3-10 (e.g., 4-10, 5-10, 3, 4, 5, 6, 7, 8, 9, 10, etc.) membered heterocyclyl having 1-4 (e.g., 1-3, 1, 2, 3, 4, etc.) heteroatoms. In some embodiments, R^{w3} is optionally substituted 3-10 (e.g., 4-10, 5-10, 3, 4, 5, 6, 7, 8, 9, 10, etc.) membered saturated ring having 0-5 (e.g., 1-5, 1-4, 1-3, 0, 1, 2, 3, 4, 5, etc.) heteroatoms. In some embodiments, R^{w3} is optionally substituted 3-10 (e.g., 4-10, 5-10, 3, 4, 5, 6, 7, 8, 9, 10, etc.) membered partially saturated ring having 0-5 (e.g., 1-5, 1-4, 1-3, 0, 1, 2, 3, 4, 5, etc.) heteroatoms. In some embodiments, R^{w3} is an optionally substituted group which is a combination of two or more of C₁₋₂₀ aliphatic, C₁₋₂₀ heteroaliphatic having 1-10 heteroatoms, C₆₋₂₀ aryl, 5-20 membered heteroaryl having 1-10 heteroatoms, and 3-20 membered heterocyclyl having 1-10 heteroatoms as described herein, wherein the combination has 1-30 (e.g., 1-20, 1-15, 1-10, 1,2,3,4,5,6,7,8,9, 10, 11, 12, 13, 14, 15, 16, 17, 18, 19, 20, 21, 22, 23, 24, 25, 26, 27, 28, 29, 30, etc.) carbon atoms and 0-10 (e.g., 0-5, 1-10, 1-5, 0, 1, 2, 3, 4, 5, 6, 7, 8, 9, 10, etc.) heteroatoms.

In some embodiments, R^{w3} is or comprises a detectable label, e.g., a fluorescent label, a radioactive label, an label useful in proteomics, an antibody label, a peptide label, etc.

In some embodiments, both R^{w1} and R^{w3} are R as described herein. In some embodiments, R^{w1} and R^{w3} are taken together with their intervening atoms to form an optionally substituted ring as described herein. In some embodiments, both R^{w2} and R^{w3} are R as described herein. In some embodiments, R^{w2} and R^{w3} are taken together with the nitrogen to which they are attached to form an optionally substituted ring as described herein. In some embodiments, each of R^{w1}, R^{w2} and R^{w3} is independently R as described herein. In some embodiments, R^{w1}, R^{w2} and R^{w3} are taken together with their intervening atoms to form an optionally substituted ring as described herein.

### R^{wh}

In some embodiments, R^{wh} is —OH. In some embodiments, R^{wh} is —SH.

### X

As described herein, X is —O—, —S—, —C(R¹)═, —N(R¹)—, or optionally substituted —CH═, —CH═CH—, or —NH—. In some embodiments, X is —O—. In some embodiments, X is —S—. In some embodiments, X is —C(R′)═ wherein R¹ is as described herein. In some embodiments, X is —N(R¹)— wherein R¹ is as described herein. In some embodiments, X is optionally substituted —CH═. In some embodiments, X is —CH═. In some embodiments, X is optionally substituted —CH═CH—. In some embodiments, X is —CH═CH—. In some embodiments, X is optionally substituted —NH—. In some embodiments, X is —NH—.

### Y

As described herein, Y is C or N. In some embodiments, Y is C. In some embodiments, Y in N.

### Z

As described herein, Z is C or N. In some embodiments, Z is C. In some embodiments, Z in N.

[symbol] , e.g., the bond between Y and Z, can be a single or double bond. In some embodiments, it is a single bond. In some embodiments, it is a double bond. For example, in some embodiments, Y and Z are C and the bond is a double bond.

### R¹

As described herein, R′ is R⁹ as described herein. In some embodiments, R¹ is R″, halogen, —CN, oxo, —NO₂, or an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl. In some embodiments, R¹ is R′ as described herein. In some embodiments, R¹ is R as described herein.

In some embodiments, R¹ is hydrogen. In some embodiments, R¹ is not hydrogen. In some embodiments, R¹ is R′ as described herein. In some embodiments, R′ is R as described herein.

### R²

As described herein, R² is R″ or

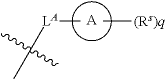

wherein each variable is independently as described herein.

In some embodiments, R² is R″ as described herein. In some embodiments, R² is hydrogen. In some embodiments, R² is not hydrogen. In some embodiments, R² is R′ as described herein. In some embodiments, R² is R as described herein. For example, in some embodiments, R is optionally substituted phenyl. In some embodiments, R is 4-flurophenyl. In some embodiments, R is 4-aminophenyl. In some embodiments, R is optionally substituted 5-6 membered heteroaryl having 1-3 heteroatoms independently selected from nitrogen, oxygen and sulfur.

In some embodiments, R² is -L-R′ wherein each of L and R′ is independently as described herein. For example, in some embodiments, L is a covalent bond. In some embodiments, L is optionally substituted —CH₂—. In some embodiments, L is -Cy- as described herein. In some embodiments, L is optionally substituted phenylene. In some embodiments, L is optionally substituted 1,4-phenylene. In some embodiments, R′ is R as described herein. In some embodiments, R′ is optionally substituted C₁₋₆ (e.g., C₁₋₅, C₁, C₂, C₃, C₄, C₅, C₆, etc.) aliphatic. In some embodiments, R′ is an optionally substituted 3-10 (e.g., 4-10, 5-10, 3, 4, 5, 6, 7, 8, 9, 10, etc.) membered saturated ring having 0-4 (e.g., 1-4, 1-3, 0, 1, 2, 3, 4, etc.) heteroatoms. In some embodiments, R′ is optionally substituted

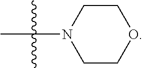 [SMILES: *N1CCOCC1]

In some embodiments, R² is

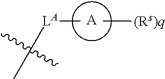

wherein each variable is independently as described herein.

In some embodiments, L^{A} is a covalent bond. In some embodiments, a compound of formula A has the structure of formula A-I. In some embodiments, a compound of formula A has the structure of formula A-II. In some embodiments, a compound of formula A has the structure of formula A-III.

### R³

As described herein, R³ is R″ or

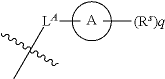

wherein each variable is independently as described herein.

In some embodiments, R³ is R″ as described herein. In some embodiments, R³ is hydrogen. In some embodiments, R³ is not hydrogen. In some embodiments, R³ is R′ as described herein. In some embodiments, R³ is R as described herein. For example, in some embodiments, R is optionally substituted phenyl. In some embodiments, R is 4-flurophenyl. In some embodiments, R is 4-aminophenyl. In some embodiments, R is optionally substituted 5-6 membered heteroaryl having 1-3 heteroatoms independently selected from nitrogen, oxygen and sulfur.

In some embodiments, R³ is

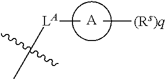

wherein each variable is independently as described herein.

In some embodiments, one of R² and R³ is —H. In some embodiments, one of R² and R³ is —H and the other is not —H. In some embodiments, R² is —H and R³ is not —H. In some embodiments, R² is not —H and R³ is H. In some embodiments, the atom to which R² and R³ is attached is chiral. In some embodiments, it is R. In some embodiments, it is S. In some embodiments, stereopurity with respect to the carbon atom to which R² and R³ is attached is about or at least about 60%, 65%, 70%, 75%, 80%, 85%, 90%, 95%, 96%, 97%, 98%, or 99%. In some embodiments, the two R/S configurations exist at about the same level of about 50%.

### R⁴

In some embodiments, R⁴ comprises an isourea moiety. In some embodiments, R⁴ is an isourea moiety. In some embodiments, R⁴ comprises an isothiourea moiety. In some embodiments, R⁴ is an isothiourea moiety. In some embodiments, R⁴ is -L^{w}-R^{w}, wherein each variable is independently as described herein. In some embodiments, R⁴ is R^{w} as described herein. In some embodiments, R⁴ is —O—C(═NR^{w1})N(R^{w2})(R^{w3}) wherein each variable is independently as described herein. In some embodiments, R⁴ is —S—C(═NR^{w1})N(R^{w2})(R^{w3}) wherein each variable is independently as described herein.

In some embodiments, R⁴ is

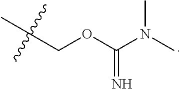 [SMILES: *COC(=N)N(C)C]

In some embodiments, R′ is

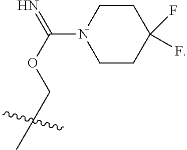 [SMILES: *COC(=N)N1CCC(F)(F)CC1]

In some embodiments, R⁴ is

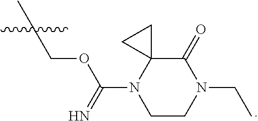 [SMILES: *COC(=N)N1CCN(CC)C(=O)C12CC2]

In some embodiments, R⁴ is

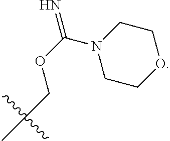 [SMILES: *COC(=N)N1CCOCC1]

In some embodiments, R⁴ is

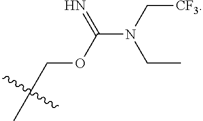 [SMILES: *COC(=N)N(CC)CC(F)(F)F]

In some embodiments, R⁴ is

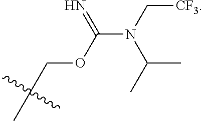 [SMILES: *COC(=N)N(CC(F)(F)F)C(C)C]

In some embodiments, R⁴ is

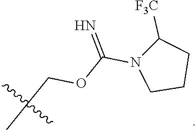 [SMILES: *COC(=N)N1CCCC1C(F)(F)F]

In some embodiments, R⁴ is

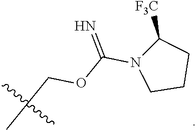 [SMILES: *COC(=N)N1CCC[C@@H]1C(F)(F)F]

In some embodiments, R⁴ is

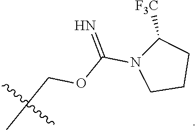 [SMILES: *COC(=N)N1CCC[C@H]1C(F)(F)F]

In some embodiments, R⁴ is

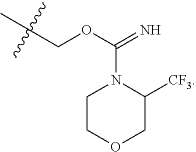 [SMILES: *COC(=N)N1CCOCC1C(F)(F)F]

In some embodiments, R⁴ is

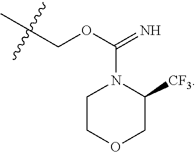 [SMILES: *COC(=N)N1CCOC[C@@H]1C(F)(F)F]

In some embodiments, R⁴ is

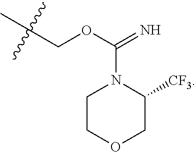 [SMILES: *COC(=N)N1CCOC[C@H]1C(F)(F)F]

In some embodiments, R⁴ is

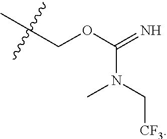 [SMILES: *COC(=N)N(C)CC(F)(F)F]

In some embodiments, R⁴ is

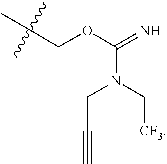 [SMILES: *COC(=N)N(CC#C)CC(F)(F)F]

In some embodiments, R⁴ is

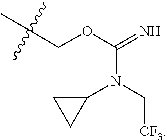 [SMILES: *COC(=N)N(CC(F)(F)F)C1CC1]

In some embodiments, R⁴ is

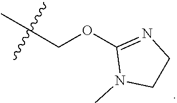 [SMILES: *COC1=NCCN1C]

In some embodiments, R⁴ is

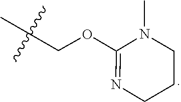 [SMILES: *COC1=NCCCN1C]

In some embodiments, R⁴ is

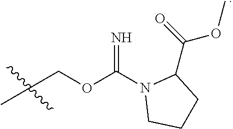 [SMILES: *COC(=N)N1CCCC1C(=O)OC ; "."]

In some embodiments, R⁴ is

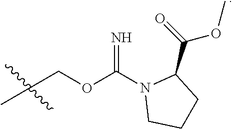 [SMILES: *COC(=N)N1CCC[C@@H]1C(=O)OC ; "."]

In some embodiments, R⁴ is

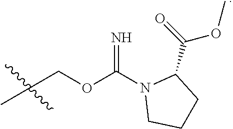 [SMILES: *COC(=N)N1CCC[C@H]1C(=O)OC ; "."]

In some embodiments, R⁴ is

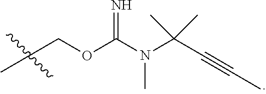 [SMILES: *COC(=N)N(C)C(C)(C)C#CC]

In some embodiments, R⁴ is

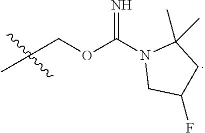 [SMILES: *COC(=N)N1CC(F)CC1(C)C]

In some embodiments, R⁴ is

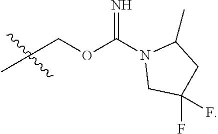 [SMILES: *COC(=N)N1CC(F)(F)CC1C]

In some embodiments, R⁴ is

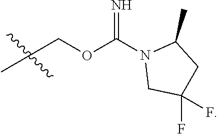 [SMILES: *COC(=N)N1CC(F)(F)C[C@@H]1C]

In some embodiments, R⁴ is

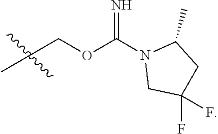 [SMILES: *COC(=N)N1CC(F)(F)C[C@H]1C]

In some embodiments, R⁴ is

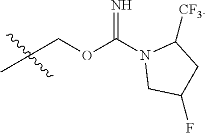 [SMILES: *COC(=N)N1CC(F)CC1C(F)(F)F]

In some embodiments, R⁴ is

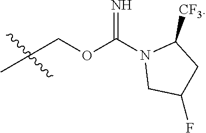 [SMILES: *COC(=N)N1CC(F)C[C@@H]1C(F)(F)F]

In embodiments, R⁴ is

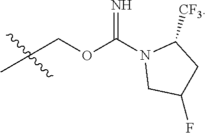 [SMILES: *COC(=N)N1CC(F)C[C@H]1C(F)(F)F]

In some embodiments, R⁴ is

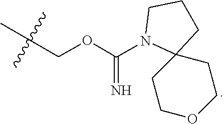 [SMILES: *COC(=N)N1CCCC12CCOCC2]

In some embodiments, R⁴ is

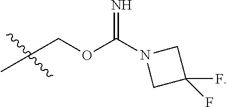 [SMILES: *COC(=N)N1CC(F)(F)C1]

In some embodiments, R⁴ is

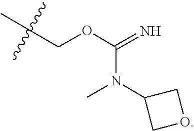 [SMILES: *COC(=N)N(C)C1COC1]

In some embodiments, R⁴ is

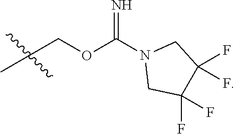 [SMILES: *COC(=N)N1CC(F)(F)C(F)(F)C1]

In some embodiments, R⁴ is

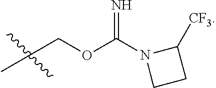 [SMILES: *COC(=N)N1CCC1C(F)(F)F]

In some embodiments, R⁴ is

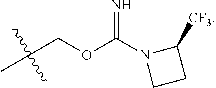 [SMILES: *COC(=N)N1CC[C@@H]1C(F)(F)F]

In some embodiments, R⁴ is

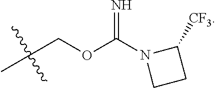 [SMILES: *COC(=N)N1CC[C@H]1C(F)(F)F]

In some embodiments, R⁴ is

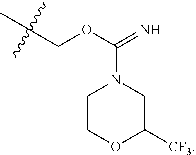 [SMILES: *COC(=N)N1CCOC(C(F)(F)F)C1]

In some embodiments, R⁴ is

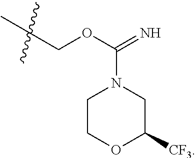 [SMILES: *COC(=N)N1CCO[C@H](C(F)(F)F)C1]

In some embodiments, R⁴ is

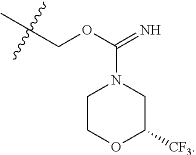 [SMILES: *COC(=N)N1CCO[C@@H](C(F)(F)F)C1]

In some embodiments, R⁴ is

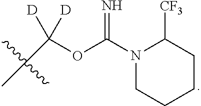 [SMILES: *C([2H])([2H])OC(=N)N1CCCCC1C(F)(F)F]

In some embodiments, R⁴ is

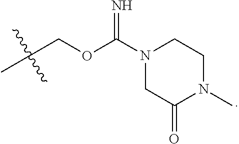 [SMILES: *COC(=N)N1CCN(C)C(=O)C1]

In some embodiments, R⁴ is

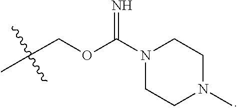 [SMILES: *COC(=N)N1CCN(C)CC1]

In some embodiments, R⁴ is

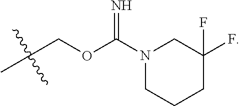 [SMILES: *COC(=N)N1CCCC(F)(F)C1]

In some embodiments, R⁴ is

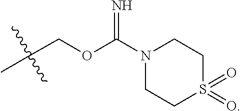 [SMILES: *COC(=N)N1CCS(=O)(=O)CC1]

In some embodiments, R⁴ is

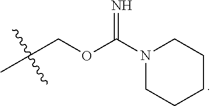 [SMILES: *COC(=N)N1CCCCC1]

In some embodiments, R⁴ is

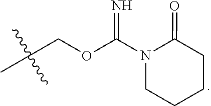 [SMILES: *COC(=N)N1CCCCC1=O]

In some embodiments, R⁴ is

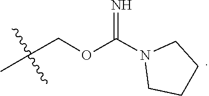 [SMILES: *COC(=N)N1CCCC1]

In some embodiments, R⁴ is

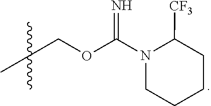 [SMILES: *COC(=N)N1CCCCC1C(F)(F)F]

In some embodiments, R⁴ is

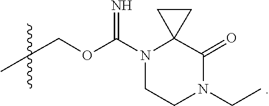 [SMILES: *COC(=N)N1CCN(CC)C(=O)C12CC2]

In some embodiments, R⁴ is

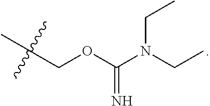 [SMILES: *COC(=N)N(CC)CC]

In some embodiments, R⁴ is

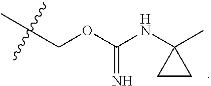 [SMILES: *COC(=N)NC1(C)CC1]

In some embodiments, R⁴ is

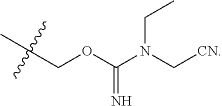 [SMILES: *COC(=N)N(CC)CC#N]

In some embodiments, R⁴ is

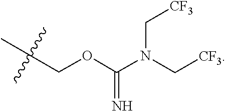 [SMILES: *COC(=N)N(CC(F)(F)F)CC(F)(F)F]

In some embodiments, R⁴ is

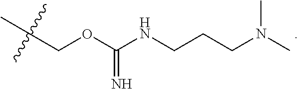 [SMILES: *COC(=N)NCCCN(C)C]

In some embodiments, R⁴ is

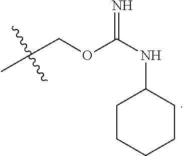 [SMILES: *COC(=N)NC1CCCCC1]

In some embodiments, R⁴ is

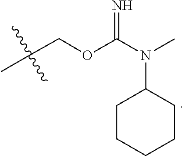 [SMILES: *COC(=N)N(C)C1CCCCC1]

In some embodiments, R⁴ is

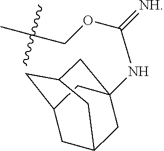 [SMILES: *COC(=N)NC12CC3CC(CC(C3)C1)C2]

In some embodiments, R⁴ is

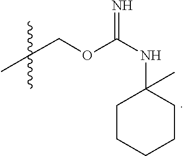 [SMILES: *COC(=N)NC1(C)CCCCC1]

In some embodiments, R⁴ is

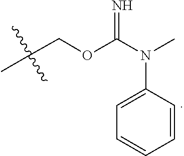 [SMILES: *COC(=N)N(C)c1ccccc1]

In some embodiments, R⁴ is

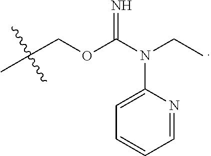 [SMILES: *COC(=N)N(CC)c1ccccn1]

In some embodiments, R⁴ is

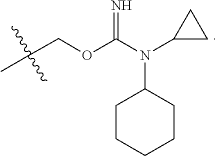 [SMILES: *COC(=N)N(C1CCCCC1)C1CC1]

In some embodiments, R⁴ is

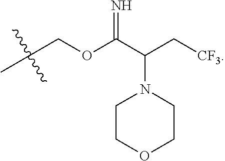 [SMILES: *COC(=N)C(CC(F)(F)F)N1CCOCC1]

In some some embodiments, R⁴ is

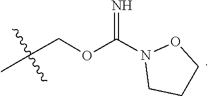 [SMILES: *COC(=N)N1CCCO1]

In some embodiments, R⁴ is

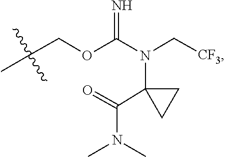 [SMILES: *COC(=N)N(CC(F)(F)F)C1(C(=O)N(C)C)CC1]

In some embodiments, R⁴ is

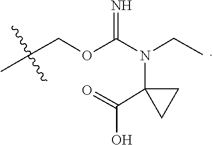 [SMILES: *COC(=N)N(CC)C1(C(=O)O)CC1]

In some embodiments, R⁴ is

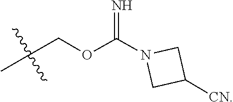 [SMILES: *COC(=N)N1CC(C#N)C1]

In some some embodiments, R⁴ is

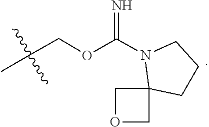 [SMILES: *COC(=N)N1CCCC12COC2]

In some embodiments, R⁴ is

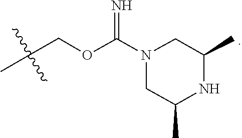 [SMILES: *COC(=N)N1C[C@@H](C)N[C@@H](C)C1]

In some embodiments, R⁴ is

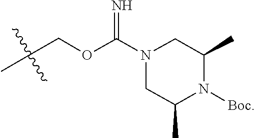 [SMILES: *COC(=N)N1C[C@@H](C)N(C(=O)OC(C)(C)C)[C@@H](C)C1]

In some embodiments, R⁴ is

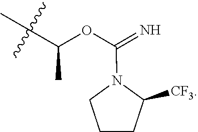 [SMILES: *[C@H](C)OC(=N)N1CCC[C@@H]1C(F)(F)F]

In some embodiments, R⁴ is

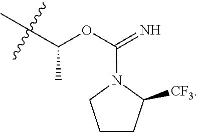 [SMILES: *[C@@H](C)OC(=N)N1CCC[C@@H]1C(F)(F)F]

In some embodiments, R⁴ is

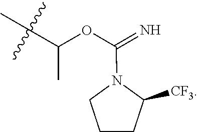 [SMILES: *C(C)OC(=N)N1CCC[C@@H]1C(F)(F)F]

In some embodiments, R⁴ is

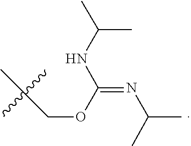 [SMILES: *CO/C(=N\C(C)C)NC(C)C]

In some embodiments, R⁴ is

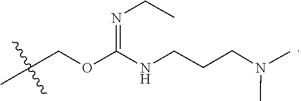 [SMILES: *CO/C(=N/CC)NCCCN(C)C]

In some embodiments, R⁴ is

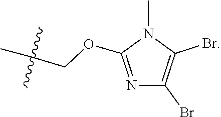 [SMILES: *COc1nc(Br)c(Br)n1C]

In some embodiments, R⁴ is

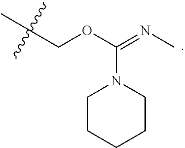 [SMILES: *CO/C(=N/C)N1CCCCC1]

In some embodiments, R⁴ is

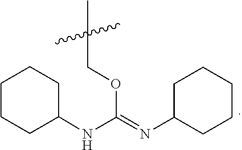 [SMILES: *CO/C(=N\C1CCCCC1)NC1CCCCC1]

In some embodiments, R⁴ is

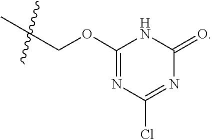 [SMILES: *COc1nc(Cl)nc(=O)[nH]1]

In some embodiments, R⁴ is

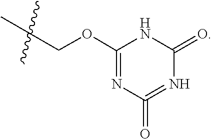

In some embodiments, R⁴ is

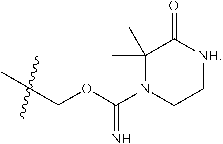 [SMILES: *COC(=N)N1CCNC(=O)C1(C)C]

In some embodiments, R⁴ is

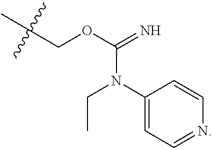 [SMILES: *COC(=N)N(CC)c1ccncc1]

In some embodiments, R⁴ is -L^{w}-R^{wh}, wherein each variable is independently as described herein.

### R⁵

In some embodiments, R⁵ is R″ as described herein. In some embodiments, R⁵ is —C(O)OR″ wherein R″ is as described herein. In some embodiments, R⁵ is —C(O)N(R′)₂ wherein each R′ is independently as described herein. In some embodiments, R⁵ is —C(O)NHR′ wherein R′ is as described herein. In some embodiments, R⁵ is —C(O)NH₂. In some embodiments, R⁵ is —C(O)OR′ wherein R′ is as described herein. For example, in some embodiments, R′ is optionally substituted C₁₋₆ aliphatic. In some embodiments, R⁵ is —C(O)OCH₃. In some embodiments, R⁵ is

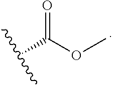

In some embodiments, R⁵ is

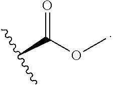

In some embodiments, R⁵ is R as described herein. For example, in some embodiments, R⁵ is —H. In some embodiments, R⁵ is optionally substituted C₁₋₁₀ (e.g., C₁₋₉, C₁₋₈, C₁₋₆, C₂₋₁₀, C₃₋₁₀, C₄₋₁₀, C₁, C₂, C₃, C₄, C₅, C₆, C₇, C₈, C₉, C₁₀, etc.) aliphatic. In some embodiments, R⁵ is optionally substituted C₁₋₆ aliphatic. In some embodiments, R⁵ is —CH₃. In some embodiments, R⁵ is optionally substituted C₂₋₁₀ aliphatic. In some embodiments, R⁵ is optionally substituted C₃₋₁₀ aliphatic. In some embodiments, R⁵ is optionally substituted C₄₋₁₀ aliphatic. In some embodiments, R⁵ is optionally substituted C₁₋₁₀ (e.g., C₁₋₉, C₁₋₈, C₁₋₆, C₂₋₁₀, C₃₋₁₀, C₄₋₁₀, C₁, C₂, C₃, C₄, C₅, C₆, C₇, C₈, C₉, C₁₀, etc.) alkyl. In some embodiments, R⁵ is optionally substituted C₁₋₆ alkyl. In some embodiments, R⁵ is optionally substituted C₂₋₁₀ alkyl. In some embodiments, R^{s} is optionally substituted C₃₋₁₀ alkyl. In some embodiments, R⁵ is optionally substituted C₄₋₁₀ alkyl. In some embodiments, R⁵ is C₁₋₆ alkyl or haloC₁₋₆ alkyl. In some embodiments, R⁵ is C₁₋₆ alkyl optionally substituted with one or more —F. In some embodiments, R⁵ is methyl. In some embodiments, R⁵ is optionally substituted n-butyl. In some embodiments, R⁵ is n-butyl.

### R⁶

In some embodiments, R⁶ is R″ as described herein. In some embodiments, R⁶ is —C(O)OR″ wherein R″ is as described herein. In some embodiments, R⁶ is —C(O)N(R′)₂ wherein each R′ is independently as described herein. In some embodiments, R⁶ is —C(O)NHR′ wherein R′ is as described herein. In some embodiments, R⁶ is —C(O)NH₂. In some embodiments, R⁶ is —C(O)OR′ wherein R′ is as described herein. For example, in some embodiments, R′ is optionally substituted C₁₋₆ aliphatic. In some embodiments, R⁶ is —C(O)OCH₃. In some embodiments, R⁶ is

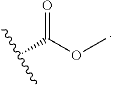

In some embodiments, R⁶ is

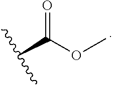

In some embodiments, R⁶ is R as described herein. For example, in some embodiments, R⁶ is optionally substituted C₁₋₁₀ (e.g., C₁₋₉, C₁₋₈, C₁₋₆, C₂₋₁₀, C₃₋₁₀, C₄₋₁₀, C₁, C₂, C₃, C₄, C₅, C₆, C₇, C₈, C₉, C₁₀, etc.) aliphatic. In some embodiments, R⁶ is optionally substituted C₂₋₁₀ aliphatic. In some embodiments, R⁶ is optionally substituted C₃₋₁₀ aliphatic. In some embodiments, R⁶ is optionally substituted C₄₋₁₀ aliphatic. In some embodiments, R⁶ is optionally substituted C₁₋₁₀ (e.g., C₁₋₉, C₁₋₈, C₁₋₆, C₂₋₁₀, C₃₋₁₀, C₄₋₁₀, C₁, C₂, C₃, C₄, C₅, C₆, C₇, C₈, C₉, C₁₀, etc.) alkyl. In some embodiments, R⁶ is optionally substituted C₂₋₁₀ alkyl. In some embodiments, R⁶ is optionally substituted C₃₋₁₀ alkyl. In some embodiments, R⁶ is optionally substituted C₄₋₁₀ alkyl. In some embodiments, R⁶ is optionally substituted n-butyl. In some embodiments, R⁶ is n-butyl.

In some embodiments, at least one of R⁵ and R⁶ is —H. In some embodiments, one of R^{s} is —H and the other is not —H. In some embodiments, the carbon atom to which R⁵ and R⁶ is attached is chiral. In some embodiments, it is R. In some embodiments, it is S. In some embodiments, stereopurity with respect to the carbon atom to which R⁵ and R⁶ is attached is about or at least about 60%, 65%, 70%, 75%, 80%, 85%, 90%, 95%, 96%, 97%, 98%, or 99%. In some embodiments, the two R/S configurations exist at about the same level of about 50%.

In some embodiments, one of R² and R³ is —H, and one of R⁵ and R⁶ is —H. Alternatively or additionally, in some embodiments, one of R² and R³ is not-H, and one of R⁵ and R⁶ is not —H. In some embodiments, one of R² and R³ is not-H, one of R⁵ and R⁶ is not —H, and they are trans. In some embodiments, one of R² and R³ is not-H, one of R⁵ and R⁶ is not —H, and they are cis. In some embodiments, one of R² and R³ is —H, one of R and R′ is —H, and they are trans. In some embodiments, one of R² and R³ is —H, one of R⁵ and R⁶ is —H, and they are cis. In some embodiments, the carbon to which R′ is attached has the configuration of

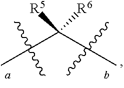

wherein the carbon is bonded to the carbon to which R⁷ is bonded through bond a, and to the nitrogen atom through bond b. In some embodiments, the carbon to which R⁵ is attached has the configuration of

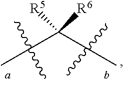

wherein the carbon is bonded to the carbon to which R⁷ is bonded through bond a, and to the nitrogen atom through bond b.

### R⁷

In some embodiments, R⁷ is R″ as described herein. In some embodiments, R⁷ is R′ as described herein. In some embodiments, R⁷ is R as described herein. In some embodiments, R⁷ is —H.

### R⁸

In some embodiments, R′ is R″ as described herein. In some embodiments, R′ is R′ as described herein. In some embodiments, R′ is R as described herein. In some embodiments, R′ is —H.

### R⁹

In some embodiments, R⁹ is R″ as described herein. In some embodiments, R⁹ is R′ as described herein. In some embodiments, R⁹ is R as described herein.

In some embodiments, R⁹ is —OR wherein R is as described herein. In some embodiments, R⁹ is —OR wherein R is optionally substituted C₁₋₆ aliphatic. In some embodiments, R⁹ is —OCH₃.

In some embodiments, R⁹ is —N(R′)₂ wherein each R′ is independently as described herein. In some embodiments, R⁹ is —N(R′)₂ wherein one R′ is —H and the other is optionally substituted C₁₀ aliphatic. In some embodiments, the other is optionally substituted adamantyl. In some embodiments, the other is 1-adamentyl.

In some embodiments, R⁹ is —H. In some embodiments, R⁹ is not —H.

In some embodiments, R⁹ is halogen. In some embodiments, R⁹ is —F. In some embodiments, R⁹ is —Cl. In some embodiments, R⁹ is —Br. In some embodiments, R⁹ is —I. In some embodiments, R⁹ is —CN. In some embodiments, R⁹ is oxo. In some embodiments, R⁹ is —NO₂. In some embodiments, R⁹ is optionally substituted acyl. In some embodiments, R⁹ is optionally substituted acylamino. In some embodiments, R⁹ is hydroxy. In some embodiments, R⁹ is optionally substituted amino acid. In some embodiments, R⁹ is optionally substituted amine. In some embodiments, R⁹ is optionally substituted amide. In some embodiments, R⁹ is optionally substituted carbamate. In some embodiments, R⁹ is optionally substituted ester. In some embodiments, R⁹ is optionally substituted ether. In some embodiments, R⁹ is optionally substituted carboxylic acid. In some embodiments, R⁹ is optionally substituted thio. In some embodiments, R⁹ is optionally substituted thioalkyl. In some embodiments, R⁹ is optionally substituted thioester. In some embodiments, R⁹ is optionally substituted thioether. In some embodiments, R⁹ is optionally substituted sulfate. In some embodiments, R⁹ is optionally substituted sulfonamide. In some embodiments, R⁹ is optionally substituted sulfoxide. In some embodiments, R⁹ is optionally substituted sulfonate. In some embodiments, R⁹ is optionally substituted sulfone. In some embodiments, R⁹ is optionally substituted alkylsulfonyl. In some embodiments, R⁹ is optionally substituted arylsulfonyl. In some embodiments, when there are two or more R⁹, they can be the same or different and each is independently as described herein.

In some embodiments, there is one occurrence of R⁹ bonded to a moiety, e.g., a ring. In some embodiments, two or more occurrences of R⁹ are bonded to a moiety, e.g., a ring, wherein each occurrence of R⁹ is independently as described herein. In some embodiments, an occurrence of R⁹ is halogen. In some embodiments, an occurrence of R⁹ is —F. In some embodiments, an occurrence of R⁹ is —N(R′)₂. In some embodiments, each occurrence of R⁹ is independently —H, —N(R)₂, —OR, —CN, and optionally substituted C₁₋₆ aliphatic. In some embodiments, an occurrence of R⁹ is —N(R)₂, wherein each R is independently —H or optionally substituted C₁₋₂₀ aliphatic. In some embodiments, an occurrence of R⁹ is —N(R)₂, wherein each R is independently —H or optionally substituted C₁₋₁₀ aliphatic. In some embodiments, an occurrence of R⁹ is —NHR, wherein each R is independently —H or optionally substituted C₁₋₂₀ aliphatic. In some embodiments, an occurrence of R⁹ is —NHR, wherein each R is independently —H or optionally substituted C₁₋₁₀ aliphatic. In some embodiments, each R⁹ is independently —H, —NH₂, —CN, —CH₃. In some embodiments, an occurrence of R⁹ is optionally substituted C₁₋₆ aliphatic. In some embodiments, an occurrence of R⁹ is optionally substituted

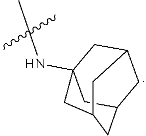 [SMILES: *NC12CC3CC(CC(C3)C1)C2]

In some embodiments, one occurrence of R⁹ is optionally substituted

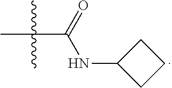 [SMILES: *C(=O)NC1CCC1]

In some embodiments, an occurrence of R⁹ is para relative to the position at which Ring A is attached to the rest of the compound. In some embodiments, each R⁹ is independently halogen, or optionally substituted C₁₋₂₀ aliphatic or C₁₋₂₀ heteroaliphatic having 1-10 heteroatoms. In some embodiments, an occurrence of R⁹ is —OCH₃.

### R¹

In some embodiments, R¹⁰ is R″ as described herein. In some embodiments, R¹⁰ is R′ as described herein.

In some embodiments, R¹⁰ is R as described herein. For example, in some embodiments, R¹⁰ is —H. In some embodiments, R¹⁰ is optionally substituted C₁₋₆ alkyl. In some embodiments, R¹⁰ is optionally substituted C₂₋₆ alkenyl. In some embodiments, R¹⁰ is optionally substituted C₂₋₆ alkynyl. In some embodiments, R¹⁰ is C₃-1₀ (e.g., 4-10, 5-10, 3, 4, 5, 6, 7, 8, 9, 10, etc. membered) cycloalkyl. In some embodiments, R¹⁰ is optionally substituted C₆₋₁₄ aryl. In some embodiments, R¹⁰ is optionally substituted C₆₋₁₀ aryl. In some embodiments, R¹⁰ is optionally substituted C₇₋₁₅ aralkyl. In some embodiments, R¹⁰ is optionally substituted 5-10 membered heteroaryl having 1-4 heteroatoms. In some embodiments, R¹⁰ is optionally substituted 3-10 membered heterocyclyl having 1-4 heteroatoms. In some embodiments, R¹⁰ is optionally substituted C₁₋₁₀ aliphatic. In some embodiments, R¹⁰ is optionally substituted C₆₋₂₀ aryl. In some embodiments, R¹⁰ is optionally substituted phenyl. In some embodiments, R¹⁰ is 2,4-dimethoxy-phenyl. In some embodiments, R¹⁰ is 3-chloro-4-methoxyphenyl.

### R¹¹

In some embodiments, R¹¹ is R″ as described herein. In some embodiments, R¹¹ is R′ as described herein.

In some embodiments, R″ is R as described herein. For example, in some embodiments, R¹¹ is —H. In some embodiments, R″ is optionally substituted C₁₋₁₀ aliphatic. In some embodiments, R¹¹ is optionally substituted C₁₋₆ aliphatic. In some embodiments, R¹¹ is optionally substituted C₁₋₆ alkyl. In some embodiments, R¹¹ is optionally substituted C₂₋₆ alkenyl. In some embodiments, R¹¹ is optionally substituted C₂₋₆ alkynyl. In some embodiments, R¹¹ is C₃₋₁₀ (e.g., 4-10, 5-10, 3, 4, 5, 6, 7, 8, 9, 10, etc. membered) cycloalkyl. In some embodiments, R¹¹ is optionally substituted C₅₋₂₀ (e.g., 5-15, 5-14, 5-10, 5-9, 5-6, 5, 6, 7, 8, 9, 10, 11, 12, 13, 14, 15, 16, 17, 18, 19, 20, etc.) aryl. In some embodiments, R¹¹ is optionally substituted C₆₋₁₄ aryl. In some embodiments, R¹¹ is optionally substituted C₆₋₁₀ aryl. In some embodiments, R¹¹ is optionally substituted phenyl. In some embodiments, R¹¹ is optionally substituted C₇_1₅ aralkyl. In some embodiments, R¹¹ is optionally substituted 5-20 (e.g., 5-15, 5-14, 5-10, 5-9, 5-6, 5, 6, 7, 8, 9, 10, 11, 12, 13, 14, 15, 16, 17, 18, 19, 20, etc.) membered heteroaryl having 1-10 (e.g., 1-9, 1-8, 1-7, 1-6, 1-5, 1-4, 1, 2, 3, 4, 5, 6, 7, 8, 9, 10, etc.) heteroatoms. In some embodiments, R¹¹ is optionally substituted 5-10 membered heteroaryl having 1-5 (e.g., 1-4, 1-3, 1, 2, 3, 4, 5, etc.) heteroatoms. In some embodiments, R¹¹ is optionally substituted 5-membered heteroaryl having 1-4 (e.g., 1-3, 1, 2, 3, 4, etc.) heteroatoms. In some embodiments, R¹¹ is optionally substituted thienyl. In some embodiments, R¹¹ is optionally substituted 3-10 (e.g., 4-10, 5-10, 3, 4, 5, 6, 7, 8, 9, 10, etc.) membered heterocyclyl having 1-4 (e.g., 1-3, 1, 2, 3, 4, etc.) heteroatoms.

### R¹²

In some embodiments, R¹² is R″ as described herein. In some embodiments, R¹² is R′ as described herein.

In some embodiments, R¹² is R as described herein. For example, in some embodiments, R¹² is —H. In some embodiments, R¹² is optionally substituted C₁₋₁₀ aliphatic. In some embodiments, R¹² is optionally substituted C₁₋₆ aliphatic. In some embodiments, R¹² is optionally substituted C₁₋₆ alkyl. In some embodiments, R¹² is optionally substituted C₂₋₆ alkenyl. In some embodiments, R¹² is optionally substituted C₂₋₆ alkynyl. In some embodiments, R¹² is C₃₋₁₀ (e.g., 4-10, 5-10, 3, 4, 5, 6, 7, 8, 9, 10, etc. membered) cycloalkyl. In some embodiments, R¹² is optionally substituted C₅₋₂₀ (e.g., 5-15, 5-14, 5-10, 5-9, 5-6, 5, 6, 7, 8, 9, 10, 11, 12, 13, 14, 15, 16, 17, 18, 19, 20, etc.) aryl. In some embodiments, R¹² is optionally substituted C₆₋₁₄ aryl. In some embodiments, R¹² is optionally substituted C₆₋₁₀ aryl. In some embodiments, R¹² is optionally substituted phenyl. In some embodiments, R¹² is optionally substituted C₇₋₁₅ aralkyl. In some embodiments, R¹² is optionally substituted 5-20 (e.g., 5-15, 5-14, 5-10, 5-9, 5-6, 5, 6, 7, 8, 9, 10, 11, 12, 13, 14, 15, 16, 17, 18, 19, 20, etc.) membered heteroaryl having 1-10 (e.g., 1-9, 1-8, 1-7, 1-6, 1-5, 1-4, 1, 2, 3, 4, 5, 6, 7, 8, 9, 10, etc.) heteroatoms. In some embodiments, R¹² is optionally substituted 5-10 membered heteroaryl having 1-5 (e.g., 1-4, 1-3, 1, 2, 3, 4, 5, etc.) heteroatoms. In some embodiments, R¹² is optionally substituted 5-membered heteroaryl having 1-4 (e.g., 1-3, 1, 2, 3, 4, etc.) heteroatoms. In some embodiments, R¹² is optionally substituted thienyl. In some embodiments, R¹² is optionally substituted 3-10 (e.g., 4-10, 5-10, 3, 4, 5, 6, 7, 8, 9, 10, etc.) membered heterocyclyl having 1-4 (e.g., 1-3, 1, 2, 3, 4, etc.) heteroatoms.

In some embodiments, R″ and R¹² are each independently hydrogen, or an optionally substituted group selected from C₁₋₂₀ (e.g., C₁₋₁₅, C₁₋₁₀, C₁₋₉, C₁₋₈, C₁₋₆, C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, etc.) aliphatic, C₁₋₂₀ (e.g., C₁₋₁₅, C₁₋₁₀, C₁₋₉, C₁₋₈, C₁₋₆, C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, etc.) heteroaliphatic having 1-10 (e.g., 1-9, 1-8, 1-7, 1-6, 1-5, 1-4, 1, 2, 3, 4, 5, 6, 7, 8, 9, 10, etc.) heteroatoms, C₃₋₂₀ (e.g., 3-15, 3-10, 4-20, 5-20, 3, 4, 5, 6, 7, 8, 9, 10, 11, 12, 13, 14, 15, 16, 17, 18, 19, 20, etc. membered) heterocyclyl having 1-10 (e.g., 1-9, 1-8, 1-7, 1-6, 1-5, 1-4, 1, 2, 3, 4, 5, 6, 7, 8, 9, 10, etc.) heteroatoms, C₅₋₂₀ (e.g., 5-15, 5-14, 5-10, 5, 6, 7, 8, 9, 10, 11, 12, 13, 14, 15, 16, 17, 18, 19, 20, etc. membered) aryl, and 5-20 (e.g., 5-15, 5-14, 5-10, 5, 6, 7, 8, 9, 10, 11, 12, 13, 14, 15, 16, 17, 18, 19, 20, etc.) membered heteroaryl having 1-10 (e.g., 1-9, 1-8, 1-7, 1-6, 1-5, 1-4, 1, 2, 3, 4, 5, 6, 7, 8, 9, 10, etc.) heteroatoms; or R″ and R¹² are taken together with the carbon atom to which they are attached to form an optionally substituted C₃-1₀ (e.g., 4-10, 5-10, 3, 4, 5, 6, 7, 8, 9, 10, etc.) non-aromatic unit having 0-5 (e.g., 1-5, 1-4, 1-3, 0, 1, 2, 3, 4, 5, etc.) heteroatoms.

In some embodiments, R″ and R¹² are both R, and are taken together with the carbon atom to which they are attached to form a ring as described herein. In some embodiments, R″ and R¹² are taken together with the carbon atom to which they are attached to form an optionally substituted 3-20 (e.g., 3-15, 3-10, 4-20, 5-20, 3, 4, 5, 6, 7, 8, 9, 10, 11, 12, 13, 14, 15, 16, 17, 18, 19, 20, etc.) membered ring having 0-5 (e.g., 1-5, 1-4, 1-3, 0, 1, 2, 3, 4, 5, etc.) heteroatoms. In some embodiments, R″ and R¹² are taken together with the carbon atom to which they are attached to form an optionally substituted 3-10 (e.g., 4-10, 5-10, 3, 4, 5, 6, 7, 8, 9, 10, etc.) membered ring having 0, 1, 2, or 3 heteroatoms. In some embodiments, R¹ and R² are taken together with the carbon atom to which they are attached to form an optionally substituted 3-14 (e.g., 3-10, 3-9, 5-14, 3, 4, 5, 6, 7, 8, 9, 10, 11, 12, 13, 14, etc.) membered cycloaliphatic ring. In some embodiments, R¹¹ and R¹² are taken together with the carbon atom to which they are attached to form an optionally substituted 3-10 (e.g., 4-10, 5-10, 3, 4, 5, 6, 7, 8, 9, 10, etc.) membered cycloalkyl ring. In some embodiments, R¹¹ and R¹² are taken together with the carbon atom to which they are attached to form an optionally substituted 3-14 (e.g., 3-10, 3-9, 5-14, 3, 4, 5, 6, 7, 8, 9, 10, 11, 12, 13, 14, etc.) membered heterocyclyl ring having 1, 2 or 3 heteroatoms.

### R¹³

In some embodiments, R¹³ is R″ as described herein. In some embodiments, R¹³ is R′ as described herein. In some embodiments, R¹³ is R as described herein. In some embodiments, R¹³ is —H.

In some embodiments, R¹³ is optionally substituted C₁₋₁₀ aliphatic. In some embodiments, R¹³ is optionally substituted C₁₋₆ aliphatic. In some embodiments, R¹³ is optionally substituted C₁₋₆ alkyl.

In some embodiments, R¹³ is -L-R′ wherein each of L and R′ is independently as described herein. In some embodiments, L is optionally substituted bivalent linear or branched C₁₋₆ alkylene. In some embodiments, L is optionally substituted bivalent linear C₁₋₆ alkylene. In some embodiments, L is optionally substituted —CH₂—. In some embodiments, L is —CH₂—. In some embodiments, L is optionally substituted —CH₂—CH₂—. In some embodiments, L is —CH₂—CH₂—. In some embodiments, R′ is R as described herein. In some embodiments, R′ is not —H. In some embodiments, R′ is optionally substituted aryl. In some embodiments, R′ is optionally substituted phenyl. In some embodiments, R′ is —C(O)OR. In some embodiments, R′ is —C(O)OEt. In some embodiments, R¹³ is optionally substituted cycloalkylC₀₋₄alkyl. In some embodiments, R¹³ is optionally substituted arylC₀₋₄alkyl. In some embodiments, R¹³ is optionally substituted heteroarylC₀₋₄alkyl. In some embodiments, R¹³ is optionally substituted aryl-S(O)₂C₀₋₄alkyl.

### R¹⁴

In some embodiments, R¹⁴ is R″ as described herein. In some embodiments, R¹⁴ is R′ as described herein. In some embodiments, R¹⁴ is R as described herein. In some embodiments, R¹⁴ is —H.

In some embodiments, R¹⁴ is optionally substituted C₁₋₁₀ aliphatic. In some embodiments, R¹⁴ is optionally substituted C₁₋₆ aliphatic. In some embodiments, R¹⁴ is optionally substituted C₁₋₆ alkyl.

In some embodiments, R¹⁴ is -L-R′ wherein each of L and R′ is independently as described herein. In some embodiments, L is optionally substituted bivalent linear or branched C₁₋₆ alkylene. In some embodiments, L is optionally substituted bivalent linear C₁₋₆ alkylene. In some embodiments, L is optionally substituted —CH₂—. In some embodiments, L is —CH₂—. In some embodiments, L is optionally substituted —CH₂—CH₂—. In some embodiments, L is —CH₂—CH₂—. In some embodiments, R′ is R as described herein. In some embodiments, R′ is not —H. In some embodiments, R′ is optionally substituted aryl. In some embodiments, R′ is optionally substituted phenyl. In some embodiments, R′ is —C(O)OR. In some embodiments, R′ is —C(O)OEt. In some embodiments, R¹⁴ is optionally substituted cycloalkylC₀₋₄alkyl. In some embodiments, R¹⁴ is optionally substituted arylC₀₋₄alkyl. In some embodiments, R¹⁴ is optionally substituted heteroarylC₀₋₄alkyl. In some embodiments, R¹⁴ is optionally substituted aryl-S(O)₂C₀₋₄alkyl.

### R¹⁵

In some embodiments, R¹⁵ is -L-R′ wherein each of L and R′ is independently as described herein. In some embodiments, L is optionally substituted bivalent C₁₋₆ aliphatic, wherein a methylene units is replaced with -Cy- as described herein. In some embodiments, -Cy- is optionally substituted phenylene. In some embodiments, -Cy- is optionally substituted 1,2-phenylene. In some embodiments, L is optionally substituted bivalent C₁₋₆ aliphatic, wherein a methylene units is replaced with —O—. In some embodiments, L is optionally substituted —CH₂-(1,2-phenylene)-O—, wherein the —CH₂— is bonded to the nitrogen atom. In some embodiments, R¹⁵ is -L-R′, wherein L is optionally substituted C₁₋₆ alkylene and R′ is as described herein. In some embodiments, R″ is -L-R′, wherein L is —CH₂—C(O)N(R′)—, wherein the —CH₂— is optionally substituted and is bonded to the nitrogen atom, and each R′ is independently as described herein. In some embodiments, R″ is -L-R′, wherein L is —C(R′)₂—C(O)N(R′)—, wherein the —C(R′)₂— is bonded to the nitrogen atom, and each R′ is independently as described herein.

In some embodiments, L is -L″-L^{x}-L^{y}-L^{z}-, wherein L″ is a covalent bond or an optionally substituted bivalent C₁₋₇ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—; and each of L^{x}, L^{y} and L^{z} is independently a covalent bond or optionally substituted methylene which is optionally replaced with —C(R′)—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{y} is a covalent bond, —N(R′), or —O—. In some embodiments, L^{y} is a covalent bond, —C(O)—, —C(S)—, —C(NR′)—, —C(O)N(R′)—, —C(S)N(R′)—, or —C(NR′)(NR′)—. In some embodiments, L^{z} is a covalent bond, —N(R′)—, —N(R′)O— or —O—. In some embodiments, L^{x} is a covalent bond. In some embodiments, L^{x} is —N(R′)— wherein R′ is as described herein. In some embodiments, L^{x} is —O—. In some embodiments, Ly is a covalent bond. In some embodiments, L^{y} is —C(O)—. In some embodiments, L^{y} is —C(S)—. In some embodiments, L^{y} is —C(NR′)— wherein R′ is as described herein. In some embodiments, L^{y} is —C(O)N(R′)— wherein R′ is as described herein. In some embodiments, L^{y} is —C(S)N(R′)— wherein R′ is as described herein. In some embodiments, L^{y} is —C(NR′)(NR′)— wherein each R′ is independently as described herein. In some embodiments, L^{z} is a covalent bond. In some embodiments, L^{z} is —N(R′)— wherein R′ is as described herein. In some embodiments, L^{z} is —N(R′)O— wherein R′ is as described herein. In some embodiments, L^{z} is —O—.

In some embodiments, L″ is bonded to the nitrogen atom.

In some embodiments, R¹⁵ is R′ as described herein. In some embodiments, R¹⁵ is R as described herein. In some embodiments, R¹⁵ is —H.

In some embodiments, R¹⁵ is an optionally substituted group selected from C₁₋₁₀ aliphatic, C₁₋₂₀ heteroaliphatic having 1-10 heteroatoms, C₃₋₂₀ heterocyclyl having 1-10 heteroatoms, C₅₋₂₀ aryl, 5-20 membered heteroaryl having 1-10 heteroatoms, C₀₋₁₀ aliphatic-C₃₋₂₀cycloalkyl, C₀₋₁₀ aliphatic-C₃₋₂₀ heterocyclyl having 1-10 heteroatoms, C₀₋₁₀ aliphatic-C₅₋₂₀ aryl, C₀₋₁₀ aliphatic-5-20 membered heteroaryl. In some embodiments, R¹⁵ is optionally substituted C₁₋₁₀ (e.g., 1-9, 1-8, 1-7, 1-6, 1-5, 1-4, 1, 2, 3, 4, 5, 6, 7, 8, 9, 10, etc.) aliphatic. In some embodiments, R¹⁵ is optionally substituted C₁₋₆ alkyl. In some embodiments, R¹⁵ is methyl. In some embodiments, R¹⁵ is octyl. In some embodiments, R¹⁵ is optionally substituted C₁₋₂aliphatic-C₅₋₂₀aryl. In some embodiments, R¹⁵ is optionally substituted C₅₋₂₀ aryl. In some embodiments, R¹⁵ is optionally substituted 6-14 membered aryl. In some embodiments, R¹⁵ is optionally substituted 6-10 membered aryl. In some embodiments, R¹⁵ is optionally substituted phenyl. In some embodiments, R¹⁵ is optionally substituted 5-20 (e.g., 5-15, 5-14, 5-10, 5-9, 5-6, 5, 6, 7, 8, 9, 10, 11, 12, 13, 14, 15, 16, 17, 18, 19, 20, etc.) membered heteroaryl having 1-10 (e.g., 1-9, 1-8, 1-7, 1-6, 1-5, 1-4, 1, 2, 3, 4, 5, 6, 7, 8, 9, 10, etc.) heteroatoms. In some embodiments, R¹⁵ is 3-cyanophenyl, 4-cyanophenyl, 3-chlorophenyl, 4-chlorophenyl, 3-fluorophenyl, 3-hydroxyphenyl, 3-methoxyphenyl, 4-methoxyphenyl, 3-methoxycarbonylphenyl, 4-methoxycarbonylphenyl, 2,3-dichlorophenyl, 2,5-dichlorophenyl, 3,4-dichlorophenyl, 3-chloro-2-fluorophenyl, 5-chloro-2-fluorophenyl, 5-chloro-2-iodophenyl, 3-chloro-4-methoxyphenyl, 2,4-dimethoxyphenyl, 3-methylsulfonylphenyl, 4-methylsulfonylphenyl, 3-(pyrazol-3-yl)phenyl, 3-(1-methylpyrazol-3-yl)phenyl, 2-morpholinophenyl, 4-dimethylaminophenyl, or 1-oxo-2,3-dihydro-1H-inden-4-yl. In some embodiments, R¹⁵ is optionally substituted indazolyl, chloropyridinyl, indolyl, methylindolyl, indazolyl, methylindazolyl, methylbenzo[d]imidazolyl, benzo[d]thiazolyl, benzo[c][1,2,5]oxadiazolyl, benzo[c][1,2,5]thiadiazolyl, benzo[d]thiazolyl, 1H-indazolyl, or [1,2,4]triazolo[4,3-a]pyridinyl. In some embodiments, R¹⁵ is 5-chloropyridin-3-yl, indol-5-yl, 1-mtehylindol-5-yl, indazol-5-yl, 1-methylindazol-7-yl, 1-methylbenzo[d]imidazol-5-yl, 1-methylbenzo[d]imidazol-6-yl, benzo[d]thiazol-5-yl, benzo[c][1,2,5]oxadiazol-4-yl, benzo[c][1,2,5]thiadiazol-4-yl, benzo[c][1,2,5]thiadiazol-5-yl, benzo[d]thiazol-5-yl, benzo[d]thiazol-6-yl, 1-methyl-1H-indazol-5-yl, 1-methyl-1H-indazol-6-yl, [1,2,4]triazolo[4,3-a]pyridin-6-yl, [1,2,4]triazolo[4,3-a]pyridin-7-yl, or [1,2,4]triazolo[4,3-a]pyridin-8-yl. In some embodiments, R¹⁵ is optionally substituted C₃₂₀ (e.g., 3-15, 3-10, 4-20, 5-20, 3, 4, 5, 6, 7, 8, 9, 10, 11, 12, 13, 14, 15, 16, 17, 18, 19, 20, etc.) heterocyclyl having 1-10 (e.g., 1-9, 1-8, 1-7, 1-6, 1-5, 1-4, 1, 2, 3, 4, 5, 6, 7, 8, 9, 10, etc.) heteroatoms. In some embodiments, R⁵ is benzo[d][1,3]dioxole-5-yl, 3-oxo-1,3-dihydroisobenzofuran-5-yl, benzo[d][1,3]dioxol-4-yl, 6 iodobenzo[d][1,3]dioxol-5-yl, or 2,2-difluorobenzo[d][1,3]dioxol-5-yl.

### R¹⁶

In some embodiments, R¹⁶ is -L-R′ wherein each of L and R′ is independently as described herein. In some embodiments, L is optionally substituted bivalent C₁₋₆ aliphatic, wherein a methylene units is replaced with -Cy- as described herein. In some embodiments, -Cy- is optionally substituted phenylene. In some embodiments, -Cy- is optionally substituted 1,2-phenylene. In some embodiments, L is optionally substituted bivalent C₁₋₆ aliphatic, wherein a methylene units is replaced with —O—. In some embodiments, L is optionally substituted —CH₂-(1,2-phenylene)-O—, wherein the —CH₂— is bonded to the nitrogen atom. In some embodiments, R¹⁶ is -L-R′, wherein L is optionally substituted C₁₋₆ alkylene and R′ is as described herein. In some embodiments, R¹⁶ is -L-R′, wherein L is —CH₂—C(O)N(R′)—, wherein the —CH₂— is optionally substituted and is bonded to the nitrogen atom, and each R′ is independently as described herein. In some embodiments, R¹⁶ is -L-R′, wherein L is —C(R′)₂—C(O)N(R′)—, wherein the —C(R′)₂— is bonded to the nitrogen atom, and each R′ is independently as described herein.

In some embodiments, L is -L″-L^{x}-L^{y}-L^{z}-, wherein L″ is a covalent bond or an optionally substituted bivalent C₁₇ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—; and each of L^{x}, L and L^{Z} is independently a covalent bond or optionally substituted methylene which is optionally replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{x} is a covalent bond, —N(R′), or —O—. In some embodiments, L^{y} is a covalent bond, —C(O)—, —C(S)—, —C(NR′)—, —C(O)N(R′)—, —C(S)N(R′)—, or —C(NR′)(NR′)—. In some embodiments, L^{z} is a covalent bond, —N(R′)—, —N(R′)O— or —O—. In some embodiments, U is a covalent bond. In some embodiments, U is —N(R′)— wherein R′ is as described herein. In some embodiments, U is —O—. In some embodiments, L^{y} is a covalent bond. In some embodiments, L^{y} is —C(O)—. In some embodiments, L^{y} is —C(S)—. In some embodiments, L^{y} is —C(NR′)— wherein R′ is as described herein. In some embodiments, L^{y} is —C(O)N(R′)— wherein R′ is as described herein. In some embodiments, L^{y} is —C(S)N(R′)— wherein R′ is as described herein. In some embodiments, L^{y} is —C(NR′)(NR′)— wherein each R′ is independently as described herein. In some embodiments, L^{z} is a covalent bond. In some embodiments, L^{z} is —N(R′)— wherein R′ is as described herein. In some embodiments, L^{z} is —N(R′)O— wherein R′ is as described herein. In some embodiments, L^{z} is —O—.

In some embodiments, L″ is bonded to the nitrogen atom.

In some embodiments, R¹⁶ is R′ as described herein. In some embodiments, R¹⁶ is R as described herein. In some embodiments, R¹⁶ is —H.

In some embodiments, R¹⁶ is an optionally substituted group selected from C₁₋₁₀ aliphatic, C₁₋₂₀ heteroaliphatic having 1-10 heteroatoms, C₃₋₂₀ heterocyclyl having 1-10 heteroatoms, C₅₋₂₀ aryl, 5-20 membered heteroaryl having 1-10 heteroatoms, C₀₋₁₀ aliphatic- C₃₋₂₀cycloalkyl, C₀₋₁₀ aliphatic-C₃₋₂₀ heterocyclyl having 1-10 heteroatoms, C₀₋₁₀ aliphatic-C₅₋₂₀ aryl, C₀₋₁₀ aliphatic-5-20 membered heteroaryl. In some embodiments, R¹⁶ is optionally substituted C₁₋₁₀ (e.g., 1-9, 1-8, 1-7, 1-6, 1-5, 1-4, 1, 2, 3, 4, 5, 6, 7, 8, 9, 10, etc.) aliphatic. In some embodiments, R¹⁶ is optionally substituted C₁₋₁₀ alkyl. In some embodiments, R¹⁶ is optionally substituted C₁₋₆ alkyl. In some embodiments, R¹⁶ is methyl. In some embodiments, R¹⁶ is octyl. In some embodiments, R¹⁶ is optionally substituted arylC₀₋₄alkyl. In some embodiments, R¹⁶ is optionally substituted C₁₋₂aliphatic-C₅₋₂₀aryl. In some embodiments, R¹⁶ is optionally substituted C₅₋₂₀ aryl. In some embodiments, R¹⁶ is optionally substituted 6-14 membered aryl. In some embodiments, R¹⁶ is optionally substituted 6-10 membered aryl. In some embodiments, R¹⁶ is optionally substituted phenyl. In some embodiments, R¹⁶ is optionally substituted 5-20 (e.g., 5-15, 5-14, 5-10, 5-9, 5-6, 5, 6, 7, 8, 9, 10, 11, 12, 13, 14, 15, 16, 17, 18, 19, 20, etc.) membered heteroaryl having 1-10 (e.g., 1-9, 1-8, 1-7, 1-6, 1-5, 1-4, 1, 2, 3, 4, 5, 6, 7, 8, 9, 10, etc.) heteroatoms. In some embodiments, R¹⁶ is optionally substituted C₃₋₁₄ (e.g., 3-10, 4-14, 5-10, 3, 4, 5, 6, 7, 8, 9, 10, 11, 12, 13, 14, etc.) cycloaliphatic. In some embodiments, wherein R¹⁶ is optionally substituted 3-20 (e.g., 3-15, 3-10, 4-20, 5-20, 3, 4, 5, 6, 7, 8, 9, 10, 11, 12, 13, 14, 15, 16, 17, 18, 19, 20, etc.) membered heterocyclyl having 1-6 (e.g., 1-5, 1-4, 1-3, 1, 2, 3, 4, 5, 6, etc.) heteroatoms. In some embodiments, R¹⁶ is optionally substituted phenyl, thienyl, thiazolyl, tetrahydropyranyl or cyclohexyl. In some embodiments, R¹⁶ is 4-chloro-benzenyl, 4-methoxy-benzenyl, benzyl, 4-hydroxy-benzenyl, 3-cyano-benzenyl, 3-fluoro-benzenyl, 3-hydroxy-benzenyl, 4-cyano-benzenyl, 4-fluoro-benzenyl, methyl, cyclopropylmethyl, cyclopropyl, cyclobutyl, cyclopentyl, cyclohexyl, 4-methoxycarbonylcyclohexyl, phenyl, 3-aminophenyl, 4-aminophenyl, 2-fluorophenyl, 4-fluorophenyl, 3-nitrophenyl, 4-nitrophenyl, 1-methoxycarbonylmethoxyphenyl, 3-(2-hydroxyethyl)phenyl, 4-(2-hydroxyacetamido)phenyl, 2-thienyl, 5-thiazolyl, 4-pyridyl, or tetrahydro-2H-pyran-4-yl. In some embodiments, R¹⁶ is 3-methylphenyl. In some embodiments, R¹⁶ is

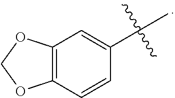 [SMILES: *c1ccc2c(c1)OCO2]

In some embodiments, R¹⁶ is 3-indolyl. In some embodiments, R¹⁶ is

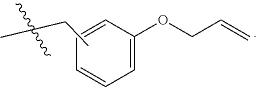

In some embodiments, R¹⁵ and R¹⁶ are R, and are taken together with the atom to which they are attached to form an optionally substituted ring as described herein. In some embodiments, R¹⁵ and R¹⁶ are taken together with the nitrogen atom to which they are attached to form an optionally substituted 3-10 (e.g., 4-10, 5-10, 3, 4, 5, 6, 7, 8, 9, 10, etc.) membered saturated or saturated ring having 0-4 (e.g., 1-4, 1-3, 0, 1, 2, 3, 4, etc.) heteroatoms in addition to the nitrogen atom to which they are attached. In some embodiments, R¹⁵ and R¹⁶ are taken together with the nitrogen atom to which they are attached to form an optionally substituted 3-10 membered saturated or partially unsaturated ring having 0-4 heteroatoms in addition to the nitrogen atom to which they are attached.

### R¹⁷

In some embodiments, R¹⁷ is an optionally substituted 5-20 (e.g., 5-15, 5-14, 5-10, 5-9, 5-6, 5, 6, 7, 8, 9, 10, 11, 12, 13, 14, 15, 16, 17, 18, 19, 20, etc.) membered, monocyclic, bicyclic or polycyclic aromatic ring having 0-5 heteroatoms. In some embodiments, R¹⁷ is optionally substituted 5-6 membered heteroaryl having 1-4 (e.g., 1-3, 1, 2, 3, 4, etc.) heteroatoms. In some embodiments, R¹⁷ is optionally substituted phenyl. In some embodiments, R¹⁷ is an optionally substituted 8-10 membered bicyclic aromatic ring having 0-4 (e.g., 1-4, 1-3, 0, 1, 2, 3, 4, etc.) heteroatoms. In some embodiments, R¹⁷ is substituted. In some embodiments, one or more substituents are independently halogen. In some embodiments, one or more substituents are independently —F or —Cl. In some embodiments, each substituent is independently —F or —Cl. In some embodiments, there are 1 or 2 substituents. In some embodiments, R¹⁷ is phenyl optionally substituted with 1, 2, or 3 substituents each independently of which is independently —F or —Cl. In some embodiments, R¹⁷ is unsubstituted.

### R¹⁸

In some embodiments, R¹⁸ is an optionally substituted 5-20 (e.g., 5-15, 5-14, 5-10, 5-9, 5-6, 5, 6, 7, 8, 9, 10, 11, 12, 13, 14, 15, 16, 17, 18, 19, 20, etc.) membered, monocyclic, bicyclic or polycyclic aromatic ring having 0-5 heteroatoms. In some embodiments, R¹⁸ is optionally substituted 5-6 membered heteroaryl having 1-4 (e.g., 1-3, 1, 2, 3, 4, etc.) heteroatoms. In some embodiments, R¹⁸ is optionally substituted phenyl. In some embodiments, R^{x} is an optionally substituted 8-10 membered bicyclic aromatic ring having 0-4 (e.g., 1-4, 1-3, 0, 1, 2, 3, 4, etc.) heteroatoms. In some embodiments, R¹⁸ is substituted. In some embodiments, one or more substituents are independently halogen. In some embodiments, one or more substituents are independently —F or —Cl. In some embodiments, each substituent is independently —F or —Cl. In some embodiments, there are 1 or 2 substituents. In some embodiments, R″ is phenyl optionally substituted with 1, 2, or 3 substituents each independently of which is independently —F or —Cl. In some embodiments, R¹⁸ is unsubstituted.

### L^{A}

As described herein, L^{A} is L as described herein. In some embodiments, L^{A} is a covalent bond. In some embodiments, L^{A} is an optionally substituted bivalent C₁₋₁₀ (e.g., C₁₋₆, C₁, C₂, C₃, C₄, C₅, C₆, C₇, C₈, C₉, C₁₀, etc.) aliphatic or heteroaliphatic having 1-6 (e.g., 1, 2, 3, 4, 5, or 6) heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{A} is an optionally substituted bivalent C₁₋₁₀ (e.g., C₁₋₆, C₁, C₂, C₃, C₄, C₅, C₆, C₇, C₈, C₉, C₁₀, etc.) aliphatic wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{A} is an optionally substituted bivalent C₁₋₆(e.g., C₁, C₂, C₃, C₄, C₅, or C₆) aliphatic wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{A} is an optionally substituted bivalent C₁₄ aliphatic wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{A} is an optionally substituted bivalent C₁₋₂ aliphatic wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, L^{A} is an optionally substituted bivalent C₁₋₁₀ (e.g., C₁₋₆, C₁, C₂, C₃, C₄, C₅, C₆, C₇, C₈, C₉, C₁₈, etc.) aliphatic. In some embodiments, the bivalent aliphatic is linear. In some embodiments, it is branched. In some embodiments, it is saturated. In some embodiments, it is partially unsaturated. In some embodiments, L^{A} is or comprises optionally substituted —CH₂—. In some embodiments, L^{A} is or comprises —CH₂—. In some embodiments, L^{A} is or comprises -Cy- as described herein. For example, in some embodiments, L^{A} is optionally substituted phenylene. In some embodiments, L^{A} is optionally substituted 1,4-phenylene.

### Ring

As described herein, various groups or moieties in the present disclosure are or comprising rings, e.g., Ring A, Ring C, Ring P, -Cy-, rings formed by two or more R groups (and/or groups that can be R such as R′, R″, R^{s}, R^{w1}, R^{w2}, R^{w3}, etc.) taken together with their intervening atom(s). Unless otherwise specified, rings are optionally substituted. Certain rings are described herein as examples.

In some embodiments, a ring is an optionally substituted 3-20 (e.g., 5, 6, 7, 8, 9, 10, 11, 12, 13, 14, 15, 16, 17, 18, 19, or 20) membered, monocyclic, bicyclic or polycyclic ring having 0-5 (e.g., 0, 1, 2, 3, 4 or 5) heteroatoms. In some embodiments, a ring is monocyclic. In some embodiments, A ring is bicyclic. In some embodiments, A ring is polycyclic. In some embodiments, A ring is saturated. In some embodiments, A ring is partially unsaturated. In some embodiments, A ring is aromatic. In some embodiments, A ring is 3-membered. In some embodiments, it is 4-membered. In some embodiments, it is 5-membered. In some embodiments, it is 6-membered. In some embodiments, it is 7-membered. In some embodiments, it is 8-membered. In some embodiments, it is 9-membered. In some embodiments, it is 10-membered. In some embodiments, it is 11-membered. In some embodiments, it is 12-membered. In some embodiments, it is 13-membered. In some embodiments, it is 14-membered. In some embodiments, it is 15-membered. In some embodiments, it is 16-membered. In some embodiments, it is 17-membered. In some embodiments, it is 18-membered. In some embodiments, it is 19-membered. In some embodiments, it is 20-membered. In some embodiments, a ring is bicyclic or polycyclic comprising two or more monocyclic ring unit. For example, a indole ring is bicyclic and has two monocyclic ring units one of which is 5-membered and the other is 6-membered. In some embodiments, each monocyclic ring unit is independently an optionally substituted, 3-10, 3-8, 3-7, 3-6, 4-10, 5-10, 5-9, 5-8, 5-7, 5-6, 3, 4, 5, 6, 7, 8, 9, or 10 membered, saturated, partially unsaturated or aromatic ring having 0-4 (e.g., 0, 1, 2, 3 or 4) heteroatoms. In some embodiments, each monocyclic ring unit is independently 3-7 membered. In some embodiments, a monocyclic ring unit is 3-membered. In some embodiments, a monocyclic ring unit is 4-membered. In some embodiments, a monocyclic ring unit is 5-membered. In some embodiments, a monocyclic ring unit is 6-membered. In some embodiments, a monocyclic ring unit is 7-membered. In some embodiments, a monocyclic ring unit is 8-membered. In some embodiments, a monocyclic ring unit is 9-membered. In some embodiments, a monocyclic ring unit is 10-membered. In some embodiments, as described herein, each heteroatom is independently selected nitrogen, oxygen and sulfur. In some embodiments, a monocyclic ring unit is saturated. In some embodiments, a monocyclic ring unit is partially unsaturated. In some embodiments, a monocyclic ring unit is aromatic. In some embodiments, a monocyclic ring unit is heteroaromatic. In some embodiments, there is one ring atom of a monocyclic ring unit that is a heteroatom. In some embodiments, there are two or more ring atoms of a monocyclic ring unit each of which is independently a heteroatom. In some embodiments, there are three or more ring atoms of a monocyclic ring unit each of which is independently a heteroatom. In some embodiments, there are four or more ring atoms of a monocyclic ring unit each of which is independently a heteroatom.

In some embodiments, a ring or a monocyclic unit thereof is an optionally substituted 3-20 (e.g., 3-15, 3-10, 3-9, 3-8, 3-7, 3-6, 1, 2, 3, 4, 5, 6, 7, 8, 9, 10, 11, 12, 13, 14, 15, 16, 17, 18, 19, 20 etc.) cycloaliphatic ring. In some embodiments, a ring or a monocyclic unit thereof is an optionally substituted 3-20 (e.g., 3-15, 3-10, 3-9, 3-8, 3-7, 3-6, 1, 2, 3, 4, 5, 6, 7, 8, 9, 10, 11, 12, 13, 14, 15, 16, 17, 18, 19, 20 etc.) cycloalkyl ring. In some embodiments, a ring or a monocyclic unit thereof is an optionally substituted 3-20 (e.g., 3-15, 3-10, 3-9, 3-8, 3-7, 3-6, 1, 2, 3, 4, 5, 6, 7, 8, 9, 10, 11, 12, 13, 14, 15, 16, 17, 18, 19, 20 etc.) heteroaliphatic ring having 1-6 (e.g., 1, 2, 3, 4, 5, or 6, etc.) heteroatoms. In some embodiments, a ring or a monocyclic unit thereof is an optionally substituted 3-20 (e.g., 3-15, 3-10, 3-9, 3-8, 3-7, 3-6, 1, 2, 3, 4, 5, 6, 7, 8, 9, 10, 11, 12, 13, 14, 15, 16, 17, 18, 19, 20 etc.) heterocyclyl ring having 1-6 (e.g., 1, 2, 3, 4, 5, or 6, etc.) heteroatoms. In some embodiments, a ring or a monocyclic unit thereof is an optionally substituted phenyl ring. In some embodiments, a ring or a monocyclic unit thereof is an optionally substituted 5-membered heteroaryl ring having 1-4 heteroatoms. In some embodiments, a ring or a monocyclic unit thereof is an optionally substituted 6-membered heteroaryl ring having 1-4 heteroatoms. In some embodiments, at least one heteroatom is nitrogen. In some embodiments, each heteroatom is independently selected from nitrogen, oxygen and sulfur.

### Ring A

In some embodiments, Ring A is an optionally substituted, 3-20 (e.g., 3-15, 3-10, 4-20, 5-20, 3, 4, 5, 6, 7, 8, 9, 10, 11, 12, 13, 14, 15, 16, 17, 18, 19, 20, etc.) membered, monocyclic, bicyclic or polycyclic ring having 0-10 (e.g., 1-10, 1-9, 1-8, 1-7, 1-6, 1-5, 1-4, 0, 1, 2, 3, 4, 5, 6, 7, 8, 9, 10, etc.) heteroatoms. In some embodiments, Ring A is an optionally substituted ring as described herein.

In some embodiments, Ring A is an optionally substituted aromatic ring having 0-6 (e.g., 1-6, 1-4, 1-3, 0, 1, 2, 3, 4, 5, 6, etc.) heteroatoms. In some embodiments, Ring A is or comprises an optionally substituted monocyclic aromatic ring having 0-4 (e.g., 1-4, 1-3, 0, 1, 2, 3, 4, etc.) heteroatoms. In some embodiments, Ring A is optionally substituted phenyl. In some embodiments, Ring A is optionally substituted pyridyl. In some embodiments, Ring A is optionally substituted pyrazolyl. In some embodiments, Ring A is phenyl. In some embodiments, Ring A is optionally substituted tetrahydro-2H-pyranyl. In some embodiments, Ring A is tetrahydro-2H-pyranyl. In some embodiments, Ring A is or comprises an optionally substituted monocyclic non-aromatic ring unit having 0-6 heteroatoms. In some embodiments, Ring A is optionally substituted piperidyl. In some embodiments, Ring A is optionally substituted

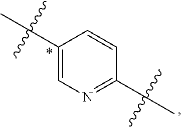 [SMILES: *c1ccc(*)nc1]

wherein “*” indicates the atom bonded to L^{A}. In some embodiments, Ring A is optionally substituted

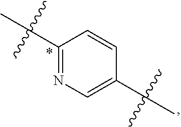 [SMILES: *c1ccc(*)nc1]

wherein “*” indicates the atom bonded to L^{A}. In some embodiments, Ring A is optionally substituted

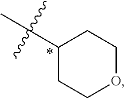 [SMILES: *C1CCOCC1]

wherein “*” indicates the atom bonded to L^{A}. In some embodiments, Ring A is

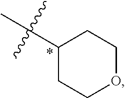 [SMILES: *C1CCOCC1]

wherein “*” indicates the atom bonded to L^{A}.

In some embodiments, Ring A is bonded to one or more occurrences of R^{s}, each of which is independently as described herein. In some embodiments, an occurrence of R^{s} is optionally substituted C₁₋₂₀ aliphatic. In some embodiments, an occurrence of R^{s} is optionally substituted C₁₋₂₀ heteroaliphatic having 1-10 heteroatoms. In some embodiments, an occurrence of R^{s} is optionally substituted heterocyclylC₀₋₄alkyl. In some embodiments, an occurrence of R^{s} is halogen. In some embodiments, an occurrence of R^{s}—C(O)N(R′)₂ wherein each R′ is independently as described herein. In some embodiments, an occurrence of R^{s} is —S(O)N(R′) wherein R′ is as described herein. In some embodiments, an occurrence of R^{s} is —S(O)₂N(R′)₂ wherein each R′ is independently as described herein. In some embodiments, an occurrence of R^{s} is optionally substituted

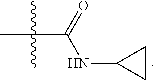 [SMILES: *C(=O)NC1CC1]

In some embodiments, an occurrence of R′ is optionally substituted

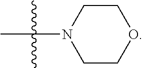 [SMILES: *N1CCOCC1]

In some embodiments, Ring A is an optionally substituted 3-10 (e.g., 4-10, 5-10, 3, 4, 5, 6, 7, 8, 9, 10, etc.) membered saturated ring having 0-4 (e.g., 1-4, 1-3, 0, 1, 2, 3, 4, etc.) heteroatoms.

### Ring B

In some embodiments, Ring B is an optionally substituted, 3-20 (e.g., 3-15, 3-10, 4-20, 5-20, 3, 4, 5, 6, 7, 8, 9, 10, 11, 12, 13, 14, 15, 16, 17, 18, 19, 20, etc.) membered, monocyclic, bicyclic or polycyclic ring having 0-6 (e.g., 1-6, 1-5, 1-4, 0, 1, 2, 3, 4, 5, 6, etc.) heteroatoms. In some embodiments, Ring B is an optionally substituted ring described herein. In some embodiments, Ring B is an optionally substituted 3-8 (e.g., 4-8, 5-8, 3, 4, 5, 6, 7, 8, 9, 10, etc.) membered monocyclic ring having 0-6 (e.g., 1-6, 1-5, 1-4, 1-3, 0, 1, 2, 3, 4, 5, 6, etc.) heteroatoms. In some embodiments, Ring B is optionally substituted 5-6 membered partially unsaturated ring having 1, 2, or 3 heteroatoms. In some embodiments, Ring B is an optionally substituted 5-membered partially unsaturated ring having 1, 2, or 3 heteroatoms. In some embodiments, Ring B is

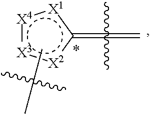

wherein “*” indicates the atom bonded to

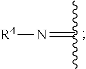

each of X¹, X², X³ and X⁴ is independently —O—, —S—, —N(—)—, —C(—)═, or optionally substituted —CH(—)—, —CH═ or —NH—, wherein at least one of X¹, X², X³ and X⁴ is —N(—)—, —C(—)═, or optionally substituted —CH(—)—. In some embodiments, Ring B is

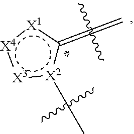

wherein “*” indicates the atom bonded to

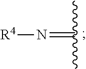

each of X¹, X³ and X⁴ is independently —O—, —S—, or optionally substituted —CH═ or —NH—, and X² is —N(—)—, —C(—)═, or optionally substituted —CH(—)—.

In some embodiments, X¹ In some embodiments, —O—. In some embodiments, X¹ is —S—. In some embodiments, X¹ is —N(—)—. In some embodiments, X¹ is —C(—)═. In some embodiments, X¹ is optionally substituted —CH(—)—. In some embodiments, X¹ is optionally substituted —CH═. In some embodiments, X¹ is optionally substituted —NH—.

In some embodiments, X² In some embodiments, —O—. In some embodiments, X² is —S—. In some embodiments, X² is —N(—)—. In some embodiments, X² is —C(—)═. In some embodiments, X² is optionally substituted —CH(—)—. In some embodiments, X² is optionally substituted —CH═. In some embodiments, X² is optionally substituted —NH—.

In some embodiments, X³ In some embodiments, —O—. In some embodiments, X³ is —S—. In some embodiments, X³ is —N(—)—. In some embodiments, X³ is —C(—)═. In some embodiments, X³ is optionally substituted —CH(—)—. In some embodiments, X³ is optionally substituted —CH═. In some embodiments, X³ is optionally substituted —NH—.

In some embodiments, X⁴ In some embodiments, —O—. In some embodiments, X⁴ is —S—. In some embodiments, X⁴ is —N(—)—. In some embodiments, X⁴ is —C(—)═. In some embodiments, X⁴ is optionally substituted —CH(—)—. In some embodiments, X⁴ is optionally substituted —CH═. In some embodiments, X⁴ is optionally substituted —NH—.

In some embodiments, Ring B is an optionally substituted 5-8, e.g., 5, 6, 7, or 8, membered monocyclic ring having 0-4 (e.g., 1-4, 1-3, 0, 1, 2, 3, 4, etc.) heteroatoms. In some embodiments, Ring B is an optionally substituted 5-8, e.g., 5, 6, 7, or 8, membered monocyclic ring having 1-4 (e.g., 1-3, 1, 2, 3, 4, etc.) heteroatoms. In some embodiments, Ring B is an optionally substituted 5-membered ring having 1-4 (e.g., 1-3, 1, 2, 3, 4, etc.) heteroatoms. In some embodiments, Ring B is an optionally substituted 6-membered ring having 1-4 (e.g., 1-3, 1, 2, 3, 4, etc.) heteroatoms. In some embodiments, Ring B is an optionally substituted 5-membered ring having 2 heteroatoms. In some embodiments, Ring B is an optionally substituted 6-membered ring having 2 heteroatoms. In some embodiments, a heteroatom is nitrogen. In some embodiments, a heteroatom is sulfur. For example, in some embodiments, Ring B is optionally substituted

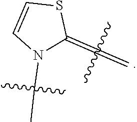 [SMILES: *N1C=CSC1=*]

In some embodiments, Ring B is

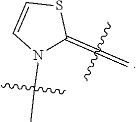 [SMILES: *N1C=CSC1=*]

### Ring C

In some embodiments, Ring C is an optionally substituted, 3-20 (e.g., 3-15, 3-10, 4-20, 5-20, 3, 4, 5, 6, 7, 8, 9, 10, 11, 12, 13, 14, 15, 16, 17, 18, 19, 20, etc.) membered, monocyclic, bicyclic or polycyclic ring having 0-6 (e.g., 1-6, 1-5, 1-4, 0, 1, 2, 3, 4, 5, 6, etc.) heteroatoms. In some embodiments, Ring C is an optionally substituted ring described herein.

In some embodiments, Ring C is an optionally substituted 5-8, e.g., 5, 6, 7, or 8, membered monocyclic non-aromatic ring having 0-4 (e.g., 1-4, 1-3, 0, 1, 2, 3, 4, etc.) heteroatoms. In some embodiments, Ring C is an optionally substituted 3-10 (e.g., 4-10, 5-10, 3, 4, 5, 6, 7, 8, 9, 10, etc.) membered monocyclic saturated ring having 0-4 (e.g., 1-4, 1-3, 0, 1, 2, 3, 4, etc.) heteroatoms. In some embodiments, Ring C is an optionally substituted 5-10 (e.g., 5-9, 5, 6, 7, 8, 9, 10, etc.) membered monocyclic saturated ring having 1 or 2 nitrogen atoms. In some embodiments, Ring C is an optionally substituted 5-membered monocyclic saturated ring having two nitrogen atoms. In some embodiments, Ring C is an optionally substituted 6-membered monocyclic saturated ring having two nitrogen atoms. In some embodiments, Ring C is an optionally substituted 7-membered monocyclic saturated ring having two nitrogen atoms. In some embodiments, Ring C is an optionally substituted 8-membered monocyclic saturated ring having two nitrogen atoms. In some embodiments, Ring C is an optionally substituted 9-membered monocyclic saturated ring having two nitrogen atoms. In some embodiments, Ring C is an optionally substituted 10-membered monocyclic saturated ring having two nitrogen atoms. In some embodiments, Ring C is bonded to the rest of the compound through the two nitrogen atoms. In some embodiments, Ring C is optionally substituted bivalent piperidyl. In some embodiments, Ring C is optionally substituted bivalent piperazinyl. In some embodiments, Ring C is optionally substituted bivalent 2,7-diazaspiro[3.5]nonyl.

In some embodiments, Ring C is of such a structure that a compound of formula E or a salt thereof is a compound of formula F or a salt thereof.

### Ring P

As described herein, Ring P is an optionally substituted 3-20 (e.g., 5, 6, 7, 8, 9, 10, 11, 12, 13, 14, 15, 16, 17, 18, 19, or 20) membered, monocyclic, bicyclic or polycyclic ring having 0-5 (e.g., 0, 1, 2, 3, 4 or 5) heteroatoms. In some embodiments, Ring P is monocyclic. In some embodiments, Ring P is bicyclic. In some embodiments, Ring P is polycyclic. In some embodiments, Ring P is saturated. In some embodiments, Ring P is partially unsaturated. In some embodiments, Ring P is aromatic. In some embodiments, Ring P is 3-membered. In some embodiments, it is 4-membered. In some embodiments, it is 5-membered. In some embodiments, it is 6-membered. In some embodiments, it is 7-membered. In some embodiments, it is 8-membered. In some embodiments, it is 9-membered. In some embodiments, it is 10-membered. In some embodiments, it is 11-membered. In some embodiments, it is 12-membered. In some embodiments, it is 13-membered. In some embodiments, it is 14-membered. In some embodiments, it is 15-membered. In some embodiments, it is 16-membered. In some embodiments, it is 17-membered. In some embodiments, it is 18-membered. In some embodiments, it is 19-membered. In some embodiments, it is 20-membered. In some embodiments, a monocyclic ring unit in Ring P is an optionally substituted 5-6 membered aromatic ring having 0-4 (e.g., 0, 1, 2, 3, or 4) heteroatoms and comprises Z and Y as ring atoms. In some embodiments, such an aromatic ring is 5-membered. In some embodiments, it is 6-membered. In some embodiments, it is substituted. In some embodiments, it is not substituted. In some embodiments, there is one heteroatom in such an aromatic ring; in some embodiments, there are two; in some embodiments, there are three; in some embodiments, there are four; in some embodiments, at least one heteroatom is nitrogen. In some embodiments, Ring P is an optionally substituted phenyl ring. In some embodiments, Ring P is an optionally substituted pyrrole ring. In some embodiments, Ring P is an optionally substituted indole ring.

In some embodiments, each monocyclic ring unit in Ring P is independently an optionally substituted, 3-10, 3-8, 3-7, 3-6, 4-10, 5-10, 5-9, 5-8, 5-7, 5-6, 3, 4, 5, 6, 7, 8, 9, or 10 membered, saturated, partially unsaturated or aromatic ring having 0-4 (e.g., 0, 1, 2, 3 or 4) heteroatoms. In some embodiments, each monocyclic ring unit is independently 3-7 membered. In some embodiments, a monocyclic ring unit is 3-membered. In some embodiments, a monocyclic ring unit is 4-membered. In some embodiments, a monocyclic ring unit is 5-membered. In some embodiments, a monocyclic ring unit is 6-membered. In some embodiments, a monocyclic ring unit is 7-membered. In some embodiments, a monocyclic ring unit is 8-membered. In some embodiments, a monocyclic ring unit is 9-membered. In some embodiments, a monocyclic ring unit is 10-membered. In some embodiments, as described herein, each heteroatom is independently selected nitrogen, oxygen and sulfur. In some embodiments, a monocyclic ring unit is saturated. In some embodiments, a monocyclic ring unit is partially unsaturated. In some embodiments, a monocyclic ring unit is aromatic. In some embodiments, a monocyclic ring unit is heteroaromatic. In some embodiments, there is one ring atom of a monocyclic ring unit that is a heteroatom. In some embodiments, there are two or more ring atoms of a monocyclic ring unit each of which is independently a heteroatom. In some embodiments, there are three or more ring atoms of a monocyclic ring unit each of which is independently a heteroatom. In some embodiments, there are four or more ring atoms of a monocyclic ring unit each of which is independently a heteroatom.

### R^{s}

In some embodiments, R^{s} is R″ as described herein. In some embodiments, R^{s} is R′ as described herein. In some embodiments, R^{s} is R as described herein.

In some embodiments, R^{s} is —H. In some embodiments, R′ is not —H.

In some embodiments, R^{s} is halogen. In some embodiments, R′ is —F. In some embodiments, R^{s} is —Cl. In some embodiments, R^{s} is —Br. In some embodiments, R^{s} is —I. In some embodiments, R^{s} is —CN. In some embodiments, R^{s} is oxo. In some embodiments, R^{s} is —NO₂. In some embodiments, R′ is optionally substituted acyl. In some embodiments, R^{s} is optionally substituted acylamino. In some embodiments, R^{s} is hydroxy. In some embodiments, R^{s} is optionally substituted amino acid. In some embodiments, R^{s} is optionally substituted amine. In some embodiments, R^{s} is optionally substituted amide. In some embodiments, R^{s} is optionally substituted carbamate. In some embodiments, R^{s} is optionally substituted ester. In some embodiments, R^{s} is optionally substituted ether. In some embodiments, R^{s} is optionally substituted carboxylic acid. In some embodiments, R^{s} is optionally substituted thio. In some embodiments, R^{s} is optionally substituted thioalkyl. In some embodiments, R^{s} is optionally substituted thioester. In some embodiments, R^{s} is optionally substituted thioether. In some embodiments, R^{s} is optionally substituted sulfate. In some embodiments, R is optionally substituted sulfonamide. In some embodiments, R^{s} is optionally substituted sulfoxide. In some embodiments, R^{s} is optionally substituted sulfonate. In some embodiments, R^{s} is optionally substituted sulfone. In some embodiments, R^{s} is optionally substituted alkylsulfonyl. In some embodiments, R^{s} is optionally substituted arylsulfonyl. In some embodiments, when there are two or more R^{s}, they can be the same or different and each is independently as described herein.

In some embodiments, there is one occurrence of R^{s} bonded to a moiety, e.g., a ring. In some embodiments, two or more occurrences of R^{s} are bonded to a moiety, e.g., a ring, wherein each occurrence of R^{s} is independently as described herein. In some embodiments, an occurrence of R^{s} is halogen. In some embodiments, an occurrence of R^{s} is —F. In some embodiments, an occurrence of R^{s} is —Cl. In some embodiments, an occurrence of R^{s} is —N(R′)₂. In some embodiments, each occurrence of R^{s} is independently —H, —N(R)₂, —OR, —CN, and optionally substituted C₁₋₆ aliphatic. In some embodiments, an occurrence of R^{s} is —N(R)₂, wherein each R is independently —H or optionally substituted C₁₋₂₀ aliphatic. In some embodiments, an occurrence of R^{s} is —N(R)₂, wherein each R is independently —H or optionally substituted C₁₋₁₀ aliphatic. In some embodiments, an occurrence of R^{s} is —NHR, wherein each R is independently —H or optionally substituted C₁₋₂₀ aliphatic. In some embodiments, an occurrence of R^{s} is —NHR, wherein each R is independently —H or optionally substituted C₁₋₁₀ aliphatic. In some embodiments, each R^{s} is independently —H, —NH₂, —CN, —CH₃. In some embodiments, an occurrence of R^{s} is optionally substituted C₁₋₆ aliphatic. In some embodiments, an occurrence of R^{s} is optionally substituted methyl. In some embodiments, an occurrence of R^{s} is methyl optionally substituted with 1-3 fluoro. In some embodiments, an occurrence of R^{s} is —CF₃. In some embodiments, an occurrence of R⁵ is optionally substituted

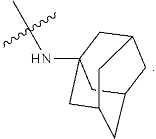 [SMILES: *NC12CC3CC(CC(C3)C1)C2]

In some embodiments, one occurrence of R^{s} is optionally substituted

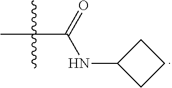 [SMILES: *C(=O)NC1CCC1]

In some embodiments, an occurrence of R^{s} is para relative to the position at which Ring A is attached to the rest of the compound. In some embodiments, each R^{s} is independently halogen, or optionally substituted C₁₋₂₀ aliphatic or C₁₋₂₀ heteroaliphatic having 1-10 heteroatoms. In some embodiments, an occurrence of R^{s} is —OCH₃. In some embodiments, an occurrence of R^{s} is —OCF₃. In some embodiments, an occurrence of R^{s} is —S(O)₂—R′. In some embodiments, an occurrence of R^{s} is —S(O)₂—R. In some embodiments, an occurrence of R^{s} is —S(O)₂—CH₃.

### R^{s6}

In some embodiments, R^{s6} is R^{s} as described herein. In some embodiments, R^{s6} is R″ as described herein. In some embodiments, R^{s6} is R′ as described herein. In some embodiments, R^{s6} is R as described herein.

In some embodiments, R^{s6} is —H. In some embodiments, R^{s6} is not —H. In some embodiments, R^{s6} is halogen. In some embodiments, R^{s6} is —F. In some embodiments, R^{s6} is —Cl. In some embodiments, R^{s6} is —Br. In some embodiments, R^{s6} is —I. In some embodiments, R^{s6} is —CN. In some embodiments, each R^{s6} is independently halogen or —CN. In some embodiments, each R^{s6} is independently —F or —Cl.

### R^{s7}

In some embodiments, R^{s7} is R^{s} as described herein. In some embodiments, R^{s7} is R″ as described herein. In some embodiments, R^{s7} is R′ as described herein. In some embodiments, R^{s7} is R as described herein.

In some embodiments, R^{s7} is —H. In some embodiments, R^{s7} is not —H. In some embodiments, R^{s7} is halogen. In some embodiments, R^{s7} is —F. In some embodiments, R^{s7} is —Cl. In some embodiments, R^{s7} is —Br. In some embodiments, R^{s7} is —I. In some embodiments, R^{s7} is —CN. In some embodiments, each R^{s7} is independently halogen or —CN. In some embodiments, each R^{s7} is independently —F or —Cl.

### R^{8a}

In some embodiments, R^{8a} is R^{s} as described herein. In some embodiments, R^{8a} is optionally substituted C₁₋₆ aliphatic. In some embodiments, R^{8a} is optionally substituted methyl.

In some embodiments, R^{8a} is -L-R′ wherein each variable is independently as described herein. In some embodiments, R′ is optionally substituted 6-10 membered aryl. In some embodiments, R′ is optionally substituted phenyl. In some embodiments, R^{8a} is -L-R wherein each variable is independently as described herein. In some embodiments, L is optionally substituted —CH₂—. In some embodiments, L is —CH₂—. In some embodiments, R is not —H. In some embodiments, R is optionally substituted phenyl. For example, in some embodiments, R is 2-chlorophenyl.

In some embodiments, R^{Ra} is

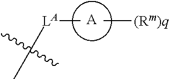

wherein each variable is independently as described herein. In some embodiments, L^{A} is optionally substituted —CH₂—. In some embodiments, L^{A} is —CH₂—. In some embodiments, Ring A is optionally substituted phenylene. In some embodiments, Ring A is optionally substituted 1, 2-phenylene. In some embodiments, q is 0. In some embodiments, q is 1-10. In some embodiments, q is 1. In some embodiments, each R^{m} is independently R^{s} as described herein. In some embodiments, R^{m} is halogen. In some embodiments, each R^{m} is independently halogen. In some embodiments, R^{m} is —F. In some embodiments, R^{m} is —Cl. In some embodiments, R^{m} is —Br. In some embodiments, R^{m} is —I. In some embodiments, each R^{m} that is halogen is independently —F or —Cl. In some embodiments, each substituent on Ring A, if any, is independently halogen. In some embodiments, it is independently —F or —Cl. In some embodiments, it is —Cl.

### R^{m}

In some embodiments, R^{m} is R^{s} as described herein. In some embodiments, R^{m} is R″ as described herein. In some embodiments, R^{m} is R′ as described herein. In some embodiments, R^{m} is R as described herein.

In some embodiments, R^{m} is halogen. In some embodiments, R^{m} is —F. In some embodiments, R′ is —Cl. In some embodiments, R^{m} is —Br. In some embodiments, R^{m} is —I. In some embodiments, R^{m} is —CN. In some embodiments, R^{m} is oxo. In some embodiments, R^{m} is —NO₂. In some embodiments, R^{m} is an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl.

### L^{R}

In some embodiments, L^{R}

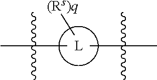

wherein each variable is independently as described herein.
In some embodiments, L^{R} is

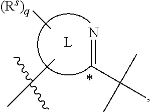

wherein “*” indicates the atom bonded to R⁴, and Ring L is as described herein. In some embodiments, LR is

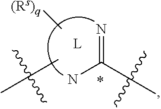

wherein “*” indicates the atom bonded to R⁴, and Ring L is as described herein. For example, in some embodiments, L^{R} is optionally substituted

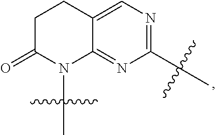 [SMILES: *c1ncc2c(n1)N(*)C(=O)CC2]

wherein the nitrogen atom is boned to L^{b}.

In some embodiments, L^{R} is an optionally substituted bicyclic or tricyclic ring, wherein a monocyclic ring is an optionally substituted 6-membered heteroaryl ring comprising ═N—C*(—)═N—, wherein “*” indicates the atom bonded to R⁴. In some embodiments, L^{b} is bonded to a different monocyclic ring. In some embodiments, L^{R} is optionally substituted

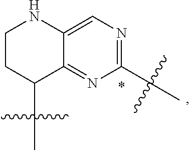 [SMILES: *c1ncc2c(n1)C(*)CCN2]

wherein “*” indicates the atom bonded to R⁴. In some embodiments, L^{R} is optionally substituted

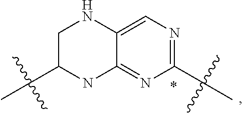 [SMILES: *c1ncc2c(n1)NC(*)CN2]

wherein “*” indicates the atom bonded to R⁴. In some embodiments, L^{R} is optionally substituted

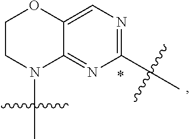 [SMILES: *c1ncc2c(n1)N(*)CCO2]

wherein “*” indicates the atom bonded to R⁴. In some embodiments, L^{R} is optionally substituted

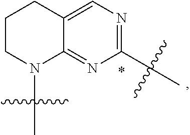 [SMILES: *c1ncc2c(n1)N(*)CCC2]

wherein “*” indicates the atom bonded to R⁴. In some embodiments, L^{R} is optionally substituted

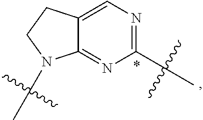 [SMILES: *c1ncc2c(n1)N(*)CC2]

wherein “*” indicates the atom bonded to R⁴. In some embodiments, L^{R} is optionally substituted

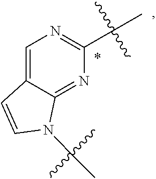 [SMILES: *c1ncc2ccn(*)c2n1]

wherein “*” indicates the atom bonded to R⁴. In some embodiments, L^{R} is optionally substituted

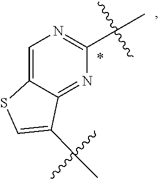 [SMILES: *c1ncc2scc(*)c2n1]

wherein “*” indicates the atom bonded to R⁴. In some embodiments, L^{R} is optionally substituted

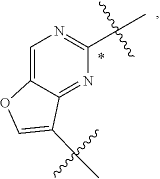 [SMILES: *c1ncc2occ(*)c2n1]

wherein “*” indicates the atom bonded to R⁴. In some embodiments, L^{R} is optionally substituted

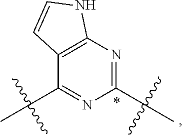 [SMILES: *c1nc(*)c2cc[nH]c2n1]

wherein
indicates the atom bonded to R⁴. In some embodiments, L^{R} is optionally substituted

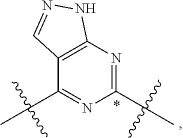 [SMILES: *c1nc(*)c2cn[nH]c2n1]

wherein “*” indicates the atom bonded to R⁴. In some embodiments, L^{R} is optionally substituted

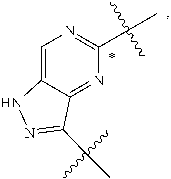 [SMILES: *c1ncc2[nH]nc(*)c2n1]

wherein “*” indicates the atom bonded to R⁴. In some embodiments, L^{R} is optionally substituted

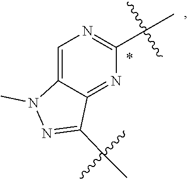 [SMILES: *c1ncc2c(n1)c(*)nn2C]

wherein “*” indicates the atom bonded to R⁴. In some embodiments, L^{R} is optionally substituted

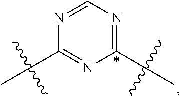 [SMILES: *c1ncnc(*)n1]

“*” indicates the atom bonded to R⁴. In some embodiments, L^{R} is optionally substituted

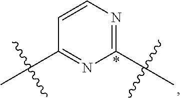 [SMILES: *c1ccnc(*)n1]

wherein “*” indicates the atom bonded to R⁴. In some embodiments, L^{R} is optionally substituted

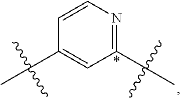 [SMILES: *c1ccnc(*)c1]

wherein “*” indicates the atom bonded to R⁴. In some embodiments, L^{R} is optionally substituted

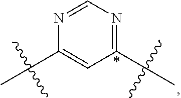 [SMILES: *c1cc(*)ncn1]

wherein “*” indicates the atom bonded to R⁴. In some embodiments, L^{R} is optionally substituted

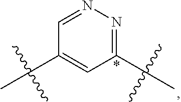 [SMILES: *c1cnnc(*)c1]

wherein “*” indicates the atom bonded to R⁴. In some embodiments, L^{R} is optionally substituted

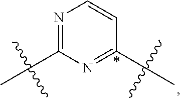 [SMILES: *c1ccnc(*)n1]

wherein “*” indicates the atom bonded to R⁴. In some embodiments, L^{R} is optionally substituted

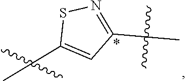 [SMILES: *c1cc(*)sn1]

wherein “*” indicates the atom bonded to R⁴. In some embodiments, L^{R} is optionally substituted

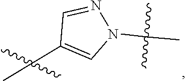 [SMILES: *c1cnn(*)c1]

wherein “*” indicates the atom bonded to R⁴. In some embodiments, a ring may exist is a tautomeric form (e.g., —C(OH)═ and —C(O)—).

In some embodiments, L^{R} is -Cy- as described herein.

In some embodiments, L^{R} is

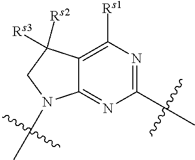

wherein each of R^{s1}, R^{s2} and R^{s3} is independently R^{s} as described herein. In some embodiments, L^{R} is

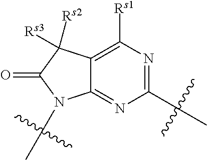

wherein each of R^{s1}, R^{s2} and R^{s3} is independently R^{s} as described herein. In some embodiments, the nitrogen atom is bonded to L^{b}. In some embodiments, L^{R} is

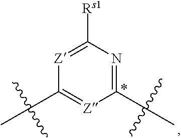

wherein each variable is independently as described herein, and “*” indicates the atom bonded to R⁴. In some embodiments, L^{R} is

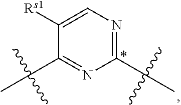

wherein each variable is independently as described herein, and “*” indicates the atom bonded to R⁴. In some embodiments, L^{R} is

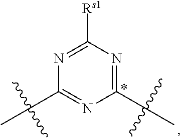

wherein each variable is independently as described herein, and “*” indicates the atom bonded to R⁴.

In some embodiments, Z′ is —C(R^{s4}) wherein R^{s4} is R^{s} as described herein. In some embodiments, Z′ is —C(R^{s4}) wherein R^{s4} is R′ as described herein. In some embodiments, Z′ is —C(R^{s4}) wherein R^{s4} is R as described herein. In some embodiments, R^{s4} is an optionally substituted group selected from C₁₋₁₀ (e.g., C₁₋₉, C₁₋₄, C₁₋₆, C₂₋₁₀, C₃₋₁₀, C₄₋₁₀, C₁, C₂, C₃, C₄, C₅, C₆, C₇, C₈, C₉, C₁₀, etc.) aliphatic, C₁₋₁₀ (e.g., C₁₋₉, C₁₋₉, C₁₋₆, C₂₋₁₀, C₃₋₁₀, C₄₋₁₀, C₁, C₂, C₃, C₄, C₅, C₆, C₇, C₈, C₉, C₁₀, etc.) heteroaliphatic having 1-5 (e.g., 1-4, 1, 2, 3, 4, 5, etc.) heteroatoms, 6-10 (e.g., 6, 7, 8, 9, 10, etc.) membered aryl, 5-10 (e.g., 5, 6, 7, 8, 9, 10, etc.) membered heteroaryl having 1-4 (e.g., 1-3, 1, 2, 3, 4, etc.) heteroatoms, and 3-10 (e.g., 4-10, 5-10, 5-7, 5-6, 3, 4, 5, 6, 7, 8, 9, 10, etc.) membered heterocyclyl having 1-5 (e.g., 1-4, 2-5, 1, 2, 3, 4, 5, etc.) heteroatoms. In some embodiments, R^{s4} is —H. In some embodiments, R^{s4} is -halogen. In some embodiments, R^{s4} is —F. In some embodiments, R^{s4} is —Cl. In some embodiments, R⁴ is —Br. In some embodiments, R⁵ is —I. In some embodiments, R^{s4} is —CN. In some embodiments, R⁴ is —N(R′)₂ wherein each R′ is independently as described herein. In some embodiments, R^{s4} is —N(R′)₂ wherein each R′ is independently —H or optionally substituted C₁₋₆ aliphatic. In some embodiments, R^{s4} is —N(R′)₂ wherein each R′ is independently optionally substituted C₁₋₆ aliphatic. In some embodiments, R⁴ is optionally substituted —NH₂. In some embodiments, R^{s4} is —NH₂. In some embodiments, R^{s4} is optionally substituted C₁₋₁₀ (e.g., C₁₋₉, C₁₋₈, C₁₋₆, C₂₋₁₀, C₃₋₁₀, C₄₋₁₀, C₁, C₂, C₃, C₄, C₅, C₆, C₇, C₈, C₉, C₁₀, etc.) aliphatic. In some embodiments, R^{s4} is optionally substituted C₁₋₁₀ (e.g., C₁₋₉, C₁₋₈, C₁₋₆, C₂₋₁₀, C₃₋₁₀, C₄₋₁₀, C₁, C₂, C₃, C₄, C₅, C₆, C₇, C₈, C₉, C₁₀, etc.) heteroaliphatic having 1-5 (e.g., 1-4, 2-5, 1, 2, 3, 4, 5, etc.) heteroatoms. In some embodiments, R^{s4} is optionally substituted 3-10 (e.g., 3-9, 4-10, 5-10, 3, 4, 5, 6, 7, 8, 9, 10, etc.) membered heterocyclyl having 1-5 (e.g., 1-4, 2-5, 1, 2, 3, 4, 5, etc.) heteroatoms. In some embodiments, R^{s4} is

optionally substituted

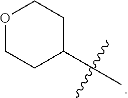 [SMILES: *C1CCOCC1]

In some embodiments, Z″ is —C(R^{s5}) wherein R^{s5} is R^{s} as described herein. In some embodiments, Z″ is —C(R^{s5}) wherein R^{s5} is R′ as described herein. In some embodiments, Z″ is —C(R^{s5}) wherein R^{s5} is R as described herein. In some embodiments, R^{s5} is —H. In some embodiments, R^{s5} is -halogen. In some embodiments, R^{s5} is —F. In some embodiments, R^{s5} is —Cl. In some embodiments, R^{s5} is —Br. In some embodiments, R^{s5} is —I. In some embodiments, R^{s5} is —CN. In some embodiments, R^{s5} is —N(R′)₂ wherein each R′ is independently as described herein. In some embodiments, R^{s5} is —N(R′)₂ wherein each R′ is independently —H or optionally substituted C₁₋₆ aliphatic. In some embodiments, R″ is —N(R′)₂ wherein each R′ is independently optionally substituted C₁₋₆ aliphatic. In some embodiments, R^{s5} is optionally substituted —NH₂. In some embodiments, R^{s5} is —NH₂. In some embodiments, R^{s5} is optionally substituted C₁₋₁₀ (e.g., C₁₋₉, C₁₋₈, C₁₋₆, C₂₋₁₀, C₃₋₁₀, C₄₋₁₀, C₁, C₂, C₃, C₄, C₅, C₆, C₇, C₈, C₉, C₁₀, etc.) aliphatic. In some embodiments, R^{s5} is optionally substituted C₁₋₁₀ (e.g., C₁₋₉, C₁₋₈, C₁₋₆, C₂₋₁₀, C₃₋₁₀, C₄₋₁₀, C₁, C₂, C₃, C₄, C₅, C₆, C₇, C₈, C₉, C₁₀, etc.) heteroaliphatic having 1-5 (e.g., 1-4, 2-5, 1, 2, 3, 4, 5, etc.) heteroatoms. In some embodiments, R^{s} is optionally substituted 3-10 (e.g., 3-9, 4-10, 5-10, 3, 4, 5, 6, 7, 8, 9, 10, etc.) membered heterocyclyl having 1-5 (e.g., 1-4, 2-5, 1, 2, 3, 4, 5, etc.) heteroatoms. In some embodiments, R^{s5} is —OR wherein R is as described herein. In some embodiments, R^{s5} is —OR wherein R is optionally substituted C₁₋₆ aliphatic. In some embodiments, R^{s5} is —OH. In some embodiments, R^{s5} is —OCH₃.

In some embodiments, R^{s1} is R^{s} as described herein. In some embodiments, R^{s1} is R′ as described herein. In some embodiments, R^{s1} is R as described herein. In some embodiments, R^{s1} is —N(R′)₂ wherein each R′ is independent as described herein. In some embodiments, R^{s1} is —N(R′)₂, wherein each R′ is independently —H or optionally substituted C₁₋₆ aliphatic. In some embodiments, In some embodiments, R^{s1} is —NHR′ wherein R′ is optionally substituted C₁₋₆ aliphatic. In some embodiments, R^{s1} is —NHR′ wherein R′ is —H or optionally substituted C₁₋₆ alkyl. In some embodiments, R^{s1} is optionally substituted —NH₂. In some embodiments, R^{s1} is —NH₂. In some embodiments, R^{s1} is —NHCH₃. In some embodiments, R^{s1} is —NHCD₃. In some embodiments, R^{s1} is —NHCH₂CH₃. In some embodiments, R^{s1} is —NHCH₂CH₂CH₃. In some embodiments, R^{s1} is —NHCH(CH₃)₂. In some embodiments, R^{s1} is —N(CH₃)₂. In some embodiments, R^{s1} is

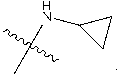 [SMILES: *NC1CC1]

In some embodiments, R^{s1} is

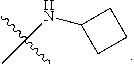 [SMILES: *NC1CCC1]

In some embodiments, R^{s1} is —NHCH₂CF₃. In some embodiments, R^{s1} is —NHCH₂CHF₂. In some embodiments, R^{s1} is —NHCH₂C≡CH. In some embodiments, R^{s1} is —NHCH₂CH═CH₂. In some embodiments, R^{s1} is optionally substituted

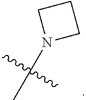 [SMILES: *N1CCC1]

In some embodiments, R^{s1} is

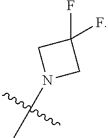 [SMILES: *N1CC(F)(F)C1]

In some embodiments, R^{s1} is

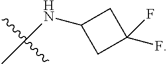 [SMILES: *NC1CC(F)(F)C1]

In some embodiments, R^{s1} is —OR′ wherein R′ is as described herein. In some embodiments, R^{s1} is —OR wherein R is as described herein. In some embodiments, R^{s1} is —OR wherein R is optionally substituted C₁₋₆ aliphatic. In some embodiments, R^{s1} is —OH. In some embodiments, when R^{s1} is —OH a ring or compound may exist in a tautomeric form. In some embodiments, —C(OH)═ may exist as —C(O)—. In some embodiments, —C(OH)═N— may exist as —C(O)—NH—. In some embodiments, R^{s1} is —OCH₃. In some embodiments, R^{s1} is halogen. In some embodiments, R^{s1} is —F. In some embodiments, R^{s1} is —Cl. In some embodiments, R^{s1} is —Br. In some embodiments, R^{s1} is —I. In some embodiments, R^{s1} is —H. In some embodiments, R^{s1} is —CF₃. In some embodiments, R^{s1} is —CHF₂.

In some embodiments, R^{s4} is optionally substituted

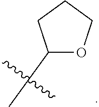 [SMILES: *C1CCCO1]

In some embodiments, R^{s4} is

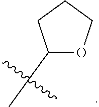 [SMILES: *C1CCCO1]

In some embodiments, R^{s4} is optionally substituted

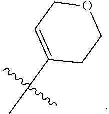 [SMILES: *C1=CCOCC1]

In some embodiments, R^{s4} is

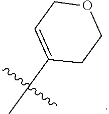 [SMILES: *C1=CCOCC1]

In some embodiments, R^{s4} is optionally substituted

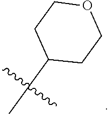 [SMILES: *C1CCOCC1]

In some embodiments, R^{s4} is

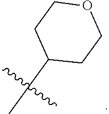 [SMILES: *C1CCOCC1]

In some embodiments, R^{s4} is —N(R′)₂. In some embodiments, R^{s4} is —NH₂. In some embodiments, R^{s4} is —OR′. In some embodiments, R^{s4} is —O(CH(CH₃)₂. In some embodiments, R^{s4} is optionally substituted C₁₋₆ aliphatic. In some embodiments, R^{s4} is C₁₋₆ aliphatic optionally substituted with —OR [symbol] , wherein R [symbol] is —H or C₁₋₆ aliphatic. In some embodiments, R^{s4} is C(CH₃)₂OH.

In some embodiments, R^{s2} is R^{s} as described herein. In some embodiments, R^{s2} is R′ as described herein. In some embodiments, R^{s} is R as described herein. In some embodiments, R^{s2} is —H. In some embodiments, R^{s2} is optionally substituted C₁₋₆ aliphatic. In some embodiments, R^{s2} is optionally substituted C₁₋₆ alkyl.

In some embodiments, R^{s3} is R^{s} as described herein. In some embodiments, R^{s3} is R′ as described herein. In some embodiments, R^{s3} is R as described herein. In some embodiments, R³ is —H. In some embodiments, R^{s3} is optionally substituted C₁₋₆ aliphatic. In some embodiments, R^{s3} is optionally substituted C₁₋₆ alkyl.

In some embodiments, each of R^{s2} and R^{s3} is independently R as described herein. In some embodiments, both are —H. In some embodiments, each is independently —H or C₁₋₆ aliphatic. In some embodiments, each is independently optionally substituted C₁₋₆ aliphatic. In some embodiments, each is independently optionally substituted C₁₋₆ alkyl. In some embodiments, R^{s2} and R^{s3} are the same. In some embodiments, R^{s2} and R^{s3} are different. In some embodiments, both R^{s2} and R^{s3} are methyl. In some embodiments, R^{s2} is —H, and R^{s3} is methyl.

In some embodiments, R^{s2} and R^{s3} are taken together with the carbon atom to which they are attached to form an optionally substituted 3-10 (e.g., 3-9, 3-8, 4-10, 5-10, 5-9, 5-8, 5-7, 3, 4, 5, 6, 7, 8, 9, 10, etc.) membered saturated or partially unsaturated ring having 0-4 (e.g., 1-4, 0, 1, 2, 3, 4, etc.) heteroatoms. In some embodiments, R^{s2} and R^{s3} are taken together with the carbon atom to which they are attached to form an optionally substituted 3-6 membered saturated or partially unsaturated carbocyclyl ring. In some embodiments, R^{s2} and R^{s3} are taken together with the carbon atom to which they are attached to form an optionally substituted 3-6 membered saturated or partially unsaturated ring having 1-4 heteroatoms. In some embodiments, a heteroatom is nitrogen. In some embodiments, a heteroatom is oxygen. In some embodiments, a heteroatom is sulfur. In some embodiments, a formed ring is saturated. In some embodiments, a formed ring is partially unsaturated. In some embodiments, a formed ring 3-membered. In some embodiments, a formed ring is 4-membered. In some embodiments, a formed ring is 5-membered. In some embodiments, a formed ring is 6-membered. In some embodiments, R^{s2} and R^{s3} are taken together with the carbon atom to which they are attached to form an optionally substituted cyclopropyl ring. In some embodiments, R^{s2} and R^{s3} are taken together with the carbon atom to which they are attached to form a cyclopropyl ring. In some embodiments, R^{s2} and R^{s3} are taken together with the carbon atom to which they are attached to form a cyclobutyl ring. In some embodiments, R^{s2} and R^{s3} are taken together with the carbon atom to which they are attached to form a cyclopentyl ring. In some embodiments, R^{s2} and R^{s3} are taken together with the carbon atom to which they are attached to form a cyclohexyl ring. In some embodiments, R^{s2} and R^{s3} are taken together with the carbon atom to which they are attached to form an optionally substituted 3-6 membered saturated ring having an oxygen atom. In some embodiments, R^{s2} and R^{s3} are taken together with the carbon atom to which they are attached to form optionally substituted

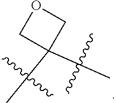 [SMILES: *C1(*)COC1]

In some embodiments, R^{s2} and R^{s3} are taken together with the carbon atom to which they are attached to form

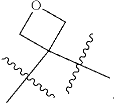 [SMILES: *C1(*)COC1]

In some embodiments, R^{s2} and R^{s3} are taken together with the carbon atom to which they are attached to form optionally substituted

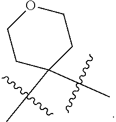 [SMILES: *C1(*)CCOCC1]

In some embodiments, R^{s2} and R^{s3} are taken together with the carbon atom to which they are attached to form

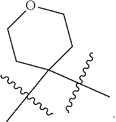 [SMILES: *C1(*)CCOCC1]

In some embodiments, L^{R} is L as described herein. In some embodiments, L^{R} is —C≡C—. In some embodiments, L^{R} is optionally substituted —CH═CH—. In some embodiments, L^{R} is —C(O)—. In some embodiments, L^{R} is —C(S)—. In some embodiments, L^{R} is —C(NR″)— wherein R″ is as described herein. In some embodiments, L^{R} is a covalent bond. In some embodiments, L^{R} is L as described herein. In some embodiments, L^{R} is optionally substituted C₁₆ (e.g., C₁₋₄, C₁, C₂, C₃, C₄, C₅, C₆, etc.) alkylene. In some embodiments, L^{R} is optionally substituted C₂₋₆(e.g., C₂, C₃, C₄, C₅, C₆, etc.) alkenylene. In some embodiments, L^{R} is optionally substituted C₂₋₆(e.g., C₂, C₃, C₄, C₅, C₆, etc.) alkynylene. In some embodiments, L^{R} is -Cy- as described herein. In some embodiments, L^{R} is optionally substituted C₃₋₁₀ (e.g., C₄₋₁₀, C₅₋₁₀, C₃, C₄, C₅, C₆, C₇, C₈, C₉, C₁₀, etc.) cycloalkylene. In some embodiments, L^{R} is optionally substituted C₆₋₁₄ (e.g., C₆, C₁₀, etc.) arylene. In some embodiments, L^{R} is optionally substituted C₇₋₁₅ aralkylene. In some embodiments, L^{R} is optionally substituted 5-14 (e.g., 5-10, 5-9, 5, 6, 9, 10, 14, etc.) membered heteroarylene having 1-5 (e.g., 1-2, 1, 2, 3, 4, 5, etc.) heteroatoms. In some embodiments, L^{R} is optionally substituted 3-14 (e.g., 3-20, 5-10, 5-10, 5-6, 3, 4, 5, 6, 7, 8, 9, 10, 11, 12, 13, 14, etc.) membered, or heterocyclylene having 1-5 (e.g., 1-3, 1, 2, 3, 4, 5, etc.) heteroatoms.

### Ring L

In some embodiments, Ring L is an optionally substituted, 3-20 membered, monocyclic, bicyclic or polycyclic ring having 0-10 heteroatoms as described herein. In some embodiments, Ring L is a ring as described herein, e.g., in section Ring A. In some embodiments, Ring L is or comprises an optionally substituted monocyclic aromatic ring unit having 0-4 (e.g., 0-3, 2-4, 0, 1, 2, 3, 4, etc.) heteroatoms. In some embodiments, Ring L is or comprises an optionally substituted monocyclic aromatic unit having 1-4 heteroatoms. In some embodiments, Ring L comprises N═C*(—)—, wherein “*” indicates the atom bonded to R⁴. In some embodiments, Ring L comprises N═C*(—)—N═, wherein “*” indicates the atom bonded to R⁴. In some embodiments, Ring L is

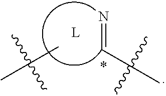

In some embodiments, Ring L is

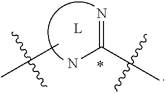

Ring L is or comprises an optionally substituted bicyclic aromatic unit having 0-6 heteroatoms.

In some embodiments, Ring L is an optionally substituted, 5-20 (e.g., 5-15, 5-10, 5-9, 5, 6, 7, 8, 9, 10, 11, 12, 13, 14, 15, 16, 17, 18, 19, 20, etc.) membered, monocyclic, bicyclic or polycyclic ring having 1-10 (e.g., 2-10, 3-10, 2-5, 2-4, 1, 2, 3, 4, 5, 6, 7, 8, 9, 10, etc.) heteroatoms. In some embodiments, Ring L is monocyclic. In some embodiments, Ring L is bicyclic. In some embodiments, Ring L is polycyclic. In some embodiments, Ring L is or comprises an optionally substituted 6-membered monocyclic aromatic ring unit having 0-4 (e.g., 1-4, 0, 1, 2, 3, 4, etc.) heteroatoms. In some embodiments, Ring L is or comprises an optionally substituted monocyclic non-aromatic ring unit having 0-4 (e.g., 1-4, 0, 1, 2, 3, 4, etc.) heteroatoms. In some embodiments, the monocyclic ring unit bonded to R⁴ is an optionally substituted 5-6 membered heteroaryl ring having 1-5 (e.g., 1-4, 2-5, 2-4, 1, 2, 3, 4, 5, etc.) heteroatoms. In some embodiments, the monocyclic ring unit bonded to R⁴ is an optionally substituted 6 membered heteroaryl ring having 1-5 (e.g., 1-4, 2-5, 2-4, 1, 2, 3, 4, 5, etc.) heteroatoms. In some embodiments, the monocyclic ring unit bonded to R⁴ is an optionally substituted 6 membered heteroaryl ring having 2-5 (e.g., 2-4, 2, 3, 4, 5, etc.) heteroatoms. In some embodiments, it has at least one nitrogen atom. In some embodiments, it has at least two nitrogen atoms. In some embodiments, Ring L is bicyclic or polycyclic. In some embodiments, Ring L comprises an optionally substituted partially unsaturated monocyclic ring unit. In some embodiments, Ring L comprises a 3-10 (e.g., 5-10, 5-10, 5-6, 3, 4, 5, 6, 7, 8, 9, 10, etc.) membered partially unsaturated ring having 0-6 (e.g., 1-6, 1-3, 0, 1, 2, 3, 4, 5, 6, etc.) heteroatoms. In some embodiments, Ring L comprises a 5-6 membered partially unsaturated ring having 1-3 (e.g., 1-2, 1, 2, 3, etc.) heteroatoms. In some embodiments, unsaturation is due to fusion to an aromatic ring unit, e.g., the monocyclic ring unit bonded to R⁴. In some embodiments, in addition to a monocyclic ring unit bonded to R⁴, Ring L comprises a monocyclic ring unit which is a monocyclic aromatic ring unit. In some embodiments, such a monocyclic aromatic unit is an optionally substituted 5-6 membered aromatic ring having 0-4 (e.g., 1-4, 0, 1, 2, 3, 4, etc.) heteroatoms. In some embodiments, such a monocyclic aromatic unit is an optionally substituted 5-membered heteroaryl having 1-4 (e.g., 1-4, 1-3, 1-2, 1, 2, 3, 4, etc.). In some embodiments, at least one heteroatom is nitrogen. In some embodiments, each heteroatom is nitrogen.

In some embodiments, Ring L is or comprises an optionally substituted bicyclic aromatic unit having 0-6 heteroatoms. In some embodiments, Ring L is or comprises an optionally substituted 9-membered bicyclic aromatic unit having 0-6 heteroatoms.

In some embodiments, Ring L is optionally substituted

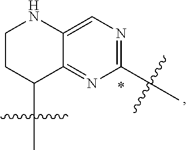 [SMILES: *c1ncc2c(n1)C(*)CCN2]

wherein “*” indicates the atom bonded to R⁴. In some embodiments, Ring L is optionally substituted

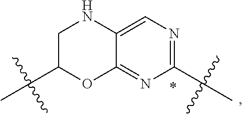 [SMILES: *c1ncc2c(n1)OC(*)CN2]

wherein “*” indicates the atom bonded to R⁴. In some embodiments, Ring L is optionally substituted

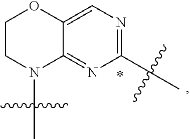 [SMILES: *c1ncc2c(n1)N(*)CCO2]

wherein “*” indicates the atom bonded to R⁴. In some embodiments, Ring L is optionally substituted

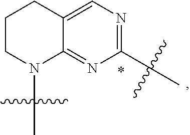 [SMILES: *c1ncc2c(n1)N(*)CCC2]

wherein “*” indicates the atom bonded to R⁴. In some embodiments, Ring L is optionally substituted

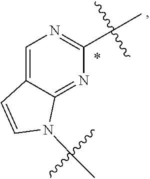 [SMILES: *c1ncc2ccn(*)c2n1]

wherein “*” indicates the atom bonded to R⁴. In some embodiments, Ring L is optionally substituted

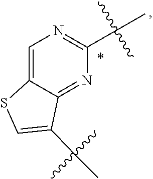 [SMILES: *c1ncc2scc(*)c2n1]

wherein “*” indicates the atom bonded to R⁴. In some embodiments, Ring L is optionally substituted

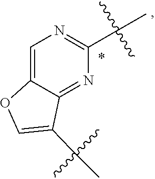 [SMILES: *c1ncc2occ(*)c2n1]

wherein “*” indicates the atom bonded to R⁴. In some embodiments, Ring L is optionally substituted

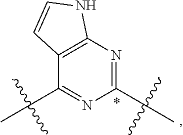 [SMILES: *c1nc(*)c2cc[nH]c2n1]

wherein “*” indicates the atom bonded to R⁴. In some embodiments, Ring L is optionally substituted

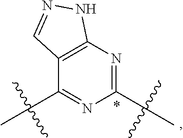 [SMILES: *c1nc(*)c2cn[nH]c2n1]

wherein “*” indicates the atom bonded to R⁴. In some embodiments, Ring L is optionally substituted

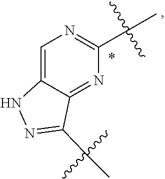 [SMILES: *c1ncc2[nH]nc(*)c2n1]

wherein “*” indicates the atom bonded to R⁴. In some embodiments, Ring L is optionally substituted

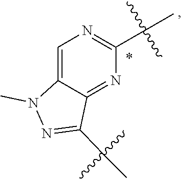 [SMILES: *c1ncc2c(n1)c(*)nn2C]

wherein “*” indicates the atom bonded to R⁴. In some embodiments, Ring L is optionally substituted

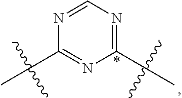 [SMILES: *c1ncnc(*)n1]

wherein “*” indicates the atom bonded to R⁴. In some embodiments, Ring L is optionally substituted

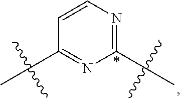 [SMILES: *c1ccnc(*)n1]

wherein “*” indicates the atom bonded to R⁴. In some embodiments, Ring L is optionally substituted

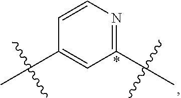 [SMILES: *c1ccnc(*)c1]

wherein “*” indicates the atom bonded to R⁴. In some embodiments, Ring L is optionally substituted

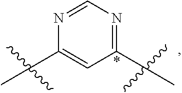 [SMILES: *c1cc(*)ncn1]

wherein “*” indicates the atom bonded to R⁴. In some embodiments, Ring L is optionally substituted

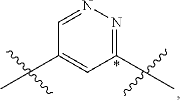 [SMILES: *c1cnnc(*)c1]

wherein “*” indicates the atom bonded to R⁴. In some embodiments, Ring L is optionally substituted

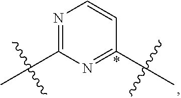 [SMILES: *c1ccnc(*)n1]

wherein “*” indicates the atom bonded to R⁴. In some embodiments, Ring L is optionally substituted

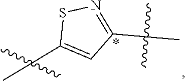 [SMILES: *c1cc(*)sn1]

wherein “*” indicates the atom bonded to R⁴. In some embodiments, Ring L is optionally substituted

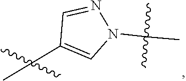 [SMILES: *c1cnn(*)c1]

wherein the nitrogen atom is boned to R⁴.

In some embodiments, Ring L is bonded to L^{b} at a monocyclic ring unit that is not bonded to R⁴.

### L^{b}

In some embodiments, L^{b} is L as described herein. In some embodiments, L^{b} is -L^{b1}-L^{b2}-L^{b3}-L^{b4}-L^{b5}-, wherein each of L^{b1}, L^{b2}, L^{b3}, L^{b4} and L^{b5} is independently L′ as described herein. In some embodiments, L^{b1} is bonded to Ring L.

In some embodiments, L^{b} is —C(R′)₂—N(R′)—C(O)— wherein each R′ is independently as described herein. In some embodiments, L^{b} is —C(R′)₂—N(R′)—C(O)—C≡C— wherein each R′ is independently as described herein. In some embodiments, L^{b} is —C(R′)₂—N(R′)—C(O)—C(R′)₂—OS(O)₂— wherein each R′ is independently as described herein. In some embodiments, L^{b} is —C(R′)₂—N(R′)—C(O)—C(R′)₂—OS(O)₂—O— wherein each R′ is independently as described herein. In some embodiments, —C(R′)₂— is bonded to L^{R}. In some embodiments, L^{b} is —CH₂—N(R′)—C(O)—, wherein the —CH₂— is optionally substituted and R′ is as described herein. In some embodiments, L^{b} is —CH₂—N(R′)—C(O)—C≡C—, wherein the —CH₂— is optionally substituted and R′ is as described herein. In some embodiments, L^{b} is —CH₂—N(R′)—C(O)—C(R′)₂—OS(O)₂—, wherein the —CH₂— is optionally substituted and each R′ is independently as described herein. In some embodiments, L^{b} is —CH₂—N(R′)—C(O)—C(R′)₂—OS(O)₂—O—, wherein the —CH₂— is optionally substituted and each R′ is independently as described herein. In some embodiments, —CH₂— is bonded to L^{R}. In some embodiments, -L^{R}-L^{b}- does not contain —C(R′)₂—N(R′)—C(O)—C≡C— wherein each R′ is independently as described herein. In some embodiments, -L^{R}-L^{b}- does not contain —C(R′)₂—N(R′)—C(O)—C(R′)₂—OS(O)₂— wherein each R′ is independently as described herein. In some embodiments, -L^{R}-L^{b}- does not contain —C(R′)₂—N(R′)—C(O)—C(R′)₂—OS(O)₂—O— wherein each R′ is independently as described herein. In some embodiments, -L^{R}-L^{b}- does not contain —C(R′)₂—N(R′)—C(O)—C(R′)₂— wherein each R′ is independently as described herein. In some embodiments, —C(R′)₂— is bonded to R⁴.

In some embodiments, L^{b} is -Cy- as described here.

In some embodiments, L^{b} is optionally substituted phenyl. In some embodiments, L^{b} is phenyl, optionally substituted with —OMe or halogen (e.g., —F, —Cl, —Br, or —I). In some embodiments, L^{b} is phenyl. In some embodiments, L^{b} is

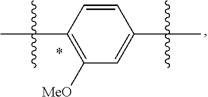 [SMILES: *c1ccc(*)c(OC)c1]

wherein “*” represents the point of attachment to L^{R}. In some embodiments, L^{b} is phenyl. In some embodiments, L^{b} is

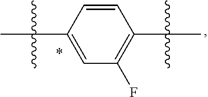 [SMILES: *c1ccc(*)c(F)c1]

wherein “*” represents the point of attachment to L^{R}. In some embodiments, L^{b} is

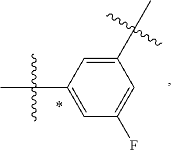 [SMILES: *c1cc(*)cc(F)c1]

wherein “*” represents the point of attachment to L^{R}. In some embodiments, R^{b} is —H, and L^{b} is

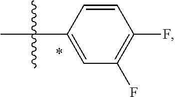 [SMILES: *c1ccc(F)c(F)c1]

wherein “*” represents the point of attachment to L^{R}. In some embodiments, R^{b} is —H, and L^{b} is

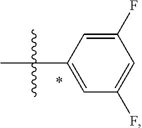 [SMILES: *c1cc(F)cc(F)c1]

wherein “*” represents the point of attachment to L^{R}. In some embodiments, L^{b} is

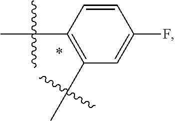 [SMILES: *c1ccc(F)cc1*]

wherein “*” represents the point of attachment to L^{R}.

In some embodiments, L^{b} a bivalent optionally substituted 5- or 6-membered heteroaryl ring having 1-4 (e.g., 0, 1, 2, 3, or 4) heteroatoms. In some embodiments, L^{b} is a bivalent optionally substituted 5- or 6-membered heteroaryl ring having 1 nitrogen heteroatom. In some embodiments, L^{b} is a bivalent optionally substituted pyridinyl. In some embodiments, L^{b} is a bivalent pyridinyl, optionally substituted with halogen (e.g., (—F, —Cl, —Br, or —I) or —OR [symbol] , wherein R [symbol] is C₁₋₆ aliphatic (e.g., methyl, ethyl, n-propyl, s-propyl, or isopropyl). In some embodiments, L^{b} is

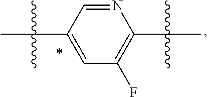 [SMILES: *c1cnc(*)c(F)c1]

wherein “*” represents the point of attachment to L^{R}. In some embodiments, L^{b} is

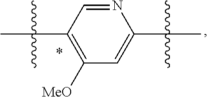 [SMILES: *c1cc(OC)c(*)cn1]

wherein “*” represents the point of attachment to L^{R}. In some embodiments, L^{b} is a bivalent pyridinyl. In some embodiments, L^{b} is

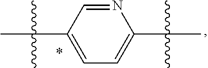 [SMILES: *c1ccc(*)nc1]

wherein “*” represents the point of attachment to L^{R}. In some embodiments, L^{b} is

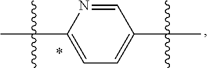 [SMILES: *c1ccc(*)nc1]

wherein “*” represents the point of attachment to L^{R}. In some embodiments, L^{b} is a bivalent optionally substituted 5- or 6-membered heteroaryl ring having 2 nitrogen heteroatoms. In some embodiments, L^{b} is a bivalent optionally substituted pyrimidinyl. In some embodiments, L^{b} is a bivalent pyrimidinyl. In some embodiments, L^{b} is

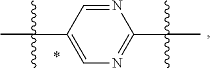 [SMILES: *c1cnc(*)nc1]

wherein “*” represents the point of attachment to L^{R}. In some embodiments, L^{b} is a bivalent optionally substituted pyridazinyl. In some embodiments, L^{b} is a bivalent pyridazinyl. In some embodiments, L^{b} is

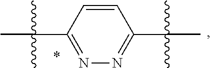 [SMILES: *c1ccc(*)nn1]

wherein “*” represents the point of attachment to L^{R}. In some embodiments, L^{b} is a bivalent optionally substituted 5- or 6-membered heteroaryl ring having 1 nitrogen and 1 sulfur heteroatom. In some embodiments, L^{b} is a bivalent optionally substituted thiazolyl. In some embodiments, L^{b} is a bivalent thiazolyl. In some embodiments, L^{b} is

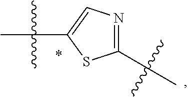 [SMILES: *c1cnc(*)s1]

wherein “*” represents the point of attachment to L^{R}.

In some embodiments, L^{b} is a bivalent optionally substituted 6- to 12-membered cycloalkyl ring. In some embodiments, L^{b} is a bivalent optionally substituted 5- to 6-membered cycloalkyl ring. In some embodiments, L^{b} is a bivalent optionally substituted 6-membered cycloalkyl ring. In some embodiments, L^{b} is a bivalent optionally substituted 5-membered cycloalkyl ring. In some embodiments, L^{b} is a bivalent cyclohexanyl, optionally substituted with C₁₋₆ aliphatic (e.g., methyl, ethyl, n-propyl, s-propyl, or isopropyl) or halogen (e.g., —F, —Cl, —Br, —I). In some embodiments, L^{b} is

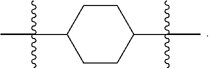 [SMILES: *C1CCC(*)CC1]

In some embodiments, L^{b} is a bivalent optionally substituted 6- to 12-membered heterocyclyl ring having 1-4 (e.g., 0, 1, 2, 3, or 4) heteroatoms. In some embodiments, L^{b} is a bivalent optionally substituted 6- to 12-membered heterocyclyl ring having 1-4 (e.g., 0, 1, 2, 3, or 4) heteroatoms selected from nitrogen, oxygen, or sulphur. In some embodiments, L^{b} is a bivalent optionally substituted 9- to 10-membered heteroaryl ring having 1-4 (e.g., 0, 1, 2, 3, or 4) heteroatoms selected from nitrogen, oxygen, or sulphur. In some embodiments, L^{b} is a bivalent optionally substituted 9-membered heteroaryl ring having 1-4 (e.g., 0, 1, 2, 3, or 4) heteroatoms selected from nitrogen, oxygen, or sulphur. In some embodiments, L^{b} is a bivalent optionally substituted 9-membered heteroaryl ring having 1 oxygen heteroatoms.

In some embodiments, L^{b} is a bivalent optionally substituted 2,3-dihydrobenzofuranyl. In some embodiments, L^{b} is a bivalent 2,3-dihydrobenzofuranyl, optionally substituted with C₁₆ aliphatic (e.g., methyl, ethyl, n-propyl, s-propyl, or isopropyl) or halogen (e.g., —F, —Cl, —Br, -1). In some embodiments, L^{b} is a bivalent 2,3-dihydrobenzofuranyl. In some embodiments, L^{b} is

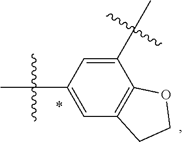 [SMILES: *c1cc(*)c2c(c1)CCO2]

wherein “*” represents the point of attachment to L^{R}. In some embodiments, R^{b} is —H, and L^{b} is

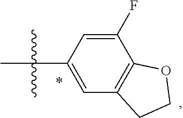 [SMILES: *c1cc(F)c2c(c1)CCO2]

wherein “*” represents the point of attachment to L^{R}.

In some embodiments, L^{b} is a bivalent optionally substituted 10-membered heteroaryl ring having 1-4 (e.g., 0, 1, 2, 3, or 4) heteroatoms selected from nitrogen, oxygen, or sulphur. In some embodiments, L^{b} is a bivalent optionally substituted 10-membered heteroaryl ring having 1 oxygen heteroatoms. In some embodiments, L^{b} is a bivalent optionally substituted chromanyl. In some embodiments, R^{b} is —H, and L^{b} is chromanyl. In some embodiments, R^{b} is —H, and L^{b} is

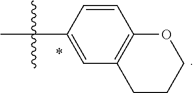 [SMILES: *c1ccc2c(c1)CCCO2]

In some embodiments, R^{b} is —H, and L^{b} is

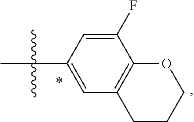 [SMILES: *c1cc(F)c2c(c1)CCCO2]

wherein “*” represents the point of attachment to L^{R}.

In some embodiments, L^{b} is a bivalent optionally substituted 10- to 15-membered tricyclic heterocyclyl ring having 1-4 (e.g., 0, 1, 2, 3, or 4) heteroatoms. In some embodiments, L^{b} is a bivalent optionally substituted 10- to 15-membered tricyclic heterocyclyl ring having 1-4 (e.g., 0, 1, 2, 3, or 4) heteroatoms selected from oxygen, nitrogen, or sulfur. In some embodiments, L^{b} is a bivalent optionally substituted 10- to 15-membered tricyclic heterocyclyl having 1-4 (e.g., 0, 1, 2, 3, or 4) heteroatoms selected from oxygen, nitrogen, or sulfur, wherein the tricyclic heterocyclyl comprises two spiro rings and two fused rings. In some embodiments, L^{b} is a bivalent optionally substituted 12-membered tricyclic heterocyclyl ring having 1-4 (e.g., 0, 1, 2, 3, or 4) heteroatoms selected from oxygen, nitrogen, or sulfur. In some embodiments, L^{b} is a bivalent optionally substituted spiro[chromane-4,1′-cyclopropanyl. In some embodiments, L^{b} is a bivalent spiro[chromane-4,1′-cyclopropanyl. In some embodiments, R^{b} is —H, and L^{b} is spiro[chromane-4,1′-cyclopropanyl. In some embodiments, R^{b} is —H, and L^{b} is

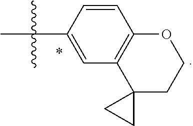 [SMILES: *c1ccc2c(c1)C1(CCO2)CC1]

In some embodiments, L^{b} is a bivalent optionally substituted 6- to 12-membered heteroaryl ring having 1-4 (e.g., 0, 1, 2, 3, or 4) heteroatoms. In some embodiments, L^{b} is a bivalent optionally substituted 6- to 12-membered heteroaryl ring having 1-4 (e.g., 0, 1, 2, 3, or 4) heteroatoms selected from nitrogen, oxygen, or sulphur. In some embodiments, L^{b} is a bivalent optionally substituted 9- to 10-membered heteroaryl ring having 1-4 (e.g., 0, 1, 2, 3, or 4) heteroatoms selected from nitrogen, oxygen, or sulphur. In some embodiments, L^{b} is a bivalent optionally substituted 9-membered heteroaryl ring having 1-4 (e.g., 0, 1, 2, 3, or 4) heteroatoms selected from nitrogen, oxygen, or sulphur. In some embodiments, L^{b} is a bivalent optionally substituted 9-membered heteroaryl ring having 2 nitrogen heteroatoms.

In some embodiments, L^{b} is a bivalent optionally substituted imidazopyridinyl. In some embodiments, L^{b} is a bivalent optionally substituted imidazo[1,5-a]pyridinyl. In some embodiments, L^{b} is a bivalent imidazo[1,5-a]pyridinyl, optionally substituted with C₁₆ aliphatic (e.g., methyl, ethyl, n-propyl, s-propyl, or isopropyl) or halogen (e.g., —F, —Cl, —Br, —I). In some embodiments, L^{b} is a bivalent imidazo[1,5-a]pyridinyl. In some embodiments, L^{b} is

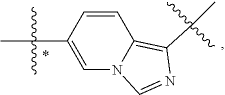 [SMILES: *c1ccc2c(*)ncn2c1]

wherein “*” represents the point of attachment to L^{R}.

In some embodiments, L^{b} is a bivalent optionally substituted indazolyl. In some embodiments, L^{b} a bivalent indazolyl, optionally substituted with C₁₋₆ aliphatic (e.g., methyl, ethyl, n-propyl, s-propyl, or isopropyl) or halogen (e.g., —F, —Cl, —Br, —I). In some embodiments, L^{b} is optionally substituted

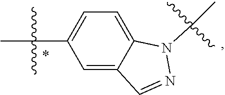 [SMILES: *c1ccc2c(cnn2*)c1]

wherein “*” represents the point of attachment to L^{R}. In some embodiments, L^{b} is optionally substituted

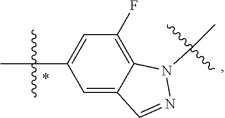 [SMILES: *c1cc(F)c2c(cnn2*)c1]

wherein “*” represents the point of attachment to L^{R}.

In some embodiments, L^{b} is a bivalent optionally substituted benzoimidazolyl. In some embodiments, L^{b} is a bivalent optionally substituted benzo[d]imidazolyl. In some embodiments, L^{b} is a bivalent benzo[d]imidazolyl, optionally substituted with C₁₋₆ aliphatic (e.g., methyl, ethyl, n-propyl, s-propyl, or isopropyl) or halogen (e.g., —F, —Cl, —Br, —I). In some embodiments, L^{b} is optionally substituted

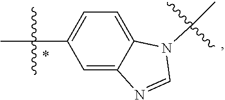 [SMILES: *c1ccc2c(c1)ncn2*]

wherein “*” represents the point of attachment to L^{R}. In some embodiments, L^{b} is optionally substituted

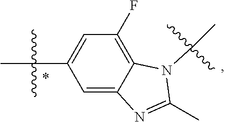 [SMILES: *c1cc(F)c2c(c1)nc(C)n2*]

wherein “*” represents the point of attachment to L^{R}. In some embodiments, L^{b} is optionally substituted

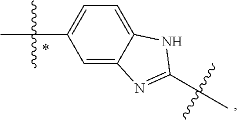

wherein represents the point of attachment to L^{R}. In some embodiments, L^{b} is optionally substituted

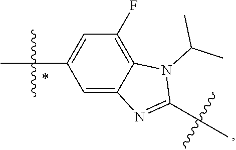 [SMILES: *c1cc(F)c2c(c1)nc(*)n2C(C)C]

wherein “*” represents the point of attachment to L^{R}.

In some embodiments, L^{b} is a bivalent optionally substituted 9-membered heteroaryl ring having 3 nitrogen heteroatoms. In some embodiments, L^{b} is a bivalent optionally substituted pyrazolylpyrimidinyl. In some embodiments, L^{b} is a bivalent optionally substituted pyrazolyl[1,5-a]pyrimidinyl. In some embodiments, L^{b} is a bivalent pyrazolyl[1,5-a]pyrimidinyl, optionally substituted with C₁₋₆ aliphatic (e.g., methyl, ethyl, n-propyl, s-propyl, or isopropyl) or halogen (e.g., —F, —Cl, —Br, —I).

In some embodiments, L^{b} is optionally substituted

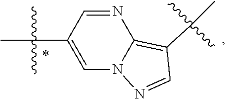 [SMILES: *c1cnc2c(*)cnn2c1]

wherein “*” represents the point of attachment to L^{R}. In some embodiments, L^{b} is

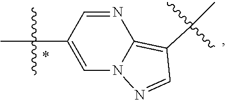 [SMILES: *c1cnc2c(*)cnn2c1]

wherein “*” represents the point of attachment to L^{R}.

In some embodiments, L^{b} is *—N(R′)-Cy-, wherein “*” represents the point of attachment to L^{R}. In some embodiments, L^{b} is *—N(H)-Cy-, wherein “*” represents the point of attachment to L^{R}. In some embodiments, L^{b} is

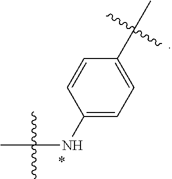 [SMILES: *Nc1ccc(*)cc1]

In some embodiments, L^{b} is

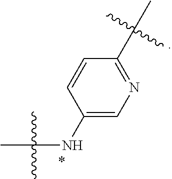 [SMILES: *Nc1ccc(*)nc1]

In some embodiments, L^{b} is

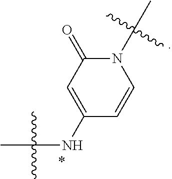 [SMILES: *Nc1ccn(*)c(=O)c1]

### L^{b1}

In some embodiments, L^{b1} is L′ as described herein. In some embodiments, L^{b1} is a covalent bond. In some embodiments, L^{b1} is not a covalent bond. In some embodiments, L^{b1} is or comprises optionally substituted —CH₂—. In some embodiments, L^{b1} is or comprises —CH₂—. In some embodiments, L^{b} is or comprises —C(R′)₂— wherein each R′ is independently as described herein. In some embodiments, the two R′ of —C(R′)₂— are taken together with the carbon atom to which they are attached to form an optionally substituted 3-10 (e.g., 3-9, 5-10, 3, 4, 5, 6, 7, 8, 9, 10, etc.) membered saturated or partially unsaturated ring having 0-4 (e.g., 1-4, 0, 1, 2, 3, 4, etc.) heteroatoms. In some embodiments, the two R′ of —C(R′)₂— are taken together with the carbon atom to which they are attached to form an optionally substituted 3-10 (e.g., 3-9, 5-10, 3, 4, 5, 6, 7, 8, 9, 10, etc.) membered saturated ring having 0-2 (e.g., 1-2, 0, 1, 2, etc.) heteroatoms. In some embodiments, L^{b1} is or comprises —CHR′— wherein R′ is as described herein. In some embodiments, L^{b1} is or comprises —CHR′—, wherein R′ is optionally substituted aryl or heteroaryl. In some embodiments, L^{b1} is or comprises —CHR′—, wherein R′ is optionally substituted phenyl. In some embodiments, L^{b1} is or comprises —N(R′)— wherein R′ is as described herein. In some embodiments, L^{b1} is or comprises —NH—. In some embodiments, L^{b1} is or comprises —C(O)—. In some embodiments, L^{b1} is or comprises -Cy- as described herein. In some embodiments, L^{b1} is or comprises -Cy-, wherein -Cy- is optionally substituted bivalent saturated or partially unsaturated 3-7 (e.g., 3-6, 3, 4, 5, 6, 7, etc.) membered ring having 0-2 (e.g., 1-2, 0, 1, 2, etc.) heteroatoms. In some embodiments, L^{b1} is or comprises -Cy-, wherein -Cy- is optionally substituted bivalent saturated 6-membered ring having 1-2 heteroatoms that are nitrogen.

### L^{b2}

In some embodiments, L^{b2} is L^{z} as described herein. In some embodiments, L^{b2} is a covalent bond. In some embodiments, L^{b2} is not a covalent bond. In some embodiments, L^{b2} is or comprises optionally substituted —CH₂—. In some embodiments, L^{b2} is or comprises —CH₂—. In some embodiments, L^{b2} is or comprises —C(R′)₂— wherein each R′ is independently as described herein. In some embodiments, the two R′ of —C(R′)₂— are taken together with the carbon atom to which they are attached to form an optionally substituted 3-10 (e.g., 3-9, 5-10, 3, 4, 5, 6, 7, 8, 9, 10, etc.) membered saturated or partially unsaturated ring having 0-4 (e.g., 1-4, 0, 1, 2, 3, 4, etc.) heteroatoms. In some embodiments, the two R′ of —C(R′)₂— are taken together with the carbon atom to which they are attached to form an optionally substituted 3-10 (e.g., 3-9, 5-10, 3, 4, 5, 6, 7, 8, 9, 10, etc.) membered saturated ring having 0-2 (e.g., 1-2, 0, 1, 2, etc.) heteroatoms. In some embodiments, L^{b2} is or comprises —CHR′— wherein R′ is as described herein. In some embodiments, L^{b2} is or comprises —CHR′—, wherein R′ is optionally substituted aryl or heteroaryl. In some embodiments, L^{b2} is or comprises —CHR′—, wherein R′ is optionally substituted phenyl. In some embodiments, L^{b2} is or comprises —N(R′)— wherein R′ is as described herein. In some embodiments, L^{b2} is or comprises —N(R′)— wherein R′ is or comprises an optionally substituted ring, e.g., cycloaliphatic (e.g., optionally substituted saturated or partially unsaturated 3-10 membered (e.g., 3-9, 5-10, 3, 4, 5, 6, 7, 8, 9, 10, etc.) cycloaliphatic), cycloheteroaliphatic (e.g., optionally substituted saturated or partially unsaturated 3-10 membered (e.g., 3-9, 5-10, 3, 4, 5, 6, 7, 8, 9, 10, etc.) ring having 1-4 (e.g., 1-3, 1, 2, 3, 4, etc.) heteroatoms), aryl (e.g., optionally substituted 6-10 membered (e.g., 6, 8, 9, 10, etc.) aryl, heteroaryl (e.g., optionally substituted 5-10 membered (e.g., 5-6, 5-9, 5, 7, 8, 9, 10, etc.) heteroaryl having 1-4 (e.g., 1-3, 1, 2, 3, 4, etc.) heteroatoms), etc. In some embodiments, L^{b2} is or comprises —N(R′)— wherein R′ is optionally substituted 5-10 (e.g., 6-10, 5-9, 5, 6, 7, 8, 9, 10, etc.) membered aryl or heteroaryl having 0-4 (e.g., 1-4, 0, 1,2, 3, 4, etc.) heteroatoms. In some embodiments, L^{b2} is or comprises —N(R′)— wherein R′ is optionally substituted phenyl. In some embodiments, L^{b2} is or comprises —NH—. In some embodiments, L^{b2} is or comprises —C(O)N(R′)— wherein R′ is as described herein. In some embodiments, L^{b2} is or comprises —C(O)N(R′)— wherein R′ is optionally substituted 5-10 (e.g., 6-10, 5-9, 5, 6, 7, 8, 9, 10, etc.) membered aryl or heteroaryl having 0-4 (e.g., 1-4, 0, 1,2, 3, 4, etc.) heteroatoms. In some embodiments, L^{b}a is or comprises —C(O)N(R′)— wherein R′ is optionally substituted phenyl. In some embodiments, L^{b2} is or comprises —C(O)NH—. In some embodiments, L^{b2} is or comprises —C(O)—. In some embodiments, L^{b2} is or comprises -Cy- as described herein. In some embodiments, L^{b2} is or comprises -Cy- as described herein. In some embodiments, L² is or comprises -Cy-, wherein -Cy- is optionally substituted bivalent saturated or partially unsaturated 3-7 (e.g., 3-6, 3, 4, 5, 6, 7, etc.) membered ring having 0-2 (e.g., 1-2, 0, 1, 2, etc.) heteroatoms. In some embodiments, L^{b2} is or comprises -Cy-, wherein -Cy- is optionally substituted bivalent saturated 6-membered ring having 1-2 heteroatoms that are nitrogen. In some embodiments, -Cy- is an optionally substituted bicyclic ring, wherein at least one monocyclic ring is an optionally substituted phenyl ring. In some embodiments, -Cy- is an optionally substituted bicyclic ring, wherein at least one monocyclic ring is an optionally substituted 5-6 membered heteroaryl ring having 1-3 (e.g., 1-2, 1, 2, 3, etc.) heteroatoms. In some embodiments, -Cy- is an optionally substituted bicyclic ring, wherein each monocyclic ring is independently an optionally substituted phenyl or 5-6 membered heteroaryl ring having 1-3 (e.g., 1-2, 1, 2, 3, etc.) heteroatoms. In some embodiments, -Cy- is an optionally substituted bicyclic ring, wherein one monocyclic ring is an optionally substituted phenyl or 5-6 membered heteroaryl ring having 1-3 (e.g., 1-2, 1, 2, 3, etc.) heteroatoms, and the other monocyclic ring is an optionally substituted non-aromatic 3-10 (e.g., 3-9, 4-10, 5-10, 3, 4, 5, 6, 7, 8, 9, 10, etc.) membered ring having 1-4 (e.g., 1-2, 1-3, 1, 2, 3, 4, etc.) heteroatoms. In some embodiments, -Cy- is an optionally substituted bicyclic ring, wherein one monocyclic ring is an optionally substituted phenyl or 5-6 membered heteroaryl ring having 1-3 heteroatoms, and the other monocyclic ring is an optionally substituted non-aromatic 5-6 membered ring having 1-3 (e.g., 1-2, 1, 2, 3, etc.) heteroatoms. In some embodiments, -Cy- is an optionally substituted 3-10 (e.g., 3-9, 5-10, 3, 4, 5, 6, 7, 8, 9, 10, etc.) membered bivalent cycloaliphatic ring. In some embodiments, -Cy- is an optionally substituted 6-membered cycloalkyl ring. In some embodiments, -Cy- is optionally substituted phenylene. In some embodiments, -Cy- is optionally substituted 1, 4-phenylene. In some embodiments, -Cy- is 1, 4-phenylene. In some embodiments, -Cy- is optionally substituted bivalent cyclohexyl. In some embodiments, -Cy- is optionally substituted 1, 4-cyclohexyl. In some embodiments, -Cy- is 1, 4-cyclohexyl. In some embodiments, -Cy- is an optionally substituted bivalent piperidyl ring. In some embodiments, -Cy- is a bivalent piperidyl ring. In some embodiments, a bivalent piperidyl ring is bivalent 1,2-piperidyl. In some embodiments, a bivalent piperidyl ring is bonded to L^{b1} at 1′. In some embodiments, a bivalent piperidyl ring is bivalent 1,4-piperidyl. In some embodiments, -Cy- is an optionally substituted bivalent piperazinyl ring. In some embodiments, -Cy- is a bivalent piperazinyl ring. In some embodiments, a bivalent piperidyl ring is bivalent 1,2-piperazinyl. In some embodiments, a bivalent piperidyl ring is bivalent 1,4-piperazinyl. In some embodiments, -Cy- is an optionally substituted 5-6 membered bivalent heteroaryl ring. In some embodiments, -Cy- is an optionally substituted 5-membered bivalent heteroaryl ring. In some embodiments, -Cy- is an optionally substituted 6-membered bivalent heteroaryl ring having 1 or 3 nitrogen atoms. In some embodiments, -Cy- is an optionally substituted 6-membered bivalent heteroaryl ring having a nitrogen atom. In some embodiments, -Cy- is an optionally substituted bivalent pyridinyl ring. In some embodiments, -Cy- is a bivalent pyridinyl ring. In some embodiments, a bivalent pyridinyl ring is a 2, 5-bivalent pyridinyl ring. In some embodiments, a bivalent pyridinyl ring is bonded to L^{b1} at 2′. In some embodiments, a bivalent pyridinyl ring is bonded to L^{b1} at 5′. In some embodiments, a bivalent pyridinyl ring is a 3, 5-bivalent pyridinyl ring. In some embodiments, a bivalent pyridinyl ring is bonded to L^{b1} at 3′. In some embodiments, a bivalent pyridinyl ring is bonded to L^{b1} at 5′.

### L^{b3}

In some embodiments, L^{b3} is L′ as described herein. In some embodiments, L^{b3} is a covalent bond. In some embodiments, L^{b3} is not a covalent bond. In some embodiments, L^{b3} is or comprises optionally substituted —CH₂—. In some embodiments, L¹³ is or comprises —CH₂—. In some embodiments, L^{b3} is or comprises —C(R′)₂— wherein each R′ is independently as described herein. In some embodiments, the two R′ of —C(R′)₂— are taken together with the carbon atom to which they are attached to form an optionally substituted 3-10 (e.g., 3-9, 5-10, 3, 4, 5, 6, 7, 8, 9, 10, etc.) membered saturated or partially unsaturated ring having 0-4 (e.g., 1-4, 0, 1, 2, 3, 4, etc.) heteroatoms. In some embodiments, the two R′ of —C(R′)₂— are taken together with the carbon atom to which they are attached to form an optionally substituted 3-10 (e.g., 3-9, 5-10, 3, 4, 5, 6, 7, 8, 9, 10, etc.) membered saturated ring having 0-2 (e.g., 1-2, 0, 1, 2, etc.) heteroatoms. In some embodiments, L^{b3} is or comprises —CHR′— wherein R′ is as described herein. In some embodiments, L^{b3} is or comprises —CHR′—, wherein R′ is optionally substituted aryl or heteroaryl. In some embodiments, L^{b3} is or comprises —CHR′—, wherein R′ is optionally substituted phenyl. In some embodiments, L^{b3} is or comprises —N(R′)— wherein R′ is as described herein. In some embodiments, L^{b3} is or comprises —NH—. In some embodiments, L^{b3} is or comprises —C(O)—. In some embodiments, L^{b3} is or comprises -Cy- as described herein. In some embodiments, L^{b3} is or comprises —O—. In some embodiments, L^{b3} is or comprises —S—. L^{b4}

In some embodiments, L^{b4} is L′ as described herein. In some embodiments, L^{b4} is a covalent bond. In some embodiments, L^{b4} is not a covalent bond. In some embodiments, L^{b4} is —C≡C—. In some embodiments, L^{b4} is or comprises optionally substituted —CH₂—. In some embodiments, L^{b4} is or comprises —CH₂—. In some embodiments, L^{b4} is or comprises optionally substituted —CH₂—O—. In some embodiments, L^{b4} is or comprises —CH₂—O—. In some embodiments, L^{b4} is or comprises —C(R′)₂— wherein each R′ is independently as described herein. In some embodiments, the two R′ of —C(R′)₂— are taken together with the carbon atom to which they are attached to form an optionally substituted 3-10 (e.g., 3-9, 5-10, 3, 4, 5, 6, 7, 8, 9, 10, etc.) membered saturated or partially unsaturated ring having 0-4 (e.g., 1-4, 0, 1, 2, 3, 4, etc.) heteroatoms. In some embodiments, the two R′ of —C(R′)₂— are taken together with the carbon atom to which they are attached to form an optionally substituted 3-10 (e.g., 3-9, 5-10, 3, 4, 5, 6, 7, 8, 9, 10, etc.) membered saturated ring having 0-2 (e.g., 1-2, 0, 1, 2, etc.) heteroatoms. In some embodiments, L^{b4} is or comprises —CHR′— wherein R′ is as described herein. In some embodiments, L^{b4} is or comprises —CHR′—, wherein R′ is optionally substituted aryl or heteroaryl. In some embodiments, L^{b4} is or comprises —CHR′—, wherein R′ is optionally substituted phenyl. In some embodiments, L^{b4} is or comprises —N(R′)— wherein R′ is as described herein. In some embodiments, L^{b4} is or comprises —NH—. In some embodiments, L^{b4} is or comprises —C(O)—. In some embodiments, L^{b4} is or comprises -Cy- as described herein. In some embodiments, wherein -Cy- is an optionally substituted 3-10 (e.g., 4-10, 5-10, 3, 4, 5, 6, 7, 8, 9, 10, etc.) membered bivalent cycloaliphatic ring. In some embodiments, -Cy- is an optionally substituted 6-membered cycloalkyl ring. In some embodiments, -Cy- is optionally substituted bivalent cyclohexyl. In some embodiments, -Cy- is an optionally substituted 3-10 (e.g., 4-10, 5-10, 3, 4, 5, 6, 7, 8, 9, 10, etc.) membered bivalent heterocyclyl ring having 1-3 (e.g., 1-2, 1, 2, 3, etc.) heteroatoms. In some embodiments, -Cy- is an optionally substituted 6-membered bivalent heterocyclyl ring having 1-3 (e.g., 1-2, 1, 2, 3, etc.) heteroatoms. In some embodiments, -Cy- is an optionally substituted 5-membered bivalent heterocyclyl ring having 1 or 2 heteroatoms. In some embodiments, -Cy- is an optionally substituted saturated ring. In some embodiments, -Cy- is an optionally substituted 5-6 membered bivalent heteroaryl ring having 1-3 (e.g., 1-2, 1, 2, 3, etc.) heteroatoms. In some embodiments, -Cy- is optionally substituted bivalent phenylene. In some embodiments, -Cy- is optionally substituted bivalent 1,4-phenylene. In some embodiments, -Cy- is bivalent 1, 4-phenylene. In some embodiments, -Cy- is optionally substituted bivalent naphthyl. In some embodiments, L^{b4} is or comprises —O—. In some embodiments, L^{b4} is or comprises —S—.

### L^{b5}

In some embodiments, L^{b5} is L′ as described herein. In some embodiments, L^{b5} is a covalent bond. In some embodiments, L^{b5} is not a covalent bond. In some embodiments, L^{b5} is or comprises optionally substituted —CH₂—. In some embodiments, L^{b5} is or comprises —CH₂—. In some embodiments, L^{b5} is or comprises optionally substituted —CH₂O—. In some embodiments, L^{b5} is or comprises —CH₂O—. In some embodiments, L^{b5} is or comprises —C(R′)₂— wherein each R′ is independently as described herein. In some embodiments, the two R′ of —C(R′)₂— are taken together with the carbon atom to which they are attached to form an optionally substituted 3-10 (e.g., 3-9, 5-10, 3, 4, 5, 6, 7, 8, 9, 10, etc.) membered saturated or partially unsaturated ring having 0-4 (e.g., 1-4, 0, 1, 2, 3, 4, etc.) heteroatoms. In some embodiments, the two R′ of —C(R′)₂— are taken together with the carbon atom to which they are attached to form an optionally substituted 3-10 (e.g., 3-9, 5-10, 3, 4, 5, 6, 7, 8, 9, 10, etc.) membered saturated ring having 0-2 (e.g., 1-2, 0, 1, 2, etc.) heteroatoms. In some embodiments, L^{b5} is or comprises —CHR′— wherein R′ is as described herein. In some embodiments, L^{b5} is or comprises —CHR′—, wherein R′ is optionally substituted aryl or heteroaryl. In some embodiments, L^{b5} is or comprises —CHR′—, wherein R′ is optionally substituted phenyl. In some embodiments, L^{b5} is or comprises —N(R′)— wherein R′ is as described herein. In some embodiments, L^{b5} is or comprises —NH—. In some embodiments, L^{b5} is or comprises —C(O)—. In some embodiments, L^{b5} is or comprises -Cy- as described herein. In some embodiments, L^{b5} is or comprises —O—. In some embodiments, L^{b5} is or comprises —S—. In some embodiments, wherein L^{b5} is or comprises —S(O)₂—. In some embodiments, L^{b5} is or comprises —OS(O)₂—.

### L′

In some embodiments, L′ is a covalent bond, or an optionally substituted bivalent C₁₋₂ aliphatic or heteroaliphatic having 1-2 heteroatoms, wherein one or more methylene units are optionally and independently replaced with —C(R′)₂—, -Cy-, —O—, —S—, —N(R′)—, C(O)—, C(S)—, —C(NR′)—, —C(O)N(R′)—, O—, —S—, —N(R′)—, —C(O)—, —C(S)—, —C(NR′)— —N(R′)C(O)N(R′)—, —N(R′)C(O)O—, —S(O)—, —S(O)₂—, —S(O)₂N(R′)—, —C(O)S—, or —C(O)O—. In some embodiments, L′ is a covalent bond. In some embodiments, L′ is an optionally substituted bivalent C₁₋₂ aliphatic or heteroaliphatic having 1-2 heteroatoms, wherein one or more methylene units are optionally and independently replaced with —C(R′)₂—, -Cy-, —O—, —S—, —N(R′)—, —C(O)—, —C(S)—, —C(NR′)—, —C(O)N(R′)—, O—, —S—, —N(R′)—, —C(O)—, —C(S)—, —C(NR′)— —N(R′)C(O)N(R′)—, —N(R′)C(O)O—, —S(O)—, —S(O)₂—, —S(O)₂N(R′)—, —C(O)S—, or —C(O)O—. In some embodiments, L′ is an optionally substituted bivalent C₁₋₂ aliphatic. In some embodiments, L′ is an optionally substituted bivalent C₁₋₂ alkylene wherein one or more methylene units are optionally and independently replaced with —C(R′)₂—, -Cy-, —O—, —S—, —N(R′)—, —C(O)—, —C(S)—, —C(NR′)—, —C(O)N(R′)—, O—, —S—, —N(R′)—, —C(O)—, —C(S)—, —C(NR′)— —N(R′)C(O)N(R′)—, —N(R′)C(O)O—, —S(O)—, —S(O)₂—, —S(O)₂N(R′)—, —C(O)S—, or —C(O)O—. In some embodiments, L′ is an optionally substituted bivalent —CH₂—CH₂— wherein one or more methylene units are optionally and independently replaced with —C(R′)₂—, -Cy-, —O—, —S—, —N(R′)—, —C(O)—, —C(S)—, —C(NR′)—, —C(O)N(R′)—, O—, —S—, —N(R′)—, —C(O)—, —C(S)—, —C(NR′)— —N(R′)C(O)N(R′)—, —N(R′)C(O)O—, —S(O)—, —S(O)₂—, —S(O)₂N(R′)—, —C(O)S—, or —C(O)O—. In some embodiments, L′ is or comprises optionally substituted —CH₂—. In some embodiments, L′ is or comprises —CH₂—. In some embodiments, L′ is or comprises optionally substituted —CH₂—CH₂—. In some embodiments, L′ is or comprises —CH₂—CH₂—. In some embodiments, a methylene unit is replaced with —C(R′)₂— wherein each R′ is independently as described herein. In some embodiments, a methylene unit is replaced with CHR′ wherein R′ is as described herein. In some embodiments, a methylene unit is replaced with —N(R′)— wherein R′ is as described herein. In some embodiments, a methylene unit is replaced with —C(O)N(R′)— wherein R′ is as described herein. In some embodiments, a methylene unit is replaced with -Cy- wherein Cy is as described herein. In some embodiments, a methylene unit is replaced with —C(O)— as described herein. In some embodiments, a methylene unit is replaced with —O— as described herein. In some embodiments, a methylene unit is replaced with —S— as described herein.

In some embodiments, L′ is L^{b1} as described herein. In some embodiments, L′ is L^{b2} as described herein. In some embodiments, L′ is L^{b3} as described herein. In some embodiments, L′ is L^{b4} as described herein. In some embodiments, L′ is L^{b5} as described herein.

### R^{b}

In some embodiments, R^{b} is R″ as described herein. In some embodiments, R^{b} is R′ as described herein. In some embodiments, R^{b} is R as described herein. In some embodiments, R^{b} is not hydrogen. In some embodiments, R^{b} is hydrogen.

In some embodiments, R^{b} is —CN. In some embodiments, R^{b} is —OR wherein R is as described herein. In some embodiments, R^{b} is —OH. In some embodiments, R^{b} is —OCH₃. In some embodiments, R^{b} is —OCH(CH₂CH₃)₂. In some embodiments, R^{b} is —OCH₂CH₂CH₂CH₃. In some embodiments, R^{b} is —OCH₂CH₂CH₂CH₂CH₂CH₃. In some embodiments, R^{b} is —CF₃. In some embodiments, R^{b} is —N(R′)₂. In some embodiments, R^{b} is —N(CH₂CH₃)₂. In some embodiments, R^{b} is an optionally substituted sulfone. In some embodiments, R^{b} is a sulfone. In some embodiments, R^{b} is —S(O₂)CH₃.

In some embodiments, R^{b} is optionally substituted C₁₋₂₀ (e.g., C₁₋₁₅, C₁₋₁₀, C₁₋₉, C₁₋₈, C₁₋₆, C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, etc.) aliphatic. In some embodiments, R^{b} is optionally substituted C₁₋₁₅ aliphatic. In some embodiments, R^{b} is optionally substituted C₁₋₁₀ aliphatic. In some embodiments, R^{b} is optionally substituted C₁₋₆ aliphatic. In some embodiments, an optionally substituted aliphatic is an optionally substituted alkyl. In some embodiments, R^{b} is optionally substituted C₁₋₆ alkyl. In some embodiments, R^{b} is optionally substituted methyl. In some embodiments, R^{b} is methyl. In some embodiments, R^{b} is optionally substituted ethyl. In some embodiments, R^{b} is ethyl. In some embodiments, R^{b} is —CH₂CF₃. In some embodiments, R^{b} is optionally substituted propyl. In some embodiments, R^{b} is optionally substituted isopropyl. In some embodiments, R^{b} is isopropyl. In some embodiments, R^{b} is optionally substituted —C₀₋₄alkyl-C≡CH. In some embodiments, R^{b} is propargyl. In some embodiments, R^{b} is optionally substituted C₃₋₂₀ (e.g., C₃₋₁₅, C₃₋₁₀, C₃₋₉, C₃₋₈, C₃₋₆, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, etc.) cycloaliphatic. In some embodiments, R^{b} is optionally substituted C₃₋₁₀ cycloaliphatic. In some embodiments, R^{b} is optionally substituted C₃₋₁₀ cycloalkyl. In some embodiments, R^{b} is optionally substituted cyclopropyl. In some embodiments, R^{b} is optionally substituted cyclobutyl. In some embodiments, R^{b} is optionally substituted cyclopentyl. In some embodiments, R^{b} is optionally substituted cyclohexyl. In some embodiments, R^{b} is optionally substituted C₁₋₂₀ (e.g., C₁₋₁₅, C₁₋₁₀, C₁₋₉, C₁₋₈, C₁₋₆, C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, etc.) heteroaliphatic having 1-10 (e.g., 1-5, 1-3, 1, 2, 3, 4, 5, 6, 7, 8, 9, 10, etc.) heteroatoms. In some embodiments, R^{b} is optionally substituted C₁₋₁₀ (e.g., C₁₋₉, C₁₋₈, C₁₋₆, C₁, C₂, C₃, C₄, C₅, C₆, C₇, C₈, C₉, C₁₀, etc.) heteroaliphatic having 1-5 (e.g., 1-4, 1-3, 1, 2, 3, 4, 5, etc.) heteroatoms. In some embodiments, R^{b} is optionally substituted C₆₋₂₀ (e.g., 6-10, 6, 8, 9, 10, 14, etc. membered) aryl. In some embodiments, R^{b} is optionally substituted phenyl. In some embodiments, R^{b} is phenyl. In some embodiments, R^{b} is optionally substituted naphthyl. In some embodiments, R^{b} is naphthyl. In some embodiments, R^{b} is optionally substituted 5-20 (e.g., 5-14, 5-10, 5, 6, 8, 9, 10, 14, etc.) membered heteroaryl having 1-10 (1-6, 1-5, 1-4, 1, 2, 3, 4, 5, 6, 7, 8, 9, 10, etc.) heteroatoms. In some embodiments, R^{b} is optionally substituted 5-10 membered heteroaryl having 1-5 (e.g., 1-4, 1-3, 1, 2, 3, 4, 5, etc.) heteroatoms. In some embodiments, R^{b} is optionally substituted 5-6 membered heteroaryl having 1-4 (e.g., 1-3, 1, 2, 3, 4, etc.) heteroatoms. In some embodiments, R^{b} is optionally substituted 5-6 membered heteroaryl having 1-4 (e.g., 1-3, 1, 2, 3, 4, etc.) nitrogen atoms. In some embodiments, R^{b} is optionally substituted 5-6 membered heteroaryl having 1 or 2 nitrogen atoms. In some embodiments, R^{b} is optionally substituted pyridyl. In some embodiments, R^{b} is optionally substituted 3-20 (e.g., 3-15, 3-10, 4-20, 5-20, 3, 4, 5, 6, 7, 8, 9, 10, 11, 12, 13, 14, 15, 16, 17, 18, 19, 20, etc.) membered heterocyclyl having 1-10 (e.g., 1-9, 1-8, 1-7, 1-6, 1-5, 1-4, 1, 2, 3, 4, 5, 6, 7, 8, 9, 10, etc.) heteroatoms. In some embodiments, R^{b} is optionally substituted 3-10 (e.g., 4-10, 5-10, 3, 4, 5, 6, 7, 8, 9, 10, etc.) membered heterocyclyl having 1-5 (e.g., 1-4, 1-3, 1, 2, 3, 4, 5, etc.) heteroatoms. In some embodiments, R^{b} is optionally substituted 3-7 membered heterocyclyl having 1 or 2 heteroatoms. In some embodiments, R^{b} is optionally substituted 3-7 membered heterocyclyl having one heteroatom. In some embodiments, R^{b} is optionally substituted tetrahydro-2H-pyranyl. In some embodiments, R^{b} is

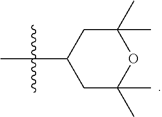 [SMILES: *C1CC(C)(C)OC(C)(C)C1]

In some embodiments, R is

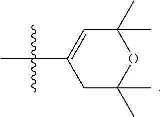 [SMILES: *C1=CC(C)(C)OC(C)(C)C1]

In some embodiments, R^{b} is

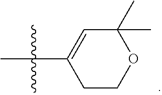 [SMILES: *C1=CC(C)(C)OCC1]

In some embodiments, R^{b} is

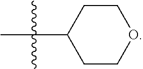 [SMILES: *C1CCOCC1]

In some embodiments, R^{b} is 4-tetrahydro-2H-pyranyl. In some embodiments, R^{b} is optionally substituted 3,6-dihydro-2H-pyranyl. In some embodiments, R^{b} is 3,6-dihydro-2H-pyran-4-yl. In some embodiments, R^{b} is optionally substituted

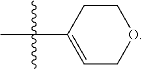 [SMILES: *C1=CCOCC1]

In some embodiments, R^{b} is

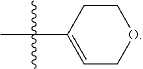 [SMILES: *C1=CCOCC1]

In some embodiments, R^{b} is optionally substituted azetidinyl. In some embodiments, R^{b} is azetidinyl optionally substituted with C₁₆ aliphatic (e.g., methyl, ethyl, n-propyl, s-propyl, or isopropyl) or halogen (e.g., —F, —Cl, —Br, or —I). In some embodiments, R^{b} is optionally substituted

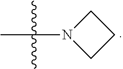 [SMILES: *N1CCC1]

In some embodiments, R^{b} is

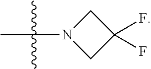 [SMILES: *N1CC(F)(F)C1]

In some embodiments, R^{b} is optionally substituted pyrrolidinyl. In some embodiments, R^{b} is pyrrolidinyl optionally substituted with C₁₋₆ aliphatic (e.g., methyl, ethyl, n-propyl, s-propyl, or isopropyl) or halogen (e.g., —F, —Cl, —Br, or —I). In some embodiments, R^{b} is optionally substituted

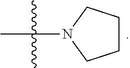 [SMILES: *N1CCCC1]

In some embodiments, R^{b} is

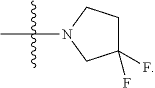 [SMILES: *N1CCC(F)(F)C1]

In some embodiments, R^{b} is optionally substituted piperidinyl. In some embodiments, R^{b} is piperidinyl optionally substituted with C₁₋₆ aliphatic (e.g., methyl, ethyl, n-propyl, s-propyl, or isopropyl) or halogen (e.g., —F, —Cl, —Br, or —I). In some embodiments, R is optionally substituted

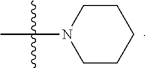 [SMILES: *N1CCCCC1]

In some embodiments, R^{b} is

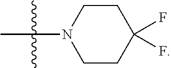 [SMILES: *N1CCC(F)(F)CC1]

In some embodiments, R^{b} is

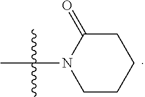 [SMILES: *N1CCCCC1=O]

In some embodiments, R^{b} is

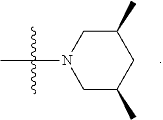 [SMILES: *N1C[C@H](C)C[C@H](C)C1]

In some embodiments, R^{b} is optionally substituted morpholinyl. In some embodiments, R^{b} is optionally substituted 4-morpholinyl. In some embodiments, R^{b} is

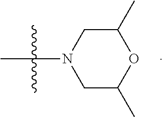 [SMILES: *N1CC(C)OC(C)C1]

In some embodiments, R^{b} is

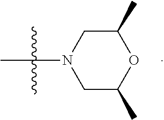 [SMILES: *N1C[C@@H](C)O[C@@H](C)C1]

In some embodiments, R^{b} is optionally substituted

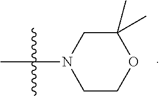 [SMILES: *N1CCOC(C)(C)C1]

In some embodiments, R^{b} is

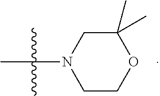 [SMILES: *N1CCOC(C)(C)C1]

In some embodiments, R^{b} is

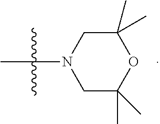 [SMILES: *N1CC(C)(C)OC(C)(C)C1]

In some embodiments, R^{b} is morpholinyl. In some embodiments, R^{b} is optionally substituted silinane. In some embodiments, R^{b} is

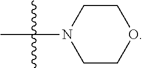 [SMILES: *N1CCOCC1]

In some embodiments, R^{b} is optionally substituted

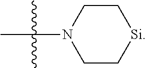

In some embodiments, R^{b} is

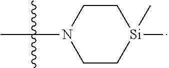

In some embodiments, R^{b} is optionally substituted

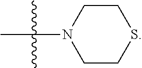 [SMILES: *N1CCSCC1]

In some embodiments, R^{b} is

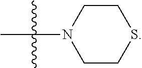 [SMILES: *N1CCSCC1]

In some embodiments, R^{b} is an optionally substituted 7-10 membered heterocyclyl having two heteroatoms. In some embodiments, R^{b} is optionally substituted 2-oxa-5-azabicyclo[2.2.1]heptanyl. In some embodiments, R^{b} is

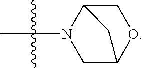 [SMILES: *N1CC2CC1CO2]

In some embodiments, R^{b} is optionally substituted 3-oxa-8-azabicyclo[3.2.1]octanyl. In some embodiments, R^{b} is

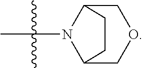 [SMILES: *N1C2CCC1COC2]

In some embodiments, R^{b} is an optionally substituted 7- to 12-membered heterospirocyclyl comprising 1-4 heteroatoms selected from nitrogen, oxygen, or sulfur. In some embodiments, R^{b} is an optionally substituted 7-membered heterospirocyclyl comprising 1 nitrogen heteroatom. In some embodiments, R^{b} is an optionally substituted

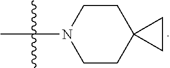 [SMILES: *N1CCC2(CC1)CC2]

In some embodiments, R^{b} is

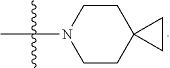 [SMILES: *N1CCC2(CC1)CC2]

In some embodiments, R^{b} is -L-R′, wherein R′ is R and R is as described herein. In some embodiments, R^{b} is —O—R′, wherein R′ is R and R is as described herein.

In some embodiments, R^{b} is —O—R′, wherein R′ is optionally substituted 3- to 8-membered saturated or partially unsaturated carbocyclyl. In some embodiments, R^{b} is —O—R′, wherein R′ is optionally substituted cyclohexyl. In some embodiments, R^{b} is —O—R′, wherein R′ is cyclohexyl optionally substituted with C₁₋₆ aliphatic (e.g., methyl, ethyl, n-propyl, s-propyl, or isopropyl) or halogen (e.g., —F, —Cl, —Br, or —I). In some embodiments, R^{b} is

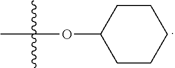 [SMILES: *OC1CCCCC1]

In some embodiments, R^{b} is —O—R′, wherein R′ is optionally substituted cyclooxtynyl. In some embodiments, R^{b} is

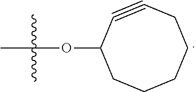 [SMILES: *OC1C#CCCCCC1]

In some embodiments, R^{b} is —O—R′, wherein R′ is optionally substituted phenyl. In some embodiments, R^{b} is —O—R′, wherein R′ is phenyl optionally substituted with C₁₋₆ aliphatic (e.g., methyl, ethyl, n-propyl, s-propyl, or isopropyl) or halogen (e.g., —F, —Cl, —Br, or —I). In some embodiments, R^{b} is

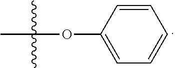 [SMILES: *Oc1ccccc1]

In some embodiments, R is

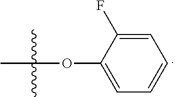 [SMILES: *Oc1ccccc1F]

In some embodiments, R^{b} is —O—R′, wherein R′ is an optionally substituted 3- to 7-membered heterocyclyl comprising 1-3 heteroatoms selected from nitrogen, oxygen, or sulfur. In some embodiments, R^{b} is —O—R′, wherein R′ is an optionally substituted tetrahydro-2H-pyranyl. In some embodiments, R^{b} is —O—R′, wherein R′ is tetrahydro-2H-pyranyl optionally substituted with C₁₋₆ aliphatic (e.g., methyl, ethyl, n-propyl, s-propyl, or isopropyl) or halogen (e.g., —F, —Cl, —Br, or —I). In some embodiments, R^{b} is

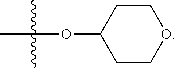 [SMILES: *OC1CCOCC1]

In some embodiments, R^{b} is or comprises a reactive group. Various reactive groups are known in the art, e.g., for amidation, esterification, cycloaddition, elimination, replacement, condensation, reduction, oxidation, coupling, etc., including various biocompatible reactions, and can be utilized in accordance with the present disclosure.

p

In some embodiments, p is 0. In some embodiments, p is 1. In some embodiments, p is 2. In some embodiments, p is 3. In some embodiments, p is 4. In some embodiments, p is 5. In some embodiments, p is 6. In some embodiments, p is 7. In some embodiments, p is 8. In some embodiments, p is 9. In some embodiments, p is 10.

In some embodiments, in a formula, e.g., formula A, A′, etc., p is 1. In some embodiments, R⁹ is at the para position relative to Y.

q

In some embodiments, q is 0. In some embodiments, q is 1. In some embodiments, q is 2. In some embodiments, q is 3. In some embodiments, q is 4. In some embodiments, q is 5. In some embodiments, q is 6. In some embodiments, q is 7. In some embodiments, q is 8. In some embodiments, q is 9. In some embodiments, q is 10.

In some embodiments, in a formula, e.g., formula A, A′, etc., q is 1. In some embodiments, R′ is at the para position relative to the other connection site of Ring A.

t

In some embodiments, t is 0. In some embodiments, t is 1. In some embodiments, t is 2. In some embodiments, t is 3. In some embodiments, t is 4. In some embodiments, t is 5. In some embodiments, t is 6. In some embodiments, t is 7. In some embodiments, t is 8. In some embodiments, t is 9.

In some embodiments, in a formula, e.g., formula A, A′, etc., t is 1. In some embodiments, R⁹ is at the para position relative to Y.

x

In some embodiments, x is 0. In some embodiments, x is 1. In some embodiments, x is 2. In some embodiments, x is 3. In some embodiments, x is 4.

y

In some embodiments, y is 0. In some embodiments, y is 1. In some embodiments, y is 2. In some embodiments, y is 3. In some embodiments, y is 4.

### L

In some embodiments, L is a covalent bond. In some embodiments, L is an optionally substituted bivalent C₁₋₁₀ (e.g., C₁₋₉, C₁₋₈, C₁₋₆, C₁, C₂, C₃, C₄, C₅, C₆, C₇, C₈, C₉, C₁₀, etc.) aliphatic wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—, and each R′ is independently as described herein. In some embodiments, L is an optionally substituted bivalent C₁₋₁₀ (e.g., C₁₋₉, C₁₋₈, C₁₋₆, C₁, C₂, C₃, C₄, C₅, C₆, C₇, C₈, C₉, C₁₀, etc.) heteroaliphatic having 1-6 (e.g., 1-5, 1-4, 1-3, 1, 2, 3, 4, 5, 6, etc.) heteroatoms wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—, and each R′ is independently as described herein. In some embodiments, L is optionally substituted bivalent C₁₋₁₀ (e.g., C₁₋₉, C₁₋₈, C₁₋₆, C₁, C₂, C₃, C₄, C₅, C₆, C₇, C₈, C₉, C₁₀, etc.) aliphatic. In some embodiments, L is optionally substituted bivalent linear C₁₋₁₀ (e.g., C₁₋₉, C₁₋₁₀, C₁₋₆, C₁, C₂, C₃, C₄, C₅, C₆, C₇, C₈, C₉, C₁₀, etc.) aliphatic. In some embodiments, L is optionally substituted bivalent branched C₁₋₁₀ (e.g., C₁₋₉, C₁₋₈, C₁₋₆, C₁, C₂, C₃, C₄, C₅, C₆, C₇, C₈, C₉, C₁₀, etc.) aliphatic. In some embodiments, one or more methylene units are independently as described herein. In some embodiments, L is or comprises —C(R′)₂— wherein each R′ is independently as described herein. In some embodiments, L is or comprises optionally substituted —CH₂—. In some embodiments, L is or comprises -Cy- as described herein. For example, in some embodiments, L is optionally substituted 3-10 membered cycloalkylene. In some embodiments, L is or comprises —O—. In some embodiments, L is or comprises —S—. In some embodiments, L is or comprises —N(R′)— wherein R′ is as described herein. In some embodiments, L is or comprises —N(R′)— wherein R′ is optionally substituted C₁₋₆ aliphatic. In some embodiments, L is or comprises —NH—. In some embodiments, L is or comprises —C(O)—. In some embodiments, L is or comprises -Cy-C(O)O—. In some embodiments, L is -Cy-C(O)O—, wherein -Cy- is optionally substituted 3-10 membered cycloalkylene. In some embodiments, L is or comprises —C(S)—. In some embodiments, L is or comprises —C(NR′)— wherein R′ is as described herein. In some embodiments, L is or comprise —C(NH)—. In some embodiments, L is or comprises —C(O)N(R′)— wherein R′ is as described herein. In some embodiments, L is or comprises -Cy-C(O)N(R′)— wherein each of -Cy- and R′ is independently as described herein (for example, in some embodiments, R′ is —H; in some embodiments, R′ is optionally substituted C₁₋₆ aliphatic). In some embodiments, L is or comprises —C(O)NH—. In some embodiments, L is or comprises —C(S)N(R′) wherein R′ is as described herein -. In some embodiments, L is or comprises —C(S)NH—. In some embodiments, L is or comprises —C(NR′)N(R′)— wherein each R′ is independently as described herein. In some embodiments, L is or comprises —C(NH)NH—. In some embodiments, L is or comprises —N(R′)C(O)N(R′)— wherein each R′ is independently as described herein. In some embodiments, L is or comprises —NHC(O)NH—. In some embodiments, L is or comprises —N(R′)C(O)O— wherein R′ is as described herein. In some embodiments, L is or comprises —NHC(O)O—. In some embodiments, L is or comprises —S(O)—. In some embodiments, L is or comprises —S(O)₂—. In some embodiments, L is or comprises —S(O)₂N(R′)— wherein R′ is as described herein. In some embodiments, L is or comprises —S(O)₂NH—. In some embodiments, L is or comprises —C(O)S—. In some embodiments, L is or comprises —C(O)O—.

In some embodiments, L is -L″-L^{x}-L^{y}-L^{z}-, wherein L″ is a covalent bond or an optionally substituted bivalent C₁₋₇ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—; and each of U, L and L^{Z} is independently a covalent bond or optionally substituted methylene which is optionally replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—. In some embodiments, U is a covalent bond, —N(R′), or —O—. In some embodiments, L^{y} is a covalent bond, —C(O)—, —C(S)—, —C(NR′)—, —C(O)N(R′)—, —C(S)N(R′)—, or —C(NR′)(NR′)—. In some embodiments, L^{z} is a covalent bond, —N(R′)—, —N(R′)O— or —O—. In some embodiments, L^{x} is a covalent bond. In some embodiments, L^{x} is —N(R′)— wherein R′ is as described herein. In some embodiments, LX is —O—. In some embodiments, L is a covalent bond. In some embodiments, L is —C(O)—. In some embodiments, L^{y} is —C(S)—. In some embodiments, L is —C(NR′)— wherein R′ is as described herein. In some embodiments, L^{y} is —C(O)N(R′)— wherein R′ is as described herein. In some embodiments, L is —C(S)N(R′)— wherein R′ is as described herein. In some embodiments, L is —C(NR′)(NR′)— wherein each R′ is independently as described herein. In some embodiments, U is a covalent bond. In some embodiments, L^{z} is —N(R′)— wherein R′ is as described herein. In some embodiments, L^{z} is —N(R′)O— wherein R′ is as described herein. In some embodiments, L^{z} is —O—.

### Cy

As described herein, -Cy- is optionally substituted (in addition to the two group it is bonded to). In some embodiments, -Cy- is substituted. In some embodiments, -Cy- is unsubstituted.

In some embodiments, -Cy- is an optionally substituted ring as described herein. In some embodiments, -Cy- is 3-20, 3-15, 3-10, 3-8, 3-6, 5-6, or 3, 4, 5, 6, 7, 8, 9, 10, 11, 12, 13, 14, 15, 16, 17, 18, 19, or 20, etc. membered. In some embodiments, -Cy- is 3-membered. In some embodiments, -Cy- is 4-membered. In some embodiments, -Cy- is 5-membered. In some embodiments, -Cy- is 6-membered. In some embodiments, -Cy- is 7-membered. In some embodiments, -Cy- is 8-membered. In some embodiments, -Cy- is 9-membered. In some embodiments, -Cy- is 10-membered. In some embodiments, -Cy- is 11-membered. In some embodiments, -Cy- is 12-membered. In some embodiments, -Cy- is saturated. In some embodiments, -Cy- is partially unsaturated. In some embodiments, -Cy- is aromatic. In some embodiments, -Cy- is monocyclic. In some embodiments, it is bicyclic. In some embodiments, it is polycyclic. In some embodiments, each monocyclic unit is independently a 3-15 (e.g., 3-15, 3-10, 3-8, 3-6, 5-6, or 3, 4, 5, 6, 7, 8, 9, 10, 11, 12, 13, 14, or 15, etc.) membered ring which is independently saturated, partially unsaturated or aromatic and has 0-4 heteroatoms. In some embodiments, each monocyclic unit is independently a 3-10 (e.g., 3-10, 3-8, 3-6, 5-6, or 3, 4, 5, 6, 7, 8, 9, or 10, etc.) membered ring which is independently saturated, partially unsaturated or aromatic and has 0-4 (e.g., 0, 1, 2, 3, or 4, etc.) heteroatoms independently selected from nitrogen, oxygen and sulfur. In some embodiments, each monocyclic ring unit is independently 3-7 membered. In some embodiments, each monocyclic ring unit is independently 3-6 membered. In some embodiments, each monocyclic ring unit is independently 5-7 membered. In some embodiments, each monocyclic unit is independently saturated or partially unsaturated. In some embodiments, at least one monocyclic unit is saturated. In some embodiments, at least one monocyclic unit is partially unsaturated. In some embodiments, at least one monocyclic unit is aromatic. In some embodiments, -Cy- has 1-10 (e.g., 1, 2, 3, 4, 5, 6, 7, 8, 9, or 10, etc.) heteroatoms independently selected from oxygen, nitrogen, sulfur, phosphorus and silicon. In some embodiments, -Cy- has 1-5 (e.g., 1, 2, 3, 4, or 5, etc.) heteroatoms independently selected from oxygen, nitrogen, sulfur, phosphorus and silicon. In some embodiments, there are no additional heteroatoms. In some embodiments, there is one additional heteroatom. In some embodiments, there are 2 additional heteroatoms. In some embodiments, there are 3 additional heteroatoms. In some embodiments, there are 4 additional heteroatoms. In some embodiments, there are 5 additional heteroatoms. In some embodiments, there are 6 or more additional heteroatoms. In some embodiments, an additional heteroatom is nitrogen. In some embodiments, an additional heteroatom is oxygen. In some embodiments, an additional heteroatom is sulfur.

In some embodiments, -Cy- is an optionally substituted 5-10 membered aromatic ring having 0-5 heteroatoms independently selected from oxygen, nitrogen, and sulfur. In some embodiments, -Cy- is an optionally substituted 5-6 membered aromatic ring having 0-5 heteroatoms independently selected from oxygen, nitrogen, and sulfur. In some embodiments, -Cy- is an optionally substituted phenyl ring. In some embodiments, -Cy- is a phenyl ring. In some embodiments, -Cy- is an optionally substituted 10-membered bicyclic aryl ring. In some embodiments, -Cy- is an optionally substituted 5-9 membered heteroaryl ring having 1-4 heteroatoms independently selected from nitrogen, oxygen and sulfur. In some embodiments, -Cy- is an optionally substituted 5-membered heteroaryl ring having 1-4 heteroatoms independently selected from nitrogen, oxygen and sulfur. In some embodiments, -Cy- is an optionally substituted 6-membered heteroaryl ring having 1-4 heteroatoms independently selected from nitrogen, oxygen and sulfur. In some embodiments, -Cy- is an optionally substituted 9-membered bicyclic heteroaryl ring having 1-4 heteroatoms independently selected from nitrogen, oxygen and sulfur. In some embodiments, a heteroatom is nitrogen.

In some embodiments, -Cy- is an optionally substituted bicyclic ring, wherein at least one monocyclic ring is an optionally substituted phenyl ring. In some embodiments, -Cy- is an optionally substituted bicyclic ring, wherein at least one monocyclic ring is an optionally substituted 5-6 membered heteroaryl ring having 1-3 (e.g., 1-2, 1, 2, 3, etc.) heteroatoms. In some embodiments, -Cy- is an optionally substituted bicyclic ring, wherein each monocyclic ring is independently an optionally substituted phenyl or 5-6 membered heteroaryl ring having 1-3 (e.g., 1-2, 1, 2, 3, etc.) heteroatoms. In some embodiments, -Cy- is an optionally substituted bicyclic ring, wherein one monocyclic ring is an optionally substituted phenyl or 5-6 membered heteroaryl ring having 1-3 (e.g., 1-2, 1, 2, 3, etc.) heteroatoms, and the other monocyclic ring is an optionally substituted non-aromatic 3-10 (e.g., 3-9, 4-10, 5-10, 3, 4, 5, 6, 7, 8, 9, 10, etc.) membered ring having 1-4 (e.g., 1-2, 1-3, 1, 2, 3, 4, etc.) heteroatoms. In some embodiments, -Cy- is an optionally substituted bicyclic ring, wherein one monocyclic ring is an optionally substituted phenyl or 5-6 membered heteroaryl ring having 1-3 heteroatoms, and the other monocyclic ring is an optionally substituted non-aromatic 5-6 membered ring having 1-3 (e.g., 1-2, 1, 2, 3, etc.) heteroatoms. In some embodiments, -Cy- is an optionally substituted 3-10 (e.g., 3-9, 5-10, 3, 4, 5, 6, 7, 8, 9, 10, etc.) membered bivalent cycloaliphatic ring. In some embodiments, -Cy- is an optionally substituted 6-membered cycloalkyl ring. In some embodiments, -Cy- is optionally substituted phenylene. In some embodiments, -Cy- is optionally substituted 1, 4-phenylene. In some embodiments, -Cy- is 1, 4-phenylene. In some embodiments, -Cy- is optionally substituted bivalent cyclohexyl. In some embodiments, -Cy- is optionally substituted 1, 4-cyclohexyl. In some embodiments, -Cy- is 1, 4-cyclohexyl. In some embodiments, -Cy- is an optionally substituted bivalent piperidyl ring. In some embodiments, -Cy- is a bivalent piperidyl ring. In some embodiments, a bivalent piperidyl ring is bivalent 1,2-piperidyl. In some embodiments, a bivalent piperidyl ring is bivalent 1,4-piperidyl. In some embodiments, -Cy- is an optionally substituted bivalent piperazinyl ring. In some embodiments, -Cy- is a bivalent piperazinyl ring. In some embodiments, a bivalent piperidyl ring is bivalent 1,2-piperazinyl. In some embodiments, a bivalent piperidyl ring is bivalent 1,4-piperazinyl. In some embodiments, -Cy- is an optionally substituted 5-6 membered bivalent heteroaryl ring. In some embodiments, -Cy- is an optionally substituted 5-membered bivalent heteroaryl ring. In some embodiments, -Cy- is an optionally substituted 6-membered bivalent heteroaryl ring having 1 or 3 nitrogen atoms. In some embodiments, -Cy- is an optionally substituted 6-membered bivalent heteroaryl ring having a nitrogen atom. In some embodiments, -Cy- is an optionally substituted bivalent pyridinyl ring. In some embodiments, -Cy- is a bivalent pyridinyl ring. In some embodiments, a bivalent pyridinyl ring is a 2, 5-bivalent pyridinyl ring. In some embodiments, a bivalent pyridinyl ring is a 3, 5-bivalent pyridinyl ring. In some embodiments, -Cy- is a bivalent pyrimidinyl ring. In some embodiments, a bivalent pyrimidinyl ring is a 2, 5-bivalent pyrimidinyl ring. In some embodiments, -Cy- is a bivalent thiazolyl ring. In some embodiments, a bivalent thiazolyl ring is a 2, 5-bivalent thiazolyl ring.

### R″

In some embodiments, R″ is —H.

In some embodiments, R″ is halogen. In some embodiments, R″ is —F. In some embodiments, R″ is —Cl. In some embodiments, R″ is —Br. In some embodiments, R″ is —I.

In some embodiments, R″ is -L-R′ wherein each variable is independently as described herein. In some embodiments, R″ is R′ as described herein. In some embodiments, R″ is -L-R wherein each variable is independently as described herein. In some embodiments, R″ is R as described herein.

In some embodiments, R″ is -L-OR′. In some embodiments, R″ is -L-SR′. In some embodiments, R″ is -L-C(O)OR′. In some embodiments, R″ is -L-C(O)SR′. In some embodiments, R″ is -L-C(O)N(R′)₂. In some embodiments, R″ is -L-OC(O)N(R′)₂. In some embodiments, R″ is -L-C(O)R′. In some embodiments, R″ is -L-N(R′)₂. In some embodiments, R″ is —CN. In some embodiments, R″ is —OC(R′)₂COOH. In some embodiments, R″ is —SC(R′)₂COOH. In some embodiments, R″ is —N(R′)C(R′)₂COOH. In some embodiments, R″ is optionally substituted alkylsulfonyl. In some embodiments, R″ is arylsulfonyl. In some embodiments, R″ is carboxylate. In some embodiments, R″ is ester. In some embodiments, R″ is ether. In some embodiments, R″ is amide. In some embodiments, R″ is carbohydrate. In some embodiments, R″ is amino acid. In some embodiments, R″ is acyl. In some embodiments, R″ is alkyloxy-substituted acyl. In some embodiments, R″ is alditol. In some embodiments, R″ is sulfate. In some embodiments, R″ is sulfonamide. In some embodiments, R″ is sulfoxide. In some embodiments, R″ is sulfonate. In some embodiments, R″ is sulfone. In some embodiments, R″ is thioalkyl. In some embodiments, R″ is thioester. In some embodiments, R″ is thioether. In some embodiments, R″ is or comprise a peptide moiety. In some embodiments, R″ is or comprises a carbohydrate moiety.

### R′

In some embodiments, R′ is R as described herein. In some embodiments, R′ is —H. In some embodiments, R′ is —OR wherein R is as described herein. In some embodiments, R′ is —C(O)R wherein R is as described herein. In some embodiments, R′ is —C(O)R wherein R is as described herein. In some embodiments, R′ is —C(O)OR wherein R is as described herein. In some embodiments, R′ is —C(O)N(R)₂, wherein each R is independently as described herein. In some embodiments, R′ is —S(O)R wherein R is as described herein. In some embodiments, R′ is —S(O)₂R wherein R is as described herein. In some embodiments, R′ is —S(O)₂CH₃. In some embodiments, R′ is —C(O)CH₃. In some embodiments, R′ is —C(O)R wherein R is optionally substituted C₆₋₁₄ aryl. In some embodiments, R′ is —C(O)R wherein R is optionally substituted phenyl. In some embodiments, R′ is —C(O)R wherein R is 3-iodophenyl. In some embodiments, R′ is —C(O)OR wherein R is as described herein. In some embodiments, R′ is —C(O)OH. In some embodiments, R′ is optionally substituted C₁₋₁₀ aliphatic. In some embodiments, R′ is optionally substituted C₁₋₆ aliphatic. In some embodiments, R′ is optionally substituted C₁₋₆ alkyl. In some embodiments, R′ is methyl.

### R

Various embodiments for R are extensively described herein, including in various sections for other variables that can be R (e.g., R^{s}, R¹, R², R′, etc.).

In some embodiments, R is —H. In some embodiments, R is not-H.

In some embodiments, each R is independently hydrogen, or an optionally substituted group selected from C₁₋₂₀ (e.g., C₁₋₁₅, C₁₋₁₀, C₁₋₉, C₁₋₈, C₁₋₆, C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, etc.) aliphatic, C₁₋₂₀ (e.g., C₁₋₁₅, C₁₋₁₀, C₁₋₉, C₁₋₈, C₁₋₆, C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, etc.) heteroaliphatic having 1-10 (e.g., 1-9, 1-8, 1-7, 1-6, 1-5, 1-4, 1, 2, 3, 4, 5, 6, 7, 8, 9, 10, etc.) heteroatoms, C₃₋₂₀ (e.g., 3-15, 3-10, 4-20, 5-20, 3, 4, 5, 6, 7, 8, 9, 10, 11, 12, 13, 14, 15, 16, 17, 18, 19, 20, etc. membered) heterocyclyl having 1-10 (e.g., 1-9, 1-8, 1-7, 1-6, 1-5, 1-4, 1, 2, 3, 4, 5, 6, 7, 8, 9, 10, etc.) heteroatoms, C₅₋₂₀ (e.g., 5-15, 5-14, 5-10, 5, 6, 7, 8, 9, 10, 11, 12, 13, 14, 15, 16, 17, 18, 19, 20, etc. membered) aryl, and 5-20 (e.g., 5-15, 5-14, 5-10, 5, 6, 7, 8, 9, 10, 11, 12, 13, 14, 15, 16, 17, 18, 19, 20, etc.) membered heteroaryl having 1-10 (e.g., 1-9, 1-8, 1-7, 1-6, 1-5, 1-4, 1, 2, 3, 4, 5, 6, 7, 8, 9, 10, etc.) heteroatoms.

In some embodiments, R is optionally substituted C₁₋₂₀ (e.g., C₁₈1₅, C₁₋₁₀, C₁₋₉, C₁₋₈, C₁₋₆, C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, etc.) aliphatic. In some embodiments, R is optionally substituted C₁₋₁₀ aliphatic. In some embodiments, an aliphatic group is an alkyl group. In some embodiments, R is C₁₋₆ aliphatic. In some embodiments, R is C₁₋₆ alkyl. In some embodiments, R is optionally substituted methyl. In some embodiments, R is optionally substituted ethyl. In some embodiments, R is optionally substituted n-propyl. In some embodiments, R is optionally substituted isopropyl. In some embodiments, R is n-butyl. In some embodiments, R is t-butyl. In some embodiments, R is pentyl. In some embodiments, R is hexyl.

In some embodiments, an aliphatic group is or comprises a cycloaliphatic ring. In some embodiments, R is optionally substituted C₃₋₁₅ (e.g., C₃₋₁₅, C₃₋₁₂, C₃₋₁₀, C₄₋₁₀, C₃₋₉, C₃₋₇, or 3, 4, 5, 6, 7, 8, 9, 10, 11, 12, 13, 14, or 15 etc.) cycloaliphatic. In some embodiments, R is optionally substituted C₃₋₁₀ cycloaliphatic. In some embodiments, an aliphatic group is a cycloalkyl group. In some embodiments, a cycloaliphatic group is monocyclic. In some embodiments, it is bicyclic. In some embodiments, it is polycyclic. In some embodiments, each monocyclic unit is independently a 3-10 (e.g., C₄₋₁₀, C₃₋₉, C₃₋₇, or 3, 4, 5, 6, 7, 8, 9, or 10, etc.) membered cycloaliphatic ring. In some embodiments, a cycloaliphatic group is saturated. In some embodiments, it is partially unsaturated. In some embodiments, R is optionally substituted cyclopropyl. In some embodiments, R is optionally substituted cyclobutyl. In some embodiments, R is optionally substituted cyclopentyl. In some embodiments, R is optionally substituted cyclohexyl. In some embodiments, R is optionally substituted cycloheptyl.

In some embodiments, R is optionally substituted C₁₋₂₀ (e.g., C₁₋₁₅, C₁₋₁₀, C₁₋₉, C₁₋₈, C₁₋₆, C₁, C₂, C₃, C₄, C₅, C₆, C₇, C₈, C₉, C₁₀, C₁₁, C₁₂, C₁₃, C₁₄, C₁₅, C₁₆, C₁₇, Cis, C₁₉, C₂₀, etc.) having 1-10 (e.g., 1-5, 1, 2, 3, 4, 5, 6, 7, 8, 9, 10, etc.) heteroatoms. In some embodiments, R is optionally substituted C₁₋₁₅ (e.g., C₁₋₁₅, C₁₋₁₂, C₁₋₁₀, etc.) heteroaliphatic having 1-5 (e.g., 1, 2, 3, 4, or 5) heteroatoms independently selected from oxygen, nitrogen, sulfur, phosphorus and silicon. In some embodiments, R is optionally substituted C₁₋₁₅ (e.g., C₁₋₁₅, C₁₋₁₂, C₁₋₁₀, etc.) heteroaliphatic having 1-5 (e.g., 1, 2, 3, 4, or 5) heteroatoms independently selected from nitrogen, oxygen and sulfur. In some embodiments, R is C₁₋₁₀ heteroaliphatic having 1-5 (e.g., 1, 2, 3, 4, or 5, etc.) heteroatoms independently selected from nitrogen, oxygen and sulfur. In some embodiments, R is C₁₋₁₀ heteroaliphatic having 1 or 2 heteroatoms independently selected from nitrogen, oxygen and sulfur. In some embodiments, R is C₁₋₁₀ heteroaliphatic having one heteroatom selected from nitrogen, oxygen and sulfur. In some embodiments, a heteroatom is nitrogen. In some embodiments, a heteroatom is oxygen. In some embodiments, a heteroatom is sulfur.

In some embodiments, R is optionally substituted C₆₋₂₀ (e.g., C₆₋₁₄, C₆₋₁₀, C₆, C₁₀, C₁₄, etc.) aryl. In some embodiments, R is optionally substituted C₆₋₁₄ (e.g., C₆₋₁₄, C₆₋₁₀, C₆₋₉, etc.) aryl. In some embodiments, R is optionally substituted C₆₋₁₀ aryl. In some embodiments, R is optionally substituted 6-, 10-, or 14-membered hydrocarbon aryl. In some embodiments, an aryl ring is monocyclic. In some embodiments, an aryl ring is bicyclic. In some embodiments, an aryl ring is polycyclic. In some embodiments, each monocyclic unit is independently a 6-membered aromatic ring. In some embodiments, R is optionally substituted phenyl. In some embodiments, R is phenyl. In some embodiments, R is optionally substituted 10-membered aryl. In some embodiments, R is optionally substituted naphthyl. In some embodiments, R is naphthyl.

In some embodiments, R is 5-20 (e.g., 5-14, 5-10, 5-9, 5, 6, 7, 8, 9, 10, 11, 12, 13, 14, 15, 16, 17, 18, 19, 20, etc.) membered heteroaryl having 1-10 (e.g., 1-9, 1-8, 1-6, 1-5, 1-4, 1, 2, 3, 4, 5, 6, 7, 8, 9, 10 etc.) heteroatoms. In some embodiments, R is optionally substituted 5-14 (e.g., 5-10, 5-9, 5, 6, 7, 8, 9, 10, 11, 12, 13, 14, etc.) membered heteroaryl having 1-5 (e.g., 1-5, 1-4, 1, 2, 3, 4, 5 etc.) heteroatoms independently selected from oxygen, nitrogen, sulfur, phosphorus and silicon. In some embodiments, R is optionally substituted 5-14 (e.g., 5-10, 5-9, 5, 6, 7, 8, 9, 10, 11, 12, 13, 14, etc.) membered heteroaryl having 1-5 (e.g., 1-5, 1-4, 1, 2, 3, 4, 5 etc.) heteroatoms independently selected from nitrogen, oxygen and sulfur. In some embodiments, R is 5-10 (e.g., 5-9, 5, 6, 9, 10 etc.) membered heteroaryl having 1-4 (e.g., 1-3, 1-2, 1, 2, 3, 4, etc.) heteroatoms independently selected from nitrogen, oxygen and sulfur. In some embodiments, a heteroaryl ring is monocyclic. In some embodiments, a heteroaryl ring is bicyclic. In some embodiments, a heteroaryl ring is polycyclic. In some embodiments, each monocyclic unit is independently a 5- or 6-membered aromatic ring having 0-4 heteroatoms, e.g., independently selected from nitrogen, oxygen and sulfur, wherein at least one monocyclic unit contains 1-4 heteroatoms. In some embodiments, R is optionally substituted 5-membered monocyclic heteroaryl having 1-4 heteroatoms independently selected from nitrogen, oxygen and sulfur. In some embodiments, R is optionally substituted 6-membered monocyclic heteroaryl having 1-4 heteroatoms independently selected from nitrogen, oxygen and sulfur. In some embodiments, R is optionally substituted 9-membered bicyclic heteroaryl having 1-4 heteroatoms independently selected from nitrogen, oxygen and sulfur. In some embodiments, R is optionally substituted 10-membered bicyclic heteroaryl having 1-4 heteroatoms independently selected from nitrogen, oxygen and sulfur. In some embodiments, a heteroaryl ring has one heteroatom. In some embodiments, a heteroaryl ring has two or more heteroatoms. In some embodiments, a heteroaryl ring has three or more heteroatoms. In some embodiments, a heteroaryl ring has four or more heteroatoms. In some embodiments, a heteroatom is nitrogen. In some embodiments, a heteroatom is oxygen. In some embodiments, a heteroatom is sulfur.

In some embodiments, R is optionally substituted 5-14 membered heteroaryl having 1-5 heteroatoms-C₁₋₁₅ aliphatic wherein the heteroaryl and aliphatic are independently as described herein. In some embodiments, R is optionally substituted 5-14 membered heteroaryl having 1-5 heteroatoms-C₁.is aliphatic. In some embodiments, R is optionally substituted 5-10 membered heteroaryl having 1-5 heteroatoms-C₁₋₁₅ aliphatic. In some embodiments, R is optionally substituted 5-10 membered heteroaryl having 1-5 heteroatoms-C₁₋₁₀ aliphatic. Various suitable heteroaryl and aliphatic groups are as described herein.

In some embodiments, R is optionally substituted 3-20 (e.g., 3-15, 3-10, 3, 4, 5, 6, 7, 8, 9, 10, 11, 12, 13, 14, 15, 16, 17, 18, 19, 20, etc.) membered heterocyclyl having 1-10 (e.g., 1-6, 1-3, 1-2, 1, 2, 3, 4, 5, 6, 7, 8, 9, 10, etc.) heteroatoms. In some embodiments, R is 3-20 (e.g., 3-15, 3-10, 3, 4, 5, 6, 7, 8, 9, 10, 11, 12, 13, 14, 15, 16, 17, 18, 19, 20, etc.) membered heterocyclyl having 1-10 (e.g., 1-5, 1-3, 1, 2, 3, 4, 5, 6, 7, 8, 9, 10, etc.) heteroatoms independently selected from nitrogen, oxygen and sulfur. In some embodiments, R is 3-20 (e.g., 3-20, 3-15, 3-10, 3, 4, 5, 6, 7, 8, 9, 10, 11, 12, 13, 14, 15, 16, 17, 18, 19, 20, etc.) membered heterocyclyl having 1-5 (e.g., 1-4, 1-3, 1, 2, 3, 4, 5, etc.) heteroatoms independently selected from nitrogen, oxygen and sulfur. In some embodiments, R is 3-10 (e.g., 3-9, 3-8, 3-7, 3, 4, 5, 6, 7, 8, 9, 10, etc.) membered heterocyclyl having 1-5 (e.g., 1-4, 1-3, 1-2, 1, 2, 3, 4, 5, etc.) heteroatoms. In some embodiments, a heterocyclyl group is monocyclic. In some embodiments, it is bicyclic. In some embodiments, it is polycyclic. In some embodiments, each monocyclic unit is independently a 3-10 (e.g., C₄₋₁₀, C₃₋₉, C₃₋₇, or 3, 4, 5, 6, 7, 8, 9, or 10, etc.) membered heterocyclyl ring having 1-5 (e.g., 1, 2, 3, 4, or 5, etc.) heteroatoms independently selected from nitrogen, oxygen and sulfur. In some embodiments, a heterocyclyl group is saturated. In some embodiments, it is partially unsaturated. In some embodiments, a heterocyclyl ring has one heteroatom. In some embodiments, a heterocyclyl ring has two or more heteroatoms. In some embodiments, a heterocyclyl ring has three or more heteroatoms. In some embodiments, a heterocyclyl ring has four or more heteroatoms. In some embodiments, a heteroatom is nitrogen. In some embodiments, a heteroatom is oxygen. In some embodiments, a heteroatom is sulfur. In some embodiments, each heteroatom is independently selected from oxygen, nitrogen, sulfur, phosphorus and silicon. In some embodiments, each heteroatom is independently selected from oxygen, nitrogen, and sulfur.

In some embodiments, R is an optionally substituted combination of two or more of C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms. For example, in some embodiments, R is optionally substituted C₆₋₂₀ aryl-C₁₋₂₀ aliphatic, C₆₋₂₀ aryl-C₁₋₂₀ heteroaliphatic having 1-10 heteroatoms, C₁₋₂₀ aliphatic-C₆₋₂₀ aryl, C₁₋₂₀ heteroaliphatic having 1-10 heteroatoms-C₆₋₂₀ aryl, 5-20 membered heteroaryl having 1-10 heteroatoms-C-20 heteroaliphatic having 1-10 heteroatoms, C₁₋₂₀ aliphatic-5-20 membered heteroaryl having 1-10 heteroatoms, C₁₋₂₀ heteroaliphatic having 1-10 heteroatoms-5-20 membered heteroaryl having 1-10 heteroatoms, etc. In some embodiments, there are 1-30 (e.g., 1-20, 2-10, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, etc.) carbon atoms and 0-10 (e.g., 1-10, 1-9, 1-8, 1-7, 1-6, 1-5, 1-4, 0, 1, 2, 3, 4, 5, 6, 7, 8, 9, 10, etc.) heteroatoms.

In some embodiments, two R groups are optionally and independently taken together to form a covalent bond. In some embodiments, two R groups attached to neighboring atoms are optionally and independently taken together to form a covalent bond. In some embodiments, two or more R groups are taken together with their intervening atom(s) to form an optionally substituted ring as described herein.

In some embodiments, two R groups are optionally and independently taken together with their intervening atom(s) to form an optionally substituted, 3-20 (e.g., 3-15, 3-10, 3, 4, 5, 6, 7, 8, 9, 10, 11, 12, 13, 14, 15, 16, 17, 18, 19, 20, etc.) membered, monocyclic, bicyclic or polycyclic ring having, in addition to the intervening atom(s), 0-10 (e.g., 1-10, 1-5, 0, 1, 2, 3, 4, 5, 6, 7, 8, 9 or 10, etc.) heteroatoms. In some embodiments, two or more R groups are optionally and independently taken together with their intervening atoms to form an optionally substituted, 3-20 (e.g., 3-15, 3-10, 3, 4, 5, 6, 7, 8, 9, 10, 11, 12, 13, 14, 15, 16, 17, 18, 19, 20, etc.) membered, monocyclic, bicyclic or polycyclic ring having, in addition to the intervening atom(s), 0-10 (e.g., 1-10, 1-5, 0, 1, 2, 3, 4, 5, 6, 7, 8, 9 or 10, etc.) heteroatoms.

As described herein, in various instances, two or more R groups, or two or more groups that are or can be R (e.g., R^{s}, R′, etc.), can be together with their intervening atom(s) to form an optionally substituted ring as described herein. In some embodiments, a formed ring is substituted (in addition to groups attached to the intervening atom(s). In some embodiments, a formed ring is unsubstituted. In some embodiments, a formed ring is 3-20, 3-15, 3-10, 3-8, 3-6, 5-6, 3, 4, 5, 6, 7, 8, 9, 10, 11, 12, 13, 14, 15, 16, 17, 18, 19, 20, etc. membered. In some embodiments, a formed ring is 3-membered. In some embodiments, a formed ring is 4-membered. In some embodiments, a formed ring is 5-membered. In some embodiments, a formed ring is 6-membered. In some embodiments, a formed ring is 7-membered. In some embodiments, a formed ring is 8-membered. In some embodiments, a formed ring is 9-membered. In some embodiments, a formed ring is 10-membered. In some embodiments, a formed ring is 11-membered. In some embodiments, a formed ring is 12-membered. In some embodiments, a formed ring is saturated. In some embodiments, a formed ring is partially unsaturated. In some embodiments, a formed ring is aromatic. In some embodiments, a formed ring is monocyclic. In some embodiments, it is bicyclic. In some embodiments, it is polycyclic. In some embodiments, each monocyclic unit is independently a 3-15 (e.g., 3-15, 3-10, 3-8, 3-6, 5-6, or 3, 4, 5, 6, 7, 8, 9, 10, 11, 12, 13, 14, or 15, etc.) membered ring which is independently saturated, partially unsaturated or aromatic and has 0-4 heteroatoms. In some embodiments, each monocyclic unit is independently a 3-10 (e.g., 3-10, 3-8, 3-6, 5-6, or 3, 4, 5, 6, 7, 8, 9, or 10, etc.) membered ring which is independently saturated, partially unsaturated or aromatic and has 0-4 (e.g., 0, 1, 2, 3, or 4, etc.) heteroatoms independently selected from nitrogen, oxygen and sulfur. In some embodiments, each monocyclic ring unit is independently 3-7 membered. In some embodiments, each monocyclic ring unit is independently 3-6 membered. In some embodiments, each monocyclic ring unit is independently 5-7 membered. In some embodiments, each monocyclic unit is independently saturated or partially unsaturated. In some embodiments, at least one monocyclic unit is saturated. In some embodiments, at least one monocyclic unit is partially unsaturated. In some embodiments, at least one monocyclic unit is aromatic. In some embodiments, a formed ring has, in addition to the intervening atom(s), 0-10 (e.g., 0, 1, 2, 3, 4, 5, 6, 7, 8, 9, or 10, etc.) heteroatoms independently selected from oxygen, nitrogen, sulfur, phosphorus and silicon. In some embodiments, a formed ring has, in addition to the intervening atom(s), 0-5 (e.g., 0, 1, 2, 3, 4, or 5, etc.) heteroatoms independently selected from oxygen, nitrogen, sulfur, phosphorus and silicon. In some embodiments, there are no additional heteroatoms. In some embodiments, there is one additional heteroatom. In some embodiments, there are 2 additional heteroatoms. In some embodiments, there are 3 additional heteroatoms. In some embodiments, there are 4 additional heteroatoms. In some embodiments, there are 5 additional heteroatoms. In some embodiments, there are 6 or more additional heteroatoms. In some embodiments, an additional heteroatom is nitrogen. In some embodiments, an additional heteroatom is oxygen. In some embodiments, an additional heteroatom is sulfur.

In some embodiments, each R is independently —H, or an optionally substituted group selected from C₁₋₁₀ aliphatic, C₁₋₁₀ heteroaliphatic having 1-5 heteroatoms, C₆₋₁₄ aryl, 5-14 membered heteroaryl having 1-10 heteroatoms, and 3-10 membered heterocyclyl having 1-5 heteroatoms, or
two R groups are optionally and independently taken together to form a covalent bond, or:
two or more R groups on the same atom are optionally and independently taken together with the atom to form an optionally substituted, 3-15 (e.g., 3-12, 3-10, 3-8, 4-6, 5-6, 3, 4, 5, 6, 7, 8, 9, 10, 11, 12, 13, 14 15, etc.) membered, monocyclic, bicyclic or polycyclic ring having, in addition to the atom, 0-5 (e.g., 1-5, 0, 1, 2, 3, 4, 5) heteroatoms; or
two or more R groups on two or more atoms are optionally and independently taken together with their intervening atoms to form an optionally substituted, 3-15 (e.g., 3-12, 3-10, 3-8, 4-6, 5-6, 3, 4, 5, 6, 7, 8, 9, 10, 11, 12, 13, 14, 15, etc.) membered, monocyclic, bicyclic or polycyclic ring having, in addition to the intervening atoms, 0-5 (e.g., 1-5, 0, 1, 2, 3, 4, 5, etc.) heteroatoms.

In some embodiments, as used in the present disclosure, e.g., in various embodiments or R, R′, R^{s}, R′, various formulae, etc., each heteroatom is independently selected from oxygen, nitrogen, sulfur, phosphorus and silicon. In some embodiments, each heteroatom is independently selected from oxygen, nitrogen and sulfur. In some embodiments, in provided compound each heteroatom of heteroaryl, heterocyclyl, heteroaliphatic, ring (e.g., -Cy-, ring formed by groups taken together (e.g., two R groups taken together) with their intervening atom(s) (if any), etc.), etc., is independently selected from nitrogen, oxygen and sulfur. In some embodiments, in provided compound each heteroatom is independently selected from nitrogen, oxygen, sulfur, silicon and phosphorus. In some embodiments, rings (e.g., cycloaliphatic, aryl, heteroaryl, heterocyclyl, rings formed by groups taken together (e.g., two R groups taken together) with their intervening atom(s) (if any), etc.) contain 1-20 (e.g., 1-15, 1-10, 1-6, etc.) carbon ring atoms and 0-10 heteroatoms. In some embodiments, rings contain 1-10 carbon ring atoms and 0-5 heteroatoms. As described herein, various groups may be optionally substituted. Substituents are routinely utilized in chemistry including in development of various therapeutics. Many substituents can be utilized in accordance with the present disclosure. In some embodiments, a substituent is a hydrocarbon group. In some embodiments, a substituent comprises a heteroatom. In some embodiments, a substituent comprises multiple heteroatoms. In some embodiments, each atom in a substituent is independently selected from hydrogen, carbon, halogen, nitrogen, oxygen, sulfur, phosphorus and silicon. In some embodiments, each atom in a substituent is independently selected from hydrogen, carbon, halogen, nitrogen, oxygen, and sulfur. In some embodiments, each atom in a substituent is independently selected from hydrogen, carbon, fluorine, chlorine, bromine, iodine, nitrogen, oxygen, and sulfur. In some embodiments, the total number of carbon and non-halogen heteroatom(s) in a substituent is about or no more than about 1; in some embodiments, it is no more than about 2; in some embodiments, it is no more than about 3; in some embodiments, it is no more than about 4; in some embodiments, it is no more than about 5; in some embodiments, it is no more than about 6; in some embodiments, it is no more than about 7; in some embodiments, it is no more than about 8; in some embodiments, it is no more than about 9; in some embodiments, it is no more than about 10; in some embodiments, it is no more than about 11; in some embodiments, it is no more than about 12; in some embodiments, it is no more than about 13; in some embodiments, it is no more than about 14; in some embodiments, it is no more than about 15. Various substituents are presented in provided compounds as examples.

In some embodiments, a R^{SUPERSCRIPT} group, wherein SUPERSCRIPT can be any text, e.g., 1, 2, 3, w, etc., is R′ as described herein. In some embodiments, it is R as described herein. In some embodiments, a L^{SUPERSCRIPT} group, wherein SUPERSCRIPT can be any text, e.g., 1, 2, 3, w, etc., is L as described herein.

As described herein, one or more isotopes may be independently enriched, independently at one or more positions. In some embodiments, an enrichment is about or at least about 5%, 10%, 15%, 20%, 25%, 30%, 35%, 40%, 45%, 50%, 60%, 70%, 80%, 85%, 90%, 95%, 99%, 100%, 150%, 200%, 500%, 1000%, 2000%, 5000% more than a natural abundance as applicable. In some embodiments, an enrichment is about or at least about 5, 10, 15, 20, 25, 30, 35, 40, 45, 50, 60, 70, 80, 85, 90, 95, 99, 100, 150, 200, 500, 1000, 2000, 5000 fold more than a natural abundance as applicable. In some embodiments, a level of an isotope at a position is about or at least about 5%, 10%, 15%, 20%, 25%, 30%, 35%, 40%, 45%, 50%, 60%, 70%, 80%, 85%, 90%, 95%, or 99% of all compound molecules in a composition. For example, in some embodiments, designation of an atom as deuterium indicates that for that atom, at least 5% of all compound molecules are deuterated. In some embodiments, about or at least about 10%, 20%, 30%, 40% or 50% all compound molecules are deuterated at designated positions. In some embodiments, a percentage is about or at least about 60%, 70%, 80%, 85%, 90%, 95%, or 99% of all compound molecules. In some embodiments, a compound may have two or more positions deuterated, each of which independently has a percentage of about or at least about 5%, 10%, 15%, 20%, 25%, 30%, 35%, 40%, 45%, 50%, 60%, 70%, 80%, 85%, 90%, 95%, or 99% of all compound molecules in a composition. Various technologies can be utilized to provide enriched levels of isotopes. In some embodiments, reagents having enriched levels of one or more isotopes are utilized in preparation of provided compounds or composition. In some embodiments, an enrich level is about or at least about 5, 10, 15, 20, 25, 30, 35, 40, 45, 50, 60, 70, 80, 85, 90, 95, 99, 100, 150, 200, 500, 1000, 2000, 5000 fold more than a reference preparation prepared without utilizing any reagent that has an enriched level of an isotope.

In some embodiments, a provided compound is selected from below:

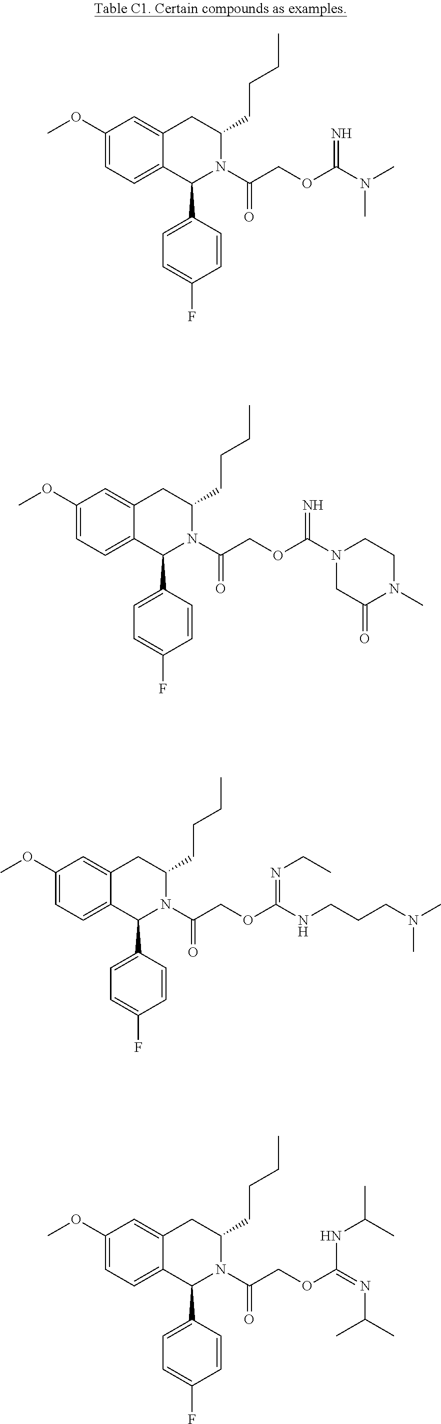

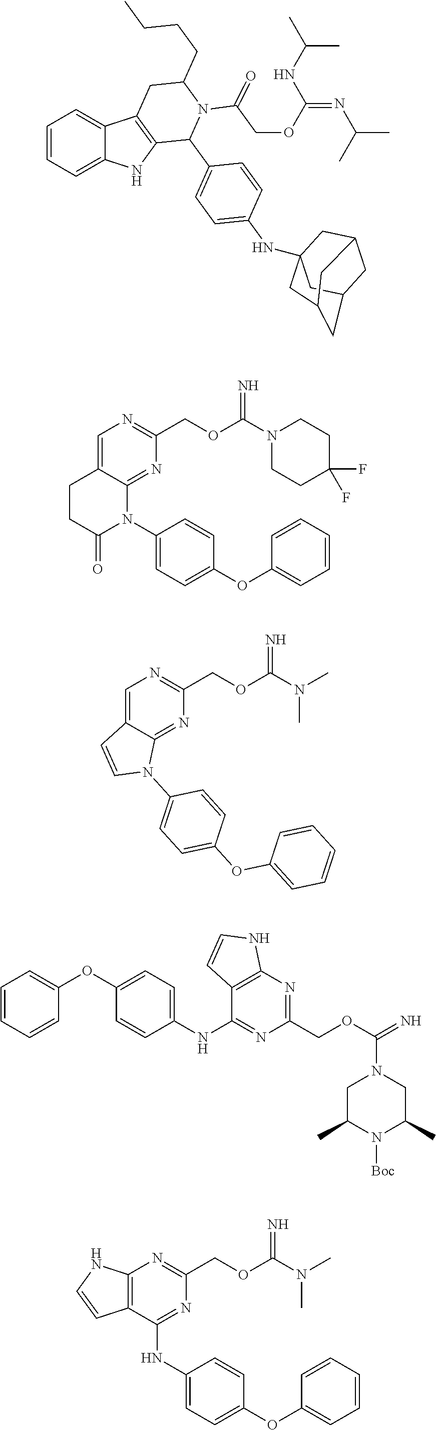 [SMILES: CCCCC1Cc2c([nH]c3ccccc23)C(c2ccc(NC34CC5CC(CC(C5)C3)C4)cc2)N1C(=O)CO/C(=N\C(C)C)NC(C)C ; N=C(OCc1ncc2c(n1)N(c1ccc(Oc3ccccc3)cc1)C(=O)CC2)N1CCC(F)(F)CC1 ; CN(C)C(=N)OCc1ncc2ccn(-c3ccc(Oc4ccccc4)cc3)c2n1 ; C[C@@H]1CN(C(=N)OCc2nc(Nc3ccc(Oc4ccccc4)cc3)c3cc[nH]c3n2)C[C@H](C)N1C(=O)OC(C)(C)C ; CN(C)C(=N)OCc1nc(Nc2ccc(Oc3ccccc3)cc2)c2cc[nH]c2n1]

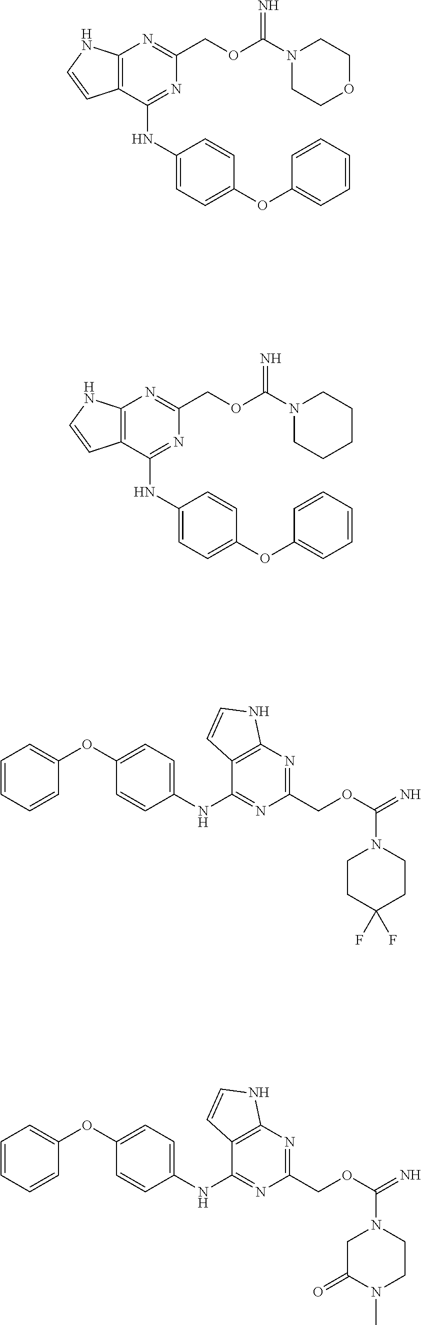 [SMILES: N=C(OCc1nc(Nc2ccc(Oc3ccccc3)cc2)c2cc[nH]c2n1)N1CCOCC1 ; N=C(OCc1nc(Nc2ccc(Oc3ccccc3)cc2)c2cc[nH]c2n1)N1CCCCC1 ; N=C(OCc1nc(Nc2ccc(Oc3ccccc3)cc2)c2cc[nH]c2n1)N1CCC(F)(F)CC1 ; CN1CCN(C(=N)OCc2nc(Nc3ccc(Oc4ccccc4)cc3)c3cc[nH]c3n2)CC1=O]

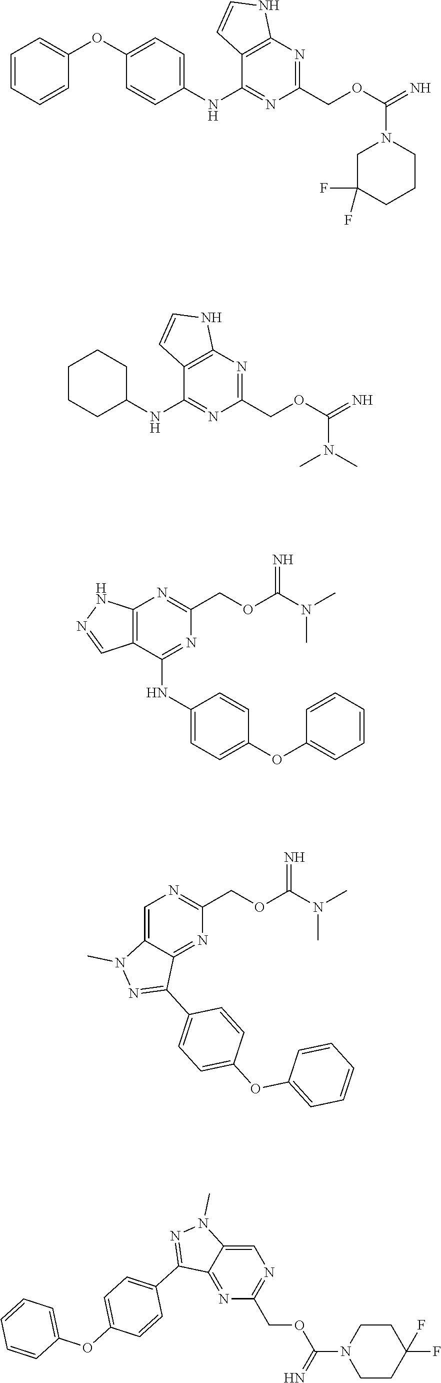

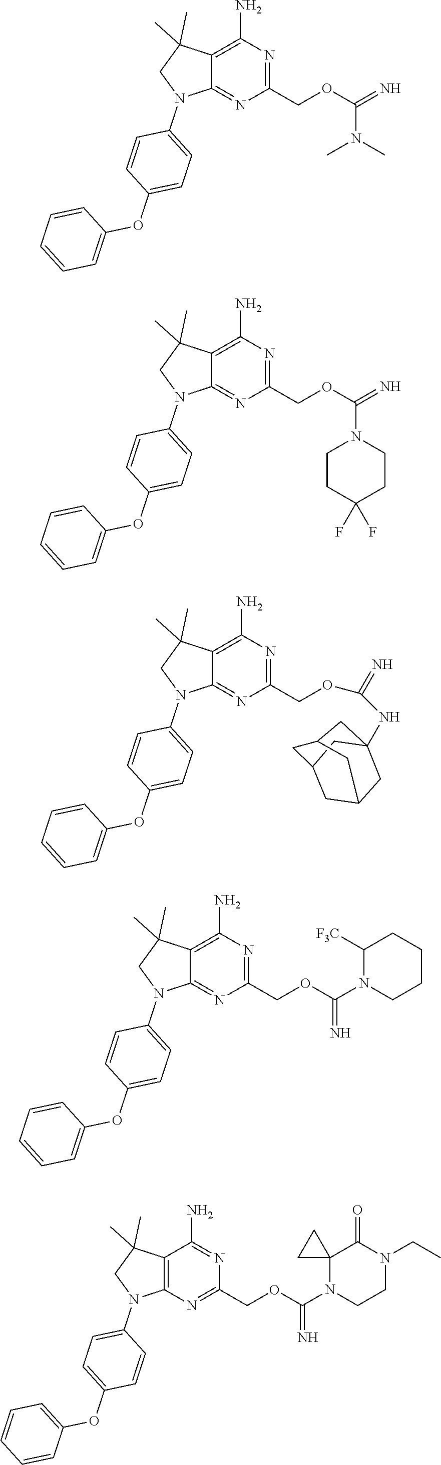 [SMILES: CN(C)C(=N)OCc1nc(N)c2c(n1)N(c1ccc(Oc3ccccc3)cc1)CC2(C)C ; CC1(C)CN(c2ccc(Oc3ccccc3)cc2)c2nc(COC(=N)N3CCC(F)(F)CC3)nc(N)c21 ; CC1(C)CN(c2ccc(Oc3ccccc3)cc2)c2nc(COC(=N)NC34CC5CC(CC(C5)C3)C4)nc(N)c21 ; CC1(C)CN(c2ccc(Oc3ccccc3)cc2)c2nc(COC(=N)N3CCCCC3C(F)(F)F)nc(N)c21 ; CCN1CCN(C(=N)OCc2nc(N)c3c(n2)N(c2ccc(Oc4ccccc4)cc2)CC3(C)C)C2(CC2)C1=O]

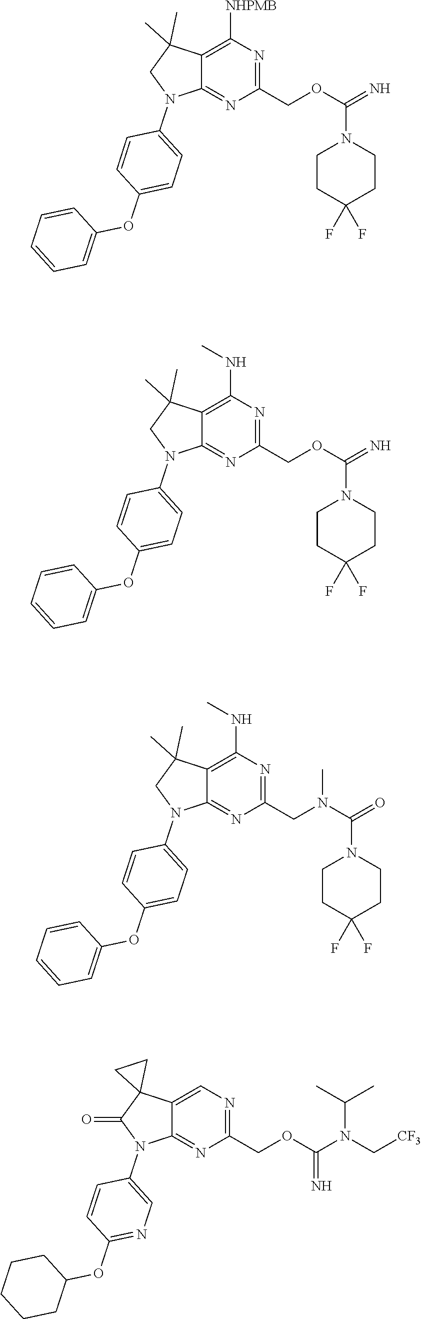

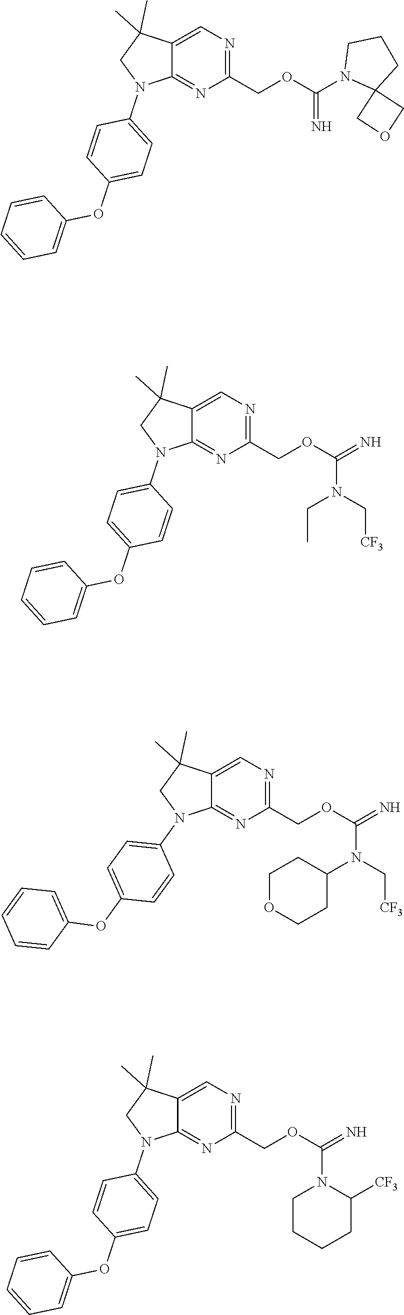 [SMILES: CC1(C)CN(c2ccc(Oc3ccccc3)cc2)c2nc(COC(=N)N3CCCC34COC4)ncc21 ; CCN(CC(F)(F)F)C(=N)OCc1ncc2c(n1)N(c1ccc(Oc3ccccc3)cc1)CC2(C)C ; CC1(C)CN(c2ccc(Oc3ccccc3)cc2)c2nc(COC(=N)N(CC(F)(F)F)C3CCOCC3)ncc21 ; CC1(C)CN(c2ccc(Oc3ccccc3)cc2)c2nc(COC(=N)N3CCCCC3C(F)(F)F)ncc21]

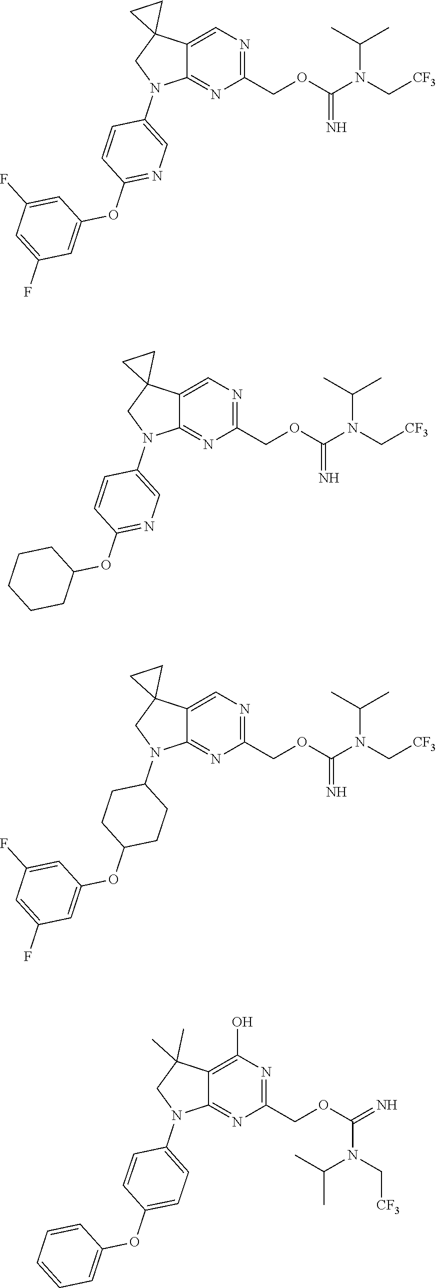 [SMILES: CC(C)N(CC(F)(F)F)C(=N)OCc1ncc2c(n1)N(c1ccc(Oc3cc(F)cc(F)c3)nc1)CC21CC1 ; CC(C)N(CC(F)(F)F)C(=N)OCc1ncc2c(n1)N(c1ccc(OC3CCCCC3)nc1)CC21CC1 ; CC(C)N(CC(F)(F)F)C(=N)OCc1ncc2c(n1)N(C1CCC(Oc3cc(F)cc(F)c3)CC1)CC21CC1 ; CC(C)N(CC(F)(F)F)C(=N)OCc1nc(O)c2c(n1)N(c1ccc(Oc3ccccc3)cc1)CC2(C)C]

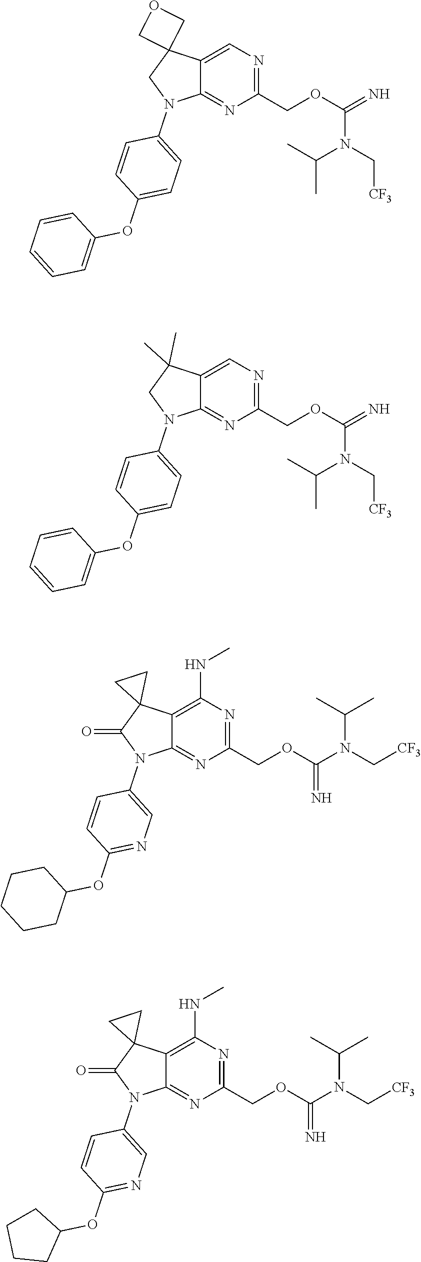 [SMILES: CC(C)N(CC(F)(F)F)C(=N)OCc1ncc2c(n1)N(c1ccc(Oc3ccccc3)cc1)CC21COC1 ; CC(C)N(CC(F)(F)F)C(=N)OCc1ncc2c(n1)N(c1ccc(Oc3ccccc3)cc1)CC2(C)C ; CNc1nc(COC(=N)N(CC(F)(F)F)C(C)C)nc2c1C1(CC1)C(=O)N2c1ccc(OC2CCCCC2)nc1 ; CNc1nc(COC(=N)N(CC(F)(F)F)C(C)C)nc2c1C1(CC1)C(=O)N2c1ccc(OC2CCCC2)nc1]

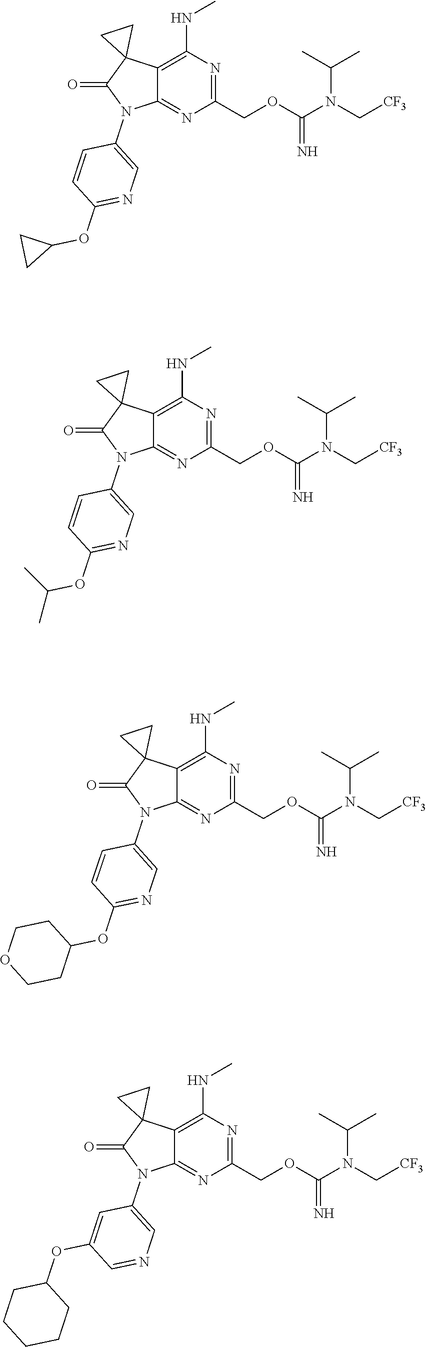 [SMILES: CNc1nc(COC(=N)N(CC(F)(F)F)C(C)C)nc2c1C1(CC1)C(=O)N2c1ccc(OC2CC2)nc1 ; CNc1nc(COC(=N)N(CC(F)(F)F)C(C)C)nc2c1C1(CC1)C(=O)N2c1ccc(OC(C)C)nc1 ; CNc1nc(COC(=N)N(CC(F)(F)F)C(C)C)nc2c1C1(CC1)C(=O)N2c1ccc(OC2CCOCC2)nc1 ; CNc1nc(COC(=N)N(CC(F)(F)F)C(C)C)nc2c1C1(CC1)C(=O)N2c1cncc(OC2CCCCC2)c1]

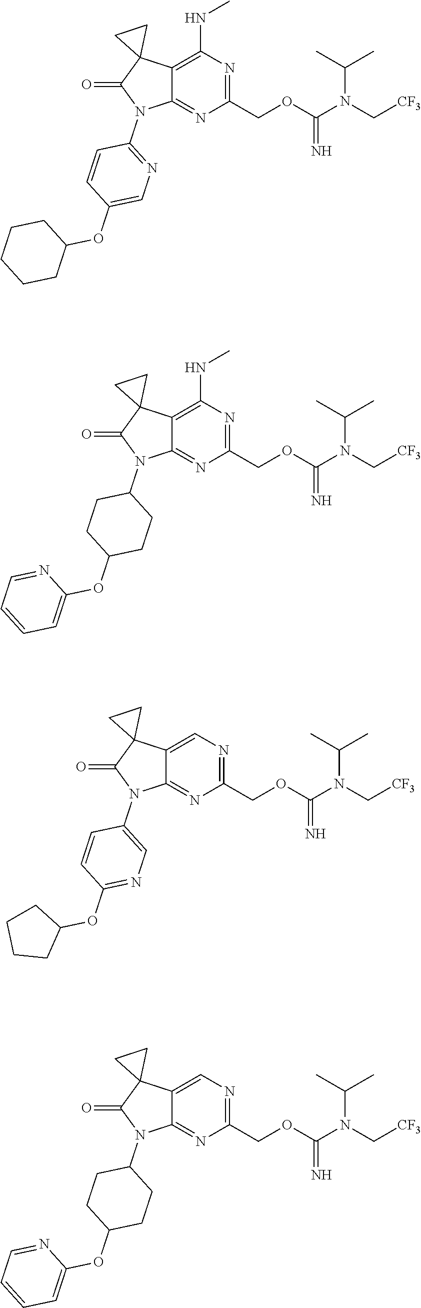 [SMILES: CNc1nc(COC(=N)N(CC(F)(F)F)C(C)C)nc2c1C1(CC1)C(=O)N2c1ccc(OC2CCCCC2)cn1 ; CNc1nc(COC(=N)N(CC(F)(F)F)C(C)C)nc2c1C1(CC1)C(=O)N2C1CCC(Oc2ccccn2)CC1 ; CC(C)N(CC(F)(F)F)C(=N)OCc1ncc2c(n1)N(c1ccc(OC3CCCC3)nc1)C(=O)C21CC1 ; CC(C)N(CC(F)(F)F)C(=N)OCc1ncc2c(n1)N(C1CCC(Oc3ccccn3)CC1)C(=O)C21CC1]

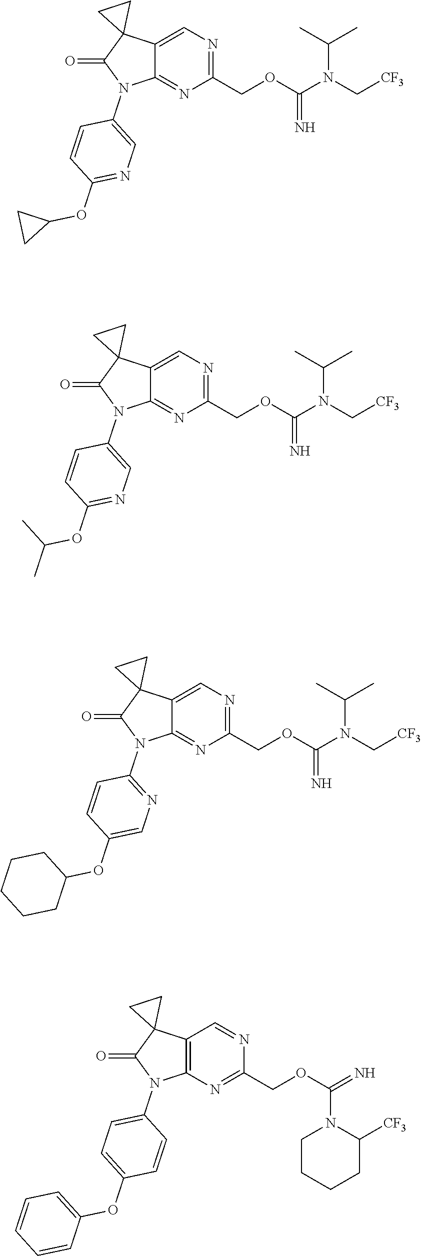 [SMILES: CC(C)N(CC(F)(F)F)C(=N)OCc1ncc2c(n1)N(c1ccc(OC3CC3)nc1)C(=O)C21CC1 ; CC(C)Oc1ccc(N2C(=O)C3(CC3)c3cnc(COC(=N)N(CC(F)(F)F)C(C)C)nc32)cn1 ; CC(C)N(CC(F)(F)F)C(=N)OCc1ncc2c(n1)N(c1ccc(OC3CCCCC3)cn1)C(=O)C21CC1 ; N=C(OCc1ncc2c(n1)N(c1ccc(Oc3ccccc3)cc1)C(=O)C21CC1)N1CCCCC1C(F)(F)F]

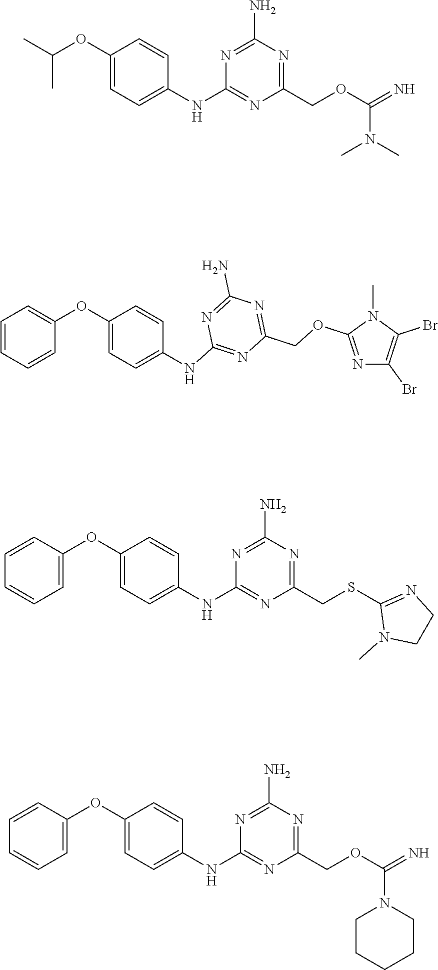 [SMILES: CC(C)Oc1ccc(Nc2nc(N)nc(COC(=N)N(C)C)n2)cc1 ; Cn1c(OCc2nc(N)nc(Nc3ccc(Oc4ccccc4)cc3)n2)nc(Br)c1Br ; CN1CCN=C1SCc1nc(N)nc(Nc2ccc(Oc3ccccc3)cc2)n1 ; N=C(OCc1nc(N)nc(Nc2ccc(Oc3ccccc3)cc2)n1)N1CCCCC1]

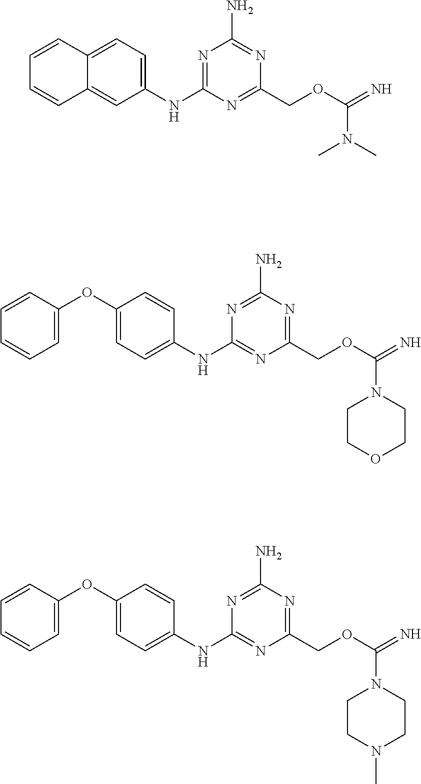 [SMILES: CN(C)C(=N)OCc1nc(N)nc(Nc2ccc3ccccc3c2)n1 ; N=C(OCc1nc(N)nc(Nc2ccc(Oc3ccccc3)cc2)n1)N1CCOCC1 ; CN1CCN(C(=N)OCc2nc(N)nc(Nc3ccc(Oc4ccccc4)cc3)n2)CC1]

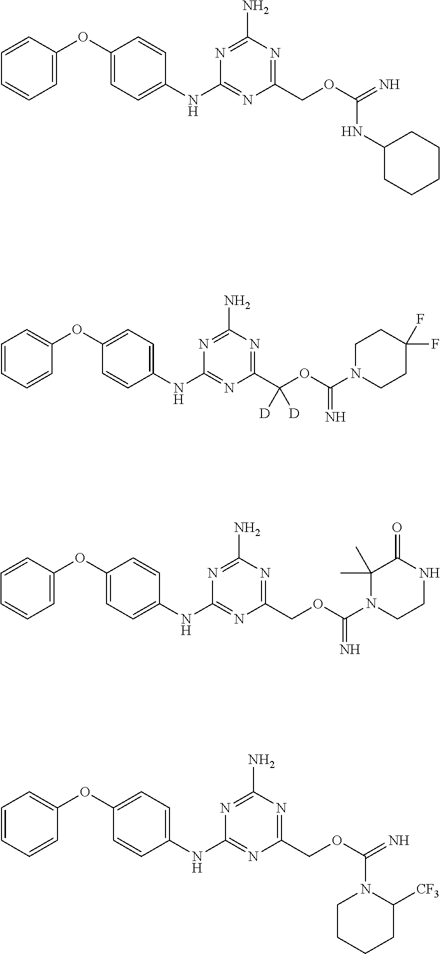 [SMILES: N=C(NC1CCCCC1)OCc1nc(N)nc(Nc2ccc(Oc3ccccc3)cc2)n1 ; [2H]C([2H])(OC(=N)N1CCC(F)(F)CC1)c1nc(N)nc(Nc2ccc(Oc3ccccc3)cc2)n1 ; CC1(C)C(=O)NCCN1C(=N)OCc1nc(N)nc(Nc2ccc(Oc3ccccc3)cc2)n1 ; N=C(OCc1nc(N)nc(Nc2ccc(Oc3ccccc3)cc2)n1)N1CCCCC1C(F)(F)F]

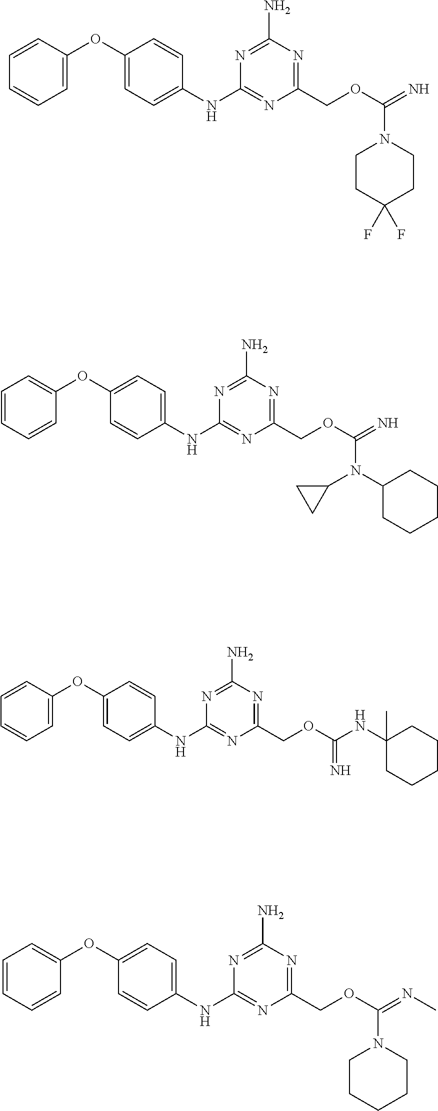 [SMILES: N=C(OCc1nc(N)nc(Nc2ccc(Oc3ccccc3)cc2)n1)N1CCC(F)(F)CC1 ; N=C(OCc1nc(N)nc(Nc2ccc(Oc3ccccc3)cc2)n1)N(C1CCCCC1)C1CC1 ; CC1(NC(=N)OCc2nc(N)nc(Nc3ccc(Oc4ccccc4)cc3)n2)CCCCC1 ; C/N=C(/OCc1nc(N)nc(Nc2ccc(Oc3ccccc3)cc2)n1)N1CCCCC1]

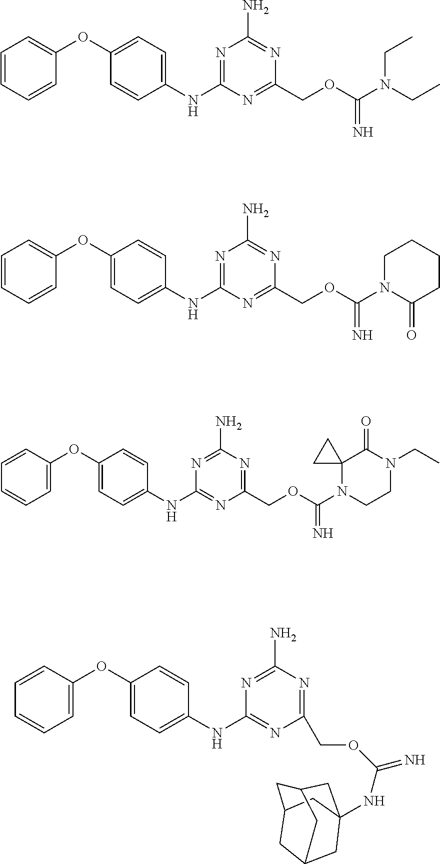 [SMILES: CCN(CC)C(=N)OCc1nc(N)nc(Nc2ccc(Oc3ccccc3)cc2)n1 ; N=C(OCc1nc(N)nc(Nc2ccc(Oc3ccccc3)cc2)n1)N1CCCCC1=O ; CCN1CCN(C(=N)OCc2nc(N)nc(Nc3ccc(Oc4ccccc4)cc3)n2)C2(CC2)C1=O ; N=C(NC12CC3CC(CC(C3)C1)C2)OCc1nc(N)nc(Nc2ccc(Oc3ccccc3)cc2)n1]

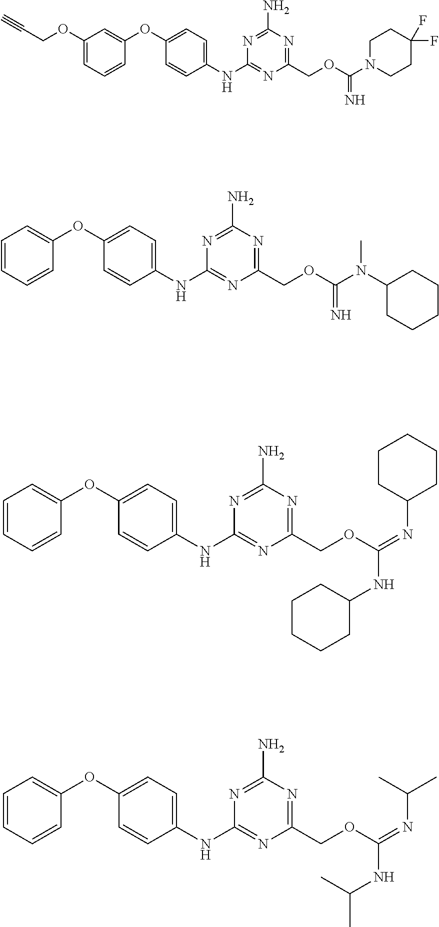 [SMILES: C#CCOc1cccc(Oc2ccc(Nc3nc(N)nc(COC(=N)N4CCC(F)(F)CC4)n3)cc2)c1 ; CN(C(=N)OCc1nc(N)nc(Nc2ccc(Oc3ccccc3)cc2)n1)C1CCCCC1 ; Nc1nc(CO/C(=N\C2CCCCC2)NC2CCCCC2)nc(Nc2ccc(Oc3ccccc3)cc2)n1 ; CC(C)/N=C(/NC(C)C)OCc1nc(N)nc(Nc2ccc(Oc3ccccc3)cc2)n1]

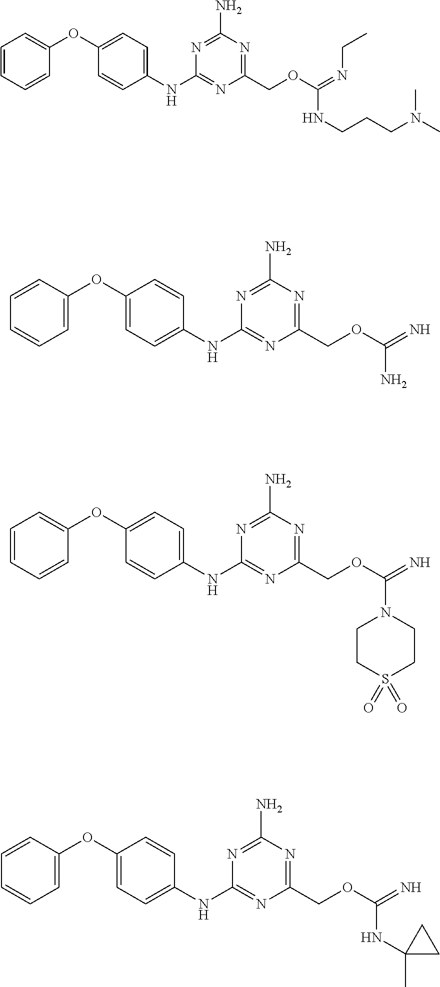 [SMILES: CC/N=C(/NCCCN(C)C)OCc1nc(N)nc(Nc2ccc(Oc3ccccc3)cc2)n1 ; N=C(N)OCc1nc(N)nc(Nc2ccc(Oc3ccccc3)cc2)n1 ; N=C(OCc1nc(N)nc(Nc2ccc(Oc3ccccc3)cc2)n1)N1CCS(=O)(=O)CC1 ; CC1(NC(=N)OCc2nc(N)nc(Nc3ccc(Oc4ccccc4)cc3)n2)CC1]

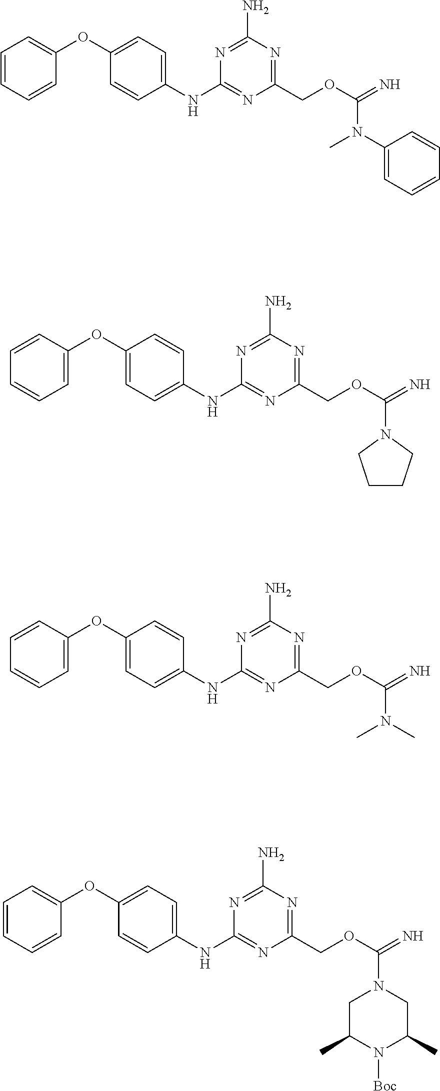 [SMILES: CN(C(=N)OCc1nc(N)nc(Nc2ccc(Oc3ccccc3)cc2)n1)c1ccccc1 ; N=C(OCc1nc(N)nc(Nc2ccc(Oc3ccccc3)cc2)n1)N1CCCC1 ; CN(C)C(=N)OCc1nc(N)nc(Nc2ccc(Oc3ccccc3)cc2)n1 ; C[C@@H]1CN(C(=N)OCc2nc(N)nc(Nc3ccc(Oc4ccccc4)cc3)n2)C[C@H](C)N1C(=O)OC(C)(C)C]

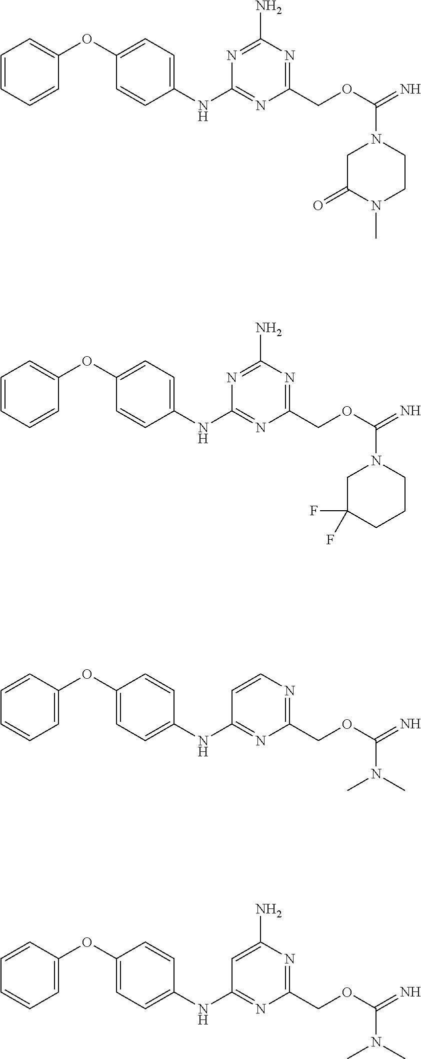 [SMILES: CN1CCN(C(=N)OCc2nc(N)nc(Nc3ccc(Oc4ccccc4)cc3)n2)CC1=O ; N=C(OCc1nc(N)nc(Nc2ccc(Oc3ccccc3)cc2)n1)N1CCCC(F)(F)C1 ; CN(C)C(=N)OCc1nccc(Nc2ccc(Oc3ccccc3)cc2)n1 ; CN(C)C(=N)OCc1nc(N)cc(Nc2ccc(Oc3ccccc3)cc2)n1]

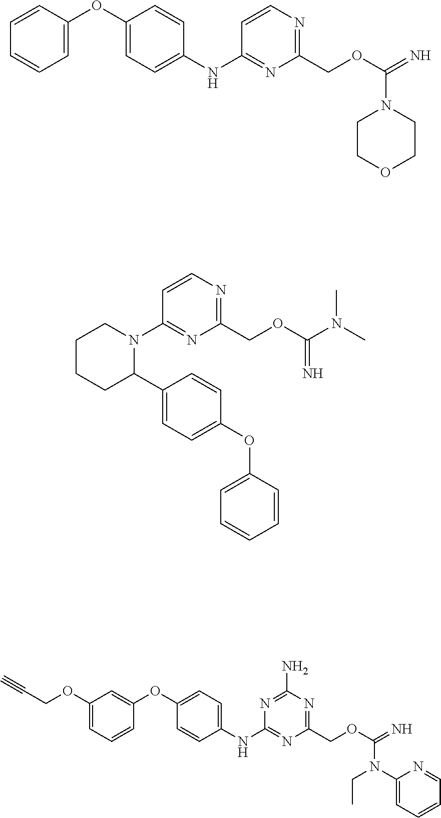

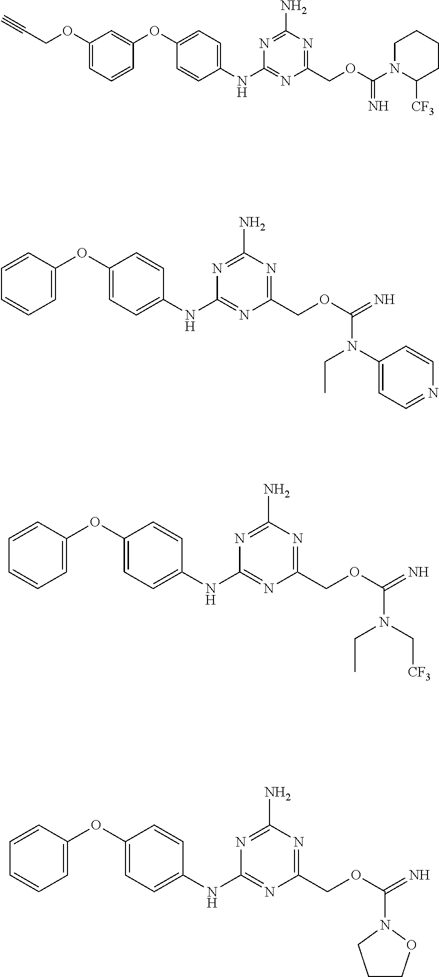 [SMILES: C#CCOc1cccc(Oc2ccc(Nc3nc(N)nc(COC(=N)N4CCCCC4C(F)(F)F)n3)cc2)c1 ; CCN(C(=N)OCc1nc(N)nc(Nc2ccc(Oc3ccccc3)cc2)n1)c1ccncc1 ; CCN(CC(F)(F)F)C(=N)OCc1nc(N)nc(Nc2ccc(Oc3ccccc3)cc2)n1 ; N=C(OCc1nc(N)nc(Nc2ccc(Oc3ccccc3)cc2)n1)N1CCCO1]

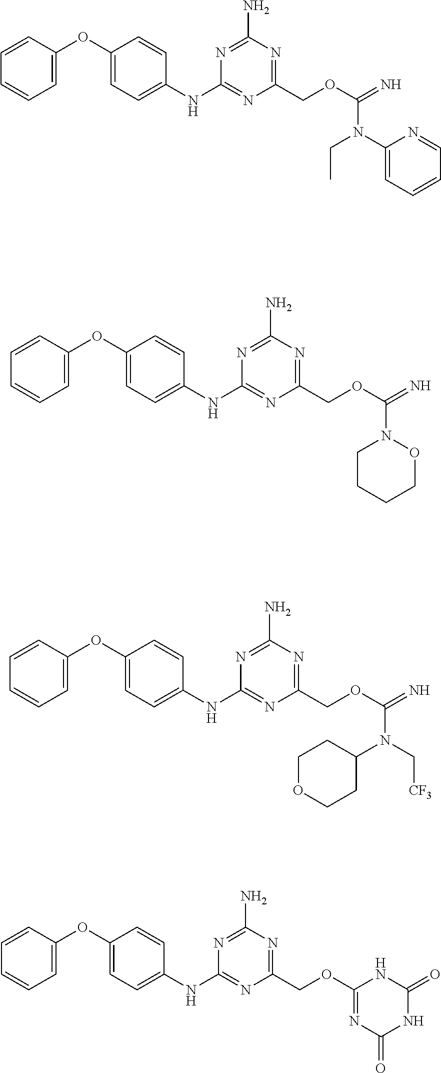 [SMILES: CCN(C(=N)OCc1nc(N)nc(Nc2ccc(Oc3ccccc3)cc2)n1)c1ccccn1 ; N=C(OCc1nc(N)nc(Nc2ccc(Oc3ccccc3)cc2)n1)N1CCCCO1 ; N=C(OCc1nc(N)nc(Nc2ccc(Oc3ccccc3)cc2)n1)N(CC(F)(F)F)C1CCOCC1 ; Nc1nc(COc2nc(=O)[nH]c(=O)[nH]2)nc(Nc2ccc(Oc3ccccc3)cc2)n1]

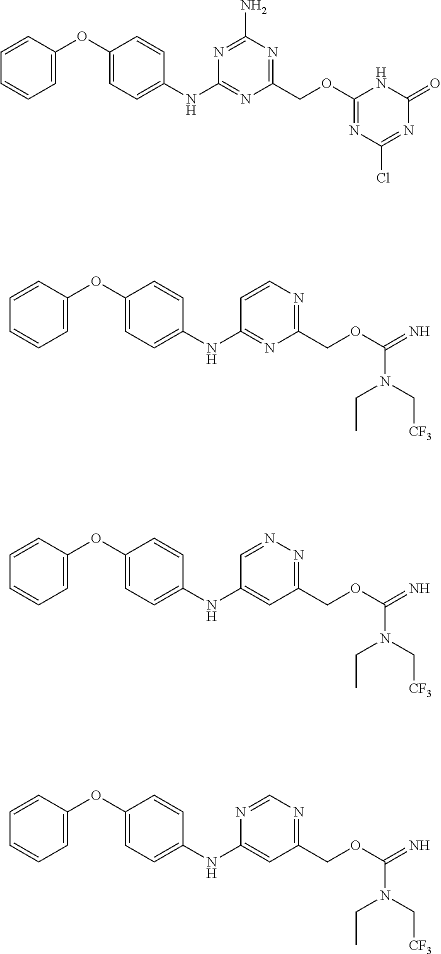 [SMILES: Nc1nc(COc2nc(Cl)nc(=O)[nH]2)nc(Nc2ccc(Oc3ccccc3)cc2)n1 ; CCN(CC(F)(F)F)C(=N)OCc1nccc(Nc2ccc(Oc3ccccc3)cc2)n1 ; CCN(CC(F)(F)F)C(=N)OCc1cc(Nc2ccc(Oc3ccccc3)cc2)cnn1 ; CCN(CC(F)(F)F)C(=N)OCc1cc(Nc2ccc(Oc3ccccc3)cc2)ncn1]

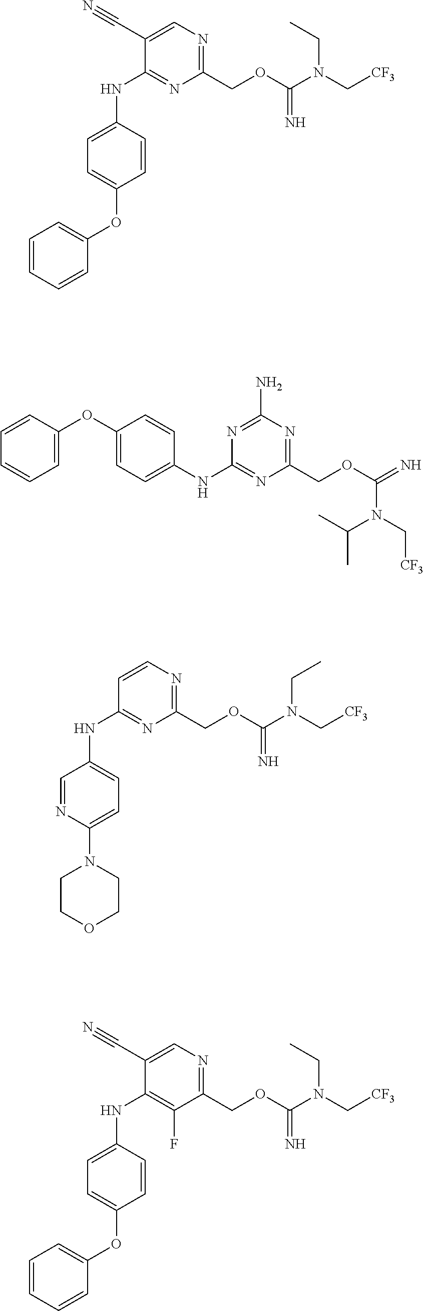 [SMILES: CCN(CC(F)(F)F)C(=N)OCc1ncc(C#N)c(Nc2ccc(Oc3ccccc3)cc2)n1 ; CC(C)N(CC(F)(F)F)C(=N)OCc1nc(N)nc(Nc2ccc(Oc3ccccc3)cc2)n1 ; CCN(CC(F)(F)F)C(=N)OCc1nccc(Nc2ccc(N3CCOCC3)nc2)n1 ; CCN(CC(F)(F)F)C(=N)OCc1ncc(C#N)c(Nc2ccc(Oc3ccccc3)cc2)c1F]

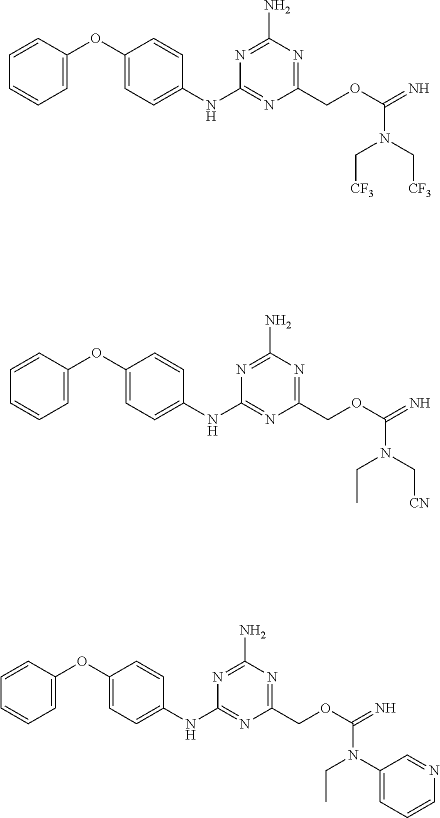 [SMILES: N=C(OCc1nc(N)nc(Nc2ccc(Oc3ccccc3)cc2)n1)N(CC(F)(F)F)CC(F)(F)F ; CCN(CC#N)C(=N)OCc1nc(N)nc(Nc2ccc(Oc3ccccc3)cc2)n1 ; CCN(C(=N)OCc1nc(N)nc(Nc2ccc(Oc3ccccc3)cc2)n1)c1cccnc1]

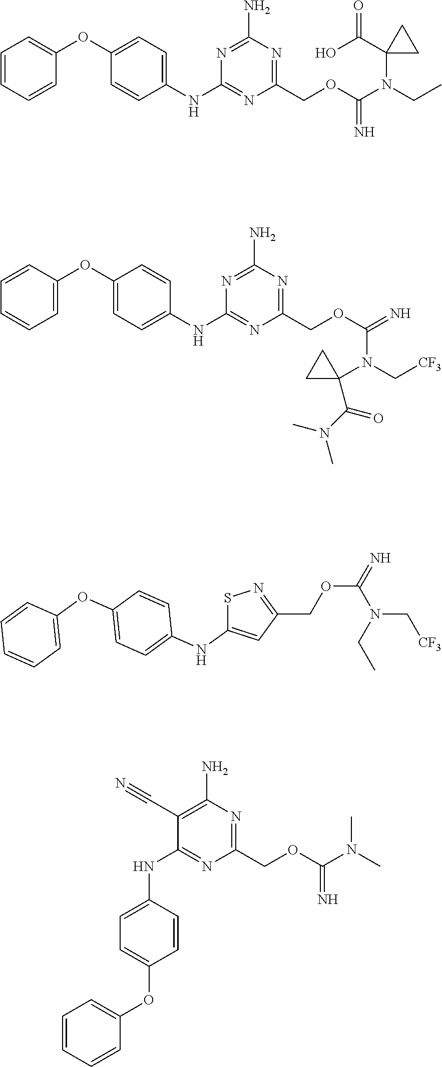 [SMILES: CCN(C(=N)OCc1nc(N)nc(Nc2ccc(Oc3ccccc3)cc2)n1)C1(C(=O)O)CC1 ; CN(C)C(=O)C1(N(CC(F)(F)F)C(=N)OCc2nc(N)nc(Nc3ccc(Oc4ccccc4)cc3)n2)CC1 ; CCN(CC(F)(F)F)C(=N)OCc1cc(Nc2ccc(Oc3ccccc3)cc2)sn1 ; CN(C)C(=N)OCc1nc(N)c(C#N)c(Nc2ccc(Oc3ccccc3)cc2)n1]

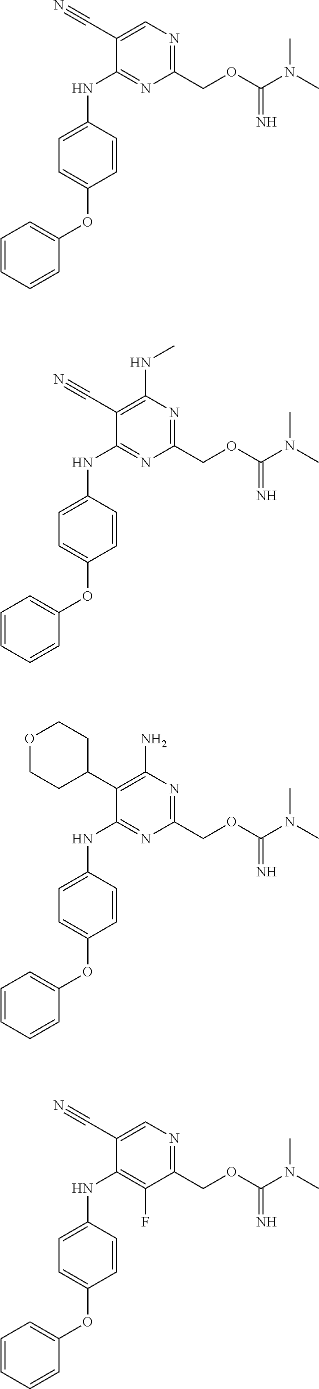 [SMILES: CN(C)C(=N)OCc1ncc(C#N)c(Nc2ccc(Oc3ccccc3)cc2)n1 ; CNc1nc(COC(=N)N(C)C)nc(Nc2ccc(Oc3ccccc3)cc2)c1C#N ; CN(C)C(=N)OCc1nc(N)c(C2CCOCC2)c(Nc2ccc(Oc3ccccc3)cc2)n1 ; CN(C)C(=N)OCc1ncc(C#N)c(Nc2ccc(Oc3ccccc3)cc2)c1F]

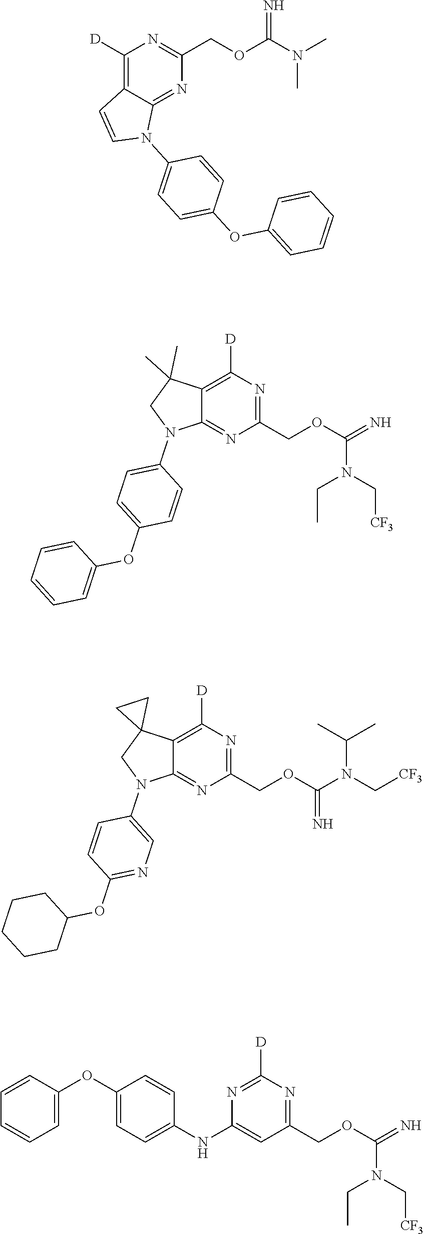 [SMILES: [2H]c1nc(COC(=N)N(C)C)nc2c1ccn2-c1ccc(Oc2ccccc2)cc1 ; [2H]c1nc(COC(=N)N(CC)CC(F)(F)F)nc2c1C(C)(C)CN2c1ccc(Oc2ccccc2)cc1 ; [2H]c1nc(COC(=N)N(CC(F)(F)F)C(C)C)nc2c1C1(CC1)CN2c1ccc(OC2CCCCC2)nc1 ; [2H]c1nc(COC(=N)N(CC)CC(F)(F)F)cc(Nc2ccc(Oc3ccccc3)cc2)n1]

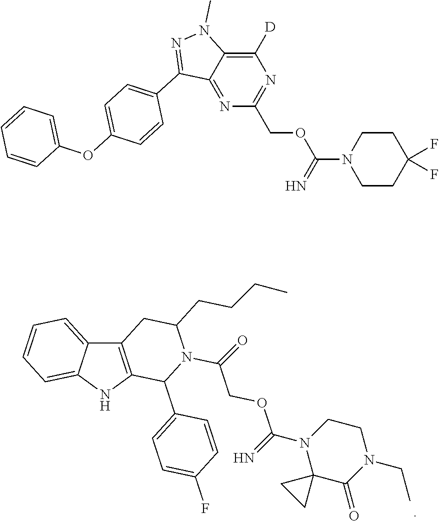 [SMILES: [2H]c1nc(COC(=N)N2CCC(F)(F)CC2)nc2c(-c3ccc(Oc4ccccc4)cc3)nn(C)c12 ; CCCCC1Cc2c([nH]c3ccccc23)C(c2ccc(F)cc2)N1C(=O)COC(=N)N1CCN(CC)C(=O)C12CC2]

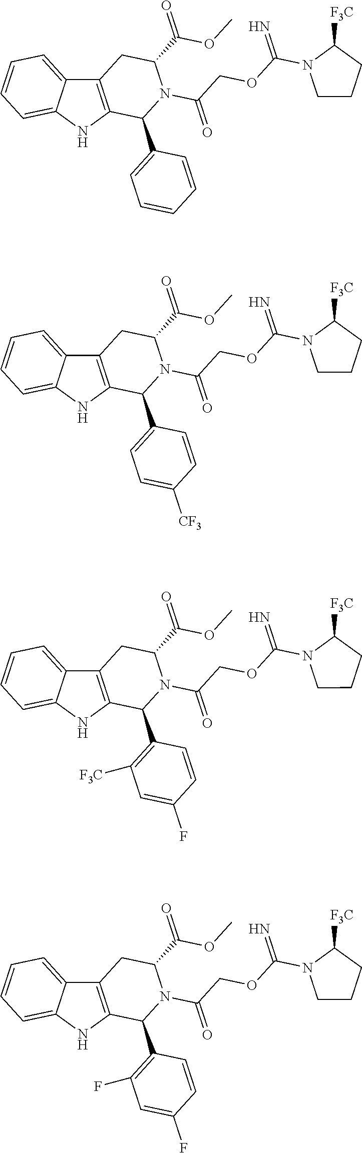 [SMILES: COC(=O)[C@H]1Cc2c([nH]c3ccccc23)[C@H](c2ccccc2)N1C(=O)COC(=N)N1CCC[C@@H]1C(F)(F)F ; COC(=O)[C@H]1Cc2c([nH]c3ccccc23)[C@H](c2ccc(C(F)(F)F)cc2)N1C(=O)COC(=N)N1CCC[C@@H]1C(F)(F)F ; COC(=O)[C@H]1Cc2c([nH]c3ccccc23)[C@H](c2ccc(F)cc2C(F)(F)F)N1C(=O)COC(=N)N1CCC[C@@H]1C(F)(F)F ; COC(=O)[C@H]1Cc2c([nH]c3ccccc23)[C@H](c2ccc(F)cc2F)N1C(=O)COC(=N)N1CCC[C@@H]1C(F)(F)F]

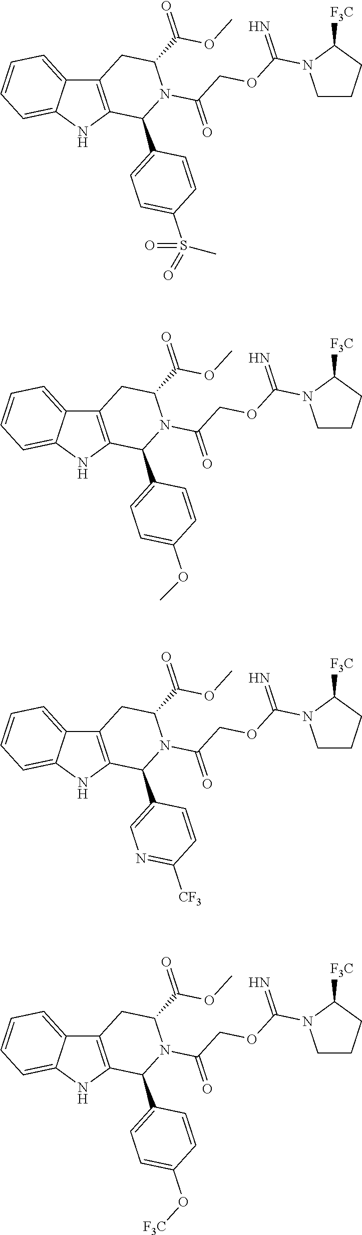 [SMILES: COC(=O)[C@H]1Cc2c([nH]c3ccccc23)[C@H](c2ccc(S(C)(=O)=O)cc2)N1C(=O)COC(=N)N1CCC[C@@H]1C(F)(F)F ; COC(=O)[C@H]1Cc2c([nH]c3ccccc23)[C@H](c2ccc(OC)cc2)N1C(=O)COC(=N)N1CCC[C@@H]1C(F)(F)F ; COC(=O)[C@H]1Cc2c([nH]c3ccccc23)[C@H](c2ccc(C(F)(F)F)nc2)N1C(=O)COC(=N)N1CCC[C@@H]1C(F)(F)F ; COC(=O)[C@H]1Cc2c([nH]c3ccccc23)[C@H](c2ccc(OC(F)(F)F)cc2)N1C(=O)COC(=N)N1CCC[C@@H]1C(F)(F)F]

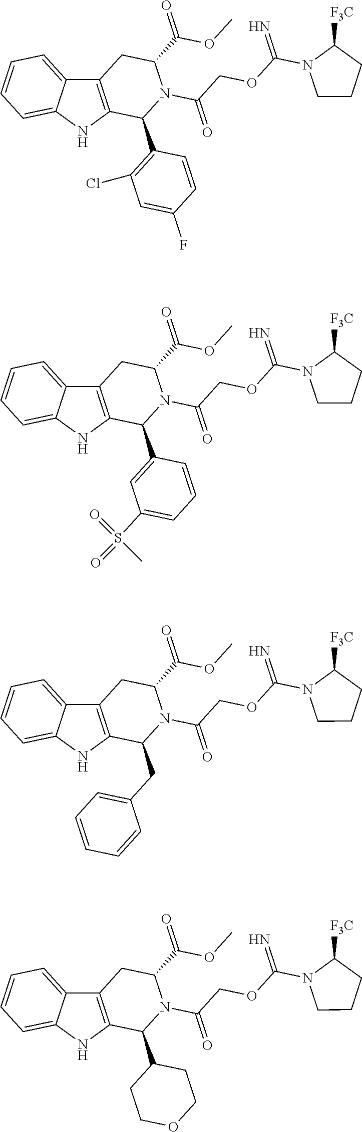 [SMILES: COC(=O)[C@H]1Cc2c([nH]c3ccccc23)[C@H](c2ccc(F)cc2Cl)N1C(=O)COC(=N)N1CCC[C@@H]1C(F)(F)F ; COC(=O)[C@H]1Cc2c([nH]c3ccccc23)[C@H](c2cccc(S(C)(=O)=O)c2)N1C(=O)COC(=N)N1CCC[C@@H]1C(F)(F)F ; COC(=O)[C@H]1Cc2c([nH]c3ccccc23)[C@H](Cc2ccccc2)N1C(=O)COC(=N)N1CCC[C@@H]1C(F)(F)F ; COC(=O)[C@H]1Cc2c([nH]c3ccccc23)[C@H](C2CCOCC2)N1C(=O)COC(=N)N1CCC[C@@H]1C(F)(F)F]

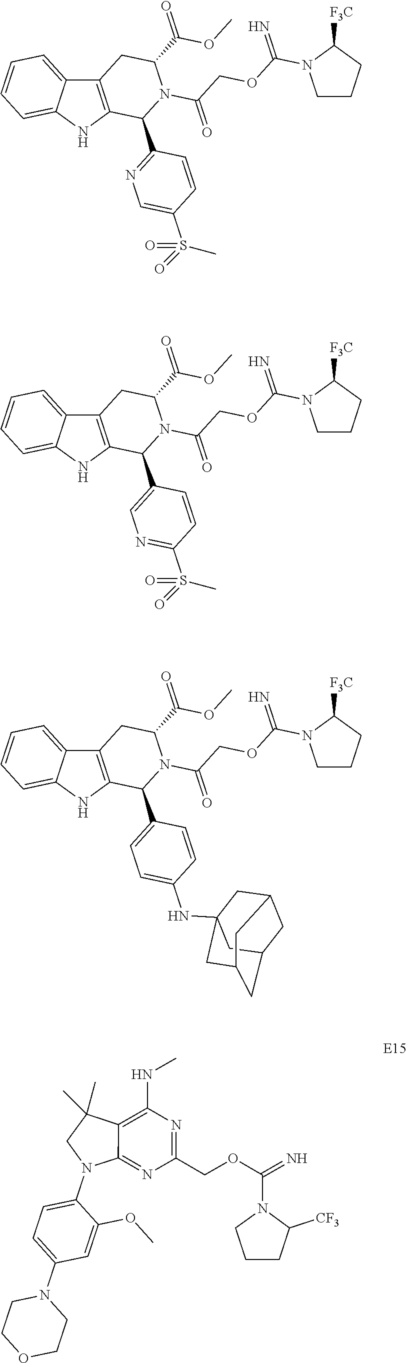 [SMILES: COC(=O)[C@H]1Cc2c([nH]c3ccccc23)[C@H](c2ccc(S(C)(=O)=O)cn2)N1C(=O)COC(=N)N1CCC[C@@H]1C(F)(F)F ; COC(=O)[C@H]1Cc2c([nH]c3ccccc23)[C@H](c2ccc(S(C)(=O)=O)nc2)N1C(=O)COC(=N)N1CCC[C@@H]1C(F)(F)F ; COC(=O)[C@H]1Cc2c([nH]c3ccccc23)[C@H](c2ccc(NC34CC5CC(CC(C5)C3)C4)cc2)N1C(=O)COC(=N)N1CCC[C@@H]1C(F)(F)F ; CNc1nc(COC(=N)N2CCCC2C(F)(F)F)nc2c1C(C)(C)CN2c1ccc(N2CCOCC2)cc1OC]

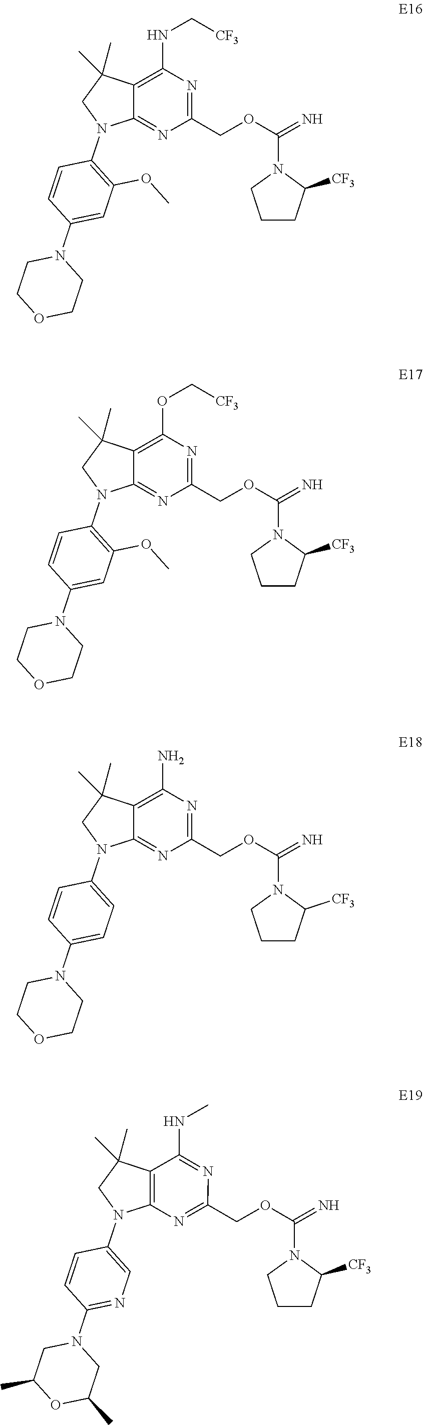 [SMILES: COc1cc(N2CCOCC2)ccc1N1CC(C)(C)c2c(NCC(F)(F)F)nc(COC(=N)N3CCC[C@@H]3C(F)(F)F)nc21 ; COc1cc(N2CCOCC2)ccc1N1CC(C)(C)c2c(OCC(F)(F)F)nc(COC(=N)N3CCC[C@@H]3C(F)(F)F)nc21 ; CC1(C)CN(c2ccc(N3CCOCC3)cc2)c2nc(COC(=N)N3CCCC3C(F)(F)F)nc(N)c21 ; CNc1nc(COC(=N)N2CCC[C@@H]2C(F)(F)F)nc2c1C(C)(C)CN2c1ccc(N2C[C@@H](C)O[C@@H](C)C2)nc1]

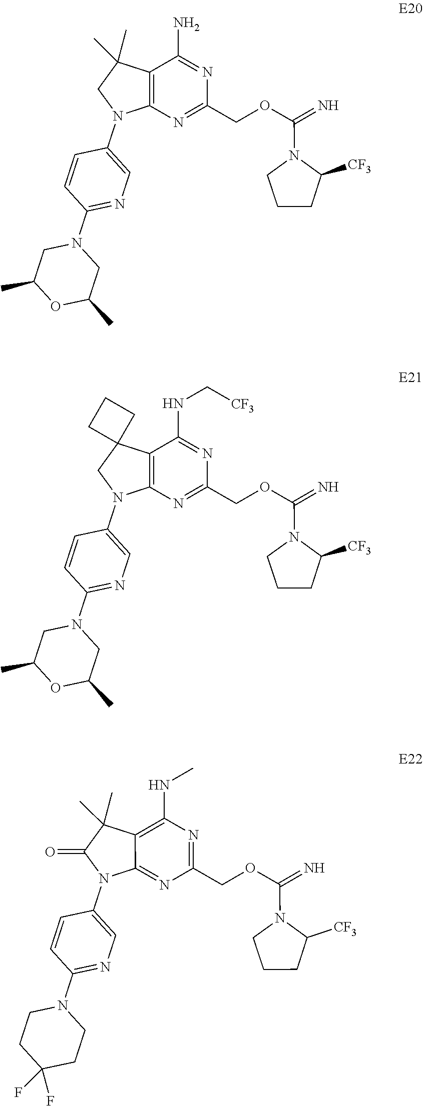 [SMILES: C[C@@H]1CN(c2ccc(N3CC(C)(C)c4c(N)nc(COC(=N)N5CCC[C@@H]5C(F)(F)F)nc43)cn2)C[C@H](C)O1 ; C[C@@H]1CN(c2ccc(N3CC4(CCC4)c4c(NCC(F)(F)F)nc(COC(=N)N5CCC[C@@H]5C(F)(F)F)nc43)cn2)C[C@H](C)O1 ; CNc1nc(COC(=N)N2CCCC2C(F)(F)F)nc2c1C(C)(C)C(=O)N2c1ccc(N2CCC(F)(F)CC2)nc1]

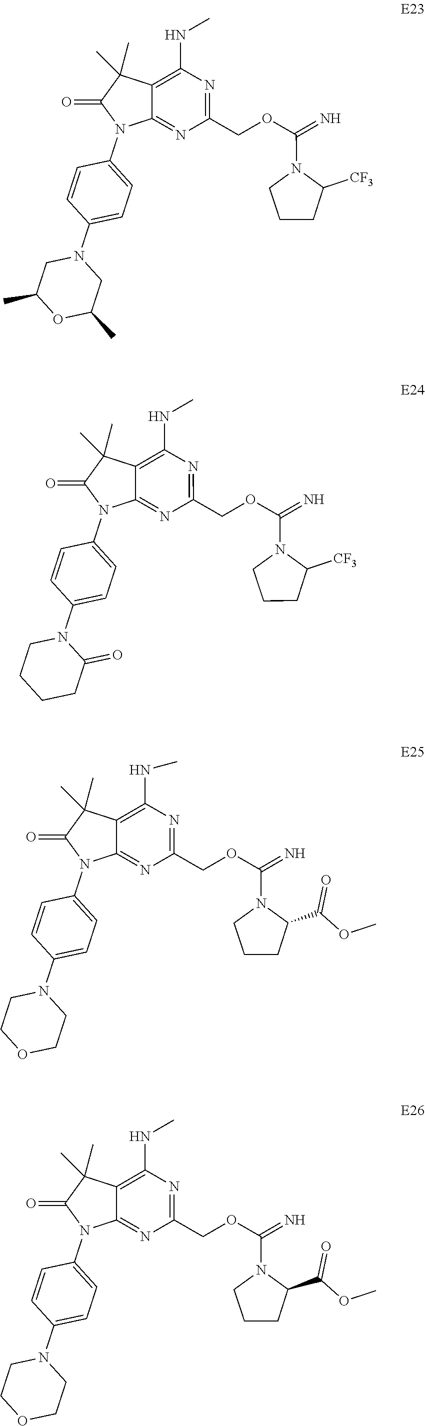 [SMILES: CNc1nc(COC(=N)N2CCCC2C(F)(F)F)nc2c1C(C)(C)C(=O)N2c1ccc(N2C[C@@H](C)O[C@@H](C)C2)cc1 ; CNc1nc(COC(=N)N2CCCC2C(F)(F)F)nc2c1C(C)(C)C(=O)N2c1ccc(N2CCCCC2=O)cc1 ; CNc1nc(COC(=N)N2CCC[C@H]2C(=O)OC)nc2c1C(C)(C)C(=O)N2c1ccc(N2CCOCC2)cc1 ; CNc1nc(COC(=N)N2CCC[C@@H]2C(=O)OC)nc2c1C(C)(C)C(=O)N2c1ccc(N2CCOCC2)cc1]

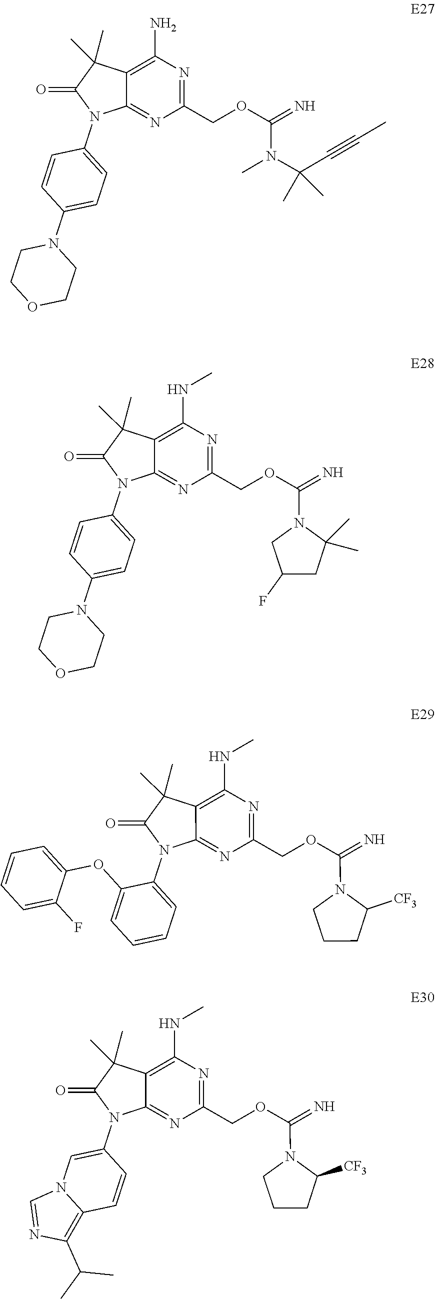 [SMILES: CC#CC(C)(C)N(C)C(=N)OCc1nc(N)c2c(n1)N(c1ccc(N3CCOCC3)cc1)C(=O)C2(C)C ; CNc1nc(COC(=N)N2CC(F)CC2(C)C)nc2c1C(C)(C)C(=O)N2c1ccc(N2CCOCC2)cc1 ; CNc1nc(COC(=N)N2CCCC2C(F)(F)F)nc2c1C(C)(C)C(=O)N2c1ccccc1Oc1ccccc1F ; CNc1nc(COC(=N)N2CCC[C@@H]2C(F)(F)F)nc2c1C(C)(C)C(=O)N2c1ccc2c(C(C)C)ncn2c1]

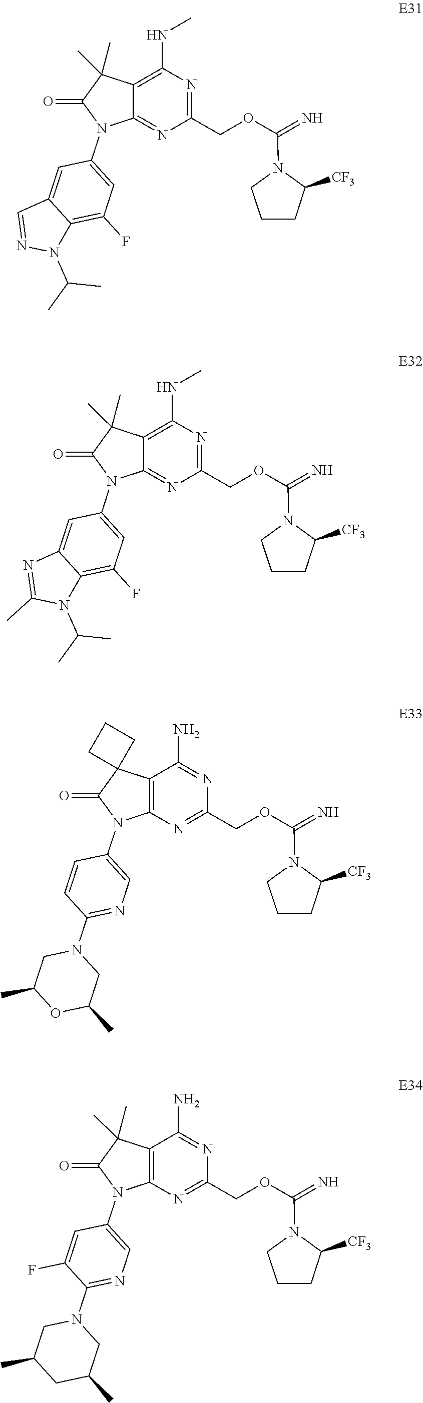 [SMILES: CNc1nc(COC(=N)N2CCC[C@@H]2C(F)(F)F)nc2c1C(C)(C)C(=O)N2c1cc(F)c2c(cnn2C(C)C)c1 ; CNc1nc(COC(=N)N2CCC[C@@H]2C(F)(F)F)nc2c1C(C)(C)C(=O)N2c1cc(F)c2c(c1)nc(C)n2C(C)C ; C[C@@H]1CN(c2ccc(N3C(=O)C4(CCC4)c4c(N)nc(COC(=N)N5CCC[C@@H]5C(F)(F)F)nc43)cn2)C[C@H](C)O1 ; C[C@@H]1C[C@H](C)CN(c2ncc(N3C(=O)C(C)(C)c4c(N)nc(COC(=N)N5CCC[C@@H]5C(F)(F)F)nc43)cc2F)C1]

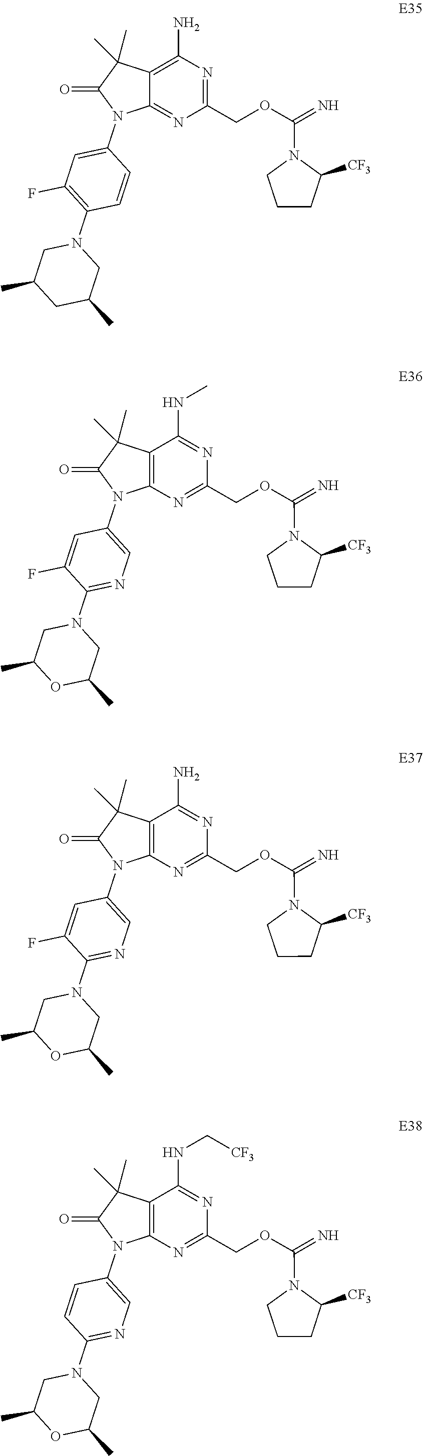 [SMILES: C[C@@H]1C[C@H](C)CN(c2ccc(N3C(=O)C(C)(C)c4c(N)nc(COC(=N)N5CCC[C@@H]5C(F)(F)F)nc43)cc2F)C1 ; CNc1nc(COC(=N)N2CCC[C@@H]2C(F)(F)F)nc2c1C(C)(C)C(=O)N2c1cnc(N2C[C@@H](C)O[C@@H](C)C2)c(F)c1 ; C[C@@H]1CN(c2ncc(N3C(=O)C(C)(C)c4c(N)nc(COC(=N)N5CCC[C@@H]5C(F)(F)F)nc43)cc2F)C[C@H](C)O1 ; C[C@@H]1CN(c2ccc(N3C(=O)C(C)(C)c4c(NCC(F)(F)F)nc(COC(=N)N5CCC[C@@H]5C(F)(F)F)nc43)cn2)C[C@H](C)O1]

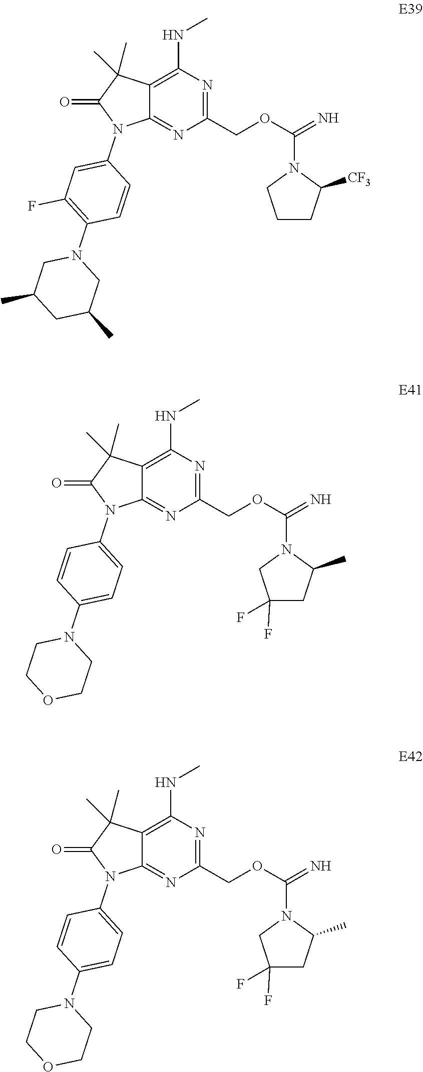 [SMILES: CNc1nc(COC(=N)N2CCC[C@@H]2C(F)(F)F)nc2c1C(C)(C)C(=O)N2c1ccc(N2C[C@H](C)C[C@H](C)C2)c(F)c1 ; CNc1nc(COC(=N)N2CC(F)(F)C[C@@H]2C)nc2c1C(C)(C)C(=O)N2c1ccc(N2CCOCC2)cc1 ; CNc1nc(COC(=N)N2CC(F)(F)C[C@H]2C)nc2c1C(C)(C)C(=O)N2c1ccc(N2CCOCC2)cc1]

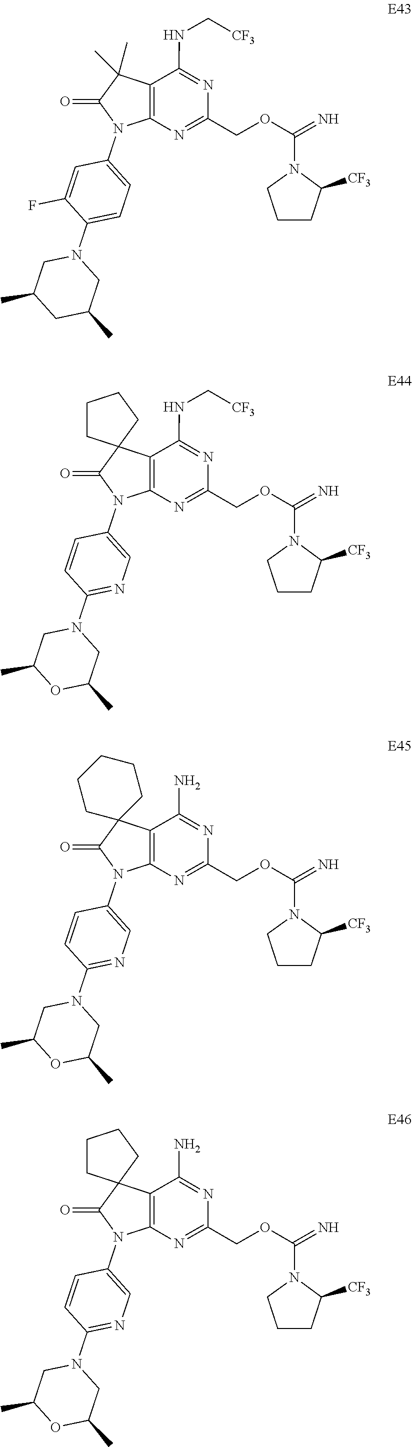 [SMILES: C[C@@H]1C[C@H](C)CN(c2ccc(N3C(=O)C(C)(C)c4c(NCC(F)(F)F)nc(COC(=N)N5CCC[C@@H]5C(F)(F)F)nc43)cc2F)C1 ; C[C@@H]1CN(c2ccc(N3C(=O)C4(CCCC4)c4c(NCC(F)(F)F)nc(COC(=N)N5CCC[C@@H]5C(F)(F)F)nc43)cn2)C[C@H](C)O1 ; C[C@@H]1CN(c2ccc(N3C(=O)C4(CCCCC4)c4c(N)nc(COC(=N)N5CCC[C@@H]5C(F)(F)F)nc43)cn2)C[C@H](C)O1 ; C[C@@H]1CN(c2ccc(N3C(=O)C4(CCCC4)c4c(N)nc(COC(=N)N5CCC[C@@H]5C(F)(F)F)nc43)cn2)C[C@H](C)O1]

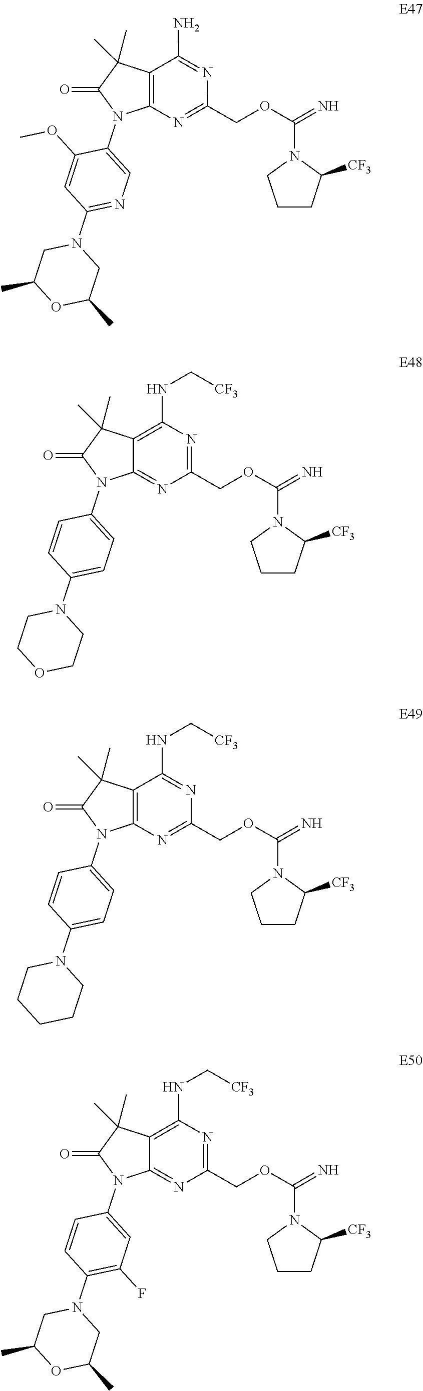 [SMILES: COc1cc(N2C[C@@H](C)O[C@@H](C)C2)ncc1N1C(=O)C(C)(C)c2c(N)nc(COC(=N)N3CCC[C@@H]3C(F)(F)F)nc21 ; CC1(C)C(=O)N(c2ccc(N3CCOCC3)cc2)c2nc(COC(=N)N3CCC[C@@H]3C(F)(F)F)nc(NCC(F)(F)F)c21 ; CC1(C)C(=O)N(c2ccc(N3CCCCC3)cc2)c2nc(COC(=N)N3CCC[C@@H]3C(F)(F)F)nc(NCC(F)(F)F)c21 ; C[C@@H]1CN(c2ccc(N3C(=O)C(C)(C)c4c(NCC(F)(F)F)nc(COC(=N)N5CCC[C@@H]5C(F)(F)F)nc43)cc2F)C[C@H](C)O1]

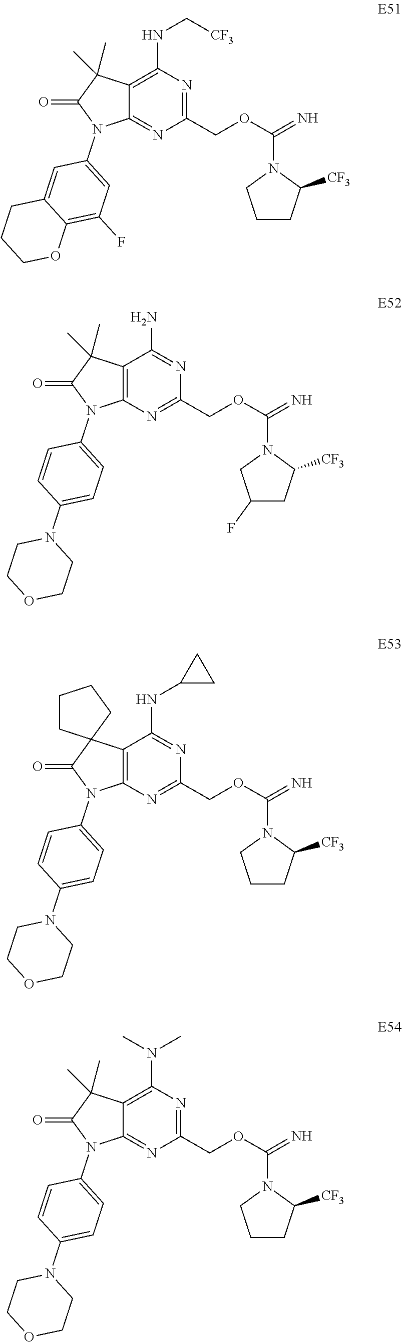 [SMILES: CC1(C)C(=O)N(c2cc(F)c3c(c2)CCCO3)c2nc(COC(=N)N3CCC[C@@H]3C(F)(F)F)nc(NCC(F)(F)F)c21 ; CC1(C)C(=O)N(c2ccc(N3CCOCC3)cc2)c2nc(COC(=N)N3CC(F)C[C@H]3C(F)(F)F)nc(N)c21 ; N=C(OCc1nc(NC2CC2)c2c(n1)N(c1ccc(N3CCOCC3)cc1)C(=O)C21CCCC1)N1CCC[C@@H]1C(F)(F)F ; CN(C)c1nc(COC(=N)N2CCC[C@@H]2C(F)(F)F)nc2c1C(C)(C)C(=O)N2c1ccc(N2CCOCC2)cc1]

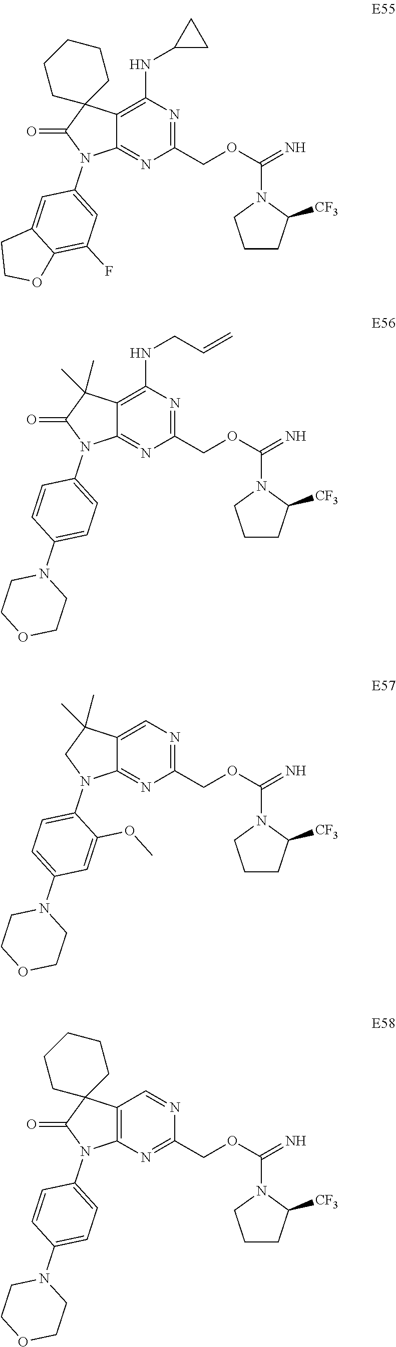 [SMILES: N=C(OCc1nc(NC2CC2)c2c(n1)N(c1cc(F)c3c(c1)CCO3)C(=O)C21CCCCC1)N1CCC[C@@H]1C(F)(F)F ; C=CCNc1nc(COC(=N)N2CCC[C@@H]2C(F)(F)F)nc2c1C(C)(C)C(=O)N2c1ccc(N2CCOCC2)cc1 ; COc1cc(N2CCOCC2)ccc1N1CC(C)(C)c2cnc(COC(=N)N3CCC[C@@H]3C(F)(F)F)nc21 ; N=C(OCc1ncc2c(n1)N(c1ccc(N3CCOCC3)cc1)C(=O)C21CCCCC1)N1CCC[C@@H]1C(F)(F)F]

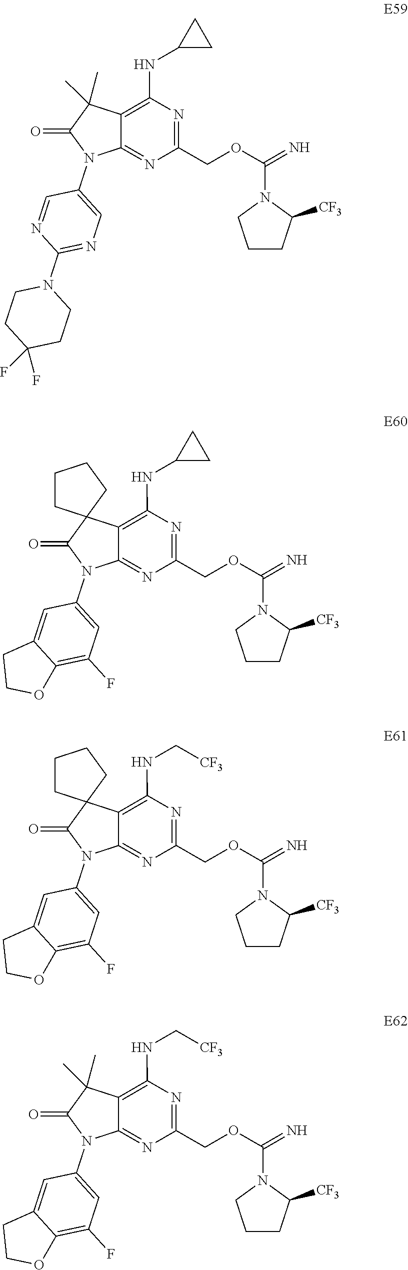 [SMILES: CC1(C)C(=O)N(c2cnc(N3CCC(F)(F)CC3)nc2)c2nc(COC(=N)N3CCC[C@@H]3C(F)(F)F)nc(NC3CC3)c21 ; N=C(OCc1nc(NC2CC2)c2c(n1)N(c1cc(F)c3c(c1)CCO3)C(=O)C21CCCC1)N1CCC[C@@H]1C(F)(F)F ; N=C(OCc1nc(NCC(F)(F)F)c2c(n1)N(c1cc(F)c3c(c1)CCO3)C(=O)C21CCCC1)N1CCC[C@@H]1C(F)(F)F ; CC1(C)C(=O)N(c2cc(F)c3c(c2)CCO3)c2nc(COC(=N)N3CCC[C@@H]3C(F)(F)F)nc(NCC(F)(F)F)c21]

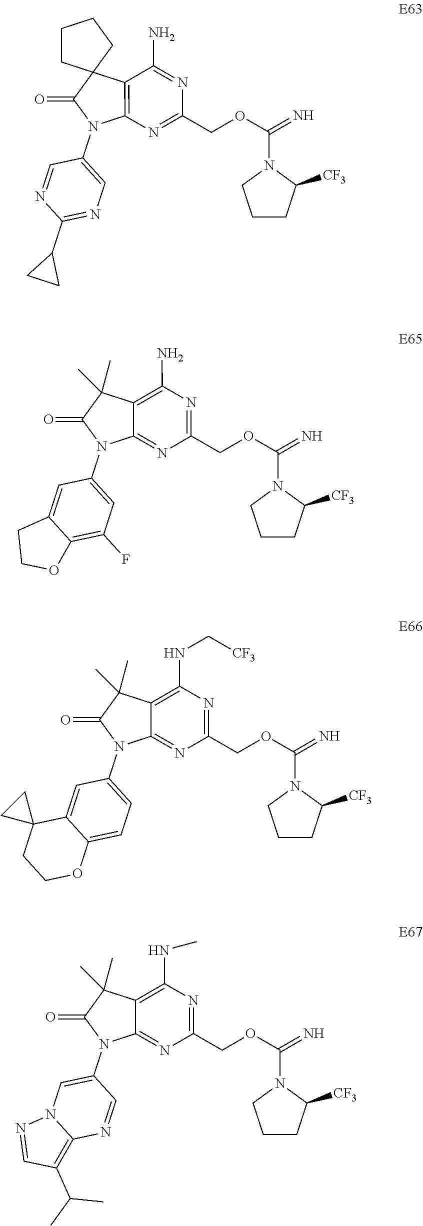 [SMILES: N=C(OCc1nc(N)c2c(n1)N(c1cnc(C3CC3)nc1)C(=O)C21CCCC1)N1CCC[C@@H]1C(F)(F)F ; CC1(C)C(=O)N(c2cc(F)c3c(c2)CCO3)c2nc(COC(=N)N3CCC[C@@H]3C(F)(F)F)nc(N)c21 ; CC1(C)C(=O)N(c2ccc3c(c2)C2(CCO3)CC2)c2nc(COC(=N)N3CCC[C@@H]3C(F)(F)F)nc(NCC(F)(F)F)c21 ; CNc1nc(COC(=N)N2CCC[C@@H]2C(F)(F)F)nc2c1C(C)(C)C(=O)N2c1cnc2c(C(C)C)cnn2c1]

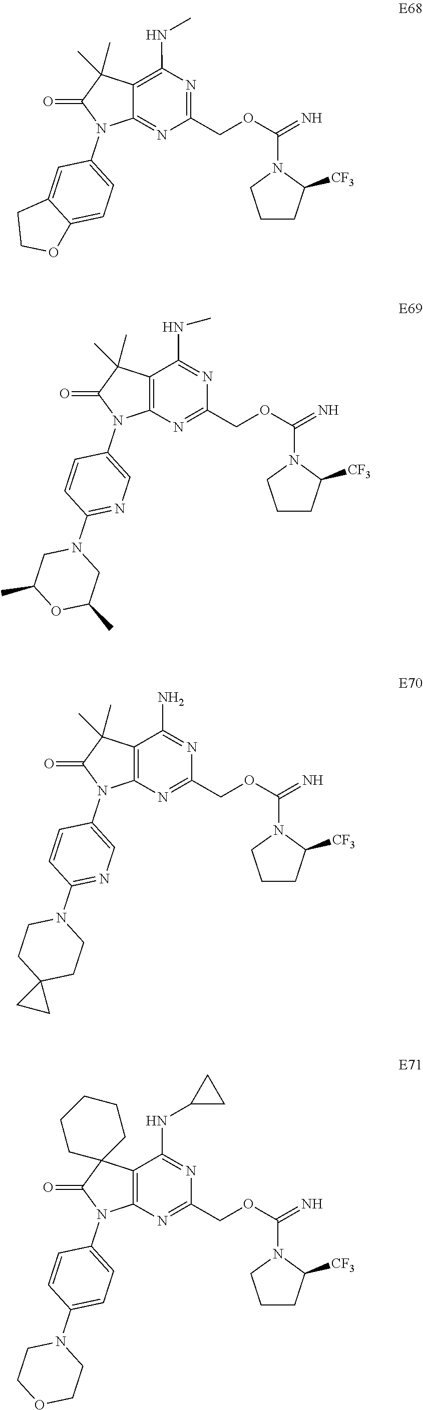 [SMILES: CNc1nc(COC(=N)N2CCC[C@@H]2C(F)(F)F)nc2c1C(C)(C)C(=O)N2c1ccc2c(c1)CCO2 ; CNc1nc(COC(=N)N2CCC[C@@H]2C(F)(F)F)nc2c1C(C)(C)C(=O)N2c1ccc(N2C[C@@H](C)O[C@@H](C)C2)nc1 ; CC1(C)C(=O)N(c2ccc(N3CCC4(CC3)CC4)nc2)c2nc(COC(=N)N3CCC[C@@H]3C(F)(F)F)nc(N)c21 ; N=C(OCc1nc(NC2CC2)c2c(n1)N(c1ccc(N3CCOCC3)cc1)C(=O)C21CCCCC1)N1CCC[C@@H]1C(F)(F)F]

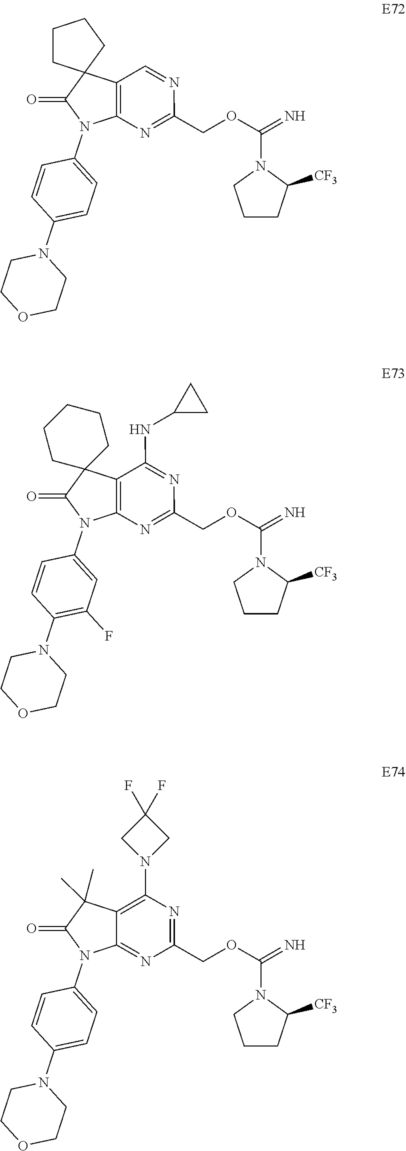 [SMILES: N=C(OCc1ncc2c(n1)N(c1ccc(N3CCOCC3)cc1)C(=O)C21CCCC1)N1CCC[C@@H]1C(F)(F)F ; N=C(OCc1nc(NC2CC2)c2c(n1)N(c1ccc(N3CCOCC3)c(F)c1)C(=O)C21CCCCC1)N1CCC[C@@H]1C(F)(F)F ; CC1(C)C(=O)N(c2ccc(N3CCOCC3)cc2)c2nc(COC(=N)N3CCC[C@@H]3C(F)(F)F)nc(N3CC(F)(F)C3)c21]

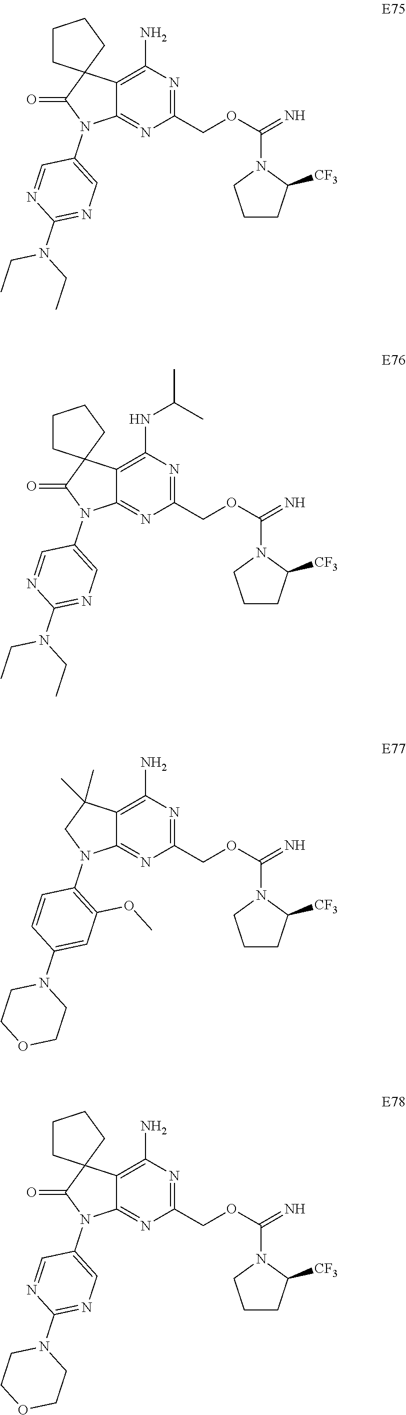 [SMILES: CCN(CC)c1ncc(N2C(=O)C3(CCCC3)c3c(N)nc(COC(=N)N4CCC[C@@H]4C(F)(F)F)nc32)cn1 ; CCN(CC)c1ncc(N2C(=O)C3(CCCC3)c3c(NC(C)C)nc(COC(=N)N4CCC[C@@H]4C(F)(F)F)nc32)cn1 ; COc1cc(N2CCOCC2)ccc1N1CC(C)(C)c2c(N)nc(COC(=N)N3CCC[C@@H]3C(F)(F)F)nc21 ; N=C(OCc1nc(N)c2c(n1)N(c1cnc(N3CCOCC3)nc1)C(=O)C21CCCC1)N1CCC[C@@H]1C(F)(F)F]

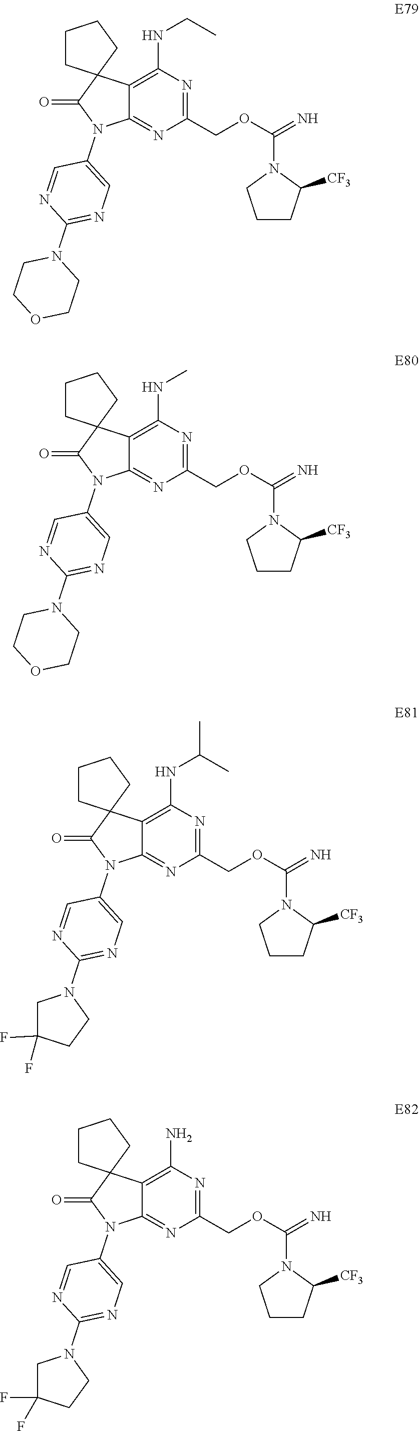 [SMILES: CCNc1nc(COC(=N)N2CCC[C@@H]2C(F)(F)F)nc2c1C1(CCCC1)C(=O)N2c1cnc(N2CCOCC2)nc1 ; CNc1nc(COC(=N)N2CCC[C@@H]2C(F)(F)F)nc2c1C1(CCCC1)C(=O)N2c1cnc(N2CCOCC2)nc1 ; CC(C)Nc1nc(COC(=N)N2CCC[C@@H]2C(F)(F)F)nc2c1C1(CCCC1)C(=O)N2c1cnc(N2CCC(F)(F)C2)nc1 ; N=C(OCc1nc(N)c2c(n1)N(c1cnc(N3CCC(F)(F)C3)nc1)C(=O)C21CCCC1)N1CCC[C@@H]1C(F)(F)F]

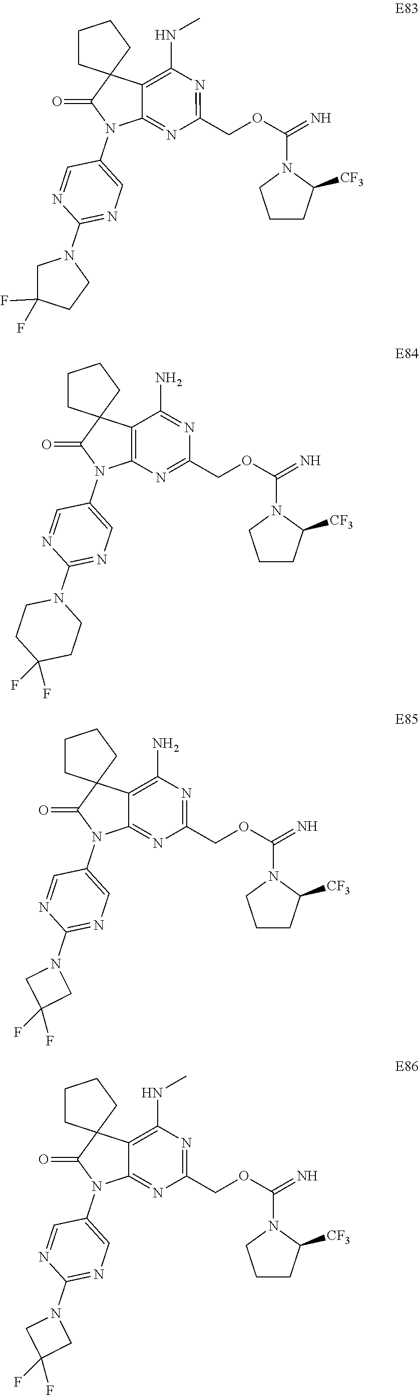 [SMILES: CNc1nc(COC(=N)N2CCC[C@@H]2C(F)(F)F)nc2c1C1(CCCC1)C(=O)N2c1cnc(N2CCC(F)(F)C2)nc1 ; N=C(OCc1nc(N)c2c(n1)N(c1cnc(N3CCC(F)(F)CC3)nc1)C(=O)C21CCCC1)N1CCC[C@@H]1C(F)(F)F ; N=C(OCc1nc(N)c2c(n1)N(c1cnc(N3CC(F)(F)C3)nc1)C(=O)C21CCCC1)N1CCC[C@@H]1C(F)(F)F ; CNc1nc(COC(=N)N2CCC[C@@H]2C(F)(F)F)nc2c1C1(CCCC1)C(=O)N2c1cnc(N2CC(F)(F)C2)nc1]

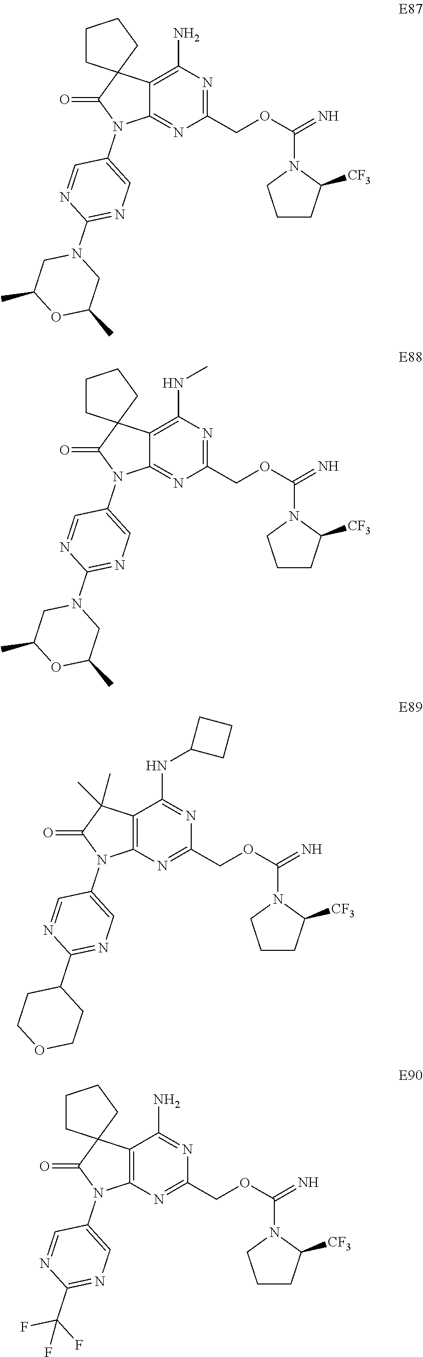

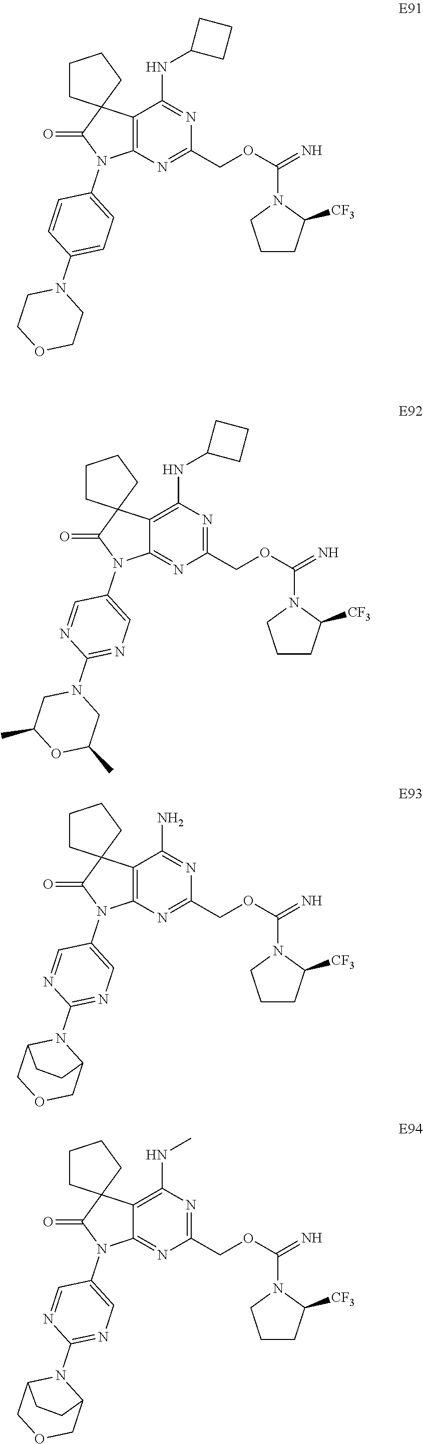

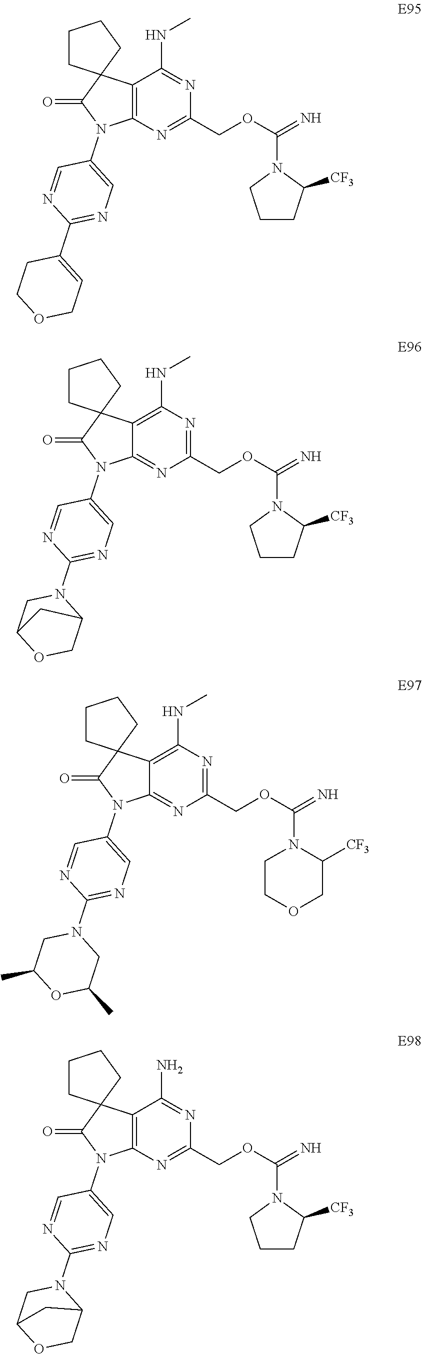

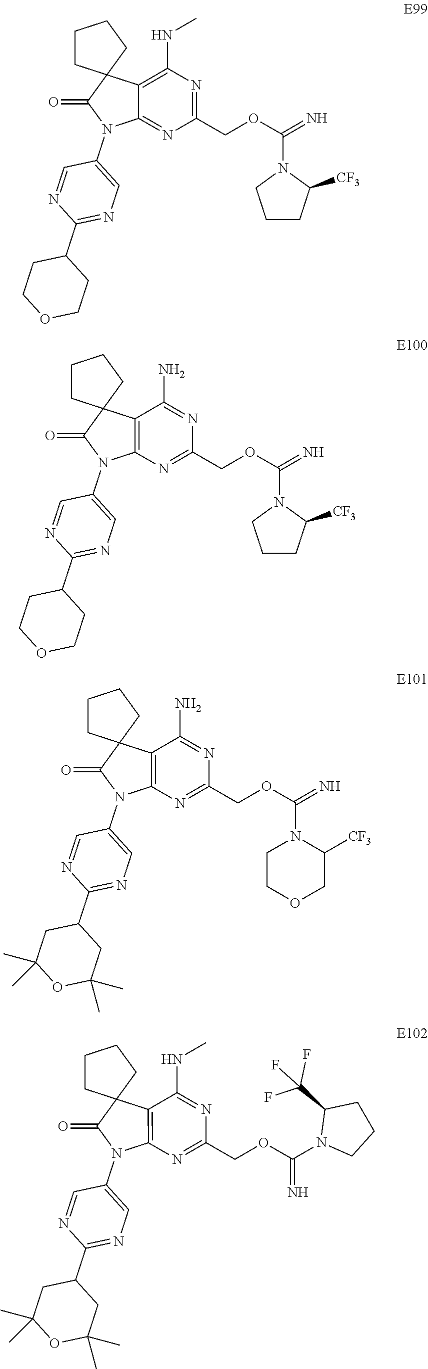

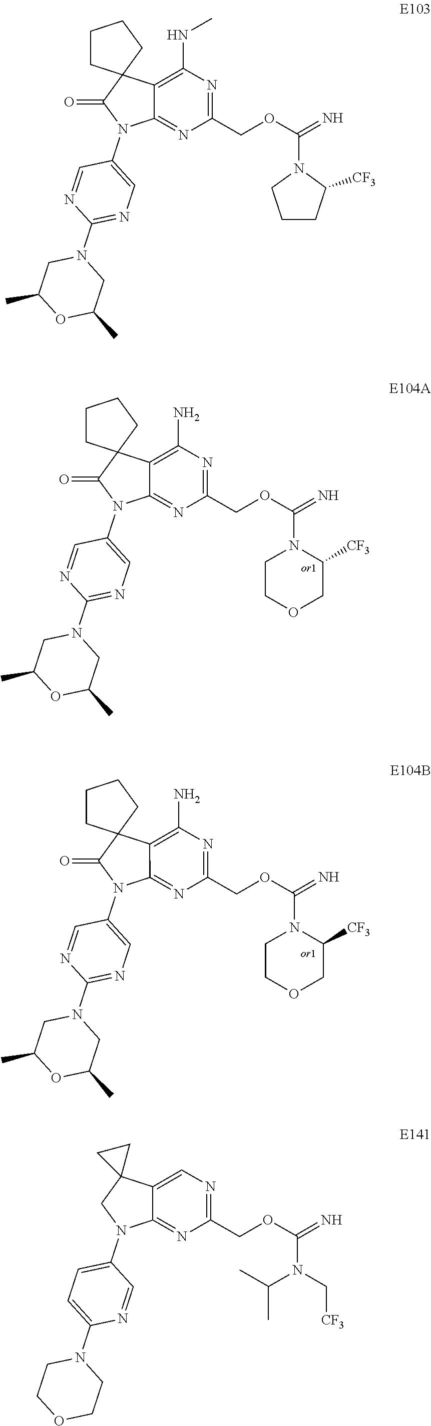 [SMILES: CNc1nc(COC(=N)N2CCC[C@H]2C(F)(F)F)nc2c1C1(CCCC1)C(=O)N2c1cnc(N2C[C@@H](C)O[C@@H](C)C2)nc1 ; C[C@@H]1CN(c2ncc(N3C(=O)C4(CCCC4)c4c(N)nc(COC(=N)N5CCOC[C@H]5C(F)(F)F)nc43)cn2)C[C@H](C)O1 ; C[C@@H]1CN(c2ncc(N3C(=O)C4(CCCC4)c4c(N)nc(COC(=N)N5CCOC[C@@H]5C(F)(F)F)nc43)cn2)C[C@H](C)O1 ; CC(C)N(CC(F)(F)F)C(=N)OCc1ncc2c(n1)N(c1ccc(N3CCOCC3)nc1)CC21CC1]

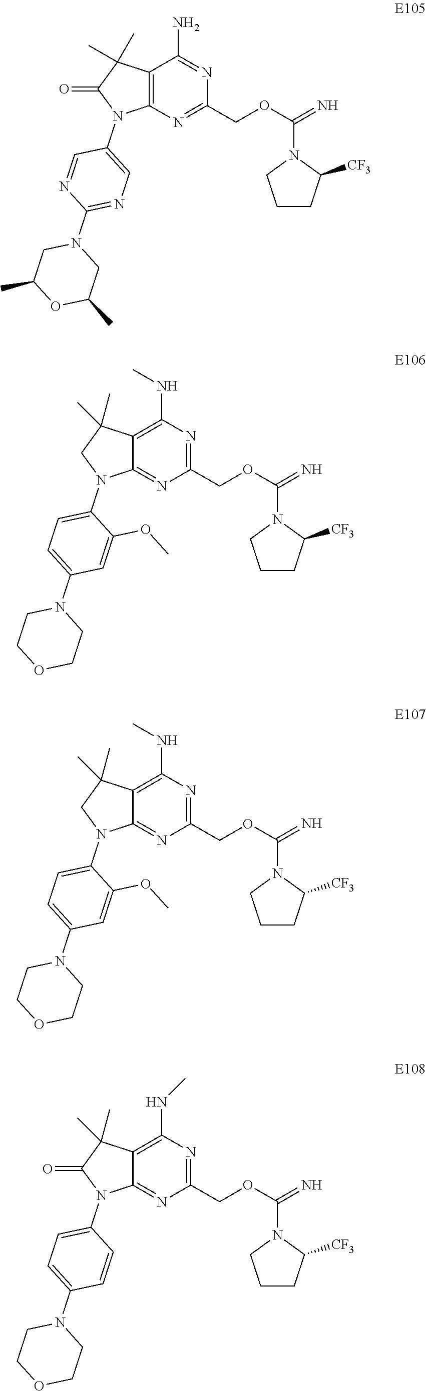 [SMILES: C[C@@H]1CN(c2ncc(N3C(=O)C(C)(C)c4c(N)nc(COC(=N)N5CCC[C@@H]5C(F)(F)F)nc43)cn2)C[C@H](C)O1 ; CNc1nc(COC(=N)N2CCC[C@@H]2C(F)(F)F)nc2c1C(C)(C)CN2c1ccc(N2CCOCC2)cc1OC ; CNc1nc(COC(=N)N2CCC[C@H]2C(F)(F)F)nc2c1C(C)(C)CN2c1ccc(N2CCOCC2)cc1OC ; CNc1nc(COC(=N)N2CCC[C@H]2C(F)(F)F)nc2c1C(C)(C)C(=O)N2c1ccc(N2CCOCC2)cc1]

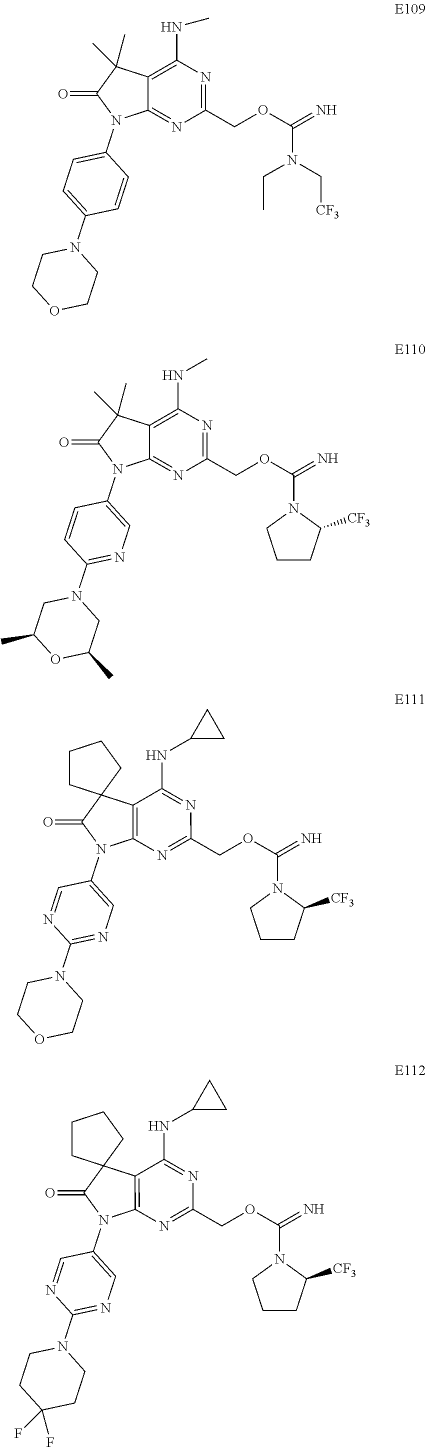 [SMILES: CCN(CC(F)(F)F)C(=N)OCc1nc(NC)c2c(n1)N(c1ccc(N3CCOCC3)cc1)C(=O)C2(C)C ; CNc1nc(COC(=N)N2CCC[C@H]2C(F)(F)F)nc2c1C(C)(C)C(=O)N2c1ccc(N2C[C@@H](C)O[C@@H](C)C2)nc1 ; N=C(OCc1nc(NC2CC2)c2c(n1)N(c1cnc(N3CCOCC3)nc1)C(=O)C21CCCC1)N1CCC[C@@H]1C(F)(F)F ; N=C(OCc1nc(NC2CC2)c2c(n1)N(c1cnc(N3CCC(F)(F)CC3)nc1)C(=O)C21CCCC1)N1CCC[C@@H]1C(F)(F)F]

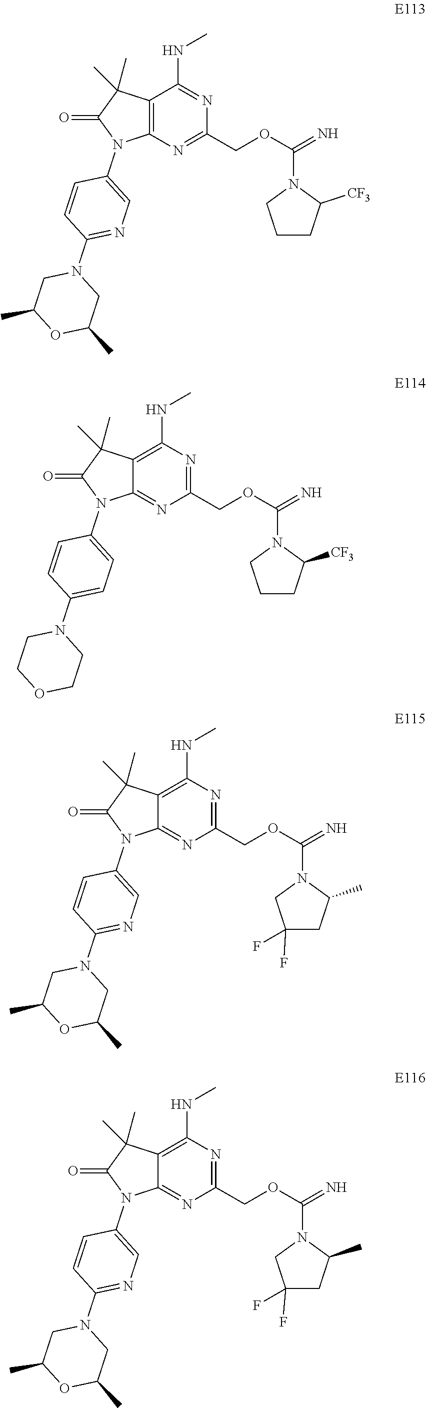

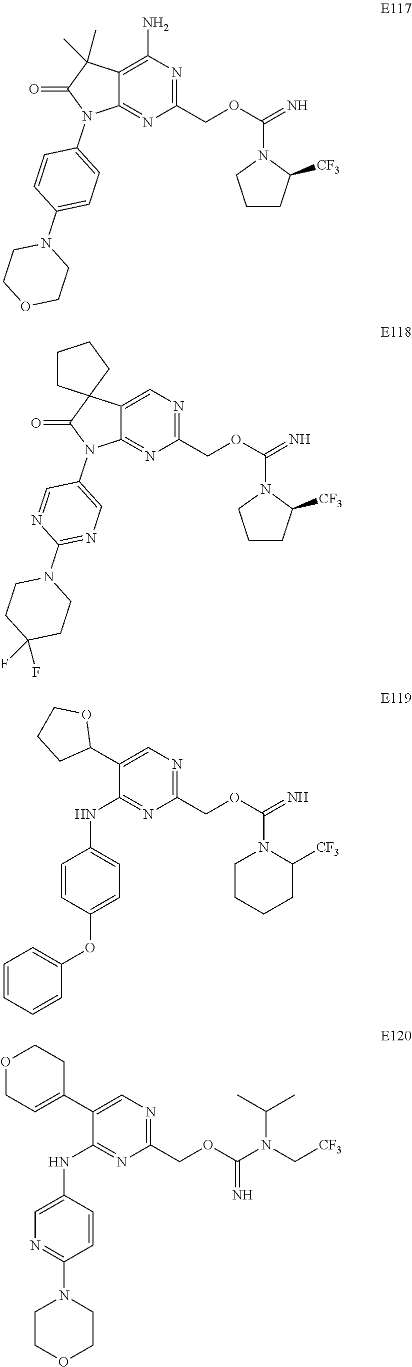

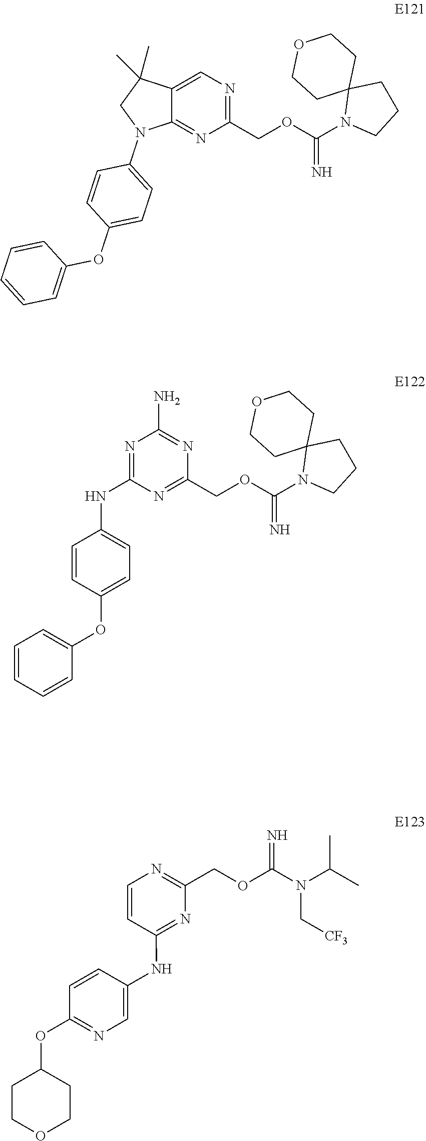 [SMILES: CC1(C)CN(c2ccc(Oc3ccccc3)cc2)c2nc(COC(=N)N3CCCC34CCOCC4)ncc21 ; N=C(OCc1nc(N)nc(Nc2ccc(Oc3ccccc3)cc2)n1)N1CCCC12CCOCC2 ; CC(C)N(CC(F)(F)F)C(=N)OCc1nccc(Nc2ccc(OC3CCOCC3)nc2)n1]

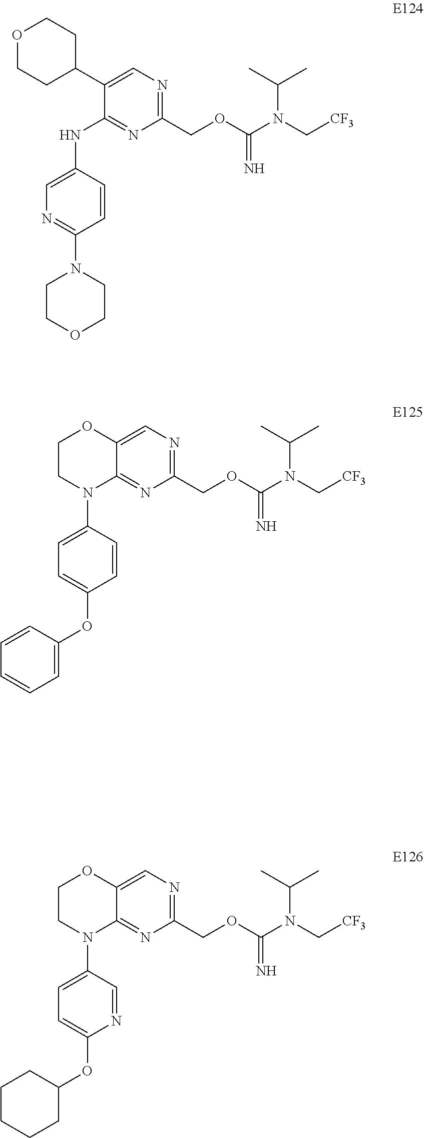 [SMILES: CC(C)N(CC(F)(F)F)C(=N)OCc1ncc(C2CCOCC2)c(Nc2ccc(N3CCOCC3)nc2)n1 ; CC(C)N(CC(F)(F)F)C(=N)OCc1ncc2c(n1)N(c1ccc(Oc3ccccc3)cc1)CCO2 ; CC(C)N(CC(F)(F)F)C(=N)OCc1ncc2c(n1)N(c1ccc(OC3CCCCC3)nc1)CCO2]

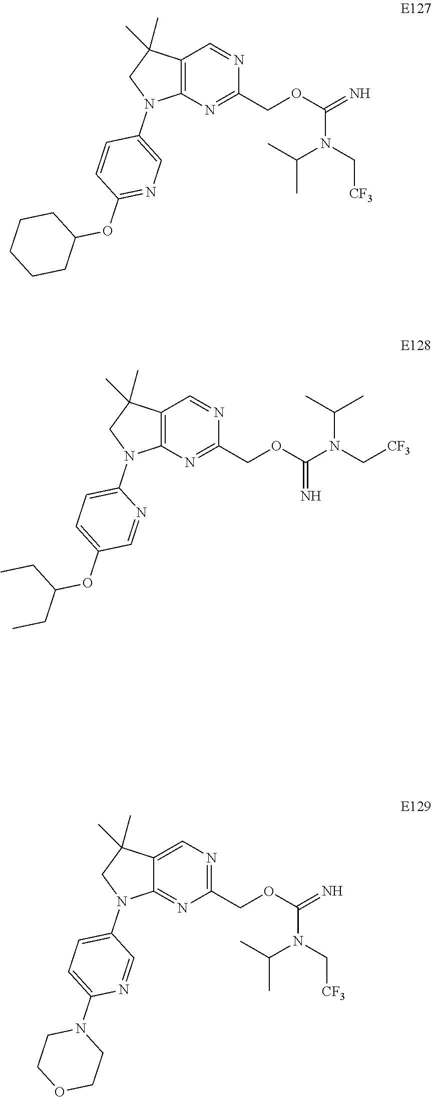 [SMILES: CC(C)N(CC(F)(F)F)C(=N)OCc1ncc2c(n1)N(c1ccc(OC3CCCCC3)nc1)CC2(C)C ; CCC(CC)Oc1ccc(N2CC(C)(C)c3cnc(COC(=N)N(CC(F)(F)F)C(C)C)nc32)nc1 ; CC(C)N(CC(F)(F)F)C(=N)OCc1ncc2c(n1)N(c1ccc(N3CCOCC3)nc1)CC2(C)C]

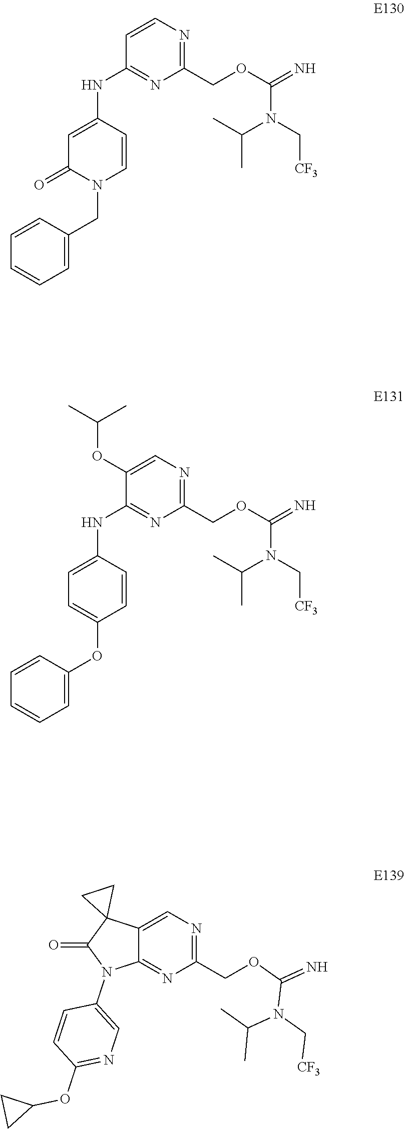 [SMILES: CC(C)N(CC(F)(F)F)C(=N)OCc1nccc(Nc2ccn(Cc3ccccc3)c(=O)c2)n1 ; CC(C)Oc1cnc(COC(=N)N(CC(F)(F)F)C(C)C)nc1Nc1ccc(Oc2ccccc2)cc1 ; CC(C)N(CC(F)(F)F)C(=N)OCc1ncc2c(n1)N(c1ccc(OC3CC3)nc1)C(=O)C21CC1]

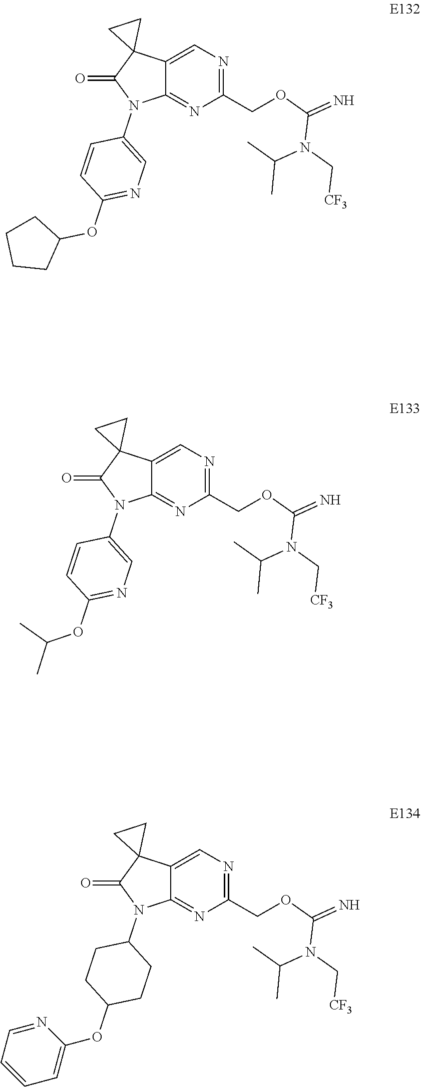 [SMILES: CC(C)N(CC(F)(F)F)C(=N)OCc1ncc2c(n1)N(c1ccc(OC3CCCC3)nc1)C(=O)C21CC1 ; CC(C)Oc1ccc(N2C(=O)C3(CC3)c3cnc(COC(=N)N(CC(F)(F)F)C(C)C)nc32)cn1 ; CC(C)N(CC(F)(F)F)C(=N)OCc1ncc2c(n1)N(C1CCC(Oc3ccccn3)CC1)C(=O)C21CC1]

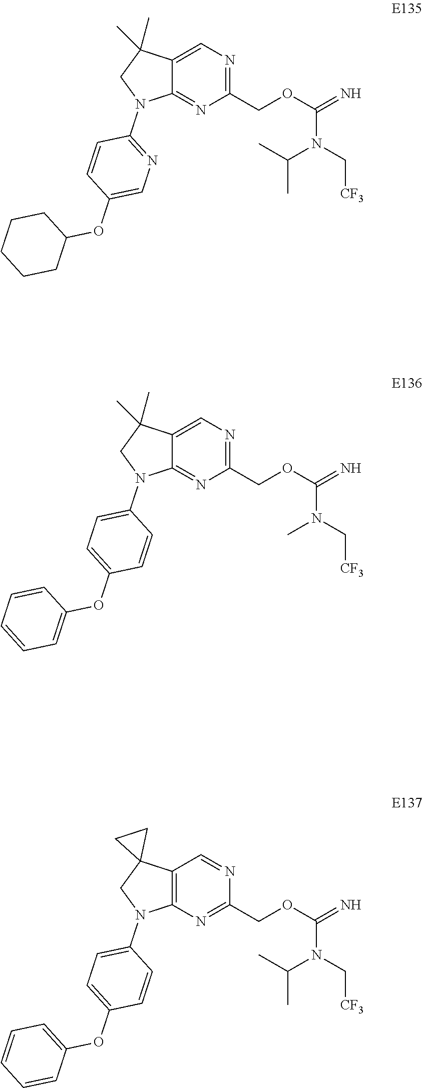 [SMILES: CC(C)N(CC(F)(F)F)C(=N)OCc1ncc2c(n1)N(c1ccc(OC3CCCCC3)cn1)CC2(C)C ; CN(CC(F)(F)F)C(=N)OCc1ncc2c(n1)N(c1ccc(Oc3ccccc3)cc1)CC2(C)C ; CC(C)N(CC(F)(F)F)C(=N)OCc1ncc2c(n1)N(c1ccc(Oc3ccccc3)cc1)CC21CC1]

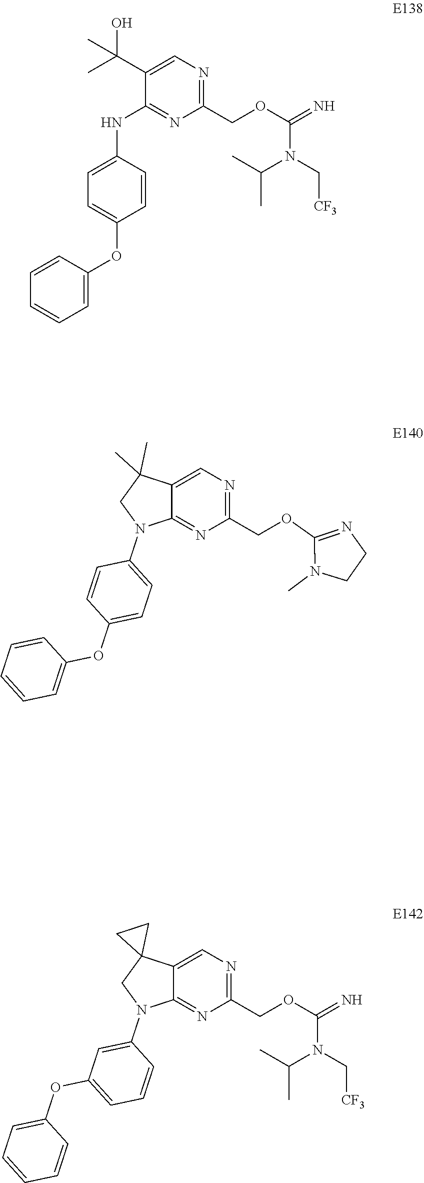 [SMILES: CC(C)N(CC(F)(F)F)C(=N)OCc1ncc(C(C)(C)O)c(Nc2ccc(Oc3ccccc3)cc2)n1 ; CN1CCN=C1OCc1ncc2c(n1)N(c1ccc(Oc3ccccc3)cc1)CC2(C)C ; CC(C)N(CC(F)(F)F)C(=N)OCc1ncc2c(n1)N(c1cccc(Oc3ccccc3)c1)CC21CC1]

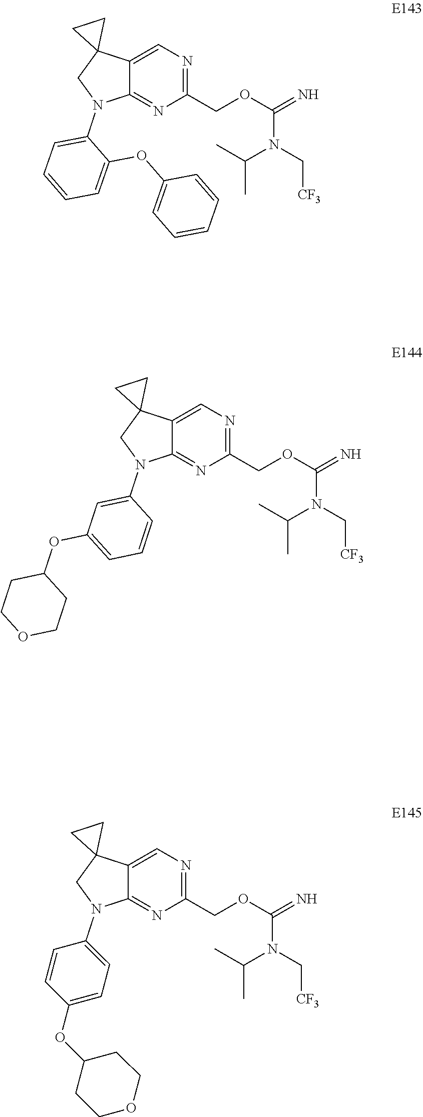 [SMILES: CC(C)N(CC(F)(F)F)C(=N)OCc1ncc2c(n1)N(c1ccccc1Oc1ccccc1)CC21CC1 ; CC(C)N(CC(F)(F)F)C(=N)OCc1ncc2c(n1)N(c1cccc(OC3CCOCC3)c1)CC21CC1 ; CC(C)N(CC(F)(F)F)C(=N)OCc1ncc2c(n1)N(c1ccc(OC3CCOCC3)cc1)CC21CC1]

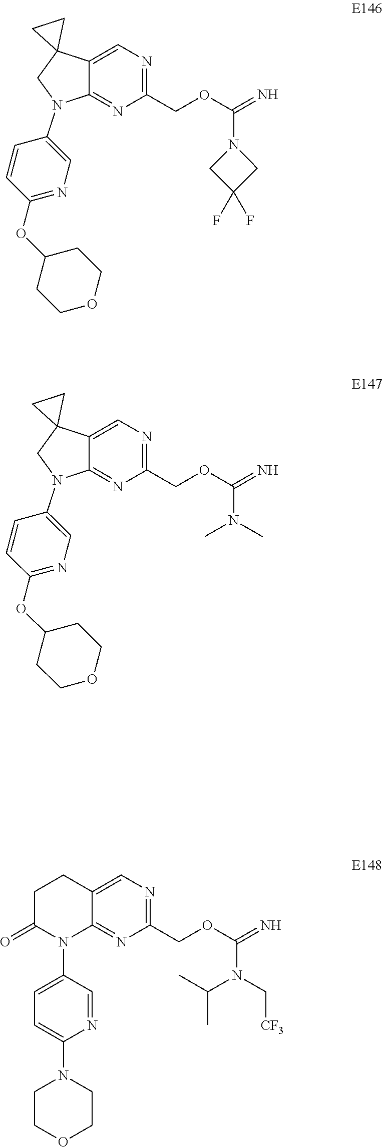 [SMILES: N=C(OCc1ncc2c(n1)N(c1ccc(OC3CCOCC3)nc1)CC21CC1)N1CC(F)(F)C1 ; CN(C)C(=N)OCc1ncc2c(n1)N(c1ccc(OC3CCOCC3)nc1)CC21CC1 ; CC(C)N(CC(F)(F)F)C(=N)OCc1ncc2c(n1)N(c1ccc(N3CCOCC3)nc1)C(=O)CC2]

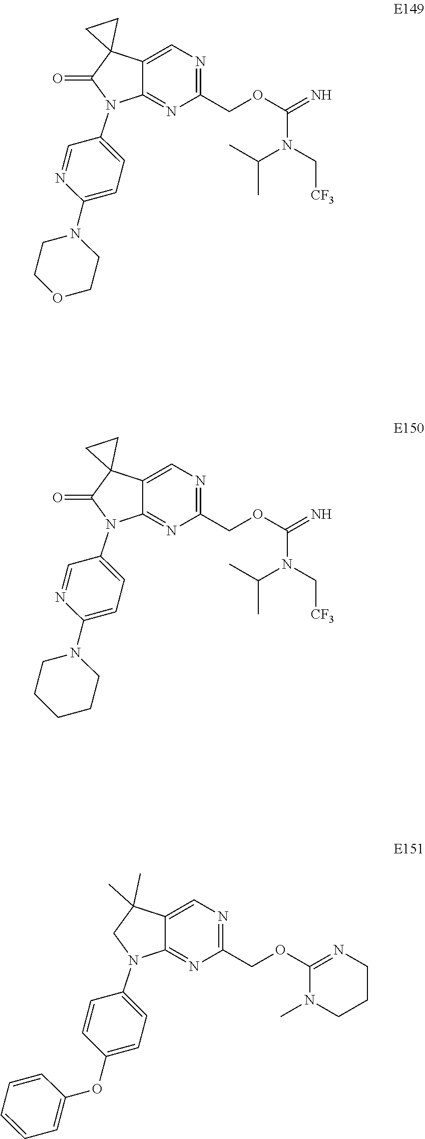 [SMILES: CC(C)N(CC(F)(F)F)C(=N)OCc1ncc2c(n1)N(c1ccc(N3CCOCC3)nc1)C(=O)C21CC1 ; CC(C)N(CC(F)(F)F)C(=N)OCc1ncc2c(n1)N(c1ccc(N3CCCCC3)nc1)C(=O)C21CC1 ; CN1CCCN=C1OCc1ncc2c(n1)N(c1ccc(Oc3ccccc3)cc1)CC2(C)C]

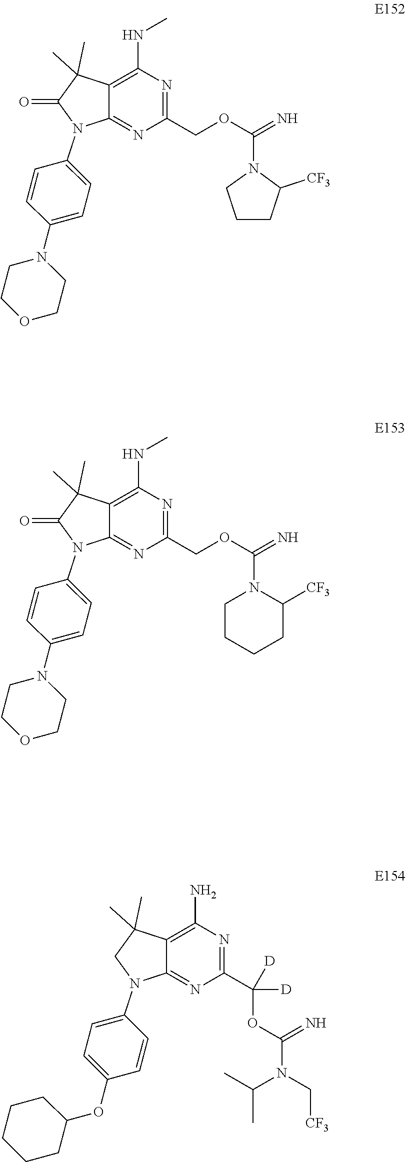 [SMILES: CNc1nc(COC(=N)N2CCCC2C(F)(F)F)nc2c1C(C)(C)C(=O)N2c1ccc(N2CCOCC2)cc1 ; CNc1nc(COC(=N)N2CCCCC2C(F)(F)F)nc2c1C(C)(C)C(=O)N2c1ccc(N2CCOCC2)cc1 ; [2H]C([2H])(OC(=N)N(CC(F)(F)F)C(C)C)c1nc(N)c2c(n1)N(c1ccc(OC3CCCCC3)cc1)CC2(C)C]

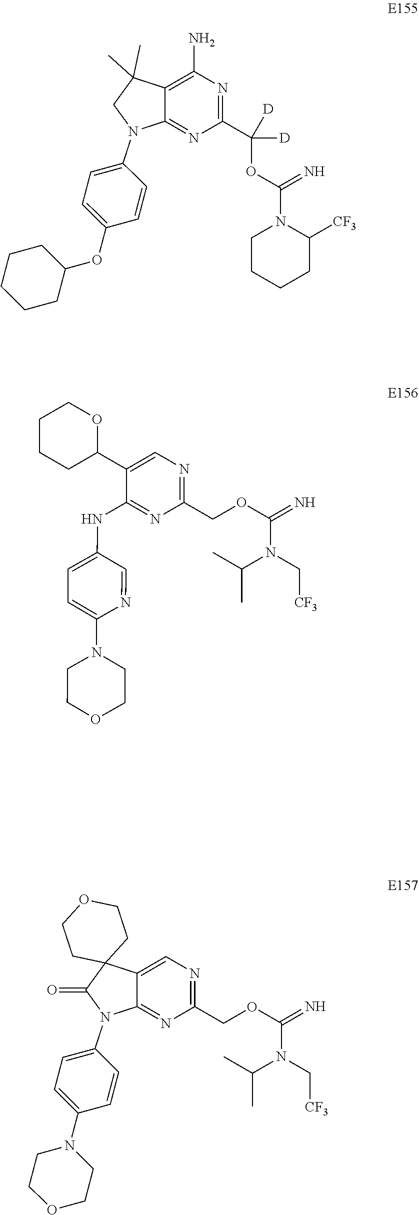 [SMILES: [2H]C([2H])(OC(=N)N1CCCCC1C(F)(F)F)c1nc(N)c2c(n1)N(c1ccc(OC3CCCCC3)cc1)CC2(C)C ; CC(C)N(CC(F)(F)F)C(=N)OCc1ncc(C2CCCCO2)c(Nc2ccc(N3CCOCC3)nc2)n1 ; CC(C)N(CC(F)(F)F)C(=N)OCc1ncc2c(n1)N(c1ccc(N3CCOCC3)cc1)C(=O)C21CCOCC1]

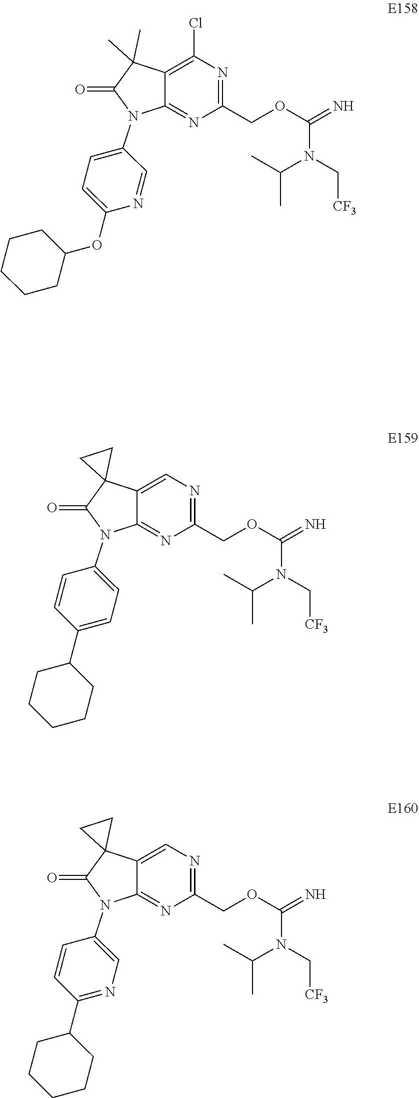 [SMILES: CC(C)N(CC(F)(F)F)C(=N)OCc1nc(Cl)c2c(n1)N(c1ccc(OC3CCCCC3)nc1)C(=O)C2(C)C ; CC(C)N(CC(F)(F)F)C(=N)OCc1ncc2c(n1)N(c1ccc(C3CCCCC3)cc1)C(=O)C21CC1 ; CC(C)N(CC(F)(F)F)C(=N)OCc1ncc2c(n1)N(c1ccc(C3CCCCC3)nc1)C(=O)C21CC1]

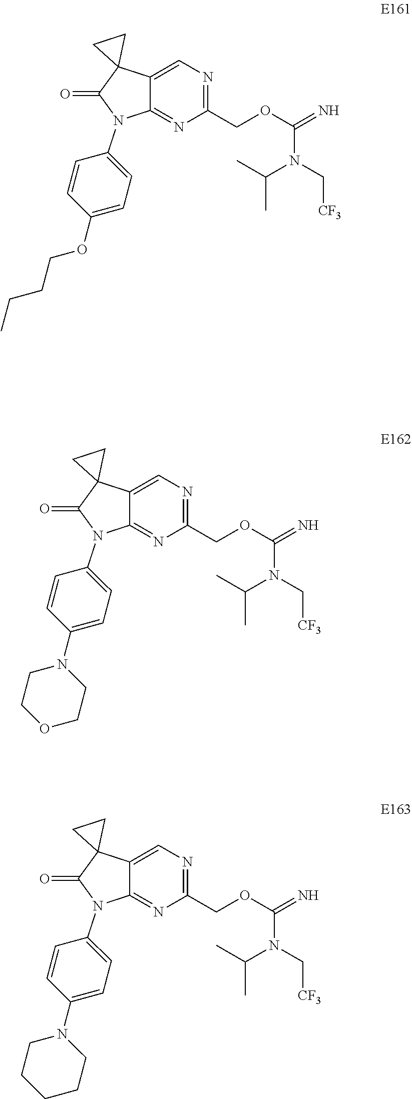 [SMILES: CCCCOc1ccc(N2C(=O)C3(CC3)c3cnc(COC(=N)N(CC(F)(F)F)C(C)C)nc32)cc1 ; CC(C)N(CC(F)(F)F)C(=N)OCc1ncc2c(n1)N(c1ccc(N3CCOCC3)cc1)C(=O)C21CC1 ; CC(C)N(CC(F)(F)F)C(=N)OCc1ncc2c(n1)N(c1ccc(N3CCCCC3)cc1)C(=O)C21CC1]

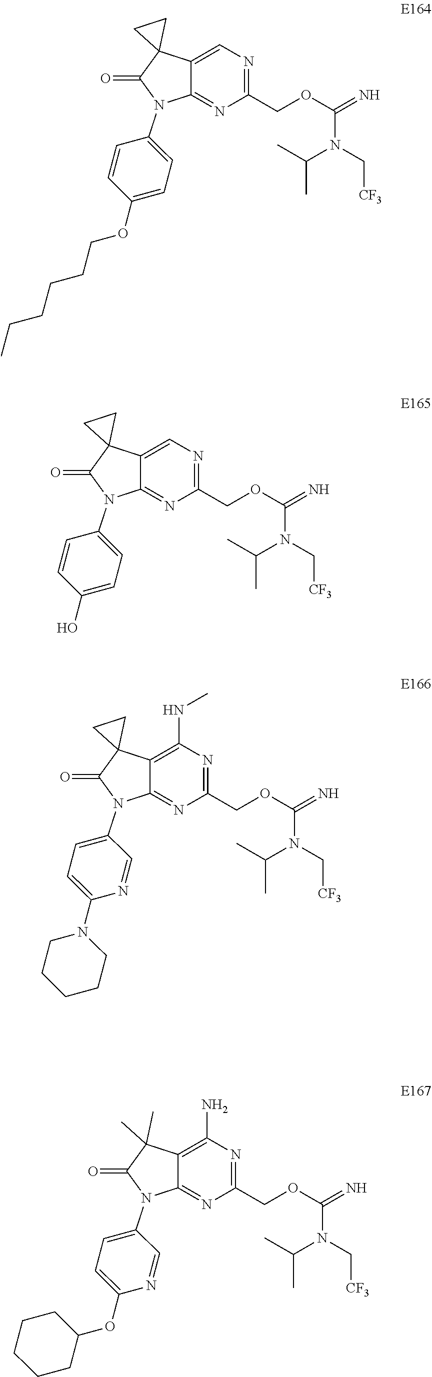 [SMILES: CCCCCCOc1ccc(N2C(=O)C3(CC3)c3cnc(COC(=N)N(CC(F)(F)F)C(C)C)nc32)cc1 ; CC(C)N(CC(F)(F)F)C(=N)OCc1ncc2c(n1)N(c1ccc(O)cc1)C(=O)C21CC1 ; CNc1nc(COC(=N)N(CC(F)(F)F)C(C)C)nc2c1C1(CC1)C(=O)N2c1ccc(N2CCCCC2)nc1 ; CC(C)N(CC(F)(F)F)C(=N)OCc1nc(N)c2c(n1)N(c1ccc(OC3CCCCC3)nc1)C(=O)C2(C)C]

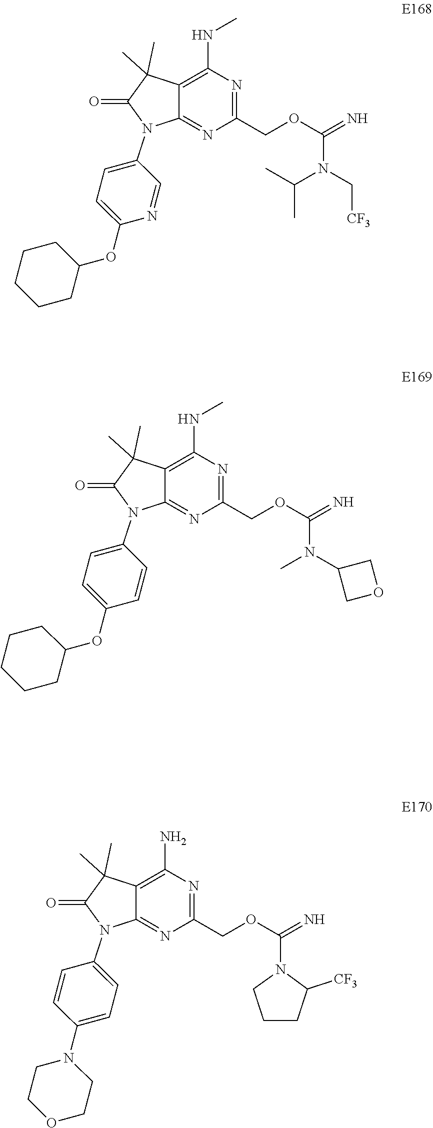 [SMILES: CNc1nc(COC(=N)N(CC(F)(F)F)C(C)C)nc2c1C(C)(C)C(=O)N2c1ccc(OC2CCCCC2)nc1 ; CNc1nc(COC(=N)N(C)C2COC2)nc2c1C(C)(C)C(=O)N2c1ccc(OC2CCCCC2)cc1 ; CC1(C)C(=O)N(c2ccc(N3CCOCC3)cc2)c2nc(COC(=N)N3CCCC3C(F)(F)F)nc(N)c21]

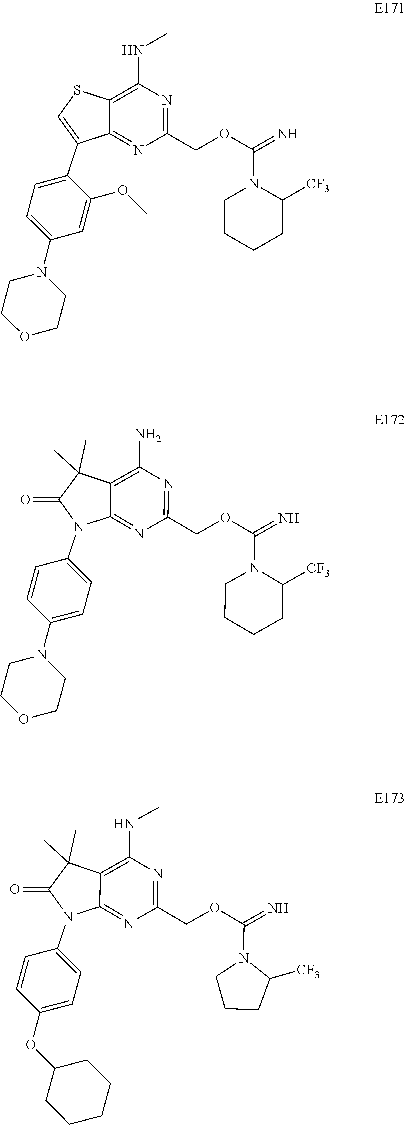 [SMILES: CNc1nc(COC(=N)N2CCCCC2C(F)(F)F)nc2c(-c3ccc(N4CCOCC4)cc3OC)csc12 ; CC1(C)C(=O)N(c2ccc(N3CCOCC3)cc2)c2nc(COC(=N)N3CCCCC3C(F)(F)F)nc(N)c21 ; CNc1nc(COC(=N)N2CCCC2C(F)(F)F)nc2c1C(C)(C)C(=O)N2c1ccc(OC2CCCCC2)cc1]

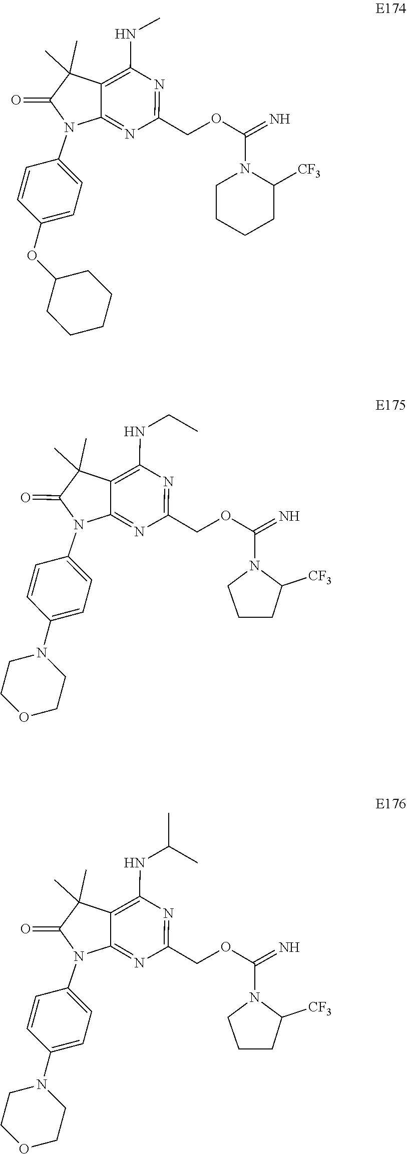 [SMILES: CNc1nc(COC(=N)N2CCCCC2C(F)(F)F)nc2c1C(C)(C)C(=O)N2c1ccc(OC2CCCCC2)cc1 ; CCNc1nc(COC(=N)N2CCCC2C(F)(F)F)nc2c1C(C)(C)C(=O)N2c1ccc(N2CCOCC2)cc1 ; CC(C)Nc1nc(COC(=N)N2CCCC2C(F)(F)F)nc2c1C(C)(C)C(=O)N2c1ccc(N2CCOCC2)cc1]

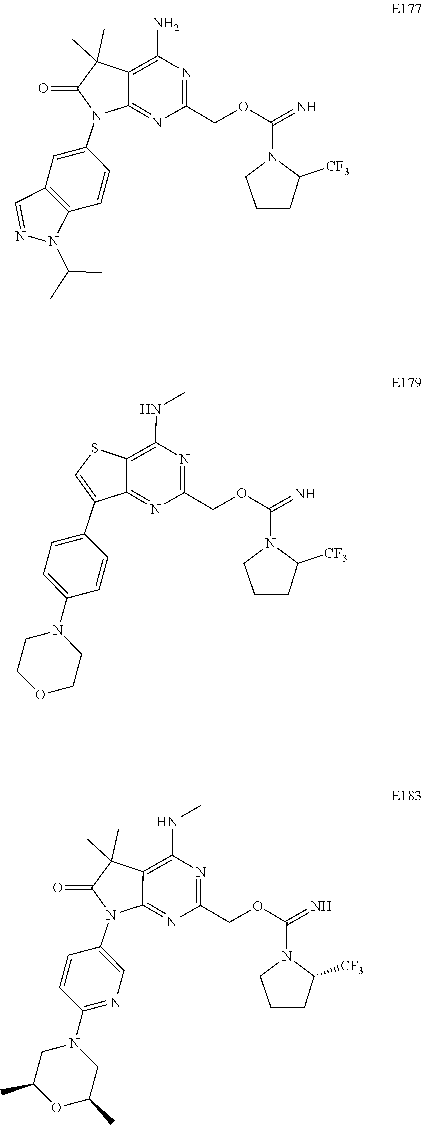 [SMILES: CC(C)n1ncc2cc(N3C(=O)C(C)(C)c4c(N)nc(COC(=N)N5CCCC5C(F)(F)F)nc43)ccc21 ; CNc1nc(COC(=N)N2CCCC2C(F)(F)F)nc2c(-c3ccc(N4CCOCC4)cc3)csc12 ; CNc1nc(COC(=N)N2CCC[C@H]2C(F)(F)F)nc2c1C(C)(C)C(=O)N2c1ccc(N2C[C@@H](C)O[C@@H](C)C2)nc1]

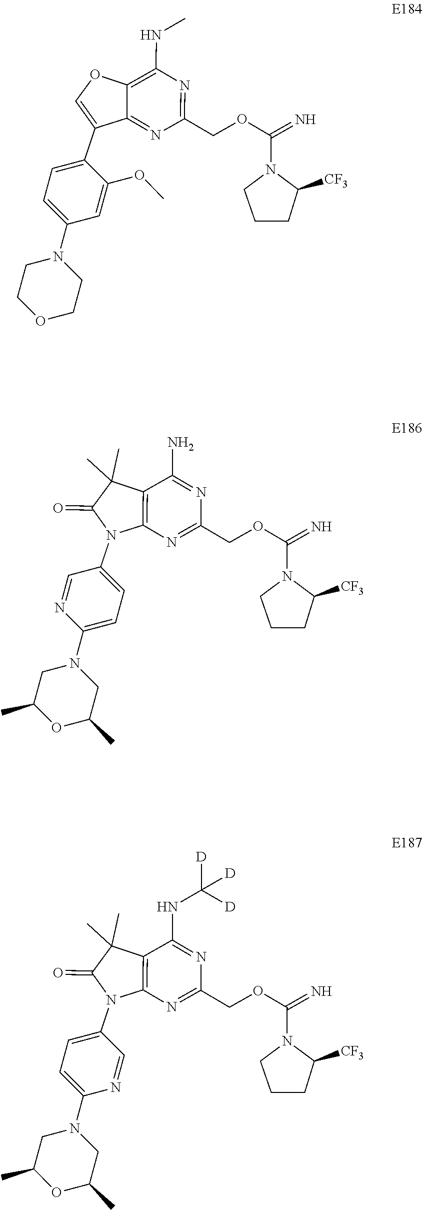 [SMILES: CNc1nc(COC(=N)N2CCC[C@@H]2C(F)(F)F)nc2c(-c3ccc(N4CCOCC4)cc3OC)coc12 ; C[C@@H]1CN(c2ccc(N3C(=O)C(C)(C)c4c(N)nc(COC(=N)N5CCC[C@@H]5C(F)(F)F)nc43)cn2)C[C@H](C)O1 ; [2H]C([2H])([2H])Nc1nc(COC(=N)N2CCC[C@@H]2C(F)(F)F)nc2c1C(C)(C)C(=O)N2c1ccc(N2C[C@@H](C)O[C@@H](C)C2)nc1]

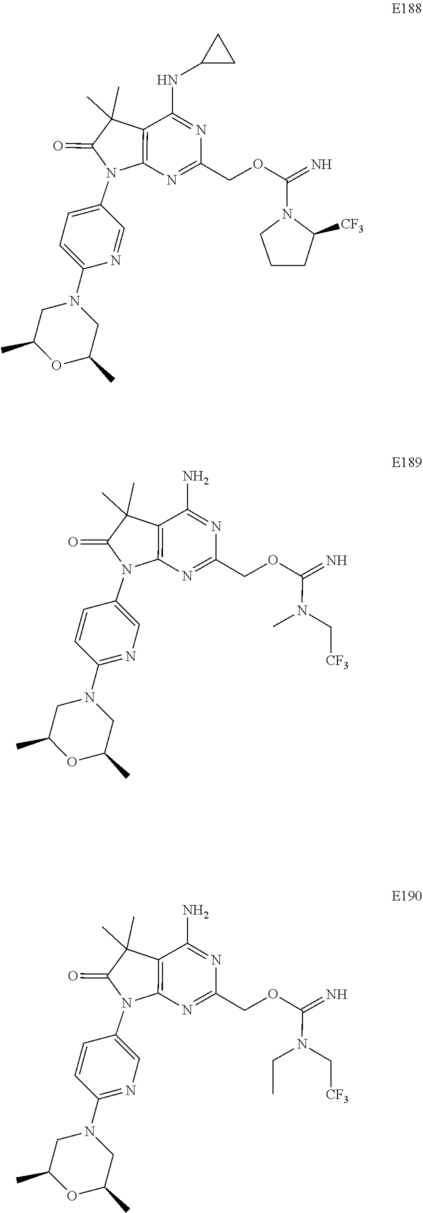 [SMILES: C[C@@H]1CN(c2ccc(N3C(=O)C(C)(C)c4c(NC5CC5)nc(COC(=N)N5CCC[C@@H]5C(F)(F)F)nc43)cn2)C[C@H](C)O1 ; C[C@@H]1CN(c2ccc(N3C(=O)C(C)(C)c4c(N)nc(COC(=N)N(C)CC(F)(F)F)nc43)cn2)C[C@H](C)O1 ; CCN(CC(F)(F)F)C(=N)OCc1nc(N)c2c(n1)N(c1ccc(N3C[C@@H](C)O[C@@H](C)C3)nc1)C(=O)C2(C)C]

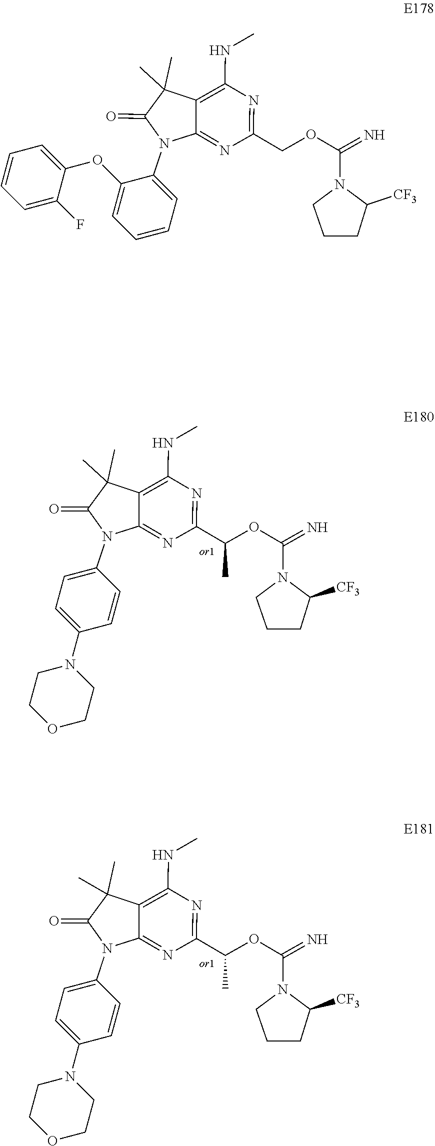 [SMILES: CNc1nc(COC(=N)N2CCCC2C(F)(F)F)nc2c1C(C)(C)C(=O)N2c1ccccc1Oc1ccccc1F ; CNc1nc([C@H](C)OC(=N)N2CCC[C@@H]2C(F)(F)F)nc2c1C(C)(C)C(=O)N2c1ccc(N2CCOCC2)cc1 ; CNc1nc([C@@H](C)OC(=N)N2CCC[C@@H]2C(F)(F)F)nc2c1C(C)(C)C(=O)N2c1ccc(N2CCOCC2)cc1]

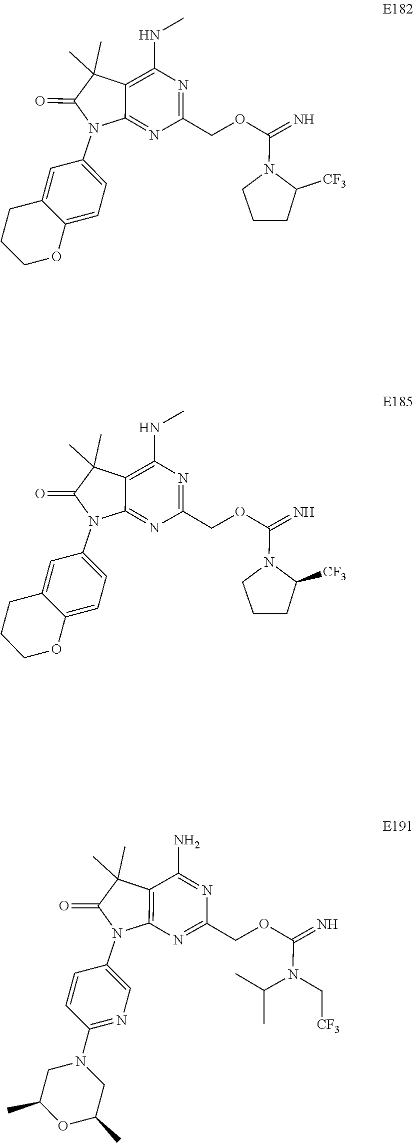 [SMILES: CNc1nc(COC(=N)N2CCCC2C(F)(F)F)nc2c1C(C)(C)C(=O)N2c1ccc2c(c1)CCCO2 ; CNc1nc(COC(=N)N2CCC[C@@H]2C(F)(F)F)nc2c1C(C)(C)C(=O)N2c1ccc2c(c1)CCCO2 ; CC(C)N(CC(F)(F)F)C(=N)OCc1nc(N)c2c(n1)N(c1ccc(N3C[C@@H](C)O[C@@H](C)C3)nc1)C(=O)C2(C)C]

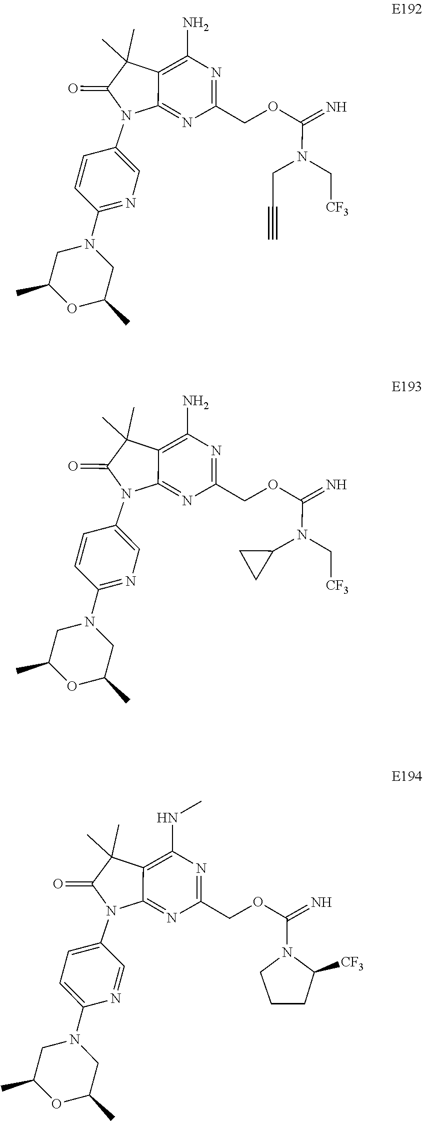 [SMILES: C#CCN(CC(F)(F)F)C(=N)OCc1nc(N)c2c(n1)N(c1ccc(N3C[C@@H](C)O[C@@H](C)C3)nc1)C(=O)C2(C)C ; C[C@@H]1CN(c2ccc(N3C(=O)C(C)(C)c4c(N)nc(COC(=N)N(CC(F)(F)F)C5CC5)nc43)cn2)C[C@H](C)O1 ; CNc1nc(COC(=N)N2CCC[C@@H]2C(F)(F)F)nc2c1C(C)(C)C(=O)N2c1ccc(N2C[C@@H](C)O[C@@H](C)C2)nc1]

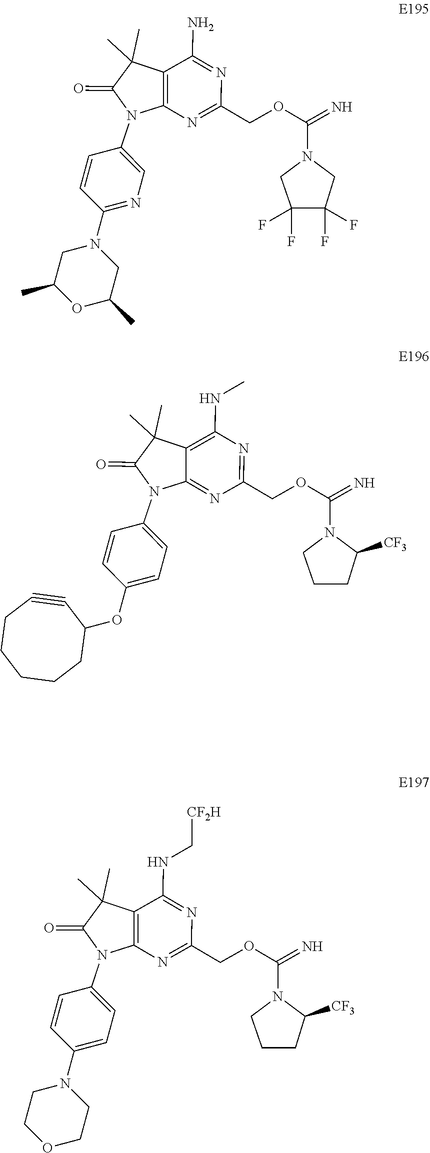 [SMILES: C[C@@H]1CN(c2ccc(N3C(=O)C(C)(C)c4c(N)nc(COC(=N)N5CC(F)(F)C(F)(F)C5)nc43)cn2)C[C@H](C)O1 ; CNc1nc(COC(=N)N2CCC[C@@H]2C(F)(F)F)nc2c1C(C)(C)C(=O)N2c1ccc(OC2C#CCCCCC2)cc1 ; CC1(C)C(=O)N(c2ccc(N3CCOCC3)cc2)c2nc(COC(=N)N3CCC[C@@H]3C(F)(F)F)nc(NCC(F)F)c21]

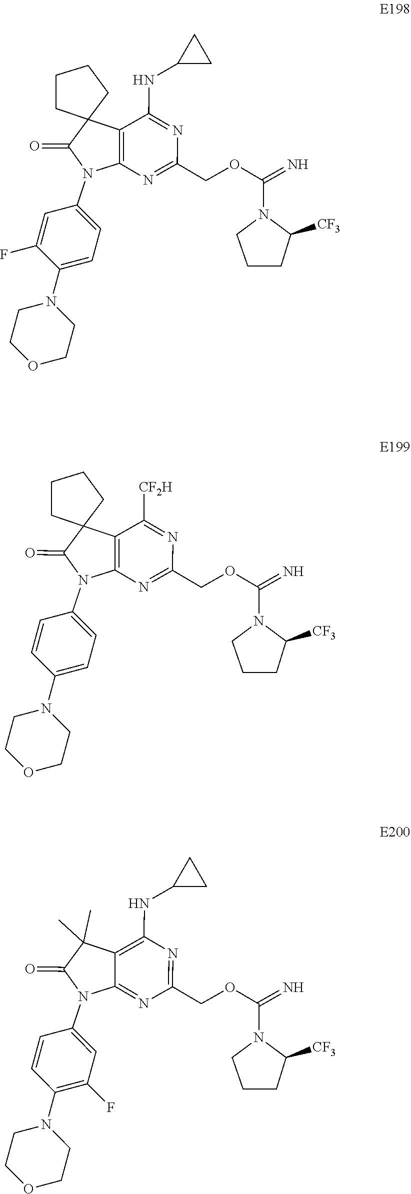 [SMILES: N=C(OCc1nc(NC2CC2)c2c(n1)N(c1ccc(N3CCOCC3)c(F)c1)C(=O)C21CCCC1)N1CCC[C@@H]1C(F)(F)F ; N=C(OCc1nc(C(F)F)c2c(n1)N(c1ccc(N3CCOCC3)cc1)C(=O)C21CCCC1)N1CCC[C@@H]1C(F)(F)F ; CC1(C)C(=O)N(c2ccc(N3CCOCC3)c(F)c2)c2nc(COC(=N)N3CCC[C@@H]3C(F)(F)F)nc(NC3CC3)c21]

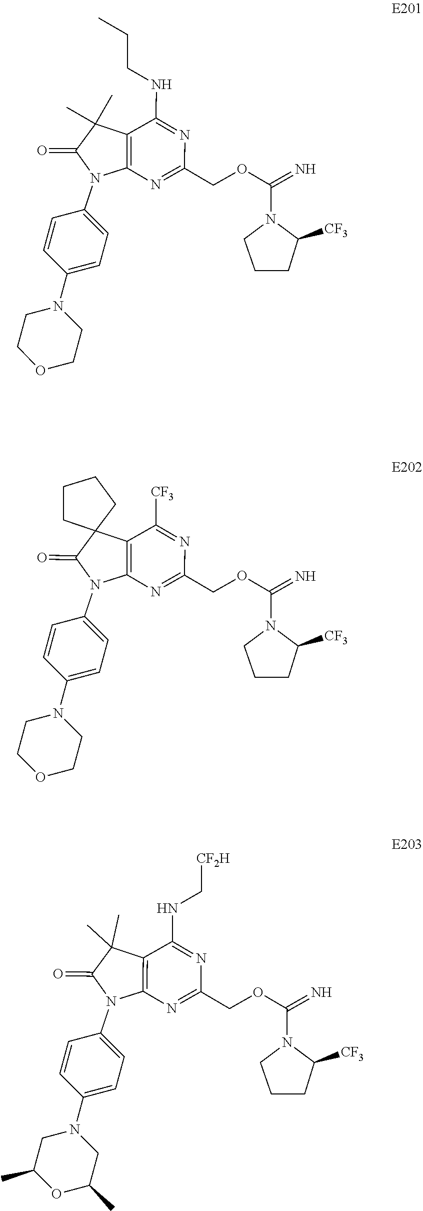 [SMILES: CCCNc1nc(COC(=N)N2CCC[C@@H]2C(F)(F)F)nc2c1C(C)(C)C(=O)N2c1ccc(N2CCOCC2)cc1 ; N=C(OCc1nc2c(c(C(F)(F)F)n1)C1(CCCC1)C(=O)N2c1ccc(N2CCOCC2)cc1)N1CCC[C@@H]1C(F)(F)F ; C[C@@H]1CN(c2ccc(N3C(=O)C(C)(C)c4c(NCC(F)F)nc(COC(=N)N5CCC[C@@H]5C(F)(F)F)nc43)cc2)C[C@H](C)O1]

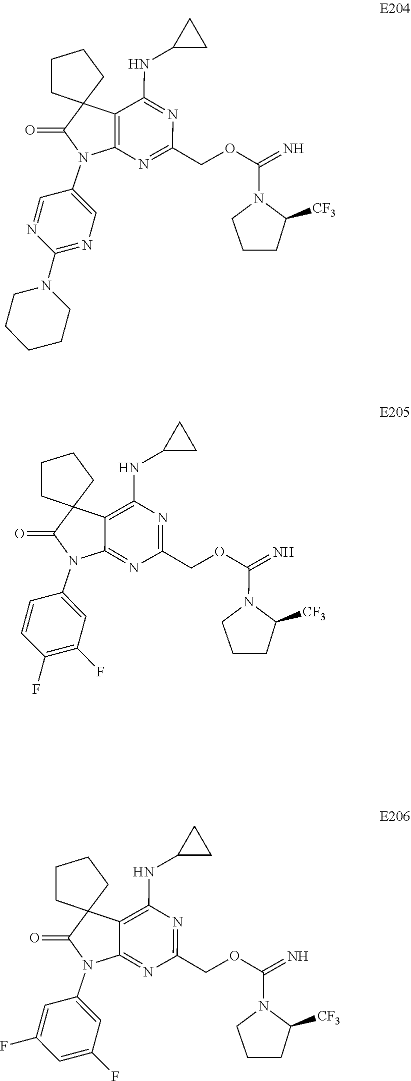 [SMILES: N=C(OCc1nc(NC2CC2)c2c(n1)N(c1cnc(N3CCCCC3)nc1)C(=O)C21CCCC1)N1CCC[C@@H]1C(F)(F)F ; N=C(OCc1nc(NC2CC2)c2c(n1)N(c1ccc(F)c(F)c1)C(=O)C21CCCC1)N1CCC[C@@H]1C(F)(F)F ; N=C(OCc1nc(NC2CC2)c2c(n1)N(c1cc(F)cc(F)c1)C(=O)C21CCCC1)N1CCC[C@@H]1C(F)(F)F]

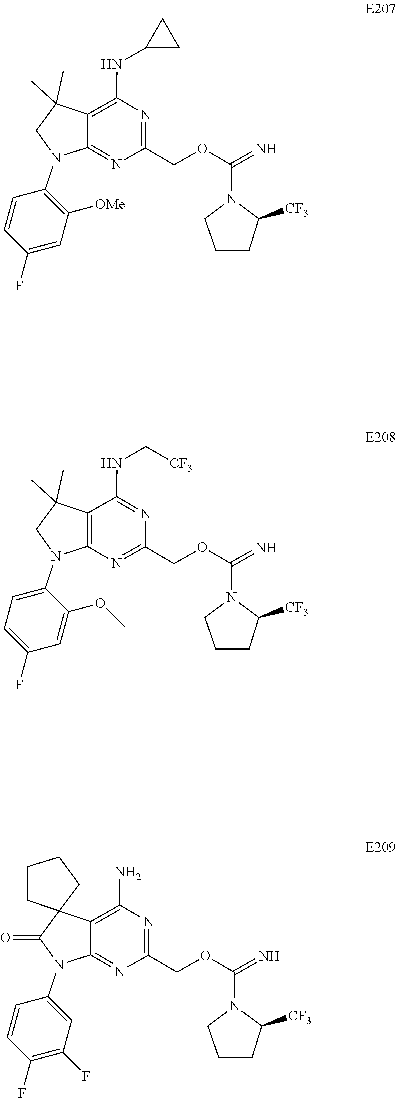 [SMILES: COc1cc(F)ccc1N1CC(C)(C)c2c(NC3CC3)nc(COC(=N)N3CCC[C@@H]3C(F)(F)F)nc21 ; COc1cc(F)ccc1N1CC(C)(C)c2c(NCC(F)(F)F)nc(COC(=N)N3CCC[C@@H]3C(F)(F)F)nc21 ; N=C(OCc1nc(N)c2c(n1)N(c1ccc(F)c(F)c1)C(=O)C21CCCC1)N1CCC[C@@H]1C(F)(F)F]

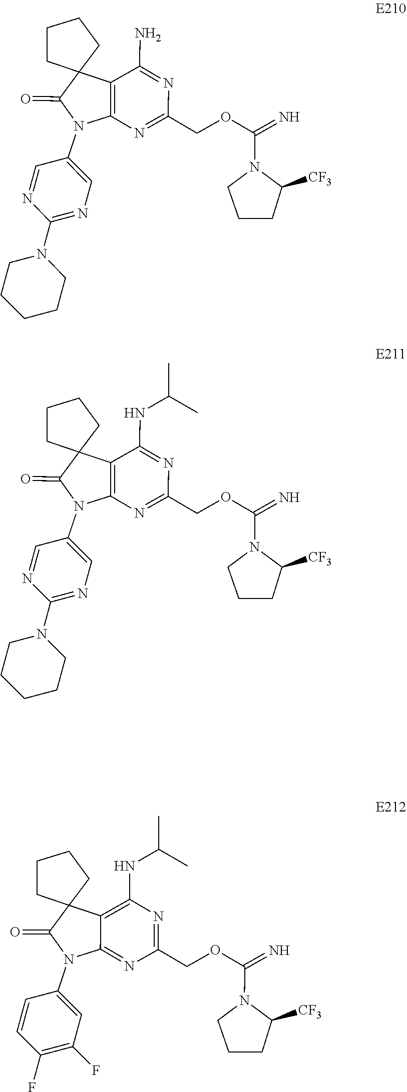 [SMILES: N=C(OCc1nc(N)c2c(n1)N(c1cnc(N3CCCCC3)nc1)C(=O)C21CCCC1)N1CCC[C@@H]1C(F)(F)F ; CC(C)Nc1nc(COC(=N)N2CCC[C@@H]2C(F)(F)F)nc2c1C1(CCCC1)C(=O)N2c1cnc(N2CCCCC2)nc1 ; CC(C)Nc1nc(COC(=N)N2CCC[C@@H]2C(F)(F)F)nc2c1C1(CCCC1)C(=O)N2c1ccc(F)c(F)c1]

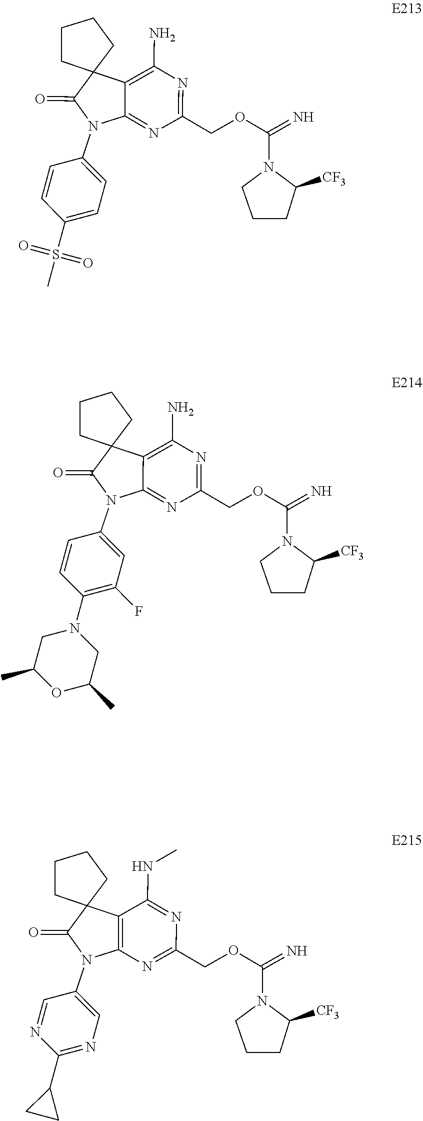 [SMILES: CS(=O)(=O)c1ccc(N2C(=O)C3(CCCC3)c3c(N)nc(COC(=N)N4CCC[C@@H]4C(F)(F)F)nc32)cc1 ; C[C@@H]1CN(c2ccc(N3C(=O)C4(CCCC4)c4c(N)nc(COC(=N)N5CCC[C@@H]5C(F)(F)F)nc43)cc2F)C[C@H](C)O1 ; CNc1nc(COC(=N)N2CCC[C@@H]2C(F)(F)F)nc2c1C1(CCCC1)C(=O)N2c1cnc(C2CC2)nc1]

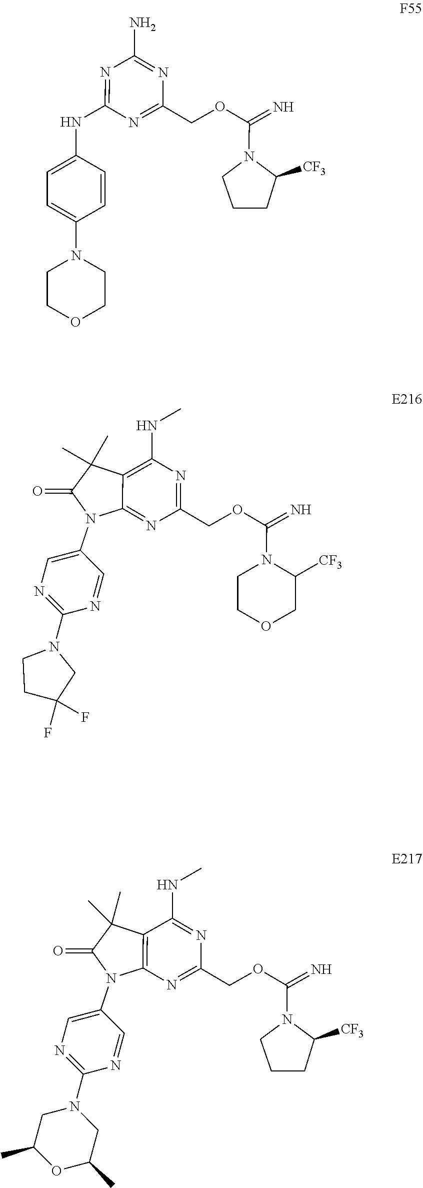 [SMILES: N=C(OCc1nc(N)nc(Nc2ccc(N3CCOCC3)cc2)n1)N1CCC[C@@H]1C(F)(F)F ; CNc1nc(COC(=N)N2CCOCC2C(F)(F)F)nc2c1C(C)(C)C(=O)N2c1cnc(N2CCC(F)(F)C2)nc1 ; CNc1nc(COC(=N)N2CCC[C@@H]2C(F)(F)F)nc2c1C(C)(C)C(=O)N2c1cnc(N2C[C@@H](C)O[C@@H](C)C2)nc1]

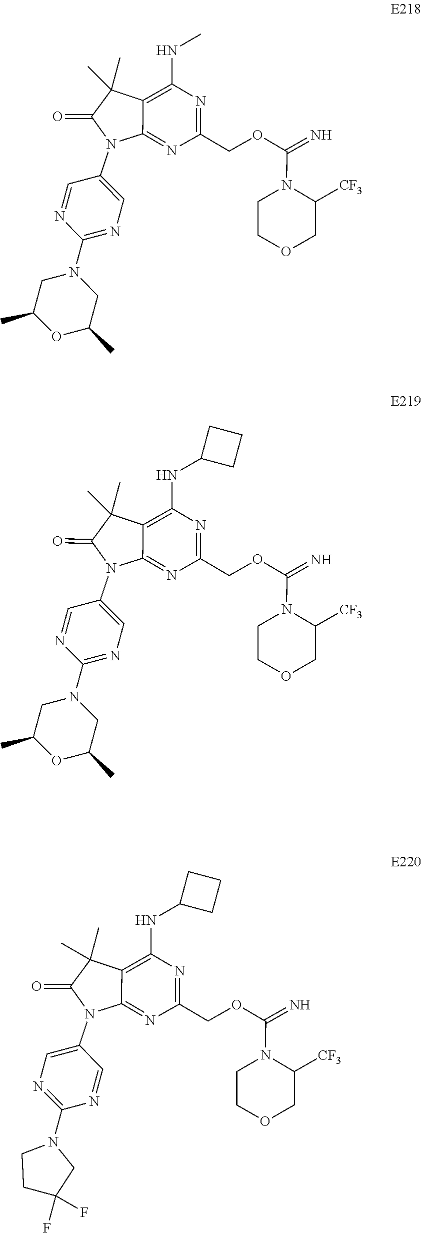 [SMILES: CNc1nc(COC(=N)N2CCOCC2C(F)(F)F)nc2c1C(C)(C)C(=O)N2c1cnc(N2C[C@@H](C)O[C@@H](C)C2)nc1 ; C[C@@H]1CN(c2ncc(N3C(=O)C(C)(C)c4c(NC5CCC5)nc(COC(=N)N5CCOCC5C(F)(F)F)nc43)cn2)C[C@H](C)O1 ; CC1(C)C(=O)N(c2cnc(N3CCC(F)(F)C3)nc2)c2nc(COC(=N)N3CCOCC3C(F)(F)F)nc(NC3CCC3)c21]

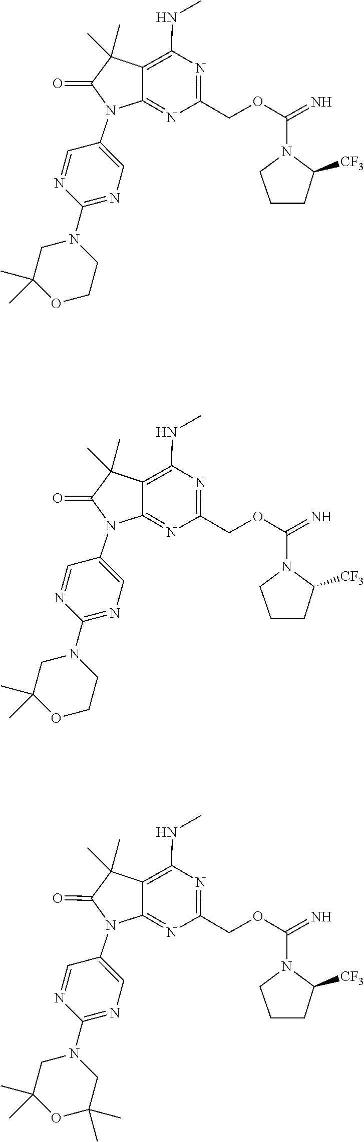 [SMILES: CNc1nc(COC(=N)N2CCC[C@@H]2C(F)(F)F)nc2c1C(C)(C)C(=O)N2c1cnc(N2CCOC(C)(C)C2)nc1 ; CNc1nc(COC(=N)N2CCC[C@H]2C(F)(F)F)nc2c1C(C)(C)C(=O)N2c1cnc(N2CCOC(C)(C)C2)nc1 ; CNc1nc(COC(=N)N2CCC[C@@H]2C(F)(F)F)nc2c1C(C)(C)C(=O)N2c1cnc(N2CC(C)(C)OC(C)(C)C2)nc1]

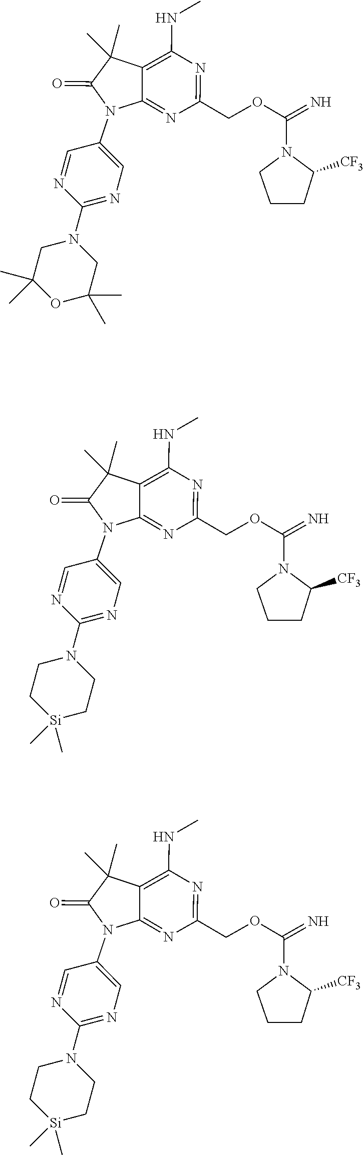 [SMILES: CNc1nc(COC(=N)N2CCC[C@H]2C(F)(F)F)nc2c1C(C)(C)C(=O)N2c1cnc(N2CC(C)(C)OC(C)(C)C2)nc1 ; CNc1nc(COC(=N)N2CCC[C@@H]2C(F)(F)F)nc2c1C(C)(C)C(=O)N2c1cnc(N2CC[Si](C)(C)CC2)nc1 ; CNc1nc(COC(=N)N2CCC[C@H]2C(F)(F)F)nc2c1C(C)(C)C(=O)N2c1cnc(N2CC[Si](C)(C)CC2)nc1]

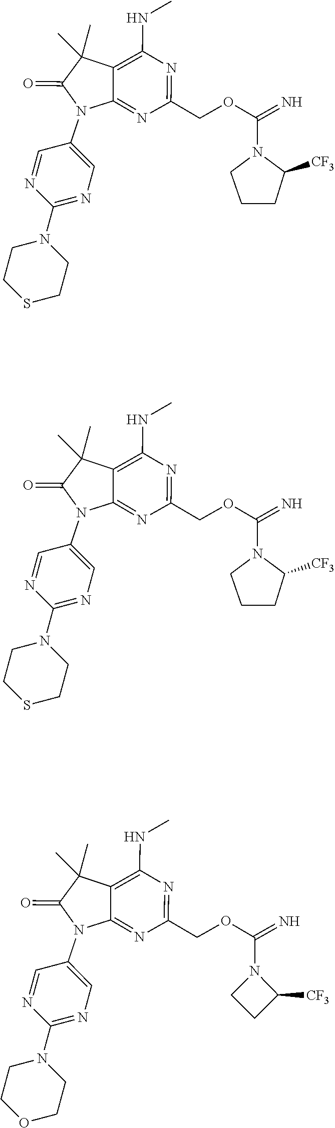 [SMILES: CNc1nc(COC(=N)N2CCC[C@@H]2C(F)(F)F)nc2c1C(C)(C)C(=O)N2c1cnc(N2CCSCC2)nc1 ; CNc1nc(COC(=N)N2CCC[C@H]2C(F)(F)F)nc2c1C(C)(C)C(=O)N2c1cnc(N2CCSCC2)nc1 ; CNc1nc(COC(=N)N2CC[C@@H]2C(F)(F)F)nc2c1C(C)(C)C(=O)N2c1cnc(N2CCOCC2)nc1]

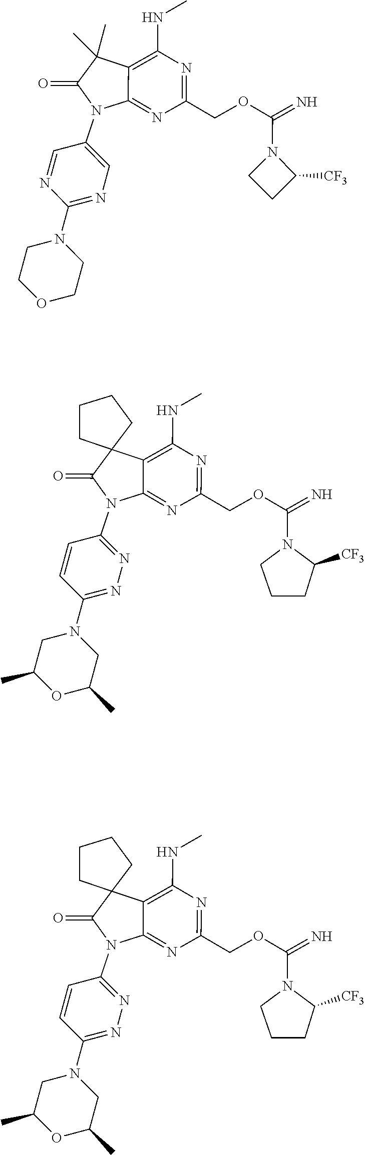 [SMILES: CNc1nc(COC(=N)N2CC[C@H]2C(F)(F)F)nc2c1C(C)(C)C(=O)N2c1cnc(N2CCOCC2)nc1 ; CNc1nc(COC(=N)N2CCC[C@@H]2C(F)(F)F)nc2c1C1(CCCC1)C(=O)N2c1ccc(N2C[C@@H](C)O[C@@H](C)C2)nn1 ; CNc1nc(COC(=N)N2CCC[C@H]2C(F)(F)F)nc2c1C1(CCCC1)C(=O)N2c1ccc(N2C[C@@H](C)O[C@@H](C)C2)nn1]

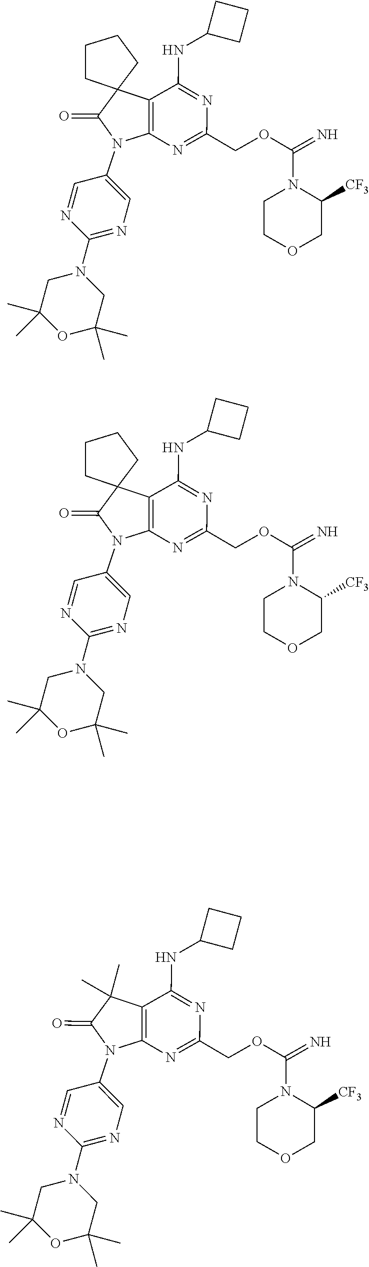 [SMILES: CC1(C)CN(c2ncc(N3C(=O)C4(CCCC4)c4c(NC5CCC5)nc(COC(=N)N5CCOC[C@@H]5C(F)(F)F)nc43)cn2)CC(C)(C)O1 ; CC1(C)CN(c2ncc(N3C(=O)C4(CCCC4)c4c(NC5CCC5)nc(COC(=N)N5CCOC[C@H]5C(F)(F)F)nc43)cn2)CC(C)(C)O1 ; CC1(C)CN(c2ncc(N3C(=O)C(C)(C)c4c(NC5CCC5)nc(COC(=N)N5CCOC[C@@H]5C(F)(F)F)nc43)cn2)CC(C)(C)O1]

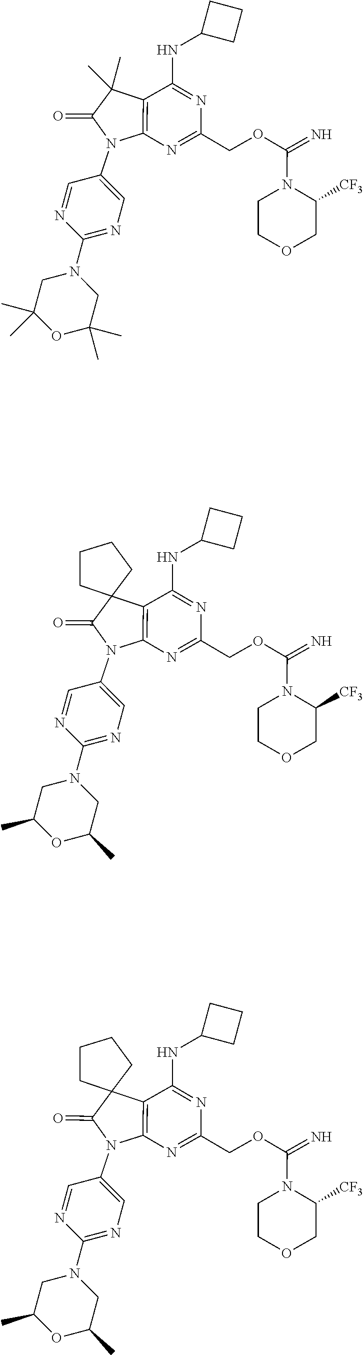 [SMILES: CC1(C)CN(c2ncc(N3C(=O)C(C)(C)c4c(NC5CCC5)nc(COC(=N)N5CCOC[C@H]5C(F)(F)F)nc43)cn2)CC(C)(C)O1 ; C[C@@H]1CN(c2ncc(N3C(=O)C4(CCCC4)c4c(NC5CCC5)nc(COC(=N)N5CCOC[C@@H]5C(F)(F)F)nc43)cn2)C[C@H](C)O1 ; C[C@@H]1CN(c2ncc(N3C(=O)C4(CCCC4)c4c(NC5CCC5)nc(COC(=N)N5CCOC[C@H]5C(F)(F)F)nc43)cn2)C[C@H](C)O1]

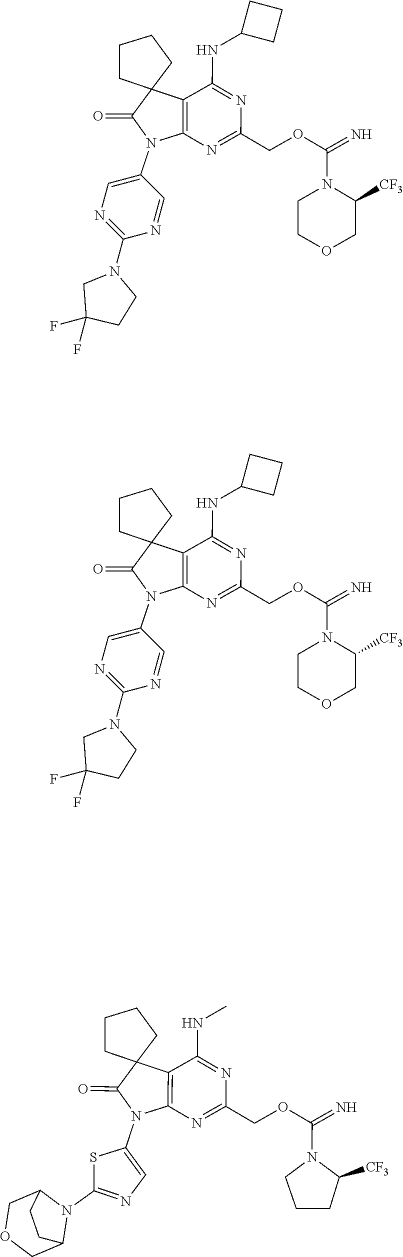 [SMILES: N=C(OCc1nc(NC2CCC2)c2c(n1)N(c1cnc(N3CCC(F)(F)C3)nc1)C(=O)C21CCCC1)N1CCOC[C@@H]1C(F)(F)F ; N=C(OCc1nc(NC2CCC2)c2c(n1)N(c1cnc(N3CCC(F)(F)C3)nc1)C(=O)C21CCCC1)N1CCOC[C@H]1C(F)(F)F ; CNc1nc(COC(=N)N2CCC[C@@H]2C(F)(F)F)nc2c1C1(CCCC1)C(=O)N2c1cnc(N2C3CCC2COC3)s1]

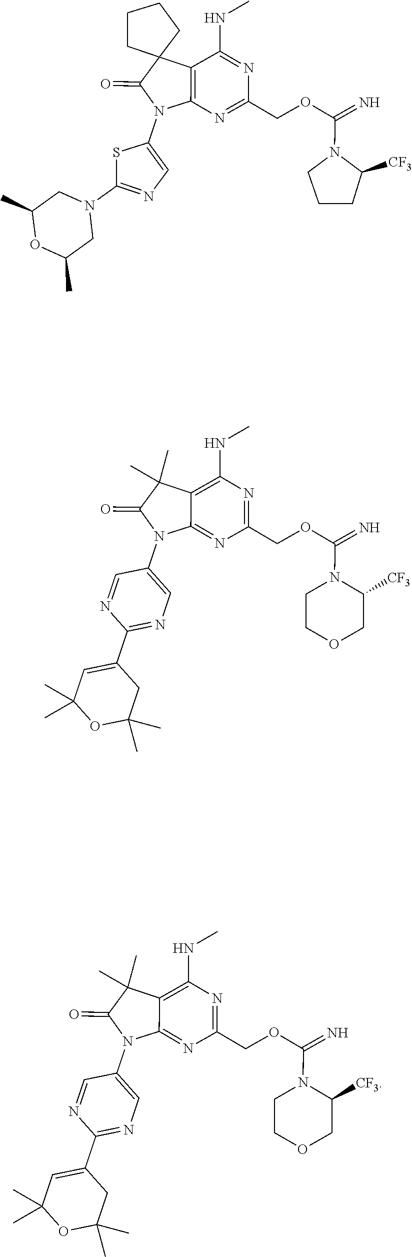 [SMILES: CNc1nc(COC(=N)N2CCC[C@@H]2C(F)(F)F)nc2c1C1(CCCC1)C(=O)N2c1cnc(N2C[C@@H](C)O[C@@H](C)C2)s1 ; CNc1nc(COC(=N)N2CCOC[C@H]2C(F)(F)F)nc2c1C(C)(C)C(=O)N2c1cnc(C2=CC(C)(C)OC(C)(C)C2)nc1 ; CNc1nc(COC(=N)N2CCOC[C@@H]2C(F)(F)F)nc2c1C(C)(C)C(=O)N2c1cnc(C2=CC(C)(C)OC(C)(C)C2)nc1]

### Production

Among other things, the present disclosure provides technologies, e.g., reagents, conditions, intermediates, etc., for manufacturing provided compounds.

In some embodiments, the present disclosure provides a method, comprising reacting a first compound comprising a OH group or a salt thereof with a second compound comprising CN to form an isourea moiety. In some embodiments, a first compound is a compound, e.g., of formula A, B, C, D, E, F, G, H, etc. wherein R⁴ is R⁴ is -L^{w}-R^{wh} wherein each variable is independently as described herein. In some embodiments, R⁴ is -L^{w}-OH or a salt thereof wherein L^{w} is as described herein. In some embodiments, R⁴ is -L^{w}-SH or a salt thereof wherein L^{w} is as described herein. In some embodiments, a second compound has the structure of N(CN)(R^{w2})(R^{w3}) or a salt thereof, wherein each of R^{w2} and R^{w3} is independently as described herein. In some embodiments, the present disclosure provides a compound having the structure of N(CN)(R^{w2})(R^{w3}) or a salt thereof, wherein:
each of R^{w2} and R^{w3} is independently -L-R′ or a detectable label, wherein:
T is O or S;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R′ is independently R, —OR, —C(O)R, —C(O)OR, —C(O)N(R)₂, —S(O)R, or —S(O)OR; and
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, the present disclosure provides a method, comprising reacting a first compound comprising a —OH group or a salt thereof with a second compound comprising —N═C═N— to form an isourea moiety. In some embodiments, a first compound is a compound, e.g., of formula A, B, C, D, E, F, G, H, etc. wherein R⁴ is R⁴ is -L^{w}-R^{wh} wherein each variable is independently as described herein. In some embodiments, R⁴ is -L^{w}-OH or a salt thereof wherein L^{w} is as described herein. In some embodiments, R⁴ is -L^{w}-SH or a salt thereof wherein L^{w} is as described herein. In some embodiments, a second compound has the structure of R^{w1}—N═C═N—R^{w2} or R^{w1}—N═C═N—R^{w3} or a salt thereof, wherein each variable is independently as described herein. In some embodiments, the present disclosure provides a compound having the structure of R^{w1}—N═C═N—R^{w3}, wherein:
each of R^{w1} and R^{w3} is independently -L-R′ or a detectable label, wherein:
T is O or S;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R′ is independently R, —OR, —C(O)R, —C(O)OR, —C(O)N(R)₂, —S(O)R, or —S(O)OR; and
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

In some embodiments, the present disclosure provides a method, comprising reacting a first compound comprising a nucleophilic group or a salt thereof with a thiourea compound. In some embodiments, a thiourea compound has the structure of T=C(NHR^{w1})N(R^{w2})(R^{w3}), wherein T is S and each of R^{w1}, R^{w2} and R^{w3} is independently as described herein. In some embodiments, a first compound is a compound having the structure of formula A, B, C, D, E, F, G, H, etc. or a salt thereof, wherein R⁴ is -L^{w}-R^{w}′, wherein R^{w}′ is a leaving group, e.g., —Cl, —Br, —I, —OS(O)₂R wherein R is not —H and is as described herein (e.g., optionally substituted phenyl). In some embodiments, R^{w}′ is —Cl. In some embodiments, R^{w}′ is —Br. In some embodiments, R^{w}′ is —I.

In some embodiments, a method is performed in the presence of a Lewis acid. Various Lewis acid are known in the art and may be assessed and utilized in accordance with the present disclosure. In some embodiments, a Lewis acid comprises a metal ion. In some embodiments, a Lewis acid comprises a Zn, Sn or Cu ion. In some embodiments, a Lewis acid is ZnCl₂, SnCl₄, CuCl₂, Zn(OAc)₂ or Cu(OTf)₂.

Additionally or alternatively, in some embodiments, reacting is performed in the presence of a base. In some embodiments, a base is a sodium or lithium base. In some embodiments, a base is NaH, Cs₂CO₃, LDA, or LiHMDS. Many other bases are known in the art and may be assessed and utilized in accordance with the present disclosure.

In some embodiments, R^{w1} is —H. In some embodiments, the present disclosure provides a method, comprising reacting a compound, e.g., of formula A, B, C, D, E, F, G, H, etc., wherein R^{w1} is —H with an electrophile. In some embodiments, an electrophile is R^{c}-LG or a salt thereof, wherein R^{c} is R as described herein but is not —H, and LG is a leaving group, e.g., —Cl, —Br, —I, —OS(O)₂R wherein R is not —H and is as described herein, etc. In some embodiments, LG is —Cl. In some embodiments, LG is —Br. In some embodiments, LG is —I. In some embodiments, LG is —OS(O)₂R wherein R is not —H and is as described herein (e.g., optionally substituted phenyl). In some embodiments, R^{∘} is optionally substituted C₁₋₆ aliphatic. In some embodiments, an electrophile is CH₃1.

As appreciated by those skilled in the art, in chemical reactions various groups, e.g., hydroxyl, amino, carboxyl, etc. may be protected to avoid undesired reactions. Many technologies for protection/de-protection are available to those skilled in the art and may be utilized in accordance with the present disclosure. Certain such technologies are described herein including exemplified in the Examples.

Various chemical reactions are typically performed in a solvent. In some embodiments, a reaction is performed in a single solvent, e.g., DCM, THF, Et₂O, EtOH, toluene, etc. In some embodiments, a reaction is performed in a mixture of two or more solvents. In some embodiments, a solvent is polar. In some embodiments, a solvent is non-polar. In some embodiments, a solvent is protic. In some embodiments, a solvent is non-protic. In some embodiments, a solvent is polar but is not protic. In some embodiments, a solvent is or comprises water. Suitable solvents for various reactions are available to those skilled in the art and can be utilized in accordance with the present disclosure.

In some embodiments, a reaction is conducted under an inert atmosphere, e.g., N₂, Ar, etc. In some embodiments, a reaction is conducted with exposure to air. In some embodiments, a reaction is conducted under anhydrous conditions, e.g., with reagents, solvents, vessels, etc., properly dried. In some embodiments, a reaction is conducted in the presence of significant of water (e.g., about or more than about 0.1, 0.5, or 1 equivalent).

Reactions may be performed at various temperatures. In some embodiments, a reaction is performed at a temperature lower than about room temperature, e.g., about or no more than about −78, −60, −50, −40, −30, −20, −10, 0 or 10° C. In some embodiments, a temperature is about or no more than about 10° C. In some embodiments, a temperature is about or no more than about 15° C. In some embodiments, a temperature is about or no more than about 20° C. In some embodiments, a reaction temperature is about room temperature. In some embodiments, a reaction temperature is higher than room temperature. In some embodiments, a reaction temperature is about or at least about 35, 40, 50, 60, 70, 80, 90, 100, 100, 110, 120, 150° C., etc. In some embodiments, a reaction comprises refluxing in a solvent, e.g., in ether, toluene, etc. In some embodiments, temperature changes during a reaction process, e.g., increasing from a lower temperature to a higher temperature, decreasing from a higher temperature to a lower temperature, or both.

In some embodiments, the present disclosure provides compounds of high purity. In some embodiments, purity of a compound is or greater than about 85%, 90%, 91%, 92%, 93%, 94%, 95%, 96%, 97%, 98%, 99%, 99.7%, or 99.9%. In some embodiments, purity of a compound is or greater than about 85%. In some embodiments, purity of a compound is or greater than about 85%. In some embodiments, purity of a compound is or greater than about 90%. In some embodiments, purity of a compound is or greater than about 95%. In some embodiments, purity of a compound is or greater than about 96%. In some embodiments, purity of a compound is or greater than about 97%. In some embodiments, purity of a compound is or greater than about 98%. In some embodiments, purity of a compound is or greater than about 99%. In some embodiments, purity of a compound is or greater than about 99.7%. In some embodiments, purity of a compound is or greater than about 99.9%.

In some embodiments, a product is selectively produced over another potential product. In some embodiments, a product is produced with chemoselectivity, stereoselectivity and/or regioselectivity. In some embodiments, a selectivity is presented as a ratio, e.g., of one product over another. In some embodiments, a ratio is about or at least about 1.5:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 40:1, 50:1, 60:1, 70:1, 80:1, 90:1, 100:1, 200:1, 500:1 or more.

Reactions may be performed for a variety of time lengths. In some embodiments, reactions complete instantly. In some embodiments, reaction times varies from minutes to hours to days, e.g., 5, 10, 15, 20, 30, 45 minutes, or 1, 1.5, 2, 3, 4, 5, 6, 7, 8, 9, 10, 12, 15, 18, 20 or 22 hours, or one or two days or longer. Those skilled in the art can use various technologies to determine when to terminate reactions, e.g., based on consumption of starting materials, products formation, by-products formation, etc.

### Characterization and Assessment

Those skilled in the art reading the present disclosure will appreciate that various technologies are available and may be utilized to assess provided technologies, e.g., compounds, compositions, methods, etc. Certain useful technologies are described in the Examples herein. In some embodiments, provided technologies are assessed in intro. In some embodiments, provided technologies are assessed in vivo. In some embodiments, provided technologies are assessed in animal models for conditions, disorders or diseases. In some embodiments, provided technologies are assessed in clinical trials involving human subjects. Certain useful technologies are described in, e.g., WO 2014/011973, WO 2018/218087, WO 2019/168999, WO 2020/176757, WO 2021/041536, WO 2021/041539, WO 2021/183702, WO 2022/042657, Eaton J K, Furst L, Cai L L, Viswanathan V S, Schreiber S L., Structure-activity relationships of GPX4 inhibitor warheads, Bioorg Med Chem Lett. 2020 Dec. 1; 30(23):127538. doi: 10.1016/j.bmcl.2020.127538, Shimada K, Skouta R, Kaplan A, Yang W S, Hayano M, Dixon S J, Brown L M, Valenzuela C A, Wolpaw A J, Stockwell B R, Global survey of cell death mechanisms reveals metabolic regulation of ferroptosis, Nature Chemical Biology, 2016 May 9; 12: 497-503. doi: 10.1038/nchembio.2079, etc. In some embodiments, a technology comprises utilization of cell lines. In some embodiments, a technology comprises administration or delivery of a compound to a subject, e.g., a human, an animal (e.g., mouse, monkey, etc.). In some embodiments, a technology comprises an animal model.

In some embodiments, provided compounds or compositions may be assessed in an assay for their capability to inhibit cell growth or proliferation, e.g., through inducing or promoting ferroptosis. In some embodiments, assessment is performed further in the presence of a ferroptosis inhibitor, e.g., those described in the Examples or available in the art. In some embodiments, an effect of the compound, e.g., inducing ferroptosis, inhibiting cell proliferation, etc., is reduced in the presence of a ferroptosis inhibitor. In some embodiments, inhibitory activities of provided compounds are presented as IC50 values. In some embodiments, IC50 of a compound, e.g., when assessed in an assay described in an Example, is about or no more than about 100, 90, 80, 70, 60, 50, 40, 30, 20, 10, 9, 8, 7, 6, 5, 4, 3, 2, or 1 uM, and in some embodiments, is about or no more than about 900, 800, 700, 600, 500, 400, 300, 200, 100, 90, 80, 70, 60, 50, 40, 30, 20, 10, 9, 8, 7, 6, 5, 4, 3, 2, or 1 nM. In some embodiments, when assessed in the presence of a ferroptosis inhibitor, e.g., as described in an Example, IC50 of a compound is about or at least about 2-5000, e.g., about or at least about 10-1000, 50-1000, 100-1000, 2, 3, 4, 5, 6, 7, 8, 9, 10, 15, 20, 25, 30, 40, 50, 60, 70, 80, 90, 100, 200, 500 or 1000, fold of the IC50 under comparable or identical conditions but absent of the ferroptosis inhibitor. In some embodiments, it is about or at least about 5 fold. In some embodiments, it is about or at least about 10 fold. In some embodiments, it is about or at least about 20 fold. In some embodiments, it is about or at least about 50 fold. In some embodiments, it is about or at least about 100 fold. In some embodiments, IC50 of a compound in the presence of a ferroptosis inhibitor, e.g., when assessed in an assay described in an Example, is about or at least about 1-1000, e.g., about or at least about 1-500, 1-100, 10-1000, 100-1000, 1, 2, 3, 4, 5, 6, 7, 8, 9, 10, 15, 20, 25, 30, 40, 50, 60, 70, 80, 90, 100, 200, 500, or 1000 uM. In some embodiments, concentration of a ferroptosis inhibitor is about or at least about EC50 of a ferroptosis inhibitor when it is assessed by itself. In some embodiments, concentration of a ferroptosis inhibitor is about or at least about 0.1-100, e.g., about or at least about 1-20, 2-10, 0.1, 0.2, 0.3, 0.4, 0.5, 0.6, 0.7, 0.8, 0.9, 1, 1.5, 2, 3, 4, 5, 6, 7, 8, 9, 10, 20, 30, 40, 50, 60, 70, 80, 90 or 100 uM. In some embodiments, concentration of the ferroptosis inhibitor is about or at least about 1 uM. In some embodiments, concentration of the ferroptosis inhibitor is about or at least about 2 uM. Certain useful assays and data are presented in the Examples.

Various compounds are of sufficient stability for therapeutic uses. Various technologies are available in the art and can be utilized in accordance with the present disclosure for assessing stability; certain examples are described in the Examples. In some embodiments, stability may be presented as % remaining compound in whole blood after a period of incubation under a condition. In some embodiments, % remaining of a compound in human whole blood after 120 mins incubation is about or at least about 5%, 10%, 15%, 20%, 25%, 30%, 35%, 40%, 45%, 50%, 55%, 60%, 65%, 70%, 75%, 80%, 85%, 90%, 95%, or 100%. In some embodiments, it is about or at least about 10%. In some embodiments, it is about or at least about 20%. In some embodiments, it is about or at least about 25%. In some embodiments, it is about or at least about 30%. In some embodiments, it is about or at least about 40%. In some embodiments, it is about or at least about 50%. In some embodiments, it is about or no more than about 30%, 35%, 40%, 45%, 50%, 55%, 60%, 65%, 70%, 75%, 80%, 85%, 90%, 95%, or 100%. In some embodiments, it is about or no more than about 30%. In some embodiments, it is about or no more than about 40%. In some embodiments, it is about or no more than about 50%. In some embodiments, it is about or no more than about 60%. In some embodiments, it is about or no more than about 70%. In some embodiments, it is about or no more than about 80%. In some embodiments, it is about or no more than about 90%. In some embodiments, it is about or no more than about 95%.

In some embodiments, provided technologies provides selectivity, e.g., for reacting with one compound over another, for reacting one nucleophilic group (e.g., —SeH or a salt form thereof) over another (e.g., —SH or a salt form thereof), for reacting with one amino acid residue (e.g. selenocysteine) over another (cysteine), for delivering a compound to a target site over a reference site, for producing one isomer over another, for isotope-labeling one position in a compound over another, etc. In some embodiments, a selectivity is presented as a ratio, e.g., desired vs. non-desired. In some embodiments, a ratio is about or at least about 1.5:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 40:1, 50:1, 60:1, 70:1, 80:1, 90:1, 100:1, 200:1, 500:1 or more. In some embodiments, it is about or at least about 2. In some embodiments, it is about or at least about 5. In some embodiments, it is about or at least about 10. In some embodiments, it is about or at least about 20. In some embodiments, it is about or at least about 50. In some embodiments, it is about or at least about 100. Various technologies are available in the art and can be utilized in accordance with the present disclosure. For example, in some embodiments, to assess selectivity for selenocysteine over cysteine residues, mass spectrometry may be utilized.

In some embodiments, the present disclosure provides technologies for identifying compounds useful for various applications. In some embodiments, the present disclosure provides a method, comprising:
assessing interactions of a plurality of compounds with a polypeptide comprising —SeH or a salt form thereof, wherein one or more compounds are independently a compound described herein, and
identifying one or more compounds that can bind to the polypeptide.

In some embodiments, a polypeptide comprises a selenocysteine residue. In some embodiments, a polypeptide comprises a characteristic sequence element of GPX4. In some embodiments, a polypeptide comprises a characteristic sequence element of GPX4 and U73. In some embodiments, a polypeptide is GPX4. In some embodiments, assessing comprises assessment performed in silico. In some embodiments, assessing comprises assessment performed in vitro. In some embodiments, assessing comprises assessment performed in vivo. In some embodiments, a compound can bind to a polypeptide through covalent bonding to an amino acid residue, e.g., a selenocysteine residues.

### Applications

Provided technologies are useful for various applications. In some embodiments, provided technologies are useful for modulating a biological activity. In some embodiments, provided technologies are useful for modulating properties and/or activities of polypeptides. In some embodiments, provided technologies are useful for inhibiting activities of polypeptides. In some embodiments, provided technologies are useful for preventing or treating various conditions, disorders or diseases.

In some embodiments, the present disclosure provides moieties, e.g., isourea moieties, isothiourea moieties, R^{w}, etc., that can be utilized as leaving groups. In some embodiments, the present disclosure provides moieties that can be utilized as “warheads.” As those skilled in the art reading the present disclosure will appreciate, warhead moieties can be utilized with many other moieties to provide compounds for various purposes, for example, protein inhibitors including covalent inhibitors. In some embodiments, an inhibitor is a kinase inhibitor. In some embodiments, an inhibitor is a GPX4 inhibitor. In some embodiments, an inhibitor is a GPX4 inhibitor which forms a covalent bond with U73 of GPX4. In some embodiments, the present disclosure provides technologies for modifying, optimizing or improving a compound, comprising replacing an existing warhead moiety in the compound with a nucleophilic moiety described herein. In some embodiments, an existing warhead moiety is or comprises optionally substituted CH₂═CHC(O)—. In some embodiments, an existing warhead moiety is or comprises optionally substituted CHCC(O)—. In some embodiments, an existing warhead moiety is or comprises optionally substituted CH₂C₁C(O)—. In some embodiments, the present disclosure provides technologies for designing a compound, comprising combining a first moiety which can provide, promote or enhance binding to a target, e.g., a polypeptide, and an electrophilic moiety described herein. In some embodiments, the present disclosure provides compounds comprising a first moiety that can provide, promote or enhance binding to a target, and an electrophilic moiety as described herein. Various warhead moieties to be replaced and/or first moieties can be utilized are reported, including those described in WO 2014/011973, WO 2018/218087, WO 2019/168999, WO 2020/176757, WO 2021/041536, WO 2021/041539, WO 2021/183702, WO 2022/042657, Eaton J K, Furst L, Cai L L, Viswanathan V S, Schreiber S L., Structure-activity relationships of GPX4 inhibitor warheads, Bioorg Med Chem Lett. 2020 Dec. 1; 30(23):127538. doi: 10.1016/j.bmcl.2020.127538, Shimada K, Skouta R, Kaplan A, Yang W S, Hayano M, Dixon S J, Brown L M, Valenzuela C A, Wolpaw A J, Stockwell B R, Global survey of cell death mechanisms reveals metabolic regulation of ferroptosis, Nature Chemical Biology, 2016 May 9; 12: 497-503. doi: 10.1038/nchembio.2079, etc., and can be replaced and/or utilized in accordance with the present disclosure. In some embodiments, provided electrophilic moieties selectively react with certain types of nucleophiles over others. For example, in some embodiments, provided electrophilic moieties selectively react with —SeH or salt forms thereof over —SH or salt forms thereof. In some embodiments, provided electrophilic moieties selectively react with selenocysteine amino acid residues over cysteine amino acid residues. In some embodiments, a moiety of the present disclosure is an isourea moiety. In some embodiments, a moiety of the present disclosure is an isothiourea moiety. In some embodiments, a moiety of the present disclosure is R^{w}. In some embodiments, a provided moiety has the structure of -L^{w}-R^{w}, wherein each variable is independently as described herein. In some embodiments, R^{w} is -T-C(═NR^{w1})N(R^{w2})(R^{w3}). In some embodiments, R^{w} is —O—C(═NR^{w1})N(R^{w2})(R^{w3}). In some embodiments, L^{w} is optionally substituted —C(O)CH₂—, wherein the —CH₂— is bonded to R^{w}. In some embodiments, L^{w} is —C(O)CH₂—, wherein the —CH₂— is bonded to R^{w}. In some embodiments, L^{w} is optionally substituted —C(S)CH₂—, wherein the —CH₂— is bonded to R^{w}. In some embodiments, L^{w} is —C(S)CH₂—, wherein the —CH₂— is bonded to R^{w}. In some embodiments, L^{w} is optionally substituted —C(N(R′))CH₂—, wherein the —CH₂— is bonded to R^{w}. In some embodiments, L^{w} is —C(N(R′))CH₂—, wherein the —CH₂— is bonded to R^{w}.

In some embodiments, provided technologies are useful for labeling targets, e.g., polypeptides. In some embodiments, provided technologies are useful for delivering various agents to targets, e.g., cells, tissues, etc. In some embodiments, provided compounds have the structure of [PAYLOAD]-R⁴ or a structure thereof to deliver various payload agents. In some embodiments, [PAYLOAD] has such a structure that [PAYLOAD]-H is a payload. In some embodiments, R⁴ is -L^{w}-R^{w}, wherein each variable is independently as described herein. In some embodiments, a payload agent is a small molecule agent. In some embodiments, a payload agent is a drug. In some embodiments, a payload agent is or comprises a polypeptide agent. In some embodiments, a payload agent is or comprises an antibody agent. In some embodiments, a payload agent is or comprises a nucleic acid agent. In some embodiments, a payload agent is or comprises an oligonucleotide agent. In some embodiments, a payload agent is or comprises a carbohydrate agent. In some embodiments, a payload agent is or comprises a label. In some embodiments, a payload agent is or comprises a detectable moiety, e.g., a fluorescent moiety, a radioactive moiety, etc. In some embodiments, provided compounds react with nucleophiles, e.g., —SH, SeH, etc., or salt forms thereof. In some embodiments, provided compounds react with cysteine amino acid residues. In some embodiments, provided compounds react with selenocysteine residues, e.g., U73 of GPX4. In some embodiments, H—R^{w} is released (in some embodiments, as a tautomer thereof). In some embodiments, a released compound has the structure of T=C(NHR^{w1})N(R^{w2})(R^{w3}) or a salt thereof.

In some embodiments, the present disclosure provides technologies for delivering a compound to a target. In some embodiments, a delivered compound is a urea. In some embodiments, a delivered compound has the structure of H—R^{w} or a salt thereof (in some embodiments, in a tautomer form). In some embodiments, a delivered compound has the structure of T=C(NHR^{w1})N(R^{w2})(R^{w3}) or a salt thereof. In some embodiments, to deliver such a compound a compound comprising or consisting of R″ and a moiety targeting a target is utilized.

In some embodiments, provided technologies (e.g., compounds, compositions, methods, etc.) can modulate one or more properties and/or activities of various polypeptides. In some embodiments, the present disclosure provides various technologies that can inhibit activities of various polypeptides. In some embodiments, the present disclosure provides a method for inhibiting an activity of a polypeptide comprising a nucleophilic moiety, comprising contacting the polypeptide with a provided compound of a pharmaceutically acceptable salt thereof. In some embodiments, a nucleophilic moiety is —SH or —SeH or a salt form thereof. In some embodiments, the present disclosure provides a method for inhibiting an activity of a polypeptide comprising —SeH or a salt form thereof, comprising contacting the polypeptide with a provided compound of a pharmaceutically acceptable salt thereof. In some embodiments, the present disclosure provides a method for inhibiting an activity of apolypeptide comprising a selenocysteine residue, comprising contacting the polypeptide with a provided compound of a pharmaceutically acceptable salt thereof. In some embodiments, the present disclosure provides a method for inhibiting an activity of a polypeptide comprising —SH or a salt form thereof, comprising contacting the polypeptide with a provided compound of a pharmaceutically acceptable salt thereof. In some embodiments, the present disclosure provides a method for inhibiting an activity of a polypeptide comprising a cysteine residue, comprising contacting the polypeptide with a provided compound of a pharmaceutically acceptable salt thereof. In some embodiments, the present disclosure provides a method for inhibiting an activity of a polypeptide comprising —SeH or a salt form thereof in a system, comprising administering or delivering to the system a provided compound of a pharmaceutically acceptable salt thereof. In some embodiments, the present disclosure provides a method for inhibiting an activity of a polypeptide comprising a selenocysteine residue in a system, comprising administering or delivering to the system a provided compound of a pharmaceutically acceptable salt thereof. In some embodiments, the present disclosure provides a method for inhibiting an activity of a polypeptide comprising —SH or a salt form thereof in a system, comprising administering or delivering to the system a provided compound of a pharmaceutically acceptable salt thereof. In some embodiments, the present disclosure provides a method for inhibiting an activity of a polypeptide comprising a cysteine residue in a system, comprising administering or delivering to the system a provided compound of a pharmaceutically acceptable salt thereof. In some embodiments, a polypeptide comprises a characteristic portion of GPX4. In some embodiments, a polypeptide comprises a characteristic portion of GPX4 which characteristic portion comprises U73. In some embodiments, a polypeptide is GPX4.

In some embodiments, provided technologies are useful for modulating ferroptosis. In some embodiments, provided technologies are useful for inducing, promoting or enhancing ferroptosis. In some embodiments, the present disclosure provides a method for inducing, promoting or enhancing ferroptosis in a system, comprising administering or delivering to the system an effective amount of a provided compound or a pharmaceutically acceptable salt thereof. In some embodiments, the present disclosure provides a method for inducing, promoting or enhancing cell death in a system, comprising administering or delivering to the system an effective amount of a provided compound or a pharmaceutically acceptable salt thereof. In some embodiments, the present disclosure provides a method for inhibiting cell proliferation in a system, comprising administering or delivering to the system an effective amount of a provided compound or a pharmaceutically acceptable salt thereof.

In some embodiments, a system is an in vivo system. In some embodiments, a system is an in vitro system. In some embodiments, a system is or comprises a cell. In some embodiments, a system is or comprises a diseased cell, e.g., a cancer cell. In some embodiments, a system is or comprises a tissue. In some embodiments, a system is or comprises an organ. In some embodiments, a system is or comprises a sample. In some embodiments, a system is or comprises an organism. In some embodiments, a system is or comprises an animal. In some embodiments, a system is or comprises a subject. In some embodiments, a system is a human.

In some embodiments, the present disclosure provides a method for inducing or promoting cell death, comprising contacting the cell with a provided compound or a pharmaceutically acceptable salt thereof. In some embodiments, the present disclosure provides a method for inducing or promoting ferroptosis, comprising contacting a cell with a provided compound or a pharmaceutically acceptable salt thereof. In some embodiments, the present disclosure provides a method for inducing or promoting cell death, comprising administering or delivering to the cell an effective amount of a provided compound or a pharmaceutically acceptable salt thereof. In some embodiments, the present disclosure provides a method for inducing or promoting ferroptosis, comprising administering or delivering to a cell an effective amount of a provided compound or a pharmaceutically acceptable salt thereof.

In some embodiments, provided technologies are useful for preventing or treating various conditions, disorders or diseases. In some embodiments, the present disclosure provides a method for preventing a condition, disorder or disease, comprising administering or delivering to a subject susceptible thereto an effective amount of a compound or a pharmaceutically acceptable salt thereof. In some embodiments, the present disclosure provides a method for treating a condition, disorder or disease, comprising administering or delivering to a subject suffering therefrom an effective amount of a compound or a pharmaceutically acceptable salt thereof. In some embodiments, a condition, disorder or disease can be prevented or treated through increased ferroptosis. In some embodiments, a subject benefits from increased ferroptosis. In some embodiments, a condition, disorder or disease is a proliferative condition, disorder or disease. In some embodiments, a condition, disorder or disease is cancer. In some embodiments, a condition, disorder or disease is adrenocortical cancer, anal cancer, biliary cancer, bladder cancer, bone cancer, brain cancer, breast cancer, cervical cancer, colon cancer, endometrial cancer, esophageal cancer, head and neck cancer, intestinal cancer, liver cancer, lung cancer, oral cancer, ovarian cancer, pancreatic cancer, renal cancer, prostate cancer, salivary gland cancer, skin cancer, stomach cancer, testicular cancer, throat cancer, thyroid cancer, uterine cancer, vaginal cancer, sarcoma, or a soft tissue carcinoma. In some embodiments, a condition, disorder or disease is osteosarcoma, glioma, astrocytoma, neuroblastoma, cancer of the small intestine, bronchial cancer, small cell lung cancer, non-small cell lung cancer, basal cell carcinoma, or melanoma. In some embodiments, a condition, disorder or disease is hematologic cancer. In some embodiments, a condition, disorder or disease is acute lymphoblastic leukemia (ALL), acute myeloid leukemia (AML), lymphoma (e.g., Hodgkin's lymphoma, NonHodgkin's lymphoma, Burkitt's lymphoma), chronic lymphocytic leukemia (CLL), chronic myelogenous leukemia (CML), Hairy Cell chronic myelogenous leukemia (CML), or multiple myeloma. In some embodiments, a condition, disorder or disease is as described in WO 2014/011973, WO 2018/218087, WO 2019/168999, WO 2020/176757, WO 2021/041536, WO 2021/041539, WO 2021/183702, WO 2022/042657, Eaton J K, Furst L, Cai L L, Viswanathan V S, Schreiber S L., Structure-activity relationships of GPX4 inhibitor warheads, Bioorg Med Chem Lett. 2020 Dec. 1; 30(23):127538. doi: 10.1016/j.bmcl.2020.127538, Shimada K, Skouta R, Kaplan A, Yang W S, Hayano M, Dixon S J, Brown L M, Valenzuela C A, Wolpaw A J, Stockwell B R, Global survey of cell death mechanisms reveals metabolic regulation of ferroptosis, Nature Chemical Biology, 2016 May 9; 12: 497-503. doi: 10.1038/nchembio.2079, etc.

In some embodiments, a condition, disorder or disease associated with a polypeptide comprising —SeH or a salt form. In some embodiments, a condition, disorder or disease is associated with a polypeptide comprising a selenocysteine residue. In some embodiments, one or more properties and/or functions of a polypeptide is dependent on or is impacted by a selenocysteine residue. In some embodiments, a condition, disorder or disease is a GPX4-associated condition, disorder or disease.

### GPX4

In some embodiments, the present disclosure provides technologies that are useful for modulating properties and/or activities of GPX4. In some embodiments, provided technologies can inhibit GPX4. In some embodiments, provided technologies are useful for preventing or treating GPX4-associated conditions, disorders or diseases. In some embodiments, a provided compound can bond to a GPX4 selenocysteine residue, e.g., U73 of human GPX4 or an amino acid residue corresponding thereto. In some embodiments, a provided compound can selectively bond to a GPX4 selenocysteine residue over a GPX4 cysteine residue.

It has been reported that glutathione peroxidase 4 (GPX4) can reduce phospholipid hydroperoxide. Depletion of GPX4 has been reported to induce lipid peroxidation-dependent cell death. There are reports that cancer cells in various cases including in drug-induced and/or therapy-resistant states may have enhanced dependence of GPX4 to prevent ferroptotic cell death. Certain studies have reported that lipophilic antioxidants, such as ferrostatin, can rescue cells from GPX4 inhibition-induced ferroptosis. In some embodiments, it has been reported that certain GPX4-knockout cells can survive in the presence of ferrostatin, however, when ferrostatin is absent, these cells undergo ferroptosis (see, e.g., Viswanathan et al., Nature 547:453-7, 2017). It has also been reported that GPX4 inhibition may be rescued by blocking other components in ferroptosis pathways, such as lipid ROS scavengers (ferrostatin, liproxstatin, etc.), lipoxygenase inhibitors, iron chelators, caspase inhibitors, etc., which an apoptotic inhibitor typically does not rescue. In some embodiments, provided compounds induce or promote death of diseased cells, e.g., cancer cells. In some embodiments, provided compounds can be useful to induce ferroptotic cancer cell death. In some embodiments, the present disclosure provides technologies for treating various conditions, disorders or diseases, e.g., cancer, that are dependent on GPX4, e.g., its activity for reducing phospholipid hydroperoxide. In some embodiments, a subject benefits from inhibition of GPX4.

In some embodiments, various moieties, e.g., isourea or isothiourea moieties, may be utilized as leaving groups for various reactions. In some embodiments, a compound reacts with an electrophile, e.g., selenocysteine or a salt thereof, or a selenocysteine residue in a polypeptide, e.g., U73 of GPX4. In some embodiments, —SeH, e.g., of a selenocysteine or a salt form thereof replaces an isourea or isothiourea moiety. In some embodiments, it replaces -T-C(═NR^{w1})N(R^{w2})(R^{w3}) wherein each variable is independently as described herein. In some embodiments, a compound reacts with cysteine or a salt thereof, or a cysteine residue in a polypeptide. In some embodiments, —SH, e.g., of a cysteine or a salt form thereof replaces an isourea or isothiourea moiety. In some embodiments, it replaces -T-C(═NR^{w1})N(R^{w2})(R^{w3}) wherein each variable is independently as described herein.

In some embodiments, a reaction is useful for delivering an agent to a target (e.g., apolypeptide, a nucleic acid, a cell, a tissue, an organ, a sample, etc.). In some embodiments, the present disclosure provides a compound having the structure of [PAYLOAD]-R⁴ or a salt thereof, wherein [PAYLOAD] is an agent to be delivered, and R⁴ is as described herein. In some embodiments, [PAYLOAD] is or comprises one or more small molecule, nucleic acid, polypeptide, lipid and/or carbohydrate moieties. In some embodiments, [PAYLOAD] is or comprises a small molecule moiety. In some embodiments, [PAYLOAD] is or comprises a nucleic acid moiety. In some embodiments, [PAYLOAD] is or comprises a polypeptide moiety. In some embodiments, [PAYLOAD] is or comprises a lipid moiety. In some embodiments, [PAYLOAD] is or comprises a carbohydrate moiety. In some embodiments, [PAYLOAD] is or comprises a reactive moiety. In some embodiments, [PAYLOAD] is or comprises a detectable moiety. In some embodiments, [PAYLOAD] is or comprises a label moiety. In some embodiments, [PAYLOAD] is or comprises a detectable moiety. Various detectable and/or label moieties are described in the art and may be in accordance with the present disclosure. For example, in some embodiments, it is or comprises a fluorescent moiety. In some embodiments, it is or comprises a radioactive moiety. In some embodiments, a provided moiety has selectivity for certain reactive groups of a target over others. In some embodiments, certain groups of a target are selectively targeted and/or labeled over others. In some embodiments, in a compound being delivered, R⁴ is -L^{w}-R^{wh} wherein L^{w} and R^{wh} are independently as described herein.

In some embodiments, the present disclosure provides technologies for delivering an agent to a target (e.g., a polypeptide, a nucleic acid, a cell, a tissue, an organ, a sample, etc.). In some embodiments, a delivered agent is a urea or thiourea compound. In some embodiments, a delivered agent is a compound having the structure of T=C(NHR^{w1})N(R^{w2})(R^{w5}) or a salt thereof, wherein each variable is independently as described herein. In some embodiments, for delivery of such a compound, a useful compound comprises a targeting moiety toward a target, and an isourea or isothiourea moieties such as R⁴ as described herein. In some embodiments, a targeting moiety is a small molecule moiety, e.g., a binder or inhibitor of a target. In some embodiments, a targeting moiety is an antibody toward a target, e.g., an antibody targeting a polypeptide target. In some embodiments, a compound is enriched at a target location, and reacts with an nucleophile to release a compound to be delivered, e.g., a urea, a thiourea, a compound having the structure of T=C(NHR^{w1})N(R^{w2})(R^{w3}) or a salt thereof, etc. In some embodiments, a target site is or comprises an nucleophile. In some embodiments, a target site is or comprises a higher level (e.g., about or at least about 1-1000, 1-500, 1-200, 1-100, 1-50, 1-20, 1-10, 2, 3, 4, 5, 6, 7, 8, 9, 10, etc. fold) of an nucleophile compared to a non-target site. In some embodiments, an nucleophile comprises —SeH or a salt form thereof. In some embodiments, an nucleophile is or comprises a selenocysteine residue.

In some embodiments, the present disclosure provides a method, comprising:
1) contacting a compound, e.g., of formula A, B, C, D, E, F, G, H, etc., or a salt thereof with a nucleophile moiety; and
2) loading a payload to a target.

In some embodiments, the present disclosure provides a method, comprising:
1) contacting a compound, e.g., of formula A, B, C, D, E, F, G, H, etc., or a salt thereof with a nucleophile moiety; and
2) generating a compound having the structure of T=C(NHR^{w1})N(R^{w2})(R^{w3}) or salt thereof, wherein each variable is independently as described herein.

In some embodiments, a compound has the structure of [PAYLOAD]-R⁴ or a salt thereof. In some embodiments, a method comprises loading a payload to a nucleophile moiety and generating a compound having the structure of T=C(NHR^{w1})N(R^{w2})(R^{w3}) or salt thereof. In some embodiments, a payload is [PAYLOAD] as described herein. In some embodiments, a target is or comprises a nucleophile moiety, e.g., —SeH, —SH, or a salt form thereof. In some embodiments, a target is or comprises a polypeptide which comprises a selenocysteine residue. In some embodiments, a target is or comprises a polypeptide which comprises a cysteine residue. In some embodiments, a nucleophile moiety is in a polypeptide. In some embodiments, a nucleophile moiety is or comprises —OH. In some embodiments, a nucleophile moiety is or comprises —SH. In some embodiments, a nucleophile moiety is or comprises —SeH. In some embodiments, a —OH, —SH or —SeH group is in a salt form. In some embodiments, a nucleophile moiety is in a polypeptide. In some embodiments, a nucleophile moiety is in a side chain of amino acid residue in a polypeptide. In some embodiments, a nucleophile moiety is —SH of a cysteine residue in a polypeptide. In some embodiments, a nucleophile moiety is —SeH of a selenocysteine residue in a polypeptide.

In some embodiments, a use is described in WO 2014/011973, WO 2018/218087, WO 2019/168999, WO 2020/176757, WO 2021/041536, WO 2021/041539, WO 2021/183702, WO 2022/042657, Eaton J K, Furst L, Cai L L, Viswanathan V S, Schreiber S L., Structure-activity relationships of GPX4 inhibitor warheads, Bioorg Med Chem Lett. 2020 Dec. 1; 30(23):127538. doi: 10.1016/j.bmcl.2020.127538, Shimada K, Skouta R, Kaplan A, Yang W S, Hayano M, Dixon S J, Brown L M, Valenzuela C A, Wolpaw A J, Stockwell B R, Global survey of cell death mechanisms reveals metabolic regulation of ferroptosis, Nature Chemical Biology, 2016 May 9; 12: 497-503. doi: 10.1038/nchembio.2079, etc.

### Pharmaceutical Compositions and Administration

In some embodiments, the present disclosure provides a pharmaceutical composition that comprises a provided compound or a pharmaceutically acceptable salt thereof, and a pharmaceutically acceptable carrier. In some embodiments, the present disclosure provides a pharmaceutical composition that delivers a provided compound or a pharmaceutically acceptable salt thereof, and comprises a pharmaceutically acceptable carrier.

In some embodiments, compounds in pharmaceutical compositions are provided as pharmaceutically acceptable salts. In some embodiments, salts can be formed with many acids, e.g., hydrochloric, sulfuric, acetic, lactic, tartaric, malic, succinic, benzenesulfonic, etc. In some embodiments, salts can be formed with bases. In some embodiments, salts are alkali, alkaline earth metal, or ammonium salts, e.g., sodium, calcium, diethanolamine, ethanolamine, trialkylamine salts, etc. In some embodiments, salts are more soluble in aqueous or other protonic solvents than corresponding free acid or base forms.

In some embodiments, pharmaceutically acceptable salt is a solid, e.g., a tablet, power, etc. In some embodiments, a pharmaceutical composition may be a lyophilized powder. In some embodiments, pharmaceutical compositions or formulations are tablets (coated or uncoated), capsules (hard or soft), microbeads, powder, granules and/or crystals.

Pharmaceutical compositions can include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. In some embodiments, a pharmaceutically acceptable salt is a liquid. In some embodiments, pharmaceutically acceptable carriers include liquids such as water, saline, glycerol, sugars and ethanol. Additionally, auxiliary substances, such as wetting or emulsifying agents, pH buffering substances, and the like, may be present in such vehicles. In some embodiments, a pharmaceutical composition comprises a provided compound or a pharmaceutically acceptable salt thereof dissolved in a pharmaceutically acceptable buffer. In some embodiments, a buffer is a saline buffer. In some embodiments, a buffer has a pH around 7.4. In some embodiments, a pharmaceutically acceptable salt is a gel, suspension, or ointment.

Supplementary active compounds (e.g., preservatives, antibacterial, antiviral and antifungal agents) can also be incorporated into pharmaceutical compositions.

Various technologies, e.g., routes, modes, dosage regimens, etc. may be utilized to administer and/or deliver provided compounds and compositions in accordance with the present disclosure. In some embodiments, a route and/or mode of administration can vary depending upon desired results. One with skill in the art, i.e., a physician, is aware that dosage regimens can be adjusted to provide a desired response, e.g., a therapeutic response. In some embodiments, a method of administration is intradermal, intramuscular, intraperitoneal, intravenous, subcutaneous, intranasal, epidural, oral, sublingual, intracerebral, intrathecal, intravaginal, transdermal, rectal, by inhalation, or topical, particularly to the ears, nose, eyes, or skin. In some embodiments, a mode of administration is left to discretion of a practitioner. Pharmaceutical compositions can be formulated to be compatible with a particular route of administration or delivery as set forth herein or known to one of skill in the art. In some embodiments, methods and uses of the present disclosure include delivery and administration systemically, regionally or locally, or by any suitable route, for example, by injection or infusion or orally. In some embodiments, delivery of a pharmaceutical composition in vivo may generally be accomplished via injection using a conventional syringe, although other delivery methods such as convection-enhanced delivery can also be used (see, e.g., U.S. Pat. No. 5,720,720). In some embodiments, compounds and compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. In some embodiments, modes of administration include oral and pulmonary administration, suppositories, and transdermal applications. In some embodiments, a compound or composition is administered orally. In some embodiments, a compound or composition is administered intravenously.

In some embodiments, provided compositions are suitable for parenteral administration. In some embodiments, such compositions comprise aqueous and non-aqueous solutions, suspensions or emulsions of active compounds, which preparations are typically sterile and can be isotonic with blood of intended recipients. Some examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran. Additionally, suspensions of active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, a suspension may also contain suitable stabilizers or agents which increase solubility to allow for the preparation of highly concentrated solutions.

Co-solvents and adjuvants may be added to compositions and formulations. Some examples of co-solvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling can include amount, frequency, and method of administration.

Certain useful technologies for formulating pharmaceutical compositions and delivery systems appropriate for compounds, compositions, methods and uses of the present disclosure are described in e.g., Remington: The Science and Practice of Pharmacy. 21st Edition. Philadelphia, PA. Lippincott Williams & Wilkins, 2005, and can be utilized in accordance with the present disclosure.

In some embodiments, the present disclosure provides methods for delivering provided compounds and compositions into cells, animals or subjects. In some embodiments, such methods include contacting a system (e.g., a cell or tissue of a subject) with, or administering or delivering to a system (e.g., a subject such as a mammal or human) a provided compound, e.g., a compound of formula A, B, C, D, E, F, G, H or a salt thereof, or a composition thereof.

A compound or composition described herein can be administered in a sufficient or effective amount to a subject (or a cell, tissue or organ thereof) in need thereof. Doses can vary and may depend upon type, onset, progression, severity, frequency, duration, or probability of a condition, disorder or disease to which treatment or prevention is directed, a clinical endpoint desired, previous or simultaneous treatments, general health, age, gender, race, immunological competency, etc. of a subject and other factors that will be appreciated by a skilled artisan. Dose amount, number, frequency or duration may be increased or reduced, as indicated by efficacy, any adverse side effects, complications or other risk factors of a treatment or therapy and the status of a subject. A skilled artisan will appreciate factors that may influence dosage and timing required to provide an amount sufficient for providing a therapeutic or prophylactic benefit. A dose or dosage regimen to achieve a therapeutic effect may vary based on several factors including route of administration, amount to achieve a therapeutic effect, specific condition, disorder or disease treated, any host immune response to administered compound or composition, stability of administered compound or composition, etc.

An effective amount or a sufficient amount can be provided in a single administration, may require multiple administrations, and, can be, administered alone or in combination with another composition (e.g., comprising or delivering another therapeutic agent). For example, an amount may be increased as indicated by the need of a subject, type, status and severity of a condition, disorder or disease treated and/or side effects (if any) of treatment. In some embodiments, amounts considered effective include amounts that result in a reduction of the use of, improvement of efficacy of, and/or reduction of side effects of another treatment, therapeutic regimen or protocol.

In some embodiments, a compound or composition is utilized in combination with another therapy. For example, for treating cancer, a compound or composition may be utilized in combination with another cancer therapy. In some embodiments, such another therapy is a cancer therapy. In some embodiments, it is or comprises surgery. In some embodiments, it is or comprises radiation therapy. In some embodiments, it is or comprises chemotherapy. In some embodiments, it is or comprises immunotherapy. In some embodiments, another therapy is or comprises a therapeutic agent. In some embodiments, a compound or composition is utilized in combination with another therapeutic agent. In some embodiments, a therapeutic agent is a drug, e.g., a cytotoxic agent, a chemotherapeutic agent, a radiation therapeutic agent, an immunotherapy agent, etc. In some embodiments, it is or comprises an antibody agent. In some embodiments, it is an immune checkpoint inhibitor. In some embodiments, it is a PDl antibody agent. In some embodiments, it is a PD-L1 antibody agent. In some embodiments, a therapeutic agent helps to control, reduce or manage one or more side effects.

Among other things, the present disclosure provides the following Embodiments:

### EXEMPLIFICATION

Certain examples of provided technologies (e.g., compounds, compositions, methods (methods of preparation, use, assessment, etc.), etc.) are described herein. Those skilled in the art reading the present disclosure appreciate that various technologies, including those described below and modifications, variants and derivatives thereof, are available for manufacturing, characterizing and/or assessing provided technologies in accordance with the present disclosure.

In some embodiments, an Example may utilize one or more of the following abbreviation:
EA (EtOAc): ethyl acetate;
ACN(MeCN): acetonitrile;
DCM: dichloromethane;
THF: tetrahydrofuran;
Ac: acetyl;
AcOH: acetic acid;
n-Bu: n-butyl;
Et: ethyl;
DMAP: 4-dimethylaminopyridine;
TEA(Et₃N): triethylamine;
DIEA(DIPEA): diisopropylethylamine;
T₃P: propyl phosphate tricyclic anhydride solution;
Tf₂O: trifluoromethanesulfonic anhydride;
DCC: dicyclohexylcarbodiimide;
DCE: 1,2-dichloroethane;
i-PrOH(PrOH, iPrOH): isopropyl alcohol;
EDC: 1-(3-dimethylaminopropyl)-3-ethylcarbodiimide hydrochloride;
DIC: N,N′-diisopropylcarbodiimide;
Cu(OTf)₂: Copper(II) trifluoromethanesulfonate;
DME: 1,2-dimethoxy-ethan;
STAB: sodium triacetoxyborohydride;
TsOH: p-toluenesulfonic acid;
DBE: dibasic esters;
NFSI: N-Fluorobenzenesulfonimide;
[PdCl(allyl)]₂: allylpalladium chloride dimer;
Dtbpf 1,1′-bis(di-tert-butylphosphino)ferrocene;
4A MS (4As, 4A MS, 4A Molecular Sieve): 4A Molecular Sieve;
Pin₂B2/(PinB)₂: bis(pinacolato)diboron;
Bu₃SnCH₂OTBS: tert-butyl-dimethyl-(tributylstannylmethoxy)silane
DAST: diethylaminosulfur trifluoride;
t-BuOK: Potassium tert-butoxide;
CAN: Ceric ammonium nitrate
HATU: O-(7-Azabenzotriazol-1-yl)-N,N,N′,N′-tetraMethyluroniuM hexafluorophosphate;
Pd (dppf)₂Cl₂: [1,1′-Bis(diphenylphosphino)ferrocene]dichloropalladium(II)
Ac₂O: acetic anhydride;
PhMe: toluene;
BMS (BH₃.Me₂S): borane-methyl sulfide complex;
DIAD: Diisopropyl azodicarboxylate;

### Example 1. Preparation of Compound Int. 1

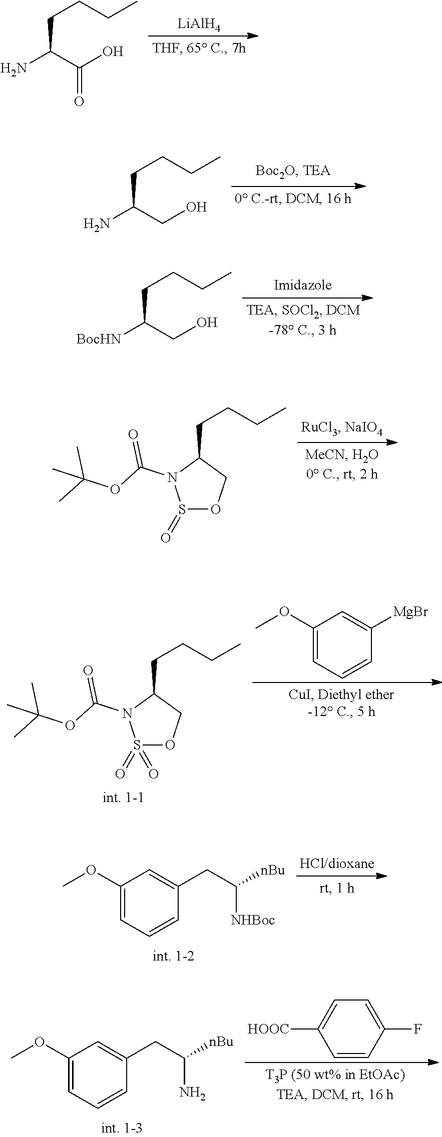

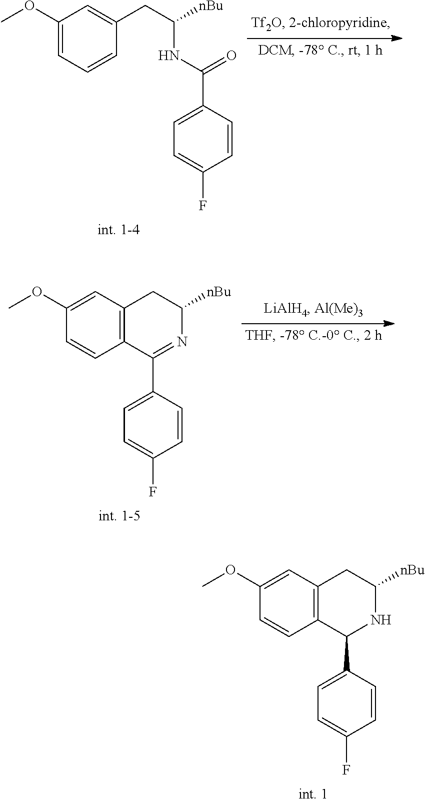

1. Synthesis of compound (S)-2-aminohexan-1-ol: A solution of (S)-2-aminohexanoic acid (5.0 g, 38.1 mmol) in 20 mL of THF was added LiAlH₄ (76.3 mL, 76.2 mmol) at 0° C. and stirred for 1 h, then heated to 65° C. and stirred for 7 h. The reaction was quenched with NH₄Cl aqueous solution and extracted with ethyl acetate (100 mL×3). The organic phases were concentrated under reduced pressure, then the residue was purified by column chromatography to afford the title compound (4.5 g). ¹H NMR (400 MHz, Chloroform-d) δ 3.60 (dd, J=10.6, 4.0 Hz, 1H), 3.29 (dd, J=10.6, 8.0 Hz, 1H), 2.84 (tt, J=8.4, 4.4 Hz, 1H), 1.48-1.31 (m, 6H), 0.96-0.91 (m, 3H).

2. Synthesis of compound tert-butyl (S)-(1-hydroxyhexan-2-yl)carbamate. A solution of (S)-2-aminohexan-1-ol (1.0 g, 8.5 mmol) in 5 mL of DCM was added TEA (1.7 g, 17.1 mmol) and Boc₂O (2.2 g, 10.2 mmol) at 0° C. and stirred for 1 h, then stirred at room temperature for 16 h. The reaction was diluted with H₂O (50 mL) and extracted with DCM (30 mL×3). The organic phases was removed under reduced pressure then the residue was purified by column chromatography to afford the title compound (1.2 g). ¹H NMR (400 MHz, Chloroform-d) δ 4.61 (s, 1H), 3.69-3.48 (m, 3H), 2.50 (s, 1H), 1.70 (s, 1H), 1.36-1.25 (m, 6H), 0.90 (td, J=5.6, 4.8, 2.4 Hz, 3H).

3. Synthesis of compound tert-butyl (4S)-4-butyl-1,2,3-oxathiazolidine-3-carboxylate 2-oxide. A solution of tert-butyl (S)-(1-hydroxyhexan-2-yl)carbamate (3.8 g, 17.5 mmol), imidazole (4.8 g, 70.0 mmol) and TEA (25.3 g, 52.5 mmol) in DCM (50 mL) was added SOCl₂ (2.3 g, 19.3 mmol) at −78° C. and stirred for 3 h. The reaction was quenched with NH₄Cl aqueous and extracted with DCM (80 mL×3). The solvent was removed under reduced pressure to afford the title compound (4.0 g).

4. Synthesis of compound int. 1-1. A solution of tert-butyl (4S)-4-butyl-1,2,3-oxathiazolidine-3-carboxylate 2-oxide (4.0 g, 15.2 mmol), RuCl₃ (23.9 mg, 0.11 mmol) in MeCN (200 mL) and H₂O (200 mL) was added NaIO₄ (3.6 g, 16.7 mmol) at 0° C. and then stirred at room temperature for 2 h. The solvent was removed under reduced pressure, then the residue was purified by column chromatography to afford the title compound (2.5 g). ¹H NMR (400 MHz, Chloroform-d) δ 4.70-4.60 (m, 1H), 4.40-4.26 (m, 2H), 1.98-1.75 (m, 2H), 1.57 (s, 9H), 1.47-1.24 (m, 6H), 0.94 (t, J=7.0 Hz, 3H).

5. Synthesis of compound int. 1-2. To a solution of CuI (27.2 mg, 0.14 mmol) dissolved in 20 mL of diethyl ether was added (3-methoxyphenyl)magnesium bromide (3 mL) dropwise at −78° C. and stirred for 30 min. Another solution of tert-butyl (S)-4-butyl-1,2,3-oxathiazolidine-3-carboxylate 2,2-dioxide (400 mg, 1.43 mmol) in 20 mL of diethyl ether was added dropwise at −12° C. and stirred for 5 h. The solvent was removed under reduced pressure, then the residue was purified by column chromatography to afford the title compound (165 mg). ¹H NMR (400 MHz, Chloroform-d) δ 7.19 (t, J=7.8 Hz, 1H), 6.82-6.69 (m, 3H), 4.30 (d, J=8.4 Hz, 1H), 3.79 (s, 4H), 2.73 (d, J=6.8 Hz, 2H), 1.49 (s, 3H), 1.41 (s, 9H), 0.92-0.83 (m, 4H).

6. Synthesis of compound int. 1-3. A solution of tert-butyl (S)-(1-(3-methoxyphenyl)hexan-2-yl)carbamate (150 mg, 0.5 mmol) in 3 mL of HCl-1,4-dioxane (4M) and stirred at room temperature for 1 h. The solvent was removed under reduced pressure, then the residue was purified by column chromatography to afford title compound (100 mg). ¹H NMR (400 MHz, Chloroform-d) δ 8.52 (s, 2H), 7.34-7.28 (m, 1H), 6.96-6.83 (m, 3H), 3.87 (s, 3H), 3.54 (s, 1H), 3.29 (d, J=12.8 Hz, 1H), 3.00 (s, 1H), 1.78 (s, 6H), 0.94 (t, J=7.2 Hz, 3H).

7. Synthesis of compound int. 1-4. A solution of 4-fluorobenzoic acid (3.3 g, 24.1 mmol), triethanolamine (14.4 g, 96.5 mmol) in dichloromethane (500 mL) was stirred at 0° C. for 15 min before T₃P (11.5 g, 36.2 mmol) was added and stirred for another 5 min, then added 1-(3-methoxyphenyl)hexan-2-amine (5.0 g, 24.1 mmol) at room temperature and stirred for 16 h. The solvent was removed under reduced pressure, then the residue was purified by column chromatography to afford the title compound (3.8 g). ¹H NMR (400 MHz, Chloroform-d) δ 4.70-4.60 (m, 1H), 4.40-4.26 (m, 2H), 1.98-1.75 (m, 2H), 1.57 (s, 9H), 1.47-1.24 (m, 6H), 0.94 (t, J=7.2 Hz, 3H).

8. Synthesis of compound int. 1-5. To a stirred solution of 4-fluoro-N-[(2S)-1-(3-methoxyphenyl)hexan-2-yl]benzamide (9.0 g, 27.3 mmol) in dichloromethane (1 L) was added Tf₂O (15.4 g, 54.64 mmol) and stirred at −78° C. for 5 min before 2-chloropyridine (6.2 g, 54.6 mmol)) was added at 0° C. The mixture was stirred at 15° C. for 1 h. The solvent was removed under reduced pressure, then the residue was purified by column chromatography to afford the title compound (7.5 g). ¹H NMR (400 MHz, Chloroform-d) δ 7.19 (t, J=7.6 Hz, 1H), 6.82-6.69 (m, 3H), 4.30 (d, J=8.4 Hz, 11H), 3.79 (s, 4H), 2.73 (d, J=6.8 Hz, 2H), 1.49 (s, 3H), 1.41 (s, 9H), 0.92-0.83 (m, 4H).

9. Synthesis of compound int. 1. To a stirred solution of (3S)-3-butyl-1-(4-fluorophenyl)-6-methoxy-3,4-dihydroisoquinoline (3.0 g, 9.63 mmol) in 150 mL of THF was added LiAlH₄ (3.7 g, 96.3 mmol) and AlMe₃ (3.5 g, 48.2 mmol) at −78° C. and stirred for 1 h, then warmed the solution to 0° C. and stirred for 2 h. The solvent was removed under reduced pressure, then the residue was purified by column chromatography and recrystallization by n-hexane (20 mL) to afford the title compound (550 mg). ¹H NMR (400 MHz, Chloroform-d) δ 7.18-7.10 (m, 2H), 7.04-6.95 (m, 2H), 6.86-6.81 (m, 1H), 6.74-6.69 (m, 2H), 5.20 (s, 1H), 3.83 (s, 3H), 2.97-2.85 (m, 2H), 2.58 (dd, J=16.8, 10.2 Hz, 1H), 1.48-1.41 (m, 2H), 1.29 (td, J=7.2, 6.8, 3.6 Hz, 5H), 0.89 (t, J=6.8 Hz, 3H).

### Example 2. Preparation of Compound Int. 2

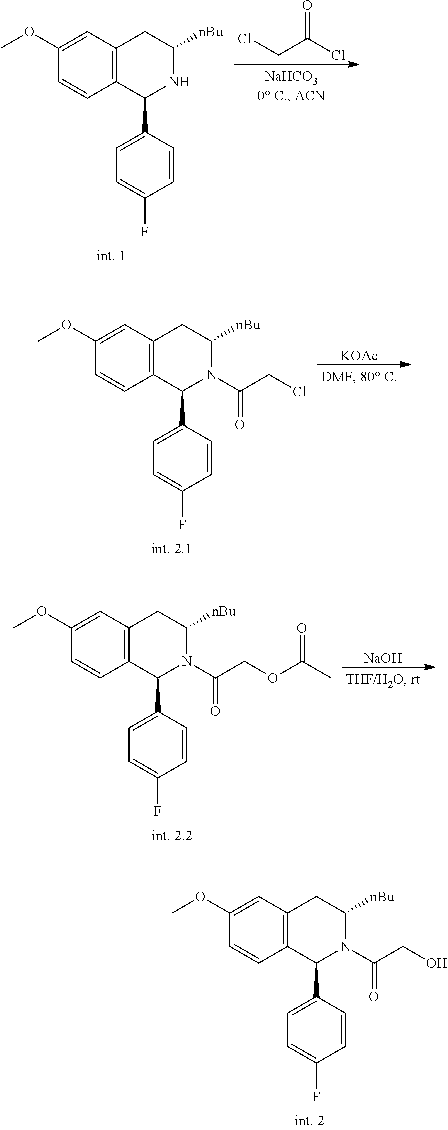

1. Synthesis of compound int. 2-1. A solution of (1S,3S)-3-butyl-1-(4-fluorophenyl)-6-methoxy-1,2,3,4-tetrahydroisoquinoline (30 mg, 0.1 mmol) in 2 mL of ACN was added NaHCO₃ (63 mg, 0.75 mmol) and stirred for 30 min at 0° C. and then added 2-chloroacetyl chloride (5.65 mg, 0.15 mmol) and stirred for 1 h. The solvent was removed under reduced pressure then the residue was purified by column chromatography to afford the title compound (22 mg). ¹H NMR (400 MHz, Chloroform-d) δ 9.56 (d, J=8.4 Hz, 1H), 9.49 (dt, J=10.8, 5.6 Hz, 2H), 9.33-9.13 (m, 3H), 9.05 (d, J=2.4 Hz, 1H), 8.60 (d, J=42.8 Hz, 1H), 7.07-6.82 (m, 1H), 6.16 (dd, J=5.6, 1.2 Hz, 3H), 5.52-5.42 (m, 1H), 5.31-5.03 (m, 2H), 4.03 (d, J=25.2 Hz, 1H), 3.59 (d, J=11.2 Hz, 5H), 3.20 (q, J=7.2 Hz, 3H).

2. Synthesis of compound int. 2-2. A solution of 1-((1S,3S)-3-butyl-1-(4-fluorophenyl)-6-methoxy-3,4-dihydroisoquinolin-2(1H) -yl)-2-chloroethan-1-one (10 mg, 0.026 mmol) in 2 mL of DMF was added KOAc (12.6 mg, 0.13 mmol) and stirred for 30 min at 80° C. The solvent was removed under reduced pressure then the residue was purified by column chromatography to afford the title compound (12 mg).

3. Synthesis of compound int. 2. A solution of (S)-2-(1-(4-fluorophenyl)-6-methoxy-3,4-dihydroisoquinolin-2(1H)-yl)-M2-oxoethyl acetate (320 mg, 0.78 mmol) in 3 mL of THF and 1 mL of H₂O was added NaOH (62.4 mg, 1.56 mmol) and stirred for 30 min at room temperature. The solvent was removed under reduced pressure, then the residue was purified by column chromatography to afford the title compound (300 mg). ¹H NMR (400 MHz, Chloroform-d) δ 7.36-7.27 (m, 1H), 7.24-7.10 (m, 2H), 7.08-6.88 (m, 2H), 6.86-6.76 (m, 1H), 6.68 (s, 1H), 5.66 (d, J=96.5 Hz, 1H), 4.69 (d, J=18.6 Hz, 1H), 4.42-4.22 (m, 1H), 4.01-3.86 (m, 1H), 3.77 (d, J=19.6 Hz, 5H), 3.62 (t, J=4.4 Hz, 1H), 3.18-2.68 (m, 2H), 1.94-1.81 (m, 2H), 1.64 (s, 2H), 0.86 (t, J=6.4 Hz, 4H).

### Example 3. Preparation of Compound Int. 3

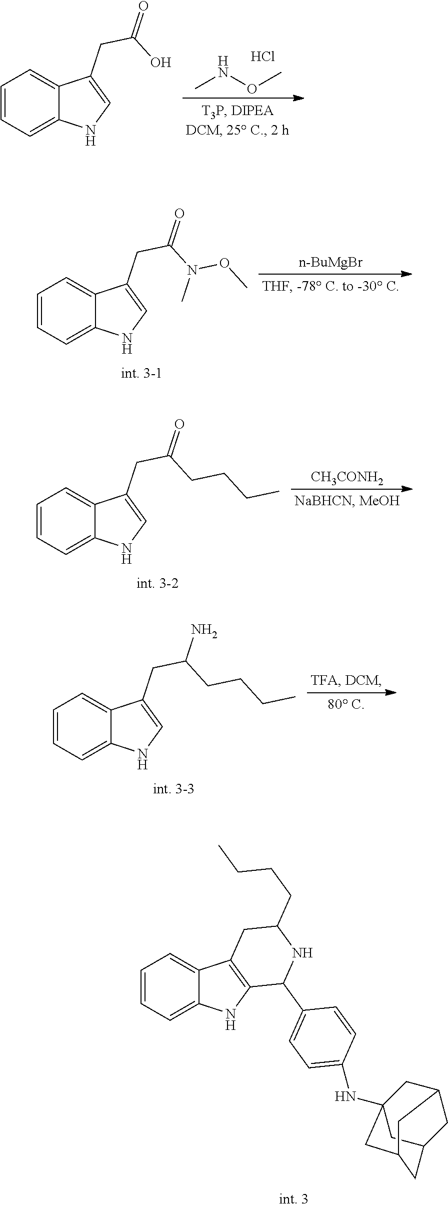

1. Synthesis of compound int. 3-1. A mixture of 2-(1H-indol-3-yl)acetic acid (20.0 g, 114 mmol), N,O-dimethylhydroxylamine hydrochloride (12.2 g, 126 mmol), T₃P (108 g, 170 mmol, 50% ), DIPEA (40.1 g, 311 mmol) in DCM (100 mL) was stirred at 25° C. for 2 h. The reaction solution was concentrated in vacuo and the crude was purified by silica gel column to get the desired product (22 g). ESI-MS (EI⁺, m/z): 219.15.

2. Synthesis of compound int. 3-2. To a solution of 2-(1H-indol-3-yl)-N-methoxy-N-methylacetamide (660 mg, 3.02 mmol) in THF (5 mL) was added bromo(butyl)magnesium (1.21 g, 7.5 mmol) dropwise at −78° C. and stirred at −30° C. for 3 h. The solution was added NH₄Cl(aq.) at −78° C. and the mixture was extracted with EtOAc (50 mL×3). The combined organic layers were dried and concentrated. The residue was purified by silica gel column to get title compound (500 mg).

3. Synthesis of compound int. 3-3. To a solution of 1-(1H-indol-3-yl)hexan-2-one (1.50 g, 6.97 mmol), ammonium acetate (8.06 g, 105 mmol), sodium cyanoboranuide (875 mg, 13.9 mmol) in MeOH (30 mL) was stirred at 25° C. for 16 h. The reaction was concentrated in vacuo and the residue was purified by column to get title compound (1.1 g). ESI-MS (EI⁺, m/z): 217.2.

4. Synthesis of compound int. 3. A mixture of 4-[(adamantan-1-yl)amino]benzaldehyde (519 mg, 2.03 mmol), 1-(1H-indol-3-yl)hexan-2-amine (400 mg, 1.85 mmol), trifluoroacetic acid (422 mg, 3.7 mmol) in DCE (5 mL) was stirred at 80° C. for 12 h. The reaction solution was concentrated in vacuo and the residue was purified by silica gel column to get title compound (240 mg). ¹H NMR (400 MHz, DMSO-d₆) δ 10.13 (s, 1H), 7.40-7.34 (m, 1H), 7.20 (d, J=8.0 Hz, 1H), 7.00 (d, J=8.4 Hz, 2H), 6.93 (ddd, J=10.2, 7.2, 1.2 Hz, 2H), 6.73 (d, J=8.4 Hz, 2H), 4.95 (d, J=2.0 Hz, 1H), 2.90 (d, J=8.4 Hz, 1H), 2.79 2.70 (m, 1H), 2.42-2.31 (m, 1H), 2.06 (t, J=4.4 Hz, 3H), 1.87 (d, J=2.8 Hz, 6H), 1.64 (d, J=2.8 Hz, 7H), 1.57-1.22 (m, 7H), 0.91 (t, J=7.2 Hz, 3H).

### Example 4. Preparation of Compound Int. 4

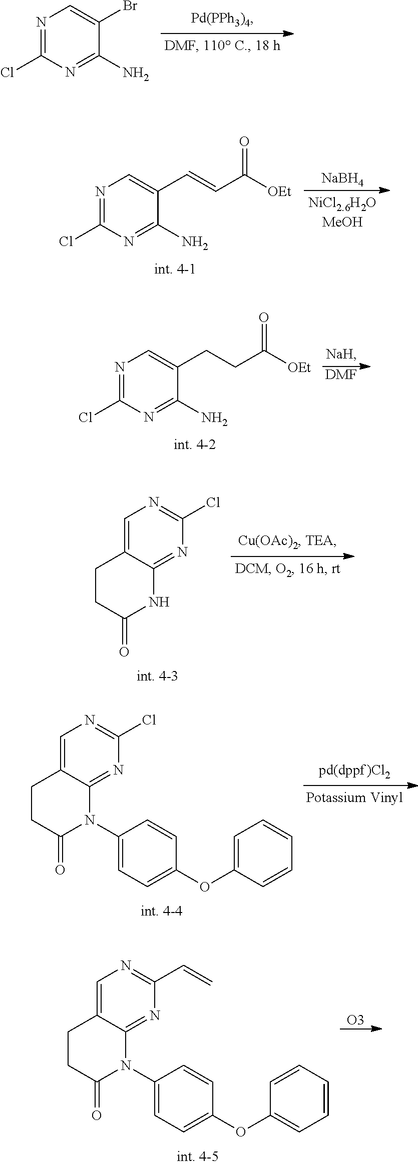

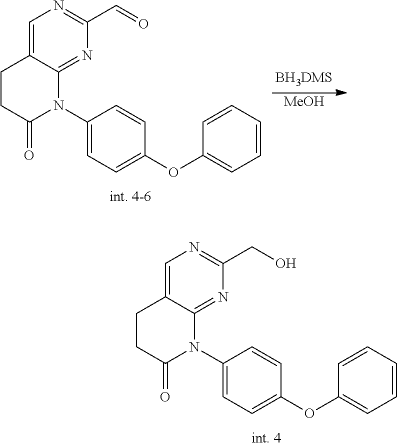 [SMILES: "MeOH" ; O=Cc1ncc2c(n1)N(c1ccc(Oc3ccccc3)cc1)C(=O)CC2 ; O=C1CCc2cnc(CO)nc2N1c1ccc(Oc2ccccc2)cc1]

1. Synthesis of compound int. 4-1. To a solution of 5-bromo-2-chloropyrimidin-4-amine (20.0 g, 96.0 mmol) and ethyl acrylate (48.0 g, 480 mmol) was added Pd₂(PPh₃)₄ (11.1 g, 9.60 mmol), the mixture was stirred at 110° C. for 18 h under N₂ atmosphere. The mixture was concentrated. The crude material was purified by column chromatography to afford the title product (2.0 g). ESI-MS (EI+, m/z): 228.15.

2. Synthesis of compound int. 4-2. To a solution of ethyl (2E)-3-(4-amino-2-chloropyrimidin-5-yl)prop-2-enoate (2.00 g, 8.79 mmol) and NiCl₂·6H₂O (209 mg, 0.88 mmol) in MeOH (20 mL) was added sodium borohydride (997 mg, 26.4 mmol) and the mixture was stirred at 0° C. for 8 h. The mixture was concentrated under reduced pressure. The crude material was purified by column chromatography to afford the title product (1.10 g). ESI-MS (EI+, m/z): 229.66.

3. Synthesis of compound int. 4-3. To a solution of ethyl 3-(4-amino-2-chloropyrimidin-5-yl)propanoate (300 mg, 1.31 mmol) in DMF (5 mL) was added NaH (125 mg, 5.23 mmol) at room temperature under a N₂ atmosphere and the mixture was stirred for 1 h. The mixture was concentrated. The crude material was purified by column chromatography to get the title product (150 mg). ESI-MS (EI⁺, m/z): 184.05.

4. Synthesis of compound int. 4-4. To a solution of 2-chloro-5H, 6H, 7H, 8H-pyrido[2,3-d]pyrimidin-7-one (800 mg, 4.36 mmol), copper(II) acetate (613 mg, 4.36 mmol), 4-phenoxyphenylboronic acid (1.87 g, 8.71 mmol) and TEA (1.32 g, 13.1 mmol) in DCM (10 mL) was added 4A molecular sieve (200 mg), then the mixture was stirred at room temperature for 16 h under a O₂ atmosphere. The mixture was centrifugal, the liquid was concentrated to afford title product (400 mg). ESI-MS (EI+, m/z): 352.06.

5. Synthesis of compound int. 4-5. To a solution of 2-chloro-8-(4-phenoxyphenyl)-5,8-dihydropyrido[2,3-d]pyrimidin-7(6H)-one (600 mg, 1.71 mmol) in dioxane (10 mL) and H₂O (2 mL) at room temperature was added potassium trifluoro(vinyl)borate (914 mg, 6.82 mmol), N-methyldicyclohexylamine (1.00 g, 5.12 mmol), Pd(dppf)Cl₂ (125 mg, 0.17 mmol) and the mixture was stirred at 100° C. overnight under N₂ atmosphere. The mixture was concentrated and the residue was purified by column chromatography to afford the desired product (400 mg). ESI-MS (EI+, m/z): 344.10.

6. Synthesis of compound int. 4-6. A solution of 8-(4-phenoxyphenyl)-2-vinyl-5,8-dihydropyrido[2,3-d]pyrimidin-7(6H)-one (50 mg, 0.15 mmol) in MeOH (2 mL) was stirred at −78° C. under O₃ for 5 min. The solution was concentrated to afford title product (40 mg). ESI-MS (EI+, m/z): 345.36.

7. Synthesis of compound int. 4. To a solution of 7-oxo-8-(4-phenoxyphenyl)-5,6,7,8-tetrahydropyrido[2,3-d]pyrimidine-2-carbaldehyde (150 mg, 0.43 mmol) in MeOH (1 mL) was added BH₃DMS (2M, 2.15 mL) and the mixture was stirred at rt. The mixture was concentrated. The crude material was purified by column chromatography to get the desired product (60 mg). ESI-MS (EI+, m/z): 348.10. ¹H NMR (400 MHz, DMSO-d₆) δ 8.52 (s, 1H), 7.48-7.39 (m, 2H), 7.21 (dd, J=8.8, 2.4 Hz, 3H), 7.13-7.09 (m, 2H), 7.08-7.03 (m, 2H), 4.76 (dt, J=6.0, 3.2 Hz, 1H), 4.31 (d, J=6.0 Hz, 2H), 3.03 (t, J=7.6 Hz, 2H), 2.84 (dd, J=8.8, 6.4 Hz, 2H).

### Example 5. Preparation of Compound Int. 5

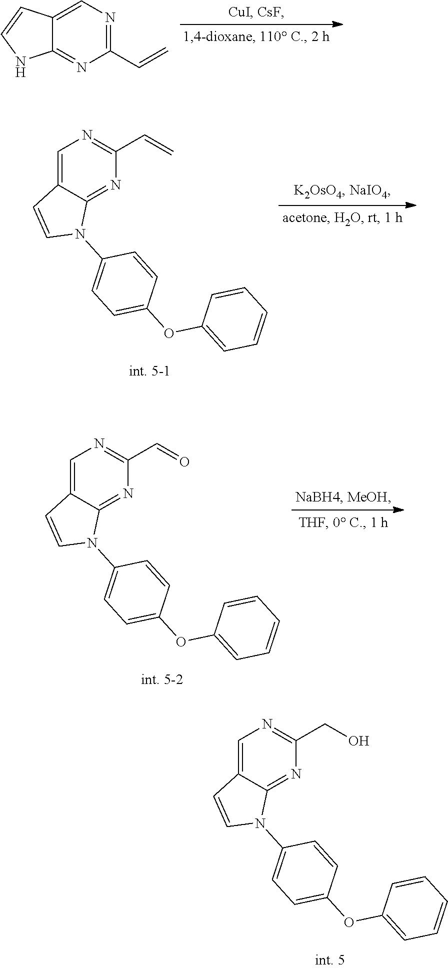 [SMILES: C=Cc1ncc2cc[nH]c2n1 ; C=Cc1ncc2ccn(-c3ccc(Oc4ccccc4)cc3)c2n1 ; O=Cc1ncc2ccn(-c3ccc(Oc4ccccc4)cc3)c2n1 ; OCc1ncc2ccn(-c3ccc(Oc4ccccc4)cc3)c2n1]

1. Synthesis of compound int. 5-1. A solution of 2-ethenyl-7H-pyrrolo[2,3-d]pyrimidine (1.0 g, 6.89 mmol), 1-iodo-4-phenoxybenzene (3.1 g, 10.3 mmol), CuI (656.1 mg, 3.45 mmol), (1R,2R)—N,N′-dimethyl-1,2-cyclohexanediamine (490 mg, 3.45 mmol) and CsF (3.2 g, 30.7 mmol) in 20 mL of 1,4-dioxane was stirred at 110° C. in an inert atmosphere for 2 h. The mixture was filtered to give organic phase. The crude was purified by silica gel column to give title compound (1.5 g). ¹H NMR (400 MHz, Chloroform-d) δ 9.01 (s, 1H), 7.73 (d, J=8.8 Hz, 2H), 7.47 (d, J=3.6 Hz, 1H), 7.39 (t, J=8.0 Hz, 2H), 7.20-7.13 (m, 3H), 7.12-7.07 (m, 2H), 6.96 (dd, J=17.2, 10.6 Hz, 1H), 6.68 (d, J=3.6 Hz, 1H), 6.60 (dd, J=17.2, 1.6 Hz, 1H), 5.64 (dd, J=10.4, 1.6 Hz, 1H).

2. Synthesis of compound int. 5-2. A solution of 7-(4-phenoxyphenyl)-2-vinyl-7H-pyrrolo[2,3-d]pyrimidine (400 mg, 1.28 mmol), K₂OsO₄·2H₂O (22.4 mg, 0.06 mmol), NaIO₄ (1.37 g, 6.38 mmol), 4-methylmorpholin-4-ium-4-olate (748 mg, 6.38 mmol) in acetone (8 mL) and H₂O (2.4 mL) was stirred at room temperature for 1 h. The mixture was added H₂O, extracted with EtOAc and the organic phase was concentrated to give crude. The crude was purified by silica gel column to give title compound (300 mg). ¹H NMR (400 MHz, DMSO-d₆) δ 10.02 (s, 1H), 9.37 (s, 1H), 8.29 (d, J=3.6 Hz, 1H), 7.91 7.85 (m, 2H), 7.45 (td, J=7.6, 2.0 Hz, 2H), 7.25-7.19 (m, 3H), 7.15-7.10 (m, 2H), 7.04 (s, 1H).

3. Synthesis of compound int. 5. To a stirred solution of 7-(4-phenoxyphenyl)-7H-pyrrolo[2,3-d]pyrimidine-2-carbaldehyde (300 mg, 0.95 mmol) in MeOH (4 mL) and THF (4 mL) was added sodium borohydride (72.0 mg, 1.9 mmol) at 0° C. and stirred for 1 h. The mixture was concentrated. The crude was purified by column chromatography to give title compound (200 mg). ¹H NMR (400 MHz, Chloroform-d) δ 9.03 (s, 1H), 7.64 (d, J=8.8 Hz, 2H), 7.48 (d, J=3.2 Hz, 1H), 7.41 (d, J=7.6 Hz, 2H), 7.19-7.08 (m, 6H), 6.74 (d, J=3.2 Hz, 1H), 4.91 (s, 2H).

### Example 6. Preparation of Compound Int. 6

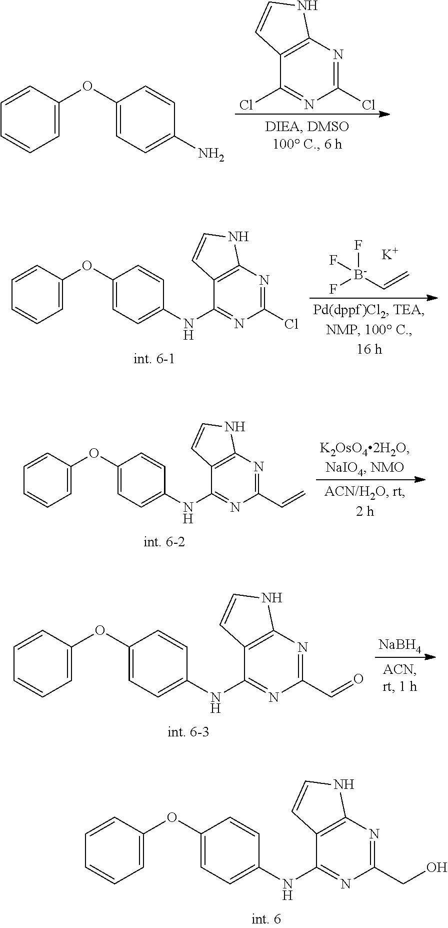

1. Synthesis of compound int. 6-1. A solution of 2,4-dichloro-7H-pyrrolo[2,3-d]pyrimidine (25.0 g, 133 mmol), 4-phenoxyaniline (24.6 g, 133 mmol) and DIEA (51.6 g, 399 mmol) in DMSO (100 mL) was stirred at 100° C. for 6 h. The solution was poured into water (500 mL), filtered. The residue was concentrated to give the crude product title compound (37.0 g).

2. Synthesis of compound int. 6-2. A mixture of 2-chloro-N-(4-phenoxyphenyl)-7H-pyrrolo[2,3-d]pyrimidin-4-amine (30.0 g, 89.1 mmol), Pd(dppf)₂Cl₂ (3.26 g, 4.45 mmol), TEA (36.1 g, 356 mmol) and potassium trifluoro(vinyl)borate (35.8 g, 267 mmol) in NMP (250 mL) was stirred at 100° C. under N₂ atmosphere for 16 h. The reaction mixture was filtered and concentrated. The residue was purified by column chromatography to give title compound (20 g).

3. Synthesis of compound int. 6-3. A solution of 2-ethenyl-N-(4-phenoxyphenyl)-7H-pyrrolo[2,3-d]pyrimidin-4-amine (5.0 g, 15.2 mmol) K₂OsO₄·2H₂O (267 mg, 0.76 mmol), 4-methylmorpholin-4-ium-4-olate (7.14 g, 60.9 mmol), NaIO₄ (16.3 g, 76.2 mmol) in H₂O (20 mL) and ACN (100 mL) was stirred at room temperature for 2 h. The reaction mixture was filtered and the filtrate was used directly in next step. ESI-MS (EI+, m/z): 331.

4. Synthesis of compound int. 6. To a solution of 4-[(4-phenoxyphenyl)amino]-7H-pyrrolo[2,3-d]pyrimidine-2-carbaldehyde (7.0 g, 21.2 mmol) in ACN (500 mL) was added sodium borohydride (2.40 g, 63.6 mmol) and stirred at room temperature for 1 h. The mixture was concentrated. The residue was purified by silica column chromatography to give title compound (8 g crude). ¹H NMR (400 MHz, DMSO-d₆) δ 11.65 (s, 1H), 9.31 (s, 1H), 8.07-7.83 (m, 2H), 7.49-7.29 (m, 2H), 7.18 (dd, J=3.6, 2.4 Hz, 1H), 7.10 (tt, J=7.2, 1.1 Hz, 1H), 7.04-6.97 (m, 4H), 6.72 (dd, J=3.6, 2.0 Hz, 1H), 4.48 (d, J=6.0 Hz, 2H).

### Example 7. Preparation of Compound Int. 7

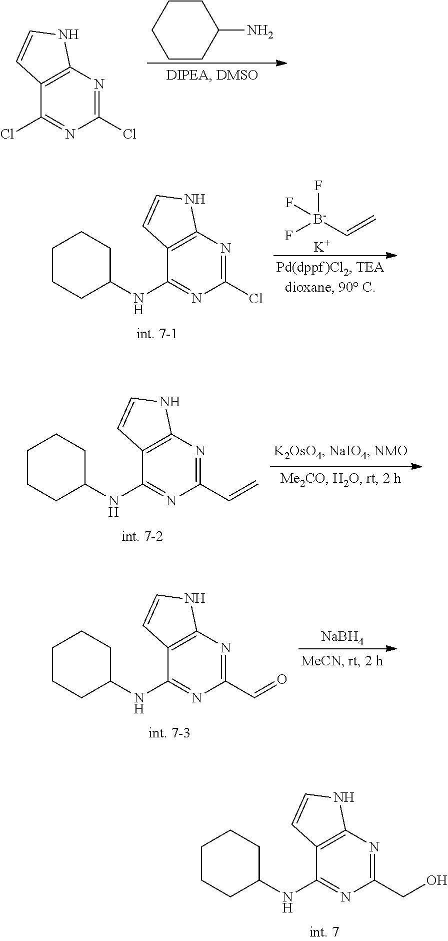

1. Synthesis of compound int. 7-1. To a solution of 2,4-dichloro-7H-pyrrolo[2,3-d]pyrimidine (10.0 g, 53.2 mmol) in DMSO (50 mL) was added cyclohexanamine (6.33 g, 63.8 mmol) and DIPEA (20.6 g, 160 mmol), the mixture was stirred at 25° C. for 6 h. The mixture was added water (500 mL) and stirred for 30 min, the precipate was filtered, dried under vacuum to give the desired product (10.5 g). ESI-MS (EI⁺, m/z): 251.25. ¹H NMR (400 MHz, Chloroform-d) δ 10.93 (s, 1H), 7.06 (d, J=3.6 Hz, 1H), 6.35 (d, J=3.6 Hz, 1H), 4.07 (s, 1H), 2.12 (dd, J=12.4, 4.0 Hz, 2H), 1.84-1.77 (m, 2H), 1.53-1.40 (m, 2H), 1.30 (qd, J=12.4, 6.0 Hz, 4H).

2. Synthesis of compound int. 7-2. To a solution of 2-chloro-N-cyclohexyl-7H-pyrrolo[2,3-d]pyrimidin-4-amine (2.1 g, 8.38 mmol) in 1,4-dioxane (50 mL) was added potassium vinyltrifluoroborate (5.61 g, 41.9 mmol), [1,1′-Bis(diphenylphosphino)ferrocene]dichloropalladium(II) (307 g, 0.42 mmol) and TEA (4.24 mg, 41.9 mmol). The mixture was stirred at 90° C. for 16 h under N₂ atmosphere, then concentrated. The residue was purified by column chromatography to afford title compound (750 mg). ESI-MS (EI⁺, m/z): 243.30. ¹H NMR (400 MHz, Chloroform-d) δ 11.49 (s, 1H), 7.10 (d, J=3.6 Hz, 1H), 6.82 (dd, J=17.2, 10.5 Hz, 1H), 6.53 (dd, J=17.2, 2.0 Hz, 1H), 6.39 (d, J=3.6 Hz, 1H), 5.66-5.58 (m, 1H), 4.25-4.15 (m, 2H), 2.20 (dd, J=12.8, 4.0 Hz, 2H), 1.85 (dt, J=13, 3.6 Hz, 2H), 1.72 (ddd, J=10.4, 7.6, 3.8 Hz, 1H), 1.57-1.43 (m, 2H), 1.41-1.28 (m, 5H).

3. Synthesis of compound int. 7-3. To a solution of N-cyclohexyl-2-ethenyl-7H-pyrrolo[2,3-d]pyrimidin-4-amine (2.0 g, 8.25 mmol) in acetone (50 mL) and H₂O (10 mL) was added K₂OsO₄·2H₂O (145 mg, 0.41 mmol), NaIO₄ (4.41 g, 20.6 mmol) and 4-methylmorpholin-4-ium-4-olate (2.42 mg, 20.6 mmol) at 0° C. The mixture was stirred at room temperature for 2 h. The mixture was filtered and concentrated under reduced pressure to afford title compound. ESI-MS (EI⁺, m/z): 245.05.

4. Synthesis of compound int. 7. To a solution of 4-(cyclohexylamino)-7H-pyrrolo[2,3-d]pyrimidine-2-carbaldehyde (2.02 mg, 8.25 mmol) in MeCN (50 mL) was added sodium boranuide (936 mg, 24.7 mmol). The mixture was stirred at room temperature for 2 h, then concentrated. The residue was purified by column chromatography to afford title compound (180 mg). ESI-MS (EI⁺, m/z): 247.30. ¹H NMR (400 MHz, Chloroform-d) δ 10.11 (s, 1H), 7.02 (d, J=3.6 Hz, 1H), 6.35 (d, J=3.6 Hz, 1H), 5.04 (s, 1H), 4.69 (s, 2H), 4.16-4.07 (m, 1H), 2.17-2.10 (m, 2H), 1.84-1.78 (m, 2H), 1.69 (td, J=7.6, 3.8 Hz, 2H), 1.52-1.40 (m, 3H), 1.35-1.27 (m, 4H).

### Example 8. Preparation of Compound Int. 8

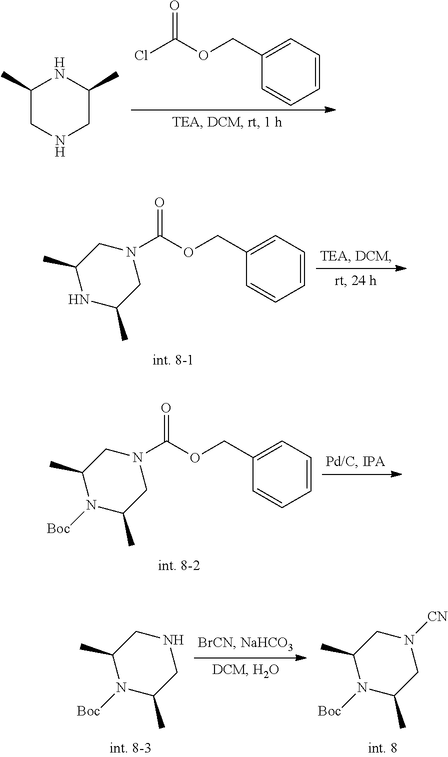

1. Synthesis of compound int. 8-1. To a solution of (2R,6S)-2,6-dimethylpiperazine (1.0 g, 8.76 mmol) in DCM (8 mL) was added CbzCl (1.49 g, 8.76 mmol), TEA (886 mg, 8.76 mmol) at 0° C. and stirred at room temperature for 1 h. The reaction solution was concentrated in vacuo, purified by silica gel column chromatography to get the desired product (2.1 g). ESI-MS (EI⁺, m/z): 249.01. ¹H NMR (400 MHz, DMSO-d₆) δ 7.4-7.29 (m, 5H), 5.07 (s, 2H), 3.83 (d, J=12.4 Hz, 2H), 2.59 (dqd, J=9.4, 6.2, 2.9 Hz, 2H), 2.29 (d, J=29.5 Hz, 2H), 0.94 (d, J=6.2 Hz, 6H).

2. Synthesis of compound int. 8-2. A solution of benzyl (3R,5S)-3,5-dimethylpiperazine-1-carboxylate (2.1 g, 8.46 mmol), TEA (1.28 g, 12.7 mmol), (Boc)₂O (2.03 g, 9.3 mmol) in DCM (50 mL) was stirred at room temperature for 24 h. The mixture was concentrated in vacuo, the residue was purified by column chromatography to get title compound (515 mg). ESI-MS (EI⁺, m/z): 349.11. ¹H NMR (400 MHz, DMSO-d₆) δ 7.43-7.27 (m, 5H), 5.12 (d, J=6.8 Hz, 2H), 4.03 (d, J=4.9 Hz, 2H), 3.83 (d, J=13.2 Hz, 2H), 3.02 (s, 2H), 1.40 (s, 9H), 1.07 (d, J=6.8 Hz, 6H).

3. Synthesis of compound int. 8-3. A solution of 4-benzyl 1-tert-butyl (2R,6S)-2,6-dimethylpiperazine-1,4-dicarboxylate (500 mg, 1.43 mmol), Pd/C (50 mg) in IPA (10 mL) was stirred at 25° C. for 24 h under H₂ atmosphere. The mixture was filtered, concentrated in vacuo to get title compound (226 mg). ¹H NMR (400 MHz, DMSO-d₆): δ 3.82 (qdd, J=6.8, 4.6, 1.3 Hz, 2H), 2.67 (dt, J=12.1, 1.2 Hz, 2H), 2.60 (dd, J=12.2, 4.4 Hz, 2H), 1.39 (s, 9H), 1.15 (d, J=6.8 Hz, 6H).

4. Synthesis of compound int. 8. To a solution of tert-butyl (2R,6S)-2,6-dimethylpiperazine-1-carboxylate (266 mg, 1.24 mmol), NaHCO₃ (312.52 mg, 3.72 mmol) in H₂O (4 mL) and DCM (2 mL) was added BrCN (164 mg, 1.55 mmol) at 0° C. and stirred at 25° C. for 16 h. The solution was concentrated in vacuo, purified by column chromatography to get title compound (260 mg). ¹H NMR (400 MHz, DMSO-d₆) δ 4.07-4.01 (m, 2H), 3.26 (d, J=12.8 Hz, 2H), 3.22-3.16 (m, 2H), 1.41 (s, 9H), 1.24 (d, J=6.8 Hz, 6H).

### Example 9. Preparation of Compound Int. 9

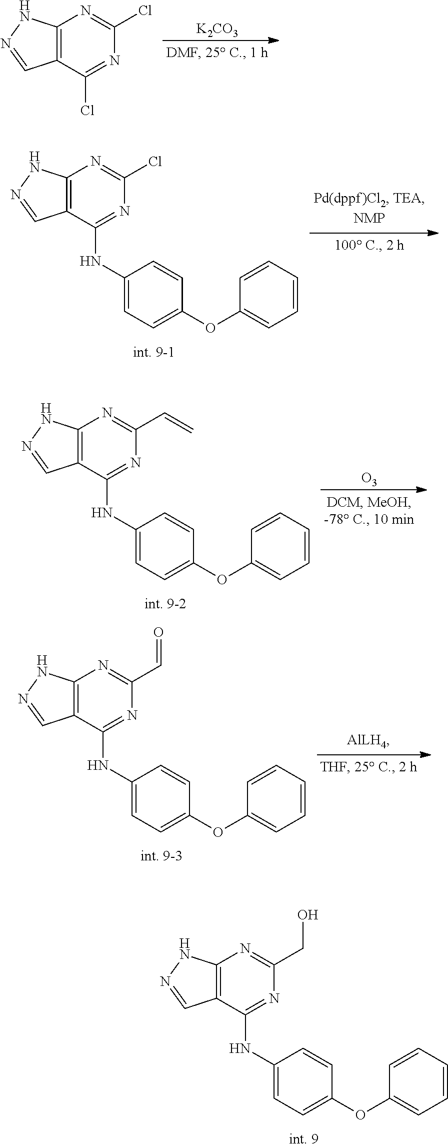

1. Synthesis of compound int. 9-1. A solution of 4,6-dichloro-1H-pyrazolo[3,4-d]pyrimidine (10 g, 52.9 mmol) and 4-phenoxyaniline (9.31 g, 50.3 mmol) in DMF (30 mL) was added K₂CO₃ (14.6 g, 106 mmol) at 25° C. and stirred for 1 h. The reaction mixture was purified by column chromatography to get title compound (4.0 g). ESI-MS (EI⁺, m/z): 338.25. ¹H NMR (400 MHz, DMSO-d₆) δ 13.75 (s, 1H), 10.40 (s, 1H), 7.74 (s, 2H), 7.44-7.37 (m, 2H), 7.17-7.02 (m, 5H).

2. Synthesis of compound int. 9-2. A solution of 6-chloro-N-(4-phenoxyphenyl)-1H-pyrazolo[3,4-d]pyrimidin-4-amine (2.0 g, 5.92 mmol) in NMP (5 mL) was added potassium trifluoro(vinyl)borate (3.96 g, 29.6 mmol), Pd(dppf)Cl2 (434 mg, 0.59 mmol), TEA (3.0 g, 29.6 mmol) and stirred at 100° C. for 2 h. The mixture was diluted with EtOAc and washed with a solution of aqueous NaHCO₃. The organic layer was dried over sodium sulfate and concentrated. The residue was purified by column chromatography to get title compound (800 mg). ESI-MS (EI⁺, m/z): 330.25. ¹H NMR (400 MHz, DMSO-d₆) δ 13.55 (s, 1H), 9.97 (s, 1H), 8.27-8.17 (m, 1H), 7.92 (d, J=8.5 Hz, 2H), 7.43-7.36 (m, 2H), 7.16-7.07 (m, 3H), 7.05-7.00 (m, 2H), 6.70 (dd, J=17.2, 10.3 Hz, 1H), 6.48 (dd, J=17.2, 2.2 Hz, 1H), 5.67 (dd, J=10.4, 2.2 Hz, 1H).

3. Synthesis of compound int. 9-3. To a solution of 6-ethenyl-N-(4-phenoxyphenyl)-1H-pyrazolo[3,4-d]pyrimidin-4-amine (300 mg, 0.9 mmol) in MeOH (15 mL) and DCM (10 mL) was stirred at −78° C. for 10 min under O₃ atmosphere. The solvent was evaporated to dryness to afford title compound.

4. Synthesis of compound int. 9. To a solution of 4-[(4-phenoxyphenyl)amino]-1H-pyrazolo[3,4-d]pyrimidine-6-carbaldehyde (300 mg, 0.91 mmol) and sodium methanolate (48.9 mg, 0.91 mmol) in THF (5 mL) was added LiAlD₄ (103 mg, 2.72 mmol) and the mixture was stirred at 25° C. for 2 h under a N₂ atmosphere. The mixture was quenched with EtOH, filtered and the filtrate was concentrated. The residue was purified by column chromatography to afford title compound (90 mg). ESI-MS (EI⁺, m/z): 334.10.

### Example 10. Preparation of Compound Int. 10

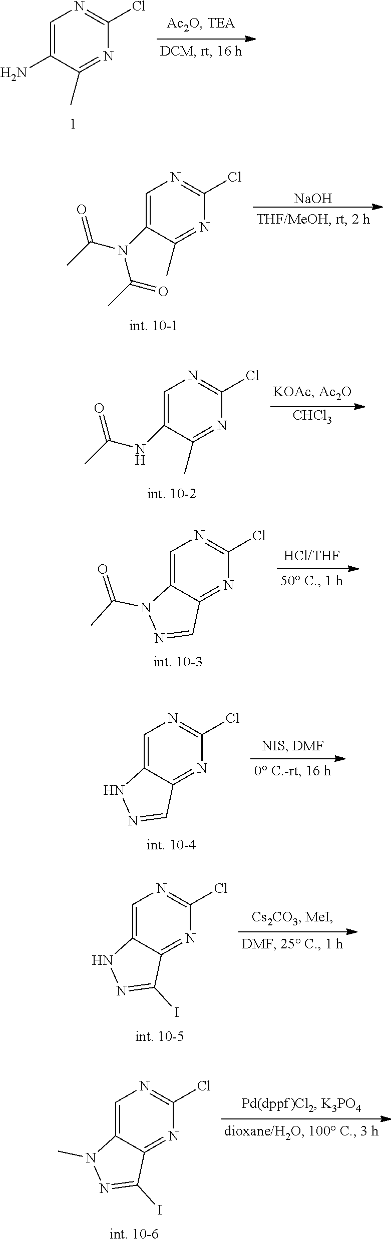

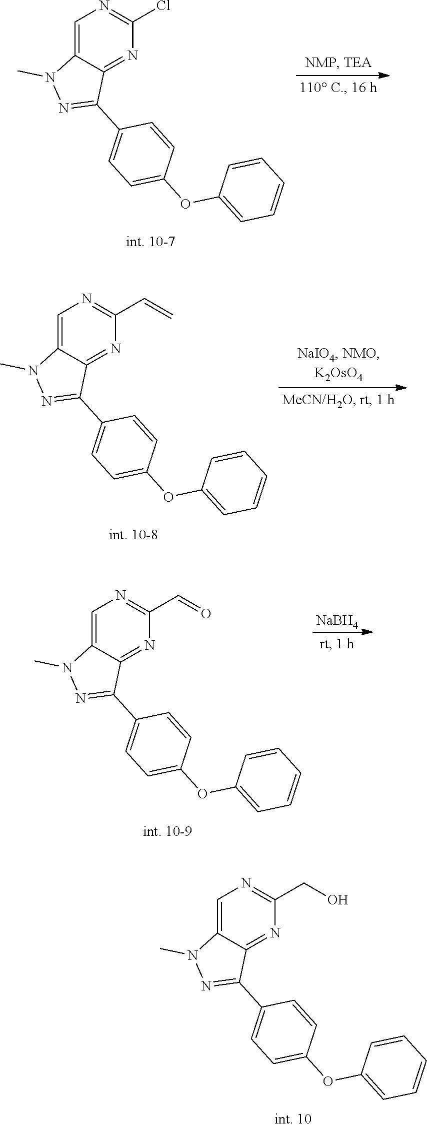 [SMILES: Cn1nc(-c2ccc(Oc3ccccc3)cc2)c2nc(Cl)ncc21 ; C=Cc1ncc2c(n1)c(-c1ccc(Oc3ccccc3)cc1)nn2C ; Cn1nc(-c2ccc(Oc3ccccc3)cc2)c2nc(C=O)ncc21 ; "NaBH4" ; Cn1nc(-c2ccc(Oc3ccccc3)cc2)c2nc(CO)ncc21]

1. Synthesis of compound int. 10-1. A solution of 2-chloro-4-methylpyrimidin-5-amine (4.0 g, 6.97 mmol), Ac₂O (747 mg, 7.32 mmol), TEA (1763 mg, 17.4 mmol) in DCM (40 mL) was stirred at room temperature for 16 h. The solvent was removed and the residue was purified by silica gel column to afford title compound (3.60 g). ESI-MS (EI⁺, m/z): 228.10. ¹H NMR (400 MHz, DMSO-d₆) δ 8.72 (s, 1H), 2.34 (s, 3H), 2.26 (s, 6H).

2. Synthesis of compound int. 10-2. To a solution of N-acetyl-N-(2-chloro-4-methylpyrimidin-5-yl)acetamide (3.60 g, 15.8 mmol) in MeOH (1 mL) and THF (20 mL) was added NaOH aqueous solution (949 mg, 23.7 mmol, 1 M). The reaction solution was stirred at room temperature for 1 h. The reaction was acidified with HCl (12 M) to adjust pH=6-7, then extracted with EtOAc (2×50 mL). The organic layers were dried over Na₂SO₄, filtered and concentrated to afford title compound (3.0 g).

3. Synthesis of compound int. 10-3. A mixture of N-(2-chloro-4-methylpyrimidin-5-yl)acetamide (5.0 g, 26.9 mmol), KOAc (1.90 mg, 19.4 mmol), Ac₂O (9.90 g, 97.0 mmol), amyl nitrite (7.89 g, 67.4 mmol) in CHCl₃ (50 mL) was stirred at 60° C. for 16 h. The reaction was concentrated to dryness. The crude was purified by silica gel column to afford title compound (3.2 g). ESI-MS (EI⁺, m/z): 197.00.

¹H NMR (400 MHz, DMSO-d₆) δ 9.66 (d, J=0.8 Hz, 1H), 8.84 (d, J=0.7 Hz, 1H), 2.76 (s, 3H).

4, Synthesis of compound int. 10-4. To a solution of 1-(5-chloro-1H-pyrazolo[4,3-d]pyrimidin-1-yl)ethan-1-one (3.20 g, 16.3 mmol) in THF (30 mL) was added HCl (8%, 30 mL), the solution was stirred at 50° C. for 1 h. The reaction solution was extracted with EtOAc (2×50 mL). The organic layers were dried over Na₂SO₄, filtered and concentrated to afford title compound (2.50 g). ¹H NMR (400 MHz, DMSO-d₆) δ 14.16 (s, 1H), 9.33 (s, 1H), 8.44 (s, 1H).

5. Synthesis of compound int. 10-5. To a solution of 5-chloro-1H-pyrazolo[4,3-d]pyrimidine (2.50 g, 16.2 mmol) in DMF (20 mL) was added NIS (6.55 g, 29.1 mmol) at 0° C., then the reaction was warmed to room temperature and stirred for 16 h. The reaction solution was concentrated. The crude was purified by silica gel column to afford title compound (3.34 g). ESI-MS (EI⁺, m/z): 280.90.

6. Synthesis of compound int. 10-6. A mixture of 5-chloro-3-iodo-1H-pyrazolo[4,3-d]pyrimidine (3.34 g, 11.9 mmol), iodomethane (2.03 g, 14.3 mmol) and Cs₂CO₃ (4.66 g, 14.3 mmol) in DMF (8 mL) was stirred at 25° C. for 1 h. The reaction mixture was filtered. The residue was diluted with EtOAc (50 mL) and washed with H₂O (3×30 mL). The organic layer was dried, filtered and concentrated to afford title compound (3.10 g).

7. Synthesis of compound int. 10-7. A mixture of 5-chloro-3-iodo-1-methyl-1H-pyrazolo[4,3-d]pyrimidine (3.10 g, 10.5 mmol), 4-phenoxyphenylboronic acid (2.03 g, 9.47 mmol), K₃PO₄ (2.87 g, 21.1 mmol), Pd(dppf)Cl₂ (771 mg, 1.05 mmol) in dioxane (30 mL) and H₂O (6 mL) was stirred under N₂ atmosphere at 100° C. for 3 h. The reaction was filtered and concentrated to dryness. The crude was purified by silica gel column and Prep-HPLC to afford title compound (1.78 g). ESI-MS (EI⁺, m/z): 337.05. ¹H NMR (400 MHz, DMSO-d₆) δ 9.48 (s, 1H), 8.35-8.25 (m, 2H), 7.50-7.38 (m, 2H), 7.24-7.16 (m, 3H), 7.15-7.08 (m, 2H), 4.24 (s, 3H).

8. Synthesis of compound int. 10-8. A mixture of 5-chloro-1-methyl-3-(4-phenoxyphenyl)-1H-pyrazolo[4,3-d]pyrimidine (1.78 g, 5.29 mmol), potassium trifluoro(vinyl)borate (2.8 g, 21.2 mmol), TEA (1.61 g, 15.9 mmol), Pd(dppf)Cl₂ (385 g, 0.53 mmol) in NMP (20 mL) was stirred under N₂ at 110° C. for 16 h. The reaction was diluted with EtOAc (50 mL), filtered and washed with LiCl solution (3×100 mL). The organic layer was concentrated. The crude was purified by silica gel column to afford title compound (1.20 g). ESI-MS (EI⁺, m/z): 329.15. ¹H NMR (400 MHz, DMSO-d₆) δ 9.46 (s, 1H), 8.48-8.40 (m, 2H), 7.47-7.40 (m, 2H), 7.21-7.08 (m, 5H), 6.99 (dd, J=17.3, 10.5 Hz, 1H), 6.59 (dd, J=17.3, 2.0 Hz, 1H), 5.73 (dd, J=10.6, 2.0 Hz, 1H), 4.22 (s, 3H).

9. Synthesis of compound int. 10-9. To a solution of 1-methyl-3-(4-phenoxyphenyl)-5-vinyl-1H-pyrazolo[4,3-d]pyrimidine (500 mg, 1.52 mmol) in MeCN (30 mL) and H₂O (10 mL) was added K₂OsO₄·2H₂O (53.4 mg, 0.15 mmol), NaIO₄ (1.63 g, 7.61 mmol), 4-methylmorpholin-4-ium-4-olate (892 mg, 7.61 mmol). The reaction solution was stirred at room temperature for 1 h. The reaction mixture was filtered and the residue was used to next step directly. ESI-MS (EI⁺, m/z): 331.10.

10. Synthesis of compound int. 10. To a solution of 1-methyl-3-(4-phenoxyphenyl)-1H-pyrazolo[4,3-d]pyrimidine-5-carbaldehyde (500 mg, 1.51 mmol) in MeCN (30 mL) and H₂O (10 mL) was added NaBH₄ (142 mg, 2.27 mmol) at 0° C. and then stirred at room temperature for 1 h. The reaction mixture was purified by silica gel column to afford title compound (280 mg). ESI-MS (EI⁺, m/z): 331.10. ¹H NMR (400 MHz, DMSO-d₆) δ 9.45 (s, 1H), 8.51-8.43 (m, 2H), 7.48-7.39 (m, 2H), 7.22-7.07 (m, 5H), 5.22 (t, J=6.2 Hz, 1H), 4.79 (d, J=6.1 Hz, 2H), 4.23 (s, 3H).

### Example 11. Preparation of Compound Int. 11

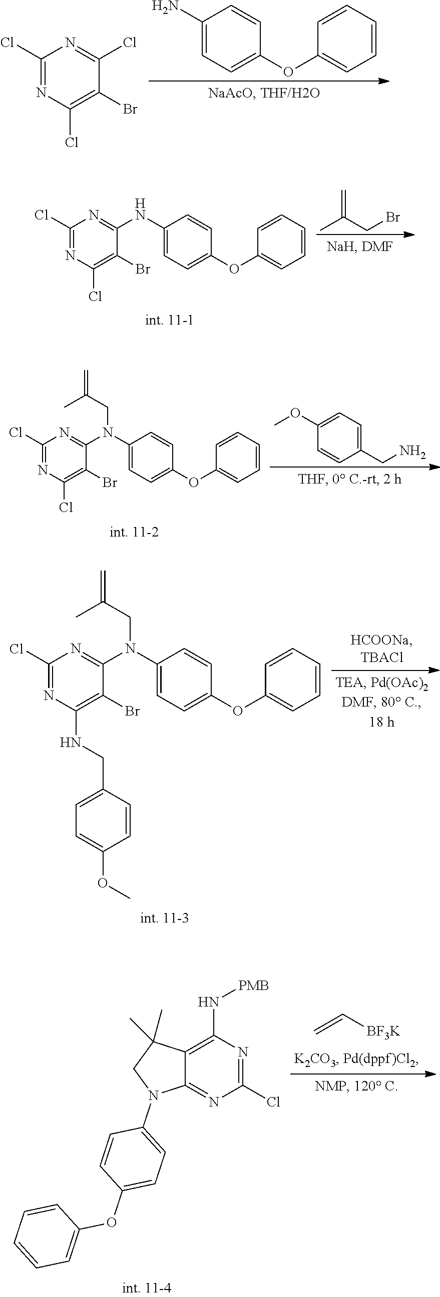

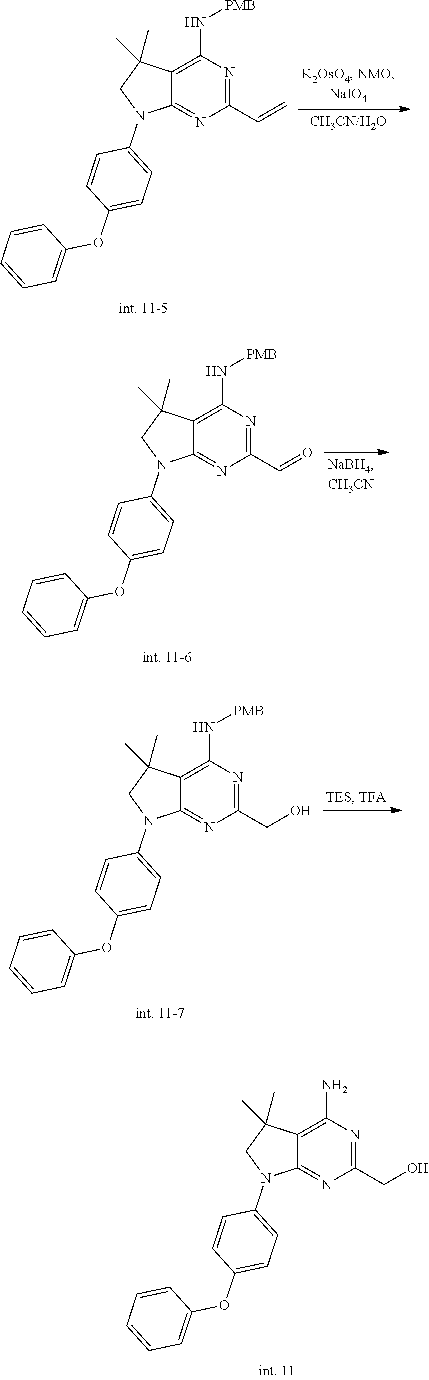

1. Synthesis of compound int. 11-1. To a solution of 5-bromo-2,4,6-trichloropyrimidine (3.00 g, 11.4 mmol) in THF (20 mL)/water (10 mL) was added 4-phenoxyaniline (2.14 mg, 11.5 mmol) and sodium acetate (4.67 g, 34.3 mmol) at 25° C. under N₂ atmosphere, then the reation was stirred at 25° C. for 12 h. The mixture was purified by flash column chromatography to afford title compound (4.30 g, purity: 85%). ESI-MS (EI⁺, m/z): 412.00. ¹H NMR (400 MHz, DMSO-d₆) δ 9.55 (s, 1H), 7.54-7.44 (m, 2H), 7.44-7.34 (m, 2H), 7.16 (t, J=7.2 Hz, 1H), 7.08-6.98 (m, 4H).

2. Synthesis of compound int. 11-2. To a stirred solution of compound 5-bromo-2,6-dichloro-N—(4-phenoxyphenyl)pyrimidin-4-amine (4.30 g, 0.46 mmol) in N,N-dimethylformamide (20 mL) was added NaH (586 mg, 14.6 mmol, 60%) at 25° C. under N₂ atmosphere. After 1 h, 3-bromo-2-methylprop-1-ene (2.26 g, 16.7 mmol) was added, then the reaction was stirred at 25° C. for 12 h. The reaction mixture was purified by flash column chromatography to afford title compound (3.26 g, purity 85%). ESI-MS (EI⁺, m/z): 466.05. ¹H NMR (400 MHz, DMSO-d₆) δ 7.43-7.37 (m, 2H), 7.31-7.25 (m, 2H), 7.19-7.10 (m, 1H), 7.01 (ddt, J=11.6, 9.6, 2.0 Hz, 4H), 4.91-4.80 (m, 2H), 4.57 (s, 2H), 1.73 (s, 3H).

3. Synthesis of compound int. 11-3. To a solution of Compound 5-bromo-2,6-dichloro-N-(2-methylallyl)-N-(4-phenoxyphenyl)pyrimidin-4-amine (3.07 g, 5.61 mmol, purity 85%) in THF (30 mL) was added 4-methoxybenzylamine (2.85 mg, 20.8 mmol) at 0° C. under N₂ atmosphere, then the reaction was stirred at room temperature for 1.5 h before evaporated. The residue was purified by flash column chromatography to afford title compound. ESI-MS (EI⁺, m/z): 567.15. ¹H NMR (400 MHz, DMSO-d₆) δ 7.77 (t, J=6.0 Hz, 1H), 7.37 (dd, J=8.4, 7.2 Hz, 2H), 7.28-7.21 (m, 2H), 7.11 (s, 1H), 7.03 (d, J=9.0 Hz, 2H), 7.00-6.92 (m, 4H), 6.90-6.84 (m, 2H), 4.91-4.78 (m, 2H), 4.45 (d, J=6.4 Hz, 4H), 3.71 (s, 3H), 1.72 (s, 3H).

4. Synthesis of compound int. 11-4. A mixture of compound 5-bromo-2-chloro-N4-(4-methoxybenzyl)-N6-(2-methylallyl)-N6-(4-phenoxyphenyl)pyrimidine-4,6-diamine (2.46 g, 4.35 mmol), tetrabutylammonium chloride (1.21 g, 4.35 mmol), TEA (5.52 g, 54.6 mmol), diacetoxypalladium (195 mg, 0.87 mmol) in N,N-dimethylformamide (20 mL) was stirred at 80° C. for 18 h under N₂ atmosphere. The mixture was evaporated and purified by flash column chromatography to afford title compound (2.00 g, purity 85%). ESI-MS (EI⁺, m/z): 487.20. ¹H NMR (400 MHz, DMSO-d₆) δ 7.64 (d, J=9.1 Hz, 2H), 7.36 (dd, J=8.6, 7.4 Hz, 2H), 7.26-7.18 (m, 2H), 7.12-7.01 (m, 3H), 7.01-6.93 (m, 3H), 6.88 (d, J=8.7 Hz, 2H), 4.51 (d, J=6.1 Hz, 2H), 3.75 (s, 2H), 3.72 (s, 3H), 1.42 (s, 6H).

5. Synthesis of compound int. 11-5. A mixture of compound 2-chloro-N-(4-methoxybenzyl)-5,5-dimethyl-7-(4-phenoxyphenyl)-6,7-dihydro-5H-pyrrolo[2,3-d]pyrimidin-4-amine (1.44 g, 2.66 mmol), potassium vinyltrifluoroborate (1.78 mg, 13.3 mmol), Pd(dppf)Cl₂ (194 mg, 0.27 mmol), K₂CO₃ (1.10 g, 7.98 mmol) in NMP (8 mL) was stirred at 120° C. for 12 h under N₂ atmosphere. The solvent was evaporated to dryness and purified by flash column chromatography to afford title compound (728 mg, purity: 70%). ESI-MS (EI+, m/z): 479.30. ESI-MS (EI−, m/z): 477.25. ¹H NMR (400 MHz, DMSO-d₆) δ 7.92-7.77 (m, 2H), 7.39-7.32 (m, 2H), 7.27 (d, J=8.5 Hz, 2H), 7.12-7.00 (m, 3H), 6.98-6.91 (m, 2H), 6.89-6.82 (m, 2H), 6.64 (t, J=6.2 Hz, 1H), 6.49 (dd, J=17.2, 10.3 Hz, 1H), 6.31 (dd, J=17.3, 2.5 Hz, 1H), 5.49 (dd, J=10.3, 2.5 Hz, 1H), 4.59 (d, J=6.1 Hz, 2H), 3.70 (d, J=3.0 Hz, 5H), 1.43 (s, 6H).

6. Synthesis of compound int. 11-6. A mixture of compound N-(4-methoxybenzyl)-5,5-dimethyl-7-(4-phenoxyphenyl)-2-vinyl-6,7-dihydro-5H-pyrrolo[2,3-d]pyrimidin-4-amine (728 mg, 1.52 mmol), K₂OSO₄·2H₂O (53.3 mg, 0.15 mmol), 4-methylmorpholin-4-ium-4-olate (890 mg, 7.6 mmol), Na₁O₄ (1.63 g, 7.6 mmol) in acetonitrile (50 mL)/Water (5 mL) was stirred at 25° C. for 2 h under N₂ atmosphere. The reaction mixture was filtered and the filtrate was concentrated to give the title compound (731 g). ESI-MS (EI+, m/z): 481.25.

7. Synthesis of compound int. 11-7. A mixture of compound 4-((4-methoxybenzyl)amino)-5,5-dimethyl-7-(4-phenoxyphenyl)-6,7-dihydro-5H-pyrrolo[2,3-d]pyrimidine-2-carbaldehyde (731 g, 1.52 mmol) in acetonitrile (15 mL) was added sodium borohydride (115.02 mg, 3.04 mmol) at rt, then the reaction was stirred at room temperature for 0.4 h under N₂ atmosphere. The reaction mixture was filtered. The filtrate was concentrated and purified by column chromatography to give title compound (717 mg, purity: 85%). ESI-MS (EI⁺, m/z): 483.30. ¹H NMR (400 MHz, DMSO-d₆) δ 7.83-7.75 (m, 2H), 7.41-7.31 (m, 2H), 7.26 (d, J=8.5 Hz, 2H), 7.14-7.04 (m, 1H), 7.04-6.91 (m, 4H), 6.91-6.83 (m, 2H), 6.65 (t, J=6.2 Hz, 1H), 4.60 (d, J=6.1 Hz, 2H), 4.50 (t, J=5.7 Hz, 1H), 4.25 (d, J=5.7 Hz, 2H), 3.70 (d, J=3.2 Hz, 5H), 1.42 (s, 6H).

8. Synthesis of compound int. 11. A mixture of compound (4-((4-methoxybenzyl)amino)-5,5-dimethyl-7-(4-phenoxyphenyl)-6,7-dihydro-5H-pyrrolo[2,3-d]pyrimidin-2-yl)methanol (200 mg, 0.41 mmol) in TFA (2 mL) was added TES (0.1 mL) at rt, then the reaction was stirred at 25° C. for 1 h under N₂ atmosphere. The solvent was evaporated and purified by column chromatography to give title compound (206 mg, purity: 85%). ESI-MS (EI+, m/z): 363.20. ¹H NMR (400 MHz, DMSO-d₆) δ 7.74-7.65 (m, 2H), 7.44-7.34 (m, 2H), 7.33-7.16 (m, 3H), 7.13 (t, J=7.4 Hz, 1H), 7.05 (d, J=9.0 Hz, 2H), 6.99 (d, J=7.8 Hz, 2H), 4.50 (s, 2H), 3.92 (s, 2H), 1.43 (s, 6H).

### Example 12. Preparation of Compound Int. 12

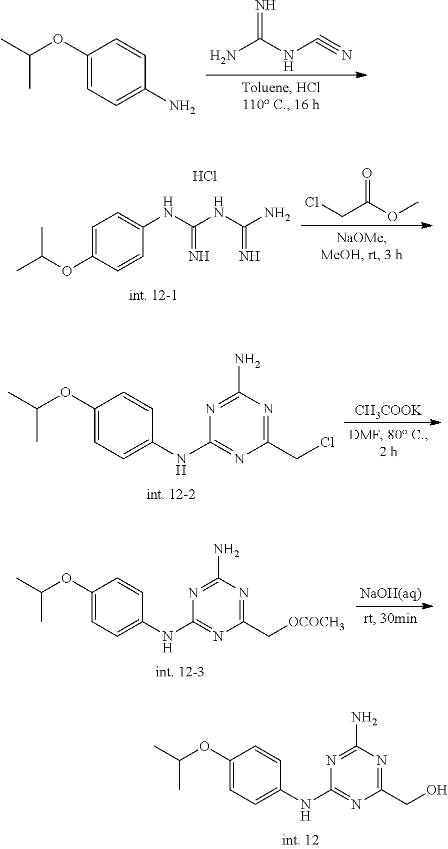

1. Synthesis of compound int. 12-1. A solution of 4-isopropoxyaniline (1.98 g, 13.1 mmol) and dicyandiamide (1.0 g, 11.9 mmol) in toluene (30 mL) was stirred at 25° C. for 15 min before 5% HCl (7 mL) was added. The solution was then heated to 110° C. and stirred for 16 h. The reaction was filtered. The filter cake was collected and concentrated to afford title compound (2.0 g). ¹H NMR (400 MHz, DMSO-d₆) δ 9.42 (s, 1H), 7.23-7.18 (m, 2H), 7.14 (s, 4H), 6.97 (s, 2H), 6.89-6.82 (m, 2H), 4.54 (h, J=6.0 Hz, 1H), 1.24 (d, J=6.0 Hz, 6H).

2. Synthesis of compound int. 12-2. A solution of int. 12-1 (100 mg, 0.42 mmol), methyl 2-chloroacetate (46 mg, 0.42 mmol) and 30% NaOMe/MeOH solution (115 mg, 0.64 mmol) in MeOH (3 mL) was stirred at room temperature for 3 h, then was purified by Prep-HPLC to afford title compound (18 mg). ESI-MS (EI⁺, m/z): 294.15.

3. Synthesis of compound int. 12-3. A mixture of 6-(chloromethyl)-N2-(4-isopropoxyphenyl)-1,3,5-triazine-2,4-diamine (1.00 g, 3.4 mmol) and KOAc (668 mg, 6.8 mmol) in DMF (10 mL) was stirred at 80° C. for 2 h. The reaction was diluted with EtOAc (100 mL) and washed with brine (3×60 mL). The organic layer was dried, filtered and concentrated to afford title compound (1.00 g). ESI-MS (EI⁺, m/z):318.20.

4. Synthesis of compound int. 12. To a solution of (4-amino-6-((4-isopropoxyphenyl)amino)-1,3,5-triazin-2-yl)methyl acetate (500 mg, 1.58 mmol) in MeOH (5 mL) was added NaOH aqueous solution (3.2 mL, 3.2 mmol). The reaction was then stirred at room temperature for 30 min. The solvent was removed, the residue was neutralized and extracted with EtOAc (2×50 mL). The organic layer was dried over Na₂SO₄, filtered and concentrated to afford title compound (400 mg). ESI-MS (EI⁺, m/z): 276.15.

### Example 13. Preparation of Compound Int. 13

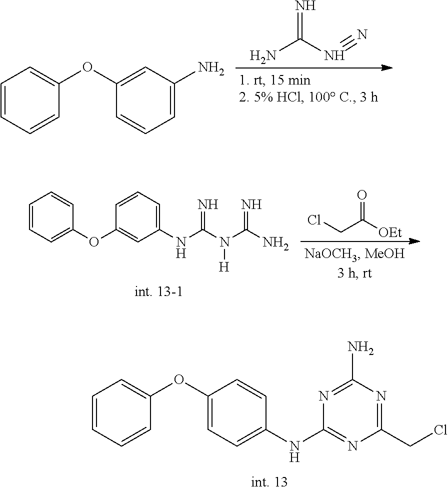

1. Synthesis of compound int. 13-1. A solution of 3-phenoxyaniline (4.0 g, 21.6 mmol), dicyandiamide (2.00 g, 23.8 mmol) in 40 mL of toluene was stirred at room temperature for 15 minutes. After that, a mixture of 50% HCl aqueous solution (14 mL, v/v) was added dropwise. The mixture was stirred at 100° C. for 3 h. The reaction was filtered through MeCN and DCM to afford the title compound (3.0 g).

2. Synthesis of compound int. 13. A solution of int. 13-1 (3.0 g, 11.1 mmol), ethyl 2-chloroacetate (1.36 g, 11.1 mmol) and NaOCH₃ (4.0 g, 22 mmol) in MeOH (600 mL) was stirred at room temperature for 3 h. After that, 5 mL of HCl aqueous solution (35%, v/v) was added dropwise and the mixture was stirred at room temperature for 2 h. 30 mL of water was added to the mixture and filtered to afford title compound (1.44 g). ¹H NMR (400 MHz, DMSO-d₆) δ 9.79 (s, 1H), 7.78 (d, J=9.0 Hz, 2H), 7.36 (td, J=7.5, 1.9 Hz, 4H), 7.09 (t, J=7.4 Hz, 1H), 7.00-6.93 (m, 4H), 4.36 (s, 2H).

### Example 14. Preparation of Compound Int. 14

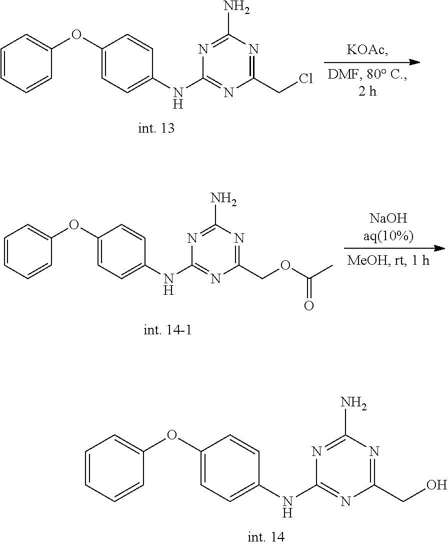 [SMILES: Nc1nc(CCl)nc(Nc2ccc(Oc3ccccc3)cc2)n1 ; CC(=O)OCc1nc(N)nc(Nc2ccc(Oc3ccccc3)cc2)n1 ; Nc1nc(CO)nc(Nc2ccc(Oc3ccccc3)cc2)n1]

1. Synthesis of compound int. 14-1. A solution of int. 13 (1.44 g, 4.40 mmol), KOAc (2.16 g, 22.0 mmol) in DMF (20 mL) was stirred at 80° C. for 2 h. The reaction was purified by column chromatography to afford title compound (1.50 g). ESI-MS (EI⁺, m/z): 352.15.

2. Synthesis of compound int. 14. A solution of (4-amino-6-((4-phenoxyphenyl)amino)-1,3,5-triazin-2-yl)methyl acetate (1.50 g, 4.27 mmol) and 10% aqueous solution NaOH (20 mL) in MeOH (20 mL) was stirred at room temperature for 1 h. The reaction was extracted with EtOAc and H₂O. The residue was purified by column chromatography to afford the title compound (1.05 g). ESI-MS (EI⁺, m/z): 310.15.

### Example 15. Preparation of Compound Int. 15

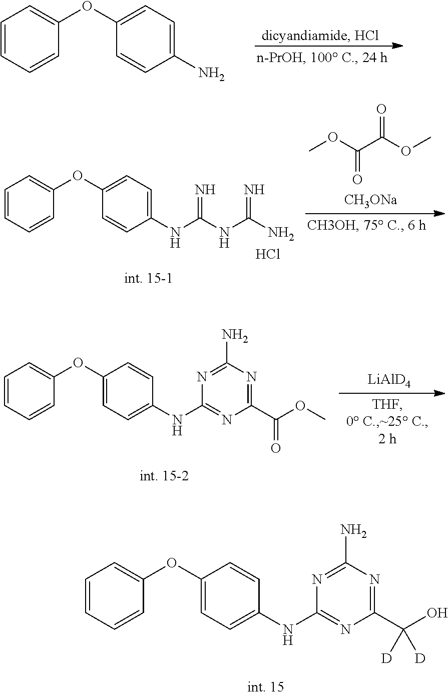

1. Synthesis of compound int. 15-1. To a solution of 4-phenoxyaniline (1.85 g, 9.99 mmol) in n-PrOH (20 mL) were added dicyandiamide (840 mg, 9.99 mmol) and concentrated hydrogen chloride (0.95 mL, 10.99 mmol). The mixture was heated up to 100° C. for 24 h under nitrogen atmosphere. The solution was chilled, and the solid was collected, washed with n-PrOH and dried in vacuo to provide title compound (2.00 g). ESI-MS (EI+, m/z): 270.16.

2. Synthesis of compound int. 15-2. To a solution of int. 15-1 (200 mg, 0.65 mmol) in methanol (4 mL), methoxy-sodium (46 mg, 0.85 mmol) was added at room temperature, dimethyl oxalate (150 mg, 1.27 mmol) was followed at room temperature. The mixture was heated up to 75° C. for 6 h. The resultant mixture was filtered and the filter cake was collected to give title compound (160 mg). ESI-MS (EI+, m/z): 338.17.

3. Synthesis of compound int. 15. To a solution of methyl 4-amino-6-((4-phenoxyphenyl)amino)-1,3,5-triazine-2-carboxylate (100 mg, 0.30 mmol) in THF (6 mL) was added lithium aluminum deuteride (20 mg, 0.48 mmol) at 0° C. The reaction mixture was allowed to warm to 25° C. for 2 h. The reaction mixture was quenched with saturated aqueous sodium sulfate at 0° C. The mixture was filtered, the filtrate was concentrated under reduced pressure to give desired product (170 mg). ESI-MS (EI+, m/z): 312.15.

### Example 16. Preparation of Compound Int. 16

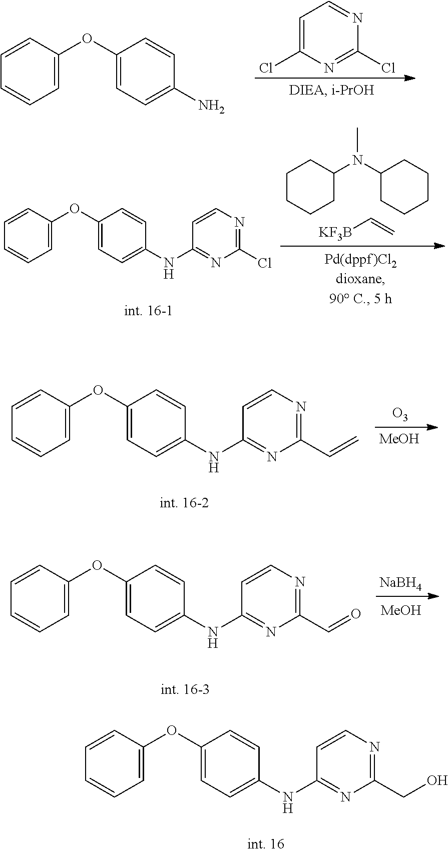

### Method 1

1. Synthesis of compound int. 16-1. A mixture of 2,4-dichloropyrimidine (10.0 g, 63.1 mmol), 4-phenoxyanilin (14.9 g, 80.6 mg) and DIEA (26.0 g, 201 mmol) in i-PrOH (100 mL) was stirred at 80° C. for 3 h. The reaction was concentrated, then the residue was purified by column chromatography to afford title compound (16.0 g). ¹H NMR (400 MHz, DMSO-d₆) δ 10.01 (s, 1H), 8.14 (d, J=6.0 Hz, 1H), 7.58 (d, J=8.4 Hz, 2H), 7.41-7.35 (m, 2H), 7.13 (d, J=7.2 Hz, 1H), 7.05 (d, J=8.8 Hz, 2H), 7.02-6.97 (m, 2H), 6.72 (d, J=6.0 Hz, 1H).

2. Synthesis of compound int. 16-2. A solution of 2-chloro-N4-(4-phenoxyphenyl)pyrimidine-4,6-diamine (5.0 g, 16.0 mmol), Pd(dppf)Cl₂ (1.17 g, 1.6 mmol), N-cyclohexyl-N-methylcyclohexanamine (9.3 g, 48 mmol) and potassium salt potassium vinyltrifluoroborate (8.6 g, 64 mmol) in dioxane (100 mL) was stirred at 100° C. for 4 h under nitrogen atmosphere. The reaction was purified by Prep-HPLC to afford title compound (300 mg). ESI-MS (EI⁺, m/z): 290.10.

3. Synthesis of compound int. 16-3. A solution of 2-ethenyl-N-(4-phenoxyphenyl)pyrimidin-4-amine (500 mg, 1.7 mmol) in MeOH (15 mL) was stirred at −78° C. for 30 seconds under O₃ atmosphere. The solution was used for next step directly. ESI-MS (EI⁺, m/z): 292.10.

4. Synthesis of compound int. 16. To a solution of 4-amino-6-[(4-phenoxyphenyl)amino]pyrimidine-2-carbaldehyde (500 mg, 1.63 mmol) in MeOH (15 mL) was added sodium borohydride (154 mg, 4.08 mmol) at 0° C. The resulting solution was stirred at room temperature for 3 h, then concentrated. The residue was purified by column chromatography to afford title compound. ESI-MS (EI+, m/z): 294.10.

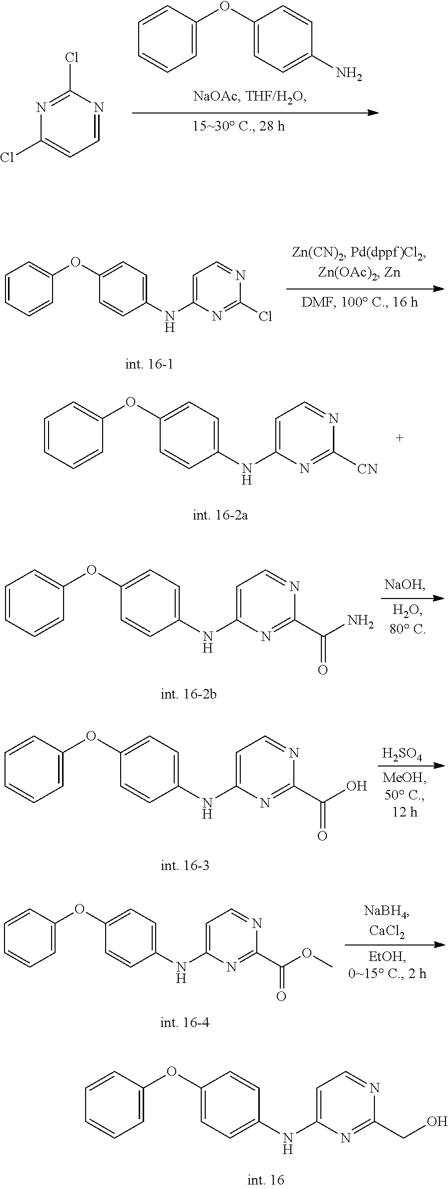

### Method 2

1. Synthesis of compound int. 16-1. To a mixture of 2,4-dichloropyrimidine (1.69 g, 11.3 mmol) and NaOAc (1.77 g, 21.6 mmol) in THF (20 mL) and H₂O (20 mL) was added 4-phenoxyaniline (2.00 g, 10.8 mmol), the mixture was stirred at 15° C. for 12 h. The mixture was heated at 30° C. for 16 h. The aqueous phase was extracted with ethyl acetate (30 mL×3). The combined organic layers were washed with brine (20 mL×2) and dried over Na₂SO₄, filtered and concentrated. To the residue was added ethyl acetate (15 mL) and DCM (10 mL), filtered and collected the filter cake to give title compound (1.65 g).

2. Synthesis of compound int. 16-2a and int. 16-2b. To a solution of 2-chloro-N-(4-phenoxyphenyl)pyrimidin-4-amine (1.00 g, 3.36 mmol), zinc powder (44 mg, 0.67 mmol), Zn(CN)₂ (237 mg, 2.02 mmol) and Pd(dppf)Cl₂ (246 mg, 0.34 mmol) in DMF (20 mL) was added Zn(OAc)₂ (62 mg, 0.34 mmol), the mixture was heated at 100° C. and stirred for 16 h under N₂ atmosphere. The aqueous phase was extracted with ethyl acetate (10 mL×3) and H₂O (5 mL). The residue was concentrated. The crude product was purified by column chromatography to give 4-((4-phenoxyphenyl)amino)pyrimidine-2-carbonitrile (160 mg) and 4-((4-phenoxyphenyl)amino)pyrimidine-2-carboxamide (580 mg).

3. Synthesis of compound int. 16-3. A mixture of 4-(4-phenoxyanilino)pyrimidine-2-carboxamide (570 mg, 1.86 mmol) and 4-(4-phenoxyanilino)pyrimidine-2-carbonitrile (150 mg, 0.52 mmol) in 2M NaOH aqueous solution (0.2 M, 12 mL) was heated at 100° C. for 12 h. To the mixture was added HCl aqueous solution (2M) to adjusted pH=5-6, filtered and collected filter cake. The residue was purified by Prep-HPLC to give title compound (255 mg).

4. Synthesis of compound int. 16-4. To a solution of 4-(4-phenoxyanilino)pyrimidine-2-carboxylic acid (130 mg, 0.42 mmol) in MeOH (1 mL) was added H₂SO₄ (0.1 mL) dropwise, the mixture was stirred at 50° C. for 12 h. To the mixture was added saturated aqueous NaHCO₃ (2 mL) and ethyl acetate (10 mL), the aqueous phase was extracted with ethyl acetate (10 mL×3). The combined organic layers were washed with brine (5 mL) and dried over Na₂SO₄, filtered and concentrated to give title compound (136 mg crude).

5. Synthesis of compound int. 16. To a solution of methyl 4-((4-phenoxyphenyl)amino)pyrimidine-2-carboxylate (135 mg crude) and CaCl₂ (28 mg, 0.25 mmol) in EtOH (3 mL) was added NaBH₄ (64 mg, 1.68 mmol) at 0° C., the mixture was stirred at 15° C. for 2 h. To the mixture was added H₂O (5 mL) and ethyl acetate (10 mL), the aqueous phase was extracted with ethyl acetate (10 mL×3). The combined organic layers were washed with brine (5 mL) and dried over Na₂SO₄, filtered and concentrated to title compound (130 mg crude).

### Example 17. Preparation of Compound Int. 17

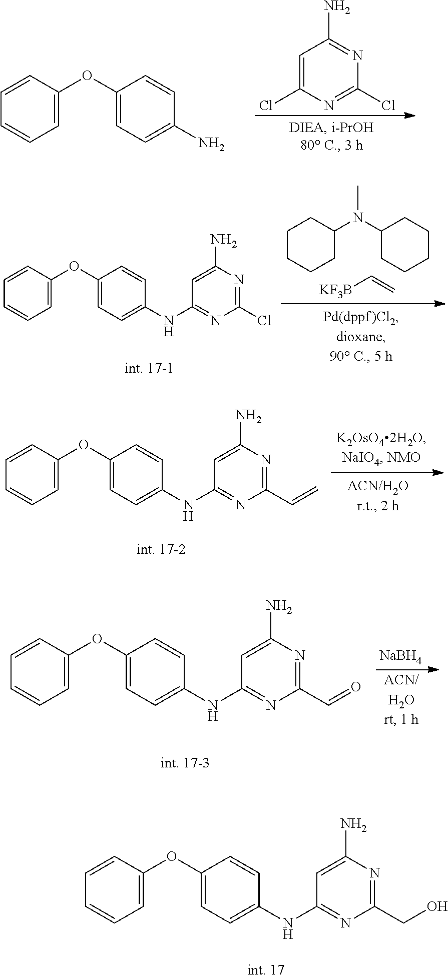

1. Synthesis of compound int. 17-1. A solution of 2,4-dichloro-7H-pyrrolo[2,3-d]pyrimidine (10.0 g, 54 mmol), 2,6-dichloropyrimidin-4-amine (8.85 g, 54.0 mmol) and DIPEA (20.9 g, 162 mmol) in i-PrOH (100 mL) was stirred at 80° C. for 3 h. The solution was poured into water (500 mL), filtered and concentrated to give the crude title product (15.0 g). ¹H NMR (400 MHz, DMSO-d₆) δ 9.56 (s, 1H), 7.75-7.64 (m, 2H), 7.41-7.27 (m, 2H), 7.14-7.03 (m, 1H), 7.00-6.90 (m, 4H), 5.97 (s, 1H).

2. Synthesis of compound int. 17-2. A mixture of 2-chloro-N⁴-(4-phenoxyphenyl)pyrimidine-4,6-diamine (5.0 g, 16 mmol), Pd(dppf)₂Cl₂ (1.17 g, 1.6 mmol), N-cyclohexyl-N-methylcyclohexanamine (9.37 g, 48.0 mmol) and potassium vinyltrifluoroborate (6.42 g, 48.0 mmol) in dioxane (100 mL) was stirred at 90° C. under N₂ atmosphere for 5 h. The reaction mixture was concentrated. The residue was purified by column chromatography to give title compound (2.0 g). ¹H NMR (400 MHz, DMSO-d₆) δ 10.28 (s, 1H), 8.34 (s, 2H), 7.69-7.58 (m, 2H), 7.43-7.35 (m, 2H), 7.13 (t, J=7.2 Hz, 1H), 7.08-6.95 (m, 4H), 6.63 (dd, J=17.8, 11.2 Hz, 11H), 6.33-6.18 (m, 2H), 5.86 (d, J=11.2 Hz, 1H), 3.17 (s, 1H).

3. Synthesis of compound int. 17-3. A solution of N⁴-(4-phenoxyphenyl)-2-vinylpyrimidine-4,6-diamine (1.0 g, 3.29 mmol), K₂OsO₄·2H₂O (112 mg, 0.76 mmol), 4-methylmorpholin-4-ium-4-olate (1.92 g, 60.9 mmol), NaIO₄ (3.51 g, 16.4 mmol) in H₂O (30 mL) and ACN (6 mL) was stirred at room temperature for 2 h. The reaction mixture was filtered and the filtrate was used directly for next step.

4. Synthesis of compound int. 17. To a solution of 4-amino-6-((4-phenoxyphenyl)amino)pyrimidine-2-carbaldehyde (500 mg, 1.63 mmol) in ACN (10 mL) was added sodium borohydride (185 mg, 4.90 mmol). The mixture was stirred at room temperature for 2 h, then concentrated. The residue was purified by column chromatography to give title compound (300 mg). ¹H NMR (400 MHz, DMSO-d₆) δ 8.89 (s, 1H), 8.14 (s, 1H), 7.87-7.73 (m, 2H), 7.43-7.21 (m, 2H), 7.05 (dd, J=8.0, 6.8 Hz, 1H), 6.91 (td, J=6.4, 1.7 Hz, 4H), 6.51 (s, 2H), 6.07 (s, 1H), 4.24 (s, 2H).

### Example 18. Preparation of Compound Int. 18

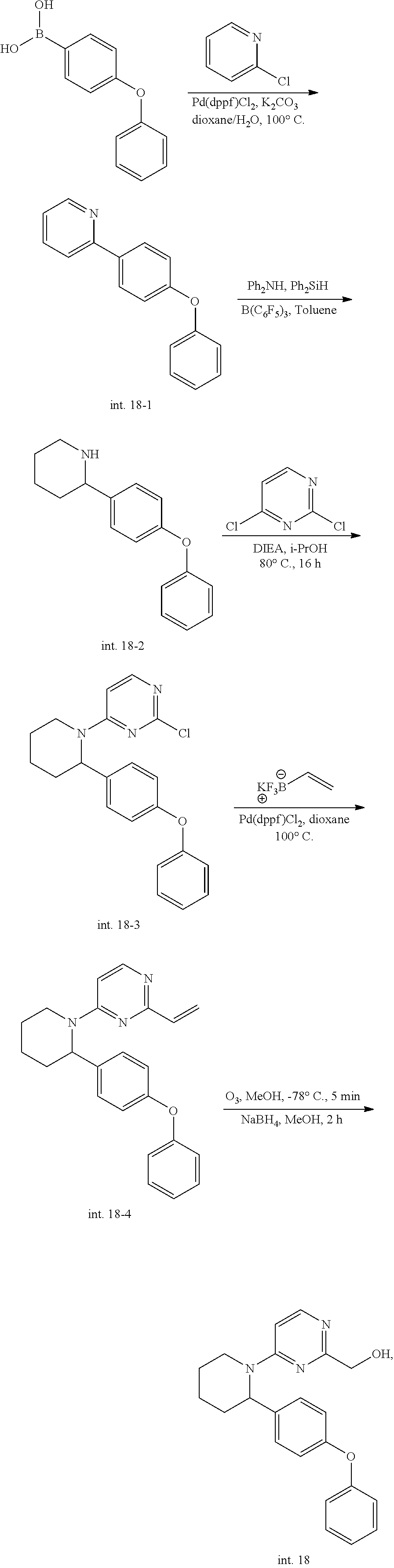

1. Synthesis of compound int. 18-1. A solution of 4-phenoxyphenylboronic acid (4.5 mg, 21.1 mmol), 2-chloropyridine (2.00 g, 17.6 mmol), Pd(dppf)Cl₂ (1.29 g, 1.76 mmol) and K₂CO₃ (7.30 g, 52.8 mmol) in dioxane (50 mL) and H₂O (5 mL) was stirred at 100° C. for 4 h under N₂ atmosphere. The organic layer was dried over sodium sulfate, concentrated. The residue was purified by column chromatography to give title compound (4.0 g).

2. Synthesis of compound int. 18-2. A solution of 2-(4-phenoxyphenyl)pyridine (2 g, 8.09 mmol), tris(perfluorophenyl)borane (414.06 mg, 0.81 mmol), diphenylsilane (7.45 g, 40.4 mmol) and diphenylamine (5.47 g, 32.4 mmol) in toluene (20 mL) was stirred at 100° C. for 16 h. The organic layer was dried over sodium sulfate and concentrated. The residue was purified by column chromatography to give title compound (1.9 g). ESI-MS (EI⁺, m/z): 254.25.

3. Synthesis of compound int. 18-3. A solution of 2-(4-phenoxyphenyl)piperidine (2.30 g, 9.08 mmol), 2,4-dichloropyrimidine (1.49 g, 9.99 mmol) and DIPEA (3.52 g, 27.2 mmol) in i-PrOH (20 mL) was stirred at 80° C. for 16 h. The organic layer was dried over sodium sulfate and concentrated. The residue was purified by column chromatography to give title compound (2.6 g).

4. Synthesis of compound int. 18-4. A solution of 2-chloro-4-[2-(4-phenoxyphenyl)piperidin-1-yl]pyrimidine (2.50 g, 6.83 mmol), Pd(dppf)Cl₂ (501 mg, 0.68 mmol), N-Methyldicyclohexylamine (4.00 g, 20.5 mmol) and potassium trifluoro(vinyl)borate (3.66 g, 27.3 mmol) in dioxane (20 mL) was stirred at 100° C. for 4 h under nitrogen. The residue was purified by column chromatography to give title compound (1.7 g). ESI-MS (EI⁺, m/z): 358.25.

5. Synthesis of compound int. 18. A solution of 2-ethenyl-4-[2-(4-phenoxyphenyl)piperidin-1-yl]pyrimidine (500 mg, 1.4 mmol) in MeOH (10 mL) was stirred at −78° C. under ozone for 5 min. Sodium borohydride (52.6 mg, 1.39 mmol) was added to the solution at 0° C. The solution was stirred at 25° C. for 1 h. The residue was purified by column chromatography to give title compound (100 mg).

### Example 19. Preparation of Compound Int. 19

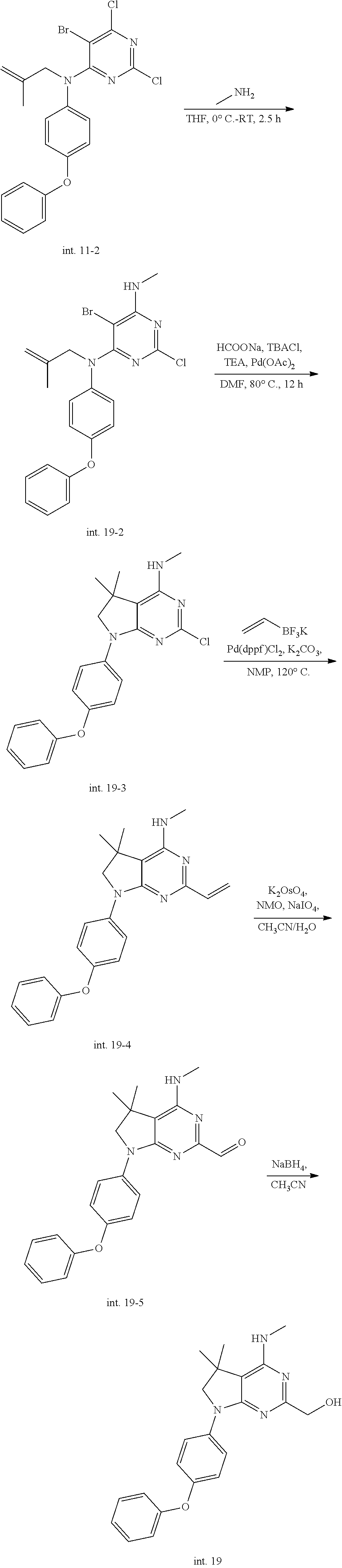

1. Synthesis of compound int. 19-2. To a solution of 5-bromo-2,6-dichloro-N-(2-methylallyl)-N—(4-phenoxyphenyl)pyrimidin-4-amine (7.70 g, 8.28 mmol) in THF (40 mL) was added methanamine (514 mg, 16.6 mmol) at 0° C. under N₂ atmosphere, then the reaction was stirred at 25° C. for 2.5 h. The solvent was evaporated and the residue was purified by column chromatography to afford title compound (4.40 g, purity: 90%). ESI-MS (EI⁺, m/z): 459.10. ¹H NMR (400 MHz, DMSO-d₆) δ 7.40-7.33 (m, 2H), 7.30 (q, J=4.4 Hz, 1H), 7.10 (t, J=7.5 Hz, 1H), 7.04-6.89 (m, 6H), 4.95-4.78 (m, 2H), 4.46 (s, 2H), 2.83 (d, J=4.5 Hz, 3H), 1.72 (s, 3H).

2. Synthesis of compound int. 19-3. A mixture of 5-bromo-2-chloro-N4-methyl-N6-(2-methylallyl)-N6-(4-phenoxyphenyl)pyrimidine-4,6-diamine (4.40 g, 9.57 mmol), sodium formate (690 mg, 10.1 mmol), tetrabutylammonium chloride (2.66 g, 9.57 mmol), TEA (2.48 g, 24.5 mmol), (acetyloxy)palladio acetate (430 mg, 1.91 mmol) in DMF (20 mL) was stirred at 80° C. for 12 h under N₂ atmosphere. Water (50 mL) was added and the mixture was extracted with EA (30 mL×3). The organic layers were combined, washed with water (25 mL×6), dried and evaporated. The residue was purified by column chromatography to afford title compound (2.40 g, purity: 85%). ESI-MS (EI⁺, m/z): 381.20. ¹H NMR (400 MHz, DMSO-d₆) δ 7.70-7.58 (m, 2H), 7.40-7.30 (m, 2H), 7.12-7.01 (m, 3H), 7.00-6.90 (m, 2H), 6.41 (d, J=4.6 Hz, 1H), 3.73 (s, 2H), 2.85 (d, J=4.5 Hz, 3H), 1.38 (s, 6H).

3. Synthesis of compound int. 19-4. A mixture of 2-chloro-N,5,5-trimethyl-7-(4-phenoxyphenyl)-6,7-dihydro-5H-pyrrolo[2,3-d]pyrimidin-4-amine (88 mg, 0.23 mmol), potassium vinyltrifluoroborate, (154 mg, 1.15 mmol), Pd(dppf)Cl₂ (16.8 mg, 0.02 mmol), K₂CO₃ (95.4 mg, 0.69 mmol) in NMP (5 mL) was stirred at 120° C. for 12 h under N₂ atmosphere. Water (100 mL) was added, and the mixture was extracted with EA (100 mL×3). The organic layers were combined, washed with water (100 mL), dried and evaporated. The crude was purified by flash column chromatography to afford title compound (1.74 g, purity: 90%). ESI-MS (EI+, m/z): 373.3. ¹H NMR (400 MHz, DMSO-d₆) δ 7.89-7.79 (m, 2H), 7.42-7.29 (m, 2H), 7.13-7.00 (m, 3H), 7.00-6.90 (m, 2H), 6.55 (dd, J=17.3, 10.3 Hz, 1H), 6.37 (dd, J=17.2, 2.5 Hz, 1H), 6.04 (d, J=4.7 Hz, 1H), 5.52 (dd, J=10.3, 2.5 Hz, 1H), 3.67 (s, 2H), 2.91 (d, J=4.4 Hz, 3H), 1.39 (s, 6H).

4. Synthesis of compound int. 19-5. A mixture of N,5,5-trimethyl-7-(4-phenoxyphenyl)-2-vinyl-6,7-dihydro-5H-pyrrolo[2,3-d]pyrimidin-4-amine (1.64 g, 4.4 mmol), K₂OsO₄·2H₂O (154 mg, 0.44 mmol), 4-methylmorpholin-4-ium-4-olate (2.58 mg, 22.0 mmol), NaIO₄ (4.71 g, 22.0 mmol) in acetonitrile (80 mL)/Water (20 mL) was stirred at 25° C. for 8 h under N₂. The reaction mixture was filtered and the filtrate was concentrated to give the crude title compound (1.70 g, purity: 60%). ESI-MS (EI+, m/z): 375.25.

5. Synthesis of compound int. 19. A mixture of 5,5-dimethyl-4-(methylamino)-7-(4-phenoxyphenyl)-6,7-dihydro-5H-pyrrolo[2,3-d]pyrimidine-2-carbaldehyde (1.70 g, 4.54 mmol), NaBH₄ (343 mg, 9.08 mmol) in acetonitrile (80 mL) was stirred at 25° C. for 2 h under N₂ atmosphere. The reaction mixture was filtered. The filtrate was concentrated and the crude was purified by column chromatography to give title compound (717 mg, purity: 85%). ESI-MS (EI+, m/z): 377.20. ¹H NMR (400 MHz, DMSO-d_{b}) δ 7.86-7.74 (m, 2H), 7.41-7.28 (m, 2H), 7.07 (t, J=7.4 Hz, 1H), 7.04-6.98 (m, 2H), 6.98-6.90 (m, 2H), 6.09 (q, J=4.5 Hz, 1H), 4.60 (t, J=5.7 Hz, 1H), 4.30 (d, J=5.7 Hz, 2H), 3.68 (s, 2H), 2.90 (d, J=4.4 Hz, 3H), 1.38 (s, 6H).

### Example 20. Preparation of Compound Int. 20

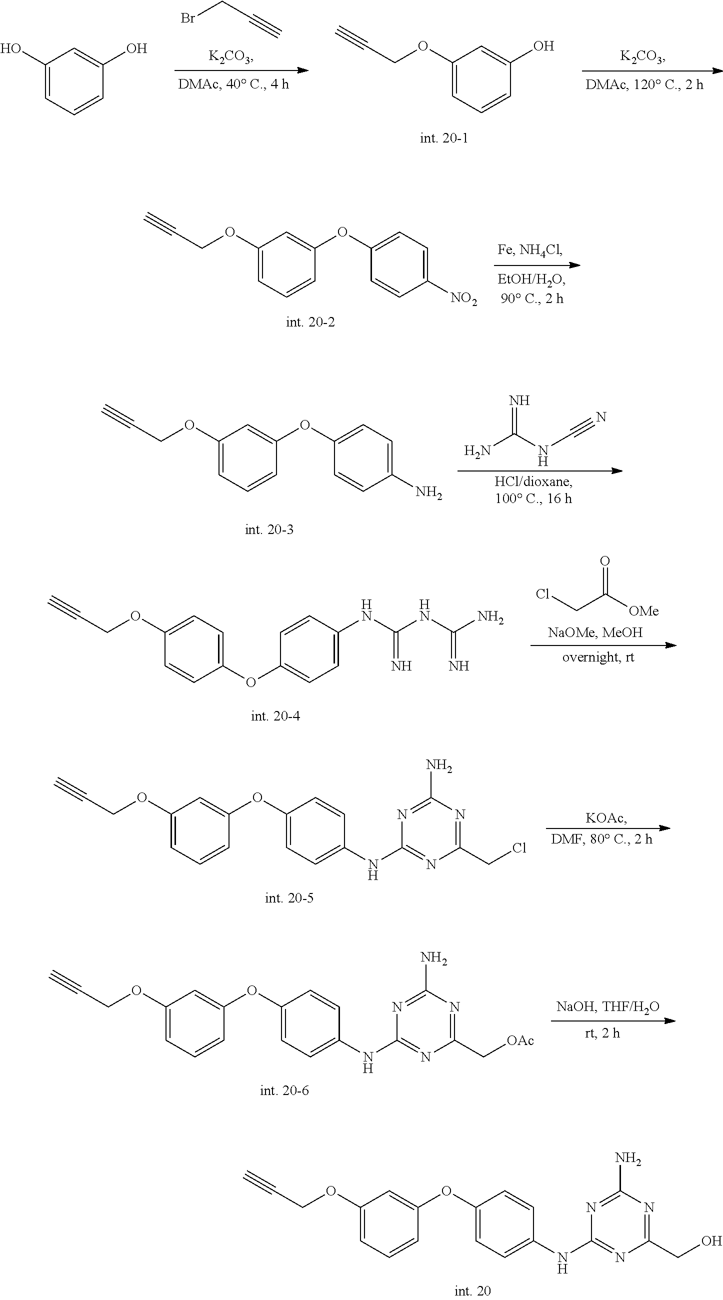

1. Synthesis of compound int. 20-1. To a solution of resorcinol (50.0 g, 454 mmol), K₂CO₃(188 g, 1.36 mol) in DMAc (50 mL) was added 3-bromoprop-1-yne (67.5 g, 454 mmol) and the mixture was stirred at 40° C. for 4 h under N₂ atmosphere. The mixture was diluted with EA (200 mL) and was washed with a solution of aqueous LiCl (150 mL×2). The organic layer was dried over sodium sulfate and concentrated. The crude material was purified by column chromatography to afford the title product (1.20 g). ESI-MS (EI+, m/z): 149.20.

2. Synthesis of compound int. 20-2. To a solution of 3-(prop-2-yn-1-yloxy)phenol (1.20 g, 8.1 mmol) and 4-fluoronitro benzene (143 mg, 8.1 mmol) in DMAc (10 mL) was added K₂CO₃ (1.68 g, 12.2 mmol) and the mixture was stirred at 120° C. for 2 h under N₂ atmosphere. The mixture was diluted with EA (150 mL), washed with a solution of aqueous LiCl (150 mL×2). The organic layer was dried over sodium sulfate and concentrated. The crude material was purified by column chromatography to afford title product (1.90 g). ¹H NMR (400 MHz, DMSO-d₆) δ 8.28-8.23 (m, 2H), 7.41 (t, J=8.2 Hz, 1H), 7.17-7.12 (m, 2H), 6.92 (m, J=8.4, 2.5, 0.9 Hz, 1H), 6.84 (t, J=2.4 Hz, 1H), 6.78 (m, J=8.1, 2.3, 0.8 Hz, 1H), 4.83 (d, J=2.4 Hz, 2H), 3.60 (t, J=2.4 Hz, 1H).

3. Synthesis of compound int. 20-3. To a solution of 1-(4-nitrophenoxy)-3-(prop-2-yn-1-yloxy)benzene (100 mg, 0.37 mmol), ammonium chloride (99.0 mg, 1.85 mmol) in EtOH (4 mL)/H₂O (1 mL) was added Fe (104 mg, 1.86 mmol) dropwise and the mixture was stirred at 90° C. for 2 h. The mixture was diluted with EA (150 mL) and was washed with aqueous NaCl solution (150 mL×2). The organic layer was dried and concentrated. The crude material was purified by column chromatography to afford title product. ESI-MS (EI⁺, m/z): 240.20.

4. Synthesis of compound int. 20-4. A solution of 4-[3-(prop-2-yn-1-yloxy)phenoxy]aniline (550 mg, 2.3 mmol) and dicyandiamide (193 mg, 2.3 mmol) in HCl/dioxane (6 mL) was stirred at 100° C. for 16 h. The mixture was centrifuged, the liquid was concentrated to afford title product (400 mg). ESI-MS (EI⁺, m/z): 324.20.

5. Synthesis of compound int. 20-5. To a solution of int. 20-4 (6.00 g, 7.42 mmol) and sodium methanolate (6.68 g, 37.1 mmol), methyl 2-chloroacetate (4.03 g, 37.1 mmol) and the mixture was stirred at room temperature for overnight under a N₂ atmosphere. The mixture was concentrated. The crude was purified by column chromatography to afford the title product (2.0 g). ESI-MS (EI⁺, m/z): 382.15.

6. Synthesis of compound int. 20-6. To a solution of 6-(chloromethyl)-N2-(4-(3-(prop-2-yn-1-yloxy)phenoxy)phenyl)-1,3,5-triazine-2,4-diamine (2.00 g, 5.24 mmol) in DMF (2 M) at 80° C. under a N₂ atmosphere was added potassium acetate (1.54 g, 15.7 mmol) and the mixture was stirred at 80° C. for 2 h. The mixture was diluted with EA (150 mL) and was washed with solution of aqueous NaCl (150 mL×2). The organic layer was dried and concentrated to afford title product (2.30 g). ESI-MS (EI+, m/z): 406.10.

7. Synthesis of compound int. 20. To a solution of (4-amino-6-((4-(3-(prop-2-yn-1-yloxy)phenoxy)phenyl)amino)-1,3,5-triazin-2-yl)methyl acetate (2.30 g, 5.67 mmol) in THF (18 mL)/H₂O (6 mL), sodium hydroxide (386 mg, 9.64 mmol) was added and the mixture was stirred at room temperature for 2 h. The mixture was adjusted pH to 4-5 and diluted with EA (150 mL), washed with a solution of aqueous NaCl (150 mL×2). The organic layer was dried and concentrated. The crude was purified by column chromatography to afford the title product. ESI-MS (EI+, m/z): 364.20. ¹H NMR (400 MHz, DMSO-d₆) δ 9.55 (s, 1H), 7.84-7.76 (m, 2H), 7.26 (t, J=8.2 Hz, 1H), 7.11-6.93 (m, 4H), 6.71 (dd, J=8.3, 2.4 Hz, 1H), 6.59 (t, J=2.4 Hz, 1H), 6.52 (dd, J=8.1, 2.3 Hz, 1H), 4.77 (d, J=2.4 Hz, 2H), 4.22 (s, 2H), 3.56 (t, J=2.4 Hz, 1H).

### Example 21. Preparation of Compound Int. 21

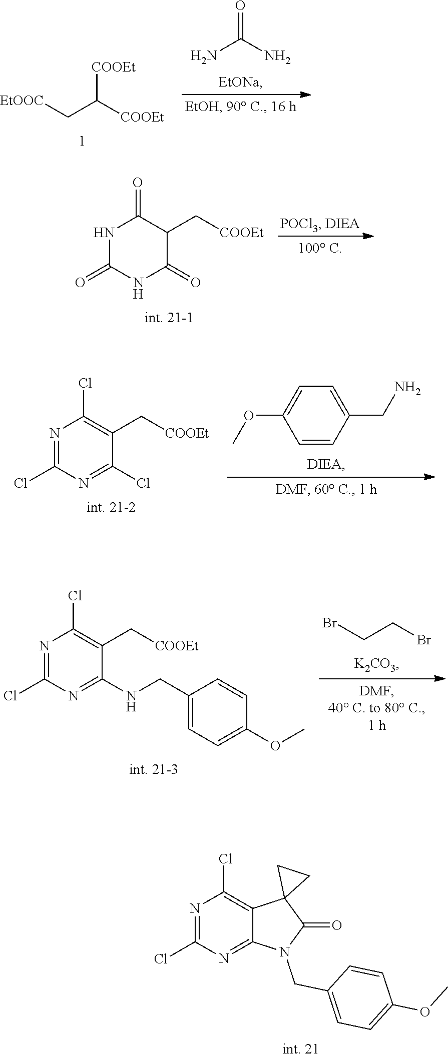

1. Synthesis of compound int. 21-1. To a solution of triethyl 1,1,2-ethanetricarboxylate (50.0 g, 203 mmol) in EtOH (120 mL) were added urea (12.2 g, 203 mmol) and sodium ethoxide (20.7 g, 305 mmol). The mixture was heated at 90° C. for 16 h, then diluted with water (100 mL), and extracted with EA (100 mL×4). The organic layer was dried and concentrated under reduced pressure to obtain title compound (25.0 g). ¹H NMR (400 MHz, DMSO-d₆) δ 11.30 (s, 1H), 4.03 (qd, J=7.2, 4.0 Hz, 3H), 1.98 (s, 2H), 1.19-1.15 (m, 3H).

2. Synthesis of compound int. 21-2. To a solution of (2,4,6-trioxo-hexahydro-pyrimidin-5-yl)-acetic acid ethyl ester (5.0 g, 23.3 mmol) in POCl₃ (50 mL) was added DIPEA (10 mL) dropwise at 0° C. The mixture was heated at 100° C. for 3 h. The mixture was concentrated under reduced pressure. The residue was diluted with EA (50 mL), quenched with saturated aqueous solution of NaHCO₃ (100 mL), extracted with EA (20 mL×2). The organic phase was dried and concentrated. The residue was purified by column chromatography to afford the title product (3.0 g). ¹H NMR (400 MHz, DMSO-d₆) δ 4.15 (d, J=7.2 Hz, 2H), 3.99 (s, 2H), 1.20 (t, J=7.2 Hz, 3H).

3. Synthesis of compound int. 21-3. To a solution of ethyl 2-(2,4,6-trichloropyrimidin-5-yl)acetate (4.00 g, 14.8 mmol) in DMF (15 mL) were added 4-methoxybenzylamine (2.24 g, 16.3 mmol) and DIPEA (2.30 g, 17.8 mmol) at 25° C. The mixture was heated to 60° C. for 1 h. The mixture was diluted with EA (50 mL) and was washed with a solution of aqueous NaHCO₃ (50 mL×2). The organic layer was dried and concentrated to obtain title compound (4.7 g, purity: 60%).

4. Synthesis of compound int. 21. To a solution of ethyl 2-(2,4-dichloro-6-{[(4-methoxyphenyl)methyl]amino}pyrimidin-5-yl)acetate (10.0 g, 27 mmol) and K₂CO₃ (33.2 g, 270 mmol) in DMF (50 mL) was added DBE (10.1 g, 54 mmol) dropwise under N₂ atmosphere at 40° C. After the addition was over, the reaction mixture was heated to 80° C. for 1 h. The mixture was diluted with EA (50 mL) and was washed with a solution of aqueous NaHCO₃ (5×100 mL). The organic layer was dried and concentrated. The residue was purified by column chromatography to afford the title product (3.0 g). ESI-MS (EI+, m/z): 349.7. ¹H NMR (400 MHz, DMSO-d₆) δ 7.30-7.22 (m, 2H), 6.92-6.84 (m, 2H), 4.85 (s, 2H), 3.71 (s, 3H), 2.12 (q, J=4.4 Hz, 2H), 1.69 (q, J=4.3 Hz, 2H).

### Example 22. Preparation of Compound Int. 22

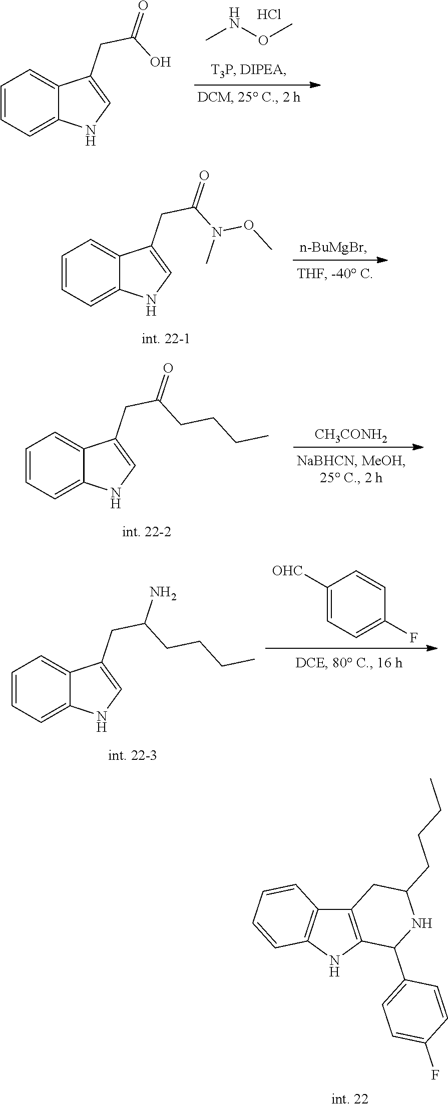

1. Synthesis of compound int. 22-1. To a solution of 2-(1H-indol-3-yl)acetic acid (8.0 g, 45.7 mmol), O,N-dimethyl-hydroxylamine hydrochloride (4.90 g, 50.2 mmol), T₃P (43.6 g, 68.5 mmol), DIPEA (16.2 g, 126 mmol) in DCM (100 mL) was stirred at 25° C. for 2 h. The solution was poured into water, extracted with EA, washed with brine, concentrated in vacuo to get title product (9.9 g). ESI-MS (EI⁺, m/z): 219.2.

2. Synthesis of compound int. 22-2. To a solution of 2-(1H-indol-3-yl)-N-methoxy-N-methylacetamide (10.0 g, 45.8 mmol) in THF (100 mL) was added bromo(butyl)magnesium (22.2 g, 137 mmol) at −40° C. and stirred for 16 h. The solution was quenched with NH₄Cl (aq.) at −78° C., filtered, extracted with EA. The organic phase was concentrated. The residue was purified by column chromatography to get title compound (2.6 g). ¹H NMR (400 MHz, DMSO-d₆) δ 10.94 (s, 1H), 7.43 (dd, J=7.9, 1.1 Hz, 1H), 7.36 (dt, J=8.2, 1.0 Hz, 1H), 7.23 (d, J=2.3 Hz, 1H), 7.07 (ddd, J=8.1, 6.9, 1.2 Hz, 1H), 6.98 (ddd, J=8.0, 7.0, 1.1 Hz, 1H), 3.77 (s, 2H), 2.46 (t, J=7.3 Hz, 2H), 1.46-1.35 (m, 2H), 1.24-1.12 (m, 2H), 0.79 (t, J=7.3 Hz, 3H).

3. Synthesis of compound int. 22-3. A solution of 1-(1H-indol-3-yl)hexan-2-one (2.60 g, 12.1 mmol), acetamide (14.0 g, 181 mmol) in MeOH (30 mL) was stirred at 25° C. for 2 h, then sodium cyanoborohydride (1.52 g, 24.2 mmol) was added and stirred for 16 h. TLC showed the reaction was completed. The solution was quenched with NH₄Cl (aq.), extracted with EA, washed with brine, concentrated in vacuo to get title compound (2.5 g). ¹H NMR (400 MHz, DMSO-d₆) δ 10.96 (s, 1H), 7.55 (d, J=7.8 Hz, 1H), 7.37 (d, J=8.1 Hz, 1H), 7.23 (d, J=2.4 Hz, 1H), 7.09 (t, J=7.5 Hz, 1H), 7.01 (t, J -7.4 Hz, 1H), 2.92 (d, J=6.6 Hz, 2H), 1.61-1.09 (m, 8H), 0.84 (t, J=7.1 Hz, 3H).

4. Synthesis of compound int. 22. A solution of 4-fluorobenzaldehyde (1.23 g, 9.91 mmol), 1-(1H-indol-3-yl)hexan-2-amine (1.95 g, 9.01 mmol), trifluoroacetic acid-D (2.06 g, 18.0 mmol) in DCE (40 mL) was stirred at 80° C. for 16 h. The solution was added NaHCO₃ (aq.), extracted with DCM, washed with water, concentrated in vacuo. The residue was purified by silica gel column to get title compound (1.1 g). ESI-MS (EI⁺, m/z): 323.18. ¹H NMR (400 MHz, DMSO-d₆) δ 10.16 (s, 1H), 7.43-7.35 (m, 3H), 7.16 (ddd, J=12.1, 8.3, 3.4 Hz, 3H), 7.00-6.88 (m, 2H), 5.14 (s, 1H), 3.00-2.90 (m, 1H), 2.81-2.72 (m, 1H), 2.46-2.29 (m, 2H), 1.56 (dd, J=13.2, 7.1 Hz, 2H), 1.43 (dd, J=8.7, 5.5 Hz, 2H), 1.33 (q, J=7.3 Hz, 2H), 0.91 (t, J=7.2 Hz, 3H).

### Example 23. Preparation of Compound Int. 23

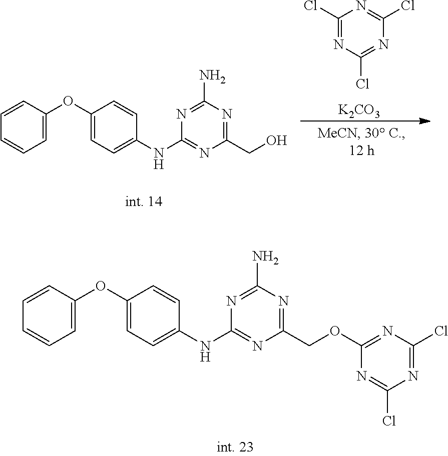 [SMILES: Nc1nc(CO)nc(Nc2ccc(Oc3ccccc3)cc2)n1 ; "K2CO3" ; Clc1nc(Cl)nc(Cl)n1 ; Nc1nc(COc2nc(Cl)nc(Cl)n2)nc(Nc2ccc(Oc3ccccc3)cc2)n1]

To a solution of (4-amino-6-((4-phenoxyphenyl)amino)-1,3,5-triazin-2-yl)methanol (2.0 g, 6.47 mmol) and K₂CO₃ (1.79 g, 12.9 mmol) in MeCN (15 mL) was added a solution of 2,4,6-trichloro-1,3,5-triazine (1.79 g, 9.70 mmol) in MeCN (25 mL) dropwise. The mixture was stirred at 30° C. for 12 h, then concentrated. The residue was purified by column chromatography to title product (470 mg). ¹H NMR (400 MHz, DMSO-d₆) δ 11.19 (s, 1H), 7.77-7.64 (m, 2H), 7.41-7.32 (m, 2H), 7.14-7.06 (m, 1H), 7.00-6.92 (m, 4H), 5.19 (s, 2H).

### Example 24. Preparation of Compound Int. 24

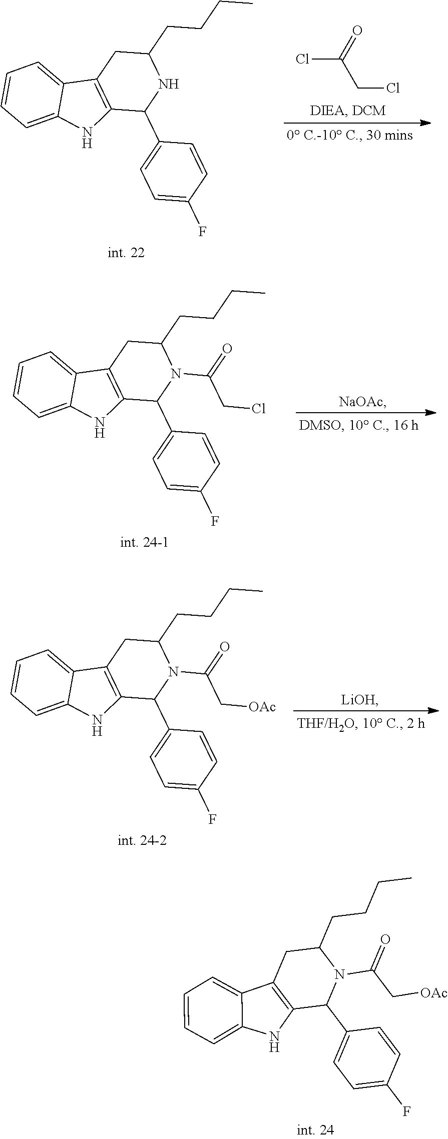

1. Synthesis of compound int. 24-1. To a solution of (1S,3S)-3-butyl-1-(4-fluorophenyl)-2,3,4,9-tetrahydro-1H-pyrido[3,4-b]indole (300 mg, 0.93 mmol) and DIEA (241 mg, 1.86 mmol) in DCM (10 mL) was added 2-chloroacetyl chloride (158 mg, 1.40 mmol) dropwise at 0° cand stirred at 10° C. for 30 min. The reaction mixture was concentrated. The residue was purified by column chromatography to give title compound (223 mg). ¹H NMR (400 MHz, DMSO-d₆) δ 11.09 (s, 1H), 7.50 (d, J=7.8 Hz, 1H), 7.43 7.31 (m, 3H), 7.24-7.08 (m, 3H), 7.04-7.00 (m, 1H), 6.93 (s, 1H), 4.75-4.47 (m, 2H), 4.32 (t, J=7.6 Hz, 1H), 3.13 (dd, J=15.6, 6.4 Hz, 1H), 2.82 (d, J=15.6 Hz, 1H), 1.39-1.30 (m, 2H), 1.17-1.05 (m, 1H), 0.85 (d, J=15.6 Hz, 1H), 0.72-0.46 (m, 5H).

2. Synthesis of compound int. 24-2. To a solution of 1-((1S,3S)-3-butyl-1-(4-fluorophenyl)-1,3,4,9-tetrahydro-2H-pyrido[3,4-b]indol-2-yl)-2-chloroethan-1-one (223 mg, 0.56 mmol) in DMSO (10 mL) was added NaOAc (138 mg, 1.68 mmol) and the mixture was stirred at 10° C. for 16 h. Water (30 mL) was added to reaction mixture and extracted with EtOAc (30 mL×2). The combined organic phases were washed with brine (30 mL), dried over anhydrous Na₂SO₄, filtered and concentrated to give title compound (268 mg crude). ESI-MS (EI⁺, m/z): 423.2 [M+H]⁺. ¹H NMR (400 MHz, CDCl₃) δ: 7.94 (s, 1H), 7.59-7.41 (m, 3H), 7.35 (d, J=8.0 Hz, 1H), 7.25-7.10 (m, 2H), 6.96 (dd, J=18.0, 9.6 Hz, 3H), 4.86 (dd, J=78.6, 14.4 Hz, 2H), 4.04 (d, J=7.2 Hz, 1H), 3.12 (dd, J=15.4, 6.4 Hz, 1H), 2.89 (d, J=15.4 Hz, 1H), 2.22 (s, 3H), 1.45-1.40 (m, 2H), 1.10-0.90 (m, 2H), 0.84-0.68 (m, 2H), 0.60 (t, J=7.2 Hz, 3H).

3. Synthesis of compound int. 24. To a solution of 2-((1S,3S)-3-butyl-1-(4-fluorophenyl)-1,3,4,9-tetrahydro-2H-pyrido[3,4-b]indol-2-yl)-2-oxoethyl acetate (268 mg, 0.63 mmol) in THF (4 mL) and H₂O (1 mL) was added LiOH·H₂O (133 mg, 3.17 mmol). The mixture was stirred at 10° C. for 2 h. Water (30 mL) was added to reaction mixture and extracted with EtOAc (30 mL×2). The combined organic phases were washed with brine (30 mL), dried over anhydrous Na₂SO₄, filtered and concentrated to give title compound (280 mg crude). ESI-MS (EI⁺, m/z): 381.2 [M+H]⁺. ¹H NMR (400 MHz, CDCl₃) δ: 7.89 (s, 1H), 7.58-7.43 (m, 3H), 7.36 (d, J=8.0 Hz, 1H), 7.25-7.13 (m, 2H), 6.99 (dd, J=14.4, 6.4 Hz, 3H), 4.40-4.20 (m, 2H), 3.87 (dt, J=18.4, 4.4 Hz, 2H), 3.09 (dd, J=15.6, 6.4 Hz, 1H), 2.90 (d, J=15.6 Hz, 1H), 1.42 (h, J=6.4, 5.2 Hz, 2H), 1.06-0.71 (m, 4H), 0.60 (t, J=7.2 Hz, 3H).

### Example 25. Preparation of Compound Int. 25

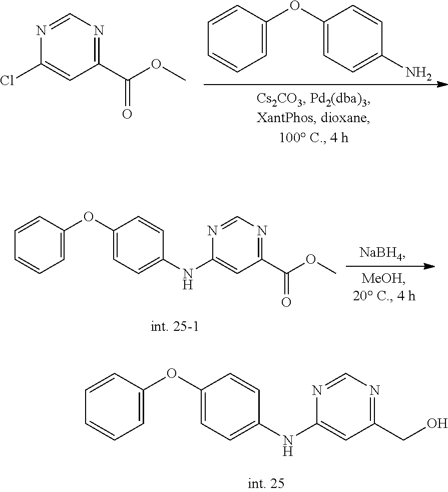

1. Synthesis of compound int. 25-1. To a solution of methyl 6-chloropyrimidine-4-carboxylate (1.00 g, 5.79 mmol) and 4-phenoxyaniline (1.07 g, 5.79 mmol) in dioxane (30 mL) was added Cs₂CO₃ (3.78 g, 11.6 mmol), Pd₂(dba)₃ (531 mg, 0.58 mmol) and XantPhos (671 mg, 1.16 mmol) and stirred at 100° C. for 4 h. The reaction was filtered and concentrated to give a residue. The residue was purified by column chromatography to give title compound (235 mg).

2. Synthesis of compound int. 25. To a solution of methyl 6-((4-phenoxyphenyl)amino)pyrimidine-4-carboxylate (250 mg, 0.78 mmol) in MeOH (3 mL) was added NaBH₄ (147 mg, 3.89 mmol) at 20° C. and stirred at 20° C. for 4 h. After the reaction mixture was cooled to 0° C., the reaction mixture was quenched by addition of 10 mL of H₂O. The reaction was added water (10 mL) and extracted with ethyl acetate (3×20 mL). The organic layers were washed with brine (10 mL), dried over Na₂SO₄, filtered and concentrated to give a residue. The residue was purified by column chromatography to give title compound (136 mg). ¹H NMR (400 MHz, Chloroform-d) δ 8.53 (d, J=1.2 Hz, 1H), 7.33-7.26 (m, 2H), 7.24-7.19 (m, 3H), 7.09-7.04 (m, 1H), 7.03-6.92 (m, 5H), 6.54 (d, J=1.2 Hz, 1H), 4.52 (d, J=0.8 Hz, 2H).

### Example 26. Preparation of Compound Int. 26

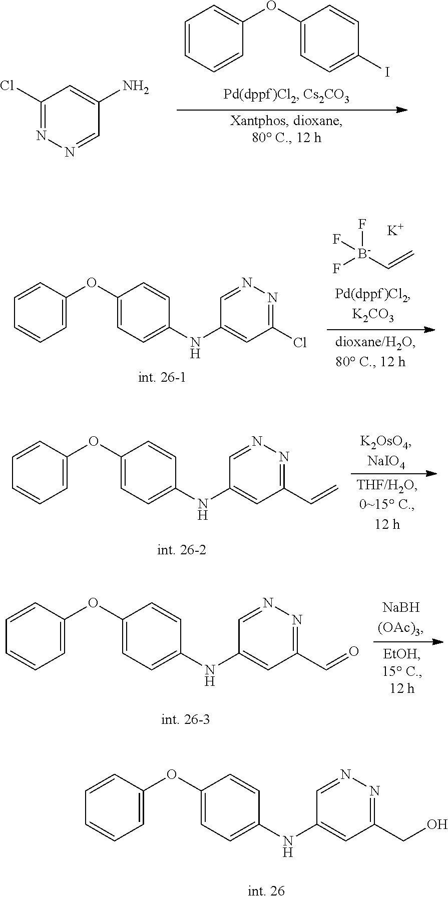

1. Synthesis of compound int. 26-1. To a mixture of 6-chloropyridazin-4-amine (440 mg, 3.40 mmol), XantPhos (393 mg, 0.68 mmol), Pd(dppf)Cl₂ (249 mg, 0.34 mmol), 1-iodo-4-phenoxy-benzene (1.01 g, 3.40 mmol) in dioxane (10 mL) was added Cs₂CO₃ (2.21 g, 6.79 mmol), the solution was heated at 80° C. and stirred for 12 h. To the mixture was added H₂O (30 mL) and ethyl acetate (50 mL), the aqueous phase was extracted with ethyl acetate (50 mL×3). The combined organic layers were washed with brine (30 mL) and dried over Na₂SO₄, filtered and concentrated. The crude product was purified by column chromatography and Prep-HPLC to give title compound (247 mg).

2. Synthesis of compound int. 26-2. To a mixture of 6-chloro-N-(4-phenoxyphenyl)pyridazin-4-amine (220 mg, 0.74 mmol), potassium trifluoro(vinyl)boranuide (99 mg, 0.74 mmol) and K₂CO₃ (204 mg, 1.48 mmol) in dioxane (5 mL) and H₂O (2 mL) was added Pd(dppf)Cl₂ (54 mg, 0.07 mmol), the mixture was heated at 80° C. for 12 h under N₂ atmosphere. To the mixture was added H₂O (5 mL) and ethyl acetate (10 mL), the aqueous phase was extracted with ethyl acetate (10 mL×3). The combined organic layers were washed with brine (5 mL) and dried over Na₂SO₄, filtered and concentrated. The crude product was purified by column chromatography to give title compound (70 mg).

3. Synthesis of compound int. 26-3. To a solution of N-(4-phenoxyphenyl)-6-vinylpyridazin-4-amine (50 mg, 0.02 mmol) and K₂OsO₄·2H₂O (5 mg, 0.02 mmol) in THF (1 mL) and H₂O (0.4 mL) was added NaIO₄ (149 mg, 0.69 mmol) at 0° C., the mixture was stirred at 15° C. for 12 h. To the mixture was added H₂O (1 mL) and ethyl acetate (2 mL), the aqueous phase was extracted with ethyl acetate (2 mL×3). The combined organic layers were washed with brine (1 mL) and dried over Na₂SO₄, filtered and concentrated to give title compound (50 mg crude).

4. Synthesis of compound int. 26. To a solution of 5-((4-phenoxyphenyl)amino)pyridazine-3-carbaldehyde (50 mg crude) in EtOH (1 mL) was added NaBH(OAc)₃ (73 mg, 0.34 mmol), the mixture was stirred at 15° C. for 12 h. To the mixture was added H₂O (0.1 mL), the mixture was concentrated. The crude product was purified by column chromatography to give title compound (20 mg).

### Example 27. Preparation of Compound Int. 27

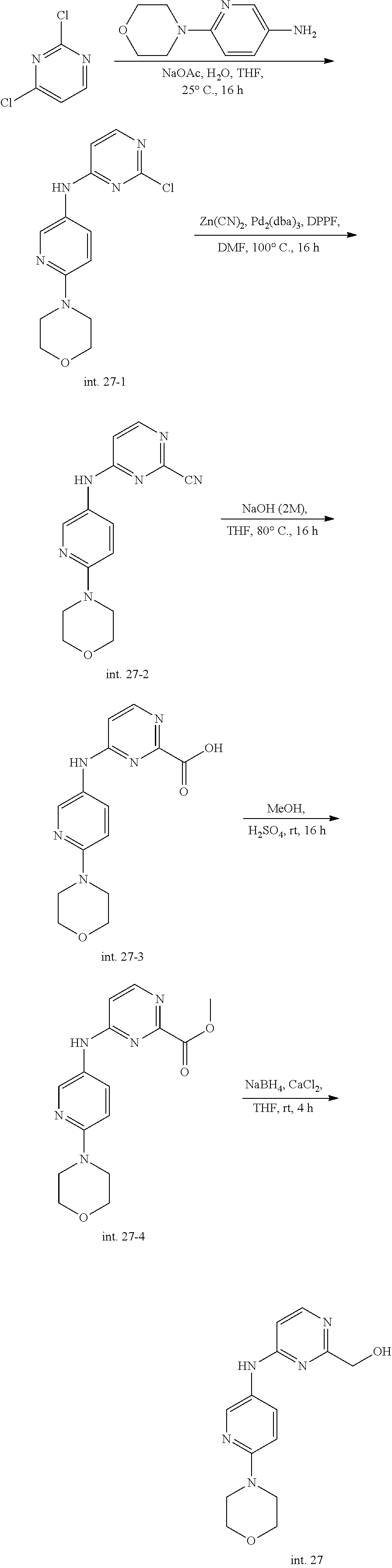

1. Synthesis of compound int. 27-1. To a solution of 2,4-dichloropyrimidine (2.49 g, 16.7 mmol) was added 6-morpholinopyridin-3-amine (2.0 g, 11.2 mmol) and NaOAc (3.04 g, 22.3 mmol) in the mixture of tetrahydrofuran (10 mL) and H₂O (10 mL) at 25° C. The reaction was stirred at 25° C. for 16 h. The mixture was diluted with water (10 mL), extracted with ethyl acetate (30 mL×2), washed with brine (20 mL×2), dried with Na₂SO₄, filtered and concentrated. The residue was purified by silica gel column to give title compound (2.8 g). ESI-MS (EI⁺, m/z): 292.1 [M+H]⁺.

2. Synthesis of compound int. 27-2. To a solution of 2-chloro-N-(6-morpholino-3-pyridyl)pyrimidin-4-amine (500 mg, 1.71 mmol), dicyanozinc (603 mg, 5.14 mmol) and cyclopentyl (diphenyl) phosphane, iron (95 mg, 0.17 mmol) in N,N-dimethylformamide (10 mL) was added (1E,4E)-1,5-diphenylpenta-1,4-dien-3-one, palladium (157 mg, 0.17 mmol) under N₂ atmosphere. The reaction was stirred at 100° C. for 16 h. The mixture was filtered and concentrated. The residue was purified by silica gel column to give title compound (450 mg, 60% purity). ESI-MS (EI⁺, m/z): 283.3 [M+H]⁺.

3. Synthesis of compound int. 27-3. To a solution of 4-[(6-morpholino-3-pyridyl)amino]pyrimidine-2-carbonitrile (400 mg, 1.42 mmol) in tetrahydrofuran (10 mL) was added in 2M aqueous NaOH (2M, 15 mL) at 25° C. The reaction was stirred at 80° C. for 16 h. The pH was adjusted to about 3 with 2M hydrochloric acid, and the aqueous phase was directly evaporated to give title compound (1.21 g crude). ESI-MS (EI⁺, m/z): 302.2 [M+H]⁺.

4. Synthesis of compound int. 27-4. 4-[(6-morpholino-3-pyridyl)amino]pyrimidine-2-carboxylic acid (200 mg, 0.66 mmol) was added to methanol (20 mL) and stirred for 10 min and then 0.5 mL of concentrated sulfuric acid (65 mg, 0.66 mmol) solution was dropped into the solution. The reaction was stirred at room temperature for 16 h. The pH of this mixture was adjusted to 9 with saturated aqueous NaHCO₃, then which was extracted with ethyl acetate (10 mL×2), washed with brine, dried with Na₂SO₄, filtered and concentrated. The mixture was purified with column chromatography to give title compound (50 mg, 61% purity). ESI-MS (EI⁺, m/z): 316.2 [M+H]⁺.

5. Synthesis of compound int. 27. To a solution of methyl 4-[(6-morpholino-3-pyridyl)amino]pyrimidine-2-carboxylate (50 mg, 0.16 mmol) in methanol (2 mL) was added NaBH₄ (24 mg, 0.63 mmol) and CaCl₂ (10 mg, 0.095 mmol) under ice cold bath. The reaction was stirred at 25° C. for 4 h. The mixture was filtered and concentrated. The residue was purified with column chromatography to give title compound (40 mg crude). ESI-MS (EI⁺, m/z): 288.2 [M+H]⁺.

### Example 28. Preparation of Compound Int. 28

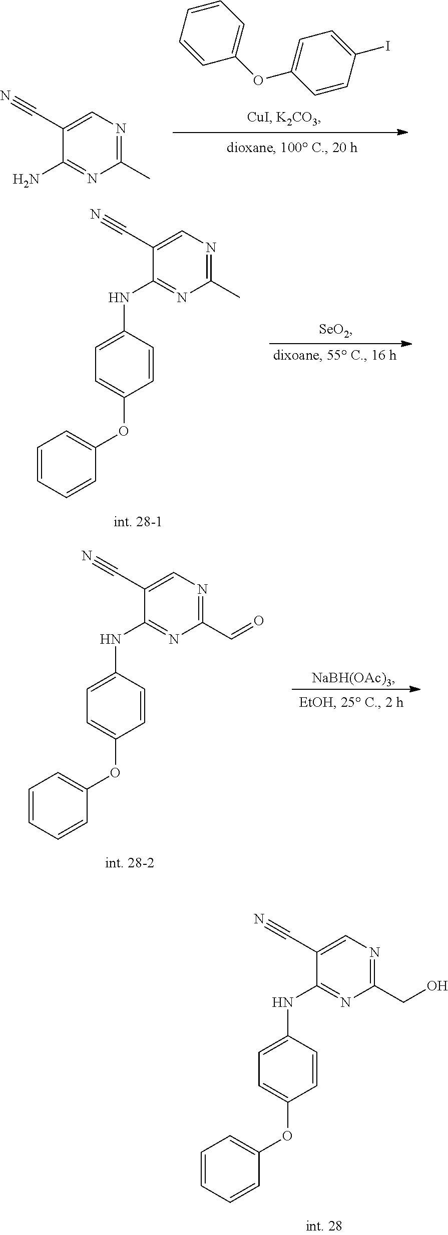

1. Synthesis of compound int. 28-1. The 4-amino-2-methyl-pyrimidine-5-carbonitrile (3.0 g, 22.4 mmol), CuI (2.13 g, 11.2 mmol) and K₂CO₃ (6.18 g, 44.7 mmol) were added to reaction flask under N₂ atmosphere. 1,4-dioxane (50 mL), 1-iodo-4-phenoxybenzene (5.96 g, 20.1 mmol) and N¹,N²-dimethylethane-1,2-diamine (1.97 g, 22.4 mmol) were added at room temperature. The reaction mixture was stirred at 100° C. for 20 h. The mixture was filtered and concentrated. The residue was purified by Prep-HPLC to give title compound (3.7 g). ESI-MS (EI⁺, m/z): 303.1[M+H]⁺.

2. Synthesis of compound int. 28-2. To a solution of 2-methyl-4-(4-phenoxyanilino)pyrimidine-5-carbonitrile (1.0 g, 3.31 mmol) in dioxane (15 mL) was added SeO₂ (477 mg, 4.30 mmol) under N₂ atmosphere. The reaction was stirred at 55° C. for 16 h. The reaction was added water (30 mL) and extracted with ethyl acetate (20 mL×3). The organic layers were concentrated

3. Synthesis of compound int. 28. To a solution of 2-formyl-4-(4-phenoxyanilino)pyrimidine-5-carbonitrile (90 mg, 0.28 mmol) in EtOH (2 mL) was added NaBH(OAc)₃ (121 mg, 0.576 mmol) under N₂ atmosphere. The reaction was stirred at 25° C. for 2 h. The reaction was added water (30 mL) and extracted with ethyl acetate (20 mL×3). The organic layers were concentrated, the residue was purified by column chromatography to give the desired product (50 mg, 73% purity). ESI-MS (EI⁺, m/z): 319.1 [M+H]⁺. to give a yellow oil. The crude was purified by column chromatography to give title compound (120 mg).

### Example 29. Preparation of Compound Int. 29

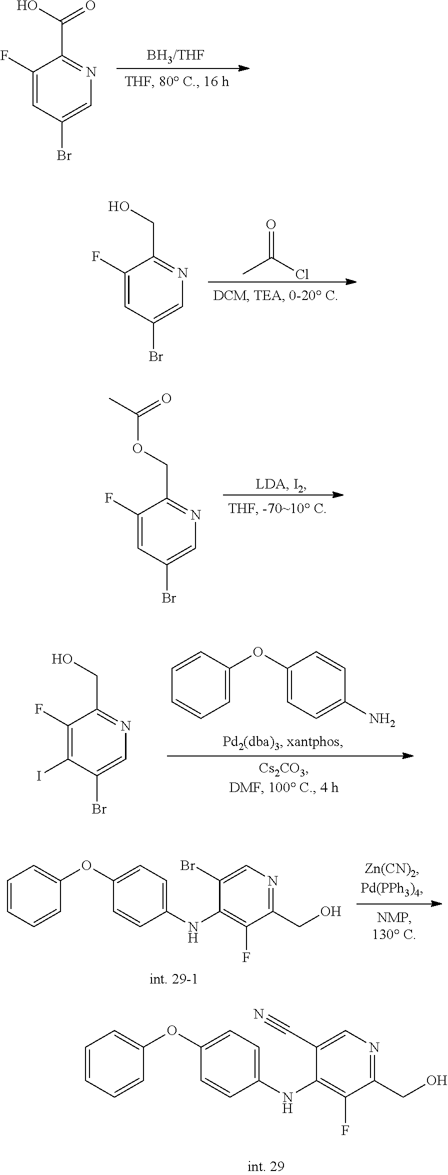

1. Synthesis of compound (5-Bromo-3-fluoro-2-pyridyl)methanol. To a solution of 5-bromo-3-fluoro-pyridine-2-carboxylic acid (9.0 g, 40.9 mmol) in THF (100 mL) was added borane/tetrahydrofuran solution (1M, 102 mL) dropwise at 0° C. The mixture was stirred at 15° C. for 16 h. The mixture was quenched by MeOH (20 mL) slowly and stirred at 80° C. for 1 h. The mixture was concentrated and the residue was purified by column chromatography to give title compound (3.4 g). ESI-MS (EI⁺, m/z): 206.0, 208.0 [M+H]⁺. ¹H NMR (400 MHz, CDCl₃) δ 8.69 (s, 1H), 7.69 (dt, J=1.6, 8.8 Hz, 1H), 5.33 (t, J=1.6 Hz, 2H).

2. Synthesis of compound (5-Bromo-3-fluoro-2-pyridyl)methyl acetate. To a solution of (5-bromo-3-fluoro-2-pyridyl)methanol (3.4 g, 16.5 mmol) in DCM (50 mL) were added acetyl chloride (2.0 mL) and Et₃N (6.9 mL) at 0° C. and the mixture was stirred at 20° C. for 16 h. The reaction mixture was concentrated. The residue was purified by column chromatography to give title compound (3.5 g). ¹H NMR (400 MHz, CDCl₃) δ 8.49 (dd, J=0.8, 2.0 Hz, 1H), 7.62 (dd, J=2.0, 8.8 Hz, 1H), 5.23 (s, 2H), 2.12 (s, 3H).

3. Synthesis of compound (5-Bromo-3-fluoro-4-iodo-2-pyridyl)methanol. To a solution of (5-bromo-3-fluoro-2-pyridyl)methyl acetate (3.1 g, 12.5 mmol) in THF (50 mL) was added LDA (2 M, 9.36 mL) at −70° C. dropwise under N₂ atmosphere. The mixture was stirred at −70° C. for 0.5 h. A solution of I₂ (3.81 g, 15.0 mmol) in THF (10 mL) was added dropwise at −70° C. The mixture was warmed to 10° C. slowly and stirred at 10° C. for 16 h. The reaction was quenched by water (100 mL), extracted with ethyl acetate (100 mL×2). The combined organic phases were washed with brine (100 mL), dried over anhydrous Na₂SO₄, filtered and concentrated. The residue was purified by column chromatography to give title compound (800 mg, 70% purity). ¹H NMR (400 MHz, CDCl₃) δ 8.46 (s, 1H), 4.78 (s, 2H).

4. Synthesis of compound int. 29-1. To a solution of (5-bromo-3-fluoro-4-iodo-2-pyridyl)methanol (800 mg, 1.69 mmol) and 4-phenoxyaniline (312 mg, 1.69 mmol) in DMF (20 mL) were added Pd₂(dba)₃ (154 mg, 0.17 mmol), XantPhos (98 mg, 0.17 mmol) and Cs₂CO₃ (1.10 g, 3.37 mmol). The mixture was stirred at 100° C. for 4 h under N₂ atmosphere. The solvent was removed and the residue was treated with ethyl acetate (30 mL). The mixture was filtered and the filtrate was concentrated to yield a residue which was purified by column chromatography to give title compound (200 mg). ESI-MS (EI⁺, m/z): 388.9, 390.9 [M+H]⁺. ¹H NMR (400 MHz, CDCl₃) δ 8.34 (s, 1H), 7.40-7.31 (m, 2H), 7.14-7.09 (m, 1H), 7.08-6.95 (m, 6H), 6.26 (s, 1H), 4.70 (s, 2H).

5. Synthesis of compound int. 29. To a solution of [5-bromo-3-fluoro-4-(4-phenoxyanilino)-2-pyridyl]methanol (200 mg, 0.47 mmol) in NMP (20 mL) were added Zn(CN)₂ (164 mg, 1.40 mmol) and Pd(PPh₃)₄ (54 mg, 0.047 mmol). The mixture was stirred at 130° C. for 5 h under N₂ atmosphere. Water (50 mL) was added and extracted with ethyl acetate (30 mL×2). The combined organic phases were washed with brine (30 mL), dried over anhydrous Na₂SO₄, filtered and concentrated. The residue was purified by Prep-HPLC to yield title compound (55 mg). ESI-MS (EI⁺, m/z): 336.1 [M+H]⁺. ¹H NMR (400 MHz, CDCl₃) b 8.39 (s, 1H), 7.41-7.34 (m, 2H), 7.24 (d, J=2.4 Hz, 1H), 7.19-7.11 (m, 2H), 7.10-7.04 (m, 4H), 4.89 (d, J=2.4 Hz, 2H).

### Example 30. Preparation of Compound Int. 30

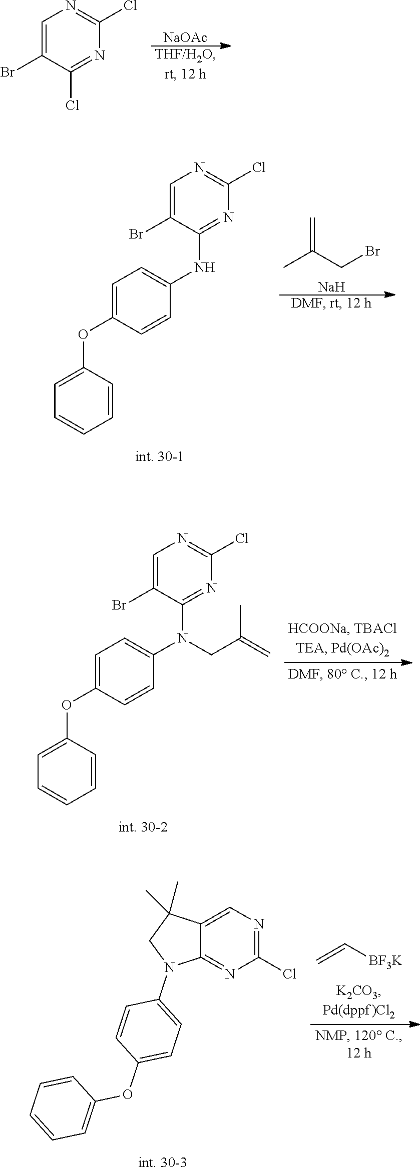

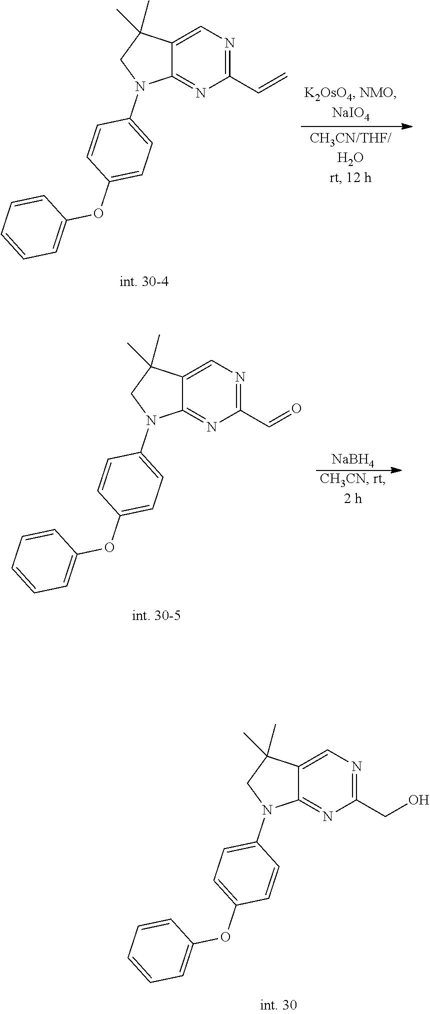 [SMILES: C=Cc1ncc2c(n1)N(c1ccc(Oc3ccccc3)cc1)CC2(C)C ; CC1(C)CN(c2ccc(Oc3ccccc3)cc2)c2nc(C=O)ncc21 ; "CH3CN, rt,2 h" ; "NaBH4" ; CC1(C)CN(c2ccc(Oc3ccccc3)cc2)c2nc(CO)ncc21]

1. Synthesis of compound int. 30-1. To a solution of 5-bromo-2,4-dichloropyrimidine (20.0 g, 87.8 mmol) in THF (200 mL)/H₂O (100 mL) was added 4-phenoxyaniline (16.4 g, 88.7 mmol) and NaOAc (21.6 g, 263 mmol) at room temperature under N₂ atmosphere, then the reaction was stirred at room temperature for 12 h. H₂O (150 mL) was added, and the mixture extracted with EA (100 mL×3). The organic layers were combined, washed with water (100 mL), and the organic phase dried over Na₂SO₄, filtered and concentrated to afford crude product. The crude was purified by column chromatography to afford title compound (23.0 g). ESI-MS (EI+, m/z): 375.99/377.95 [M+H]⁺. ¹H NMR (400 MHz, DMSO-d(₆) δ 9.30 (s, 1H), 8.44 (s, 1H), 7.57-7.50 (m, 2H), 7.40 (dd, J=8.5, 7.2 Hz, 2H), 7.21-7.10 (m, 1H), 7.08-7.00 (m, 4H).

2. Synthesis of compound int. 30-2. To a solution of 5-bromo-2-chloro-N-(4-phenoxyphenyl)pyrimidin-4-amine (10.0 g, 26.6 mmol) in DMF (50 mL) was added NaH (1.27 g, 31.9 mmol, 60%) at 0° C. under N₂ atmosphere. After 0.5 h, 3-bromo-2-methylprop-1-ene (4.66 g, 34.5 mmol) was added. The mixture was stirred at room temperature for 12 h. H₂O (50 mL) was added, and the mixture extracted with EA (50 mL×3). The organic layers were combined, washed with water (50 mL×6), dried by Na₂SO₄, filtered and concentrated. The crude was purified by column chromatography to afford title compound (6.10 g). ¹H NMR (400 MHz, DMSO-d₆) δ 8.40 (s, 1H), 7.46-7.36 (m, 2H), 7.31-7.21 (m, 2H), 7.15 (t, J=7.4 Hz, 1H), 7.01 (t, J=8.8 Hz, 4H), 4.84 (d, J=18.3 Hz, 2H), 4.54 (s, 2H), 1.73 (s, 3H).

3. Synthesis of compound int. 30-3. A mixture of 5-bromo-2-chloro-N-(2-methylallyl)-N-(4-phenoxyphenyl)pyrimidin-4-amine (9.0 g, 20.9 mmol), NaOOCH (1.51 g, 22.1 mmol), TBACl (5.81 g, 20.9 mmol), TEA (5.41 g, 53.5 mmol), Pd(OAc)₂ (938 mg, 4.18 mmol) in DMF (50 mL) was stirred at 80° C. for 12 h under N₂ atmosphere. H₂O (50 mL) was added and the mixture extracted with EA (50 mL×3). The organic layers were combined, washed with water (30 mL×6), and the organic phase dried by Na₂SO₄. The solvent was evaporated and purified column chromatography to afford title compound (4.30 g). ESI-MS (EI⁺, m/z): 352.00 [M+H]⁺. ¹H NMR (400 MHz, DMSO-d₆) δ 8.10 (s, 1H), 7.83-7.72 (m, 2H), 7.47-7.30 (m, 2H), 7.19-7.08 (m, 3H), 7.05-6.94 (m, 2H), 3.92 (s, 2H), 1.38 (s, 6H).

4. Synthesis of compound int. 30-4. A mixture of 2-chloro-5,5-dimethyl-7-(4-phenoxyphenyl)-6,7-dihydro-5H-pyrrolo[2,3-d]pyrimidine (4.30 g, 12.2 mmol), Pd(dppf)Cl₂ (8.18 g, 61.1 mmol), potassium trifluoro(vinyl)borate (893 mg, 1.22 mmol), K₂CO₃ (5.07 g, 36.7 mmol) in NMP (80 mL) was stirred at 120° C. for 12 h under N₂ atmosphere. H₂O (100 mL) was added to the mixture and extracted with EA (100 mL×3). The organic layers were combined, washed with water (100 mL×6), dried by Na₂SO₄. The solvent was evaporated to dryness and purified by column chromatography to afford title compound (3.01 g). ESI-MS (EI⁺, m/z): 344.20 [M+H]⁺. ¹H NMR (400 MHz, DMSO-d₆) δ 8.22 (s, 1H), 8.05-7.85 (m, 2H), 7.46-7.29 (m, 2H), 7.11 (dd, J=8.3, 6.1 Hz, 3H), 7.04-6.92 (m, 2H), 6.67 (dd, J=17.3, 10.4 Hz, 1H), 6.43 (dd, J=17.3, 2.2 Hz, 1H), 5.61 (dd, J=10.4, 2.2 Hz, 1H), 3.86 (s, 2H), 1.39 (s, 6H).

5. Synthesis of compound int. 30-5. A mixture of 5,5-dimethyl-7-(4-phenoxyphenyl)-2-vinyl-6,7-dihydro-5H-pyrrolo[2,3-d]pyrimidine (3.01 g, 8.76 mmol), K₂OsO4·2H2O (307 mg, 0.88 mmol), NMO (2.57 g, 21.9 mmol), NaIO4 (4.69 g, 21.9 mmol) in acetonitrile (100 mL)/THF (20 mL)/water (20 mL) was stirred at room temperature for 3.5 h under N₂ atmosphere. The reaction mixture was filtered and the filtrate was concentrated to give title compound (2.40 g). ESI-MS (EI⁺, m/z): 364.2 [M+19]⁺.

6. Synthesis of compound int. 30. A mixture of 5,5-dimethyl-7-(4-phenoxyphenyl)-6,7-dihydro-5H-pyrrolo[2,3-d]pyrimidine-2-carbaldehyde (468 mg, 1.35 mmol), NaBH₄ (102 mg, 2.71 mmol) in acetonitrile (10 mL) was stirred at 25° C. for 2 h under N₂ atmosphere. The reaction mixture was filtered. The filtrate was concentrated and purified by column chromatography to give title compound (560 mg). ESI-MS (EI+, m/z): 348.05 [M+H]⁺. ¹H NMR (400 MHz, DMSO-d₆) δ 8.20 (s, 1H), 8.02-7.84 (m, 2H), 7.37 (dd, J=8.6, 7.3 Hz, 2H), 7.14-7.05 (m, 3H), 7.01-6.93 (m, 2H), 4.41 (s, 2H), 3.86 (s, 2H), 3.33 (s, 1H), 1.38 (s, 6H).

### Example 31. Preparation of Compound Int. 31

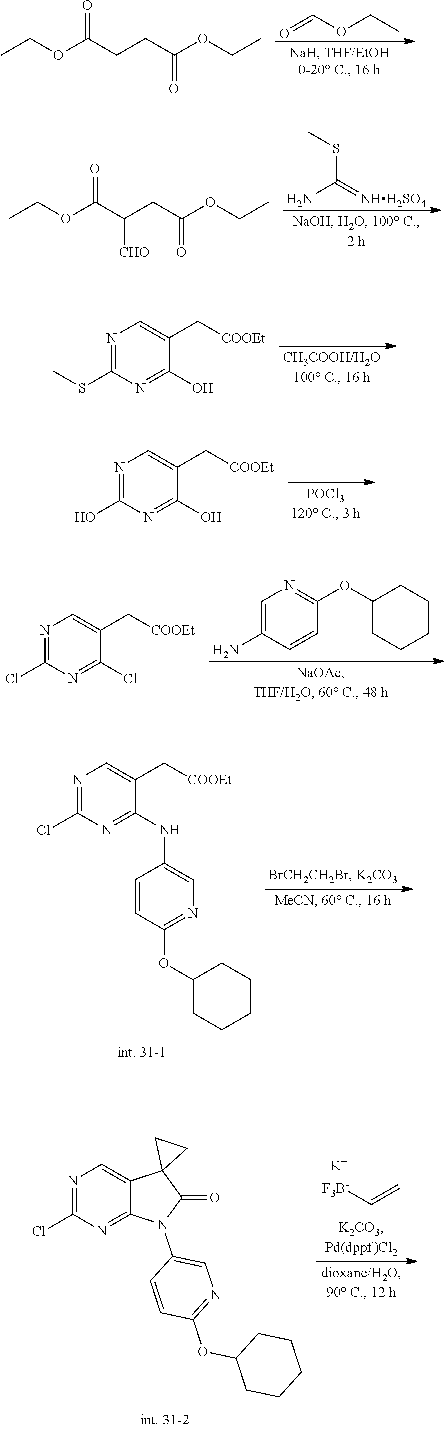

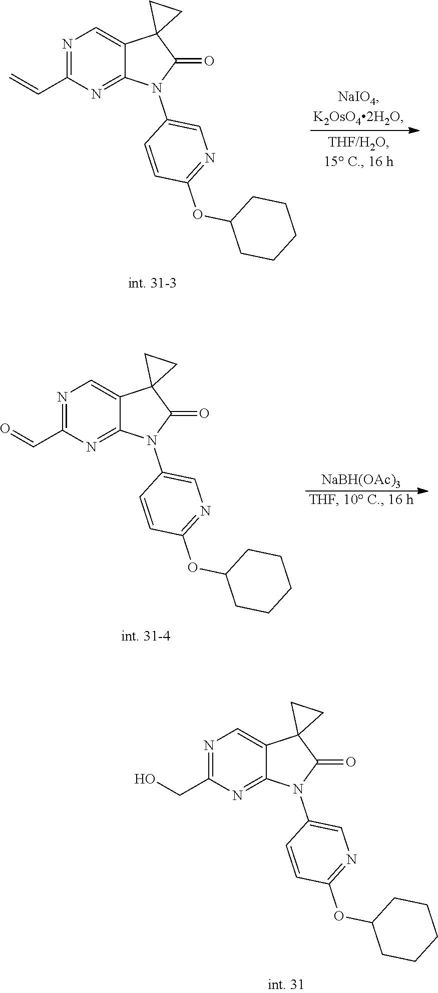 [SMILES: C=Cc1ncc2c(n1)N(c1ccc(OC3CCCCC3)nc1)C(=O)C21CC1 ; O=Cc1ncc2c(n1)N(c1ccc(OC3CCCCC3)nc1)C(=O)C21CC1 ; "THF, 10° C., 16 h" ; O=C1N(c2ccc(OC3CCCCC3)nc2)c2nc(CO)ncc2C12CC2 ; "NaBH(OAc)3"]

1. Synthesis of compound diethyl 2-formylsuccinate. To a mixture of NaH (2.30 g, 57.4 mmol, 60% in mineral) in THE (38 mL) was added the solution of diethyl succinate (10.0 g, 57.4 mmol) and ethyl formate (5.10 g, 68.9 mmol) in EtOH (10 mL) at 0° C. under N₂ atmosphere. The mixture was stirred at 20° C. for 16 h. H₂O (15 mL) was added to the mixture and the layer was separated. The aqueous layer was neutralized with 2M aqueous HCl and extracted with DCM (30 mL×3). The combined organic layers were washed with brine (30 mL), dried with Na₂SO₄, filtered, and concentrated under vacuum at room temperature to give desired product (10.0 g crude).

2. Synthesis of compound ethyl 2-(4-hydroxy-2-(methylthio)pyrimidin-5-yl)acetate. To a solution of diethyl 2-formylbutanedioate (6.7 g, 33.1 mmol) and 2-methylisothiourea (6.24 g, 33.1 mmol, H₂SO₄ salt) in H₂O (32 mL) was added the mixture of NaOH (1.99 g, 49.7 mmol) in H₂O (8 mL). The mixture was stirred at 100° C. for 2 h, then cooled down to room temperature and adjust pH=4 with 2M aqueous HCl. The mixture was filtered, filtered cake was washed with H₂O (15 mL×3). The residue was dried under vacuum to give title compound (2.0 g). ESI-MS (EI⁺, m/z): 229.0 [M+H]⁺.

3. Synthesis of compound ethyl 2-(2,4-dihydroxypyrimidin-5-yl)acetate. A mixture of ethyl 2-(4-hydroxy-2-methylsulfanyl-pyrimidin-5-yl)acetate (1.0 g, 4.38 mmol) in CH₃COOH (4 mL, 4.38 mmol) and H₂O (4 mL) stirred at 100° C. for 16 h. The mixture was concentrated under vacuum to give title compound (750 mg). ESI-MS (EI⁺, m/z): 198.2 [M+H]⁺.

4. Synthesis of compound ethyl 2-(2,4-dichloropyrimidin-5-yl)acetate. A mixture of ethyl 2-(2,4-dihydroxypyrimidin-5-yl)acetate (400 mg, 2.02 mmol) in POCl₃ (5 mL, 53.6 mmol) was stirred at 120° C. for 3 h. The mixture was concentrated under vacuum. The residue was purified by silica gel chromatography to give title compound (188 mg). ESI-MS (EI⁺, m/z): 235.0 [M+H]⁺.

5. Synthesis of compound int. 31-1. To a solution of ethyl 2-(2,4-dichloropyrimidin-5-yl)acetate (188 mg, 0.80 mmol) in THF (2 mL) and water (2 mL) was added 6-(cyclohexoxy)pyridin-3-amine (154 mg, 0.80 mmol) and NaOAc (131 mg, 1.60 mmol). The mixture was stirred at 60° C. for 48 h. The reaction mixture was brown solution. The mixture was quenched with sat. aqueous NH₄Cl (30 mL) and extracted with EtOAc (30 mL×3). The organic layer was washed with brine (50 mL), dried over Na₂SO₄, filtered and concentrated. The crude product was purified by silica gel chromatography to give title compound (250 mg).

6. Synthesis of compound int. 31-2. A solution of ethyl 2-(2-chloro-4-((6-(cyclohexyloxy)pyridin-3-yl)amino)pyrimidin-5-yl)acetate (880 mg, 2.25 mmol) in MeCN (20 mL) was added K₂CO₃ (1.56 g, 11.26 mmol) and 1,2-dibromoethane (846 mg, 4.50 mmol) under N₂. The mixture was stirred at 60° C. for 16 h. The mixture was quenched with saturated aqueous NH₄Cl (15 mL) and extracted with EtOAc (10 mL×3). The organic layer was washed with brine (50 mL), dried over Na₂SO₄, filtered and concentrated. The residue was purified by silica gel chromatography to give title compound (430 mg). ESI-MS (EI⁺, m/z): 371.1 [M+H]⁺.

7. Synthesis of compound int. 31-3. To a solution of 2′-chloro-7′-(6-(cyclohexyloxy)pyridin-3-yl)spiro[cyclopropane-1,5′-pyrrolo[2,3-d]pyrimidin]-6′(7′H)-one (458 mg, 1.24 mmol) and potassium trifluoro(vinyl)borate (199 mg, 1.48 mmol) in dioxane (14 mL) and H₂O (7 mL) was added K₂CO₃ (427 mg, 3.09 mmol) and Pd(dppf)Cl₂ (90 mg, 0.12 mmol). The reaction mixture was stirred at 90° C. for 12 h under N₂ atmosphere. The reaction mixture was quenched by saturated aqueous NH₄Cl (20 mL) and extracted with EA (25 mL×3). The combined organic phase was washed with brine (25 mL), dried over anhydrous Na₂SO₄, filtered and concentrated. The residue was purified by silica gel chromatography to give title compound (240 mg).

8. Synthesis of compound int. 31-4. To a solution of 7′-(6-(cyclohexyloxy)pyridin-3-yl)-2′-vinylspiro[cyclopropane-1,5′-pyrrolo[2,3-d]pyrimidin]-6′(7′H)-one (215 mg, 0.59 mmol) in THF (5 mL) and H₂O (1 mL) were added K₂OsO₄·2H₂O (18 mg, 0.059 mmol) and NaIO₄ (512 mg, 2.37 mmol). The mixture was stirred at 15° C. for 16 h. The mixture was quenched with H₂O (30 mL) and extracted with EtOAc (30 mL×3). The organic layer was separated, washed with brine (30 mL), dried over Na₂SO₄, filtered and concentrated under vacuum to give title compound (215 mg).

9. Synthesis of compound int. 31. To a solution of 7′-(6-(cyclohexyloxy)pyridin-3-yl)-6′-oxo-6′,7′-dihydrospiro[cyclopropane-1,5′-pyrrolo[2,3-d]pyrimidine]-2′-carbaldehyde (215 mg, 0.59 mmol) in THF (1 mL) was added NaBH(OAc)₃ (250 mg, 1.18 mmol) and the mixture was stirred at 10° C. for 16 h. The mixture was filtered and concentrated. The residue was purified by silica gel chromatography to give title compound (130 mg). ESI-MS (EI⁺, m/z): 398.2 [M+Na]*.

### Example 32. Preparation of Compound Int. 32

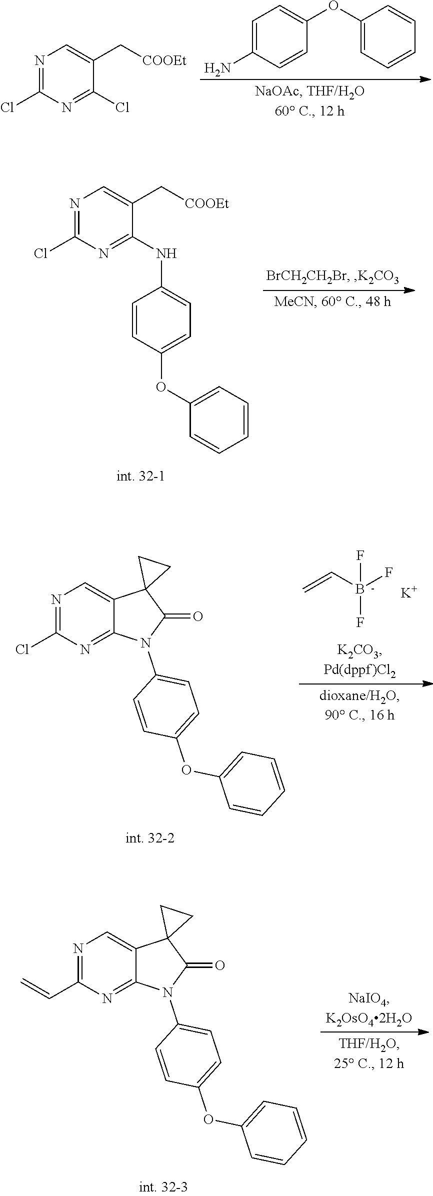

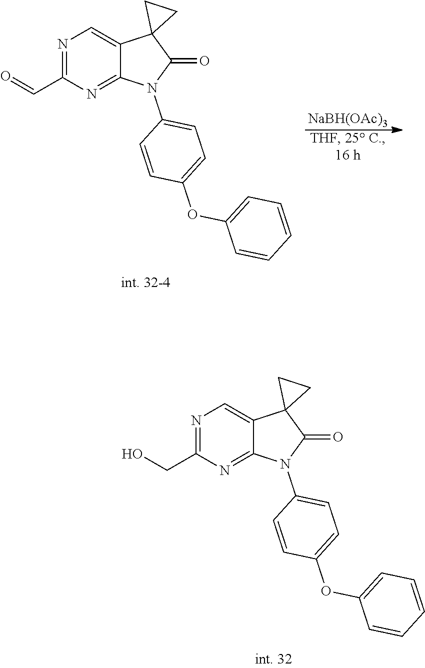 [SMILES: O=Cc1ncc2c(n1)N(c1ccc(Oc3ccccc3)cc1)C(=O)C21CC1 ; "THF, 25° C.,16 h" ; O=C1N(c2ccc(Oc3ccccc3)cc2)c2nc(CO)ncc2C12CC2 ; "NaBH(OAc)3"]

1. Synthesis of compound int. 32-1. To a solution of ethyl 2-(2,4-dichloropyrimidin-5-yl)acetate (1.5 g, 6.38 mmol) in THF (20 mL) and water (10 mL) was added 4-phenoxyaniline (1.42 g, 7.66 mmol) and NaOAc (1.31 g, 15.9 mmol) under N₂ atmosphere. The mixture was stirred at 60° C. for 12 h. Water (50 mL) was added and extracted with EtOAc (50 mL×2). The combined organic phases were washed with brine (20 mL), dried over anhydrous Na₂SO₄, filtered and concentrated to give a residue. The residue was purified by silica gel chromatography to give title compound (1.0 g). ¹H NMR (400 MHz, CDCl₃) δ 8.35 (s, 1H), 8.02 (s, 1H), 7.60-7.54 (m, 2H), 7.34 (m, 2H), 7.13-7.08 (m, 1H), 7.06-6.98 (m, 4H), 4.23 (q, J=7.2 Hz, 2H), 3.53 (s, 2H), 1.31 (t, J=7.2 Hz, 3H).

2. Synthesis of compound int. 32-2. To a solution of ethyl 2-(2-chloro-4-((4-phenoxyphenyl)amino)pyrimidin-5-yl)acetate (1.25 g, 3.26 mmol) in MeCN (30 mL) was added 1,2-dibromoethane (1.22 g, 6.51 mmol) and K₂CO₃ (2.25 g, 16.3 mmol) under N₂ atmosphere. The mixture was stirred at 60° C. for 48 h. Water (40 mL) was added and extracted with EtOAc (50 mL×2). The combined organic phases were washed with brine (20 mL), dried over anhydrous Na₂SO₄, filtered and concentrated to give a residue. The residue was purified by silica gel chromatography to give title compound (0.25 g). ESI-MS (EI⁺, m/z): 364.1 [M+H]⁺.

3. Synthesis of compound int. 32-3. To a mixture of 2′-chloro-7′-(4-phenoxyphenyl)spiro[cyclopropane-1,5′-pyrrolo[2,3-d]pyrimidin]-6′(7′H)-one (0.23 g, 0.63 mmol) in dioxane (5 mL) and H₂O (2 mL) was added potassium trifluoro(vinyl)borate (127 mg, 0.95 mmol) and K₂CO₃ (218 mg, 1.58 mmol), then Pd(dppf)Cl₂ (93 mg, 0.13 mmol) was added. The mixture was stirred at 90° C. for 16 h under N₂ atmosphere. The reaction mixture was quenched by saturated aqueous NH₄Cl (15 mL) and extracted with EtOAc (20 mL×3). The combined organic phase was washed with brine (10 mL), dried over anhydrous Na₂SO₄, filtered and concentrated. The residue was purified by silica gel chromatography to give title compound (0.12 g). ESI-MS (EI⁺, m/z): 356.1 [M+H]⁺.

4. Synthesis of compound int. 32-4. To a solution of 7′-(4-phenoxyphenyl)-2′-vinylspiro[cyclopropane-1,5′-pyrrolo[2,3-d]pyrimidin]-6′(7′H)-one (0.12 g, 0.34 mmol) in THF (4 mL) and H₂O (0.8 mL) was added NaIO₄ (182 mg, 0.84 mmol) and K₂OsO₄·2H₂O (11 mg, 0.033 mmol). The reaction mixture was stirred at 25° C. for 12 h. The reaction mixture was filtered. The filtrate was diluted with water (20 mL) and extracted with EtOAc (20 mL×2). The combined organic phases were washed with brine (10 mL), dried over anhydrous Na₂SO₄, filtered and concentrated to give title compound (0.12 g crude).

5. Synthesis of compound int. 32. To a solution of 6′-oxo-7′-(4-phenoxyphenyl)-6′,7′-dihydrospiro[cyclopropane-1,5′-pyrrolo[2,3-d]pyrimidine]-2′-carbaldehyde (0.12 g crude) in THF (4 mL) was added NaBH(OAc)₃ (214 mg, 1.01 mmol) at 25° C. The reaction mixture was stirred at 25° C. for 6 h, quenched by saturated aqueous NH₄Cl (15 mL) and extracted with EtAOc (25 mL×3). The combined organic phase was washed with brine (10 mL), dried over anhydrous Na₂SO₄, filtered and concentrated. The residue was purified by silica gel chromatography to give title compound (30 mg).

### Example 33. Preparation of Compound A1

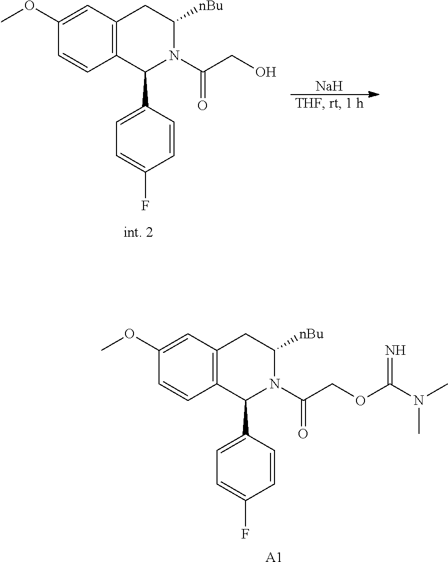

A solution of 1-((1S,3S)-3-butyl-1-(4-fluorophenyl)-6-methoxy-3,4-dihydroisoquinolin-2(1H)-yl)-2-Hydroxyethan-1-one (200 mg, 0.54 mmol) in 40 mL of THF was added NaH (51.2 mg, 2.16 mmol) and stirred for 30 min at room temperature. The solvent was removed under reduced pressure, then added N,N-dimethylcyanamide (1 mL) and stirred for 1 h. The mixture was purified by prep-HPLC to afford the title compound (25 mg). ¹H NMR (400 MHz, Chloroform-d) δ 8.35 (s, 1H), 7.16 (dd, J=8.4, 5.4 Hz, 2H), 6.97 (q, J=8.6 Hz, 2H), 6.89-6.60 (m, 3H), 6.15-5.76 (m, 1H), 5.36 (s, 1H), 4.59 (s, 2H), 4.40 (s, 4H), 3.79 (d, J=9.1 Hz, 3H), 3.22 (s, 2H), 3.11-2.90 (m, 6H), 2.72 (dd, J=16.7, 10.7 Hz, 1H), 1.53 (dq, J=13.9, 7.1 Hz, 1H), 0.99 (s, 1H), 0.84 (d, J=7.3 Hz, 3H).

### Example 34. Preparation of Compound A2

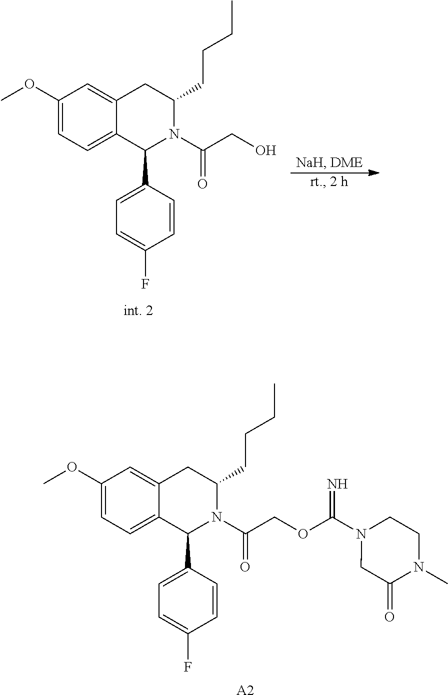

To a solution of 1-[(1S,3S)-3-butyl-1-(4-fluorophenyl)-6-methoxy-1,2,3,4-tetrahydroisoquinolin-2-yl]-2-hydroxyethan-1-one (100 mg, 0.27 mmol) in DME (1 mL) was added NaH (7.78 mg, 0.32 mmol, 60%). After stirring at room temperature for 1 h, 4-methyl-3-oxopiperazine-1-carbonitrile (37.6 mg, 0.27 mmol) was added into solution and the mixture was stirred for 1 h. The reaction was purified by Prep-HPLC to give title compound (12 mg). ESI-MS (EI+, m/z): 511.40.

### Example 35. Preparation of Compound A3

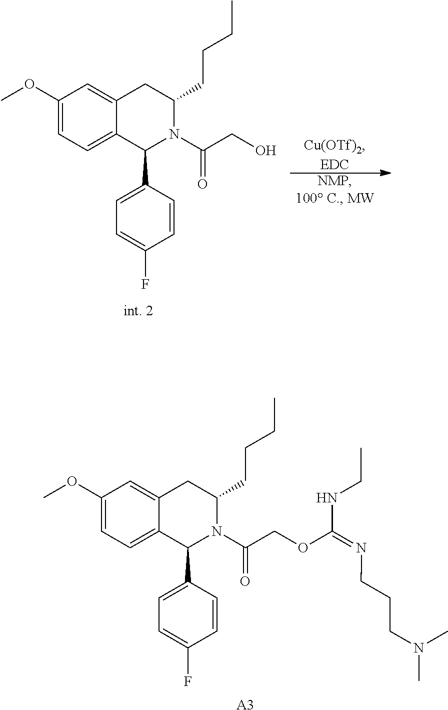

A solution of 1-[(1S,3S)-3-butyl-1-(4-fluorophenyl)-6-methoxy-1,2,3,4-tetrahydroisoquinolin-2-yl]-2-hydroxyethan-1-one (50 mg, 0.13 mmol), EDC (25.1 mg, 0.16 mmol) and bis(((trifluoromethyl)sulfonyl)oxy)copper (1.43 mg, 0.01 mmol) in NMP (1 mL) was stirred at 100° C. in a sealed tube for 5 min. The solution was purified by Prep-HPLC to get title compound (20 mg). ESI-MS (EI⁺, m/z): 527.25. ¹H NMR (400 MHz, DMSO-d₆) δ 7.48 (s, 1H), 7.31 (s, 2H), 7.17-6.93 (m, 2H), 6.81 (d, J=9.6 Hz, 2H), 6.07 (d, J=11.4 Hz, 1H), 4.99 (d, J=14.7 Hz, 1H), 3.71 (s, 3H), 2.86 (s, 2H), 2.36 (t, J=7.1 Hz, 3H), 2.20 (s, 6H), 1.55 (d, J=44.8 Hz, 4H), 1.23 (s, 6H), 1.05 (s, 3H), 0.82 (s, 3H).

### Example 36. Preparation of Compound A4

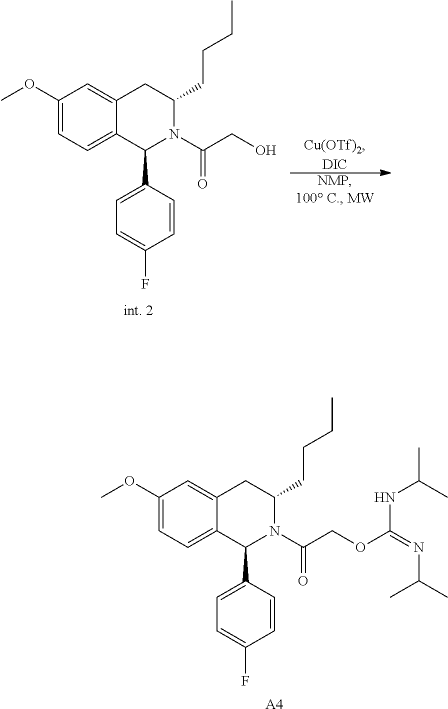

Procedure as compound A3. ESI-MS (EI⁺, m/z): 498.40. ¹H NMR (400 MHz, DMSO-d₆) δ 7.48 (d, J=7.8 Hz, 1H), 7.30 (s, 2H), 7.17-6.91 (m, 2H), 6.81 (d, J=11.0 Hz, 2H), 6.13 (d, J=49.4 Hz, 1H), 4.93 (dd, J=31.2, 17.2 Hz, 1H), 4.48 (d, J=68.9 Hz, 1H), 4.08-3.87 (m, 1H), 3.71 (s, 4H), 3.63 (d, J=6.9 Hz, 1H), 2.96-2.67 (m, 3H), 1.56 (d, J=45.1 Hz, 1H), 1.36-0.70 (m, 22H).

### Example 37. Preparation of Compound B1

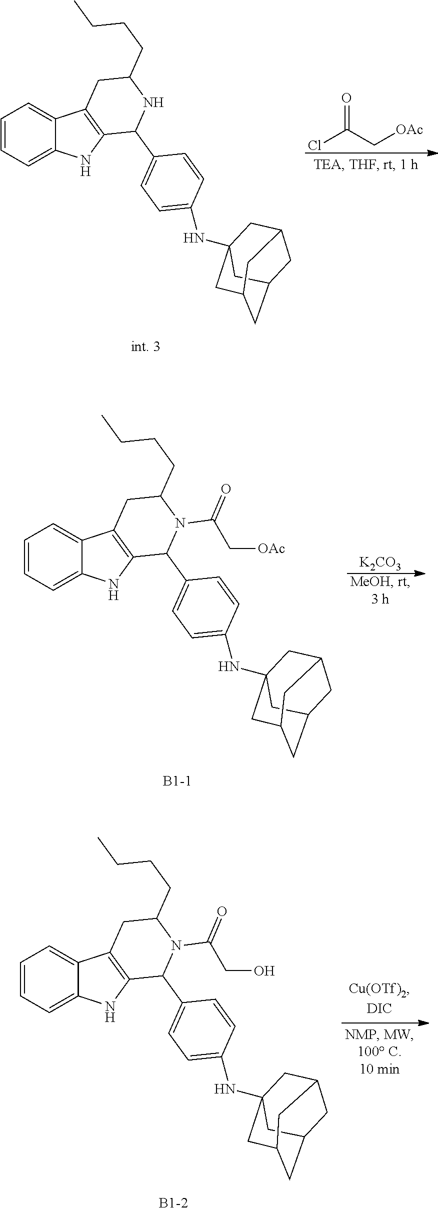 [SMILES: CCCCC1Cc2c([nH]c3ccccc23)C(c2ccc(NC34CC5CC(CC(C5)C3)C4)cc2)N1 ; CC(=O)OCC(=O)Cl ; CCCCC1Cc2c([nH]c3ccccc23)C(c2ccc(NC34CC5CC(CC(C5)C3)C4)cc2)N1C(=O)COC(C)=O ; "K2CO3" ; "MeOH, rt,3 h" ; CCCCC1Cc2c([nH]c3ccccc23)C(c2ccc(NC34CC5CC(CC(C5)C3)C4)cc2)N1C(=O)CO]

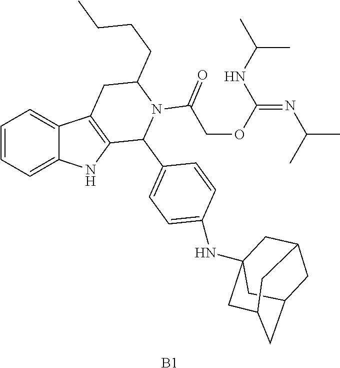 [SMILES: CCCCC1Cc2c([nH]c3ccccc23)C(c2ccc(NC34CC5CC(CC(C5)C3)C4)cc2)N1C(=O)CO/C(=N\C(C)C)NC(C)C ; "B1"]

1. Synthesis of compound B1-1. To a solution of N—{4-[(1S,3S)-3-butyl-1H, 2H, 3H, 4H, 9H-pyrido[3,4-b]indol-1-yl]phenyl}adamantan-1-amine (100 mg, 0.22 mmol) and TEA (44.5 mg, 0.44 mmol) in THF (1 mL) was added 2-chloro-2-oxoethyl acetate (27.0 mg, 0.2 mmol) dropwise at 0° C. The resulting solution was stirred at room temperature for 1 h. The mixture was diluted with EA (10 mL) and was washed with brine (2×10 mL). The organic layer was dried and concentrated to get title compound (105 mg). ESI-MS (EI+, m/z): 554.30.

2. Synthesis of compound B1-2. A solution of 2-((1S,3S)-1-(4-(((1S,3R,5S)-adamantan-1-yl)amino)phenyl)-3-butyl-1,3,4,9-tetrahydro-2H-pyrido[3,4-b]indol-2-yl)-2-oxoethyl acetate (105 mg, 0.19 mmol) and K₂CO₃ (78.8 mg, 0.57 mmol) in MeOH (2 mL) was stirred at room temperature for 3 h. The reaction mixture was filtered and the filtrate was concentrated to give the crude title compound (90 mg). ESI-MS (EI⁺, m/z): 512.25.

3. Synthesis of compound B1. A mixture of 1-((1S,3S)-1-(4-(((1S,3R,5S)-adamantan-1-yl)amino)phenyl)-3-butyl-1,3,4,9-tetrahydro-2H-pyrido[3,4-b]indol-2-yl)-2-hydroxyethan-1-one (50 mg, 0.1 mmol), DIC (13.6 mg, 0.11 mmol) and Cu(OT_{f})₂ (2.13 mg, 0.01 mmol) in NMP (1 mL) was stirred at 100° C. in microwave for 10 min. The reaction was purified by Prep-HPLC to give title compound (18 mg). ESI-MS (EI⁺, m/z): 638.50. ¹H NMR (400 MHz, DMSO-d(₆) δ 11.02 (s, 1H), 8.26 (s, 1H), 7.44 (d, J=7.8 Hz, 1H), 7.31-7.27 (m, 11H), 7.07 (d, J=7.7 Hz, 1H), 7.00 (d, J=8.0 Hz, 3H), 6.83 (s, 1H), 6.66 (d, J=8.3 Hz, 2H), 5.16 (d, J=14.7 Hz, 11H), 4.95 (d, J=14.8 Hz, 11H), 4.18-3.97 (m, 2H), 3.07 (dd, J=15.7, 6.6 Hz, 2H), 2.79-2.66 (m, 2H), 2.02 (t, J=3.5 Hz, 4H), 1.81 (d, J=2.8 Hz, 7H), 1.61 (d, J=3.4 Hz, 8H), 1.23 (s, 5H), 1.00 (d, J=6.5 Hz, 5H), 0.91-0.72 (m, 3H), 0.65 (q, J=7.8, 6.9 Hz, 1H), 0.56 (t, J=7.0 Hz, 3H).

### Example 38. Preparation of Compound B2

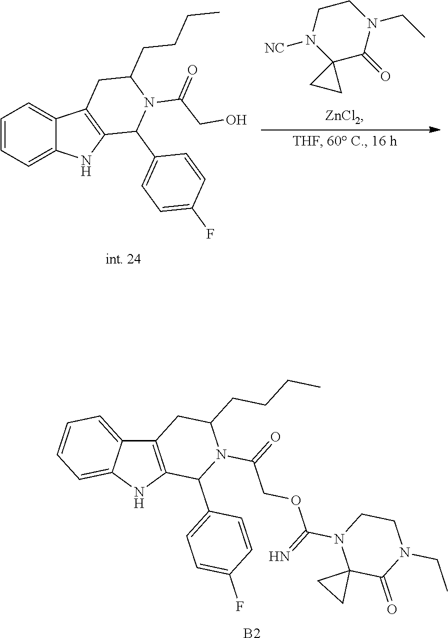

To a solution of 1-[(1S,3S)-3-butyl-1-(4-fluorophenyl)-1,3,4,9-tetrahydropyrido[3,4-b]indol-2-yl]-2-hydroxy-ethanone (100 mg, 0.26 mmol) and 7-ethyl-8-oxo-4,7-diazaspiro[2.5]octane-4-carbonitrile (141 mg, 0.79 mmol) in THF (10 mL) was added ZnCl₂ (143 mg, 1.05 mmol), the mixture was stirred at 60° C. for 16 h under N₂ atmosphere. The solvent was removed to yield a residue which was purified by Prep-HPLC to give title compound (26 mg). ESI-MS (EI⁺, m/z): 560.3 [M+H]⁺. ¹H NMR (400 MHz, CDCl₃) δ 8.33 (s, 1H), 7.54-7.41 (m, 3H), 7.35 (dt, J=8.8, 3.2 Hz, 2H), 7.25-7.18 (m, 1H), 7.17-7.08 (m, 2H), 7.07-6.98 (m, 2H), 6.93 (dd, J=9.6, 7.6 Hz, 2H), 5.16-4.83 (m, 2H), 4.62 (s, 1H), 4.12 (s, 1H), 4.03 (d, J=7.2 Hz, 1H), 3.88 (t, J=5.6 Hz, 2H), 3.60-3.56 (m, 2H), 3.46-3.44 (m, 2H), 3.37-3.33 (m, 1H), 2.95-2.69 (m, 2H), 1.79-1.62 (m, 3H), 1.53-1.30 (m, 4H), 1.16-1.12 (m, 4H), 1.09-0.97 (m, 3H), 0.96-0.93 (m, 2H), 0.81-0.66 (m, 2H), 0.58 (t, J=7.2 Hz, 3H).

### Example 39. Preparation of Compound C₁

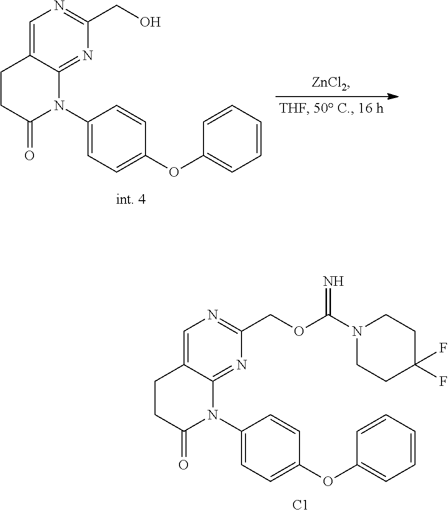

To a solution of 2-(hydroxymethyl)-8-(4-phenoxyphenyl)-5,8-dihydropyrido[2,3-d]pyrimidin-7(6H)-one (50 mg, 0.14 mmol) and 4,4-difluoropiperidine-1-carbonitrile (81.8 mg, 0.56 mmol) in THF (2 mL) was added zinc(II) chloride (76.3 mg, 0.56 mmol) at 25° C. under N₂ atmosphere. The reaction was stirred at 50° C. for 16 h. The reaction was concentrated. The crude was purified by TLC to give title compound (15.3 mg).

### Example 40. Preparation of Compound D1

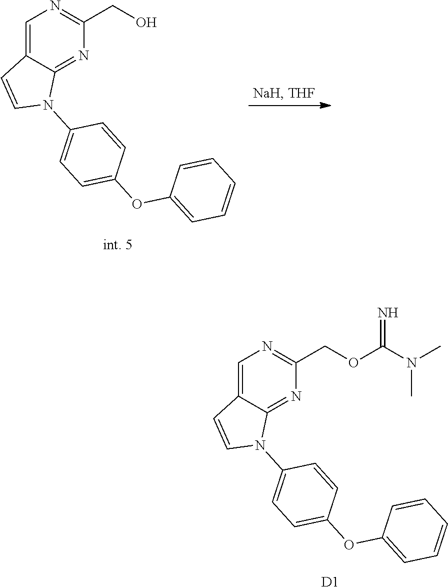

A solution of [7-(4-phenoxyphenyl)-7H-pyrrolo[2,3-d]pyrimidin-2-yl]methanol (100 mg, 0.32 mmol) in 1 mL of THF was added NaH (8.32 mg, 0.35 mmol). The mixture was stirred at room temperature for 2 h in an inert atmosphere. The mixture was concentrated. The residue was dissolved in 1 mL of N,N-dimethylcyanamide and stirred at room temperature for 2 h. The mixture was purified by Prep-HPLC to give title compound (4.5 mg). ESI-MS (EI⁺, m/z): 388.15.

### Example 41. Preparation of Compound D2 and Various Other Compounds

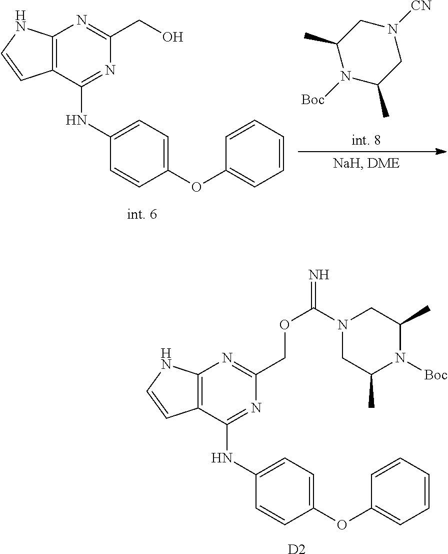

To a solution of {4-[(4-phenoxyphenyl)amino]-7H-pyrrolo[2,3-d]pyrimidin-2-yl}methanol (50 mg, 0.15 mmol) in DME (1 mL) was added NaH (9 mg, 0.23 mmol) and stirred at 25° C. for 1 h, then the solvent was removed, tert-butyl (2R,6S)-4-cyano-2,6-dimethylpiperazine-1-carboxylate (35.9 mg, 0.15 mmol) was added and stirred at 25° C. for 2 h. The solution was filtered, concentrated in vacuo, purified by Prep-HPLC to get title compound (12 mg). ESI-MS (EI⁺, m/z): 572.40. ¹H NMR (400 MHz, DMSO-d₆) δ 11.68 (s, 1H), 9.36 (s, 1H), 7.93 (d, J=8.9 Hz, 2H), 7.41-7.33 (m, 2H), 7.21 (t, J=2.9 Hz, 1H), 7.09 (t, J=7.4 Hz, 1H), 7.04-6.95 (m, 4H), 6.75 (dd, J=3.5, 1.9 Hz, 1H), 5.13 (s, 2H), 4.01-3.94 (m, 2H), 3.86 (d, J=13.1 Hz, 2H), 2.88 (dd, J=13.2, 4.5 Hz, 2H), 1.40 (s, 9H), 1.10 (d, J=6.7 Hz, 6H).

Additional compounds were synthesized as compound D2, for example:

| Compound | Structure | LCMS, NMR |
|---|---|---|
| D3 | 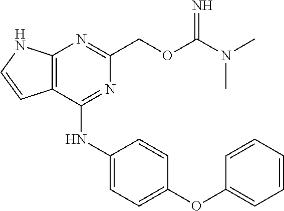 | ESI-MS (EI+, m/z): 352.20 ¹H NMR (400 MHz, DMSO-d₆) δ 11.75 (s, 1H), 9.41 (s, 1H), 8.28 (s, 1H), 7.91 (d, J = 8.8 Hz, 2H), 7.38 (dd, J = 8.6, 7.2 Hz, 2H), 7.22 (t, J = 2.8 Hz, 1H), 7.11 (t, J = 7.4 Hz, 1H), 7.00 (dd, J = 9.0, 7.3 Hz, 4H), 6.76 (d, J = 2.9 Hz, 1H), 5.22 (d, J = 5.7 Hz, 2H), 2.91 (d, J = 3.0 Hz, 6H). |
| D4 | 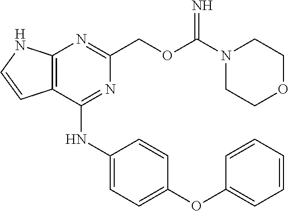 [SMILES: N=C(OCc1nc(Nc2ccc(Oc3ccccc3)cc2)c2cc[nH]c2n1)N1CCOCC1] | ESI-MS (EI+, m/z): 443.35. ¹H NMR (400 MHz, DMSO-d₆) δ11.72 (s, 1H), 9.39 (s, 1H), 8.24 (s, 1H), 7.95-7.90 (m, 2H), 7.41-7.34 (m, 2H), 7.22 (dd, J = 3.5, 2.3 Hz, 1H), 7.14-7.07 (m, 1H), 7.04-6.97 (m, 4H), 6.75 (dd, J = 3.5, 1.9 Hz, 1H), 5.15 (s, 2H), 3.55 (t, J = 4.8 Hz, 4H), 3.30 (t, J = 4.9 Hz, 4H). |
| D5 | 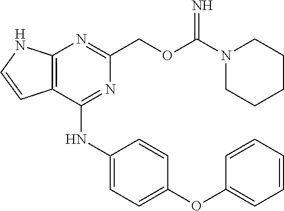 [SMILES: N=C(OCc1nc(Nc2ccc(Oc3ccccc3)cc2)c2cc[nH]c2n1)N1CCCCC1] | ESI-MS (EI+, m/z): 443.35. ¹H NMR (400 MHz, DMSO-d₆) δ 11.64 (s, 1H), 9.32 (s, 1H), 8.27 (s, 1H), 7.92 (d, J = 8.8 Hz, 2H), 7.38 (t, J = 7.8 Hz, 2H), 7.20 (d, J = 3.4 Hz, 1H), 7.11 (t, J = 7.3 Hz, 1H), 7.00 (dd, J = 8.7, 3.6 Hz, 4H), 6.75 (d, J = 3.4 Hz, 1H), 5.18 (s, 2H), 3.35 (t, J = 5.2 Hz, 4H), 1.53-1.43 (m, 6H). |
| D6 | 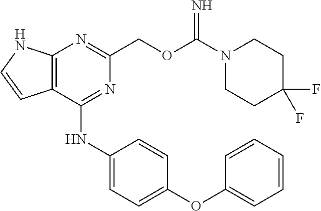 [SMILES: N=C(OCc1nc(Nc2ccc(Oc3ccccc3)cc2)c2cc[nH]c2n1)N1CCC(F)(F)CC1] | ESI-MS (EI+, m/z): 479.25. ¹H NMR (400 MHz, DMSO-d₆) δ 11.71 (s, 1H), 9.39 (s, 1H), 7.92 (d, J = 9.0 Hz, 2H), 7.39-7.35 (m, 2H), 7.24-7.20 (m, 1H), 7.10 (t, J = 7.4 Hz, 1H), 7.07- 6.96 (m, 5H), 6.75 (dd, J = 3.3, 1.8 Hz, 1H), 5.14 (s, 2H), 3.48 (s, 4H), 1.91 (dt, J = 14.2, 8.3 Hz, 4H). |
| D7 | 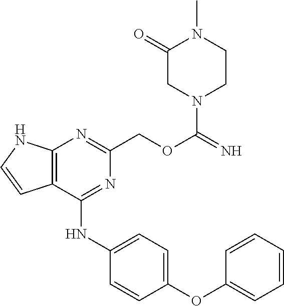 [SMILES: CN1CCN(C(=N)OCc2nc(Nc3ccc(Oc4ccccc4)cc3)c3cc[nH]c3n2)CC1=O] | ESI-MS(EI⁺, m/z) : 471.2. ¹H NMR (400 MHz, DMSO-d₆): δ 11.71 (s, 1H), 9.38 (s, 1H), 8.24 (s, 1H), 7.94 (d, J = 8.8 Hz, 2H), 7.37 (t, J = 7.9 Hz, 2H), 7.21 (t, J = 2.9 Hz, 1H), 7.10 (t, J = 7.4 Hz, 1H), 7.00 (t, J = 8.8 Hz, 4H), 6.75 (s, 1H), 5.11 (s, 2H), 3.92 (s, 2H), 3.60-3.58 (m, 2H), 3.29- 3.27 (m, 2H), 2.80 (s, 3H). |
| D8 | 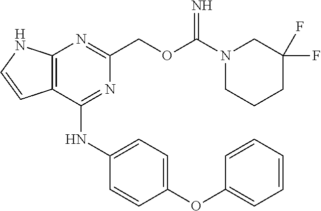 [SMILES: N=C(OCc1nc(Nc2ccc(Oc3ccccc3)cc2)c2cc[nH]c2n1)N1CCCC(F)(F)C1] | ¹H NMR (400 MHz, DMSO- d₆) δ 11.72 (s, 1H), 9.37 (s, 1H), 8.20 (s, 1H), 7.95 (d, J = 9.0 Hz, 2H), 7.37 (t, J = 8.0 Hz, 2H), 7.25-7.20 (m, 1H), 7.10 (t, J = 7.4 Hz, 1H), 7.00 (dd, J = 8.3, 4.4 Hz, 4H), 6.76 (s, 1H), 5.07 (s, 2H), 3.65 (d, J = 12.0 Hz, 2H), 3.37 (d, J = 4.7 Hz, 2H), 1.97 (dt, J = 14.3, 7.3 Hz, 2H), 1.63 (d, J = 5.9 Hz, 2H). |

### Example 42. Preparation of Compound D9

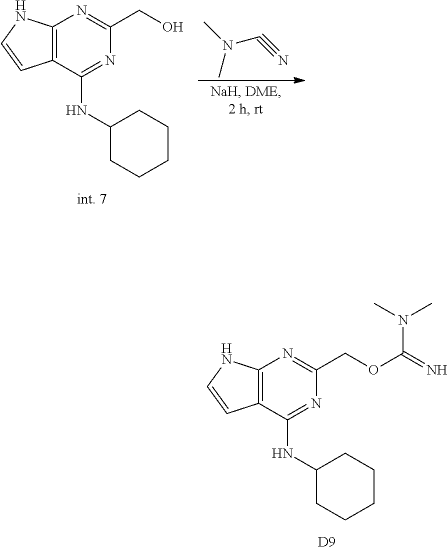

To a solution of [4-(cyclohexylamino)-7H-pyrrolo[2,3-d]pyrimidin-2-yl]methanol (50 mg, 0.2 mmol) in DME (2 mL) was added NaH (7.2 mg, 0.18 mmol, 60%). The mixture was stirred at room temperature for 1 h, then the solvent was evaporated to dryness. The residue was dissolved in N,N-dimethylcyanamide (0.5 mL) and stirred at room temperature for 1 h. The mixture was filtrated and the filtration was purified by Prep-HPLC to get the desired product (2.1 mg). ESI-MS (EI⁺, m/z): 317.30. ¹H NMR (400 MHz, Chloroform-d) δ 11.30 (s, 1H), 8.66 (s, 1H), 7.11 (d, J=3.2 Hz, 1H), 6.34 (s, 1H), 5.30 (s, 2H), 4.01 (s, 1H), 3.13 (s, 6H), 2.15-2.04 (m, 2H), 1.85-1.77 (m, 2H), 1.68 (dt, J=13.1, 3.6 Hz, 1H), 1.44 (d, J=12.5 Hz, 2H), 1.35-1.24 (m, 4H).

### Example 43. Preparation of Compound D10

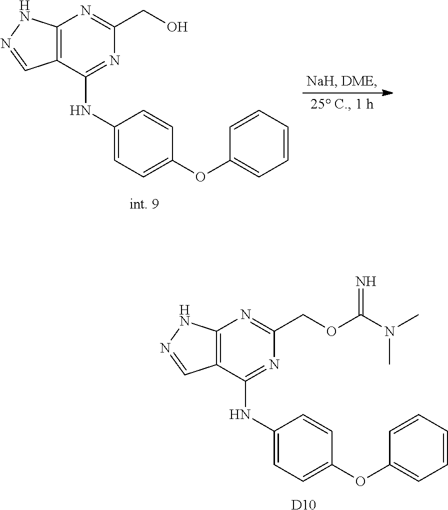

A solution of {4-[(4-phenoxyphenyl)amino]-1H-pyrazolo[3,4-d]pyrimidin-6-yl}methanol (10 mg, 0.03 mmol) in DME (0.3 mL) was added NaH (2.4 mg, 0.06 mmol, 60%) and the mixture was stirred at 25° C. for 20 min and then dimethyl-cyanamide (2.1 mg, 0.03 mmol) was added. The mixture was stirred at 25° C. for 1 h. ESI-MS (EI⁺, m/z): 404.25. ¹H NMR (400 MHz, Methanol-d₄) δ 8.54 (s, 1H), 7.69 (d, J=8.4 Hz, 2H), 7.38 (t, J=7.9 Hz, 2H), 7.13 (t, J=7.5 Hz, 1H), 7.06-7.01 (m, 4H), 5.46 (s, 2H), 3.12 (s, 6H).

### Example 44. Preparation of Compound D11

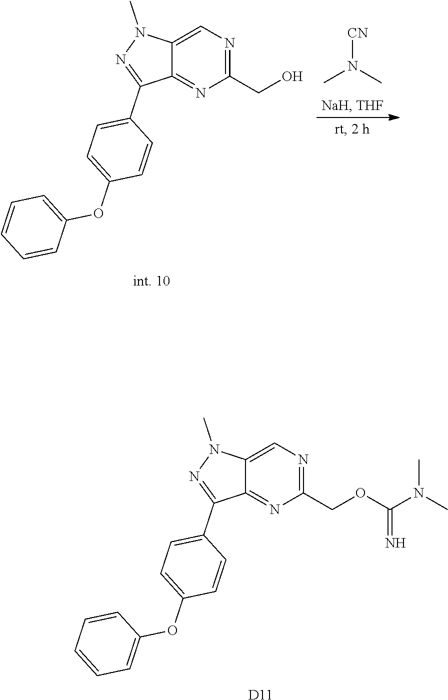

A mixture of (1-methyl-3-(4-phenoxyphenyl)-1H-pyrazolo[4,3-d]pyrimidin-5-yl)methanol (50 mg, 0.15 mmol) was added NaH (9.03 mg, 0.23 mmol, 60%) at room temperature under N₂ atmosphere. After stirred for 0.5 h, the mixture was concentrated. The residue was dissolved in N,N-dimethylcyanamide (0.5 mL) and stirred for another 1.5 h. The mixture was concentrated and the residue was purified by Prep-HPLC to afford title compound (7 mg). ESI-MS (EI⁺, m/z): 403.10. ¹H NMR (400 MHz, Chloroform-d) δ 9.14 (s, 1H), 8.40 (d, J=8.5 Hz, 2H), 7.36 (t, J=7.7 Hz, 2H), 7.12 (d, J=8.4 Hz, 3H), 7.07 (d, J=8.0 Hz, 2H), 5.72 (s, 2H), 4.22 (s, 3H), 3.12 (s, 6H).

### Example 45. Preparation of Compound D12

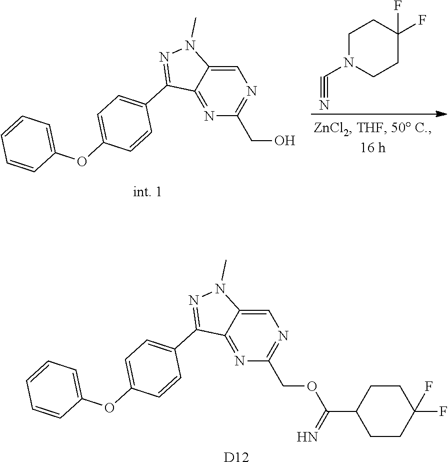 [SMILES: Cn1nc(-c2ccc(Oc3ccccc3)cc2)c2nc(CO)ncc21 ; N#CN1CCC(F)(F)CC1 ; Cn1nc(-c2ccc(Oc3ccccc3)cc2)c2nc(COC(=N)C3CCC(F)(F)CC3)ncc21 ; "D12"]

Synthesis of compound D12. To a solution of (1-methyl-3-(4-phenoxyphenyl)-1H-pyrazolo[4,3-d]pyrimidin-5-yl)methanol (50 mg, 0.15 mmol) and 4,4-difluoropiperidine-1-carbonitrile (87.9 mg, 0.6 mmol) in THF (2 mL) was added ZnCl₂ (82.0 mg, 0.6 mmol) at 25° C. under N₂ atmosphere. The reaction was stirred at 50° C. for 16 h. The reaction was added water (20 mL) and extracted with EtOAc (20 mL×2). The organic layers were washed with brine (20 mL), dried and concentrated. The crude was purified by TLC to give title compound (2.8 mg). ESI-MS (EI⁺, m/z): 479.21.

### Example 46. Preparation of Compound E1

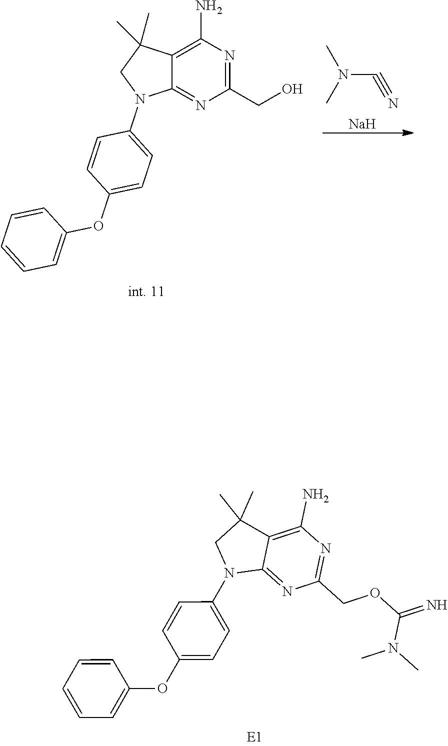

A mixture of (4-amino-5,5-dimethyl-7-(4-phenoxyphenyl)-6,7-dihydro-5H-pyrrolo[2,3-d]pyrimidin-2-yl)methanol (20 mg, 0.06 mmol) in DME (2 mL) was added NaH (2.8 mg, 0.07 mmol, 60%) and stirred for 0.5 h under N₂ atmosphere at room temperature. The mixture was concentrated. The residue was dissolved in N,N-dimethylcyanamide (0.5 mL) and stirred at room temperature for 0.5 h. The mixture was purified by Prep-HPLC to give title compound (4.7 mg). ESI-MS (EI⁺, m/z): 433.30. ¹H NMR (400 MHz, Chloroform-d) δ 8.74 (s, 1H), 7.64 (d, J=8.7 Hz, 2H), 7.32 (t, J=7.8 Hz, 2H), 7.10-6.95 (m, 5H), 5.63 (s, 2H), 5.16 (s, 2H), 3.74 (s, 2H), 3.11 (s, 6H), 1.43 (s, 6H).

### Example 47. Preparation of Compound E2 and Other Compounds

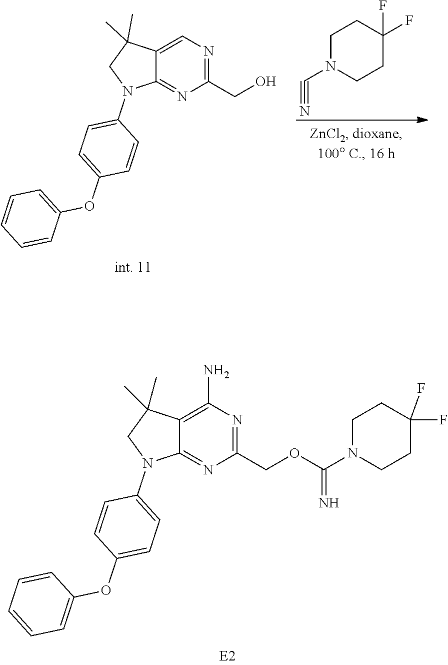

Synthesis of compound E2. To a solution of (4-amino-5,5-dimethyl-7-(4-phenoxyphenyl)-6,7-dihydro-5H-pyrrolo[2,3-d]pyrimidin-2-yl)methanol (600 mg, 1.26 mmol) and 4,4-difluoropiperidine-1-carbonitrile (737 mg, 5.04 mmol) in 1,4-dioxane (15 mL) was added ZnCl₂ (687 mg, 5.04 mmol) at 25° C. under N₂ atmosphere. The reaction mixture was stirred at 100° C. for 16 h. The reaction was concentrated. The residue was purified by Prep-HPLC to give title compound (200 mg). ESI-MS (EI⁺, m/z): 509.28. ¹H NMR (500 MHz, CDCl₃) δ 7.63-7.56 (m, 2H), 7.28-7.21 (m, 2H), 7.01 (tt, J=7.5, 1.1 Hz, 1H), 6.97-6.89 (m, 4H), 4.96 (s, 2H), 4.81 (s, 2H), 3.73 (s, 2H), 3.55 (t, J=6.0 Hz, 4H), 1.95-1.85 (m, 4H), 1.37 (s, 6H).

Additional compounds were synthesized as D2, for example:

| Compound | Structure | LCMS, NMR |
|---|---|---|
| E3 | 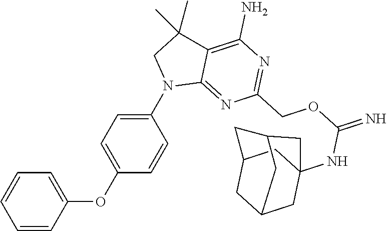 [SMILES: CC1(C)CN(c2ccc(Oc3ccccc3)cc2)c2nc(COC(=N)NC34CC5CC(CC(C5)C3)C4)nc(N)c21] | ESI-MS (EI+, m/z): 539.36. |
| E4 | 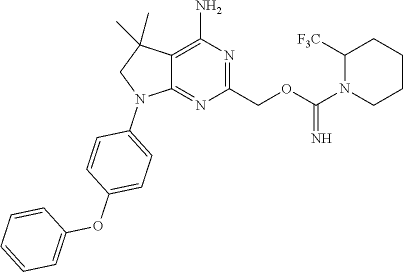 [SMILES: CC1(C)CN(c2ccc(Oc3ccccc3)cc2)c2nc(COC(=N)N3CCCCC3C(F)(F)F)nc(N)c21] | ESI-MS (EI+, m/z): 541.31. ¹H NMR (400 MHz, Chloroform-d) δ 7.83-7.46 (m, 2H), 7.29-7.20 (m, 2H), 7.04-6.89 (m, 5H), 4.97 (d, J = 13.8 Hz, 1H), 4.90-4.76 (m, 2H), 4.57 (s, 2H), 3.95 (d, J = 13.4 Hz, 1H), 3.67 (s, 2H), 2.98 (t, J = 13.0 Hz, 1H), 2.14 (d, J = 7.7 Hz, 1H), 1.96 (d, J = 15.1 Hz, 1H), 1.59 (q, J = 14.9, 13.6 Hz, 5H), 1.37 (s, 6H). |

### Example 48. Preparation of Compounds E5 and E6

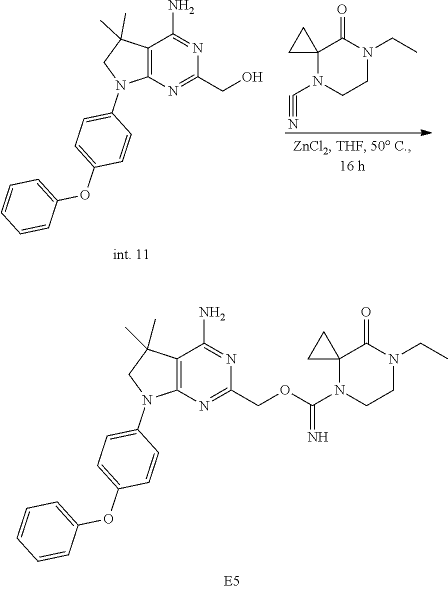 [SMILES: CC1(C)CN(c2ccc(Oc3ccccc3)cc2)c2nc(CO)nc(N)c21 ; CCN1CCN(C#N)C2(CC2)C1=O ; CCN1CCN(C(=N)OCc2nc(N)c3c(n2)N(c2ccc(Oc4ccccc4)cc2)CC3(C)C)C2(CC2)C1=O]

To a solution of (4-amino-5,5-dimethyl-7-(4-phenoxyphenyl)-6,7-dihydro-5H-pyrrolo[2,3-d]pyrimidin-2-yl)methanol (300 mg, 0.83 mmol) and 7-ethyl-8-oxo-4,7-diazaspiro[2.5]octane-4-carbonitrile (446 mg, 2.49 mmol) in THF (20 mL) was added ZnCl₂ (453 mg, 3.32 mmol) at 50° C. The reaction was stirred at 50° C. for 16 h. The reaction was concentrated. The residue was purified by Prep-HPLC to give title compound (100 mg). ESI-MS (EI⁺, m/z): 542.33. ¹H NMR (400 MHz, CDCl₃) δ 7.74-7.55 (m, 2H), 7.32-7.21 (m, 2H), 7.08-6.96 (m, 1H), 6.97-6.83 (m, 4H), 5.04 (s, 2H), 4.57 (s, 2H), 3.79 (t, J=5.6 Hz, 2H), 3.65 (s, 2H), 3.32 (t, J=5.6 Hz, 2H), 3.24 (q, J=7.2 Hz, 2H), 1.52 (q, J=4.4 Hz, 2H), 1.36 (s, 6H), 1.04-0.94 (m, 4H).

In some embodiments, compound E6 was prepared as E5.

| Compound | Structure | LCMS, NMR |
|---|---|---|
| E6 | 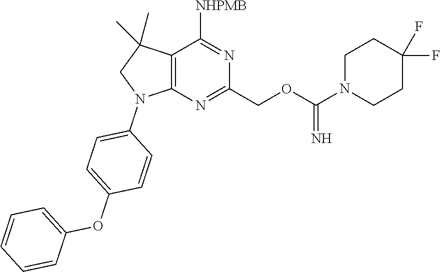 | ESI-MS (EI+, m/z) : 629.30. |

### Example 49. Preparation of Compound E7

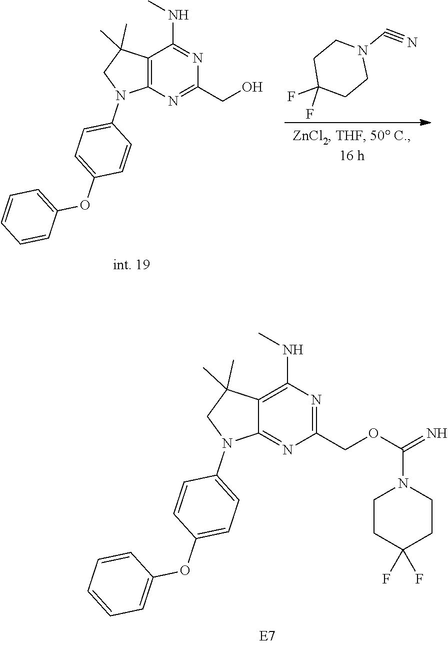 [SMILES: CNc1nc(CO)nc2c1C(C)(C)CN2c1ccc(Oc2ccccc2)cc1 ; N#CN1CCC(F)(F)CC1 ; CNc1nc(COC(=N)N2CCC(F)(F)CC2)nc2c1C(C)(C)CN2c1ccc(Oc2ccccc2)cc1]

To a solution of (5,5-dimethyl-4-(methylamino)-7-(4-phenoxyphenyl)-6,7-dihydro-5H-pyrrolo[2,3-d]pyrimidin-2-yl)methanol (50 mg, 0.13 mmol) and 4,4-difluoropiperidine-1-carbonitrile (78 mg, 0.53 mmol) in THF (1 mL) was added ZnCl₂ (72 mg, 0.53 mmol) under N₂ atmosphere. The reaction was stirred at 50° C. for 16 h. The reaction was concentrated. The residue was purified by Prep-HPLC to give title compound (20 mg). ESI-MS (EI⁺, m/z): 523.4 [M+H]⁺. ¹H NMR (500 MHz, CDCl₃) δ 10.50 (brs, 1H), 7.56-7.42 (m, 2H), 7.33-7.22 (m, 2H), 7.11-6.87 (m, 5H), 6.29 (brs, 1H), 5.21 (s, 2H), 3.92-3.55 (m, 6H), 2.95 (s, 3H), 2.25-1.75 (m, 4H), 1.37 (s, 6H).

### Example 50. Preparation of Compound E8

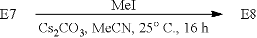

To a solution of (5,5-dimethyl-4-(methylamino)-7-(4-phenoxyphenyl)-6,7-dihydro-5H-pyrrolo[2,3-d]pyrimidin-2-yl)methyl 4,4-difluoropiperidine-1-carbimidate (80 mg, 0.15 mmol) and Cs₂CO₃ (150 mg, 0.46 mmol) in MeCN (3 mL) was added Mel (43 mg, 0.3 mmol) under N₂ atmosphere. The reaction was stirred at 25° C. for 16 h. The reaction was concentrated. The crude was purified by Prep-HPLC to give title product (6.6 mg). ESI-MS (EI+, m/z): 537.3. ¹H NMR (400 MHz, CDCl₃) δ 7.63-7.59 (m, 2H), 7.28-7.22 (m, 2H), 7.03-6.88 (m, 5H), 5.28 (s, 2H), 4.28 (s, 2H), 3.43-3.33 (m, 4H), 2.96 (d, J=4.8, 3H), 2.88 (s, 3H), 1.90-1.85 (m, 4H), 1.34 (s, 6H).

### Example 51. Preparation of Compound E9

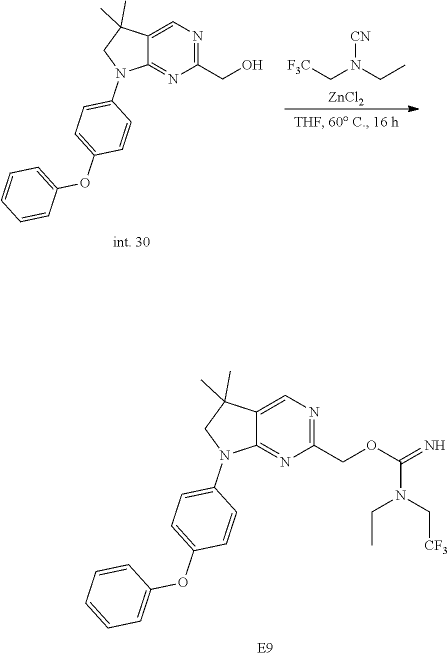

To a solution of [5,5-dimethyl-7-(4-phenoxyphenyl)-6H-pyrrolo[2,3-d]pyrimidin-2-yl]methanol (50 mg, 0.14 mmol) and ethyl(2,2,2-trifluoroethyl)cyanamide (66 mg, 0.43 mmol) in THF (10 mL) was added ZnCl₂ (78 mg, 0.58 mmol). The mixture was stirred at 60° C. for 16 h under N₂ atmosphere. The solvent was removed to yield a residue which was purified by Prep-HPLC to give title compound (49.8 mg). ESI-MS (EI⁺, m/z): 499.9 [M+H]⁺. ¹H NMR (400 MHz, CDCl₃) δ 8.05 (s, 1H), 7.81-7.71 (m, 2H), 7.37-7.29 (m, 2H), 7.13-6.96 (m, 5H), 5.18 (s, 2H), 3.97 (q, J=8.8 Hz, 2H), 3.81 (s, 2H), 3.45 (q, J=7.2 Hz, 2H), 1.43 (s, 6H), 1.18 (t, J=7.2 Hz, 3H).

### Example 52. Preparation of Compound E10

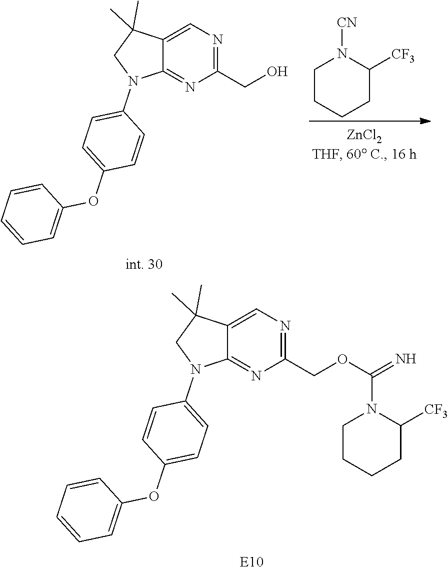

To a solution of [5,5-dimethyl-7-(4-phenoxyphenyl)-6H-pyrrolo[2,3-d]pyrimidin-2-yl]methanol (50 mg, 0.14 mmol) and 2-(trifluoromethyl)piperidine-1-carbonitrile (77 mg, 0.43 mmol) in THF (10 mL) was added ZnCl₂ (78 mg, 0.58 mmol). The mixture was stirred at 60° C. for 16 h under N₂ atmosphere. The solvent was removed to yield a residue which was purified by Prep-HPLC to give title compound (46.8 mg). ESI-MS (EI⁺, m/z): 526.2 [M+H]⁺. ¹H NMR (400 MHz, CDCl₃) δ 8.06 (s, 1H), 7.84-7.72 (m, 2H), 7.37-7.29 (m, 2H), 7.14-6.95 (m, 5H), 5.24-5.05 (m, 2H), 4.94-4.83 (m, 1H), 3.99 (d, J=13.2 Hz, 1H), 3.81 (s, 2H), 3.05 (t, J=12.8 Hz, 1H), 2.02-1.90 (m, 1H), 1.77-1.59 (m, 4H), 1.52-1.38 (m, 7H).

### Example 53. Preparation of Compound E11

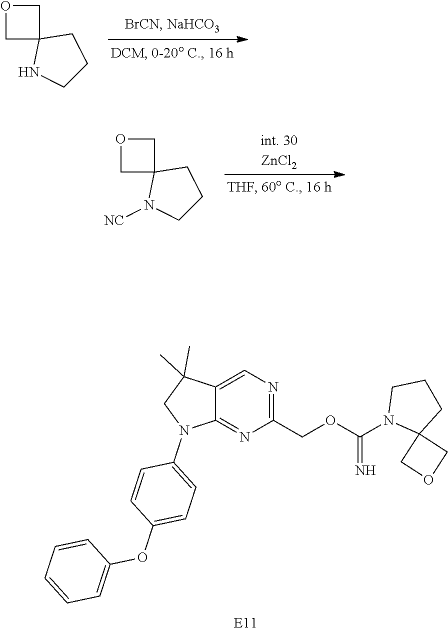 [SMILES: C1CNC2(C1)COC2 ; N#CN1CCCC12COC2 ; CC1(C)CN(c2ccc(Oc3ccccc3)cc2)c2nc(COC(=N)N3CCCC34COC4)ncc21]

1. Synthesis of compound 2-oxa-5-azaspiro[3.4]octane-5-carbonitrile. To a mixture of 2-oxa-5-azaspiro[3.4]octane; oxalic acid (30 mg, 0.15 mmol) and saturated aqueous NaHCO₃ (0.5 mL) in DCM (0.5 mL) was added the mixture BrCN (20 mg, 0.19 mmol) in DCM (0.5 mL) at 0° C. under N₂ atmosphere. Then the mixture was stirred at 20° C. for 16 h. The reaction mixture was diluted with H₂O (10 mL) and extracted with DCM (10 mL×3). The combined organic phases were washed with brine (10 mL), dried over anhydrous Na₂SO₄, filtered and concentrated under vacuum to give crude 2-oxa-5-azaspiro[3.4]octane-5-carbonitrile (20 mg). ESI-MS (EI⁺, m/z): 139.1 [M+H]⁺.

2. Synthesis of compound E11. To a solution of (5,5-dimethyl-7-(4-phenoxyphenyl)-6,7-dihydro-5H-pyrrolo[2,3-d]pyrimidin-2-yl)methanol (40 mg, 0.12 mmol) and 2-oxa-5-azaspiro[3.4]octane-5-carbonitrile (32 mg, 0.23 mmol) in THF (2 mL) was added ZnCl₂ (63 mg, 0.46 mmol). The mixture was stirred at 60° C. for 16 h. The reaction mixture was filtered and concentrated. The crude was purified by Prep-HPLC to give title compound (13.5 mg). ESI-MS (EI⁺, m/z): 486.2 [M+H]⁺. ¹H NMR (400 MHz, CDCl₃) δ 8.06 (s, 1H), 7.80-7.73 (m, 2H), 7.33 (m, J=8.8, 7.2 Hz, 2H), 7.12-7.07 (m, 1H), 7.02-6.97 (m, 4H), 5.44 (d, J=6.0 Hz, 2H), 5.37 (s, 2H), 4.40 (d, J=6.0 Hz, 2H), 3.80 (s, 2H), 3.45 (t, J=6.4 Hz, 2H), 2.31 (t, J=6.8 Hz, 2H), 1.79-1.72 (m, 2H), 1.43 (s, 6H).

### Example 54. Preparation of Compound E12

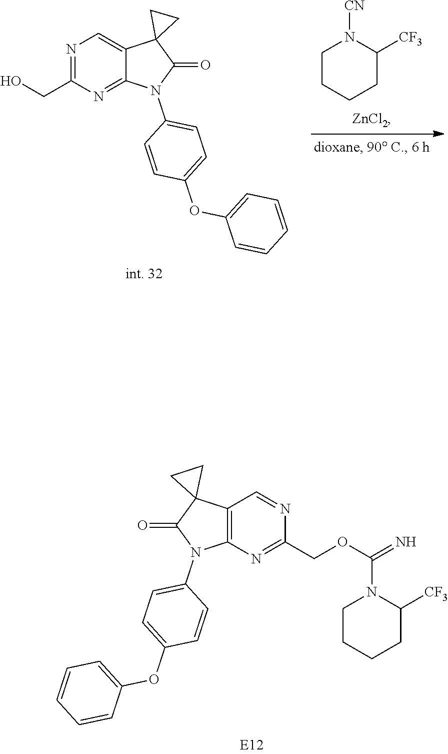 [SMILES: O=C1N(c2ccc(Oc3ccccc3)cc2)c2nc(CO)ncc2C12CC2 ; N#CN1CCCCC1C(F)(F)F ; N=C(OCc1ncc2c(n1)N(c1ccc(Oc3ccccc3)cc1)C(=O)C21CC1)N1CCCCC1C(F)(F)F]

To a solution of 2′-(hydroxymethyl)-7′-(4-phenoxyphenyl)spiro[cyclopropane-1,5′-pyrrolo[2,3-d]pyrimidin]-6′(7′H)-one (30 mg, 0.083 mmol) and 2-(trifluoromethyl)piperidine-1-carbonitrile (30 mg, 0.17 mmol) in anhydrous 1,4-dioxane (2 mL) was added ZnCl₂ (34 mg, 0.25 mmol) under N₂ atmosphere. The mixture was stirred at 90° C. for 6 h. The reaction mixture was concentrated under reduced pressure to give a residue. The residue was purified by Prep-HPLC to give title compound (11 mg). ESI-MS (EI⁺, m/z): 538.2 [M+H]⁺. ¹H NMR (400 MHz, CDCl₃) δ 8.11 (brs, 1H), 7.49 (d, J=8.8 Hz, 2H), 7.44-7.35 (m, 2H), 7.21-7.14 (m, 1H), 7.13-7.07 (m, 4H), 5.39-5.13 (m, 2H), 4.84-4.72 (m, 1H), 3.91-3.78 (m, 1H), 3.11-2.98 (m, 1H), 2.05-1.94 (m, 3H), 1.88-1.75 (m, 2H), 1.71-1.64 (m, 4H), 0.91-0.84 (m, 2H).

### Example 55. Preparation of Compound E13

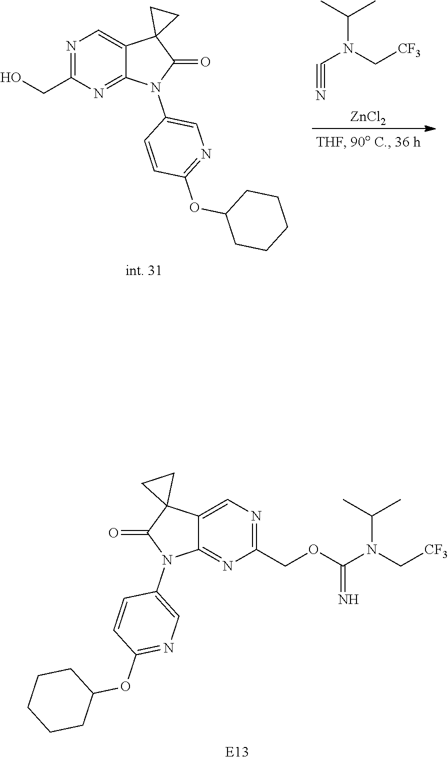

To a solution of 7′-(6-(cyclohexyloxy)pyridin-3-yl)-2′-(hydroxymethyl)spiro[cyclopropane-1,5′-pyrrolo[2,3-d]pyrimidin]-6′(7′H)-one (35 mg, 0.096 mmol) and isopropyl(2,2,2-trifluoroethyl)cyanamide (63 mg, 0.38 mmol) in dioxane (3 mL) was added ZnCl₂ (52 mg, 0.38 mmol). The mixture was stirred at 90° C. for 36 h under N₂ atmosphere. The mixture was concentrated. The residue was purified by Prep-HPLC to give title compound (20 mg). ESI-MS (EI⁺, m/z): 533.0 [M+H]⁺. ¹H NMR (400 MHz, CDCl₃) δ 8.36-8.25 (m, 1H), 8.12-8.00 (m, 1H), 7.78-7.62 (m, 1H), 6.88-6.75 (m, 1H), 5.38-5.28 (m, 2H), 5.12-5.02 (m, 1H), 4.21-4.10 (m, 1H), 3.91-3.82 (m, 2H), 2.04 (dd, J=3.2, 12.0 Hz, 2H), 2.00-1.97 (m, 2H), 1.86-1.80 (m, 2H), 1.80-1.77 (m, 2H), 1.65-1.56 (m, 2H), 1.54-1.46 (m, 2H), 1.43-1.29 (m, 2H), 1.17 (d, J=6.8 Hz, 6H).

### Example 56. Preparation of Compound E14

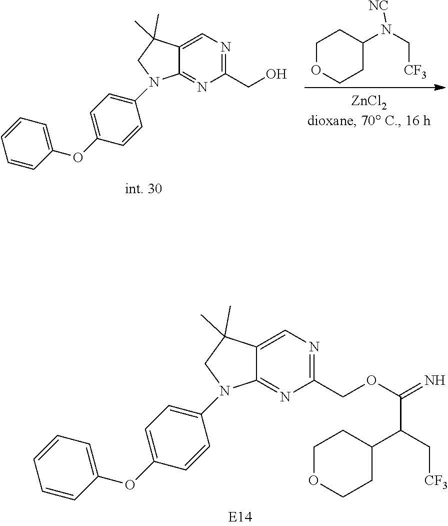 [SMILES: CC1(C)CN(c2ccc(Oc3ccccc3)cc2)c2nc(CO)ncc21 ; N#CN(CC(F)(F)F)C1CCOCC1 ; CC1(C)CN(c2ccc(Oc3ccccc3)cc2)c2nc(COC(=N)C(CC(F)(F)F)C3CCOCC3)ncc21]

To a solution of [5,5-dimethyl-7-(4-phenoxyphenyl)-6H-pyrrolo[2,3-d]pyrimidin-2-yl]methanol (50 mg, 0.14 mmol) and tetrahydropyran-4-yl(2,2,2-trifluoroethyl)cyanamide (120 mg, 0.58 mmol) in anhydrous 1,4-dioxane (4 mL) was added ZnCl₂ (98 mg, 0.72 mmol) at 25° C. under N₂ atmosphere. The mixture was stirred at 70° C. for 12 h. The reaction mixture was concentrated. The residue was purified by Prep-HPLC to give title compound (6.2 mg). ESI-MS (EI⁺, m/z): 556.4 [M+H]⁺. ¹H NMR (400 MHz, DMSO-d₆) δ 8.21 (s, 1H), 7.88 (d, J=9.2 Hz, 2H), 7.38 (m, 2H), 7.12 (m, 1H), 7.07 (d, J=9.2 Hz, 2H), 6.98 (d, J=7.6 Hz, 2H), 6.19 (s, 1H), 5.10 (s, 2H), 4.13 (q, J=9.6 Hz, 2H), 3.99 (s, 1H), 3.87 (s, 2H), 3.82 (dd, J=11.6, 4.4 Hz, 2H), 3.26 (d, J=11.6 Hz, 2H), 1.72 (d, J=12.8 Hz, 2H), 1.57 (d, J=12.0 Hz, 2H), 1.39 (s, 6H).

### Example 57. Preparation of Compound F1

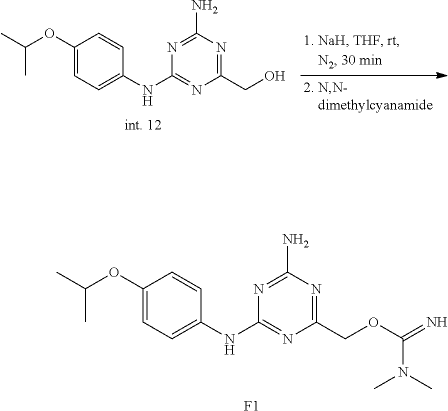 [SMILES: CC(C)Oc1ccc(Nc2nc(N)nc(CO)n2)cc1 ; CC(C)Oc1ccc(Nc2nc(N)nc(COC(=N)N(C)C)n2)cc1 ; "F1"]

A mixture of (4-amino-6-((4-isopropoxyphenyl)amino)-1,3,5-triazin-2-yl)methanol (50 mg, 0.18 mmol) and NaH (8.72 mg, 0.22 mmol, 60%) in THF (1 mL) was stirred at room temperature for 30 min under N₂ atmosphere, then N,N-dimethylcyanamide (127 mg, 1.8 mmol) was added. The reaction was stirred for 30 min. The reaction was filtered and the residue was purified by Prep-HPLC to afford title compound (3.4 mg). ESI-MS (EI⁺, m/z): 346.10.

### Example 58. Preparation of Compound F2

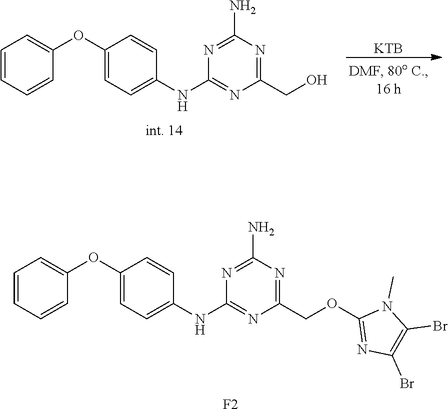 [SMILES: Nc1nc(CO)nc(Nc2ccc(Oc3ccccc3)cc2)n1 ; "KTB" ; Cn1c(OCc2nc(N)nc(Nc3ccc(Oc4ccccc4)cc3)n2)nc(Br)c1Br ; "F2"]

To a solution of (4-amino-6-[(4-phenoxy-phenyl)-amino]-1,3,5-triazin-2-yl)-methanol (200 mg, 0.65 mmol) and 2, 4, 5-tribromo-1-methyl-1H-imidazole (210 mg, 0.65 mmol) in DMF (2 mL) was added potassium tert-butoxide (95 mg, 0.85 mmol) at 0° C. The mixture was allowed to warm to room temperature, then stirred at 80° C. for 16 h. The reaction mixture was quenched with water (50 mL), extracted with ethyl acetate (3×15 mL). The organic portion was washed with brine (3×15 mL), dried and concentrated. The residue was purified by TLC to give title compound (10.0 mg). ESI-MS (EI⁺, m/z) 546.07. ¹H NMR (400 MHz, DMSO-d6) δ 9.54 (s, 1H), 7.67 (s, 2H), 7.41-7.30 (m, 3H), 7.18-7.06 (m, 3H), 6.98-6.88 (m, 5H), 5.08 (s, 2H).

### Example 59. Preparation of Compound F3

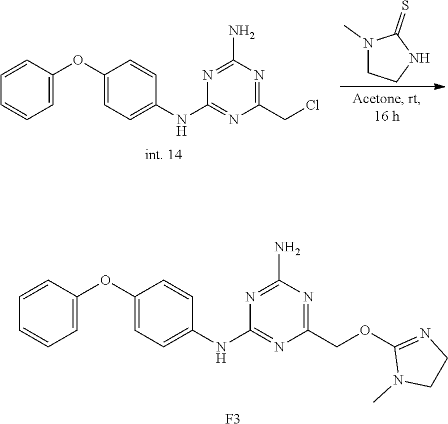

To a solution of 6-(chloromethyl)-N2-(4-phenoxyphenyl)-1,3,5-triazine-2,4-diamine (50 mg, 0.15 mmol) and 1-methylimidazolidine-2-thione (8.53 mg, 0.3 mmol) in 1 mL of acetone was stirred at room temperature for 16 h. The mixture was purified by Prep-HPLC to give title compound (20.4 mg). ¹H NMR (400 MHz, DMSO-d₆) δ 9.60 (s, 1H), 7.77 (d, J=8.8 Hz, 2H), 7.36 (t, J=8.0 Hz, 2H), 7.26-7.06 (m, 3H), 7.04-N6.90 (i, 4H), 4.15 (s, 2H), 3.62 (t, J=9.2 Hz, 2H), 3.40 (s, 2H), 2.78 (s, 3H).

### Example 60. Preparation of Compound F4 and Other Compounds

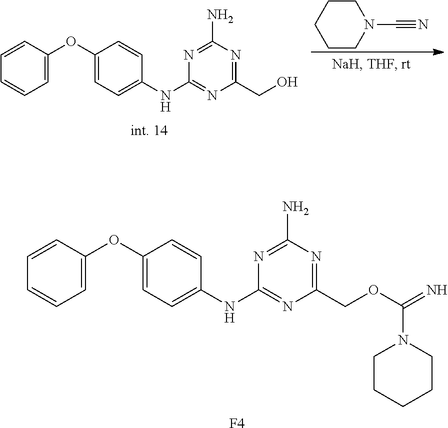 [SMILES: Nc1nc(CO)nc(Nc2ccc(Oc3ccccc3)cc2)n1 ; N#CN1CCCCC1 ; N=C(OCc1nc(N)nc(Nc2ccc(Oc3ccccc3)cc2)n1)N1CCCCC1 ; "F4"]

To a solution of (4-amino-6-((4-phenoxyphenyl)amino)-1,3,5-triazin-2-yl)methanol (100 mg, 0.32 mmol) and NaH (8.53 mg, 0.35 mmol) in 2 mL of THF was stirred at room temperature for 2 h in an inert atmosphere. The reaction mixture was concentrated, the residue was added into piperidine-1-carbonitrile (0.5 mL) and stirred at room temperature for another 1 h. The mixture was purified by Prep-HPLC to give title compound (4.4 mg). ¹H NMR (400 MHz, DMSO-d₆) δ 9.48 (s, 1H), 7.77 (d, J=9.0 Hz, 2H), 7.40 7.31 (m, 2H), 7.12-6.88 (m, 7H), 5.72 (s, 8H), 4.80 (s, 2H), 3.26 (d, J=5.4 Hz, 4H), 1.44 (s, 6H).

Additional compounds were synthesized as compound F4, for example:

| Compound | Structure | LCMS, NMR |
|---|---|---|
| F5 | 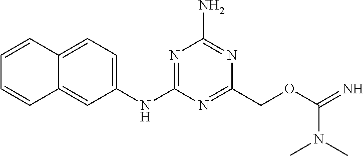 [SMILES: CN(C)C(=N)OCc1nc(N)nc(Nc2ccc3ccccc3c2)n1] | ESI-MS (EI+, m/z): 338.25. ¹H NMR (400 MHz, DMSO-d₆) δ 9.73 (s, 1H), 8.44 (s, 1H), 8.28 (s, 1H), 7.79 (q, J = 8.6 Hz, 4H), 7.46 (ddd, J = 8.1, 6.7, 1.3 Hz, 1H), 7.36 (ddd, J = 8.1, 6.8, 1.2 Hz, 1H), 7.21 (s, 2H), 4.96 (s, 2H), 2.91 (s, 6H). |
| F6 | 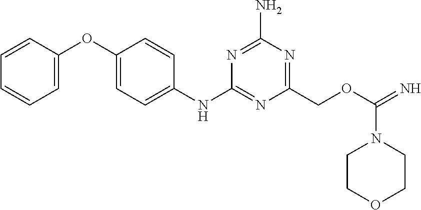 [SMILES: N=C(OCc1nc(N)nc(Nc2ccc(Oc3ccccc3)cc2)n1)N1CCOCC1] | ESI-MS (EI+, m/z): 422.30. ¹H NMR (400 MHz, DMSO-d₆) δ 9.47 (s, 1H), 7.76 (d, J = 8.7 Hz, 2H), 7.38-7.34 (m, 2H), 7.08 (t, J = 7.4 Hz, 2H), 6.95 (dd, J = 8.5, 4.3 Hz, 5H), 5.85 (s, 1H), 4.84 (s, 2H), 3.56 (t, J = 4.9 Hz, 4H), 3.22 (t, J = 4.8 Hz, 4H). |
| F7 | 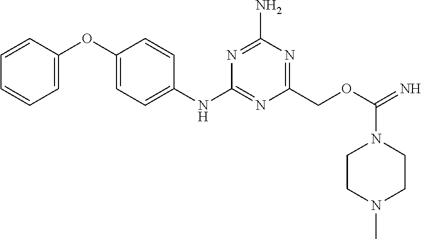 [SMILES: CN1CCN(C(=N)OCc2nc(N)nc(Nc3ccc(Oc4ccccc4)cc3)n2)CC1] | ESI-MS (EI+, m/z): 435.35. ¹H NMR (400 MHz, DMSO-d₆) δ 9.59 (s, 1H), 9.41 (s, 2H), 7.70 (s, 2H), 7.44-7.33 (m, 2H), 7.23 (d, J = 24.8 Hz, 1H), 7.10 (td, J = 7.3, 1.2 Hz, 1H), 6.97 (td, J = 8.8, 1.6 Hz, 4H), 5.26 (s, 2H), 3.38 (s, 8H), 2.84 (s, 3H). |
| F9 | 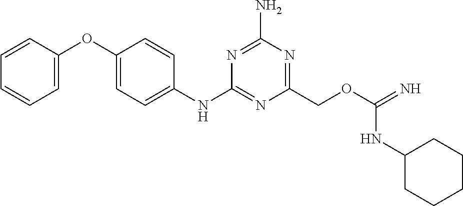 [SMILES: N=C(NC1CCCCC1)OCc1nc(N)nc(Nc2ccc(Oc3ccccc3)cc2)n1] | ¹H NMR (400 MHz, DMSO-d₆) δ 9.54 (s, 1H), 8.31 (s, 1H), 7.75 (d, J = 8.6 Hz, 2H), 7.38-7.33 (m, 2H), 7.19- 6.87 (m, 8H), 4.94 (s, 2H), 1.73 (d, J = 10.5 Hz, 2H), 1.62 (s, 3H), 1.22 (d, J = 11.2 Hz, 6H). |

### Example 61. Preparation of Compound F8

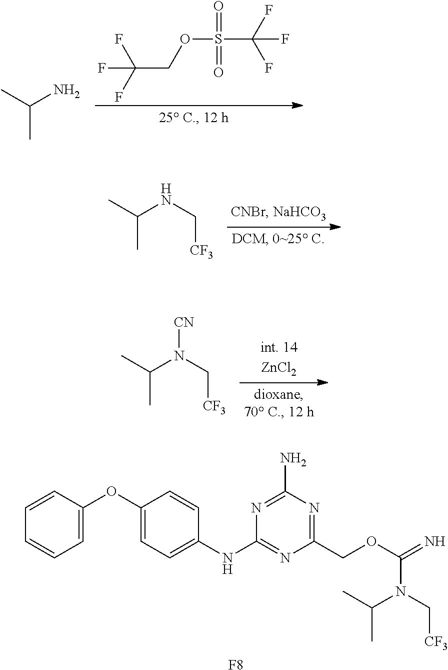

1. Synthesis of N-(2,2,2-trifluoroethyl)propan-2-amine. A solution of 2,2,2-trifluoroethyl trifluoromethanesulfonate (5.0 g, 21.5 mmol) in propan-2-amine (5.52 g, 93.4 mmol) was stirred at 25° C. for 12 h. The reaction mixture was distilled at 80° C. (oil bath) to give title compound (0.5 g). ¹H NMR (400 MHz, CDCl₃) δ 3.20 (q, J=9.6 Hz, 2H), 2.96 (p, J=6.4 Hz, 1H), 1.30 (d, J=6.4 Hz, 1H), 1.10 (d, J=6.4 Hz, 6H).

2. Synthesis of N-isopropyl-N-(2,2,2-trifluoroethyl)cyanamide. To a solution of N-(2,2,2-trifluoroethyl)propan-2-amine (50 mg, 0.35 mmol) in anhydrous DCM (1 mL) and saturated aq. NaHCO₃ (1 mL) was added dropwise a solution of BrCN (75 mg, 0.71 mmol) in DCM (1 mL) at 0° C. The mixture was stirred at 25° C. for 12 h. The reaction mixture was diluted with saturated NaHCO₃ (10 mL) and extracted with DCM (10 mL×3). The combined organic phase was washed with brine (10 mL), dried over anhydrous Na₂SO₄, filtered and concentrated in vacuum at 20° C. to give title compound (40 mg crude).

3. Synthesis of compound F8. To a solution of [4-amino-6-(4-phenoxyanilino)-1,3,5-triazin-2-yl]methanol (10 mg, 0.032 mmol) and isopropyl(2,2,2-trifluoroethyl)cyanamide (16 mg, 0.097 mmol) in anhydrous 1,4-dioxane (1 mL) was added ZnCl₂ (18 mg, 0.13 mmol) under N₂ atmosphere. The mixture was stirred at 70° C. for 12 h. The reaction mixture was concentrated. The residue was purified by Prep-HPLC to give title compound (4.5 mg). ESI-MS (EI⁺, m/z): 476.1 [M+H]⁺.

### Example 62. Preparation of Compound F10 and Other Compounds

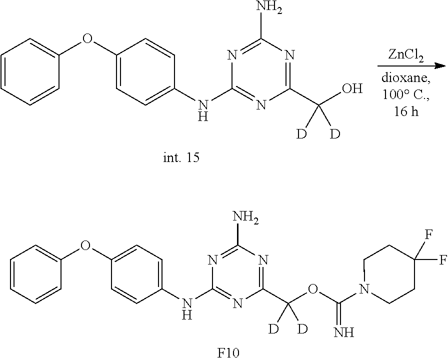 [SMILES: [2H]C([2H])(O)c1nc(N)nc(Nc2ccc(Oc3ccccc3)cc2)n1 ; "ZnCl2" ; [2H]C([2H])(OC(=N)N1CCC(F)(F)CC1)c1nc(N)nc(Nc2ccc(Oc3ccccc3)cc2)n1 ; "F10"]

To a solution of (4-amino-6-((4-phenoxy-phenyl)-amino)-1, 3, 5-triazin-2-yl)-methan-d₂-ol (200 mg, 0.64 mmol) and 4,4-difluoropiperidine-1-carbonitrile (376 mg, 2.57 mmol) in Dioxane (4 mL) was added zinc dichloride (350 mg, 2.57 mmol). The resulting mixture was heated up to 100° C. and stirred for 16 h, poured into water (30 mL), extracted with ethyl acetate (3×20 mL). The combined organic layers were washed with brine (3×30 mL), dried and concentrated. The residue was purified by flash column chromatography to give title compound (13.0 mg). ESI-MS (EI⁺, m/z): 458.19.

Additional compounds were synthesized as F10, for example:

| Compound | Structure | LCMS, NMR |
|---|---|---|
| F11 | 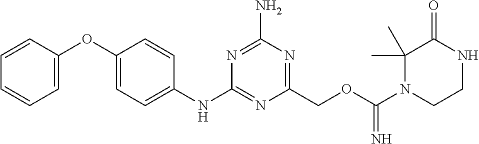 [SMILES: CC1(C)C(=O)NCCN1C(=N)OCc1nc(N)nc(Nc2ccc(Oc3ccccc3)cc2)n1] | ESI-MS (EI+, m/z): 463.21. |
| F12 | 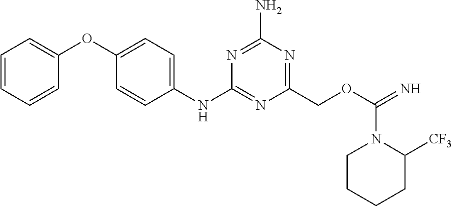 [SMILES: N=C(OCc1nc(N)nc(Nc2ccc(Oc3ccccc3)cc2)n1)N1CCCCC1C(F)(F)F] | ESI-MS (EI+, m/z): 488.19. ¹H NMR (400 MHz, Chloroform-d) δ 7.53-7.33 (m, 2H), 7.30-7.22 (m, 2H), 7.19 (s, 1H), 7.02 (tt, J = 7.3, 1.1 Hz, 1H), 6.96-6.86 (m, 4H), 5.28 (d, J = 37.8 Hz, 2H), 4.95 (d, J = 14.9 Hz, 1H), 4.85-4.67 (m, 2H), 3.76 (d, J = 13.0 Hz, 1H), 2.99 (t, J = 12.8 Hz, 1H), 1.93 (d, J = 13.0 Hz, 1H), 1.73-1.55 (m, 5H), 1.46 (d, J = 16.9 Hz, 1H). |

### Example 63. Preparation of Compound F13

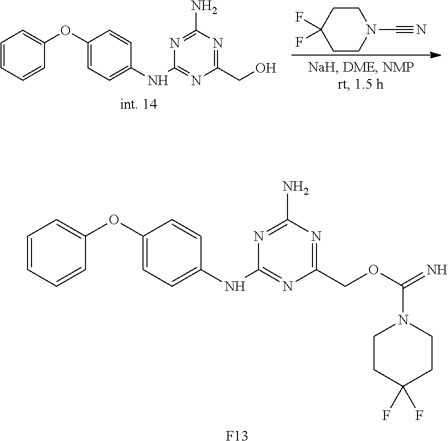

To a solution of (4-amino-6-((4-phenoxyphenyl)amino)-1,3,5-triazin-2-yl)methanol (200 mg, 0.65 mmol) in NMP (0.6 mL) and DME (0.9 mL) was added NaH (60%, 34 mg, 0.84 mmol). The mixture was stirred at room temperature under N₂ for 0.5 h, then 4,4-difluoropiperidine-1-carbonitrile (944 mg, 6.47 mmol) was added and stirred for 1 h. The reaction was purified by Prep-HPLC to afford title compound (20 mg). ESI-MS (EI⁺, m/z): 456.30. ¹H NMR (400 MHz, DMSO-d₆) δ 9.51 (s, 1H), 8.23 (s, 1H), 7.74 (d, J=8.4 Hz, 2H), 7.36 (t, J=7.9 Hz, 2H), 7.09 (t, J=7.4 Hz, 2H), 6.95 (d, J=8.4 Hz, 4H), 4.90 (s, 2H), 3.50 (t, J=5.7 Hz, 4H), 1.97 (td, J=13.9, 6.5 Hz, 4H).

### Example 64. Preparation of Compound F14

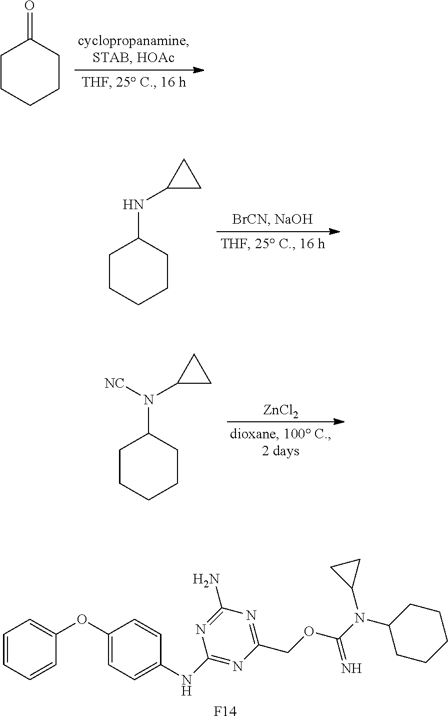

1. Synthesis of N-cyclopropylcyclohexanamine. To a solution of cyclohexanone (5.00 g, 50.9 mmol) in THF (50 mL) was added cyclopropylamine (3.20 g, 56.0 mmol), HOAc (3.06 g, 50.9 mmol) and STAB (16.2 g, 76.4 mmol). The reaction was stirred at room temperature for 16 h. The reaction mixture was poured into water (60 mL) and acidified to Ph=2 with 5 N HCl followed by removal of the organic portion. The aqueous portion was brought to pH>12 with 5 N NaOH and extracted with DCM (3×30 mL). The collected organic extracts were dried with sodium sulfate, filtered and the volatile organics were removed in vacuo to afford title compound (4.70 g). ¹H NMR (400 MHz, Chloroform-d) δ 2.51 (m, J=10.5, 3.8 Hz, 1H), 2.12-2.03 (m, 1H), 1.95-1.85 (m, 2H), 1.67 (dd, J=12.8, 5.8, 2.5 Hz, 3H), 1.56 (dd, J=12.3, 5.1, 3.2, 1.4 Hz, 1H), 1.22 (qt, J=12.5, 3.2 Hz, 2H), 1.13 (dt, J=12.3, 3.2 Hz, 1H), 1.02 (dd, J=23.2, 12.8, 10.0, 3.4 Hz, 2H), 0.41-0.35 (m, 2H), 0.30-0.25 (m, 2H).

2. Synthesis of N-cyclohexyl-N-cyclopropylcyanamide. To a solution of N-cyclopropyl-cyclohexanamine (1.00 g, 7.18 mmol) in THF (10 mL) were added NaOH (0.29 g, 7.18 mmol) and cyanogen bromide (0.76 g, 7.18 mmol). The reaction mixture was quenched with water. The resulting solution was extracted with ethyl acetate (3×30 mL). The organic portion was washed with brine (3×30 mL), dried over anhydrous sodium sulfate, filtered and concentrated in vacuo o afford title compound (0.50 g). ¹HNMR (400 MHz, Chloroform-d) δ 2.81 (m, J=11.4, 3.9 Hz, 1H), 2.66-2.59 (m, 1H), 1.98-1.90 (m, 2H), 1.84-1.74 (m, 2H), 1.60 (dt, J=12.7, 4.9, 3.1, 1.4 Hz, 1H), 1.46-1.32 (m, 2H), 1.24 (dd, J=19.0, 9.5, 7.6, 3.1 Hz, 2H), 1.17-1.06 (m, 1H), 0.71 (d, J=5.0 Hz, 4H).

Compound F14 was prepared as compound F10. LCMS (ESI): m/z, 474.25.

### Example 65. Preparation of Compound F15

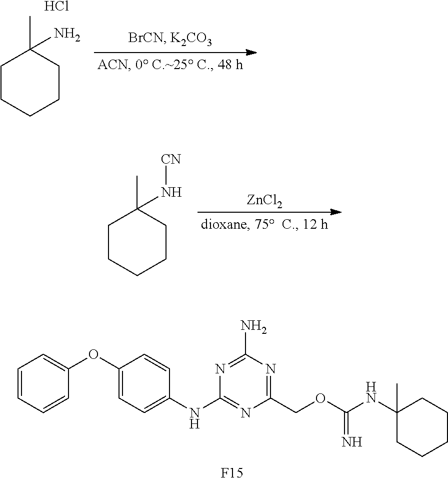

Synthesis of N-(1-methylcyclohexyl)cyanamide. To a solution of 1-methylcyclohexan-1-amine hydrochloride (400 mg, 2.67 mmol) in ACN (5 mL) were added K₂CO₃ (740 mg, 5.34 mmol) at 0° C., cyanogen bromide (310 mg, 2.94 mmol) was followed. The mixture was allowed to warm to 25° C. and stirred for 48 h, then quenched with water. The resulting solution was extracted with ethyl acetate (3×30 mL). The organic portion was washed with brine (3×30 mL), dried, filtered and concentrated in vacuo to afford title compound (350 mg). ESI-MS (EI⁺, m/z): 318.30 [2M+ACN+H]⁺.

Compound F15 was synthesized as compound F10. ESI-MS (EI⁺, m/z): 448.29.

### Example 66. Preparation of Compound F16

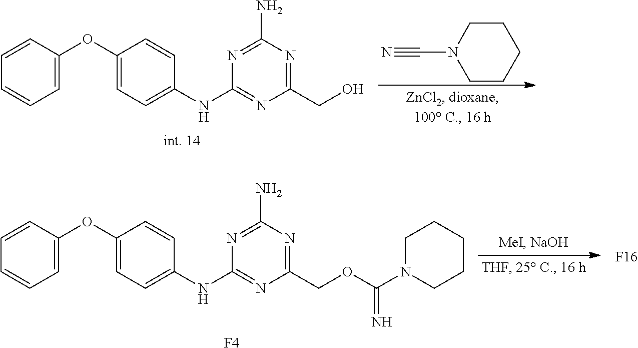 [SMILES: Nc1nc(CO)nc(Nc2ccc(Oc3ccccc3)cc2)n1 ; N#CN1CCCCC1 ; N=C(OCc1nc(N)nc(Nc2ccc(Oc3ccccc3)cc2)n1)N1CCCCC1 ; "F16" ; "F4"]

Compound F4 was synthesized as compound F10. ESI-MS (EI⁺, m/z): 420.21. To a solution of (4-amino-6-((4-phenoxyphenyl)amino)-1,3,5-triazin-2-yl)methyl piperidine-1-carbimidate (40 mg, 0.095 mmol) and NaOH (11.4 mg, 0.29 mmol) in THF (3 mL) and Water (1 mL) was added Mel (13.5 mg, 0.095 mmol) at 25° C. under N₂ atmosphere. The reaction was stirred at 25° C. for 16 h. The reaction was added water (20 mL) and extracted with EtOAc (15 mL×2). The organic layers were concentrated. The residue was purified by TLC to give title compound (3.5 mg). ESI-MS (EI+, m/z): 434.25.

### Example 67. Preparation of Compound F17

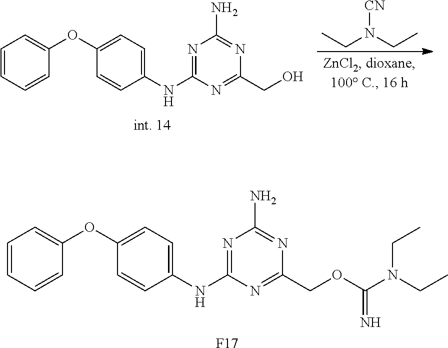

Compound F17 was synthesized as compound F10. ESI-MS (EI+, m/z): 408.3. ¹H NMR (400 MHz, DMSO-d₆) δ 9.51 (s, 1H), 7.72 (d, J=8.4 Hz, 2H), 7.38-7.33 (m, 2H), 7.16-7.00 (m, 2H), 6.96-6.92 (m, 4H), 4.98 (s, 2H), 3.32-3.30 (m, 4H), 1.06 (t, J=7.0 Hz, 6H).

### Example 68. Preparation of Compound F18

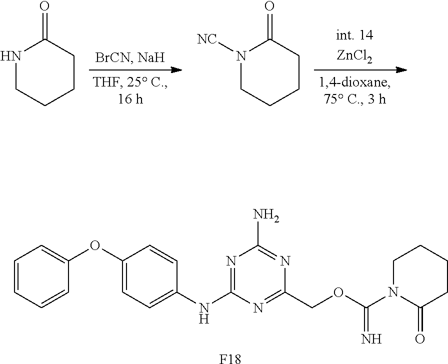 [SMILES: O=C1CCCCN1 ; N#CN1CCCCC1=O ; N=C(OCc1nc(N)nc(Nc2ccc(Oc3ccccc3)cc2)n1)N1CCCCC1=O ; "F18"]

1. Synthesis of 2-oxopiperidine-1-carbonitrile. To a solution of piperidin-2-one (1.2 g, 12.11 mmol) in THF (30 mL) was added sodium hydride (0.97 g, 24.22 mmol, 60%) at 0° C. and stirred for 30 min. A solution of cyanogen bromide (1.67 g, 15.77 mmol) in THF (5 mL) was added dropwise at 0° C. and the mixture was stirred at 25° C. for 16 h. The mixture was purified by Prep-TLC to yield title compound (50 mg). ¹H NMR (400 MHz, CDCl₃) b 3.96-3.76 (m, 2H), 2.55 (t, J=6.0 Hz, 2H), 1.97-1.84 (m, 4H).

2. Synthesis of compound F18. To a solution of (4-amino-6-[(4-phenoxyphenyl)amino]-1,3,5-triazin-2-yl)methanol (100 mg, 0.32 mmol) and 2-oxopiperidine-1-carbonitrile (52 mg, 0.42 mmol) in 1,4-dioxane (5 mL) was added zinc dichloride (170 mg, 1.28 mmol) under N₂ atmosphere. The mixture was stirred at 75° C. for 3 h. The solvent was removed and the residue was purified by Prep-TLC to yield title compound (9.8 mg). ESI-MS (EI⁺, m/z): 456.1 [M+Na]⁺. ¹H NMR (400 MHz, CDCl₃) δ 8.96 (s, 1H), 7.55-7.45 (m, 2H), 7.35-7.30 (m, 2H), 7.13-7.07 (m, 1H), 7.03-6.91 (m, 4H), 5.23 (s, 2H), 5.05 (s, 2H), 3.84-3.80 (m, 2H), 2.55-2.53 (m, 2H), 1.84-1.80 (m, 4H).

### Example 69. Preparation of Compound F19 and other compounds

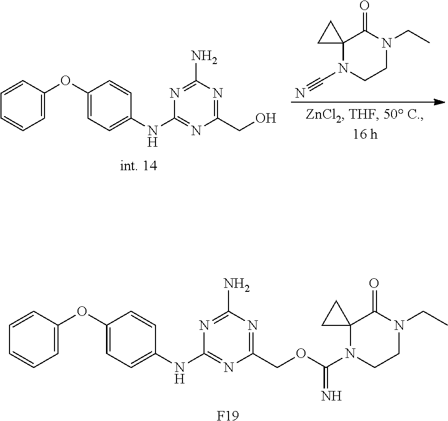 [SMILES: Nc1nc(CO)nc(Nc2ccc(Oc3ccccc3)cc2)n1 ; CCN1CCN(C#N)C2(CC2)C1=O ; CCN1CCN(C(=N)OCc2nc(N)nc(Nc3ccc(Oc4ccccc4)cc3)n2)C2(CC2)C1=O ; "F19"]

To a solution of (4-amino-6-((4-phenoxyphenyl)amino)-1,3,5-triazin-2-yl)methanol (50 mg, 0.16 mmol) and 7-ethyl-8-oxo-4,7-diazaspiro[2.5]octane-4-carbonitrile (114.7 mg, 0.64 mmol) in THF (1 mL) was added ZnCl₂ (87.2 mg, 0.64 mmol) at 25° C. under N₂ atmosphere. The reaction was stirred at 50° C. for 16 h. The reaction was added water (20 mL) and extracted with EtOAc (20 mL×2), dried and concentrated. The residue was purified by TLC to give title compound (27 mg).

Additional compounds were synthesized as F19, for example:

| Compound | Structure | LCMS, NMR |
|---|---|---|
| F20 | 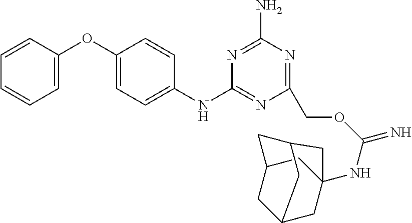 [SMILES: N=C(NC12CC3CC(CC(C3)C1)C2)OCc1nc(N)nc(Nc2ccc(Oc3ccccc3)cc2)n1] | ESI-MS (EI+, m/z): 486.28. |
| F21 | 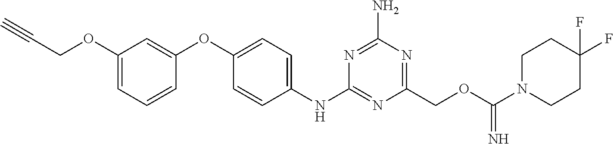 [SMILES: C#CCOc1cccc(Oc2ccc(Nc3nc(N)nc(COC(=N)N4CCC(F)(F)CC4)n3)cc2)c1] | ESI-MS (EI+, m/z): 510.21. |
| F22 | 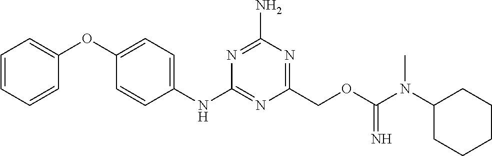 [SMILES: CN(C(=N)OCc1nc(N)nc(Nc2ccc(Oc3ccccc3)cc2)n1)C1CCCCC1] | ESI-MS (EI+, m/z): 448.24. |

### Example 70. Preparation of Compound F23

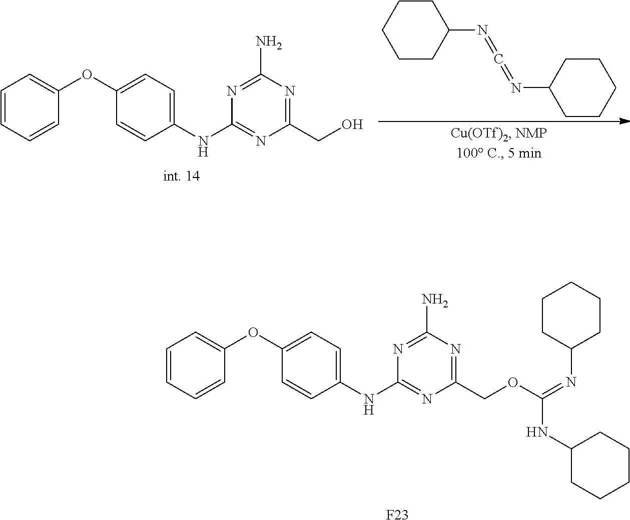 [SMILES: Nc1nc(CO)nc(Nc2ccc(Oc3ccccc3)cc2)n1 ; C(=NC1CCCCC1)=NC1CCCCC1 ; Nc1nc(CO/C(=N\C2CCCCC2)NC2CCCCC2)nc(Nc2ccc(Oc3ccccc3)cc2)n1 ; "F23"]

A mixture of {4-amino-6-[(4-phenoxyphenyl)amino]-1,3,5-triazin-2-yl}methanol (50 mg, 0.16 mmol), dicyclohexylmethanediimine (33.0 mg, 0.16 mmol) and copper(II) trifluoromethanesulfonate (1.7 mg, 0.01 mmol) was stirred at 100° C. in sealed tube for 5 min. The reaction was purified by Prep-HPLC to give title compound (20 mg). ESI-MS (EI+, m/z): 516.40. ¹H NMR (400 MHz, DMSO-d₆) δ 9.47 (s, 1H), 7.75 (d, J=8.8 Hz, 2H), 7.35 (t, J=8.0 Hz, 2H), 7.08 (t, J=7.2 Hz, 1H), 7.01-6.92 (m, 6H), 4.92 (s, 2H), 1.56 (d, J=50.1 Hz, 10H), 1.26-1.04 (m, 10H).

### Example 71. Preparation of Compounds F24 and F25

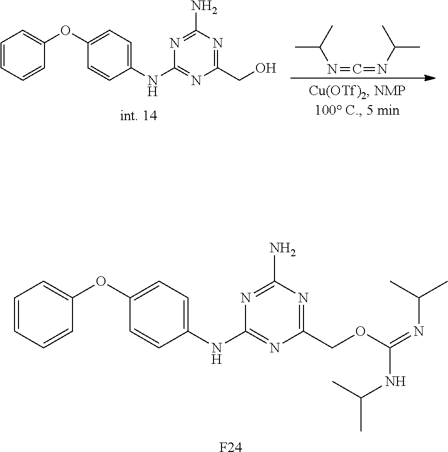

A mixture of {4-amino-6-[(4-phenoxyphenyl)amino]-1,3,5-triazin-2-yl}methanol (50 mg, 0.16 mmol), diisopropylmethanediimine (20.4 mg, 0.16 mmol) and copper(II) trifluoromethanesulfonate (1.7 mg, 0.01 mmol) was stirred at 100° C. in sealed tube for 5 min. The reaction was purified by Prep-HPLC to give title compound (20 mg). ESI-MS (EI+, m/z): 436.30. ¹H NMR (400 MHz, DMSO-d₆) δ 9.49 (s, 1H), 8.30 (s, 1H), 7.76 (d, J=8.7 Hz, 2H), 7.36 (dd, J=8.6, 7.3 Hz, 2H), 7.08 (t, J=7.3 Hz, 1H), 7.01 (s, 1H), 6.97-6.93 (m, 4H), 4.93 (s, 2H), 1.02 (d, J=22.4 Hz, 12H).

In some embodiments, F25 was synthesized as compound F24.

| Compound | Structure | LCMS, NMR |
|---|---|---|
| F25 | 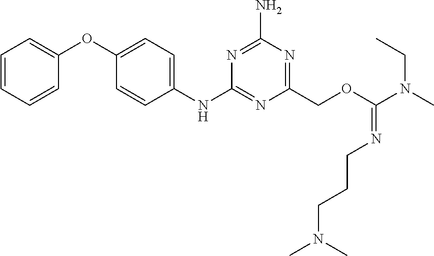 | ESI-MS (EI+, m/z): 465.30 |

### Example 72. Preparation of Compound F26

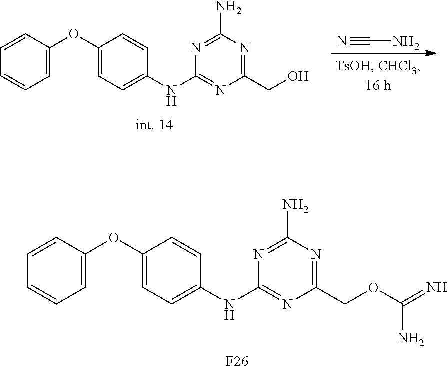

A solution of (4-amino-6-((4-phenoxyphenyl)amino)-1,3,5-triazin-2-yl)methanol (500 mg, 1.62 mmol), cyanamide (204 mg, 4.85 mmol) and TsOH (28 mg, 0.162 mmol) in CHCl₃ (5 mL) was stirred at room temperature for 16 h. The solution was purified by Prep-HPLC to give title compound (7 mg). ESI-MS (EI⁺, m/z): 352.20. ¹H NMR (400 MHz, DMSO-d(₆) δ 11.75 (s, 1H), 9.41 (s, 1H), 8.28 (s, 1H), 7.91 (d, J=8.8 Hz, 2H), 7.38 (dd, J=8.6, 7.2 Hz, 2H), 7.22 (t, J=2.8 Hz, 1H), 7.11 (t, J=7.4 Hz, 1H), 7.00 (dd, J=9.0, 7.3 Hz, 4H), 6.76 (d, J=2.9 Hz, 11H), 5.22 (d, J=5.7 Hz, 2H), 2.91 (d, J=3.0 Hz, 6H).

### Example 73. Preparation of Compound F27

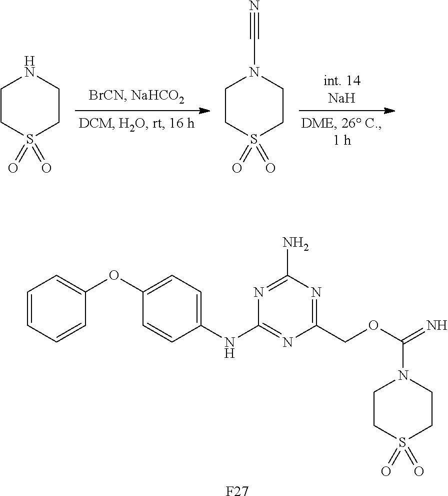 [SMILES: O=S1(=O)CCNCC1 ; N#CN1CCS(=O)(=O)CC1 ; N=C(OCc1nc(N)nc(Nc2ccc(Oc3ccccc3)cc2)n1)N1CCS(=O)(=O)CC1 ; "F27"]

1. Synthesis of thiomorpholine-4-carbonitrile 1,1-dioxide. To a stirred solution of thiomorpholine1,1-dioxide (1.0 g, 7.4 mmol) in DCM (10 mL) and water (15 mL) was added NaHCO₃ (1.86 g, 22.2 mmol) at 0° C., and then added BrCN (979 mg, 9.25 mmol). The mixture was stirred at 0° C. for 30 min, then stirred at room temperature for 16 h. The reaction mixture was filtered and the filtrate was concentrated. The crude was purified by column chromatography to afford title compound (500 mg). ¹H NMR (400 MHz, DMSO-d₆) δ 3.70-3.63 (m, 4H), 3.32-3.29 (m, 4H).

2. Synthesis of compound F27. To a stirred solution of {4-amino-6-[(4-phenoxyphenyl)amino]-1,3,5-triazin-2-yl}methanol (150 mg, 0.48 mmol) in THF (2 mL) was added NaH (24 mg, 0.96 mmol, 60%) and the mixture was stirred at 26° C. for 20 min and then 4,4-difluoropiperidine-1-carbonitrile (709 mg, 4.85 mmol) was added and stirred for another 1 h. The mixture was purified by Prep-HPLC to afford title compound (40 mg). ESI-MS (EI+, m/z): 470.30. ¹H NMR (400 MHz, DMSO-d₆) δ 9.40 (s, 1H), 7.75 (d, J=8.7 Hz, 2H), 7.39-7.33 (m, 2H), 7.32-6.93 (m, 7H), 6.82 (s, 1H), 4.91 (s, 2H), 3.80 (t, J=5.0 Hz, 4H), 3.22 (t, J=4.9 Hz, 4H).

### Example 74. Preparation of Compound F28

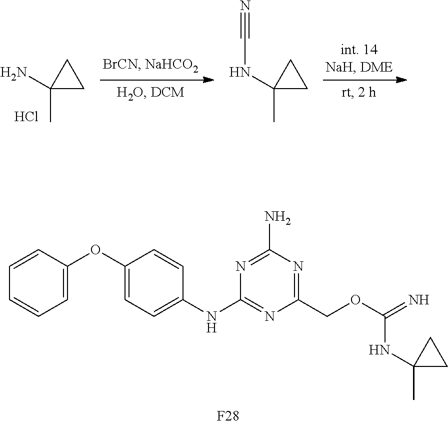

1. Synthesis of N-(1-methylcyclopropyl)cyanamide. To a solution of 1-methylcyclopropan-1-amine (1.0 g, 14.1 mmol) Sodium bicarbonate (3.54 g, 42.2 mmol) in DCM (6 mL) and H₂O (20 mL) was added BrCN (1.86 g, 17.6 mmol) in DCM (4 mL) at 0° C. The mixture was warm to room temperature and stirred for 16 h. The mixture was adjusted pH to 8-9 with a saturated aqueous NaHCO₃ solution, extracted with DCM (2×100 mL). The organic phases were dried and evaporated to afford crude title compound (800 mg). ¹³C NMR (101 MHz, DMSO-d₆) δ 116.06, 33.27, 23.01, 13.54.

2. Synthesis of compound F28. Compound F28 was synthesized as compound F27. ESI-MS (EI+, m/z): 406.25. ¹H NMR (400 MHz, DMSO-d(₆) δ 9.55 (s, 1H), 8.31 (s, 1H), 7.73 (d, J=8.4 Hz, 2H), 7.35 (t, J=7.8 Hz, 2H), 7.08 (t, J=7.4 Hz, 2H), 6.95 (dd, J=8.4, 6.0 Hz, 4H), 5.00 (s, 2H), 1.24 (s, 3H), 0.65 (s, 2H), 0.57 (d, J=4.6 Hz, 2H).

### Example 75. Preparation of Compound F29 and Other Compounds

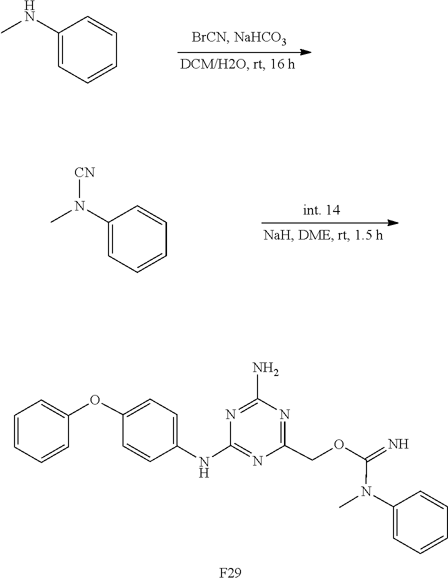

1. Synthesis of N-methyl-N-phenylcyanamide. To a solution of N-methylaniline (10.0 g, 93.3 mmol) in DCM (30 mL) and H₂O (60 mL) was added a solution of BrCN (11.9 mg, 111.98 mmol) in DCM (30 mL) dropwise at 0° C. Then the reaction was warmed to room temperature and stirred for 16 h. The reaction solution was purified by silica gel column to afford title compound (12 g). ¹H NMR (400 MHz, DMSO-d₆) δ 7.47-7.37 (m, 2H), 7.17-7.08 (m, 3H). ¹³C NMR (101 MHz, DMSO-d₆) δ 140.16, 129.43, 122.82, 114.54, 113.71, 36.24.

2. Synthesis of compound F29. Compound F29 was synthesized as compound F27. ESI-MS (EI⁺, m/z): 442.30. ¹H NMR (400 MHz, DMSO-d₆) δ 9.49 (s, 1H), 8.21 (s, 1H), 7.76 (d, J=8.6 Hz, 2H), 7.50-7.43 (m, 1H), 7.35-7.30 (m, 5H), 7.15 (t, J=7.1 Hz, 1H), 7.08 (td, J=7.5, 3.3 Hz, 2H), 6.98-6.91 (m, 5H), 4.88 (s, 2H), 3.23 (s, 3H).

Additional compounds were synthesized as compound F27, for example:

| Compound | Structure | LCMS, NMR |
|---|---|---|
| F30 | 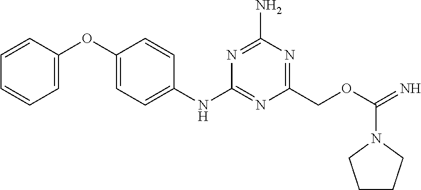 [SMILES: N=C(OCc1nc(N)nc(Nc2ccc(Oc3ccccc3)cc2)n1)N1CCCC1] | ESI-MS (EI+, m/z): 406.25. ¹H NMR (400 MHz, DMSO-d₆) δ 9.55 (s, 1H), 8.25 (s, 2H), 7.69 (d, J = 8.4 Hz, 2H), 7.36 (dd, J = 8.6, 7.3 Hz, 2H), 7.08 (t, J = 7.4 Hz, 1H), 6.97 (dd, J = 8.6, 6.8 Hz, 4H), 4.25 (s, 2H), 3.44- 3.35 (m, 4H), 1.88 (d, J = 8.1 Hz, 4H). |
| F31 | 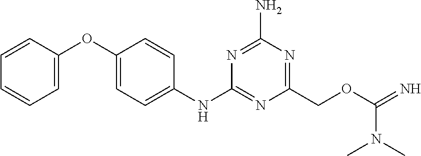 [SMILES: CN(C)C(=N)OCc1nc(N)nc(Nc2ccc(Oc3ccccc3)cc2)n1] | ESI-MS (EI+, m/z): 380.25 ¹H NMR (400 MHz, DMSO-d₆) δ 9.51 (s, 1H), 8.27 (s, 1H), 7.74 (d, J = 8.5 Hz, 2H), 7.36 (dd, J = 8.6, 7.3 Hz, 2H), 7.09 (t, J = 7.4 Hz, 3H), 6.95 (d, J = 8.9 Hz, 4H), 4.94 (s, 2H), 2.89 (s, 6H). |
| F32 | 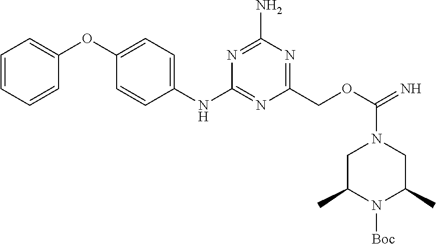 [SMILES: C[C@@H]1CN(C(=N)OCc2nc(N)nc(Nc3ccc(Oc4ccccc4)cc3)n2)C[C@H](C)N1C(=O)OC(C)(C)C] | ESI-MS(EI+, m/z) : 549.40 ¹H NMR (400 MHz, DMSO-d₆) δ 9.53 (s, 1H), 9.08 (s, 2H), 7.67 (s, 2H), 7.37 (t, J = 7.8 Hz, 2H), 7.10 (t, J = 7.5 Hz, 2H), 6.97 (t, J = 7.6 Hz, 4H), 5.27 (s, 2H), 4.19 (s,2H), 3.96 (s, 1H), 1.42 (s, 9H), 1.24 (s, 3H), 1.15 (d, J = 6.8 Hz, 6H). |
| F33 | 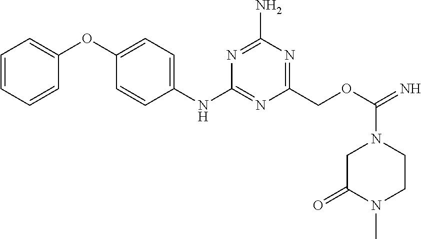 [SMILES: CN1CCN(C(=N)OCc2nc(N)nc(Nc3ccc(Oc4ccccc4)cc3)n2)CC1=O] | ESI-MS(EI+, m/z) : 449.35. |
| F34 | 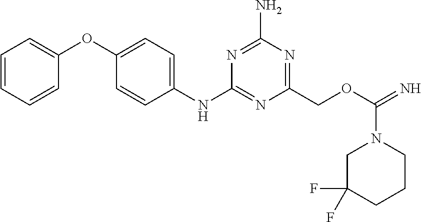 [SMILES: N=C(OCc1nc(N)nc(Nc2ccc(Oc3ccccc3)cc2)n1)N1CCCC(F)(F)C1] | ESI-MS (EI+, m/z): 479.30. ¹H NMR (400 MHz, DMSO-d₆) δ 9.51 (s, 1H), 8.18 (s, 1H), 7.77 (d, J = 9.0 Hz, 2H), 7.36 (dd, J = 8.6, 7.3 Hz, 2H), 7.08 (t, J = 7.4 Hz, 2H), 6.98-6.91 (m, 4H), 4.83 (s, 2H), 3.66 (s, 2H), 3.38 (d, J = 5.5 Hz, 2H), 1.99 (dt, J = 14.1, 7.6 Hz, 2H), 1.64 (s, 2H). |

### Example 76. Preparation of Compound F35

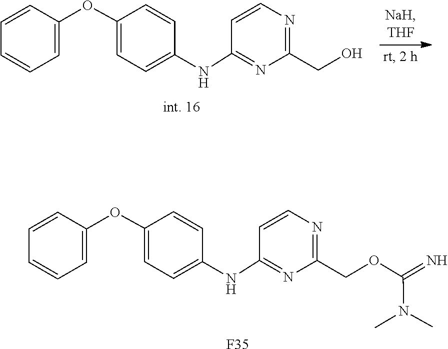 [SMILES: OCc1nccc(Nc2ccc(Oc3ccccc3)cc2)n1 ; CN(C)C(=N)OCc1nccc(Nc2ccc(Oc3ccccc3)cc2)n1 ; "F35"]

To a solution of {4-[(4-phenoxyphenyl)amino]pyrimidin-2-yl}methanol (150 mg, 0.51 mmol) in THE (1 mL) was added NaH (40.8 mg, 1.02 mmol, 60%) and stirred at 25° C. for 2 h. The mixture was concentrated in vacuo, added N,N-dimethylcyanamide (0.5 mL) and the solution was stirred at room temperature for 2 h, purified by Prep-HPLC to get the desired product (40 mg). ESI-MS (EI⁺, m/z): 364.25.

### Example 77. Preparation of compounds F36 and F37

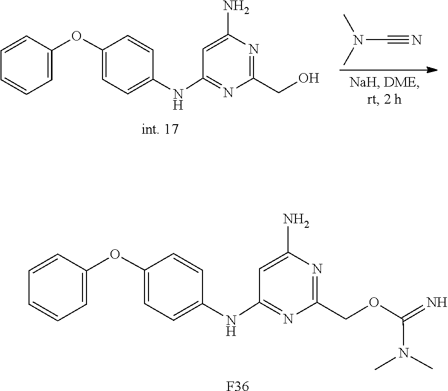

To a solution of {4-amino-6-[(4-phenoxyphenyl)amino]pyrimidin-2-yl}methanol (50 mg, 0.16 mmol) in DME (1 mL) was added NaH (16.2 mg, 0.41 mmol, 60%). The mixture was stirred at room temperature for 1 h. DME (1 mL) was removed by reduced pressure and was added into N,N-dimethylcyanamide (0.5 mL). The resulting mixture was stirred at room temperature for 1 h. The reaction was purified by Prep-HPLC to give title compound (10 mg). ESI-MS (EI+, m/z): 379.25. ¹H NMR (400 MHz, DMSO-d₆) δ 8.99 (s, 1H), 8.32 (s, 1H), 7.79 (d, J=8.6 Hz, 2H), 7.34 (t, J=7.8 Hz, 2H), 7.05 (t, J=7.4 Hz, 1H), 6.97-6.88 (m, 4H), 6.65 (s, 1H), 5.96 (s, 1H), 4.96 (s, 1H), 3.91 (s, 2H), 2.92 (s, 6H).

In some embodiments, F37 was synthesized as compound F36.

| Compound | Structure | LCMS, NMR |
|---|---|---|
| F37 | 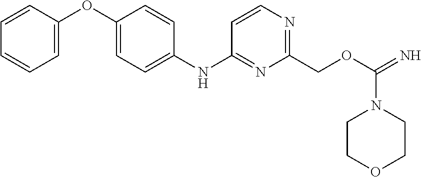 [SMILES: N=C(OCc1nccc(Nc2ccc(Oc3ccccc3)cc2)n1)N1CCOCC1] | ESI-MS (EI+, m/z): 406.25 ¹H NMR (400 MHz, DMSO-d₆) δ 9.73 (s, 1H), 8.27 (s, 1H), 8.23 (d, J = 5.9 Hz, 1H), 7.70 (d, J = 8.5 Hz, 2H), 7.37 (t, J = 7.7 Hz, 2H), 7.11 (t, J = 7.4 Hz, 1H), 6.99 (dd, J = 8.3, 5.3 Hz, 4H), 6.66 (d, J = 5.9 Hz, 1H), 5.19 (s, 2H), 3.56 (t, J = 4.8 Hz, 4H), 3.35 (t, J = 4.7 Hz, 4H). |

### Example 77. Preparation of Compound F38

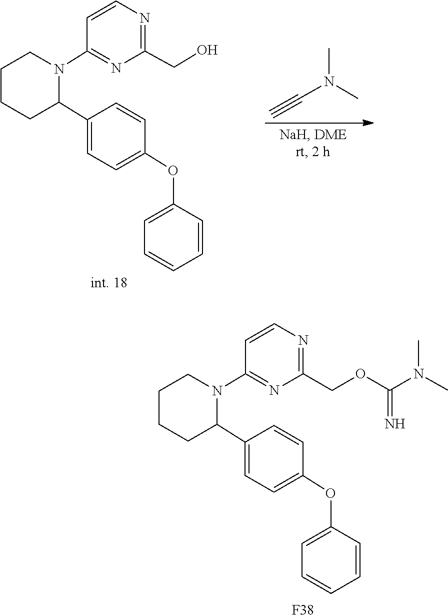

To a solution of {4-[2-(4-phenoxyphenyl)piperidin-1-yl]pyrimidin-2-yl}methanol (30 mg, 0.08 mmol) in DME (1 mL) was added NaH (2.99 mg, 0.12 mmol). The mixture was stirred at room temperature for 1 h. DME (1 mL) was removed by reduced pressure. The residue was added into N,N-dimethylcyanamide (116 mg, 1.66 mmol). The resulting mixture was stirred at room temperature for 1 h. The reaction was purified by Prep-HPLC to give title compound (10 mg). ESI-MS (EI⁺, m/z): 432.25. ¹H NMR (400 MHz, Chloroform-d) δ 8.48 (s, 1H), 8.16 (d, J=6.3 Hz, 1H), 7.33 (dd, J=8.5, 7.3 Hz, 2H), 7.12 (d, J=8.8 Hz, 3H), 7.02-6.96 (m, 4H), 6.44 (d, J=6.4 Hz, 1H), 5.29 (d, J=3.1 Hz, 2H), 4.10 (s, 1H), 3.11 (s, 6H), 3.08 (s, 2H), 2.36 (d, J=14.0 Hz, 1H), 2.00 (s, 1H), 1.65 (d, J=25.4 Hz, 4H).

### Example 78. Preparation of Compound F39

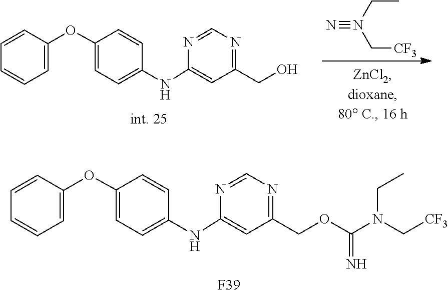

To a solution of N-ethyl-N-(2,2,2-trifluoroethyl)cyanamide (97 mg, 0.64 mmol) and (6-((4-phenoxyphenyl)amino)pyrimidin-4-yl)methanol (125 mg, 0.43 mmol) in dioxane (3 mL) was added ZnCl₂ (232 mg, 1.70 mmol) and stirred at 80° C. for 16 h under N₂ atmosphere. The reaction was filtered and concentrated. The residue was purified by Prep- HPLC to give title compound (90 mg). ESI-MS (EI⁺, m/z): 446.1 [M+H]⁺. ¹H NMR (400 MHz, DMSO-d₆) δ 9.64 (s, 1H), 8.53 (s, 1H), 7.62 (d, J=8.4 Hz, 2H), 7.37 (dd, J=7.6, 7.6 Hz, 2H), 7.11 (dd, J=7.2, 7.2 Hz, 1H), 7.03-6.97 (m, 4H), 6.71 (s, 1H), 6.09 (s, 1H), 5.01 (s, 2H), 4.14 (q, J=9.6 Hz, 2H), 3.38 (t, J=7.2 Hz, 2H), 1.08 (t, J=6.8 Hz, 3H).

### Example 79. Preparation of Compound F40

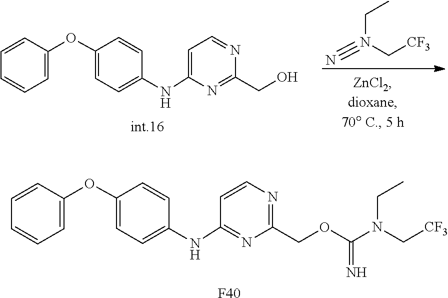

To a solution of (4-((4-phenoxyphenyl)amino)pyrimidin-2-yl)methanol (130 mg, crude) and ethyl(2,2,2-trifluoroethyl)cyanamide (202 mg, 1.33 mmol) in dioxane (3 mL) was added ZnCl₂ (242 mg, 1.77 mmol), the mixture was heated at 70° C. and stirred for 5 h. The mixture was filtered and concentrated. The residue was purified by Prep-HPLC to give title compound (95 mg). ESI-MS (EI⁺, m/z): 446.2 [M+H].

¹H NMR (400 MHz, DMSO-d₆) δ 9.62 (s, 1H), 8.22 (d, J=6.0 Hz, 1H), 7.71 (d, J=8.4 Hz, 2H), 7.41 7.33 (m, 2H), 7.14-7.06 (m, 1H), 6.98 (dd, J=8.4, 8.4 Hz, 4H), 6.63 (d, J=6.0 Hz, 1H), 5.98 5.67 (m, 1H), 5.18-4.95 (m, 2H), 4.12 (q, J=9.6 Hz, 2H), 3.37-3.29 (m, 2H), 1.03 (t, J=6.8 Hz, 3H).

### Example 80. Preparation of Compound F41

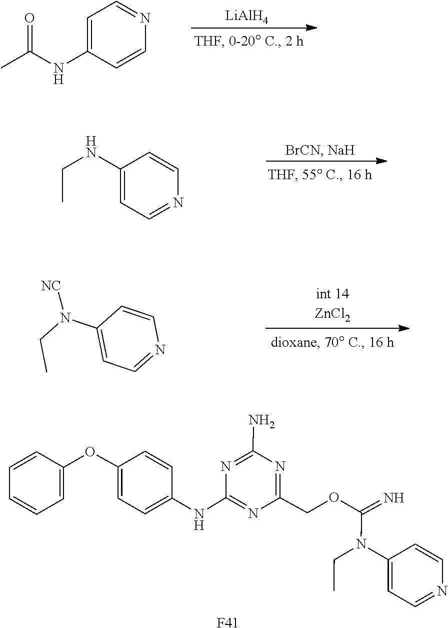

1. Synthesis of N-ethylpyridin-4-amine. To a solution of N-(pyridin-4-yl)acetamide (1.0 g, 7.3 mmol) in THF (20 mL) was added LiAlH₄ (997 mg, 29.4 mmol) at 0° C. and then stirred at 20° C. for 2 h. The mixture was cooled to 0° C., quenched by water (1 mL), followed by 1 mL of 15% aq. NaOH solution. After being stirred at 20° C. for 2 h, the solid was removed by filtration through celite pad. The filtrate was concentrated to afford title compound (897 mg crude).

2. Synthesis of N-ethyl-N-(pyridin-4-yl)cyanamide. To a solution of N-ethylpyridin-4-amine (0.97 g, 7.94 mmol) in THF (10 mL) was added NaH (953 mg, 23.8 mmol, 60% in mineral il) at 0° C. and the mixture was stirred at 20° C. for 0.5 h. Then BrCN (1.01 g, 9.53 mmol) in THF (10 mL) was added dropwise at 0° C. The reaction was stirred at 55° C. for 16 h. The reaction mixture was quenched with 50 mL of H₂O. The reaction was extracted with EtOAc (50 mL×3). The organic layers were dried, filtered and concentrated to give a residue. The residue was purified by column chromatography to afford title compound (585 mg).

3. Synthesis of compound F41. To a solution of N-ethyl-N-(pyridin-4-yl)cyanamide (95 mg, 0.65 mmol) and (4-amino-6-((4-phenoxyphenyl)amino)-1,3,5-triazin-2-yl)methanol (100 mg, 0.32 mmol) in dioxane (3 mL) was added ZnCl₂ (176 mg, 1.29 mmol). The mixture was stirred at 70° C. for 16 h under N₂ atmosphere. The reaction was concentrated to give a residue. The residue was purified by Prep-HPLC to afford title compound (108 mg). ESI-MS (EI+, m/z): 457.2 [M+H]⁺.

### Example 81. Preparation of Compound F42

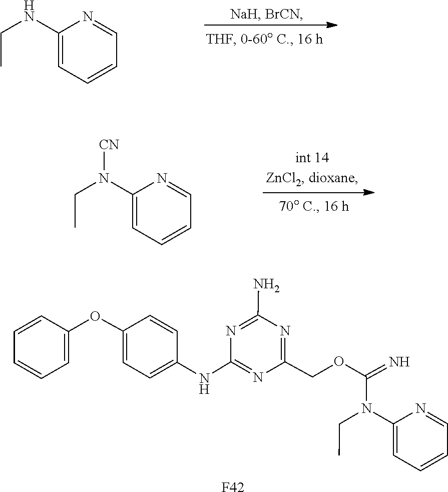

1. Synthesis of N-ethyl-N-(pyridin-2-yl)cyanamide. To a solution of N-ethylpyridin-2-amine (1.0 g, 8.19 mmol) in THF (10 mL) was added NaH (982 mg, 24.6 mmol, 60% in mineral oil) at 0° C. The mixture was stirred at 0° C. for 0.5 h, then BrCN (1.04 g, 9.82 mmol) in THF (5 mL) was added dropwise. The reaction was stirred at 60° C. for 16 h under N₂ atmosphere, quenched with 10 mL of water. The reaction solution was filtered and concentrated to give a residue. The residue was purified by column chromatography to yield title compound (340 mg). ESI-MS (EI⁺, m/z): 148.1 [M+H]⁺.

2. Synthesis of compound F42. To a solution of N-ethyl-N-(pyridin-2-yl)cyanamide (190 mg, 1.29 mmol) and (4-amino-6-((4-phenoxyphenyl)amino)-1,3,5-triazin-2-yl)methanol (100 mg, 0.32 mmol) in dioxane (3 mL) was added ZnCl₂ (176 mg, 1.29 mmol) and stirred at 70° C. for 16 h under N₂ atmosphere. The reaction was concentrated under reduce pressure to give a residue. The residue was purified by Prep-HPLC to afford title compound (120 mg). ESI-MS (EI+, m/z): 310.1 [M+H]⁺. ¹H NMR (400 MHz, CDCl₃) δ 9.69-9.49 (m, 2H), 8.53 (s, 1H), 7.97 (s, 1H), 7.80-7.52 (m, 3H), 7.52-6.74 (m, 10H), 5.33 (s, 2H), 4.15 3.75 (m, 2H), 1.30-1.08 (m, 3H).

### Example 82. Preparation of Compound F43

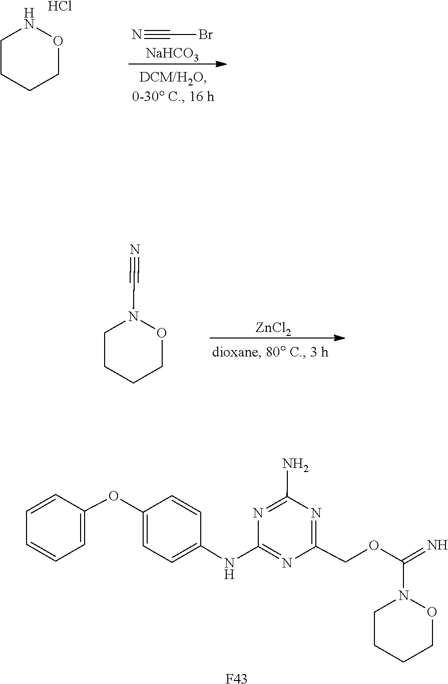

1. Synthesis of 1,2-oxazinane-2-carbonitrile. To a solution of 1,2-oxazinane hydrochloride (450 mg, 3.64 mmol) in DCM (4 mL) was added a solution of NaHCO₃ (918 mg, 10.9 mmol) in water (2 mL), the mixture was stirred at 15° C. for 10 minutes. A solution of cyanic bromide (463 mg, 4.37 mmol) in DCM (2 mL) was added dropwise to the mixture at 0° C. and the mixture was stirred at 0° C. for 1 h, then stirred at 30° C. for 16 h. The mixture was concentrated. The residue was purified by column chromatography to give title compound (257 mg).

2. Synthesis of compound F43. To a solution of 1,2-oxazinane-2-carbonitrile (200 mg, 1.78 mmol) and (4-amino-6-((4-phenoxyphenyl)amino)-1,3,5-triazin-2-yl)methanol (138 mg, 0.45 mmol) in dioxane (2 mL) was added ZnCl₂ (243 mg, 1.78 mmol). The mixture was stirred at 80° C. for 3 h. The mixture was filtered and concentrated. The residue was purified by Prep-HPLC to give title compound (130 mg). ESI-MS (EI+, m/z): 422.2 [M+H]⁺. ¹H NMR (400 MHz, DMSO-d₆) δ 9.61 (s, 1H), 9.42 (s, 1H), 7.75-7.55 (m, 2H), 7.41-7.33 (m, 2H), 7.20 (s, 1H), 7.11 (dd, J=7.2, 7.2 Hz, 1H), 7.06-6.88 (m, 5H), 5.27 (s, 2H), 4.16-4.05 (m, 2H), 3.92-3.83 (m, 2H), 1.85-1.64 ESI-MS (EI+, m/z): (m, 4H).

| Compound | Structure | LCMS, NMR |
|---|---|---|
| F44 | 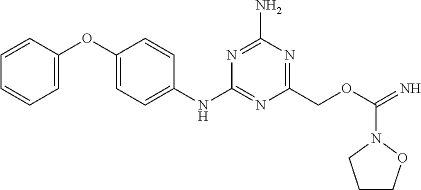 [SMILES: N=C(OCc1nc(N)nc(Nc2ccc(Oc3ccccc3)cc2)n1)N1CCCO1] | ESI-MS (EI+, m/z): 408.1 [M + H]⁺. ¹H NMR (400 MHz, DMSO-d₆) δ 9.55 (s, 1H), 7.72 (s, 2H), 7.39-7.33 (m, 3H), 7.10 (d, J = 6.4 Hz, 2H), 7.01-6.85 (m, 5H), 5.01 (s, 2H), 3.94 (t, J = 7.2 Hz, 2H), 3.67 (t, J = 7.2 Hz, 2H), 2.28-2.10 (m, 2H). |
| F45 | 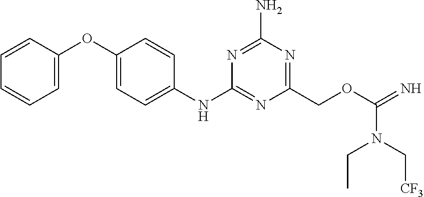 [SMILES: CCN(CC(F)(F)F)C(=N)OCc1nc(N)nc(Nc2ccc(Oc3ccccc3)cc2)n1] | ESI-MS (EI+, m/z): 462.3 [M + H]⁺. ¹H NMR (400 MHz, DMSO-d₆) δ 9.59 (s, 1H), 9.40 (s, 2H), 7.68 (s, 2H), 7.41-7.33 (m, 2H), 7.17 (s, 1H), 7.10 (t, J = 7.2 Hz, 1H), 7.05- 6.92 (m, 4H), 5.32 (s, 2H), 4.49 (s, 2H), 3.57 (s, 2H), 1.15 (s, 3H). |

### Example 83. Preparation of Compound F46

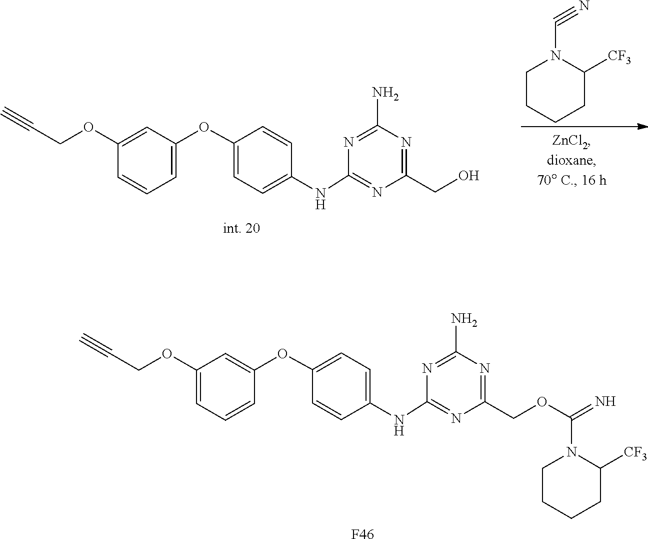 [SMILES: C#CCOc1cccc(Oc2ccc(Nc3nc(N)nc(CO)n3)cc2)c1 ; N#CN1CCCCC1C(F)(F)F ; C#CCOc1cccc(Oc2ccc(Nc3nc(N)nc(COC(=N)N4CCCCC4C(F)(F)F)n3)cc2)c1 ; "F46"]

To a solution of (4-amino-6-((4-(3-(prop-2-yn-1-yloxy)phenoxy)phenyl)amino)-1,3,5-triazin-2-yl)methanol (100 mg, 0.28 mmol) and 2-(trifluoromethyl)piperidine-1-carbonitrile (147 mg, 0.83 mmol) in 1,4-dioxane (2 mL) was added zinc(TT) chloride (38 mg, 0.28 mmol) at 25° C. The reaction was stirred at 70° C. for 16 h. The mixture was filtered and concentrated. The residue was purified by Prep-HPLC to give title compound (62 mg). ESI-MS (EI⁺, m/z): 542.1 [M+H]⁺. ¹H NMR (400 MHz, CDCl₃) δ 7.50-7.41 (m, 2H), 7.19-7.14 (m, 1H), 7.04 (s, 1H), 6.97-6.89 (m, 2H), 6.68-6.60 (m, 1H), 6.59-6.51 (m, 2H), 5.21 (s, 2H), 4.95 (d, J=14.8 Hz, 1H), 4.85-4.70 (m, 2H), 4.59 (d, J=2.4 Hz, 2H), 3.76 (d, J=12.8 Hz, 1H), 3.00 (t, J=12.8 Hz, 1H), 1.94 (d, J=13.2 Hz, 2H), 1.75-1.55 (m, 4H), 1.51-1.38 (m, 1H), 1.20-1.11 (m, 1H).

### Example 84. Preparation of Compound F47

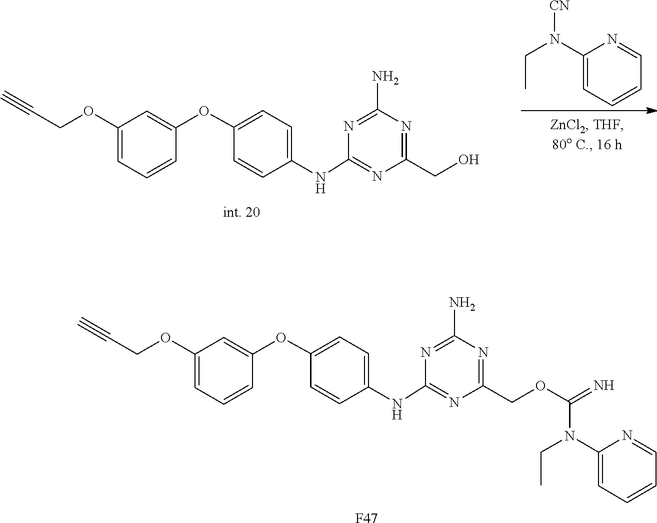 [SMILES: C#CCOc1cccc(Oc2ccc(Nc3nc(N)nc(CO)n3)cc2)c1 ; CCN(C#N)c1ccccn1 ; C#CCOc1cccc(Oc2ccc(Nc3nc(N)nc(COC(=N)N(CC)c4ccccn4)n3)cc2)c1 ; "F47"]

To a solution of N-ethyl-N-(pyridin-2-yl)cyanamide (101 mg, 0.69 mmol) and (4-amino-6-((4-(3-(prop-2-yn-1-yloxy)phenoxy)phenyl)amino)-1,3,5-triazin-2-yl)methanol (100 mg, 0.28 mmol) in THF (3 mL) was added ZnCl₂ (150 mg, 1.10 mmol) and stirred at 80° C. for 16 h under N₂ atmosphere. The reaction was concentrated under reduce pressure to give a residue. The residue was purified by Prep-HPLC to afford title compound (10 mg). ESI-MS (EI+, m/z): 511.2 [M+H]⁺. ¹H NMR (400 MHz, DMSO-d₆) δ 9.51 (s, 1H), 8.28 (s, 1H), 7.69 (d, J=35.6 Hz, 3H), 7.40 (d, J=24.4 Hz, 2H), 7.24 (dd, J=8.4, 8.4 Hz, 1H), 7.18-6.79 (m, 5H), 6.71 (dd, J=8.4, 2.4 Hz, 1H), 6.56 (s, 1H), 6.46 (s, 1H), 4.95 (s, 2H), 4.77 (d, J=2.4 Hz, 2H), 3.93 (d, J=6.8 Hz, 2H), 3.59 (t, J=2.4 Hz, 1H), 1.15 (s, 3H).

### Example 85. Preparation of Compound F48

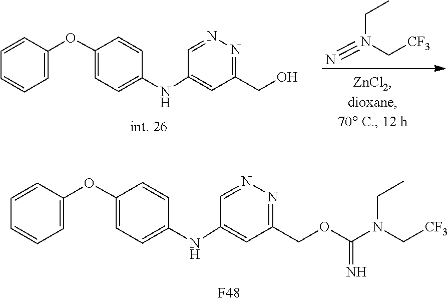

To a solution of (5-((4-phenoxyphenyl)amino)pyridazin-3-yl)methanol (20 mg, 0.07 mmol) and ethyl(2,2,2-trifluoroethyl)cyanamide (62 mg, 0.41 mmol) in dioxane (1 mL) was added ZnCl₂ (37 mg, 0.27 mmol), the mixture was heated at 70° C. for 12 h. The mixture was filtered and concentrated. The residue was purified by Prep-HPLC to give title compound (7 mg). ESI-MS (EI⁺, m/z): 446.1 [M+H]⁺. ¹H NMR (400 MHz, DMSO-d₆) δ 8.72 (d, J=2.8 Hz, 1H), 7.40-7.34 (m, 2H), 7.20-7.12 (m, 3H), 7.07-7.00 (m, 4H), 6.92-6.89 (m, 1H), 6.46 (s, 1H), 5.33 (s, 2H), 3.85 (q, J=8.8 Hz, 2H), 3.38 (q, J=7.2 Hz, 2H), 1.16 (t, J=7.2 Hz, 3H).

### Example 86. Preparation of Compound F49

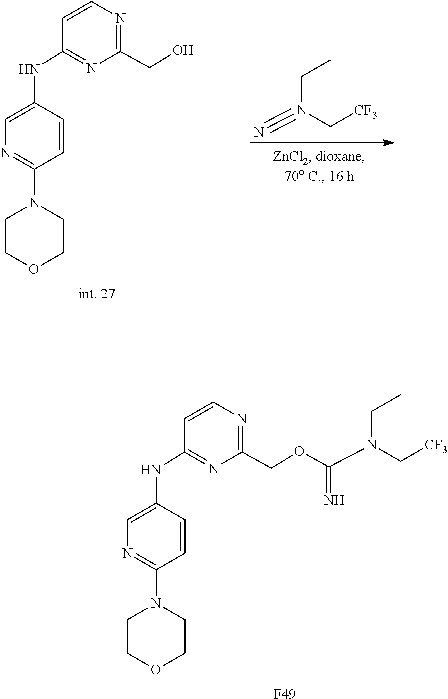

To a solution of [4-[(6-morpholino-3-pyridyl)amino]pyrimidin-2-yl]methanol (50 mg, 0.17 mmol) and ethyl (2,2,2-trifluoroethyl) cyanamide (79 mg, 0.52 mmol) was added dichlorozine (94 mg, 0.70 mmol) in 1,4-dioxane (2 mL) at 25° C. The reaction was stirred at 70° C. for 16 h. The reaction was filtered and concentrated. The residue was purified with Prep-HPLC to give title compound (27 mg). ESI-MS (EI⁺, m/z): 440.2 [M+H]⁺. ¹H NMR (400 MHz, CDCl₃) δ 8.26 (d, J=6.0 Hz, 1H), 8.21 (d, J=2.4 Hz, 1H), 7.59-7.51 (m, 1H), 6.74-6.65 (m, 1H), 6.34 (s, 1H), 5.23-5.13 (m, 2H), 4.04-3.95 (m, 2H), 3.89-3.86 (m, 4H), 3.56-3.53 (m, 4H), 3.50-3.44 (m, 2H), 1.22 (t, J=7.2 Hz, 3H).

### Example 87. Preparation of Compound F50

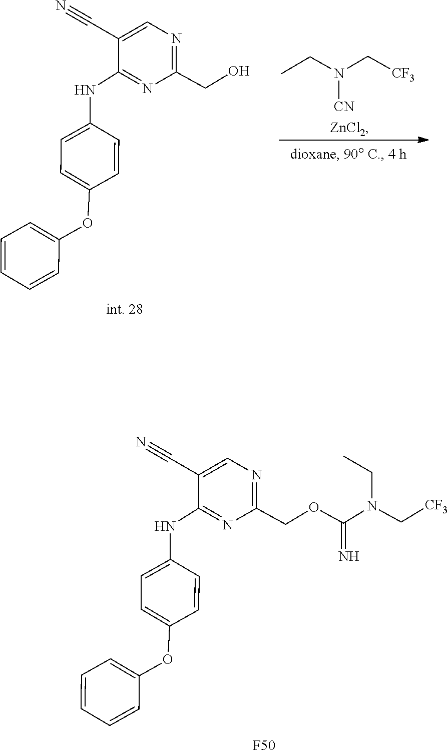

To a solution of 2-(hydroxymethyl)-4-(4-phenoxyanilino)pyrimidine-5-carbonitrile (50 mg, 0.16 mmol) and ethyl(2,2,2-trifluoroethyl)cyanamide (48 mg, 0.31 mmol) in dioxane (1 mL) was added ZnCl₂ (64 mg, 0.47 mmol) under N₂ atmosphere. The reaction was stirred at 90° C. for 4 h. The reaction was filtered and concentrated. The crude was purified by Prep-HPLC to give title compound (33 mg). ESI-MS (EI⁺, m/z): 471.3 [M+H]⁺. ¹H NMR (400 MHz, CDCl₃) (δ: 8.50 (s, 1H), 7.46-7.39 (m, 2H), 7.33-7.26 (m, 2H), 7.13 (s, 1H), 7.10-7.04 (m, 1H), 7.00-6.91 (m, 4H), 5.24 (s, 2H), 3.79 (q, J=8.8 Hz, 2H), 3.33 (q, J=7.2 Hz, 2H), 1.07 (t, J=7.2 Hz, 3H).

### Example 88. Preparation of Compound F51

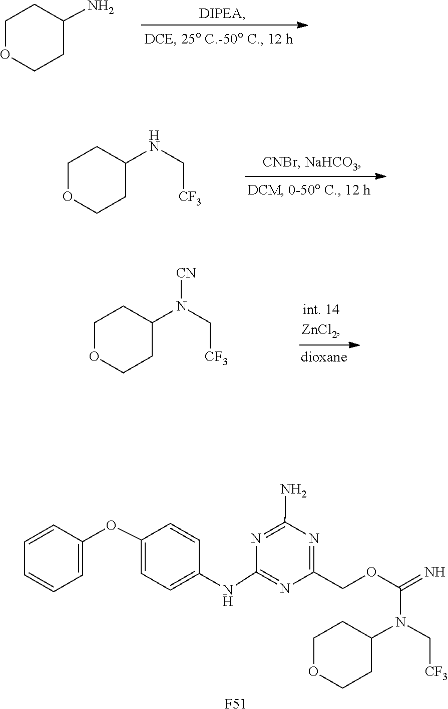 [SMILES: NC1CCOCC1 ; FC(F)(F)CNC1CCOCC1 ; N#CN(CC(F)(F)F)C1CCOCC1 ; N=C(OCc1nc(N)nc(Nc2ccc(Oc3ccccc3)cc2)n1)N(CC(F)(F)F)C1CCOCC1 ; "F51"]

1. Synthesis of compound N-(2,2,2-trifluoroethyl)tetrahydro-2H-pyran-4-amine. To a solution of tetrahydropyran-4-amine (0.2 g, 1.98 mmol) in anhydrous 1,2-dichloroethane (3 mL) was added 2,2,2-trifluoroethyl trifluoromethanesulfonate (918 mg, 3.95 mmol) and DIPEA (767 mg, 5.93 mmol) at 25° C. The mixture was stirred at 50° C. for 12 h, then was concentrated under reduced pressure to give a residue. The residue was purified by column chromatography to give title product (0.22 g). ¹H NMR (400 MHz, CDCl₃) δ 3.95-3.88 (m, 2H), 3.37-3.27 (m, 2H), 3.22-3.10 (m, 2H), 2.78-2.70 (m, 1H), 1.81 1.71 (m, 2H), 1.38-1.28 (m, 2H).

2. Synthesis of N-(tetrahydro-2H-pyran-4-yl)-N-(2,2,2-trifluoroethyl)cyanamide. To a solution of N-(2,2,2-trifluoroethyl)tetrahydropyran-4-amine (1.0 g, 5.46 mmol) in anhydrous DCM (10 mL) and saturated aqueous NaHCO₃ (8 mL) was added dropwise a solution of cyanic bromide (867 mg, 8.19 mmol) in DCM (10 mL) at 0° C. The mixture was stirred at 50° C. for 12 h, then diluted with saturated aqueous NaHCO₃ (30 mL) and extracted with DCM (40 mL×3). The combined organic phase was washed with brine (10 mL), dried over anhydrous Na₂SO₄, filtered and concentrated in vacuum to give title compound (0.8 g crude) without further purification.

3. Synthesis of compound F51. To a solution of [4-amino-6-(4-phenoxyanilino)-1,3,5-triazin-2-yl]methanol (0.2 g, 0.647 mmol) and tetrahydropyran-4-yl(2,2,2-trifluoroethyl)cyanamide (538 mg, 2.59 mmol) in anhydrous 1,4-dioxane (4 mL) was added ZnCl₂ (441 mg, 3.23 mmol) under N₂ atmosphere. The mixture was stirred at 70° C. for 12 h, then concentrated under reduced pressure. The residue was purified by Prep-HPLC to give title compound (23.5 mg). ESI-MS (EI+, m/z): 518.2 [M+H]⁺. ¹H NMR (400 MHz, DMSO-do) δ 9.48 (s, 1H), 7.73 (d, J=8.4 Hz, 2H), 7.35 (dd, J=8.4, 7.2 Hz, 2H), 7.08 (dd, J=7.2, 7.2 Hz, 2H), 6.99-6.92 (m, 4H), 4.85 (s, 2H), 4.14-4.08 (m, 2H), 3.89-3.80 (m, 2H), 3.40-3.38 (m, 2H), 3.35-3.32 (m, 1H), 1.73-1.48 (m, 4H).

### Example 89. Preparation of Compound F52

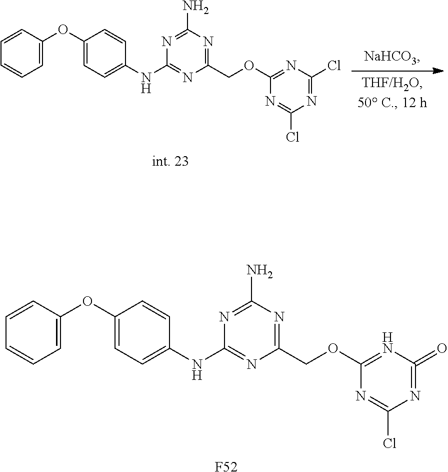

To a mixture of 6-(((4,6-dichloro-1,3,5-triazin-2-yl)oxy)methyl)-N²-(4-phenoxyphenyl)-1,3,5-triazine-2,4-diamine (100 mg, 0.22 mmol) in H₂O (1 mL) and THF (1 mL) was added NaHCO₃ (28 mg, 0.33 mmol), the mixture was stirred 50° C. for 12 h. The mixture was concentrated. The residue was purified by Prep-HPLC to give (25 mg). ESI-MS (EI+, m/z): 439.1 [M+H]⁺. ¹H NMR (400 MHz, DMSO-d₆) δ 9.59 (s, 1H), 7.75 (s, 2H), 7.39-7.31 (m, 2H), 7.28-7.14 (m, 2H), 7.12-7.04 (m, 2H), 6.99-6.90 (m, 4H), 4.99 (s, 2H).

### Example 90. Preparation of Compound F53

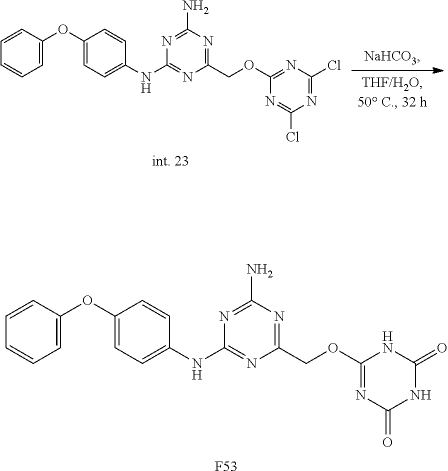 [SMILES: Nc1nc(COc2nc(Cl)nc(Cl)n2)nc(Nc2ccc(Oc3ccccc3)cc2)n1 ; Nc1nc(COc2nc(=O)[nH]c(=O)[nH]2)nc(Nc2ccc(Oc3ccccc3)cc2)n1 ; "F53"]

To a mixture of 6-(((4,6-dichloro-1,3,5-triazin-2-yl)oxy)methyl)-N2-(4-phenoxyphenyl)-1,3,5-triazine-2,4-diamine (90 mg, 0.20 mmol) in H₂O (1 mL) and THF (1 mL) was added NaHCO₃ (50 mg, 0.59 mmol), the mixture was stirred at 50° C. for 32 h. The reaction was concentrated. The residue was purified by Prep-HPLC to give title product (6.6 mg). ESI-MS (EI+, m/z): 421.1.

### Example 91. Preparation of Compound F54

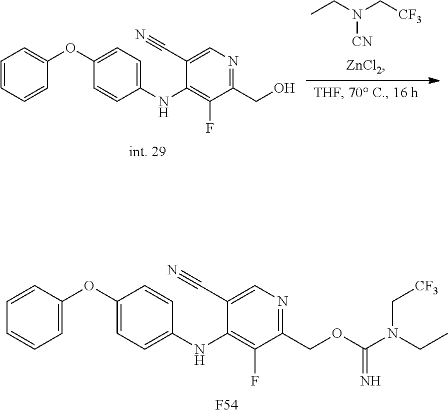

To a solution of 5-fluoro-6-(hydroxymethyl)-4-(4-phenoxyanilino)pyridine-3-carbonitrile (50 mg, 0.15 mmol) and ethyl(2,2,2-trifluoroethyl)cyanamide (68 mg, 0.45 mmol) in THF (5 mL) was added ZnCl₂ (81 mg, 0.6 mmol). The mixture was stirred at 70° C. for 16 h under N₂ atmosphere. The solvent was removed to yield a residue which was purified by Prep-HPLC to give title compound (36.4 mg). ESI-MS (EI⁺, m/z): 488.1 [M+H]⁺. ¹H NMR (400 MHz, CDCl₃) δ 8.32 (s, 1H), 7.40-7.32 (m, 2H), 7.24-7.18 (m, 2H), 7.17-7.09 (m, 1H), 7.09-7.01 (m, 4H), 6.64 (d, J=4.0 Hz, 1H), 5.29 (d, J=2.4 Hz, 2H), 3.86 (q, J=8.8 Hz, 2H), 3.37 (q, J=7.2 Hz, 2H), 1.17 (t, J=7.2 Hz, 3H).

### Example 92. Preparation of Compound E15

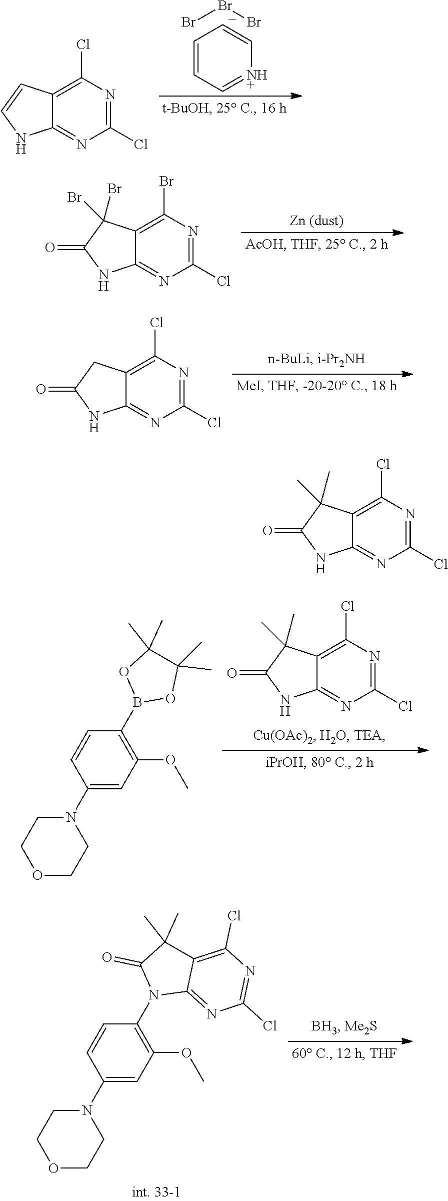

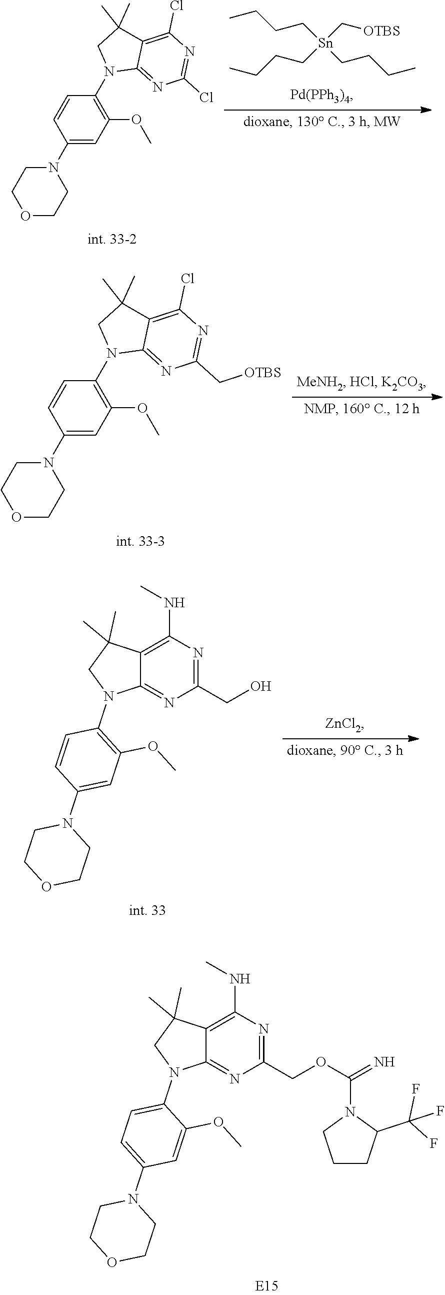

1. Synthesis of compound 4,5,5-tribromo-2-chloro-5,7-dihydro-6H-pyrrolo[2,3-d]pyrimidin-6-one. To a solution of 2,4-dichloro-7H-pyrrolo[2,3-d]pyrimidine (20.0 g, 106 mmol) in t-BuOH (400 mL) was added pyridinium tribromide (204 g, 638 mmol) portionwise at 25° C. The mixture was stirred at 25° C. for 16 h. H₂O was added to the reaction and extracted with EtOAc, washed with brine, and dried with Na₂SO₄. The organic layers were concentrated to afford title compound (45.0 g). LCMS (ESI): m/z, 361.8, 363.8 [M+H]⁺.

2. Synthesis of compound 2,4-dichloro-5,7-dihydropyrrolo[2,3-d]pyrimidin-6-one. To a solution of 4,5,5-tribromo-2-chloro-5,7-dihydro-6H-pyrrolo[2,3-d]pyrimidin-6-one (35.0 g, 96.7 mmol) in THF (150 mL) was added AcOH (150 mL) at 0° C., then Zn dust (25.3 g, 387 mmol) was added. The mixture was stirred at 0° C. for 30 min, then warmed up to 25° C. for 2 h. The mixture was filtered, the filtrate was concentrated, and the residue was purified by column chromatography to afford title compound (18.0 g). ¹H NMR (400 MHz, DMSO-d₆) δ 12.01 (s, 1H), 3.63 (s, 2H). LCMS (ESI): m/z, 203.9 205.9 [M+H]⁺.

3. Synthesis of compound 2,4-dichloro-5,5-dimethyl-7H-pyrrolo[2,3-d]pyrimidin-6-one. To a stirred solution of 2,4-dichloro-5,7-dihydropyrrolo[2,3-d]pyrimidin-6-one (1.0 g, 4.90 mmol) in dry THF (22.2 mL) at -20° C. was added i-Pr₂NH (1.49 g, 14.70 mmol), followed by n-BuLi (2.5 M, 7.84 mL) for 1 h. The reaction mixture was allowed to warm to 0° C. and then Mel (0.92 mL) was added. The reaction mixture was stirred at 20° C. for 18 h. The reaction mixture was cooled to 0° C., quenched with sat. NH₄Cl and extracted with EtOAc. The combined organic layers were concentrated to give the residue which was purified by column chromatography to afford title compound. ¹H NMR (400 MHz, CDCl₃) δ 8.58 (s, 1H), 1.54 (s, 6H).

4. Synthesis of compound int. 33-1. A mixture of 2,4-dichloro-5,5-dimethyl-7H-pyrrolo[2,3-d]pyrimidin-6-one (3.0 g, 12.9 mmol), 4-[3-methoxy-4-(4,4,5,5-tetramethyl-1,3,2-dioxaborolan-2-yl)phenyl]morpholine (6.19 g, 19.4 mmol), Cu(OAc)₂·H₂O (2.58 g, 12.9 mmol), (2R,3S)-2-[[(2R)-2-acetamido-3-(3-fluorophenyl)propanoyl]amino]-5-methoxy-4-methoxycarbonyl-3-methyl-5-oxo-pentanoic acid (11.4 g, 25.8 mmol), and TEA (3.59 mL) in i-PrOH (50 mL) was stirred at 80° C. for 2 h under O₂ atmosphere. The mixture was filtered and concentrated. The residue was purified by column chromatography to afford title compound (1.00 g). LCMS (ESI): m/z, 423.2, 425.2 [M+H]⁺.

5. Synthesis of compound int. 33-2. To a solution of 2,4-dichloro-7-(2-methoxy-4-morpholinophenyl)-5,5-dimethyl-5,7-dihydro-6H-pyrrolo[2,3-d]pyrimidin-6-one (1.50 g, 3.54 mmol) in THF (15 mL) was added BH₃.Me₂S (2 M, 8.86 mL) dropwise. The mixture was stirred at 60° C. for 12 h. The mixture was quenched by sat. aq. NH₄Cl and concentrated. The residue was purified by column chromatography to afford title compound (1.0 g). LCMS (ESI): m/z, 409.2, 411.2 [M+H]⁺.

6. Synthesis of compound int. 33-3. A mixture of 4-(4-(2,4-dichloro-5,5-dimethyl-5,6-dihydro-7H-pyrrolo[2,3-d]pyrimidin-7-yl)-3-methoxyphenyl)morpholine (1.0 g, 2.44 mmol), tert-butyl-dimethyl-(tributylstannylmethoxy)silane (1.38 g, 3.18 mmol), and Pd(PPh₃)₄ (338 mg, 0.293 mmol) in dioxane (15 mL) was stirred at 130° C. for 3 h under N₂ atmosphere by MW. The mixture was concentrated. The residue was purified by column chromatography to afford title compound (300 mg). LCMS (ESI): m/z, 519.3 [M+H]⁺.

7. Synthesis of compound int. 33. A mixture of 4-(4-(2-(((tert-butyldimethylsilyl)oxy)methyl)-4-chloro-5,5-dimethyl-5,6-dihydro-7H-pyrrolo[2,3-d]pyrimidin-7-yl)-3-methoxyphenyl)morpholine (280 mg, 0.539 mmol), K₂CO₃ (mg, mmol), methanamine hydrochloride (182 mg, 2.70 mmol) in NMP (10 mL) was stirred at 160° C. for 12 h. The mixture was diluted with EA, washed with sat. aq. NaCl, dried over Na₂SO₄, filtered, and concentrated. The residue was purified by column chromatography to afford title compound (150 mg). LCMS (ESI): m/z, 400.3 [M+H]⁺.

8. Synthesis of compound E15. To a solution of (7-(2-methoxy-4-morpholinophenyl)-5,5-dimethyl-4-(methylamino)-6,7-dihydro-5H-pyrrolo[2,3-d]pyrimidin-2-yl)methanol (205 mg, 0.513 mmol), 2-(trifluoromethyl)pyrrolidine-1-carbonitrile (252 mg, 1.54 mmol) in dioxane (10 mL) was added ZnCl₂ (209 mg, 1.54 mmol). The mixture was stirred at 90° C. for 3 h. The mixture was concentrated. The residue was purified by pre-HPLC to afford title compound (101 mg). ¹H NMR (400 MHz, CDCl₃) δ 7.25 (d, J=8.0 Hz, 1H), 6.49-6.47 (m, 2H), 5.08-4.87 (m, 2H), 4.58-4.54 (m, 1H), 4.15-4.12 (m, 1H), 3.87 (t, J=4.8 Hz, 4H), 3.78 (s, 3H), 3.58-3.52 (m, 2H), 3.48-3.44 (m, 2H), 3.15 (t, J=5.2 Hz, 4H), 3.03 (d, J=4.8 Hz, 3H), 2.09-2.00 (m, 2H), 1.92-1.83 (m, 2H), 1.38 (s, 6H). LCMS (ESI): m/z, 564.3 [M+H]⁺.

### Example 93. Preparation of Compound E16

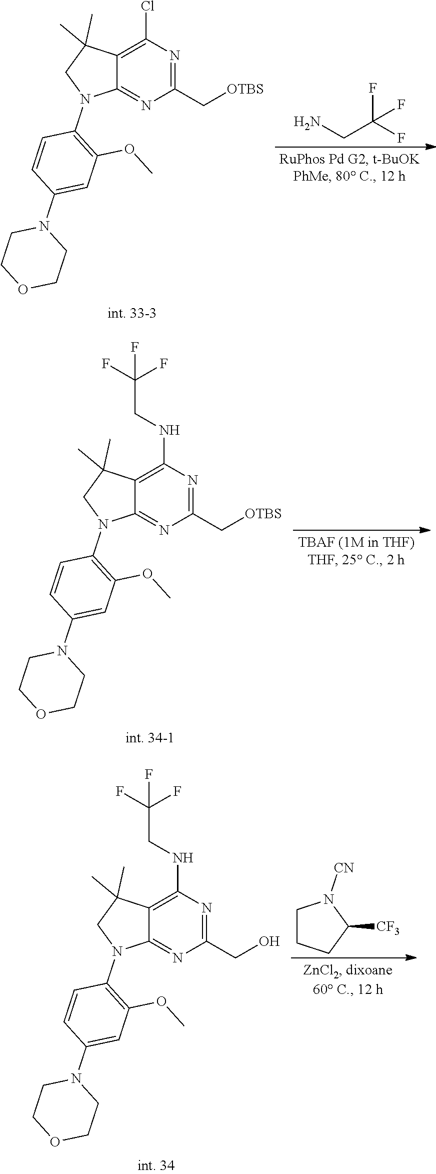

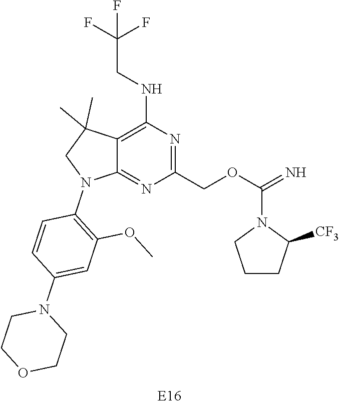 [SMILES: COc1cc(N2CCOCC2)ccc1N1CC(C)(C)c2c(NCC(F)(F)F)nc(COC(=N)N3CCC[C@@H]3C(F)(F)F)nc21]

1. Synthesis of compound int. 34-1. A mixture of 4-(4-(2-(((tert-butyldimethylsilyl)oxy)methyl)-4-chloro-5,5-dimethyl-5,6-dihydro-7H-pyrrolo[2,3-d]pyrimidin-7-yl)-3-methoxyphenyl)morpholine (600 mg, 1.16 mmol), 2,2,2-trifluoroethylamine (572 mg, 461 μL, 5.78 mmol), t-BuOK (259 mg, 2.31 mmol), and RuPhos Pd G2 (134 mg, 0.173 mmol) in toluene (10 mL) was stirred at 80° C. for 12 h under N₂ atmosphere. The mixture was filtered. The filtrate was concentrated to afford title compound (600 mg crude). LCMS (ESI): m/z, 519.3 [M+H]⁺.

2. Synthesis of compound int. 34. To a solution of 2-(((tert-butyldimethylsilyl)oxy)methyl)-7-(2-methoxy-4-morpholinophenyl)-5,5-dimethyl-N-(2,2,2-trifluoroethyl)-6,7-dihydro-5H-pyrrolo[2,3-d]pyrimidin-4-amine (600 mg, 1.03 mmol) in THF (20 mL) was added TBAF (3.09 mL, 1 M in THF). The mixture was stirred at 25° C. for 2 h. The mixture was concentrated. The residue was purified by column chromatography to afford title compound (410 mg). LCMS (ESI): m/z, 468.2 [M+H]⁺.

3. Synthesis of compound E16. To a solution of (7-(2-methoxy-4-morpholinophenyl)-5,5-dimethyl-4-((2,2,2-trifluoroethyl)amino)-6,7-dihydro-5H-pyrrolo[2,3-d]pyrimidin-2-yl)methanol (120 mg, 0.256 mmol), (2R)-2-(trifluoromethyl)pyrrolidine-1-carbonitrile (126 mg, 0.770 mmol) in 1,4-dioxane (3 mL) was added ZnCl₂(70 mg, 0.513 mmol). The mixture was stirred at 60° C. for 12 h under N₂ atmosphere. The mixture was concentrated. The residue was purified by pre-HPLC to afford title compound (100 mg). ¹H NMR (400 MHz, CDCl₃) δ 7.25-7.23 (m, 1H), 6.49-6.47 (m, 2H), 5.07-4.88 (m, 2H), 4.55-4.48 (m, 1H), 4.32-4.21 (m, 3H), 3.86 (t, J=5.2 Hz, 4H), 3.78 (s, 3H), 3.61-3.56 (m, 2H), 3.51-3.43 (m, 2H), 3.16 (t, J=5.2 Hz, 4H), 2.10-2.02 (m, 2H), 1.91-1.85 (m, 2H), 1.400 (s, 6H). LCMS (ESI): m/z, 632.4 [M+H]⁺.

### Example 94. Preparation of Compound E17

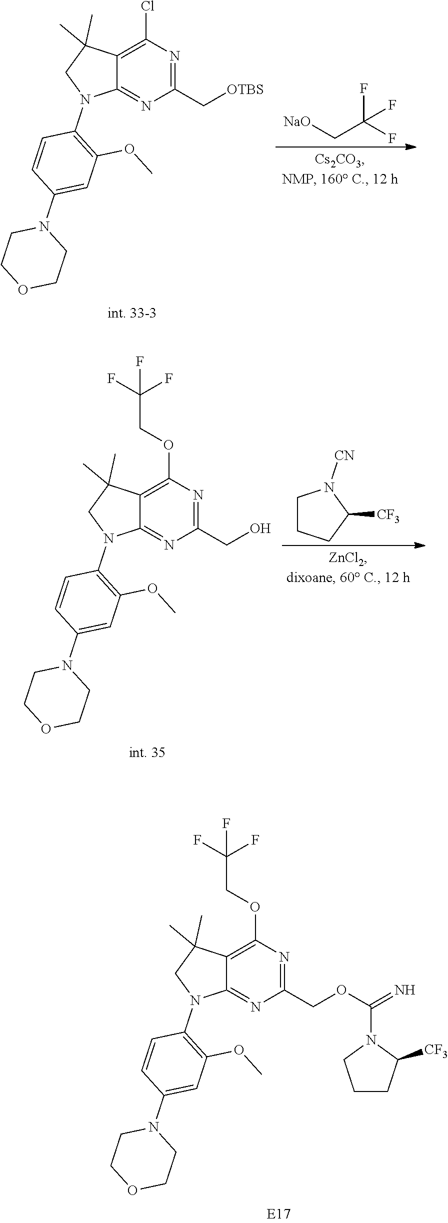

1. Synthesis of compound int. 35. To a solution of 4-(4-(2-(((tert-butyldimethylsilyl)oxy)methyl)-4-chloro-5,5-dimethyl-5,6-dihydro-7H-pyrrolo[2,3-d]pyrimidin-7-yl)-3-methoxyphenyl)morpholine (180 mg, 0.346 mmol), (2,2,2-trifluoroethoxy)sodium (211 mg, 1.73 mmol), and NMP (3 mL) was added Cs₂CO₃ (564 mg, 1.73 mmol). The mixture was stirred at 160° C. for 12 h. The mixture was diluted with EA (10 mL), washed with sat. aq. NaCl, dried over Na₂SO₄, filtered and concentrated. The residue was purified by column chromatography to afford title compound (93 mg). LCMS (ESI): m/z, 469.3 [M+H]⁺.

2. Synthesis of compound E17. Compound E17 was synthesized as compound E16. ¹H NMR (400 MHz, CDCl₃) δ 7.23-7.21 (m, 1H), 6.49-6.46 (m, 2H), 5.11-4.92 (m, 2H), 4.80-4.73 (m, 2H), 4.51-4.43 (m, 1H), 3.87 (t, J=5.2 Hz, 4H), 3.86 (s, 3H), 3.61-3.56 (m, 2H), 3.51-3.43 (m, 2H), 3.16 (t, J=5.2 Hz, 4H), 2.10-2.02 (m, 2H), 1.91-1.85 (m, 2H), 1.400 (s, 6H). LCMS (ESI): m/z, 633.4 [M+H]⁺.

### Example 95. Preparation of Compound E18

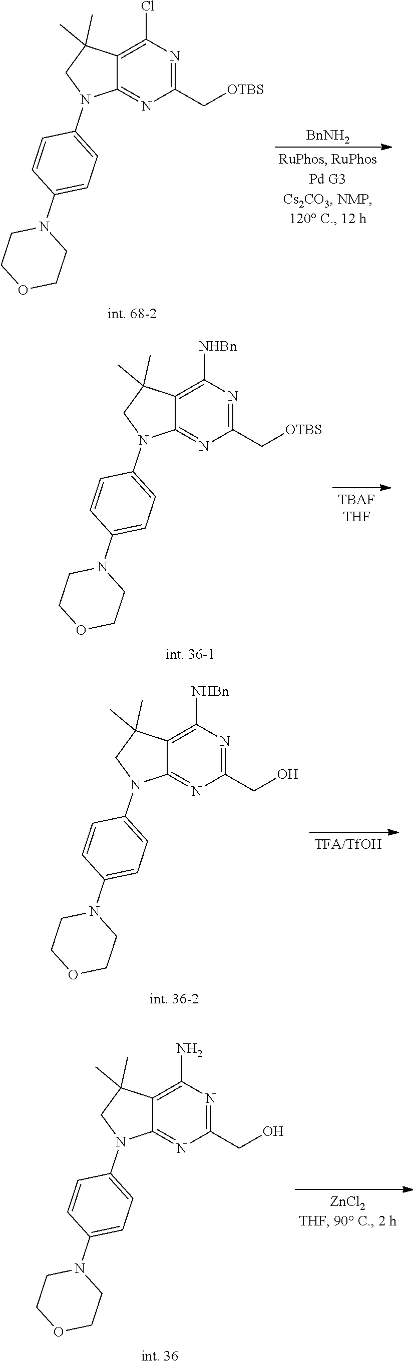

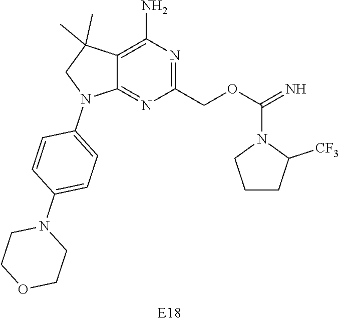 [SMILES: CC1(C)CN(c2ccc(N3CCOCC3)cc2)c2nc(COC(=N)N3CCCC3C(F)(F)F)nc(N)c21]

1. Synthesis of compound int. 36-1. A mixture of 4-(4-(2-(((tert-butyldimethylsilyl)oxy)methyl)-4-chloro-5,5-dimethyl-5,6-dihydro-7H-pyrrolo[2,3-d]pyrimidin-7-yl)phenyl)morpholine (0.50 g, 1.02 mmol), phenylmethanamine (0.22 mL), RuPhos Pd G3 (170 mg, 0.21 mmol), RuPhos (95 mg, 0.20 mmol) and Cs₂CO₃ (666 mg, 2.04 mmol) in NMP (10 mL) was stirred at 120° C. for 12 h under N₂ atmosphere. The reaction mixture was diluted with EtOAc (40 mL), washed with sat. aq. NH₄Cl and brine. The combined organic phase was dried over Na₂SO₄, filtered and concentrated to give title compound (0.7 g crude).

2. Synthesis of compound int. 36-2. A mixture of N-benzyl-2-(((tert-butyldimethylsilyl)oxy)methyl)-5,5-dimethyl-7-(4-morpholinophenyl)-6,7-dihydro-5H-pyrrolo[2,3-d]pyrimidin-4-amine (0.7 g, 1.25 mmol) and TBAF (3.75 mL, 1M) in THF (8 mL) was stirred at 25° C. for 2 h under N₂ atmosphere. The reaction mixture was diluted with EtOAc (40 mL), washed with sat. aq. NH₄Cl and brine. The organic phase was dried over Na₂SO₄, filtered and concentrated. The residue was purified by column chromatography to give title compound (0.40 g).

3. Synthesis of compound int. 36. (4-(benzylamino)-5,5-dimethyl-7-(4-morpholinophenyl)-6,7-dihydro-5H-pyrrolo[2,3-d]pyrimidin-2-yl)methanol (700 mg, 1.57 mmol) was added to TFA (15 mL) and TfOH (3 mL) at 0° C. and stirred at 25° C. for 16 h. The solvent was removed and the residue was treated with sat. aq. NaHCO₃ to adjusted pH to 8. Water (20 mL) was added and extracted with EtOAc. The combined organic phases were washed with brine (20 mL), dried over Na₂SO₄, filtered and concentrated to give title compound (500 mg) which was used to next step directly.

4. Synthesis of compound E18. Compound E18 was synthesized as compound E15. ¹H NMR (400 MHz, CDCl₃) δ 7.57 (d, J=8.4 Hz, 2H), 7.02-6.81 (m, 2H), 5.23-5.00 (m, 2H), 4.89 (s, 2H), 4.59 (s, 2H), 3.96-3.81 (m, 4H), 3.61 (d, J=10.0 Hz, 2H), 3.20-3.04 (m, 4H), 2.17 (d, J=6.4 Hz, 2H), 1.42 (s, 6H). LCMS (ESI): m/z, 520.3 [M+H]⁺.

### Example 96. Preparation of Compound E19

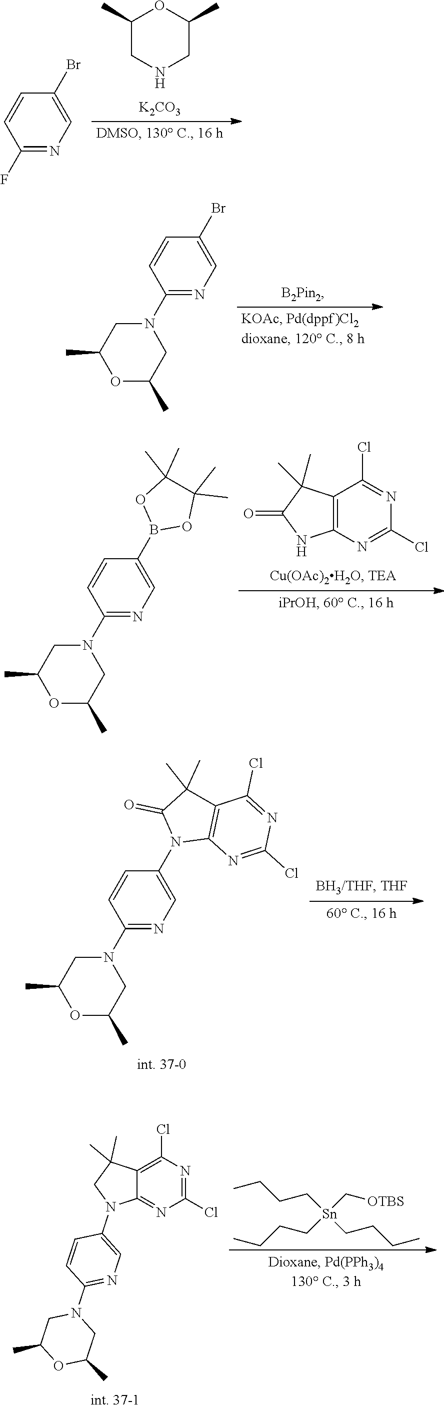

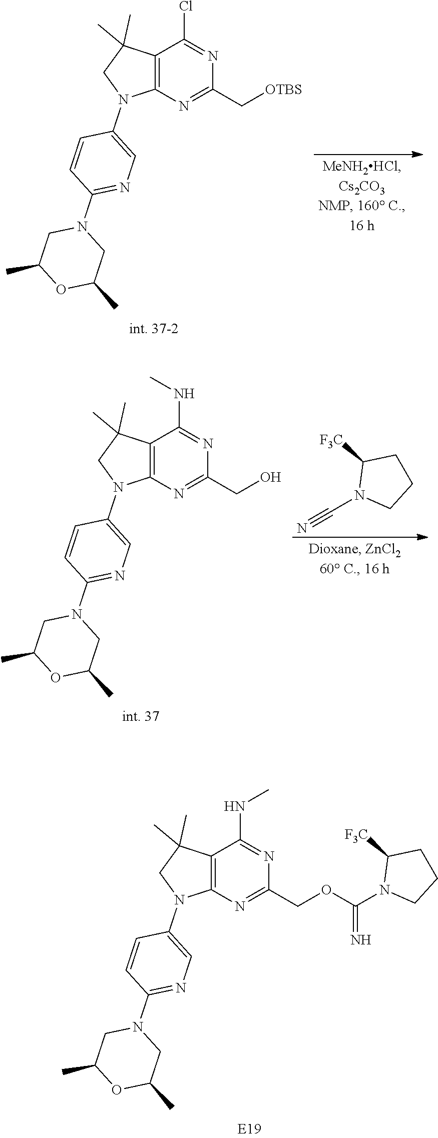 [SMILES: C[C@@H]1CN(c2ccc(N3CC(C)(C)c4c(Cl)nc(CO[Si](C)(C)C(C)(C)C)nc43)cn2)C[C@H](C)O1 ; CNc1nc(CO)nc2c1C(C)(C)CN2c1ccc(N2C[C@@H](C)O[C@@H](C)C2)nc1 ; N#CN1CCC[C@@H]1C(F)(F)F ; CNc1nc(COC(=N)N2CCC[C@@H]2C(F)(F)F)nc2c1C(C)(C)CN2c1ccc(N2C[C@@H](C)O[C@@H](C)C2)nc1]

1. Synthesis of compound int. 37-1. To a solution of (2R,6S)-2,6-dimethylmorpholine (13.09 g, 113.65 mmol) and 5-bromo-2-fluoro-pyridine (10.0 g, 56.8 mmol) in DMSO (30 mL) was added K2CO3 (23.6 g, 170. mmol) at 25° C. The reaction was stirred at 130° C. for 16 h. The reaction was filtered and the filtrate was diluted with water, extracted with EA. The organic phases were washed with brine, dried with Na2SO4, filtered and concentrated. The mixture was purified with column chromatography to give title compound (14.0 g). LCMS (ESI): m/z, 271.1, 273.1 [M+H]+.

2. Synthesis of compound int. 37-1. To a solution of (2R,6S)-4-(5-bromo-2-pyridyl)-2,6-dimethyl-morpholine (7.0 g, 25.8 mmol) and 4,4,5,5-tetramethyl-2-(4,4,5,5-tetramethyl-1,3,2-dioxaborolan-2-yl)-1,3,2-dioxaborolane (9.83 g. 38.7 mmol) in 1,4-dioxane (40 mL) was added Pd(dppf)Cl2 (1.89 g, 2.58 mmol) and potassium acetate (5.07 g, 51.6 mmol) at 25° C. The reaction was stirred at 120° C. for 8 h. The reaction was filtered and the filtrate was concentrated. The mixture was purified with column chromatography to give title compound (5.1 g). LCMS (ESI): m/z, 237.2[M+H]+. 1H NMR (400 MHz, DMSO-d6) δ 8.32 (dd, J=2.0, 0.8 Hz, 1H), 7.70 (dd, J=8.8, 2.0 Hz, 1H), 6.80 (dd, J=8.8, 0.8 Hz, 1H), 4.26-4.17 (m, 2H), 3.57 (m, 2H), 2.41 (m, 2H), 1.16 (s, 12H), 1.07 (s, 6H).

3. Synthesis of compound int. 37-0. To a solution of 2,4-dichloro-5,5-dimethyl-7H-pyrrolo[2,3-d]pyrimidin-6-one (255 mg, 1.10 mmol) and (2S,6R)-2,6-dimethyl-4-[5-(4,4,5,5-tetramethyl-1,3,2-dioxaborolan-2-yl)-2-pyridyl]morpholine (700 mg, 2.20 mmol) in propan-2-ol (10 mL) was added 4A MS, TEA (0.61 mL) and copper diacetate hydrate (263 mg, 1.32 mmol) at 25° C. The reaction was stirred at 60° C. for 16 h under 02. The mixture was filtered and the filtrate was concentrated. The residue was purified by column chromatography to give title compound (400 mg). LCMS (ESI): m/z, 237.2 [M+H]+.

4. Synthesis of compound int. 37-1. To a solution of 2,4-dichloro-7-(6-((2R,6S)-2,6-dimethylmorpholino)pyridin-3-yl)-5,5-dimethyl-5,7-dihydro-6H-pyrrolo[2,3-d]pyrimidin-6-one (1.00 g, 2.37 mmol) in THF (10 mL) was added borane-methyl sulfide complex (0.71 mL, 10 M in DMS). The reaction was stirred at 60° C. for 16 h. The reaction mixture was cooled to 0° C., then quenched by 5 mL of MeOH. The mixture was stirred at room temperature for 2 h, then concentrated. The residue was purified by column chromatography to give title compound (570 mg). ¹H NMR (400 MHz, CDCl₃) δ 8.30 (d, J=2.8 Hz, 1H), 8.01 (dd, J=9.2, 2.9 Hz, 1H), 6.69 (d, J=9.2 Hz, 1H), 4.01 (dd, J=2.0, 13.2 Hz, 2H), 3.81 (s, 2H), 3.75-3.69 (m, 2H), 2.55-2.50 (m, 2H), 1.52 (s, 6H), 1.27 (d, J=6.4 Hz, 6H). LCMS (ESI): m/z, 408.1 [M+H]⁺.

5. Synthesis of compound int. 37-2. To a solution of (2R,6S)-4-(5-(2,4-dichloro-5,5-dimethyl-5,6-dihydro-7H-pyrrolo[2,3-d]pyrimidin-7-yl)pyridin-2-yl)-2,6-dimethylmorpholine (470 mg, 1.15 mmol) in 1,4-dioxane (6 mL) was added Pd(PPh₃)₄ (266 mg, 0.23 mmol) and tert-butyl-dimethyl-(tributylstannylmethoxy)silane (1.00 g, 2.30 mmol). The reaction was stirred at 130° C. for 3 h under N₂ atmosphere. The mixture was diluted with water (20 mL) and extracted with EtOAc, the organic layer was washed with brine, dried over Na₂SO₄, filtered and concentrated. The residue was purified by column chromatography to give title compound (480 mg). LCMS (ESI): m/z, 518.4 [M+H]⁺.

6. Synthesis of compound int. 37. To a solution of (2R,6S)-4-(5-(2-(((tert-butyldimethylsilyl)oxy)methyl)-4-chloro-5,5-dimethyl-5,6-dihydro-7H-pyrrolo[2,3-d]pyrimidin-7-yl)pyridin-2-yl)-2,6-dimethylmorpholine (240 mg, 463.17 μmol) in NMP (6 mL) was added CH₃NH₂—HCl (250 mg, 3.71 mmol) and Cs₂CO₃ (1.21 g, 3.71 mmol). The reaction was stirred at 160° C. for 16 h. The mixture was diluted with water (10 mL) and extracted with EtOAc, the organic layer was washed with brine, dried over Na₂SO₄, filtered and concentrated. The residue was purified by column chromatography to give title compound (40 mg). ¹H NMR (400 MHz, CDCl₃) δ 8.30 (d, J=2.8 Hz, 1H), 8.04 (d, J=9.2 Hz, 1H), 6.68 (d, J=9.2 Hz, 1H), 4.51 (d, J=4.4 Hz, 2H), 4.25 (d, J=5.2 Hz, 1H), 3.97 (s, 2H), 3.94 (s, 1H), 3.77-3.73 (m, 2H), 3.66 (s, 2H), 3.07 (d, J=4.8 Hz, 3H), 2.53-2.46 (m, 2H), 1.42 (s, 6H), 1.27 (d, J=6.4 Hz, 6H). LCMS (ESI): m/z, 399.3 [M+H]⁺.

7. Synthesis of compound E19. Compound E19 was synthesized as compound E16. ¹H NMR (400 MHz, CDCl₃) δ 8.31 (d, J=2.8 Hz, 1H), 8.03 (dd, J=2.8, 9.2 Hz, 1H), 6.67 (d, J=9.2 Hz, 1H), 5.18 (d, J=14.0 Hz, 1H), 5.03 (d, J=14.0 Hz, 1H), 4.64 (t, J=7.6 Hz, 1H), 4.24 (d, J=5.2 Hz, 1H), 3.95 (dd, J=2.4, 12.8 Hz, 2H), 3.77-3.72 (m, 2H), 3.63 (s, 2H), 3.04 (d, J=4.8 Hz, 3H), 2.51-2.45 (m, 2H), 2.16-1.98 (m, 5H), 1.40 (s, 6H), 1.27 (d, J=6.4 Hz, 6H). LCMS (ESI): m/z, 563.4 [M+H]⁺.

### Example 97. Preparation of Compound E20

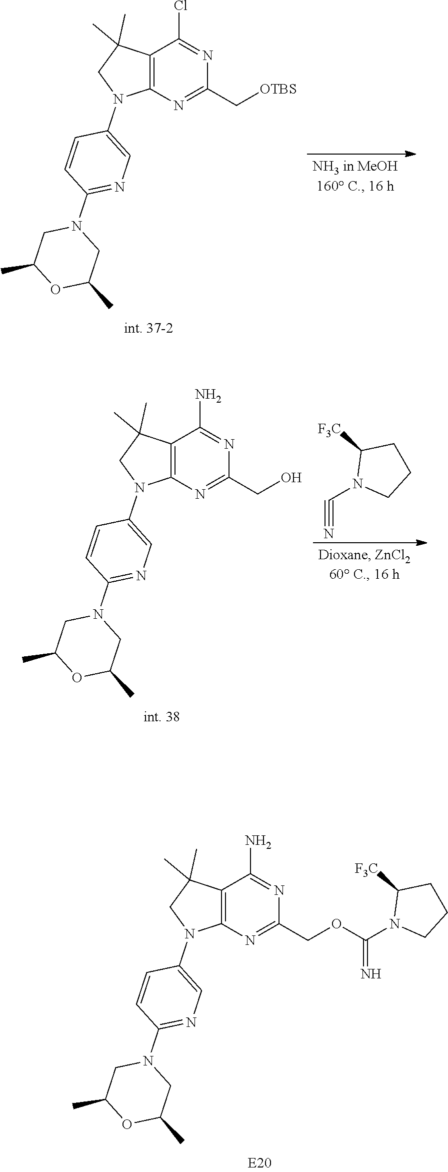 [SMILES: C[C@@H]1CN(c2ccc(N3CC(C)(C)c4c(Cl)nc(CO[Si](C)(C)C(C)(C)C)nc43)cn2)C[C@H](C)O1 ; C[C@@H]1CN(c2ccc(N3CC(C)(C)c4c(N)nc(CO)nc43)cn2)C[C@H](C)O1 ; N#CN1CCC[C@@H]1C(F)(F)F ; C[C@@H]1CN(c2ccc(N3CC(C)(C)c4c(N)nc(COC(=N)N5CCC[C@@H]5C(F)(F)F)nc43)cn2)C[C@H](C)O1]

1. Synthesis of compound int. 38. To a solution of (2R,6S)-4-(5-(2-(((tert-butyldimethylsilyl)oxy)methyl)-4-chloro-5,5-dimethyl-5,6-dihydro-7H-pyrrolo[2,3-d]pyrimidin-7-yl)pyridin-2-yl)-2,6-dimethylmorpholine (240 mg, 0.46 mmol) in NH₃ (7 M in MeOH, 40 mL). The reaction was stirred at 160° C. for 72 h. The mixture was diluted with water and extracted with EtOAc, the organic layer was washed with brine, dried over Na₂SO₄, filtered and concentrated. The residue was purified by column chromatography to give title compound (9 mg). LCMS (ESI): m/z, 385.2 [M+H]⁺.

2. Synthesis of compound E20. Compound E20 was synthesized as compound E16. ¹H NMR (400 MHz, CDCl₃) δ 8.37 (d, J=2.8 Hz, 1H), 7.89 (dd, J=2.8, 9.2 Hz, 1H), 6.71 (d, J=9.2 Hz, 1H), 5.22 (d, J=13.2 Hz, 1H), 5.17-5.04 (m, 3H), 4.74-4.67 (m, 1H), 3.98 (dd, J=2.4, 12.8 Hz, 2H), 3.75 (dt, J=2.4, 6.4 Hz, 2H), 3.69 (s, 2H), 2.53-2.47 (m, 2H), 2.22-2.09 (m, 4H), 1.43 (s, 6H), 1.27 (d, J=6.4 Hz, 6H). LCMS (ESI): m/z, 549.3 [M+H]⁺.

### Example 98. Preparation of Compound E21

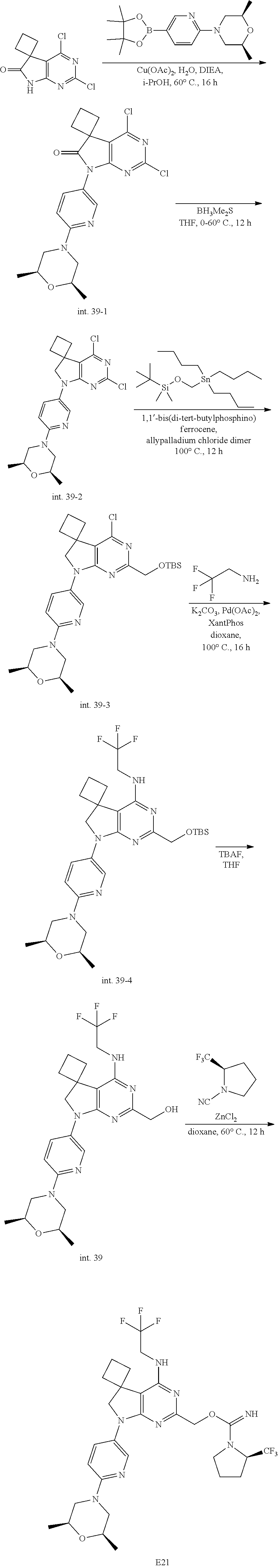

1. Synthesis of compound int. 39-1. A mixture of 2′,4′-dichlorospiro[cyclobutane-1,5′-pyrrolo[2,3-d]pyrimidin]-6′(7′H)-one (600 mg, 2.46 mmol), (2S,6R)-2,6-dimethyl-4-(5-(4,4,5,5-tetramethyl-1,3,2-dioxaborolan-2-yl)pyridin-2-yl)morpholine (1.56 g, 4.92 mmol), Cu(OAc)₂·H₂O (589 mg, 2.95 mmol), 4 Å MS (2.17 g) and DIPEA (953 mg, 1.22 mL, 7.37 mmol) in i-PrOH (20 mL) was stirred at 60° C. for 16 h under O₂ atmosphere. The mixture was filtered and concentrated. The residue was purified by column chromatography to give title compound (808 mg). LCMS (ESI): m/z, 434.2 [M+H]⁺.

2. Synthesis of compound int. 39-2. To a solution of 2′,4′-dichloro-7′-(6-((2S,6R)-2,6-dimethylmorpholino)pyridin-3-yl)spiro[cyclobutane-1,5′-pyrrolo[2,3-d]pyrimidin]-6′(7′H)-one (558 mg, 1.28 mmol) in THF (10 mL) was dropwise added BH₃.Me₂S (0.39 mL, 10 M in DMS) at 0° C., the mixture was stirred at 60° C. for 12 h. The mixture was quenched with MeOH (10 mL) at 0° C., then concentrated. The residue was purified by column chromatography to give title compound (302 mg). LCMS (ESI): m/z, 420.2 [M+H]⁺.

3. Synthesis of compound int. 39-3. A mixture of Bu₃SnCH₂OTBS (313 mg, 0.72 mmol), (2S,6R)-4-(5-(2′,4′-dichlorospiro[cyclobutane-1,5′-pyrrolo[2,3-d]pyrimidin]-7′(6′H)-yl)pyridin-2-yl)-2,6-dimethylmorpholine (302 mg, 0.72 mmol), allylpalladium chloride dimer (11 mg, 0.03 mmol) and 1,1′-bis(di-tert-butylphosphino)ferrocene (27 mg, 0.06 mmol) in 2-Methyl-2-butanol (6 mL) was stirred at 100° C. for 12 h. The mixture was filtered and concentrated. The residue was purified by column chromatography to give title compound (142 mg). LCMS (ESI): m/z, 530.3 [M+H]⁺.

4. Synthesis of compound int. 39-4. A mixture of (2S,6R)-4-(5-(2′-(((tert-butyldimethylsilyl)oxy)methyl)-4′-chlorospiro[cyclobutane-1,5′-pyrrolo[2,3-d]pyrimidin]-7′(6′H)-yl)pyridin-2-yl)-2,6-dimethylmorpholine (120 mg, 0.23 mmol), 2,2,2-trifluoroethan-1-amine (45 mg, 0.45 mmol), K₂CO₃ (63 mg, 0.45 mmol), Pd(OAc)₂ (4 mg, 0.02 mmol) and XantPhos (26 mg, 0.45 mmol) in 1,4-dioxane (2.5 mL) was stirred at 100° C. for 16 h under N₂ atmosphere. The mixture was filtered and concentrated. The mixture was diluted with DCM and washed with water, brine, the organic layer was dried over Na₂SO₄, filtered and concentrated. The residue was purified by column chromatography to give title compound (180 mg crude).

5. Synthesis of compound int. 39. To a solution of 2′-(((tert-butyldimethylsilyl)oxy)methyl)-7′-(6-((2S,6R)-2,6-dimethylmorpholino)pyridin-3-yl)-N-(2,2,2-trifluoroethyl)-6′,7′-dihydrospiro[cyclobutane-1,5′-pyrrolo[2,3-d]pyrimidin]-4′-amine (170 mg, 0.29 mmol) in THF (5 mL) was added TBAF (0.86 mmol, 1 M in THF), the mixture was stirred at 25° C. for 0.5 hour. The mixture was diluted with EtOAc and washed with water, brine, the organic layer was dried over Na₂SO₄, filtered and concentrated. The residue was purified by column chromatography to give title compound (61 mg). LCMS (ESI): m/z, 479.3 [M+H]⁺.

6. Synthesis of compound E21. Compound E21 was synthesized as compound E16. LCMS (ESI): m/z, 643.4 [M+H]⁺.

### Example 99. Preparation of Compound E22

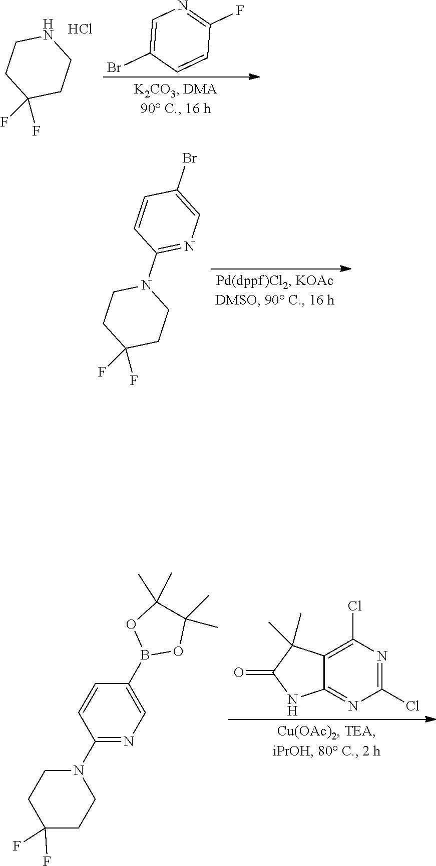 [SMILES: FC1(F)CCNCC1 ; Cl ; Fc1ccc(Br)cn1 ; FC1(F)CCN(c2ccc(Br)cn2)CC1 ; CC1(C)OB(c2ccc(N3CCC(F)(F)CC3)nc2)OC1(C)C ; CC1(C)C(=O)Nc2nc(Cl)nc(Cl)c21]

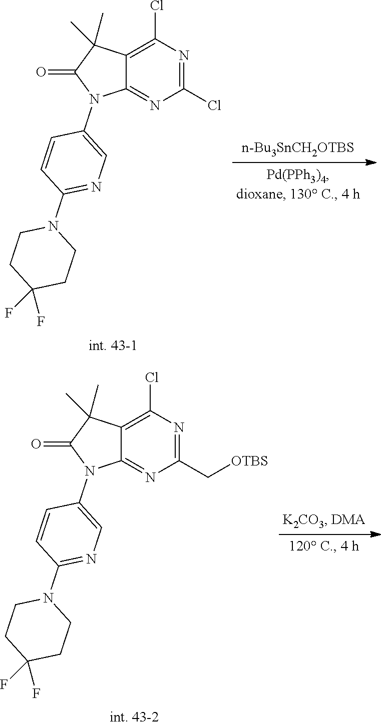

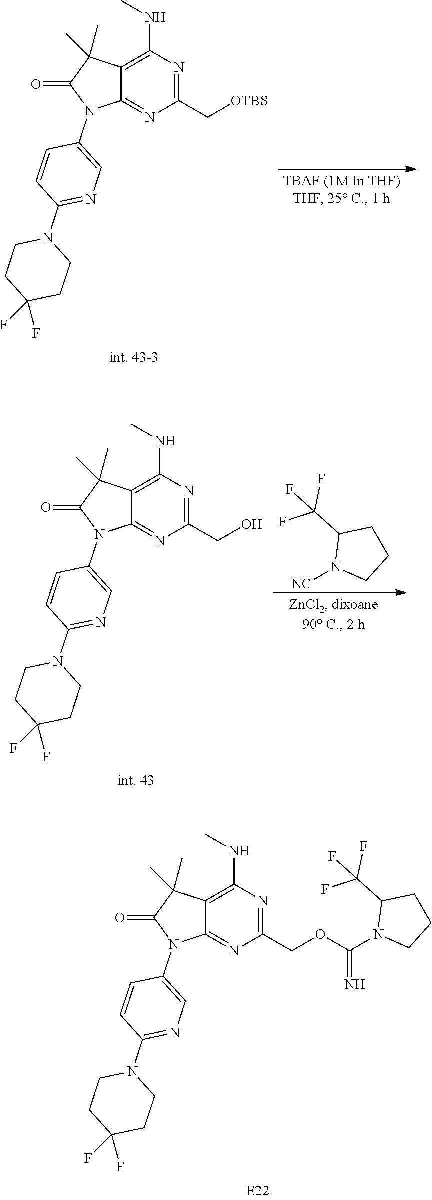

1. Synthesis of compound 5-bromo-2-(4,4-difluoropiperidin-1-yl)pyridine. To a solution of 4,4-difluoropiperidine hydrochloride (3.0 g, 19.0 mmol), 5-bromo-2-fluoro-pyridine (3.35 g, 19.0 mmol) in DMA (50 mL) was added K₂CO₃ (13.2 g, 95.2 mmol). The mixture was stirred at 90° C. for 16 h. The mixture was filtered, the filtrate was diluted with EA (200 mL), washed with sat. aq. NaCl, dried, and concentrated. The residue was purified by column chromatography to afford 5-bromo-2-(4,4-difluoropiperidin-1-yl)pyridine (4.6 g). ¹H NMR (400 MHz, CDCl₃) δ 8.19 (d, J=2.4 Hz, 1H), 7.55 (dd, J=2.8, 9.2 Hz, 1H), 6.60 (dd, J=0.8, 9.2 Hz, 1H), 3.71-3.68 (m, 4H), 2.06-1.96 (m, 4H). LCMS (ESI): m/z, 276.9, 279.0 [M+H]⁺.

2. Synthesis of compound 2-(4,4-difluoropiperidin-1-yl)-5-(4,4,5,5-tetramethyl-1,3,2-dioxaborolan-2-yl)pyridine. A mixture of 5-bromo-2-(4,4-difluoro-1-piperidyl)pyridine (4.50 g, 16.2 mmol), 4,4,5,5-tetramethyl-2-(4,4,5,5-tetramethyl-1,3,2-dioxaborolan-2-yl)-1,3,2-dioxaborolane (8.25 g, 32.5 mmol), Pd(dppf)Cl₂ (1.19 g, 1.62 mmol), and KOAc (3.19 g, 32.5 mmol) in DMSO (50 mL) was stirred at 90° C. for 12 h under N₂ atmosphere. The mixture was diluted with EA (200 mL) and filtered. The filtrate was washed with sat. aq. NaCl, dried over Na₂SO₄, filtered and concentrated. The residue was purified by column chromatography to afford 2-(4,4-difluoropiperidin-1-yl)-5-(4,4,5,5-tetramethyl-1,3,2-dioxaborolan-2-yl)pyridine (4.6 g, 87.3% yield) as off-white solid. ¹H NMR (400 MHz, CDCl₃) δ 8.35 (d, J=2.0 Hz, 1H), 7.72 (dd, J=2.0, 9.2 Hz, 1H), 6.92 (dd, J=0.8, 8.8 Hz, 1H), 3.76-3.76 (m, 4H), 2.01-1.91 (m, 4H), 1.27 (s, 12H). LCMS (ESI): m/z, 243.1 [M+H]⁺.

3. Synthesis of compound int. 43-1. A mixture of 2,4-dichloro-5,5-dimethyl-7H-pyrrolo[2,3-d]pyrimidin-6-one (1.0 g, 4.31 mmol), 2-(4,4-difluoro-1-piperidyl)-5-(4,4,5,5-tetramethyl-1,3,2-dioxaborolan-2-yl)pyridine (2.10 g, 6.46 mmol), 4 Å MS (3.80 g), Cu(OAc)·H₂O (860 mg, 4.31 mmol), and TEA (1.20 mL) in iPrOH (30 mL) was stirred at 80° C. for 2 h under 02 atmosphere. The mixture was concentrated. The residue was purified by column chromatography to afford 2,4-dichloro-7-(6-(4,4-difluoropiperidin-1-yl)pyridin-3-yl)-5,5-dimethyl-5,7-dihydro-6H-pyrrolo[2,3-d]pyrimidin-6-one (700 mg). LCMS (ESI): m/z, 428.1 [M+H]⁺.

4. Synthesis of compound int. 43-2. A mixture of 2,4-dichloro-7-(6-(4,4-difluoropiperidin-1-yl)pyridin-3-yl)-5,5-dimethyl-5,7-dihydro-6H-pyrrolo[2,3-d]pyrimidin-6-one (700 mg, 1.63 mmol), Pd(Ph₃)₄ (189 mg, 0.163 mmol), and tert-butyl-dimethyl-(tributylstannylmethoxy)silane (854 mg, 1.96 mmol) in dioxane (10 mL) was stirred at 130° C. for 4 h under N₂ atmosphere. The mixture was concentrated. The residue was purified by column chromatography to afford 2-(((tert-butyldimethylsilyl)oxy)methyl)-4-chloro-7-(6-(4,4-difluoropiperidin-1-yl)pyridin-3-yl)-5,5-dimethyl-5,7-dihydro-6H-pyrrolo[2,3-d]pyrimidin-6-one (700 mg). LCMS (ESI): m/z, 538.3 [M+H]⁺.

5. Synthesis of compound int. 43-3. A mixture of 2-(((tert-butyldimethylsilyl)oxy)methyl)-4-chloro-7-(6-(4,4-difluoropiperidin-1-yl)pyridin-3-yl)-5,5-dimethyl-5,7-dihydro-6H-pyrrolo[2,3-d]pyrimidin-6-one (700 mg, 1.30 mmol), K₂CO₃ (539 mg, 3.90 mmol), and methanamine hydrochloride (264 mg, 3.90 mmol) in DMA (10 mL) was stirred at 120° C. for 4 h under N₂ atmosphere. The mixture was diluted with water (10 mL), and extracted with EtOAc, washed with brine. The organic phase was dried over Na₂SO₄, filtered and concentrated to afford 2-(((tert-butyldimethylsilyl)oxy)methyl)-7-(6-(4,4-difluoropiperidin-1-yl)pyridin-3-yl)-5,5-dimethyl-4-(methylamino)-5,7-dihydro-6H-pyrrolo[2,3-d]pyrimidin-6-one (690 mg crude). LCMS (ESI): m/z, 533.3 [M+H]⁺.

6. Synthesis of compound int. 43. To a solution of 2-(((tert-butyldimethylsilyl)oxy)methyl)-7-(6-(4,4-difluoropiperidin-1-yl)pyridin-3-yl)-5,5-dimethyl-4-(methylamino)-5,7-dihydro-6H-pyrrolo[2,3-d]pyrimidin-6-one (650 mg, 0.610 mmol) in THF (14 mL) was added TBAF (1 M in THF, 1.83 mL). The mixture was stirred at 25° C. for 1 h. The mixture was concentrated. The residue was purified by column chromatography to afford 7-(6-(4,4-difluoropiperidin-1-yl)pyridin-3-yl)-2-(hydroxymethyl)-5,5-dimethyl-4-(methylamino)-5,7-dihydro-6H-pyrrolo[2,3-d]pyrimidin-6-one (160 mg). LCMS (ESI): m/z, 419.2 [M+H]⁺

7. Synthesis of compound E22. Compound E22 was synthesized as compound E15. ¹H NMR (400 MHz, DMSO-d₆) (8.13 (d, J=2.4 Hz, 1H), 7.58 (dd, J=9.2, 2.8 Hz, 1H), 7.03 (d, J=9.2 Hz, 1H), 6.73 (s, 1H), 5.86 (s, 1H), 5.03-4.90 (m, 2H), 4.58-4.52 (m, 1H), 3.74-3.72 (m, 4H), 2.90 (d, J=4.4 Hz, 3H), 2.07-1.83 (m, 10H), 1.42 (s, 6H). LCMS (ESI): m/z, 583.2 [M+H]⁺.

### Example 100. Preparation of Compound E23

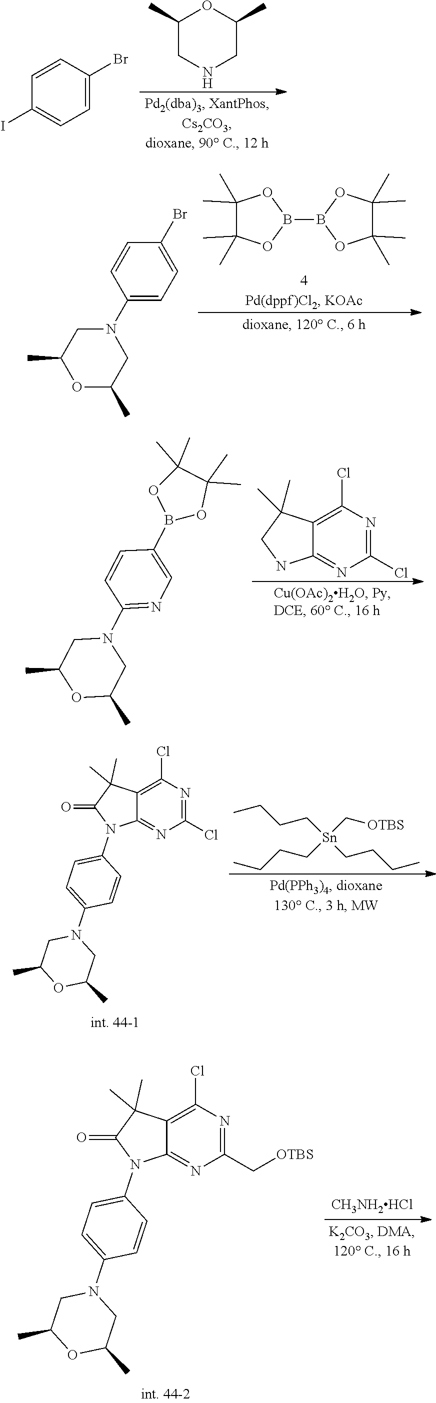

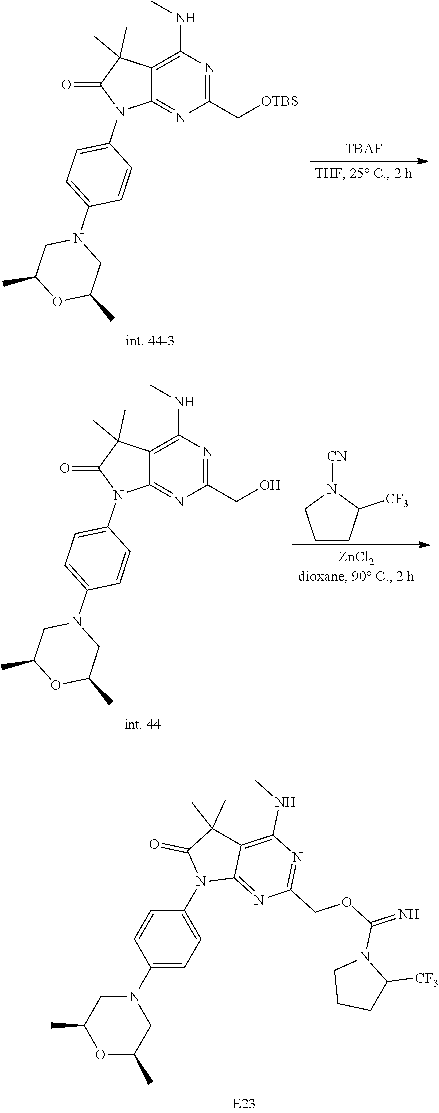

1. Synthesis of compound (2R,6S)-4-(4-bromophenyl)-2,6-dimethylmorpholine. A mixture of 1-bromo-4-iodo-benzene (1 g, 3.53 mmol), (2R,6S)-2,6-dimethylmorpholine (326 mg, 2.83 mmol), Pd₂(dba)₃ (324 mg, 0.35 mmol), XantPhos (409 mg, 0.71 mmol) and Cs₂CO₃ (2.88 g, 8.84 mmol) in dioxane (15 mL) was stirred at 90° C. for 12 h under N₂. The reaction mixture was diluted with H₂O and extracted with EtOAc. The combined organic phase was washed with brine, dried over anhydrous Na₂SO₄, filtered and concentrated. The residue was purified by column chromatography to give title compound (0.7 g). LCMS (ESI): m/z, 270.1 [M+H]⁺.

2. Synthesis of compound (2R,6S)-2,6-dimethyl-4-(4-(4,4,5,5-tetramethyl-1,3,2-dioxaborolan-2-yl)phenyl)morpholine. To a solution of (2S,6R)-4-(4-bromophenyl)-2,6-dimethyl-morpholine (0.7 g, 2.59 mmol), 4,4,4′,4′,5,5,5′,5′-octamethyl-2,2′-bi(1,3,2-dioxaborolane) (790 mg, 3.11 mmol) in DMF (12 mL) was added Pd(dppf)Cl₂ (190 mg, 0.26 mmol) and KOAc (636 mg, 6.48 mmol). The mixture was stirred at 100° C. for 12 h. The reaction was filtered and concentrated. The residue was purified by column chromatography to give (0.55 g). ¹H NMR (400 MHz, CDCl₃) δ 7.73-7.69 (m, 2H), 6.87 (d, J=8.0 Hz, 2H), 3.79 (s, 2H), 3.56-3.53 (m, 2H), 2.45 (t, J=11.2 Hz, 2H), 1.33 (s, 12H), 1.25 (s, 6H). LCMS (ESI): t_{R}=3.818, m/z, 318.2 [M+H]⁺.

3. Synthesis of compound int. 44-1. To a solution of 2,4-dichloro-5,5-dimethyl-6,7-dihydro-5H-pyrrolo[2,3-d]pyrimidine (0.50 g, 2.15 mmol) and (2R,6S)-2,6-dimethyl-4-(4-(4,4,5,5-tetramethyl-1,3,2-dioxaborolan-2-yl)phenyl)morpholine (1.37 g, 4.31 mmol) in iPrOH (5 mL) were added cupric acetate monohydrate (516 mg, 2.59 mmol) and 4 Å MS (1.5 g). The mixture was stirred at 60° C. for 16 h under O₂ (15 psi) atmosphere. The mixture was filtered and the filtrate was concentrated. The residue was purified by column chromatography to give 2,4-dichloro-7-(4-((2R,6S)-2,6-dimethylmorpholino)phenyl)-5,5-dimethyl-5,7-dihydro-6H-pyrrolo[2,3-d]pyrimidin-6-one (471 mg). LCMS (ESI): m/z, 421.1, 423.1 [M+H]⁺.

4. Synthesis of compound int. 44-2. To a solution of 2,4-dichloro-7-(4-((2R,6S)-2,6-dimethylmorpholino)phenyl)-5,5-dimethyl-5,7-dihydro-6H-pyrrolo[2,3-d]pyrimidin-6-one (170 mg, 0.40 mmol) and tert-butyldimethyl((tributylstannyl)methoxy)silane (193 mg, 0.44 mmol) in dioxane (5 mL) was added Pd(PPh₃)₄ (140 mg, 0.12 mmol) and stirred at 130° C. for 3 h under microwave. The reaction was filtered and concentrated under reduce pressure to give a residue which was purified by column chromatography to give 2-(((tert-butyldimethylsilyl)oxy)methyl)-4-chloro-7-(4-((2R,6S)-2,6-dimethylmorpholino)phenyl)-5,5-dimethyl-5,7-dihydro-6H-pyrrolo[2,3-d]pyrimidin-6-one (80 mg). LCMS (ESI): m/z, 531.3 [M+H]⁺.

5. Synthesis of compound int. 44-3. To a solution of 2-(((tert-butyldimethylsilyl)oxy)methyl)-4-chloro-7-(4-((2R,6S)-2,6-dimethylmorpholino)phenyl)-5,5-dimethyl-5,7-dihydro-6H-pyrrolo[2,3-d]pyrimidin-6-one (80 mg, 0.15 mmol), methylamine hydrochloride (25 mg, 0.38 mmol) in DMA (1 mL) was added K₂CO₃ (104 mg, 0.75 mmol).The mixture was stirred at 120° C. for 16 h. The reaction was filtered and concentrated under reduce pressure to give 2-(((tert-butyldimethylsilyl)oxy)methyl)-7-(4-((2R,6S)-2,6-dimethylmorpholino)phenyl)-5,5-dimethyl-4-(methylamino)-5,7-dihydro-6H-pyrrolo[2,3-d]pyrimidin-6-one (80 mg crude). LCMS (ESI): m/z, 526.4 [M+H]⁺.

6. Synthesis of compound int. 44. To a solution of 2-(((tert-butyldimethylsilyl)oxy)methyl)-7-(4-((2R,6S)-2,6-dimethylmorpholino)phenyl)-5,5-dimethyl-4-(methylamino)-5,7-dihydro-6H-pyrrolo[2,3-d]pyrimidin-6-one (80 mg, 0.15 mmol) in THF (5 mL) was added TBAF (1 M in THF, 0.61 mL). The mixture was stirred at 25° C. for 2 h, water (50 mL) was added and extracted with EtOAc. The combined organic phase was washed with brine, dried over anhydrous Na₂SO₄, filtered and concentrated. The residue was purified by column chromatography to give 7-(4-((2R,6S)-2,6-dimethylmorpholino)phenyl)-2-(hydroxymethyl)-5,5-dimethyl-4-(methylamino)-5,7-dihydro-6H-pyrrolo[2,3-d]pyrimidin-6-one (23 mg). LCMS (ESI): m/z, 412.2 [M+H]⁺.

7. Synthesis of compound E23. Compound E23 was synthesized as compound E15. LCMS (ESI): m/z, 576.3 [M+H]⁺. ¹H NMR (400 MHz, CDCl₃) δ 7.37-7.31 (m, 2H), 6.97-6.91 (m, 2H), 5.21 (d, J=14.8 Hz, 1H), 5.03 (d, J=14.8 Hz, 1H), 4.54-4.44 (m, 1H), 4.38 (s, 1H), 3.86-3.76 (m, 2H), 3.52-3.36 (m, 4H), 3.08 (d, J=4.8 Hz, 3H), 2.44 (m, 2H), 2.07-2.02 (m, 2H), 1.89 (m, 2H), 1.48 (s, 6H), 1.27 (d, J=6.4 Hz, 6H).

### Example 101. Preparation of Compound E24

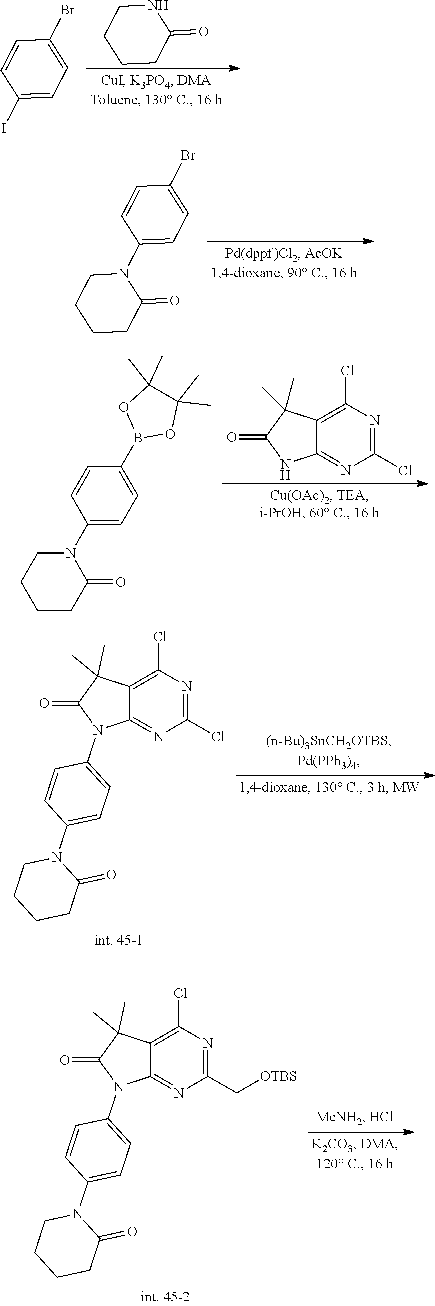

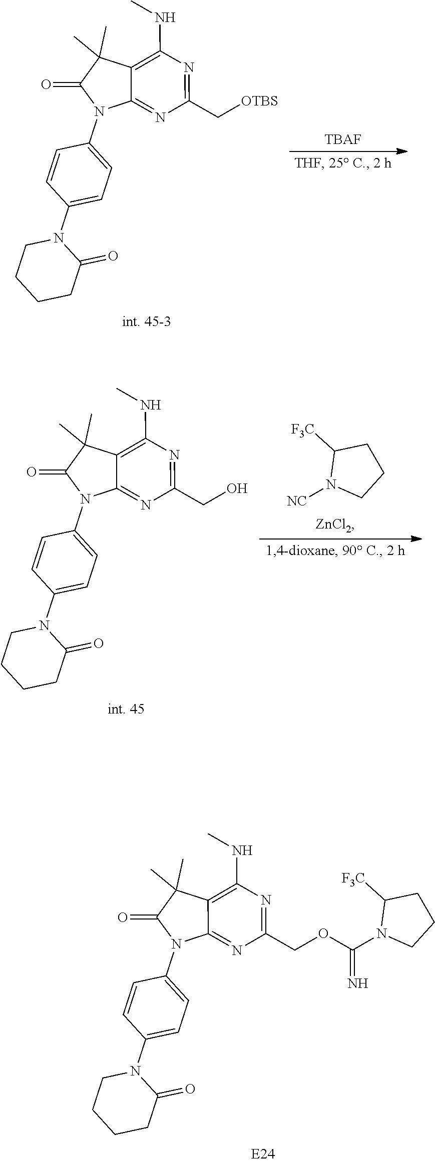

1. Synthesis of compound 1-(4-bromophenyl) piperidin-2-one. To a solution of 1-bromo-4-iodo-benzene (5.0 g, 17.7 mmol) and piperidin-2-one (1.75 g, 17.7 mmol) were added tripotassium phosphate (7.50 g, 35.3 mmol), cuprous iodide (169 mg, 0.88 mmol) and N, N′-dimethylethane-1,2-diamine (78 mg, 0.88 mmol). The reaction was stirred at 130° C. for 16 h under N₂ atmosphere. The reaction was filtered and concentrated. The residue was purified by column chromatography to give title compound (2.08 g). ¹H NMR (400 MHz, DMSO-d₆) δ 7.58-7.53 (m, 2H), 7.27-7.22 (m, 2H), 3.61-3.57 (m, 2H), 2.38 (t, J=6.4 Hz, 2H), 1.84 (m, 4H). LCMS (ESI): m/z, 253.9, 256.0 [M+H]⁺.

2. Synthesis of compound E24. Other steps of compound E24 were synthesized as compound E22. ¹H NMR (400 MHz, DMSO-d₆) δ 7.46-7.33 (m, 4H), 6.74 (d, J=4.8 Hz, 1H), 5.79 (s, 1H), 5.06-4.86 (m, 2H), 4.64-4.53 (m, 1H), 3.64 (t, J=5.6 Hz, 2H), 2.90 (d, J=4.4 Hz, 3H), 2.42 (t, J=6.4 Hz, 2H), 1.87 (q, J=6.8 Hz, 8H), 1.44 (s, 6H). LCMS (ESI): m/z, 560.3 [M+H]⁺.

### Example 102. Preparation of Compound E25

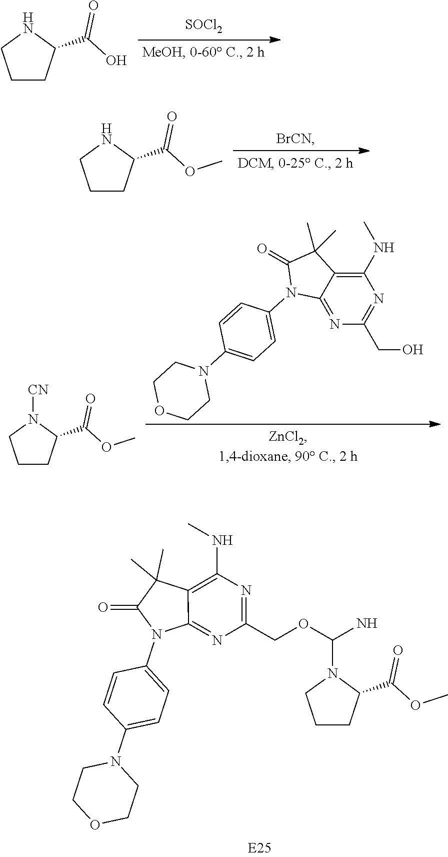

1. Synthesis of compound methyl L-prolinate hydrochloride. To a mixture of L-proline (3.70 mL) in MeOH (50 mL) was added dropwise SOCl₂ (10.3 g, 86.8 mmol) at 0° C. and the mixture was stirred 0.5 h. Then the mixture was heated to 65° C. and stirred for 3 h. The mixture is directly concentrated to give title compound (7.0 g crude). LCMS (ESI): m/z, 129.9 [M+H]⁺.

2. Synthesis of compound methyl cyano-L-prolinate. To a mixture of methyl L-prolinate hydrochloride (1.50 g, 11.6 mmol) in DCM (10 mL) were added a solution of NaHCO₃ (4.88 g, 58.1 mmol) in H₂O (10 mL) and BrCN (1.48 g, 13.9 mmol) with stirring at 0° C., then the mixture was stirred at 25° C. for 2 h. The mixture was filtered. The residue was purified by column chromatography to give methyl cyano-L-prolinate (390 mg). ¹H NMR (400 MHz, CDCl₃) (δ 4.24 (dd, J=4.0, 8.4, Hz, 1H), 3.80 (s, 3H), 3.62 (dt, J=6.4, 8.8 Hz, 1H), 3.50 (dt, J=7.2, 9.2 Hz, 1H), 2.31-2.18 (m, 1H), 2.16-2.05 (m, 1H), 2.03-1.88 (m, 2H). LCMS (ESI): m/z, 154.9 [M+H]⁺.

3. Synthesis of compound E25. Compound E25 was synthesized as compound E15. ¹H NMR (400 MHz, CDCl₃) δ 7.29 (dd, J=3.6, 8.8 Hz, 2H), 7.01 (dd, J=2.0, 8.8 Hz, 2H), 5.32-5.09 (m, 2H), 4.89 (d, J=4.8 Hz, 1H), 4.47 (dd, J=8.4, 3.6 Hz, 1H), 3.92-3.80 (m, 5H), 3.75 (d, J=14.4 Hz, 4H), 3.28-3.16 (m, 4H), 3.08 (d, J=4.4 Hz, 3H), 2.16 (dd, J=8.4, 12.8 Hz, 2H), 1.51 (s, 6H). LCMS (ESI): t_{R}=1.968, m/z, 538.3 [M+H]⁺.

### Example 103. Preparation of Compound E26

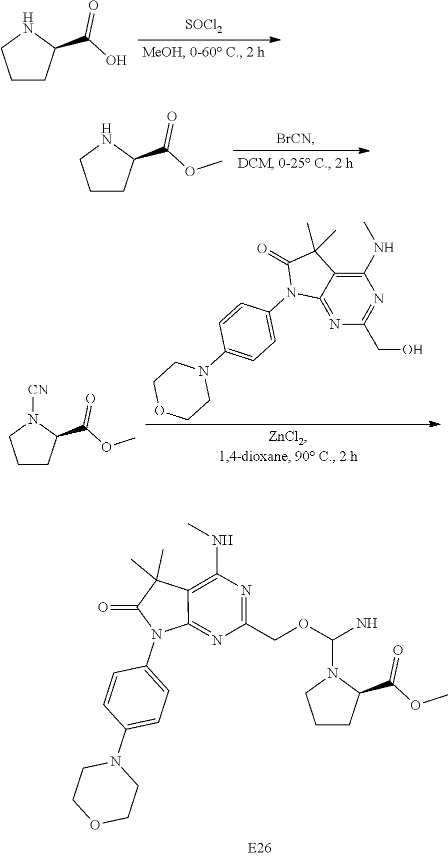

Compound E26 was synthesized as compound E25. ¹H NMR (400 MHz, CDCl₃) δ 7.29 (dd, J=3.6, 8.8 Hz, 2H), 7.02 (d, J=8.8 Hz, 2H), 5.36-5.09 (m, 2H), 4.99 (s, 1H), 4.48 (dd, J=3.6, 8.4 Hz, 1H), 4.04-3.81 (m, 5H), 3.75 (d, J=11.2 Hz, 4H), 3.35-3.17 (m, 4H), 3.06 (s, 3H), 2.35-2.09 (m, 2H), 1.51 (s, 6H). LCMS (ESI): m/z, 538.3 [M+H]⁺.

### Example 104. Preparation of Compound E27

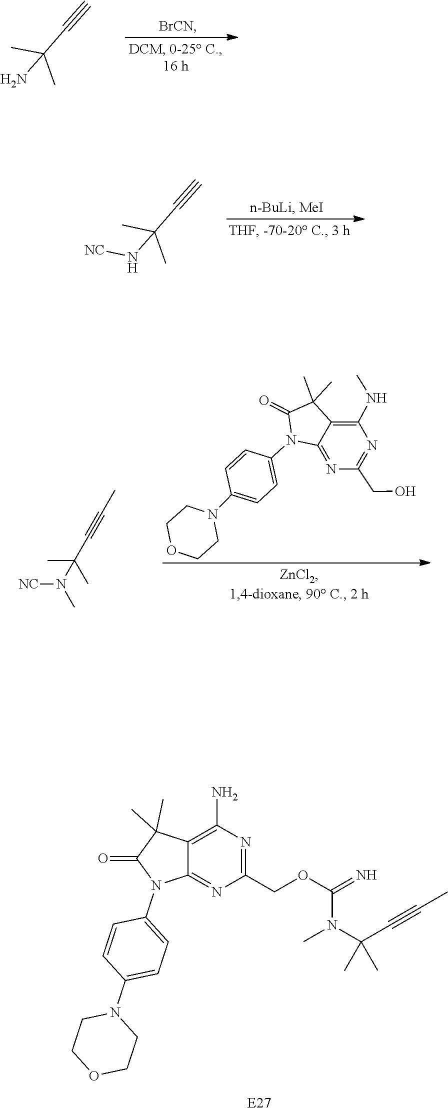

1. Synthesis of compound N-(2-methylbut-3-yn-2-yl)cyanamide. To a solution of 2-methylbut-3-yn-2-amine (1.0 g, 12.0 mmol) in DCM (10 mL) was added sat. aq. NaHCO₃ (21.6 g, 257 mmol) at 0° C., then a solution of BrCN (1.91 g, 18.0 mmol) in DCM (18 mL) was added dropwise to the mixture, the mixture was stirred at 25° C. for 12 h. The mixture was quenched with sat. aq. NaHCO₃ (30 mL) and extracted with DCM, the organic layer was washed with brine, dried over Na₂SO₄, filtered and concentrated to give compound N-(2-methylbut-3-yn-2-yl)cyanamide (726 mg). ¹H NMR (400 MHz, CDCl₃) δ 3.91 (s, 1H), 2.49 (s, 2H), 1.59 (s, 3H). LCMS (ESI): m/z, 109.1 [M+H]⁺.

2. Synthesis of compound N-methyl-N-(2-methylpent-3-yn-2-yl)cyanamide. N-(2-methylbut-3-yn-2-yl)cyanamide (100 mg, 0.925 mmol) in THF (2 mL) was cooled to −70° C. under N₂ atmosphere with stirring and a solution of n-BuLi (1.27 mL, 1.60 M) was added at −70° C. during 15 min. The mixture was allowed to warm to −5° C. and a solution of CH₃I (394 mg, 2.77 mmol) in THF (1 mL) was added dropwise. The mixture was allowed to warm to 20° C. for 2 h. The mixture was adjusted to pH=6 by sat. aq. NH₄Cl, then extracted with EtOAc, the organic layer was washed with brine, dried over Na₂SO₄, filtered and concentrated to give compound N-methyl-N-(2-methylpent-3-yn-2-yl)cyanamide (245 mg). ¹H NMR (400 MHz, CDCl₃) δ 2.91 (s, 3H), 1.84 (s, 3H), 1.53 (s, 6H). LCMS (ESI): m/z, 137.2 [M+H]⁺.

3. Synthesis of compound E27. Compound E27 was synthesized as compound E15. ¹H NMR (400 MHz, CDCl3) δ 7.22 (d, J=8.8 Hz, 2H), 7.03 (d, J=8.8 Hz, 2H), 6.79 (s, 2H), 4.94 (s, 2H), 3.78 (t, J=4.8 Hz, 4H), 3.17 (t, J=4.8 Hz, 4H), 2.81 (s, 3H), 1.80 (s, 3H), 1.45 (d, J=6.0 Hz, 12H). LCMS (ESI): m/z, 506.4 [M+H]⁺.

### Example 105. Preparation of Compound E28

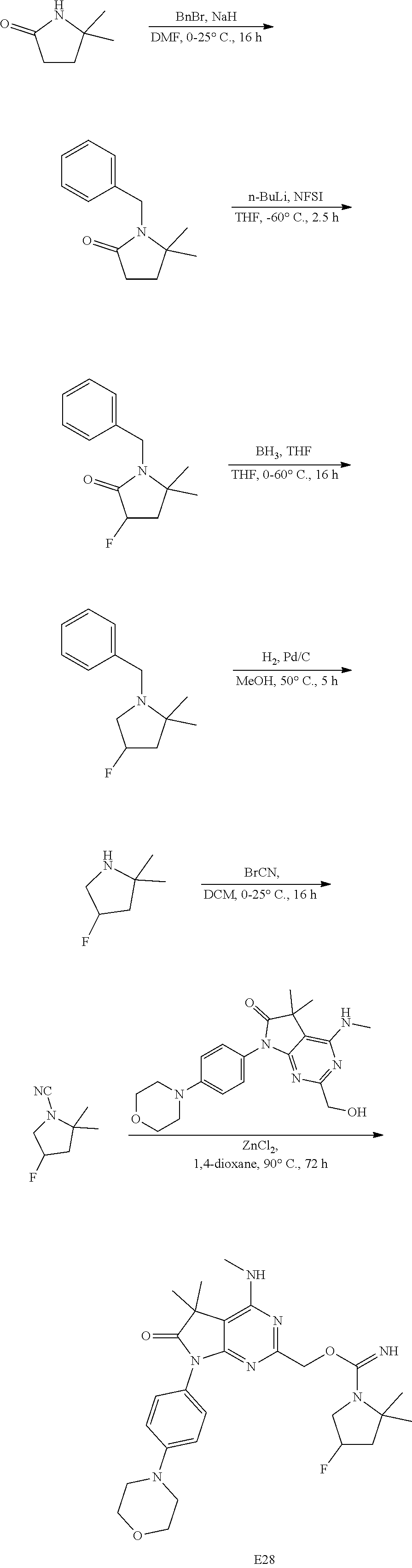

1. Synthesis of compound 1-benzyl-5,5-dimethyl-pyrrolidin-2-one. To a solution of 5,5-dimethylpyrrolidin-2-one (1.0 g, 8.84 mmol) in DMF (10 mL) was added NaH (707 mg, 17.7 mmol, 60% purity) at 0° C. and stirred for 30 min. Bromomethylbenzene (1.81 g, 10.6 mmol) was added dropwise at 0° C. and stirred at 25° C. for 16 h. The mixture was poured into sat. aq. NH₄Cl (50 mL) slowly and extracted with EtOAc. The combined organic phases were concentrated. The residue was purified by column chromatography to give 1-benzyl-5,5-dimethyl-pyrrolidin-2-one (1.8 g). LCMS (ESI): m/z, 204.1 [M+H]⁺.

2. Synthesis of compound 1-benzyl-3-fluoro-5,5-dimethyl-pyrrolidin-2-one. To a solution of 1-benzyl-5,5-dimethyl-pyrrolidin-2-one (1.5 g, 7.38 mmol) in THF (50 mL) was added n-BuLi (13.8 mL, 1.6 M) dropwise at −60° C. and stirred at −60° C. for 30 min. A solution of NFSI (5.82 g, 18.45 mmol) in THF (1 mL) was added dropwise at −60° C. The mixture was stirred at −60° C. for 2 h. The reaction was quenched by sat.aq. NH₄Cl (50 mL), and water (20 mL) was added and extracted with EtOAc. The combined organic phases were washed with brine, dried and concentrated. The residue was purified by Prep-HPLC to yield title compound (800 mg). ¹H NMR (400 MHz, CDCl₃) δ 7.32-7.21 (m, 5H), 5.23-4.39 (ddd, J=5.6, 7.6, 52.8 Hz, 1H), 4.57-4.37 (m, 2H), 2.38-1.97 (m, 2H), 1.20 (s, 3H), 1.11 (s, 3H).

3. Synthesis of compound 1-benzyl-4-fluoro-2,2-dimethyl-pyrrolidine. To a solution of 1-benzyl-3-fluoro-5,5-dimethyl-pyrrolidin-2-one (500 mg, 2.26 mmol) in THF (3 mL) was added BH₃ (11.3 mL, 1 M in THF) dropwise at 0° C. and stirred at 60° C. for 16 h. The reaction was quenched by MeOH (5 mL), then was concentrated, and the residue was treated with EtOAc (5 mL). The mixture was filtered, and the filtrate was concentrated. The residue was purified by Prep-TLC to give 1-benzyl-4-fluoro-2,2-dimethyl-pyrrolidine (300 mg). ¹H NMR (400 MHz, CDCl₃) δ 7.38-7.24 (m, 5H), 5.08 (dtt, J=2.4, 6.4, 56.6 Hz, 1H), 3.65-3.49 (m, 2H), 3.04-2.95 (m, 1H), 2.87-2.76 (m, 1H), 2.13-1.88 (m, 2H), 1.22 (s, 3H), 1.11 (s, 3H). LCMS (ESI): m/z, 208.2 [M+H]⁺.

4. Synthesis of compound 4-fluoro-2,2-dimethyl-pyrrolidine. To a solution of 1-benzyl-4-fluoro-2,2-dimethyl-pyrrolidine (300 mg, 1.45 mmol) in MeOH (5 mL) was added Pd/C (20 mg). The suspension was degassed under vacuum and purged with H₂ for 3 times. The mixture was stirred under H₂ (15 psi) at 50° C. for 5 h. The mixture was filtered and the filtrate was treated with HCl aq. (2M, 1 mL). The mixture was concentrated and the residue was treated with water (10 mL), ACN (5 mL), then lyophilization to yield 4-fluoro-2,2-dimethyl-pyrrolidine (222 mg). ¹H NMR (400 MHz, CD₃OD) δ 5.51-5.35 (m, 1H), 3.75-3.59 (m, 1H), 2.33-2.29 (m, 1H), 2.25-2.20 (m, 1H), 1.57 (s, 3H), 1.50 (s, 3H).

5. Synthesis of compound 4-fluoro-2,2-dimethyl-pyrrolidine-1-carbonitrile. To a solution of 4-fluoro-2,2-dimethyl-pyrrolidine (250 mg, 2.13 mmol) in THF (5 mL) were added sat. aq. NaHCO₃ (12.5 mL) and BrCN (294 mg, 2.77 mmol) at 0° C. and the mixture was stirred at 25° C. for 16 h. The mixture was concentrated. The residue was purified by column chromatography to give 4-fluoro-2,2-dimethyl-pyrrolidine-1-carbonitrile (120 mg). ¹H NMR (400 MHz, CDCl₃) δ 5.27-5.06 (m, 1H), 3.78 (d, J=2.8 Hz, 1H), 3.73-3.68 (m, 1H), 2.35-2.15 (m, 1H), 2.03-1.87 (m, 1H), 1.45 (s, 3H), 1.41 (s, 3H).

6. Synthesis of compound E28. Compound E28 was synthesized as compound E15.

### Example 106. Preparation of Compound E29

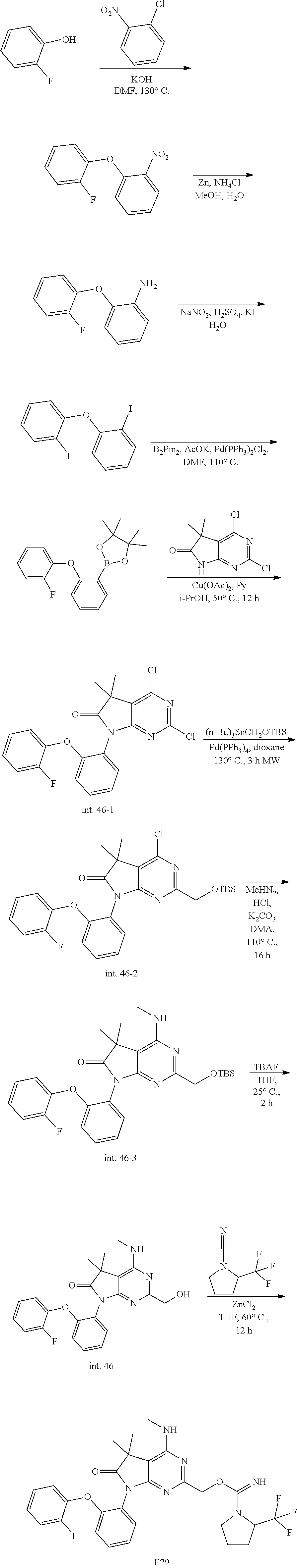

1. Synthesis of compound 2-fluorophenyl 2-nitrophenyl ether. To a mixture of 2-fluorophenol (10.0 g, 89.2 mmol) in DMF (100 mL) was added KOH (15.0 g, 268 mmol), the mixture was heated at 110° C. for 2 h. Then 2-nitrochlorobenzene (10.43 mL) was added and the reaction was stirred at 130° C. for 24 h. The mixture was filtered and the filtrate was treated with water and extracted with EtOAc. The combined organic phases were washed with brine, dried and concentrated. The residue was purified by column chromatography to give 2-fluorophenyl 2-nitrophenyl ether (20.0 g) LCMS (ESI): m/z, 233.9 [M+H]⁺.

2. Synthesis of compound 2-aminophenyl 2-fluorophenyl ether. To a mixture of 2-fluorophenyl 2-nitrophenyl ether (20.0 g, 85.8 mmol) and Zn (22.4 g, 343 mmol) in MeOH (100 mL) was added dropwise NH₄Cl (36.7 g, 686 mmol) in H₂O (100 mL) at 0° C., and the mixture was stirred at 25° C. for 6 h. The mixture was filtered, and the filtrate was concentrated. The residue was purified by column chromatography to give 2-aminophenyl 2-fluorophenyl ether (5.50 g). LCMS (ESI): m/z, 204.1 [M+H]⁺.

3. Synthesis of compound 2-fluorophenyl 2-iodophenyl ether. To a solution of H₂SO₄ (5 mL) in H₂O (30 mL) and MeCN (20 mL) was add 2-aminophenyl 2-fluorophenyl ether (4.90 g, 24.1 mmol), then a solution of NaNO₂ (1.83 g, 26.5 mmol) in H₂O (5 mL) was add dropwise at 0° C. The mixture was stirred at 0° C. for 0.5 h before KI (5.20 g, 31.35 mmol, 1.3 eq) in H₂O (5 mL) was added dropwise. Then the mixture was heated at 100° C. for 1 h. To the mixture was diluted with H₂O (100 mL) and extracted with EA (50 mL×3). The combined organic layers were dried over Na₂SO₄, filtered and concentrated. The residue was purified by column chromatography to give 2-fluorophenyl 2-iodophenyl ether (5.30 g). ¹H NMR (400 MHz, CDCl₃) δ 7.86 (dd, J=1.6, 8.0 Hz, 1H), 7.30-7.23 (m, 1H), 7.23-7.16 (m, 1H), 7.16-7.06 (m, 2H), 6.99 (td, J=2.0, 8.0 Hz, 1H), 6.86 (td, J=7.6, 1.4 Hz, 1H), 6.80-6.75 (m, 1H). LCMS (ESI): t_{R}=1.114.

4. Synthesis of compound 2-fluorophenyl 2-iodophenyl ether. To a solution of 2-fluorophenyl 2-iodophenyl ether (4.70 g, 15.0 mmol) in DMF (30 mL) were add Pin₂B₂ (7.60 g, 29.9 mmol), bis(triphenylphosphine)palladium(II) chloride (525 mg, 0.75 mmol) and potassium acetate (4.41 g, 44.9 mmol). the mixture was stirred at 120° C. for 12 h under N₂ atmosphere. The mixture was filtered, then H₂O (300 mL) was added and extracted with EtOAc. The combined organic layers were washed with brine and dried over Na₂SO₄, filtered and concentrated. The residue was purified by Prep-HPLC to give 2-(2-(2-fluorophenoxy)phenyl)-4,4,5,5-tetramethyl-1,3,2-dioxaborolane (2.20 g). ¹H NMR (400 MHz, DMSO-d₆) δ 7.69 (dd, 1.6, 7.6 Hz, 1H), 7.40-7.28 (m, 2H), 7.22 (d, J=1.2 Hz, 1H), 7.12-7.04 (m, 3H), 6.95 (d, J=8.0 Hz, 1H), 1.13 (s, 12H). LCMS (ESI): m/z, 315.2 [M+H]⁺.

5. Synthesis of compound int. 46-1. To a solution of 2,4-dichloro-5,5-dimethyl-5H,6H,7H-pyrrolo[2,3-d]pyrimidin-6-one (330 mg, 1.42 mmol) in i-PrOH (25 mL) were added 2-[2-(2-fluorophenoxy)phenyl]-4,4,5,5-tetramethyl-1,3,2-dioxaborolane (2.20 g, 7.00 mmol), pyridine (0.35 mL), cupric acetate monohydrate (340 mg, 1.71 mmol) and 4 Å MS (2.50 g), the mixture was stirred at 50° C. for 12 h with O₂ atmosphere. The mixture was filtered and the filtered cake was washed with EtOAc. The filtrate was concentrated to yield a residue which was purified by column chromatography to give 2,4-dichloro-7-(2-(2-fluorophenoxy)phenyl)-5,5-dimethyl-5,7-dihydro-6H-pyrrolo[2,3-d]pyrimidin-6-one (282 mg). ¹H NMR (400 MHz, DMSO-d₆) δ 7.58 (td, J=1.6, 7.6 Hz, 2H), 7.37 (d, J=1.2 Hz, 2H), 7.20-7.09 (m, 3H), 7.05-6.98 (m, 1H), 1.55 (s, 3H), 1.31 (s, 3H). LCMS (ESI): m/z, 418.1, 420.1 [M+H]⁺.

6. Synthesis of compound int. 46-2. Compound int. 46-2 was synthesized as compound int. 44-2. LCMS (ESI): m/z, 528.3, 530.2 [M+H]⁺.

7. Synthesis of compound int. 46-3. Compound int. 46-3 was synthesized as compound int. 44-3. LCMS (ESI): m/z, 523.3 [M+H]⁺.

8. Synthesis of compound int. 46. Compound int. 46 was synthesized as compound int. 44. ¹H NMR (400 MHz, DMSO-d₆) δ 7.39 (d, J=7.6 Hz, 2H), 7.29-7.18 (m, 2H), 7.09-6.92 (m, 4H), 6.60 (d, J=4.8 Hz, 1H), 4.68 (s, 1H), 4.16 (dd, J=1.6, 6.0, 2H), 2.85 (d, J=4.4 Hz, 3H), 1.37 (s, 3H), 1.18 (s, 3H). LCMS (ESI): m/z, 409.2 [M+H]⁺.

9. Synthesis of compound E29. Compound E29 was synthesized as compound E15. ¹H NMR (400 MHz, CDCl₃) δ 7.42-7.31 (m, 2H), 7.20 (dd, J=3.2, 1.3 Hz, 1H), 7.14-6.99 (m, 4H), 6.94 (d, J=8.0 Hz, 1H), 5.17 (dd, J=12.8, 14.8 Hz, 1H), 4.97 (dd, J=11.6, 14.8, 1H), 4.45 (d, J=8.4 Hz, 1H), 4.33 (d, J=5.2 Hz, 1H), 3.48-3.33 (m, 2H), 3.05 (d, J=4.8 Hz, 3H), 2.06-1.98 (m, 2H), 1.92-1.77 (m, 2H), 1.49 (s, 3H), 1.35 (d, J=3.2 Hz, 3H). ¹H NMR (400 MHz, DMSO-dc) δ 7.46 (dd, J=7.6, 9.6, 2H), 7.29 (dd, J=5.6, 9.2 Hz, 2H), 7.18-7.00 (m, 3H), 6.95 (d, J=5.2 Hz, 1H), 6.68 (d, J=4.8 Hz, 1H), 5.81 (s, 1H), 4.90 (d, J=12.8 Hz, 2H), 4.53 (d, J=4.2 Hz, 1H), 3.29 (s, 2H), 2.86 (d, J=4.4 Hz, 3H), 1.93-1.73 (m, 4H), 1.42 (s, 3H), 1.23-1.20 (m, 3H). LCMS (ESI): m/z, 573.3 [M+H]⁺.

### Example 107. Preparation of Compound E30

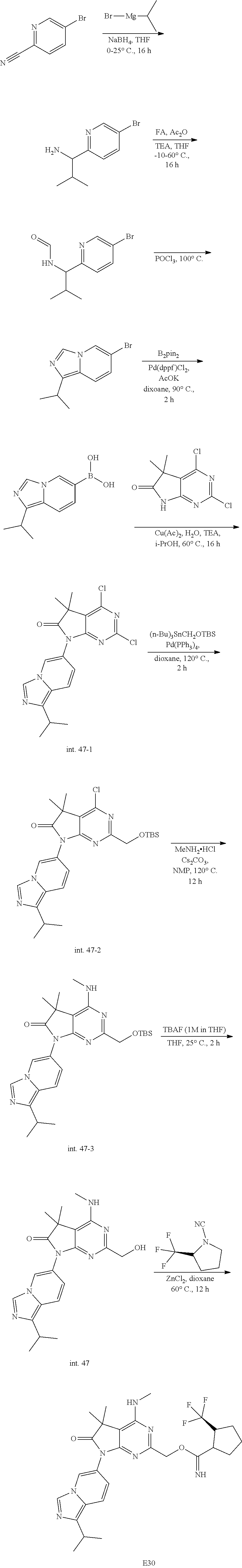

1. Synthesis of compound 1-(5-bromopyridin-2-yl)-2-methylpropan-1-amine. To solution of 5-bromopicolinonitrile (5.00 g, 27.3 mmol) in THF (1 mL), i-PrMgBr (55 mL, 1 M) was added at 0° C. under N₂ atmosphere. The resulting solution was stirred for 16 h at 0° C., MeOH (1 mL) and NaBH₄ (3.10 g, 82.0 mmol) were added at 0° C. The resulting solution was stirred for 16 h at 25° C. The reaction mixture was extracted with EA. The organic layers were washed with brine, dried over Na₂SO₄, filtered and concentrated. The residue was purified by column chromatography to afford 1-(5-bromopyridin-2-yl)-2-methylpropan-1-amine (1.94 g). ¹H NMR (400 MHz, DMSO-d₆) δ 8.60-8.57 (m, 2H), 7.95 (dd, J=2.4, 8.4 Hz, 2H), 7.38 (d, J=8.4 Hz, 2H), 3.58 (d, J=6.4 Hz, 2H), 1.91-1.83 (m, 2H), 0.80 (d, J=6.8 Hz, 6H), 0.75 (d, J=6.8 Hz, 6H). LCMS (ESI): m/z, 229.0 [M+H]⁺.

2. Synthesis of compound N-(1-(5-bromopyridin-2-yl)-2-methylpropyl)formamide. A mixture of formic acid (2 mL) and Ac₂O (3 mL) was stirred at 60° C. for 4 h, then cooled to ambient temperature. The resulting mixture was added to a solution of 1-(5-bromopyridin-2-yl)-2-methylpropan-1-amine (1.84 g, 8.03 mmol) and TEA (0.78 mL) in THF (56 mL) at −10° C. The reaction mixture was stirred while allowing to warm to ambient temperature, and stirred at ambient temperature for 16 h. The mixture was poured into aqueous saturated NaHCO₃ and extracted with EA. The organic layers were washed with brine, dried over Mg₂SO₄, filtered, and concentrated to give N-(1-(5-bromopyridin-2-yl)-2-methylpropyl)formamide (2.24 g crude). ¹H NMR (400 MHz, DMSO-d₆) δ 8.72 (d, J=2.4 Hz, 1H), 8.60 (d, J=9.2 Hz, 1H), 8.17-8.14 (m, 1H), 8.08 (dd, J=2.8, 8.4 Hz, 1H), 7.38 (d, J=8.4 Hz, 1H), 4.81 (dd, J=7.2, 9.2 Hz, 1H), 2.19-2.13 (m, 1H), 0.90 (d, J=6.8 Hz, 4H), 0.82 (d, J=6.8 Hz, 4H). LCMS (ESI): m/z, 257.0 [M+H]⁺.

3. Synthesis of compound 6-bromo-1-isopropylimidazo[1,5-a]pyridine. A mixture of N-[1-(5-bromopyridin-2-yl)-2-methylpropyl]formamide (2.14 g, 8.32 mmol) and POCl₃ (0.85 mL) was stirred at 100° C. for 1 h under N₂ atmosphere. The mixture was added carefully NaHCO₃ and DCM. The product was extracted into EA and the organic layers were washed with brine, dried over Na₂SO₄, filtered and concentrated to give 6-bromo-1-isopropylimidazo[1,5-a]pyridine (1.85 g). ¹H NMR (400 MHz, DMSO-d₆) δ 8.57-8.56 (m, 1H), 8.20 (s, 1H), 7.54 (d, J=9.6 Hz, 1H), 6.69 (dd, J=1.6, 9.6 Hz, 1H), 3.33-3.27 (m, 1H), 1.25 (d, J=6.8 Hz, 6H). LCMS (ESI): m/z, 239.0 [M+H]⁺.

4. Synthesis of compound (1-isopropylimidazo[1,5-a]pyridin-6-yl)boronic acid. A mixture of 6-bromo-1-(propan-2-yl)imidazo[1,5-a]pyridine (930 mg, 3.89 mmol), Pin₂B₂ (1.98 g, 7.78 mmol), AcOK (954 mg, 9.72 mmol) and Pd(dppf)₂Cl₂ (307 mg, 0.39 mmol) in 1,4-dioxane (1 mL) was stirred at 100° C. for 2 h under N₂ atmosphere. The mixture was concentrated. The residue was purified by column chromatography to afford (1-isopropylimidazo[1,5-a]pyridin-6-yl)boronic acid (600 mg). LCMS (ESI): m/z, 205.0 [M+H]⁺.

5. Synthesis of compound int. 47-1. Compound int. 47-1 was synthesized as compound int. 33-1. LCMS (ESI): m/z, 390.0 [M+H]⁺.

6. Synthesis of compound int. 47-2. Compound int. 47-2 was synthesized as compound int. 43-2. LCMS (ESI): m/z, 500.3 [M+H]⁺.

7. Synthesis of compound int. 47-3. Compound int. 47-3 was synthesized as compound int. 33. LCMS (ESI): m/z, 595.3 [M+H]⁺.

8. Synthesis of compound int. 47. Compound int. 47 was synthesized as compound int. 34. LCMS (ESI): m/z, 381.2 [M+H]⁺.

9. Synthesis of compound E30. Compound E30 was synthesized as compound E16. LCMS (ESI): m/z, 545.2 [M+H]⁺.

### Example 108. Preparation of Compound E31

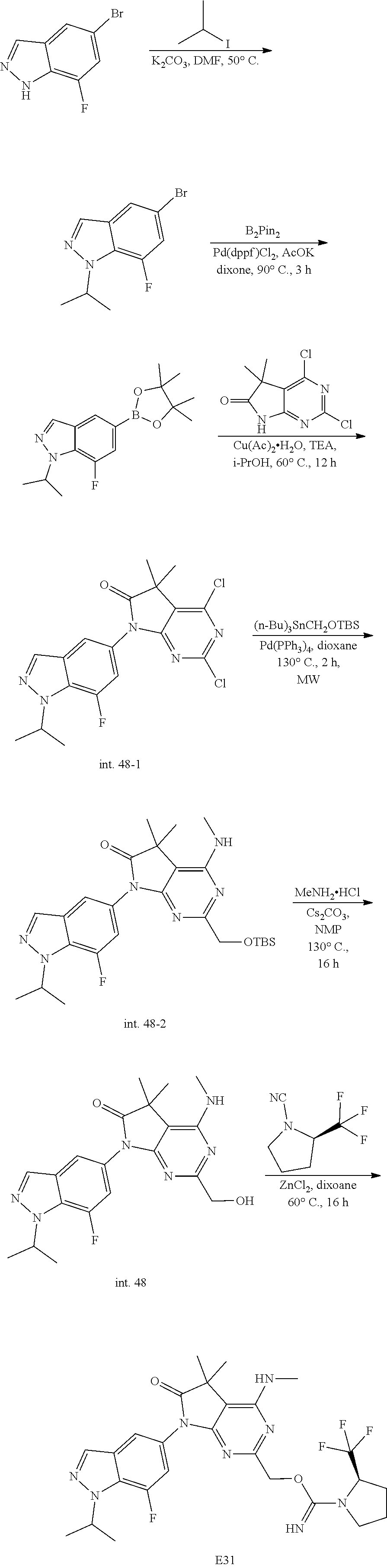

1. Synthesis of compound 5-bromo-7-fluoro-1-isopropyl-1H-indazole. To a solution of 5-bromo-7-fluoro-1H-indazole (950 mg, 4.42 mmol) and 2-iodopropane (976 mg, 5.74 mmol) in DMF (100 mL) was added K₂CO₃ (854 mg, 6.19 mmol). The mixture was stirred at 50° C. for 12 h. The mixture was diluted with EA, washed with sat.aq. NaCl, dried over Na₂SO₄, filtered and concentrated. The residue was purified by column chromatography to afford 5-bromo-7-fluoro-1-isopropyl-1H-indazole (800 mg). ¹H NMR (400 MHz, CDCl₃) δ 7.93 (d, J=2.4 Hz, 1H), 7.63 (d, J=1.2 Hz, 1H), 7.16 (dd, J=1.6, 11.6 Hz, 1H), 5.11-5.05 (m, I H), 1.58 (d, J=6.8 Hz, 6H). LCMS (ESI): m/z, 257.1, 259.0 [M+H]⁺.

2. Synthesis of compound 7-fluoro-1-isopropyl-5-(4,4,5,5-tetramethyl-1,3,2-dioxaborolan-2-yl)-1H-indazole. A mixture of 5-bromo-7-fluoro-1-isopropyl-indazole (850 mg, 3.31 mmol), 4,4,5,5-tetramethyl-2-(4,4,5,5-tetramethyl-1,3,2-dioxaborolan-2-yl)-1,3,2-dioxaborolane (1.26 g, 4.96 mmol), Pd(dppf)Cl₂ (290 mg, 0.396 mmol), and KOAc (648 mg, 6.61 mmol) in dioxane (20 mL) was stirred at 90° C. for 3 h under N₂ atmosphere. The mixture was concentrated. The residue was purified by column chromatography to afford 7-fluoro-1-isopropyl-5-(4,4,5,5-tetramethyl-1,3,2-dioxaborolan-2-yl)-1H-indazole (800 mg). LCMS (ESI): m/z, 305.3 [M+H]⁺.

3. Synthesis of compound int. 48-1. A mixture of 7-fluoro-1-isopropyl-5-(4,4,5,5-tetramethyl-1,3,2-dioxaborolan-2-yl)-1H-indazole (1.20 g, 5.17 mmol), 7-fluoro-1-isopropyl-5-(4,4,5,5-tetramethyl-1,3,2-dioxaborolan-2-yl)indazole (2.83 g, 9.31 mmol), Cu(OAc)₂·H₂O (1.03 g, 5.17 mmol), TEA (1.44 mL), and (2R,3S)-2-[[(2R)-2-acetamido-3-(3-fluorophenyl)propanoyl]amino]-5-methoxy-4-methoxycarbonyl-3-methyl-5-oxo-pentanoic acid (4.55 g, 10.3 mmol) in i-PrOH (20 mL) was stirred at 60° C. for 12 h under 02 atmosphere. The mixture was filtered and the filtrate was concentrated. The residue was purified by column chromatography to afford 2,4-dichloro-7-(7-fluoro-1-isopropyl-1H-indazol-5-yl)-5,5-dimethyl-5,7-dihydro-6H-pyrrolo[2,3-d]pyrimidin-6-one (1.2 g). ¹H NMR (400 MHz, CDCl₃) (8.08 (d, J=2.0 Hz, 1H), 7.59 (d, J=1.6 Hz, 1H), 7.11 (dd, J=2.0, 12.4 Hz, 1H), 5.19-5.12 (m, 1H), 1.65 (s, 6H), 1.61 (d, J=6.8 Hz, 6H). LCMS (ESI): m/z, 408.1 [M+H]⁺.

4. Synthesis of compound int. 48-2. Compound int. 48-2 was synthesized as compound int. 44-2. LCMS (ESI): m/z, 518.2 [M+H]⁺.

5. Synthesis of compound int. 48. Compound int. 48 was synthesized as compound int. 33. LCMS (ESI): m/z, 399.2 [M+H]⁺.

6. Synthesis of compound E31. Compound E31 was synthesized as compound E16. ¹H NMR (400 MHz, CDCl₃) δ 8.03 (d, J=2.0 Hz, 1H), 7.62 (d, J=2.0 Hz, 1H), 7.21 (dd, J=1.6, 12.8 Hz, 1H), 5.21-5.09 (m, 2H), 5.06-5.02 (m, 2H), 4.50-4.38 (m, 2H), 3.47-3.45 (m, 2H), 3.07 (d, J=4.8 Hz, 3H), 2.07-2.01 (m, I H), 1.98-1.95 (m, I H), 1.87-1.81 (m, 1H), 1.77-1.66 (m, 1H), 1.59 (d, J=6.4 Hz, 6H), 1.50 (s, 6H). LCMS (ESI): m/z, 563.3 [M+H]⁺.

### Example 109. Preparation of Compound E32

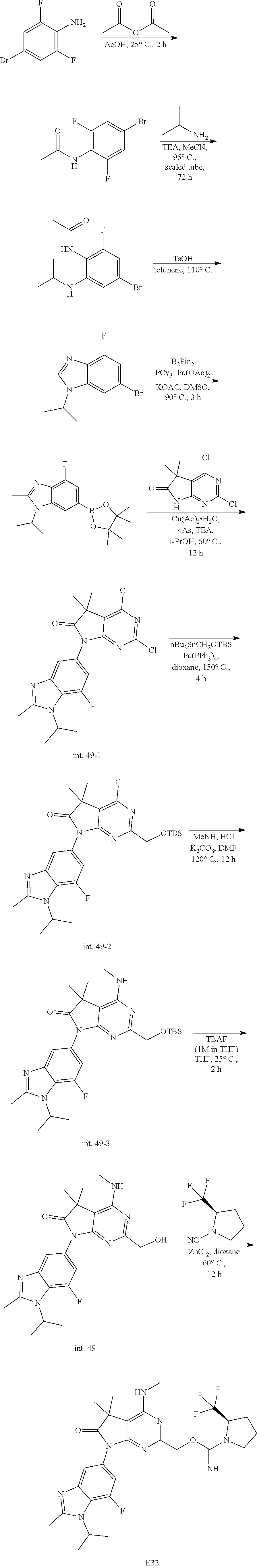

1. Synthesis of compound N-(4-bromo-2,6-difluorophenyl)acetamide. To a solution of 4-bromo-2,6-difluoro-aniline (6.0 g, 28.8 mmol) in acetic acid (60 mL) was added acetyl acetate (18 mL) dropwise. The mixture was stirred at 25° C. for 2 h. The mixture was added into ice water, the precipitate was filtered. and the cake was dissolved with DCM, washed with sat.aq. NaHCO₃, dried over Na₂SO₄, filtered and concentrated to afford N-(4-bromo-2,6-difluorophenyl)acetamide (6.2 g). ¹H NMR (400 MHz, DMSO-d₆) δ 9.76 (s, 1H), 7.53 (d, J=7.2 Hz, 2H), 2.05 (s, 3H). LCMS (ESI): m/z, 249.9, 251.9 [M+H]⁺.

2. Synthesis of compound N-(4-bromo-2-fluoro-6-(isopropylamino)phenyl)acetamide. To a solution of N-(4-bromo-2,6-difluorophenyl)acetamide (6.2 g, 24.8 mmol) and propan-2-amine (1.61 g, 27.3 mmol) in ACN (32 mL) was added TEA (6.89 mL). The mixture was stirred at 95° C. for 72 h in sealed tube. The mixture was concentrated to afford N-(4-bromo-2-fluoro-6-(isopropylamino)phenyl)acetamide (5.0 g, 60% purity). LCMS (ESI): m/z, 289.1, 291.1 [M+H]⁺.

3. Synthesis of compound 6-bromo-4-fluoro-1-isopropyl-2-methyl-1H-benzo[d]imidazole. To a solution of N-(4-bromo-2-fluoro-6-(isopropylamino)phenyl)acetamide (4.5 g, 7.78 mmol) in toluene (82 mL) was added 4-methylbenzenesulfonic acid (2.50 mL). The mixture was stirred at 110° C. for 16 h. The mixture was concentrated. The residue was purified by column chromatography to afford 6-bromo-4-fluoro-1-isopropyl-2-methyl-1H-benzo[d]imidazole (1.9 g). LCMS (ESI): m/z, 271.0, 273.0 [M+H]⁺.

4. Synthesis of compound 4-fluoro-1-isopropyl-2-methyl-6-(4,4,5,5-tetramethyl-1,3,2-dioxaborolan-2-yl)-1H-benzo[d]imidazole. A mixture of 6-bromo-4-fluoro-1-isopropyl-2-methyl-1H-benzo[d]imidazole (3.2 g, 11.8 mmol), 4,4,5,5-tetramethyl-2-(4,4,5,5-tetramethyl-1,3,2-dioxaborolan-2-yl)-1,3,2-dioxaborolane (4.50 g, 17.7 mmol), diacetoxypalladium (264 mg, 1.18 mmol), tricyclohexylphosphane (662 mg, 2.36 mmol), and KOAc (3.47 g, 35.4 mmol) in DMSO (30 mL) was stirred at 90° C. for 3 h. The mixture was concentrated. The residue was purified by column chromatography to afford 4-fluoro-1-isopropyl-2-methyl-6-(4,4,5,5-tetramethyl-1,3,2-dioxaborolan-2-yl)-1H-benzo[d]imidazole (2.6 g). LCMS (ESI): m/z, 237.1 [M+H]⁺

5. Synthesis of compound E32. Other steps of compound E32 were synthesized as compound E22. ¹H NMR (400 MHz, DMSO-d₆) δ 7.59 (d, J=2.0 Hz, ¹H), 7.04 (dd, J=2.0, 11.2 Hz, ¹H), 6.73-6.72 (m, ¹H), 5.83 (brs, 1H), 5.02-4.89 (m, 2H), 4.81-4.74 (m, ¹H), 4.51-4.47 (m, ¹H), 3.27-3.25 m, 2H), 2.90 (d, J=4.4 Hz, 3H), 2.61 (m, 3H), 1.77-1.74 (m, 2H), 1.62-1.58 (m, 2H), 1.53 (d, J=6.8 Hz, 6H), 1.46 (s, 6H). LCMS (ESI): m/z, 577.3 [M+H]⁺.

### Example 110. Preparation of Compound E33

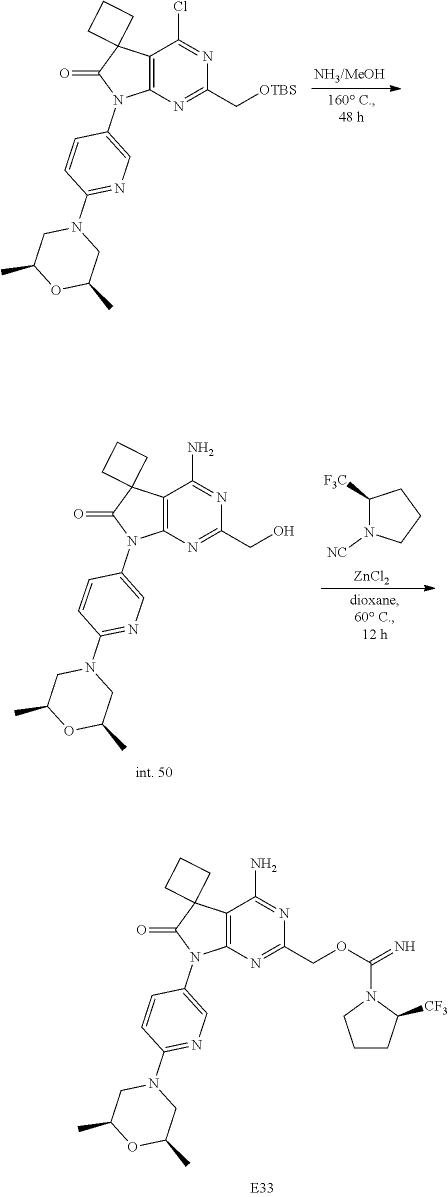

1. Synthesis of compound int. 50. A solution of 2′-(((tert-butyldimethylsilyl)oxy)methyl)-4′-chloro-7′-(6-((2S,6R)-2,6-dimethylmorpholino)pyridin-3-yl)spiro[cyclobutane-1,5′-pyrrolo[2,3-d]pyrimidin]-6′(7′H)-one (400 mg, 0.74 mmol) in NH₃ (8 mL, 7 M in MeOH) was stirred at 160° C. for 48 h. The mixture was concentrated. The residue was purified by column chromatography to give compound 4′-amino-7′-(6-((2S,6R)-2,6-dimethylmorpholino)pyridin-3-yl)-2′-(hydroxymethyl)spiro[cyclobutane-1,5′-pyrrolo[2,3-d]pyrimidin]-6′(7′H)-one (61 mg). LCMS (ESI): m/z, 411.3 [M+H]⁺.

2. Synthesis of compound E33. Compound E33 was synthesized as compound E16. LCMS (ESI): m/z, 575.3 [M+H]⁺.

### Example 111. Preparation of Compound E34

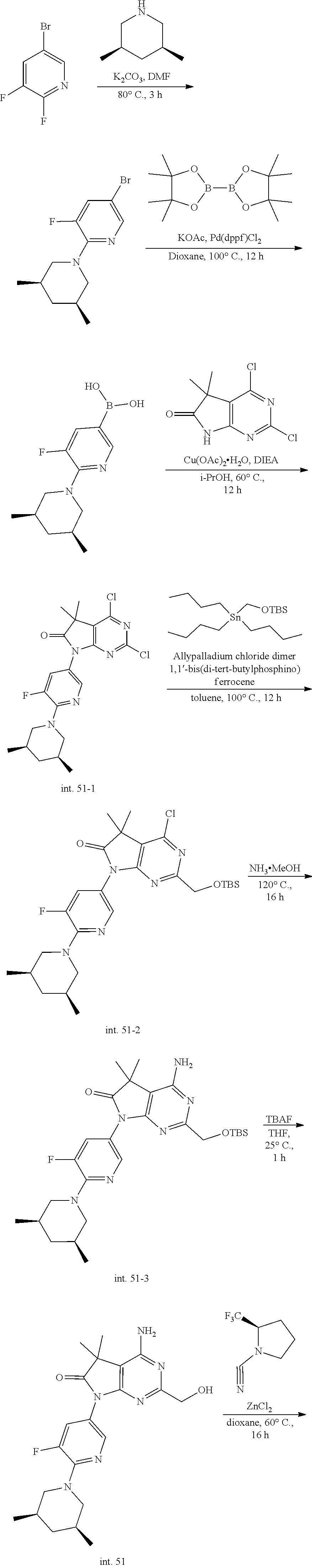

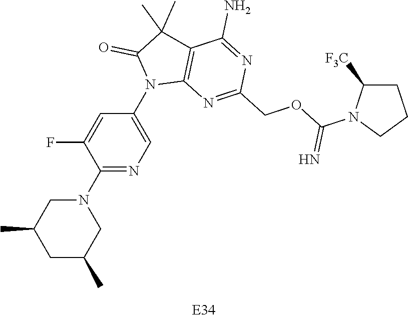 [SMILES: C[C@@H]1C[C@H](C)CN(c2ncc(N3C(=O)C(C)(C)c4c(N)nc(COC(=N)N5CCC[C@@H]5C(F)(F)F)nc43)cc2F)C1]

1. Synthesis of compound 5-bromo-2-((3R,5S)-3,5-dimethylpiperidin-1-yl)-3-fluoropyridine. A mixture of 5-bromo-2,3-difluoropyridine (500 mg, 2.58 mmol), (3R,5S)-3,5-dimethylpiperidine (292 mg, 2.58 mmol) and K₂CO₃ (713 mg, 5.16 mmol) in DMF (10 mL) was stirred at 80° C. for 3 h. The mixture was filtered and concentrated. The residue was purified by column chromatography to give compound 5-bromo-2-((3R,5S)-3,5-dimethylpiperidin-1-yl)-3-fluoropyridine (688 mg). LCMS (ESI): m/z, 287.1 [M+H]⁺.

2. Synthesis of compound (6-((3R,5S)-3,5-dimethylpiperidin-1-yl)-5-fluoropyridin-3-yl)boronic acid. A mixture of 4,4,4′,4′,5,5,5′,5′-octamethyl-2,2′-bi(1,3,2-dioxaborolane) (730 mg, 2.87 mmol), 5-bromo-2-((3R,5S)-3,5-dimethylpiperidin-1-yl)-3-fluoropyridine (688 mg, 2.40 mmol), KOAC (588 mg, 5.99 mmol) and Pd(dppf)Cl₂ (175 mg, 0.24 mmol) in 1,4-dioxane (15 mL) was stirred at 100° C. for 12 h under N₂ atmosphere. The mixture was filtered and concentrated. The residue was purified by column chromatography to give compound (6-((3R,5S)-3,5-dimethylpiperidin-1-yl)-5-fluoropyridin-3-yl)boronic acid (800 mg crude). LCMS (ESI): m/z, 253.1 [M+H]⁺.

3. Synthesis of compound int. 51-1. A mixture of 2,4-dichloro-5,5-dimethyl-5,7-dihydro-6H-pyrrolo[2,3-d]pyrimidin-6-one (295 mg, 1.27 mmol), (6-((3R,5S)-3,5-dimethylpiperidin-1-yl)-5-fluoropyridin-3-yl)boronic acid (800 mg, 3.17 mmol), TEA (385 mg, 3.81 mmol), Cu(OAc)₂·H₂O (253 mg, 1.27 mmol) and 4As (1.12 g) in i-PrOH (12 mL) was stirred at 60° C. for 12 h under O₂ atmosphere. The mixture was filtered and concentrated. The residue was purified by column chromatography to give compound 2,4-dichloro-7-(6-((3S,5R)-3,5-dimethylpiperidin-1-yl)-5-fluoropyridin-3-yl)-5,5-dimethyl-5,7-dihydro-6H-pyrrolo[2,3-d]pyrimidin-6-one (188 mg). LCMS (ESI): m/z, 438.2 [M+H]⁺. [00797]4. Synthesis of compound int. 51-2. A mixture of 2,4-dichloro-7-(6-((3S,5R)-3,5-dimethylpiperidin-1-yl)-5-fluoropyridin-3-yl)-5,5-dimethyl-5,7-dihydro-6H-pyrrolo[2,3-d]pyrimidin-6-one (138 mg, 0.31 mmol), Bu₃SnCH₂OTBS (151 mg, 0.35 mmol), 1,1′-bis(di-tert-butylphosphino)ferrocene (12 mg, 0.03 mmol) and allylpalladium chloride dimer (5 mg, 0.13 mmol) in toluene (3 mL) was stirred at 100° C. for 16 h under N₂ atmosphere. The mixture was filtered and concentrated. The residue was purified by column chromatography to give compound 2-(((tert-butyldimethylsilyl)oxy)methyl)-4-chloro-7-(6-((3S,5R)-3,5-dimethylpiperidin-1-yl)-5-fluoropyridin-3-yl)-5,5-dimethyl-5,7-dihydro-6H-pyrrolo[2,3-d]pyrimidin-6-one (218 mg). LCMS (ESI): m/z, 548.4 [M+H]⁺.

5. Synthesis of compound int. 51-3. A solution of 2-(((tert-butyldimethylsilyl)oxy)methyl)-4-chloro-7-(6-((3S,5R)-3,5-dimethylpiperidin-1-yl)-5-fluoropyridin-3-yl)-5,5-dimethyl-5,7-dihydro-6H-pyrrolo[2,3-d]pyrimidin-6-one (178 mg, 0.32 mmol) in NH₃ (5 mL, 7 M in MeOH) was stirred at 120° C. for 16 h. The mixture was concentrated to give title compound (172 mg crude). LCMS (EST): m/z, 529.4 [M+H]⁺.

6. Synthesis of compound int. 51. To a solution of 4-amino-2-(((tert-butyldimethylsilyl)oxy)methyl)-7-(6-((3S,5R)-3,5-dimethylpiperidin-1-yl)-5-fluoropyridin-3-yl)-5,5-dimethyl-5,7-dihydro-6H-pyrrolo[2,3-d]pyrimidin-6-one (172 mg, 0.33 mmol) in THF (4 mL) was dropwise added TBAF (0.65 mL, 1 M in THF) at 0° C., and the mixture was stirred at 25° C. for 1 h. The mixture was diluted with EtOAc, washed with water and brine, dried over Na₂SO₄, filtered and concentrated. The residue was purified by column chromatography to give title compound (40 mg). LCMS (ESI): m/z, 415.3 [M+H]⁺.

7. Synthesis of compound E34. Compound E34 was synthesized as compound E16. ¹H NMR (400 MHz, CDCl₃) δ 7.23 (d, J=2.0 Hz, 1H), 7.13 (dd, J=8.4, 2.2 Hz, 1H), 6.82 (d, J=8.4 Hz, 1H), 5.22 (d, J=14.4 Hz, 1H), 5.05 (d, J=14.4 Hz, 1H), 4.61 (t, J=8.8 Hz, 2H), 4.54-4.42 (m, 2H), 3.54-3.41 (m, 2H), 3.24 (t, J=8.8 Hz, 2H), 3.07 (d, J=4.8 Hz, 3H), 2.08-1.83 (m, 4H), 1.48 (s, 6H). LCMS (ESI): m/z, 579.3 [M+H]⁺.

### Example 112. Preparation of Compound E35

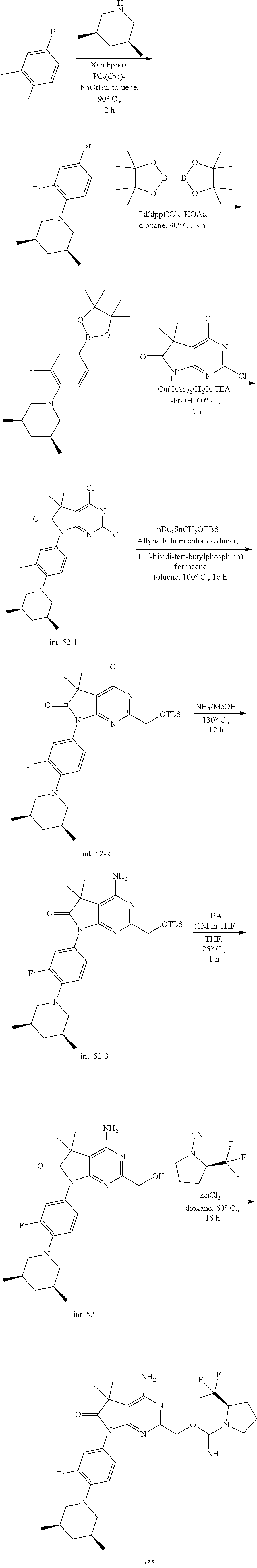

1. Synthesis of compound (3R,5S)-1-(4-bromo-2-fluorophenyl)-3,5-dimethylpiperidine. A mixture of (3R,5S)-3,5-dimethylpiperidine (600 mg, 5.30 mmol), 1-bromo-3-fluoro-4-iodobenzene (2.39 g, 7.95 mmol), XantPhos (306 mg, 0.530 mmol), Pd₂(dba)₃ (242 mg, 0.265 mmol), t-BuONa (1.27 g, 13.25 mmol) in PhMe (50 mL) was stirred at 90° C. for 2 h under N₂ atmosphere. The mixture was filtered, and the filtrate was concentrated. The residue was purified by column chromatography to afford (3R,5S)-1-(4-bromo-2-fluorophenyl)-3,5-dimethylpiperidine (1.40 g). LCMS (ESI): m/z, 286.1 [M+H]⁺.

2. Synthesis of compound E35. Other steps of compound E35 were synthesized as compound E34. ¹H NMR (400 MHz, DMSO-d₆) b 7.23 (dd, J=2.4, 13.6 Hz, Hz, 1H), 7.15 (dd, J=2.4, 8.4 Hz, Hz, 1H), 7.10-7.05 (m, 1H), 6.78 (brs, 2H), 5.78 (brs, 1H), 5.03-4.84 (m, 2H), 4.54-4.50 (m, 1H), 3.32-3.27 (m, 4H), 2.25-2.18 (m, 2H), 1.88-1.77 (m, 7H), 1.42 (s, 6H), 0.89 (d, J=7.6 Hz, 6H), 0.73-0.64 (m, 1H). LCMS (ESI): m/z, 578.3 [M+H]⁺.

### Example 113. Preparation of Compound E36

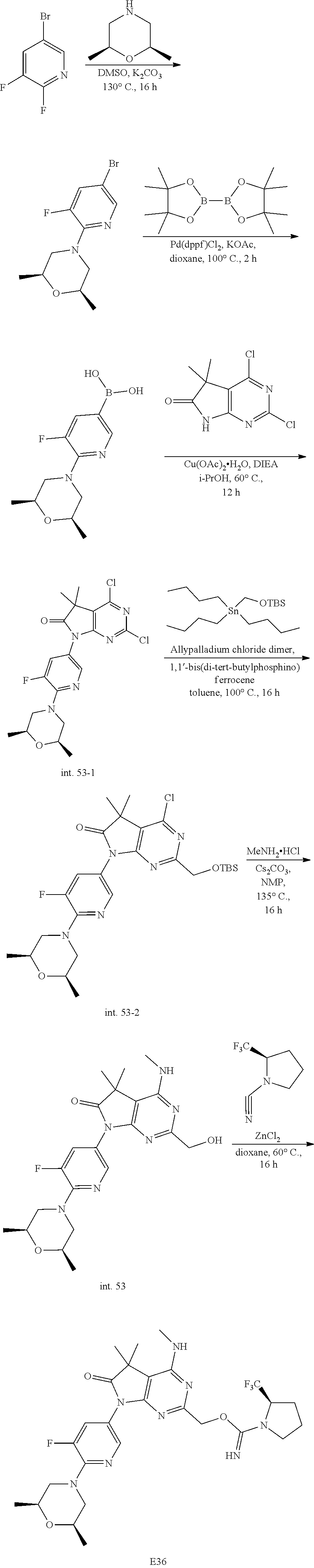

Compound E36 was synthesized as compound E34. ¹H NMR (400 MHz, DMSO-d₆) δ 8.11 (d, J=2.0 Hz, 1H), 7.71 (dd, J=2.4, 14.4 Hz, 1H), 6.84 (s, 2H), 5.02-4.84 (m, 2H), 4.57-4.53 (m, 1H), 3.89 (dd, J=1.6, 12.4 Hz, 2H), 3.73-3.65 (m, 2H), 3.31-3.26 (m, 2H), 2.62-2.54 (m, 2H), 2.08-1.61 (m, 5H), 1.42 (s, 6H), 1.13 (d, J=6.4 Hz, 6H). LCMS (ESI): m/z, 595.4 [M+H]⁺.

### Example 114. Preparation of Compound E37

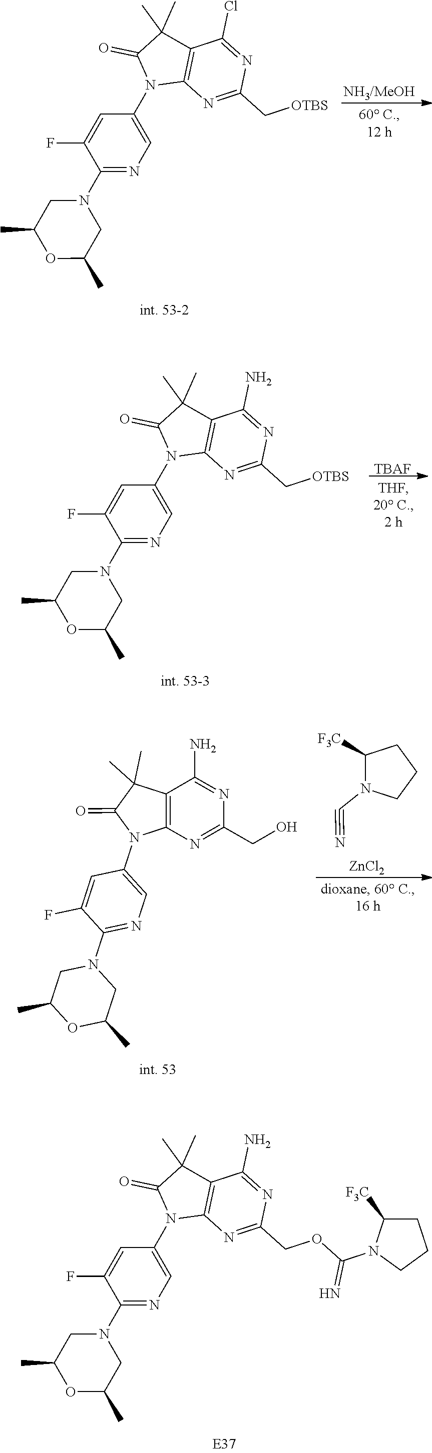

1. Synthesis of compound int. 53-3. Compound int. 53-3 was synthesized as compound int. 51-3. LCMS (ESI): 531.3 [M+H].

2. Synthesis of compound int. 53a. A mixture of 4-amino-2-(((tert-butyldimethylsilyl)oxy)methyl)-7-(6-((2R,6S)-2,6-dimethylmorpholino)-5-fluoropyridin-3-yl)-5,5-dimethyl-5,7-dihydro-6H-pyrrolo[2,3-d]pyrimidin-6-one (483 mg, 50.0% purity, 0.45 mmol) and TBAF (0.9 mL, 1 M) in THF (4 mL) was stirred at 20° C. for 2 h under N₂ atmosphere. Water was added to the reaction mixture, and it was extracted with EtOAc. The combined organic phase was washed with brine, dried over Na₂SO₄, filtered and concentrated. The residue was purified by column chromatography to afford 4-amino-7-(6-((2R,6S)-2,6-dimethylmorpholino)-5-fluoropyridin-3-yl)-2-(hydroxymethyl)-5,5-dimethyl-5,7-dihydro-6H-pyrrolo[2,3-d]pyrimidin-6-one (190 mg). LCMS (ESI): m/z, 417.1 [M+H]⁺.

3. Synthesis of compound E37. Compound E37 was synthesized as compound E16. ¹H NMR (400 MHz, DMSO-d₆) δ 8.11 (d, J=2.0 Hz, 1H), 7.71 (dd, J=2.4, 14.4 Hz, 1H), 6.84 (s, 2H), 5.02-4.84 (m, 2H), 4.54-4.50 (m, 1H), 3.89 (dd, J=1.6, 12.4 Hz, 2H), 3.73-3.65 (m, 2H), 3.31-3.26 (m, 2H), 2.62-2.54 (m, 2H), 2.08-1.61 (m, 5H), 1.42 (s, 6H), 1.13 (d, J=6.2 Hz, 6H). LCMS (ESI): m/z, 495.3 [M+H]⁺.

### Example 115. Preparation of Compound E38

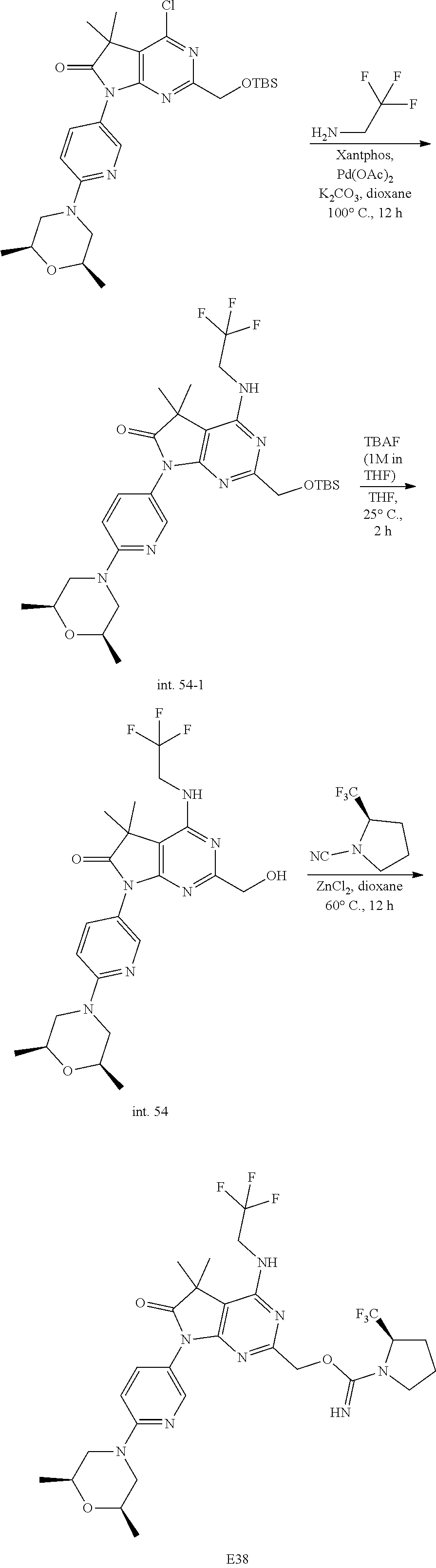

1. Synthesis of compound int. 54-1. Compound int. 54-1 was synthesized as compound int 39-4. ¹H NMR (400 MHz, DMSO-d₆) δ 8.11 (d, J=2.8 Hz, 1H), 7.58-7.55 (m, 1H), 7.19 (t, J=6.4 Hz, 1H), 6.93 (d, J=9.2 Hz, 1H), 4.48 (s, 2H), 4.34-4.28 (m, 2H), 4.20-4.16 (m, 2H), 3.64-3.59 (m, 2H), 2.46-2.41 (m, 2H), 1.46 (s, 6H), 1.19-1.16 (m, 6H), 0.84 (s, 9H). LCMS (ESI): m/z, 595.3 [M+H]⁺.

2. Synthesis of compound int. 54. Compound int. 54 was synthesized as compound int 34. LCMS (ESI): m/z, 481.1 [M+H]⁺.

3. Synthesis of compound E38. Compound E38 was synthesized as compound E16. ¹H NMR (400 MHz, CDCl₃) δ 8.28 (d, J=2.8 Hz, 1H), 7.58 (d, J=8.8 Hz, 2.4 Hz, 1H), 6.68 (d, J=9.2 Hz, 1H), 5.23-5.06 (m, 2H), 4.56 (t, J=6.8 Hz, 1H), 4.46-4.42 (m, 1H), 4.35-4.26 (m, 2H), 4.11-4.07 (m, 2H), 3.74-3.68 (m, 2H), 3.50-3.41 (m, 2H), 2.59-2.53 (m, 2H), 2.11-2.06 (m, 2H), 1.97-1.92 (m, 2H), 1.51 (s, 6H), 1.27 (d, J=6.4 Hz, 6H). LCMS (ESI): m/z, 645.4 [M+H]⁺.

### Example 116. Preparation of Compound E39

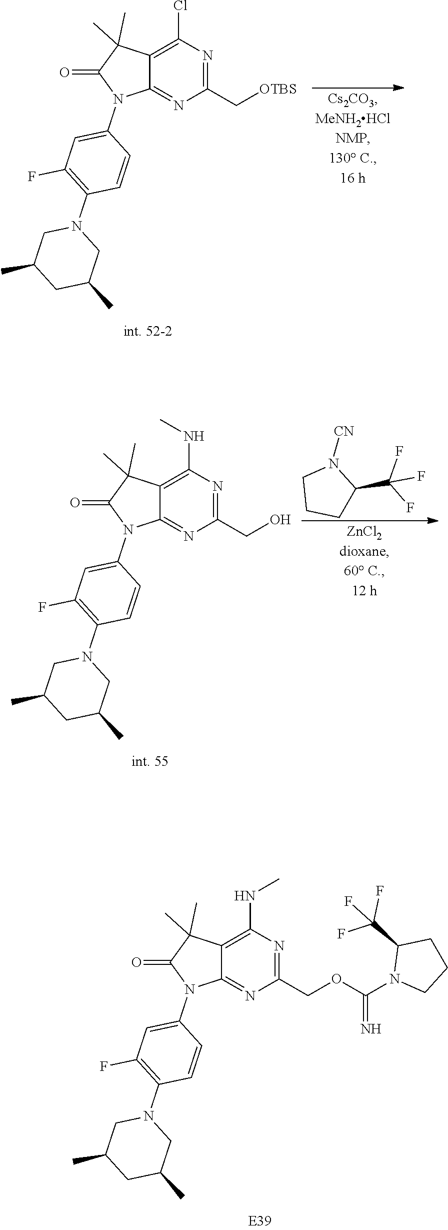

Compound E39 was synthesized as compound E17. ¹H NMR (400 MHz, DMSO-d₆) δ 7.25-7.05 (m, 3H), 6.71-6.69 (m, 1H), 5.76 (s, 1H), 5.05-4.91 (m, 2H), 4.58-4.54 (m, 1H), 2.90 (d, J=4.4 Hz, 3H), 2.25-2.18 (m, 2H), 1.92-1.77 (m, 7H), 0.88 (d, J=6.0 Hz, 6H), 0.73-0.63 (m, 1H). LCMS (ESI): m/z, 592.3 [M+H]⁺.

### Example 118. Preparation of Compound E41

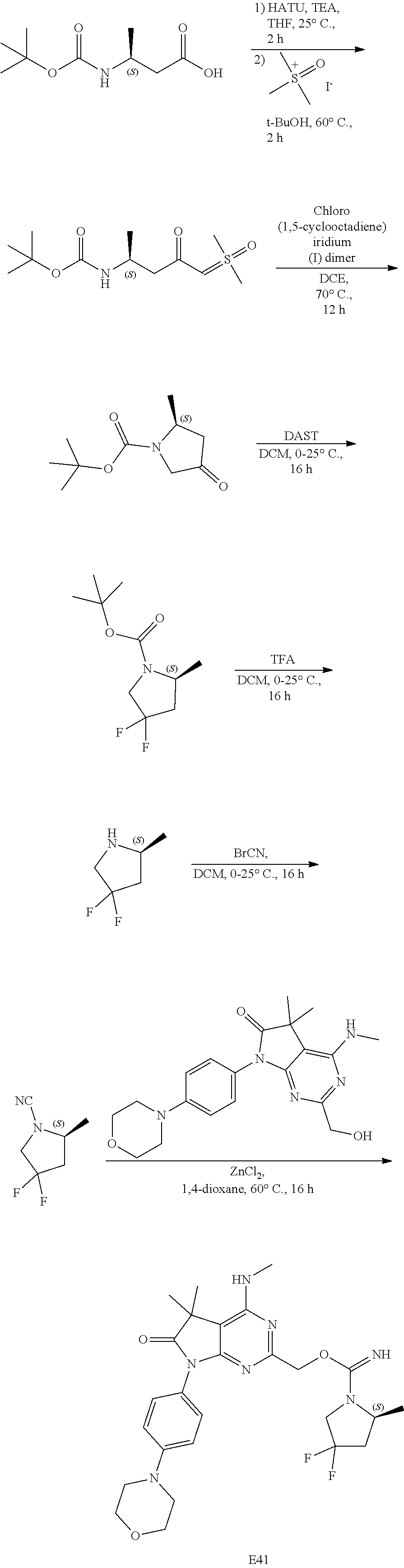

1. Synthesis of compound tert-butyl N-[(1S)-4-[dimethyl(oxo) sulfanylidene]-1-methyl-3-oxo-butyl]carbamate. A suspension of (3S)-3-(tert-butoxycarbonylamino)butanoic acid (5.00 g, 24.6 mmol) and HATU (10.3 g, 27.1 mmol) in THF (50 mL) was treated with TEA (13.7 mL), and the resulting solution was stirred at 25° C. for 16 h. The mixture was added dropwise at 0° C. to a mixture of t-BuOK (1 M, 88.57 mL) and BLAH methane iodide (19.5 g, 88.6 mmol) in THF (50 mL), which was heated to 60° C. for 2 h, then cooled in ice-water bath for 15 min before the mixture was added. The resulting reaction mixture was further stirred at 25° C. for 3 h. Water (100 mL) was added and the mixture was extracted with DCM. The combined organic phases were washed with brine, dried over Na₂SO₄, filtered and concentrated. The residue was purified by column chromatography to give tert-butyl N-[(1S)-4-[dimethyl(oxo) sulfanylidene]-1-methyl-3-oxo-butyl]carbamate (4.8 g). 1H NMR (400 MHz, CDCl₃) δ 5.45 (s, 1H), 4.43 (d, J=4.8 Hz, 1H), 3.96 (s, 1H), 3.59-3.29 (m, 6H), 2.44-2.23 (m, 2H), 1.44 (s, 9H), 1.18 (d, J=6.4 Hz, 3H).

2. Synthesis of compound tert-butyl (2S)-2-methyl-4-oxo-pyrrolidine-1-carboxylate. To a solution of tert-butyl N-[(1S)-4-[dimethyl(oxo)-sulfanylidene]-1-methyl-3-oxo-butyl]carbamate (4.8 g, 17.3 mmol) in DCE (50 mL) was added chloro(1,5-cyclooctadiene)iridium(I) dimer (0.3 g, 0.45 mmol). The mixture was stirred at 70° C. for 16 h under N₂ atmosphere. The mixture was concentrated. The residue was purified by column chromatography to give tert-butyl (2S)-2-methyl-4-oxo-pyrrolidine-1-carboxylate (3.0 g). ¹H NMR (400 MHz, CDCl₃) δ 5.45 (s, 1H), 4.43 (d, J=4.8 Hz, 1H), 3.96 (s, 1H), 3.59-3.29 (m, 6H), 2.44-2.23 (m, 2H), 1.59-1.35 (m, 9H), 1.30-1.06 (m, 3H).

3. Synthesis of compound tert-butyl (2S)-4,4-difluoro-2-methyl-pyrrolidine-1-carboxylate. To a solution of tert-butyl (2S)-2-methyl-4-oxo-pyrrolidine-1-carboxylate (3.0 g, 15.1 mmol) in DCM (30 mL) was added DAST (5.53 mL) at 0° C. dropwise. The mixture was stirred at 25° C. for 16 h. The mixture was quenched by sat.aq. NaHCO₃ and adjusted pH to 8. Water was added, and the mixture was extracted with DCM. The combined organic phases were washed with brine, dried over Na₂SO₄, filtered and concentrated. The residue was purified by column chromatography to give tert-butyl (2S)-4,4-difluoro-2-methyl-pyrrolidine-1-carboxylate (3.2 g). ¹H NMR (400 MHz, CDCl₃) δ 4.24-3.98 (m, 1H), 3.90-3.56 (m, 2H), 2.63-2.42 (m, 1H), 2.13-1.89 (m, 1H), 1.47 (s, 9H), 1.30 (d, J=6.4 Hz, 3H).

4. Synthesis of compound (2S)-4,4-difluoro-2-methyl-pyrrolidine. To a solution of tert-butyl (2S)-4,4-difluoro-2-methyl-pyrrolidine-1-carboxylate (3.2 g, 14.5 mmol) in DCM (15 mL) was added TFA (5 mL, 67.3 mmol). The mixture was stirred at 25° C. for 16 h. The solvent was removed to yield (2S)-4,4-difluoro-2-methyl-pyrrolidine (3.4 g).

5. Synthesis of compound (2S)-4,4-difluoro-2-methyl-pyrrolidine-1-carbonitrile. To a solution of (2S)-4,4-difluoro-2-methyl-pyrrolidine (3.4 g, 14.5 mmol) in DCM (20 mL) were added sat. aq. NaHCO₃ (20 mL) dropwise at 0° C. and BrCN (1.99 g, 18.8 mmol). The mixture was stirred at 25° C. for 16 h. Water (100 mL) was added and the mixture was extracted with EA. The combined organic phases were washed with brine, dried over Na₂SO₄, filtered and concentrated. The residue was purified by column chromatography to give (2S)-4,4-difluoro-2-methyl-pyrrolidine-1-carbonitrile (2.0 g).

6. Synthesis of compound E41. Compound E41 was synthesized as compound E16. ¹H NMR (400 MHz, CDCl₃) δ 7.46-7.31 (m, 2H), 7.03-6.86 (m, 2H), 5.15-5.02 (m, 2H), 4.37 (q, J=4.8 Hz, 1H), 4.15 (ddd, J=3.2, 6.4, 9.2 Hz, 1H), 3.89-3.84 (m, 4H), 3.84-3.68 (m, 2H), 3.22-3.15 (m, 4H), 3.08 (d, J=4.8 Hz, 3H), 2.54-2.45 (m, 1H), 2.06 (dd, J=5.2, 8.8 Hz, 1H), 1.48 (s, 6H), 1.27 (d, J=6.4 Hz, 3H). LCMS (ESI): 530.3 [M+H]⁺.

### Example 119. Preparation of Compound E42

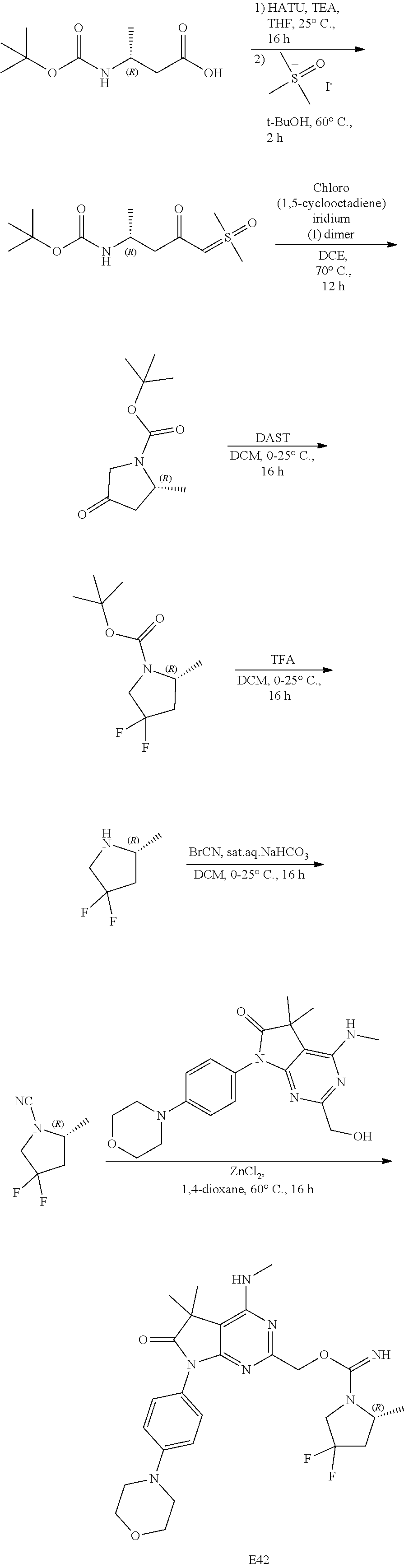

Compound E42 was synthesized as compound E41. ¹H NMR (400 MHz, CDCl₃) (δ 7.39-7.32 (m, 2H), 6.98-6.92 (m, 2H), 5.17-5.00 (m, 2H), 4.38 (q, J=4.8 Hz, 1H), 4.15 (d, J=6.0 Hz, 1H), 3.86 (t, J=4.8 Hz, 4H), 3.84-3.70 (m, 2H), 3.18 (t, J=4.8 Hz, 4H), 3.08 (d, J=4.8 Hz, 3H), 2.53-2.46 (m, 1H), 2.10-2.05 (m, 1H), 1.48 (s, 6H), 1.27 (d, J=6.8 Hz, 3H). LCMS (ESI): m/z, 530.3 [M+H]⁺.

### Example 120. Preparation of Compound E43

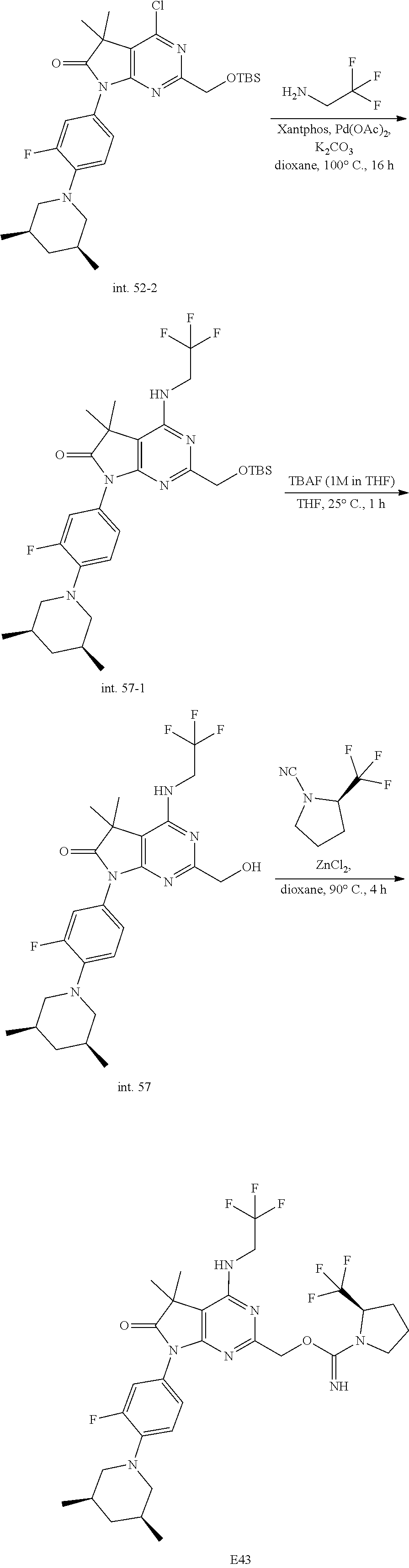 [SMILES: C[C@@H]1C[C@H](C)CN(c2ccc(N3C(=O)C(C)(C)c4c(Cl)nc(CO[Si](C)(C)C(C)(C)C)nc43)cc2F)C1 ; NCC(F)(F)F ; C[C@@H]1C[C@H](C)CN(c2ccc(N3C(=O)C(C)(C)c4c(NCC(F)(F)F)nc(CO[Si](C)(C)C(C)(C)C)nc43)cc2F)C1 ; C[C@@H]1C[C@H](C)CN(c2ccc(N3C(=O)C(C)(C)c4c(NCC(F)(F)F)nc(CO)nc43)cc2F)C1 ; N#CN1CCC[C@@H]1C(F)(F)F ; C[C@@H]1C[C@H](C)CN(c2ccc(N3C(=O)C(C)(C)c4c(NCC(F)(F)F)nc(COC(=N)N5CCC[C@@H]5C(F)(F)F)nc43)cc2F)C1]

Compound E43 was synthesized as compound E21. ¹H NMR (400 MHz, DMSO-d₆) δ 7.26-7.20 (m, 2H), 7.17-7.14 (m, 1H), 7.11-7.06 (m, 1H), 5.79 (s, 1H), 5.04-4.92 (m, 2H), 4.56-4.52 (m, 1H), 4.31-4.21 (m, 2H), 3.40-3.33 (m, 4H), 2.54-2.19 (m, 2H), 1.94-1.77 (m, 7H), 1.45 (s, 6H), 0.90 (s, 3H), 0.88 (s, 3H), 0.73-0.63 (m, 1H). LCMS (ESI): m/z, 660.5 [M+H]⁺.

### Example 121. Preparation of Compound E44

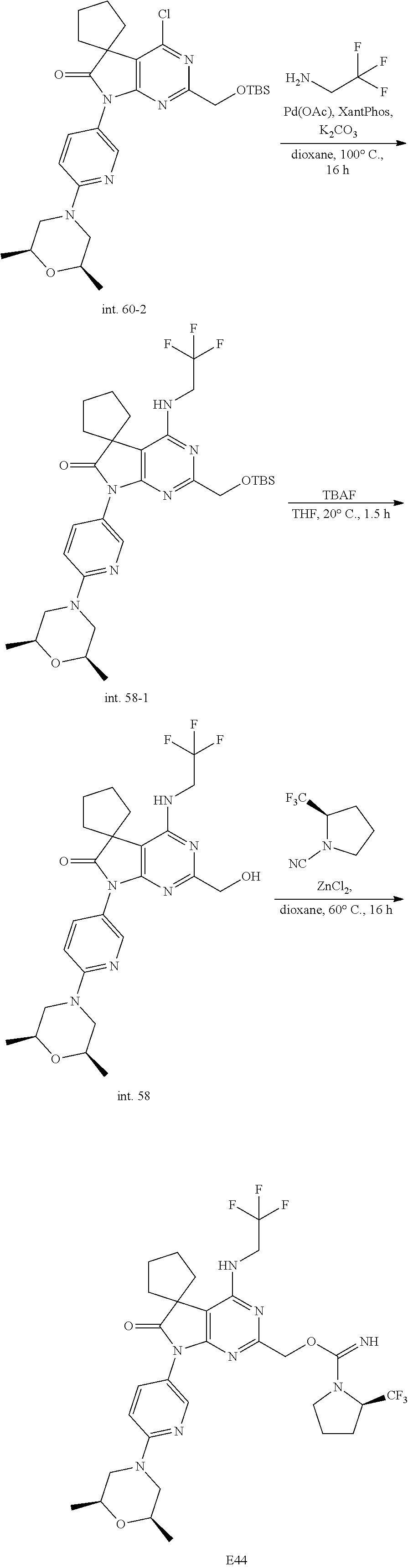

Compound E44 was synthesized as compound E21. ¹H NMR (400 MHz, Chloroform-d) δ 8.28 (s, 1H), 7.58 (d, J=9.2 Hz, 1H), 6.68 (d, J=9.2 Hz, 1H), 5.31-5.00 (m, 2H), 4.46 (q, J=7.6, 6.9 Hz, 2H), 4.31 (t, J=8.4 Hz, 2H), 4.09 (d, J=12.4 Hz, 2H), 3.71 (d, J=9.6 Hz, 2H), 3.46 (dd, J=8.8, 22.0 Hz, 2H), 2.56 (t, J=11.6 Hz, 2H), 2.25-1.87 (m, 12H), 1.28 (d, J=6.4 Hz, 6H). LCMS (ESI): m/z, 671.5[M+H]⁺.

### Example 122. Preparation of Compound E45

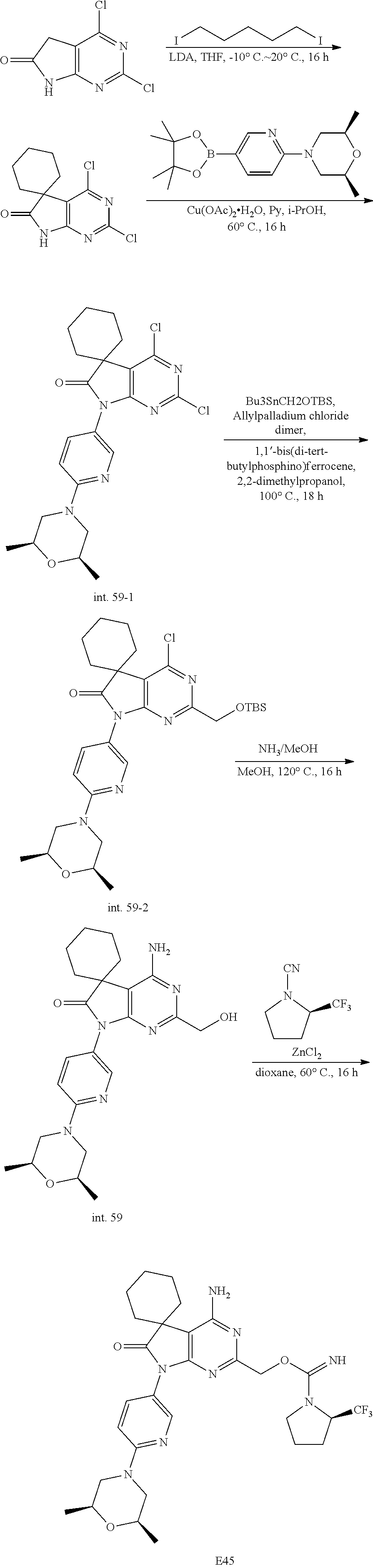

1. Synthesis of compound 2′,4′-dichlorospiro[cyclohexane-1,5′-pyrrolo[2,3-d]pyrimidin]-6′(7′H)-one. To a solution of 2,4-dichloro-5,7-dihydro-6H-pyrrolo[2,3-d]pyrimidin-6-one (3.0 g, 14.7 mmol) in THE (5 mL) was added LDA (4.0 g, 36.76 mmol, 2 M) at −20° C., the mixture was stirred at −20° C. for 1 h under N₂ atmosphere. Then 1,5-diiodopentane (10 g, 30.88 mmol) was added in the mixture at −20° C. for 16 h. The reaction was quenched by sat. aq. NH₄Cl (15 mL), and water (100 mL) was added and extracted with EtOAc. The combined organic phases were washed with brine, dried over Na₂SO₄, filtered and concentrated. The residue was purified by column chromatography to give 2′,4′-dichlorospiro[cyclohexane-1,5′-pyrrolo[2,3-d]pyrimidin]-6′(7′H)-one (1.77 g). ¹H NMR (400 MHz, DMSO-d₆) δ 11.96 (s, 1H), 2.01 (m, 4H), 1.84-1.66 (m, 4H), 1.62-1.54 (m, 2H). LCMS (ESI): m/z, 272.0 [M+H]⁺.

2. Synthesis of compound int. 59-1. Compound int. 59-1 was synthesized as compound int. 33-1. ¹H NMR (400 MHz, CDCl₃) δ 8.22 (d, J=2.4 Hz, 1H), 7.53-7.47 (m, 1H), 6.70 (d, J=9.2 Hz, 1H), 4.11 (dt, J=1.6, 12.8 Hz, 2H), 3.71 (m, 2H), 2.62-2.57 (m, 2H), 2.36-2.11 (m, 6H), 1.78-1.73 (m, 4H), 1.29-1.26 (m, 6H). LCMS (ESI): t_{R}=1.095, m/z, 462.2 [M+H]⁺.

3. Synthesis of compound int. 59-2. To a solution of 2′,4′-dichloro-7′-(6-((2S,6R)-2,6-dimethylmorpholino)pyridin-3-yl)spiro[cyclohexane-1,5′-pyrrolo[2,3-d]pyrimidin]-6′(7′H)-one (1.0 g, 2.2 mmol) and Bu₃SnCH₂OTBS (1.04 g, 2.4 mmol) in 2,2-dimethylpropanol (10 mL) were added Allylpalladium chloride dimer (40 mg, 0.11 mmol) and 1,1′-bis(di-tert-butylphosphino)ferrocene (103 g, 0.22 mmol) at N₂ atmosphere. The reaction was stirred at 100° C. for 18 h. The mixture was filtered and the filtrate was concentrated under reduce pressure to give a residue which was purified by column chromatography to give 2′-(((tert-butyldimethylsilyl)oxy)methyl)-4′-chloro-7′-(6-((2S,6R)-2,6-dimethylmorpholino)pyridin-3-yl)spiro[cyclohexane-1,5′-pyrrolo[2,3-d]pyrimidin]-6′(7′H)-one (590 mg crude). LCMS (ESI): m/z, 572.3 [M+H]⁺.

4. Synthesis of compound int. 59. To a solution of 2′-(((tert-butyldimethylsilyl)oxy)methyl)-4′-chloro-7′-(6-((2S,6R)-2,6-dimethylmorpholino)pyridin-3-y)spiro[cyclohexane-1,5′-pyrrolo[2,3-d]pyrimidin]-6′(7′H)-one (560 mg, 1.01 mmol) in MeOH (10 mL) was added NH₃ (20 mL, 7 M in MeOH). The mixture was stirred at 120° C. for 64 h under sealing tube. The solvent was removed to yield a residue which was purified by column chromatography to give a 4′-amino-7′-(6-((2S,6R)-2,6-dimethylmorpholino)pyridin-3-yl)-2′-(hydroxymethyl)spiro[cyclohexane-1,5′-pyrrolo[2,3-d]pyrimidin]-6′(7′H)-one (370 mg crude). ¹H NMR (400 MHz, CDCl₃) δ 8.25 (d, J=2.4 Hz, 1H), 7.55 (dd, J=2.8, 9.2 Hz, 1H), 6.70 (d, J=9.2 Hz, 1H), 4.88 (s, 2H), 4.51 (s, 2H), 4.09 (d, J=12.4 Hz, 2H), 3.73 (m, 2H), 3.52 (s, 1H), 2.58 (t, J=10.4, 12.8 Hz, 2H), 2.29-2.16 (m, 2H), 1.98-1.88 (m, 5H), 1.28 (d, J=6.4 Hz, 6H). LCMS (ESI): m/z, 521.2 [M+H]⁺.

5. Synthesis of compound E45. Compound E45 was synthesized as compound E16. ¹H NMR (400 MHz, CDCl₃) δ 8.26 (d, J=2.4 Hz, ¹H), 7.55 (dd, J=2.8, 9.2 Hz, 1H), 6.70 (d, J=9.2 Hz, ¹H), 4.89 (s, 2H), 4.52 (s, 2H), 4.10 (d, J=12.4 Hz, 2H), 3.73 (m, 2H), 3.52 (s, 1H), 2.58 (dd, J=10.4, 12.8 Hz, 2H), 2.28-2.14 (m, 2H), 1.99-1.85 (m, 5H), 1.28 (d, J=6.4 Hz, 6H). LCMS (ESI): m/z, 603.5 [M+H]⁺.

### Example 123. Preparation of Compound E46

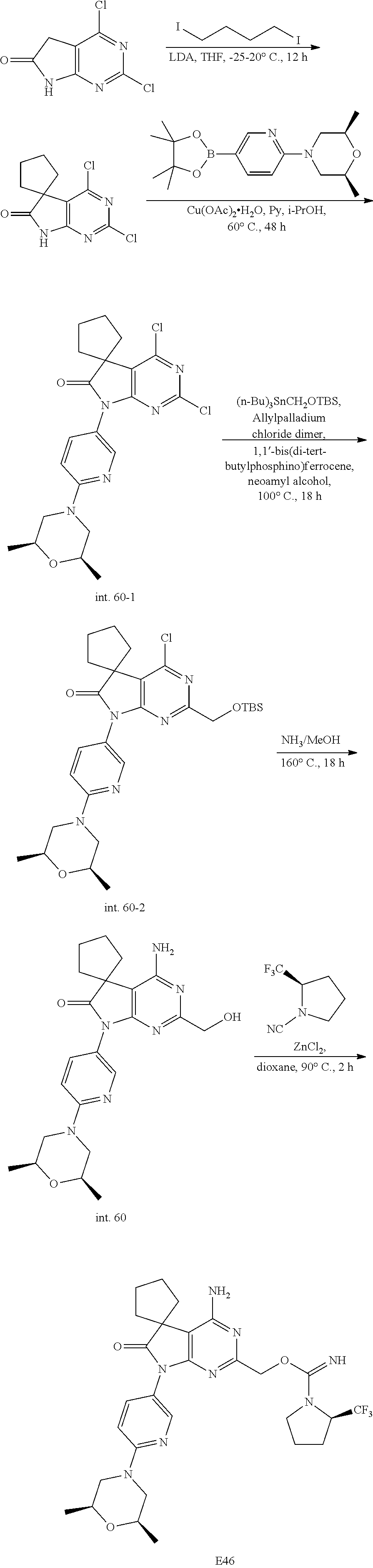

Compound E46 was synthesized as compound E45. ¹H NMR (400 MHz, Chloroform-d) b 8.29 (d, J=2.6 Hz, 1H), 7.60 (dd, J=2.8, 9.2 Hz, 1H), 6.67 (d, J=9.2 Hz, 1H), 5.26-4.94 (m, 2H), 4.82 (s, 2H), 4.45 (t, J=7.8 Hz, 1H), 4.08 (d, J=12.4 Hz, 2H), 3.81-3.60 (m, 2H), 3.43 (dd, J=8.8, 18.0, 8 Hz, 2H), 2.55 (t, J=11.6 Hz, 2H), 2.17-2.08 (m, 8H), 1.98-1.85 (m, 4H), 1.28 (d, J=6.0 Hz, 6H). LCMS (ESI): m/z, 589.4[M+H]⁺.

### Example 124. Preparation of Compound E47

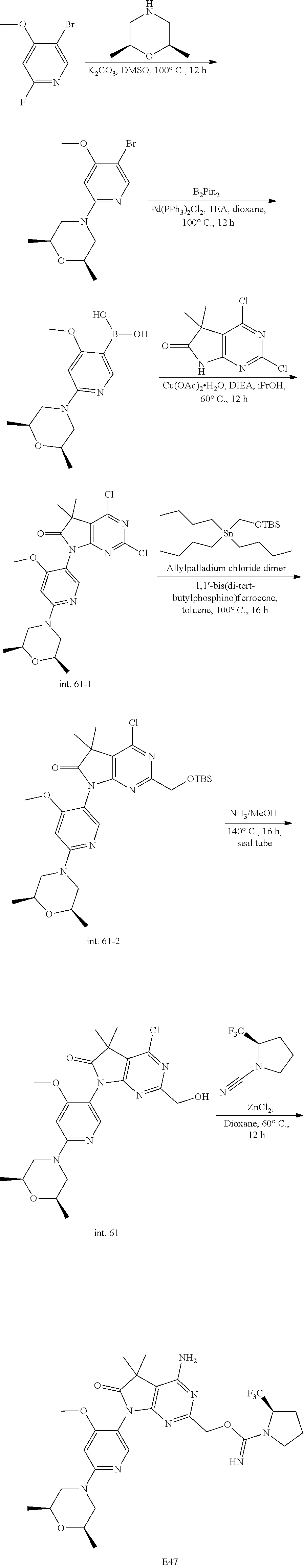

Compound E47 was synthesized as compound E34. ¹H NMR (400 MHz, CDCl₃) δ 7.78 (d, J=2.8 Hz, 1H), 6.74 (s, 2H), 6.46 (d, J=2.0 Hz, 1H), 5.81 (brs, 1H), 4.93-4.78 (s, 2H), 4.48-4.44 (m, 1H), 4.26-4.23 (m, 2H), 3.73 (s, 3H), 3.63-3.56 (m, 2H), 3.25-3.17 (m, 2H), 2.45-2.39 (m, 2H), 1.86-1.78 (m, 4H), 1.38 (d, J=13.2 Hz, 6H), 1.17 (d, J=6.4 Hz, 6H). LCMS (ESI): t_{R}=1.726, m/z, 593.3 [M+H]⁺.

### Example 125. Preparation of Compound E48

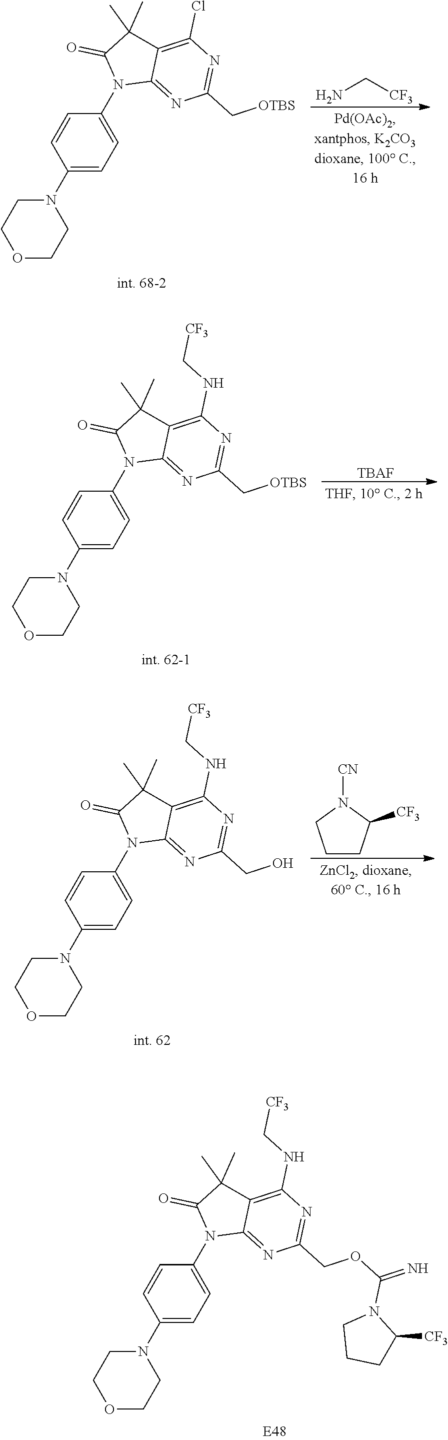

Compound E48 was synthesized as compound E21. ¹H NMR (400 MHz, CDCl₃) δ 7.39-7.30 (m, 2H), 6.96 (d, J=9.2 Hz, 2H), 5.28-5.03 (m, 2H), 4.53 (t, J=6.4 Hz, 1H), 4.45 (t, J=7.6 Hz, 1H), 4.33-4.28 (m, 2H), 3.94-3.82 (m, 4H), 3.49-3.39 (m, 2H), 3.27-3.11 (m, 4H), 2.10-2.06 (m, 2H), 2.00-1.85 (m, 2H), 1.51 (s, 6H). LCMS (ESI): m/z, 616.4 [M+H]⁺.

### Example 126. Preparation of Compound E49

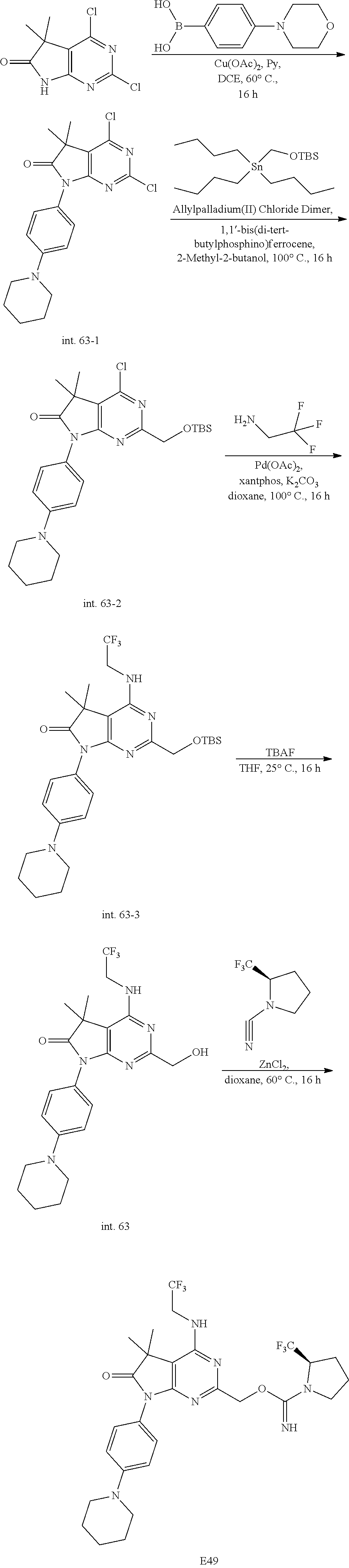

Compound E49 was synthesized as compound E50. ¹H NMR (400 MHz, CDCl₃) δ 7.28 (d, J=9.2 Hz, 2H), 6.96 (d, J=9.2 Hz, 2H), 5.29 (d, J=15.2 Hz, ¹H), 5.09 (d, J=15.2 Hz, 1H), 4.50 (dt, J=7.2, 27.6 Hz, 2H), 4.30 (dd, J=6.4, 9.2 Hz, 2H), 3.52-3.39 (m, 2H), 3.22-3.16 (m, 4H), 2.06 (d, J=9.6 Hz, 2H), 1.93 (d, J 8.0 Hz, 2H), 1.82 (d, J 8.4 Hz, 2H), 1.73 (s, 1H), 1.60 (d, J 5.2 Hz, 3H), 1.51 (s, 6H). LCMS (ESI): m/z, 614.4 [M+H]⁺.

### Example 127. Preparation of Compound E50

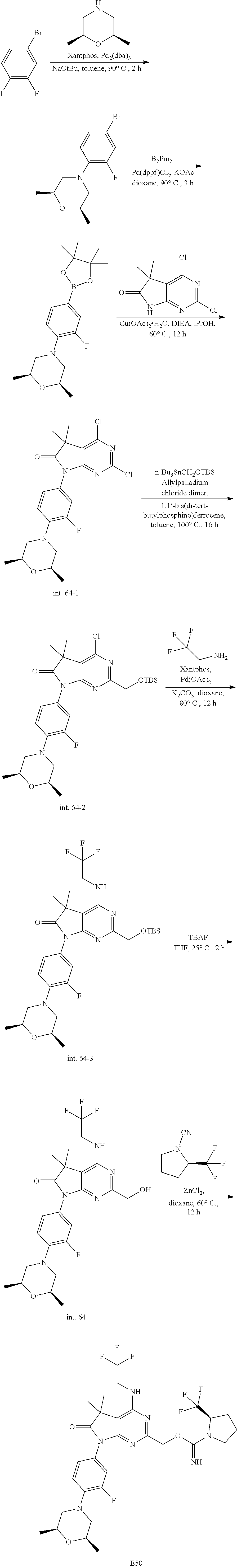

1. Synthesis of compound (2S,6R)-4-(4-bromo-2-fluorophenyl)-2,6-dimethylmorpholine. A mixture of 1-bromo-3-fluoro-4-iodobenzene (31.3 g, 104 mmol), cis-2,6-dimethylmorpholine (10.75 mL), XantPhos (5.02 g, 8.68 mmol), Pd₂(dba)₃ (3.98 g, 4.34 mmol), and t-BuONa (20.9 g, 217 mmol) in PhMe (200 mL) was stirred at 90° C. for 12 h under N₂ atmosphere. The mixture was concentrated. The residue was purified by column chromatography to afford (2S,6R)-4-(4-bromo-2-fluorophenyl)-2,6-dimethylmorpholine (19.0 g). LCMS (ESI): m/z, 288.1, 290.1 [M+H]⁺.

2. Synthesis of compound (2S,6R)-4-(2-fluoro-4-(4,4,5,5-tetramethyl-1,3,2-dioxaborolan-2-yl)phenyl)-2,6-dimethylmorpholine. A mixture of (2R,6S)-4-(4-bromo-2-fluorophenyl)-2,6-dimethylmorpholine (19.0 g, 65.9 mmol), Pin₂B₂ (25.1 g, 98.9 mmol), and Pd(dppf)Cl₂ (3.84 g, 5.27 mmol), AcOK (12.9 g, 132 mmol) in 1,4-dioxane (500 mL) was stirred at 90° C. for 3 h under N₂ atmosphere. The mixture was concentrated. The residue was purified by column chromatography to afford (2S,6R)-4-(2-fluoro-4-(4,4,5,5-tetramethyl-1,3,2-dioxaborolan-2-yl)phenyl)-2,6-dimethylmorpholine (20.0 g). LCMS (ESI): m/z, 336.2 [M+H]⁺. [00846]3. Synthesis of compound int. 64-1. A mixture of 2,4-dichloro-5,5-dimethyl-5H,6H,7H-pyrrolo[2,3-d]pyrimidin-6-one (3.0 g, 12.9 mmol), (2S,6R)-4-(2-fluoro-4-(4,4,5,5-tetramethyl-1,3,2-dioxaborolan-2-yl)phenyl)-2,6-dimethylmorpholine (6.50 g, 19.4 mmol), Cu(OAc)₂·H₂O (2.58 g, 12.9 mmol), DIPEA (4.27 mL), and 4 Å MS (9.11 g) in i-PrOH (100 mL) was stirred at 60° C. for 12 h under 02 atmosphere. The mixture was filtered and the filtrate was concentrated. The residue was purified by column chromatography to afford 2,4-dichloro-7-(4-((2S,6R)-2,6-dimethylmorpholino)-3-fluorophenyl)-5,5-dimethyl-5,7-dihydro-6H-pyrrolo[2,3-d]pyrimidin-6-one (2.55 g). LCMS (ESI): m/z, 439.1, 441.1 [M+H]⁺.

4. Synthesis of compound int. 64-2. A mixture of 2,4-dichloro-7-(4-((2S,6R)-2,6-dimethylmorpholino)-3-fluorophenyl)-5,5-dimethyl-5,7-dihydro-6H-pyrrolo[2,3-d]pyrimidin-6-one (2.55 g, 5.80 mmol), Bu₃SnCH₂OTBS (2.78 g, 6.39 mmol), 1,1′-bis(di-tert-butylphosphino)ferrocene (275 mg, 0.580 mmol), and allylpalladium chloride dimer (106 mg, 0.290 mmol) in PhMe (20 mL) was stirred at 100° C. for 12 h under N₂ atmosphere. The mixture was concentrated. The residue was purified by column chromatography to afford 2-(((tert-butyldimethylsilyl)oxy)methyl)-4-chloro-7-(4-((2S,6R)-2,6-dimethylmorpholino)-3-fluorophenyl)-5,5-dimethyl-5,7-dihydro-6H-pyrrolo[2,3-d]pyrimidin-6-one (1.80 g). LCMS (ESI): m/z, 549.3 [M+H]⁺.

5. Synthesis of compound int. 64-3. A mixture of 2-(((tert-butyldimethylsilyl)oxy)methyl)-4-chloro-7-(4-((2S,6R)-2,6-dimethylmorpholino)-3-fluorophenyl)-5,5-dimethyl-5,7-dihydro-6H-pyrrolo[2,3-d]pyrimidin-6-one (600 mg, 1.09 mmol), 2,2,2-trifluoroethylamine (541 mg, 5.46 mmol), XantPhos (63 mg, 0.109 mmol), Pd(OAc)₂ (18 mg, 0.109 mmol), and K₂CO₃ (302 mg, 2.19 mmol) in 1,4-dioxane (10 mL) was stirred at 80° C. for 12 h under N₂ atmosphere. The mixture was filtered, and the filtrate was concentrated to afford 2-(((tert-butyldimethylsilyl)oxy)methyl)-7-(4-((2S,6R)-2,6-dimethylmorpholino)-3-fluorophenyl)-5,5-dimethyl-4-((2,2,2-trifluoroethyl)amino)-5,7-dihydro-6H-pyrrolo[2,3-d]pyrimidin-6-one (600 mg crude). LCMS (ESI): m/z, 612.2 [M+H]⁺.

6. Synthesis of compound int. 64. To a solution of 2-(((tert-butyldimethylsilyl)oxy)methyl)-7-(4-((2S,6R)-2,6-dimethylmorpholino)-3-fluorophenyl)-5,5-dimethyl-4-((2,2,2-trifluoroethyl)amino)-5,7-dihydro-6H-pyrrolo[2,3-d]pyrimidin-6-one (600 mg, 0.980 mmol) in THE (25 mL) was added TBAF (2.94 mL, 1 M). The mixture was stirred at 25° C. for 2 h. The mixture was concentrated. The residue was purified by column chromatography to afford 7-(4-((2S,6R)-2,6-dimethylmorpholino)-3-fluorophenyl)-2-(hydroxymethyl)-5,5-dimethyl-4-((2,2,2-trifluoroethyl)amino)-5,7-dihydro-6H-pyrrolo[2,3-d]pyrimidin-6-one (350 mg). LCMS (ESI): m/z, 598.4 [M+H]⁺.

7. Synthesis of compound E50. Compound E50 was synthesized as compound E16. ¹H NMR (400 MHz, CDCl₃) δ 7.29-7.28 (m, 2H), 7.01-6.97 (m, 1H), 5.26-5.11 (m, 2H), 4.62 (t, J=6.8 Hz, 1H), 4.49-4.52 (m, 1H), 4.38-4.28 (m, 2H), 3.94-3.87 (m, 2H), 3.54-3.41 (m, 2H), 3.34-3.29 (m, 2H), 2.53-2.46 (m, 2H), 2.17-2.09 (m, 2H), 2.04-1.92 (m, 2H), 1.54 (s, 6H), 1.26 (d, J=6.4 Hz, 6H). LCMS (ESI): m/z, 662.5 [M+H]⁺.

### Example 128. Preparation of Compound E51

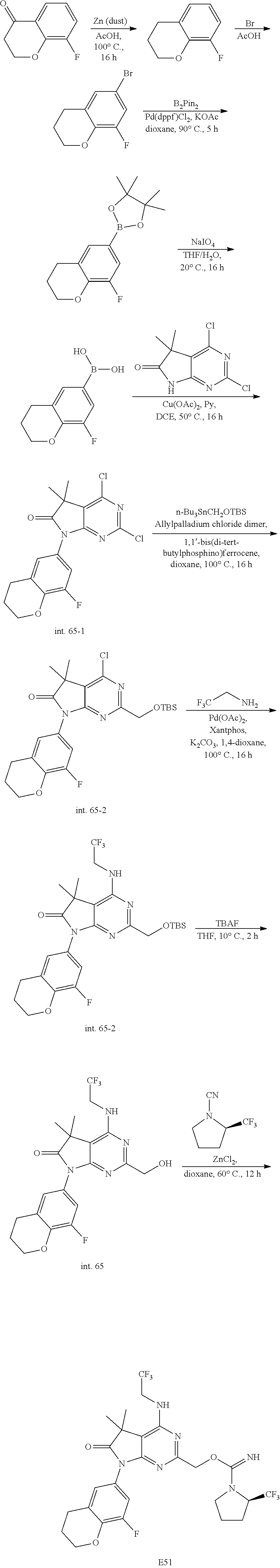

1. Synthesis of compound 8-fluoro-3,4-dihydro-2H-1-benzopyran. To a solution of 8-fluoro-2,3-dihydro-4-H-1-benzopyran-4-one (1.40 g, 8.43 mmol) in AcOH (20 mL) was added Zn (2.75 g, 42.1 mmol). The mixture was stirred at 100° C. for 16 h. The mixture was filtered and the filtrate was concentrated. The residue was diluted with water, adjusted pH to 8 with sat. aq. NaHCO₃, then extracted with EtOAc. The combined organic phases were washed with brine, dried over anhydrous Na₂SO₄, filtered and concentrated. The crude was purified by column chromatography to give 8-fluoro-3,4-dihydro-2H-1-benzopyran (600 mg). ¹H NMR (400 MHz, CDCl₃) δ 6.94-6.82 (m, 1H), 6.84-6.70 (m, 2H), 4.32-4.21 (m, 2H), 2.81 (t, J=6.4 Hz, 2H), 2.10-1.97 (m, 2H).

2. Synthesis of compound 6-bromo-8-fluoro-3,4-dihydro-2H-l-benzopyran. To a solution of 8-fluoro-3,4-dihydro-2H-1-benzopyran (600 mg, 3.94 mmol) in AcOH (10 mL) was added a solution of Br₂ (0.24 mL) in AcOH (1 mL) dropwise at 20° C. and stirred for 2 h. Water was added and the mixture was extracted with EtOAc. The combined organic phases were washed with sat. aq. NaHCO₃ (20 mL) and brine (20 mL), dried over anhydrous Na₂SO₄, filtered and concentrated. The crude product was purified by column chromatography to give 6-bromo-8-fluoro-3,4-dihydro-2H-l-benzopyran (836 mg).

3. Synthesis of compound 2-(8-fluoro-3,4-dihydro-2H-1-benzopyran-6-yl)-4,4,5,5-tetramethyl-1,3,2-dioxaborolane. To a solution of 6-bromo-8-fluoro-3,4-dihydro-2H-1-benzopyran (836 mg, 3.62 mmol) and Pin₂B₂ (1.19 g, 4.70 mmol) in 1,4-dioxane (10 mL) were added KOAc (710.16 mg, 7.24 mmol) and Pd(dppf)Cl₂ (265 mg, 0.36 mmol). The mixture was stirred at 90° C. for 5 h under N₂ atmosphere. The solvent was removed and the residue was treated with EtOAc. The mixture was filtered and the filtrate was concentrated. The crude was purified by column chromatography to give 2-(8-fluoro-3,4-dihydro-2H-1-benzopyran-6-yl)-4,4,5,5-tetramethyl-1,3,2-dioxaborolane (1.00 g). LCMS (ESI): m/z, 279.2 [M+H]⁺.

4. Synthesis of compound (8-fluoro-3,4-dihydro-2H-1-benzopyran-6-yl)boronic acid. To a solution of 2-(8-fluoro-2H-chromen-6-yl)-4,4,5,5-tetramethyl-1,3,2-dioxaborolane (700 mg, 2.54 mmol) in THF (12 mL) and H₂O (4 mL) was added sodium periodate (1.64 g, 7.61 mmol). The mixture was stirred at 20° C. for 16 h. The mixture was filtered and the filtrate was concentrated. The crude was purified by column chromatography to give (8-fluoro-3,4-dihydro-2H-1-benzopyran-6-yl)boronic acid (400 mg).

5. Synthesis of compound E51. Other steps of compound E51 were synthesized as compound E50. ¹H NMR (400 MHz, CDCl₃) δ 7.06 (dd, J=2.4, 11.2, Hz, 1H), 6.96 (dd, J=1.6, 2.4, 1H), 5.28-5.05 (m, 2H), 4.54 (t, J=6.4 Hz, 1H), 4.44 (t, J=7.6 Hz, 1H), 4.37-4.25 (m, 4H), 3.55-3.35 (m, 2H), 2.84 (t, J=6.4 Hz, 2H), 2.13-1.91 (m, 6H), 1.51 (s, 6H). LCMS (ESI): m/z, 605.4 [M+H]⁺.

### Example 129. Preparation of Compound E52

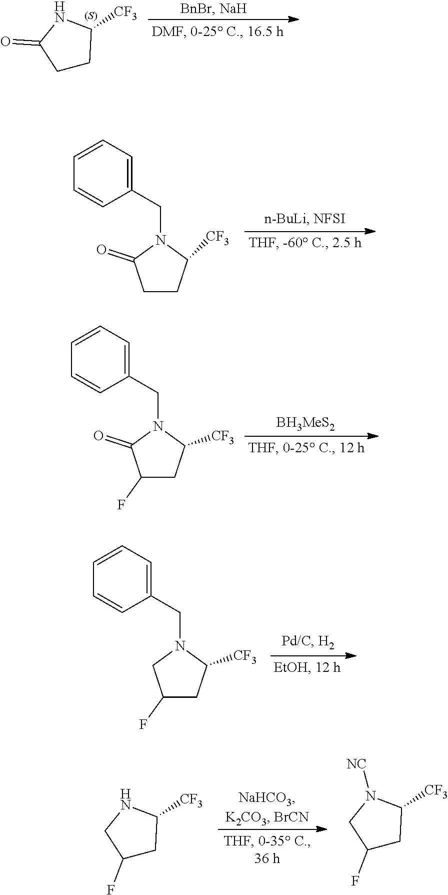

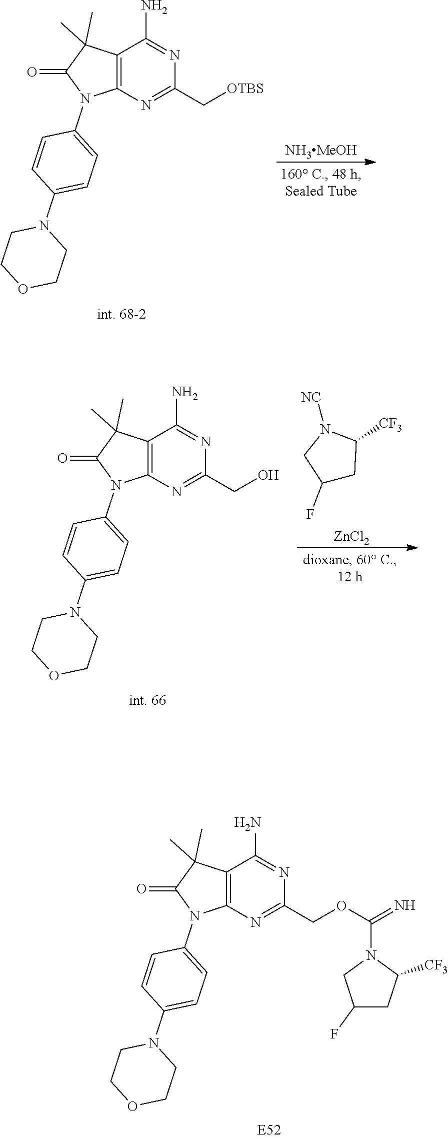

1. Synthesis of compound (S)-1-benzyl-5-(trifluoromethyl)pyrrolidin-2-one. To a solution of (S)-5-(trifluoromethyl)pyrrolidin-2-one (750 mg, 4.90 mmol) in DMF (3 mL) was added NaH (235 mg, 60%, 5.88 mmol) at 0° C., the mixture was stirred at 25° C. for 0.5 hour under N₂ atmosphere, then a solution of (bromomethyl)benzene (922 mg, 5.39 mmol) in DMF (1 mL) was dropwise added to the mixture at 25° C., and the mixture was stirred at 25° C. for 16 h under N₂ atmosphere. The mixture was quenched with sat. aq. NH₄Cl (20 mL), then extracted with EtOAc, and the organic layer was washed with brine, dried over Na₂SO₄, filtered and concentrated. The residue was purified by column chromatography to give compound (S)-1-benzyl-5-(trifluoromethyl)pyrrolidin-2-one (1.10 g). LCMS (ESI): m/z, 244.1 [M+H]⁺.

2. Synthesis of compound (5S)-1-benzyl-3-fluoro-5-(trifluoromethyl)pyrrolidin-2-one. To a solution of (S)-1-benzyl-5-(trifluoromethyl)pyrrolidin-2-one (1.1 g, 4.52 mmol) in THF (30 mL) was added n-BuLi (6.78 mmol, 2.5 M in hexane) dropwise at −60° C. and stirred at −60° C. for 30 min. A solution of NFSI (2.85 g, 9.04 mmol) in THF (5 mL) was added dropwise at −60° C. The mixture was stirred at −60° C. for 2 h, then was quenched by sat. aq. NH₄Cl (10 mL), and water (20 mL) was added and extracted with EtOAc. The combined organic phases were washed with brine, dried over anhydrous Na₂SO₄, filtered and concentrated. The residue was purified by column chromatography to give compound (5S)-1-benzyl-3-fluoro-5-(trifluoromethyl)pyrrolidin-2-one (568 mg). LCMS (ESI): m/z, 262.1 [M+H]⁺.

3. Synthesis of compound (2S)-1-benzyl-4-fluoro-2-(trifluoromethyl)pyrrolidine. To a solution of (5S)-1-benzyl-3-fluoro-5-(trifluoromethyl)pyrrolidin-2-one (50 mg, 0.19 mmol) in THF (1 mL) was dropwise added BMS (0.57 mmol, 2 M in THF) at 0° C., and the mixture was stirred at 25° C. for 12 h. The mixture was quenched with MeOH (2 mL) and concentrated. Then aq. HCl (2M, 3 mL) was dropwise added to the mixture and stirred for 15 mins, and the mixture was concentrated to give compound (2S)-1-benzyl-4-fluoro-2-(trifluoromethyl)pyrrolidine (49 mg crude). LCMS (ESI): m/z, 248.1 [M+H]⁺.

4. Synthesis of compound (2S)-4-fluoro-2-(trifluoromethyl)pyrrolidine. To a solution of (2S)-1-benzyl-4-fluoro-2-(trifluoromethyl)pyrrolidine (620 mg, 2.51 mmol) in EtOH (15 mL) was added Pd/C (300 mg, 10%), and the mixture was stirred at 25° C. for 12 h under H₂ (15 psi) atmosphere. The mixture was filtered, aq. HCl (2 M, 3 mL) and water (20 mL) was added to the mixture and stirred for 10 min, then washed with DCM. The water phase was lyophilization to give compound (2S)-4-fluoro-2-(trifluoromethyl)pyrrolidine (740 mg crude). ¹H NMR (400 MHz, DMSO) δ 5.51 (d, J=3.6 Hz, 1H), 4.81-4.66 (m, 1H), 3.65-3.45 (m, 2H), 2.69-2.54 (m, 1H), 2.39-2.22 (m, 1H). LCMS (ESI): m/z, 158.0 [M+H]⁺.

5. Synthesis of compound (2S)-4-fluoro-2-(trifluoromethyl)pyrrolidine-1-carbonitrile. To a solution of (2S)-4-fluoro-2-(trifluoromethyl)pyrrolidine (640 mg, 3.31 mmol) in THF (5 mL) was added sat. aq. NaHCO₃ (18.4 g, 10 mL) at 0° C., then a solution of BrCN (700 mg, 6.61 mmol) in THE (1 mL) was dropwise added to the mixture at 0° C., and the mixture was stirred at 35° C. for 18 h. K₂CO₃ (274 mg, 1.98 mmol) and BrCN (350 mg, 3.31 mmol) were added to the mixture and stirred at 25° C. for 18 h. The mixture was concentrated to remove THF, the water phase was extracted with PE/EA=5:1, and the organic layer was washed with water, dried over Na₂SO₄, filtered and concentrated. The residue was purified by column chromatography to give compound (2S)-4-fluoro-2-(trifluoromethyl)pyrrolidine-1-carbonitrile (106 mg). ¹H NMR (400 MHz, CDCl₃) δ 5.39-5.20 (m, 1H), 4.34 (dd, J=14.4, 8.0 Hz, 1H), 3.87-3.61 (m, 2H), 2.67-2.53 (m, 1H), 2.26-2.10 (m, 1H).

6. Synthesis of compound int. 66. Compound int. 66 was synthesized as compound int. 38. LCMS (ESI): m/z, 370.1 [M+H]⁺.

7. Synthesis of compound E52. Compound E52 was synthesized as compound E20. ¹H NMR (400 MHz, CDCl3) δ 7.36 (d, J=9.0 Hz, 2H), 6.97 (d, J=9.0 Hz, 2H), 5.28 (d, J=53.8 Hz, 1H), 5.07 (d, J=7.0 Hz, 2H), 4.79 (s, 3H), 4.18 (dd, J=21.6, 12.8 Hz, 1H), 3.91-3.82 (m, 4H), 3.59-3.56 (m, 1H), 3.24-3.16 (m, 4H), 2.45-2.26 (m, 2H), 1.51 (s, 6H). LCMS (ESI): m/z, 552.5 [M+H]⁺.

### Example 130. Preparation of Compound E53

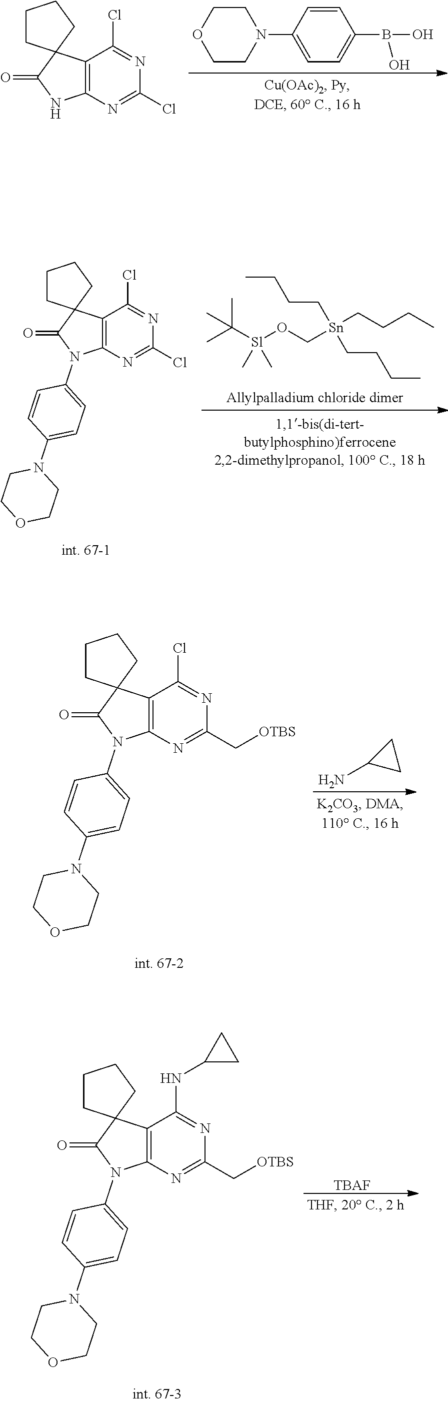

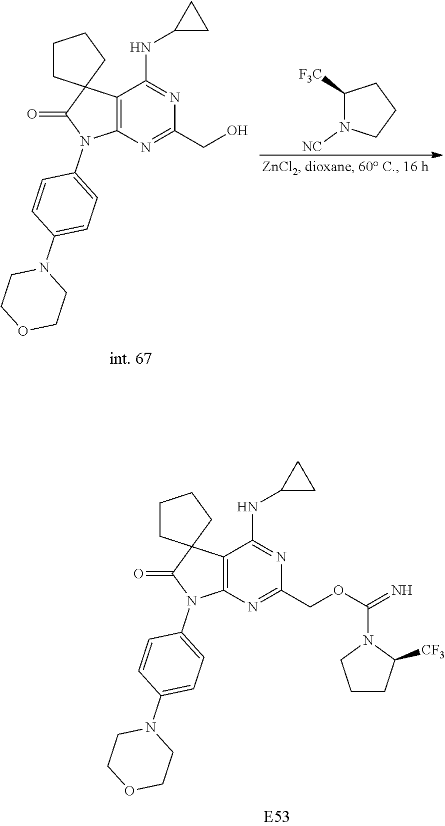 [SMILES: O=C1N(c2ccc(N3CCOCC3)cc2)c2nc(CO)nc(NC3CC3)c2C12CCCC2 ; N#CN1CCC[C@@H]1C(F)(F)F ; N=C(OCc1nc(NC2CC2)c2c(n1)N(c1ccc(N3CCOCC3)cc1)C(=O)C21CCCC1)N1CCC[C@@H]1C(F)(F)F]

1. Synthesis of compound int. 67-1. To a solution of 2′,4′-dichlorospiro[cyclopentane-1,5′-pyrrolo[2,3-d]pyrimidin]-6′(7′H)-one (5.50 g, 21.3 mmol) in 1,2-dichloroethane (100 mL) were added (4-morpholinophenyl)boronic acid (6.62 g, 32.0 mmol), pyridine (5.17 mL), Cu(OAc)₂ (4.64 g, 25.6 mmol) and 4A Molecular Sieve (5.50 g). The mixture was stirred at 60° C. for 16 h with O₂ atmosphere. The mixture was filtered and concentrated. The residue was purified by column chromatography to give 2′,4′-dichloro-7′-(4-morpholinophenyl)spiro[cyclopentane-1,5′-pyrrolo[2,3-d]pyrimidin]-6′(7′H)-one (3.50 g). LCMS (ESI): m/z, 419.2, 421.2 [M+H]⁺.

2. Synthesis of compound int. 67-2. To a solution of 2′,4′-dichloro-7′-(4-morpholinophenyl)spiro[cyclopentane-1,5′-pyrrolo[2,3-d]pyrimidin]-6′(7′H)-one (3.50 g, 8.35 mmol) in 2,2-dimethylpropanol (50 mL) were added allylpalladium chloride dimer (153 mg, 0.42 mmol), 1,1′-bis(di-tert-butylphosphino)ferrocene (396 mg, 0.83 mmol) and tert-butyldimethyl((tributylstannyl)methoxy)silane (4.0 g, 9.18 mmol), the mixture was stirred at 100° C. for 18 h with N₂ atmosphere. The mixture was filtered and concentrated. The residue was purified by column chromatography to give 2′-(((tert-butyldimethylsilyl)oxy)methyl)-4′-chloro-7′-(4-morpholinophenyl)spiro[cyclopentane-1,5′-pyrrolo[2,3-d]pyrimidin]-6′(7′H)-one (1.95 g). LCMS (ESI): 529.3, 531.3 [M+H]⁺.

3. Synthesis of compound int. 67-3. To a solution of 2′-(((tert-butyldimethylsilyl)oxy)methyl)-4′-chloro-7′-(4-morpholinophenyl)spiro[cyclopentane-1,5′-pyrrolo[2,3-d]pyrimidin]-6′(7′H)-one (1.20 g, 2.27 mmol) in DMA (20 mL) were added K₂CO₃ (940 mg, 6.80 mmol) and cyclopropylamine (1.29 g, 22.67 mmol), the mixture was stirred at 110° C. for 16 h. To the mixture was added H₂O and EtOAc, the organic phase was separated and washed with H₂O. The combined organic layers were washed with brine and dried over Na₂SO₄, filtered and concentrated to give 2′-(((tert-butyldimethylsilyl)oxy)methyl)-4′-(cyclopropylamino)-7′-(4-morpholinophenyl)spiro[cyclopentane-1,5′-pyrrolo[2,3-d]pyrimidin]-6′(7′H)-one (1.20 g crude). LCMS (ESI): m/z, 550.4 [M+H]⁺.

4. Synthesis of compound int. 67. To a solution of 2′-(((tert-butyldimethylsilyl)oxy)methyl)-4′-(cyclopropylamino)-7′-(4-morpholinophenyl)spiro[cyclopentane-1,5′-pyrrolo[2,3-d]pyrimidin]-6′(7′H)-one (1.0 g, 1.82 mmol) in THF (10 mL) was add TBAF (5.46 mL 1 M in THF), the mixture was stirred at 20° C. for 2 h. To the mixture was added H₂O and extracted with EtOAc. The combined organic layers were washed with brine and dried over Na₂SO₄, filtered and concentrated. The residue was purified by column chromatography to give 4′-(cyclopropylamino)-2′-(hydroxymethyl)-7′-(4-morpholinophenyl)spiro[cyclopentane-1,5′-pyrrolo[2,3-d]pyrimidin]-6′(7′H)-one (700 mg). LCMS (ESI): m/z, 436.3 [M+H]⁺.

5. Synthesis of compound E53. To a solution of 4′-(cyclopropylamino)-2′-(hydroxymethyl)-7′-(4-morpholinophenyl)spiro[cyclopentane-1,5′-pyrrolo[2,3-d]pyrimidin]-6′(7′H)-one (650 mg, 1.49 mmol) in 1,4-dioxane (10 mL) were added (R)-2-(trifluoromethyl)pyrrolidine-1-carbonitrile (735 mg, 4.48 mmol) and ZnCl₂ (814 mg, 5.97 mmol). The mixture was stirred at 60° C. for 16 h under N₂ atmosphere. The residue was purified by prep-HPLC to give (4′-(cyclopropylamino)-7′-(4-morpholinophenyl)-6′-oxo-6′,7′-dihydrospiro[cyclopentane-1,5′-pyrrolo[2,3-d]pyrimidin]-2′-yl)methyl (R)-2-(trifluoromethyl)pyrrolidine-1-carbimidate (320 mg). ¹H NMR (400 MHz, DMSO-d₆) δ 7.20 (d, J=8.8 Hz, 2H), 7.00 (d, J=8.8 Hz, 2H), 6.33 (d, J=2.8 Hz, 1H), 5.10-4.88 (m, 2H), 4.57 (p, J=8.0 Hz, 1H), 3.75 (t, J=4.8 Hz, 4H), 3.33 (s, 2H), 3.14 (t, J=4.8 Hz, 4H), 2.83 (tq, J=3.6, 7.2 Hz, 1H), 2.23-2.08 (m, 2H), 2.03-1.73 (m, 10H), 0.79-0.70 (m, 2H), 0.58-0.56 (m, 2H). LCMS (ESI): m/z, 600.3[M+H]⁺.

### Example 131. Preparation of Compound E54

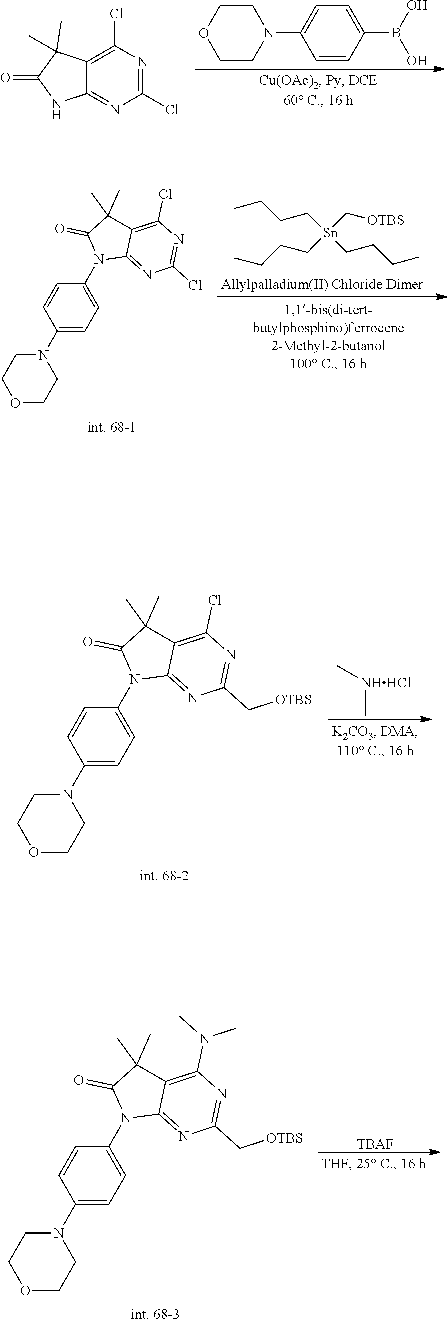

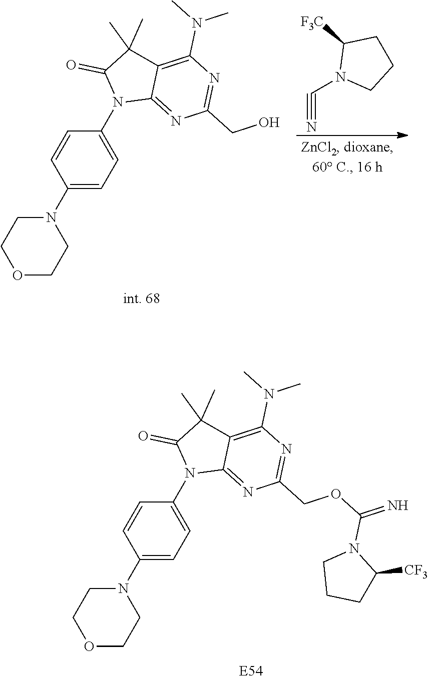 [SMILES: CN(C)c1nc(CO)nc2c1C(C)(C)C(=O)N2c1ccc(N2CCOCC2)cc1 ; N#CN1CCC[C@@H]1C(F)(F)F ; CN(C)c1nc(COC(=N)N2CCC[C@@H]2C(F)(F)F)nc2c1C(C)(C)C(=O)N2c1ccc(N2CCOCC2)cc1]

Compound E54 was synthesized as compound E53. ¹H NMR (400 MHz, Chloroform-d) b 7.32 (d, J=8.8 Hz, 2H), 6.95 (d, J=8.8 Hz, 2H), 5.17 (d, J=14.4 Hz, 1H), 4.98 (d, J=14.4 Hz, 1H), 4.52-4.41 (m, 1H), 3.90-3.83 (m, 4H), 3.52-3.38 (m, 2H), 3.22 (s, 6H), 3.19 (dd, J=3.6, 6.0 Hz, 4H), 2.07 (d, J=8.8 Hz, 2H), 1.85 (s, 2H), 1.59 (s, 6H). LCMS (ESI): m/z, 562.4 [M+H]⁺.

### Example 132. Preparation of Compound E55

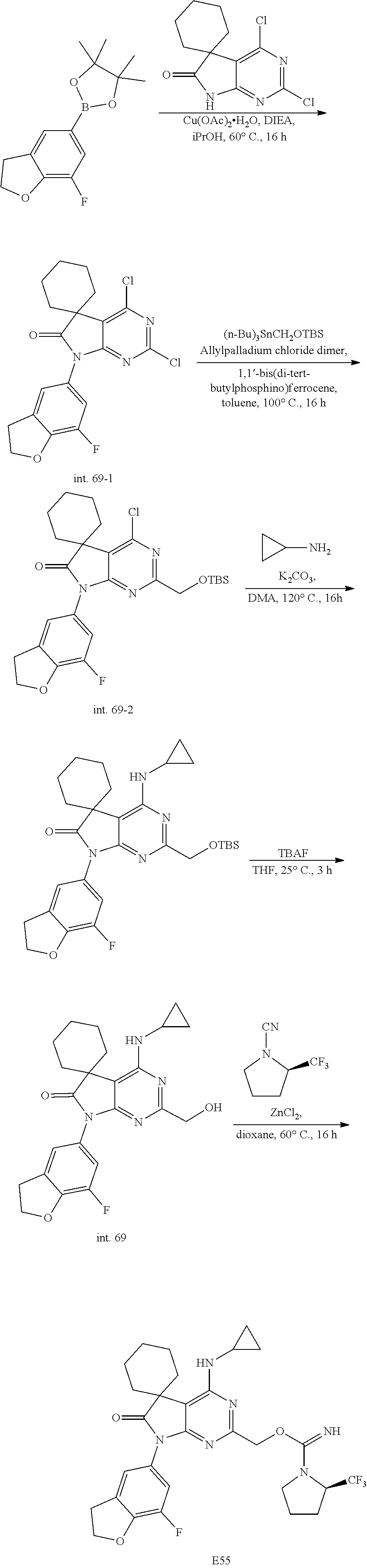

Compound E55 was synthesized as compound E53. LCMS (ESI): m/z, 589.4 [M+H]

### Example 133. Preparation of Compound E56

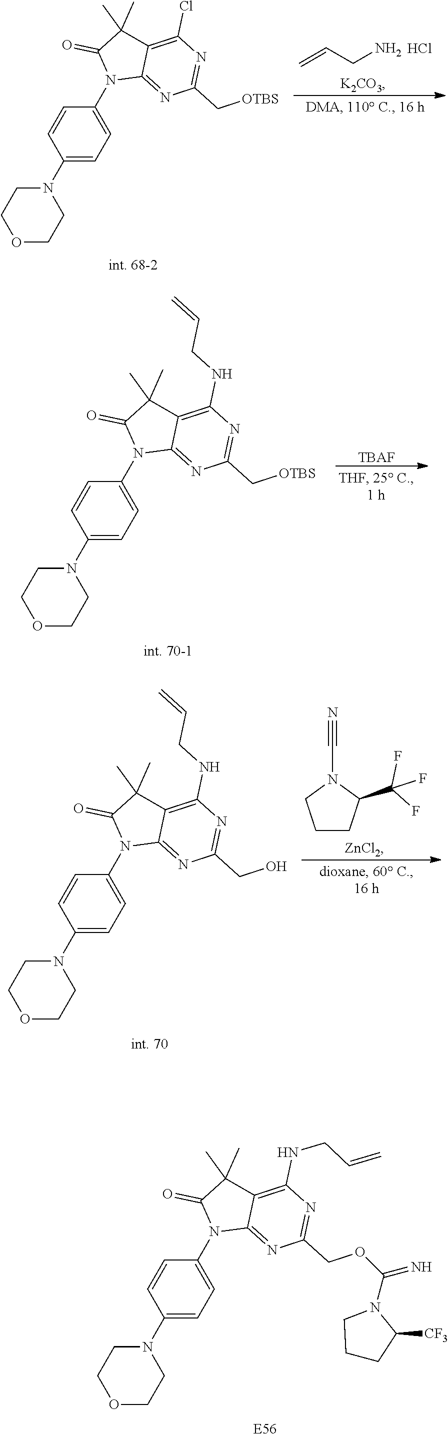

Compound E56 was synthesized as compound E22. ¹H NMR (400 MHz, CDCl₃) δ 7.36 (d, J=8.8 Hz, 2H), 6.95 (d, J=9.0 Hz, 2H), 6.96-5.96 (m, 1H), 5.25-5.14 (m, 3H), 5.01 (d, J=14.8 Hz, 1H), 4.99-4.22 (m, 2H), 4.20 (t, J=1.6 Hz, 2H), 3.89-3.84 (m, 4H), 3.50-3.38 (m, 2H), 3.22-3.16 (m, 5H), 2.11-2.01 (m, 2H), 1.95-1.80 (m, 2H), 1.50 (s, 6H). LCMS (ESI): m/z, 574.4 [M+H]⁺.

### Example 134. Preparation of Compound E57

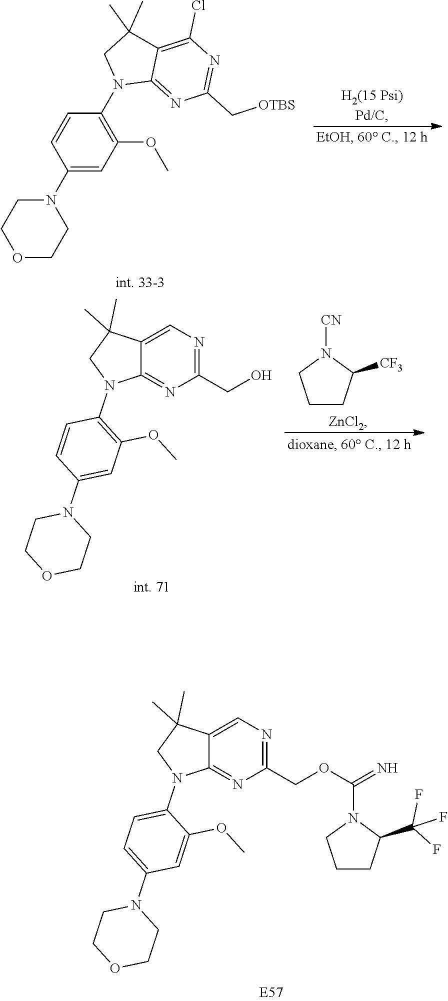

Compound E57 was synthesized as compound E58. ¹H NMR (400 MHz, CDCl₃) δ 7.94 (s, 1H), 7.28-7.25 (m, 1H), 6.50-6.48 (m, 2H), 5.17-4.95 (m, 2H), 4.55-4.47 (m, 1H), 3.88-3.86 (m, 4H), 3.79 (s, 3H), 3.68-3.63 (m, 2H), 3.47-3.40 (m, 2H), 3.18-3.16 (m, 4H), 2.06-1.99 (m, 2H), 1.90-1.83 (m, 2H), 1.39 (s, 6H). LCMS (ESI): m/z, 535.5 [M+H]⁺.

### Example 135. Preparation of Compound E58

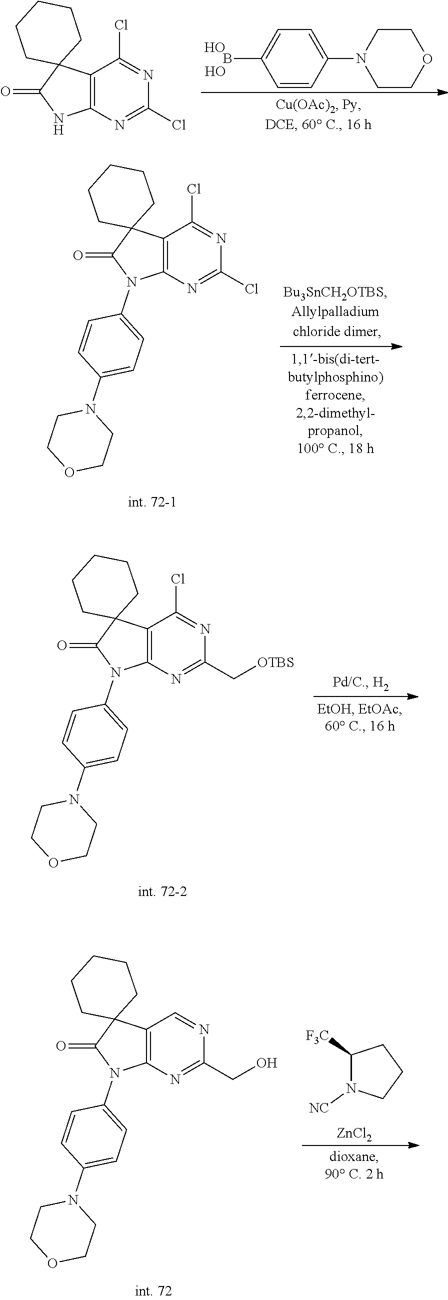

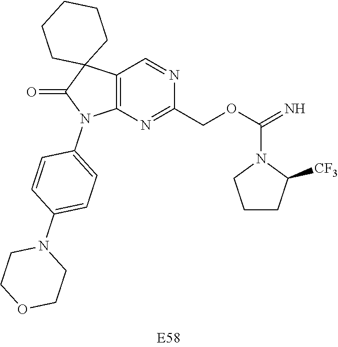 [SMILES: N=C(OCc1ncc2c(n1)N(c1ccc(N3CCOCC3)cc1)C(=O)C21CCCCC1)N1CCC[C@@H]1C(F)(F)F]

1. Synthesis of compound int. 72-2. Compound int. 72-2 was synthesized as compound int. 67-2. LCMS (ESI): m/z, 543.3 [M+H]⁺.

2. Synthesis of compound int. 72. To a solution of 2′-(((tert-butyldimethylsilyl)oxy)methyl)-4′-chloro-7′-(4-morpholinophenyl)spiro[cyclohexane-1,5′-pyrrolo[2,3-d]pyrimidin]-6′(7′H)-one (500 mg, 0.92 mmol) in EtOAc (5 mL) and EtOH (5 mL) was added Pd/C (245 mg, 2.30 mmol). The mixture was stirred at 60° C. for 16 h under H₂ (15 psi) atmosphere. The reaction was filtered and the filtrate was concentrated under reduce pressure to give 2′-(hydroxymethyl)-7′-(4-morpholinophenyl) spiro [cyclohexane-1,5′-pyrrolo[2,3-d]pyrimidin]-6′(7′H)-one (400 mg crude). LCMS (ESI): m/z, 395.2 [M+H]⁺.

3. Synthesis of compound E58. Compound E58 was synthesized as compound E15. ¹H NMR (400 MHz, CDCl₃) δ 8.63 (s, 1H), 7.39-7.32 (m, 2H), 7.01-6.95 (m, 2H), 5.41 (d, J=14.8 Hz, 1H), 5.21 (d, J=14.8 Hz, 1H), 4.46 (q, J=7.8 Hz, 1H), 3.87 (dd, J=3.6, 6.0 Hz, 4H), 3.47 (m, 2H), 3.26-3.16 (m, 4H), 1.98 (m, 10H), 1.70 (dd, J=5.2, 9.6 Hz, 4H). LCMS (ESI): 559.3 [M+H]⁺.

### Example 136. Preparation of Compound E59

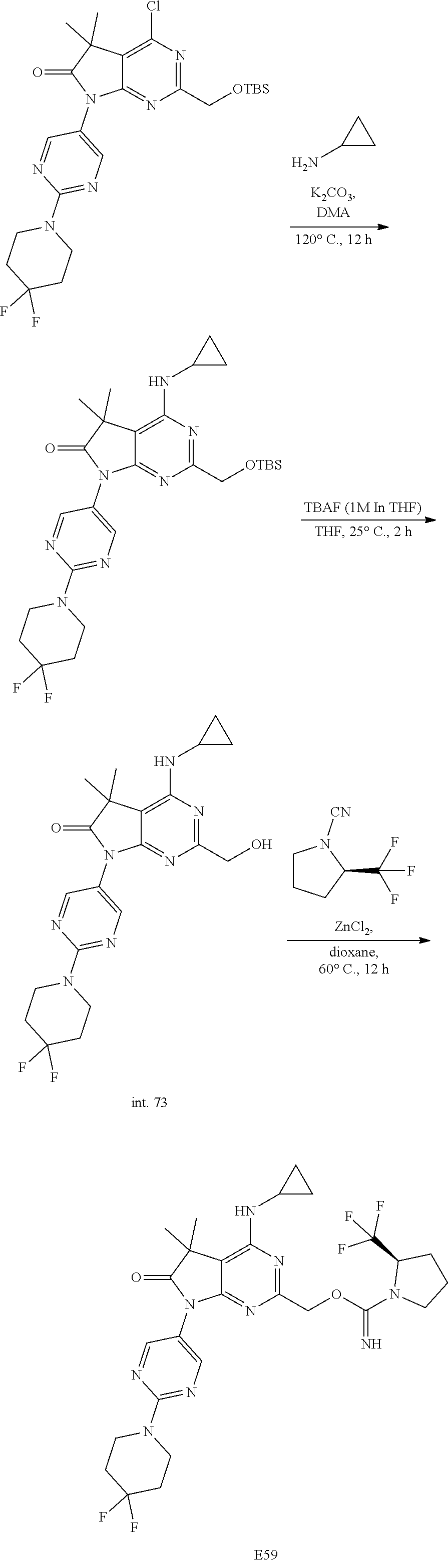

Compound E59 was synthesized as compound E22. ¹H NMR (400 MHz, CDCl₃) δ 8.48 (s, 2H), 5.25-5.07 (m, 2H), 4.61 (d, J=2.4 Hz, 1H), 4.52-4.45 (m, 1H), 4.02-3.99 (m, 4H), 3.54-3.44 (m, 2H), 2.92-2.87 (m, 1H), 2.12-1.94 (m, 8H), 1.46 (s, 6H), 0.87-0.83 (m, 2H), 0.57-0.53 (m, 2H). LCMS (ESI): m/z, 610.3 [M+H]⁺.

### Example 137. Preparation of Compound E60

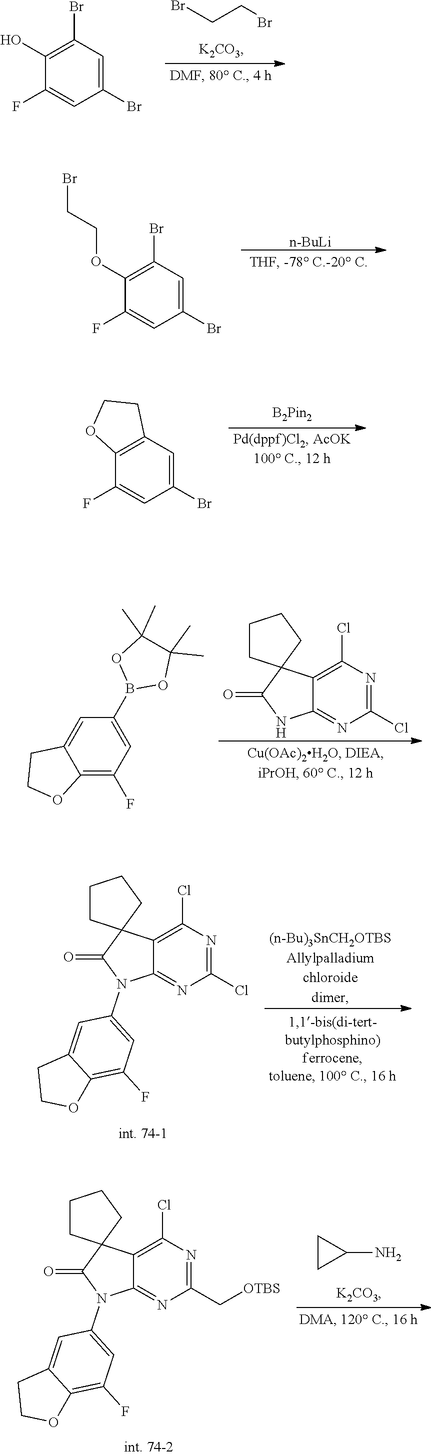

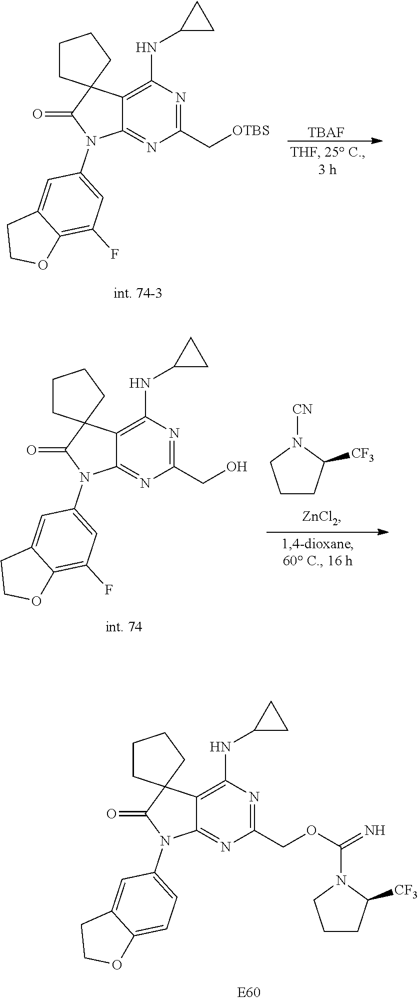

1. Synthesis of compound 1,5-dibromo-2-(2-bromoethoxy)-3-fluorobenzene. To a solution of 2,4-dibromo-6-fluorophenol (10.0 g, 37.0 mmol) and DBE (6.41 mL) in DMF (100 mL) was added K₂CO₃ (10.2 g, 74.1 mmol) at 25° C. The mixture was stirred at 80° C. for 4 h under N₂ atmosphere. The reaction mixture was quenched with water and extracted with EA. The organic layer was washed with saturated brine, dried over Na₂SO₄ and filtered. The filtrate was concentrated. The crude product was purified by column chromatography to give 1,5-dibromo-2-(2-bromoethoxy)-3-fluorobenzene (8.93 g). ¹H NMR (400 MHz, DMSO-d₆) δ 7.75 (t, J=2.0 Hz, 1H), 7.72 (dd, J=10.4, 2.4 Hz, 1H), 4.38 (d, J=5.2, 0.8 Hz, 2H), 3.77 (t, J=5.64 Hz, 2H).

2. Synthesis of compound 5-bromo-7-fluoro-2,3-dihydrobenzofuran. To a solution of 1,5-dibromo-2-(2-bromoethoxy)-3-fluorobenzene (1.0 g, 2.65 mmol) in THF (30 mL) cooled to −78° C. was added n-BuLi (1.59 mL, 2.50 M) under N₂ atmosphere. The mixture was stirred at −78° C. for 2 h and then the mixture was gradually raised to 25° C. and stirred for 1 h. The reaction solution was quenched with water and extracted with EA. The combined organic phases were washed with brine, dried over anhydrous sodium sulfate and concentrated under reduced pressure. The crude product was purified by column chromatography to afford 5-bromo-7-fluoro-2,3-dihydrobenzofuran (1.20 g). ¹H NMR (400 MHz, DMSO-d₆) δ 7.35-7.30 (m, 1H), 7.28 (d, J=1.2 Hz, 1H), 4.65 (t, J=8.8 Hz, 2H), 3.28-3.23 (m, 2H).

3. Synthesis of compound E60. Other steps of compound E60 were synthesized as compound E53. ¹H NMR (400 MHz, Chloroform-d) δ 7.10 (s, 1H), 7.07 (d, J=11.6 Hz, 1H), 5.27-5.06 (m, 2H), 4.71 (t, J=8.8 Hz, 2H), 4.54 (s, 2H), 3.56 (d, J=31.2 Hz, 2H), 3.30 (t, J=8.8 Hz, 2H), 2.91-2.89 (m, 1H), 2.14-2.09 (m, 6H), 2.00-1.83 (m, 6H), 0.94-0.92 (m, 2H), 0.56-0.52 (m, 2H).

### Example 138. Preparation of Compound E61

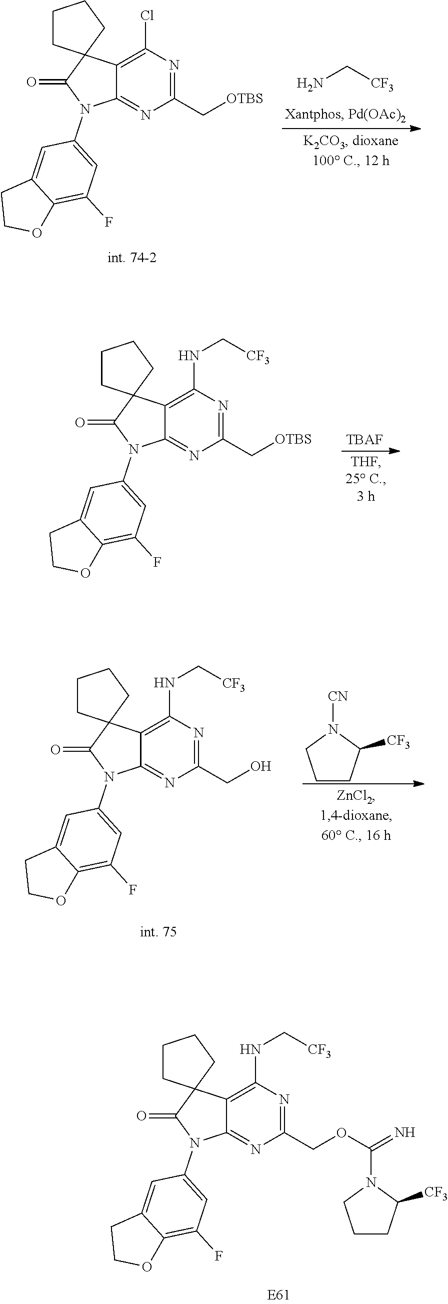

Compound E61 was synthesized as compound E50. ¹H NMR (400 MHz, CDCl₃) δ 7.10-7.05 (m, 2H), 5.24-5.09 (m, 2H), 4.71 (t, J=8.8 Hz, 2H), 4.51-4.45 (m, 2H), 4.37-4.25 (m, 2H), 3.54-3.41 (m, 2H), 3.31 (t, J=8.8 Hz, 2H), 2.22-2.09 (m, 4H), 2.04-1.85 (m, 8H). LCMS (ESI): t_{R}=3.834, m/z, 617.5 [M+H]⁺.

### Example 139. Preparation of Compound E62

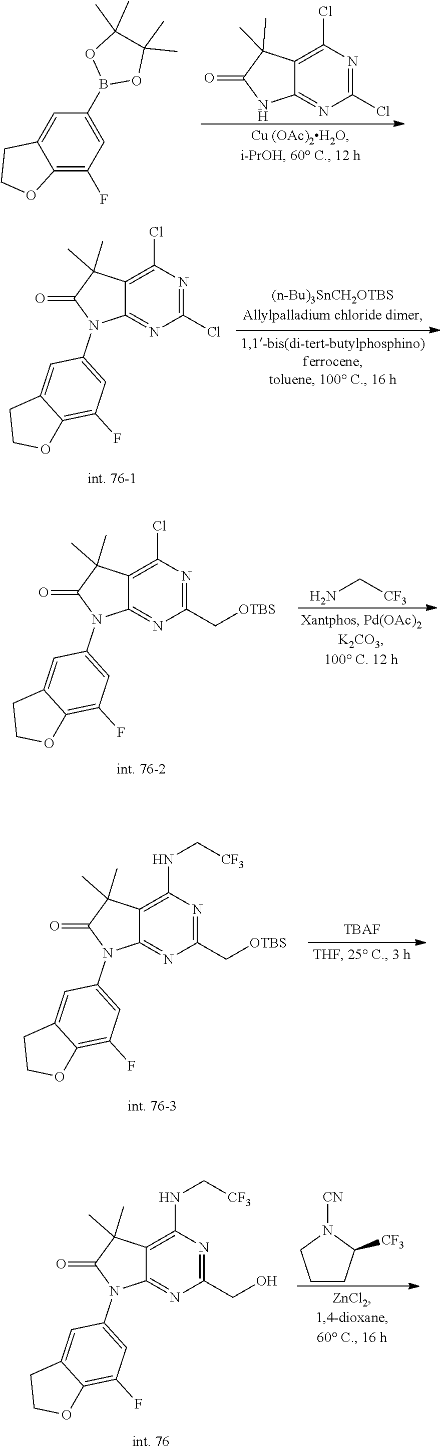

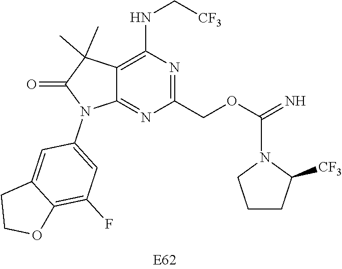

Compound E62 was synthesized as compound E50. ¹H NMR (400 MHz, DMSO-d₆) δ 7.21-7.12 (m, 3H), 5.76 (s, 1H), 4.96 (t, J=14.4 Hz, 2H), 4.70 (t, J=8.8 Hz, 2H), 4.55 (d, J=8.4 Hz, 1H), 4.27-4.23 (m, 2H), 3.29 (t, J=8.8 Hz, 4H), 1.91 (d, J=36.0 Hz, 4H), 1.45 (s, 6H).

### Example 140. Preparation of Compound E63

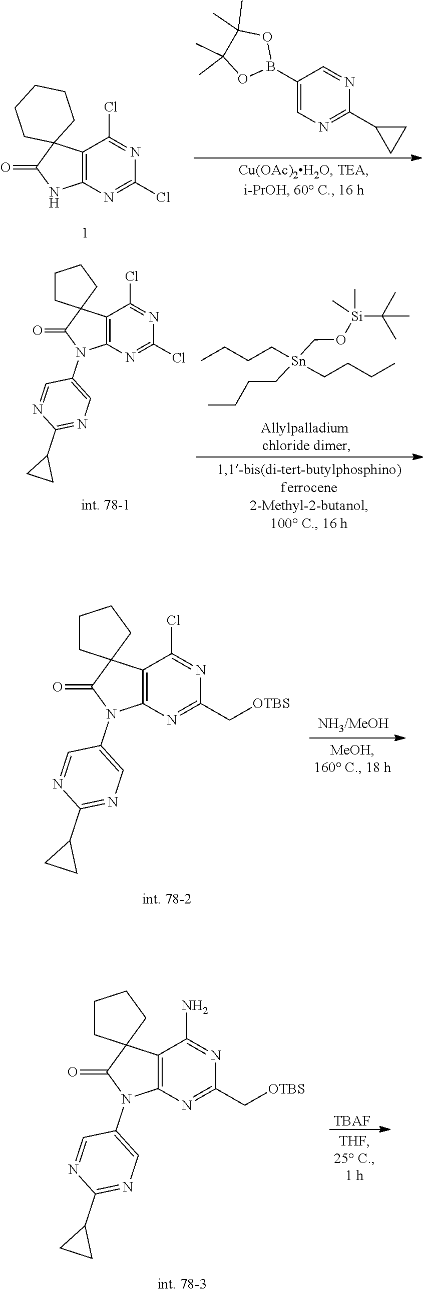

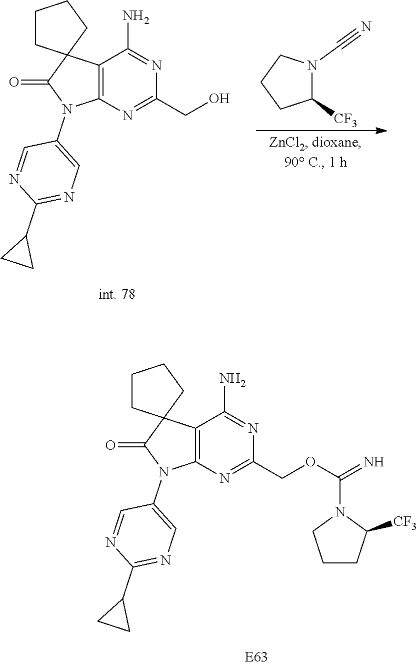 [SMILES: Nc1nc(CO)nc2c1C1(CCCC1)C(=O)N2c1cnc(C2CC2)nc1 ; N#CN1CCC[C@@H]1C(F)(F)F ; N=C(OCc1nc(N)c2c(n1)N(c1cnc(C3CC3)nc1)C(=O)C21CCCC1)N1CCC[C@@H]1C(F)(F)F]

Compound E63 was synthesized as compound E34. ¹H NMR (400 MHz, Chloroform-d) δ 8.81 (s, 2H), 5.19 (s, 1H), 4.94 (d, J=108 Hz, 4H), 4.42 (s, 1H), 3.53-3.41 (m, 2H), 2.33-2.15 (m, 5H), 2.12-1.90 (m, 8H), 1.17-1.08 (m, 4H). LCMS (ESI): m/z, 517.6 [M+H]⁺.

### Example 142. Preparation of Compound E65

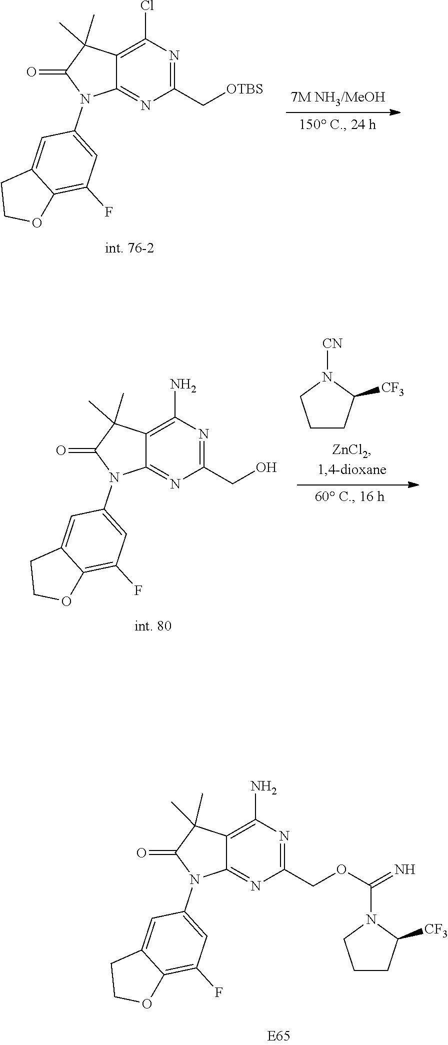 [SMILES: CC1(C)C(=O)N(c2cc(F)c3c(c2)CCO3)c2nc(CO[Si](C)(C)C(C)(C)C)nc(Cl)c21 ; CC1(C)C(=O)N(c2cc(F)c3c(c2)CCO3)c2nc(CO)nc(N)c21 ; N#CN1CCC[C@@H]1C(F)(F)F ; CC1(C)C(=O)N(c2cc(F)c3c(c2)CCO3)c2nc(COC(=N)N3CCC[C@@H]3C(F)(F)F)nc(N)c21]

Compound E65 was synthesized as compound E20. ¹H NMR (400 MHz, DMSO-d₆) δ 7.13 (dd, J=13.6, 2.4 Hz, 2H), 6.78 (s, 2H), 5.88 (s, 1H), 5.02-4.81 (m, 2H), 4.69 (t, J=8.8 Hz, 2H), 4.56-4.46 (m, 1H), 3.27 (t, J=8.8 Hz, 4H), 1.95-1.75 (m, 4H), 1.42 (s, 6H).

### Example 143. Preparation of Compound E66

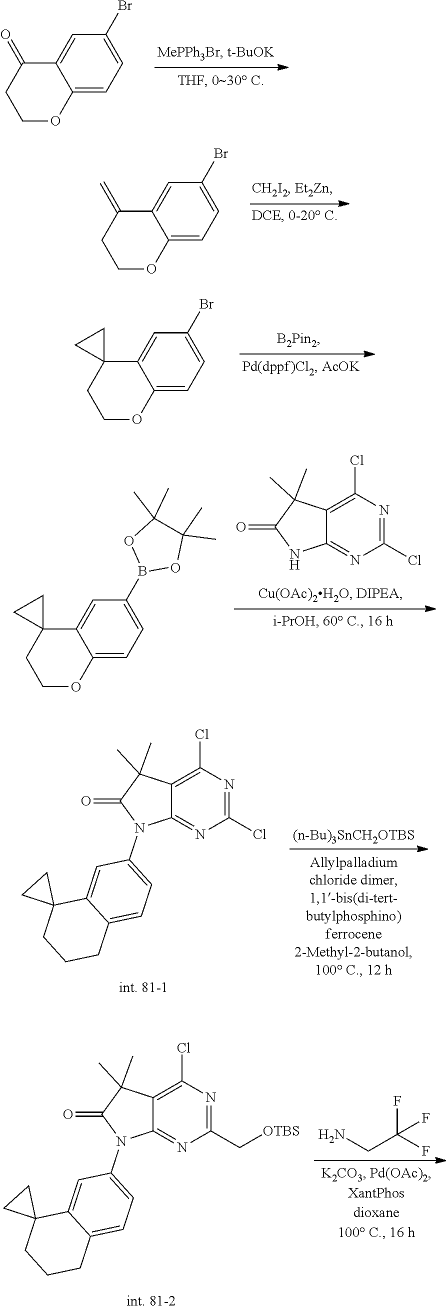

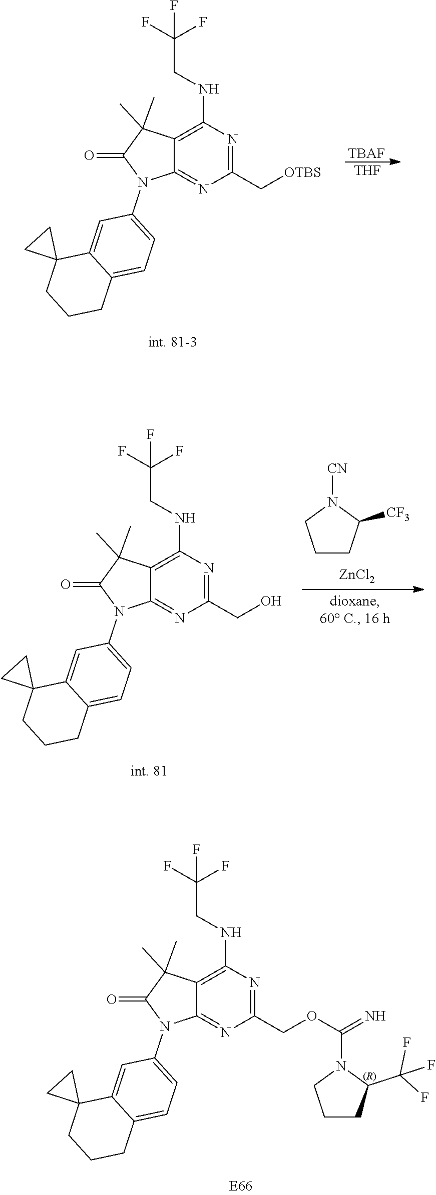

1. Synthesis of compound 6-bromo-4-methylidene-3,4-dihydro-2H-1-benzopyran. To a stirred suspension of n-BuLi (14.1 mL, 2.5 M) in THF (20 mL) at 0° C. was added methyltriphenyl phosphonium bromide (12.6 g, 35.2 mmol). The reaction was allowed to stirred for 30 min at 0° C. and then 6-bromo-2,3-dihydro-4H-chromen-4-one (2.0 g, 8.81 mmol) was added. After an additional 30 min, the ice bath was removed and the reaction was allowed to 18° C. and stirred for 15 h. The mixture was diluted with H₂O, extracted with EtOAc, washed with brine, dried and concentrated under vacuum. The crude was purified by column chromatography to give 6-bromo-4-methylidene-3,4-dihydro-2H-1-benzopyran (967 mg). ¹H NMR (400 MHz, CDCl₃) δ 3.67 (s, 3H), 2.39-2.27 (m, 4H), 1.70-1.55 (m, 4H), 1.40-1.28 (m, 6H).

2. Synthesis of compound 6-bromo-2,3-dihydrospiro[1-benzopyran-4,1′-cyclopropane]. To a solution of 6-bromo-4-methylidene-3,4-dihydro-2H-1-benzopyran (200 mg, 888 μmol) in DCM (6 mL) was added Et₂Zn (4.44 mL, 1 M) at 20° C., CH₂1₂ (717 μL) was then added dropwise. The reaction mixture was stirred at 20° C. for 16 h. The reaction was diluted with DCM (10 mL), washed with saturated aq. NH₄Cl (5 mL) and water, dried over sodium sulfate, filtered and concentrated under vacuum. The crude was purified by column chromatography to give 6-bromo-2,3-dihydrospiro[1-benzopyran-4,1′-cyclopropane] (100 mg). ¹H NMR (400 MHz, Chloroform-d) δ 7.11 (dd, J=8.6, 2.4 Hz, 1H), 6.76-6.64 (m, 2H), 4.32-4.23 (m, 2H), 1.88-1.79 (m, 2H), 1.12-0.95 (m, 2H), 0.92-0.79 (m, 2H).

3. Synthesis of compound 2-{2,3-dihydrospiro[1-benzopyran-4,1′-cyclopropan]-6-yl}-4,4,5,5-tetramethyl-1,3,2-dioxaborolane. To a mixture of (PinB)₂ (1.34 g, 5.28 mmol) and 1,4-dioxane (20 mL), 6-bromo-2,3-dihydrospiro[1-benzopyran-4,1′-cyclopropane](1.05 g, 4.40 mmol), (dppf)₂PdCl₂ (160 mg, 220 μmol), and KOAc (864 mg, 8.81 mmol) were added in turn. The mixture was degassed under vacuum and purged with N₂ several times. The reaction mixture was stirred at 90° C. for 16 h. After cooled to 25° C., the reaction mixture was diluted with H₂O and extracted with EA. The organic layer was washed with brine. The solution was dried over Na₂SO₄ and filtered. The filtrate was evaporated. The crude product was purified by column chromatography to give 2-{2,3-dihydrospiro[1-benzopyran-4,1′-cyclopropan]-6-yl}-4,4,5,5-tetramethyl-1,3,2-dioxaborolane (1.03 g crude). ¹H NMR (400 MHz, Chloroform-d) δ 7.50 (dd, J=8.1, 1.6 Hz, 1H), 7.12 (d, J=1.6 Hz, 1H), 6.80 (d, J=8.1 Hz, 1H), 4.36-4.24 (m, 2H), 1.85 (d, J=10.3 Hz, 2H), 1.31 (s, 12H), 1.18-1.13 (m, 2H), 0.83-0.80 (m, 2H).

4. Synthesis of compound E66. Other steps of compound E66 were synthesized as compound E21. LCMS (ESI): m/z, 613.3 [M+H]⁺.

### Example 144. Preparation of Compound E67

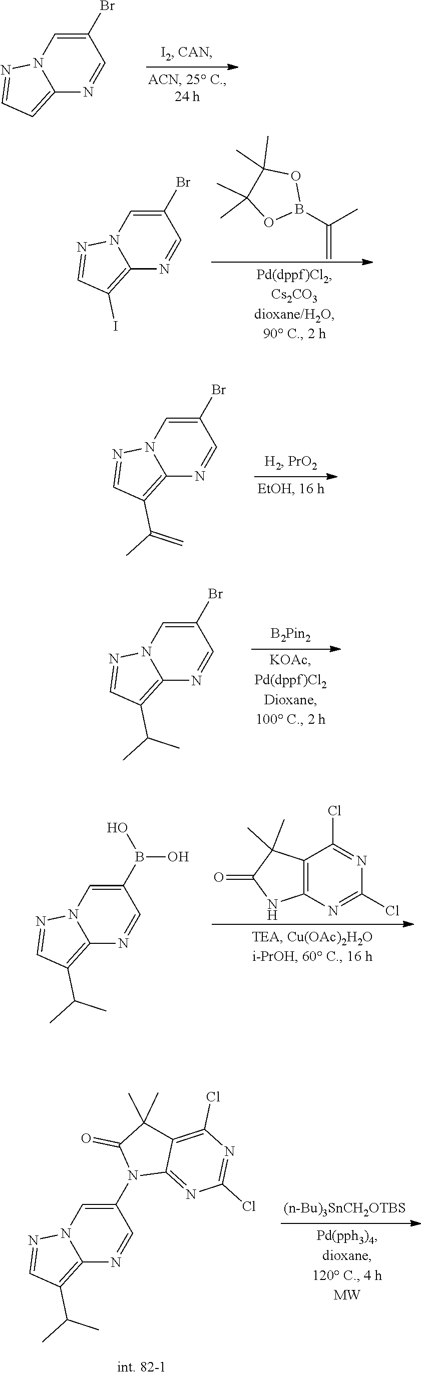

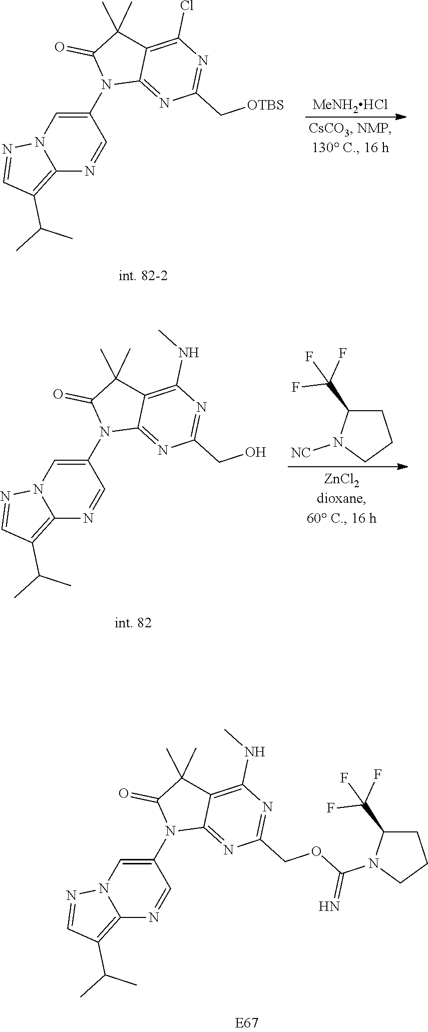 [SMILES: "MeNH2·HCl" ; CC(C)c1cnn2cc(N3C(=O)C(C)(C)c4c(Cl)nc(CO[Si](C)(C)C(C)(C)C)nc43)cnc12 ; CNc1nc(CO)nc2c1C(C)(C)C(=O)N2c1cnc2c(C(C)C)cnn2c1 ; N#CN1CCC[C@@H]1C(F)(F)F ; CNc1nc(COC(=N)N2CCC[C@@H]2C(F)(F)F)nc2c1C(C)(C)C(=O)N2c1cnc2c(C(C)C)cnn2c1]

1. Synthesis of compound 6-bromo-3-iodopyrazolo[1,5-a]pyrimidine. A mixture of 6-bromopyrazolo[1,5-a]pyrimidine (5.0 g, 25.2 mmol), CAN (8.03 g, 15.1 mmol), 12 (3.85 g, 15.1 mmol) in ACN (150 mL) was stirred at 25° C. for 24 h under N₂ atmosphere. The mixture was filtered. The filtrate was concentrated and the crude was dissolved with DCM (500 mL). The mixture was filtered. The filtrate was concentrated under reduced pressure to afford crude product. The filter cake was dissolved with H₂O and the aqueous phase was extracted with DCM. Then combined organic layers were washed with water and dried over Na₂SO₄. The organic solution was concentrated to afford crude product. The all crude products were purified by column chromatography to afford 6-bromo-3-iodopyrazolo[1,5-a]pyrimidine (7.23 g). ¹H NMR (400 MHz, DMSO-d₆) δ 9.64 (d, J=2.0 Hz, 1H), 8.67 (d, J=2.0 Hz, 1H), 8.33 (s, 1H).

2. Synthesis of compound 6-bromo-3-(prop-1-en-2-yl)pyrazolo[1,5-a]pyrimidine. To a solution of 6-bromo-3-iodopyrazolo[1,5-a]pyrimidine (5.0 g, 15.4 mmol), 4,4,5,5-tetramethyl-2-(prop-1-en-2-yl)-1,3,2-dioxaborolane (2.85 g, 17.0 mmol), Cs₂CO₃ (10.1 g, 30.9 mmol), Pd(dppf)Cl₂ (1.13 g, 1.54 mmol) and H₂O (30 mL) in dioxane (120 mL) was stirred at 90° C. for 2 h under N₂ atmosphere. The mixture solution was concentrated. The crude product was purified by column chromatography to afford 6-bromo-3-(prop-1-en-2-yl)pyrazolo[1,5-a]pyrimidine (2.6 g). ¹H NMR (400 MHz, DMSO-d₆) δ 9.59 (d, J=2.4 Hz, 1H), 8.67 (d, J=2.4 Hz, 1H), 8.34 (s, 1H), 5.91-5.90 (m, 1H), 5.09-5.08 (m, 1H), 2.19 (s, 3H).

3. Synthesis of compound 6-bromo-3-isopropylpyrazolo[1,5-a]pyrimidine. To a solution of 6-bromo-3-(prop-1-en-2-yl)pyrazolo[1,5-a]pyrimidine (3.73 g, 15.67 mmol) in EtOH (70 mL) was added PtO₂ (427 mg, 1.88 mmol) and stirred at 25° C. for 16 h under H₂ (15 psi) atmosphere. The reaction mixture was filtered. The filtrate was concentrated to afford 6-bromo-3-isopropylpyrazolo[1,5-a]pyrimidine (3.0 g).

¹H NMR (400 MHz, DMSO-d₆) δ 9.48 (d, J=2.0 Hz, 1H), 8.53 (d, J=2.4 Hz, 1H), 8.11 (s, 1H), 3.21 (d, J=7.2 Hz, 1H), 1.32 (d, J=6.8 Hz, 6H).

4. Synthesis of compound E67. Other steps of compound E67 were synthesized as compound E31. ¹H NMR (400 MHz, DMSO-d₆) δ 9.30 (d, J=2.4 Hz, 1H), 8.57 (d, J=2.0 Hz, 1H), 8.21 (s, 1H), 6.84 (d, J=4.8 Hz, 1H), 5.81 (s, 1H), 5.06-4.95 (m, 2H), 4.52-4.48 (m, 1H), 3.35-3.25 (m, 3H), 2.91 (d, J=4.4 Hz, 3H), 1.79-1.71 m, 2H), 1.69-1.64 (m, 2H), 1.48 (s, 6H), 1.35 (d, J=7.2, 6H).

### Example 145. Preparation of Compound E68

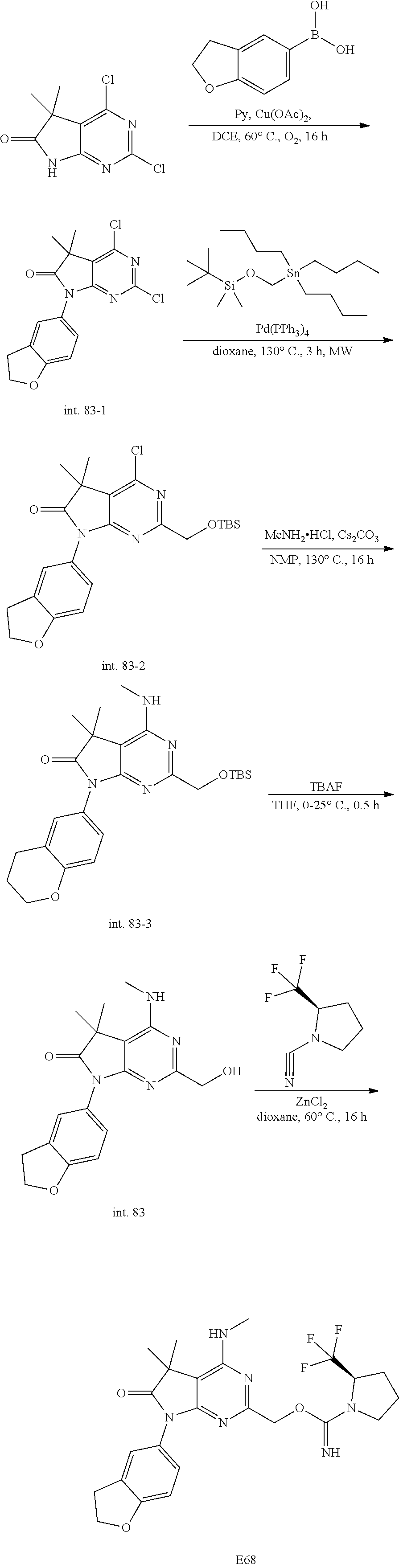

Compound E68 was synthesized as compound E23. ¹H NMR (400 MHz, CDCl₃) δ 7.23 (d, J=2.2 Hz, 1H), 7.13 (dd, J=8.4, 2.2 Hz, 1H), 6.82 (d, J=8.4 Hz, 1H), 5.22 (d, J=14.6 Hz, 1H), 5.05 (d, J=14.6 Hz, 1H), 4.61 (t, J=8.8 Hz, 2H), 4.54-4.42 (m, 2H), 3.54-3.41 (m, 2H), 3.24 (t, J 8.8 Hz, 2H), 3.07 (d, J=4.8 Hz, 3H), 2.08-1.83 (m, 4H), 1.48 (s, 6H). LCMS (ESI): m/z, 505.3 [M+H]⁺.

### Example 146. Preparation of Compound E69

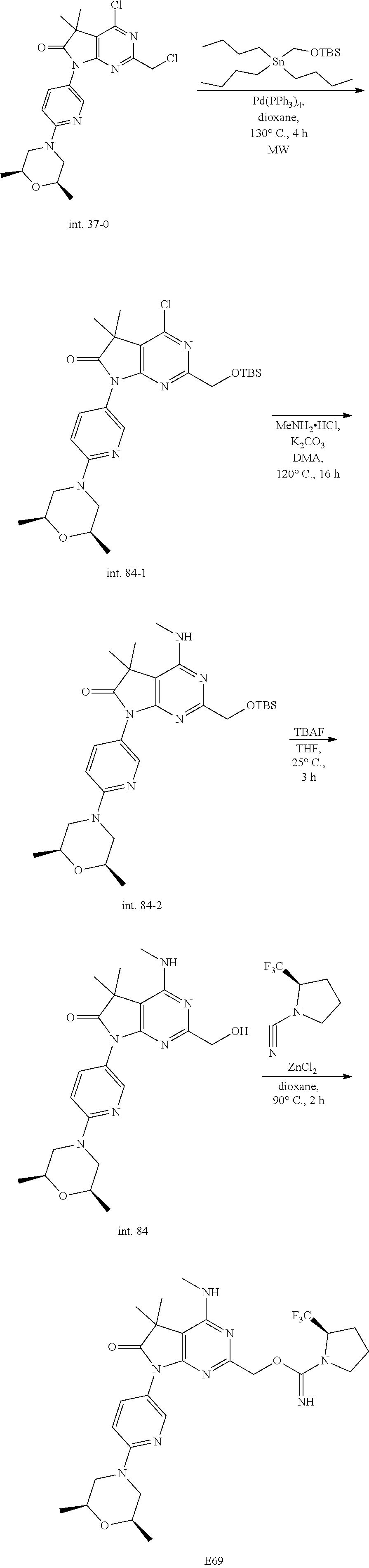

Compound E69 was synthesized as compound E23. LCMS (ESI): m/z, 577.3 [M+H]. ¹H NMR (400 MHz, Chloroform-c) ¹H NMR (400 MHz, Chloroform-d) δ 8.28-8.29 (m, 1H), 7.58-7.61 (m, 1H), 6.66-6.68 (m, 1H), 5.19-5.22 (m, 1H), 5.02-5.06 (m, 1H), 4.52-4.40 (m, 2H), 4.06-4.10 (m, 2H), 3.68-3.73 (m, 2H), 3.45-3.50 (m, 2H), 3.07 (d, J=4.8 Hz, 3H), 2.55-2.51 (m, 2H), 2.10-2.03 (m, 2H), 1.90-1.95 (m, 2H), 1.48 (s, 6H), 1.27 (d, J=6.4 Hz, 6H).

### Example 147. Preparation of Compound E70

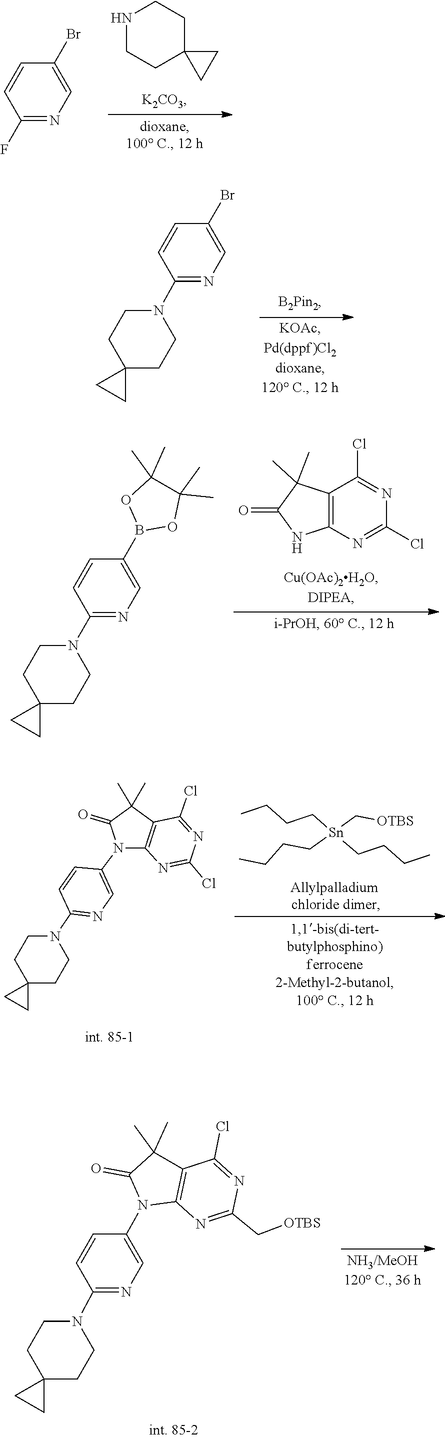

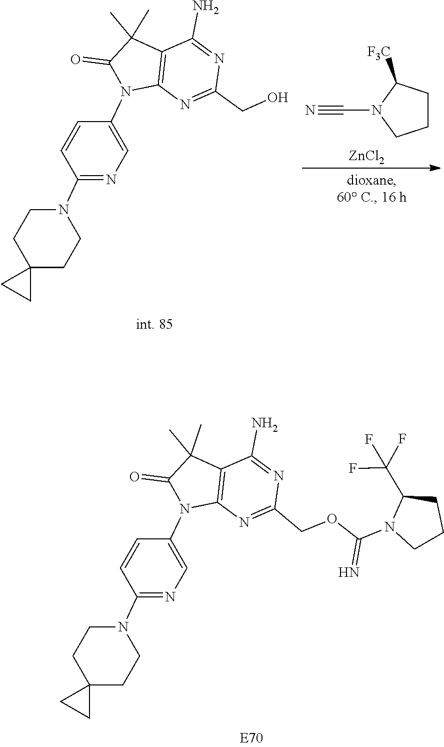

Compound E70 was synthesized as compound E34 and E46. LCMS (ESI): m/z, 559.2 [M+H]⁺. ¹H NMR (400 MHz, CDCl₃) δ 8.06 (d, J=2.4 Hz, 1H), 7.50 (dd, J=2.7, 9.1 Hz, 1H), 6.91 (d, J=9.2 Hz, 1H), 6.77 (s, 2H), 5.05-4.81 (m, 2H), 4.52 (s, 1H), 3.64 (t, J=5.6 Hz, 4H), 2.00 (q, J=6.4, 7.2, Hz, 1H), 1.91-1.71 (m, 4H), 1.40 (d, J=14.4 Hz, 9H), 1.23 (s, 4H), 0.36 (s, 3H).

### Example 148. Preparation of Compound E71

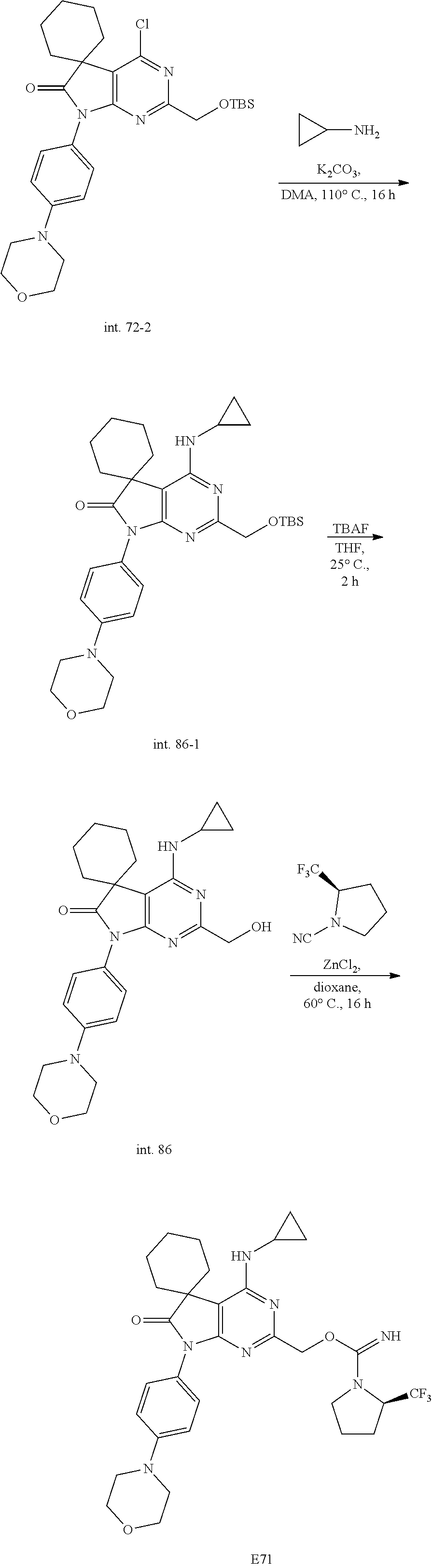

Compound E71 was synthesized as compound E22. ¹H NMR (400 MHz, CDCl₃) δ 7.34-7.31 (d, J=16.0 Hz, 2H), 6.97-6.93 (d, J=16.0 Hz, 2H), 5.23 (d, J=16.0 Hz, 1H), 5.04 (d, J=16.0 Hz, 1H), 4.65 (s, 1H), 4.57-4.47 (m, 1H), 3.87 (dd, J=5.0, 3.2 Hz, 4H), 3.49 (dd, J=10.0, 20.0 Hz, 2H), 3.20-3.16 (m, 4H), 2.88 (dd, J=4.0, 8.0 Hz, 1H), 2.26-2.18 (m, 2H), 2.09-2.03 (m, 2H), 1.80 (d, J=6.4 Hz, 4H), 1.64 (d, J=10.0 Hz, 2H), 0.88-0.85 (m, 3H), 0.56 (dd, J=2.0, 4.4 Hz, 2H). LCMS (ESI): m/z, 614.3 [M+H]⁺.

### Example 149. Preparation of Compound E72

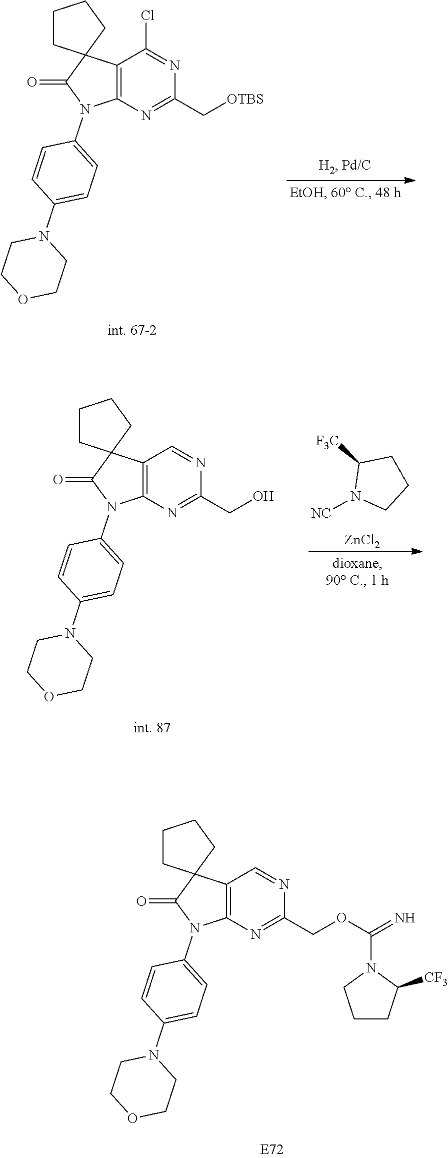

Compound E46 was synthesized as compound E57. ¹H NMR (400 MHz, CDCl₃) δ 8.32 (s, 1H), 7.39 (d, J=9.2 Hz, 2H), 6.98 (d, J=9.2 Hz, 2H), 5.42-5.15 (m, 2H), 4.45 (q, J=7.8 Hz, 1H), 3.94-3.82 (m, 4H), 3.55-3.36 (m, 2H), 3.27-3.15 (m, 4H), 2.35-2.26 (m, 2H), 2.13-1.79 (m, 10H). LCMS (ESI): 545.3 [M+H]⁺.

### Example 150. Preparation of Compound E73

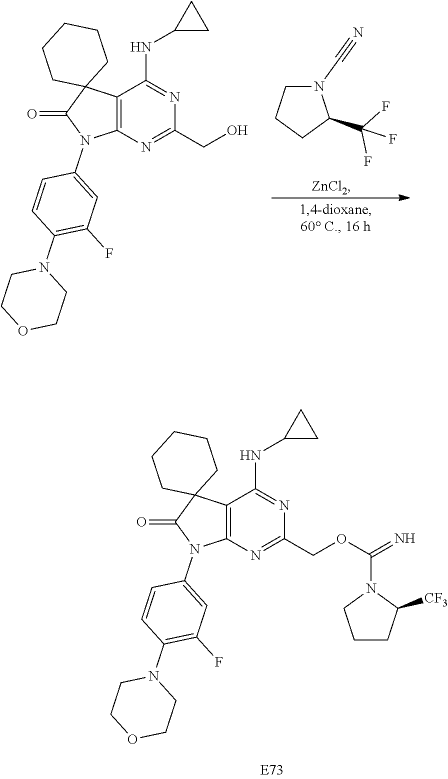

Compound E73 was synthesized as compound E16. LCMS (ESI): m/z, 632.4 [M+H]⁺.

### Example 151. Preparation of Compound E74

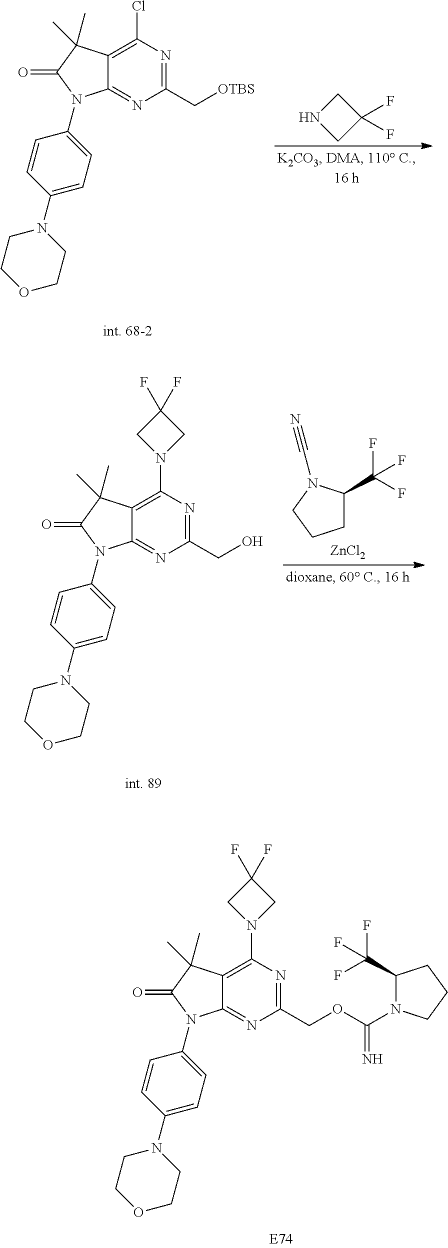

Compound E74 was synthesized as compound E17. ¹H NMR (400 MHz, CDCl3) δ 7.32 (d, J=8.8 Hz, 2H), 6.98 (d, J=8.4 Hz, 2H), 5.29 (d, J=14.6 Hz, 1H), 5.10 (d, J=14.6 Hz, 1H), 4.56 (t, J=12.0 Hz, 5H), 3.90-3.83 (m, 4H), 3.59-3.44 (m, 2H), 3.23-3.16 (m, 4H), 2.07 (d, J=9.6 Hz, 2H), 1.99-1.70 (m, 4H), 1.50 (d, J=1.2 Hz, 6H). LCMS (ESI): m/z, 610.3 [M+H]⁺.

### Example 152. Preparation of Compound E75

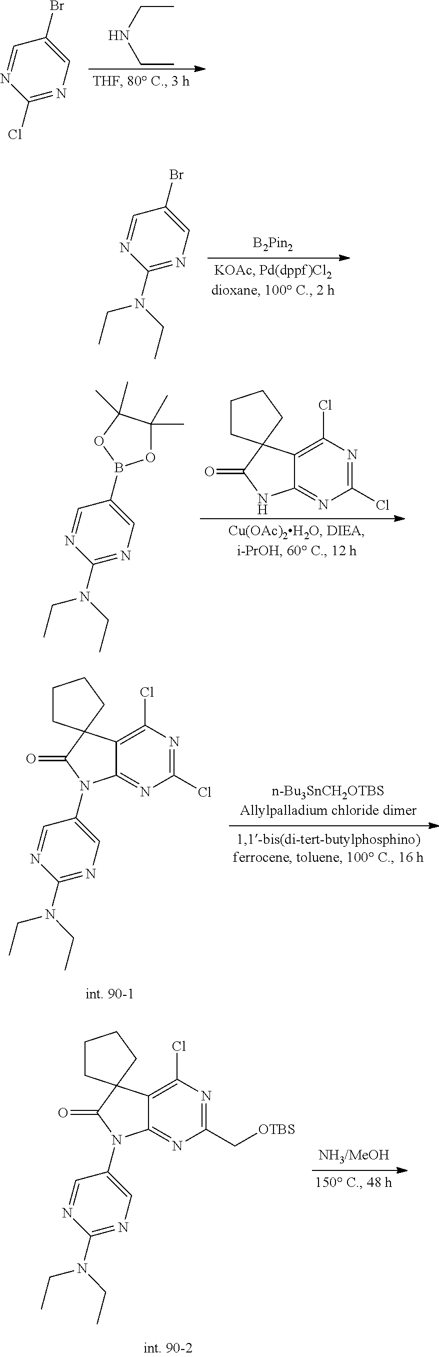

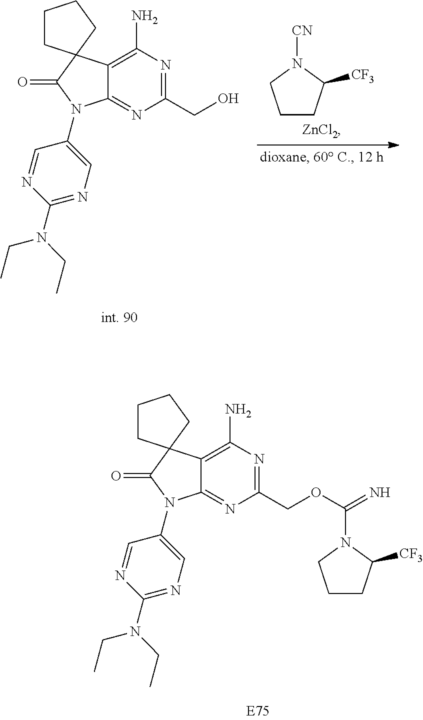 [SMILES: CCN(CC)c1ncc(N2C(=O)C3(CCCC3)c3c(N)nc(CO)nc32)cn1 ; N#CN1CCC[C@@H]1C(F)(F)F ; CCN(CC)c1ncc(N2C(=O)C3(CCCC3)c3c(N)nc(COC(=N)N4CCC[C@@H]4C(F)(F)F)nc32)cn1]

Compound E75 was synthesized as compound E34 and E46. ¹H NMR (400 MHz, CDCl₃) δ 8.38 (s, 2H), 5.19-4.99 (m, 2H), 4.77 (s, 2H), 4.47-4.42 (m, 1H), 3.64 (q, J=7.2 Hz, 4H), 3.47-3.41 (m, 2H), 2.19-1.91 (m, 12H), 1.20 (t, J=7.2 Hz, 6H). LCMS (ESI): m/z, 548.4 [M+H]⁺.

### Example 153. Preparation of Compound E76

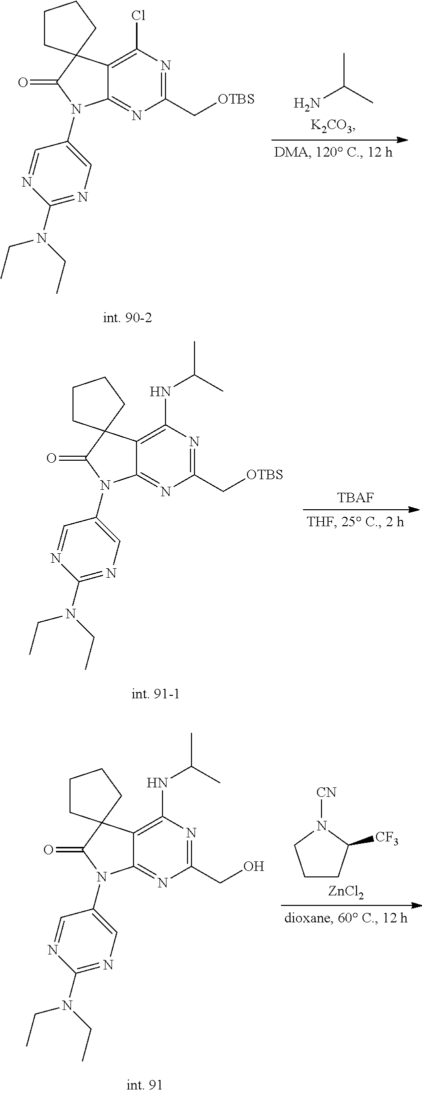

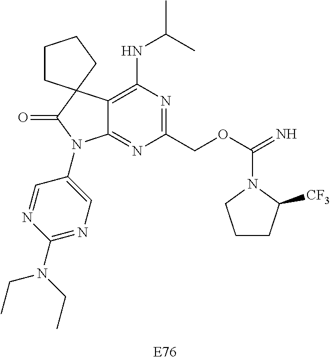 [SMILES: CCN(CC)c1ncc(N2C(=O)C3(CCCC3)c3c(NC(C)C)nc(COC(=N)N4CCC[C@@H]4C(F)(F)F)nc32)cn1]

Compound E76 was synthesized as compound E22. ¹H NMR (400 MHz, CDCl₃) δ 8.38 (s, 2H), 5,20-4.99 (m, 2H), 4.77 (s, 2H), 4.49-4.36 (m, 2H), 4.07-4.05(m, 1H), 3.64 (q, J=7.2 Hz, 4H), 3.49-3.41 (m, 2H), 2.18-1.87 (m, 12H), 1.25-1.18 (m, 12H). LCMS (ESI): m/z, 590.7 [M+H]⁺

### Example 154. Preparation of Compound E77

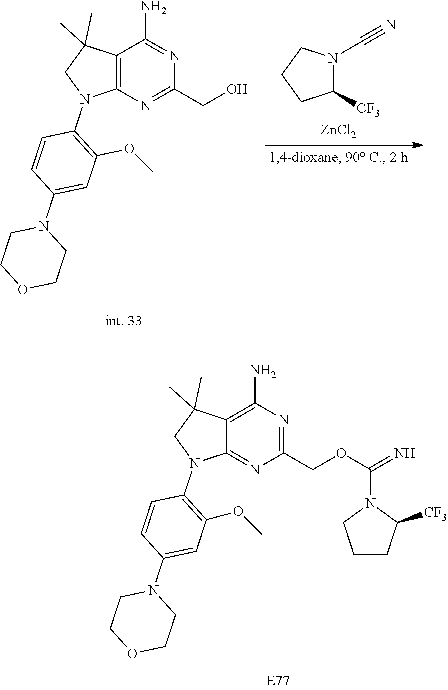

Compound E77 was synthesized as compound E15. ¹H NMR (400 MHz, Chloroform-d) δ 7.22 (d, J=8.8 Hz, 1H), 6.49 (d, J=9.6 Hz, 2H), 5.05 (d, J=14.0 Hz, 1H), 4.89 (d, J=13.2 Hz, 1H), 4.67 (s, 1H), 4.57-4.51 (m, 1H), 3.91-3.83 (m, 4H), 3.78 (s, 3H), 3.63-3.56 (m, 2H), 3.55-3.47 (m, 2H), 3.20-3.12 (m, 4H), 2.06 (d, J=8.4 Hz, 4H), 1.41 (s, 6H). LCMS (ESI): m/z, 550.3 [M+H]⁺.

### Example 155. Preparation of Compound E78

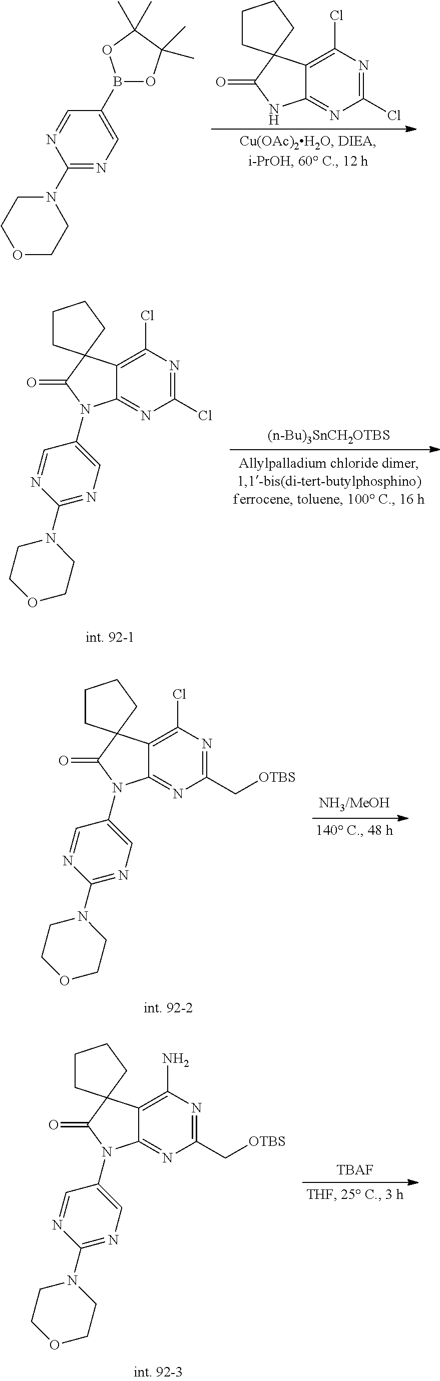

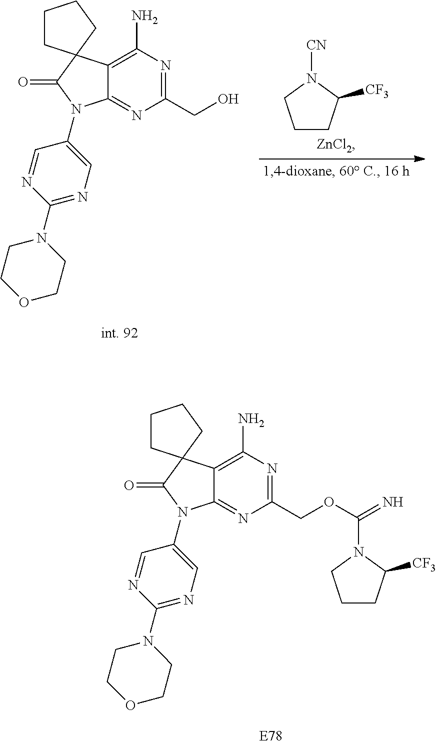

Compound E78 was synthesized as compound E34. ¹H NMR (400 MHz, CDCl₃) δ 8.46 (s, 2H), 5.21-5.01 (m, 2H), 4.76 (s, 2H), 4.45 (t, J=7.6 Hz, 1H), 3.84 (dd, J=3.6, 5.2 Hz, 4H), 3.77 (t, J=4.4 Hz, 4H), 3.47 (dt, J=8.0, 17.6 Hz, 2H), 2.21-2.04 (m, 8H), 2.02-1.88 (m, 4H). LCMS (ESI): m/z, 562.3 [M+H]⁺.

### Example 156. Preparation of Compound E79

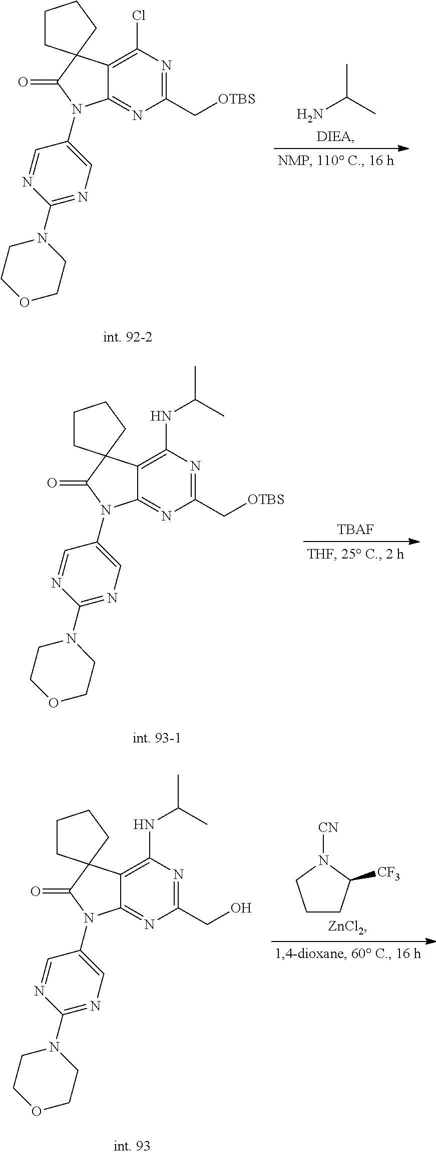

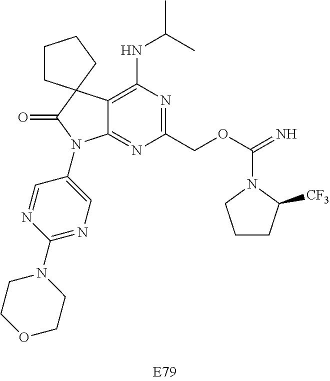 [SMILES: CC(C)Nc1nc(COC(=N)N2CCC[C@@H]2C(F)(F)F)nc2c1C1(CCCC1)C(=O)N2c1cnc(N2CCOCC2)nc1]

Compound E79 was synthesized as compound E22. LCMS (ESI): m/z, 604.3 [M+H]; H NMR (400 MHz, DMSO-d₆) δ 8.42 (s, 2H), 5.75-5.73 (m, 2H), 5.02-4.90 (m, 2H), 4.57-4.39 (m, 2H), 3.75-3.66 (m, 8H), 2.33-2.16 (m, 2H), 2.07-1.86 (m, 10H), 1.19 (d, J=6.8 Hz, 6H).

### Example 157. Preparation of Compound E80

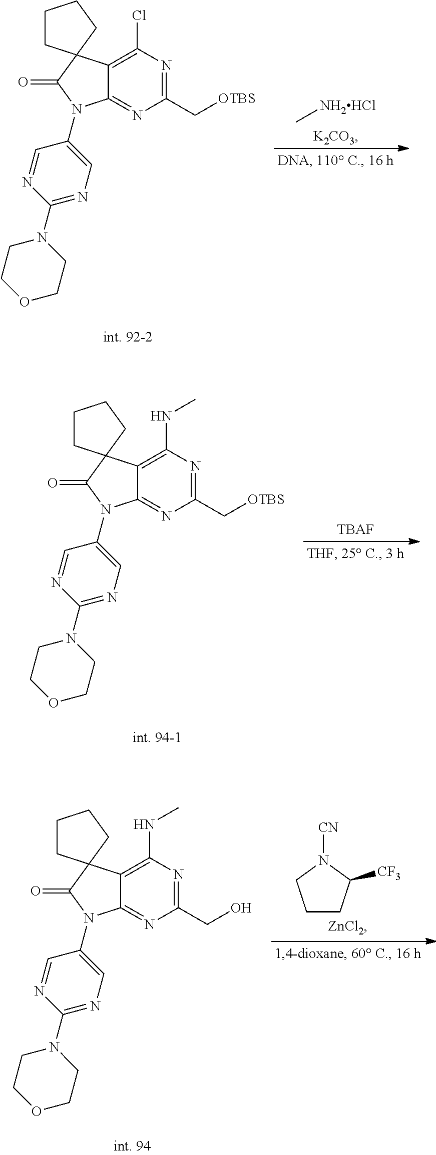

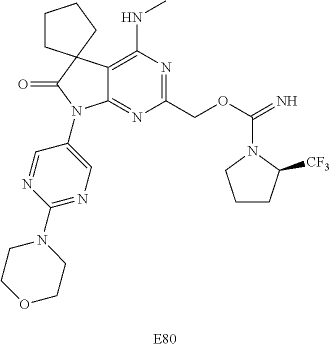 [SMILES: CNc1nc(COC(=N)N2CCC[C@@H]2C(F)(F)F)nc2c1C1(CCCC1)C(=O)N2c1cnc(N2CCOCC2)nc1]

Compound E80 was synthesized as compound E22. ¹H NMR (400 MHz, CDCl₃) δ 8.46 (s, 2H), 5.20 (d, J=14.8 Hz, 1H), 5.05 (d, J=14.8 Hz, 1H), 4.48 (1H), 4.28 (q, J=4.8 Hz, 1H), 3.85-3.81 (m, 4H), 3.78-3.74 (m, 4H), 3.48 (m, 2H), 3.08 (d, J=4.8 Hz, 3H), 2.16-1.89 (m, 11H). LCMS (ESI): m/z, 576.2 [M+H]⁺.

### Example 158. Preparation of Compound E81

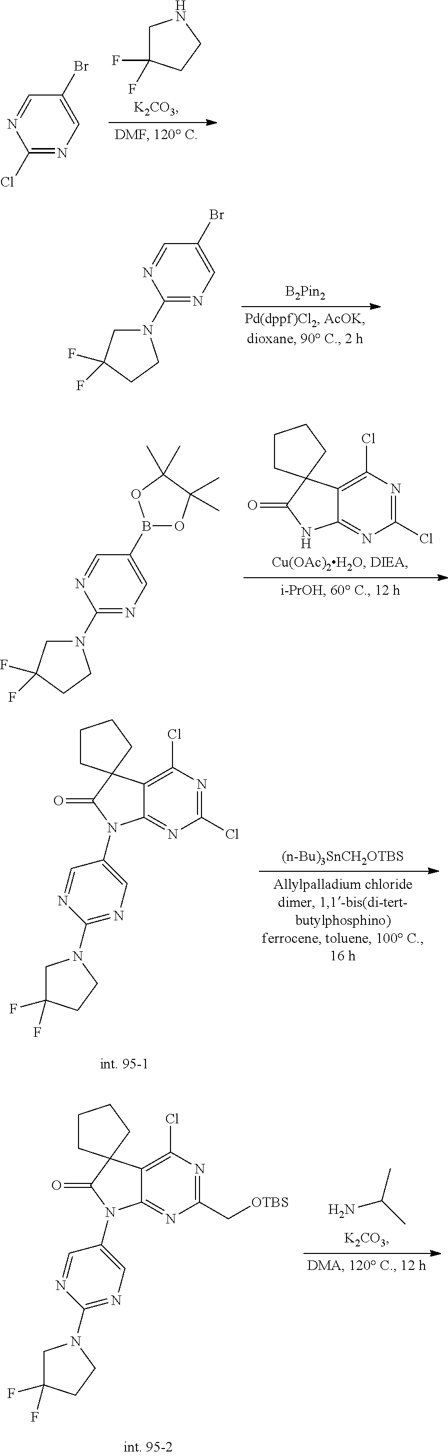

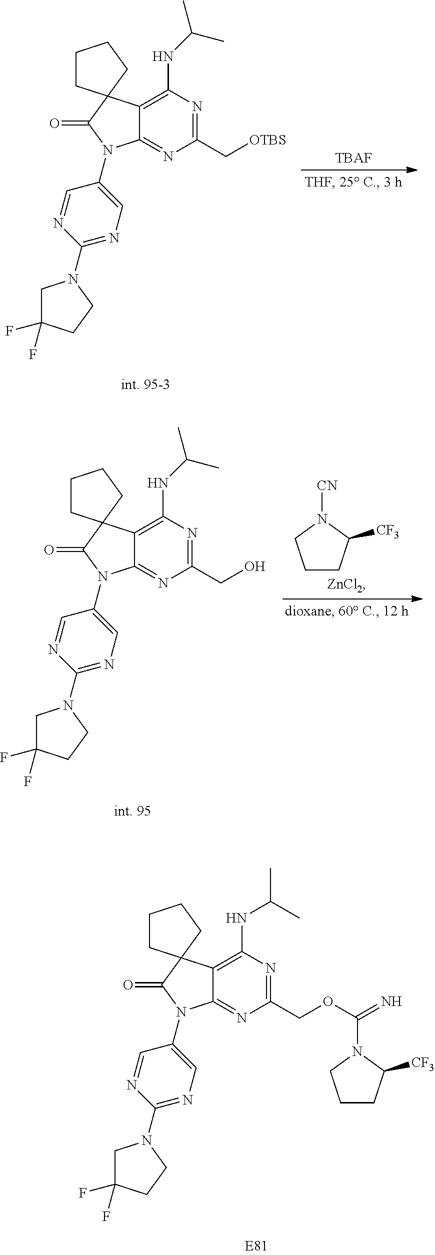

Compound E81 was synthesized as compound E60. ¹H NMR (400 MHz, CDCl₃) δ 8.49 (s, 2H), 5.18-5.00 (m, 2H), 4.48-4.34 (m, 1H), 4.09-4.07 (m, 1H), 3.96 (t, J=13.2 Hz, 2H), 3.84 (t, J=7.6 Hz, 2H), 3.54-3.44 (m, 2H), 2.54-2.43 (m, 2H), 2.19-1.65 (m, 12H), 1.24 (d, J=6.4 Hz, 6H). LCMS (ESI): m/z, 624.6 [M+H]⁺.

### Example 159. Preparation of Compound E82

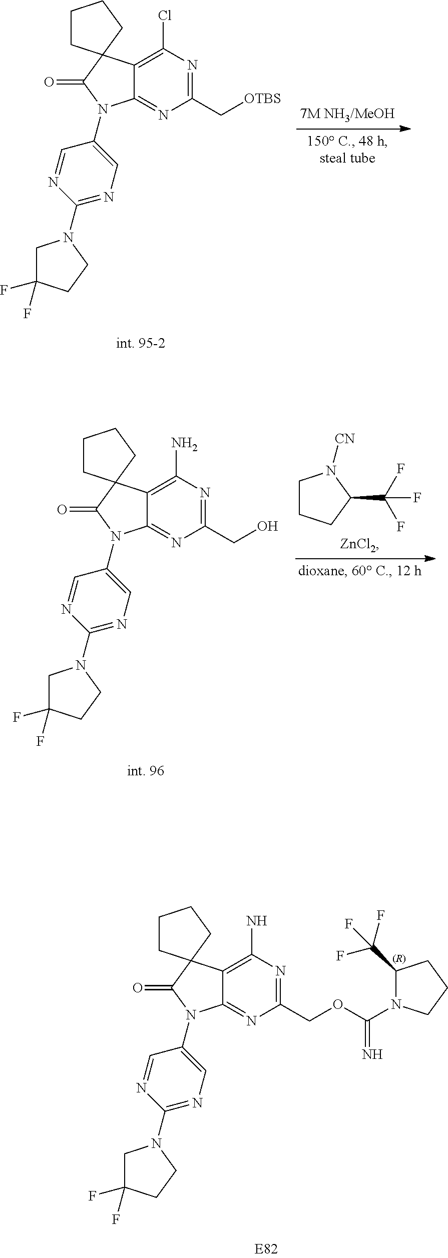

Compound E82 was synthesized as compound E20. ¹H NMR (400 MHz, CDCl₃) δ 8.49 (s, 2H), 5.19-5.04 (m, 2H), 4.79 (s, 2H), 4.44-4.41 (m, 1H), 3.96 (t, J=13.2 Hz, 2H), 3.85 (t, J=7.6 Hz, 2H), 3.51-3.42 (m, 2H), 2.54-2.44 (m, 2H), 2.18-1.92 (m, 12H). LCMS (ESI): m/z, 582.6 [M+H]⁺

### Example 160. Preparation of Compound E83

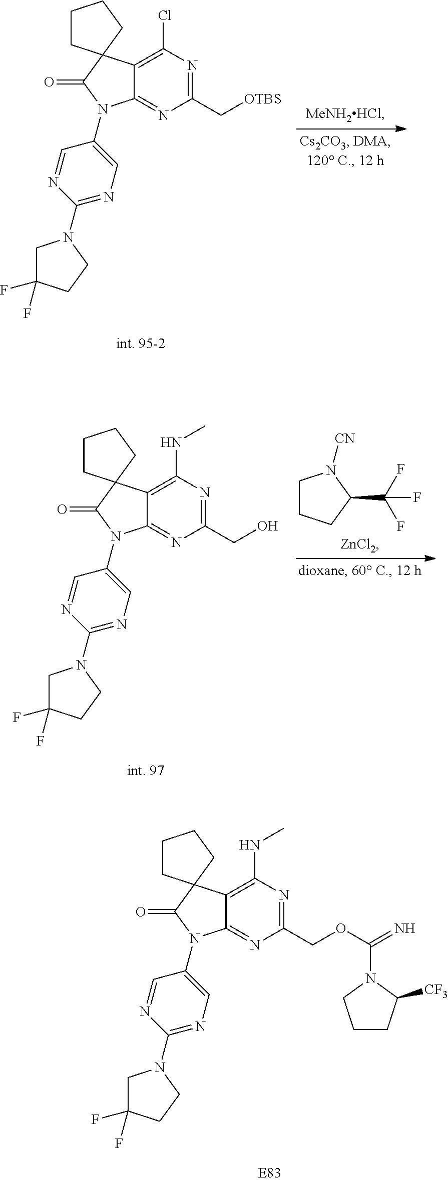

Compound E83 was synthesized as compound E15. ¹H NMR (400 MHz, CDCl₃) δ 8.49 (s, 2H), 5.21-5.03 (m, 2H), 4.96-4.44 (m, 1H), 4.32-4.28 (m, 1H), 3.95 (t, J=13.2 Hz, 2H), 3.84 (t, J=7.6 Hz, 2H), 3.54-3.42 (m, 2H), 3.08 (d, J=4.8 Hz, 3H), 2.54-2.43 (m, 2H), 2.54-1.87 (m, 12H). LCMS (ESI): m/z, 596.6 [M+H]⁺.

### Example 161. Preparation of Compound E84

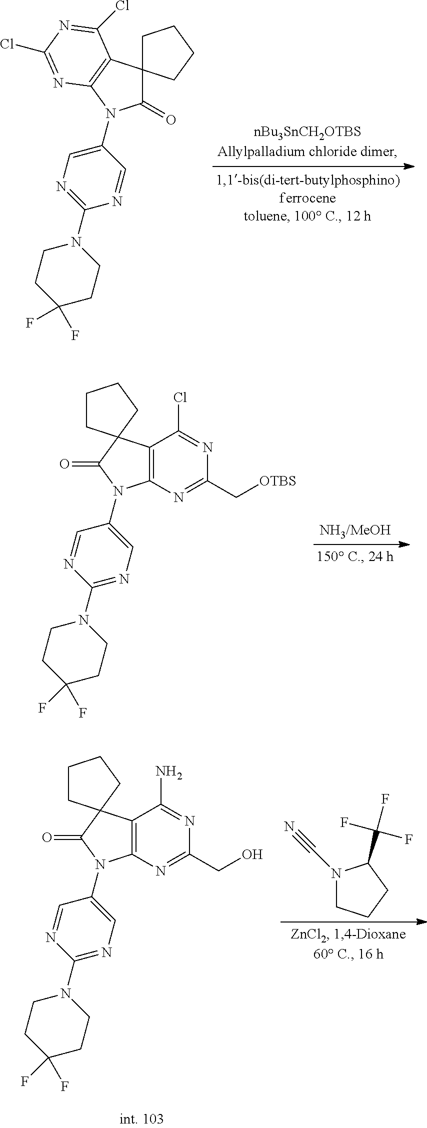 [SMILES: O=C1N(c2cnc(N3CCC(F)(F)CC3)nc2)c2nc(Cl)nc(Cl)c2C12CCCC2 ; CC(C)(C)[Si](C)(C)OCc1nc(Cl)c2c(n1)N(c1cnc(N3CCC(F)(F)CC3)nc1)C(=O)C21CCCC1 ; "NH3/MeOH" ; Nc1nc(CO)nc2c1C1(CCCC1)C(=O)N2c1cnc(N2CCC(F)(F)CC2)nc1 ; N#CN1CCC[C@@H]1C(F)(F)F]

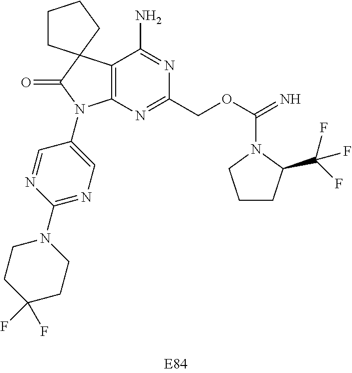 [SMILES: N=C(OCc1nc(N)c2c(n1)N(c1cnc(N3CCC(F)(F)CC3)nc1)C(=O)C21CCCC1)N1CCC[C@@H]1C(F)(F)F]

Compound E46 was synthesized as compound E45. ¹H NMR (400 MHz, CDCl₃) δ 8.48 (s, 2H), 5.30-4.99 (m, 2H), 4.79 (s, 2H), 4.45 (t, J=7.6 Hz, 1H), 4.09-3.86 (m, 4H), 3.51-3.43 (m, 2H), 2.31-1.81 (m, 16H). LCMS (ESI): m/z, 596.4 [M+H]⁺.

### Example 162. Preparation of Compound E85

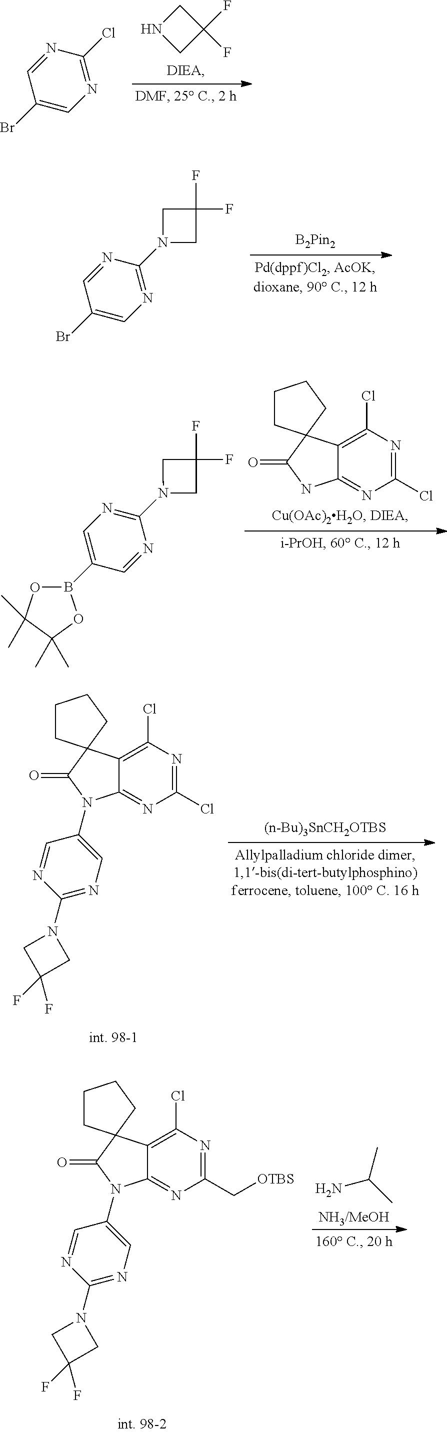

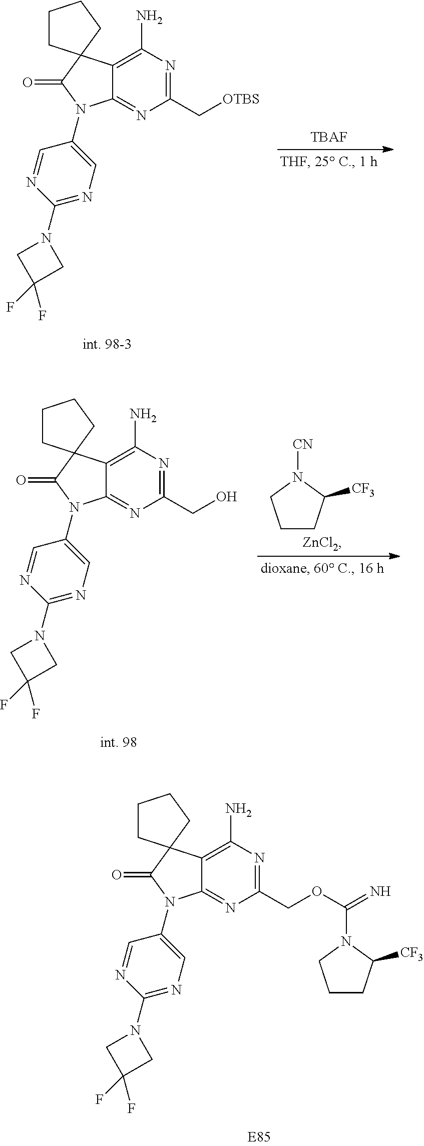

Compound E85 was synthesized as compound E60. LCMS (ESI): m/z, 568.5 [M+H]; ¹H NMR (400 MHz, DMSO-d₆) δ 8.52 (s, 2H), 6.62 (s, 2H), 5.77 (s, 1H), 5.00-4.85 (m, 2H), 4.54-4.48 (m, 5H), 3.35-3.00 (m, 2H), 2.16-2.13 (m, 2H), 1.96-1.83 (m, 10H).

### Example 163. Preparation of Compound E86

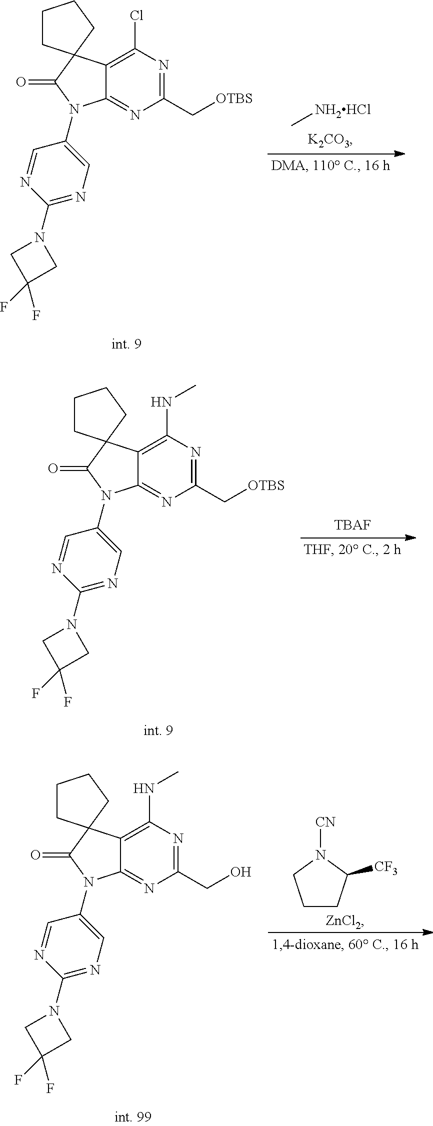

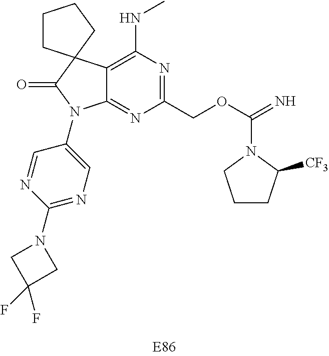 [SMILES: CNc1nc(COC(=N)N2CCC[C@@H]2C(F)(F)F)nc2c1C1(CCCC1)C(=O)N2c1cnc(N2CC(F)(F)C2)nc1]

Compound E86 was synthesized as compound E22. ¹H NMR (400 MHz, CDCl₃) (δ 8.56 (d, J=0.8 Hz, 2H), 5.20 (d, J=14.8 Hz, 1H), 5.07 (d, J=14.8 Hz, 1H), 4.49 (t, J=12.4 Hz, 5H), 4.31 (s, 1H), 3.52 (s, 2H), 3.09 (d, J=4.8 Hz, 3H), 2.19-2.07 (m, 6H), 2.05-1.87 (m, 6H). LCMS (ESI): m/z, 582.5 [M+H]⁺.

### Example 164. Preparation of Compound E87

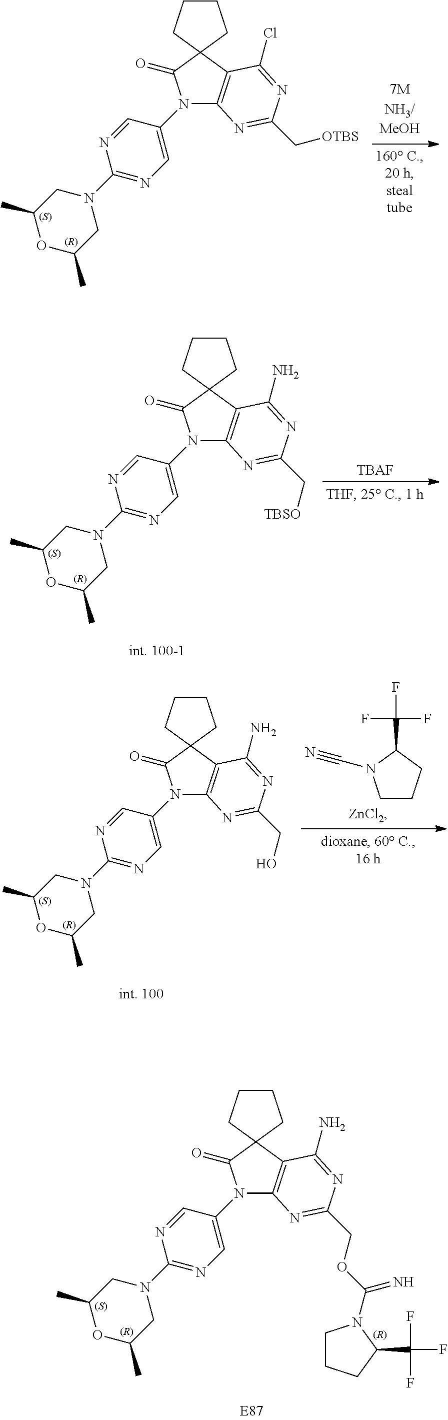

1. Synthesis of compound int. 100-1. To a solution of 2′-(((tert-butyldimethylsilyl)oxy)methyl)-4′-chloro-7′-(2-((2S,6R)-2,6-dimethylmorpholino)pyrimidin-5-yl)spiro[cyclopentane-1,5′-pyrrolo[2,3-d]pyrimidin]-6′(7′H)-one (3.00 g, 5.36 mmol) in NH₃ (100 mL, 7 M in MeOH) was stirred at 160° C. for 20 h in seal tube. The solution was concentrated under vacuum to afford 4′-amino-2′-(((tert-butyldimethylsilyl)oxy)methyl)-7′-(2-((2S,6R)-2,6-dimethylmorpholino)pyrimidin-5-yl)spiro[cyclopentane-1,5′-pyrrolo[2,3-d]pyrimidin]-6′(7′H)-one (3.0 g crude).

2. Synthesis of compound int. 100. A mixture of 4′-amino-2′-(((tert-butyldimethylsilyl)oxy)methyl)-7′-(2-((2S,6R)-2,6-dimethylmorpholino)pyrimidin-5-yl)spiro[cyclopentane-1,5′-pyrrolo[2,3-d]pyrimidin]-6′(7′H)-one (3.00 g, 5.56 mmol) and TBAF (16.7 mmol, 1 M in THF) in THF (30 mL) was stirred at 25° C. for 1 h. To the mixture was added H₂O, and the aqueous phase was extracted with EA. The combined organic layers were washed with brine and dried over Na₂SO₄, filtered and concentrated. The residue was purified by column chromatography to afford 4′-amino-7′-(2-((2S,6R)-2,6-dimethylmorpholino)pyrimidin-5-yl)-2′-(hydroxymethyl)spiro[cyclopentane-1,5′-pyrrolo[2,3-d]pyrimidin]-6′(7′H)-one (460 mg). LCMS (ESI): m/z, 426.3 [M+H]⁺.

3. Synthesis of compound E87. A mixture of (2R)-2-(trifluoromethyl)pyrrolidine-1-carbonitrile (533 mg, 3.24 mmol), 4,4′-amino-7′-(2-((2S,6R)-2,6-dimethylmorpholino)pyrimidin-5-yl)-2′-(hydroxymethyl)spiro[cyclopentane-1,5′-pyrrolo[2,3-d]pyrimidin]-6′(7′H)-one (460 mg, 1.08 mmol) and ZnCl₂ (295 mg, 2.16 mmol) in 1,4-dioxane (10 mL) was stirred at 60° C. for 16 h under N₂ atmosphere. The reaction mixture was concentrated. The residue was purified by Prep-HPLC to afford (4′-amino-7′-(2-((2S,6R)-2,6-dimethylmorpholino)pyrimidin-5-yl)-6′-oxo-6′,7′-dihydrospiro[cyclopentane-1,5′-pyrrolo[2,3-d]pyrimidin]-2′-yl)methyl (R)-2-(trifluoromethyl)pyrrolidine-1-carbimidate (294 mg). LCMS (ESI): m/z, 590.6 [M+H]⁺; ¹H NMR (400 MHz, DMSO-d₆) δ 8.41 (s, 2H), 6.55-6.17 (m, 2H), 5.87-5.77 (m, 1H), 5.01-4.86 (m, 2H), 4.53-4.50 (m, 3H), 3.58-3.52 (m, 2H), 3.32-3.24 (m, 2H), 2.59-2.49 (m, 2H), 2.15-2.07 (m, 2H), 1.98-1.86 (m, 10H), 1.16 (d, J=6.0 Hz, 6H).

### Example 165. Preparation of Compound E88

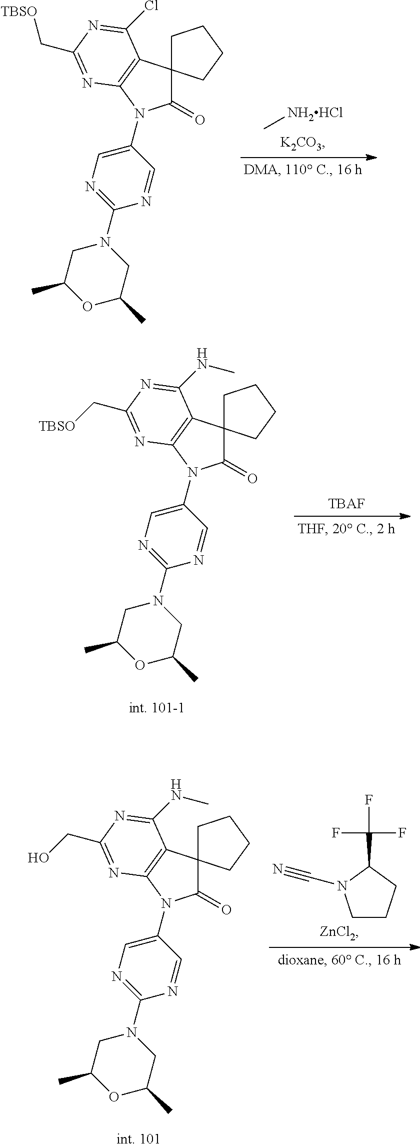

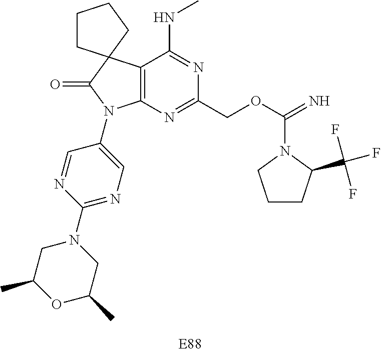 [SMILES: CNc1nc(COC(=N)N2CCC[C@@H]2C(F)(F)F)nc2c1C1(CCCC1)C(=O)N2c1cnc(N2C[C@@H](C)O[C@@H](C)C2)nc1]

1. Synthesis of compound int. 101-1. To a solution of 2′-{[(tert-butyldimethylsilyl)oxy]methyl}-4′-chloro-7′-{2-[(2R,6S)-2,6-dimethylmorpholin-4-yl]pyrimidin-5-yl}-6′,7′-dihydrospiro[cyclopentane-1,5′-pyrrolo[2,3-d]pyrimidin]-6′-one (300 mg, 0.54 mmol) in DMA (3 mL) were added K₂CO₃ (371 mg, 2.68 mmol) and MeNH₂·HCl (181 mg, 2.68 mmol). The mixture was stirred at 110° C. for 16 h. Water (20 mL) was added, and the mixture was extracted with EtOAc. The combined organic phases were washed with brine, dried over anhydrous Na₂SO₄, filtered and concentrated to give 2′-{[(tert-butyldimethylsilyl)oxy]methyl}-7′-{2-[(2R,6S)-2,6-dimethylmorpholin-4-yl]pyrimidin-5-yl}-4′-(methylamino)-6′,7′-dihydrospiro[cyclopentane-1,5′-pyrrolo[2,3-d]pyrimidin]-6′-one (297 mg crude).

2. Synthesis of compound int. 101. To a solution of 2′-{[(tert-butyldimethylsilyl)oxy]methyl}-7′-{2-[(2R,6S)-2,6-dimethylmorpholin-4-yl]pyrimidin-5-yl}-4′-(methylamino)-6′,7′-dihydrospiro[cyclopentane-1,5′-pyrrolo[2,3-d]pyrimidin]-6′-one (297 mg, 0.54 mmol) in THF (5 mL) was added TBAF (1.07 mL, 1 M in THF). The mixture was stirred at 20° C. for 2 h. Water (20 mL) was added, and the mixture was extracted with EtOAc. The combined organic phases were washed with brine, dried over anhydrous Na₂SO₄, filtered and concentrated. The residue was purified by Prep-TLC to give 7′-{2-[(2R,6S)-2,6-dimethylmorpholin-4-yl]pyrimidin-5-yl}-2′-(hydroxymethyl)-4′-(methylamino)-6′,7′-dihydrospiro[cyclopentane-1,5′-pyrrolo[2,3-d]pyrimidin]-6′-one (150 mg). LCMS (ESI): m/z, 418.2 [M+H]⁺.

3. Synthesis of compound E88. To a solution of 7′-{2-[(2R,6S)-2,6-dimethylmorpholin-4-yl]pyrimidin-5-yl}-2′-(hydroxymethyl)-4′-(methylamino)-6′,7′-dihydrospiro[cyclopentane-1,5′-pyrrolo[2,3-d]pyrimidin]-6′-one (480 mg, 1.09 mmol) and (2R)-2-(trifluoromethyl)pyrrolidine-1-carbonitrile (538 mg, 3.28 mmol) in 1,4-dioxane (10 mL) was added ZnCl₂ (447 mg, 3.28 mmol). The mixture was stirred at 60° C. for 16 h under N₂ atmosphere. The mixture was filtered and the filtrate was concentrated. The residue was purified by Prep-HPLC to give (7′-{2-[(2R,6S)-2,6-dimethylmorpholin-4-yl]pyrimidin-5-yl}-4′-(methylamino)-6′-oxo-6′,7′-dihydrospiro[cyclopentane-1,5′-pyrrolo[2,3-d]pyrimidin]-2′-yl)methyl (2R)-2-(trifluoromethyl)pyrrolidine-1-carboximidate (338 mg). ¹H NMR (400 MHz, CDCl₃) δ 8.45 (s, 2H), 5.21 (d, J=14.8 Hz, 1H), 5.06 (dd, J=2.4, 14.8 Hz, 1H), 4.63-4.54 (m, 2H), 4.50 (d, J=7.2 Hz, 1H), 4.29 (s, 1H), 4.05-4.02 (m, 1H), 3.66-3.61 (m, 2H), 3.50 (q, J=8.0, 9.2 Hz, 2H), 3.08 (dd, J=1.2, 4.8 Hz, 3H), 2.62 (dd, J=10.6, 13.2 Hz, 2H), 2.17-2.12 (m, 6H), 2.05-1.96 (m, 6H), 1.26 (d, J=6.2 Hz, 6H). LCMS (ESI): m/z, 604.6 [M+H]⁺.

### Example 166. Preparation of Compound E89

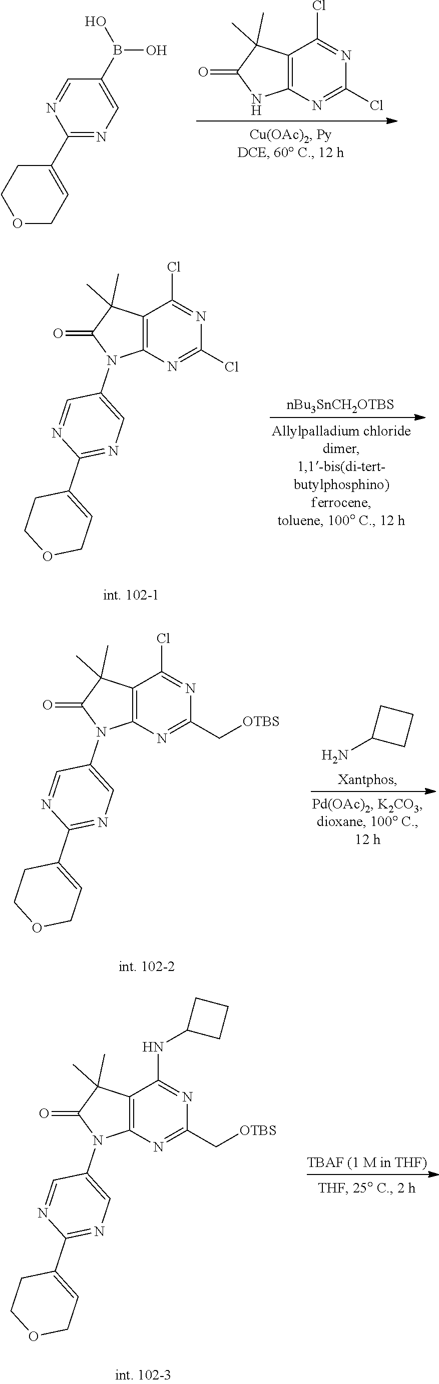

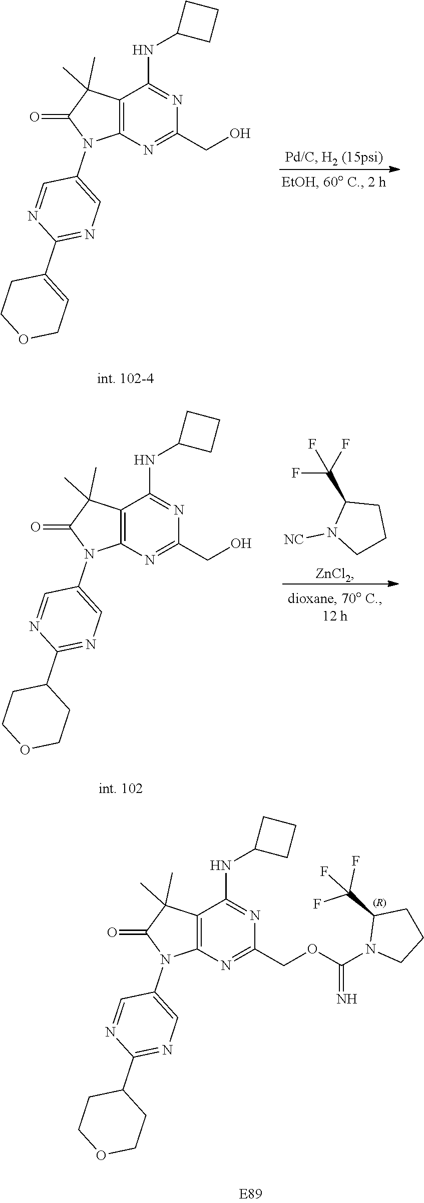

1. Synthesis of compound int. 102-4. Compound int. 102-4 was synthesized as compound int. 64. LCMS (ESI): m/z, 423.3 [M+H]⁺.

2. Synthesis of compound int. 102. To a solution of 4-(cyclobutylamino)-7-(2-(3,6-dihydro-2H-pyran-4-yl)pyrimidin-5-yl)-2-(hydroxymethyl)-5,5-dimethyl-5,7-dihydro-6H-pyrrolo[2,3-d]pyrimidin-6-one (300 mg, 0.710 mmol) in EtOH (10 mL) was added Pd/C (151 mg, 10%, 0.142 mmol). The mixture was stirred at 60° C. for 2 h under H₂ atmosphere. The mixture was filtered. The filtrate was concentrated to afford 4-(cyclobutylamino)-2-(hydroxymethyl)-5,5-dimethyl-7-(2-(tetrahydro-2H-pyran-4-yl)pyrimidin-5-yl)-5,7-dihydro-6H-pyrrolo[2,3-d]pyrimidin-6-one (120 mg). LCMS (ESI): m/z, 425.2 [M+H]⁺.

3. Synthesis of compound E89. Compound E89 was synthesized as compound E16. ¹H NMR (400 MHz, CDCl₃) (9.00 (s, 2H), 5.22-5.06 (m, 2H), 4.74-4.65 (m, 1H), 4.52-4.44 (m, 2H), 4.12-4.08 (m, 2H), 3.60-3.38 (m, 4H), 3.20-3.12 (m, 1H), 2.47-2.41 (m, 2H), 2.16-1.91 (m, 10H), 1.82-1.77 (m, 2H), 1.52 (s, 6H). LCMS (ESI): m/z, 589.7 [M+H]⁺.

### Example 167. Preparation of Compound E90

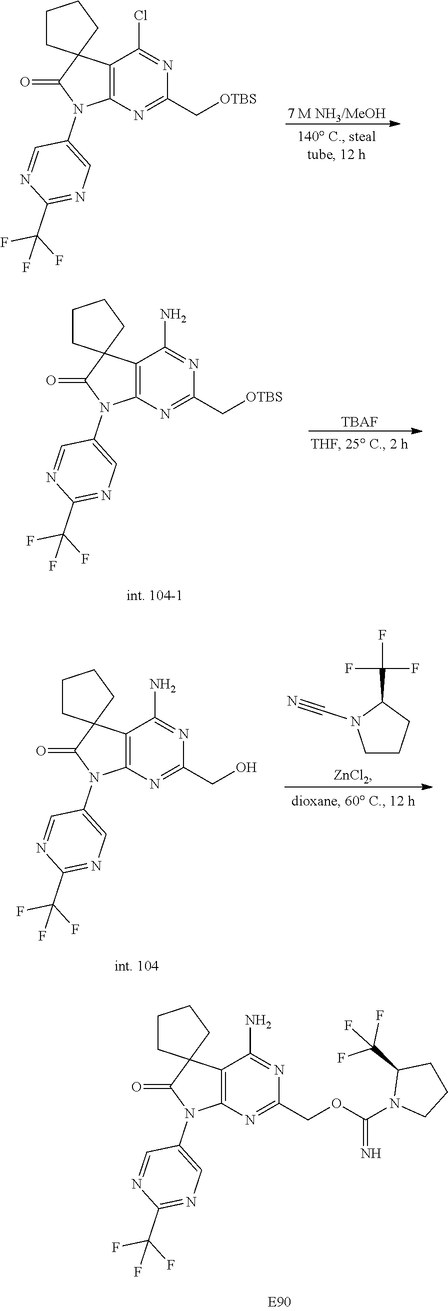 [SMILES: CC(C)(C)[Si](C)(C)OCc1nc(Cl)c2c(n1)N(c1cnc(C(F)(F)F)nc1)C(=O)C21CCCC1 ; CC(C)(C)[Si](C)(C)OCc1nc(N)c2c(n1)N(c1cnc(C(F)(F)F)nc1)C(=O)C21CCCC1 ; "TBAF" ; Nc1nc(CO)nc2c1C1(CCCC1)C(=O)N2c1cnc(C(F)(F)F)nc1 ; N#CN1CCC[C@@H]1C(F)(F)F ; N=C(OCc1nc(N)c2c(n1)N(c1cnc(C(F)(F)F)nc1)C(=O)C21CCCC1)N1CCC[C@@H]1C(F)(F)F]

Compound E90 was synthesized as compound E37. ¹H NMR (400 MHz, CDCl₃) δ 9.32 (s, 2H), 6.29 (s, 2H), 5.34-5.23 (m, 2H), 4.57 (s, 1H), 3.86-3.76 (m, 2H), 2.30-2.04 (m, 12H). LCMS (ESI): m/z, 545.5 [M+H]⁺.

### Example 168. Preparation of Compound E91

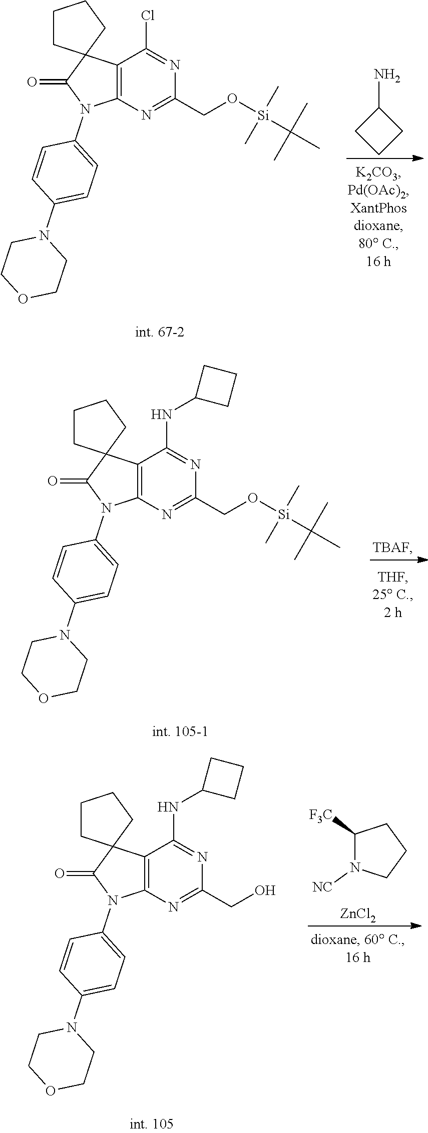

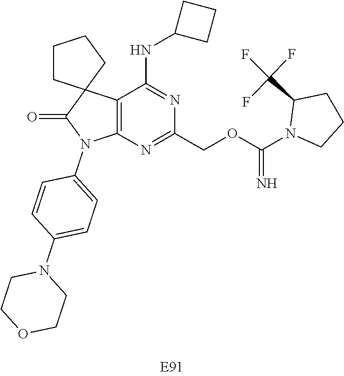 [SMILES: N=C(OCc1nc(NC2CCC2)c2c(n1)N(c1ccc(N3CCOCC3)cc1)C(=O)C21CCCC1)N1CCC[C@@H]1C(F)(F)F]

Compound E91 was synthesized as compound E21. LCMS (ESI): m/z, 614.7 [M+H]⁺; ¹H NMR (400 MHz, DMSO-d₆) δ 7.20-7.17 (m, 2H), 7.00-6.98 (m, 2H), 6.20-6.14 (m, 1H), 5.76-5.70 (m, 1H), 5.00-4.85 (m, 2H), 4.72-4.62 (m, 1H), 4.57-4.53 (m, 1H), 3.76-3.73 (m, 4H), 3.15-3.12 (m, 4H), 2.21-2.09 (m, 6H), 2.07-1.80 (m, 10H), 1.83-1.80 (m, 2H).

### Example 169. Preparation of Compound E92

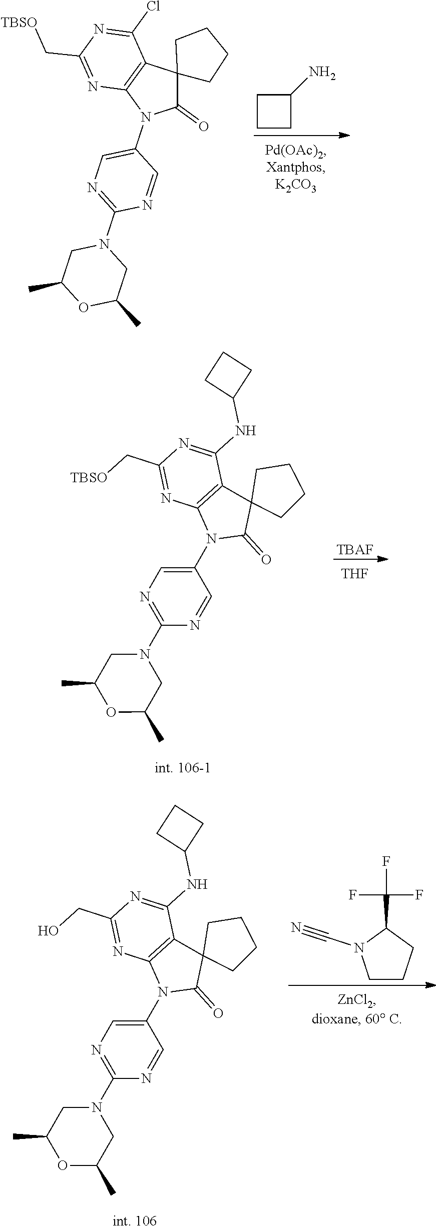

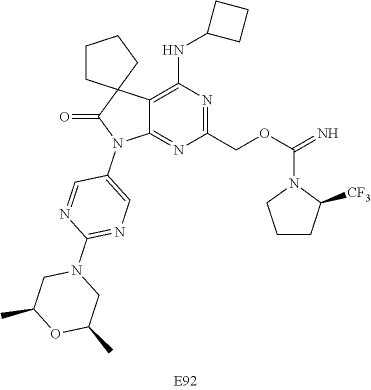 [SMILES: C[C@@H]1CN(c2ncc(N3C(=O)C4(CCCC4)c4c(NC5CCC5)nc(COC(=N)N5CCC[C@@H]5C(F)(F)F)nc43)cn2)C[C@H](C)O1]

1. Synthesis of compound int. 106-1. To a solution of 2′-{[(tert-butyldimethylsilyl)oxy]methyl}-4′-chloro-7′-{2-[(2R,6S)-2,6-dimethylmorpholin-4-yl]pyrimidin-5-yl}-6′,7′-dihydrospiro[cyclopentane-1,5′-pyrrolo[2,3-d]pyrimidin]-6′-one (300 mg, 0.54 mmol) and cyclobutylamine (0.14 mL) in 1,4-dioxane (12 mL) were added Pd(OAc)₂ (12 mg, 0.054 mmol), K₂CO₃ (148 mg, 1.07 mmol) and XantPhos (31 mg, 0.054 mmol). The mixture was stirred at 100° C. for 16 h under N₂ atmosphere. The mixture was filtered and the filtrate was concentrated to give 2′-{[(tert-butyldimethylsilyl)oxy]methyl}-4′-(cyclobutylamino)-7′-{2-[(2R,6S)-2,6-dimethylmorpholin-4-yl]pyrimidin-5-yl}-6′,7′-dihydrospiro[cyclopentane-1,5′-pyrrolo[2,3-d]pyrimidin]-6′-one (300 mg crude).

2. Synthesis of compound int. 106. To a solution of 2′-{[(tert-butyldimethylsilyl)oxy]methyl}-4′-(cyclobutylamino)-7′-{2-[(2R,6S)-2,6-dimethylmorpholin-4-yl]pyrimidin-5-yl}-6′,7′-dihydrospiro[cyclopentane-1,5′-pyrrolo[2,3-d]pyrimidin]-6′-one (300 mg, 0.51 mmol) in THF (5 mL) was added TBAF (1.52 mL, 1 M in THF) and stirred at 20° C. for 2 h. Water (20 mL) was added and the mixture was extracted with EtOAc. The combined organic phases were washed with brine, dried over anhydrous Na₂SO₄, filtered and concentrated. The residue was purified by Prep-TLC to give 4′-(cyclobutylamino)-7′-{2-[(2R,6S)-2,6-dimethylmorpholin-4-yl]pyrimidin-5-yl}-2′-(hydroxymethyl)-6′,7′-dihydrospiro[cyclopentane-1,5′-pyrrolo[2,3-d]pyrimidin]-6′-one (50 mg). LCMS (ESI): t_{R}=2.518, m/z, 480.2 [M+H]⁺.

3. Synthesis of compound E92. To a solution of 4′-(cyclobutylamino)-7′-{2-[(2R,6S)-2,6-dimethylmorpholin-4-yl]pyrimidin-5-yl}-2′-(hydroxymethyl)-6′,7′-dihydrospiro[cyclopentane-1,5′-pyrrolo[2,3-d]pyrimidin]-6′-one (77 mg, 0.1 mmol) and (2R)-2-(trifluoromethyl)pyrrolidine-1-carbonitrile (51 mg, 0.31 mmol) in 1,4-dioxane (3 mL) was added ZnCl₂ (43 mg, 0.31 mmol). The mixture was stirred at 60° C. for 16 h under N₂ atmosphere. The mixture was filtered. The filtrate was purified by Prep-HPLC to give [4′-(cyclobutylamino)-7′-{2-[(2R,6S)-2,6-dimethylmorpholin-4-yl]pyrimidin-5-yl}-6′-oxo-6′,7′-dihydrospiro[cyclopentane-1,5′-pyrrolo[2,3-d]pyrimidin]-2′-yl]methyl (2R)-2-(trifluoromethyl)pyrrolidine-1-carboximidate (15 mg). ¹H NMR (400 MHz, CDCl3) (8.45 (s, 2H), 5.17 (d, J=14.8 Hz, 1H), 5.02 (d, J=14.8 Hz, 1H), 4.67 (q, J=7.6 Hz, 1H), 4.58 (dd, J=2.4, 13.6 Hz, 2H), 4.52-4.43 (m, 1H), 4.39 (d, J=7.6 Hz, 1H), 3.65-3.60 (m, 2H), 3.48-3.40 (m, 2H), 2.65-2.59 (m, 2H), 2.48-2.38 (m, 2H), 2.22-1.99 (m, 8H), 1.97-1.71 (m, 8H), 1.26 (d, J=6.0 Hz, 6H). LCMS (ESI): m/z, 644.7 [M+H]⁺.

### Example 170. Preparation of Compound E93

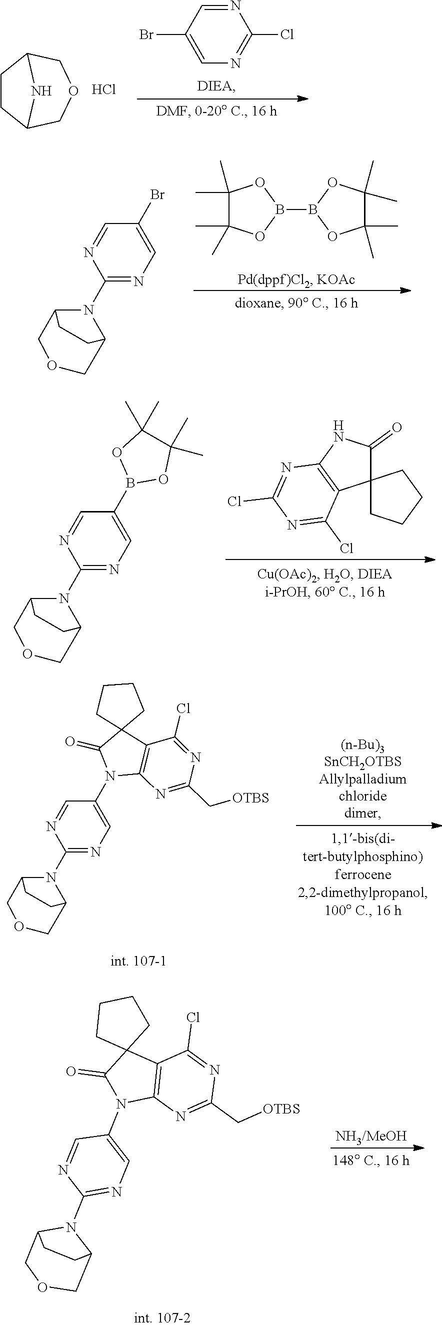

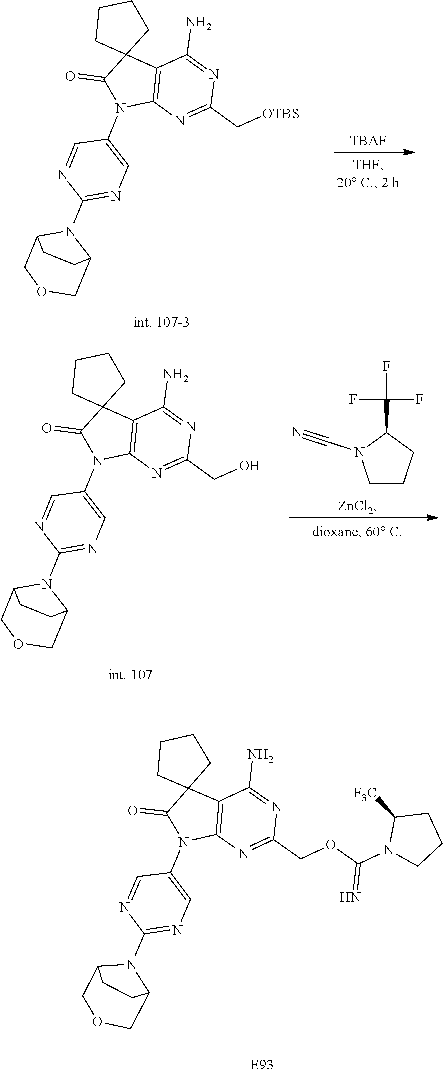 [SMILES: CC(C)(C)[Si](C)(C)OCc1nc(N)c2c(n1)N(c1cnc(N3C4CCC3COC4)nc1)C(=O)C21CCCC1 ; "TBAF" ; Nc1nc(CO)nc2c1C1(CCCC1)C(=O)N2c1cnc(N2C3CCC2COC3)nc1 ; N#CN1CCC[C@@H]1C(F)(F)F ; N=C(OCc1nc(N)c2c(n1)N(c1cnc(N3C4CCC3COC4)nc1)C(=O)C21CCCC1)N1CCC[C@@H]1C(F)(F)F]

1. Synthesis of compound 8-(5-bromopyrimidin-2-yl)-3-oxa-8-azabicyclo[3.2.1]octane. To a solution of 3-Oxa-8-azabicyclo[3.2.1]octane, hydrochloride (5.0 g, 33.4 mmol) and 5-bromo-2-chloropyrimidine (6.46 g, 33.4 mmol) in DMF (60 mL) was added DIPEA (16.6 mL) at 0° C. and stirred at 20° C. for 16 h. Water (100 mL) was added and the white solid was precipitated. The solid was filtered and dried to give 8-(5-bromopyrimidin-2-yl)-3-oxa-8-azabicyclo[3.2.1]octane (5.0 g).

2. Synthesis of compound 8-[5-(4,4,5,5-tetramethyl-1,3,2-dioxaborolan-2-yl)pyrimidin-2-yl]-3-oxa-8-azabicyclo[3.2.1]octane. To a solution of 8-(5-bromopyrimidin-2-yl)-3-oxa-8-azabicyclo[3.2.1]octane (4.80 g, 17.8 mmol) and Pin₂B₂ (6.77 g, 26.6 mmol) in 1,4-dioxane (100 mL) were added KOAc (3.49 g, 35.5 mmol) and Pd(dppf)₂Cl₂ (1.30 g, 1.78 mmol). The mixture was stirred at 90° C. for 16 h under N₂ atmosphere. The solvent was removed and the residue was treated with DCM (100 mL). The mixture was filtered, and the filtrate was concentrated. The residue was purified by column chromatography to give 8-[5-(4,4,5,5-tetramethyl-1,3,2-dioxaborolan-2-yl)pyrimidin-2-yl]-3-oxa-8-azabicyclo[3.2.1]octane (5.0 g).

3. Synthesis of compound int. 107-1. To a solution of 2′,4′-dichloro-6′,7′-dihydrospiro[cyclopentane-1,5′-pyrrolo[2,3-d]pyrimidin]-6′-one (2.70 g, 10.5 mmol) and 8-[5-(4,4,5,5-tetramethyl-1,3,2-dioxaborolan-2-yl)pyrimidin-2-yl]-3-oxa-8-azabicyclo[3.2.1]octane (4.98 g, 15.7 mmol) in i-PrOH (50 mL) were added Cu(OAc)₂·H₂O (2.51 g, 12.5 mmol), DIPEA (5.19 mL) and 4A Molecular Sieve (4.61 g). The mixture was stirred at 60° C. for 20 h under O₂ (15 psi) atmosphere. The solvent was removed, and the residue was treated with DCM (50 mL). The mixture was filtered and the filtrate was concentrated. The residue was purified by column chromatography to give title compound (4.0 g). LCMS (ESI): m/z, 447.4 [M+H]⁺.

4. Synthesis of compound int. 107-2. To a solution of 2′,4′-dichloro-7′-(2-{3-oxa-8-azabicyclo[3.2.1]octan-8-yl}pyrimidin-5-yl)-6′,7′-dihydrospiro[cyclopentane-1,5′-pyrrolo[2,3-d]pyrimidin]-6′-one (4.30 g, 9.61 mmol) and (tert-butyldimethylsilyloxymethyl)tri-n-butyltin (4.39 g, 10.1 mmol) in 2,2-dimethylpropanol (50 mL) were added [PdCl(allyl)]₂ (141 mg, 0.38 mmol) and 1,1′-bis(di-tert-butylphosphino)ferrocene (365 mg, 0.77 mmol). The mixture was stirred at 100° C. for 16 hours under N₂ atmosphere. The solvent was removed. The residue was purified by column chromatography to give title compound (1.80 g). LCMS (ESI): m/z, 577.6 [M+H]⁺.

5. Synthesis of compound int. 107-3. A solution of 2′-{[(tert-butyldimethylsilyl)oxy]methyl}-4′-chloro-7′-(2-{3-oxa-8-azabicyclo[3.2.1]octan-8-yl}pyrimidin-5-yl)-6′,7′-dihydrospiro[cyclopentane-1,5′-pyrrolo[2,3-d]pyrimidin]-6′-one (100 mg, 0.18 mmol) in NH₃ (20 mL, 7 M in MeOH) was stirred at 148° C. for 16 h. The mixture was concentrated to yield title compound (96.5 mg crude).

6. Synthesis of compound int. 107. To a solution of 4′-amino-2′-{[(tert-butyldimethylsilyl)oxy]methyl}-7′-(2-{3-oxa-8-azabicyclo[3.2.1]octan-8-yl}pyrimidin-5-yl)-6′,7′-dihydrospiro[cyclopentane-1,5′-pyrrolo[2,3-d]pyrimidin]-6′-one (96 mg, 0.18 mmol) in THF (3 mL) was added TBAF (0.36 mL, 1 M in THF). The mixture was stirred at 20° C. for 2 hours. Water (20 mL) was added and the mixture was extracted with EtOAc. The combined organic phases were washed with brine, dried over anhydrous Na₂SO₄, filtered and concentrated. The residue was purified by Prep-TLC to give title compound (50 mg). LCMS (ESI): m/z, 424.5 [M+H]⁺.

7. Synthesis of compound E93. To a solution of 4′-amino-2′-(hydroxymethyl)-7′-(2-{3-oxa-8-azabicyclo[3.2.1]octan-8-yl}pyrimidin-5-yl)-6′,7′-dihydrospiro[cyclopentane-1,5′-pyrrolo[2,3-d]pyrimidin]-6′-one (55 mg, 0.12 mmol) and (2R)-2-(trifluoromethyl)pyrrolidine-1-carbonitrile (39 mg, 0.24 mmol) in 1,4-dioxane (3 mL) was added ZnCl₂ (32 mg, 0.24 mmol). The mixture was stirred at 60° C. for 16 hours under N₂ atmosphere. The solvent was removed. The residue was purified by Prep-HPLC to yield title compound (17.7 mg). ¹H NMR (400 MHz, CDCl₃) δ 8.38 (s, 2H), 5.24-4.90 (m, 4H), 4.63 (s, 2H), 4.48 (s, 1H), 3.76-3.57 (m, 4H), 3.50 (s, 2H), 2.18-1.82 (m, 16H). LCMS (ESI): m/z, 588.7 [M+H]⁺.

### Example 171. Preparation of Compound E94

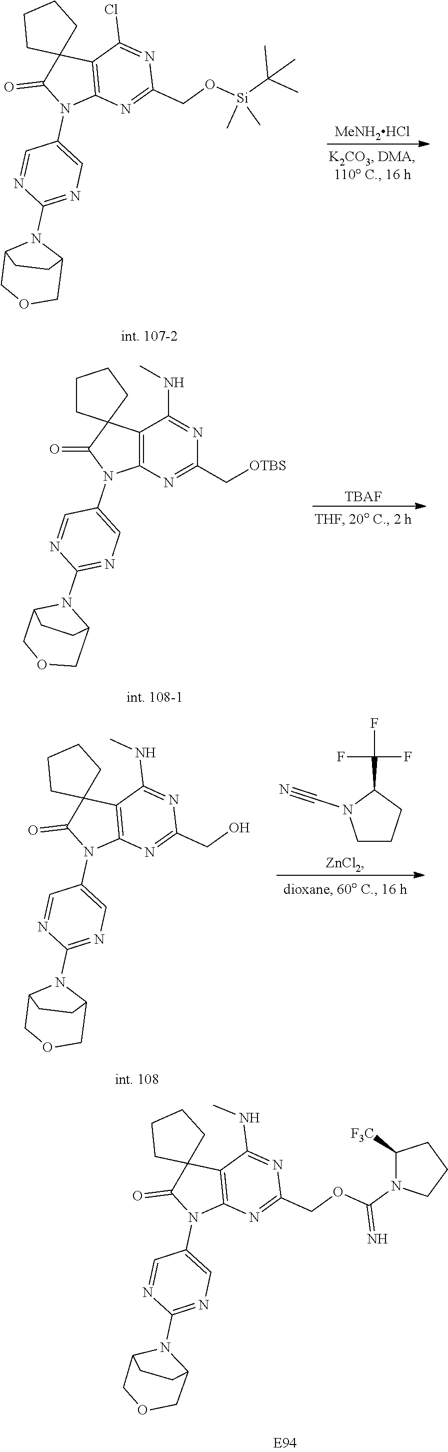

Compound E94 was synthesized as compound E22. ¹H NMR (400 MHz, CDCl₃) δ 8.46 (s, 2H), 5.28-5.03 (m, 2H), 4.76-4.65 (m, 2H), 4.49 (t, J=7.6 Hz, 1H), 4.30 (d, J=5.2 Hz, I1H), 3.83-3.62 (m, 4H), 3.50 (dd, J=8.4, 16.0 Hz, 2H), 3.09 (d, J=4.8 Hz, 3H), 2.27-2.04 (m, 9H), 2.03-1.84 (m, 7H). LCMS (ESI): m/z, 588.7 [M+H]⁺.

### Example 172. Preparation of Compound E95

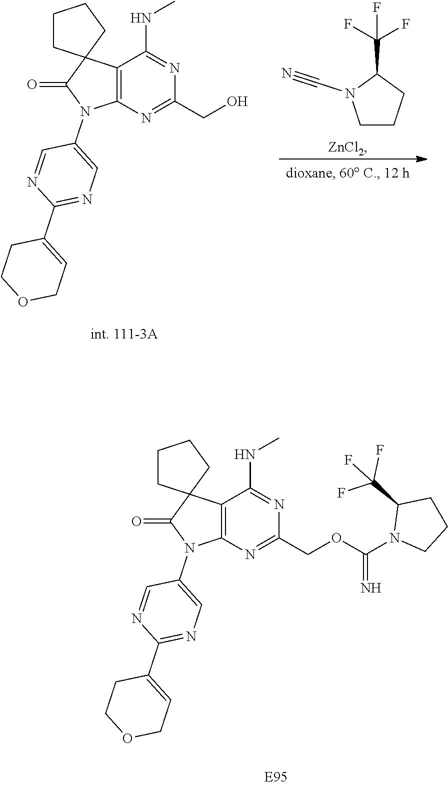 [SMILES: CNc1nc(CO)nc2c1C1(CCCC1)C(=O)N2c1cnc(C2=CCOCC2)nc1 ; N#CN1CCC[C@@H]1C(F)(F)F ; CNc1nc(COC(=N)N2CCC[C@@H]2C(F)(F)F)nc2c1C1(CCCC1)C(=O)N2c1cnc(C2=CCOCC2)nc1]

Compound E95 was synthesized as compound E16. ¹H NMR (400 MHz, CDCl₃) δ 9.02 (s, 2H), 6.57-6.55 (m, 0.6H), 5.26-5.21 (m, 1H), 5.12-5.08 (m, 1H), 5.02-4.99 (m, 0.6H), 4.50-4.46 (m, 1H), 4.35-4.33 (m, 1H), 4.21-4.17 (m, 0.6H), 4.13-4.08 (m, 0.6H), 3.96-3.93 (m, 1H), 3.84-3.82 (m, 0.6H), 3.56-3.48 (m, 2H), 3.09 (d, J=4.8 Hz, 3H), 2.74-2.71 (m, 1H), 2.31-2.27 (m, 1H), 2.17-1.91 (m, 14H). LCMS (ESI): m/z, 573.3 [M+H]⁺.

### Example 173. Preparation of Compound E96

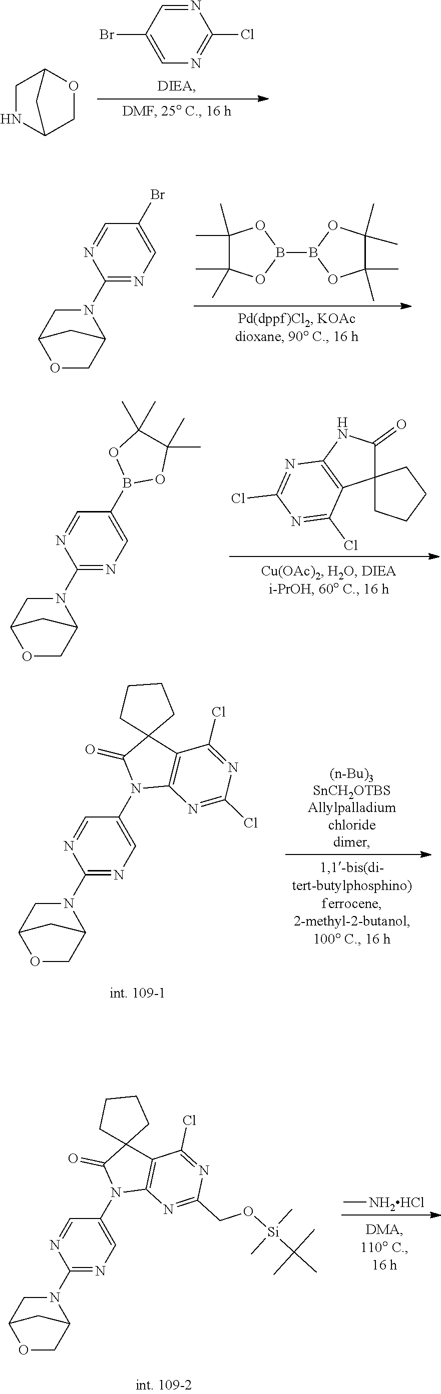

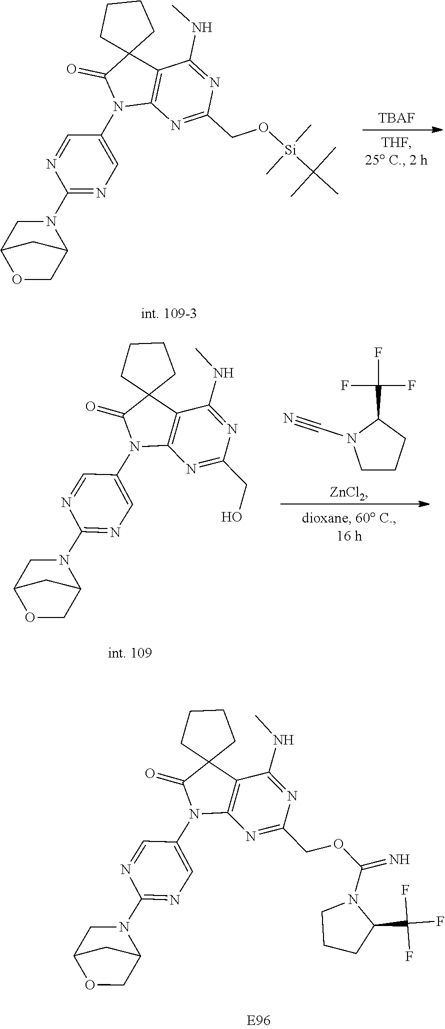 [SMILES: CNc1nc(CO[Si](C)(C)C(C)(C)C)nc2c1C1(CCCC1)C(=O)N2c1cnc(N2CC3CC2CO3)nc1 ; "TBAF" ; CNc1nc(CO)nc2c1C1(CCCC1)C(=O)N2c1cnc(N2CC3CC2CO3)nc1 ; N#CN1CCC[C@@H]1C(F)(F)F ; CNc1nc(COC(=N)N2CCC[C@@H]2C(F)(F)F)nc2c1C1(CCCC1)C(=O)N2c1cnc(N2CC3CC2CO3)nc1]

1. Synthesis of compound 5-(5-bromopyrimidin-2-yl)-2-oxa-5-azabicyclo[2.2.1]heptane. A mixture of 2-oxa-5-azabicyclo[2.2.1]heptane hydrochloride (5.00 g, 36.9 mmol), 5-bromo-2-chloropyrimidine (7.13 g, 36.9 mmol) and DIPEA (14.3 g, 111 mmol) in DMF (60 mL) was stirred at 25° C. for 2 h. To the mixture added H₂O (200 mL), the aqueous phase was extracted with EA. The combined organic layers were washed with brine and dried over Na₂SO₄, filtered and concentrated to afford 5-(5-bromopyrimidin-2-yl)-2-oxa-5-azabicyclo[2.2.1]heptane (9.0 g). ¹H NMR (400 MHz, DMSO-d₆) δ 8.43 (s, 2H), 4.89-4.81 (m, 1H), 4.67-4.64 (m, 1H), 3.78-3.76 (m, 1H), 3.65-3.63 (m, 1H), 3.45-3.42 (m, 1H), 3.35-3.28 (m, 1H), 1.94-1.83 (m, 2H).

2. Synthesis of compound 5-(5-(4,4,5,5-tetramethyl-1,3,2-dioxaborolan-2-yl)pyrimidin-2-yl)-2-oxa-5-azabicyclo[2.2.1]heptane. To a mixture of 5-(5-bromopyrimidin-2-yl)-2-oxa-5-azabicyclo[2.2.1]heptane (9.00 g, 35.1 mmol) in 1,4-dioxane (100 mL), Pin2B2 (13.4 g, 52.7 mmol), Pd(dppf)Cl₂ (1.29 g, 1.76 mmol), and KOAc (10.3 g, 105 mmol) were added in turn. The mixture was degassed under vacuum and purged with N₂ several times. The reaction mixture was stirred at 90° C. for 16 h under N₂ atmosphere. The reaction mixture was filtered, and the filtrate was concentrated. The residue was purified by column chromatography to afford 5-(5-(4,4,5,5-tetramethyl-1,3,2-dioxaborolan-2-yl)pyrimidin-2-yl)-2-oxa-5-azabicyclo[2.2.1]heptane (6.5 g). LCMS (ESI): m/z, 222.1 [M+H]⁺.

3. Synthesis of compound int. 109-1. A mixture of 2′,4′-dichloro-6′,7′-dihydrospiro[cyclopentane-1,5′-pyrrolo[2,3-d]pyrimidin]-6′-one (3.69 g, 14.3 mmol), 5-[5-(4,4,5,5-tetramethyl-1,3,2-dioxaborolan-2-yl)pyrimidin-2-yl]-2-oxa-5-azabicyclo[2.2.1]heptane (6.50 g, 21.4 mmol), Cu(OAc)₂·H₂O (3.14 g, 15.7 mmol), DIPEA (5.54 g, 42.9 mmol) and 4A Molecular Sieve (6.30 g) in i-PrOH (100 mL) was stirred at 60° C. for 12 hours under O₂ atmosphere. The reaction mixture was filtered and the filtrate was concentrated. The residue was purified by column chromatography to afford 7′-(2-(2-oxa-5-azabicyclo[2.2.1]heptan-5-yl)pyrimidin-5-yl)-2′,4′-dichlorospiro[cyclopentane-1,5′-pyrrolo[2,3-d]pyrimidin]-6′(7′H)-one (4.2 g). LCMS (ESI): m/z, 432.9 [M+H]⁺.

4. Synthesis of compound int. 109-2. A mixture of 7′-(2-(2-oxa-5-azabicyclo[2.2.1]heptan-5-yl)pyrimidin-5-yl)-2′,4′-dichlorospiro[cyclopentane-1,5′-pyrrolo[2,3-d]pyrimidin]-6′(7′H)-one (4.20 g, 9.69 mmol), (tert-butyldimethylsilyloxymethyl)tri-n-butyltin (5.06 g, 11.6 mmol), [PdCl(allyl)]₂ (178 mg, 0.48 mmol) and 1,1′-bis(di-tert-butylphosphino)ferrocene (460 mg, 0.97 mmol) in 2-methyl-2-butanol (80 mL) was stirred at 100° C. for 16 h under N₂ atmosphere. The reaction mixture was concentrated. The residue was purified by column chromatography to afford 7′-(2-(2-oxa-5-azabicyclo[2.2.1]heptan-5-yl)pyrimidin-5-yl)-2′-(((tert-butyldimethylsilyl)oxy)methyl)-4′-chlorospiro[cyclopentane-1,5′-pyrrolo[2,3-d]pyrimidin]-6′(7′H)-one (2.6 g). LCMS (ESI): m/z, 543.6 [M+H]⁺.

5. Synthesis of compound int. 109-3. A mixture of 7′-(2-(2-oxa-5-azabicyclo[2.2.1]heptan-5-yl)pyrimidin-5-yl)-2′-(((tert-butyldimethylsilyl)oxy)methyl)-4′-chlorospiro[cyclopentane-1,5′-pyrrolo[2,3-d]pyrimidin]-6′(7′H)-one (800 mg, 1.47 mmol), K₂CO₃ (1.02 g, 7.36 mmol) and methanamine, hydrochloride (498 mg, 7.36 mmol) in DMA (10 mL) was stirred at 110° C. for 16 h. To the mixture added H₂O (100 mL), the aqueous phase was extracted with EA. The combined organic layers were washed with brine and dried over Na₂SO₄, filtered and concentrated to afford 7′-(2-(2-oxa-5-azabicyclo[2.2.1]heptan-5-yl)pyrimidin-5-yl)-2′-(((tert-butyldimethylsilyl)oxy)methyl)-4′-(methylamino)spiro[cyclopentane-1,5′-pyrrolo[2,3-d]pyrimidin]-6′(7′H)-one (800 mg crude).

6. Synthesis of compound int. 109. To a solution of 7′-(2-(2-oxa-5-azabicyclo[2.2.1]heptan-5-yl)pyrimidin-5-yl)-2′-(((tert-butyldimethylsilyl)oxy)methyl)-4′-(methylamino)spiro[cyclopentane-1,5′-pyrrolo[2,3-d]pyrimidin]-6′(7′H)-one (792 mg, 1.47 mmol) in THF (20 mL) was added TBAF (4.42 mL, 1 M in THF). The reaction was stirred at 25° C. for 2 h. To the mixture added H₂O (50 mL), the aqueous phase was extracted with EA. The combined organic layers were washed with brine and dried over Na₂SO₄, filtered and concentrated. The residue was purified by column chromatography to afford 7′-(2-(2-oxa-5-azabicyclo[2.2.1]heptan-5-yl)pyrimidin-5-yl)-2′-(hydroxymethyl)-4′-(methylamino)spiro[cyclopentane-1,5′-pyrrolo[2,3-d]pyrimidin]-6′(7′H)-one (300 mg). LCMS (ESI): m/z, 424.2 [M+H]⁺.

7. Synthesis of compound E96. A mixture of (2R)-2-(trifluoromethyl)pyrrolidine-1-carbonitrile (349 mg, 2.13 mmol), 7′-(2-(2-oxa-5-azabicyclo[2.2.1]heptan-5-yl)pyrimidin-5-yl)-2′-(hydroxymethyl)-4′-(methylamino)spiro[cyclopentane-1,5′-pyrrolo[2,3-d]pyrimidin]-6′(7′H)-one (300 mg, 0.71 mmol) and ZnCl₂ (194 mg, 1.42 mmol) in 1,4-dioxane (5 mL) was stirred at 60° C. for 16 h under N₂ atmosphere. The reaction mixture was concentrated. The crude product was purified by Prep-HPLC to afford (7′-(2-(2-oxa-5-azabicyclo[2.2.1]heptan-5-yl)pyrimidin-5-yl)-4′-(methylamino)-6′-oxo-6′,7′-dihydrospiro[cyclopentane-1,5′-pyrrolo[2,3-d]pyrimidin]-2′-yl)methyl (2R)-2-(trifluoromethyl)pyrrolidine-1-carbimidate (226 mg). LCMS (ESI): m/z, 588.3 [M+H]⁺; ¹H NMR (400 MHz, DMSO-d₆) δ 8.38-8.36 (m, 2H), 6.32-6.31 (m, 1H), 5.77 (s, 1H), 5.03-4.91 (m, 3H), 4.71-4.67 (m, 1H), 4.56-4.54 (m, 1H), 3.83-3.80 (m, 1H), 3.68-3.67 (m, 1H), 3.52-3.48 (m, 1H), 3.41-3.39 (m, 1H), 3.35-3.33 (m, 2H), 2.89 (d, J=4.4 Hz, 3H), 2.14-2.00 (m, 2H), 1.98-1.85 (m, 12H).

### Example 174. Preparation of Compound E97

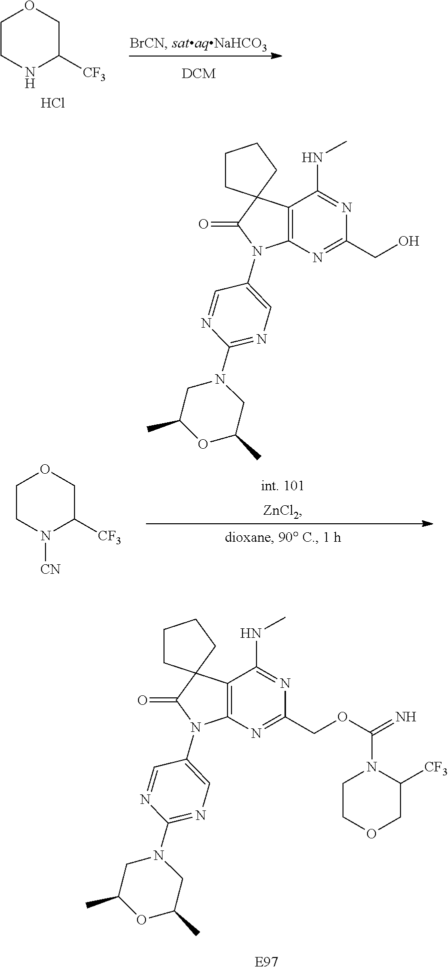 [SMILES: FC(F)(F)C1COCCN1 ; Cl ; CNc1nc(CO)nc2c1C1(CCCC1)C(=O)N2c1cnc(N2C[C@@H](C)O[C@@H](C)C2)nc1 ; N#CN1CCOCC1C(F)(F)F ; CNc1nc(COC(=N)N2CCOCC2C(F)(F)F)nc2c1C1(CCCC1)C(=O)N2c1cnc(N2C[C@@H](C)O[C@@H](C)C2)nc1]

1. Synthesis of compound 3-(trifluoromethyl)morpholine-4-carbonitrile. To a solution of 3-(Trifluoromethyl)morpholine hydrochloride (950 mg, 4.96 mmol) in DCM (10 mL) and sat. aq. NaHCO₃ (10 mL) was added BrCN (1.05 g, 9.92 mmol). The mixture was stirred at 20° C. for 16 h. Water (20 mL) was added, and the mixture was extracted with DCM. The combined organic phases were washed with brine, dried over anhydrous Na₂SO₄, filtered and concentrated. The residue was purified by column chromatography to give 3-(trifluoromethyl)morpholine-4-carbonitrile (850 mg).

2. Synthesis of compound E97. Compound E97 was synthesized as compound E16. ¹H NMR (400 MHz, CDCl₃) δ 8.33 (s, 2H), 5.03-4.81 (m, 2H), 4.54-4.40 (m, 3H), 4.21 (d, J=4.8 Hz, 1H), 4.03 (d, J=12.4 Hz, 1H), 3.80 (dd, J=3.6, 11.2 Hz, 1H), 3.72-3.37 (m, 5H), 3.34-3.21 (m, 1H), 2.97 (d, J=4.8 Hz, 3H), 2.55-2.49 (m, 2H), 2.11-1.99 (m, 4H), 1.93-1.88 (m, 2H), 1.84-1.74 (m, 2H), 1.14 (d, J=6.4 Hz, 6H). LCMS (ESI): m/z, 620.7 [M+H]⁺.

### Example 175. Preparation of Compound E98

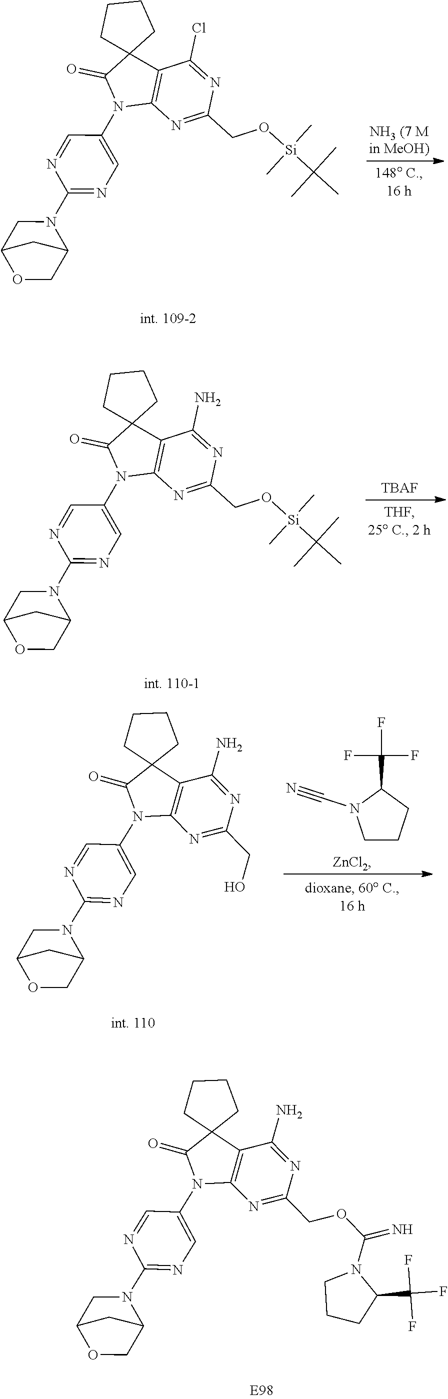 [SMILES: CC(C)(C)[Si](C)(C)OCc1nc(Cl)c2c(n1)N(c1cnc(N3CC4CC3CO4)nc1)C(=O)C21CCCC1 ; CC(C)(C)[Si](C)(C)OCc1nc(N)c2c(n1)N(c1cnc(N3CC4CC3CO4)nc1)C(=O)C21CCCC1 ; "TBAF" ; Nc1nc(CO)nc2c1C1(CCCC1)C(=O)N2c1cnc(N2CC3CC2CO3)nc1 ; N#CN1CCC[C@@H]1C(F)(F)F ; N=C(OCc1nc(N)c2c(n1)N(c1cnc(N3CC4CC3CO4)nc1)C(=O)C21CCCC1)N1CCC[C@@H]1C(F)(F)F]

Compound E98 was synthesized as compound E37. LCMS (ESI): t_{R}=2.589, m/z, 574.6 [M+H]⁺; ¹H NMR (400 MHz, DMSO-d₆) δ 8.39-8.36 (m, 2H), 6.59 (s, 2H), 5.80 (s, 1H), 4.99-4.95 (m, 2H), 4.89-4.85 (m, 1H), 4.70-4.69 (m, 1H), 4.53-4.49 (m, 1H), 3.83-3.81 (m, 1H), 3.68-3.66 (m, 1H), 3.51-3.48 (m, 1H), 3.41-3.38 (m, 1H), 3.33-3.30 (m, 2H), 2.15-2.12 (m, 2H), 1.98-1.81 (m, 12H).

### Example 176. Preparation of Compound E99

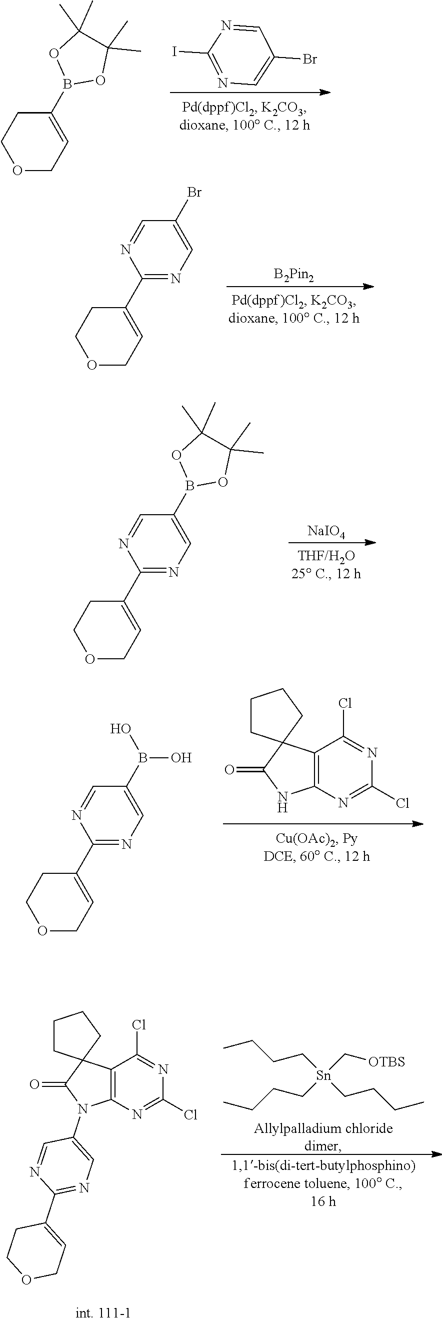

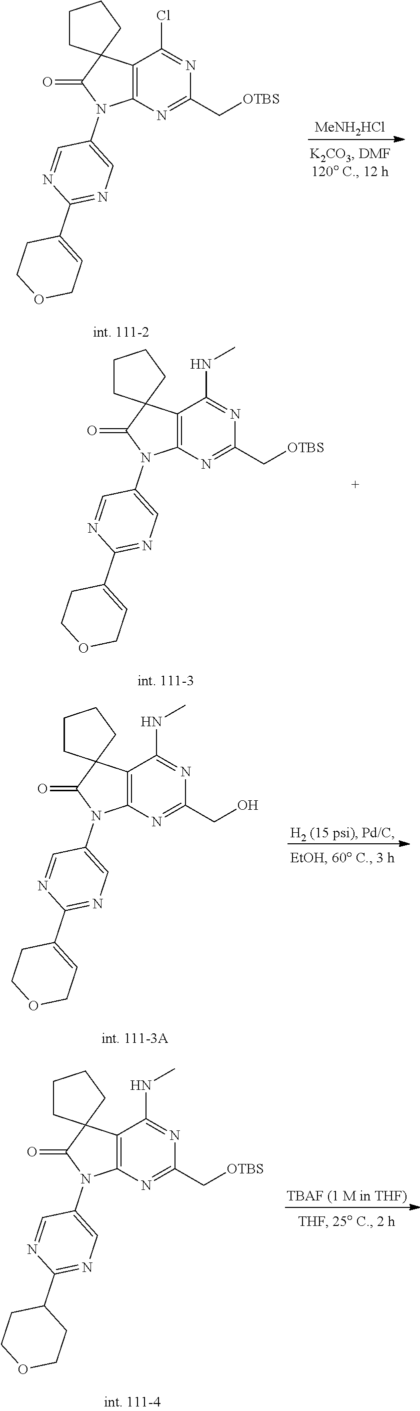 [SMILES: "MeNH2HCl" ; CC(C)(C)[Si](C)(C)OCc1nc(Cl)c2c(n1)N(c1cnc(C3=CCOCC3)nc1)C(=O)C21CCCC1 ; CNc1nc(CO[Si](C)(C)C(C)(C)C)nc2c1C1(CCCC1)C(=O)N2c1cnc(C2=CCOCC2)nc1 ; CNc1nc(CO)nc2c1C1(CCCC1)C(=O)N2c1cnc(C2=CCOCC2)nc1 ; CNc1nc(CO[Si](C)(C)C(C)(C)C)nc2c1C1(CCCC1)C(=O)N2c1cnc(C2CCOCC2)nc1]

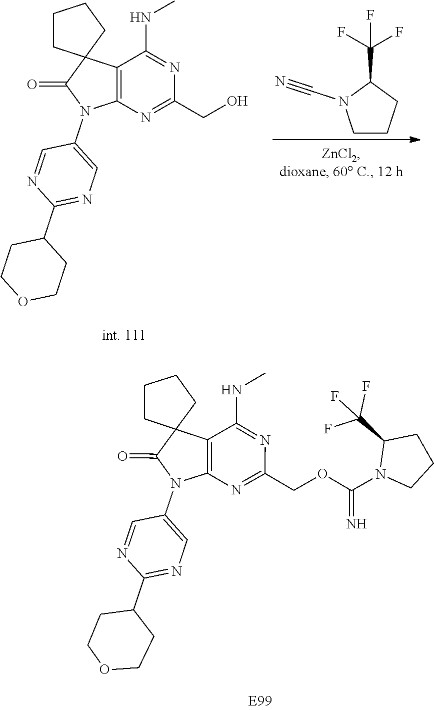 [SMILES: CNc1nc(CO)nc2c1C1(CCCC1)C(=O)N2c1cnc(C2CCOCC2)nc1 ; N#CN1CCC[C@@H]1C(F)(F)F ; CNc1nc(COC(=N)N2CCC[C@@H]2C(F)(F)F)nc2c1C1(CCCC1)C(=O)N2c1cnc(C2CCOCC2)nc1]

1. Synthesis of compound 5-bromo-2-(3,6-dihydro-2H-pyran-4-yl)pyrimidine. A mixture of 2-(3,6-dihydro-2H-pyran-4-yl)-4,4,5,5-tetramethyl-1,3,2-dioxaborolane (10.0 g, 47.6 mmol), 5-bromo-2-iodopyrimidine (13.5 g, 47.6 mmol), Pd(dppf)Cl2 (3.48 g, 4.76 mmol), and K₃PO₄ (20.2 g, 95.2 mmol) in 1,4-dioxane (100 mL) and H₂O (20 mL). The mixture was stirred at 100° C. for 12 h under N₂ atmosphere. The mixture was concentrated. The residue was purified by column chromatography to afford title compound (7.70 g). LCMS (ESI): m/z, 240.9 [M+H]⁺.

2. Synthesis of compound 2-(3,6-dihydro-2H-pyran-4-yl)-5-(4,4,5,5-tetramethyl-1,3,2-dioxaborolan-2-yl)pyrimidine. A mixture of Pin2B2 (8.03 g, 31.6 mmol), 5-bromo-2-(3,6-dihydro-2H-pyran-4-yl)pyrimidine (7.70 g, 28.7 mmol), KOAc (8.46 g, 86.2 mmol) and Pd(dppf)Cl₂ (2.10 g, 2.87 mmol) in 1,4-dioxane (100 mL) was stirred at 100° C. for 12 h under N₂ atmosphere. The mixture was filtered and the filtrate was concentrated. The residue was purified by column chromatography to afford title compound (7.0 g). LCMS (ESI): m/z, 207.1 [M+H]⁺.

3. Synthesis of compound (2-(3,6-dihydro-2H-pyran-4-yl)pyrimidin-5-yl)boronic acid. To a solution of 2-(3,6-dihydro-2H-pyran-4-yl)-5-(4,4,5,5-tetramethyl-1,3,2-dioxaborolan-2-yl)pyrimidine (8.0 g, 27.8 mmol) in H₂O (20 mL) and THF (60 mL) was added sodium periodate (17.8 g, 83.3 mmol). The mixture was stirred at 25° C. for 12 h. The mixture was filtered, and the filtrate was extracted with EA. The combined organic phases were concentrated to afford title compound (4.30 g). ¹H NMR (400 MHz, DMSO-d₆) δ 8.98 (s, 2H), 8.55 (brs, 2H), 7.26-7.25 (m, 1H), 4.31-4.29 (m, 2H), 3.81 (t, J=5.2 Hz, 2H), 2.59-2.55 (m, 2H). LCMS (ESI): m/z, 206.8 [M+H]⁺.

4. Synthesis of compound int. 111-1. A mixture of 2′,4′-dichloro-6′,7′-dihydrospiro[cyclopentane-1,5′-pyrrolo[2,3-d]pyrimidin]-6′-one (3.0 g, 11.6 mmol), [2-(3,6-dihydro-2H-pyran-4-yl)pyrimidin-5-yl]boronic acid (2.87 g, 13.9 mmol), pyridine (1.88 mL), Cu(OAc)₂ (2.11 g, 11.6 mmol), and 4A Molecular Sieve (10.2 g) in DCE (5 mL) was stirred at 60° C. for 12 h under 02 atmosphere. The mixture was filtered and the filtrate was concentrated. The residue was purified by column chromatography to afford title compound (2.34 g). LCMS (ESI): m/z, 418.1 [M+H]⁺.

5. Synthesis of compound int. 111-2. A mixture of 22′,4′-dichloro-7′-(2-(3,6-dihydro-2H-pyran-4-yl)pyrimidin-5-yl)spiro[cyclopentane-1,5′-pyrrolo[2,3-d]pyrimidin]-6′(7′H)-one (2.35 g, 5.06 mmol), (tert-butyldimethylsilyloxymethyl)tri-n-butyltin (2.20 g, 5.06 mmol), [PdCl(allyl)]₂ (185 mg, 0.505 mmol), and 1,1′-bis(di-tert-butylphosphino)ferrocene (479 mg, 1.01 mmol) in PhMe (30 mL) was stirred at 100° C. for 16 h under N₂ atmosphere. The mixture was concentrated. The residue was purified by column chromatography to afford title compound (1.80 g). LCMS (ESI): m/z, 528.2 [M+H]⁺.

6. Synthesis of compound int. 111-3 and int. 111-3A. To a solution of 2′-(((tert-butyldimethylsilyl)oxy)methyl)-4′-chloro-7′-(2-(3,6-dihydro-2H-pyran-4-yl)pyrimidin-5-yl)spiro[cyclopentane-1,5′-pyrrolo[2,3-d]pyrimidin]-6′(7′H)-one (900 mg, 1.70 mmol) and Methanamine, hydrochloride (575 mg, 8.52 mmol) in DMA (10 mL) was added K₂CO₃ (1.18 g, 8.52 mmol). The mixture was stirred at 120° C. for 12 h. The mixture was diluted with water, extracted with EA. The combined organic phases were washed with brine, dried over Na₂SO₄, filtered and concentrated. The residue was purified by column chromatography to afford 2′-(((tert-butyldimethylsilyl)oxy)methyl)-7′-(2-(3,6-dihydro-2H-pyran-4-yl)pyrimidin-5-yl)-4′-(methylamino)spiro[cyclopentane-1,5′-pyrrolo[2,3-d]pyrimidin]-6′(7′H)-one (420 mg) and 7′-(2-(3,6-dihydro-2H-pyran-4-yl)pyrimidin-5-yl)-2′-(hydroxymethyl)-4′-(methylamino)spiro[cyclopentane-1,5′-pyrrolo[2,3-d]pyrimidin]-6′(7′H)-one (200 mg). LCMS (ESI): m/z, 409.5 [M+H]⁺; LCMS (ESI): m/z, 523.6 [M+H]⁺.

7. Synthesis of compound int. 111-4. Compound int. 111-4 was synthesized as compound int. 112. LCMS (ESI): m/z, 525.3 [M+H]⁺.

8. Synthesis of compound int. 111. Compound int. 111 was synthesized as compound int. 112-2. LCMS (ESI): m/z, 411.1 [M+H]⁺.

9. Synthesis of compound E99. Compound E99 was synthesized as compound E16. ¹H NMR (400 MHz, CDCl₃) (8.99 (s, 2H), 5.27-5.11 (m, 2H), 4.52-4.50 (m, 1H), 4.37-4.36 (m, 1H), 4.12-4.08 (m, 2H), 3.61-3.54 (m, 4H), 3.30-3.21 (m, 1H), 3.11 (d, J=4.8 Hz, 3H), 2.18-1.91 (m, 16H). LCMS (ESI): m/z, 575.7 [M+H]⁺.

### Example 177. Preparation of Compound E100

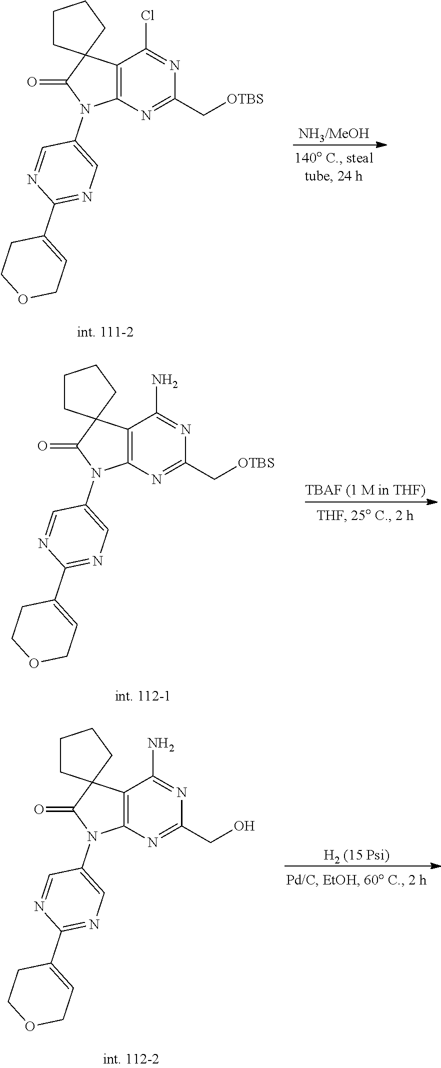 [SMILES: CC(C)(C)[Si](C)(C)OCc1nc(Cl)c2c(n1)N(c1cnc(C3=CCOCC3)nc1)C(=O)C21CCCC1 ; CC(C)(C)[Si](C)(C)OCc1nc(N)c2c(n1)N(c1cnc(C3=CCOCC3)nc1)C(=O)C21CCCC1 ; Nc1nc(CO)nc2c1C1(CCCC1)C(=O)N2c1cnc(C2=CCOCC2)nc1]

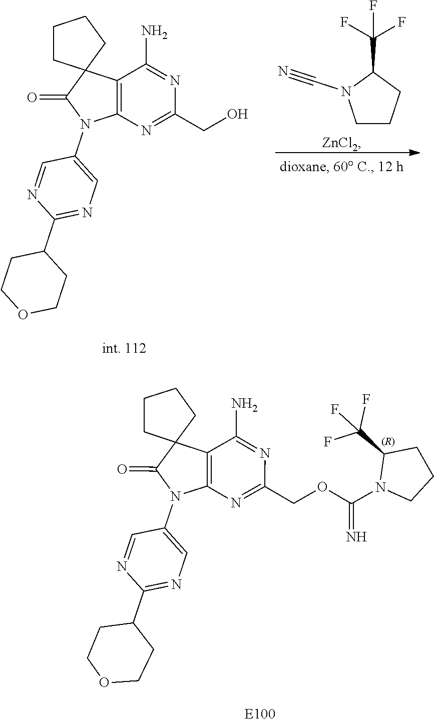

1. Synthesis of compound int. 112-1. A solution of 2′-(((tert-butyldimethylsilyl)oxy)methyl)-4′-chloro-7′-(2-(3,6-dihydro-2H-pyran-4-yl)pyrimidin-5-yl)spiro[cyclopentane-1,5′-pyrrolo[2,3-d]pyrimidin]-6′(7′H)-one (900 mg, 1.70 mmol) in NH₃ (12.2 mL, 7 M in MeOH) was stirred at 140° C. for 24 h under sealed tube. The mixture was concentrated to afford 4′-amino-2′-(((tert-butyldimethylsilyl)oxy)methyl)-7′-(2-(3,6-dihydro-2H-pyran-4-yl)pyrimidin-5-yl)spiro[cyclopentane-1,5′-pyrrolo[2,3-d]pyrimidin]-6′(7′H)-one (500 mg, purity: 50%) and 4′-amino-2′-{[(tert-butyldimethylsilyl)oxy]methyl}-7′-[2-(3,6-dihydro-2H-pyran-4-yl)pyrimidin-5-yl]-6′,7′-dihydrospiro[cyclopentane-1,5′-pyrrolo[2,3-d]pyrimidin]-6′-one (500 mg, purity: 50%). LCMS (EST): m/z, 509.2 [M+H]⁺.

2. Synthesis of compound int. 112-2. To a solution of 4′-amino-7′-[2-(3,6-dihydro-2H-pyran-4-yl)pyrimidin-5-yl]-2′-(hydroxymethyl)-6′,7′-dihydrospiro[cyclopentane-1,5′-pyrrolo[2,3-d]pyrimidin]-6′-one (500 mg, 50% purity, 0.633 mmol) and 4′-amino-2′-{[(tert-butyldimethylsilyl)oxy]methyl}-7′-[2-(3,6-dihydro-2H-pyran-4-yl)pyrimidin-5-yl]-6′,7′-dihydrospiro[cyclopentane-1,5′-pyrrolo[2,3-d]pyrimidin]-6′-one (500 mg, 50%, 491 mmol) in THF (10 mL) was added TBAF (1.90 mL, 1 M in THF). The mixture was stirred at 25° C. for 2 h. The mixture was concentrated. The residue was purified by column chromatography to afford 4′-amino-7′-(2-(3,6-dihydro-2H-pyran-4-yl)pyrimidin-5-yl)-2′-(hydroxymethyl)spiro[cyclopentane-1,5′-pyrrolo[2,3-d]pyrimidin]-6′(7′H)-one (500 mg). LCMS (ESI): m/z, 395.2 [M+H]⁺.

3. Synthesis of compound int. 112. To a solution of 4′-amino-7′-(2-(3,6-dihydro-2H-pyran-4-yl)pyrimidin-5-yl)-2′-(hydroxymethyl)spiro[cyclopentane-1,5′-pyrrolo[2,3-d]pyrimidin]-6′(7′H)-one (555 mg, 1.27 mmol) in EtOH (10 mL) was added Pd/C (404 mg, 3.80 mmol).The mixture was stirred at 60° C. for 2 h under H₂ atmosphere (15 psi). The mixture was filtered. The filtrate was concentrated to afford 4′-amino-2′-(hydroxymethyl)-7′-(2-(tetrahydro-2H-pyran-4-yl)pyrimidin-5-yl)spiro[cyclopentane-1,5′-pyrrolo[2,3-d]pyrimidin]-6′(7′H)-one (500 mg). LCMS (ESI): m/z, 397.1 [M+H]⁺.

4. Synthesis of compound E100. To a solution of (2R)-2-(trifluoromethyl)pyrrolidine-1-carbonitrile (298 mg, 1.82 mmol), 4′-amino-2′-(hydroxymethyl)-7′-[2-(oxan-4-yl)pyrimidin-5-yl]-6′,7′-dihydrospiro[cyclopentane-1,5′-pyrrolo[2,3-d]pyrimidin]-6′-one (400 mg, 0.908 mmol) in 1,4-dioxane (10 mL) was stirred at 60° C. for 12 h under N₂ atmosphere. The mixture was concentrated. The residue was purified by Pre-HPLC to afford (4′-amino-6′-oxo-7′-(2-(tetrahydro-2H-pyran-4-yl)pyrimidin-5-yl)-6′,7′-dihydrospiro[cyclopentane-1,5′-pyrrolo[2,3-d]pyrimidin]-2′-yl)methyl (R)-2-(trifluoromethyl)pyrrolidine-1-carbimidate (220 mg). ¹H NMR (400 MHz, CDCl₃) δ 8.99 (s, 2H), 5.20-5.05 (m, 2H), 4.86 (s, 2H), 4.46-4.42 (m, 1H), 4.12-4.08 (m, 2H), 3.61-3.46 (m, 4H), 3.19-3.12 (m, 1H), 2.20-1.93 (m, 16H). LCMS (ESI): m/z, 561.6 [M+H]⁺.

### Example 178. Preparation of Compound E101

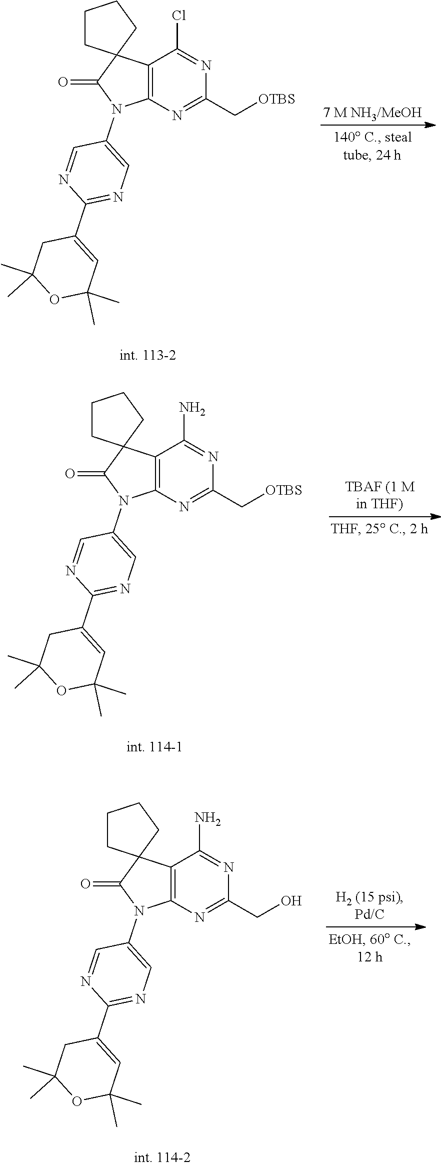 [SMILES: CC1(C)C=C(c2ncc(N3C(=O)C4(CCCC4)c4c(Cl)nc(CO[Si](C)(C)C(C)(C)C)nc43)cn2)CC(C)(C)O1 ; CC1(C)C=C(c2ncc(N3C(=O)C4(CCCC4)c4c(N)nc(CO[Si](C)(C)C(C)(C)C)nc43)cn2)CC(C)(C)O1 ; CC1(C)C=C(c2ncc(N3C(=O)C4(CCCC4)c4c(N)nc(CO)nc43)cn2)CC(C)(C)O1]

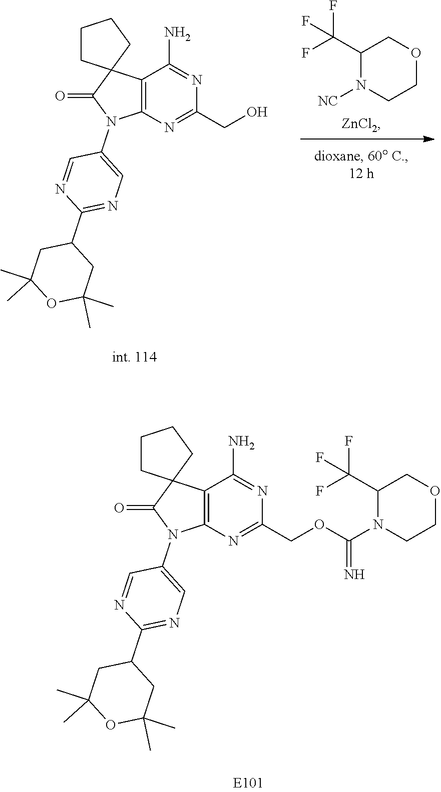 [SMILES: CC1(C)CC(c2ncc(N3C(=O)C4(CCCC4)c4c(N)nc(CO)nc43)cn2)CC(C)(C)O1 ; N#CN1CCOCC1C(F)(F)F ; CC1(C)CC(c2ncc(N3C(=O)C4(CCCC4)c4c(N)nc(COC(=N)N5CCOCC5C(F)(F)F)nc43)cn2)CC(C)(C)O1]

Compound E101 was synthesized as compound E100. ¹H NMR (400 MHz, CDCl₃) δ 8.99 (s, 2H), 5.14-4.98 (m, 2H), 4.92 (s, 2H), 4.55-4.52 (m, 1H), 4.19-4.16 (m, 1H), 3.97-3.94 (m, 1H), 3.75-3.36 (m, 5H), 2.22-2.10 (m, 8H), 1.96-1.91 (m, 2H), 1.74-1.68 (m, 2H), 1.38 (s, 6H), 1.28 (s, 6H). LCMS (ESI): m/z, 633.7 [M+H]⁺.

### Example 179. Preparation of Compound E102

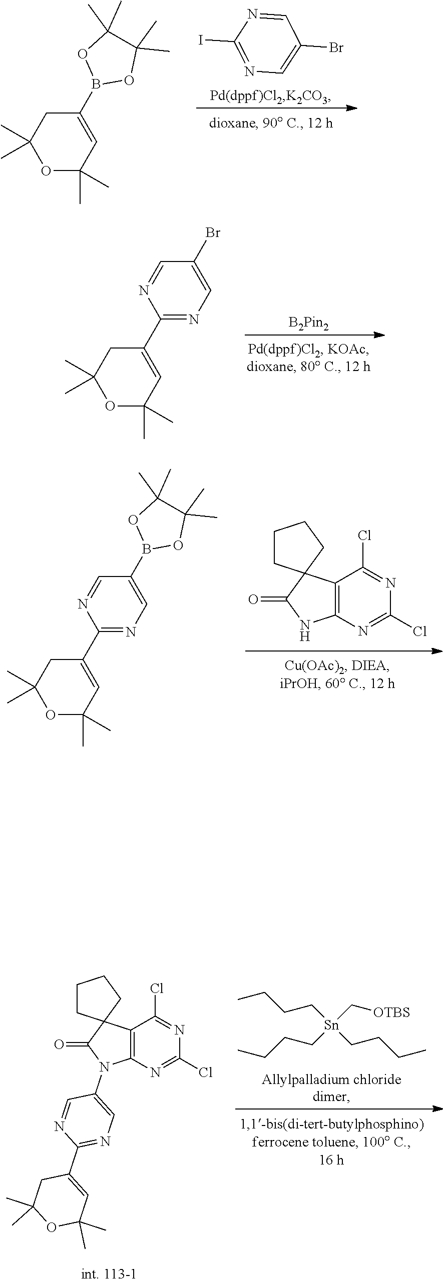

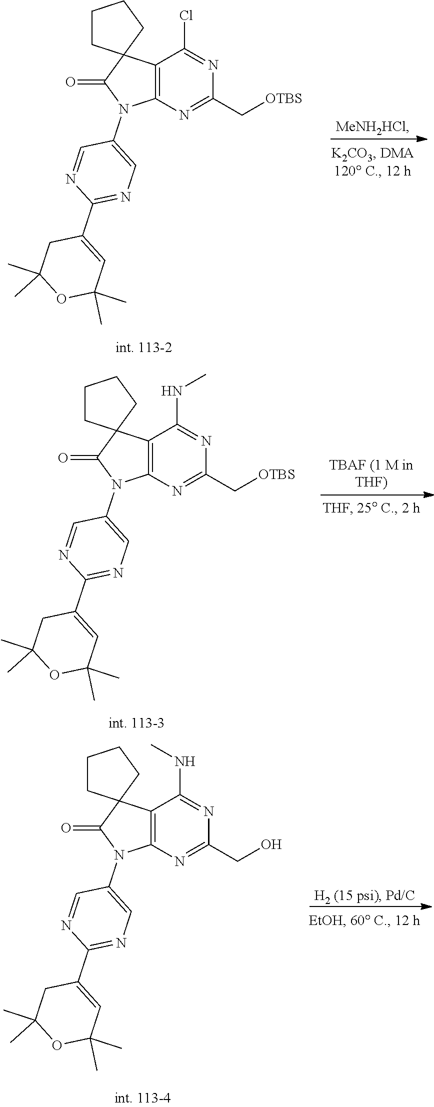 [SMILES: CC1(C)C=C(c2ncc(N3C(=O)C4(CCCC4)c4c(Cl)nc(CO[Si](C)(C)C(C)(C)C)nc43)cn2)CC(C)(C)O1 ; "MeNH2HCl," ; CNc1nc(CO[Si](C)(C)C(C)(C)C)nc2c1C1(CCCC1)C(=O)N2c1cnc(C2=CC(C)(C)OC(C)(C)C2)nc1 ; CNc1nc(CO)nc2c1C1(CCCC1)C(=O)N2c1cnc(C2=CC(C)(C)OC(C)(C)C2)nc1]

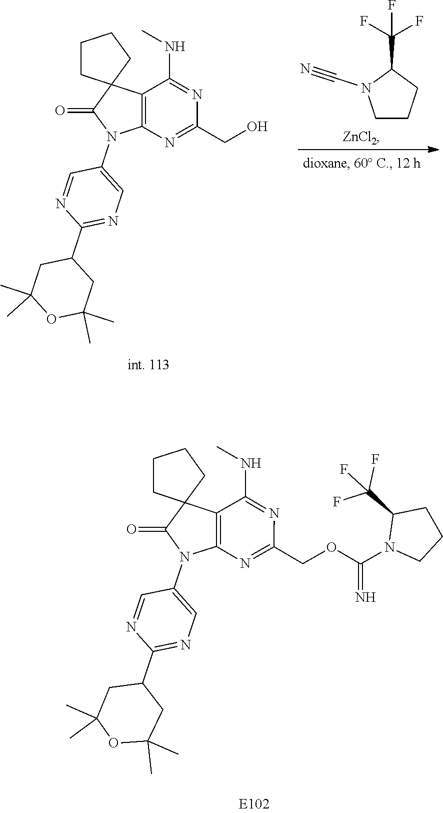 [SMILES: CNc1nc(CO)nc2c1C1(CCCC1)C(=O)N2c1cnc(C2CC(C)(C)OC(C)(C)C2)nc1 ; N#CN1CCC[C@@H]1C(F)(F)F ; CNc1nc(COC(=N)N2CCC[C@@H]2C(F)(F)F)nc2c1C1(CCCC1)C(=O)N2c1cnc(C2CC(C)(C)OC(C)(C)C2)nc1]

1. Synthesis of compound 5-bromo-2-(2,2,6,6-tetramethyl-3,6-dihydro-2H-pyran-4-yl)pyrimidine. A mixture of 4,4,5,5-tetramethyl-2-(2,2,6,6-tetramethyl-3,6-dihydro-2H-pyran-4-yl)-1,3,2-dioxaborolane (960 mg, 3.61 mmol), 5-bromo-2-iodopyrimidine (1.34 g, 4.69 mmol), Pd(dppf)Cl₂ (263 mg, 0.360 mmol), K₃PO₄ (2.30 g, 10.8 mmol) in 1,4-dioxane (10 mL) was stirred at 90° C. for 12 h under N₂ atmosphere. The mixture was concentrated. The residue was purified by column chromatography to afford title compound (800 mg). LCMS (ESI): m/z, 297.2 [M+H]⁺.

2. Synthesis of compound 5-(4,4,5,5-tetramethyl-1,3,2-dioxaborolan-2-yl)-2-(2,2,6,6-tetramethyl-3,6-dihydro-2H-pyran-4-yl)pyrimidine. A mixture of 5-bromo-2-(2,2,6,6-tetramethyl-3,6-dihydro-2H-pyran-4-yl)pyrimidine (800 mg, 2.42 mmol), Pin₂B₂ (861 mg, 3.39 mmol), KOAc (475 mg, 4.85 mmol), and Pd(dppf)Cl₂ (354 mg, 0.484 mmol) in 1,4-dioxane (15 mL) was stirred at 90° C. for 12 h under N₂ atmosphere. The mixture was concentrated. The residue was purified by column chromatography to afford title compound (380 mg). ¹H NMR (400 MHz, CDCl₃) δ 8.96 (s, 2H), 7.28-7.26 (m, 1H), 2.58-2.57 (m, 2H), 1.35 (s, 12H), 1.31 (s, 6H), 1.26 (s, 6H).

3. Synthesis of compound int. 113-1. A mixture of 2′,4′-dichloro-6′,7′-dihydrospiro[cyclopentane-1,5′-pyrrolo[2,3-d]pyrimidin]-6′-one (3.0 g, 11.6 mmol), 5-(4,4,5,5-tetramethyl-1,3,2-dioxaborolan-2-yl)-2-(2,2,6,6-tetramethyl-3,6-dihydro-2H-pyran-4-yl)pyrimidine (4.40 g, 12.8 mmol), Cu(OAc)₂·H₂O (2.32 g, 11.62 mmol), 4A Molecular Sieve (10.2 g), and DIPEA (3.84 mL) in i-PrOH (60 mL) was stirred at 60° C. for 12 h under 02 atmosphere. The mixture was filtered, and the filtrate was concentrated. The residue was purified by column chromatography to afford title compound (2.10 g). LCMS (ESI): m/z, 374.1 [M+H]⁺.

4. Synthesis of compound int. 113-2. A mixture of 2′,4′-dichloro-7′-[2-(2,2,6,6-tetramethyl-3,6-dihydro-2H-pyran-4-yl)pyrimidin-5-yl]-6′,7′-dihydrospiro[cyclopentane-1,5′-pyrrolo[2,3-d]pyrimidin]-6′-one (2.25 g, 4.74 mmol), (tert-butyldimethylsilyloxymethyl)tri-n-butyltin (2.06 g, 4.74 mmol), 1,1′-bis(di-tert-butylphosphino)ferrocene (450 mg, 0.948 mmol), [PdCl(allyl)]₂ (173 mg, 0.474 mmol) in PhMe (50 mL) was stirred at 100° C. for 12 h under N₂ atmosphere. The mixture was concentrated. The residue was purified by column chromatography to afford title compound (1.80 g). LCMS (ESI): m/z, 584.3 [M+H]⁺.

5. Synthesis of compound int. 113-3. To a solution of 2′-(((tert-butyldimethylsilyl)oxy)methyl)-4′-chloro-7′-(2-(2,2,6,6-tetramethyl-3,6-dihydro-2H-pyran-4-yl)pyrimidin-5-yl)spiro[cyclopentane-1,5′-pyrrolo[2,3-d]pyrimidin]-6′(7′H)-one (850 mg, 1.45 mmol) in DMA (10 mL) was added methanamine, hydrochloride (491 mg, 7.27 mmol) and K₂CO₃ (1.01 g, 7.27 mmol). The mixture was stirred at 120° C. for 12 h. The mixture was diluted with water, extracted with EA, washed with sat. aq. NaCl, dried over Na₂SO₄, filtered and concentrated to afford title compound (800 mg crude). LCMS (ESI): m/z, 579.3 [M+H]⁺.

6. Synthesis of compound int. 113-4. To a solution of 2′-(((tert-butyldimethylsilyl)oxy)methyl)-4′-(methylamino)-7′-(2-(2,2,6,6-tetramethyl-3,6-dihydro-2H-pyran-4-yl)pyrimidin-5-yl)spiro[cyclopentane-1,5′-pyrrolo[2,3-d]pyrimidin]-6′(7′H)-one (800 mg, 1.38 mmol) in THF (10 mL) was added TBAF (4.15 mL, 1 M in THF). The mixture was stirred at 25° C. for 2 h. The mixture was concentrated. The residue was purified by column chromatography to afford title compound (300 mg). LCMS (ESI): m/z, 465.3 [M+H]⁺.

7. Synthesis of compound int. 113. To a solution of 2′-(hydroxymethyl)-4′-(methylamino)-7′-(2-(2,2,6,6-tetramethyl-3,6-dihydro-2H-pyran-4-yl)pyrimidin-5-yl)spiro[cyclopentane-1,5′-pyrrolo[2,3-d]pyrimidin]-6′(7′H)-one (300 mg, 0.645 mmol) in EtOH (10 mL) was added Pd/C (6 mg, 0.645 mmol). The mixture was stirred at 60° C. for 12 h under H₂ atmosphere. The mixture was filtered and concentrated to afford title compound (250 mg). LCMS (ESI): m/z, 467.2 [M+H]⁺.

8. Synthesis of compound E102. To a solution of (2R)-2-(trifluoromethyl)pyrrolidine-1-carbonitrile (246 mg, 1.50 mmol), 2′-(hydroxymethyl)-4′-(methylamino)-7′-[2-(2,2,6,6-tetramethyloxan-4-yl)pyrimidin-5-yl]-6′,7′-dihydrospiro[cyclopentane-1,5′-pyrrolo[2,3-d]pyrimidin]-6′-one (280 mg, 0.600 mmol) in 1,4-dioxane (10 mL) was added ZnCl₂ (163 mg, 51.2 mmol). The mixture was stirred at 60° C. for 12 h under N₂ atmosphere. The mixture was concentrated. The residue was purified by Pre-HPLC to afford title compound (150 mg). ¹H NMR (400 MHz, CDCl₃) δ 9.01 (s, 2H), 5.26-5.10 (m, 2H), 4.56-4.47 (m, 1H), 4.36-4.35 (m, 1H), 3.59-3.44 (m, 3H), 3.10 (d, J=9.2 Hz, 3H), 2.20-2.02 (m, 10H), 1.94-1.90 (m, 4H), 1.74-1.67 (m, 2H), 1.38 (s, 6H), 1.28 (s, 6H). LCMS (ESI): m/z, 631.3 [M+H]⁺.

### Example 180. Preparation of Compound E103

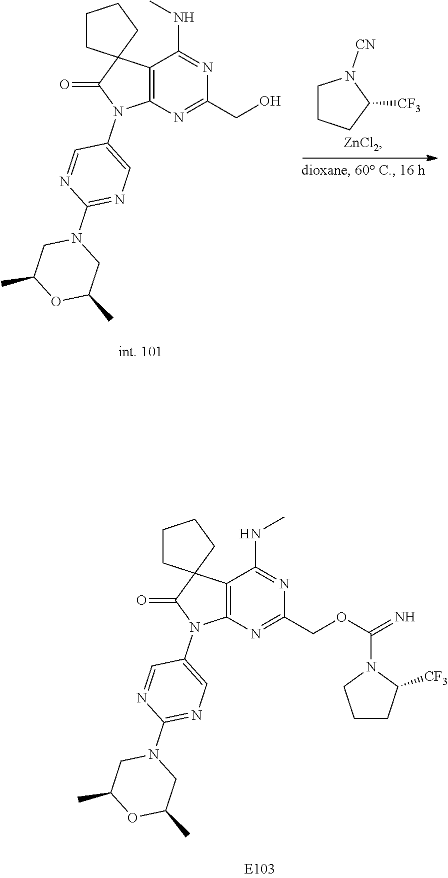 [SMILES: CNc1nc(CO)nc2c1C1(CCCC1)C(=O)N2c1cnc(N2C[C@@H](C)O[C@@H](C)C2)nc1 ; N#CN1CCC[C@H]1C(F)(F)F ; CNc1nc(COC(=N)N2CCC[C@H]2C(F)(F)F)nc2c1C1(CCCC1)C(=O)N2c1cnc(N2C[C@@H](C)O[C@@H](C)C2)nc1]

Compound E103 was synthesized as compound E16. ¹H NMR (400 MHz, CDCl₃) δ 8.45 (s, 2H), 5.26-4.97 (m, 2H), 4.68-4.53 (m, 2H), 4.52-4.44 (m, 1H), 4.28 (q, J=4.8 Hz, 1H), 3.67-3.59 (m, 2H), 3.53-3.45 (m, 2H), 3.08 (d, J=4.8 Hz, 3H), 2.62 (dd, J=10.4, 13.2 Hz, 2H), 2.21-2.03 (m, 7H), 2.01-1.85 (m, 5H), 1.26 (d, J=6.0 Hz, 6H). LCMS (ESI): m/z, 604.3 [M+H]⁺.

### Example 181. Preparation of Compound E104, E104A and E104B

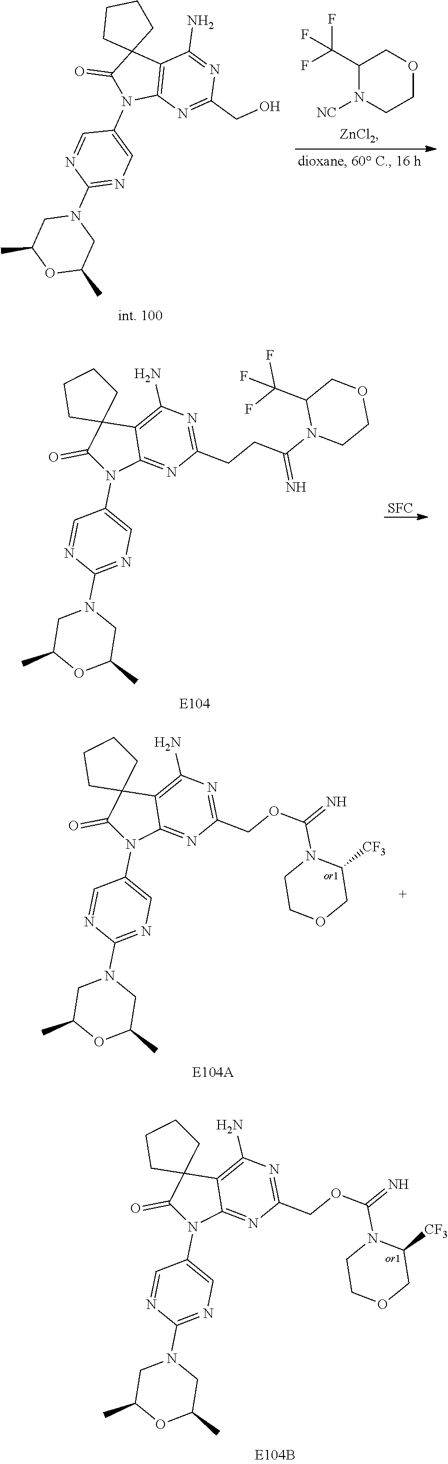 [SMILES: C[C@@H]1CN(c2ncc(N3C(=O)C4(CCCC4)c4c(N)nc(CO)nc43)cn2)C[C@H](C)O1 ; N#CN1CCOCC1C(F)(F)F ; C[C@@H]1CN(c2ncc(N3C(=O)C4(CCCC4)c4c(N)nc(CCC(=N)N5CCOCC5C(F)(F)F)nc43)cn2)C[C@H](C)O1 ; "SFC" ; C[C@@H]1CN(c2ncc(N3C(=O)C4(CCCC4)c4c(N)nc(COC(=N)N5CCOC[C@H]5C(F)(F)F)nc43)cn2)C[C@H](C)O1 ; C[C@@H]1CN(c2ncc(N3C(=O)C4(CCCC4)c4c(N)nc(COC(=N)N5CCOC[C@@H]5C(F)(F)F)nc43)cn2)C[C@H](C)O1]

Compound E104 was synthesized as compound E16. ¹H NMR (400 MHz, CDCl₃) δ 8.44 (s, 2H), 5.13-4.91 (m, 2H), 4.79 (s, 2H), 4.70-4.44 (m, 3H), 4.15 (d, J=12.4 Hz, 1H), 3.97-3.89 (m, 1H), 3.76-3.50 (m, 5H), 3.38 (t, J 12.4 Hz, 1H), 2.64 (dd, J 10.4, 13.2 Hz, 2H), 2.16 (dd, J 6.8, 11.2 Hz, 4H), 2.09 (d, J=7.6 Hz, 2H), 1.92 (s, 2H), 1.26 (d, J=6.0 Hz, 6H). LCMS (ESI): m/z, 606.3 [M+H]⁺. The compound E104 was set to SFC separation (Column name: DAICELCHIRALPAK®IG; Mobile Phase A: Supercritical CO₂; Mobile Phase B: IPA; Wavelength: 214 nm; Flow: 1.5 mL/min; Column temp: 35° C.) to get 2 compounds; corresponding to the ret. time (min): 3.940 (P1), 4.910 (P2); ratio is 1:1. LCMS(P1) (ESI): t_{R}=3.116, m/z, 606.6 [M+H]⁺; (P2) LCMS(P1) (ESI): t_{R}=3.115, m/z, 606.6 [M+H]⁺.

### Example 182. Preparation of Compound E105 and Other Compounds

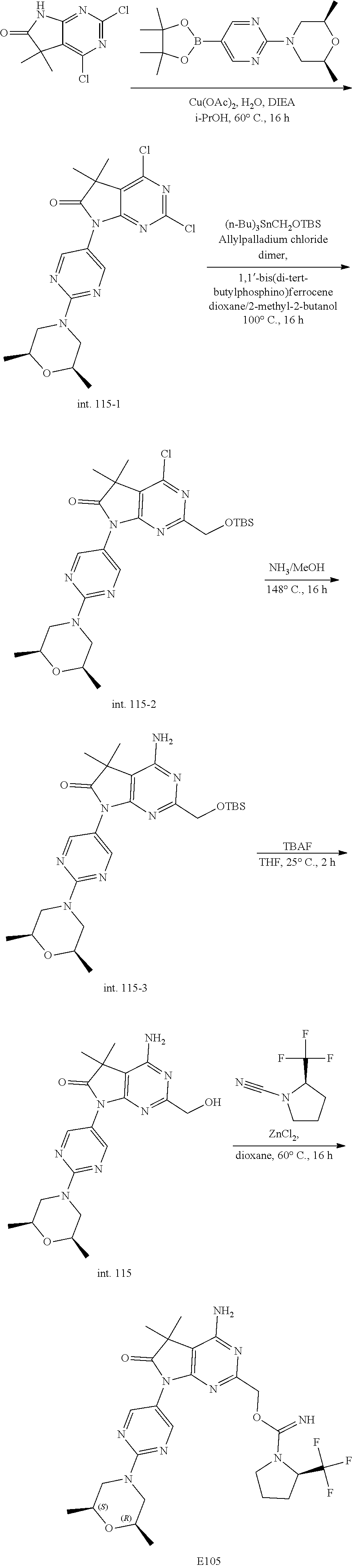

Compound E105 was synthesized as compound E93. LCMS (ESI): m/z, 564.8 [M+H]; ¹H NMR (400 MHz, DMSO-d₆) δ 8.42 (s, 2H), 6.81 (s, 2H), 5.78 (s, 1H), 5.02-4.87 (m, 2H), 4.54-4.50 (m, 3H), 3.59-3.53 (m, 2H), 3.31-3.19 (m, 2H), 2.60-2.54 (m, 2H), 1.89-1.83 (m, 4H), 1.42 (s, 6H), 1.16 (d, J=6.4 Hz, 6H).

Additional compounds were synthesized as compound E105, for example:

| Compound | Structure | LCMS, NMR |
|---|---|---|
| E106 | 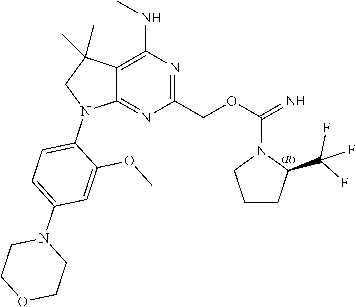 | ¹H NMR (400 MHz, CDCl₃) δ 7.24 (d, J = 8.4 Hz, 1H), 6.49-6.46 (m, 2H), 5.08-4.88 (m, 2H), 4.58-4.54 (m, 1H), 4.14-4.12 (m, 1H), 3.86 (t, J = 4.8 Hz, 4H), 3.77 (s, 3H), 3.57-3.45 (m, 4H), 3.15 (t, J = 4.8 Hz, 4H), 3.02 (d, J = 4.8 Hz, 3H), 2.15-2.00 (m, 2H), 1.89- 1.85 (m, 2H), 1.37 (s, 6H). LCMS (ESI): m/z, 564.4 [M + H]⁺. |
| E107 | 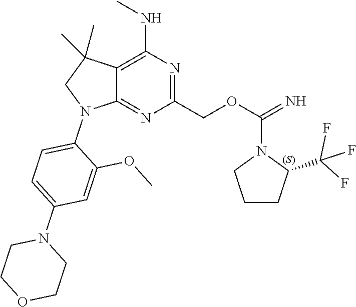 | ¹H NMR (400 MHz, CDCl₃) δ 7.24 (d, J = 8.4 Hz, 1H), 6.49-6.46 (m, 2H), 5.07-4.87 (m, 2H), 4.58- 4.54 (m, 1H), 4.14-4.11 (m, 1H), 3.86 (t, J = 4.8 Hz, 4H), 3.77 (s, 3H), 3.57-3.44 (m, 4H), 3.15 (t, J = 4.8 Hz, 4H), 3.02 (d, J = 4.8 Hz, 3H), 2.15-2.00 (m, 2H), 1.89-1.85 (m, 2H), 1.37 (s, 6H). LCMS (ESI): m/z, 564.4 [M + H]⁺. |
| E108 | 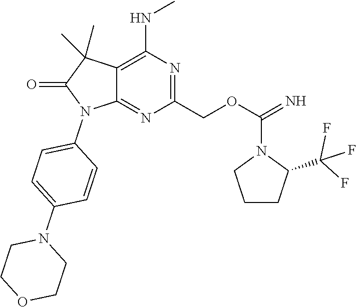 [SMILES: CNc1nc(COC(=N)N2CCC[C@H]2C(F)(F)F)nc2c1C(C)(C)C(=O)N2c1ccc(N2CCOCC2)cc1] | LCMS (ESI): m/z, 548.3 [M + H]⁺. ¹H NMR (400 MHz, CDCl₃) δ 7.39-7.32 (m, 2H), 6.98-6.92 (m, 2H), 5.21 (d, J = 14.8 Hz, 1H), 5.02 (d, J = 14.8 Hz, 1H), 4.49 (m, 1H), 4.37 (m, 1H), 3.89-3.84 (m, 4H), 3.44 (m, 2H), 3.21-3.15 (m, 4H), 3.08 (d, J = 4.8 Hz, 3H), 2.08- 2.01 (m, 2H), 1.94-1.86 (m, 2H), 1.48 (s, 6H). |
| E109 | 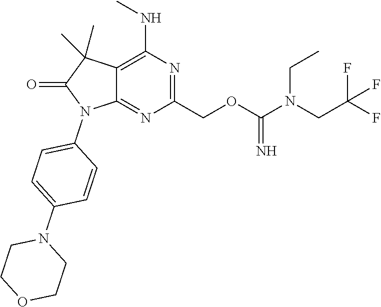 [SMILES: CCN(CC(F)(F)F)C(=N)OCc1nc(NC)c2c(n1)N(c1ccc(N3CCOCC3)cc1)C(=O)C2(C)C] | ¹H NMR (400 MHz, CDCl₃) δ 7.38-7.30 (m, 2H), 7.02- 6.92 (m, 2H), 5.07 (s, 2H), 4.38 (q, J = 4.8 Hz, 1H), 3.87 (dt, J = 2.8, 7.2 Hz, 6H), 3.38 (q, J = 7.2 Hz, 2H), 3.24-3.14 (m, 4H), 3.08 (d, J = 4.8 Hz, 3H), 1.49 (s, 6H), 1.13 (t, J = 7.2 Hz, 3H). LCMS (ESI): m/z, 536.4 [M + H]⁺. |
| E110 | 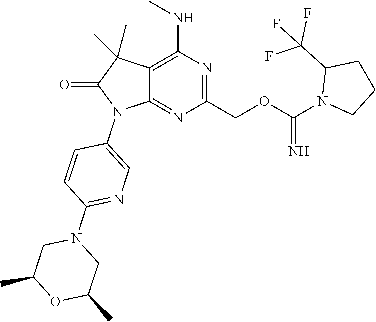 | ¹H NMR (400 MHz, CDCl₃) δ 8.29 (m, 1H), 7.62-7.60 (m, 1H), 6.69-6.67 (m, 1H), 5.22-5.20 (m, 1H), 5.06- 5.04 (m, 1H), 4.48 (t, J = 8.0 Hz, 1H), 4.41 (d, J = 5.2 Hz, 1H), 4.10-4.08 (m, 2H), 3.73-3.72 (m, 2H), 3.51- 3.40 (m, 2H), 3.08 (d, J = 4.8 Hz, 3H), 2.57-2.55 (m, 2H), 2.09-2.03 (m, 2H), 1.95-1.93 (m, 2H), 1.48 (s, 6H), 1.27 (d, J = 6.4 Hz, 6H). LCMS (ESI): m/z, 548.3 [M + H]⁺. |
| E111 | 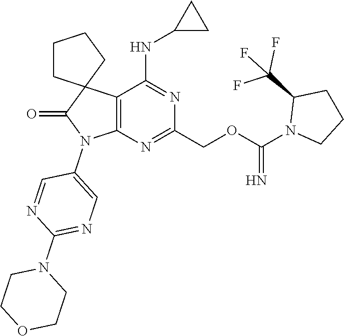 [SMILES: N=C(OCc1nc(NC2CC2)c2c(n1)N(c1cnc(N3CCOCC3)nc1)C(=O)C21CCCC1)N1CCC[C@@H]1C(F)(F)F] | ¹H NMR (400 MHz, DMSO-d₆) δ 8.42 (s, 2H), 6.38 (s, 1H), 5.75 (s, 1H), 5.06-4.93 (m, 2H), 4.56-4.52 (m, 1H), 3.75-3.66 (m, 8H), 2.86-2.82 (m, 1H), 2.16- 2.13 (m, 2H), 1.96-1.82 (m, 10H), 0.73-0.54 (m, 4H). LCMS (ESI): m/z, 602.4 [M + H]⁺. |
| E112 | 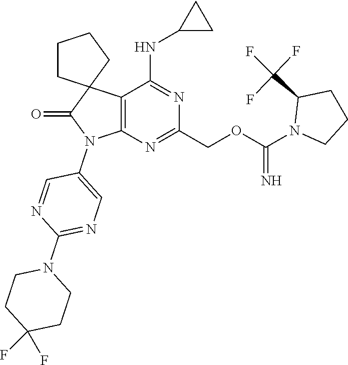 [SMILES: N=C(OCc1nc(NC2CC2)c2c(n1)N(c1cnc(N3CCC(F)(F)CC3)nc1)C(=O)C21CCCC1)N1CCC[C@@H]1C(F)(F)F] | ¹H NMR (400 MHz, DMSO-d₆) δ 8.44 (s, 2H), 6.38 (s, 1H), 5.75 (s, 1H), 5.06-4.92 (m, 2H), 4.56-4.52 (m, 1H), 3.95-3.92 (m, 4H), 2.86-2.82 (m, 1H), 2.16- 1.82 (m, 18H), 0.71-0.54 (m, 4H). LCMS (ESI): m/z, 636.3 [M + H]⁺. |
| E113 | 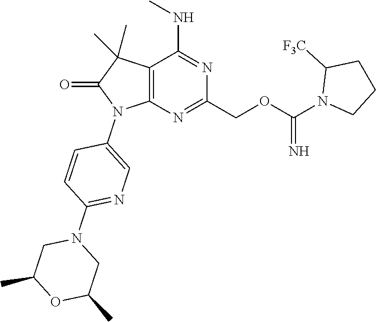 [SMILES: CNc1nc(COC(=N)N2CCCC2C(F)(F)F)nc2c1C(C)(C)C(=O)N2c1ccc(N2C[C@@H](C)O[C@@H](C)C2)nc1] | ¹H NMR (400 MHz, DMSO-d₆) δ 8.10 (d, J = 2.4 Hz, 1H), 7.55 (dd, J= 2.8, 9.2 Hz, 1H), 6.92 (d, J = 9.2 Hz, 1H), 6.72 (d, J = 5.6 Hz, 1H), 5.77 (s, 1H), 4.96 (q, J = 14.8 Hz, 2H), 4.60-4.49 (m, 1H), 4.23- 4.11 (m, 2H), 3.65-3.57 (m, 2H), 3.31 (s, 2H), 2.89 (d, J = 4.4 Hz, 3H), 2.43 (dd, J = 10.4, 12.8 Hz, 2H), 1.95-1.74 (m, 4H), 1.42 (s, 6H), 1.17 (d, J = 6.0 Hz, 6H). LCMS (ESI): m/z, 577.3 [M + H]⁺. |

Additional compounds were synthesized as compound E16, for example:

| Compound | Structure | LCMS, NMR |
|---|---|---|
| E114 | 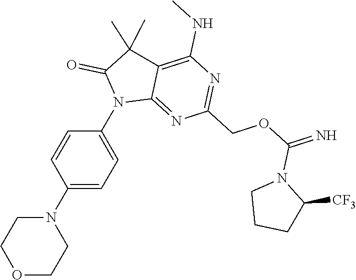 [SMILES: CNc1nc(COC(=N)N2CCC[C@@H]2C(F)(F)F)nc2c1C(C)(C)C(=O)N2c1ccc(N2CCOCC2)cc1] | ¹H NMR (400 MHz, CDCl₃) δ 7.36 (d, J = 9.2 Hz, 2H), 6.95 (d, J = 9.2 Hz, 2H), 5.26-4.96 (m, 2H), 4.49 (t, J = 8.0 Hz, 1H), 4.41-4.32 (m, 1H), 3.94-3.79 (m, 4H), 3.50-3.40 (m, 2H), 3.22-3.14 (m, 4H), 3.08 (d, J = 4.8 Hz, 3H), 2.09-2.03 (m, 2H), 1.94-1.83 (m, 2H), 1.48 (s, 6H). LCMS (ESI): m/z, 548.3 [M + H]⁺. |
| E115 | 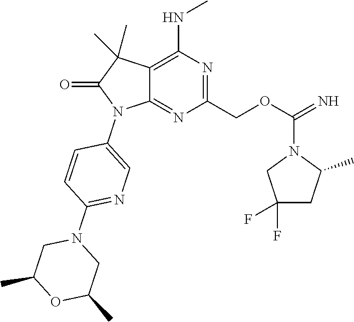 | ¹H NMR (400 MHz, CDCl₃) δ 8.31 (d, J = 2.4 Hz, 1H), 7.60 (dd, J = 2.8, 9.2 Hz, 1H), 6.67 (d, J = 9.2 Hz, 1H), 5.16-5.04 (m, 2H), 4.38 (q, J = 4.8 Hz, 1H), 4.22- 4.12 (m, 1H), 4.11-4.04 (m, 2H), 3.86-3.66 (m, 4H), 3.08 (d, J = 4.8 Hz, 3H), 2.61-2.47 (m, 3H), 2.08- 2.02 (m, 1H), 1.49 (s, 6H), 1.27 (d, J = 6.0 Hz, 9H). LCMS (ESI): m/z, 559.4 [M + H]⁺. |
| E116 | 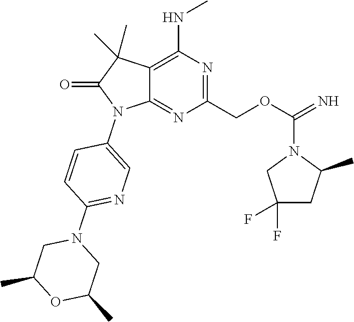 | ¹H NMR (400 MHz, CDCl₃) δ 8.31 (d, J = 2.4 Hz, 1H), 7.60 (dd, J = 2.8, 9.2 Hz, 1H), 6.67 (d, J = 9.2 Hz, 1H), 5.17-5.02 (m, 2H), 4.39 (q, J = 4.8 Hz, 1H), 4.18- 4.14 (m, 1H), 4.12-4.03 (m, 2H), 3.87-3.65 (m, 4H), 3.08 (d, J = 4.8 Hz, 3H), 2.61-2.49 (m, 3H), 2.09- 1.99 (m, 1H), 1.48 (s, 6H), 1.27 (d, J = 6.4 Hz, 9H). LCMS (ESI): m/z, 559.4 [M + H]⁺. |
| E117 | 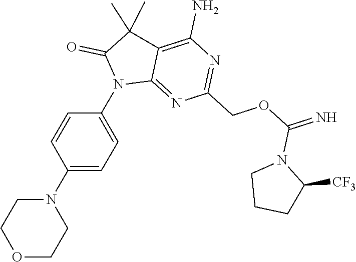 [SMILES: CC1(C)C(=O)N(c2ccc(N3CCOCC3)cc2)c2nc(COC(=N)N3CCC[C@@H]3C(F)(F)F)nc(N)c21] | ¹H NMR (400 MHz, CDCl₃) δ 7.36 (d, J = 8.8 Hz, 2H), 6.96 (d, J = 8.8 Hz, 2H), 5.21-4.99 (m, 2H), 4.82 (s, 2H), 4.53-4.41 (m, 1H), 3.92-3.82 (m, 4H), 3.48- 3.42 (m, 2H), 3.24-3.14 (m, 4H), 2.10-2.03 (m, 2H), 1.51 (s, 6H). LCMS (ESI): m/z, 534.4 [M + H]⁺. |
| E118 | 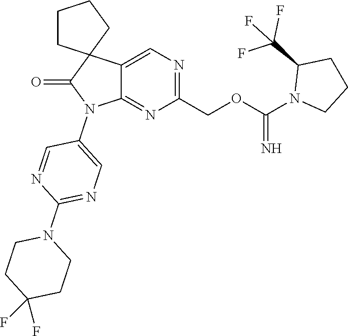 [SMILES: N=C(OCc1ncc2c(n1)N(c1cnc(N3CCC(F)(F)CC3)nc1)C(=O)C21CCCC1)N1CCC[C@@H]1C(F)(F)F] | ¹H NMR (400 MHz, DMSO-d₆) δ 8.55 (s, 1H), 8.53 (s, 2H), 5.84 (s, 1H), 5.16 (d, J = 11.2 Hz, 2H), 4.55 (s, 1H), 3.94 (d, J = 5.6 Hz, 4H), 2.12 (d, J = 8.4 Hz, 2H), 2.18-1.85 (m, 4H), 2.00-1.96 (m, 8H), 1.93 (s, 2H). LCMS (ESI): m/z, 581.3 [M + H]⁺. |

### Example 183. Preparation of Compound E119

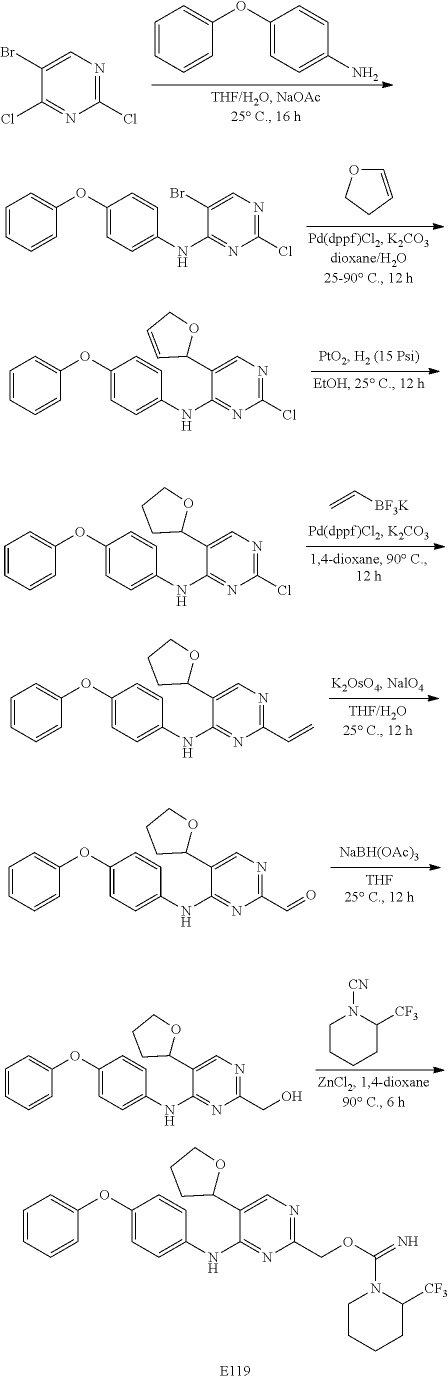

1. Synthesis of 5-bromo-2-chloro-N-(4-phenoxyphenyl)pyrimidin-4-amine. To a solution of 5-bromo-2,4-dichloro-pyrimidine (10.0 g, 43.9 mmol) was added 4-phenoxyaniline (8.1 g, 43.9 mmol) and sodium acetate trihydrate (11.9 g, 87.8 mmol) in the mixture of H₂O (50 mL) and tetrahydrofuran (50 mL). The reaction was stirred at 25° C. for 16 h. The mixture was diluted with water, extracted with EtOAc, washed with brine, dried with Na₂SO₄, filtered and concentrated. The mixture was purified by recrystallization to give title compound (15.4 g). LCMS (ESI): m/z, 377.3, 378.1 [M+H]⁺.

2. Synthesis of 2-chloro-5-(2,5-dihydrofuran-2-yl)-N-(4-phenoxyphenyl)pyrimidin-4-amine. To a solution of 5-bromo-2-chloro-N-(4-phenoxyphenyl)pyrimidin-4-amine (0.2 g, 0.53 mmol) 2,3-dihydrofuran (55.8 mg, 0.80 mmol) in dioxane (3 mL) and H₂O (1 mL) was added K₂CO₃ (184 mg, 1.33 mmol) and Pd(dppf)Cl₂ (38.9 mg, 0.05 mmol) at 25° C. The reaction mixture was purged by N₂ and stirred at 90° C. for 12 h. The reaction mixture was quenched by sat. NH₄Cl and extracted with EtOAc. The combined organic phase was washed with brine, dried over anhydrous Na₂SO₄, filtered and concentrated in vacuum to give a residue which was purified by column chromatography to afford title compound (40 mg). LCMS (ESI): m/z, 366.1, 368.1 [M+H]⁺.

3. Synthesis of 2-chloro-N-(4-phenoxyphenyl)-5-(tetrahydrofuran-2 yl)pyrimidin-4-amine. To a solution of 2-chloro-5-(2,5-dihydrofuran-2-yl)-N-(4-phenoxyphenyl)pyrimidin-4-amine (40.0 mg, 0.11 mmol) in EtOH (2 mL) was added PtO₂ (2.5 mg, 0.01 mmol) at 25° C. The reaction mixture was stirred at 25° C. for 12 h under H₂ (15 psi) atmosphere. The reaction mixture was filtered by diatomite and washed with (DCM:MeOH=10:1, 30 mL). The filtrate was concentrated in vacuum to give a residue which was purified by prep-TLC to give title compound (30 mg). ¹H NMR (400 MHz, CDCl₃) δ 8.68 (s, 1H), 7.95 (s, 1H), 7.59-7.52 (m, 2H), 7.37-7.30 (m, 2H), 7.09 (t, J=7.6 Hz, 1H), 7.06-6.97 (m, 4H), 4.85-4.78 (m, 1H), 4.12 (dt, J=6.8, 8.4 Hz, 1H), 4.04-3.97 (m, 1H), 2.36-2.28 (m, 1H), 2.17-2.03 (m, 3H); LCMS (EST): m/z, 368.1, 370.1 [M+H]⁺.

4. Synthesis of N-(4-phenoxyphenyl)-5-(tetrahydrofuran-2-yl)-2-vinylpyrimidin-4-amine. To a solution of 2-chloro-N-(4-phenoxyphenyl)-5-tetrahydrofuran-2-yl-pyrimidin-4-amine (350 mg, 0.95 mmol), potassium trifluoro(vinyl)boranuide (191 mg, 1.43 mmol) in dioxane (7 mL) and H₂O (2 mL) were added K₂CO₃ (329 mg, 2.38 mmol) and Pd(dppf)Cl₂ (139 mg, 0.19 mmol) at 25° C. The reaction mixture was purged by N₂ and stirred at 90° C. for 12 h. The reaction mixture was quenched by sat. NH₄Cl and extracted with EA. The combined organic phase was washed with brine, dried over anhydrous Na₂SO₄, filtered and concentrated in vacuum to give a residue which was purified by column chromatography to afford title compound (0.23 g). LCMS (ESI): m/z, 360.2 [M+H]⁺.

5. Synthesis of 4-((4-phenoxyphenyl)amino)-5-(tetrahydrofuran-2-yl)pyrimidine-2-carbaldehyde. To a solution of N-(4-phenoxyphenyl)-5-tetrahydrofuran-2-yl-2-vinyl-pyrimidin-4-amine (0.2 g, 0.56 mmol) in THE (6 mL) and H₂O (1.2 mL) was added NaIO₄ (300 mg, 1.39 mmol) and potassium tetraoxoosmium(2-) hydrate (17 mg, 0.056 mmol) at 25° C. The reaction mixture was stirred at 25° C. for 12 h. The reaction mixture was filtered and washed with EA. The filtrate was diluted with water and extracted with EA. The combined organic phases were washed with brine), dried over anhydrous Na₂SO₄, filtered and concentrated to give title compound (0.21 g). LCMS (ESI): 380.2 [M+H+H₂O]⁺.

6. Synthesis of (4-((4-phenoxyphenyl)amino)-5-(tetrahydrofuran-2-yl)pyrimidin-2-yl)methanol. To a solution of 4-(4-phenoxyanilino)-5-tetrahydrofuran-2-yl-pyrimidine-2-carbaldehyde (210 mg, 0.58 mmol) in THE (8 mL) was added sodium triacetoxyboranuide (370 mg, 1.74 mmol) at 25° C. The reaction mixture was stirred at 25° C. for 12 h. The reaction mixture was quenched by sat. NH₄Cl and extracted with EA. The combined organic phase was washed with brine (10 mL), dried over anhydrous Na₂SO₄, filtered and concentrated to give a residue which was purified by column chromatography to give title compound (40 mg). ¹H NMR (400 MHz, CDCl₃) δ 8.52 (s, 1H), 8.12 (s, 1H), 7.52 (d, J 8.8 Hz, 2H), 7.38-7.31 (m, 2H), 7.10 (m, 1H), 7.05-7.00 (m, 4H), 4.87-4.81 (m, 1H), 4.65 (s, 2H), 4.14 (m, 1H), 4.05-3.97 (m, 1H), 2.33 (m, 1H), 2.20-2.11 (m, 3H). LCMS (ESI): m/z, 364.2 [M+H]⁺.

7. Synthesis of E1 19. To a solution of (4-(4-phenoxyanilino)-5-tetrahydrofuran-2-yl-pyrimidin-2-yl]methanol (40 mg, 0.11 mmol) and 2-(trifluoromethyl)piperidine-1-carbonitrile (39.2 mg, 0.22 mmol) in anhydrous 1,4-dioxane (3 mL) was added dichlorozinc (45.0 mg, 0.33 mmol) at 25° C. The mixture was stirred at 90° C. for 6 h. The reaction mixture was concentrated to give a residue which was purified by prep-HPLC to give title compound (10 mg). ¹H NMR (400 MHz, CDCl₃) δ 8.51 (br s, 1H), 8.13 (s, 1H), 7.61 (br d, J=8.8 Hz, 2H), 7.37-7.31 (m, 2H), 7.10 (m, 1H), 7.04-6.96 (m, 4H), 5.21 (br d, J=13.2 Hz, 1H), 5.11-5.02 (m, 1H), 4.88-4.79 (m, 2H), 4.14 (m, 1H), 4.05-3.99 (m, 2H), 3.04 (m, 1H), 2.34-2.27 (m, 1H), 2.17-2.09 (m, 3H), 1.96 (m, 1H), 1.75-1.62 (m, 5H), 1.51-1.43 (m, 1H). LCMS (ESI): m/z, 542.3 [M+H]⁺.

### Example 184 Preparation of Compound E120

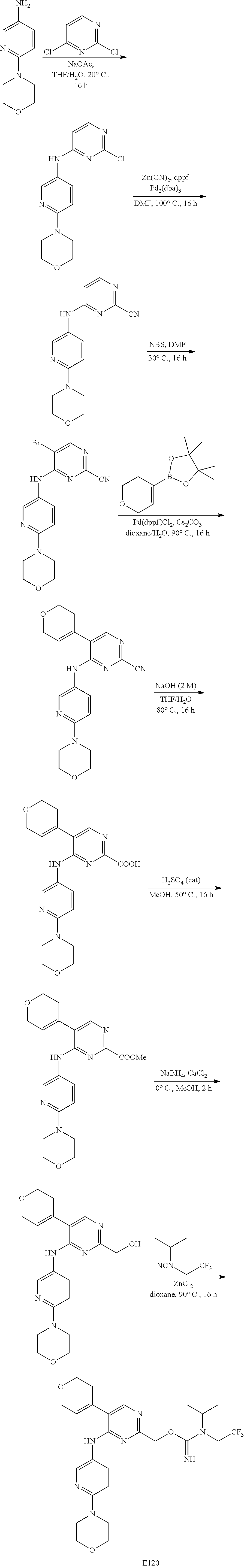

1. Synthesis of 2-chloro-N-(6-morpholinopyridin-3-yl)pyrimidin-4-amine. A mixture of 2,4-dichloropyrimidine (1.0 g, 6.71 mmol) and 6-morpholinopyridin-3-amine (1.0 g, 5.58 mmol) in THF (5 mL) and H₂O (5 mL) was added NaOAc (1.52 g, 11.16 mmol). The mixture was stirred at 20° C. for 16 h. The reaction mixture was quenched by saturated aqueous NH₄Cl, extracted with EA. The mixture was concentrated. The residue was purified by column chromatography to afford title compound (825 mg). LCMS (ESI): 292.1 [M+H]⁺.

2. Synthesis of 4-((6-morpholinopyridin-3-yl)amino)pyrimidine-2-carbonitrile. To a mixture of 2-chloro-N-(6-morpholinopyridin-3-yl)pyrimidin-4-amine (825 mg, 2.83 mmol), Zn(CN)₂ (996 mg, 8.48 mmol) and dppf (157 mg, 0.28 mmol) in DMF (1 mL) was added Pd₂(dba)₃ (259 mg, 0.28 mmol). The mixture was stirred at 100° C. for 16 h under N₂. The reaction mixture was quenched by aq. sat. NH₄Cl, extracted with EA. The mixture was concentrated. The residue was purified by column chromatography to afford title compound (530 mg). LCMS (ESI): m/z, 283.1 [M+H]⁺.

3. Synthesis of 5-bromo-4-((6-morpholinopyridin-3-yl)amino)pyrimidine-2-carbonitrile. To a mixture of 4-((6-morpholinopyridin-3-yl)amino)pyrimidine-2-carbonitrile (475 mg, 1.68 mmol) in DMF (10 mL) was added NBS (359 mg, 2.02 mmol). The mixture was stirred at 30° C. for 16 h. The reaction mixture was quenched by sat. NH₄Cl and extracted with EA. The combined organic phases were washed with brine, dried over anhydrous Na₂SO₄, filtered and concentrated. The mixture was concentrated. The residue was purified by column chromatography to afford title compound (110 mg). LCMS (ESI): m/z, 362.9 [M+H]⁺.

4. Synthesis of 5-(3,6-dihydro-2H-pyran-4-yl)-4-((6-morpholinopyridin-3-yl)amino)pyrimidine-2-carbonitrile. To a solution of 5-bromo-4-((6-morpholinopyridin-3-yl)amino)pyrimidine-2-carbonitrile (100 mg, 0.28 mmol) and 2-(3,6-dihydro-2H-pyran-4-yl)-4,4,5,5-tetramethyl-1,3,2-dioxaborolane (87 mg, 0.42 mmol) in 1,4-dioxane (2 mL) and H₂O (0.5 mL) was added Pd(dppf)Cl₂ (20 mg, 0.028 mmol) and Cs₂CO₃ (180 mg, 0.55 mmol). The mixture was stirred at 90° C. for 16 h under N₂. The reaction mixture was quenched by saturated aqueous NH₄Cl, extracted with EtOAc. The combined organic phases were washed with brine, dried over anhydrous Na₂SO₄, filtered and concentrated. The residue was purified by column chromatography to afford title compound (79 mg). LCMS (ESI): m/z, 365.1 [M+H]⁺.

5. Synthesis of 5-(3,6-dihydro-2H-pyran-4-yl)-4-((6-morpholinopyridin-3-yl)amino)pyrimidine-2-carboxylic acid. 5-(3,6-dihydro-2H-pyran-4-yl)-4-((6-morpholinopyridin-3-yl)amino)pyrimidine-2-carbonitrile (79 mg, 0.22 mmol) was added to the mixture of 2 M NaOH aq. (2 mL) and THF (0.5 mL). The mixture was stirred at 80° C. for 16 h. The reaction mixture was concentrated under vacuum to give title compound (83 mg). LCMS (ESI): m/z, 384.2 [M+H]⁺.

6. Synthesis of methyl 5-(3,6-dihydro-2H-pyran-4-yl)-4-((6-morpholinopyridin-3-yl)amino)pyrimidine-2-carboxylate. To a mixture of 5-(3,6-dihydro-2H-pyran-4-yl)-4-((6-morpholinopyridin-3-yl)amino)pyrimidine-2-carboxylic acid (83 mg, 0.22 mmol) in MeOH (1.5 mL) was added H₂SO₄ (2 mL, Con.). The mixture was stirred at 50° C. for 16 h. The mixture was adjusted pH=9 with aq. sat. NaHCO₃ and extracted with EA. The combined organic phases were washed with brine, dried over anhydrous Na₂SO₄, filtered and concentrated under vacuum. The residue was purified by column chromatography to afford title compound (67 mg). LCMS (ESI): m/z, 398.2 [M+H]⁺.

7. Synthesis of (5-(3,6-dihydro-2H-pyran-4-yl)-4-((6-morpholinopyridin-3-yl)amino)pyrimidin-2-yl)methanol. To a mixture of methyl 5-(3,6-dihydro-2H-pyran-4-yl)-4-((6-morpholinopyridin-3-yl)amino)pyrimidine-2-carboxylate (50 mg, 0.13 mmol) in MeOH (2 mL) was added NaBH₄ (9 mg, 0.25 mmol) and CaCl₂ (8 mg, 0.08 mmol) at 0° C. The mixture was stirred at 0° C. for 2 h. The reaction mixture was quenched by aq. sat. NH₄Cl, extracted with EA. The combined organic phases were washed with brine, dried over anhydrous Na₂SO₄, filtered and concentrated to give title compound (46 mg). LCMS (ESI): m/z, 370.2 [M+H]⁺. [001041]8. Synthesis of compound E120. To a mixture of (5-(3,6-dihydro-2H-pyran-4-yl)-4-((6-morpholinopyridin-3-yl)amino)pyrimidin-2-yl)methanol (50 mg, 0.14 mmol) and N-isopropyl-N-(2,2,2-trifluoroethyl)cyanamide (90 mg, 0.54 mmol) in 1,4-dioxane (2 mL) was added ZnCl₂ (73 mg, 0.54 mmol). The mixture was stirred at 90° C. for 16 h under N₂. The reaction mixture was quenched by saturated aqueous NH₄Cl (10 mL), extracted with EA. The combined organic phases were washed with brine, dried over anhydrous Na₂SO₄, filtered and concentrated. The crude was purified by prep-HPLC to afford title compound (11.0 mg). ¹H NMR (400 MHz, CDCl₃) δ 8.33-8.21 (m, 2H), 7.41-7.32 (m, 1H), 6.79-6.65 (m, 1H), 6.47 (d, J=6.0 Hz, 1H), 5.99-5.89 (m, 1H), 5.21-5.10 (m, 2H), 4.37-4.26 (m, 2H), 4.25-4.11 (m, 1H), 3.99-3.87 (m, 4H), 3.85-3.79 (m, 4H), 3.35-3.26 (m, 4H), 2.57 (d, J=1.2 Hz, 2H), 1.26-1.22 (m, 6H); LCMS (ESI): m/z, 536.3 [M+H]⁺.

### Example 185. Preparation of Compound E121

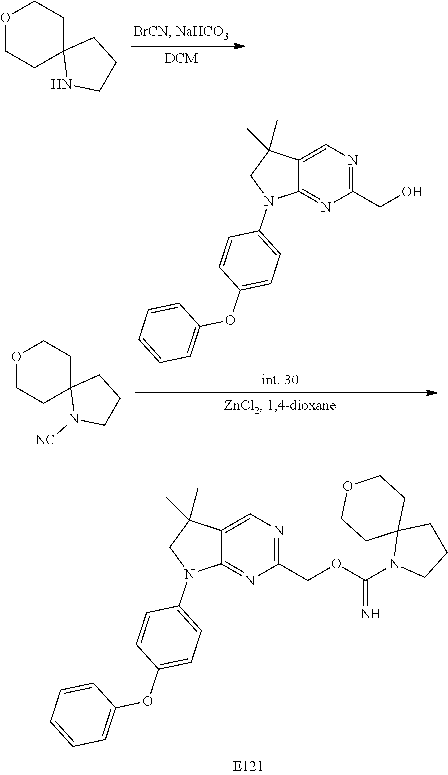 [SMILES: C1CNC2(C1)CCOCC2 ; N#CN1CCCC12CCOCC2 ; CC1(C)CN(c2ccc(Oc3ccccc3)cc2)c2nc(CO)ncc21 ; CC1(C)CN(c2ccc(Oc3ccccc3)cc2)c2nc(COC(=N)N3CCCC34CCOCC4)ncc21]

1. Synthesis of 8-oxa-1-azaspiro[4.5]decane-1-carbonitrile. To a solution of 8-oxa-1-azaspiro[4.5]decane (50 mg, 0.35 mmol) in DCM (1 mL) were added BrCN (56 mg, 0.53 mmol) and aq. sat. NaHCO₃ (0.5 mL). The mixture was stirred at 25° C. for 16 h. Water (10 mL) was added and extracted with DCM. The combined organic phases were washed with brine (20 mL), dried over anhydrous Na₂SO₄, filtered and concentrated to yield title compound (60 mg crude). ¹H NMR (400 MHz, CDCl₃) δ 4.08-3.93 (m, 2H), 3.54-3.36 (m, 4H), 2.08-2.01 (m, 2H), 1.99-1.84 (m, 4H), 1.55-1.47 (m, 2H); LCMS (EST): m/z, 167.1 [M+H]⁺.

2. Synthesis of E121. To a solution of [5,5-dimethyl-7-(4-phenoxyphenyl)-6H-pyrrolo[2,3-d]pyrimidin-2-yl]methanol (130 mg, 0.37 mmol) and 8-oxa-1-azaspiro[4.5]decane-1-carbonitrile (124 mg, 0.75 mmol) in 1,4-dioxane (5 mL) was added ZnCl₂ (204 mg, 1.50 mmol). The mixture was stirred at 100° C. for 16 h under N₂. The solvent was removed to yield a residue which was purified by prep-HPLC to give title compound (16.5 mg). ¹H NMR (400 MHz, CDCl₃) δ 8.06 (s, 1H), 7.85-7.77 (m, 2H), 7.37-7.28 (m, 2H), 7.13-7.05 (m, 1H), 7.03-6.97 (m, 4H), 5.27 (s, 2H), 3.86 (dd, J=5.2, 11.6 Hz, 2H), 3.80 (s, 2H), 3.48 (t, J=6.8 Hz, 2H), 3.44-3.34 (m, 2H), 2.88-2.80 (m, 2H), 2.03-1.98 (m, 2H), 1.83 (q, J=6.8 Hz, 2H), 1.43 (s, 6H), 1.30-1.23 (m, 2H); LCMS (ESI): m/z, 514.0 [M+H]⁺.

### Example 186. Preparation of Compound E122

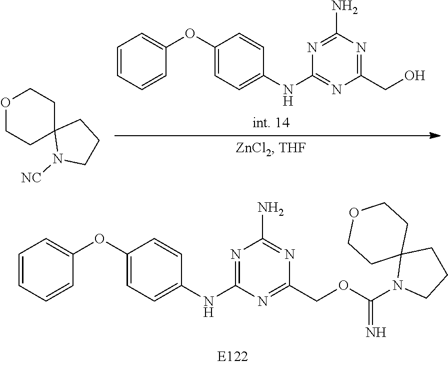 [SMILES: N#CN1CCCC12CCOCC2 ; Nc1nc(CO)nc(Nc2ccc(Oc3ccccc3)cc2)n1 ; N=C(OCc1nc(N)nc(Nc2ccc(Oc3ccccc3)cc2)n1)N1CCCC12CCOCC2]

To a solution of [4-amino-6-(4-phenoxyanilino)-1,3,5-triazin-2-yl]methanol (50 mg, 0.16 mmol) and 8-oxa-1-azaspiro[4.5]decane-1-carbonitrile (40 mg, 0.24 mmol) in 1,4-dioxane (4 mL) was added ZnCl₂ (88 mg, 0.65 mmol) and the mixture was stirred at 100° C. for 16 h under N₂. The solvent was removed to yield a residue which was purified by prep-HPLC to give title compound (8.3 mg). ¹H NMR (400 MHz, CDCl₃) δ 7.57 (d, J=8.4 Hz, 2H), 7.34-7.30 (m, 2H), 7.10-7.06 (m, 1H), 6.99 (dd, J=3.6, 8.8 Hz, 4H), 5.33 (s, 2H), 5.05 (s, 2H), 4.03-4.00 (m, 2H), 3.52-3.45 (m, 4H), 3.12 (s, 2H), 2.06-2.04 (m, 4H), 1.91-1.88 (m, 4H); LCMS (ESI): m/z, 476.3 [M+H]⁺.

### Example 187. Preparation of Compound E123

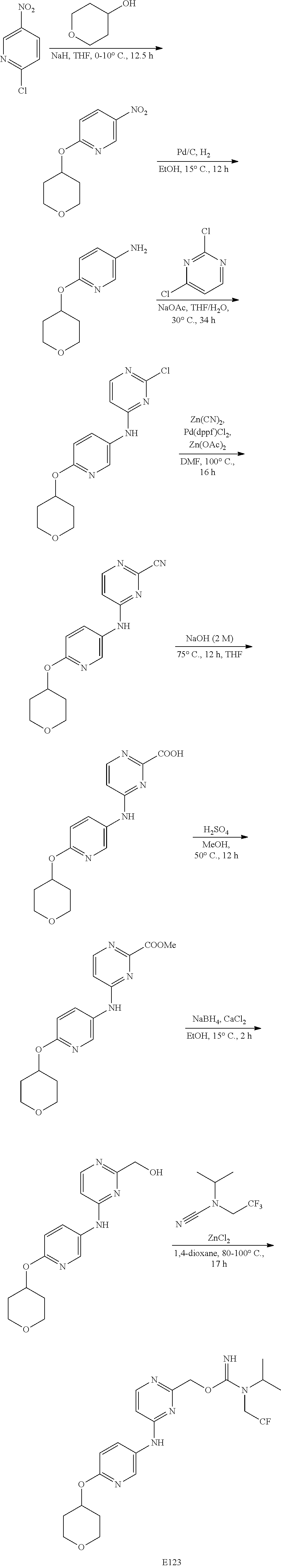

1. Synthesis of 5-nitro-2-((tetrahydro-2H-pyran-4-yl)oxy)pyridine. To a solution of tetrahydropyran-4-ol (10.0 g, 97.91 mmol) in THF (100 mL) was added NaH (5.87 g, 147 mmol, 60% in mineral oil) at 0° C., and the mixture was stirred at 0° C. for 30 min. To the mixture was added 2-chloro-5-nitro-pyridine (15.5 g, 97.9 mmol) and the mixture was stirred at 10° C. for 12 h. The mixture was concentrated. To the mixture was added H₂O and EtOAc, the aqueous phase was extracted with EtOAc. The combined organic layers were washed with brine, dried over Na₂SO₄, filtered and concentrated. The residue was purified by column chromatography to afford title compound (11.3 g).

2. Synthesis of 6-((Tetrahydro-2H-pyran-4-yl)oxy)pyridin-3-amine. To a solution of 5-nitro-2-((tetrahydro-2H-pyran-4-yl)oxy)pyridine (2.0 g, 8.92 mmol) in EtOH (40 mL) was added Pd/C (800 mg, 10%), the mixture was stirred at 15° C. for 12 h under H₂. The mixture was filtered and concentrated to give title compound (1.67 g).

3. Synthesis of 2-chloro-N-(6-((tetrahydro-2H-pyran-4-yl)oxy)pyridin-3-yl)pyrimidin-4-amine. To a mixture of 6-((tetrahydro-2H-pyran-4-yl)oxy)pyridin-3-amine (1.67 g, 8.60 mmol) and NaOAc (1.41 g, 17.20 mmol) in THF (15 mL) and H₂O (10 mL) was added 2,4-dichloropyrimidine (1.28 g, 8.60 mmol) and the mixture was heated at 30° C. for 34 h. To the mixture was added EtOAc, the aqueous phase was extracted with EtOAc. The combined organic layers were washed with brine and dried over Na₂SO₄, filtered and concentrated. The residue was purified by column chromatography to afford title compound (1.11 g).

4. Synthesis of 4-((6-((tetrahydro-2H-pyran-4-yl)oxy)pyridin-3-yl)amino)pyrimidine-2-carbonitrile. To a solution of 2-chloro-N-(6-((tetrahydro-2H-pyran-4-yl)oxy)pyridin-3-yl)pyrimidin-4-amine (500 mg, 1.63 mmol), Zn(CN)₂ (115 mg, 0.98 mmol) and Pd(dppf)Cl₂ (119 mg, 0.16 mmol) in DMF (10 mL) was added Zn(OAc)₂ (30 mg, 0.16 mmol), the mixture was heated at 100° C. for 16 h under N₂. The mixture was concentrated. The crude product was purified by column chromatography to afford title compound (330 mg).

5. Synthesis of 5-(3,6-dihydro-2H-pyran-4-yl)-4-((6-morpholinopyridin-3-yl)amino)pyrimidine-2-carboxylic acid. A solution of 4-[(6-tetrahydropyran-4-yloxy-3-pyridyl)amino]pyrimidine-2-carbonitrile (500 mg, 1.68 mmol) in NaOH (2 M, 4 mL) and THF (1 mL) was heated at 75° C. for 12 h. The mixture was adjusted pH to 6 with HCl (2M) and concentrated to give title compound (532 mg).

6. Synthesis of methyl 4-((6-((tetrahydro-2H-pyran-4-yl)oxy)pyridin-3-yl)amino)pyrimidine-2-carboxylate. To a solution of 4-((6-((tetrahydro-2H-pyran-4-yl)oxy)pyridin-3-yl)amino)pyrimidine-2-carboxylic acid (480 mg, 1.52 mmol) in MeOH (10 mL) was added conc. H₂SO₄ (0.5 mL), the mixture was heated at 50° C. for 12 h. The mixture was adjusted to pH>7 with sat. aq. NaHCO₃ and concentrated. To the residue was added H₂O and EA, the aqueous phase was extracted with EA. The combined organic layers were washed with brine and dried over Na₂SO₄, filtered and concentrated. The crude product was purified by column chromatography to give title compound (220 mg).

7. Synthesis of (4-((6-((tetrahydro-2H-pyran-4-yl)oxy)pyridin-3-yl)amino)pyrimidin-2-yl)methanol. To a solution of methyl 4-((6-((tetrahydro-2H-pyran-4-yl)oxy)pyridin-3-yl)amino)pyrimidine-2-carboxylate (200 mg, 0.61 mmol) in EtOH (5 mL) was added NaBH₄ (69 mg, 1.82 mmol) and the mixture was stirred at 15° C. for 2 h. To the mixture was added H₂O and EA, the aqueous phase was extracted with EA. The combined organic layers were washed with brine and dried over Na₂SO₄, filtered and concentrated to give title compound (190 mg).

8. Synthesis of compound E123. To a solution of (4-((6-((tetrahydro-2H-pyran-4-yl)oxy)pyridin-3-yl)amino)pyrimidin-2-yl)methanol (90 mg, 0.30 mmol) and N-isopropyl-N-(2,2,2-trifluoroethyl)cyanamide (99 mg, 0.60 mmol) in 1,4-dioxane (2 mL) was added ZnCl₂ (162 mg, 1.19 mmol) and the mixture was heated at 80° C. for 12 h under N₂. To the mixture was added N-isopropyl-N-(2,2,2-trifluoroethyl)cyanamide (99 mg, 0.60 mmol) and ZnCl₂ (81 mg, 0.60 mmol) and the mixture was heated at 100° C. for 2 h. To the mixture was added ZnCl₂ (81 mg, 0.60 mmol), the mixture was heated at 100° C. for 3 h under N₂. The residue was concentrated and the crude was purified by preparative-HPLC to give title compound (30.0 mg). ¹H NMR (400 MHz, CDCl₃) δ 8.26 (d, J=6.0 Hz, 1H), 8.15 (d, J=2.8 Hz, 1H), 7.61-7.67 (m, 1H), 6.77 (d, J=8.8 Hz, 1H), 6.70 (s, 1H), 6.40 (d, J=6.0 Hz, 1H), 5.18-5.26 (m, 1H), 5.16 (s, 2H), 4.15-4.24 (m, 1H), 3.85-4.07 (m, 5H), 3.56-3.67 (m, 2H), 2.04-2.12 (m, 2H), 1.76-1.86 (m, 2H), 1.24 (d, J=6.8 Hz, 6H). LCMS (ESI): m/z, 469.2 [M+H]⁺.

### Example 188. Preparation of Compound E124

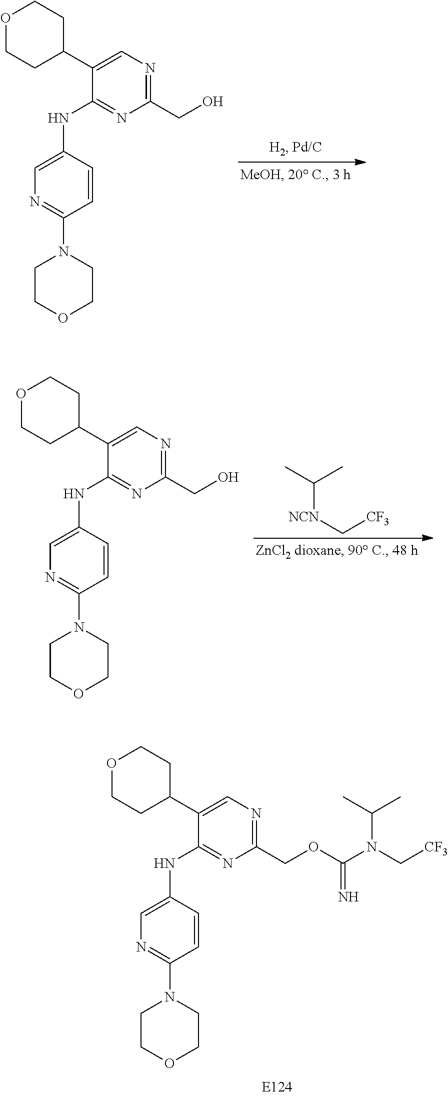

1. Synthesis of (4-((6-morpholinopyridin-3-yl)amino)-5-(tetrahydro-2H-pyran-4-yl)pyrimidin-2-yl)methanol. To a solution of (5-(3,6-dihydro-2H-pyran-4-yl)-4-((6-morpholinopyridin-3-yl)amino)pyrimidin-2-yl)methanol (85 mg, 0.23 mmol) in MeOH (20 mL) was added Pd/C (10%, 34 mg) under N₂. The suspension was degassed under vacuum and purged with H₂ 3 times. The mixture was stirred under H₂ balloon at 20° C. for 3 h. The mixture was filtered and filter cake was washed with MeOH. The filtrate was concentrated. The residue was purified by column chromatography to give title compound (70 mg). ¹H NMR (400 MHz, CDCl₃) δ 8.22 (d, J=2.8 Hz, 1H), 8.19 (s, 1H), 7.65-7.74 (m, 1H), 6.64-6.74 (m, 1H), 6.31-6.46 (m, 1H), 4.54-4.61 (m, 2H), 4.11-4.22 (m, 2H), 3.81-3.92 (m, 4H), 3.56-3.66 (m, 2H), 3.43-3.55 (m, 5H), 2.66-2.80 (m, 1H), 1.84-1.97 (m, 4H); LCMS (ESI): m/z, 372.1 [M+H]⁺.

2. Synthesis of compound E124. To a mixture of (4-((6-morpholinopyridin-3-yl)amino)-5-(tetrahydro-2H-pyran-4-yl)pyrimidin-2-yl)methanol (70 mg, 0.19 mmol) and N-isopropyl-N-(2,2,2-trifluoroethyl)cyanamide (125 mg, 0.75 mmol) in dioxane (2 mL) was added ZnCl₂ (102 mg, 0.75 mmol). The mixture was stirred at 90° C. for 48 h under N₂. The reaction mixture was quenched by saturated aqueous NH₄Cl, extracted with EA. The combined organic phases were washed with brine, dried over anhydrous Na₂SO₄, filtered and concentrated. The crude was purified by prep-HPLC to give title compound (11.0 mg).

¹H NMR (400 MHz, CDCl₃) δ 8.22-8.25 (m, 1H), 8.16-8.20 (m, 1H), 7.69-7.76 (m, 1H), 6.66-6.75 (m, 1H), 6.39-6.52 (m, 1H), 5.12-5.21 (m, 2H), 4.22-4.36 (m, 1H), 4.12-4.20 (m, 2H), 3.87-3.96 (m, 2H), 3.83-3.87 (m, 4H), 3.58-3.65 (m, 2H), 3.49-3.53 (m, 4H), 2.69-2.78 (m, 1H), 1.87-1.92 (m, 4H), 1.20 (d, J=6.8 Hz, 6H). LCMS (ESI): m/z, 536.3 [M+H]⁺.

### Example 189. Preparation of Compound E125

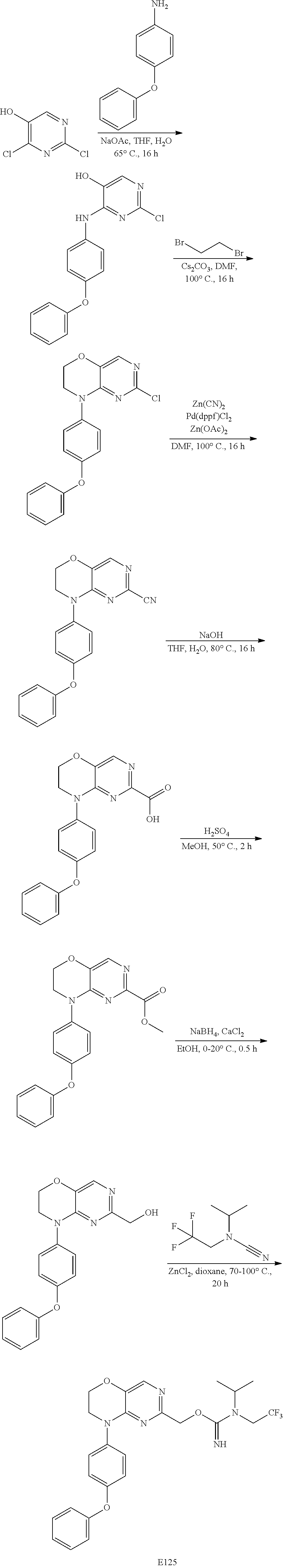

1. Synthesis of 2-chloro-4-((4-phenoxyphenyl)amino)pyrimidin-5-ol. To a solution of 2,4-dichloropyrimidin-5-ol (2.02 g, 12.27 mmol) and 4-phenoxyaniline (2.5 g, 13.50 mmol) in THF (10 mL) and H₂O (10 mL) was added NaOAc (2.01 g, 24.54 mmol) and stirred at 65° C. for 16 h. The reaction was added water (100 mL) and extracted with EA. The organic layers were washed with brine, dried over Na₂SO₄, filtered and concentrated to give a residue which was purified by column chromatography to give title compound (2.33 g). ¹H NMR (400 MHz, DMSO-d₆) δ 10.75 (s, 1H), 9.18 (s, 1H), 7.82-7.74 (m, 2H), 7.71 (s, 1H), 7.42-7.32 (m, 2H), 7.15-7.07 (m, 1H), 7.05-6.95 (m, 4H).

2. Synthesis of 2-chloro-8-(4-phenoxyphenyl)-7,8-dihydro-6H-pyrimido[5,4-b][1,4]oxazine. To a solution of 2-chloro-4-((4-phenoxyphenyl)amino)pyrimidin-5-ol (593 mg, 1.89 mmol) and 1,2-dibromoethane (355 mg, 1.89 mmol) in DMF (1 mL) was added Cs₂CO₃ (1.23 g, 3.78 mmol) and stirred at 100° C. for 16 h. The reaction was added water and extracted with EA. The organic layers were washed with brine, dried over Na₂SO₄, filtered and concentrated to give a residue which was purified by column chromatography to give title compound (560 mg).

3. Synthesis of 8-(4-phenoxyphenyl)-7,8-dihydro-6H-pyrimido[5,4-b][1,4]oxazine-2-carbonitrile. To a solution of 2-chloro-8-(4-phenoxyphenyl)-7,8-dihydro-6H-pyrimido[5,4-b][1,4]oxazine (0.3 g, 0.88 mmol) in DMF (5 mL) were added Pd(dppf)Cl₂ (129 mg, 0.18 mmol), Zn(CN)₂ (62 mg, 0.53 mmol) and Zn(OAc)₂ (16 mg, 0.088 mmol) and stirred at 100° C. for 16 h. The reaction was filtered and concentrated give a residue which was purified by column chromatography to give title compound (126 mg).

4. Synthesis of 8-(4-phenoxyphenyl)-7,8-dihydro-6H-pyrimido[5,4-b][1,4]oxazine-2-carboxylic acid. To a solution of 8-(4-phenoxyphenyl)-7,8-dihydro-6H-pyrimido[5,4-b][1,4]oxazine-2-carbonitrile (126 mg, 0.38 mmol) in NaOH (4 mL, 2 M) and THF (1 mL) and stirred at 80° C. for 16 h. After the reaction mixture was cooled to 0° C., the reaction mixture was quenched by addition of 6 mL of 2M HCL. The reaction was concentrated to afford title compound (134 mg).

5. Synthesis of methyl 8-(4-phenoxyphenyl)-7,8-dihydro-6H-pyrimido[5,4-b][1,4]oxazine-2-carboxylate. To a solution of 8-(4-phenoxyphenyl)-7,8-dihydro-6H-pyrimido[5,4-b][1,4]oxazine-2-carboxylic acid (134 mg, 0.38 mmol) in MeOH (5 mL) was added dropwise H₂SO₄ (1 mL) and stirred at 50° C. for 2 h. After the reaction mixture was cooled to 0° C., the reaction mixture was quenched by addition of 5 mL of H₂O. The reaction was added NaHCO₃ and extracted with EA. The organic layers were washed with brine, dried over Na₂SO₄, filtered and concentrated to give a residue which was purified by column chromatography to give title compound (111 mg).

6. Synthesis of (8-(4-phenoxyphenyl)-7,8-dihydro-6H-pyrimido[5,4-b][1,4]oxazin-2-yl)methanol. To a solution of methyl 8-(4-phenoxyphenyl)-7,8-dihydro-6H-pyrimido[5,4-b][1,4]oxazine-2-carboxylate (111 mg, 0.31 mmol) and CaCl₂ (20 mg, 0.18 mmol) in EtOH (3 mL) was added NaBH₄ (46 mg, 1.22 mmol) at 0° C., the mixture was stirred at 20° C. for 0.5 h. The reaction was filtered and concentrated to give a residue which was purified by column chromatography to give title compound (16 mg).

7. Synthesis of compound E125. To a solution of (8-(4-phenoxyphenyl)-7,8-dihydro-6H-pyrimido[5,4-b][1,4]oxazin-2-yl)methanol (15 mg, 0.45 mmol) and N-isopropyl-N-(2,2,2-trifluoroethyl)cyanamide (22 mg, 0.13 mmol) in 1,4-dioxane was added ZnCl₂ (24 mg, 0.18 mmol) and stirred at 70° C. for 4 h under N₂. Then stirred at 100° C. for 16 h. The reaction was filtered and concentrated to give a residue which was purified by prep-HPLC to give title compound (11.0 mg). ¹H NMR (400 MHz, DMSO-d₆) δ 7.94 (s, 1H), 7.60-7.26 (m, 4H), 7.25-6.80 (m, 5H), 5.88 (d, J=53.2 Hz, 1H), 4.88 (s, 2H), 4.34 (s, 2H), 3.93 (s, 5H), 1.01 (d, J=6.8 Hz, 6H). LCMS (ESI): m/z, 502.1 [M+H]⁺.

### Example 190. Preparation of Compound E126

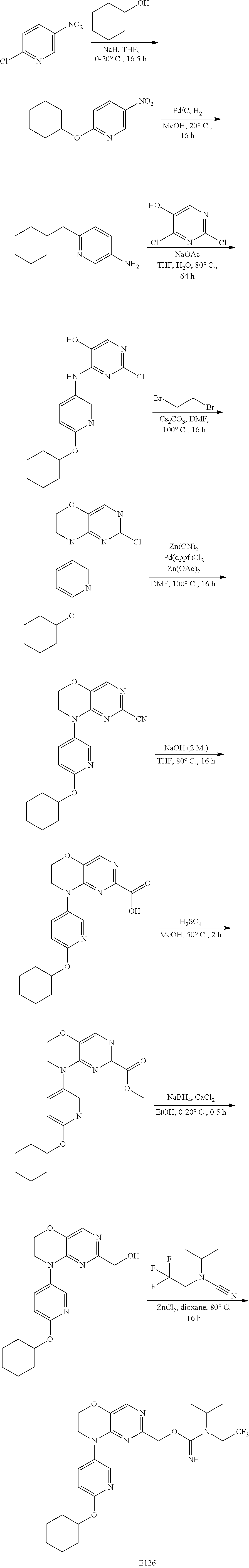

1. Synthesis of 2-(cyclohexyloxy)-5-nitropyridine. To a solution of cyclohexanol (0.5 g, 4.99 mmol) in THF (3 mL) was added NaH (300 mg, 7.49 mmol, 60% purity) at 0° C. and stirred at 20° C. for 0.5 h, then 2-chloro-5-nitropyridine (871 mg, 5.49 mmol) was added and stirred at 20° C. for 16 h. After the reaction mixture was cooled to 0° C., the mixture was quenched by addition of 50 mL of H₂O and extracted with EA. The organic layers were washed with brine, dried over Na₂SO₄, filtered and concentrated to give a residue which was purified by column chromatography to afford title compound (405 mg).

2. Synthesis of 6-(cyclohexyloxy)pyridin-3-amine. To a solution of 2-(cyclohexyloxy)-5-nitropyridine (400 mg, 1.80 mmol) in MeOH (10 mL) was added Pd/C (192 mg, 1.80 mmol) and stirred at 20° C. for 16 h under H₂. The reaction was filtered and concentrated to give title compound (345 mg). ¹H NMR (400 MHz, CDCl₃) δ 7.58 (d, J=3.2 Hz, 1H), 6.94 (dd, J=3.2, 8.4 Hz, 1H), 6.48 (d, J=8.4 Hz, 1H), 4.78 (td, J=9.2, 4.4 Hz, 1H), 3.26 (s, 2H), 1.92 (dt, J=3.6, 14.0 Hz, 2H), 1.73-1.64 (m, 2H), 1.51 (ddd, J=2.8, 6.0, 12.4 Hz, 1H), 1.42-1.18 (m, 5H).

3. Synthesis of 2-chloro-4-((6-(cyclohexyloxy)pyridin-3-yl)amino)pyrimidin-5-ol. To a solution of 2,4-dichloropyrimidin-5-ol (179 mg, 1.09 mmol) and 6-(cyclohexyloxy)pyridin-3-amine (230 mg, 1.20 mmol) in THF (2 mL) and H₂O (2 mL) was added NaOAc (178 mg, 2.18 mmol) and stirred at 80° C. for 64 h. The reaction was added water (20 mL) and extracted with EA. The organic layers were washed with brine, dried over Na₂SO₄, filtered and concentrated to give a residue which was purified by column chromatography to give title compound (160 mg).

4. Synthesis of 2-chloro-8-(6-(cyclohexyloxy)pyridin-3-yl)-7,8-dihydro-6H-pyrimido[5,4-b][1,4]oxazine. To a solution of 2-chloro-4-((6-(cyclohexyloxy)pyridin-3-yl)amino)pyrimidin-5-ol (160 mg, 0.50 mmol) and 1,2-dibromoethane (94 mg, 0.50 mmol) in DMF (1 mL) was added Cs₂CO₃ (325 mg, 1.00 mmol) and stirred at 100° C. for 16 h. The reaction was concentrated to give a residue which was purified by column chromatography to give title compound (106 mg).

5. Synthesis of 8-(6-(Cyclohexyloxy)pyridin-3-yl)-7,8-dihydro-6H-pyrimido[5,4-b][1,4]oxazine-2-carbonitrile. To a solution of 2-chloro-8-(6-(cyclohexyloxy)pyridin-3-yl)-7,8-dihydro-6H-pyrimido[5,4-b][1,4]oxazine (106 mg, 0.31 mmol) in DMF (2 mL) were added Pd(dppf)Cl₂ (45 mg, 0.06 mmol) and Zn(CN)₂ (22 mg, 0.18 mmol) and Zn(OAc)₂ (6 mg, 0.03 mmol). The mixture was stirred at 100 [symbol] C for 16 h under N₂. The reaction was filtered and concentrated to give a residue which was purified by column chromatography to give title compound (60 mg).

6. Synthesis of 8-(6-(cyclohexyloxy)pyridin-3-yl)-7,8-dihydro-6H-pyrimido[5,4-b][1,4]oxazine-2-carboxylic acid. To a solution of 8-(6-(cyclohexyloxy)pyridin-3-yl)-7,8-dihydro-6H-pyrimido[5,4-b][1,4]oxazine-2-carbonitrile (60 mg, 0.18 mmol) in NaOH (4 mL, 2 M) and THF (1 mL) and stirred at 80° C. for 16 h. After the reaction mixture was cooled to 0° C., the reaction mixture was quenched by addition of 5 mL of 2M HCl. The reaction was concentrated to give title compound (64 mg).

7. Synthesis of Methyl 8-(6-(cyclohexyloxy)pyridin-3-yl)-7,8-dihydro-6H-pyrimido[5,4-b][1,4]oxazine-2-carboxylate. To a solution of 8-(6-(cyclohexyloxy)pyridin-3-yl)-7,8-dihydro-6H-pyrimido[5,4-b][1,4]oxazine-2-carboxylic acid (64 mg, 0.18 mmol) in MeOH (5 mL) was added dropwise H₂SO₄ (1 mL) and stirred at 50° C. for 2 h. After the reaction mixture was cooled to 0° C., the reaction mixture was quenched by addition of 5 mL of H₂O. The reaction was added NaHCO₃ and extracted with EA. The organic layers were washed with brine, dried over Na₂SO₄, filtered and concentrated to give a residue which was purified by column chromatography to give title compound (45 mg).

8. Synthesis of (8-(6-(Cyclohexyloxy)pyridin-3-yl)-7,8-dihydro-6H-pyrimido[5,4-b][1,4]oxazin-2-yl)methanol. To a solution of methyl 8-(6-(cyclohexyloxy)pyridin-3-yl)-7,8-dihydro-6H-pyrimido[5,4-b][1,4]oxazine-2-carboxylate (45 mg, 0.12 mmol) and CaCl₂ (8 mg, 0.07 mmol) in EtOH (3 mL) was added NaBH₄ (18 mg, 0.49 mmol) at 0° C., the mixture was stirred at 20° C. for 0.5 h. After the reaction mixture was cooled to 0° C., the reaction mixture was quenched by addition of 5 mL of H₂O. The reaction was added water and extracted with EA. The organic layers were washed with brine, dried over Na₂SO₄, filtered and concentrated to give a residue which was purified by column chromatography to give title compound (35 mg).

9. Synthesis of compound E126. To a solution of (8-(6-(cyclohexyloxy)pyridin-3-yl)-7,8-dihydro-6H-pyrimido[5,4-b][1,4]oxazin-2-yl)methanol (35 mg, 0.10 mmol) and N-isopropyl-N-(2,2,2-trifluoroethyl)cyanamide (51 mg, 0.31 mmol) in 1,4-dioxane (1 mL) was added ZnCl₂ (56 mg, 0.41 mmol) and stirred at 70° C. for 16 h under N₂. The reaction was filtered and concentrated to give a residue which was purified by prep-HPLC separation to give title compound (15.0 mg). ¹H NMR (400 MHz, DMSO-d₆) δ 8.21 (d, J=2.8 Hz, 1H), 7.93 (s, 1H), 7.75 (dd, J=2.8, 8.8 Hz, 1H), 6.80 (d, J=8.8 Hz, 1H), 5.97 (s, 1H), 5.07-4.92 (m, 1H), 4.86 (s, 2H), 4.35 (t, J=4.4 Hz, 2H), 4.16-3.97 (m, 1H), 3.96-3.80 (m, 4H), 2.04-1.92 (m, 2H), 1.81-1.67 (m, 2H), 1.59-1.27 (m, 6H), 1.00 (d, J=6.8 Hz, 6H). LCMS (ESI): m/z, 509.2 [M+H]⁺.

### Example 191. Preparation of Compound E127

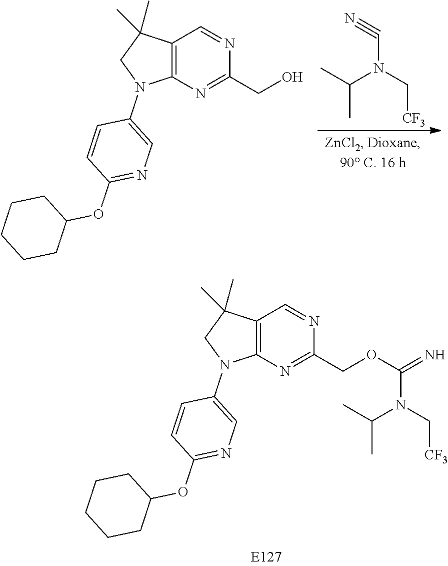

To a solution of [7-[6-(cyclohexoxy)-3-pyridyl]-5,5-dimethyl-6H-pyrrolo[2,3-d]pyrimidin-2-yl]methanol (40 mg, 0.11 mmol) and isopropyl(2,2,2-trifluoroethyl)cyanamide (56 mg, 0.34 mmol) was added dichlorozinc (6 mg, 0.45 mmol) in 1,4-dioxane (2 mL) at 10° C. The reaction was stirred at 90° C. for 16 h. The mixture was diluted with water (20 mL), extracted with EA, washed with brine, dried with Na₂SO₄, filtered and concentrated. The residue was purified by preparative HPLC to give title compound (5 mg). LCMS (ESI): 520.5, 521.3 [M+H]⁺.

### Example 192. Preparation of Compound E128

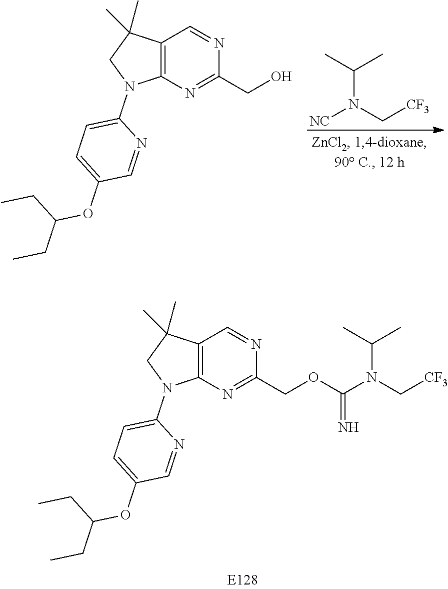 [SMILES: CCC(CC)Oc1ccc(N2CC(C)(C)c3cnc(CO)nc32)nc1 ; CC(C)N(C#N)CC(F)(F)F ; CCC(CC)Oc1ccc(N2CC(C)(C)c3cnc(COC(=N)N(CC(F)(F)F)C(C)C)nc32)nc1]

To a solution of [7-[5-(1-ethylpropoxy)-2-pyridyl]-5,5-dimethyl-6H-pyrrolo[2,3-d]pyrimidin-2-yl]methanol (25 mg, 0.073 mol) and isopropyl(2,2,2-trifluoroethyl)cyanamide (36 mg, 0.22 mol) in anhydrous 1,4-dioxane (1 mL) was added dichlorozinc (40 mg, 0.29 mol). The mixture was stirred at 90° C. for 12 h. The reaction mixture was concentrated to give a residue which was purified by prep-HPLC to give title compound (13 mg). LCMS (ESI): m/z, 509.3 [M+H]⁺.

### Example 193. Preparation of Compound E129

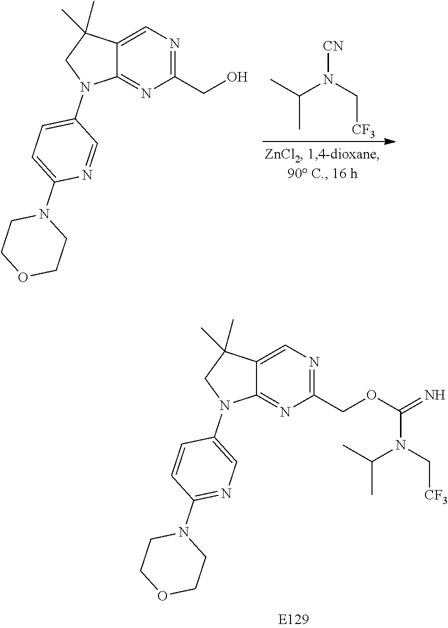

To a solution of (5,5-dimethyl-7-(6-morpholinopyridin-3-yl)-6,7-dihydro-5H-pyrrolo[2,3-d]pyrimidin-2-yl)methanol (17 mg, 0.05 mmol) and isopropyl(2,2,2-trifluoroethyl)cyanamide (25 mg, 149.38 mmol) in THE (2 mL) was added ZnCl₂ (27 mg, 0.2 mmol) and the mixture was stirred at 90° C. for 16 h under N₂. The solvent was removed to yield a residue which was purified by prep-HPLC. After prep. HPLC purification, the eluent was adjusted to pH 8 with sat. aq. NaHCO₃ and concentrated. The residual aqueous solution was extracted with EtOAc. The combined organic phases were concentrated to yield a residue which was dissolved in CAN and treated with water, followed by lyophilization to give title compound (3.3 mg). ¹H NMR (400 MHz, CDCl₃) δ 8.38 (d, J=2.4 Hz, 1H), 8.21 (dd, J=2.8, 9.2 Hz, 1H), 8.01 (s, 1H), 6.69 (d, J=9.2 Hz, 1H), 5.14 (s, 2H), 4.81 (br s, 3H), 4.23 (dt, J=6.8, 13.6 Hz, 1H), 3.94 (q, J=9.2 Hz, 2H), 3.88-3.81 (m, 4H), 3.76 (s, 2H), 3.56-3.39 (m, 4H), 1.43 (s, 6H), 1.22 (d, J=6.8 Hz, 6H); LCMS (ESI): m/z, 508.3 [M+H]⁺.

### Example 194. Preparation of Compound E130

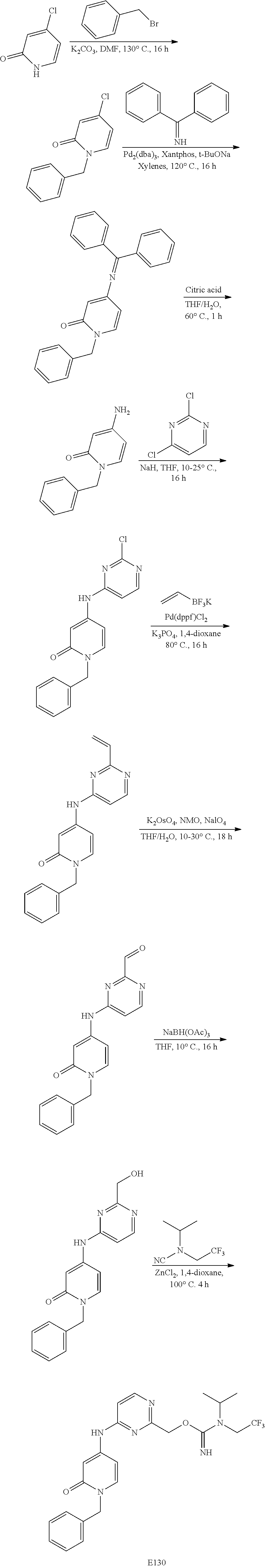

1. Synthesis of 1-benzyl-4-chloro-pyridin-2-one. A mixture of 4-chloro-1H-pyridin-2-one (7 g, 54.04 mmol), bromomethylbenzene (18.48 g, 108.07 mmol, 12.84 mL) and K₂CO₃ (14.94 g, 108.07 mmol) in DMF (200 mL) was stirred at 130° C. for 16 h. The solvent was removed and the residue was treated with EtOAc (200 mL). The mixture was filtered and the filtrate was concentrated to yield a residue which was purified by column chromatography to give title compound (9.0 g). ¹H NMR (400 MHz, CDCl₃) δ 7.38-7.25 (m, 5H), 7.19 (d, J=7.2 Hz, 1H), 6.66 (d, J=2.4 Hz, 1H), 6.17 (dd, J=2.4, 7.2 Hz, 1H), 5.10 (s, 2H).

2. Synthesis of 4-(benzhydrylideneamino)-1-benzyl-pyridin-2-one. To a solution of 1-benzyl-4-chloro-pyridin-2-one (3.0 g, 13.66 mmol) and diphenylmethanimine (3.71 g, 20.49 mmol) in Xylenes (180 mL) were added (5-diphenylphosphanyl-9,9-dimethyl-xanthen-4-yl)-diphenyl-phosphane (790 mg, 1.37 mmol), Pd₂(dba)₃ (625 mg, 0.68 mmol) and t-BuONa (2.63 g, 27.31 mmol). The mixture was stirred at 120° C. for 16 h under N₂. The solvent was removed and water was added, extracted with EtOAc. The combined organic phases were washed with brine, dried over anhydrous Na₂SO₄, filtered and concentrated to give title compound (5.0 g). LCMS (ESI): m/z, 365.1 [M+H]⁺.

3. Synthesis of 4-Amino-1-benzyl-pyridin-2-one. To a solution of 4-(benzhydrylideneamino)-1-benzyl-pyridin-2-one (5.0 g, 13.72 mmol) in THF (50 mL) and H₂O (10 mL) was added citric acid (5.27 g, 27.44 mmol). The mixture was stirred at 60° C. for 1 h. The solvent was removed to yield a residue which was purified by prep-HPLC to yield title compound (560 mg). ¹H NMR (400 MHz, CDCl₃) (7.36-7.26 (m, 5H), 7.03 (d, J=7.2 Hz, 1H), 5.69 (d, J=2.4 Hz, 1H), 5.63 (dd, J=2.4, 7.2 Hz, I H), 5.06 (s, 2H), 4.10 (s, 2H); LCMS (ESI): m/z, 201.1 [M+H]⁺.

4. Synthesis of 1-benzyl-4-[(2-chloropyrimidin-4-yl)amino]pyridin-2-one. To a solution of 4-amino-1-benzyl-pyridin-2-one (580 mg, 2.90 mmol) in THF (10 mL) was added NaH (232 mg, 5.79 mmol, 60% purity) at 10° C. and stirred at 10° C. for 10 min. 2,4-dichloropyrimidine (432 mg, 2.90 mmol) was added and the mixture was stirred at 25° C. for 16 h. The reaction was quenched by sat. aq. NH₄Cl, water was added and extracted with EtOAc. The combined organic phases were washed with brine, dried over anhydrous Na₂SO₄, filtered and concentrated. The residue was purified by prep-HPLC to yield title compound (290 mg). ¹H NMR (400 MHz, CD₃OD) δ 8.25 (d, J=6.0 Hz, 1H); 7.61 (d, J=7.6 Hz, 1H), 7.21-7.41 (m, 6H), 6.82 (d, J=5.6 Hz, 1H), 6.68 (dd, J=2.4, 7.6 Hz, 1H), (5.15 (s, 2H); LCMS (ESI): m/z, 313.1 [M+H]⁺.

5. Synthesis of 1-benzyl-4-[(2-vinylpyrimidin-4-yl)amino]pyridin-2-one. To a solution of 1-benzyl-4-[(2-chloropyrimidin-4-yl)amino]pyridin-2-one (290 mg, 0.93 mmol) and potassium trifluoro(vinyl)boranuide (621 mg, 4.64 mmol) in 1,4-dioxane (10 mL) were added Pd(dppf)Cl₂ (68 mg, 0.093 mmol) and K₃PO₄ (590 mg, 2.78 mmol). The mixture was stirred at 80° C. for 16 h under N₂. The solvent was removed to yield a residue which was treated with a solution of DCM/MeOH=10/1 (50 mL). The mixture was filtered and the filtrate was concentrated to yield a residue which was purified by column chromatography to give title compound (228 mg). ¹H NMR (400 MHz, CDCl₃) δ: 8.97 (br s, 1H), 8.22 (d, J=5.6 Hz, 1H), 7.37-7.26 (m, 5H), 7.14 (d, J=7.6 Hz, 1H), 6.98 (s, 1H), 6.80 (br d, J=7.2 Hz, 1H), 6.68 (dd, J=10.4, 17.2, 1H), 6.59-6.40 (m, 2H), 5.58 (dd, J=1.2, 10.4, 1H), 5.09 (s, 2H).

6. Synthesis of 4-[(1-benzyl-2-oxo-4-pyridyl)amino]pyrimidine-2-carbaldehyde. To a solution of 1-benzyl-4-[(2-vinylpyrimidin-4-yl)amino]pyridin-2-one (228 mg, 0.75 mmol) in THF (20 mL) and H₂O (4 mL) were added potassium tetraoxoosmium(2-) hydrate (23 mg, 0.075 mol), 4-methyl-4-oxido-morpholin-4-ium (97 mg, 0.82 mmol) and periodic acid sodium hydride (647 mg, 3.00 mmol). The mixture was stirred at 10° C. for 16 h and stirred at 30° C. for 2 h. Water was added and the mixture was filtered and the filtrate was extracted with EtOAc. The combined organic phases were washed with brine, dried over anhydrous Na₂SO₄, filtered and concentrated to give the title compound (220 mg). LCMS (ESI): m/z, 325.1 [M+H+H₂O]⁺.

7. Synthesis of 1-benzyl-4-[[2-(hydroxymethyl)pyrimidin-4-yl]amino]pyridin-2-one. To a solution of 4-[(1-benzyl-2-oxo-4-pyridyl)amino]pyrimidine-2-carbaldehyde (220 mg, 0.72 mmol) in THF (10 mL) was added sodium triacetoxyboranuide (304 mg, 1.44 mmol) and the mixture was stirred at 10° C. for 16 h. The solvent was removed to yield a residue which was purified by prep-HPLC to give the title compound (180 mg). LCMS (ESI): m/z, 309.2 [M+H]⁺.

8. Synthesis of 2-[[4-[(1-benzyl-2-oxo-4-pyridyl)amino]pyrimidin-2-yl]methyl]-1-isopropyl-1-(2,2,2-trifluoroethyl)isourea. To a solution of 1-benzyl-4-[[2-(hydroxymethyl)pyrimidin-4-yl]amino]pyridin-2-one (180 mg, 0.58 mmol) and isopropyl(2,2,2-trifluoroethyl)cyanamide (388 mg, 2.34 mmol) in 1,4-dioxane (10 mL) was added ZnCl₂ (318 mg, 2.34 mmol). The mixture was stirred at 100° C. for 4 h under N₂. The solvent was removed to yield a residue which was purified by prep-HPLC to give the title compound (2.5 mg). ¹H NMR (400 MHz, CDCl₃) δ 9.97 (br s, 1H), 8.38 (d, J=5.6 Hz, 1H), 7.64 (d, J=7.6 Hz, 1H), 7.45-7.24 (m, 5H), 7.09 (d, J=2.0 Hz, 1H), 6.81 (d, J=5.6 Hz, 1H), 6.48 (dd, J=2.4, 7.6, Hz, 1H), 5.28-5.38 (m, 1H), 5.15 (s, 2H), 5.05 (s, 2H), 4.18-4.07 (m, 3H), 1.08 (d, J=6.8 Hz, 6H); LCMS (ESI): m/z, 475.3 [M+H]⁺.

### Example 195. Preparation of Compound E131

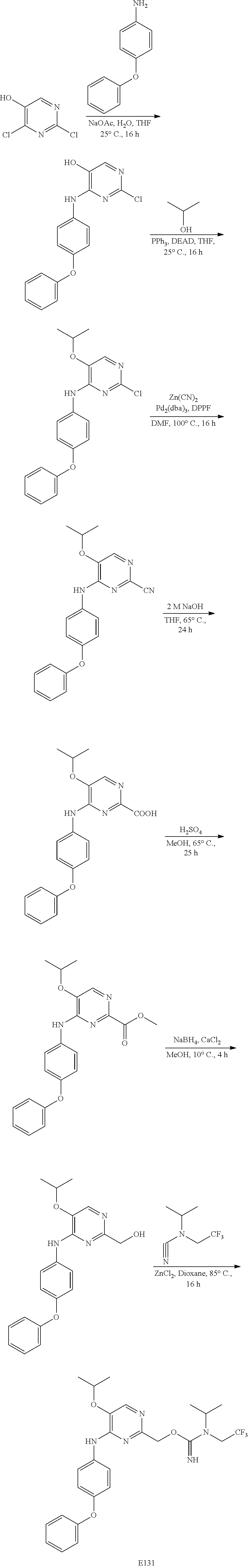

1. Synthesis of 2-chloro-4-((4-phenoxyphenyl) amino) pyrimidin-5-ol. To a solution of 2,4-dichloropyrimidin-5-ol (2.02 g, 12.27 mmol) and 4-phenoxyaniline (2.5 g, 13.50 mmol) in THF (10 mL) and H₂O (10 mL) was added NaOAc (2.01 g, 24.54 mmol) and stirred at 65° C. for 16 h. The reaction was added water and extracted with EtOAc. The organic layers were washed with brine, dried over Na₂SO₄, filtered and concentrated to give a residue which was purified by column chromatography to give title compound (2.33 g). LCMS (ESI): m/z, 314.1 [M+H]⁺.

2. Synthesis of 2-chloro-5-isopropoxy-N-(4-phenoxyphenyl) pyrimidin-4-amine. To a solution of 2-chloro-4-(4-phenoxyanilino) pyrimidin-5-ol (700 mg, 2.23 mmol) and propan-2-ol (160 mg, 2.68 mmol) was added triphenylphosphane (760 mg, 2.90 mmol) and ethyl N-ethoxycarbonyliminocarbamate (466 mg, 2.68 mmol) in THF (10 mL) at 25° C. The reaction was stirred at 25° C. for 16 h. The reaction was directly distilled under reduced pressure to obtain the crude product which was purified by column chromatography to give title compound (720 mg). LCMS (ESI): m/z, 356.1 [M+H]⁺.

3. Synthesis of 5-isopropoxy-4-((4-phenoxyphenyl) amino) pyrimidine-2-carbonitrile. To a solution of 2-chloro-5-isopropoxy-N-(4-phenoxyphenyl) pyrimidin-4-amine (900 mg, 2.53 mmol), dicyanozinc (1.19 g, 10.12 mmol) and cyclopentyl(diphenyl)phosphane; iron (140.23 mg, 0.25 mmol) was added (1E,4E)-1,5-diphenylpenta-1,4-dien-3-one; palladium (231 mg, 0.25 mol) in DMF (15 mL) at 10° C. The reaction was stirred at 100° C. for 16 h. The reaction was diluted with water, extracted with EtOAc, washed with brine, dried with Na₂SO₄, filtered and concentrated. The mixture was purified by column chromatography to give title compound (820 mg). LCMS (ESI): m/z, 347.2 [M+H]⁺.

4. Synthesis of 5-isopropoxy-4-((4-phenoxyphenyl) amino)pyrimidine-2-carboxylic acid. To a solution of 5-isopropoxy-4-(4-phenoxyanilino) pyrimidine-2-carbonitrile (700 mg, 2.02 mmol) was added in NaOH (2 M, 20 mL) in tetrahydrofuran (5 mL) at 10° C. The reaction was stirred at 65° C. for 24 h. The reaction was diluted with 2M HCl, extracted with EtOAc, washed with brine (10 mL), dried with Na₂SO₄, filtered and concentrated to give title compound (700 mg). LCMS (ESI): m/z, 366.2 [M+H]⁺.

5. Synthesis of methyl 5-isopropoxy-4-((4-phenoxyphenyl) amino) pyrimidine-2-carboxylate. To a solution of 5-isopropoxy-4-(4-phenoxyanilino) pyrimidine-2-carboxylic acid (750 mg, 2.05 mmol) was added sulfuric acid (201 mg, 2.05 mmol, 0.5 mL) in methanol (15 mL) at 25° C. The reaction was stirred at 65° C. for 24 h. The mixture was diluted with 2M aq. NaOH, extracted with EtOAc, washed with brine, dried with Na₂SO₄, filtered and concentrated. The mixture was purified with column chromatography to give title compound (650 mg). LCMS (ESI): m/z, 380.2 [M+H]⁺.

6. Synthesis of (5-isopropoxy-4-((4-phenoxyphenyl) amino) pyrimidin-2-yl) methanol. To a solution of methyl 5-isopropoxy-4-(4-phenoxyanilino) pyrimidine-2-carboxylate (300 mg, 0.79 mmol) was added sodium boranuide (119 mg, 3.16 mmol) and dichlorocalcium (53 mg, 0.47 mmol) in methanol (4 mL) under ice cold bath. The reaction was stirred at 10° C. for 4 h. The reaction was diluted with water, extracted with EtOAc, washed with brine, dried with Na₂SO₄, filtered and concentrated. The mixture was purified by column chromatography to give title compound (70 mg). LCMS (ESI): m/z, 352.2 [M+H]⁺.

7. Synthesis of compound E131. To a solution of [5-isopropoxy-4-(4-phenoxyanilino)pyrimidin-2-yl]methanol (50 mg, 0.14 mmol) and isopropyl(2,2,2-trifluoroethyl)cyanamide (70 mg, 0.43 mmol) was added dichlorozinc (77 mg, 0.57 mmol) in 1,4-dioxane (2 mL) in 25° C. The reaction was stirred at 80° C. for 24 h. The reaction was diluted with water, extracted with EtOAc, washed with brine, dried with Na₂SO₄, filtered and concentrated. The mixture was purified by prep-HPLC to give title compound (5 mg). ¹H NMR (400 MHz, CDCl₃) δ 7.87 (s, 1H), 7.71-7.64 (m, 2H), 7.36-7.29 (m, 2H), 7.19 (s, 1H), 7.09 (t, J=7.6 Hz, 1H), 7.05-6.96 (m, 4H), 5.13 (s, 2H), 4.66 (h, J=6.0 Hz, 1H), 4.25 (m, J=6.8 Hz, 1H), 3.90 (q, J=8.8 Hz, 2H), 1.44 (d, J=6 Hz, 6H), 1.19 (d, J=6.8 Hz, 6H). LCMS (ESI): m/z, 518.3 [M+H]⁺.

### Example 196. Preparation of Compound E132

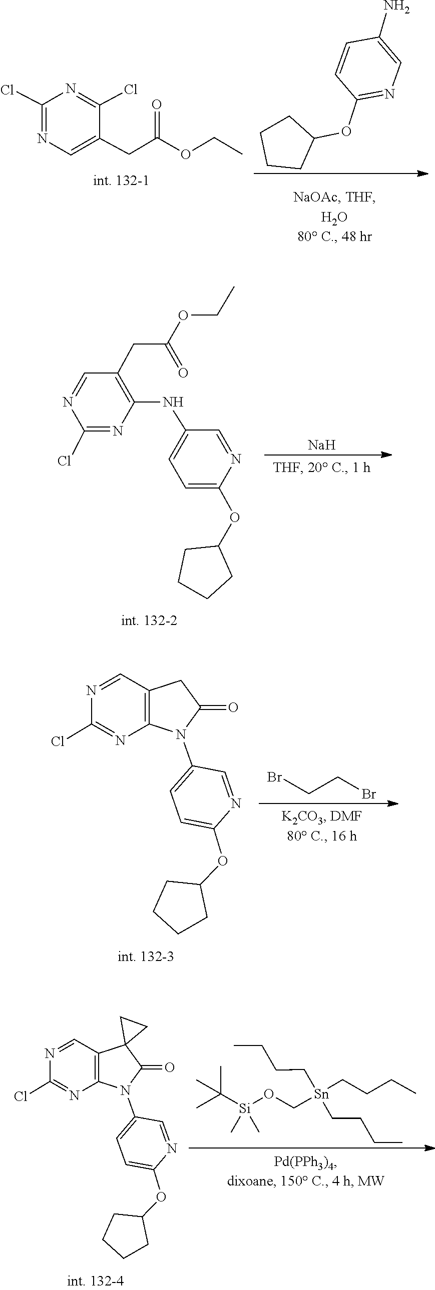

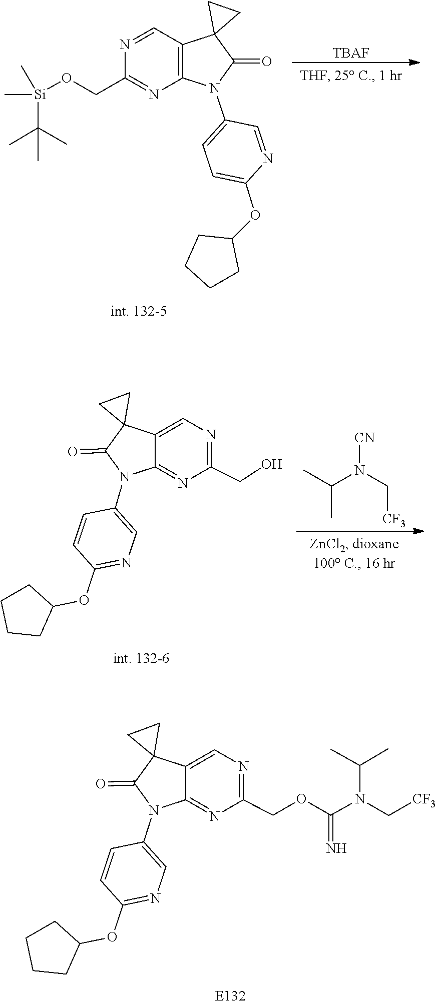

1. Synthesis of compound int. 132-2. To a solution of ethyl 2-(2,4-dichloropyrimidin-5-yl)acetate (1.5 g, 6.38 mmol) and 6-(cyclopentyloxy)pyridin-3-amine (1.14 g, 6.38 mmol) in THF (3 mL) and H₂O (3 mL) was added NaOAc (1.05 g, 12.76 mmol) and stirred at 80° C. for 48 h. The reaction was added water and extracted with EtOAc. The organic layers were washed with brine (50 mL), dried over Na₂SO₄, filtered and concentrated to give a residue. The residue was purified by column chromatography to get title compound (1.53 g).

2. Synthesis of compound int. 132-3. To a solution of ethyl 2-(2-chloro-4-((6-(cyclopentyloxy)pyridin-3-yl)amino)pyrimidin-5-yl)acetate (1.53 g, 4.06 mmol) in THF (20 mL) was added NaH (325 mg, 8.12 mmol, 60% purity) and stirred at 20° C. for 1 hour. After the reaction mixture was cooled to 0° C., the reaction mixture was quenched by addition of H₂O. The reaction was extracted with EtOAc. The organic layers were washed with brine, dried over Na₂SO₄, filtered and concentrated to give a residue. The residue was purified by column chromatography to afford title compound (1.02 g).

3. Synthesis of compound int. 132-4. To a solution of 2-chloro-7-(6-(cyclopentyloxy)pyridin-3-yl)-5,7-dihydro-6H-pyrrolo[2,3-d]pyrimidin-6-one (1.02 g, 3.08 mmol) in DMA (10 mL) was added K₂CO₃ (852 mg, 6.17 mmol) and then 1,2-dibromoethane (1.16 g, 6.17 mmol) was added and stirred at 80° C. for 16 hours. The reaction was filtered and concentrated to give a residue. The residue was purified by column chromatography to afford title compound (676 mg).

4. Synthesis of compound int. 132-5. To a solution of 2′-chloro-7′-(6-(cyclopentyloxy)pyridin-3-yl)spiro[cyclopropane-1,5′-pyrrolo[2,3-d]pyrimidin]-6′(7′H)-one (0.3 g, 0.84 mmol) and tert-butyldimethyl((tributylstannyl)methoxy)silane (403 mg, 0.92 mmol) in dioxane (1 mL) was added Pd(PPh₃)₄ (291 mg, 0.25 mmol) and stirred at 150° C. for 4 hours by microwave. The reaction was filtered and concentrated to give a residue. The residue was purified by column chromatography to afford title compound (92 mg).

5. Synthesis of compound int. 132-6. To a mixture of 2′-(((tert-butyldimethylsilyl)oxy)methyl)-7′-(6-(cyclopentyloxy)pyridin-3-yl)spiro[cyclopropane-1,5′-pyrrolo[2,3-d]pyrimidin]-6′(7′H)-one (82 mg, 0.18 mmol) in THF (1 mL) was added TBAF (1 M, 0.53 mL) at 25° C. The mixture was stirred at 25° C. for 1 hour. The reaction was filtered and concentrated to give a residue. The residue was purified by column chromatography to afford title compound (27 mg).

6. Synthesis of compound E132. To a solution of 7′-(6-(cyclopentyloxy)pyridin-3-yl)-2′-(hydroxymethyl)spiro[cyclopropane-1,5′-pyrrolo[2,3-d]pyrimidin]-6′(7′H)-one (27 mg, 0.08 mmol) and N-isopropyl-N-(2,2,2-trifluoroethyl)cyanamide (38 mg, 0.23 mmol) in Dioxane (1 mL) was added ZnCl₂ (42 mg, 0.31 mmol) and stirred at 100° C. for 16 hours under N₂. The reaction was filtered and concentrated to give a residue. The residue was purified by HPLC to afford title compound (8 mg). ¹H NMR (400 MHz, CDCl₃) δ 8.34 (d, J=2.0 Hz, 1H), 8.08 (s, 1H), 7.70 (dd, J=8.4, 2.4 Hz, ¹H), 6.80 (br d, J=9.2 Hz, ¹H), 5.50-5.38 (m, 1H), 5.26 (s, 2H), 4.09 (td, J=6.8, 13.2 Hz, 1H), 3.89-3.80 (m, 2H), 1.98 (br d, J=3.6 Hz, 4H), 1.86-1.77 (m, 8H), 1.18 (br d, J=6.8 Hz, 6H). LCMS (ESI): m/z 519.3 [M+H]⁺

### Example 197. Preparation of Compound E133

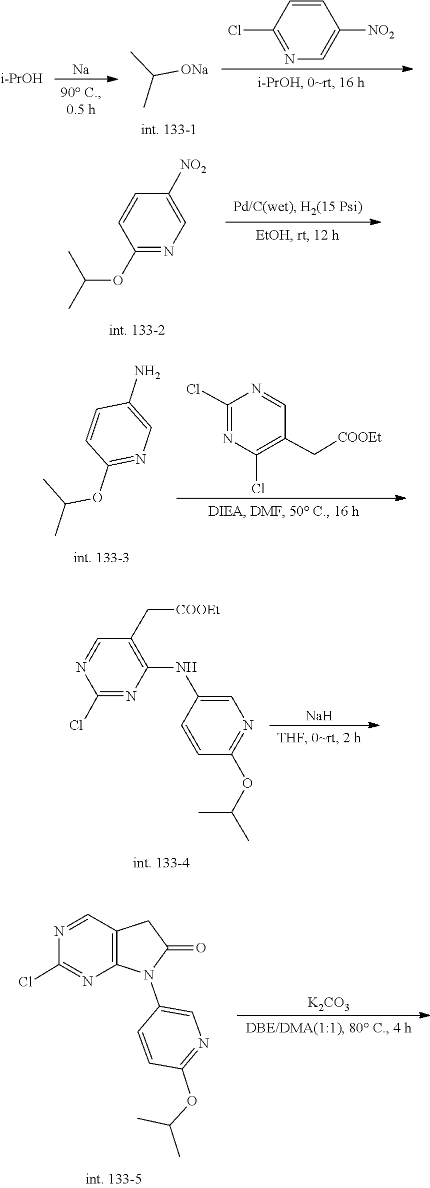

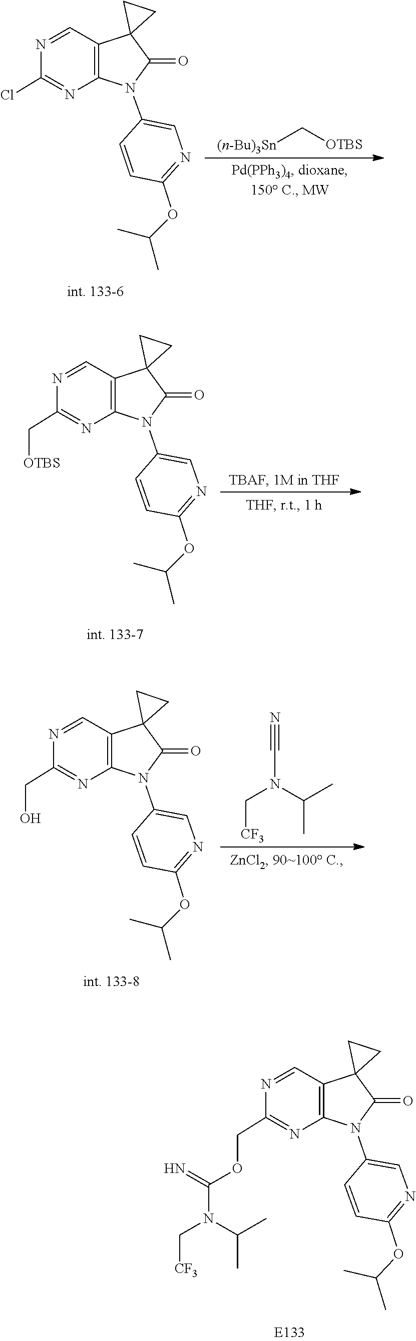

1. Synthesis of compound int. 133-1. To i-PrOH (5 mL) was added sodium (150 mg, 6.52 mmol), the mixture was stirred at 90° C. for 0.5 hours to give title compound (536 mg crude) in i-PrOH (5 mL).

2. Synthesis of compound int. 133-2. To a solution of 2-chloro-5-nitro-pyridine (500 mg, 3.15 mmol) in iPrOH (5 mL) was added i-PrONa (530 mg, 6.46 mmol) (in 10 mL i-PrOH and 5 mL THF) drop slowly at 0° C., the mixture was warmed to 15° C. for 16 hours. The residue was concentrated. The residue was purified by column chromatography to afford title compound (439 mg). ¹H NMR (400 MHz, DMSO-d₆) δ ppm 9.07 (d, J=2.8 Hz, 1H) 8.44 (dd, J=9.2, 2.8 Hz, 1H) 6.95 (d, J=9.2 Hz, 1H) 5.42-5.31 (m, 1H) 1.33 (d, J=6.4 Hz, 6H).

3. Synthesis of compound int. 133-3. To a solution of 2-isopropoxy-5-nitro-pyridine (2.57 g, 14.11 mmol) in EtOH (50 mL) was added Pd/C (10%) (800 mg, 7.52 mmol), the mixture was stirred at 15° C. for 12 hours with H₂ (15 Psi). The residue was filtered over of celite and concentrated to give title compound (2.03 g). ¹H NMR (400 MHz, DMSO-d₆) δ 7.48 (d, J=2.80 Hz, 1H) 6.94-6.99 (m, 1H) 6.45 (d, J=8.80 Hz, 1H) 4.97-5.05 (m, 1H) 4.69 (s, 2H) 1.20 (d, J=6.40 Hz, 6H).

4. Synthesis of compound int. 133-4. To a mixture of ethyl 2-(2,4-dichloropyrimidin-5-yl)acetate (1.54 g, 6.57 mmol) and DIEA (2.17 mL) in DMF (20 mL) was added 6-isopropoxypyridin-3-amine (1 g, 6.57 mmol). The mixture was heated at 50° C. for 12 hours. The residue was concentrated. The residue was concentrated to give title compound (1.4 g).

5. Synthesis of compound int. 133-5. A solution of ethyl 2-(2-chloro-4-((6-isopropoxypyridin-3-yl)amino)pyrimidin-5-yl)acetate (380 mg) in THF (8 mL) was added NaH (60% in mineral oil) (87 mg, 2.17 mmol, 60% purity), the mixture was stirred at 20° C. for 2 hours. To the mixture was added brine and EtOAc, the aqueous phase was extracted with EtOAc. The combined organic layers were dried over Na₂SO₄, filtered and concentrated. The residue was concentrated to give title compound (220 mg).

6. Synthesis of compound int. 133-6. To the mixture of 2-chloro-7-(6-isopropoxypyridin-3-yl)-5,7-dihydro-6H-pyrrolo[2,3-d]pyrimidin-6-one (340 mg, 1.12 mmol) and 1,2-dibromoethane (6.54 g, 34.81 mmol, 3 mL) in DMA (3 mL) was added K₂CO₃ (308 mg, 2.23 mmol), the mixture was stirred at 80° C. for 4 hours under N₂. To the mixture was added EtOAc, the mixture was washed with brine and dried over Na₂SO₄, filtered and concentrated. The residue was purified by Prep-HPLC to give title compound (400 mg).

7. Synthesis of compound int. 133-7. To a mixture of 2′-chloro-7′-(6-isopropoxy-3-pyridyl)spiro[cyclopropane-1,5′-pyrrolo[2,3-d]pyrimidine]-6′-one (100 mg, 0.30 mmol) and tert-butyldimethyl((tributylstannyl)methoxy)silane (158 mg, 0.36 mmol) in dioxane (2 mL) was added Pd(PPh₃)₄ (70 mg, 0.06 mmol). The mixture was heated at 150° C. for 4 h under N₂ under M.W. The residue was filtered and concentrated. The crude product was purified by column chromatography to give title compound (100 mg).

8. Synthesis of compound int. 133-8. To a mixture of 2′-(((tert-butyldimethylsilyl)oxy)methyl)-7′-(6-isopropoxypyridin-3-yl)spiro[cyclopropane-1,5′-pyrrolo[2,3-d]pyrimidin]-6′(7′H)-one (140 mg, 0.32 mmol) in THF (5 mL) was added TBAF (1 M, 0.95 mL), the mixture was stirred at 25° C. for 1 hour. To the mixture was added H₂O and EtOAc, the aqueous phase was extracted with EtOAc. The combined organic layers were washed with brine and dried over Na₂SO₄, filtered and concentrated. The crude product was purified by column chromatography to give title compound (12 mg).

9. Synthesis of compound E133. To a solution of 2′-(hydroxymethyl)-7′-(6-isopropoxypyridin-3-yl)spiro[cyclopropane-1,5′-pyrrolo[2,3-d]pyrimidin]-6′(7′H)-one (11 mg, 0.03 mmol) in dioxane (1 mL) was added N-isopropyl-N-(2,2,2-trifluoroethyl)cyanamide (22 mg, 0.13 mmol) and ZnCl₂ (18 mg, 0.13 mmol), the mixture was heated at 100° C. for 6 hours with N₂. The mixture was heated at 90° C. for 2 hours with N₂. The residue was filtered and concentrated. The residue was purified by Prep-HPLC to give title compound (5.2 mg). LCMS (ESI): m/z, 493.3 [M+H]⁺. ¹H NMR (400 MHz, CDCl₃) δ 8.32 (d, J=2.4 Hz, 1H), 8.08 (s, 1H), 7.70 (dd, J=8.8, 2.4 Hz, 1H), 6.80 (d, J=8.8 Hz, 1H), 5.39-5.31 (m, 1H), 5.26 (s, 2H), 4.13-4.03 (m, I H), 3.88-3.79 (m, 2H), 2.00-1.97 (m, 2H), 1.80-1.78 (m, 2H), 1.38 (d, J=6.0 Hz, 6H), 1.17 (d, J=6.8 Hz, 6H).

### Example 198. Preparation of Compound E134

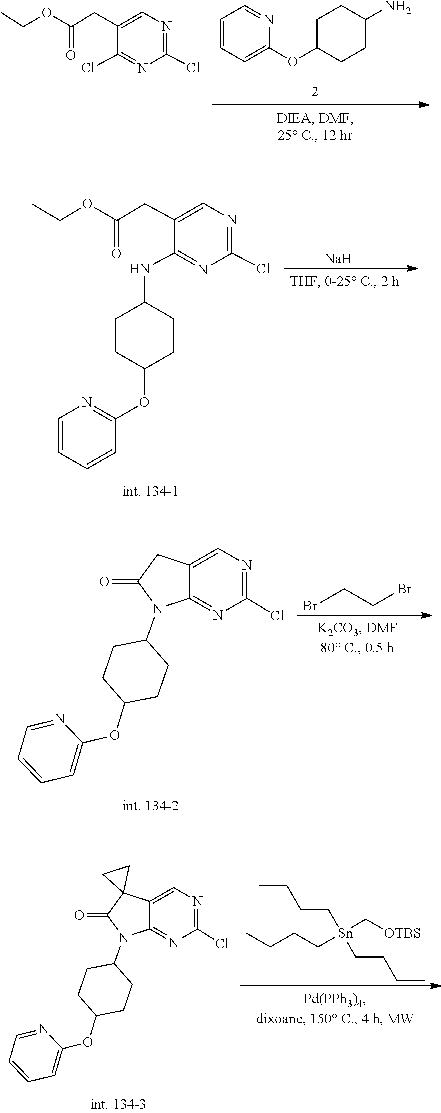

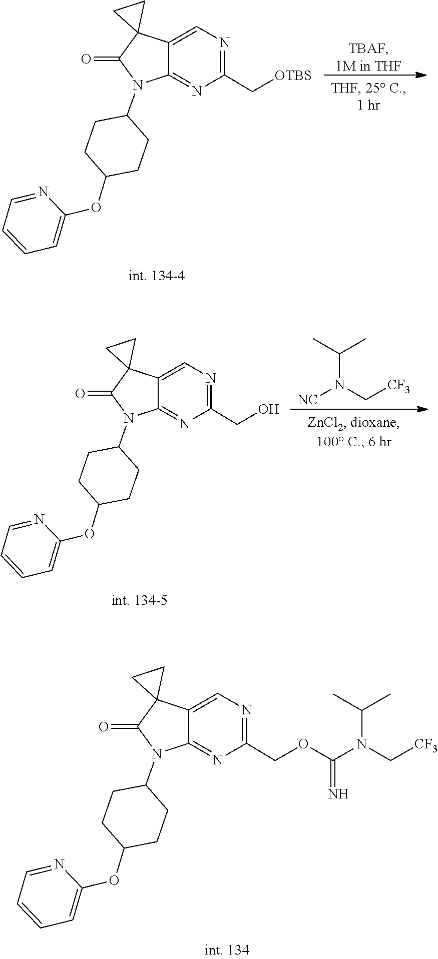 [SMILES: CC(C)(C)[Si](C)(C)OCc1ncc2c(n1)N(C1CCC(Oc3ccccn3)CC1)C(=O)C21CC1 ; "TBAF,1M in THF" ; O=C1N(C2CCC(Oc3ccccn3)CC2)c2nc(CO)ncc2C12CC2 ; CC(C)N(C#N)CC(F)(F)F ; CC(C)N(CC(F)(F)F)C(=N)OCc1ncc2c(n1)N(C1CCC(Oc3ccccn3)CC1)C(=O)C21CC1]

Synthesis of compound int. 134-1. A mixture of 4-(2-pyridyloxy)cyclohexanamine (1.17 g, 6.06 mmol), ethyl 2-(2,4-dichloropyrimidin-5-yl)acetate (1.5 g, 6.06 mmol) and DIEA (1.57 g, 12.12 mmol) in DMF (15 mL) was stirred at 25° C. for 12 h. The reaction mixture was extracted with EtOAc. The organic layers were washed with brine, dried over Na₂SO₄, filtered and concentrated under reduced pressure to give the residue. The crude product was purified by column chromatography to give title compound (2.0 g). ¹H NMR (400 MHz, CDCl₃) δ 8.15 (d, J=4.0 Hz, 1H), 7.83 (s, 1H), 7.70-7.66 (m, 1H), 7.16 (d, J=8.0 Hz, 1H), 6.92-6.96 (m, 1H), 6.75 (d, J=8.0 Hz, 1H), 5.19 (br s, 1H), 4.12-4.00 (m, 3H), 3.63 (s, 2H), 1.99-1.95 (m, 2H), 1.86-1.77 (m, 2H), 1.77-1.65 (m, 6H), 1.19 (t, J=7.2 Hz, 3H).

2. Synthesis of compound int. 134-2. To a solution of ethyl 2-(2-chloro-4-((4-(pyridin-2-yloxy)cyclohexyl)amino)pyrimidin-5-yl)acetate (1.2 g, 3.07 mmol) in THF (10 mL) was added NaH (147 mg, 6.14 mmol) at 0° C. The reaction mixture was stirred at 25° C. for 2 h under N₂. The reaction mixture was quenched by addition of H₂O (1 mL) at 0° C. and concentrated under reduced pressure. The crude product was purified by column chromatography to give title compound (830 mg).

3. Synthesis of compound int. 134-3. To a solution of 2-chloro-7-(4-(pyridin-2-yloxy)cyclohexyl)-5,7-dihydro-6H-pyrrolo[2,3-d]pyrimidin-6-one (530 mg, 1.54 mmol) in DMF (10 mL) was added 1,2-dibromoethane (866 mg, 4.61 mmol) and K₂CO₃ (1.06 g, 7.69 mmol). Then the reaction was stirred at 80° C. for 0.5 h under N₂. The reaction mixture was extracted with EtOAc. The organic layers were washed with brine, dried over Na₂SO₄, filtered and concentrated under reduced pressure to give the residue. The crude product was purified by column chromatography to give title compound (430 mg).

4. Synthesis of compound int. 134-4. To a solution of 2′-chloro-7′-(4-(pyridin-2-yloxy)cyclohexyl)spiro[cyclopropane-1,5′-pyrrolo[2,3-d]pyrimidin]-6′(7′H)-one (100 mg, 0.27 mmol) in Dioxane (3 mL) was added tert-butyl-dimethyl-(tributylstannylmethoxy)silane (176 mg, 0.40 mmol), Pd(PPh₃)₄ (31. mg, 0.027 mmol) under N₂. The reaction mixture was stirred with microwave at 150° C. for 4 h under argon. The reaction was concentrated to give a yellow solid. The crude product was purified by column chromatography to give title compound (65 mg).

5. Synthesis of compound int. 134-5. To a solution of 2′-(((tert-butyldimethylsilyl)oxy)methyl)-7′-(4-(pyridin-2-yloxy)cyclohexyl)spiro[cyclopropane-1,5′-pyrrolo[2,3-d]pyrimidin]-6′(7′H)-one (60 mg, 0.12 mmol) in THF (2 mL) was added TBAF (1 M in THF) (1 M, 0.25 mL) at 25° C. under N₂. The reaction was stirred at 25° C. for 2 h. The reaction was added water and extracted with EtOAc. The organic layers were concentrated. The crude product was purified by column chromatography to give title compound (30 mg).

6. Synthesis of compound E134. A mixture of 2′-(hydroxymethyl)-7′-(4-(pyridin-2-yloxy)cyclohexyl)spiro[cyclopropane-1,5′-pyrrolo[2,3-d]pyrimidin]-6′(7′H)-one (30 mg, 0.082 mmol), isopropyl(2,2,2-trifluoroethyl)cyanamide (54 mg, 0.33 mmol) and ZnCl₂ (33 mg, 0.25 mmol) in Dioxane (3 mL) was stirred at 100° C. for 6 h under N₂. The reaction was concentrated. The residue was purified by prep-HPLC to give title compound (5 mg). LCMS (ESI): m/z 533.3 [M+H]⁺.

### Example 199. Preparation of Compound E135

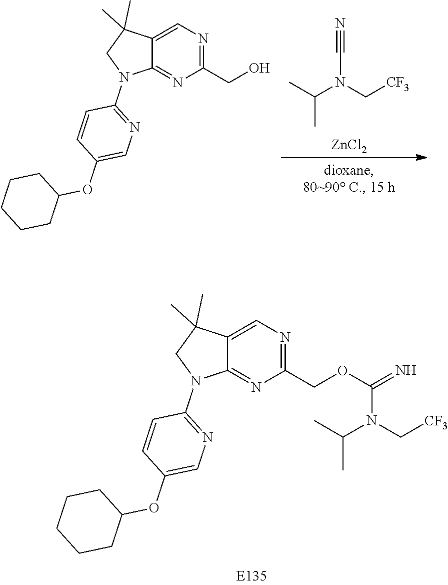

To a solution of (7-(5-(cyclohexyloxy)pyridin-2-yl)-5,5-dimethyl-6,7-dihydro-5H-pyrrolo[2,3-d]pyrimidin-2-yl)methanol (10 mg, 0.03 mmol) in dioxane (1 mL) was added N-isopropyl-N-(2,2,2-trifluoroethyl)cyanamide (19 mg, 0.11 mmol) and ZnCl₂ (15 mg, 0.11 mmol), the mixture was heated at 80° C. for 12 hours under N₂. To the mixture was added N-isopropyl-N-(2,2,2-trifluoroethyl)cyanamide (28 mg, 0.17 mmol) and ZnCl₂ (23 mg, 0.17 mmol), the mixture was heated at 90° C. for 3 hours under N₂. The residue was purified by Prep-HPLC to give title compound (5 mg). LCMS (ESI): m/z, 521.3 [M+H]⁺.

### Example 200. Preparation of Compound E136

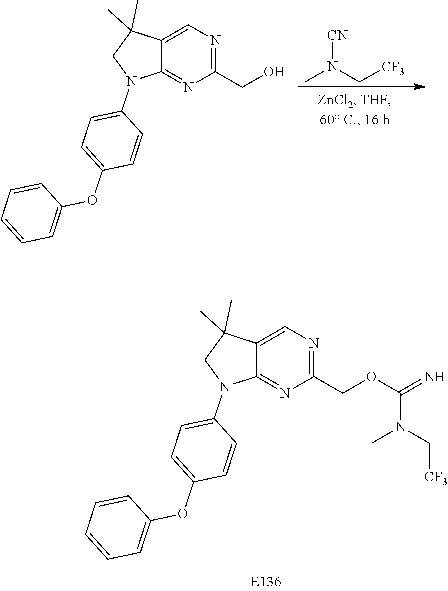

To a solution of [5,5-dimethyl-7-(4-phenoxyphenyl)-6H-pyrrolo[2,3-d]pyrimidin-2-yl]methanol (50 mg, 0.14 mmol) and methyl(2,2,2-trifluoroethyl)cyanamide (60 mg, 0.43 mmol) in THF (2 mL) was added ZnCl₂ (78 mg, 0.58 mmol). The mixture was stirred at 60° C. for 16 h under N₂. The mixture was filtered and the filtrate was purified by prep-HPLC to give title compound (50 mg). ¹H NMR (400 MHz, CDCl₃) δ 8.05 (s, 1H), 7.77 (d, J=8.8 Hz, 2H), 7.40-7.28 (m, 2H), 7.18-6.98 (m, 5H), 5.19 (s, 2H), 3.97 (q, J=8.8 Hz, 2H), 3.80 (s, 2H), 3.03 (s, 3H), 1.43 (s, 6H). LCMS (ESI): m/z, 486.2 [M+H]⁺.

### Example 201. Preparation of Compound E137

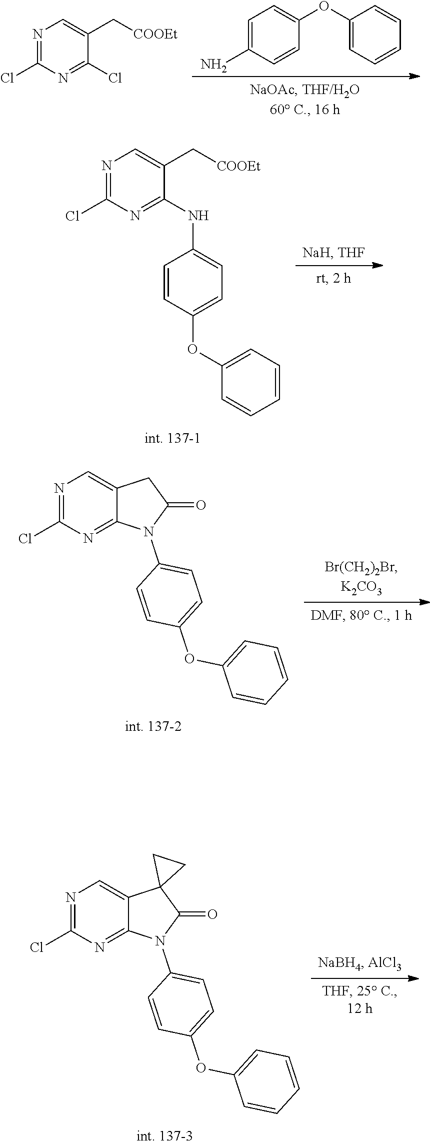 [SMILES: CCOC(=O)Cc1cnc(Cl)nc1Cl ; Nc1ccc(Oc2ccccc2)cc1 ; CCOC(=O)Cc1cnc(Cl)nc1Nc1ccc(Oc2ccccc2)cc1 ; O=C1Cc2cnc(Cl)nc2N1c1ccc(Oc2ccccc2)cc1 ; O=C1N(c2ccc(Oc3ccccc3)cc2)c2nc(Cl)ncc2C12CC2]

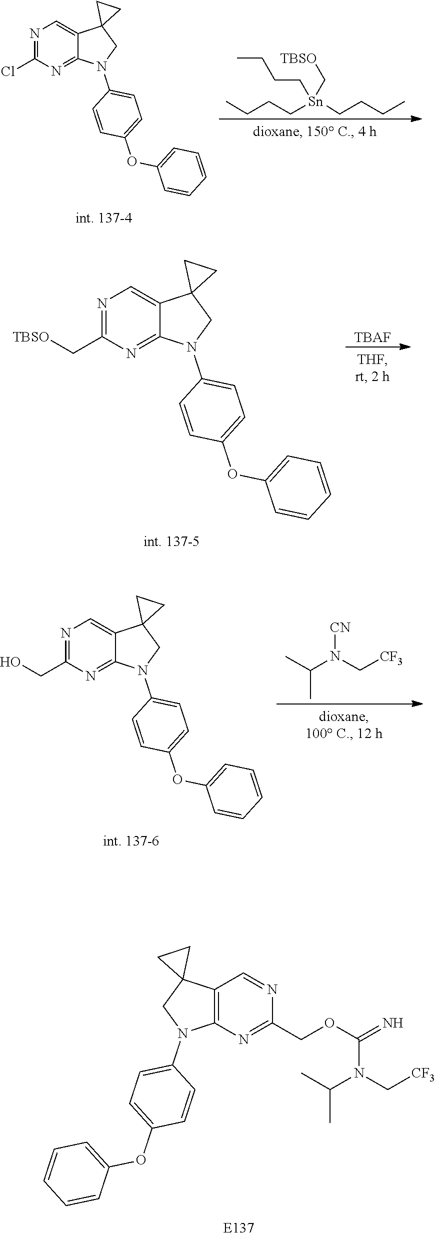

Synthesis of compound int. 137-1. To a solution of ethyl 2-(2,4-dichloropyrimidin-5-yl)acetate (1.5 g, 6.38 mmol) in THF (20 mL) and water (10 mL) was added 4-phenoxyaniline (1.42 g, 7.66 mmol) and NaOAc (1.31 g, 15.95 mmol) at 25° C. The mixture was stirred at 60° C. for 12 h. Water was added and extracted with EtOAc. The combined organic phases were washed with brine, dried over anhydrous Na₂SO₄, filtered and concentrated. The residue was purified by column chromatography to give title compound (1.0 g). ¹H NMR (400 MHz, CDCl3) δ 8.35 (s, 1H), 8.02 (s, 1H), 7.60-7.54 (m, 2H), 7.34 (m, 2H), 7.13-7.08 (m, 1H), 7.06-6.98 (m, 4H), 4.23 (q, J=7.2 Hz, 2H), 3.53 (s, 2H), 1.31 (t, J=7.2 Hz, 3H). LCMS (ESI): m/z, 384.1 [M+H]⁺.

2. Synthesis of compound int. 137-2. To a solution of ethyl 2-[2-chloro-4-(4-phenoxyanilino)pyrimidin-5-yl]acetate (1.0 g, 2.61 mmol) in THF (20 mL) was added NaH (157 mg, 3.91 mmol, 60% purity) at 0° C. The reaction was stirred at 25° C. for 2 h. The reaction mixture was diluted with saturated NH₄Cl (30 mL), H₂O (30 mL) and extracted with EtOAc. The combined organic phases were washed with brine, dried over anhydrous Na₂SO₄, filtered and concentrated. The residue was purified by column chromatography to give title compound (0.75 g). LCMS (ESI): m/z, 338.1, 340.0 [M+H]⁺.

3. Synthesis of compound int. 137-3. To a solution of 2-chloro-7-(4-phenoxyphenyl)-5H-pyrrolo[2,3-d]pyrimidin-6-one (0.75 g, 2.22 mmol) in DMF (12 mL) was added 1,2-dibromoethane (1.25 g, 6.66 mmol) and K₂CO₃ (1.53 g, 11.10 mmol). The reaction was stirred at 80° C. for 0.5 h. The reaction was diluted with NH₄Cl, brine and extracted with EA, washed with brine, dried with Na₂SO₄, filtered and concentrated. The residue was purified by column chromatography to give title compound (0.6 g). ¹H NMR (400 MHz, CDCl₃) δ: 7.94 (s, 1H), 7.50-7.44 (m, 2H), 7.42-7.36 (m, 2H), 7.21-7.15 (m, 1H), 7.15-7.09 (m, 4H), 2.02 (q, J=4.4 Hz, 2H), 1.81 (q, J=4.4 Hz, 2H). LCMS (ESI): m/z, 364.1 [M+H]⁺.

4. Synthesis of compound int. 137-4. To a mixture of 2′-chloro-7′-(4-phenoxyphenyl)spiro[cyclopropane-1,5′-pyrrolo[2,3-d]pyrimidine]-6′-one (0.6 g, 1.65 mmol) in THF (20 mL) was added NaBH₄ (250 mg, 6.60 mmol), followed by AlCl₃ (880 mg, 6.60 mmol). The mixture was stirred at 25° C. for 12 h. The reaction was quenched by H₂O and extracted with EA, washed with brine, dried with Na₂SO₄, filtered and concentrated. The residue was purified by column chromatography to give title compound (0.24 g). ¹H NMR (400 MHz, CDCl₃) δ 7.74-7.69 (m, 2H), 7.46 (s, 1H), 7.38-7.32 (m, 2H), 7.14-7.06 (m, 3H), 7.05-7.00 (m, 2H), 4.13 (s, 2H), 1.21 (m, 4H). LCMS (ESI): m/z, 350.1, 352.1 [M+H]⁺.

5. Synthesis of compound int. 137-5. To a mixture of 2-chloro-7-(4-phenoxyphenyl)spiro[6H-pyrrolo[2,3-d]pyrimidine-5,1′-cyclopropane](0.22 g, 0.63 mol) in dioxane (4 mL) was added tert-butyl-dimethyl-(tributylstannylmethoxy)silane (356 mg, 0.82 mol) and Pd(PPh₃)₄ (73 mg, 0.063 mol). The mixture was purged by N₂ and stirred at 150° C. for 4 h under microwave. The reaction mixture was concentrated under reduced pressure. The residue was purified by column chromatography to give title compound (90 mg). ¹H NMR (400 MHz, CDCl₃) δ 7.89 (d, J=9.2 Hz, 2H), 7.64 (s, 1H), 7.36-7.31 (m, 2H), 7.11-7.04 (m, 3H), 7.00 (d, J=8.0 Hz, 2H), 4.77 (s, 2H), 4.07 (s, 2H), 1.24-1.19 (m, 2H), 1.17-1.12 (m, 2H), 0.97 (s, 9H), 0.15 (s, 6H). LCMS (ESI): m/z, 460.3 [M+H]⁺.

6. Synthesis of compound int. 137-6. To a mixture of tert-butyl-dimethyl-[[7-(4-phenoxyphenyl)spiro[6H-pyrrolo[2,3-d]pyrimidine-5,1′-cyclopropane]-2-yl]methoxy]silane (90 mg, 0.19 mol) in THF (2 mL) was added TBAF (1 M, 0.59 mL) at 25° C. The mixture was stirred at 25° C. for 1. The reaction mixture was concentrated and purified by prep-TLC to give title compound (40 mg). 1H NMR (400 MHz, CDCl3) δ: 7.77-7.73 (m, 2H), 7.62 (s, 1H), 7.38-7.31 (m, 2H), 7.14-7.05 (m, 3H), 7.02 (d, J=8.0 Hz, 2H), 4.65 (s, 2H), 4.11 (s, 2H), 1.24-1.21 (m, 2H), 1.20-1.15 (m, 2H). LCMS (ESI): m/z, 346.1 [M+H]⁺.

7. Synthesis of compound E137. To a mixture of tert-butyl-dimethyl-[[7-(4-phenoxyphenyl)spiro[6H-pyrrolo[2,3-d]pyrimidine-5,1′-cyclopropane]-2-y1]methoxy]silane (90 mg, 0.19 mol) in THF (2 mL) was added TBAF (1 M, 0.59 mL) at 25° C. The mixture was stirred at 25° C. for 1 h. The reaction mixture was concentrated and purified by prep-TLC to give title compound (40 mg). ¹H NMR (400 MHz, CDCl₃) δ 7.77-7.73 (m, 2H), 7.62 (s, 1H), 7.38-7.31 (m, 2H), 7.14-7.05 (m, 3H), 7.02 (d, J=8.0 Hz, 2H), 4.65 (s, 2H), 4.11 (s, 2H), 1.24-1.21 (m, 2H), 1.20-1.15 (m, 2H). LCMS (ESI): m/z, 346.1 [M+H]⁺

### Example 202. Preparation of Compound E138

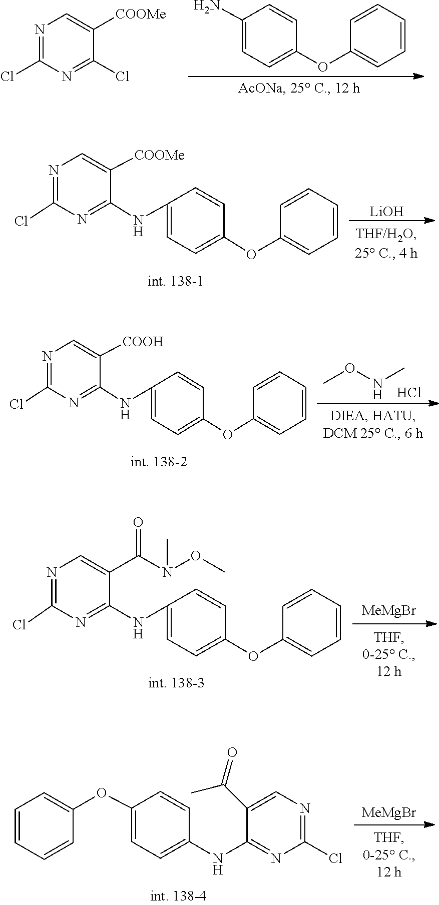

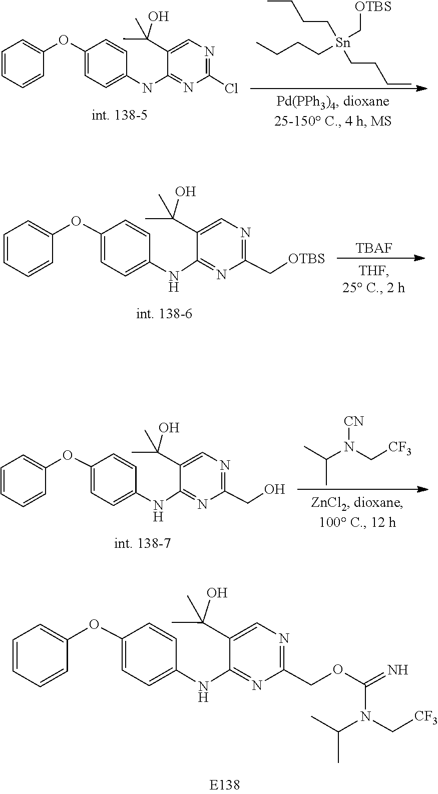

Synthesis of compound int. 138-1. To a solution of methyl 2,4-dichloropyrimidine-5-carboxylate (1 g, 4.83 mmol) in THF (20 mL) and water (10 mL) was added 4-phenoxyaniline (904 mg, 4.88 mmol) and AcONa (991 mg, 12.08 mmol) at 25° C. The mixture was stirred at 25° C. for 12 h. The mixture was filtered and washed with H₂O, EA. The filter cake was concentrated in vacuum to give title compound (1.25 g). LCMS (ESI): m/z, 356.1 [M+H]⁺.

2. Synthesis of compound int. 138-2. To a solution of methyl 2-chloro-4-(4-phenoxyanilino)pyrimidine-5-carboxylate (0.2 g, 0.56 mmol) in THF (2 mL) and H₂O (2 mL) at 25° C. was added LiOH (47 mg, 1.12 mmol). The reaction was stirred at 25° C. for 4 h. The reaction mixture was diluted with 2 M HCl to pH ˜3, extracted with EA, washed with brine (5 mL), dried with Na₂SO₄, filtered and concentrated to give title compound (0.19 g). LCMS (ESI): m/z, 342.1, 344.1 [M+H]⁺. 1H NMR (400 MHz, DMSO-d6) δ 10.55 (s, 1H), 7.60-7.66 (m, 2H), 8.76 (s, 1H), 7.36-7.44 (m, 2H), 7.12-7.17 (m, 1H), 7.00-7.09 (m, 4H).

3. Synthesis of compound int. 139-3. To a solution of 2-chloro-4-(4-phenoxyanilino)pyrimidine-5-carboxylic acid (0.19 g, 0.56 mmol) in DCM (5 mL) was added N-methoxymethanamine hydrochloride (81 mg, 0.83 mmol), DIEA (323 mg, 2.50 mmol) and HATU (254 mg, 0.67 mmol). The reaction was stirred at 25° C. for 6 h. The reaction mixture was concentrated and purified by column chromatography to give title compound (0.18 g). 1H NMR (400 MHz, CDCl₃) δ 10.06 (s, 1H), 8.77 (s, 1H), 7.58-7.63 (m, 2H), 7.32-7.38 (m, 2H), 7.09-7.14 (m, 1H), 7.03 (m, 4H), 3.67 (s, 3H), 3.42 (s, 3H). LCMS (ESI): m/z, 385.1, 387.1 [M+H]⁺.

4. Synthesis of compound int. 138-4. To a solution of 2-chloro-N-methoxy-N-methyl-4-(4-phenoxyanilino)pyrimidine-5-carboxamide (0.65 g, 1.69 mmol) in THF (10 mL) was added dropwise bromo(methyl)magnesium (1 M, 8.45 mL) at 0° C. The reaction mixture was stirred at 25° C. for 12 h. The reaction mixture was quenched by sat. NH₄Cl, water and extracted with EA. The combined organic phase was washed with brine, dried over anhydrous Na₂SO₄, filtered and concentrated. The residue was purified by column chromatography to give title compound (0.4 g). ¹H NMR (400 MHz, CDCl₃) δ 8.78 (s, 1H), 7.60-7.66 (m, 2H), 7.33-7.39 (m, 2H), 7.10-7.16 (m, 1H), 7.02-7.07 (m, 4H), 2.66 (s, 3H). LCMS (ESI): m/z, 340.1, 342.0 [M+H]⁺.

5. Synthesis of compound int. 138-5. To a solution of 1-[2-chloro-4-(4-phenoxyanilino)pyrimidin-5-yl]ethan-1-one (0.4 g, 1.18 mmol) in THF (8 mL) was added dropwise bromo(methyl)magnesium (1 M, 4.71 mL) at 0° C. The reaction mixture was stirred at 25° C. for 12 h. The reaction mixture was quenched by saturated NH₄Cl, water and extracted with EA. The combined organic phase was washed with saturated brine, dried over anhydrous Na₂SO₄, filtered and concentrated. The residue was purified by column chromatography to give title compound (0.4 g). ¹H NMR (400 MHz, CDCl3) δ: 9.33 (s, 1H), 7.96 (s, 1H), 7.61-7.56 (m, 2H), 7.34 (m, 2H), 7.14-7.08 (m, 1H), 7.05-7.00 (m, 4H), 1.70 (s, 6H). LCMS (ESI): m/z, 356.1, 358.1 [M+H]⁺.

6. Synthesis of compound int. 138-6. To a mixture of 2-[2-chloro-4-(4-phenoxyanilino)pyrimidin-5-yl]propan-2-ol (60 mg, 0.17 mol) in dioxane (2 mL) was added tert-butyl-dimethyl-(tributylstannylmethoxy)silane (88 mg, 0.20 mol) and Pd(PPh₃)₄ (20 mg, 0.017 mol). The mixture was purged by N₂ and stirred at 150° C. for 4 h under microwave. The reaction mixture was diluted with brine and extracted with EA. The combined organic phase was washed with brine, dried over anhydrous Na₂SO₄, filter and concentrated in vacuum to give a residue which was purified by prep-TLC to give title compound (40 mg). LCMS (ESI): m/z, 466.3 [M+H]⁺.

7. Synthesis of compound int. 138-7. To a mixture of 2-[2-[[tert-butyl(dimethyl)silyl]oxymethyl]-4-(4-phenoxyanilino)pyrimidin-5-yl]propan-2-ol (25 mg, 0.054 mol) in THF (1 mL) was added TBAF (1 M, 0.16 mL) at 25° C. The mixture was stirred at 25° C. for 1 h. The reaction mixture was concentrated to give a residue which was purified by prep-TLC to give title compound (15 mg). LCMS (ESI): m/z, 352.2 [M+H]⁺.

8. Synthesis of compound E138. To a solution of 2-[2-(hydroxymethyl)-4-(4-phenoxyanilino)pyrimidin-5-yl]propan-2-ol (15 mg, 0.043 mol) and isopropyl(2,2,2-trifluoroethyl)cyanamide (21 mg, 0.13 mol) in anhydrous 1,4-dioxane (2 mL) was added dichlorozinc (23 mg, 0.17 mol) at 25° C. The mixture was stirred at 100° C. for 12 h. The reaction mixture was concentrated. The residue was purified by prep-HPLC to give title compound (10 mg). 1H NMR (400 MHz, CDCl3) δ 9.23 (s, 1H), 8.14 (s, 1H), 7.60 (d, J=8.8 Hz, 2H), 7.37-7.31 (m, 2H), 7.10 (m, 1H), 7.04-6.98 (m, 4H), 5.16 (s, 2H), 4.30-4.21 (m, 1H), 3.91 (q, J=8.8 Hz, 2H), 1.71 (s, 6H), 1.20 (d, J=6.4 Hz, 6H). LCMS (ESI): m/z, 518.3 [M+H]⁺.

### Example 203. Preparation of Compound E139

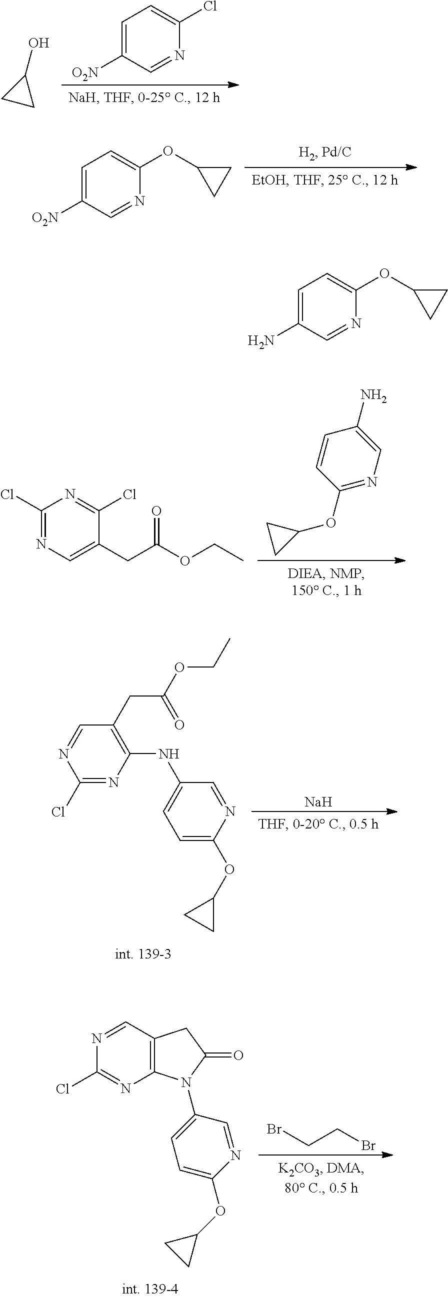

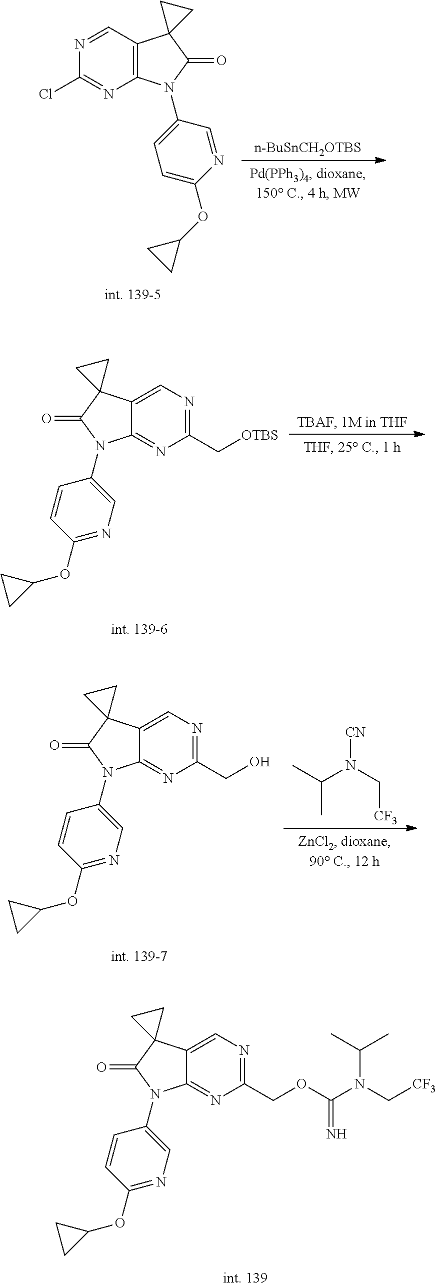

1. Synthesis of compound int. 139-1. To a solution of cyclopropanol (1.0 g, 17.2 mmol) in THF (50 mL) was added NaH (895 mg, 22.4 mmol, 60% purity) at 0° C., the mixture was stirred at 0° C. for 30 min. 2-chloro-5-nitro-pyridine (2.73 g, 17.2 mmol) was added to the mixture and stirred at 25° C. for 12 h. The reaction mixture was quenched by saturated NH₄Cl, diluted with water and extracted with EA. The combined organic phase was washed with saturated brine, dried over anhydrous Na₂SO₄, filtered and concentrated. The residue was purified by column chromatography to give title compound (1.8 g). ¹H NMR (400 MHz, CDCl3) δ 9.14 (d, J=2.8 Hz, 1H), 8.37 (dd, J=9.2, 2.8 Hz, 1H), 6.84 (d, J=9.2 Hz, 1H), 4.34-4.41 (m, 1H), 0.82-0.90 (m, 4H).

2. Synthesis of compound int. 139-2. To a solution of 2-cyclopropoxy-5-nitropyridine (1.8 g, 9.99 mmol) in EtOH (30 mL) and THF (20 mL) was added Pd/C (1.06 g, 1.00 mmol, 10% purity). The mixture was purged by H₂ and stirred at 25° C. for 12 h under H₂ atmosphere. The reaction mixture was filtered and concentrated. The crude product was purified by column chromatography to give title compound (1.4 g). LCMS (ESI): m/z, 151.0 [M+H]⁺. ¹H NMR (400 MHz, CDCl₃) δ 7.73 (d, J=2.8 Hz, 1H), 7.04 (dd, J=8.8, 2.8 Hz, I H), 6.64 (d, J=8.8 Hz, 1H), 4.09-4.03 (m, 1H), 0.78-0.72 (m, 4H).

3. Synthesis of compound int. 139-3. To a solution of ethyl 2-(2,4-dichloropyrimidin-5-yl)acetate (820 mg, 3.49 mmol) and 6-(cyclopropoxy)pyridin-3-amine (524 mg, 3.49 mmol) in NMP (20 mL) was added DIEA (1.15 mL) and the mixture was stirred at 150° C. in microwave for 1 h. Water was added and extracted with EtOAc. The combined organic phases were washed with brine, dried over anhydrous Na₂SO₄, filtered and concentrated. The residue was purified by column chromatography to give title compound (770 mg). 1H NMR (400 MHz, CDCl3) δ 8.40 (d, =2.4 Hz, 1H), 8.34 (s, 1H), 8.03 (s, 1H), 7.98 (dd, J=2.4, 0.8.8, 1H), 6.85 (d, J=8.8 Hz, 1H), 5.30 (s, 1H), 4.13-4.29 (m, 3H), 3.56 (s, 2H), 2.04 (s, 1H), 1.21-1.40 (m, 4H), 0.73-0.89 (m, 4H). LCMS (ESI): m/z, 349.1, 351.1 [M+H]⁺.

4. Synthesis of compound int. 139-4. To a solution of ethyl 2-[2-chloro-4-[[6-(cyclopropoxy)-3-pyridyl]amino]pyrimidin-5-yl]acetate (700 mg, 2.01 mmol) in THF (20 mL) was added NaH (241 mg, 6.02 mmol, 60% purity) at 0^{c}C and stirred at 20° C. for 30 min. The reaction was added water and extracted with EtOAc. The organic layers were concentrated. The residue was purified by column chromatography to give title compound (600 mg). LCMS (ESI): m/z, 303.0, 305.1 [M+H]⁺.

5. Synthesis of compound int. 139-5. To a solution of 2-chloro-7-(6-cyclopropoxypyridin-3-yl)-5,7-dihydro-6H-pyrrolo[2,3-d]pyrimidin-6-one (580 mg, 1.92 mmol) and K₂CO₃ (1.32 g, 9.58 mmol) in DMF (20 mL) was added 1,2-dibromoethane (1.26 g, 6.71 mmol) under N₂. The mixture reaction was stirred at 80° C. for 30 minutes. The reaction was added water and extracted with EtOAc. The organic layers were concentrated. The residue purified by column chromatography to give title compound (340 mg). LCMS (ESI): m/z, 329.1, 331.1 [M+H]⁺.

6. Synthesis of compound int. 139-6. To a mixture of 2′-chloro-7′-(6-cyclopropoxypyridin-3-yl)spiro[cyclopropane-1,5′-pyrrolo[2,3-d]pyrimidin]-6′(7′H)-one (0.1 g, 0.30 mol) in dioxane (4 mL) was added tert-butyl-dimethyl-(tributylstannylmethoxy)silane (146 mg, 0.33 mol) and Pd(PPh₃)₄ (35 mg, 0.030 mol). The mixture was purged by N₂ and stirred at 150° C. for 4 h under microwave. The reaction mixture was concentrated under reduced pressure. The residue was purified by prep-TLC to give title compound (35 mg). LCMS (ESI): m/z, 439.2 [M+H]⁺.

7. Synthesis of compound int. 139-7. To a mixture of 2′-(((tert-butyldimethylsilyl)oxy)methyl)-7′-(6-cyclopropoxypyridin-3-yl)spiro[cyclopropane-1,5′-pyrrolo[2,3-d]pyrimidin]-6′(7′H)-one (35 mg, 0.080 mol) in THF (1 mL) was added TBAF (1 M, 0.24 mL) at 25° C. The mixture was stirred at 25° C. for 1 h. The mixture was concentrated and purified by prep-TLC to obtain title compound (20 mg). LCMS (ESI): m/z, 325.1 [M+H]⁺.

8. Synthesis of compound E139. To a solution of 7′-(6-cyclopropoxypyridin-3-yl)-2′-(hydroxymethyl)spiro[cyclopropane-1,5′-pyrrolo[2,3-d]pyrimidin]-6′(7′H)-one (10 mg, 0.03 mol) and isopropyl(2,2,2-trifluoroethyl)cyanamide (15 mg, 0.092 mol) in anhydrous 1,4-dioxane (1 mL) was added dichlorozine (8.4 mg, 0.062 mmol) at 25° C. The mixture was stirred at 90° C. for 12 h. The reaction was concentrated and purified by prep-HPLC to give title compound (2.2 mg). LCMS (ESI): m/z, 491.2 [M+H]⁺.

### Example 204. Preparation of Compound E140

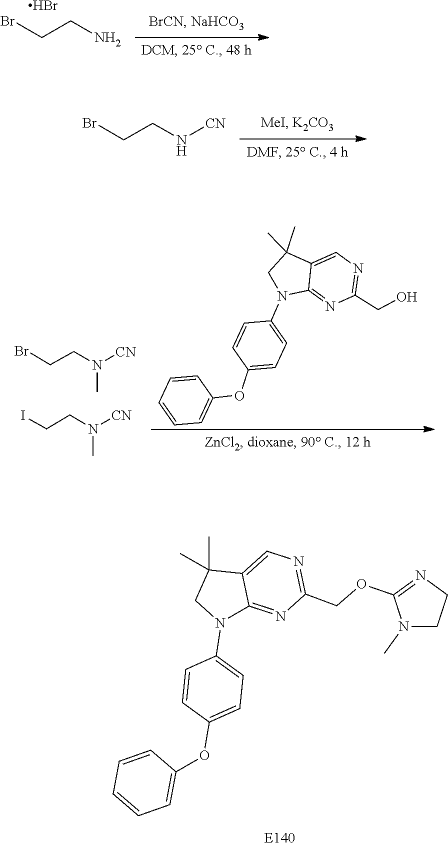

1. Synthesis of compound N-(2-bromoethyl)cyanamide. To a solution of 2-bromoethanamine (5.0 g, 24.4 mmol, HBr salt) in anhydrous DCM (35 mL) was added sat. aq. NaHCO₃ (64.4 g, 767 mmol, 35 mL), BrCN (5.17 g, 48.8 mmol) in DCM (35 mL) was added dropwise to the above mixture at 0° C. The mixture was stirred at 25° C. for 48 h. The reaction mixture was diluted with sat. aq. NaHCO₃ and extracted with DCM. The combined organic phase was washed with brine, dried over anhydrous Na₂SO₄, filtered and concentrated in vacuum. The residue was purified by column chromatography to give title compound (2.7 g). ¹H NMR (400 MHz, CDCl₃) δ 4.23 (br s, 1H), 3.56-3.52 (m, 2H), 3.51-3.45 (m, 2H).

2. Synthesis of compound N-(2-bromoethyl)-N-methylcyanamide. To a solution of N-(2-bromoethyl)cyanamide (1.0 g, 6.71 mmol) in anhydrous DMF (20.0 mL) was added iodomethane (1.14 g, 8.05 mmol, 501 μL) and K₂CO₃ (2.78 g, 20.14 mmol, 1.15 mL). The mixture was stirred at 25° C. for 4 h. The reaction mixture was quenched by saturated NH₄Cl and extracted with EA. The combined organic phase was washed with brine, dried over anhydrous Na₂SO₄, filtered and concentrated. The residue was purified by column chromatography to give title compound (0.27 g) and byproduct N-(2-iodoethyl)-N-methylcyanamide (0.35 g). ¹H NMR (400 MHz, CDCl3) δ 3.54-3.49 (m, 2H), 3.45-3.40 (m, 2H), 2.97 (s, 3H).

3. Synthesis of compound E140. To a solution of (5,5-dimethyl-7-(4-phenoxyphenyl)-6,7-dihydro-5H-pyrrolo[2,3-d]pyrimidin-2-yl)methanol (50 mg, 0.14 mmol) and N-(2-iodoethyl)-N-methylcyanamide (51 mg, 0.24 mmol) in anhydrous 1,4-dioxane (2 mL) was added dichlorozinc (59 mg, 0.43 mmol) at 25° C. The mixture was stirred at 90° C. for 12 h. The mixture was quenched by NaHCO₃ and extracted with EA. The combined organic phase was washed with brine, dried over Na₂SO₄, filtered and concentrated in vacuum. The residue was purified by prep-HPLC to give title compound (10.2 mg). ¹H NMR (400 MHz, CDCl3) δ 8.07 (s, 1H), 7.81 (s, 2H), 7.33 (m, 2H), 7.12-6.97 (m, 5H), 5.31 (s, 2H), 3.82 (s, 2H), 3.63 (m, 2H), 3.38 (m, 2H), 2.81 (s, 3H), 1.44 (s, 6H). LCMS (ESI): m/z, 430.2 [M+H]⁺.

### Example 205. Preparation of Compound E141

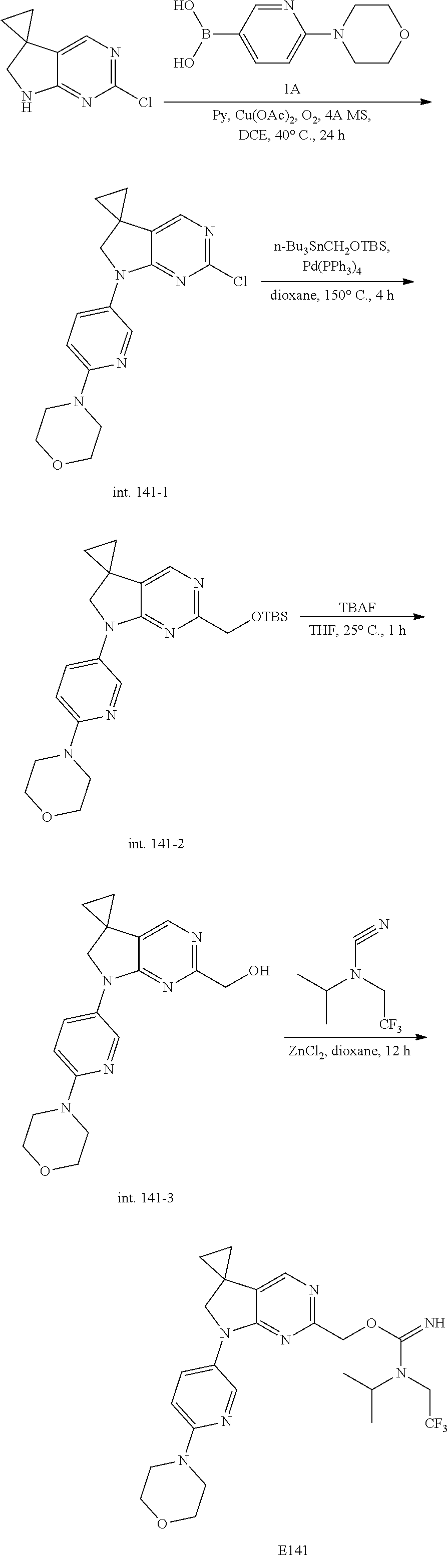

1. Synthesis of compound int. 141-1. To a solution of 2′-chloro-6′,7′-dihydrospiro[cyclopropane-1,5′-pyrrolo[2,3-d]pyrimidine](150 mg, 0.83 mmol) and (6-morpholino-3-pyridyl)boronic acid (515 mg, 2.48 mmol) in DCE (6 mL) was added Cu(AcO)₂ (180 mg, 0.99 mol), pyridine (0.2 mL) and 4 Å Molecular Sieve (100 mg) under O₂. The reaction was stirred at 40° C. for 24 h. The mixture was filtered and concentrated. The residue was purified by column chromatography to give title compound (0.11 g). ¹H NMR (400 MHz, CDCl3) δ 8.30 (d, J=2.4 Hz, 1H), 8.21 (dd, J=9.2, 2.8 Hz, 1H), 7.42 (s, 1H), 6.72 (m, 1H), 4.08 (s, 2H), 3.84-3.82 (m, 4H), 3.51-3.46 (m, 4H), 1.23-1.16 (m, 4H). LCMS (ESI): m/z, 344.1, 346.1 [M+H]⁺.

2. Synthesis of compound int. 141-2. To a solution of 4-(5-(2′-chlorospiro[cyclopropane-1,5′-pyrrolo[2,3-d]pyrimidin]-7′(6′H)-yl)pyridin-2-yl)morpholine (0.2 g, 0.58 mmol) in dioxane (6 mL) was added tert-butyl-dimethyl-(tributylstannylmethoxy)silane (329 mg, 0.76 mmol) and Pd(PPh₃)₄ (67 mg, 0.058 mmol). The mixture was purged by N₂ and stirred at 150° C. for 4 h under microwave. The reaction mixture was concentrated under reduced pressure to give a residue and purified by column chromatography to give title compound (0.3 g). LCMS (ESI): m/z, 454.3 [M+H]⁺.

3. Synthesis of compound int.141-3. To a mixture of 4-(5-(2′-(((tert-butyldimethylsilyl)oxy)methyl)spiro[cyclopropane-1,5′-pyrrolo[2,3-d]pyrimidin]-7′(6′H)-yl)pyridin-2-yl)morpholine (0.3 g, 0.66 mmol) in THF (6 mL) was added TBAF (1 M, 1.98 mL) at 25° C. The mixture was stirred at 25° C. for 1 h. The reaction mixture was concentrated and the residue was purified by prep-TLC to give title compound (0.1 g). LCMS (ESI): m/z, 340.2 [M+H]⁺.

4. Synthesis of compound E141. To a mixture of (7′-(6-morpholinopyridin-3-yl)-6′,7′-dihydrospiro[cyclopropane-1,5′-pyrrolo[2,3-d]pyrimidin]-2′-yl)methanol (90 mg, 0.26 mmol) and isopropyl(2,2,2-trifluoroethyl)cyanamide (132 mg, 0.79 mmol) in anhydrous 1,4-dioxane (2.60 mL) was added dichlorozine (1 M, 0.80 mL) at 25° C. The mixture was stirred at 90° C. for 12 h. The reaction mixture was concentrated and the residue was purified by prep-HPLC to give title compound (17 mg). ¹H NMR (400 MHz, CDCl3) δ 8.60 (d, J=2.8 Hz, 1H), 8.28 (d, J=9.2 Hz, 1H), 7.65 (s, 1H), 7.01 (d, J=9.2 Hz, 1H), 5.46 (s, 2H), 4.84 (s, 2H), 4.46 (s, 2H), 4.13 (s, 2H), 3.86 (m, 4H), 3.62 (m, 3H), 1.30 (s, 2H), 1.28 (s, 2H), 1.25 (d, J=6.8 Hz, 6H). LCMS (ESI): m/z, 506.3 [M+H]⁺.

### Example 206. Preparation of Compound E142

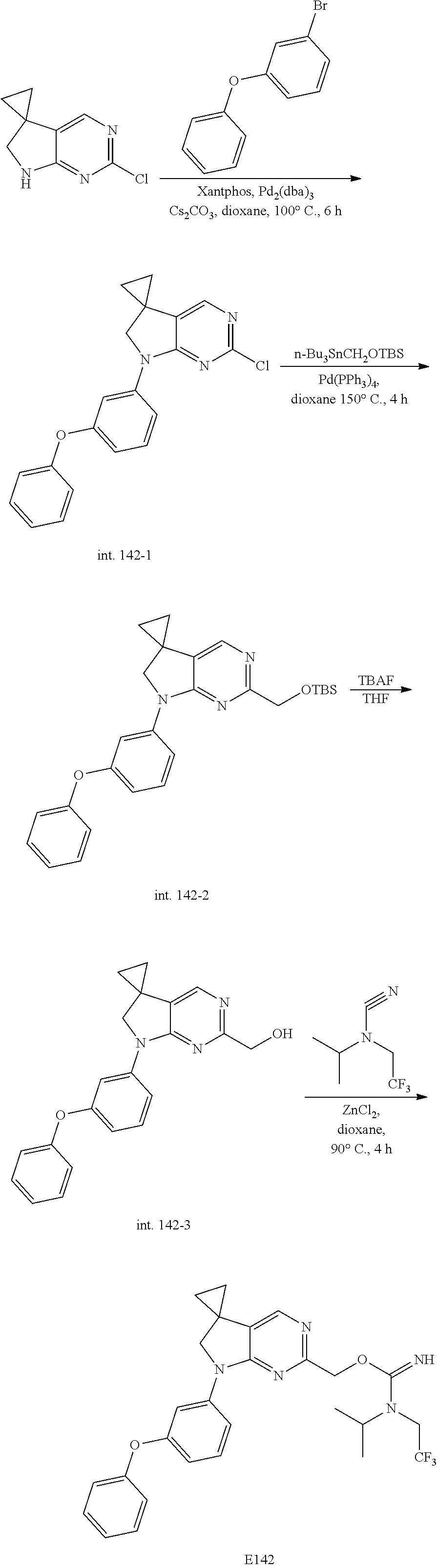

1. Synthesis of compound int. 142-1. A mixture of 2-chlorospiro[6,7-dihydropyrrolo[2,3-d]pyrimidine-5,1′-cyclopropane](250 mg, 1.38 mmol), 1-bromo-3-phenoxy-benzene (686 mg, 2.75 mmol), XantPhos (239 mg, 0.413 mmol), Pd₂(dba)₃ (189 mg, 0.206 mmol), Cs₂CO₃ (897 mg, 2.75 mmol) in dioxane (10 mL) was stirred at 100° C. for 6 hours. The mixture was concentrated. The residue was purified by column chromatography to afford title compound (250 mg).

2. Synthesis of compound int. 142-2. To a solution of 2-chloro-7-(3-phenoxyphenyl)spiro[6H-pyrrolo[2,3-d]pyrimidine-5,1′-cyclopropane](300 mg, 0.857 mmol) in dioxane (8 mL) was added tert-butyl-dimethyl-(tributylstannylmethoxy)silane (560. mg, 1.29 mmol) and Pd(PPh₃)₄ (198 mg, 0.172 mmol). The mixture was stirred at 150° C. for 4 hours under N₂ atmosphere. The mixture was concentrated. The residue was purified by column chromatography to afford title compound (150 mg).

3. Synthesis of compound int.142-3. To a solution of tert-butyl-dimethyl-[[7-(3-phenoxyphenyl)spiro[6H-pyrrolo[2,3-d]pyrimidine-5,1′-cyclopropane]-2-yl]methoxy]silane (150 mg, 0.327 mmol) in THF (5 mL) was added TBAF (1 M, 0.98 mL). The mixture was stirred at 25° C. for 2 hours. The mixture was concentrated. The residue was purified by column chromatography to afford title compound (100 mg).

4. Synthesis of compound E142. To a solution of [7-(3-phenoxyphenyl)spiro[6H-pyrrolo[2,3-d]pyrimidine-5,1′-cyclopropane]-2-yl]methanol (105 mg, 0.31 mmol), isopropyl(2,2,2-trifluoroethyl)cyanamide (151 mg, 0.91 mmol) in dioxane (8 mL) was added ZnCl₂ (331 mg, 2.43 mmol). The mixture was stirred at 90° C. for 4 hours. The mixture was filtered. The filtrate was purified by pre-HPLC to afford title compound (30 mg). ¹H NMR (400 MHz, DMSO-d6) δ 9.41 (brs, 1H), 7.87 (s, 1H), 7.69 (s, 1H), 7.52 (d, J=8.0 Hz, 1H), 7.43-7.37 (m, 3H), 7.18-7.14 (m, 1H), 7.05 (d, J=8.0 Hz, 2H), 6.70 (d, J=8.4 Hz, 1H), 5.45 (s, 2H), 4.45-4.36 (m, 2H), 4.26-4.20 (m, 1H), 4.15 (s, 2H), 1.23-1.21 (m, 10H). LCMS (ESI): m/z, 512.3 [M+H]⁺.

### Example 207. Preparation of Compound E143

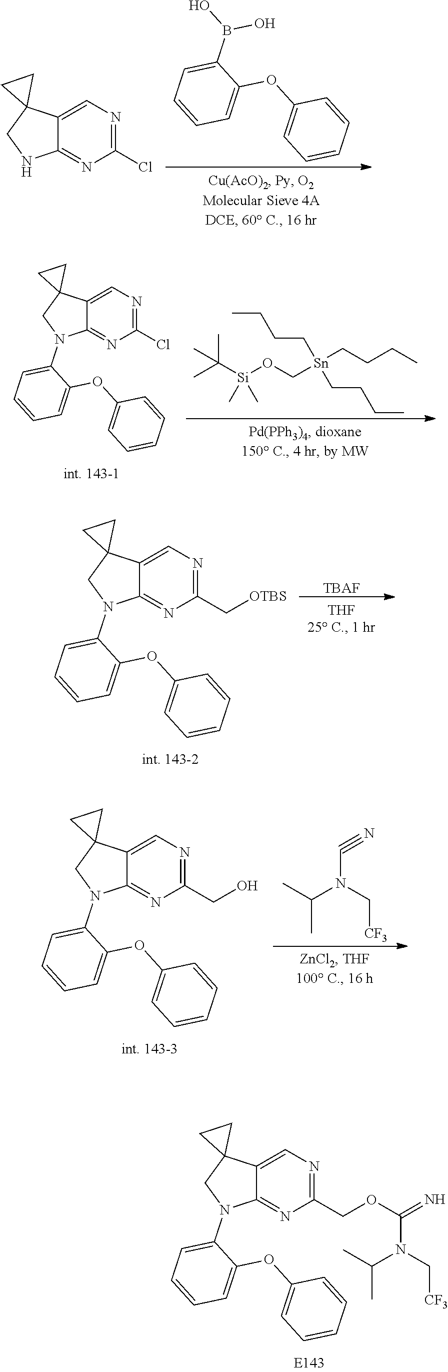

1. Synthesis of compound int. 143-1. To a solution of 2′-chloro-6′,7′-dihydrospiro[cyclopropane-1,5′-pyrrolo[2,3-d]pyrimidine](200 mg, 1.10 mmol) and (2-phenoxyphenyl)boronic acid (472 mg, 2.20 mmol) in DCE (5 mL) was added Cu(AcO)₂ (240 mg, 1.32 mmol) and 4 Å molecular sieve (200 mg) under O₂. The reaction was stirred at 20° C. for 48 hours. The reaction was concentrated to give a residue. The crude product was purified by column chromatography to give title compound (142 mg).

2. Synthesis of compound int. 143-2. To a solution of 2′-chloro-7′-(2-phenoxyphenyl)-6′,7′-dihydrospiro[cyclopropane-1,5′-pyrrolo[2,3-d]pyrimidine](60 mg, 0.17 mmol) and tert-butyldimethyl((tributylstannyl)methoxy)silane (82 mg, 0.19 mmol) in dioxane (3 mL) was added Pd(PPh₃)₄ (59 mg, 0.051 mmol) and stirred at 150° C. for 4 hours by microwave. The reaction was filtered and the filtrate was concentrated to give a residue which was purified by column chromatography to give title compound (8 mg).

3. Synthesis of compound int.143-3. To a mixture of 2′-(((tert-butyldimethylsilyl)oxy)methyl)-7′-(2-phenoxyphenyl)-6′,7′-dihydrospiro[cyclopropane-1,5′-pyrrolo[2,3-d]pyrimidine](48 mg, 0.1 mmol) in THF (2 mL) was added TBAF (1 M, 0.32 mL) at 25° C. The mixture was stirred at 25° C. for 1 h. The reaction was filtered and the filtrate was concentrated. The residue which was purified by column chromatography to give title compound (36 mg).

4. Synthesis of compound E143. To a solution of (7′-(2-phenoxyphenyl)-6′,7′-dihydrospiro[cyclopropane-1,5′-pyrrolo[2,3-d]pyrimidin]-2′-yl)methanol (36 mg, 0.1 mmol) and N-isopropyl-N-(2,2,2-trifluoroethyl)cyanamide (52 mg, 0.31 mmol) in dioxane (3 mL) was added ZnCl₂ (114 mg, 0.83 mmol) and stirred at 90° C. for 6 h. The reaction was filtered and the filtrate was purified by Prep-HPLC to give title compound (8 mg). ¹H NMR (400 MHz, CDCl₃) (δ 7.52 (s, 1H), 7.47 (dd, J=8.0, 1.6 Hz, 1H), 7.37-7.27 (m, 3H), 7.23 (dd, J=7.6, 1.2 Hz, 1H), 7.12-7.01 (m, 2H), 6.94-6.85 (m, 2H), 5.43 (s, 2H), 4.44-4.29 (m, 1H), 4.11 (s, 2H), 4.01 (q, J=8.4 Hz, 2H), 1.26-1.18 (m, 8H), 1.16-1.08 (m, 2H). LCMS (ESI): m/z 512.3 [M+H]1.

### Example 208. Preparation of Compound E144

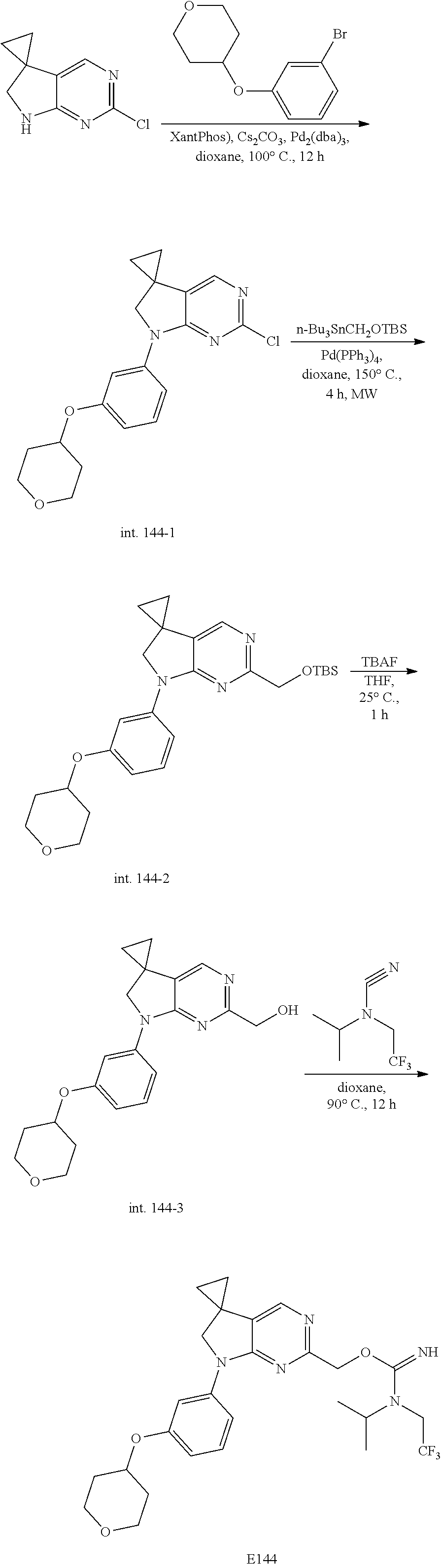

1. Synthesis of compound int. 144-1. To a solution of 2′-chloro-6′,7′-dihydrospiro[cyclopropane-1,5′-pyrrolo[2,3-d]pyrimidine](0.25 g, 1.38 mmol), 4-(3-bromophenoxy)tetrahydropyran (708 mg, 2.75 mmol), XantPhos (159 mg, 0.28 mmol), Cs₂CO₃ (897 mg, 2.75 mmol), Pd₂(dba)₃ (126 mg, 0.14 mmol) in Dioxane (8 mL) was stirred at 100° C. for 4 hours under N₂ atmosphere. The reaction was quenched by saturate NH₄Cl and extracted with EA. The combined organic phase was washed by brine, dried over anhydrous Na₂SO₄, filtered and concentrated. The residue was purified by column chromatography to give title compound (0.4 g). LCMS (ESI): m/z, 358.2, 360.2 [M+H]+.

2. Synthesis of compound int. 144-2. To a solution of 2′-chloro-7′-(3-((tetrahydro-2H-pyran-4-yl)oxy)phenyl)-6′,7′-dihydrospiro[cyclopropane-1,5′-pyrrolo[2,3-d]pyrimidine](0.4 g, 1.12 mmol) in dioxane (8 mL) was added tert-butyl-dimethyl-(tributylstannylmethoxy)silane (730 mg, 1.68 mmol) and Pd(PPh₃)₄ (258 mg, 0.22 mmol). The mixture was purged by N₂ and stirred at 150° C. for 4 h under microwave. The reaction was concentrated to give a residue which was purified by column chromatography to give title compound (0.15 g). LCMS (ESI): m/z, 468.3 [M+H]⁺.

3. Synthesis of compound int.144-3. To a mixture of 2′-(((tert-butyldimethylsilyl)oxy)methyl)-7′-(3-((tetrahydro-2H-pyran-4-yl)oxy)phenyl)-6′,7′-dihydrospiro[cyclopropane-1,5′-pyrrolo[2,3-d]pyrimidine](150 mg, 0.32 mmol) in THF (4 mL) was added TBAF (1 M, 0.96 mL) at 25° C. The mixture was stirred at 25° C. for 1 h. The reaction mixture was concentrated and the residue was purified by prep-TLC to give title compound (0.1 g). LCMS (ESI): m/z, 354.2 [M+H]⁺.

4. Synthesis of compound E144. To a mixture of (7′-(3-((tetrahydro-2H-pyran-4-yl)oxy)phenyl)-6′,7′-dihydrospiro[cyclopropane-1,5′-pyrrolo[2,3-d]pyrimidin]-2′-yl)methanol (100 mg, 0.28 mmol) and isopropyl(2,2,2-trifluoroethyl)cyanamide (141 mg, 0.85 mmol) in anhydrous 1,4-dioxane (6 mL) was added dichlorozinc (1 M, 0.85 mL) at 25° C. The mixture was stirred at 90° C. for 4 h. The reaction mixture was concentrated and purified by prep-HPLC to give title compound (17 mg). ¹H NMR (400 MHz, CDCl3) δ 7.66 (s, 1H), 7.37-7.30 (m, 2H), 7.28 (d, J=2.4 Hz, 1H), 6.77 (m, 1H), 5.46 (s, 2H), 4.52 (m, 1H), 4.18 (s, 2H), 4.10 (m, 2H), 4.02-3.97 (m, 2H), 3.88 (m, 1H), 3.59 (m, 2H), 2.08-2.03 (m, 2H), 1.82 (m, 2H), 1.29 (m, 6H), 1.26-1.24 (m, 4H). LCMS (ESI): m/z, 520.3 [M+H]⁺.

### Example 209. Preparation of Compound E145

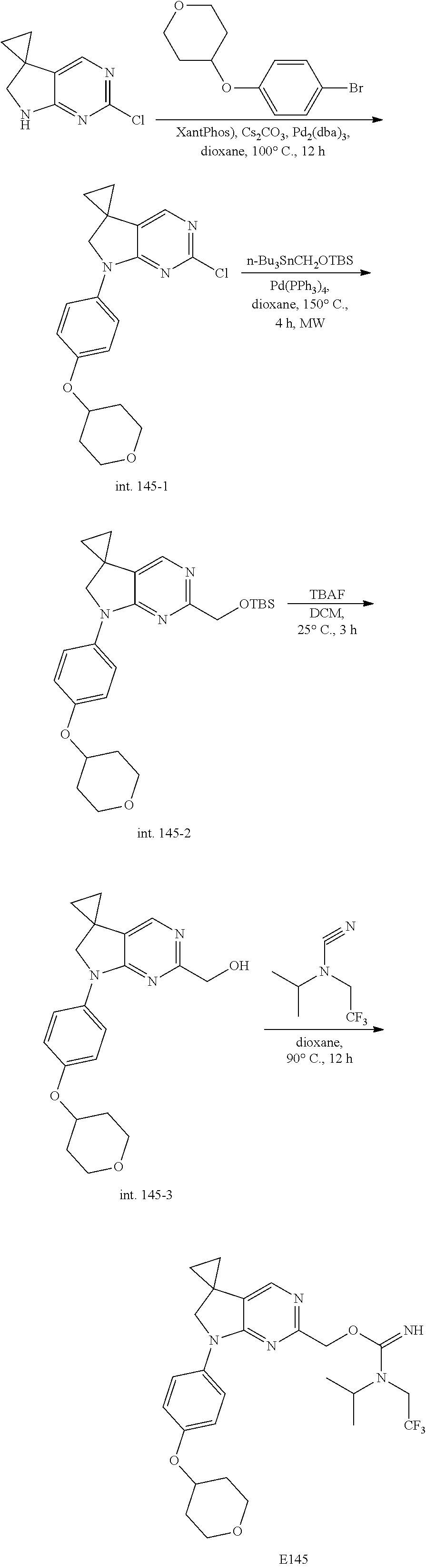

1. Synthesis of compound int. 145-1. A mixture of 2-chlorospiro[6,7-dihydropyrrolo[2,3-d]pyrimidine-5,1′-cyclopropane](170 mg, 0.94 mmol), 4-(4-bromophenoxy)tetrahydropyran (481 mg, 1.87 mmol), XantPhos (108 mg, 0.19 mmol), Pd₂(dba)₃ (86 mg, 0.094 mmol), Cs₂CO₃ (610 mg, 1.87 mmol) in dioxane (5 mL) was stirred at 100° C. for 3 h under N₂. The reaction was added water and extracted with EtOAc. The organic layers were concentrated. The residue was purified by column chromatography to give title compound (150 mg). LCMS (ESI):m/z, 358.1, 360.1 [M+H]⁺.

2. Synthesis of compound int. 145-2. To a solution of 2′-chloro-7′-(4-((tetrahydro-2H-pyran-4-yl)oxy)phenyl)-6′,7′-dihydrospiro[cyclopropane-1,5′-pyrrolo[2,3-d]pyrimidine](133 mg, 0.37 mmol) and tert-butyl-dimethyl-(tributylstannylmethoxy)silane (243 mg, 0.56 mmol) in dioxane (3 mL) was added Pd(PPh₃)₄ (172 mg, 0.15 mmol) and stirred at 150° C. for 4 h by microwave. The reaction was concentrated under reduce pressure to give a residue. The residue was purified by column chromatography to give title compound (100 mg).

3. Synthesis of compound int.145-3. To a solution of tert-butyl-dimethyl-[[7-(4-tetrahydropyran-4-yloxyphenyl)spiro[6H-pyrrolo[2,3-d]pyrimidine-5,1′-cyclopropane]-2-yl]methoxy]silane (80 mg, 0.17 mmol) in DCM (2 mL) was added TBAF (1 M in THF) (1 M, 0.86 mL). The mixture was stirred at 25° C. for 3 h. The reaction was added water and extracted with EtOAc. The organic layers were washed with brine and concentrated. The residue was purified by column chromatography to give title compound (55 mg). LCMS (ESI): m/z, 354.2 [M+H]⁺.

4. Synthesis of compound E145. To a solution of (7′-(4-((tetrahydro-2H-pyran-4-yl)oxy)phenyl)-6′,7′-dihydrospiro[cyclopropane-1,5′-pyrrolo[2,3-d]pyrimidin]-2′-yl)methanol (50 mg, 0.14 mmol) and isopropyl(2,2,2-trifluoroethyl)cyanamide (71 mg, 0.42 mmol) in anhydrous 1,4-dioxane (0.6 mL) was added dichlorozinc (0.4 mL, 1 M in THF) at 25° C. under N₂. The mixture was stirred at 90° C. for 12 h. The reaction was added water and extracted with EtOAc. The organic layers were concentrated. The residue was purified by Prep-HPLC to give title compound (12 mg). ¹H NMR (400 MHz, CDCl₃) δ 7.60-7.42 (m, 3H), 7.01-6.97 (m, 2H), 5.44 (s, 2H), 4.55-4.25 (m, 2H), 4.25-3.80 (m, 6H), 3.62-3.56 (m, 2H), 2.07-2.00 (m, 2H), 1.85-1.75 (m, 2H), 1.35-1.20 (m, 10H). LCMS (ESI): m/z, 520.2 [M+H]⁺.

### Example 210. Preparation of Compound E146

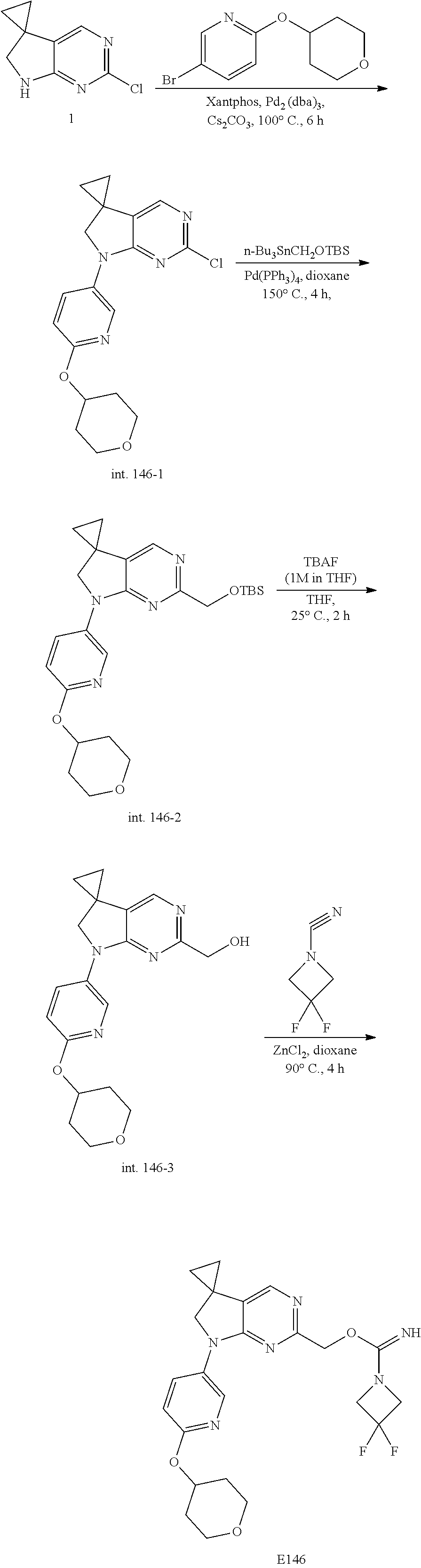

1. Synthesis of compound int. 146-1. A mixture of 2-chlorospiro[6,7-dihydropyrrolo[2,3-d]pyrimidine-5,1′-cyclopropane](520 mg, 2.86 mmol), 5-bromo-2-tetrahydropyran-4-yloxy-pyridine (1.48 g, 5.73 mmol), XantPhos (331 mg, 0.573 mmol), Cs₂CO₃ (1.87 g, 5.73 mmol), pd₂(dba)₃ (262 mg, 0.286 mmol) in Dioxane (20 mL) was stirred at 100° C. for 6 hours under N₂ atmosphere. The mixture was concentrated. The residue was purified by column chromatography to afford title compound (780 mg). 1H NMR (400 MHz, DMSO-d6) δ 8.41 (d, J=2.8 Hz, 1H), 8.17 (dd, J=3.2, 9.2 Hz, ¹H), 7.69 (s, 1H), 6.91 (d, J=8.8 Hz, 1H), 5.19-5.12 (m, 1H), 4.16 (s, 2H), 3.89-3.84 (m, 2H), 3.52-3.46 (m, 2H), 2.03-1.97 (m, 2H), 1.67-1.58 (m, 2H), 1.25-1.16 (m, 4H). LCMS (EST):m/z, 359.1 361.1 [M+H]⁺

2. Synthesis of compound int. 146-2. A mixture of 2′-chloro-6′,7′-dihydrospiro[cyclopropane-1,5′-pyrrolo[2,3-d]pyrimidine](750 mg, 2.09 mmol), tert-butyl-dimethyl-(tributylstannylmethoxy)silane (1.18 g, 2.72 mmol), Pd(Ph₃)₄ (483 mg, 0.418 mmol) in Dioxane (12 mL) was stirred at 150° C. for 4 hours under N₂ atmosphere and microwave The mixture was concentrated. The residue was purified by column chromatography to afford title compound (287 mg). LCMS (ESI): m/z, 469.3 [M+H]⁺.

3. Synthesis of compound int.146-3. To a solution of 2′-(((tert-butyldimethylsilyl)oxy)methyl)-7′-(6-((tetrahydro-2H-pyran-4-yl)oxy)pyridin-3-yl)-6′,7′-dihydrospiro[cyclopropane-1,5′-pyrrolo[2,3-d]pyrimidine](287 mg, 0.612 mmol) in THF (8 mL) was added TBAF (1 M, 1.84 mL).The mixture was stirred at 25° C. for 2 hours. The mixture was concentrated. The residue was purified by column chromatography to afford title compound (200 mg). LCMS (ESI): m/z, 355.2 [M+H]+.

4. Synthesis of compound E146. To a solution of (7′-(6-((tetrahydro-2H-pyran-4-yl)oxy)pyridin-3-yl)-6′,7′-dihydrospiro[cyclopropane-1,5′-pyrrolo[2,3-d]pyrimidin]-2′-yl)methanol (70 mg, 0.197 mmol), 3,3-difluoroazetidine-1-carbonitrile (70 mg, 0.592 mmol) in Dioxane (6 mL) was added ZnCl₂ (215 mg, 1.58 mmol). The mixture was stirred at 90° C. for 4 hours under N₂ atmosphere. The mixture was filtered. The filtrated was purified by pre-HPLC to afford title compound (52 mg). 1H NMR (400 MHz, DMSO-d6) δ 9.47 (brs, 1H), 8.62 (d, J=2.8 Hz, 1H), 8.13 (dd, J=2.8, 8.8 Hz, 1H), 7.85 (s, 1H), 6.90 (d, J=8.8 Hz, 1H), 5.43 (s, 2H), 5.19-5.12 (m, 1H), 4.72 (d, J=11.6 Hz, 4H), 4.15 (s, 2H), 3.90-3.85 (m, 2H), 3.51-3.45 (m, 2H), 2.07-1.97 (m, 2H), 1.67-1.58 (m, 2H), 1.31-1.18 (m, 4H). LCMS (ESI): m/z, 473.2 [M+H]⁺.

### Example 211. Preparation of Compound E147

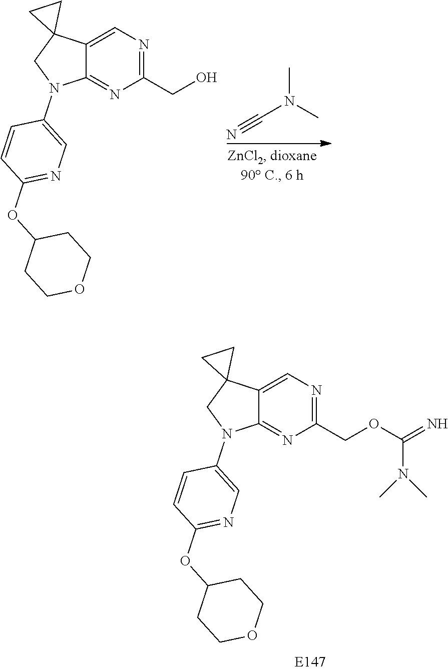

To a solution of (7′-(6-((tetrahydro-2H-pyran-4-yl)oxy)pyridin-3-yl)-6′,7′-dihydrospiro[cyclopropane-1,5′-pyrrolo[2,3-d]pyrimidin]-2′-yl)methanol (60 mg, 0.17 mmol), dimethylcyanamide (36 mg, 0.51 mmol) in dioxane (5 mL) was added ZnCl₂ (184 mg, 1.35 mmol). The mixture was stirred at 90° C. for 6 hours. The mixture was filtered. The filtrate was purified by prep-HPLC to afford title compound (52 mg). ¹H NMR (400 MHz, DMSO-d₆) δ 8.80 (s, 1H), 8.54 (d, J=2.8 Hz, 1H), 8.13 (dd, J=3.2, 9.2 Hz, 1H), 7.84 (s, 1H), 6.90 (d, J=8.8 Hz, 1H), 5.45 (s, 2H), 5.18-5.11 (m, 11H), 4.14 (s, 2H), 3.90-3.85 (m, 2H), 3.53-3.50 (m, 2H), 3.09 (s, 6H), 2.03-1.97 (m, 2H), 1.68-1.59 (m, 2H), 1.25-1.18 (m, 4H). LCMS (ESI): m/z, 425.2 [M+H]⁺.

### Example 212. Preparation of Compound E148

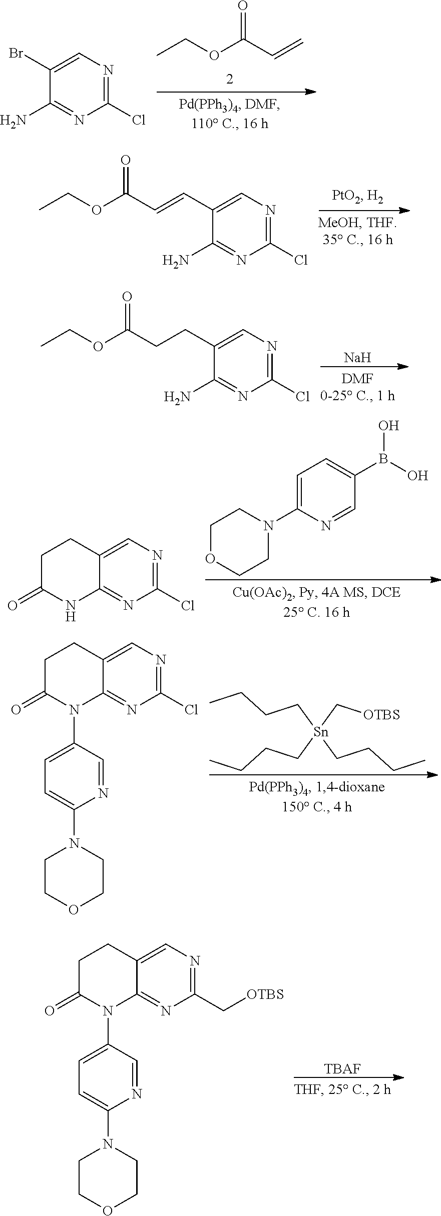

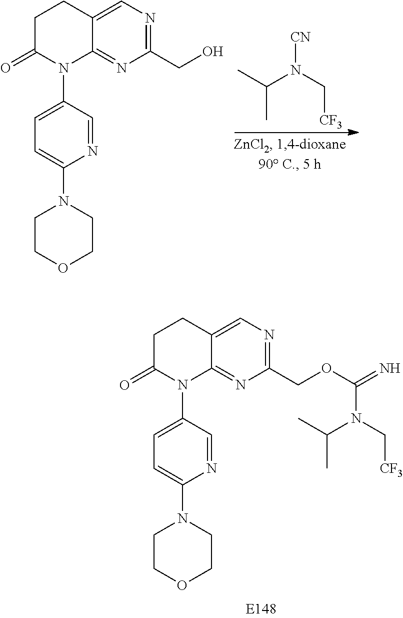

1. Synthesis of compound ethyl (E)-3-(4-amino-2-chloro-pyrimidin-5-yl)prop-2-enoate. To a solution of 5-bromo-2-chloro-pyrimidin-4-amine (12.0 g, 57.57 mmol) and ethyl prop-2-enoate (28.8 g, 288 mmol, 15.7 mL) in DMF (120 mL) was added Pd(PPh₃)₄ (6.65 g, 5.76 mmol) under N₂. The reaction was stirred at 110° C. for 16 h. The solvent was removed to yield a residue which was purified by prep-HPLC to afford title compound (2.1 g). ¹H NMR (400 MHz, CDCl₃) δ 8.29 (s, 1H), 7.52 (dd, J=16.0, 0.8 Hz, 1H), 6.41 (d, J=16.0 Hz, 1H), 5.70 (s, 2H), 4.28 (q, J=7.2 Hz, 2H), 1.34 (t, J=7.2 Hz, 3H). LCMS (ESI): m/z, 228.0 [M+H]⁺.

2. Synthesis of compound ethyl 3-(4-amino-2-chloro-pyrimidin-5-yl)propanoate. To a solution of ethyl (E)-3-(4-amino-2-chloro-pyrimidin-5-yl)prop-2-enoate (2.1 g, 9.22 mmol) in MeOH (15 mL) and THF (15 mL) was added PtO₂ (394 mg, 1.73 mmol). The suspension was degassed under vacuum and purged with H₂ 3 times. The mixture was stirred under H₂ (15 psi) at 35° C. for 16 h. The mixture was filtered and the filter cake was washed with THF. The filtrate was concentrated to afford title compound (2.0 g).

3. Synthesis of compound 2-chloro-6,8-dihydro-5H-pyrido[2,3-d]pyrimidin-7-one. To a solution of ethyl 3-(4-amino-2-chloro-pyrimidin-5-yl)propanoate (1.8 g, 7.84 mmol) in DMF (20 mL) was added NaH (627 mg, 15.68 mmol, 60% purity) at 0° C. and stirred at 25° C. for 1 h. The mixture was quenched by sat.aq. NH₄Cl at 0° C. and treated with EtOAc. The mixture was filtered and the filtrate was extracted with EtOAc. The combined organic phases were washed with brine, dried over anhydrous Na₂SO₄, filtered and concentrated to yield a residue. The filter cake was washed with THF and the filtrated was concentrated to afford title compound (1.4 g). ¹H NMR (400 MHz, CDCl₃) δ 8.29 (s, 1H), 7.98 (s, 1H), 2.98 (dd, J=8.4, 6.8 Hz, 2H), 2.75 (dd, J=8.5, 6.8 Hz, 2H).

4. Synthesis of compound 2-chloro-8-(6-morpholino-3-pyridyl)-5,6-dihydropyrido[2,3-d]pyrimidin-7-one. To a solution of 2-chloro-6,8-dihydro-5H-pyrido[2,3-d]pyrimidin-7-one (500 mg, 2.72 mmol) and (6-morpholino-3-pyridyl)boronic acid (1.13 g, 5.45 mmol) in DCE (15 mL) were added Cu(OAc)₂ (594 mg, 3.27 mmol), Py (0.66 mL) and 4 Å MS (800 mg). The mixture was stirred at 25° C. for 16 h under O₂. The mixture was filtered and the filter cake was washed with DCM. The filtrate was concentrated to yield a residue which was purified by column chromatography to afford title compound (510 mg). ¹H NMR (400 MHz, CDCl₃) δ 7.98 (d, J=2.4 Hz, 1H), 7.31 (dd, J=9.2, 2.6 Hz, 1H), 6.73 (d, J=9.2 Hz, 1H), 3.89-3.78 (m, 4H), 3.63-3.53 (m, 4H), 3.05 (dd, J=8.8, 6.0 Hz, 2H), 3.00-2.88 (m, 2H). LCMS (ESI): m/z, 346.1 [M+H]⁺.

5. Synthesis of compound 2-[[tert-butyl(dimethyl)silyl]oxymethyl]-8-(6-morpholino-3-pyridyl)-5,6-dihydropyrido[2,3-d]pyrimidin-7-one. To a solution of 2-chloro-8-(6-morpholino-3-pyridyl)-5,6-dihydropyrido[2,3-d]pyrimidin-7-one (530 mg, 1.53 mmol) and tert-butyl-dimethyl-(tributylstannylmethoxy)silane (800 mg, 1.84 mmol) in 1,4-dioxane (10 mL) was added Pd(PPh₃)₄ (354 mg, 0.31 mmol). The mixture was stirred at 150° C. under N₂ in microwave for 4 h. The mixture was concentrated to yield a residue which was purified by column chromatography to afford title compound (250 mg).

6. Synthesis of compound 2-(hydroxymethyl)-8-(6-morpholino-3-pyridyl)-5,6-dihydropyrido[2,3-d]pyrimidin-7-one. To a solution of 2-[[tert-butyl(dimethyl)silyl]oxymethyl]-8-(6-morpholino-3-pyridyl)-5,6-dihydropyrido[2,3-d]pyrimidin-7-one (250 mg, 0.55 mmol) in THF (2 mL) was added TBAF (1 M, 1.65 mL) and the mixture was stirred at 25° C. for 2 h. The reaction was diluted with water, extracted with EtOAc. The combined organic phases were washed with brine, dried over anhydrous Na₂SO₄, filtered and concentrated was purified by prep-TLC to afford title compound (50 mg). ¹H NMR (400 MHz, CDCl₃) δ 7.98 (d, J 2.4 Hz, 1H), 7.31 (dd, J 9.2, 2.8 Hz, 1H), 6.72 (d, J=8.8 Hz, 1H), 4.58 (s, 2H), 3.90-3.77 (m, 4H), 3.67-3.51 (m, 4H), 3.07 (dd, J=8.8, 6.3 Hz, 2H), 3.02-2.88 (m, 2H). LCMS (ESI): m/z, 342.2 [M+H]⁺.

7. Synthesis of compound 1-isopropyl-2-[[8-(6-morpholino-3-pyridyl)-7-oxo-5,6-dihydropyrido[2,3-d]pyrimidin-2-yl]methyl]-1-(2,2,2-trifluoroethyl)isourea. To a solution of 2-(hydroxymethyl)-8-(6-morpholino-3-pyridyl)-5,6-dihydropyrido[2,3-d]pyrimidin-7-one (30 mg, 0.088 mmol) and isopropyl(2,2,2-trifluoroethyl)cyanamide (43.8 mg, 0.26 mmol) in 1,4-Dioxane (4 mL) was added ZnCl₂ (47.9 mg, 0.35 mmol). The mixture was stirred at 90° C. for 5 h under N₂. The mixture was filtered which was purified by prep-HPLC to afford title compound (2.4 mg). ¹H NMR (400 MHz, CDCl₃) δ 8.43 (s, I H), 7.95 (d, J=2.4 Hz, 1H), 7.30 (dd, J=9.2, 2.8 Hz, I H), 6.74 (d, J=9.2 Hz, 1H), 5.27 (d, J=3.2 Hz, 2H), 4.22-4.07 (m, I H), 3.89-3.79 (m, 4H), 3.72 (q, J=8.8, 7.2 Hz, 2H), 3.58 (dd, J=6.0, 4.0 Hz, 4H), 3.05 (dd, J=8.8, 6.0 Hz, 2H), 2.97-2.85 (m, 2H), 1.17 (d, J=6.8 Hz, 6H). LCMS (ESI): m/z, 508.2 [M+H]⁺.

### Example 213. Preparation of Compound E149

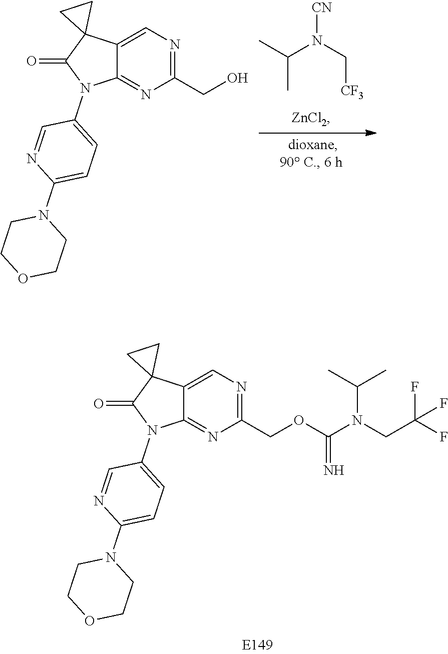

Compound E149 was synthesized as compound E147. ¹H NMR (400 MHz, CDCl₃) δ 8.35 (d, J=2.8 Hz, 1H), 8.06 (s, 1H), 7.64 (dd, J=9.2, 2.8 Hz, 1H), 6.73 (d, J=9.2 Hz, 1H), 5.25 (s, 2H), 4.19-4.01 (m, 1H), 3.89-3.79 (m, 6H), 3.61-3.54 (m, 4H), 1.99-1.94 (m, 2H), 1.79-1.75 (m, 2H), 1.18 (d, J=6.8 Hz, 6H). LCMS (ESI): m/z, 520.2 [M+H]⁺.

### Example 214. Preparation of Compound E150

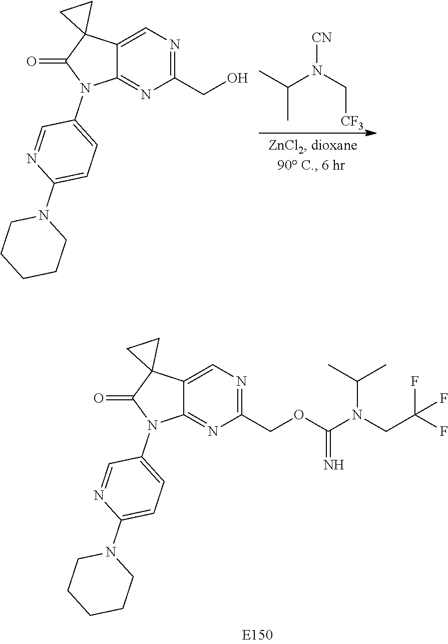

Compound E150 was synthesized as compound E147. ¹H NMR (400 MHz, CDCl₃) δ 8.34 (d, J=2.8 Hz, 1H), 8.06 (s, 1H), 7.63 (dd, J=9.2, 2.8 Hz, 1H), 6.72 (d, J 9.2 Hz, 1H), 5.25 (s, 2H), 4.71 (s, 1H), 4.12-4.01 (m, 2H), 3.85-3.83 (m, 4H), 3.64-3.54 (m, 4H), 2.02-1.93 (m, 2H), 1.81-1.73 (m, 2H), 1.25 (d, J=6.8 Hz, 9H), 1.17 (d, J=6.8 Hz, 6H). LCMS (ESI): m/z, 518.3 [M+H].

### Example 215. Preparation of Compound E151

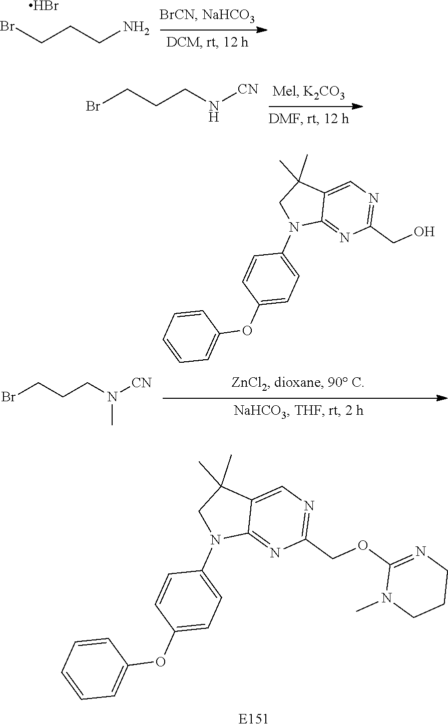

1. Synthesis of compound N-(3-bromopropyl)cyanamide. To a solution of 3-bromopropan-1-amine hydrobromide (2.0 g, 9.14 mmol) in anhydrous DCM (15 mL) and aq. NaHCO₃ (27.60 g, 328.53 mmol, 15 mL) was added dropwise a solution of carbononitridic bromide (1.94 g, 18.3 mmol) in DCM (15 mL) at 0° C. The mixture was stirred at 25° C. for 12 h. The reaction mixture was diluted with saturated NaHCO₃ and extracted with DCM. The combined organic phase was washed with brine, dried over anhydrous Na₂SO₄, filtered and concentrated in vacuum to give a residue which was purified by column chromatography to afford title compound (0.38 g). ¹H NMR (400 MHz, CDCl₃) δ 3.96 (s, 1H), 3.50 (t, J=6.4 Hz, 2H), 3.28 (td, J=6.4, 5.6 Hz, 2H), 2.16 (p, J=6.4 Hz, 2H).

2. Synthesis of compound N-(3-bromopropyl)-N-methylcyanamide. To a solution of N-(3-bromopropyl)cyanamide (0.38 g, 2.33 mmol) in anhydrous DMF (10 mL) was added iodomethane (397 mg, 2.80 mmol, 0.17 mL) and K₂CO₃ (967 mg, 6.99 mmol). The mixture was stirred at 25° C. for 12 h. The reaction mixture was quenched by sat. NH₄Cl and extracted with EA. The combined organic phase was washed with brine, dried over anhydrous Na₂SO₄, filtered and concentrated. The residue was purified by column chromatography to afford title compound (0.26 g).

3. Synthesis of compound 5,5-dimethyl-2-(((1-methyl-1,4,5,6-tetrahydropyrimidin-2-yl)oxy)methyl)-7-(4-phenoxyphenyl)-6,7-dihydro-5H-pyrrolo[2,3-d]pyrimidine. To a mixture of (5,5-dimethyl-7-(4-phenoxyphenyl)-6,7-dihydro-5H-pyrrolo[2,3-d]pyrimidin-2-yl)methanol (0.2 g, 0.58 mmol) and N-(3-bromopropyl)-N-methylcyanamide (204 mg, 1.15 mmol) in anhydrous 1,4-dioxane (10 mL) was added dichlorozinc (235 mg, 1.73 mmol) at 25° C. The mixture was stirred at 90° C. for 12 h. The mixture was quenched by aq. NaHCO₃ and extracted with EA. The combined organic phase was washed with brine, dried over Na₂SO₄, filtered and concentrated in vacuum to give a residue. The residue was dissolved in THF (6 mL) and saturated aq. NaHCO₃ (3 mL). The mixture was stirred at 25° C. for 2 h. The mixture was diluted with EA, dried over Na₂SO₄, filtered and concentrated in vacuum to give a residue. The residue was purified by prep-HPLC to afford title compound (6 mg). LCMS (ESI): m/z, 444.2 [M+H]⁺.

### Example 216. Preparation of Compound E152

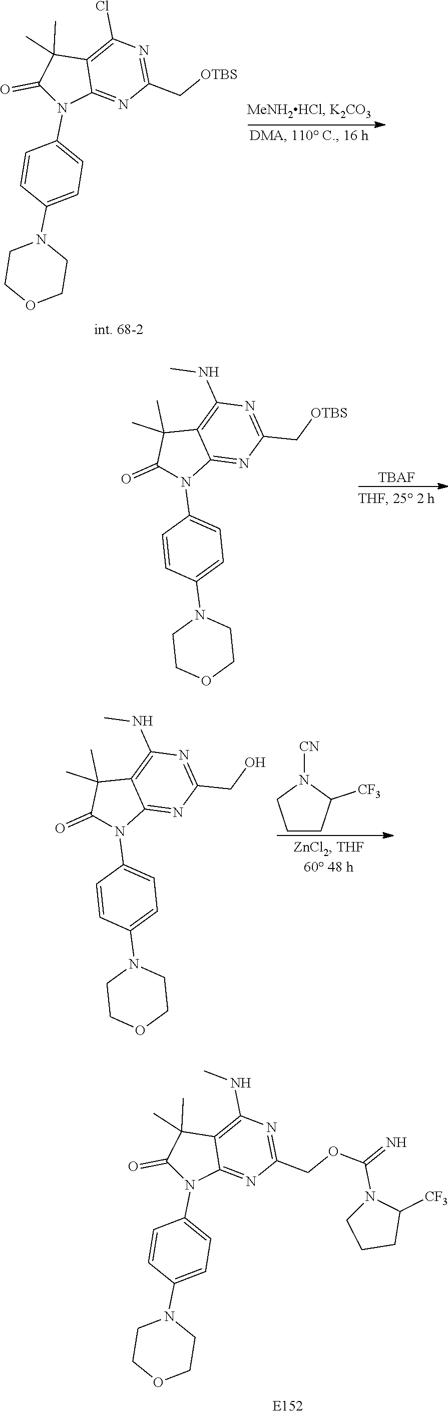

1. Synthesis of compound 2-[[tert-butyl(dimethyl)silyl]oxymethyl]-5,5-dimethyl-4-(methylamino)-7-(4-morpholinophenyl)pyrrolo[2,3-d]pyrimidin-6-one. To a solution of 2-[[tert-butyl(dimethyl)silyl]oxymethyl]-4-chloro-5,5-dimethyl-7-(4-morpholinophenyl)pyrrolo[2,3-d]pyrimidin-6-one (1.0 g, 1.99 mmol) in DMA (15 mL) were added MeNH₂·HCl (403 mg, 5.96 mmol) and K₂CO₃ (1.10 g, 7.95 mmol). The mixture was stirred at 110° C. for 16 h. The mixture was filtered and the filtrate was treated with water, extracted with EtOAc. The combined organic phases were washed with water, brine, dried over anhydrous Na₂SO₄, filtered and concentrated. The residue was purified by column chromatography to afford title compound (700 mg). LCMS (ESI): m/z, 498.3 [M+H]⁺.

2. Synthesis of compound 2-(hydroxymethyl)-5,5-dimethyl-4-(methylamino)-7-(4-morpholinophenyl)pyrrolo[2,3-d]pyrimidin-6-one. To a solution of 2-[[tert-butyl(dimethyl)silyl]oxymethyl]-5,5-dimethyl-4-(methylamino)-7-(4-morpholinophenyl)pyrrolo[2,3-d]pyrimidin-6-one (700 mg, 1.41 mmol) in THF (10 mL) was added TBAF (4.22 mL, 1 M). The mixture was stirred at 25° C. for 2 h. Water was added and extracted with EtOAc. The combined organic phases were washed with water, brine, dried over anhydrous Na₂SO₄, filtered and concentrated. The residue was purified by column chromatography to afford title compound (300 mg). ¹H NMR (400 MHz, CDCl₃) δ 7.38-7.31 (m, 2H), 7.01-6.94 (m, 2H), 4.55 (s, 2H), 4.46 (q, J=4.8 Hz, 1H), 3.92-3.82 (m, 4H), 3.78 (s, 1H), 3.25-3.17 (m, 4H), 3.12 (d, J=4.8 Hz, 3H), 1.51 (s, 6H). LCMS (ESI): m/z, 384.2 [M+H]⁺.

3. Synthesis of compound [5,5-dimethyl-4-(methylamino)-7-(4-morpholinophenyl)-6-oxo-pyrrolo[2,3-d]pyrimidin-2 yl]methyl 2-(trifluoromethyl)pyrrolidine-1-carboximidate. To a solution of 2-(hydroxymethyl)-5,5-dimethyl-4-(methylamino)-7-(4-morpholinophenyl)pyrrolo[2,3-d]pyrimidin-6-one (50 mg, 130 μmol) and 2-(trifluoromethyl)pyrrolidine-1-carbonitrile (64.2 mg, 0.39 mmol) in THF (4 mL) was added ZnCl₂ (71.1 mg, 0.52 mmol). The mixture was stirred at 60° C. for 48 h under N₂. The mixture was filtered. The filtrate was purified by prep-HPLC to afford title compound (10.5 mg). ¹H NMR (400 MHz, CDCl₃) δ 7.40-7.31 (m, 2H), 7.00-6.91 (m, 2H), 5.27-4.96 (m, 2H), 4.48-4.46 (m, 1H), 4.37 (q, J=4.8 Hz, 1H), 3.92-3.80 (m, 4H), 3.53-3.36 (m, 2H), 3.22-3.14 (m, 4H), 3.08 (d, J=4.8 Hz, 3H), 2.05 (q, J 8.0 Hz, 2H), 1.91-1.78 (m, 2H), 1.48 (s, 6H). LCMS (ESI): m/z, 548.3 [M+H]⁺.

### Example 217. Preparation of Compound E153

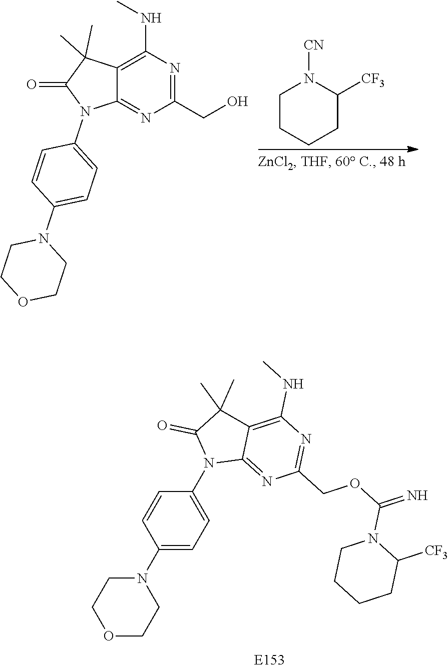 [SMILES: CNc1nc(CO)nc2c1C(C)(C)C(=O)N2c1ccc(N2CCOCC2)cc1 ; N#CN1CCCCC1C(F)(F)F ; CNc1nc(COC(=N)N2CCCCC2C(F)(F)F)nc2c1C(C)(C)C(=O)N2c1ccc(N2CCOCC2)cc1]

To a solution of 2-(hydroxymethyl)-5,5-dimethyl-4-(methylamino)-7-(4-morpholinophenyl)pyrrolo[2,3-d]pyrimidin-6-one (50 mg, 0.13 mmol) and 2-(trifluoromethyl)piperidine-1-carbonitrile (69.7 mg, 0.39 mmol) in THF (4 mL) was added ZnCl₂ (71 mg, 0.52 mmol). The mixture was stirred at 60° C. for 48 h under N₂. The mixture was filtered. The filtrate was purified by prep-HPLC to afford title compound (6.1 mg). ¹H NMR (400 MHz, CDCl₃) δ 7.39-7.32 (m, 2H), 7.00-6.92 (m, 2H), 5.13-4.92 (m, 2H), 4.86 (dd, J=9.6, 5.6 Hz, 1H), 4.40 (q, J=4.8 Hz, 1H), 4.00 (d, J=13.2 Hz, 1H), 3.93-3.75 (m, 4H), 3.24-3.13 (m, 4H), 3.09 (d, J=4.8 Hz, 3H), 3.06-2.96 (m, 1H), 1.97-1.88 (m, 1H), 1.73-1.62 (m, 4H), 1.49 (s, 6H), 1.35-1.22 (m, 1H). LCMS (ESI): m/z, 562.3 [M+H]⁺.

### Example 218. Preparation of Compound E154

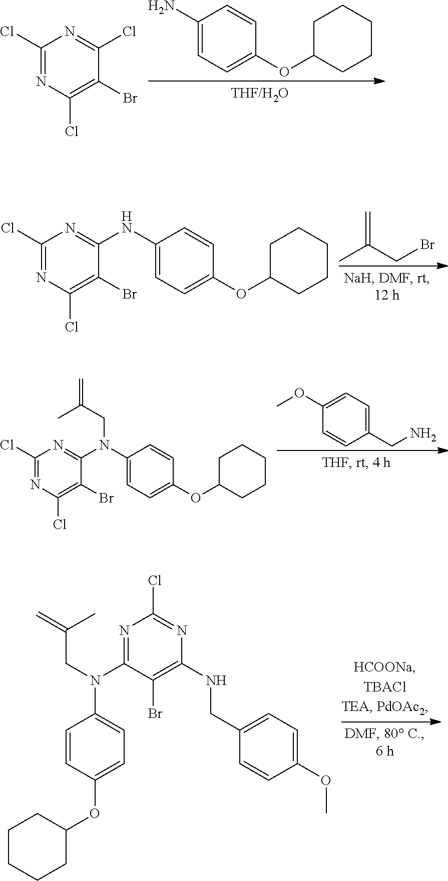

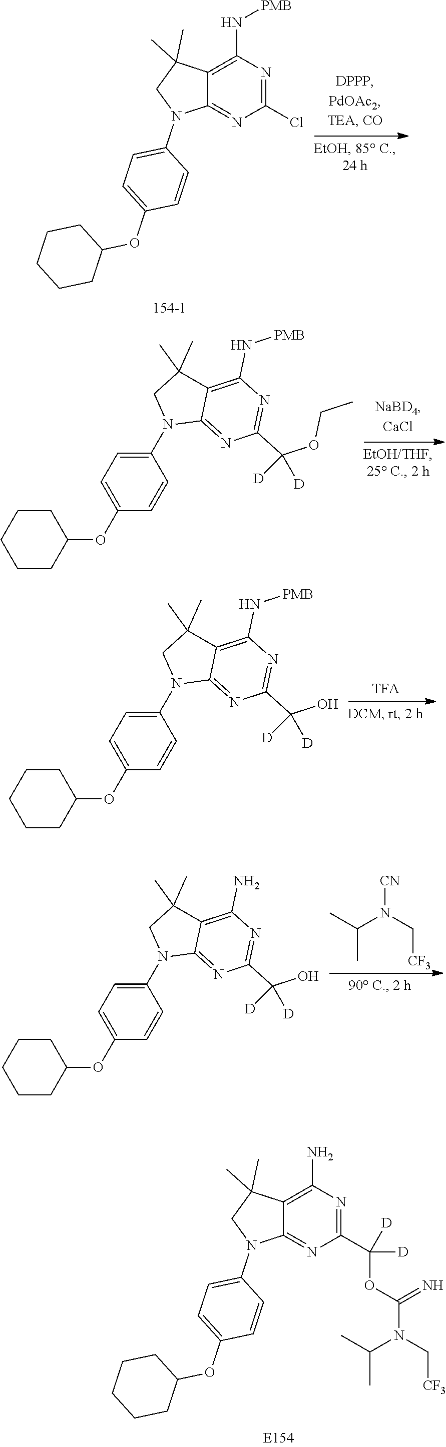

1. Synthesis of compound 154-1. Compound 154-1 was synthesized as compound int. 11-4. LCMS (ESI): m/z, 493.2, 495.2 [M+H]⁺. ¹H NMR (400 MHz, CDCl₃) δ 7.52-7.46 (m, 2H), 7.27 (s, 1H), 7.25 (s, 1H), 6.90 (m, 4H), 4.63 (s, 2H), 4.39 (t, J=5.6 Hz, 1H), 4.18 (m, 1H), 3.81 (s, 3H), 3.69 (s, 2H), 2.02-1.94 (m, 2H), 1.80 (m, 2H), 1.51 (m, 4H), 1.37 (s, 6H), 1.35-1.29 (m, 2H).

2. Synthesis of compound ethyl 7-(4-(cyclohexyloxy)phenyl)-4-((4-methoxybenzyl)amino)-5,5-dimethyl-6,7-dihydro-5H-pyrrolo[2,3-d]pyrimidine-2-carboxylate. To a solution of 2-chloro-7-(4-(cyclohexyloxy)phenyl)-N-(4-methoxybenzyl)-5,5-dimethyl-6,7-dihydro-5H-pyrrolo[2,3-d]pyrimidin-4-amine (1.4 g, 2.84 mmol) in EtOH (20 mL) were added 3-diphenylphosphanylpropyl(diphenyl)phosphane (234 mg, 0.57 mmol), TEA (0.99 mL) and Pd (OAc)₂ (128 mg, 0.57 mmol). The mixture was stirred at 85° C. for 24 h. The reaction mixture was concentrated and the residue was purified by column chromatography to afford title compound (1.1 g). LCMS (ESI): m/z, 531.5 [M+H]⁺. ¹H NMR (400 MHz, CDCl₃) δ: 7.66-7.59 (m, 2H), 7.33-7.28 (m, 2H), 6.93-6.87 (m, 4H), 4.72 (m, 2H), 4.44-4.40 (m, 2H), 4.17 (m, 1H), 3.81 (s, 3H), 3.70 (s, 2H), 2.00-1.94 (m, 2H), 1.79 (m, 2H), 1.56-1.45 (m, 4H), 1.43 (t, J=7.2 Hz, 3H), 1.38 (s, 6H), 1.32 (m, 2H).

3. Synthesis of compound (7-(4-(cyclohexyloxy)phenyl)-4-((4-methoxybenzyl)amino)-5,5-dimethyl-6,7-dihydro-SH-pyrrolo[2,3-d]pyrimidin-2-yl)methan-d₂-ol. To a mixture of 7-(4-(cyclohexyloxy)phenyl)-4-((4-methoxybenzyl)amino)-5,5-dimethyl-6,7-dihydro-5H-pyrrolo[2,3-d]pyrimidine-2-carboxylate (1.1 g, 2.07 mmol) in EtOH (1 mL) was added sodium tetradeuterioboranuide (174 mg, 4.15 mmol) and CaCl₂ (460 mg, 4.15 mmol) at 0° C. The mixture was stirred at 25% C for 3 h. The reaction mixture was quenched by H₂O and extracted with EA. The combined organic phase was washed with brine, dried over anhydrous Na₂SO₄, filtered and concentrated under reduced pressure to give the residue. The residue was purified by column chromatography to afford title compound (0.9 g). LCMS (ESI): m/z, 491.5 [M+H]⁺.

4. Synthesis of compound (4-amino-7-(4-(cyclohexyloxy)phenyl)-5,5-dimethyl-6,7-dihydro-5H-pyrrolo[2,3-d]pyrimidin-2-yl)methan-d₂-ol. A solution of (7-(4-(cyclohexyloxy)phenyl)-4-((4-methoxybenzyl)amino)-5,5-dimethyl-6,7-dihydro-5H-pyrrolo[2,3-d]pyrimidin-2-yl)methan-d₂-ol (0.9 g, 1.83 mmol) in DCM (10 mL) and TFA (10 mL) was stirred at 25° C. for 2 h. The reaction was concentrated and the residue was purified by column chromatography to afford title compound (0.5 g). LCMS (ESI): m/z, 371.3 [M+H]⁺.

5. Synthesis of compound ethyl (4-amino-7-(4-(cyclohexyloxy)phenyl)-5,5-dimethyl-6,7-dihydro-5H-pyrrolo[2,3-d]pyrimidin-2-yl)methyl-d₂ isopropyl(2,2,2-trifluoroethyl)carbamimidate. To a solution of (4-amino-7-(4-(cyclohexyloxy)phenyl)-5,5-dimethyl-6,7-dihydro-5H-pyrrolo[2,3-d]pyrimidin-2-yl)methan-d₂-ol (0.15 g, 0.40 mmol) and isopropyl(2,2,2-trifluoroethyl)cyanamide (202 mg, 1.21 mmol) in anhydrous 1,4-dioxane (5 mL) was added dichlorozinc (1 M, 1.62 mL) at 25° C. The mixture was stirred at 90° C. for 2 h. The reaction mixture was concentrated and the residue was purified by prep-HPLC to afford title compound (55 mg). LCMS (ESI): m/z, 537.3 [M+H]. ¹H NMR (400 MHz, DMSO-d₆) δ: 7.65-7.59 (m, 2H), 6.86 (d, J=9.2 Hz, 2H), 6.12 (s, 2H), 4.22 (m, 2H), 4.08 (q, J=9.6 Hz, 2H), 3.64 (s, 2H), 1.90 (d, J=9.6 Hz, 2H), 1.74-1.66 (m, 2H), 1.56-1.41 (m, 4H), 1.36 (s, 6H), 1.34-1.25 (m, 2H), 1.10 (d, J=6.8 Hz, 6H).

### Example 219. Preparation of Compound E155

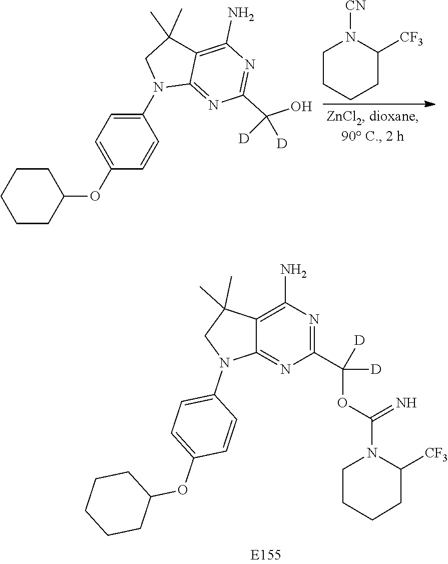

To a solution of 2-(trifluoromethyl)piperidine-1-carbonitrile (72 mg, 0.40 mmol) and (4-amino-7-(4-(cyclohexyloxy)phenyl)-5,5-dimethyl-6,7-dihydro-5H-pyrrolo[2,3-d]pyrimidin-2-yl)methan-d₂-ol (50 mg, 0.13 mmol) in anhydrous dioxane (2 mL) was added ZnCl₂ (74 mg, 0.54 mmol) at 25° C. under N₂. The mixture was stirred at 90° C. for 4 h. The reaction mixture was filtered and concentrated under reduced pressure. The residue was purified by prep-HPLC to afford title compound (30 mg). LCMS (ESI): m/z, 549.3 [M+H]⁺. ¹H NMR (400 MHz, CDCl₃) δ 7.57 (d, J=8.4 Hz, 2H), 6.90 (d, J=8.4 Hz, 2H), 4.90 (d, J=10.4 Hz, 1H), 4.58 (s, 2H), 4.26-3.95 (m, 2H), 3.71 (s, 2H), 3.07 (t, J=13.2 Hz, 1H), 1.99 (t, J=9.2 Hz, 3H), 1.85-1.63 (m, 7H), 1.59-1.27 (m, 13H).

### Example 220. Preparation of Compound E156

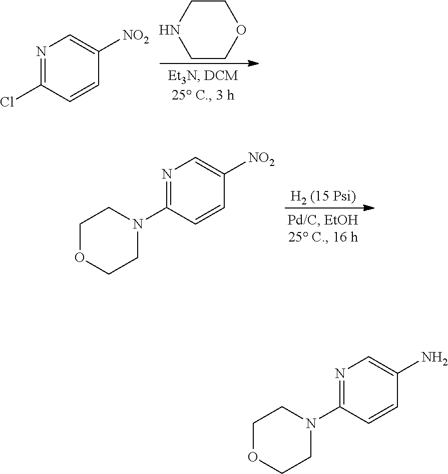 [SMILES: O=[N+]([O-])c1ccc(Cl)nc1 ; C1COCCN1 ; O=[N+]([O-])c1ccc(N2CCOCC2)nc1 ; "H2 (15 Psi)" ; Nc1ccc(N2CCOCC2)nc1]

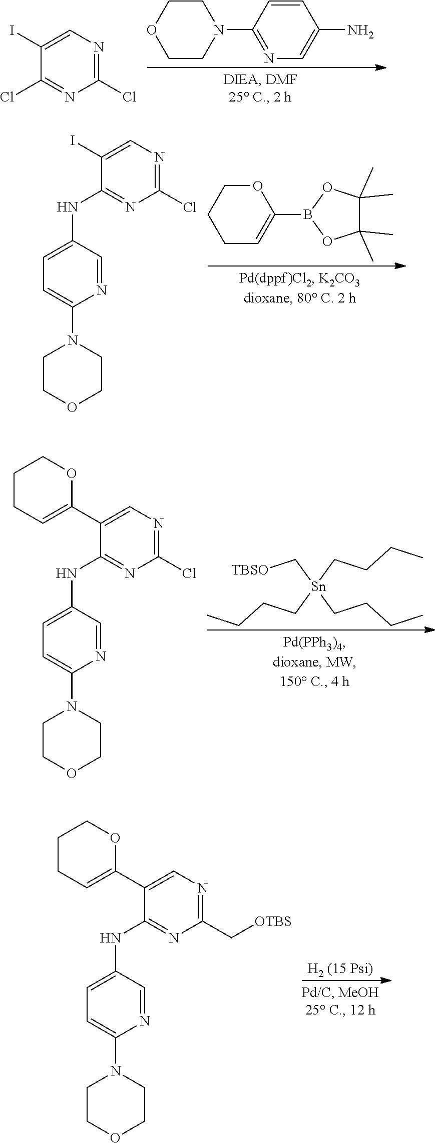

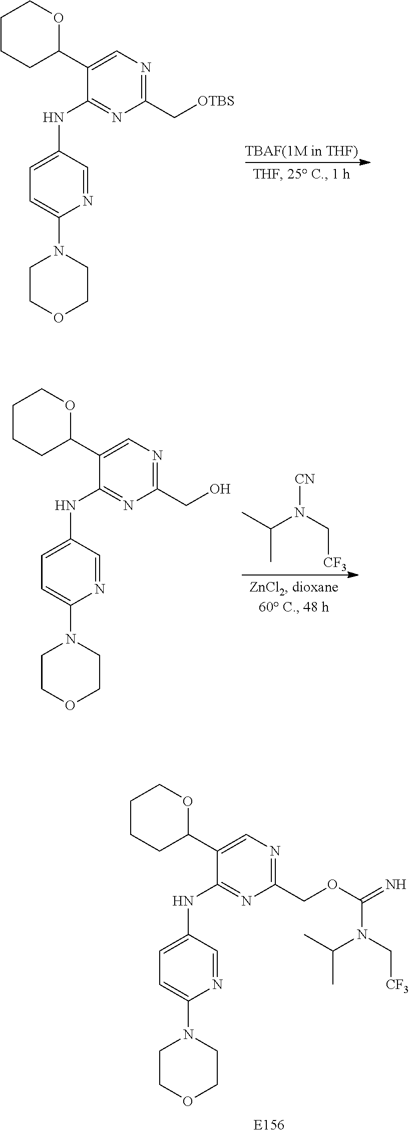

1. Synthesis of compound 4-(5-nitropyridin-2-yl)morpholine. To a solution of 2-chloro-5-nitro-pyridine (5.0 g, 31.5 mmol), morpholine (13 mL) in DCM (30 mL) was added Et₃N (10.1 mL). The mixture was stirred at 25° C. for 3 hours. The mixture was concentrated. The residue was purified by column chromatography to afford title compound (6.2 g). ¹H NMR (400 MHz, DMSO-d₆) δ 8.97 (d, J=2.8 Hz, 1H), 8.24 (dd, J=2.8, 9.2 Hz, 1H), 8.94 (d, J=9.6 Hz, 1H), 3.74-3.72 (m, 4H), 3.70-3.68 (m, 4H). LCMS (ESI): m/z, 210.1 [M+H]⁺.

2. Synthesis of compound 6-morpholinopyridin-3-amine. To a solution of 4-(5-nitropyridin-2-yl)morpholine (6.2 g, 29.6 mmol) in EtOH (100 mL) was added Pd/C (6.31 g, 5.93 mmol, 10% purity). The mixture was stirred at 25° C. for 16 hours under H₂ atmosphere. The mixture was filtered. The filtrate was concentrated to afford title compound (4.8 g). ¹H NMR (400 MHz, DMSO-d₆) δ 7.61 (d, J=2.8 Hz, 1H), 6.92 (dd, J=2.8, 8.8 Hz, 1H), 6.62 (d, J=8.8 Hz, 1H), 4.58 (s, 2H), 3.68 (t, J=4.8 Hz, 4H), 3.17 (t, J=4.8 Hz, 4H). LCMS (ESI): m/z, 180.1 [M+H]⁺.

3. Synthesis of compound 2-chloro-5-iodo-N-(6-morpholinopyridin-3-yl)pyrimidin-4-amine. To a solution of 2,4-dichloro-5-iodopyrimidine (3.0 g, 10.9 mmol), 6-morpholinopyridin-3-amine (1.96 g, 10.9 mmol) in DMF (50 mL) was added DIEA (2.71 mL). The mixture was stirred at 25° C. for 2 hours. The mixture was diluted with EA, washed with sat. NaCl, dried over Na₂SO₄, filtered and concentrated. The residue was purified by column chromatography to afford title compound (3.8 g). LCMS (ESI): m/z, 418.0, 420.0 [M+H]⁺.

4. Synthesis of compound 2-chloro-5-(3,4-dihydro-2H-pyran-6-yl)-N-(6-morpholinopyridin-3-yl)pyrimidin-4-amine. A mixture of 2-chloro-5-iodo-N-(6-morpholinopyridin-3-yl)pyrimidin-4-amine (1.5 g, 3.59 mmol), 2-(3,4-dihydro-2H-pyran-6-yl)-4,4,5,5-tetramethyl-1,3,2-dioxaborolane (830 mg, 3.95 mmol), Pd(dppf)Cl₂ (263 mg, 0.36 mmol), K₂CO₃ (993 mg, 7.18 mmol) in dioxane (20 mL) and H₂O (4 mL) was stirred at 80° C. for 2 hours. The mixture was concentrated. The residue was purified by column chromatography to afford title compound (1.1 g). ¹H NMR (400 MHz, DMSO-d₆) b 8.66 (s, 1H), 8.19 (d, J=2.4 Hz, 1H), 8.08 (s, 1H), 7.64 (dd, J=2.8, 9.2 Hz, 1H), 6.87 (d, J=5.6 Hz, 1H), 5.19 (t, J=4.0 Hz, 1H), 4.17 (t, J=4.8 Hz, 2H), 3.71 (t, J=4.8 Hz, 4H), 3.42 (t, J=4.8 Hz, 4H), 2.16-2.12 (m, 2H), 1.88-1.82 (m, 2H). LCMS (ESI): m/z, 374.1, 376.1 [M+H]⁺.

5. Synthesis of compound 2-(((tert-butyldimethylsilyl)oxy)methyl)-5-(3,4-dihydro-2H-pyran-6-yl)-N-(6-morpholinopyridin-3-yl)pyrimidin-4-amine. A mixture of 2-chloro-5-(3,4-dihydro-2H-pyran-6-yl)-N-(6-morpholinopyridin-3-yl)pyrimidin-4-amine (500 mg, 1.34 mmol), tert-butyl-dimethyl-(tributylstannylmethoxy)silane (756 mg, 1.74 mmol), Pd(PPh₃)₄ (231 mg, 0.20 mmol) in dioxane (15 mL) was stirred at 130° C. for 4 hours under N₂ atmosphere by microwave. The mixture was concentrated. The residue was purified by column chromatography to afford title compound (230 mg). LCMS (ESI): m/z, 484.3 [M+H]⁺.

6. Synthesis of compound 2-(((tert-butyldimethylsilyl)oxy)methyl)-N-(6-morpholinopyridin-3-yl)-5-(tetrahydro-2H-pyran-2-yl)pyrimidin-4-amine. To a solution of 2-chloro-5-(3,4-dihydro-2H-pyran-6-yl)-N-(6-morpholinopyridin-3-yl)pyrimidin-4-amine (230 mg, 0.47 mmol) in MeOH (14 mL) was added Pd/C (10 wt %, 152 mg, 1.43 mmol). The mixture was stirred at 25° C. for 12 hours under H₂ atmosphere. The mixture was filtered. The filtrate was concentrated to afford title compound (230 mg). LCMS (ESI): m/z, 486.3 [M+H]⁺. [001228]7. Synthesis of compound (4-((6-morpholinopyridin-3-yl)amino)-5-(tetrahydro-2H-pyran-2-yl)pyrimidin-2-yl)methanol. To a solution of 2-[[tert-butyl(dimethyl)silyl]oxymethyl]-N-(6-morpholino-3-pyridyl)-5-tetrahydropyran-2-yl-pyrimidin-4-amine (180 mg, 0.37 mmol) in THF (3 mL) was added TBAF (1 M, 1.11 mL). The mixture was stirred at 25° C. for 1 hour. The mixture was concentrated. The residue was purified by column chromatography to afford title compound (110 mg). ¹H NMR (400 MHz, DMSO-d₆) δ 8.40 (s, 1H), 8.38 (d, J=2.8 Hz, 1H), 8.20 (s, 1H), 7.83 (dd, J=2.8, 8.4 Hz, 1H), 6.84 (d, J=9.2 Hz, 1H), 4.83 (t, J=6.0 Hz, 1H), 4.33 (d, J=6.0 Hz, 2H), 3.71 (t, J=4.8 Hz, 4H), 3.40 (t, J=4.8 Hz, 4H), 3.18-3.14 (m, 2H), 1.89-1.82 (m, 2H), 1.72-1.53 (m, 4H). LCMS (ESI): m/z, 372.2 [M+H]⁺.

8. Synthesis of compound (4-((6-morpholinopyridin-3-yl)amino)-5-(tetrahydro-2H-pyran-2-yl)pyrimidin-2-yl)methyl isopropyl. To a solution of (4-((6-morpholinopyridin-3-yl)amino)-5-(tetrahydro-2H-pyran-2-yl)pyrimidin-2-yl)methanol (110 mg, 0.29 mmol), isopropyl(2,2,2-trifluoroethyl)cyanamide (148 mg, 0.88 mmol) in dioxane (10 mL) was added ZnCl₂ (162 mg, 1.18 mmol). The mixture was stirred at 60° C. for 48 hours under N₂ atmosphere. The mixture was filtered and the filtrate was concentrated. The residue was purified by pre-HPLC to afford title compound (40 mg). ¹H NMR (400 MHz, DMSO-d₆) δ 8.41 (s, 1H), 8.29 (d, J=2.8 Hz, 1H), 8.18 (s, 1H), 7.01 (dd, J=2.8, 8.8 Hz, 1H), 6.82 (d, J=9.2 Hz, 1H), 6.21 (s, 1H), 4.96 (s, 2H), 4.59-4.56 (m, 1H), 4.07-4.00 (m, 2H), 3.98-3.91 (m, 2H), 3.71 (t, J=4.8 Hz, 4H), 3.65-3.59 (m, 1H), 3.40 (t, J=4.8 Hz, 4H), 1.89-1.82 (m, 2H), 1.72-1.54 (m, 4H), 1.04 (d, J=6.8 Hz, 6H). LCMS (ESI): m/z, 538.3 [M+H]⁺.

### Example 221. Preparation of Compound E157

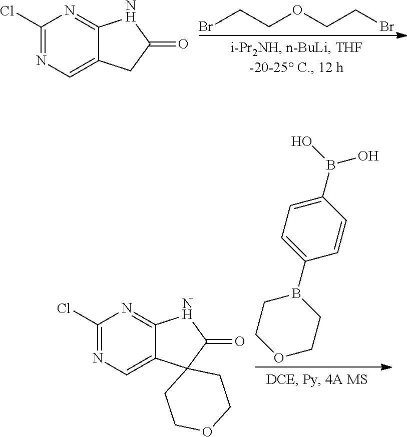

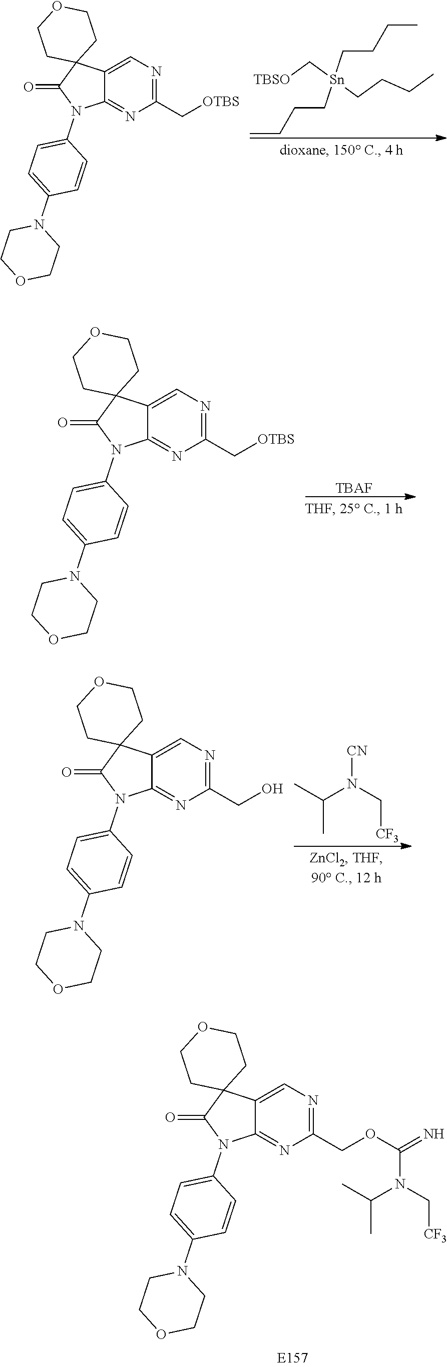

1. Synthesis of compound 2′-chloro-2,3,5,6-tetrahydrospiro[pyran-4,5′-pyrrolo[2,3-d]pyrimidin]-6′(7′H)-one. To a stirred solution of 2-chloro-5,7-dihydro-6H-pyrrolo[2,3-d]pyrimidin-6-one (10.0 g, 59.0 mmol) in dry THF (200 mL) was added i-Pr₂NH (17.90 g, 177 mmol, 25.0 mL) followed by n-BuLi (2.5 M, 94.4 mL) at −20° C. The reaction mixture was stirred at −20° C. for 0.5 h and allowed to warm to 0° C. and then 1-bromo-2-(2-bromoethoxy)ethane (41.0 g, 177 mmol) was added. The reaction mixture was stirred at 25° C. for 12 h. The reaction was quenched with sat. NH₄Cl, brine and extracted with EtOAc. The combined organic layer was washed with brine, dried over anhydrous Na₂SO₄, filtered and concentrated. The residue was purified by column chromatography to afford title compound (0.6 g). LCMS (ESI): m/z, 240.1, 242.0 [M+H]⁺.

2. Synthesis of compound 2′-chloro-7′-(4-morpholinophenyl)-2,3,5,6-tetrahydrospiro[pyran-4,5′-pyrrolo[2,3-d]pyrimidin]-6′(7′H)-one. To a solution of 2′-chloro-2,3,5,6-tetrahydrospiro[pyran-4,5′-pyrrolo[2,3-d]pyrimidin]-6′(7′H)-one (0.3 g, 1.25 mmol) and (4-morpholinophenyl)boronic acid (777 mg, 3.76 mmol) in DCE (10 mL) was added Cu(OAc)₂ (272 mg, 1.50 mmol), Py (0.1 mL) and 4 Å molecular sieve (300 mg) under O₂. The reaction was stirred at 25° C. for 12 h. The mixture was filtered by diatomite and washed with EA. The organic phase was concentrated and purified by column chromatography to afford title compound (0.4 g). LCMS (ESI): m/z, 401.2, 403.2 [M+H]⁺.

3. Synthesis of compound 2′-(((tert-butyldimethylsilyl)oxy)methyl)-7′-(4-morpholinophenyl)-2,3,5,6-tetrahydrospiro[pyran-4,5′-pyrrolo[2,3-d]pyrimidin]-6′(7′H)-one. To a mixture of 2′-chloro-7′-(4-morpholinophenyl)-2,3,5,6-tetrahydrospiro[pyran-4,5′-pyrrolo[2,3-d]pyrimidin]-6′(7′H)-one (0.3 g, 0.75 mmol) in dioxane (10 mL) was added tert-butyl-dimethyl-(tributylstannylmethoxy)silane (423 mg, 0.97 mmol) and Pd(PPh₃)₄ (173 mg, 0.15 mmol). The mixture was purged by N₂ and stirred at 150° C. for 4 h under microwave. The reaction mixture was concentrated under reduced pressure to give a residue which was purified by column chromatography to afford title compound (0.2 g). LCMS (ESI): m/z, 511.3 [M+H]⁺.

4. Synthesis of compound 2′-(hydroxymethyl)-7′-(4-morpholinophenyl)-2,3,5,6-tetrahydrospiro[pyran-4,5′-pyrrolo[2,3-d]pyrimidin]-6′(7′H)-one. To a solution of 2′-(((tert-butyldimethylsilyl)oxy)methyl)-7′-(4-morpholinophenyl)-2,3,5,6-tetrahydrospiro[pyran-4,5′-pyrrolo[2,3-d]pyrimidin]-6′(7′H)-one (0.2 g, 0.39 mmol) in THE (5 mL) was added TBAF (1 M, 1.17 mL) at 25° C. The mixture was stirred at 25° C. for 1 h. The reaction mixture was concentrated and purified by prep-TLC to afford title compound (52 mg). LCMS (ESI): m/z, 397.1 [M+H]⁺.

5. Synthesis of compound (7′-(4-morpholinophenyl)-6′-oxo-2,3,5,6,6′,7′-hexahydrospiro[pyran-4,5′-pyrrolo[2,3-d]pyrimidin]-2′-yl)methyl isopropyl(2,2,2-trifluoroethyl)carbamimidate. A mixture of isopropyl(2,2,2-trifluoroethyl)cyanamide (63 mg, 0.38 mmol), 2-(hydroxymethyl)-7-(4-morpholinophenyl)spiro[pyrrolo[2,3-d]pyrimidine-5,4′-tetrahydropyran]-6-one (50 mg, 0.13 mmol) and ZnCl₂ (69 mg, 0.50 mmol) in dioxane (4.5 mL) was stirred at 90° C. for 12 h under N₂. The reaction mixture was concentrated under reduced pressure to give a residue which was purified by prep-HPLC to afford title compound (17.3 mg). ¹H NMR (400 MHz, CDCl₃) δ 8.62 (s, 1H), 7.34-7.31 (m, 2H), 7.00-6.95 (m, 2H), 5.24 (s, 2H), 4.24-4.18 (m, 2H), 4.10-4.03 (m, 1H), 3.93-3.86 (m, 6H), 3.83-3.76 (m, 2H), 3.21-3.19 (m, 4H), 2.18-2.12 (m, 2H), 1.86-1.81 (m, 2H), 1.16 (s, 3H), 1.15 (s, 3H). LCMS (ESI): m/z, 563.3 [M+H]⁺.

### Example 222. Preparation of Compound E158

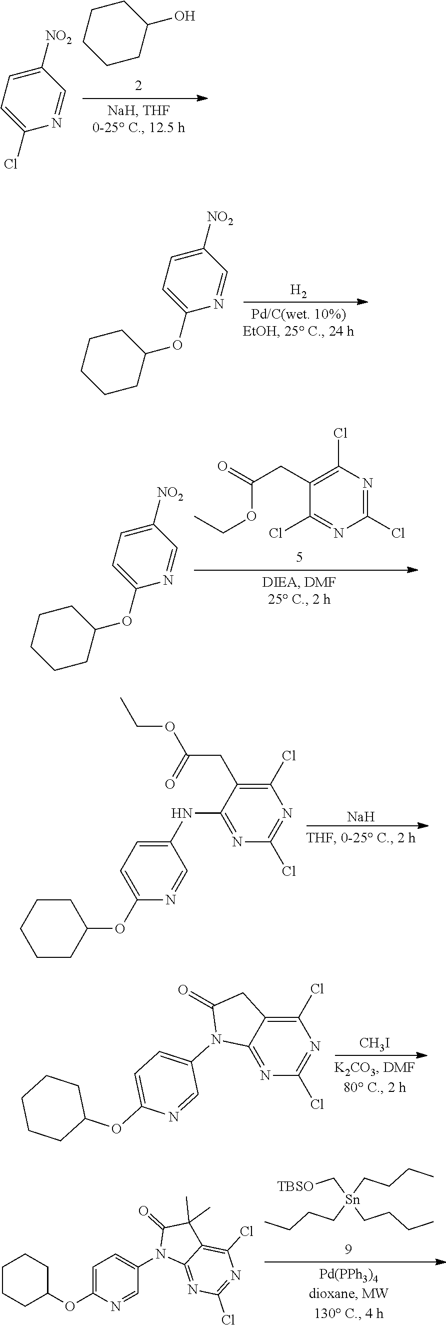

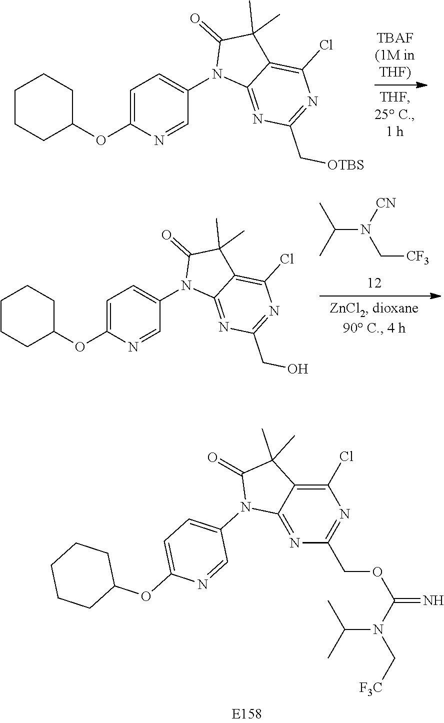

1. Synthesis of compound 22-(cyclohexyloxy)-5-nitropyridine. To a solution of cyclohexanol (20.8 g, 208 mmol) in THF (300 mL) was added NaH (11.3 g, 284 mmol, 60% purity) at 0° C. The mixture was stirred at 0° C. for 0.5 h, then 2-chloro-5-nitro-pyridine (30.0 g, 189 mmol) was added. The mixture was stirred at 25° C. for 12 hours. The mixture was quenched by sat. NH₄Cl, extracted with EA, then combined organic layers, dried over Na₂SO₄, filtered and concentrated. The residue was purified by column chromatography to afford title compound (31.0 g). ¹H NMR (400 MHz, CDCl₃) 4 9.05 (d, J=2.8 Hz, 1H), 8.32 (dd, J=2.8, 9.2 Hz, 1H), 6.75 (d, J=9.2 Hz, 1H), 5.21-5.14 (m, 1H), 2.04-1.99 (m, 2H), 1.84-1.76 (m, 2H), 1.55-1.28 (m, 6H).

2. Synthesis of compound 6-(cyclohexyloxy)pyridin-3-amine. To a solution of 2-(cyclohexoxy)-5-nitro-pyridine (31 g, 139.49 mmol) in EtOH (100 mL) was addedPd/C (10.4 g, 9.76 mmol, 10% purity). The mixture was stirred at 25° C. for 24 hours under H₂ atmosphere. The mixture was filtered. The filtrate was concentrated to afford title compound (25 g). ¹H NMR (400 MHz, DMSO-d₆) δ 7.47 (d, J=3.2 Hz, ¹H), 6.97 (dd, J=2.8, 8.4 Hz, 1H), 6.47 (d, J=8.4 Hz, ¹H), 4.77-4.71 (m, 3H), 1.91-1.84 (m, 2H), 1.74-1.66 (m, 2H), 1.55-1.21 (m, 6H).

3. Synthesis of compound ethyl 2-(2,4-dichloro-6-((6-(cyclohexyloxy)pyridin-3-yl)amino)pyrimidin-5-yl)acetate. To a solution of ethyl ethyl 2-(2,4,6-trichloropyrimidin-5-yl)acetate (10.0 g, 37.10 mmol), 6-(cyclohexyloxy)pyridin-3-amine (7.13 g, 37.1 mmol) in DMF (150 mL) was added DIEA (7.36 mL). The mixture was stirred at 25° C. for 2 hours. The mixture was diluted with EA, washed with sat. NaCl, dried over Na₂SO₄, filtered and concentrated. The residue was purified by column chromatography to afford title compound (13.0 g). LCMS (ESI): m/z, 425.0, 427.0 [M+H]⁺.

4. Synthesis of compound 2,4-dichloro-7-(6-(cyclohexyloxy)pyridin-3-yl)-5,7-dihydro-6H-pyrrolo[2,3-d]pyrimidin-6-one. To a solution of methyl ethyl 2-(2,4-dichloro-6-((6-(cyclohexyloxy)pyridin-3-yl)amino)pyrimidin-5-yl)acetate (15.0 g, 36.5 mmol) in THF (150 mL) was added NaH (2.19 g, 54.7 mmol, 60% purity) at 0° C. The mixture was stirred at 25° C. for 2 hours. The mixture was quenched by sat. NH₄Cl, extracted with EA, washed with water, dried over Na₂SO₄, filtered and concentrated. The residue was purified by column chromatography to afford title compound (12.0 g). ¹H NMR (400 MHz, CDCl₃) δ 8.23 (d, J=2.8 Hz, 1H), 7.60 (dd, J=2.8, 8.8 Hz, 1H), 6.83 (d, J=8.8 Hz, 1H), 5.12-5.05 (m, 1H), 3.77 (s, 2H), 2.06-2.00 (m, 2H), 1.85-1.78 (m, 2H), 1.55-1.25 (m, 6H). LCMS (ESI): m/z, 379.0, 381.0 [M+H]⁺.

5. Synthesis of compound 2,4-dichloro-7-(6-(cyclohexyloxy)pyridin-3-yl)-5,5-dimethyl-5,7-dihydro-6H-pyrrolo[2,3-d]pyrimidin-6-one. To a solution of 2,4-dichloro-7-(6-(cyclohexyloxy)pyridin-3-yl)-5,7-dihydro-6H-pyrrolo[2,3-d]pyrimidin-6-one (12 g, 31.64 mmol), iodomethane (22.5 g, 158 mmol) in DMF (120 mL) was added K₂CO₃ (10.9 g, 79.1 mmol). The mixture was stirred at 80° C. for 2 hours. The mixture was diluted with EA, washed with water, dried over Na₂SO₄, filtered and concentrated. The residue was purified by column chromatography to afford title compound (3.0 g). ¹H NMR (400 MHz, CDCl₃) δ 8.47 (d, J=2.8 Hz, 1H), 7.62 (dd, J=2.8, 8.8 Hz, 1H), 6.83 (d, J=8.8 Hz, 1H), 5.12-5.05 (m, 1H), 2.06-2.00 (m, 2H), 1.84-1.78 (m, 2H), 1.64 (s, 6H), 1.59-1.26 (m, 6H). LCMS (ESI): m/z, 407.1, 409.1 [M+H]⁺.

6. Synthesis of compound 2-(((tert-butyldimethylsilyl)oxy)methyl)-4-chloro-7-(6-(cyclohexyloxy)pyridin-3-yl)-5,5-dimethyl-5,7-dihydro-6H-pyrrolo[2,3-d]pyrimidin-6-one. To a solution of 2,4-dichloro-7-(6-(cyclohexyloxy)pyridin-3-yl)-5,5-dimethyl-5,7-dihydro-6H-pyrrolo[2,3-d]pyrimidin-6-one (2.0 g, 4.91 mmol), tert-butyl-dimethyl-(tributylstannylmethoxy)silane (2.78 g, 6.38 mmol) in dioxane (10 mL) was added Pd(PPh₃)₄ (851 mg, 0.74 mmol). The mixture was stirred at 130° C. by microwave for 4 h under N₂ atmosphere. The mixture was concentrated. The residue was purified by column chromatography to afford title compound (1.5 g). LCMS (ESI): m/z, 517.3, 519.3 [M+H]⁺.

7. Synthesis of compound 4-chloro-7-(6-(cyclohexyloxy)pyridin-3-yl)-2-(hydroxymethyl)-5,5-dimethyl-5,7-dihydro-6H-pyrrolo[2,3-d]pyrimidin-6-one. To a solution of 2-(((tert-butyldimethylsilyl)oxy)methyl)-4-chloro-7-(6-(cyclohexyloxy)pyridin-3-yl)-5,5-dimethyl-5,7-dihydro-6H-pyrrolo[2,3-d]pyrimidin-6-one (360 mg, 0.42 mmol) in THF (6 mL) was added TBAF (1 M, 2.0 mL). The mixture was stirred at 25° C. for 1 hours. The mixture was concentrated. The residue was purified by column chromatography to afford title compound (140 mg). LCMS (ESI): m/z, 403.2, 405.2 [M+H]⁺.

8. Synthesis of compound (4-chloro-7-(6-(cyclohexyloxy)pyridin-3-yl)-5,5-dimethyl-6-oxo-6,7-dihydro-5H-pyrrolo[2,3-d]pyrimidin-2-yl)methyl isopropyl(2,2,2-trifluoroethyl)carbamimidate. To a solution of 4-chloro-7-(6-(cyclohexyloxy)pyridin-3-yl)-2-(hydroxymethyl)-5,5-dimethyl-5,7-dihydro-6H-pyrrolo[2,3-d]pyrimidin-6-one (120 mg, 0.29 mmol), isopropyl(2,2,2-trifluoroethyl)cyanamide (148 mg, 0.89 mmol) in dioxane (10 mL) was added ZnCl₂ (162 mg, 1.19 mmol). The mixture was stirred at 90° C. for 4 hours under N₂ atmosphere. The mixture was filtered. The filtrate was concentrated and the residue was purified by prep-HPLC to afford title compound (6 mg). ¹H NMR (400 MHz, CDCl₃) δ: 8.23 (d, J=2.4 Hz, 1H), 7.78 (dd, J=2.8, 8.8 Hz, 1H), 6.91 (d, J=8.8 Hz, 1H), 6.08 (s, 1H), 5.17 (s, 2H), 5.06-5.00 (m, 1H), 4.05-3.99 (m, 1H), 3.95-3.87 (m, 2H), 2.02-1.95 (m, 2H), 1.77-1.72 (m, 2H), 1.53 (s, 6H), 1.51-1.23 (m, 6H), 1.01 (d, J=6.8 Hz, 6H). LCMS (ESI): m/z, 407.1, 409.1 [M+H]⁺.

### Example 223. Preparation of Compound E159

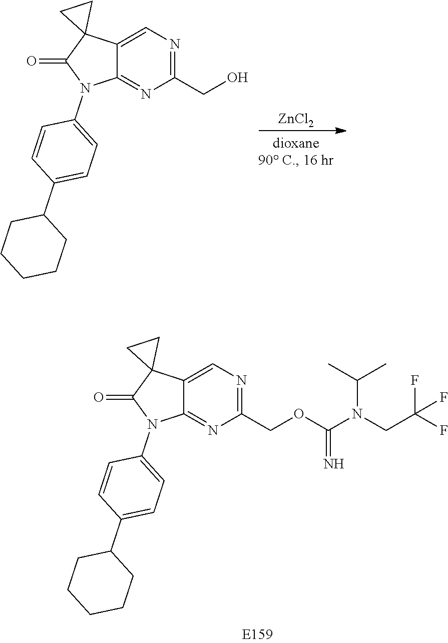

To a solution of 7′-(4-cyclohexylphenyl)-2′-(hydroxymethyl)spiro[cyclopropane-1,5′-pyrrolo[2,3-d]pyrimidin]-6′(7′H)-one (112 mg, 0.32 mmol) and N-isopropyl-N-(2,2,2-trifluoroethyl)cyanamide (160 mg, 0.96 mmol) in Dioxane (2 mL) was added ZnCl₂ (175 mg, 1.28 mmol) and stirred at 90° C. for 16 hours. The reaction was filtered and purified by Prep-HPLC to afford title compound (25 mg). ¹H NMR (400 MHz, CDCl₃) δ 8.06 (s, 1H), 7.41 (d, J=8.4 Hz, 2H), 7.33 (d, J=8.4 Hz, 2H), 5.25 (s, 2H), 4.11-3.98 (m, 1H), 3.78 (q, J=9.2 Hz, 2H), 2.62-2.52 (m, 1H), 1.98-1.95 (m, 2H), 1.93-1.85 (m, 4H), 1.78-1.75 (m, 2H), 1.55-1.32 (m, 6H), 1.12 (d, J=6.8 Hz, 6H). LCMS (ESI): m/z, 516.3 [M+H]⁺.

### Example 224. Preparation of Compound E160

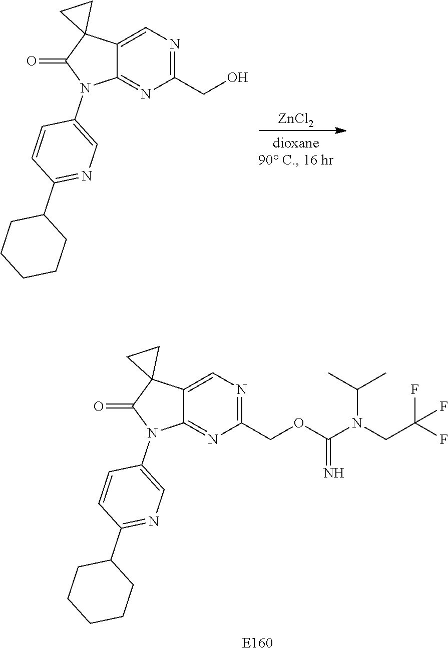

To a solution of 7′-(6-cyclohexylpyridin-3-yl)-2′-(hydroxymethyl)spiro[cyclopropane-1,5′-pyrrolo[2,3-d]pyrimidin]-6′(7′H)-one (118 mg, 0.34 mmol) and N-isopropyl-N-(2,2,2-trifluoroethyl)cyanamide (168 mg, 1.01 mmol) in dioxane (3 mL) was added ZnCl₂ (184 mg, 1.35 mmol) and stirred at 90° C. for 16 hours. The reaction was filtered and purified by Prep-HPLC to afford title compound (15 mg). ¹H NMR (400 MHz, CDCl₃) δ 8.34 (d, J=2.8 Hz, 1H), 8.06 (s, 1H), 7.63 (dd, J=9.2, 2.8 Hz, 1H), 6.72 (d, J=9.2 Hz, 1H), 5.25 (s, 2H), 4.71 (s, 1H), 4.12-4.01 (m, 2H), 3.85-3.83 (m, 4H), 3.64-3.54 (m, 4H), 2.02-1.93 (m, 2H), 1.81-1.73 (m, 2H), 1.25 (d, J=6.8 Hz, 9H), 1.17 (d, J=6.8 Hz, 6H). LCMS (ESI): m/z, 517.3 [M+H]⁺.

### Example 225. Preparation of Compound E161

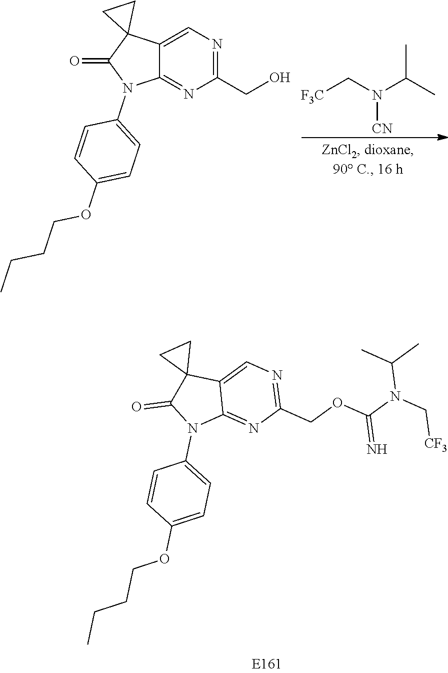 [SMILES: CCCCOc1ccc(N2C(=O)C3(CC3)c3cnc(CO)nc32)cc1 ; CC(C)N(C#N)CC(F)(F)F ; CCCCOc1ccc(N2C(=O)C3(CC3)c3cnc(COC(=N)N(CC(F)(F)F)C(C)C)nc32)cc1]

To a solution of 7′-(4-butoxyphenyl)-2′-(hydroxymethyl)spiro[cyclopropane-1,5′-pyrrolo[2,3-d]pyrimidine]-6′-one (100 mg, 0.29 mmol) and isopropyl(2,2,2-trifluoroethyl)cyanamide (147 mg, 0.88 mmol) in 1,4-dioxane (10 mL) was added ZnCl₂ (161 mg, 1.18 mmol). The mixture was stirred at 90° C. for 16 h under N₂. The mixture was filtered and the filtrate was concentrated. The residue was purified by prep-HPLC to yield title compound (57.1 mg). ¹H NMR (400 MHz, CDCl₃) δ 8.04 (s, 1H), 7.44-7.31 (m, 2H), 7.08-6.89 (m, 2H), 5.22 (s, 2H), 4.13-4.04 (m, 1H), 4.00 (t, J=6.4 Hz, 2H), 3.80 (q, J=9.2 Hz, 2H), 1.95 (q, J=4.4 Hz, 2H), 1.85-1.71 (m, 4H), 1.58-1.43 (m, 2H), 1.14 (d, J=6.8 Hz, 6H), 0.99 (t, J=7.2 Hz, 3H). LCMS (ESI): m/z, 506.2 [M+H]⁺

### Example 226. Preparation of Compound E162

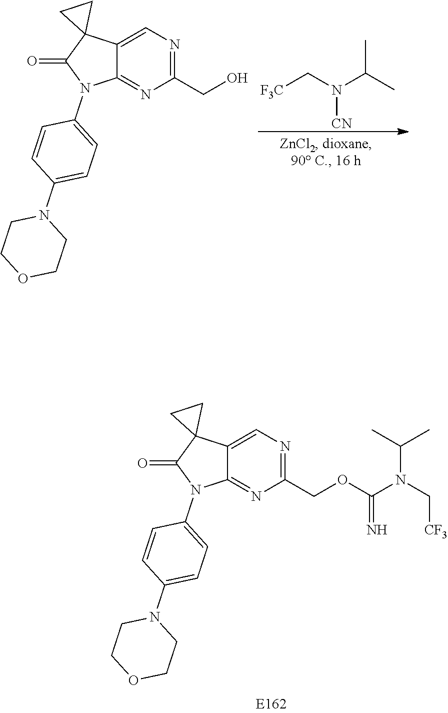 [SMILES: O=C1N(c2ccc(N3CCOCC3)cc2)c2nc(CO)ncc2C12CC2 ; CC(C)N(C#N)CC(F)(F)F ; CC(C)N(CC(F)(F)F)C(=N)OCc1ncc2c(n1)N(c1ccc(N3CCOCC3)cc1)C(=O)C21CC1]

To a solution of 2′-(hydroxymethyl)-7′-(4-morpholinophenyl)spiro [cyclopropane-1,5′-pyrrolo[2,3-d]pyrimidine]-6′-one (159 mg, 0.45 mmol) and isopropyl(2,2,2-trifluoroethyl)cyanamide (75 mg, 0.45 mmol) in 1,4-Dioxane (10 mL) was added ZnCl₂ (246 mg, 1.80 mmol). The mixture was stirred at 90° C. for 16 h under N₂. The mixture was filtered and the filtrate was purified by prep-HPLC to yield title compound (30 mg). ¹H NMR (400 MHz, CDCl₃) δ 8.04 (s, 1H), 7.45-7.31 (m, 2H), 7.08-6.91 (m, 2H), 5.22 (s, 2H), 4.09-4.04 (m, 1H), 3.93-3.85 (m, 4H), 3.80 (q, J=9.2 Hz, 2H), 3.28-3.15 (m, 4H), 1.94 (q, J=4.4 Hz, 2H), 1.74 (q, J=4.3 Hz, 2H), 1.14 (d, J=6.8 Hz, 6H). LCMS (EST): m/z, 519.4 [M+H]⁺.

### Example 227. Preparation of Compound E163

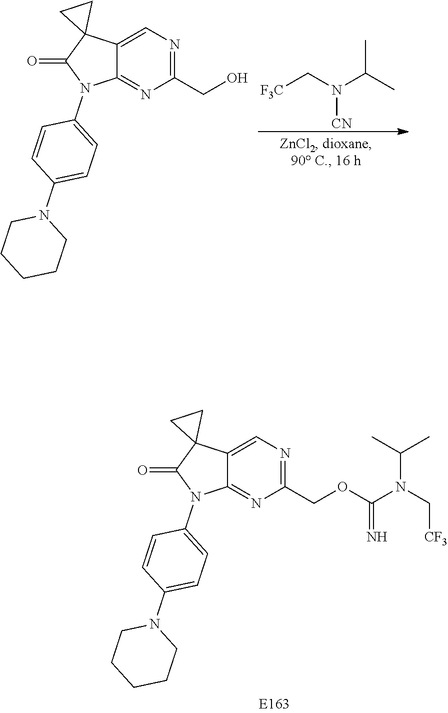 [SMILES: O=C1N(c2ccc(N3CCCCC3)cc2)c2nc(CO)ncc2C12CC2 ; CC(C)N(C#N)CC(F)(F)F ; CC(C)N(CC(F)(F)F)C(=N)OCc1ncc2c(n1)N(c1ccc(N3CCCCC3)cc1)C(=O)C21CC1]

To a solution of 2′-(hydroxymethyl)-7′-[4-(1-piperidyl)phenyl]spiro[cyclopropane-1,5′-pyrrolo[2,3-d]pyrimidine]-6′-one (136 mg, 0.39 mmol) and isopropyl(2,2,2-trifluoroethyl)cyanamide (64 mg, 0.39 mmol) in 1,4-Dioxane (10 mL) was added ZnCl₂ (212 mg, 1.55 mmol). The mixture was stirred at 90° C. for 16 h under N₂. The mixture was filtered and the filtrate was purified by prep-HPLC to yield title compound (32 mg). ¹H NMR (400 MHz, CDCl₃) δ 8.02 (s, 1H), 7.36-7.29 (m, 2H), 7.02-6.94 (m, 2H), 5.22 (s, 2H), 4.10-4.04 (m, 1H), 3.80 (q, J=9.2 Hz, 2H), 3.29-3.13 (m, 4H), 1.94 (q, J=4.4 Hz, 2H), 1.73 (d, J=4.0 Hz, 2H), 1.61 (q, J=5.6, 4.2 Hz, 2H), 1.14 (d, J=6.8 Hz, 6H). LCMS (ESI): m/z, 517.3 [M+H]⁺.

### Example 228. Preparation of Compound E164

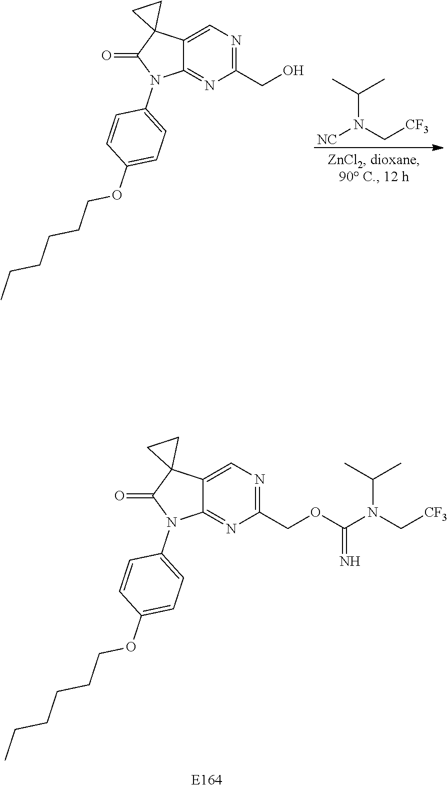

To a solution of 7′-(4-(hexyloxy)phenyl)-2′-(hydroxymethyl)spiro[cyclopropane-1,5′-pyrrolo[2,3-d]pyrimidin]-6′(7′H)-one (108 mg, 0.29 mmol) and isopropyl(2,2,2-trifluoroethyl)cyanamide (146 mg, 0.88 mmol) in anhydrous 1,4-dioxane (3 mL) was added dichlorozine (1 M, 1.18 mL) at 25° C. The mixture was stirred at 90° C. for 12 hours. The mixture was filtered and the filtrate was concentrated. The residue was purified by prep-HPLC to afford title compound (50 mg). ¹H NMR (400 MHz, CDCl₃) δ 8.04 (s, 1H), 7.39-7.37 (m, 2H), 7.00-6.97 (m, 2H), 5.22 (s, 2H), 4.10-4.03 (m, 1H), 3.99 (t, J=6.4 Hz, 2H), 3.83-3.76 (m, 2H), 1.96-1.93 (m, 2H), 1.83-1.78 (m, 2H), 1.76-1.73 (m, 2H), 1.51-1.43 (m, 2H), 1.37-1.32 (m, 4H), 1.25 (s, 1H), 1.14 (d, J=6.8 Hz, 6H), 0.93-0.89 (m, 3H). LCMS (ESI): m/z, 534.3 [M+H]⁺

### Example 229. Preparation of Compound E165

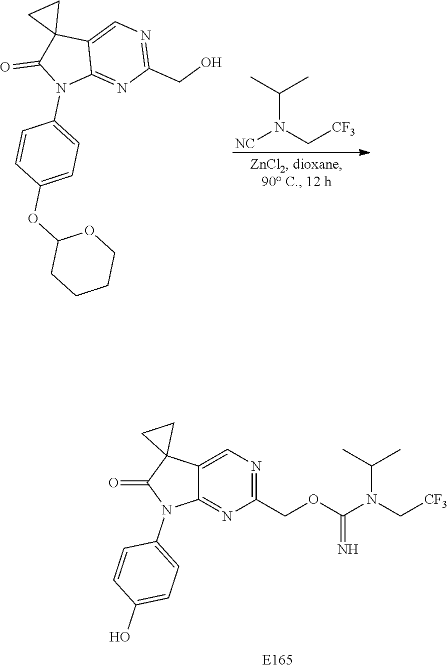

To a solution of 2′-(hydroxymethyl)-7′-(4-((tetrahydro-2H-pyran-2-yl)oxy)phenyl)spiro[cyclopropane-1,5′-pyrrolo[2,3-d]pyrimidin]-6′(7′H)-one (108 mg, 0.29 mmol) and isopropyl(2,2,2-trifluoroethyl)cyanamide (147 mg, 0.88 mmol) in anhydrous 1,4-dioxane (3 mL) was added dichlorozinc (1 M, 1.18 mL) at 25° C. The mixture was stirred at 90° C. for 12 hours. The mixture was filtered. The residue was purified by prep-HPLC to afford title compound (50 mg). ¹H NMR (400 MHz, CDCl₃) δ 9.83 (s, 1H), 9.37 (s, 1H), 8.37 (s, 1H), 7.24-7.22 (m, 2H), 6.89-6.87 (m, 2H), 5.59 (s, 2H), 4.33 (s, 2H), 4.18-4.11 (m, 2H), 1.99-1.96 (m, 2H), 1.76-1.73 (m, 2H), 1.16 (d, J=6.8 Hz, 6H). LCMS (ESI): 450.3 [M+H]⁺.

### Example 230. Preparation of Compound E166

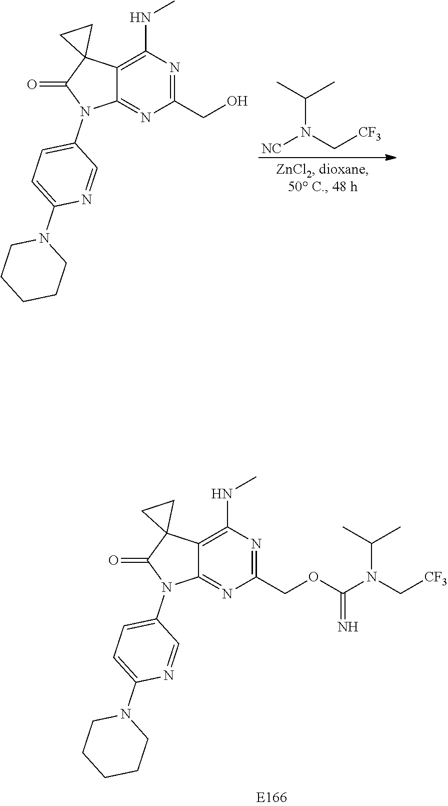 [SMILES: CNc1nc(CO)nc2c1C1(CC1)C(=O)N2c1ccc(N2CCCCC2)nc1 ; CC(C)N(C#N)CC(F)(F)F ; CNc1nc(COC(=N)N(CC(F)(F)F)C(C)C)nc2c1C1(CC1)C(=O)N2c1ccc(N2CCCCC2)nc1]

To a solution of 2′-(hydroxymethyl)-4′-(methylamino)-7′-(6-(piperidin-1-yl)pyridin-3-yl)spiro[cyclopropane-1,5′-pyrrolo[2,3-d]pyrimidin]-6′(7′H)-one (100 mg, 0.26 mmol) and isopropyl(2,2,2-trifluoroethyl)cyanamide (131 mg, 0.78 mmol) in anhydrous 1,4-dioxane (2.5 mL) was added dichlorozinc (1 M, 1.05 mL) at 25° C. The mixture was stirred at 50° C. for 48 hours. The mixture was filtered. The residue was purified by prep-HPLC to afford title compound (10.7 mg). ¹H NMR (400 MHz, CDCl₃) δ 8.24 (d, J=2.8 Hz, 1H), 7.51 (dd, J=2.8, 9.2 Hz, 1H), 6.69 (d, J=9.2 Hz, 1H), 5.07 (s, 2H), 4.16-4.11 (m, 1H), 3.88-3.81 (m, 2H), 3.58-3.57 (m, 4H), 3.02 (d, J=4.8 Hz, 3H), 1.81-1.64 (m, 6H), 1.33-1.22 (m, 4H), 1.17 (d, J=6.8 Hz, 6H). LCMS (ESI): 547.3 [M+H]⁺.

### Example 231. Preparation of Compound E167

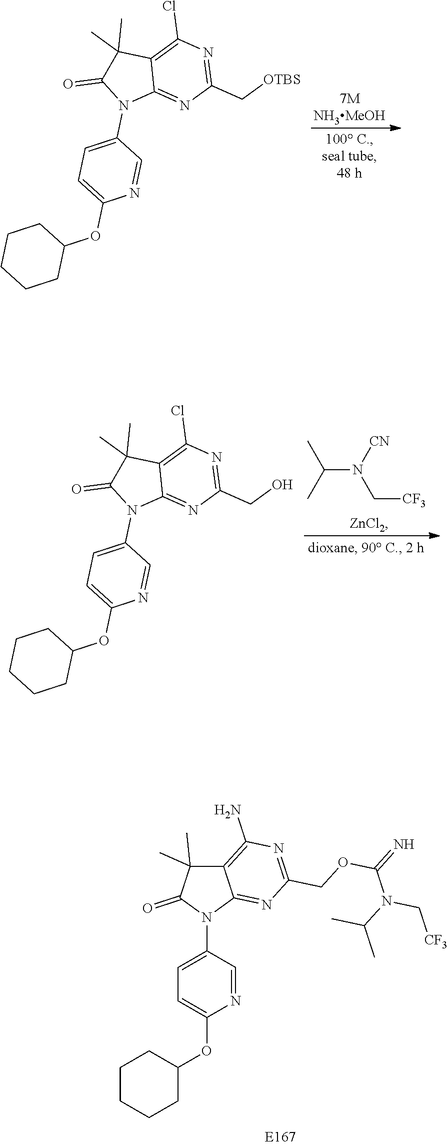

1. Synthesis of compound 4-amino-7-(6-(cyclohexyloxy)pyridin-3-yl)-2-(hydroxymethyl)-5,5-dimethyl-5,7-dihydro-6H-pyrrolo[2,3-d]pyrimidin-6-one. A mixture of 2-(((tert-butyldimethylsilyl)oxy)methyl)-4-chloro-7-(6-(cyclohexyloxy)pyridin-3-yl)-5,5-dimethyl-5,7-dihydro-6H-pyrrolo[2,3-d]pyrimidin-6-one (500 mg, 0.580 mmol) in NH₃/MeOH (1.1 mL, 7 M) was stirred at 100° C. for 48 hours under sealed tub. The mixture was concentrated. The residue was purified by column chromatography to afford title compound (100 mg). LCMS (ESI): m/z, 384.2 [M+H]⁺.

2. Synthesis of compound E167. To a solution of 4-amino-7-(6-(cyclohexyloxy)pyridin-3-yl)-2-(hydroxymethyl)-5,5-dimethyl-5,7-dihydro-6H-pyrrolo[2,3-d]pyrimidin-6-one (90 mg, 0.235 mmol), isopropyl(2,2,2-trifluoroethyl)cyanamide (117 mg, 0.704 mmol) in Dioxane (3 mL) was added ZnCl₂ (128 mg, 0.938 mmol). The mixture was stirred at 90° C. for 2 hours. The mixture was concentrated. The residue was purified by preparative HPLC to afford title compound (30 mg). ¹H NMR (400 MHz, CDCl₃) δ 8.25 (d, J=2.4 Hz, 1H), 7.65 (dd, J=2.4, 8.8 Hz, 1H), 6.77 (d, J=8.8 Hz, 1H), 5.09-5.03 (m, 3H), 4.79 (s, 2H), 4.13-4.06 (m, 1H), 3.87-3.81 (m, 1H), 2.04-2.00 (m, 2H), 1.83-1.78 (m, 2H), 1.53 (s, 6H), 1.50-1.33 (m, 6H), 1.16 (d, J=6.8 Hz, 6H). LCMS (ESI): m/z, 550.3 [M+H]⁺.

### Example 232. Preparation of Compound E168

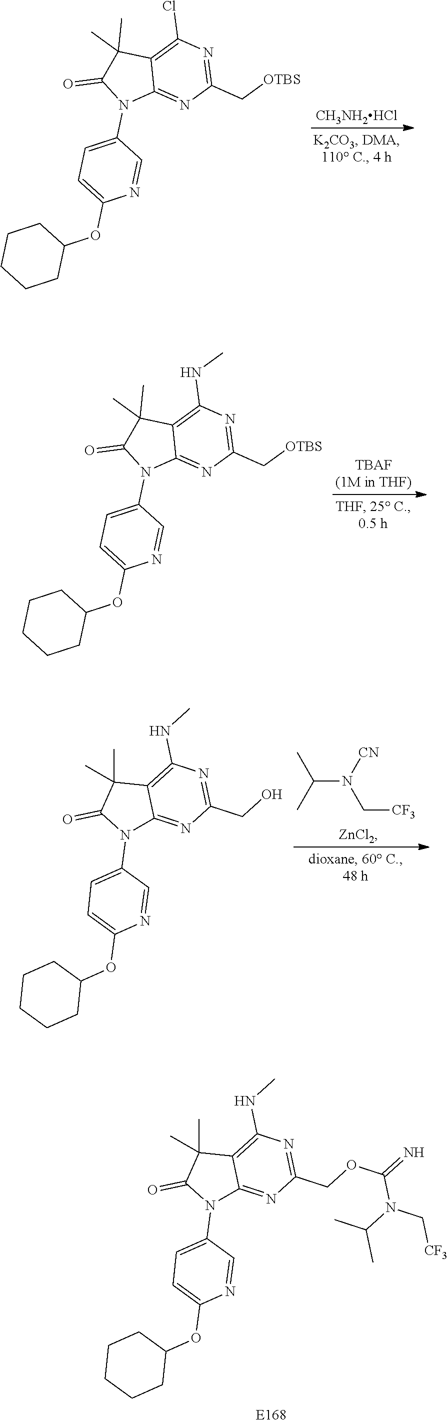

1. Synthesis of compound 2-(((tert-butyldimethylsilyl)oxy)methyl)-7-(6-(cyclohexyloxy)pyridin-3-yl)-5,5-dimethyl-4-(methylamino)-5,7-dihydro-6H-pyrrolo[2,3-d]pyrimidin-6-one. To a solution of 2-(((tert-butyldimethylsilyl)oxy)methyl)-4-chloro-7-(6-(cyclohexyloxy)pyridin-3-yl)-5,5-dimethyl-5,7-dihydro-6H-pyrrolo[2,3-d]pyrimidin-6-one (500 mg, 0.580 mmol), methanamine hydrochloride (97 mg, 1.45 mmol) in DMA (10 mL) was added K₂CO₃ (200 mg, 1.45 mmol). The mixture was stirred at 110° C. for 4 hours. The mixture was diluted with EA, washed with sat.aq. NaCl, dried over Na₂SO₄, filtered and concentrated to afford title compound (230 mg crude). LCMS (ESI): m/z, 512.3 [M+H]⁺.

2. Synthesis of compound 7-(6-(cyclohexyloxy)pyridin-3-yl)-2-(hydroxymethyl)-5,5-dimethyl-4-(methylamino)-5,7-dihydro-6H-pyrrolo[2,3-d]pyrimidin-6-one. To a solution of 2-(((tert-butyldimethylsilyl)oxy)methyl)-7-(6-(cyclohexyloxy)pyridin-3-yl)-5,5-dimethyl-4-(methylamino)-5,7-dihydro-6H-pyrrolo[2,3-d]pyrimidin-6-one (230 mg, 0.449 mmol) in THF (5 mL) was added TBAF (1.35 mL, 1.35 mmol, 1M in THF). The mixture was stirred at 25° C. for 0.5 hour. The mixture was concentrated. The residue was purified by column chromatography to afford title compound (160 mg). LCMS (EST): m/z, 398.2 [M+H]⁺.

3. Synthesis of compound E168. To a solution of isopropyl(2,2,2-trifluoroethyl)cyanamide (125 mg, 0.754 mmol), 7-(6-(cyclohexyloxy)pyridin-3-yl)-2-(hydroxymethyl)-5,5-dimethyl-4-(methylamino)-5,7-dihydro-6H-pyrrolo[2,3-d]pyrimidin-6-one (100 mg, 0.251 mmol) in dioxane (1 mL) was added ZnCl₂ (137 mg, 1.01 mmol). The mixture was stirred at 60° C. for 48 hours. The mixture was filtered. The filtrate was purified by pre-HPLC to afford title compound (6 mg). ¹H NMR (400 MHz, CDCl₃) b 8.18 (d, J=2.4 Hz, 1H), 7.23 (dd, J=2.8, 8.8 Hz, 1H), 6.89 (d, J=8.8 Hz, 1H), 6.78 (s, 1H), 5.97 (s, 1H), 5.07-5.01 (m, 1H), 4.97 (s, 2H), 4.00-3.93 (m, 3H), 2.93 (d, J=4.4 Hz, 3H), 2.01-1.97 (m, 2H), 1.78-1.75 (m, 2H), 1.60-1.49 (m, 2H), 1.46 (s, 6H), 1.42-1.31 (m, 4H), 1.05 (d, J=6.4 Hz, 6H). LCMS (ESI): m/z, 564.4 [M+H]⁺.

### Example 233. Preparation of Compound E169

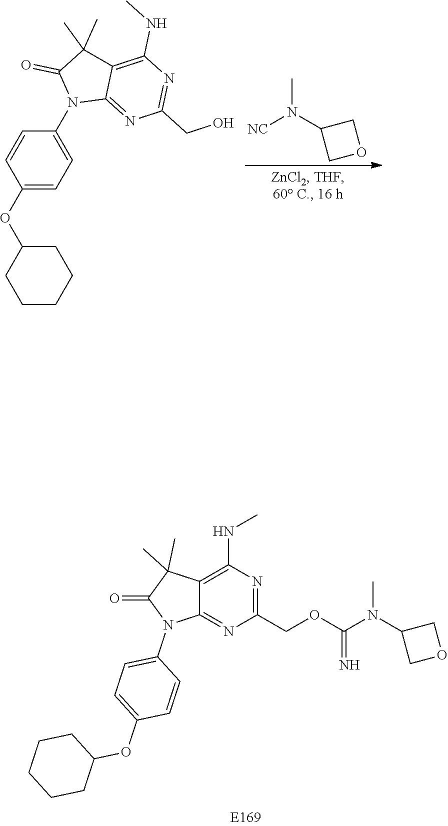

To a solution of 7-(4-(cyclohexyloxy)phenyl)-2-(hydroxymethyl)-5,5-dimethyl-4-(methylamino)-5,7-dihydro-6H-pyrrolo[2,3-d]pyrimidin-6-one (80 mg, 0.20 mmol) and methyl(oxetan-3-yl)cyanamide (68 mg, 0.61 mmol) was added ZnCl₂ (110 mg, 0.81 mmol). The mixture was stirred at 60° C. for 16 h. The mixture was filtered. The residue was purified by prep-HPLC to give title compound (7.9 mg). ¹H NMR (400 MHz, CDCl₃) δ 7.36 (d, J=8.8 Hz, 2H), 6.95 (d, J=9.2 Hz, 2H), 5.30-5.07 (m, 2H), 4.38 (d, J 5.2 Hz, 1H), 4.26 (dt, J 4.8, 8.8 Hz, 1H), 3.80-3.64 (m, 2H), 3.61-3.42 (m, 3H), 3.08 (d, J=4.8 Hz, 3H), 2.73 (s, 3H), 2.04-1.95 (m, 2H), 1.85-1.78 (m, 2H), 1.60-1.52 (m, 3H), 1.48 (d, J=1.6 Hz, 6H), 1.41-1.32 (m, 3H). LCMS (ESI): m/z, 509.3 [M+H]⁺.

### Example 234. Preparation of Compound E170

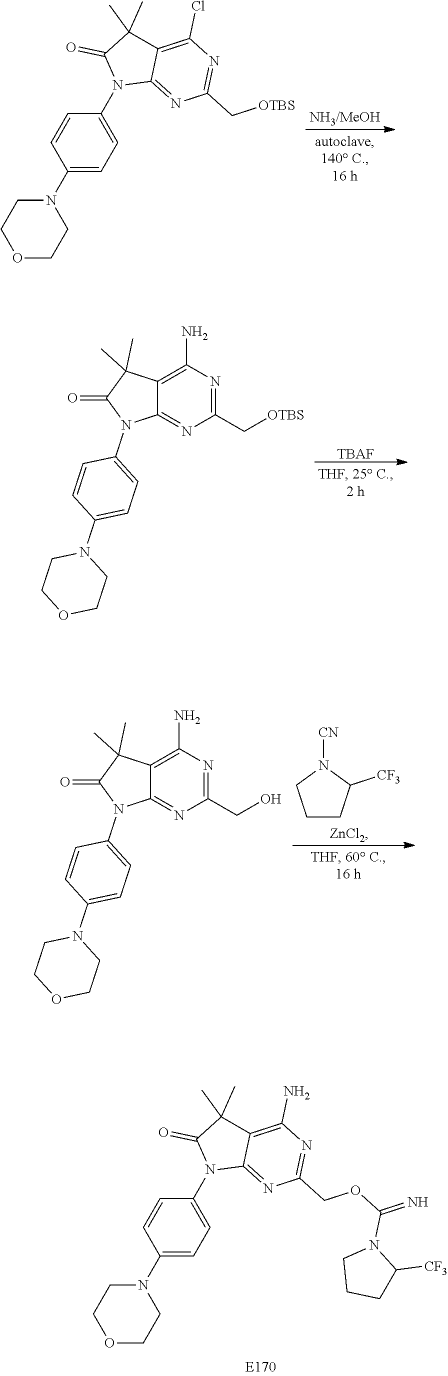

1. Synthesis of compound 4-amino-2-(((tert-butyldimethylsilyl)oxy)methyl)-5,5-dimethyl-7-(4-morpholinophenyl)-5,7-dihydro-6H-pyrrolo[2,3-d]pyrimidin-6-one. A solution of 2-(((tert-butyldimethylsilyl)oxy)methyl)-4-chloro-5,5-dimethyl-7-(4-morpholinophenyl)-5,7-dihydro-6H-pyrolo[2,3-d]pyrimidin-6-one (1 g, 1.99 mmol) in NH₃/MeOH (20 mL, 7 M) was stirred at 140° C. for 16 h in autoclave. The mixture was concentrated to yield title compound (960 mg crude). LCMS (ESI): m/z, 484.3 [M+H]⁺.

2. Synthesis of compound 4-amino-2-(hydroxymethyl)-5,5-dimethyl-7-(4-morpholinophenyl)-5,7-dihydro-6H-pyrrolo[2,3-d]pyrimidin-6-one. To a solution of 4-amino-2-(((tert-butyldimethylsilyl)oxy)methyl)-5,5-dimethyl-7-(4-morpholinophenyl)-5,7-dihydro-6H-pyrrolo[2,3-d]pyrimidin-6-one (960 mg, 1.98 mmol) in THF (10 mL) was added TBAF (2.34 mL, 1 M). The mixture was stirred at 25° C. for 2 h. Water (50 mL) was added and extracted with EtOAc. The combined organic phases were washed with brine, dried over anhydrous Na₂SO₄, filtered and concentrated. The residue was purified by column chromatography to give title compound (200 mg). ¹H NMR (400 MHz, CDCl₃) δ 7.41-7.30 (m, 2H), 7.03-6.94 (m, 2H), 4.80 (s, 2H), 4.53 (s, 2H), 3.94-3.81 (m, 4H), 3.60 (s, 1H), 3.26-3.17 (m, 4H), 1.53 (s, 6H). LCMS (ESI): m/z, 370.2 [M+H]⁺.

3. Synthesis of compound E170. To a solution of 4-amino-2-(hydroxymethyl)-5,5-dimethyl-7-(4-morpholinophenyl)-5,7-dihydro-6H-pyrrolo[2,3-d]pyrimidin-6-one (50 mg, 0.14 mmol) and 2-(trifluoromethyl)pyrrolidine-1-carbonitrile (67 mg, 0.41 mmol) in THF (3 mL) was added ZnCl₂ (74 mg, 0.54 mmol). The mixture was stirred at 60° C. for 16 h under N₂. The mixture was filtered. The filtrate was purified by prep-HPLC to yield title compound (12.3 mg). ¹H NMR (400 MHz, CDCl₃) (δ 7.44-7.30 (m, 2H), 7.03-6.83 (m, 2H), 5.18 (d, J=14.4 Hz, 1H), 5.00 (d, J=14.4 Hz, 1H), 4.81 (s, 2H), 4.46-4.41 (m, 1H), 3.94-3.79 (m, 4H), 3.54-3.30 (m, 2H), 3.26-3.09 (m, 4H), 2.06 (t, J=9.6 Hz, 2H), 1.94-1.81 (m, 2H), 1.51 (s, 6H). LCMS (ESI): m/z, 534.3 [M+H]⁺.

### Example 235. Preparation of Compound E171

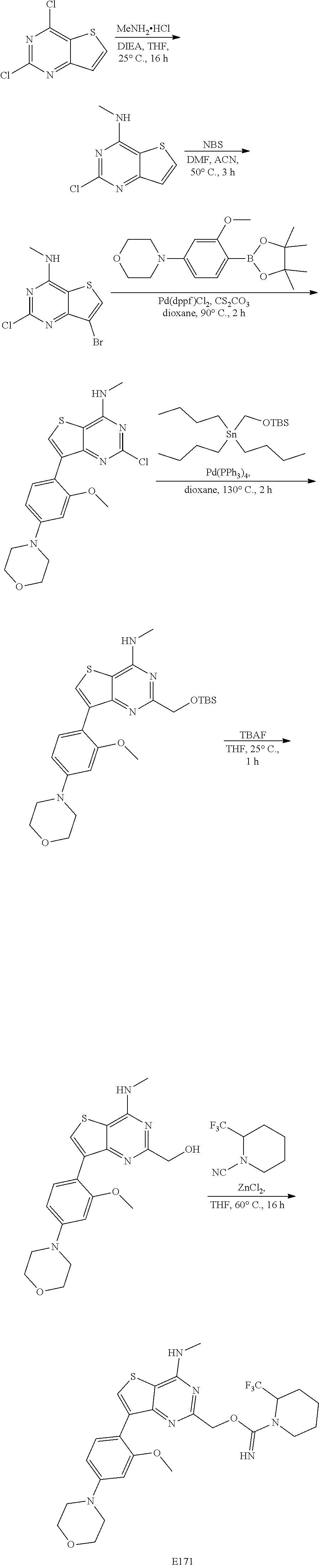

1. Synthesis of compound 2-chloro-N-methyl-thieno[3,2-d]pyrimidin-4-amine. To a solution of 2,4-dichlorothieno[3,2-d]pyrimidine (1.0 g, 4.88 mmol) and DIEA (1.89 g, 14.6 mmol, 2.42 mL) in THF (10 mL) was added Methylamine hydrochloride (329 mg, 4.88 mmol) at 0° C. and stirred at 25° C. for 16 h. The mixture was concentrated to yield a residue which was treated with water (20 mL) and stirred for 10 min. The precipitated solid was filtered and collected. The solid was triturated with EtOAc to give title compound (620 mg). LCMS (EST): m/z, 200.0 [M+H]⁺.

2. Synthesis of compound 7-bromo-2-chloro-N-methyl-thieno[3,2-d]pyrimidin-4-amine. To a solution of 2-chloro-N-methyl-thieno[3,2-d]pyrimidin-4-amine (600 mg, 3.01 mmol) in ACN (10 mL) and DMF (10 mL) was added NBS (802 mg, 4.51 mmol). The mixture was stirred at 50° C. for 3 h. The mixture was poured into water. The precipitated solid was collected and dried to give title compound (650 mg). ¹H NMR (400 MHz, CDCl₃) δ 7.72 (s, 1H), 5.23 (s, 1H), 3.24 (d, J=4.0 Hz, 3H).

3. Synthesis of compound 2-chloro-7-(2-methoxy-4-morpholino-phenyl)-N-methyl-thieno[3,2-d]pyrimidin-4-amine. To a solution of 4-(3-methoxy-4-(4,4,5,5-tetramethyl-1,3,2-dioxaborolan-2-yl)phenyl)morpholine (722 mg, 2.26 mmol) and 7-bromo-2-chloro-N-methyl-thieno[3,2-d]pyrimidin-4-amine (630 mg, 2.26 mmol) in 1,4-dioxane (15 mL) and H₂O (3 mL) were added Pd(dppf)Cl₂ (166 mg, 0.23 mmol) and Cs₂CO₃ (1.47 g, 4.52 mmol). The mixture was stirred at 90° C. for 2 h under N₂. The mixture was filtered, water was added to the filtrate and extracted with EtOAc. The combined organic phases were washed with brine, dried over anhydrous Na₂SO₄, filtered and concentrated. The residue was purified by column chromatography to give title compound (650 mg). ¹H NMR (400 MHz, CDCl₃) δ 7.90 (s, 1H), 7.82 (d, J=8.5 Hz, 1H), 6.63 (dd, J=2.4, 8.4 Hz, 1H), 6.58-6.51 (m, 1H), 5.06 (d, J=5.2 Hz, 1H), 3.91-3.85 (m, 4H), 3.83 (s, 3H), 3.28-3.18 (m, 7H). LCMS (ESI): m/z, 391.1 [M+H]⁺

4. Synthesis of compound 2-(((tert-butyldimethylsilyl)oxy)methyl)-7-(2-methoxy-4-morpholinophenyl)-N-methylthieno[3,2-d]pyrimidin-4-amine. To a solution of 2-chloro-7-(2-methoxy-4-morpholinophenyl)-N-methylthieno[3,2-d]pyrimidin-4-amine (560 mg, 1.43 mmol) in 1,4-Dioxane (10 mL) were added tert-butyl-dimethyl-(tributylstannylmethoxy)silane (686 mg, 1.58 mmol) and Pd(PPh₃)₄ (166 mg, 0.14 mmol). The mixture was stirred at 130° C. for 2 h in microwave under N₂. The solvent was removed. The residue was purified by column chromatography to give title compound (450 mg). LCMS (ESI): m/z, 501.2 [M+H]⁺. [001279]5. Synthesis of compound (7-(2-methoxy-4-morpholinophenyl)-4-(methylamino)thieno[3,2-d]pyrimidin-2-yl)methanol. To a solution of 2-(((tert-butyldimethylsilyl)oxy)methyl)-7-(2-methoxy-4-morpholinophenyl)-N-methylthieno[3,2-d]pyrimidin-4-amine (450 mg, 0.9 mmol) in THF (3.50 mL) was added TBAF (1 M, 4.49 mL).The mixture was stirred at 25° C. for 1 h. Water (20 mL) was added and extracted with EtOAc. The combined organic phases were washed with water, brine, dried over anhydrous Na₂SO₄, filtered and concentrated to give title compound (300 mg). ¹H NMR (400 MHz, CDCl₃) δ 7.89 (s, 1H), 7.81 (d, J=8.4 Hz, 1H), 6.62 (dd, J=2.4, 8.4 Hz, 1H), 6.56 (d, J=2.4 Hz, 1H), 4.99 (s, 1H), 4.75-4.68 (m, 2H), 4.18 (s, 1H), 3.93-3.86 (m, 4H), 3.83 (s, 3H), 3.26-3.22 (m, 7H). LCMS (ESI): m/z, 387.2 [M+H]⁺.

6. Synthesis of compound E171. To a solution of (7-(2-methoxy-4-morpholinophenyl)-4-(methylamino)thieno[3,2-d]pyrimidin-2-yl)methanol (100 mg, 0.26 mmol) and 2-(trifluoromethyl)piperidine-1-carbonitrile (138 mg, 0.77 mmol) in THF (4 mL) was added ZnCl₂ (141 mg, 1.04 mmol). The mixture was stirred at 60° C. for 16 h under N₂. The mixture was filtered. The residue was purified by prep-HPLC to give title compound (24.5 mg). ¹H NMR (400 MHz, CDCl₃) δ 7.94-7.86 (m, 2H), 6.61 (dd, J=2.4, 8.4 Hz, 1H), 6.55 (d, J=2.4 Hz, 1H), 5.28-5.09 (m, 2H), 5.01-4.87 (m, 2H), 4.08 (d, J=12.8 Hz, 1H), 3.93-3.85 (m, 4H), 3.82 (s, 3H), 3.28-3.16 (m, 7H), 3.04 (t, J=12.8 Hz, 1H), 2.00-1.90 (m, 1H), 1.77-1.61 (m, 4H), 1.58-1.42 (m, 1H). LCMS (ESI): m/z, 565.32 [M+H]⁺.

### Example 236. Preparation of Compound E172

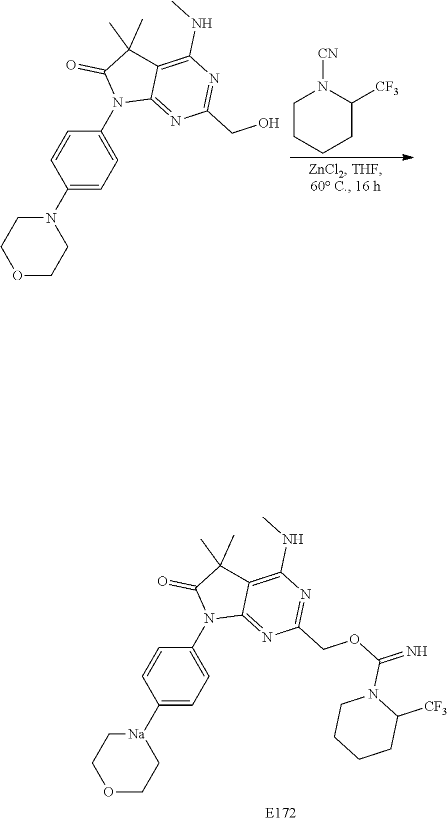

To a solution of 4-amino-2-(hydroxymethyl)-5,5-dimethyl-7-(4-morpholinophenyl)-5,7-dihydro-6H-pyrrolo[2,3-d]pyrimidin-6-one (50 mg, 0.14 mmol) and 2-(trifluoromethyl)piperidine-1-carbonitrile (72 mg, 0.41 mmol) in THF (3 mL) was added ZnCl₂ (74 mg, 0.54 mmol). The mixture was stirred at 60° C. for 16 h under N₂. The mixture was filtered and the filtrate was concentrated. The residue was purified by prep-HPLC to yield title compound (39.5 mg). ¹H NMR (400 MHz, CDCl₃) δ 7.35 (d, J=8.8 Hz, 2H), 6.96 (d, J=9.2 Hz, 2H), 5.13-4.87 (m, 4H), 4.80 (dd, J=5.6, 9.6 Hz, 1H), 3.98-3.78 (m, 5H), 3.24-3.12 (m, 4H), 3.08-2.93 (m, 1H), 1.97-1.87 (m, 1H), 1.77-1.63 (m, 4H), 1.51 (s, 6H), 1.48-1.39 (m, 1H). LCMS (ESI): m/z, 548.3 [M+H]⁺.

### Example 237. Preparation of Compound E173

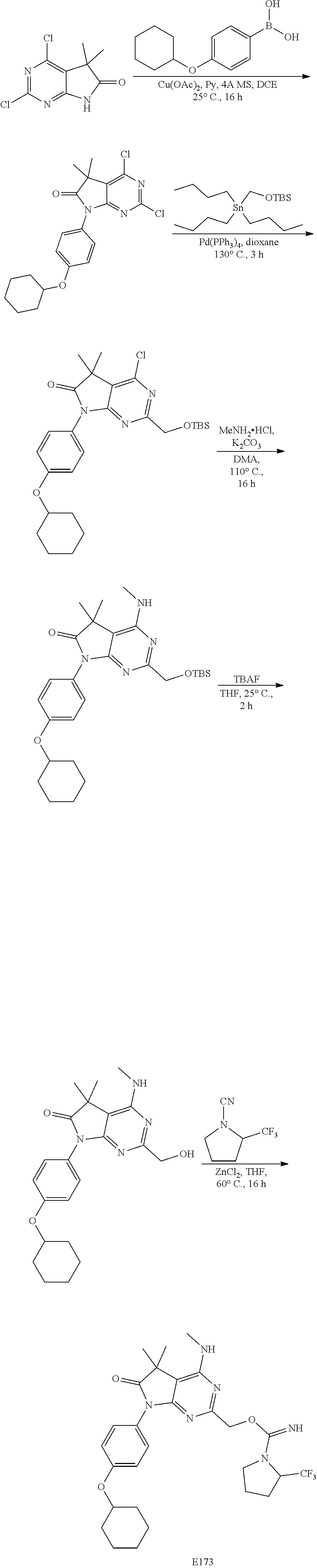

1. Synthesis of compound 2,4-dichloro-7-(4-(cyclohexyloxy)phenyl)-5,5-dimethyl-5,7-dihydro-6H-pyrrolo[2,3-d]pyrimidin-6-one. To a solution of 2,4-dichloro-5,5-dimethyl-5,7-dihydro-6H-pyrrolo[2,3-d]pyrimidin-6-one (1.0 g, 4.31 mmol) and (4-(cyclohexyloxy)phenyl)boronic acid (1.90 g, 8.62 mmol) in DCE (80 mL) were added Cu(OAc)₂ (939 mg, 5.17 mmol), Py (1.05 mL) and 4A MS (500 mg). The mixture was stirred at 25° C. for 16 h under O₂. The mixture was filtered and the filter cake was washed with EtOAc. The filtrate was concentrated to yield a residue which was purified by column chromatography to give title compound (1.4 g). ¹H NMR (400 MHz, CDCl₃) δ 7.33-7.27 (m, 2H), 7.07-6.94 (m, 2H), 4.28 (td, J=4.4, 8.8, Hz, 1H), 2.03-1.95 (m, 2H), 1.86-1.80 (m, 2H), 1.60 (s, 6H), 1.58 (d, J=4.0 Hz, 2H), 1.45-1.30 (m, 4H). LCMS (ESI): m/z, 506.0 [M+H]⁺.

2. Synthesis of compound 2-(((tert-butyldimethylsilyl)oxy)methyl)-4-chloro-7-(4-(cyclohexyloxy)phenyl)-5,5-dimethyl-5,7-dihydro-6H-pyrrolo[2,3-d]pyrimidin-6-one. To a solution of 2,4-dichloro-7-(4-(cyclohexyloxy)phenyl)-5,5-dimethyl-5,7-dihydro-6H-pyrrolo[2,3-d]pyrimidin-6-one (1.4 g, 3.45 mmol) in 1,4-Dioxane (15 mL) were added tert-butyl-dimethyl-(tributylstannylmethoxy)silane (1.50 g, 3.45 mmol) and Pd(PPh₃)₄ (398 mg, 0.34 mmol). The mixture was stirred at 130° C. for 3 h under N₂ in microwave. The solvent was removed. The residue was purified by column chromatography to give title compound (1.0 g).

3. Synthesis of compound 2-(((tert-butyldimethylsilyl)oxy)methyl)-7-(4-(cyclohexyloxy)phenyl)-5,5-dimethyl-4-(methylamino)-5,7-dihydro-6H-pyrrolo[2,3-d]pyrimidin-6-one. To a solution of 2-(((tert-butyldimethylsilyl)oxy)methyl)-4-chloro-7-(4-(cyclohexyloxy)phenyl)-5,5-dimethyl-5,7-dihydro-6H-pyrrolo[2,3-d]pyrimidin-6-one (400 mg, 0.77 mmol) in DMA (10 mL) were added MeNH₂·HCl (157 mg, 2.32 mmol) and K₂CO₃ (428 mg, 3.10 mmol). The mixture was stirred at 110° C. for 16 h. The mixture was filtered and the filtrate was treated with water, extracted with EtOAc. The combined organic phases were washed with water, brine, dried over anhydrous Na₂SO₄, filtered and concentrated to give title compound (400 mg crude). LCMS (ESI): m/z, 511.3 [M+H]⁺.

4. Synthesis of compound 7-(4-(Cyclohexyloxy)phenyl)-2-(hydroxymethyl)-5,5-dimethyl-4-(methylamino)-5,7-dihydro-6H-pyrrolo[2,3-d]pyrimidin-6-one. To a solution of 2-(((tert-butyldimethylsilyl)oxy)methyl)-7-(4-(cyclohexyloxy)phenyl)-5,5-dimethyl-4-(methylamino)-5,7-dihydro-6H-pyrrolo[2,3-d]pyrimidin-6-one (520 mg, 1.02 mmol) in THF (3 mL) was added TBAF (5.09 mL, 1 M in THF) and stirred at 25° C. for 2 h. Water was added and extracted with EtOAc. The combined organic phases were washed with water, brine, dried over anhydrous Na₂SO₄, filtered and concentrated. The residue was purified by column chromatography to give title compound (180 mg). ¹H NMR (400 MHz, CDCl₃) 3 7.38-7.28 (m, 2H), 7.03-6.92 (m, 2H), 4.55 (s, 2H), 4.45 (d, J=5.0 Hz, 1H), 4.29-4.23 (m, 1H), 3.78 (s, 1H), 3.13 (d, J=4.8 Hz, 3H), 2.02 (s, 2H), 1.86-1.77 (m, 2H), 1.59 (s, 2H), 1.51 (s, 6H), 1.44-1.25 (m, 4H).

5. Synthesis of compound E173. To a solution of 7-(4-(cyclohexyloxy)phenyl)-2-(hydroxymethyl)-5,5-dimethyl-4-(methylamino)-5,7-dihydro-6H-pyrrolo[2,3-d]pyrimidin-6-one (70 mg, 0.18 mmol) and 2-(trifluoromethyl)pyrrolidine-1-carbonitrile (87 mg, 0.53 mmol) in THF (3 mL) was added ZnCl₂ (96 mg, 0.71 mmol) and the mixture was stirred at 60° C. for 16 h. The mixture was filtered. The filtrate was concentrated. The residue was purified by prep-HPLC to give title compound (43 mg). ¹H NMR (400 MHz, CDCl₃) δ 7.40-7.31 (m, 2H), 6.98-6.89 (m, 2H), 5.30-4.95 (m, 2H), 4.49-4.41 (m, 1H), 4.40 (q, J=4.8 Hz, 1H), 4.28-4.22 (m, 1H), 3.54-3.35 (m, 2H), 3.08 (d, J=4.8 Hz, 3H), 2.12-1.96 (m, 4H), 1.96-1.78 (m, 4H), 1.64-1.51 (m, 3H), 1.48 (s, 6H), 1.43-1.29 (m, 3H). LCMS (ESI): m/z, 561.3 [M+H]⁺.

### Example 238. Preparation of Compound E174

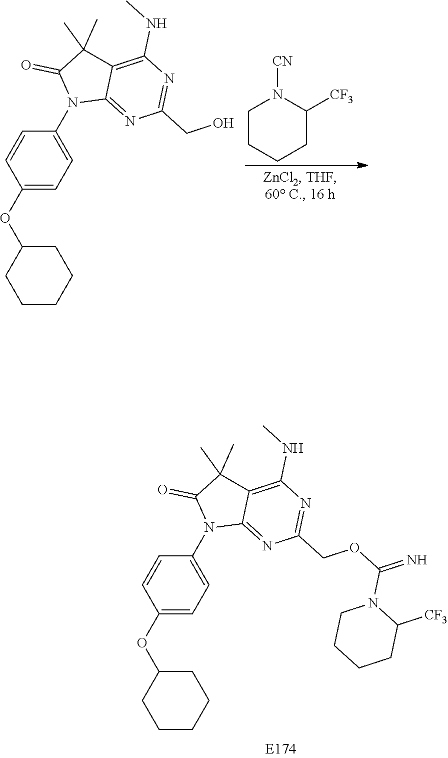

To a solution of 7-(4-(cyclohexyloxy)phenyl)-2-(hydroxymethyl)-5,5-dimethyl-4-(methylamino)-5,7-dihydro-6H-pyrrolo[2,3-d]pyrimidin-6-one (70 mg, 0.17 mmol) and 2-(trifluoromethyl)piperidine-1-carbonitrile (94 mg, 0.53 mmol) in THF (3 mL) was added ZnCl₂ (96 mg, 0.71 mmol). The mixture was stirred at 60° C. for 16 h under N₂. The mixture was filtered. The filtrate was concentrated. The residue was purified by prep-HPLC to yield title compound (43 mg). ¹H NMR (400 MHz, CDCl₃) δ 7.40-7.29 (m, 2H), 6.99-6.89 (m, 2H), 5.12-4.92 (m, 2H), 4.84 (dt, J=7.7, 15.5 Hz, 1H), 4.43 (q, J=4.8 Hz, 1H), 4.27-4.21 (m, 1H), 4.04-3.93 (m, 1H), 3.46-3.37 (m, 1H), 3.36-3.27 (m, 1H), 3.08 (d, J=4.8 Hz, 3H), 3.01 (t, J=13.2 Hz, 1H), 2.06-1.86 (m, 5H), 1.86-1.69 (m, 4H), 1.61-1.51 (m, 3H), 1.48 (s, 6H), 1.42-1.23 (m, 4H). LCMS (ESI): m/z, 575.4 [M+H]⁺.

### Example 239. Preparation of Compound E175

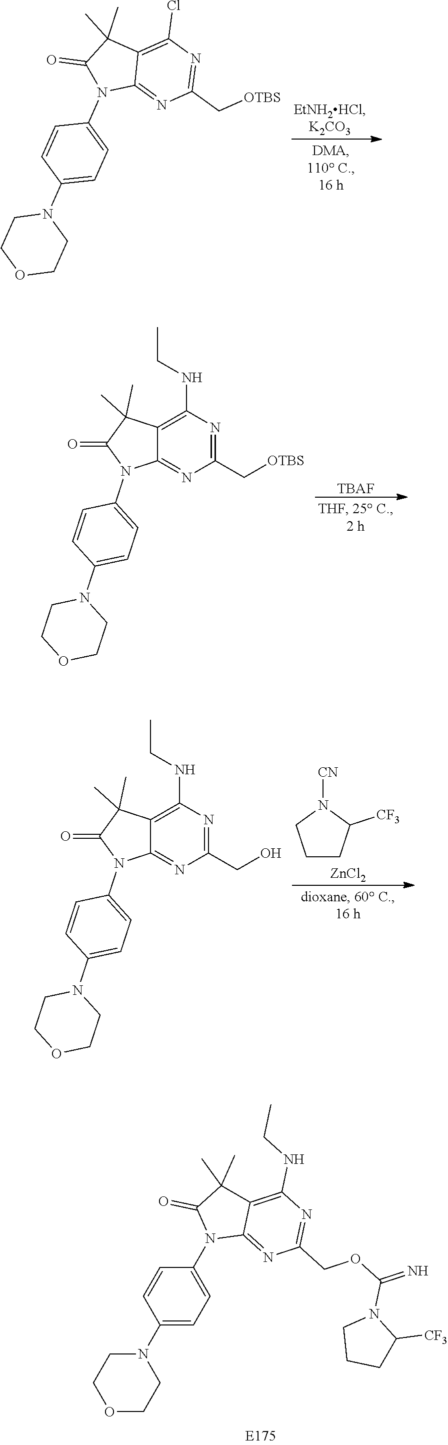

1. Synthesis of compound 2-(((tert-butyldimethylsilyl)oxy)methyl)-4-(ethylamino)-5,5-dimethyl-7-(4-morpholinophenyl)-5,7-dihydro-6H-pyrrolo[2,3-d]pyrimidin-6-one. To a solution of 2-(((tert-butyldimethylsilyl)oxy)methyl)-4-chloro-5,5-dimethyl-7-(4-morpholinophenyl)-5,7-dihydro-6H-pyrrolo[2,3-d]pyrimidin-6-one (360 mg, 0.72 mmol) in DMA (5 mL) were added K₂CO₃ (494 mg, 3.58 mmol) and ethanamine; hydrochloride (175 mg, 2.15 mmol). The mixture was stirred at 110° C. for 16 hours. The mixture was filtered and the filtrate was concentrated to afford title compound (366 mg crude).

2. Synthesis of compound 4-(ethylamino)-2-(hydroxymethyl)-5,5-dimethyl-7-(4-morpholinophenyl)pyrrolo[2,3-d]pyrimidin-6-one. To a solution of 2-(((tert-butyldimethylsilyl)oxy)methyl)-4-(ethylamino)-5,5-dimethyl-7-(4-morpholinophenyl)-5,7-dihydro-6H-pyrrolo[2,3-d]pyrimidin-6-one (366 mg, 0.72 mmol) in THF (5 mL) was added TBAF (1 M, 2.86 mL). The mixture was stirred at 25° C. for 2 hours. Water was added and extracted with EtOAc. The combined organic phases were washed with brine, dried over anhydrous Na₂SO₄, filtered and concentrated. The residue was purified by column chromatography to give title compound (131 mg).

3. Synthesis of compound E175. To a mixture of 4-(ethylamino)-2-(hydroxymethyl)-5,5-dimethyl-7-(4-morpholinophenyl)-5,7-dihydro-6H-pyrrolo[2,3-d]pyrimidin-6-one (131 mg, 0.33 mmol), 2-(trifluoromethyl)pyrrolidine-1-carbonitrile (162 mg, 0.99 mmol) and ZnCl₂ (180 mg, 1.32 mmol) in dioxane (2 mL) was stirred at 60° C. for 16 hours. The reaction was filtered and the filtrate was concentrated to give a residue which was purified by Prep-HPLC to afford title compound (84 mg). ¹H NMR (400 MHz, CDCl₃) δ 7.39-7.33 (m, 2H), 6.99-6.93 (m, 2H), 5.22 (d, J=14.4 Hz, 1H), 5.04 (d, J=14.4 Hz, 1H), 4.58-4.46 (m, 1H), 4.35 (t, J=5.6 Hz, 1H), 3.91-3.83 (m, 4H), 3.67-3.54 (m, 2H), 3.54-3.42 (m, 2H), 3.22-3.17 (m, 4H), 2.23-1.99 (m, 4H), 1.50 (s, 6H), 1.28-1.24 (m, 3H). LCMS (ESI): m/z 562.2 [M+H]⁺.

### Example 240. Preparation of Compound E176

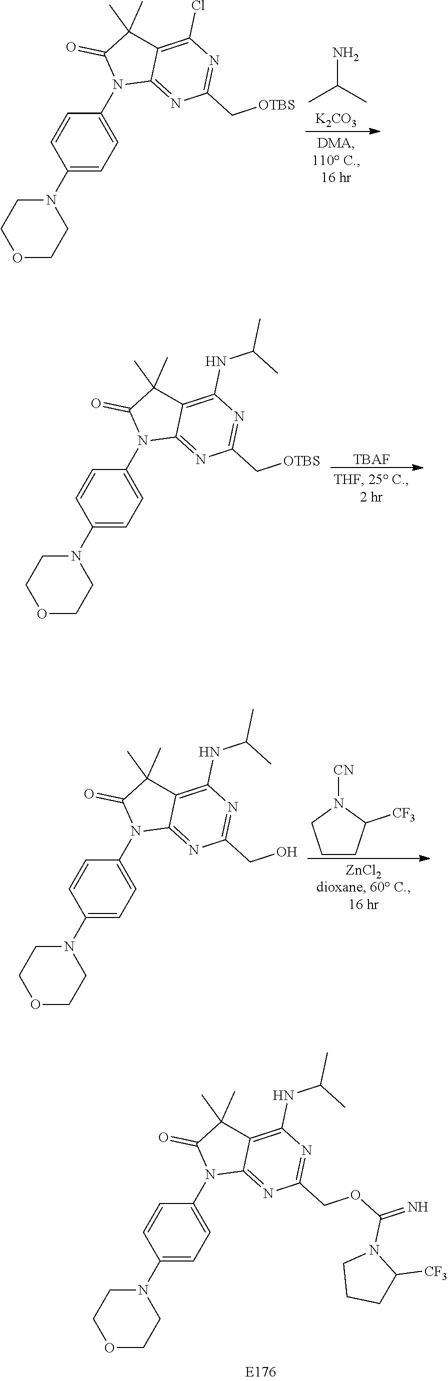

1. Synthesis of 2-(((tert-Butyldimethylsilyl)oxy)methyl)-4-(isopropylamino)-5,5-dimethyl-7-(4-morpholinophenyl)-5,7-dihydro-6H-pyrrolo[2,3-d]pyrimidin-6-one. To a solution of 2-(((tert-butyldimethylsilyl)oxy)methyl)-4-chloro-5,5-dimethyl-7-(4-morpholinophenyl)-5,7-dihydro-6H-pyrrolo[2,3-d]pyrimidin-6-one (360 mg, 0.72 mmol) in DMA (5 mL) were added K₂CO₃ (494 mg, 3.58 mmol) and propan-2-amine (85 mg, 1.43 mmol). The mixture was stirred at 110° C. for 16 hours. The mixture was filtered and concentrated to afford title compound (376 mg crude).

2. Synthesis of 2-(Hydroxymethyl)-4-(isopropylamino)-5,5-dimethyl-7-(4-morpholinophenyl)-5,7-dihydro-6H-pyrrolo[2,3-d]pyrimidin-6-one. To a solution of 2-(((tert-butyldimethylsilyl)oxy)methyl)-4-(isopropylamino)-5,5-dimethyl-7-(4-morpholinophenyl)-5,7-dihydro-6H-pyrrolo[2,3-d]pyrimidin-6-one (376 mg, 0.72 mmol) in THF (5 mL) was added TBAF (1 M, 2.86 mL). The mixture was stirred at 25° C. for 2 hours. Water was added and extracted with EtOAc. The combined organic phases were washed with brine, dried over anhydrous Na₂SO₄, filtered and concentrated. The residue was purified by column chromatography to give title compound (106 mg).

3. Synthesis of (4-(Isopropylamino)-5,5-dimethyl-7-(4-morpholinophenyl)-6-oxo-6,7-dihydro-5H-pyrrolo[2,3-d]pyrimidin-2-yl)methyl 2-(trifluoromethyl)pyrrolidine-1-carbimidate. To a mixture of 2-(hydroxymethyl)-4-(isopropylamino)-5,5-dimethyl-7-(4-morpholinophenyl)-5,7-dihydro-6H-pyrrolo[2,3-d]pyrimidin-6-one (106 mg, 0.26 mmol), 2-(trifluoromethyl)pyrrolidine-1-carbonitrile (127 mg, 0.77 mmol) and ZnCl₂ (140 mg, 1.03 mmol) in dioxane (2 mL) was stirred at 60° C. for 16 hours. The reaction was filtered and concentrated to give a residue which was purified by Prep-HPLC to afford title compound (25 mg). ¹H NMR (400 MHz, CDCl₃) δ 7.40-7.33 (m, 2H), 6.99-6.93 (m, 2H), 5.21 (d, J=14.8 Hz, 1H), 5.01 (d, J=14.8 Hz, 1H), 4.55-4.34 (m, 2H), 4.11 (d, J=7.6 Hz, 1H), 3.92-3.84 (m, 4H), 3.53-3.38 (m, 2H), 3.23-3.16 (m, 4H), 2.12-2.04 (m, 2H), 1.93-1.81 (m, 2H), 1.49 (s, 6H), 1.26 (d, J=6.4 Hz, 6H). LCMS (ESI): m/z 576.4 [M+H]⁺.

### Example 241. Preparation of Compound E177

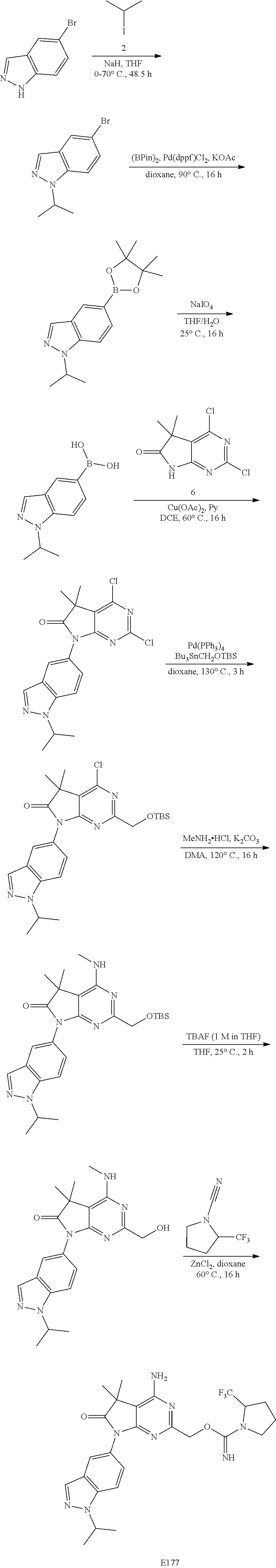

1. Synthesis of 5-Bromo-1-isopropyl-1H-indazole. To a solution of 5-bromo-1H-indazole (10.00 g, 50.75 mmol) in THF (100 mL) was added Sodium hydride (2.44 g, 60.90 mmol) at 0° C. The mixture was stirred at 25° C. for 0.5 h. 2-Iodopropane (9.49 g, 55.83 mmol) was added into the mixture. The reaction mixture was stirred at 70° C. for 48 hours. The reaction mixture was quenched by addition of H₂O at 0° C. The reaction mixture was extracted with EtOAc. The organic layers were washed with brine, dried over Na₂SO₄, filtered and concentrated. The residue was purified by column chromatography to afford title compound (4.70 g). ¹H NMR (400 MHz, CDCl₃) δ 7.94 (s, 1H), 7.86 (dd, J=0.8, 1.6 Hz, 1H), 7.43 (dd, J=2.0, 8.8 Hz, 1H), 7.34-7.32 (m, 1H), 4.86-4.76 (m, 1H), 1.58 (d, J=6.8 Hz, 6H). LCMS (ESI): m/z, 239.0, 241.0 [M+H]⁺.

2. Synthesis of 1-Isopropyl-5-(4,4,5,5-tetramethyl-1,3,2-dioxaborolan-2-yl)-1H-indazole. A mixture of 5-bromo-1-isopropyl-1H-indazole (2.00 g, 8.36 mmol), AcOK (1.64 g, 16.73 mmol), Pin₂B2 (2.23 g, 8.78 mmol) and Pd(dppf)Cl₂ (609 mg, 0.84 mmol) in 1,4-Dioxane (40 mL) was stirred at 90° C. for 16 hours under N2 atmosphere. The mixture was concentrated. The residue was purified by column chromatography to afford title compound (2.30 g). ¹H NMR (400 MHz, CDCl₃) δ 8.26 (t, J=1.2 Hz, 1H), 8.01 (t, J=0.8 Hz, 1H), 7.77 (dd, J=1.2, 8.8 Hz, 1H), 7.43-7.36 (m, 1H), 4.88-4.82 (m, 1H), 1.59 (s, 3H), 1.58 (s, 3H), 1.36 (s, 12H). LCMS (ESI): m/z, 287.2 [M+H]⁺.

3. Synthesis of (1-Isopropyl-1H-indazol-5-yl) boronic acid. A mixture of 1-isopropyl-5-(4,4,5,5-tetramethyl-1,3,2-dioxaborolan-2-yl)-1H-indazole (2.20 g, 7.69 mmol) and NaIO₄ (3.29 g, 15.37 mmol) in THF (25 mL) and H₂O (5 mL) was stirred at 25° C. for 16 hours. The mixture was concentrated. The residue was purified by column chromatography to afford title compound (1.43 g). ¹H NMR (400 MHz, CDCl₃) (δ 8.78 (s, 1H), 8.25 (dd, J=1.2, 8.4 Hz, 1H), 8.19 (s, 1H), 7.57 (d, J=8.4 Hz, 1H), 4.94 (m, 1H), 1.66 (d, J=6.8 Hz, 6H). LCMS (ESI): 205.0, 206.0 [M+H]⁺. [001303]4. Synthesis of 2,4-Dichloro-7-(1-isopropyl-1H-indazol-5-yl)-5,5-dimethyl-5,7-dihydro-6H-pyrrolo[2,3-d]pyrimidin-6-one. A mixture of (1-isopropyl-1H-indazol-5-yl) boronic acid (879 mg, 4.31 mmol), 2,4-dichloro-5,5-dimethyl-5,7-dihydro-6H-pyrrolo[2,3-d]pyrimidin-6-one (500 mg, 2.15 mmol), Cu(OAc)₂ (470 mg, 2.59 mmol) and pyridine (511 mg, 6.46 mmol) in 1,2-dichloroethane (17 mL) was stirred at 60° C. for 16 hours under O₂. The reaction mixture was extracted with EtOAc. The organic layers were washed with brine, dried over Na₂SO₄, filtered and concentrated. The residue was purified by column chromatography to afford title compound (600 mg). ¹H NMR (400 MHz, CDCl₃) δ 8.08 (s, 1H), 7.78 (d, J=2.0 Hz, 1H), 7.57 (d, J=8.8 Hz, 1H), 7.35 (dd, J=2.0, 9.2 Hz, 1H), 4.88 (m, 1H), 1.66 (s, 6H), 1.63 (s, 3H), 1.61 (s, 3H). LCMS (ESI): m/z, 390.1 [M+H]⁺.

5. Synthesis of 2-(((tert-Butyldimethylsilyl)oxy)methyl)-4-chloro-7-(1-isopropyl-1H-indazol-5-yl)-5,5-dimethyl-5,7-dihydro-6H-pyrrolo[2,3-d]pyrimidin-6-one. A mixture of 2,4-dichloro-7-(1-isopropyl-1H-indazol-5-yl)-5,5-dimethyl-5,7-dihydro-6H-pyrrolo[2,3-d]pyrimidin-6-one (500 mg, 1.28 mmol), Pd (PPh₃)₄ (296 mg, 0.26 mmol) and Bu₃SnCH₂OTBS (837 mg, 1.92 mmol) in 1,4-Dioxane (8 mL) was stirred at 130° C. for 3 hours under N2. The mixture was concentrated. The residue was purified by column chromatography to afford title compound (385 mg). LCMS (ESI): m/z, 500.2 [M+H]⁺.

6. Synthesis of 2-(((tert-Butyldimethylsilyl)oxy)methyl)-7-(1-isopropyl-1H-indazol-5-yl)-5,5-dimethyl-4-(methylamino)-5,7-dihydro-6H-pyrrolo[2,3-d]pyrimidin-6-one. A mixture of 2-(((tert-butyldimethylsilyl)oxy)methyl)-4-chloro-7-(1-isopropyl-1H-indazol-5-yl)-5,5-dimethyl-5,7-dihydro-6H-pyrrolo[2,3-d]pyrimidin-6-one (360 mg, 60.0% purity, 0.46 mmol), methanamine hydrochloride (124 mg, 1.83 mmol) and K₂CO₃ (158 mg, 1.14 mmol) in DMA (7 mL) was stirred at 120° C. for 16 hours. The reaction mixture was extracted with EtOAc. The organic layers were washed with brine, dried over Na₂SO₄, filtered and concentrated to give title compound (260 mg crude). LCMS (ESI): m/z, 495.3 [M+H]⁺.

7. Synthesis of 2-(Hydroxymethyl)-7-(1-isopropyl-1H-indazol-5-yl)-5,5-dimethyl-4-(methylamino)-5,7-dihydro-6H-pyrrolo[2,3-d]pyrimidin-6-one. A mixture of 2-(((tert-butyldimethylsilyl)oxy)methyl)-7-(1-isopropyl-1H-indazol-5-yl)-5,5-dimethyl-4-(methylamino)-5,7-dihydro-6H-pyrrolo[2,3-d]pyrimidin-6-one (240 mg, 0.49 mmol), TBAF (381 mg, 1.46 mmol) in THF (5 mL) was stirred at 25° C. for 2 hours. The reaction mixture was extracted with EtOAc. The organic layers were washed with brine, dried over Na₂SO₄, filtered and concentrated. The residue was purified by column chromatography to afford title compound (72 mg). LCMS (ESI): m/z, 381.2 [M+H]⁺.

8. Synthesis of (7-(1-Isopropyl-1H-indazol-5-yl)-5,5-dimethyl-4-(methylamino)-6-oxo-6,7-dihydro-5H-pyrrolo[2,3-d]pyrimidin-2-yl)methyl 2-(trifluoromethyl)pyrrolidine-1-carbimidate. A mixture of 2-(hydroxymethyl)-7-(1-isopropyl-1H-indazol-5-yl)-5,5-dimethyl-4-(methylamino)-5,7-dihydro-6H-pyrrolo[2,3-d]pyrimidin-6-one (72 mg, 0.19 mmol), 2-(trifluoromethyl)pyrrolidine-1-carbonitrile (93 mg, 0.57 mmol), ZnCl₂ (103 mg, 0.76 mmol) in dioxane (1 mL) was stirred at 60° C. for 16 hours under N₂. The reaction mixture was filtered and concentrated under reduced pressure to give the residue. The crude product was purified by Prep-HPLC to afford title compound (34 mg). ¹H NMR (400 MHz, CDCl₃) δ 8.03 (s, 1H), 7.79 (d, J=2.0 Hz, 1H), 7.53 (d, J=9.2 Hz, 1H), 7.40 (dd, J=2.0, 8.8 Hz, 1H), 5.27-5.05 (m, 2H), 4.90-4.83 (m, 1H), 4.47-4.44 (m, 2H), 3.46 (m, 2H), 3.10 (d, J=4.8 Hz, 3H), 2.00-1.90 (m, 2H), 1.82-1.75 (m, 2H), 1.60 (d, J=6.8 Hz, 6H), 1.53 (s, 6H). LCMS (ESI): m/z, 545.2 [M+H]⁺.

### Example 242. Preparation of Compound E179

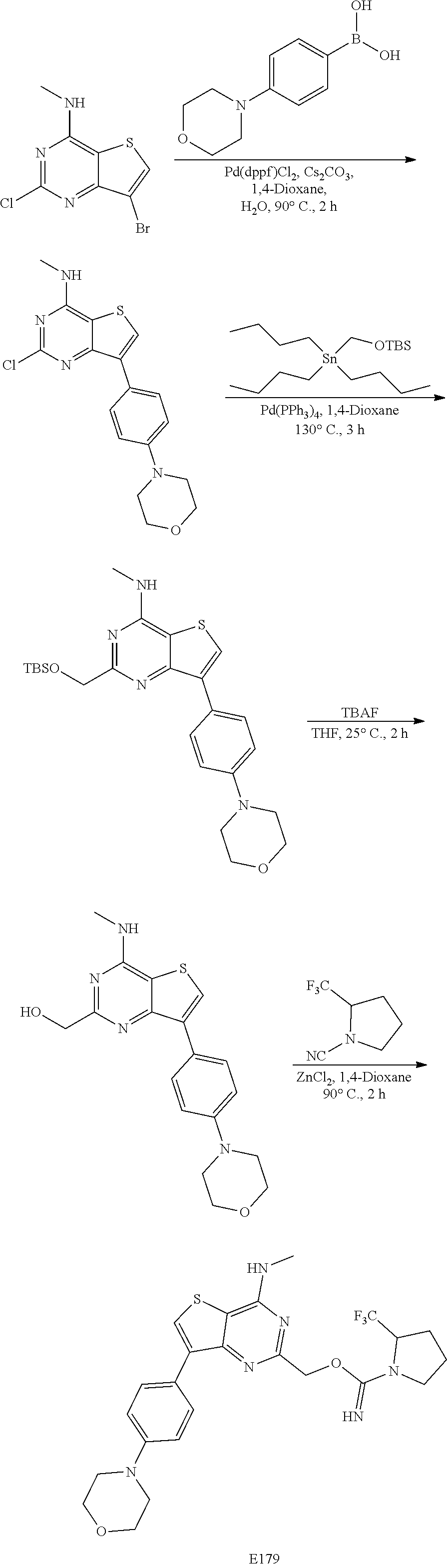

1. Synthesis of 2-Chloro-N-methyl-7-(4-morpholinophenyl)thieno[3,2-d]pyrimidin-4-amine. To a solution of 7-bromo-2-chloro-N-methylthieno[3,2-d]pyrimidin-4-amine (1.0 g, 3.59 mmol) and (4-morpholinophenyl)boronic acid (743 mg, 3.59 mmol) in H₂O (4 mL) and 1,4-Dioxane (20 mL) were added Pd(dppf)Cl₂ (263 mg, 0.36 mmol) and Cs₂CO₃ (2.34 g, 7.18 mmol). The mixture was stirred at 90° C. for 2 h under N₂. The solvent was removed to yield a residue which was purified by column chromatography to give title compound (480 mg). ¹H NMR (400 MHz, CDCl₃) δ 7.91-7.82 (m, 2H), 7.67 (s, 1H), 7.03-6.98 (m, 2H), 3.88 (dd, J=2.4, 4.0 Hz, 4H), 3.26-3.20 (m, 7H). LCMS (ESI): m/z, 361.0 [M+H]⁺.

2. Synthesis of 2-(((tert-Butyldimethylsilyl)oxy)methyl)-N-methyl-7-(4-morpholinophenyl)thieno[3,2-d]pyrimidin-4-amine. To a solution of 2-chloro-N-methyl-7-(4-morpholinophenyl)thieno[3,2-d]pyrimidin-4-amine (480 mg, 1.33 mmol) and tert-butyldimethyl((tributylstannyl)methoxy)silane (637 mg, 1.46 mmol) in 1,4-Dioxane (9 mL) was added Pd(PPh₃)₄ (154 mg, 0.13 mmol). The mixture was stirred at 130° C. for 3 h in microwave. The solvent was removed to yield a residue which was purified by column chromatography to give title compound (342 mg). LCMS (ESI): m/z, 471.3 [M+H]⁺.

3. Synthesis of (4-(Methylamino)-7-(4-morpholinophenyl)thieno[3,2-d]pyrimidin-2-yl)methanol. To a solution of 2-(((tert-butyldimethylsilyl)oxy)methyl)-N-methyl-7-(4-morpholinophenyl)thieno[3,2-d]pyrimidin-4-amine (342 mg, 0.73 mmol) in THF (5 mL) was added TBAF (2.91 mL, 1 M). The mixture was stirred at 25° C. for 2 h. The reaction was diluted with water, extracted with EtOAc. The combined organic phases were washed with brine, dried over anhydrous Na₂SO₄, filtered and concentrated. The residue was purified by column chromatography to give title compound (100 mg).

¹H NMR (400 MHz, CDCl₃) δ 7.87 (d, J=8.8 Hz, 2H), 7.66 (s, 1H), 7.01 (d, J=8.8 Hz, 2H), 4.99 (s, 1H), 4.76 (s, 2H), 4.15 (s, 1H), 3.97-3.82 (m, 4H), 3.23 (t, J=4.8 Hz, 7H). LCMS (ESI): m/z, 357.1 [M+H]⁺.

4. Synthesis of (4-(Methylamino)-7-(4-morpholinophenyl)thieno[3,2-d]pyrimidin-2-yl)methyl 2-(trifluoromethyl)pyrrolidine-1-carbimidate. To a solution of (4-(methylamino)-7-(4-morpholinophenyl)thieno[3,2-d]pyrimidin-2-yl)methanol (100 mg, 0.28 mmol) and 2-(trifluoromethyl)pyrrolidine-1-carbonitrile (138 mg, 0.84 mmol) in 1,4-Dioxane (5 mL) was added ZnCl₂ (153 mg, 1.12 mmol).The mixture was stirred at 90° C. for 2 h under N₂. The reaction was filtered and concentrated under reduce pressure to give a residue which was purified by Prep-HPLC to give title compound (22 mg). ¹H NMR (400 MHz, CDCl₃) δ 8.02-7.85 (m, 2H), 7.64 (s, 1H), 7.07-6.89 (m, 2H), 5.39 (d, J=12.0 Hz, 1H), 5.24 (d, J=16.0 Hz, 1H), 4.95 (s, 1H), 4.62 (m, 1H), 3.96-3.81 (m, 4H), 3.55 (m, 2H), 3.21 (m, 7H), 2.10 (s, 2H), 2.00-1.96 (m, 2H). LCMS (EST): m/z, 521.2 [M+H]⁺.

### Example 243. Preparation of Compound E1 83

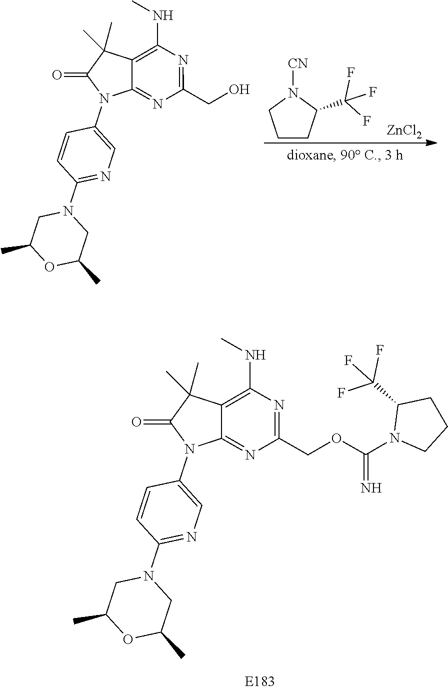

To a solution of 7-(6-((2S,6R)-2,6-dimethylmorpholino)pyridin-3-yl)-2-(hydroxymethyl)-5,5-dimethyl-4-(methylamino)-5,7-dihydro-6H-pyrrolo[2,3-d]pyrimidin-6-one (0.1 g, 0.24 mmol), (S)-2-(trifluoromethyl)pyrrolidine-1-carbonitrile (119 mg, 0.73 mmol) in Dioxane (3 mL) was added ZnCl₂ (99 mg, 0.73 mmol). The mixture was stirred at 90° C. for 3 h under N₂ atmosphere. The reaction was concentrated and purified by prep-HPLC to give title compound (31 mg). ¹H NMR (400 MHz, CDCl₃) δ 8.29 (m, 1H), 7.62-7.60 (m, ¹H), 6.69-6.67 (m, 1H), 5.22-5.20 (m, 1H), 5.06-5.04 (m, 1H), 4.48 (t, J=8.0 Hz, 1H), 4.41 (d, J=5.2 Hz, 1H), 4.10-4.08 (m, 2H), 3.73-3.72 (m, 2H), 3.51-3.40 (m, 2H), 3.08 (d, J=4.8 Hz, 3H), 2.57-2.55 (m, 2H), 2.09-2.03 (m, 2H), 1.95-1.93 (m, 2H), 1.48 (s, 6H), 1.27 (d, J=6.4 Hz, 6H). LCMS (ESI): m/z, 548.3 [M+H]⁺.

### Example 244. Preparation of Compound E184

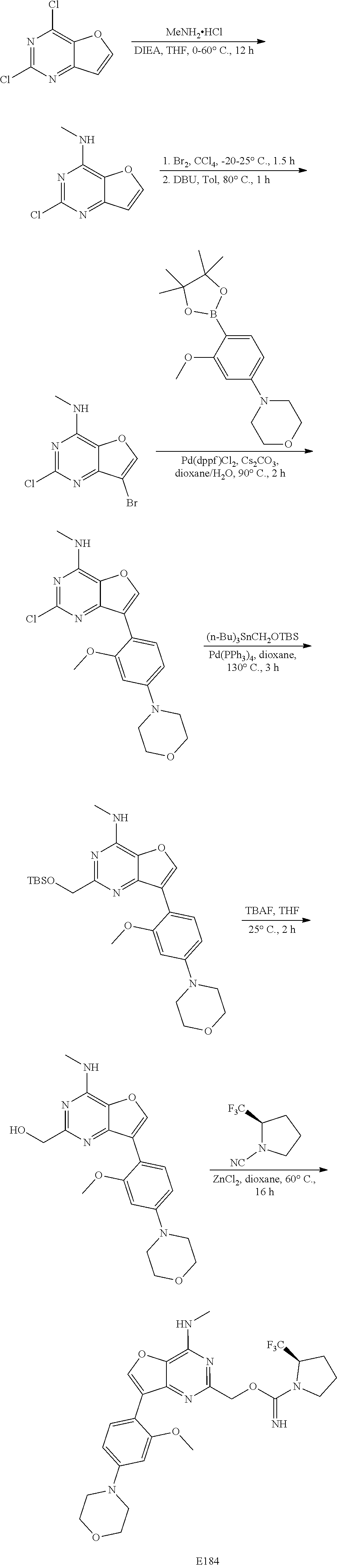

1. Synthesis of 2-chloro-N-methylfuro[3,2-d]pyrimidin-4-amine. To a mixture of 2,4-dichlorofuro[3,2-d]pyrimidine (900 mg, 4.76 mmol) and methanamine hydrochloride (804 mg, 11.90 mmol, 2.5 eq) in THF (10 mL) was added DIPEA (1.85 g, 2.36 mL, 14.29 mmol, 3 eq) at 0° C. The mixture was stirred at 60° C. for 12 hours under N₂. To the mixture was added H₂O and EtOAc, the aqueous phase was extracted with EtOAc. The combined organic layers were washed with brine and dried over Na₂SO₄, filtered and concentrated. The residue was purified by column chromatography to give title compound (812 mg). LCMS (ESI): m/z, 184.1, 186.1 [M+H]⁺.

2. Synthesis of 7-Bromo-2-chloro-N-methylfuro[3,2-d]pyrimidin-4-amine. To a mixture of 2-chloro-N-methylfuro[3,2-d]pyrimidin-4-amine (812 mg, 4.42 mmol) in CCl₄ (10 mL) was added Br₂ (10.60 g, 66.34 mmol) at −20° C., the mixture was stirred at 25° C. for 1.5 h under N₂. The mixture was poured into a mixture of water (100 mL) and Na₂SO₃ (10 g). The resulting mixture was extracted with EtOAc. The organic phase was dried over MgSO₄, filtered, and the solvent was evaporated. PhMe (10 mL) and DBU (2.02 g, 1.98 mL, 13.27 mmol, 3 eq) were added to the residue and the mixture was stirred under N₂ at 80° C. for 1 h. To the mixture was added H₂O and EtOAc, the aqueous phase was extracted with EtOAc. The combined organic layers were washed with brine and dried over Na₂SO₄, filtered and concentrated. The residue was purified by column chromatography to give title compound (460 mg). ¹H NMR (400 MHz, DMSO-dₖ) b 8.55 (s, 2H), 2.94 (d, J=4.4 Hz, 3H). LCMS (ESI): m/z, 261.9, 263.9 [M+H]⁺.

3. Synthesis of 2-Chloro-7-(2-methoxy-4-morpholinophenyl)-N-methylfuro[3,2-d]pyrimidin-4-amine. To a solution of 7-bromo-2-chloro-N-methylfuro[3,2-d]pyrimidin-4-amine (630 mg, 2.40 mmol) and 4-[3-(methyloxy)-4-(4,4,5,5-tetramethyl-1,3,2-dioxaborolan-2-yl)phenyl]morpholine (1.53 g, 4.80 mmol) in 1,4-dioxane (16 mL) and H₂O (2 mL) were added Pd(dppf)Cl₂ (175 mg, 0.24 mmol) and Cs₂CO₃ (2.35 g, 7.20 mmol). The mixture was stirred at 90° C. for 2 h under N₂. To the mixture was added H₂O and EtOAc, the aqueous phase was extracted with EtOAc. The combined organic layers were washed with brine and dried over Na₂SO₄, filtered and concentrated. The residue was purified by column chromatography to give title compound (1.21 g crude). ¹H NMR (400 MHz, DMSO-de) δ 8.55 (s, 1H), 8.44 (d, J=8.4 Hz, 1H), 8.32 (s, 1H), 6.71-6.62 (m, 2H), 3.94 (s, 3H), 3.80-3.73 (m, 4H), 3.21 (dd, J=4.0, 6.0 Hz, 4H), 2.96 (s, 3H). LCMS (ESI): m/z, 375.1, 377.1 [M+H]⁺.

4. Synthesis of 2-(((tert-Butyldimethylsilyl)oxy)methyl)-7-(2-methoxy-4-morpholinophenyl)-N-methylfuro[3,2-d]pyrimidin-4-amine. To a solution of 2-chloro-7-[2-methoxy-4-(morpholin-4-yl)phenyl]-N-methylfuro[3,2-d]pyrimidin-4-amine (1.10 g, 2.93 mmol) in 1,4-Dioxane (10 mL) were added Bu₃SnCH₂OTBS (1.28 g, 2.93 mmol) and Pd(PPh₃)₄ (339 mg, 0.29 mol). The mixture was stirred at 130° C. for 3 h in microwave under N₂. The mixture was directly concentrated under reduced pressure. The residue was purified by column chromatography to give title compound (970 mg). LCMS (ESI): m/z, 485.3 [M+H]⁺.

5. Synthesis of (7-(2-Methoxy-4-morpholinophenyl)-4-(methylamino)furo[3,2-d]pyrimidin-2-yl)methanol. To a solution of 2-{[(tert-butyldimethylsilyl)oxy]methyl}-7-[2-methoxy-4-(morpholin-4-yl)phenyl]-N-methylfuro[3,2-d]pyrimidin-4-amine (870 mg, 1.80 mmol) in THF (8 mL) was added TBAF (5.39 mL, 1 M in THF). The mixture was stirred at 25° C. for 2 h. To the mixture was added H₂O and EtOAc, the aqueous phase was extracted with EtOAc. The combined organic layers were washed with brine and dried over Na₂SO₄, filtered and concentrated. The residue was purified by column chromatography to give title compound (396 mg). LCMS (ESI): m/z, 371.1 [M+H]⁺.

6. Synthesis of (7-(2-Methoxy-4-morpholinophenyl)-4-(methylamino)furo[3,2-d]pyrimidin-2-yl)methyl (R)-2-(trifluoromethyl)pyrrolidine-1-carbimidate. To a solution of {7-[2-methoxy-4-(morpholin-4-yl)phenyl]-4-(methylamino)furo[3,2-d]pyrimidin-2-yl}methanol (100 mg, 0.27 mmol) and 2-(trifluoromethyl)pyrrolidine-1-carbonitrile (133 mg, 0.81 mmol) in 1,4-dioxane (4 mL) was added ZnCl₂ (147 mg, 1.08 mmol). The mixture was stirred at 60° C. for 16 h under N₂. The mixture was filtered. The filtrate was purified by Prep-HPLC to give title compound (58.70 mg). ¹H NMR (400 MHz, Chloroform-d) δ 8.75 (d, J=8.4 Hz, 1H), 8.29 (s, 1H), 6.64 (dd, J=2.4, 8.4 Hz, 1H), 6.53 (d, J=2.4 Hz, 1H), 5.41-5.04 (m, 3H), 4.68-4.56 (m, 1H), 3.93 (s, 3H), 3.91-3.87 (m, 4H), 3.53 (t, J=6.4 Hz, 2H), 3.26-3.14 (m, 7H), 2.16-2.00 (m, 4H). LCMS (ESI): m/z, 535.2 [M+H]⁺.

### Example 245. Preparation of Compound E186

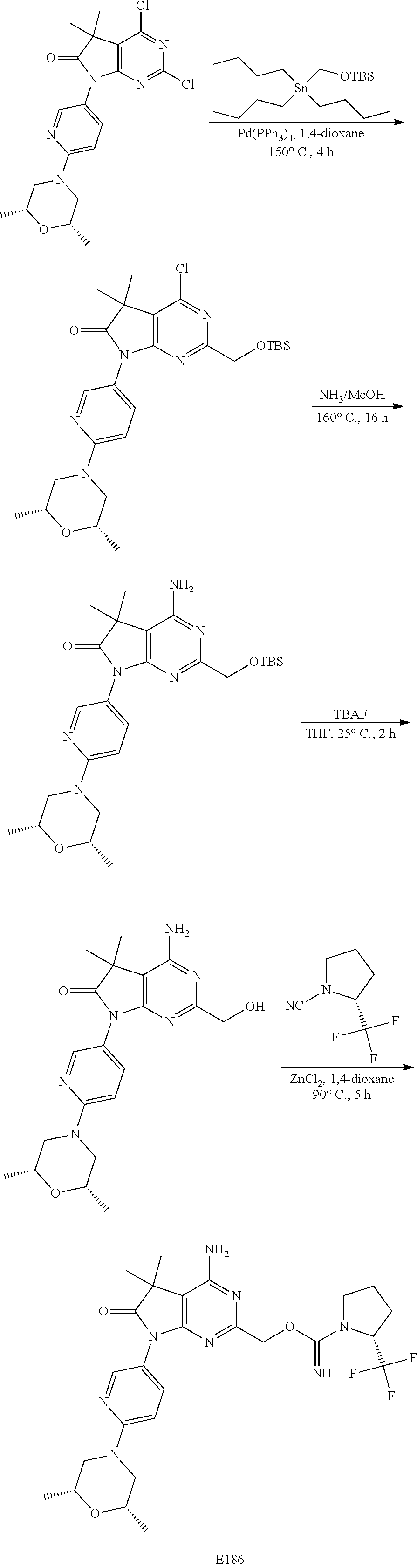

1. Synthesis of 2-(((tert-Butyldimethylsilyl)oxy)methyl)-4-chloro-7-(6-((2S,6R)-2,6-dimethylmorpholino)pyridin-3-yl)-5,5-dimethyl-5,7-dihydro-6H-pyrrolo[2,3-d]pyrimidin-6-one. To a mixture of 2,4-dichloro-7-(6-((2S,6R)-2,6-dimethylmorpholino)pyridin-3-yl)-5,5-dimethyl-5,7-dihydro-6H-pyrrolo[2,3-d]pyrimidin-6-one (1.50 g, 3.55 mmol) in 1,4-Dioxane (20 mL) was added Bu₃SnCH₂OTBS (1.86 g, 4.26 mmol) and Pd(PPh₃)₄ (410 mg, 0.36 mmol). The mixture was purged by N₂ and stirred at 150° C. for 4 hours under microwave. The reaction mixture was concentrated. The crude was purified by column chromatography to give title compound (1.89 g).

2. Synthesis of 4-amino-2-(((tert-butyldimethylsilyl)oxy)methyl)-7-(6-((2S,6R)-2,6-dimethylmorpholino)pyridin-3-yl)-5,5-dimethyl-5,7-dihydro-6H-pyrrolo[2,3-d]pyrimidin-6-one. A solution of 1.2-(((tert-butyldimethylsilyl)oxy)methyl)-4-chloro-7-(6-((2S,6R)-2,6-dimethylmorpholino)pyridin-3-yl)-5,5-dimethyl-5,7-dihydro-6H-pyrrolo[2,3-d]pyrimidin-6-one (300 mg, 0.56 mmol) in NH₃ (9 g, 10 mL, 528.48 mmol) in MeOH was stirred at 160° C. for 16 hours in autoclave. The mixture was concentrated. The mixture was concentrated to yield title compound (100 mg). LCMS (ESI): m/z, 513.4 [M+H]⁺.

3. Synthesis of 4-amino-7-(6-((2S,6R)-2,6-dimethylmorpholino)pyridin-3-yl)-2-(hydroxymethyl)-5,5-dimethyl-5,7-dihydro-6H-pyrrolo[2,3-d]pyrimidin-6-one. To a solution of 4-amino-2-(((tert-butyldimethylsilyl)oxy)methyl)-7-(6-((2S,6R)-2,6-dimethylmorpholino)pyridin-3-yl)-5,5-dimethyl-5,7-dihydro-6H-pyrrolo[2,3-d]pyrimidin-6-one (100 mg, 0.19 mmol) in THF (2 mL) was added TBAF (1 M, 1.65 mL) and the mixture was stirred at 25° C. for 2 h. The reaction was diluted with water, extracted with EtOAc. The combined organic phases were washed with brine, dried over anhydrous Na₂SO₄, filtered and concentrated was purified by column chromatography to give title compound (50 mg).

4. Synthesis of (4-amino-7-(6-((2S,6R)-2,6-dimethylmorpholino)pyridin-3-yl)-5,5-dimethyl-6-oxo-6,7-dihydro-5H-pyrrolo[2,3-d]pyrimidin-2-yl)methyl (R)-2-(trifluoromethyl)pyrrolidine-1-carbimidate. To a solution of 4-amino-7-(6-((2S,6R)-2,6-dimethylmorpholino)pyridin-3-yl)-2-(hydroxymethyl)-5,5-dimethyl-5,7-dihydro-6H-pyrrolo[2,3-d]pyrimidin-6-one (100 mg, 0.25 mmol) and (2R)-2-(trifluoromethyl)pyrrolidine-1-carbonitrile (123 mg, 0.75 mmol) in 1,4-dioxane (20 mL) was added ZnCl₂ (85 mg, 0.63 mmol). The mixture was stirred at 60° C. for 16 hours under N₂. The mixture was stirred at 90° C. for 2 hours under N₂. The mixture was filtered. The filtrate was purified by Prep-HPLC to yield title compound (110 mg). ¹H NMR (400 MHz, Chloroform-l) δ 8.30 (d, J=2.8 Hz, 1H), 7.60 (dd, J=2.8, 9.2 Hz, 1H), 6.67 (d, J=9.2 Hz, 1H), 5.18 (d, J=14.4 Hz, 1H), 5.02 (d, J=14.6 Hz, 1H), 4.80 (s, 2H), 4.44 (p, J=7.8 Hz, 1H), 4.09 (dq, J=1.6, 11.2 Hz, 2H), 3.71-3.69 (m, 2H), 3.45 (dt, J=8.4, 19.2 Hz, 2H), 2.55 (dd, J=10.8, 12.8 Hz, 2H), 2.13-2.00 (m, 2H), 1.92 (t, J=8.0 Hz, 2H), 1.52 (s, 6H), 1.28 (d, J=6.4 Hz, 6H). LCMS (ESI): m/z, 563.3 [M+H]⁺.

### Example 246. Preparation of Compound E187

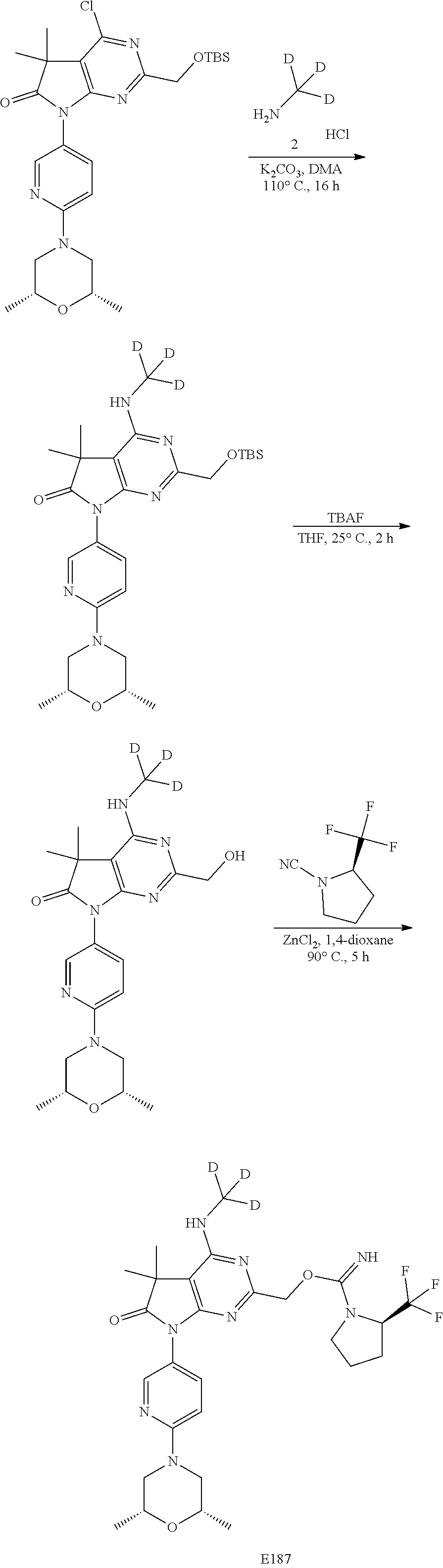

1. Synthesis of 2-(((tert-Butyldimethylsilyl)oxy)methyl)-7-(6-((2S,6R)-2,6-dimethylmorpholino)pyridin-3-yl)-5,5-dimethyl-4-((methyl-d3)amino)-5,7-dihydro-6H-pyrrolo[2,3-d]pyrimidin-6-one. A solution of 2-(((tert-butyldimethylsilyl)oxy)methyl)-4-chloro-7-(6-((2S,6R)-2,6-dimethylmorpholino)pyridin-3-yl)-5,5-dimethyl-5,7-dihydro-6H-pyrrolo[2,3-d]pyrimidin-6-one (200 mg, 0.38 mmol), methylamine-d3 hydrochloride (265 mg, 3.76 mmol) and K₂CO₃ (155 mg, 1.13 mmol) in DMA (8 mL) was stirred at 110° C. for 16 hours in sealed tube. After filtration, the filtrate layer was added water and extracted with EA. The combined organic layers were washed with brine and dried over Na₂SO₄, filtered. The mixture was concentrated to yield title compound (199 mg). LCMS (ESI): m/z, 530.4 [M+H]⁺.

2. Synthesis of 7-(6-((2S,6R)-2,6-Dimethylmorpholino)pyridin-3-yl)-2-(hydroxymethyl)-5,5-dimethyl-4-((methyl-d3)amino)-5,7-dihydro-6H-pyrrolo[2,3-d]pyrimidin-6-one. To a solution of 2-(((tert-butyldimethylsilyl)oxy)methyl)-7-(6-((2S,6R)-2,6-dimethylmorpholino)pyridin-3-yl)-5,5-dimethyl-4-((methyl-d3)amino)-5,7-dihydro-6H-pyrrolo[2,3-d]pyrimidin-6-one (199 mg, 0.37 mmol) in THF (10 mL) was added TBAF (1.13 mL, 1 M in THF). The mixture was stirred at 25° C. for 2 h. Water was added and extracted with EtOAc. The combined organic phases were washed with brine, dried over anhydrous Na₂SO₄, filtered and concentrated. The residue was purified by column chromatography to give title compound (90 mg). LCMS (ESI): m/z, 416.2 [M+H]⁺.

3. Synthesis of (7-(6-((2S,6R)-2,6-Dimethylmorpholino)pyridin-3-yl)-5,5-dimethyl-4-((methyl-d3)amino)-6-oxo-6,7-dihydro-5H-pyrrolo[2,3-d]pyrimidin-2-yl)methyl (R)-2-(trifluoromethyl)pyrrolidine-1-carbimidate. To a solution of (2R)-2-(trifluoromethyl)pyrrolidine-1-carbonitrile (106 mg, 0.65 mmol) and 7-(6-((2S,6R)-2,6-dimethylmorpholino)pyridin-3-yl)-2-(hydroxymethyl)-5,5-dimethyl-4-((methyl-d₃)amino)-5,7-dihydro-6H-pyrrolo[2,3-d]pyrimidin-6-one (90 mg, 0.22 mmol) in 1,4-Dioxane (10 mL) was added ZnCl₂ (74 mg, 0.54 mmol). The mixture was stirred at 60° C. for 16 hours under N₂. The mixture was filtered. The filtrate was purified by Prep-HPLC to yield title compound (38 mg). ¹H NMR (400 MHz, DMSO-d₆) δ 8.10 (d, J=2.4 Hz, 1H), 7.56 (dd, J=2.8, 9.2 Hz, 1H), 6.93 (d, J=9.2 Hz, 1H), 6.69 (s, 1H), 5.76 (s, 1H), 4.95 (t, J=18.8 Hz, 2H), 4.55 (s, 1H), 4.18 (d, J=12.0 Hz, 2H), 3.61 (ddd, J=9.6, 6.0, 2.4 Hz, 2H), 3.33-3.30 (m, 2H), 2.43 (dd, J=10.4, 12.8 Hz, 2H), 1.87 (d, J=32.8 Hz, 4H), 1.42 (s, 6H), 1.17 (d, J=6.0 Hz, 6H). LCMS (ESI): m/z, 580.4 [M+H]⁺.

### Example 247. Preparation of Compound E188

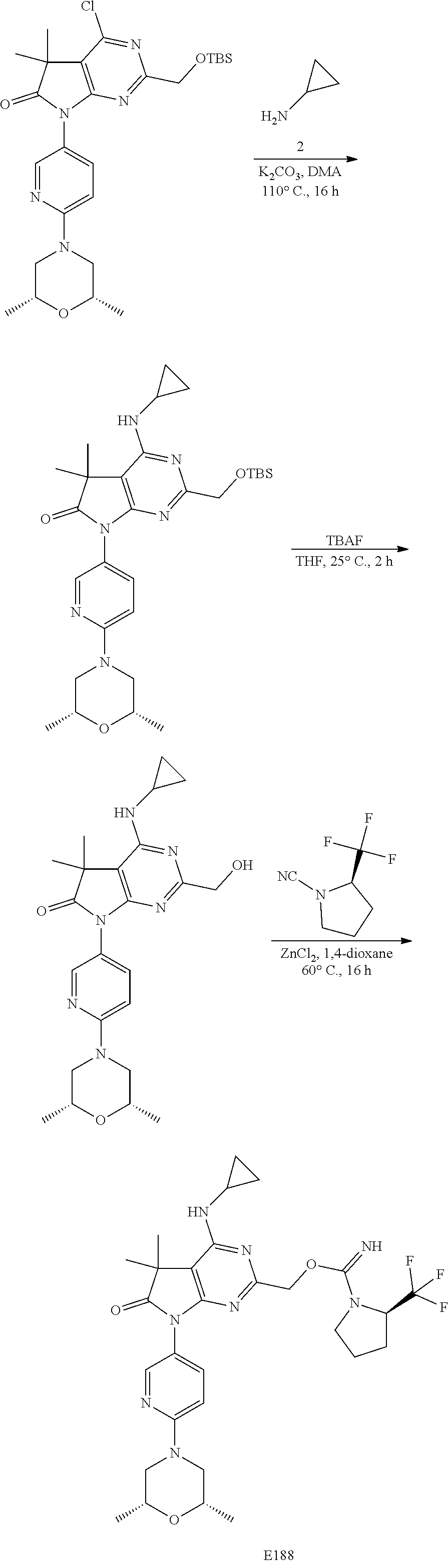

1. Synthesis of 2-(((tert-Butyldimethylsilyl)oxy)methyl)-4-(cyclopropylamino)-7-(6-((2S,6R)-2,6-dimethylmorpholino)pyridin-3-yl)-5,5-dimethyl-5,7-dihydro-6H-pyrrolo[2,3-d]pyrimidin-6-one. A solution of 2-(((tert-butyldimethylsilyl)oxy)methyl)-4-chloro-7-(6-((2S,6R)-2,6-dimethylmorpholino)pyridin-3-yl)-5,5-dimethyl-5,7-dihydro-6H-pyrrolo[2,3-d]pyrimidin-6-one (250 mg, 0.47 mmol), cyclopropylamine (268 mg, 4.70 mmol) and K₂CO₃ (194 mg, 1.41 mmol) in DMA (8 mL) was stirred at 110° C. for 16 hours in sealed tube. After filtration, the filtrate layer was extracted with EA. The combined organic layers were washed with brine and dried over Na₂SO₄, filtered. The mixture was concentrated to yield title compound (200 mg crude). LCMS (ESI): m/z, 553.4 [M+H]⁺.

2. Synthesis of 4-(Cyclopropylamino)-7-(6-((2S,6R)-2,6-dimethylmorpholino)pyridin-3-yl)-2-(hydroxymethyl)-5,5-dimethyl-5,7-dihydro-6H-pyrrolo[2,3-d]pyrimidin-6-one. To a solution of 2-(((tert-butyldimethylsilyl)oxy)methyl)-4-(cyclopropylamino)-7-(6-((2S,6R)-2,6-dimethylmorpholino)pyridin-3-yl)-5,5-dimethyl-5,7-dihydro-6H-pyrrolo[2,3-d]pyrimidin-6-one (210 mg, 0.38 mmol) in THF (10 mL) was added TBAF (1.14 mL, 1 M in THF). The mixture was stirred at 25° C. for 2 h. Water was added and extracted with EtOAc. The combined organic phases were washed with brine, dried over anhydrous Na₂SO₄, filtered and concentrated. The residue was purified by column chromatography to give title compound (70 mg). LCMS (ESI): m/z, 439.3 [M+H]⁺.

3. Synthesis of (4-(Cyclopropylamino)-7-(6-((2S,6R)-2,6-dimethylmorpholino)pyridin-3-yl)-5,5-dimethyl-6-oxo-6,7-dihydro-5H-pyrrolo[2,3-d]pyrimidin-2-yl)methyl (R)-2-(trifluoromethyl)pyrrolidine-1-carbimidate. To a solution of 4-(cyclopropylamino)-7-(6-((2S,6R)-2,6-dimethylmorpholino)pyridin-3-yl)-2-(hydroxymethyl)-5,5-dimethyl-5,7-dihydro-6H-pyrrolo[2,3-d]pyrimidin-6-one (70 mg, 0.16 mmol) and (2R)-2-(trifluoromethyl)pyrrolidine-1-carbonitrile (78 mg, 0.48 mmol) in 1,4-dioxane (10 mL) was added ZnCl₂ (54 mg, 0.40 mmol). The mixture was stirred at 60° C. for 16 hours under N₂. The mixture was filtered. The filtrate was purified by Prep-HPLC to yield title compound (39 mg). ¹H NMR (400 MHz, Chloroform-c) δ 8.27 (dd, J=0.8, 2.8 Hz, 1H), 7.57 (dd, J=2.8, 9.2 Hz, 1H), 6.70 (d, J=9.2 Hz, 1H), 5.28 (d, J=14.0 Hz, 1H), 5.13 (d, J=14.0 Hz, 1H), 4.73 (s, 1H), 4.62 (s, 1H), 4.09 (d, J=12.4 Hz, 2H), 3.72 (ddd, J=2.4, 6.4, 10.4 2H), 3.69-3.57 (m, 2H), 2.88 (dt, J=3.2, 6.0 Hz, 1H), 2.56 (dd, J=10.4, 12.8 Hz, 2H), 2.14 (d, J=9.6 Hz, 2H), 2.01 (s, 2H), 1.68-1.63 (m, 3H), 1.50-1.45 (m, 6H), 1.28 (d, J=6.4 Hz, 6H), 0.94 (d, J=6.4 Hz, 2H), 0.62-0.49 (m, 2H). LCMS (ESI): m/z, 603.3 [M+H]⁺.

### Example 248. Preparation of Compound E189

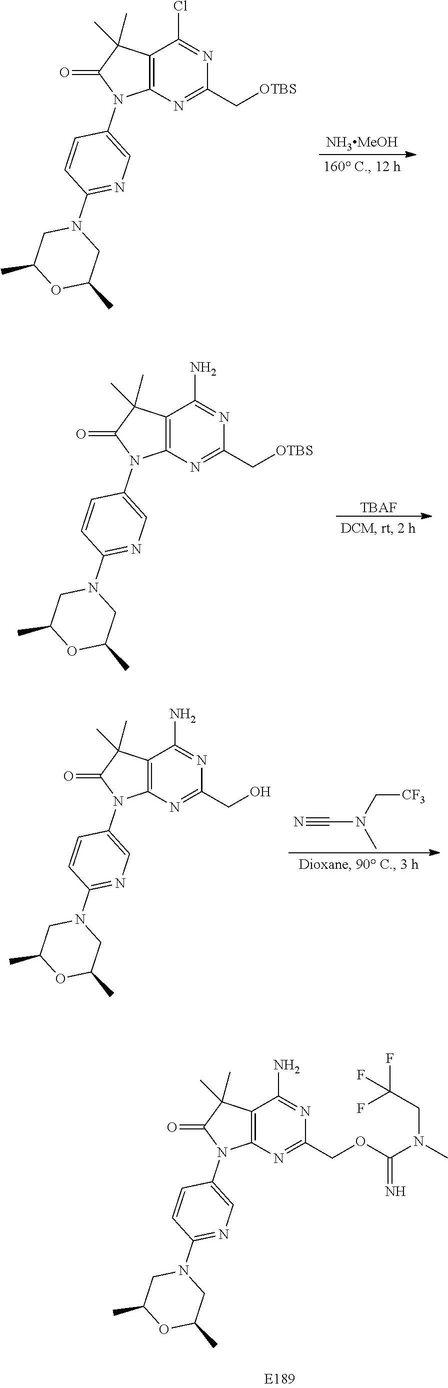

1. Synthesis of 4-amino-2-(((tert-butyldimethylsilyl)oxy)methyl)-7-(6-((2R,6S)-2,6-dimethylmorpholino)pyridin-3-yl)-5,5-dimethyl-5,7-dihydro-6H-pyrrolo[2,3-d]pyrimidin-6-one. A solution of 2-(((tert-butyldimethylsilyl)oxy)methyl)-4-chloro-7-(6-((2R,6S)-2,6-dimethylmorpholino)pyridin-3-yl)-5,5-dimethyl-5,7-dihydro-6H-pyrrolo[2,3-d]pyrimidin-6-one (1.90 g, 3.57 mmol) in NH₃ (30 mL, 7 M in MeOH) was stirred at 160° C. for 12 hours in a sealed tube. The reaction was concentrated to give title compound (2 g crude). LCMS (ESI): m/z, 513.3 [M+H]⁺.

2. Synthesis of 4-amino-7-(6-((2R,6S)-2,6-dimethylmorpholino)pyridin-3-yl)-2-(hydroxymethyl)-5,5-dimethyl-5,7-dihydro-6H-pyrrolo[2,3-d]pyrimidin-6-one. To a solution of 4-amino-2-(((tert-butyldimethylsilyl)oxy)methyl)-7-(6-((2R,6S)-2,6-dimethylmorpholino)pyridin-3-yl)-5,5-dimethyl-5,7-dihydro-6H-pyrrolo[2,3-d]pyrimidin-6-one (2 g, 3.90 mmol) in DCM (20 mL) was added TBAF (11.7 mL, 1 M in THF). The solution was stirred at 25° C. for 2 hours. The mixture was concentrated and purified by column chromatography to give title compound (850 mg). LCMS (ESI): m/z, 399.3 [M+H]⁺.

3. Synthesis of (4-amino-7-(6-((2R,6S)-2,6-dimethylmorpholino)pyridin-3-yl)-5,5-dimethyl-6-oxo-6,7-dihydro-5H-pyrrolo[2,3-d]pyrimidin-2-yl)methyl methyl(2,2,2-trifluoroethyl)carbamimidate. To a solution of N-methyl-N-(2,2,2-trifluoroethyl)cyanamide (104 mg, 0.75 mmol), 4-amino-7-(6-((2R,6S)-2,6-dimethylmorpholino)pyridin-3-yl)-2-(hydroxymethyl)-5,5-dimethyl-5,7-dihydro-6H-pyrrolo[2,3-d]pyrimidin-6-one (100 mg, 0.25 mmol) in 1,4-dioxane (3 mL) was added ZnCl₂ (103 mg, 0.75 mmol) at 25° C. The reaction was stirred at 90° C. for 3 hours. The mixture was concentrated and purified by Prep-HPLC to give title compound (92 mg). ¹H NMR (400 MHz, CDCl₃) δ 8.27 (s, 1H), 7.56-7.59 (m, 1H), 6.67-6.69 (m, 1H), 5.08 (s, 2H), 4.87 (s, 2H), 4.12-4.06 (m, 2H), 3.84-3.93 (m, 2H), 3.68-3.76 (m, 2H), 2.99 (s, 3H), 2.53-2.59 (m, 2H), 1.52 (s, 6H), 1.28 (d, J=6.4 Hz, 6H). LCMS (ESI): m/z, 537.3 [M+H]⁺.

### Example 249. Preparation of Compound E190

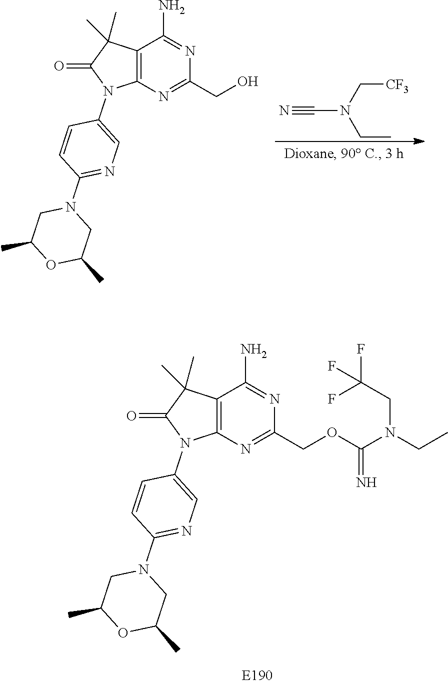

To a solution of N-ethyl-N-(2,2,2-trifluoroethyl)cyanamide (115 mg, 0.75 mmol), 4-amino-7-(6-((2R,6S)-2,6-dimethylmorpholino)pyridin-3-yl)-2-(hydroxymethyl)-5,5-dimethyl-5,7-dihydro-6H-pyrrolo[2,3-d]pyrimidin-6-one (100 mg, 0.25 mmol) in 1,4-Dioxane (3 mL) was added ZnCl₂ (103 mg, 0.75 mmol) at 25° C. The reaction was stirred at 90° C. for 3 hours. The mixture was concentrated and purified by prep-HPLC to give title compound (100 mg). ¹H NMR (400 MHz, CDCl₃) δ 8.26-8.27 (m, 1H), 7.55-7.58 (m, 1H), 6.66-6.69 (m, 1H), 5.06 (s, 2H), 4.93-4.78 (m, 2H), 4.12-4.06 (m, 2H), 3.83-3.90 (m, 2H), 3.67-3.75 (m, 2H), 3.33-3.39 (m, 2H), 2.53-2.59 (m, 2H), 1.52 (s, 6H), 1.28 (d, J=6.4 Hz, 6H), 1.11 (t, J=7.2 Hz, 3H). LCMS (ESI): m/z, 551.4 [M+H]⁺.

### Example 250. Preparation of Compound E178

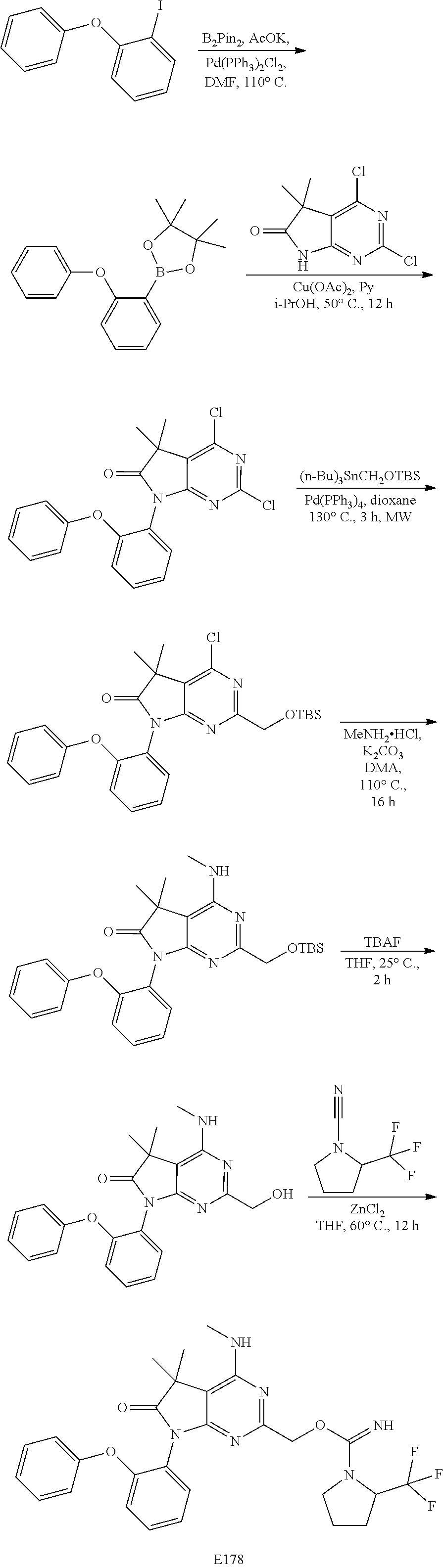

Compound E178 was synthesized as compound E29. LCMS (ESI): m/z, 551.4 [M+H]⁺.

### Example 251. Preparation of Compound E180 and E181

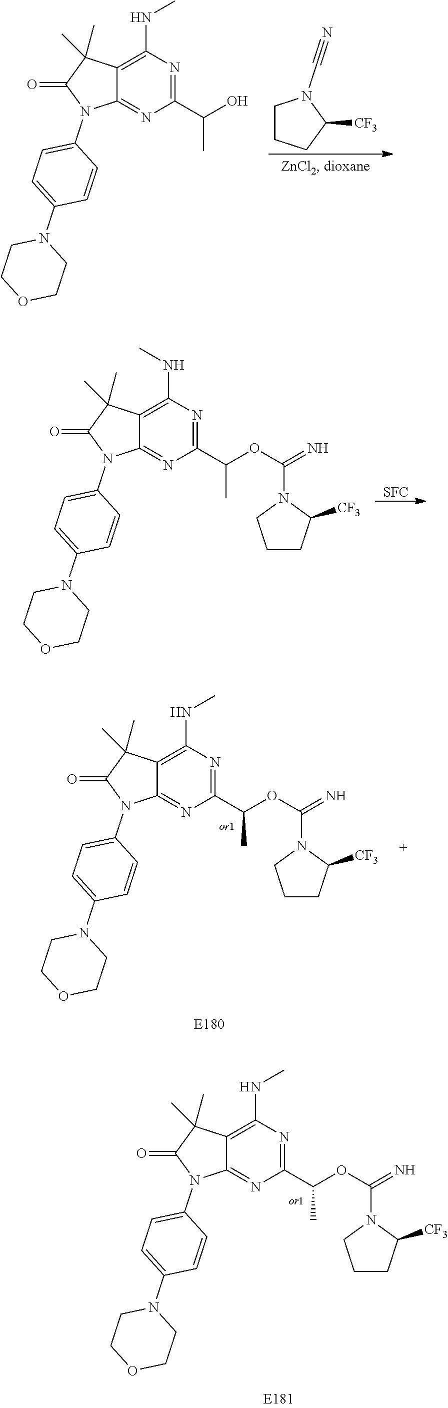

Compound E180 and E181 was synthesized as compound E152. LCMS (ESI): m/z, 562.3 [M+H]⁺; LCMS (ESI): m/z, 562.3 [M+H]⁺.

### Example 252. Preparation of Compound E182

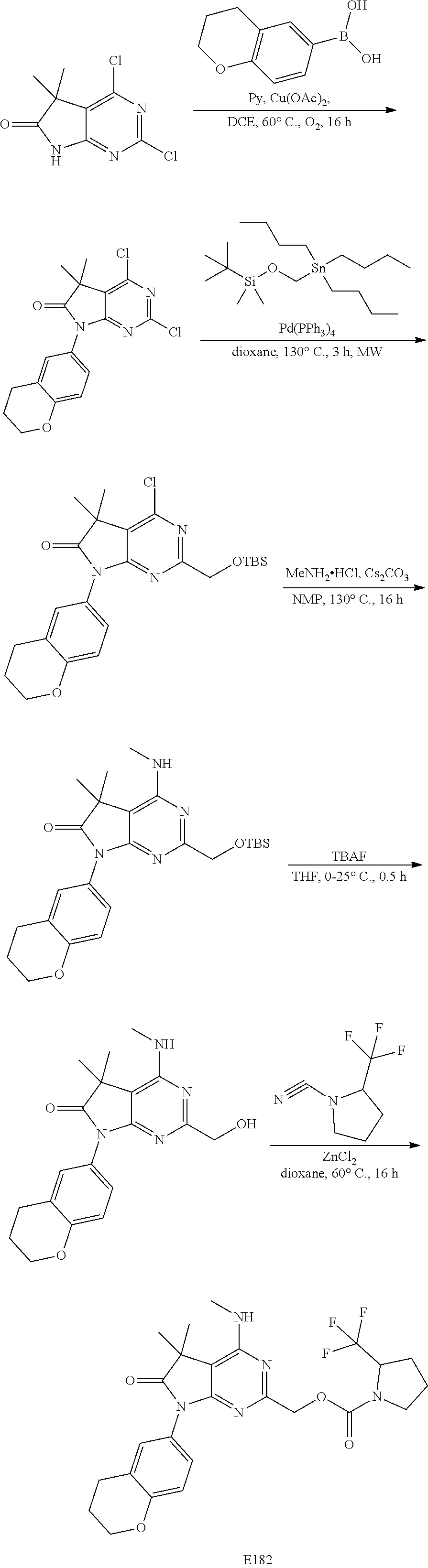

Compound E182 was synthesized as compound E68. LCMS (EST): m/z, 519.3 [M+H]⁺.

### Example 253. Preparation of Compound E185

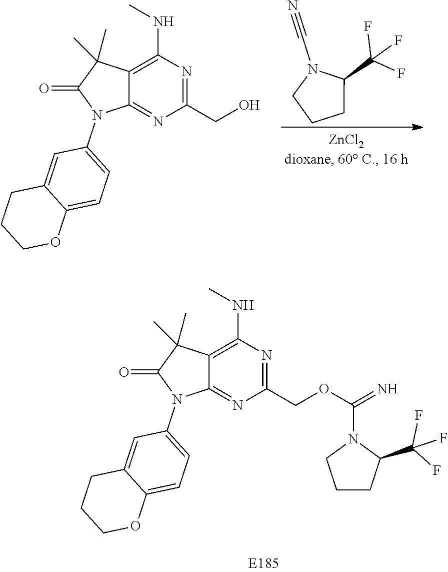 [SMILES: CNc1nc(CO)nc2c1C(C)(C)C(=O)N2c1ccc2c(c1)CCCO2 ; N#CN1CCC[C@@H]1C(F)(F)F ; CNc1nc(COC(=N)N2CCC[C@@H]2C(F)(F)F)nc2c1C(C)(C)C(=O)N2c1ccc2c(c1)CCCO2]

Compound E185 was synthesized as compound E182. LCMS (ESI): m/z, 519.3 [M+H]⁺.

### Example 254. Preparation of Compound E191.

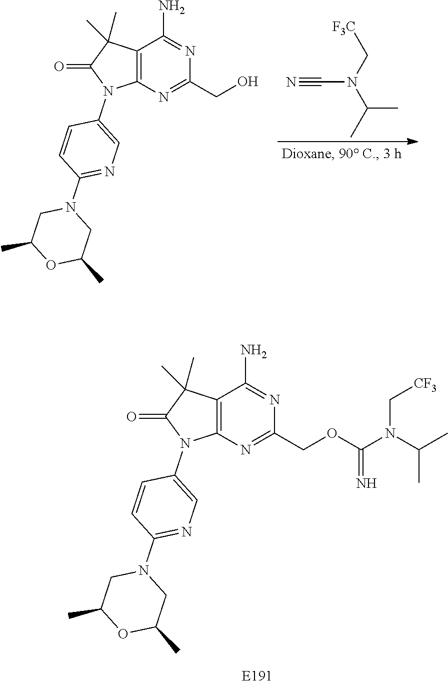 [SMILES: C[C@@H]1CN(c2ccc(N3C(=O)C(C)(C)c4c(N)nc(CO)nc43)cn2)C[C@H](C)O1 ; CC(C)N(C#N)CC(F)(F)F ; CC(C)N(CC(F)(F)F)C(=N)OCc1nc(N)c2c(n1)N(c1ccc(N3C[C@@H](C)O[C@@H](C)C3)nc1)C(=O)C2(C)C]

To a solution of N-isopropyl-N-(2,2,2-trifluoroethyl) cyanamide (125 mg, 0.75 mmol), 4-amino-7-(6-((2R,6S)-2,6-dimethylmorpholino) pyridin-3-yl)-2-(hydroxymethyl)-5,5-dimethyl-5,7-dihydro-6H-pyrrolo[2,3-d]pyrimidin-6-one (100 mg, 0.25 mmol) in 1,4-Dioxane (3 mL) was added ZnCl₂ (103 mg, 0.75 mmol) at 25° C. The reaction was stirred at 90° C. for 3 hours. The mixture was concentrated and purified by prep-HPLC to afford title compound. ¹H NMR (400 MHz, CDCl₃) (δ 8.26-8.27 (m, 1H), 7.55-7.57 (m, 1H), 6.66-6.69 (m, 1H), 5.05 (s, 2H), 4.86 (s, 2H), 4.14-4.04 (m, 3H), 3.80-3.86 (m, 2H), 3.67-3.75 (m, 2H), 2.53-2.59 (m, 2H), 1.52 (s, 6H), 1.28 (d, J=6.4 Hz, 6H), 1.16 (d, J=6.8 Hz, 6H). LCMS (ESI): m/z, 551.4 [M+H]⁺.

### Example 255. Preparation of Compound E192

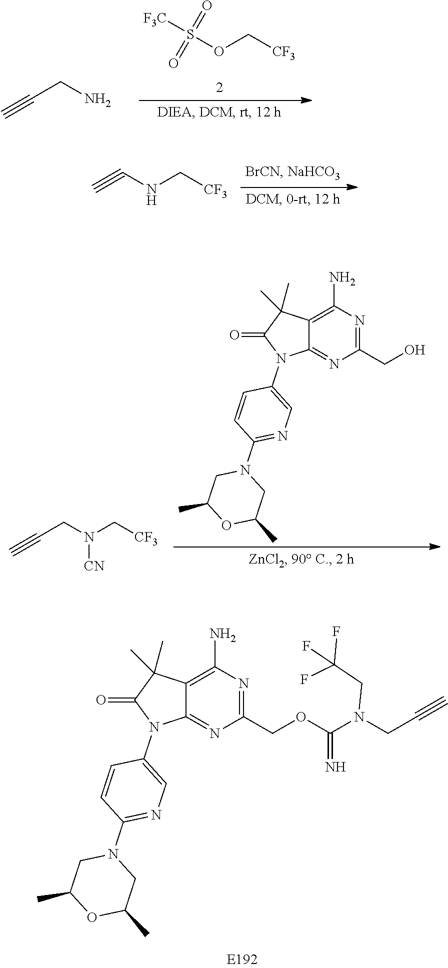

1. Synthesis of compound N-(2,2,2-trifluoroethyl)prop-2-yn-1-amine. A solution of 2,2,2-trifluoroethyl trifluoromethanesulfonate (8.43 g, 36.31 mmol), Propargylamine (1 g, 18.16 mmol) and DIPEA (5.87 g, 45.39 mmol) in dichloromethane (20 mL) was stirred at 25° C. for 12 hours. The reaction mixture was diluted with dichloromethane and washed with H₂O. The combined organic phase was dried over Na₂SO₄, filtered and concentrated to give title compound (3.0 g crude).

2. Synthesis of compound N-(prop-2-yn-1-yl)-N-(2,2,2-trifluoroethyl)cyanamide. To a solution of N-(2,2,2-trifluoroethyl)prop-2-yn-1-amine (1 g, 7.29 mmol) in dichloromethane (8 mL) and sat. aq. NaHCO₃ (8 mL) was added BrCN (1.55 g, 14.59 mmol) in dichloromethane (8 mL) dropwise at 0° C. The reaction was stirred at 25° C. for 12 hours. The reaction was diluted with sat. aq. NaHCO₃ (10 mL) and extracted with dichloromethane (15 mL×3). The combined organic phase was dried over Na₂SO₄, filtered and concentrated. The crude product was purified by column chromatography to give title compound.

3. Synthesis of compound E192. To a solution of N-(prop-2-yn-1-yl)-N-(2,2,2-trifluoroethyl)cyanamide (61 mg, 0.38 mmol), 4-amino-7-(6-((2R,6S)-2,6-dimethylmorpholino)pyridin-3-yl)-2-(hydroxymethyl)-5,5-dimethyl-5,7-dihydro-6H-pyrrolo[2,3-d]pyrimidin-6-one (50 mg, 0.13 mmol) in 1,4-Dioxane (2 mL) was added ZnCl₂ (51 mg, 0.38 mmol) at 25° C. The reaction was stirred at 90° C. for 2 hours. The mixture was concentrated and purified by prep-HPLC to afford title compound (41 mg). ¹H NMR (400 MHz, CDCl₃) δ 8.27 (s, 1H), 7.5-7.58 (m, 1H), 6.67-6.70 (m, 1H), 5.04 (s, 2H), 4.84 (s, 2H), 4.16 (d, J=2.4 Hz, 2H), 4.10-4.11 (m, 2H), 3.97-4.04 (m, 2H), 3.68-3.76 (m, 2H), 2.54-2.60 (m, 2H), 2.28 (s, 1H), 1.52 (s, 6H), 1.28 (d, J=6.0 Hz, 6H). LCMS (ESI): m/z, 561.3 [M+H]⁺.

### Example 256. Preparation of Compound E193

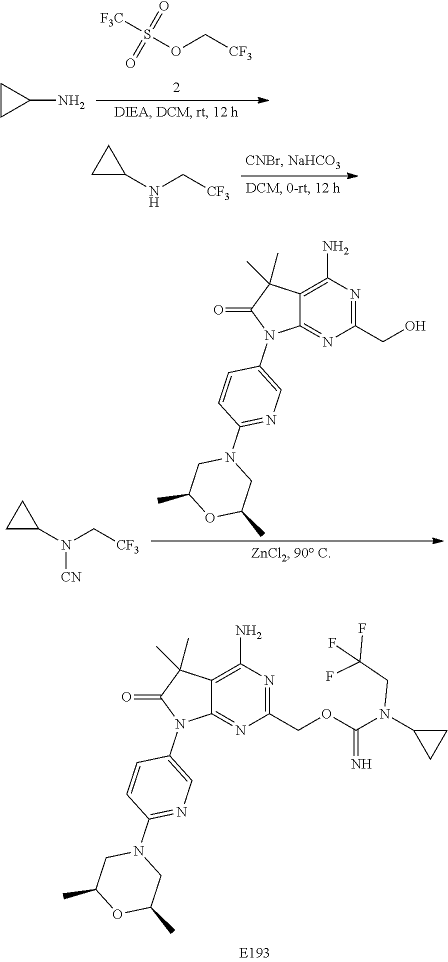

1. Synthesis of compound N-(2,2,2-trifluoroethyl)cyclopropanamine. A solution of 2,2,2-trifluoroethyl trifluoromethanesulfonate (8.13 g, 35.0 mmol), cyclopropanamine (1 g, 17.51 mmol) and DIPEA (5.66 g, 43.78 mmol) in dichloromethane (20 mL) was stirred at 25° C. for 12 hours. The reaction mixture was diluted with dichloromethane and washed with H₂O. The combined organic phase was dried over Na₂SO₄, filtered and concentrated to afford title compound (3.0 g crude).

2. Synthesis of compound N-cyclopropyl-N-(2,2,2-trifluoroethyl)cyanamide. To a solution of N-(2,2,2-trifluoroethyl)cyclopropanamine (1.0 g, 7.19 mmol) in dichloromethane (8 mL) and NaHCO₃ (8 mL) was added BrCN (1.52 g, 14.38 mmol) in dichloromethane (8 mL) dropwise. The reaction was stirred at 25° C. for 12 hours, then diluted with sat. aq. NaHCO₃ (10 mL) and extracted with dichloromethane. The combined organic phase was dried over Na₂SO₄, filtered and concentrated. The crude product was purified by column chromatography to afford title compound (500 mg). ¹H NMR (400 MHz, CDCl₃) δ 3.11-3.18 (m, 1H), 3.00-3.05 (m, 2H), 1.26 (s, 2H), 1.24 (s, 2H).

3. Synthesis of compound E193. To a solution of 4-amino-7-(6-((2R,6S)-2,6-dimethylmorpholino)pyridin-3-yl)-2-(hydroxymethyl)-5,5-dimethyl-5,7-dihydro-6H-pyrrolo[2,3-d]pyrimidin-6-one (100 mg, 0.25 mmol), N-cyclopropyl-N-(2,2,2-trifluoroethyl)cyanamide (124 mg, 0.75 mmol) in 1,4-Dioxane (2.5 mL) was added ZnCl₂ (103 mg, 0.75 mmol) at 25° C. The reaction was stirred at 90° C. for 48 hours. The mixture was concentrated and purified by prep-HPLC to afford title compound (10 mg). ¹H NMR (400 MHz, CDCl₃) δ 8.28 (d, J=2.8 Hz, 1H), 7.59 (dd, J=9.2, 2.8 Hz, 1H), 6.68 (d, J=9.2 Hz, 1H), 5.14 (s, 2H), 4.75 (s, 2H), 4.13-4.07 (m, 2H), 3.89 (q, J=10.0, 2H), 3.68-3.76 (m, 2H), 2.53-2.61 (m, 3H), 1.52 (s, 6H), 1.28 (d, J=6.4 Hz, 6H), 0.90-0.85 (m, 2H), 0.70-0.64 (m, 2H). LCMS (ESI): m/z, 563.4 [M+H]⁺.

### Example 257. Preparation of Compound E194

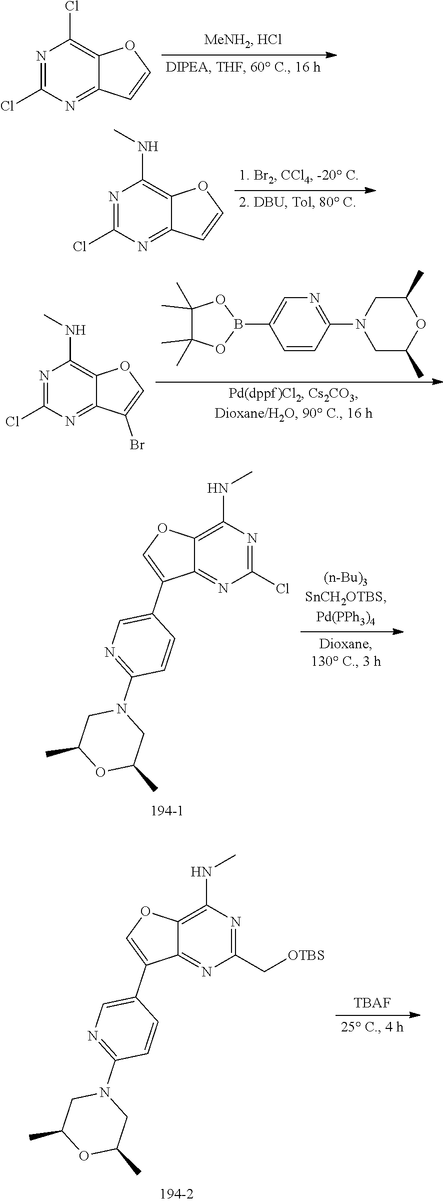

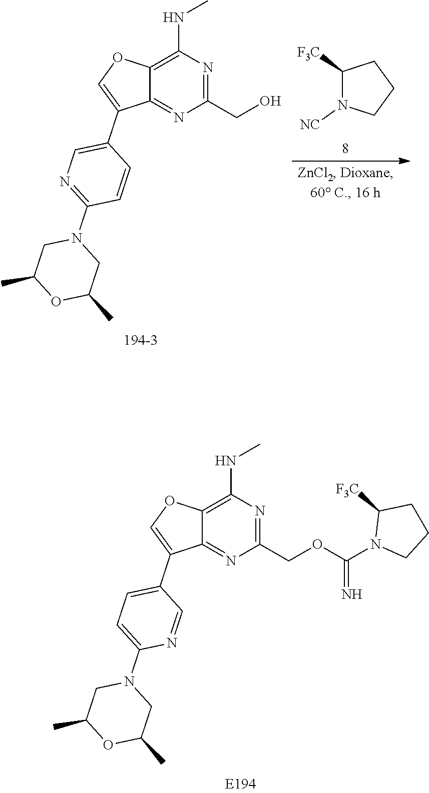 [SMILES: CNc1nc(CO)nc2c(-c3ccc(N4C[C@@H](C)O[C@@H](C)C4)nc3)coc12 ; N#CN1CCC[C@@H]1C(F)(F)F ; CNc1nc(COC(=N)N2CCC[C@@H]2C(F)(F)F)nc2c(-c3ccc(N4C[C@@H](C)O[C@@H](C)C4)nc3)coc12]

1. Synthesis of compound 2-chloro-N-methylfuro[3,2-d]pyrimidin-4-amine. To a solution of 2,4-dichlorofuro[3,2-d]pyrimidine (1.0 g, 5.29 mmol) in THF (30 mL) was added methylamine hydrochloride (893 mg, 13.2 mmol) and DIPEA (2.62 mL) at 60° C., and stirred at 60° C. for 20 hours under N₂. The reaction was diluted with water, extracted with ethyl acetate. The combined organic phases were washed with brine (15 mL), dried over anhydrous Na₂SO₄, filtered and concentrated. The residue was purified by column chromatography to afford title compound (540 mg). ¹H NMR (400 MHz, CDCl3) δ 7.72 (d, J=2.1 Hz, 1H), 6.80 (d, J=2.2 Hz, 1H), 5.28 (s, 1H), 3.24 (d, J=5.1 Hz, 3H), 1.84 (m, 4H). LCMS (ESI): m/z, 183.9, [M+H]⁺.

2. Synthesis of compound 7-bromo-2-chloro-N-methylfuro[3,2-d]pyrimidin-4-amine. To a solution of 2-chloro-N-methylfuro[3,2-d]pyrimidin-4-amine (100 mg, 0.55 mol) in CCl4 (2 mL) was added Br₂ (0.42 mL, 8.17 mmol) dropwise at −20° C., and then the reaction was stirred at 0° C. for 2 hours. The mixture was quenched with sat. Na₂SO₃ and extracted with dichloromethane, wished with brine, dried over Na₂SO₄, filtered and concentrated. Then the residue was dissolved in toluene (2 mL) and DBU (0.24 mL, 1.63 mmol) wad added. The reaction was stirred at 60° C. for 16 hours. The mixture was filtered and the filtrate was concentrated, the residue was purified by column chromatography to afford title compound (33 mg). LCMS (ESI): m/z, 261.8, 263.8 [M+H]⁺.

3. Synthesis of compound 194-1. To a mixture of 7-bromo-2-chloro-N-methylfuro[3,2-d]pyrimidin-4-amine (168 mg, 0.64 mmol) and (2S,6R)-2,6-dimethyl-4-(5-(4,4,5,5-tetramethyl-1,3,2-dioxaborolan-2-yl) pyridin-2-yl) morpholine (203.66 mg, 0.64 mmol) in 1,4-Dioxane (4 mL) and H₂O (0.5 mL) were added Pd(dppf)Cl₂ (93.28 mg, 0.13 mmol), Cs₂CO₃ (626 mg, 1.92 mmol). The mixture was degassed under vacuum and purged with N₂ several times. The reaction mixture was stirred at 90° C. for 16 hours. After cooled to room temperature, the reaction mixture was diluted with water (20 mL), and extracted with EtOAc. The organic layer was washed with brine. The solution was dried over Na₂SO₄ and filtered. The filtrate was purified by column chromatography to afford title compound (169 mg). LCMS (ESI): m/z, 374.0 [M+H]⁺.

4. Synthesis of compound 194-2. To a solution of 2-chloro-7-(6-((2R,6S)-2,6-dimethylmorpholino)pyridin-3-yl)-N-methylfuro[3,2-d]pyrimidin-4-amine (10 mg, 0.03 mmol) and Bu₃SnCH₂OTBS (12.8 mg, 0.03 mmol) in 1,4-Dioxane (2 mL) were added Pd(PPh₃)₄ (6.18 mg, 0.05 mmol) at 25° C. under N₂. The reaction was stirred at 130° C. for 3 hours in microwave. The mixture was filtered and concentrated, the residue was purified by prep-TLC to give the title compound (617 mg). LCMS (ESI): m/z, 484.3 [M+H]⁺.

5. Synthesis of compound 194-3. To a solution of 2-(((tert-butyldimethylsilyl)oxy) methyl)-7-(6-((2R,6S)-2,6-dimethylmorpholino) pyridin-3-yl)-N-methylfuro[3,2-d]pyrimidin-4-amine (122 mg, 0.25 mmol) and TBAF (2.02 mL, 1 M in THF) in THF (2 mL) under N₂. The reaction was stirred at 25° C. for 3 hours. The reaction mixture was diluted with water (10 mL), and extracted with dichloromethane. The organic layer was washed with brine, dried over Na₂SO₄_The mixture was filtered and concentrated, the residue was purified by prep-TLC to give the title compound. LCMS (ESI): m/z, 370.2 [M+H]⁺.

6. Synthesis of compound E194. To a solution of (7-(6-((2R,6S)-2,6-dimethylmorpholino) pyridin-3-yl)-4-(methylamino) furo[3,2-d]pyrimidin-2-yl) methanol (37 mg, 0.10 mmol) and (R)-2-(trifluoromethyl) pyrrolidine-1-carbonitrile (49.3 mg, 0.30 mmol) were added ZnCl₂ (54.6 mg, 0.40 mmol) in 1,4-dioxane (3 mL) at N₂. The reaction was stirred at 60° C. for 16 hours. The reaction was filtered and concentrated under reduce pressure to give a residue which was purified by prep-HPLC to give the title compound (3 mg). LCMS (ESI): m/z, 534.4 [M+H]⁺.

### Example 258. Preparation of Compound E195

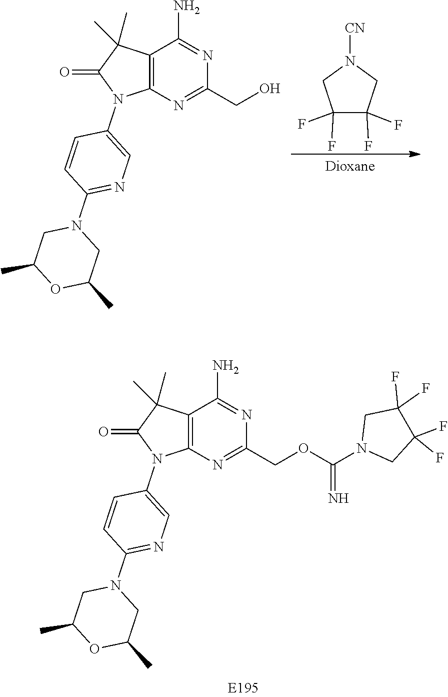 [SMILES: C[C@@H]1CN(c2ccc(N3C(=O)C(C)(C)c4c(N)nc(CO)nc43)cn2)C[C@H](C)O1 ; N#CN1CC(F)(F)C(F)(F)C1 ; C[C@@H]1CN(c2ccc(N3C(=O)C(C)(C)c4c(N)nc(COC(=N)N5CC(F)(F)C(F)(F)C5)nc43)cn2)C[C@H](C)O1]

To a solution of 3,3,4,4-tetrafluoropyrrolidine-1-carbonitrile (101 mg, 0.60 mmol) and 4-amino-7-(6-((2R,6S)-2,6-dimethylmorpholino)pyridin-3-yl)-2-(hydroxymethyl)-5,5-dimethyl-5,7-dihydro-6H-pyrrolo[2,3-d]pyrimidin-6-one (80 mg, 0.2 mmol) in 1,4-Dioxane (2 mL) was added ZnCl₂ (82 mg, 0.60 mmol) at 25° C. The reaction was stirred at 90° C. for 3 hours. The reaction mixture was concentrated and purified by prep-HPLC to afford the title compound (70 mg). ¹H NMR (400 MHz, CDCl₃) δ 8.29 (d, J=2.8 Hz, 1H), 7.57 (dd, J=9.2, 2.8 Hz, 1H), 6.68 (d, J=9.2 Hz, 1H), 5.04 (s, 2H), 4.87 (s, 2H), 4.12-4.05 (m, 2H), 3.93-3.83 (m, 4H), 3.67-3.75 (m, 2H), 2.53-2.58 (m, 2H), 1.52 (s, 6H), 1.27 (d, J=6.4 Hz, 6H). LCMS (ESI): m/z, 567.3 [M+H]⁺.

### Example 259. Preparation of Compound E196

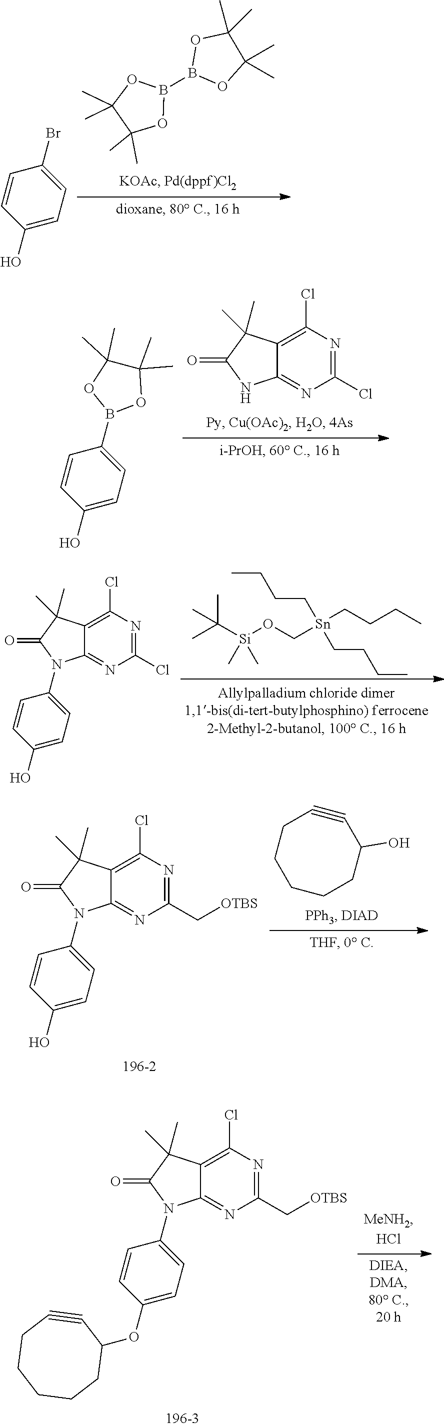

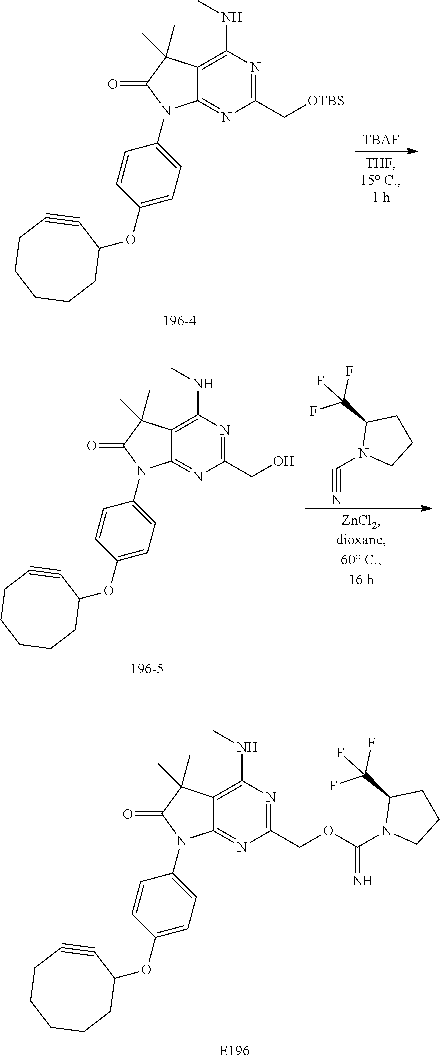

1. Synthesis of compound 4-(4,4,5,5-tetramethyl-1,3,2-dioxaborolan-2-yl)phenol. To a solution of 4-bromophenol (5 g, 28.90 mmol) in 1,4-Dioxane (100 mL) was added Pin₂B₂ (14.68 g, 57.80 mmol), KOAc (11.35 g, 115.60 mmol), Pd(dppf)Cl₂ (1.05 g, 1.45 mmol). The mixture was stirred at 80° C. for 16 hours under N2. To the mixture added ethyl acetate and H₂O, the aqueous phase was extracted with ethyl acetate. The combined organic layer were washed with brine and dried over Na₂SO₄, filtered and concentrated. The crude product was purified by column chromatography to afford the title compound (5.70 g).

2. Synthesis of compound 196-1. To a solution of 4-(4,4,5,5-tetramethyl-1,3,2-dioxaborolan-2-yl) phenol (4.27 g, 19.4 mmol) and 2,4-dichloro-5,5-dimethyl-5,7-dihydro-6H-pyrrolo[2,3-d]pyrimidin-6-one (3.0 g, 12.9 mmol) in i-PrOH (30 mL) was added DIPEA (8.55 mL, 51.7 mmol), 4 Å molecular sieve and Cu(OAc)₂·H₂O (3.36 g, 16.8 mmol). The mixture was stirred at 60° C. for 16 hours under O₂ balloon. The reaction mixture was filtered and concentrated. The crude product was purified by column chromatography to afford the title compound (710 mg).

3. Synthesis of compound 196-2. To a solution of 2,4-dichloro-7-(4-hydroxyphenyl)-5,5-dimethyl-5,7-dihydro-6H-pyrrolo[2,3-d]pyrimidin-6-one (900 mg, 2.78 mmol), allylpalladium chloride dimer (41 mg, 0.111 mmol) and 1,1′-bis(di-tert-butylphosphino) ferrocene (105 mg, 0.22 mmol) in 2-Methyl-2-butanol (15 mL) was added Bu₃SnCH₂OTBS (1.33 g, 3.05 mmol) at 25° C. The mixture was stirred at 100° C. for 16 hours under N₂. The reaction mixture was filtered and the filtrate was concentrated. The crude product was purified by column chromatography to afford the title compound (700 mg).

4. Synthesis of compound 196-3. To a solution of cyclooct-2-yn-1-ol (90 mg, 0.724 mmol), 2-(((tert-butyldimethylsilyl)oxy)methyl)-4-chloro-7-(4-hydroxyphenyl)-5,5-dimethyl-5,7-dihydro-6H-pyrrolo[2,3-d]pyrimidin-6-one (377 mg, 0.870 mmol) and PPh₃ (247 mg, 0.942 mmol) in THF (3 mL) was added DIAD (220 mg, 1.09 mmol) at 0° C. under N₂. The crude product was purified by column chromatography to afford the title compound (62 mg). ¹H NMR (400 MHz, DMSO-d6) δ 7.42-7.32 (m, 2H), 7.13-6.95 (m, 2H), 5.03 (d, J=5.6 Hz, 1H), 4.65 (s, 2H), 2.30-2.08 (m, 4H), 1.83 (dd, J=7.6, 14.0 Hz, 3H), 1.62 (ddd, J=7.6, 15.2, 23.2 Hz, 3H), 1.52 (s, 6H), 0.83 (s, 9H), 0.00 (d, J=3.2 Hz, 6H).

5. Synthesis of compound 196-4. To a solution of 2-(((tert-butyldimethylsilyl)oxy)methyl)-4-chloro-7-(4-(cyclooct-2-yn-1-yloxy)phenyl)-5,5-dimethyl-5,7-dihydro-6H-pyrrolo[2,3-d]pyrimidin-6-one (50 mg, 0.092 mmol) in DMA (4 mL) was added Methylamine hydrochloride (63 mg, 0.926 mmol), DIPEA (0.153 mL, 0.93 mmol) at 15° C. The mixture was stirred at 80° C. for 20 hours. To the mixture added ethyl acetate and H₂O, the aqueous phase was extracted with ethyl acetate. The combined organic layers were washed with brine and dried over Na₂SO₄, filtered and concentrated to give title compound without purification.

6. Synthesis of compound 196-5. To a solution of 2-(((tert-butyldimethylsilyl)oxy)methyl)-7-(4-(cyclooct-2-yn-1-yloxy)phenyl)-5,5-dimethyl-4-(methylamino)-5,7-dihydro-6H-pyrrolo[2,3-d]pyrimidin-6-one (45 mg, 0.084 mmol) in THF (4 mL) was added TBAF (1 mL, 1 M in THF). The mixture was stirred at 15° C. for 1 hour. The mixture was concentrated and the crude was purified by column chromatography to afford the title compound (20 mg).

7. Synthesis of compound E196. To a solution of (2R)-2-(trifluoromethyl)pyrrolidine-1-carbonitrile (24 mg, 0.148 mmol) and 7-(4-(cyclooct-2-yn-1-yloxy)phenyl)-2-(hydroxymethyl)-5,5-dimethyl-4-(methylamino)-5,7-dihydro-6H-pyrrolo[2,3-d]pyrimidin-6-one (20 mg, 0.049 mmol) in 1,4-Dioxane (5 mL) was added ZnCl₂ (17 mg, 0.123 mmol). The mixture was stirred at 60° C. for 16 hours. The mixture was filtered. The residue was purified by prep-HPLC to afford the title compound (8 mg). LCMS (ESI): m/z, 585.5 [M+H]⁺.

### Example 260. Preparation of Compound E197

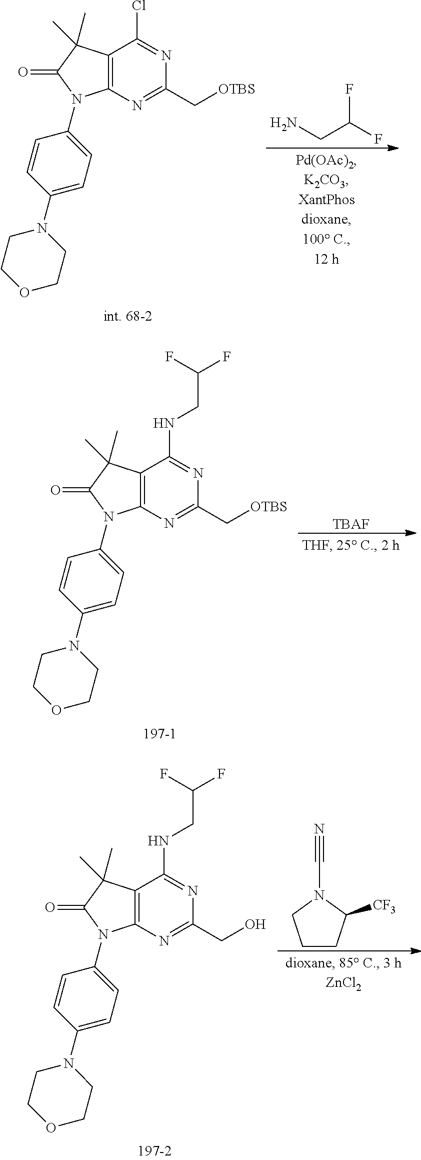

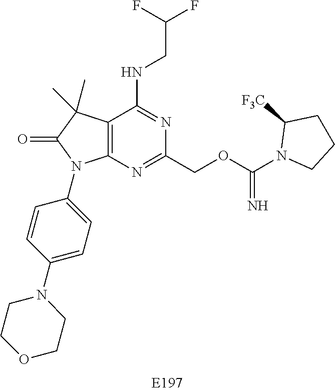 [SMILES: CC1(C)C(=O)N(c2ccc(N3CCOCC3)cc2)c2nc(COC(=N)N3CCC[C@@H]3C(F)(F)F)nc(NCC(F)F)c21]

1. Synthesis of compound 197-1. To a mixture of 2-(((tert-butyldimethylsilyl)oxy)methyl)-4-chloro-5,5-dimethyl-7-(4-morpholinophenyl)-5,7-dihydro-6H-pyrrolo[2,3-d]pyrimidin-6-one (50 mg, 0.099 mmol), XantPhos (9 mg, 0.015 mmol), K₂CO₃ (27 mg, 0.20 mmol), 2,2-difluoroethanamine (8 mg, 0.099 mmol) in 1,4-Dioxane (1 mL) was added Pd(OAc)₂ (2 mg, 0.01 mmol). The mixture was stirred at 100° C. for 12 hours. The mixture was filtered and concentrated to afford the title compound (50 mg). LCMS (ESI): m/z, 548.2 [M+H]⁺.

2. Synthesis of compound 197-2. To a solution of 2-(((tert-butyldimethylsilyl)oxy)methyl)-4-((2,2-difluoroethyl)amino)-5,5-dimethyl-7-(4-morpholinophenyl)-5,7-dihydro-6H-pyrrolo[2,3-d]pyrimidin-6-one (150 mg, 0.27 mmol) in THF (4 mL) was added TBAF (1.1 mL, 1 M in THF). The mixture was stirred at 25° C. for 2 hours. The mixture was concentrated and purified by prep-TLC to afford the title compound (55 mg). LCMS (ESI): m/z, 434.3 [M+H]⁺.

3. Synthesis of compound E197. To a mixture of (2R)-2-(trifluoromethyl)pyrrolidine-1-carbonitrile (62 mg, 0.38 mmol), 4-((2,2-difluoroethyl)amino)-2-(hydroxymethyl)-5,5-dimethyl-7-(4-morpholinophenyl)-5,7-dihydro-6H-pyrrolo[2,3-d]pyrimidin-6-one (55 mg, 0.13 mmol) in 1,4-dioxane (2 mL) was added ZnCl₂ (52 mg, 0.38 mmol). The mixture was stirred at 85° C. for 3 hours. The mixture was concentrated and purified by prep-HPLC to afford the title compound (13 mg). ¹H NMR (400 MHz, CDCl₃) δ 7.36-7.30 (m, 2H), 7.00-6.95 (m, 2H), 6.14-5.84 (m, 1H), 5.26 (d, J=14.8 Hz, 1H), 5.13 (d, J=14.8 Hz, 1H), 4.64 (d, J=6.4 Hz, 1H), 4.57-4.45 (m, 1H), 3.90-3.97 (m, 1H), 3.89-3.85 (m, 4H), 3.58-3.44 (m, 2H), 3.22-3.17 (m, 4H), 2.08-2.12 (m, 2H), 1.85-1.97 (m, 2H), 1.51 (s, 6H). LCMS (ESI): m/z, 598.4 [M+H]⁺.

### Example 261. Preparation of Compound E198

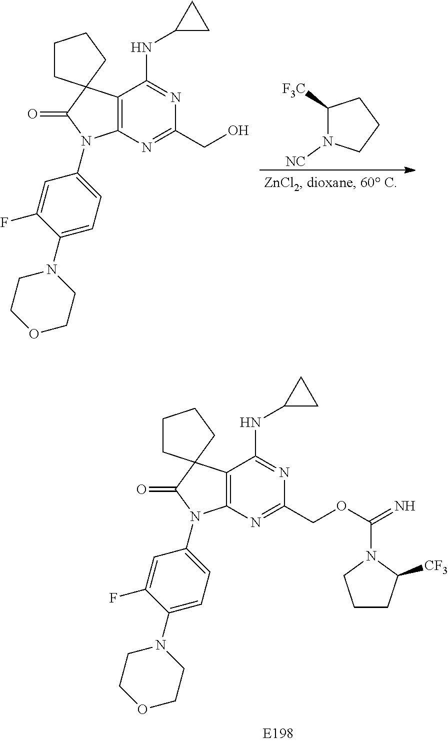 [SMILES: O=C1N(c2ccc(N3CCOCC3)c(F)c2)c2nc(CO)nc(NC3CC3)c2C12CCCC2 ; N#CN1CCC[C@@H]1C(F)(F)F ; N=C(OCc1nc(NC2CC2)c2c(n1)N(c1ccc(N3CCOCC3)c(F)c1)C(=O)C21CCCC1)N1CCC[C@@H]1C(F)(F)F]

To a solution of 4′-(cyclopropylamino)-7′-[3-fluoro-4-(morpholin-4-yl)phenyl]-2′-(hydroxymethyl)-6′,7′-dihydrospiro[cyclopentane-1,5′-pyrrolo[2,3-d]pyrimidin]-6′-one (55 mg, 0.121 mmol) in 1,4-Dioxane (1 mL) was added ZnCl₂ (41 mg, 0.303 mmol) and 2-(trifluoromethyl)pyrrolidine-1-carbonitrile (59 mg, 0.363 mmol), the mixture was stirred at 60° C. for 16 hours under N₂. The mixture was concentrated and purified by prep-HPLC to afford the title compound (35 mg). ¹H NMR (400 MHz, DMSO-dₖ) δ 9.37 (s, 1H), 7.26 (dd, J=2.2, 13.4 Hz, 1H), 7.22-7.06 (m, 2H), 6.64 (d, J=2.8 Hz, 1H), 5.38 (s, 2H), 4.82 (s, 1H), 3.76 (t, J=4.8 Hz, 4H), 3.04 (d, J=9.2 Hz, 4H), 2.78-2.76 (m, 1H), 2.17-2.15 (m, 4H), 2.05-1.92 (m, 6H), 1.82 (d, J=12.4 Hz, 2H), 1.23 (s, 2H), 0.71 (d, J=7.2 Hz, 2H), 0.59 (t, J=9.2, 13.2 Hz, 2H).

### Example 262. Preparation of Compound E199

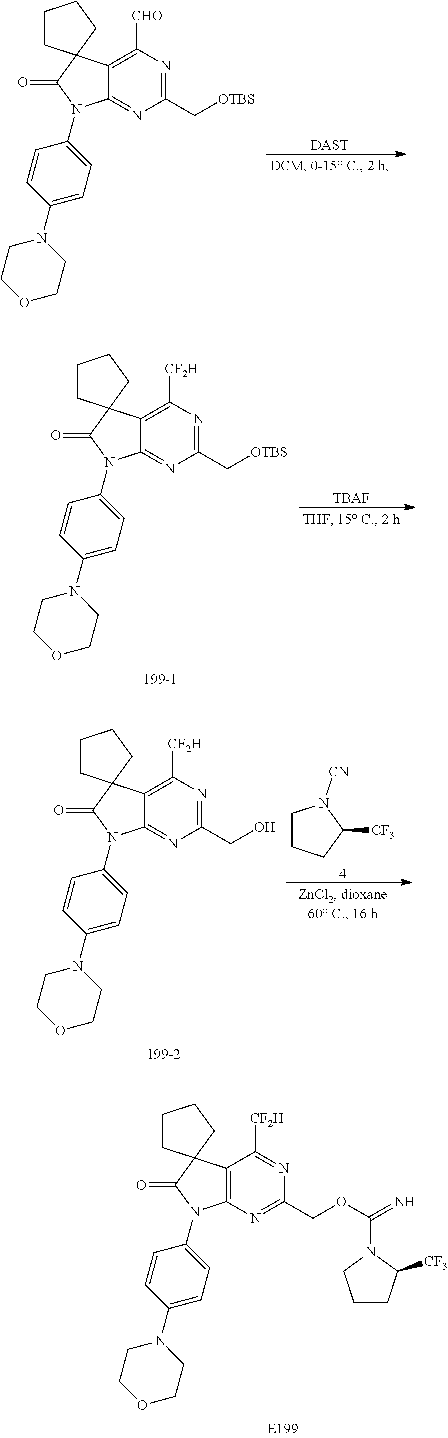

1. Synthesis of compound 199-1. To a solution of 2′-(((tert-butyldimethylsilyl)oxy)methyl)-7′-(4-morpholinophenyl)-6′-oxo-6′,7′-dihydrospiro[cyclopentane-1,5′-pyrrolo[2,3-d]pyrimidine]-4′-carbaldehyde (300 mg, 0.574 mmol) in dichloromethane (10 mL) was added DAST (120 mg, 0.746 mmol) at 0° C. The mixture was stirred at 15° C. for 2 hours. The reaction was quenched with 20 mL ice water. To the mixture was added dichloromethane and H₂O, the aqueous phase was extracted with dichloromethane. The combined organic layers were washed with brine and dried over Na₂SO₄, filtered and concentrated to afford title compound (300 mg). LCMS (ESI): m/z, 545.4 [M+H]⁺.

2. Synthesis of compound 199-2. To a solution of 2′-(((tert-butyldimethylsilyl)oxy)methyl)-4′-(difluoromethyl)-7′-(4-morpholinophenyl)spiro[cyclopentane-1,5′-pyrrolo[2,3-d]pyrimidin]-6′(7′H)-one (300 mg, 0.55 mmol) in THF (4 mL) was added TBAF (2 mL, 2 M in THF). The mixture was stirred at 15° C. for 2 hours. To the mixture added ethyl acetate and H₂O, the aqueous phase was extracted with ethyl acetate. The combined organic layers washed with brine, dried over Na₂SO₄, filtered and concentrated. The crude product was purified by column chromatography to afford the title compound (66 mg). LCMS (ESI): m/z, 431.4 [M+H]⁺.

3. Synthesis of compound E199. To a solution of 4′-(difluoromethyl)-2′-(hydroxymethyl)-7′-(4-morpholinophenyl)spiro[cyclopentane-1,5′-pyrrolo[2,3-d]pyrimidin]-6′(7′H)-one (66 mg, 0.15 mmol) and (2R)-2-(trifluoromethyl)pyrrolidine-1-carbonitrile (75 mg, 0.46 mmol) in Dioxane (3 mL) was added ZnCl₂ (52 mg, 0.38 mmol). The mixture was stirred at 60° C. for 16 hours. After filtration, the residue was purified by prep-HPLC to afford the title compound (29 mg). LCMS (ESI): m/z, 595.3 [M+H]⁺.

### Example 263. Preparation of Compound E200

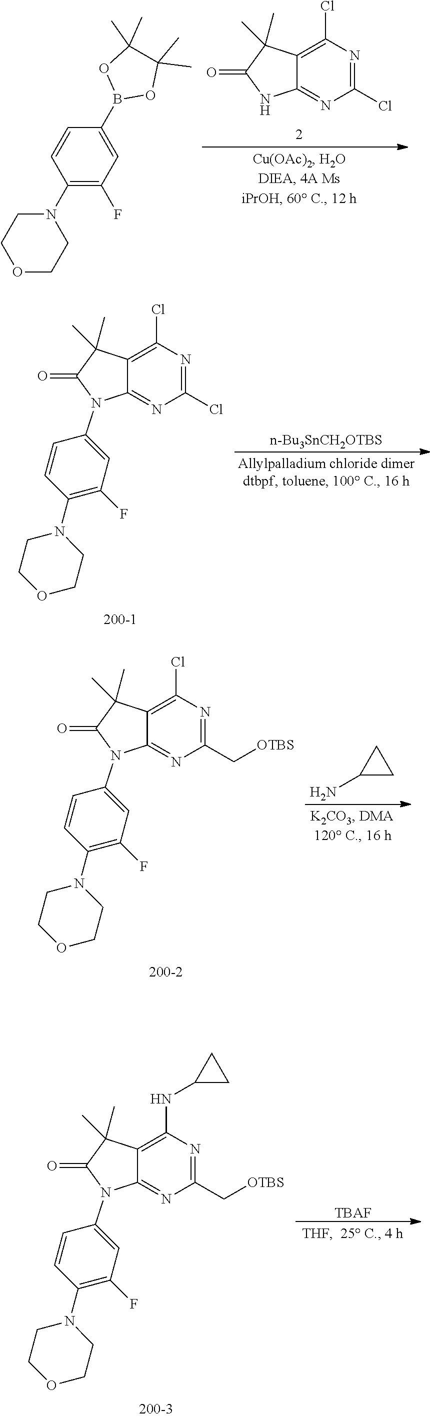

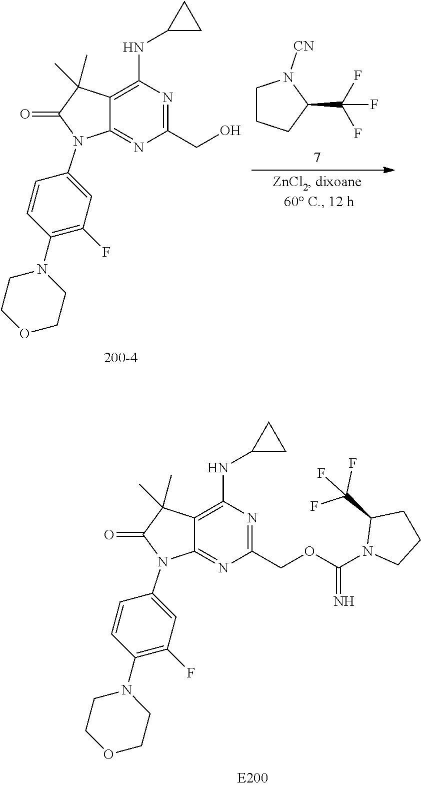

1. Synthesis of compound 200-1. To a solution of 2,4-dichloro-5,5-dimethyl-5H,6H,7H-pyrrolo[2,3-d]pyrimidin-6-one (2.0 g, 8.62 mmol) and 4-[2-fluoro-4-(4,4,5,5-tetramethyl-1,3,2-dioxaborolan-2-yl)phenyl]morpholine (5.29 g, 17.2 mmol) in i-PrOH (20 mL) was added Cu(OAc)₂·H₂O (2.06 g, 10.3 mmol), DIPEA (2.85 mL) and 4 Å Molecular Sieve (7.59 g) at 25° C. The mixture was stirred at 60° C. for 22 hours. The mixture was filtered and concentrated. The residue was purified by column chromatography to afford the title compound (1.60 g). LCMS (ESI): m/z, 411.1 [M+H]⁺.

2. Synthesis of compound 200-2. To a solution of 2,4-dichloro-7-(3-fluoro-4-morpholinophenyl)-5,5-dimethyl-5,7-dihydro-6H-pyrrolo[2,3-d]pyrimidin-6-one (1.60 g, 3.89 mmol), n-Bu₃SnCH₂OTBS (1.86 g, 4.28 mmol) in toluene (15 mL) was added allylpalladium chloride dimer (71 mg, 0.19 mmol) and 1,1′-bis(di-tert-butylphosphino) ferrocene (184 mg, 0.9 mmol) at 25° C. The reaction was stirred at 100° C. for 16 hours. The mixture was filtered and concentrated. The residue was purified by column chromatography to afford the title compound (1.5 g). LCMS (ESI): m/z, 521.1 [M+H]⁺.

3. Synthesis of compound 200-3. To a solution of 2-(((tert-butyldimethylsilyl) oxy) methyl)-4-chloro-7-(3-fluoro-4-morpholinophenyl)-5,5-dimethyl-5,7-dihydro-6H-pyrrolo[2,3-d]pyrimidin-6-one (1.40 g, 2.69 mmol) and cyclopropanamine (613.5 mg, 10.8 mmol) in DMA (15 mL) was added K₂CO₃ (1.11 g, 8.06 mmol) at 25° C. The reaction was stirred at 120° C. for 16 hours. The mixture was diluted with water, extracted with ethyl acetate, washed with brine, dried over Na₂SO₄, filtered and concentrated. The residue was purified by column chromatography to afford the title compound (1.40 g). LCMS (ESI): m/z, 542.4 [M+H]⁺.

4. Synthesis of compound 200-4. To a solution of 2-(((tert-butyldimethylsilyl) oxy) methyl)-4-(cyclopropylamino)-7-(3-fluoro-4-morpholinophenyl)-5,5-dimethyl-5,7-dihydro-6H-pyrrolo[2,3-d]pyrimidin-6-one (1.2 g) in THE (20 mL) was added TBAF (10.3 mL, 1 M in THF) at 25° C. The reaction was stirred at 25° C. for 4 hours. The reaction was concentrated directly. The residue was purified by column chromatography to afford the title compound (550 mg). LCMS (ESI): m/z, 428.2 [M+H]⁺.

5. Synthesis of compound E200. To a solution of (2R)-2-(trifluoromethyl) pyrrolidine-1-carbonitrile (115 mg, 0.70 mmol) and 4-(cyclopropylamino)-7-(3-fluoro-4-morpholinophenyl)-2-(hydroxymethyl)-5,5-dimethyl-5,7-dihydro-6H-pyrrolo[2,3-d]pyrimidin-6-one (100 mg, 0.23 mmol) in 1,4-Dioxane (4 mL) was added ZnCl₂ (127 mg, 0.94 mmol) at 60° C. for 12 hours. The mixture was filtered and concentrated. The mixture was purified by prep-HPLC to afford the title compound (55 mg). 1H NMR (400 MHz, Chloroform-d) δ 7.32-7.27 (m, 1H), 6.99 (d, J=8.8 Hz, 1H), 5.24 (d, J=14.8 Hz, 1H), 5.08 (d, J=14.8 Hz, 1H), 4.59 (d, J=2.4 Hz, 1H), 4.54-4.40 (m, 1H), 3.88 (t, J=4.4 Hz, 4H), 3.48 (dd, J=8.8, 17.2 Hz, 2H), 3.10 (dd, J=3.6, 6.4 Hz, 4H), 2.90 (m, 1H), 2.09 (d, J=8.8 Hz, 2H), 1.90 (dd, J=10.4, 18.8 Hz, 2H), 0.85 (m, 2H), 0.63-0.48 (m, 2H). LCMS (ESI): m/z, 592.4 [M+H]⁺.

### Example 264. Preparation of Compound E201

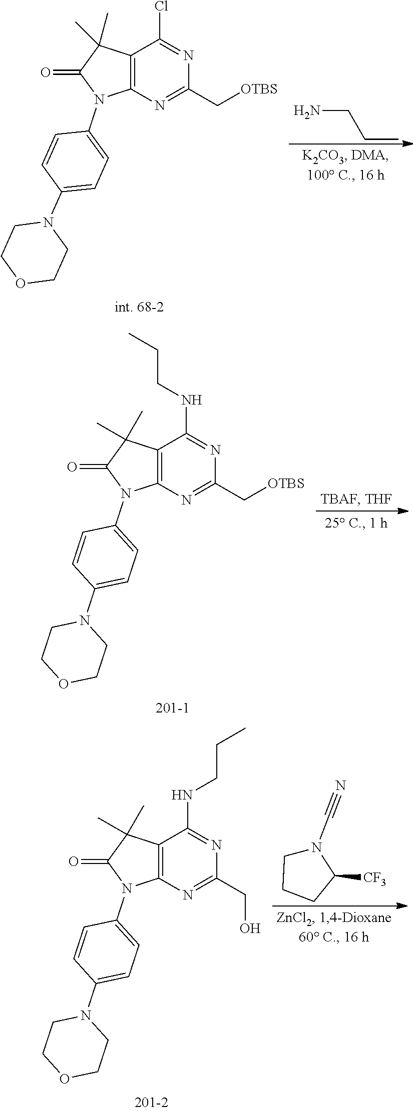

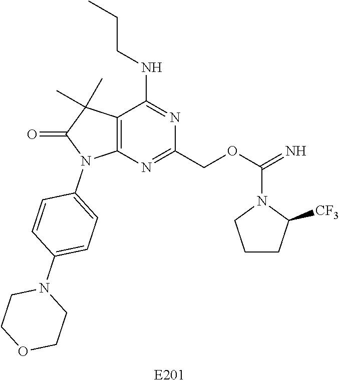 [SMILES: CCCNc1nc(COC(=N)N2CCC[C@@H]2C(F)(F)F)nc2c1C(C)(C)C(=O)N2c1ccc(N2CCOCC2)cc1]

1. Synthesis of compound 201-1. To a solution of 2-(((tert-butyldimethylsilyl) oxy) methyl)-4-chloro-5,5-dimethyl-7-(4-morpholinophenyl)-5,7-dihydro-6H-pyrrolo[2,3-d]pyrimidin-6-one (200 mg, 0.40 mmol) and 1-aminopropane (24 mg, 0.40 mmol) in DMA (5 mL) and was added K₂CO₃ (54 mg, 0.40 mmol), the mixture was heated at 100° C. for 16 hours under N₂. To the mixture added ethyl acetate (10 mL) and H₂O (20 mL), the aqueous phase was extracted with ethyl acetate. The combined organic layers were washed with brine and dried over Na₂SO₄, filtered and concentrated. The mixture was purified by column chromatography to afford the title compound (120 mg). ¹H NMR (400 MHz, CDCl₃) δ 7.39 (d, J=8.8 Hz, 2H), 6.96 (d, J=8.8 Hz, 2H), 4.64 (s, 2H), 4.33 (s, 1H), 3.87 (t, J=4.8 Hz, 4H), 3.56 (q, J=6.8 Hz, 2H), 3.18 (t, J=4.8 Hz, 4H), 1.69-1.61 (m, 2H), 1.49 (s, 6H), 1.00 (s, 3H), 0.95 (d, J=26.0 Hz, 9H), 0.08 (s, 6H).

2. Synthesis of compound 201-2. To a solution of 2-(((tert-butyldimethylsilyl) oxy) methyl)-5,5-dimethyl-7-(4-morpholinophenyl)-4-(propylamino)-5,7-dihydro-6H-pyrrolo[2,3-d]pyrimidin-6-one (120 mg, 0.23 mmol) in THF (10 mL) was added TBAF (1.0 mL, 1 M in THF), the mixture was stirred at 25° C. for 1 hour. To the mixture added ethyl acetate and H₂O, the aqueous phase was extracted with ethyl acetate. The combined organic layers were washed with brine and dried over Na₂SO₄, filtered and concentrated. The residue was purified by column chromatography to afford the title compound (12 mg). LCMS (ESI): m/z, 412.3 [M+H]⁺.

3. Synthesis of compound E201. To a solution of 2-(hydroxymethyl)-5,5-dimethyl-7-(4-morpholinophenyl)-4-(propylamino)-5,7-dihydro-6H-pyrrolo[2,3-d]pyrimidin-6-one (12 mg, 0.03 mmol) and 2-(trifluoromethyl)pyrrolidine-1-carbonitrile (7.2 mg, 0.044 mmol) was added ZnCl₂ (9 mg, 0.07 mmol), in 1,4-dioxane (2 mL) at N₂. The reaction was stirred at 60° C. for 16 hours. The mixture was filtered and concentrated. The residue was purified by prep-HPLC to afford the title compound (7 mg). LCMS (ESI): m/z, 576.4 [M+H]⁺.

### Example 265. Preparation of Compound E202

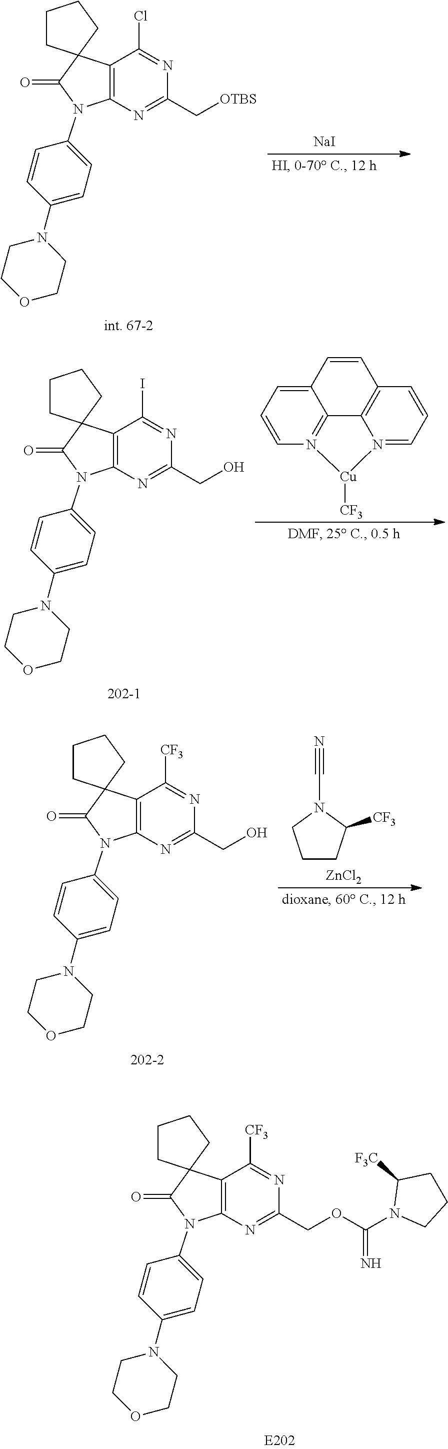

1. Synthesis of compound 202-1. A solution of 2′-(((tert-butyldimethylsilyl)oxy)methyl)-4′-chloro-7′-(4-morpholinophenyl)spiro[cyclopentane-1,5′-pyrrolo[2,3-d]pyrimidin]-6′(7′H)-one (400 mg, 0.76 mmol) and NaI (2.27 g, 15.12 mmol) in HI (10 mL) was stirred at 70° C. for 12 h. The reaction mixture was concentrated and basified by saturated NaHCO₃ to pH 7. The aqueous phase was extracted with ethyl acetate. The combined organic phase was washed with brine, dried over anhydrous Na₂SO₄, filtered and concentrated. The residue was purified by column chromatography to afford the title compound (250 mg). LCMS (ESI): m/z, 507.1 [M+H]⁺.

2. Synthesis of compound 202-2. To a solution of 2′-(hydroxymethyl)-4′-iodo-7′-(4-morpholinophenyl)spiro[cyclopentane-1,5′-pyrrolo[2,3-d]pyrimidin]-6′(7′H)-one (250 mg, 0.49 mmol) in DMF (4 mL) was added Trifluoromethyl(1,10-phenanthroline)copper (309 mg, 0.99 mmol). The solution was stirred at 25° C. for 0.5 hour. The solution was diluted with ethyl acetate (50 mL) and washed with brine. The organic phase was dried over anhydrous Na₂SO₄, filtered and concentrated. The residue was purified by column chromatography to afford the title compound (100 mg). LCMS (ESI): m/z, 449.1 [M+H]⁺.

3. Synthesis of compound E202. To a mixture of 2′-(hydroxymethyl)-7′-[4-(morpholin-4-yl)phenyl]-4′-(trifluoromethyl)-6′,7′-dihydrospiro[cyclopentane-1,5′-pyrrolo[2,3-d]pyrimidin]-6′-one (85 mg, 0.19 mmol), (2R)-2-(trifluoromethyl)pyrrolidine-1-carbonitrile (93 mg, 0.57 mmol) in THF (2 mL) was added ZnCl₂ (78 mg, 0.57 mmol). The mixture was stirred at 60° C. for 12 hours. The mixture was concentrated and purified by prep-HPLC to afford the title compound (40 mg). 1H NMR (400 MHz, DMSO) δ 7.30 (d, J=8.8 Hz, 2H), 7.05 (d, J=9.2 Hz, 2H), 5.88 (s, 1H), 5.15-5.27 (m, 2H), 4.58-4.47 (m, 1H), 3.76 (t, J=4.8 Hz, 4H), 3.23-3.31 (m, 2H), 3.18 (t, J=4.8 Hz, 4H), 2.27-2.18 (m, 2H), 2.11-1.97 (m, 4H), 1.95-1.77 (m, 6H). LCMS (ESI): m/z, 613.3 [M+H]⁺.

### Example 266. Preparation of Compound E203

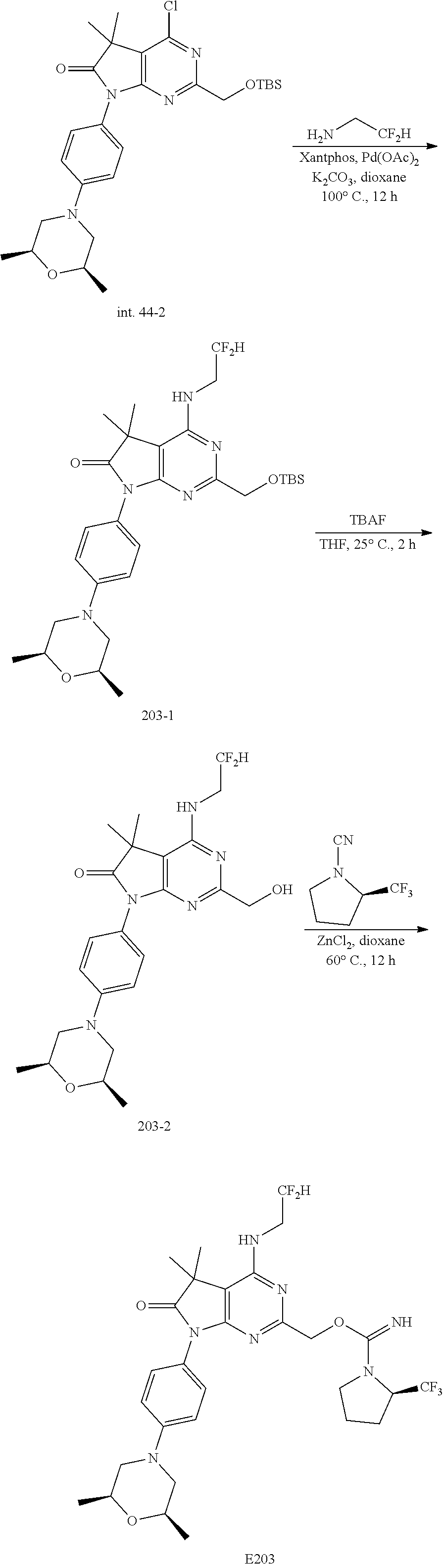

1. Synthesis of compound 203-1. A mixture of 2-(((tert-butyldimethylsilyl)oxy)methyl)-4-chloro-7-(4-((2R,6S)-2,6-dimethylmorpholino)phenyl)-5,5-dimethyl-5,7-dihydro-6H-pyrrolo[2,3-d]pyrimidin-6-one (800 mg, 1.51 mmol), 2,2-difluoroethanamine (610 mg, 7.53 mmol), XantPhos (87 mg, 0.150 mmol), Pd(OAc)₂ (17 mg, 0.075 mmol), K₂CO₃ (416 mg, 3.01 mmol) in 1,4-Dioxane (15 mL) was stirred at 100° C. for 12 hours under N₂ atmosphere. The mixture was filtered and concentrated to afford crude title compound (900 mg). LCMS (ESI): m/z, 576.4 [M+H]⁺.

2. Synthesis of compound 203-2. To a solution of 2-(((tert-butyldimethylsilyl)oxy)methyl)-4-((2,2-difluoroethyl)amino)-7-(4-((2R,6S)-2,6-dimethylmorpholino)phenyl)-5,5-dimethyl-5,7-dihydro-6H-pyrrolo[2,3-d]pyrimidin-6-one (900 mg) in THF (15 mL) was added TBAF (2.81 mL, 1 M in THF). The mixture was stirred at 25° C. for 12 hours. The mixture was concentrated. The residue was purified by column chromatography to afford the title compound (400 mg). LCMS (ESI): m/z, 446.2 [M+H]⁺.

3. Synthesis of compound E203. To a solution of 4-((2,2-difluoroethyl)amino)-7-(4-((2R,6S)-2,6-dimethylmorpholino)phenyl)-2-(hydroxymethyl)-5,5-dimethyl-5,7-dihydro-6H-pyrrolo[2,3-d]pyrimidin-6-one (400 mg, 0.866 mmol) in 1,4-Dioxane (10 mL) was added ZnCl₂ (236 mg, 1.73 mmol). The mixture was stirred at 60° C. for 12 hours under N₂ atmosphere. The mixture was filtered and concentrated. The residue was purified by prep-HPLC to afford the title compound (100 mg) as white solid.

¹H NMR (400 MHz, CDCl₃) δ 7.33 (d, J=9.2 Hz, 2H), 6.94 (d, J=9.2 Hz, 2H), 6.12-5.83 (m, 1H), 5.21-5.05 (m, 2H), 4.59 (t, J=6.4 Hz, 1H), 4.46-4.42 (m, 1H), 3.97-3.76 (m, 4H), 3.50-3.38 (m, 4H), 2.47-2.42 (m, 2H), 2.11-2.05 (m, 2H), 1.97-1.85 (m, 2H), 1.50 (s, 6H), 1.27 (d, J=6.4 Hz, 6H). LCMS (ESI): m/z, 626.4 [M+H]⁺.

### Example 267. Preparation of Compound E204

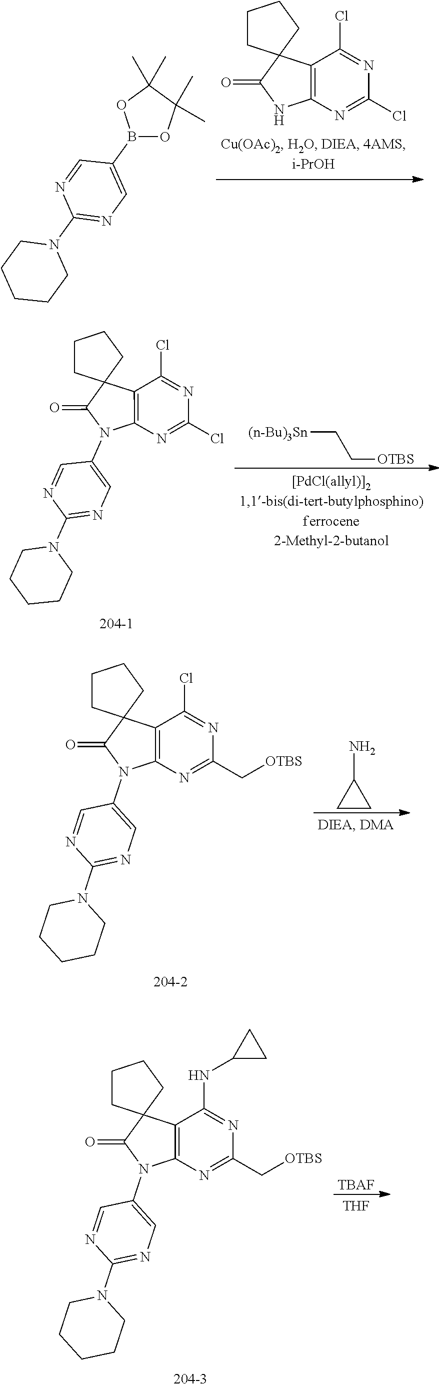

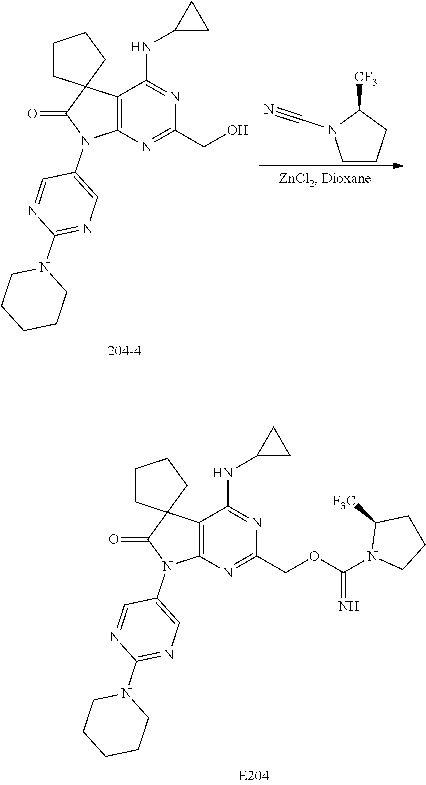

1. Synthesis of compound 204-1. To a solution of 2′,4′-dichlorospiro[cyclopentane-1,5′-pyrrolo[2,3-d]pyrimidin]-6′(7′H)-one (3 g, 11.62 mmol) in i-PrOH (30 mL) was added 2-(piperidin-1-yl)-5-(4,4,5,5-tetramethyl-1,3,2-dioxaborolan-2-yl)pyrimidine (3.36 g, 11.62 mmol), Cu(OAc)₂ (3.17 g, 17.44 mmol), DIPEA (4.51 g, 5.76 mL, 34.87 mmol) and 4 Å Molecular Sieve (5.12 g) at 25° C. The reaction was stirred at 60° C. for 16 hours under O₂. The mixture was filtered and concentrated. The residue was purified by column chromatography to afford the title compound (4.30 g). LCMS (ESI): m/z, 419.2 [M+H]⁺.

2. Synthesis of compound 204-2. To a solution of 2′,4′-dichloro-7′-(2-(piperidin-1-yl)pyrimidin-5-yl)spiro[cyclopentane-1,5′-pyrrolo[2,3-d]pyrimidin]-6′(7′H)-one (3.78 g, 9.01 mmol) in 2-Methyl-2-butanol (15 mL) was added tert-butyldimethyl((tributylstannyl)methoxy)silane (4.32 g, 9.92 mmol), 1,1′-bis(di-tert-butylphosphino)ferrocene (342 mg, 0.72 mmol) and [PdCl(allyl)]2 (132 mg, 0.36 mmol) at 25° C. The reaction was stirred at 110° C. for 16 hours. The mixture was filtered and concentrated. The residue was purified by column chromatography to afford the title compound (3.20 g). LCMS (ESI): m/z, 529.3 [M+H].

3. Synthesis of compound 204-3. To a solution of cyclopropylamine (1.62 g, 1.96 mL, 28.35 mmol) and 2′-(((tert-butyldimethylsilyl)oxy)methyl)-4′-chloro-7′-(2-(piperidin-1-yl)pyrimidin-5-yl)spiro[cyclopentane-1,5′-pyrrolo[2,3-d]pyrimidin]-6′(7′H)-one (1.50 g, 2.83 mmol) in DMA (30 mL) was added DIPEA (2.34 mL, 14.17 mmol) under N₂. The mixture was stirred at 110° C. for 16 hours. To the mixture added ethyl acetate and H₂O, the aqueous phase was extracted with ethyl acetate. The combined organic layers were washed with brine and dried over Na₂SO₄, filtered and concentrated. The crude product was used in next step without further purification.

4. Synthesis of compound 204-4. To a solution of 2′-(((tert-butyldimethylsilyl) oxy) methyl)-4′-(cyclopropylamino)-7′-(2-(piperidin-1-yl) pyrimidin-5-yl) spiro[cyclopentane-1,5′-pyrrolo[2,3-d]pyrimidin]-6′(7′H)-one (1.50 g, 2.73 mmol) in THF (15 mL) was added TBAF (4 mL, 1 M in THF). The mixture was stirred at 20° C. for 2 hours. The mixture was filtered and concentrated. The residue was purified by column chromatography to afford the title compound (450 mg). LCMS (ESI): m/z, 436.5 [M+H]⁺.

5. Synthesis of compound E204. To a solution of (R)-2-(trifluoromethyl)pyrrolidine-1-carbonitrile (396 mg, 2.41 mmol) and 4′-(cyclopropylamino)-2′-(hydroxymethyl)-7′-(2-(piperidin-1-yl)pyrimidin-5-yl)spiro[cyclopentane-1,5′-pyrrolo[2,3-d]pyrimidin]-6′(7′H)-one (350 mg, 0.80 mmol) in 1,4-dioxane (5 mL) was added ZnCl₂ (328 mg, 2.41 mmol) and stirred at 60° C. for 16 hours under N₂ atmosphere. The mixture was filtered and concentrated. The mixture was purified by prep-HPLC to afford the title compound (20 mg). ¹H NMR (400 MHz, CDCl3) δ 8.39 (s, 2H), 5.25 (d, J=15.2 Hz, 1H), 5.08 (d, J=14.8 Hz, 1H), 4.49 (d, J=7.6 Hz, 2H), 3.99-3.65 (m, 4H), 3.49 (d, J=9.6 Hz, 2H), 2.91 (d, J=8.0 Hz, 1H), 2.21-1.58 (m, 19H), 0.92-0.79 (m, 2H), 0.54 (s, 2H). LCMS (ESI): m/z, 600.4 [M+H]⁺.

### Example 268. Preparation of Compound E205

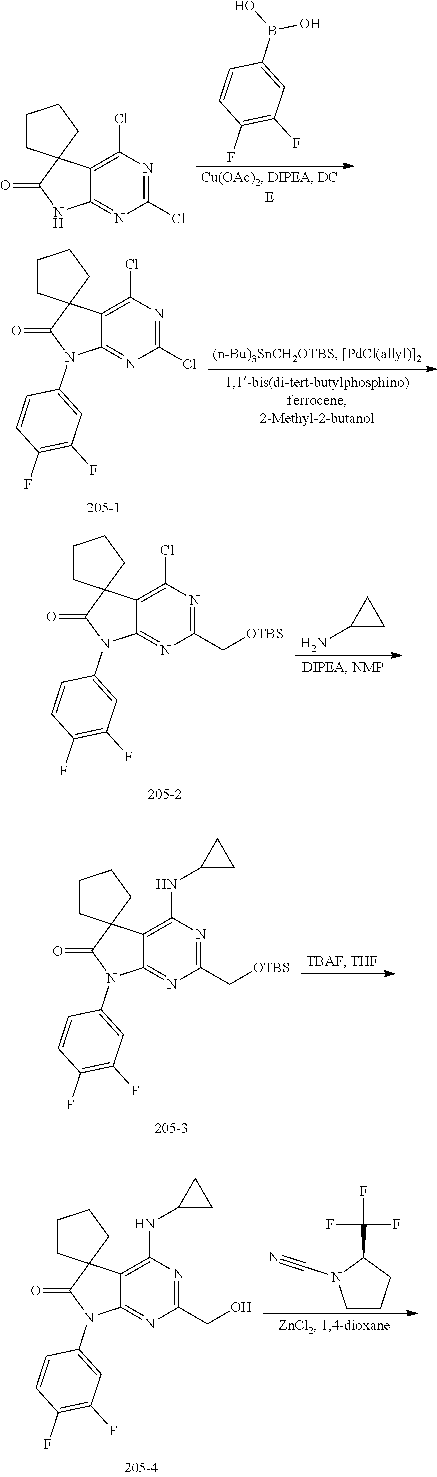

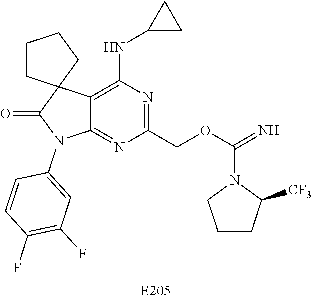 [SMILES: N=C(OCc1nc(NC2CC2)c2c(n1)N(c1ccc(F)c(F)c1)C(=O)C21CCCC1)N1CCC[C@@H]1C(F)(F)F]

1. Synthesis of compound 205-1. To a solution of 2′,4′-dichlorospiro[cyclopentane-1,5′-pyrrolo[2,3-d]pyrimidin]-6′(7′H)-one (100 mg, 0.39 mmol) in dichloroethane (2 mL) was added (3,4-difluorophenyl)boronic acid (61 mg, 0.39 mmol), Cu(OAc)₂ (84 mg, 0.47 mmol) and DIPEA (0.19 mL) at 25° C. The reaction was stirred at 60° C. for 16 hours under O₂. The reaction mixture was filtered and concentrated. The residue was purified by prep-TLC to afford the title compound (110 mg). ¹H NMR (400 MHz, CDCl₃) (δ 7.40-7.35 (m, 1H), 7.33-7.26 (m, 2H), 2.38-2.24 (m, 2H), 2.23-2.04 (m, 6H). LCMS (ESI): m/z, 370.0 [M+H]⁺.

2. Synthesis of compound 205-2. To a solution of 2′,4′-dichloro-7′-(3,4-difluorophenyl)spiro[cyclopentane-1,5′-pyrrolo[2,3-d]pyrimidin]-6′(7′H)-one (3.26 g, 7.05 mmol) in 2-Methyl-2-butanol (30 mL) was added (tert-butyldimethylsilyloxymethyl)tri-n-butyltin (3.99 g, 9.16 mmol), [PdCl(allyl)]₂ (258 mg, 0.71 mmol) and 1,1′-bis(di-tert-butylphosphino)ferrocene (668 mg, 1.41 mmol) at 25° C. The reaction was stirred at 100° C. for 20 hours under N₂. The mixture was concentrated and purified by column chromatography to afford the title compound (2.50 g). LCMS (ESI): m/z, 480.1 [M+H]⁺.

3. Synthesis of compound 205-3. To a solution of 2′-(((tert-butyldimethylsilyl)oxy)methyl)-4′-chloro-7′-(3,4-difluorophenyl)spiro[cyclopentane-1,5′-pyrrolo[2,3-d]pyrimidin]-6′(7′H)-one (2.30 g, 4.79 mmol) in NMP (10 mL) was added DIPEA (2.38 mL) and cyclopropylamine (2.74 g, 47.91 mmol) at 25° C. The reaction was stirred at 110° C. for 16 hours. After cooled to 25° C., the reaction mixture was diluted with water (50 mL) and extracted with ethyl acetate. The organic layer was washed with brine, dried over Na₂SO₄. The filtrate was concentrated to afford the title compound (2.30 g). LCMS (ESI): m/z, 501.3 [M+H]⁺.

4. Synthesis of compound 205-4. To a solution of 2′-(((tert-butyldimethylsilyl)oxy)methyl)-4′-(cyclopropylamino)-7′-(3,4-difluorophenyl)spiro[cyclopentane-1,5′-pyrrolo[2,3-d]pyrimidin]-6′(7′H)-one (2.30 g, 4.59 mmol) in THE (8 mL) was added TBAF (18.4 mL, 1 M in THF) at 25° C. The reaction was stirred at 25° C. for 1 hour. The mixture was quenched with sat. NH₄Cl solution, diluted with water (40 ml), and extracted with ethyl acetate. The organic layer was washed with brine, dried over Na₂SO₄. The filtrate was concentrated, the residue was purified by column chromatography to afford the title compound (810 mg). LCMS (ESI): m/z, 387.2 [M+H]⁺.

5. Synthesis of compound E205. To a solution of 4′-(cyclopropylamino)-7′-(3,4-difluorophenyl)-2′-(hydroxymethyl) spiro[cyclopentane-1,5′-pyrrolo [2,3-d]pyrimidin]-6′(7′H)-one (330 mg, 0.85 mmol) in 1,4-dioxane (5 mL) was added (2R)-2-(trifluoromethyl)pyrrolidine-1-carbonitrile (420 mg, 2.56 mmol) and ZnCl₂ (466 mg, 3.42 mmol) at 25° C. The reaction was stirred at 60° C. for 16 hours. The reaction was filtered and concentrated. The residue was purified by prep-HPLC to afford the title compound (68.7 mg). 1H NMR (400 MHz, Chloroform-d) δ 7.47 (m, 1H), 7.37-7.31 (m, 1H), 7.25-7.18 (m, 1H), 5.24 (d, J=14.8 Hz, 1H), 5.09 (d, J=14.8 Hz, 1H), 4.55-4.40 (m, 2H), 3.53-3.42 (m, 2H), 2.92-2.91 (m, 1H), 2.14 (dd, J=8.4, 12.0 Hz, 6H), 2.03-1.92 (m, 4H), 1.90-1.84 (m, 2H), 0.90-0.83 (m, 2H), 0.58-0.51 (m, 2H). LCMS (ESI): m/z, 551.3 [M+H]⁺.

### Example 269. Preparation of Compound E206

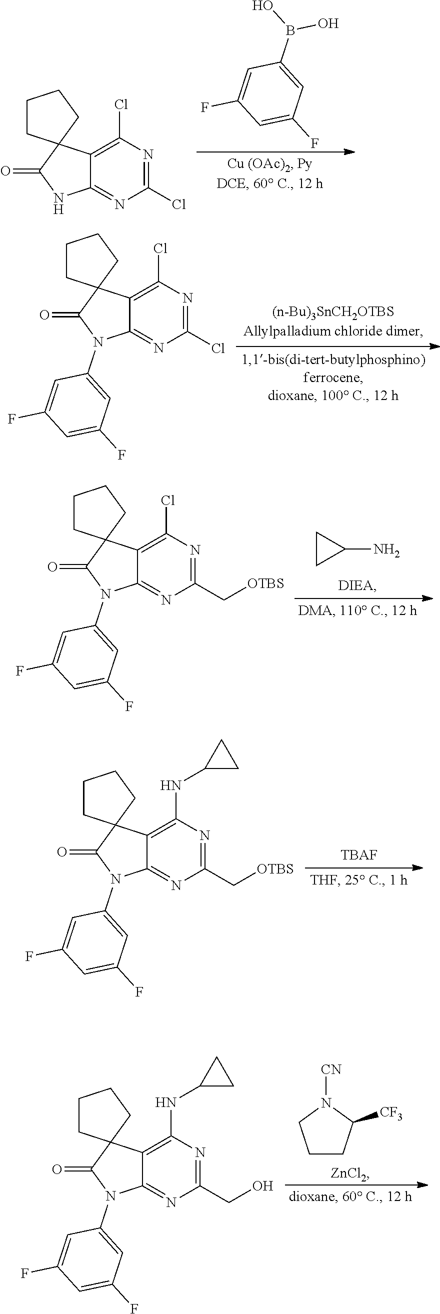

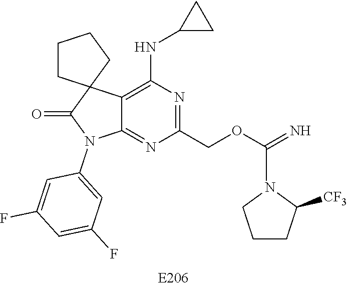

Compound E206 was synthesized as compound E53. ¹H NMR (400 MHz, DMSO) δ 7.36-7.27 (m, 3H), 6.45 (s, 1H), 5.78 (s, 1H), 5.08-4.92 (m, 2H), 4.57 (t, J=8.0 Hz, 1H), 2.85 (dq, J=7.0, 3.4 Hz, 1H), 2.24-1.74 (m, 14H), 0.72-0.70 (m, 2H), 0.61-0.54 (m, 2H). LCMS (ESI): m/z, 551.3 [M+H]⁺.

### Example 270. Preparation of Compound E207

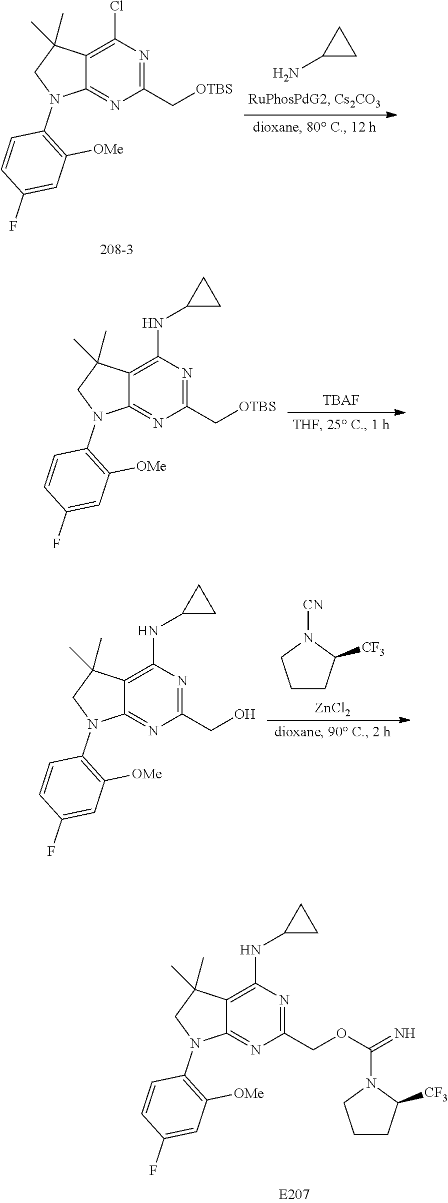

Compound E207 was synthesized as compound E16. ¹H NMR (400 MHz, DMSO) δ 7.30-7.34 (m, 1H), 6.60-6.67 (m, 2H), 5.08 (d, J=14.0 Hz, 1H), 4.91 (d, J=14.0 Hz, 1H), 4.52-4.56 (m, 1H), 4.42-4.34 (m, 1H), 3.78 (s, 3H), 3.61-3.40 (m, 4H), 2.85-2.89 (m, 1H), 2.04-2.06 (m, 2H), 1.92-1.84 (m, 2H), 1.35 (s, 6H), 0.77-0.82 (m, 2H), 0.56-0.46 (m, 2H). LCMS (ESI): m/z, 523.3 [M+H]⁺.

### Example 271. Preparation of Compound E208

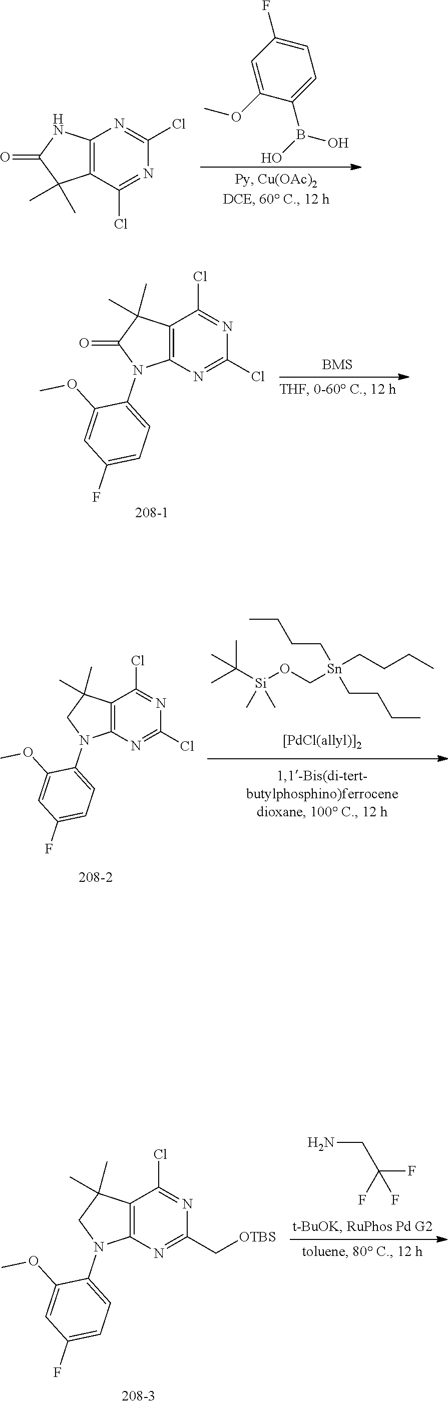

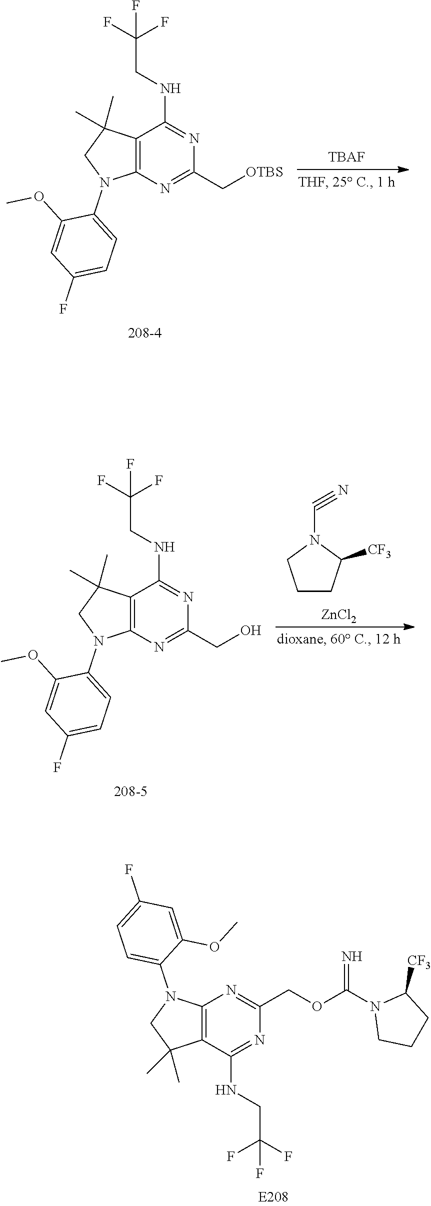

1. Synthesis of compound 208-1. To a mixture of 2,4-dichloro-5,5-dimethyl-5,7-dihydro-6H-pyrrolo[2,3-d]pyrimidin-6-one (3.0 g, 12.9 mmol), (4-fluoro-2-methoxyphenyl)boronic acid (4.39 g, 25.9 mmol), 4As (5.69 g), DIPEA (5.01 g, 38.8 mmol) in DCE (60 mL) was added Cu(OAc)₂ (2.58 g, 14.2 mmol). The mixture was stirred at 60° C. for 12 hours under O₂. The mixture was filtered and concentrated. The crude product was purified by column chromatography to give title compound (1.02 g). LCMS (ESI): m/z, 356.0 [M+H]⁺.

2. Synthesis of compound 208-2. To a solution of 2,4-dichloro-7-(4-fluoro-2-methoxyphenyl)-5,5-dimethyl-5,7-dihydro-6H-pyrrolo[2,3-d]pyrimidin-6-one (1.95 g, 5.47 mmol) in THF (35 mL) was dropwise added BMS (2.08 g, 27.37 mmol, 2 M in THF) at 0° C., the mixture was stirred at 60° C. for 12 hours. The mixture was quenched with MeOH (30 mL) at 0° C. and stirred at 25° C. for 0.5 hour, then concentrated. The crude product was purified by column chromatography to give title compound (920 mg). LCMS (ESI): m/z, 342.1 [M+H]⁺.

3. Synthesis of compound 208-3. A mixture of 2,4-dichloro-7-(4-fluoro-2-methoxyphenyl)-5,5-dimethyl-6,7-dihydro-5H-pyrrolo[2,3-d]pyrimidine (920 mg, 2.69 mmol), tert-butyldimethyl((tributylstannyl)methoxy)silanen (1.29 g, 2.96 mmol), [PdCl(allyl)]₂ (49 mg, 0.13 mmol) and 1,1′-bis(di-tert-butylphosphino)ferrocene (128 mg, 0.27 mmol) in 2-Methyl-2-butanol (25 mL) was stirred at 100° C. for 12 hours under N₂ atmosphere. The mixture was filtered and concentrated. The crude product was purified by column chromatography to give title compound (386 mg). LCMS (ESI): m/z, 452.3 [M+H]⁺.

4. Synthesis of compound 208-4. A mixture of 2-(((tert-butyldimethylsilyl)oxy)methyl)-4-chloro-7-(4-fluoro-2-methoxyphenyl)-5,5-dimethyl-6,7-dihydro-5H-pyrrolo[2,3-d]pyrimidine (320 mg, 0.71 mmol), 2,2,2-trifluoroethan-1-amine (701 mg, 7.08 mmol), t-BuOK (159 mg, 1.42 mmol) and RuPhos Pd G2 (110 mg, 0.14 mmol) in toluene (5 mL) was stirred at 80° C. for 12 hours under N₂ atmosphere. The mixture was filtered and concentrated, then diluted with EA and washed with water and brine, the organic layer was dried over Na₂SO₄, filtered and concentrated to give title compound (440 mg crude).

5. Synthesis of compound 208-4. To a solution of 2-(((tert-butyldimethylsilyl)oxy)methyl)-7-(4-fluoro-2-methoxyphenyl)-5,5-dimethyl-N-(2,2,2-trifluoroethyl)-6,7-dihydro-5H-pyrrolo[2,3-d]pyrimidin-4-amine (440 mg, 0.85 mmol) in THF (8 mL) was added TBAF (523 mg, 2 mmol), the mixture was stirred at 25° C. for 1.5 hours. The mixture was diluted with EA and washed with water and brine, the organic layer was dried over Na₂SO₄, filtered and concentrated. The crude product was purified by column chromatography to give title compound (113 mg). LCMS (ESI): m/z, 401.1 [M+H]⁺.

6. Synthesis of compound E208. A mixture of (7-(4-fluoro-2-methoxyphenyl)-5,5-dimethyl-4-((2,2,2-trifluoroethyl)amino)-6,7-dihydro-5H-pyrrolo[2,3-d]pyrimidin-2-yl)methanol (87 mg, 0.22 mmol), (R)-2-(trifluoromethyl)pyrrolidine-1-carbonitrile (107 mg, 0.65 mmol) and ZnCl₂ (89 mg, 0.65 mmol) in 1,4-dioxane (2.5 mL) was stirred at 90° C. for 3 hours under N₂ atmosphere. The mixture was filtered and concentrated. The residue was purified by preparative HPLC to give title compound (88 mg). ¹H NMR (400 MHz, CDCl3) δ 7.31 (dd, J=8.4, 6.2 Hz, 1H), 6.70-6.60 (m, 2H), 5.06 (d, J=14.6 Hz, 1H), 4.91 (d, J=14.4 Hz, 1H), 4.53-4.45 (m, 1H), 4.36-4.22 (m, 3H), 3.79 (s, 3H), 3.60 (s, 2H), 3.51-3.40 (m, 2H), 2.11-2.04 (m, 2H), 1.96-1.86 (m, 2H), 1.41 (s, 6H). LCMS (ESI): m/z, 565.2 [M+H]⁺.

### Example 272. Preparation of Compound E209

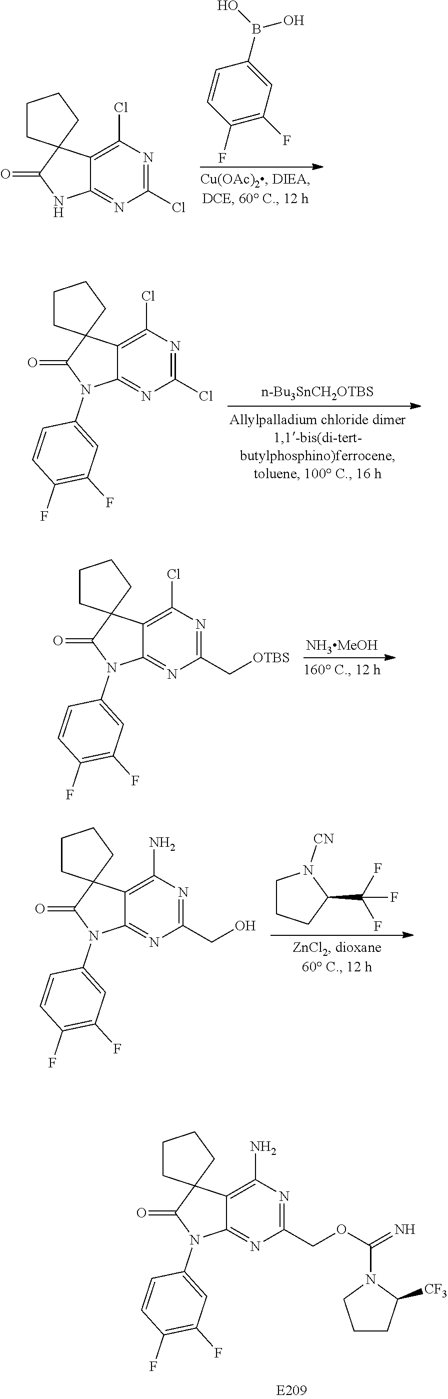

Compound E209 was synthesized as compound E34. ¹H NMR (400 MHz, CDCl₃) δ 7.45-7.50 (in, InH), 7.37-7.31 (in, InH), 7.25-7.17 (in, I H), 5.21-5.02 (m, 2H), 4.79 (s, 2H), 4.40-4.48 (in, I H), 3.52-3.38 (m, 2H), 2.20-2.04 (m, 8H), 2.00-1.89 (in, 4H). LCMS (ESI): m/z, 511.2 [M+H]⁺.

### Example 273. Preparation of Compound E210

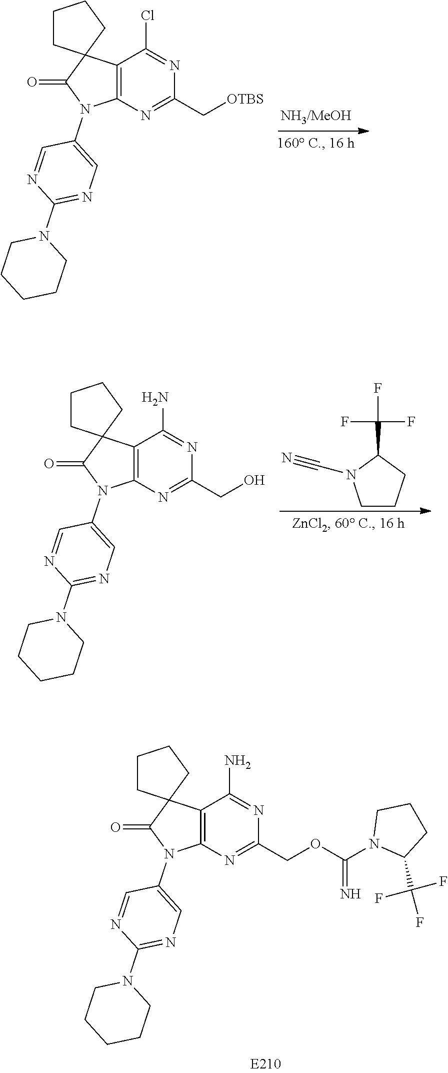

Compound E210 was synthesized as compound E33. ¹H NMR (400 MHz, CDCl₃) δ 8.39 (s, 2H), 5.19 (d, J=14.8 Hz, 1H), 5.06-4.99 (m, 1H), 4.78 (s, 2H), 4.45 (t, J=7.6 Hz, 1H), 3.84-3.79 (m, 4H), 3.49-3.43 (m, 2H), 2.18-2.05 (m, 8H), 1.91 (s, 4H), 1.71-1.66 (m, 2H), 1.64-1.58 (m, 4H). LCMS (ESI): m/z, 536.2 [M+H]⁺.

### Example 274. Preparation of Compound E211

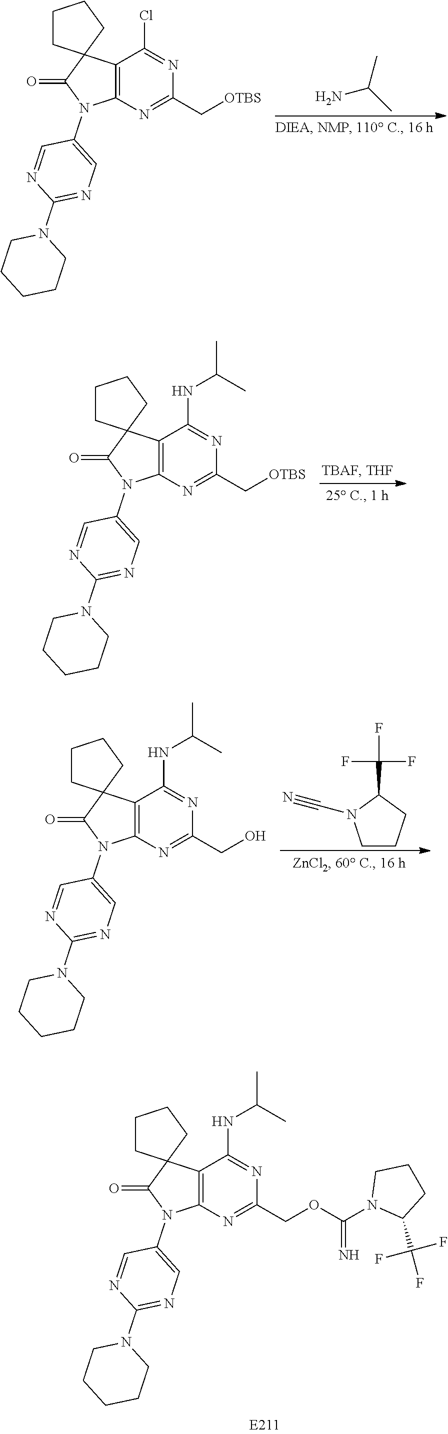

Compound E211 was synthesized as compound E204. ¹H NMR (400 MHz, CDCl₃) δ 8.40 (s, 2H), 5.19 (d, J=15.2 Hz, 1H), 5.02 (d, J=15.2 Hz, 1H), 4.50-4.34 (m, 2H), 4.08 (s, 1H), 3.84-3.77 (m, 5H), 3.47 (t, J 9.2 Hz, 2H), 2.16-1.94 (m, 10H), 1.90-1.83 (m, 2H), 1.68 (d, J -5.6 Hz, 2H), 1.62-1.59 (m, 4H), 1.24 (d, J=6.4 Hz, 6H). LCMS (ESI): m/z, 602.6 [M+H]⁺.

### Example 275. Preparation of Compound E212

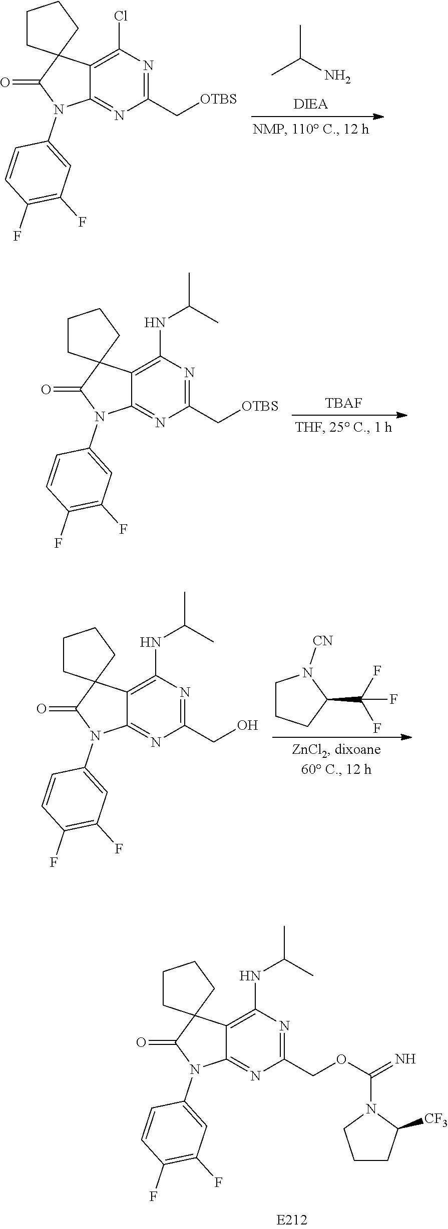

Compound E212 was synthesized as compound E40. ¹H NMR (400 MHz, CDCl₃) δ 7.44-7.50 (m, 1H), 7.31-7.36 (m, 1H), 7.25-7.18 (m, 1H), 5.21-5.02 (m, 2H), 4.35-4.51 (m, 2H), 4.09 (d, J=7.6 Hz, 1H), 3.54-3.41 (m, 2H), 2.09-2.20 (m, 6H), 2.03-1.86 (m, 6H), 1.25 (d, J=6.4 Hz, 6H). LCMS (ESI): m/z, 553.3 [M+H]⁺.

### Example 276. Preparation of Compound E213

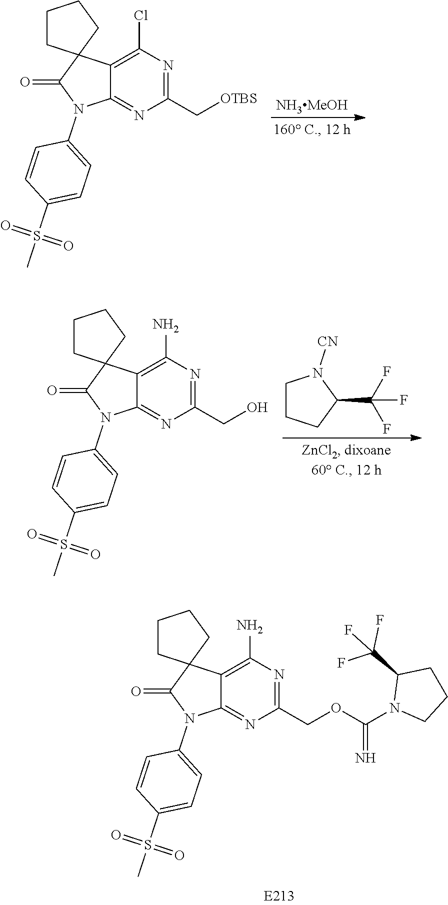

Compound E213 was synthesized as compound E33. ¹H NMR (400 MHz, CDCl₃) δ 8.05-7.98 (m, 2H), 7.89-7.83 (m, 2H), 5.18 (d, J=14.4 Hz, 1H), 5.07 (d, J=14.4 Hz, 1H), 4.91 (s, 2H), 4.48-4.36 (m, 1H), 3.55-3.37 (m, 2H), 3.07 (s, 3H), 2.21-2.08 (m, 8H), 1.94 (s, 4H). LCMS (ESI): m/z, 553.2 [M+H]⁺.

### Example 277. Preparation of Compound E214

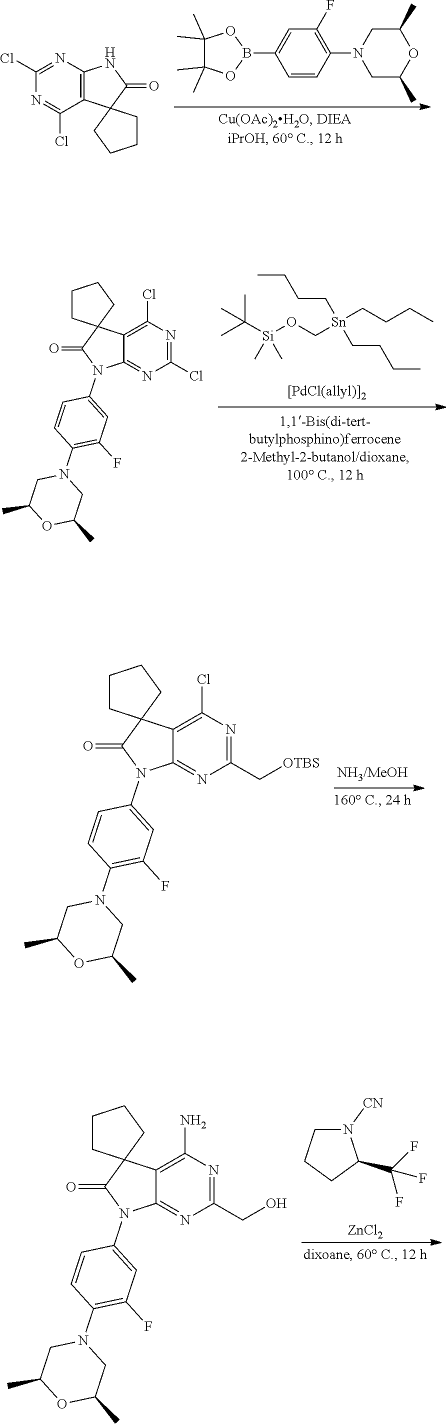

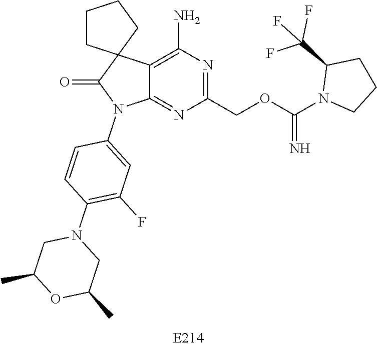 [SMILES: C[C@@H]1CN(c2ccc(N3C(=O)C4(CCCC4)c4c(N)nc(COC(=N)N5CCC[C@@H]5C(F)(F)F)nc43)cc2F)C[C@H](C)O1]

Compound E214 was synthesized as compound E33. ¹H NMR (400 MHz, CDCl₃) δ 7.27 (d, J=2.4 Hz, 1H), 7.24 (s, 1H), 6.96 (t, J=8.8 Hz, 1H), 5.18 (d, J=14.2 Hz, 1H), 5.04 (d, J=14.2 Hz, 1H), 4.84 (s, 2H), 4.47 (q, J=8.0 Hz, 1H), 3.87 (dt, J=3.6, 9.2 Hz, 2H), 3.53-3.44 (m, 2H), 3.33-3.25 (m, 2H), 2.51-2.43 (m, 2H), 2.19-2.05 (m, 8H), 1.93 (d, J=14.0 Hz, 4H), 1.24 (dd, J=6.4, 0.8 Hz, 6H). LCMS (ESI): m/z, 606.3 [M+H]⁺.

### Example 278. Preparation of Compound E215

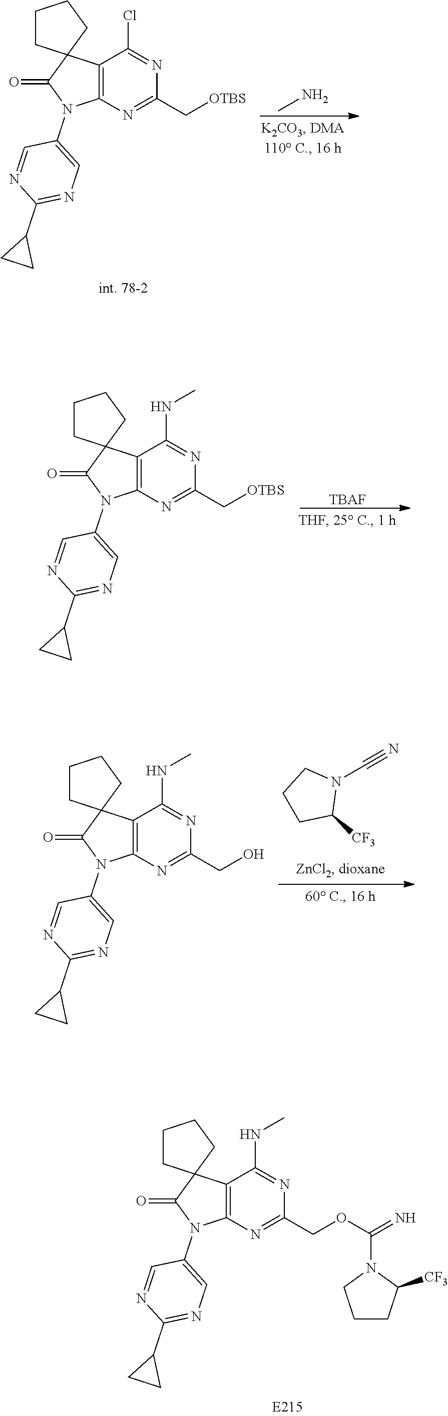

Compound E215 was synthesized as compound E22. ¹H NMR (400 MHz, Chloroform-d) b 8.82 (s, 2H), 5.22 (d, J=15.2 Hz, 1H), 5.07 (d, J=15.2 Hz, 1H), 4.45 (t, J=7.6 Hz, 1H), 4.31 (d, J=5.2 Hz, 1H), 3.49 (d, J=8.4 Hz, 2H), 3.09 (d, J=4.4 Hz, 3H), 2.33-2.25 (m, 1H), 2.19-1.84 (m, 13H), 1.17-1.06 (m, 4H). LCMS (ESI): m/z, 531.6 [M+H]⁺.

### Example 279. Preparation of Compound E216

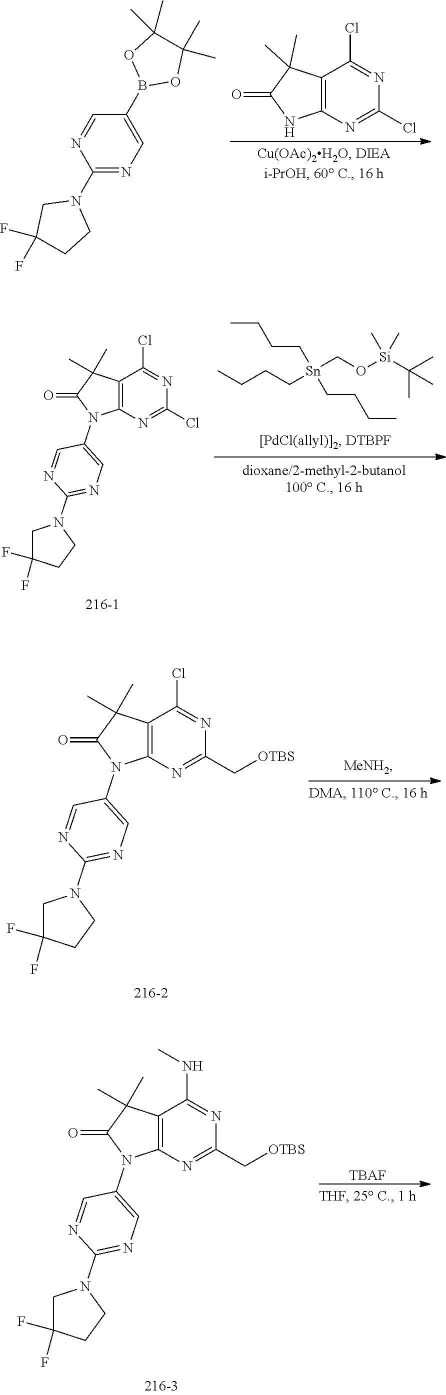

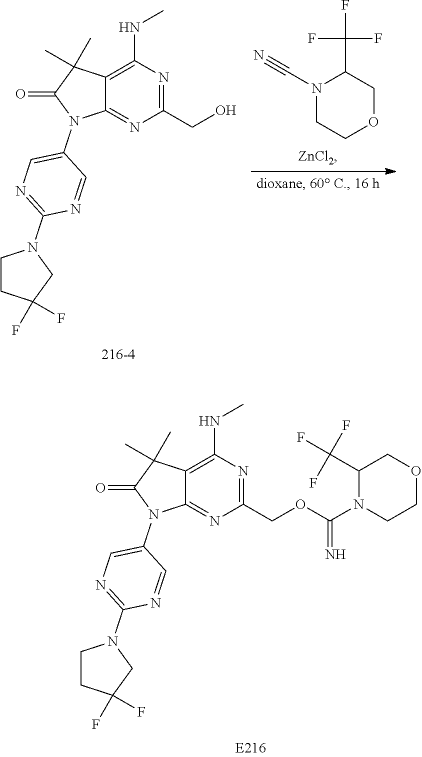 [SMILES: CNc1nc(CO)nc2c1C(C)(C)C(=O)N2c1cnc(N2CCC(F)(F)C2)nc1 ; N#CN1CCOCC1C(F)(F)F ; CNc1nc(COC(=N)N2CCOCC2C(F)(F)F)nc2c1C(C)(C)C(=O)N2c1cnc(N2CCC(F)(F)C2)nc1]

1. Synthesis of compound 216-1. A mixture of 2-(3,3-difluoropyrrolidin-1-yl)-5-(4,4,5,5-tetramethyl-1,3,2-dioxaborolan-2-yl)pyrimidine (3.33 g, 12.9 mmol), 2-(3,3-difluoropyrrolidin-1-yl)-5-(4,4,5,5-tetramethyl-1,3,2-dioxaborolan-2-yl)pyrimidine (6.03 g, 19.4 mmol), DIPEA (5.01 g, 38.8 mmol), Cu(OAc)₂·H₂O (2.84 g, 14.2 mmol) and 4 Å Molecular Sieve (5.69 g) in i-PrOH (60 mL) was stirred at 60° C. for 16 h under 02. The reaction mixture was filtered and the filtrate dried in vacuum to give the residue. The residue was purified by column chromatography to afford title compound (4.2 g). LCMS (ESI): mi/z, 415.0 [M+H].

2. Synthesis of compound 216-2. A mixture of 2,4-dichloro-7-(2-(3,3-difluoropyrrolidin-1-yl)pyrimidin-5-yl)-5,5-dimethyl-5,7-dihydro-6H-pyrrolo[2,3-d]pyrimidin-6-one (2.30 g, 5.54 mmol), (tert-butyldimethylsilyloxymethyl)tri-n-butyltin (2.65 g, 6.09 mmol), [PdCl(allyl)]2 (101 mg, 0.28 mmol) and 1,1′-bis(di-tert-butylphosphino)ferrocene (263 mg, 0.55 mmol) in 1,4-Dioxane (10 mL) and 2-Methyl-2-butanol (30 mL) was stirred at 100° C. for 16 hours under N₂. The reaction mixture was dried in vacuum to give the residue. The residue was purified by column chromatography to afford title compound (1.1 g). LCMS (ESI): 525.2 [M+H]-.

3. Synthesis of compound 216-3. A mixture of 2-(((tert-butyldimethylsilyl)oxy)methyl)-4-chloro-7-(2-(3,3-difluoropyrrolidin-1-yl)pyrimidin-5-yl)-5,5-dimethyl-5,7-dihydro-6H-pyrrolo[2,3-d]pyrimidin-6-one (1.10 g, 2.09 mmol), K₂CO₃ (1.45 g, 10.5 mmol) and methanamine hydrochloride (707 mg, 10.5 mmol) in DMA (10 mL) was stirred at 110° C. for 16 hours. To the mixture added EtOAc and H₂O, the aqueous phase was extracted with EtOAc. The combined organic layers were washed with brine and dried over Na₂S₄, filtered and concentrated. The reaction mixture was dried in vacuum to afford title compound (1.0 g crude).

4. Synthesis of compound 216-4. To a mixture of 2-(((tert-butyldimethylsilyl)oxy)methyl)-7-(2-(3,3-difluoropyrrolidin-1-yl)pyrimidin-5-yl)-5,5-dimethyl-4-(methylamino)-5,7-dihydro-6H-pyrrolo[2,3-d]pyrimidin-6-one (1.0 g, 1.92 mmol) in THF (20 mL) was added TBAF (3.85 mL, 1 M in THF). The reaction mixture was stirred at 25° C. for 1 hour. To the mixture was added EA and H₂O, the aqueous phase was extracted with EA. The combined organic layers were washed with brine and dried over Na₂SO₄, filtered and concentrated. The residue was purified by column chromatography to afford title compound (400 mg). LCMS (ESI): m/z, 406.1 [M+H]⁺.

5. Synthesis of compound E216. To a solution of 3-(trifluoromethyl)morpholine-4-carbonitrile (355.45 mg, 1.97 mmol, 2 eq), 7-(2-(3,3-difluoropyrrolidin-1-yl)pyrimidin-5-yl)-2-(hydroxymethyl)-5,5-dimethyl-4-(methylamino)-5,7-dihydro-6H-pyrrolo[2,3-d]pyrimidin-6-one (400 mg, 0.99 mmol) and ZnCl₂ (269 mg, 1.97 mmol) in 1,4-Dioxane (7 mL) was stirred at 60° C. for 16 hours under N₂. The reaction mixture was filtered and the filtrate was concentrated. The crude product was purified by prep-HPLC to afford title compound (180 mg). ¹H NMR (400 MHz, DMSO-d₆) δ 9.57 (s, 2H), 8.47 (s, 2H), 7.01-7.00 (m, 1H), 5.40 (s, 2H), 4.86-4.60 (m, 1H), 4.27-3.74 (m, 9H), 2.91 (d, J=4.0 Hz, 3H), 2.61-2.54 (m, 2H), 1.45 (s, 6H). LCMS (ESI): m/z, 586.5 [M+H]⁺.

### Example 280. Preparation of Compound E217

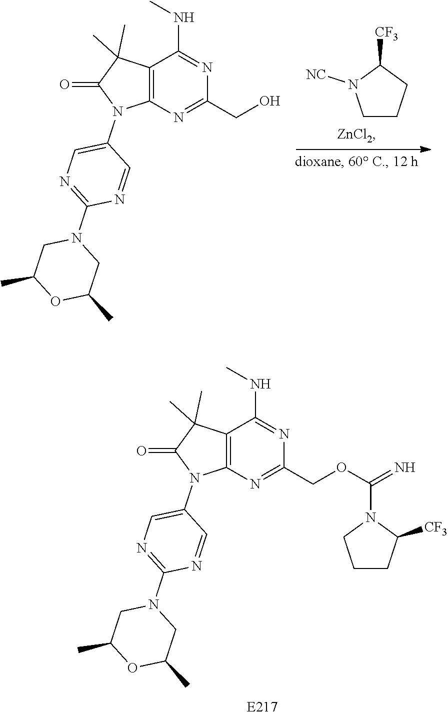 [SMILES: CNc1nc(CO)nc2c1C(C)(C)C(=O)N2c1cnc(N2C[C@@H](C)O[C@@H](C)C2)nc1 ; N#CN1CCC[C@@H]1C(F)(F)F ; CNc1nc(COC(=N)N2CCC[C@@H]2C(F)(F)F)nc2c1C(C)(C)C(=O)N2c1cnc(N2C[C@@H](C)O[C@@H](C)C2)nc1]

Compound E217 was synthesized as compound E16. ¹H NMR (400 MHz, CDCl₃) δ: 8.46 (s, 2H), 5.24-5.06 (m, 2H), 4.61-4.58 (m, 2H), 4.52-4.43 (m, 2H), 3.68-3.59 (m, 2H), 3.53-3.48 (m, 2H), 3.08 (d, J=9.2 Hz, 1H), 2.66-2.60 (m, 2H), 2.13-1.97 (m, 4H), 1.49 (s, 6H), 1.27 (d, J=6.4 Hz, 1H). LCMS (ESI): m/z, 578.5 [M+H]⁺.

### Example 281. Preparation of Compound E218

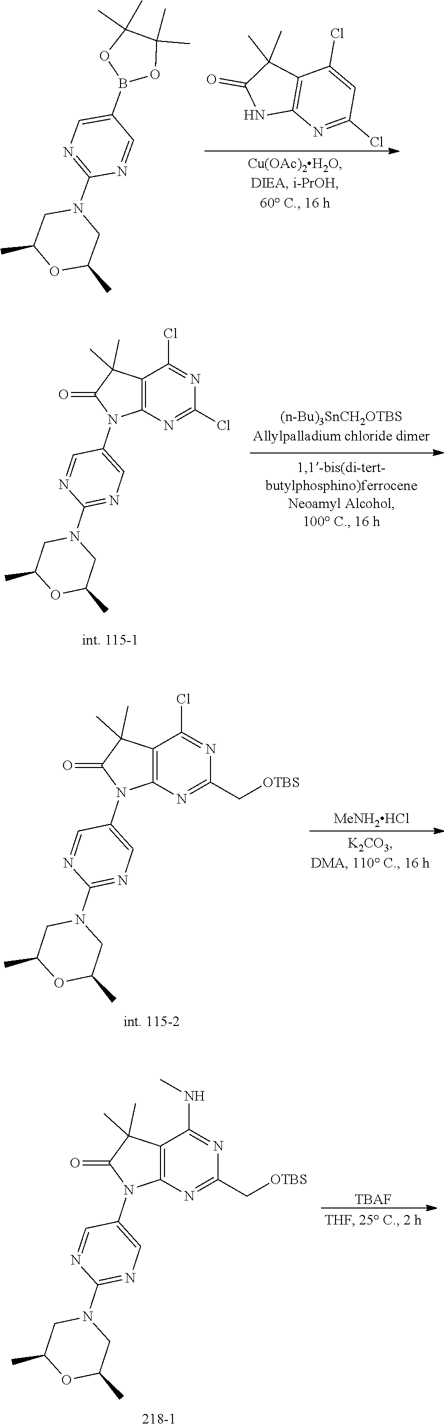

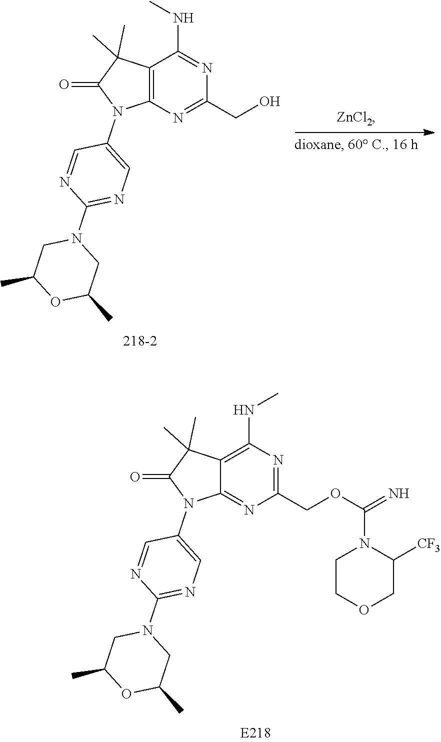 [SMILES: CNc1nc(CO)nc2c1C(C)(C)C(=O)N2c1cnc(N2C[C@@H](C)O[C@@H](C)C2)nc1 ; CNc1nc(COC(=N)N2CCOCC2C(F)(F)F)nc2c1C(C)(C)C(=O)N2c1cnc(N2C[C@@H](C)O[C@@H](C)C2)nc1]

1. Synthesis of compound 115-1. To a solution of 2,4-dichloro-5,5-dimethyl-5H,6H,7H-pyrrolo[2,3-d]pyrimidin-6-one (7.0 g, 27.1 mmol, 90%) and 2,6-dimethyl-4-(5-(4,4,5,5-tetramethyl-[1,3,2]dioxaborolan-2-yl)pyrimidin-2-yl)morpholine (13.0 g, 40.7 mmol) in i-PrOH (150 mL) were added Cu(OAc)₂·H₂O (5.96 g, 29.9 mmol), DIPEA (13.5 mL) and 4A Molecular Sieve (12.0 g). The mixture was stirred at 60° C. for 16 hours under O₂. The mixture was filtered and the filtrate was concentrated. The residue was purified by column chromatography to give title compound (5.70 g). LCMS (ESI): m/z, 423.1 [M+H]⁺.

2. Synthesis of compound 115-2. To a solution of 2,4-dichloro-7-{2-[(2R,6S)-2,6-dimethylmorpholin-4-yl]pyrimidin-5-yl}-5,5-dimethyl-5H,6H,7H-pyrrolo[2,3-d]pyrimidin-6-one (5.70 g, 13.47 mmol) and (tert-butyldimethylsilyloxymethyl)tri-n-butyltin (6.45 g, 14.81 mmol) in 2-Methyl-2-butanol (100 mL) were added [PdCl(allyl)]₂ (246 mg, 0.67 mmol) and 1,1′-bis(di-tert-butylphosphino)ferrocene (639 mg, 1.35 mmol). The mixture was stirred at 100° C. for 16 hours under N₂. The solvent was removed to yield a residue which was purified by column chromatography to give title compound (1.80 g). LCMS (ESI): m/z, 533.4 [M+H]⁺.

3. Synthesis of compound 218-1. To a solution of 2-{[(tert-butyldimethylsilyl)oxy]methyl}-4-chloro-7-{2-[(2R,6S)-2,6-dimethylmorpholin-4-yl]pyrimidin-5-yl}-5,5-dimethyl-5H,6H,7H-pyrrolo[2,3-d]pyrimidin-6-one (750 mg, 1.41 mmol, 1 eq) in DMA (10 mL) were added K₂CO₃ (972 mg, 7.03 mmol, 5 eq) and MeNH₂ HCl salt (475 mg, 7.03 mmol, 5 eq). The mixture was stirred at 110° C. for 16 hours. The reaction was sat up two batches. Water was added and extracted with EtOAc. The combined organic phases were washed with brine, dried over anhydrous Na₂SO₄, filtered and concentrated to give title compound (1.48 g crude).

4. Synthesis of compound 218-2. To a solution of 2-{[(tert-butyldimethylsilyl)oxy]methyl}-7-{2-[(2R,6S)-2,6-dimethylmorpholin-4-yl]pyrimidin-5-yl}-5,5-dimethyl-4-(methylamino)-5H,6H,7H-pyrrolo[2,3-d]pyrimidin-6-one (1.48 g, 2.80 mmol) in THF (10 mL) was added TBAF (5.61 mL, 1 M in THF). The mixture was stirred at 25° C. for 2 hours. Water was added and extracted with EtOAc. The combined organic phases were washed with brine, dried over anhydrous Na₂SO₄, filtered and concentrated. The residue was purified by column chromatography to give title compound (470 mg). LCMS (ESI): m/z, 414.4 [M+H]⁺.

5. Synthesis of compound E218. To a solution of 7-{2-[(2R,6S)-2,6-dimethylmorpholin-4-yl]pyrimidin-5-yl}-2-(hydroxymethyl)-5,5-dimethyl-4-(methylamino)-5H,6H,7H-pyrrolo[2,3-d]pyrimidin-6-one (270 mg, 0.65 mmol) and 3-(trifluoromethyl)morpholine-4-carbonitrile (117 mg, 0.65 mmol) in 1,4-dioxane (10 mL) and THF (10 mL) was added ZnCl₂ (1.96 mL, 1 M in THF). The mixture was stirred at 60° C. for 16 hours under N₂. The mixture was filtered and the filtrate was concentrated. The residue was purified by prep-HPLC to give title compound (110 mg). ¹H NMR (400 MHz, CDCl₃) δ 8.45 (s, 2H), 5.15-4.93 (m, 2H), 4.65-4.54 (m, 3H), 4.45 (q, J=4.8 Hz, 1H), 4.15 (d, J=12.6 Hz, 1H), 3.93 (dd, J=3.6, 11.2 Hz, 1H), 3.81-3.50 (m, 6H), 3.44-3.34 (m, 1H), 3.09 (d, J=4.8 Hz, 3H), 2.64 (dd, J=10.6, 13.2 Hz 2H), 1.50 (d, J=1.8 Hz, 6H), 1.25 (s, H). LCMS (ESI): 604.3 [M+H]⁺.

### Example 282. Preparation of Compound E219

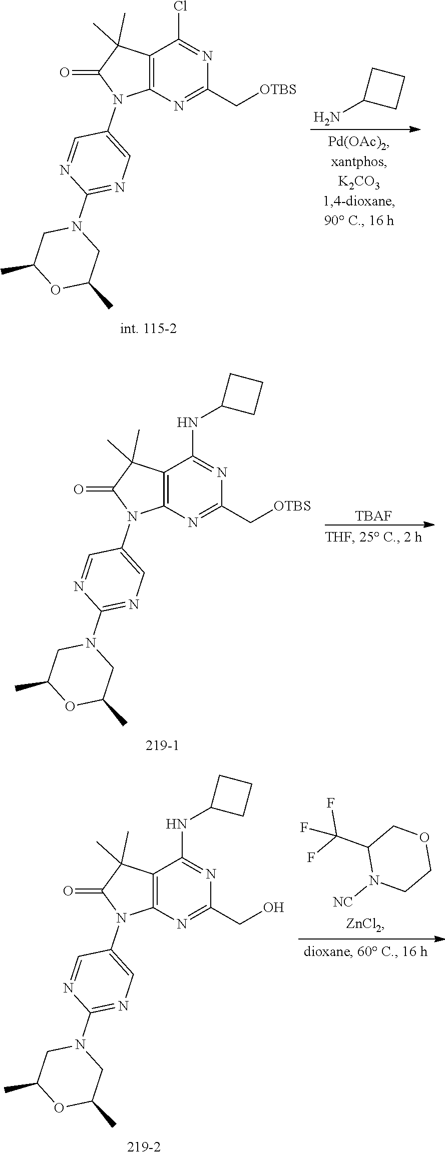 [SMILES: C[C@@H]1CN(c2ncc(N3C(=O)C(C)(C)c4c(Cl)nc(CO[Si](C)(C)C(C)(C)C)nc43)cn2)C[C@H](C)O1 ; NC1CCC1 ; C[C@@H]1CN(c2ncc(N3C(=O)C(C)(C)c4c(NC5CCC5)nc(CO[Si](C)(C)C(C)(C)C)nc43)cn2)C[C@H](C)O1 ; "TBAF" ; C[C@@H]1CN(c2ncc(N3C(=O)C(C)(C)c4c(NC5CCC5)nc(CO)nc43)cn2)C[C@H](C)O1 ; N#CN1CCOCC1C(F)(F)F]

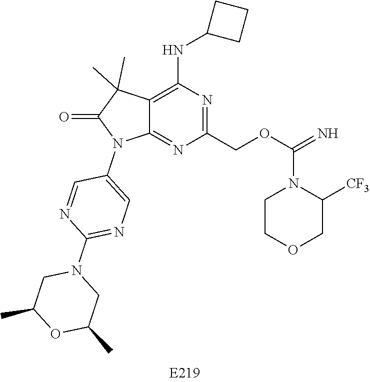 [SMILES: C[C@@H]1CN(c2ncc(N3C(=O)C(C)(C)c4c(NC5CCC5)nc(COC(=N)N5CCOCC5C(F)(F)F)nc43)cn2)C[C@H](C)O1]

1. Synthesis of compound 219-1. To a solution of 2-{[(tert-butyldimethylsilyl)oxy]methyl}-4-chloro-7-{2-[(2R,6S)-2,6-dimethylmorpholin-4-yl]pyrimidin-5-yl}-5,5-dimethyl-5H,6H,7H-pyrrolo[2,3-d]pyrimidin-6-one (300 mg, 0.56 mmol) and cyclobutylamine (120 mg, 1.69 mmol) in 1,4-dioxane (5 mL) were added Pd(OAc)₂ (12 mg, 0.056 mmol), XantPhos (32 mg, 0.056 mmol) and K₂CO₃ (155 mg, 1.13 mmol). The mixture was stirred at 90° C. for 16 hours under N₂. The mixture was filtered and the filtrate was concentrated. The residue was purified by column chromatography to give title compound (80 mg). LCMS (ESI): m/z, 568.5 [M+H]⁺.

2. Synthesis of compound 219-2. To a solution of 2-{[(tert-butyldimethylsilyl)oxy]methyl}-4-(cyclobutylamino)-7-{2-[(2R,6S)-2,6-dimethylmorpholin-4-yl]pyrimidin-5-yl}-5,5-dimethyl-5H,6H,7H-pyrrolo[2,3-d]pyrimidin-6-one (80 mg, 0.14 mmol) in THF (3 mL) was added TBAF (0.42 mL, 1 M in THF). The mixture was stirred at 25° C. for 2 hours. Water was added and extracted with EtOAc. The combined organic phases were washed with brine, dried over anhydrous Na₂SO₄, filtered and concentrated. The residue was purified by prep-TLC to give title compound (40 mg). LCMS (ESI): m/z, 454.5 [M+H]⁺.

3. Synthesis of compound E219. To a solution of 4-(cyclobutylamino)-7-{2-[(2R,6S)-2,6-dimethylmorpholin-4-yl]pyrimidin-5-yl}-2-(hydroxymethyl)-5,5-dimethyl-5H,6H,7H-pyrrolo[2,3-d]pyrimidin-6-one (40 mg, 0.088 mmol) and 3-(trifluoromethyl)morpholine-4-carbonitrile (32 mg, 0.18 mmol) in 1,4-dioxane (5 mL) was added ZnCl₂ (36 mg, 0.26 mmol). The mixture was stirred at 60° C. for 16 hours under N₂. The mixture was filtered and concentrated. The residue was purified by prep-HPLC to yield title compound (12.2 mg). ¹H NMR (400 MHz, CDCl₃) δ 8.43 (s, 2H), 5.16-4.95 (m, 2H), 4.69-4.58 (m, 5H), 4.17 (d, J=12.4 Hz, 1H), 3.99-3.91 (m, 1H), 3.88-3.52 (m, 6H), 3.45-3.41 (m, 1H), 2.64 (dd, J=10.4, 13.2 Hz, 2H), 2.47-2.46 (m, 2H), 1.96-1.73 (m, 6H), 1.52 (d, J=1.2 Hz, 6H), 1.26 (d, J=6.0 Hz, 6H). LCMS (ESI):

### Example 283. Preparation of Compound E220

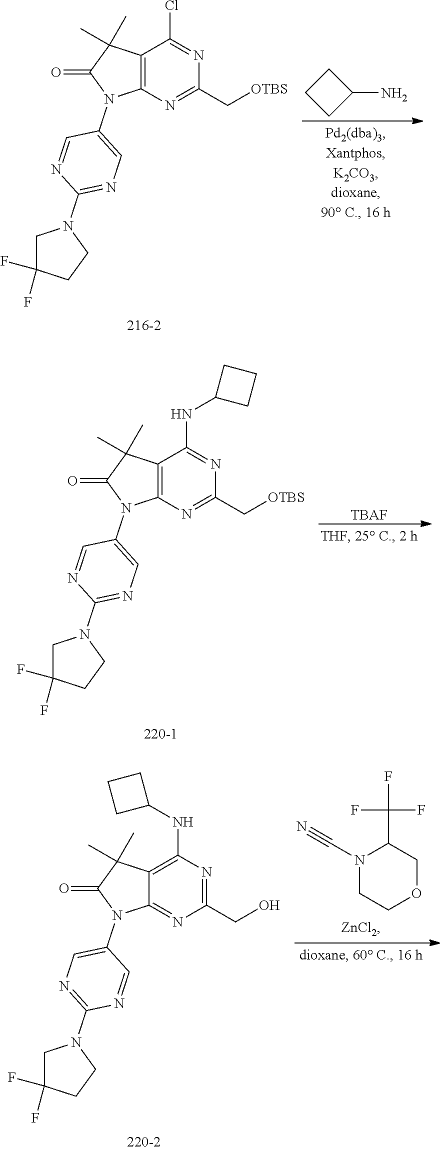 [SMILES: CC1(C)C(=O)N(c2cnc(N3CCC(F)(F)C3)nc2)c2nc(CO[Si](C)(C)C(C)(C)C)nc(Cl)c21 ; NC1CCC1 ; CC1(C)C(=O)N(c2cnc(N3CCC(F)(F)C3)nc2)c2nc(CO[Si](C)(C)C(C)(C)C)nc(NC3CCC3)c21 ; "TBAF" ; CC1(C)C(=O)N(c2cnc(N3CCC(F)(F)C3)nc2)c2nc(CO)nc(NC3CCC3)c21 ; N#CN1CCOCC1C(F)(F)F]

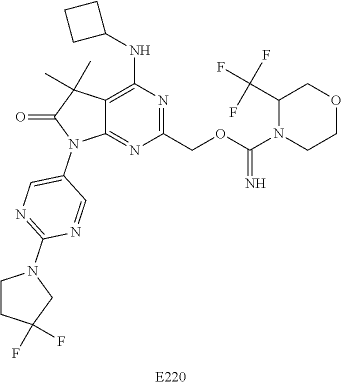 [SMILES: CC1(C)C(=O)N(c2cnc(N3CCC(F)(F)C3)nc2)c2nc(COC(=N)N3CCOCC3C(F)(F)F)nc(NC3CCC3)c21]

1. Synthesis of compound 220-1. A mixture of Cyclobutylamine (203 mg, 2.86-ol), 2-{[(tert-butyldimethylsilyl)oxy)methyl}-4-chloro-7-[2-(3,3-difluoropyrrolidin-1-yl)pyrimidin-5-yl]-5,5-dimethyl-5H,6H,7H-pyrrolo[2,3-d]pyrimidin-6-one (300 mg, 0.57 mmol), K₂CO₃ (158 mg, 1.14 mmol), Pd(OAc)₂ (13 mg, 0.06 mmol) and XantPhos (67 mg, 0.11 mmol) in 1,4-dioxane (5 mL) was stirred at 80° C. for 16 hours under N₂. To the mixture added EA and H₂, the aqueous phase was extracted with BA. The combined organic layers were washed with brine and dried over Na₂SO₄, filtered and concentrated to afford title compound (300 mg crude).

2. Synthesis of compound 220-2. To a solution of 2-(((tert-butyldiinethylsilyl)oxy)methyl)-4-(cyclobutylainino)-7-(2-(3,3-difluoropyrrolidin-1l-yl)pyrimidin-5-yl)-5,5-dimethyl-5,7-dihydro-6H-pyrrolo[2,3-d]pyrimidin-6-one (300 mg, 0.54 mmol) in THF (5 mL) was added TBAF (1.07 mL, 1 M in THF). The reaction was stirred at 25° C. for 1 hour. To the mixture was added BA and H₂O, the aqueous phase was extracted with BA. The combined organic layers were washed with brine and dried over Na₂SO₄, filtered and concentrated. The residue was purified by column chromatography to afford title compound (120 mg. LCMS (ESI): m/z, 446.1 [M+H]⁺.

3. Synthesis of compound E220. A mixture of 4-(cyclobutylamino)-7-(2-(3,3-difluoropyrrolidin-1-yl)pyrimidin-5-yl)-2-(hydroxymethyl)-5,5-dimethyl-5,7-dihydro-6H-pyrrolo[2,3-d]pyrimidin-6-one (120 mg, 0.26 mmol), 3-(trifluoromethyl)morpholine-4-carbonitrile (98 mg, 0.54 mmol) and ZnCl₂ (74 mg, 0.54 mol) in 1,4-dioxane (5 mL) was stirred at 60° C. for 16 hours under N₂. The reaction mixture was filtered and the filter liquid was concentrated. The crude product was purified by prep-HPLC to afford title compound (48 mg).¹H NMR (400 MHz, DMSO-d₆) b 8.46 (s, 2H), 6.84-6.75 (m, 1H), 6.14-6.04 (m, 1H), 5.00-4.82 (m, 2H), 4.73-4.63 (m, 2H), 4.04-3.55 (m, 9H), 3.12-3.09 (m, 1H), 2.61-2.54 (m, 2H), 2.22-2.11 (m, 4H), 1.69-1.57 (m, 2H), 1.47 (s, 6H). LCMS (ESI): m/z, 626.2 [M+H]⁺.

### Example 285. Preparation of Compound F55

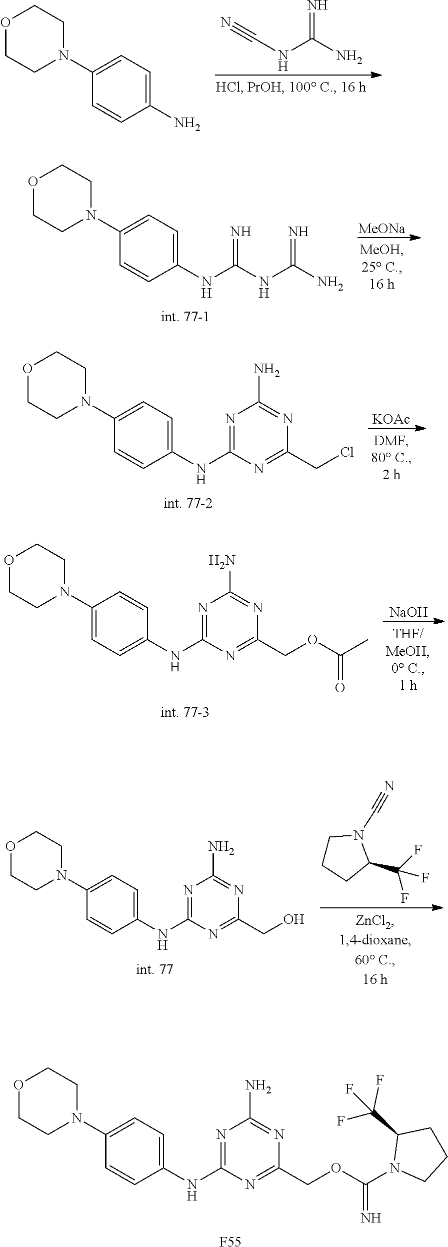

1. Synthesis of compound int. 77-1. To a solution of 4-morpholinoaniline (500 mg, 2.81 mmol) and dicyandiamide (235 mg, 2.81 mmol) in PrOH (7 mL) was added HCl (37%, 112 mg, 3.09 mmol). The reaction mixture was heated up to 100° C. for 16 h. The reaction mixture was cooled to room temperature and filtered. Filter cake was collected and dried to afford 1-carbamimidamido-N-[4-(morpholin-4-yl) phenyl]methanimidamide (221 mg). LCMS (ESI): m/z, 263.1[M+H]⁺.

2. Synthesis of compound int. 77-2. To a solution of 1-carbamimidamido-N-[4-(morpholin-4-yl) phenyl]methanimidamide (221 mg, 0.85 mmol) in MeOH (5 mL) was added MeONa (68 mg, 1.26 mmol) at 0° C., ethyl chloroacetate (154 mg, 1.26 mmol) was followed. The reaction mixture was allowed to warm to 25° C. and stirred for 16 h. The reaction mixture was concentrated under reduced pressure. The residue was triturated with water and ethanol and filtered. The filter cake was collected and dried under reduced pressure to give 6-(chloromethyl)-N²-[4-(morpholin-4-yl) phenyl]-1,3,5-triazine-2,4-diamine (125 mg). LCMS (ESI): m/z, 321.2[M+H]⁺.

3. Synthesis of compound int. 77-3. To a solution of 6-(chloromethyl)-N²-[4-(morpholin-4-yl) phenyl]-1,3,5-triazine-2,4-diamine (125 mg, 0.39 mmol) in DMF (4 mL) was added KOAc (76.49 mg, 0.78 mmol) at 20° C. The resulting mixture was heated up to 80° C. for 2 h. The reaction mixture was quenched with water (10 mL), and extracted with ethyl acetate. The organic phases were washed with water and brine, dried over Na₂SO₄, filtered and concentrated to give (4-amino-6-{[4-(morpholin-4-yl) phenyl]amino}-1,3,5-triazin-2-yl) methyl acetate (100 mg). LCMS (ESI): m/z, 345.2[M+H]⁺.

4. Synthesis of compound int. 77. A solution of (4-amino-6-{[4-(morpholin-4-yl) phenyl]amino}-1,3,5-triazin-2-yl) methyl acetate (100 mg, 0.29 mmol), in THF (3 mL) and MeOH (3 mL) was added NaOH (11.6 mg, 0.29 mmol). The reaction mixture was stirred at 0° C. for 1 h. The mixture was adjusted PH to 7 with 2 M HCl. The resulting mixture was concentrated under reduced pressure. The residue was triturated with water and ethanol and filtered. The filter cake was dried in vacuo to afford (4-amino-6-((4-morpholinophenyl) amino)-1,3,5-triazin-2-yl) methanol (80 mg). LCMS (ESI): m/z, 303.2[M+H]⁺.

5. Synthesis of compound F55. To a solution of (2R)-2-(trifluoromethyl) pyrrolidine-1-carbonitrile (130 mg, 0.79 mmol) and (4-amino-6-{[4-(morpholin-4-yl) phenyl]amino}-1,3,5-triazin-2-yl) methanol (80 mg, 0.27 mmol) in 1,4-dioxane (8 mL) was added ZnCl₂ (108 mg, 0.79 mmol). The mixture was stirred at 60° C. for 16 h under N₂ atmosphere. The reaction mixture was filtered. The residue was purified by Prep-HPLC to yield (4-amino-6-((4-morpholinophenyl) amino)-1,3,5-triazin-2-yl) methyl(R)-2-(trifluoromethyl) pyrrolidine-1-carbimidate (40 mg). ¹H NMR (400 MHz, DMSO-d(₆) δ 9.23 (s, 1H), 7.55 (d, J=8.4 Hz, 2H), 6.96 (s, 2H), 6.84 (d, J=8.4 Hz, 2H), 5.89 (s, 1H), 4.89 (d, J=14.4 Hz, 1H), 4.75 (d, J=14.4 Hz, 1H), 4.64 (s, 1H), 3.87-3.58 (m, 4H), 3.46-3.36 (m, 2H), 3.11-2.84 (m, 4H), 2.13-1.71 (m, 4H). LCMS (ESI): m/z, 467.3[M+H]⁺.

Those skilled in the art reading the present disclosure appreciate that other compounds, e.g., additional compounds in Table C1 and Table C2, can be prepared in accordance with the present disclosure. Among other things, provided compounds can inhibit cell proliferation and are useful for treating various conditions, disorders or diseases including cancer. Certain useful technologies for assessing and confirming properties and activities of provided compounds are presented below as examples.

### Example 286. Provided Technologies can Inhibit Cell Proliferation

Among other things, provided compounds can inhibit cell proliferation. Various technologies can be utilized for assess cell proliferation in accordance with the present disclosure. In some embodiments, a cell viability assay was performed to assess the potency of various compounds towards various cells, e.g., KP4 (pancreatic ductal adenocarcinoma), 786-0 (renal cell carcinoma), HT-1080 (fibrosarcoma), etc.

In one assessment, cells (KP4, 786-0 and HT-1080) were seeded (700 cells/well, 1000 cells/well or 3000 cells/well) in 384-well or 96-well plates and incubated at 37° C./5% CO₂ for 22-24 hours. A series of 8 different concentrations of compound stocks were created by 5-fold serial dilution in DMSO. These compounds were further diluted in culture media and were then added to cells (final concentrations: 100 μM, 20 μM, 4 μM, 0.8 μM, 0.16 μM, 0.0032 μM, 0.00128 μM.). After 72 hours of incubation, 30 μL of CellTiter Glo reagent (Promega) was added to each well and luminescence was measured after 10 minutes using SpectraMax M5/M5e (Molecular Devices). Luminescence from cells treated with DMSO alone was set as background and % of inhibition was calculated as follows:

$$Inhibition \% = ( {Value}_{Background} - {Value}_{sample} ) / {Value}_{Background} ⋆ 100$$

Cell proliferation data for certain compounds are provided in Table 1, Table 2 and Table 3 as examples, wherein “A” indicates an IC₅₀≤100 nM; “B” indicates an IC₅₀ of >100 nM and ≤500 nM; “C” indicates an IC₅₀>500 nM and ≤5 μM; and “D” indicates an IC₅₀>5 uM.

TABLE 1
Inhibition of KP4 Cell Proliferation by Certain Compounds.

| Compound No. | IC₅₀ | Compound No. | IC₅₀ | Compound No. | IC₅₀ |
|---|---|---|---|---|---|
| A1 | B | D10 | C | F24 | C |
| A2 | D | F1 | C | F26 | D |
| D1 | B | F3 | D | F27 | B |
| D2 | A | F4 | A | F31 | B |
| D3 | C | F5 | B | F32 | A |
| D4 | C | F6 | A | F33 | B |
| D5 | B | F7 | B | F34 | A |
| D6 | B | F9 | B | F35 | B |
| D7 | C | F13 | A | F36 | C |
| D8 | C | F23 | C | F38 | D |

TABLE 2
Inhibition of 786-O Cell Proliferation by Certain Compounds.

| Compound No. | IC₅₀ | Compound No. | IC₅₀ | Compound No. | IC₅₀ |
|---|---|---|---|---|---|
| A2 | C | E9 | A | F23 | B |
| A3 | D | E10 | A | F24 | B |
| A4 | C | E11 | C | F25 | C |
| B1 | D | E12 | B | F28 | B |
| B2 | C | E13 | B | F29 | A |
| C1 | D | E14 | B | F31 | B |
| D3 | C | F2 | D | F35 | C |
| D4 | B | F5 | B | F39 | C |
| D5 | B | F6 | B | F40 | B |
| D6 | B | F7 | B | F41 | A |
| D7 | C | F8 | A | F42 | A |
| D8 | B | F9 | A | F43 | B |
| D9 | C | F10 | A | F44 | B |
| D11 | C | F12 | A | F45 | A |
| D12 | B | F13 | A | F46 | A |
| E1 | A | F14 | A | F47 | A |
| E2 | A | F15 | B | F48 | C |
| E3 | B | F16 | C | F49 | C |
| E4 | A | F17 | B | F50 | C |
| E5 | B | F19 | B | F51 | A |
| E6 | B | F20 | B | F52 | D |
| E7 | A | F21 | A | F53 | D |
| E8 | D | F22 | A | F54 | C |
| E15 | A | E88 | A | F55 | B |
| E16 | A | E89 | C | E151 | D |
| E17 | A | E90 | B | E152 | A |
| E18 | A | E91 | A | E153 | A |
| E19 | A | E92 | B | E154 | A |
| E20 | A | E93 | A | E155 | A |
| E21 | A | E94 | A | E156 | C |
| E22 | A | E95 | A | E157 | D |
| E23 | A | E96 | B | E158 | C |
| E27 | C | E97 | B | E159 | B |
| E29 | A | E98 | C | E160 | C |
| E30 | A | E99 | C | E161 | B |
| E31 | A | E100 | B | E162 | C |
| E32 | A | E101 | C | E163 | B |
| E33 | A | E102 | B | E164 | A |
| E34 | A | E103 | B | E165 | D |
| E35 | A | E104 | A | E166 | A |
| E36 | A | E104A | B | E167 | A |
| E37 | A | E104B | A | E168 | A |
| E38 | A | E105 | A | E169 | B |
| E39 | A | E106 | A | E170 | A |
| E41 | B | E107 | A | E171 | A |
| E42 | B | E108 | A | E172 | A |
| E43 | A | E109 | A | E173 | A |
| E44 | A | E110 | B | E174 | A |
| E45 | A | E111 | A | E175 | A |
| E46 | A | E112 | A | E176 | A |
| E47 | B | E113 | A | E177 | A |
| E48 | A | E114 | A | E178 | B |
| E49 | A | E115 | B | E179 | A |
| E50 | A | E116 | B | E180 | D |
| E51 | A | E117 | A | E181 | D |
| E52 | B | E118 | A | E182 | A |
| E53 | A | E119 | A | E183 | B |
| E54 | A | E120 | C | E184 | A |
| E55 | A | E121 | C | E185 | A |
| E56 | A | E122 | C | E186 | A |
| E58 | A | E123 | D | E187 | B |
| E59 | A | E124 | D | E188 | A |
| E60 | A | E125 | C | E189 | B |
| E61 | A | E126 | C | E190 | B |
| E62 | A | E127 | B | E191 | B |
| E63 | A | E128 | B | E192 | B |
| E65 | A | E129 | B | E193 | B |
| E66 | A | E131 | B | E194 | B |
| E67 | A | E132 | B | E195 | B |
| E68 | A | E133 | C | E197 | A |
| E69 | A | E134 | C | E198 | A |
| E70 | A | E135 | C | E199 | C |
| E71 | A | E136 | A | E200 | A |
| E72 | A | E137 | A | E201 | A |
| E73 | A | E138 | B | E202 | C |
| E74 | B | E139 | C | E203 | B |
| E75 | A | E140 | C | E204 | A |
| E76 | B | E141 | B | E205 | A |
| E77 | A | E142 | C | E206 | B |
| E78 | A | E143 | B | E207 | A |
| E79 | B | E144 | C | E208 | A |
| E80 | A | E145 | B | E209 | A |
| E81 | A | E146 | C | E210 | A |
| E82 | A | E147 | D | E211 | A |
| E83 | A | E148 | D | E212 | A |
| E84 | A | E149 | C | E213 | B |
| E85 | A | E150 | B | E214 | A |
| E86 | A | E216 | B | E215 | B |
| E87 | A | E217 | A | E218 | C |

| | | | |
|---|---|---|---|
| E219 | B | E220 | B |

TABLE 3
Inhibition of HT-1080 Cell Proliferation by Certain Compounds.

| Compound No. | IC₅₀ | Compound No. | IC₅₀ | Compound No. | IC₅₀ |
|---|---|---|---|---|---|
| D2 | B | E5 | B | F19 | C |
| D3 | C | F6 | A | F20 | B |
| D12 | C | F9 | B | F22 | B |
| E2 | A | F13 | A | F31 | B |
| E4 | A | F18 | C | | |

Anti-proliferation activity of various compounds was also tested in the presence of an ferroptosis inhibitor, Ferrostatin-1 (2 μM) or Liproxstatin-1 (2 μM). Certain results are provided in Table 4 and Table 5 as examples. “A” indicates an IC50≤100 nM; “B” indicates an IC50 of >100 nM and ≤500 nM; “C” indicates an IC50>500 nM and ≤5 μM; and “D” indicates an IC50>5 μM.

TABLE 4
Inhibition of 786-O Cell Proliferation by Certain
Compounds in the Presence of Ferrostatin-1.

| Compound | IC₅₀ | |
|---|---|---|
| No. | No Ferroptosis Inhibitor | With Ferrostatin-1 |
| D6 | B | D |
| F13 | A | D |

TABLE 5
Inhibition of 786-O Cell Proliferation by Certain
Compounds in the Presence of Liproxstatin-1.

| Compound | IC50 | |
|---|---|---|
| No. | No Ferroptosis Inhibitor | With Liproxstatin-1 |
| D12 | B | D |
| E1 | A | D |
| E2 | A | D |
| F12 | A | D |
| F19 | B | D |
| F20 | B | D |
| F21 | A | D |
| F22 | A | D |
| E87 | A | D |
| E88 | A | D |
| E93 | A | D |
| E94 | A | D |
| E97 | B | D |
| E99 | C | D |
| E100 | B | D |

As demonstrated herein, Ferrostatin-1 and Liproxstatin-1 can significantly reduce or prevent cell proliferation inhibition by various provided compounds.

### Example 93. Provided Compounds Demonstrate Suitable Stability Profiles

Among other things, provides compounds possess properties suitable for various applications. For example, in some embodiments, provided compounds have suitable stability profiles for various applications including therapeutic uses. Various technologies are available in the art for assessing properties, e.g., stability, of compounds and compositions, and can be utilized in accordance with the present disclosure. Certain useful technologies are described herein as examples. In some embodiments, stability was assessed in fresh blood samples, e.g., of human or mouse.

In some embodiments, fresh blood samples were collected from male Balb/c mouse with EDTA-K2 as anti-coagulant. Blood was collected before experiment and stored at 2˜8° C., and equilibrated to room temperature before use. Compounds were dissolved in DMSO at 10 mM concentration. Glibenclamide, used as an internal standard (1S), was prepared in DMSO at 1 mg/mL concentration. An aliquot of 594 μL of blank blood was spiked with 6 μL of working solution (100 μM) to achieve 1 μM of final concentration for test compounds. Solution samples were incubated for 0, 10, 30, 60, 90 and 120 minutes at 37° C., e.g., in a water bath.

An Agilent ZORBAX XDB-C18 (3.5 μm, 50×2.1 mm) was used for HPLC analysis. Mobile phases were 0.1% formic acid in water (mobile phase A), and 0.1% formic acid in acetonitrile (mobile phase B). The injection volume was 10 μL. Mass spectrometer was calibrated and tuned for each test compound. The % remaining of test compound in blood was calculated using the following equation:

$$\% Remaining = 1 \; 00 \times ( PAR at a {T}_{x} / PAR at {T}_{0} ) wherein PAR is the Peak Area Ratio of analyte versus internal standard ( VS ) . {T}_{X} is incubation time .$$

Data for certain compounds are presented in Table 6 as examples.

TABLE 6
Whole Blood Stability of Certain Compounds.

| Compound No. | Species | % Remaining | T_{1/2} (min) |
|---|---|---|---|
| F6 | Human | 84% @ 120 min | 364 |
| | Mouse | 78% @ 120 min | 339 |
| F13 | Human | 57.6% @ 240 min | 321 |
| | Mouse | 74% @ 240 min | 477 |
| E2 | Human | 69.7% @ 240 min | 471 |
| | Mouse | 63.9% @ 240 min | 388 |
| Ref 1ª | Mouse | 18.7% @ 120 min | 47 |
| Ref 2ª | Mouse | 11.7% @ 120 min | 36 |

^{a}Ref 1 and Ref 2 are compound 11 and 12 described in WO 2020/176757, respectively.

As demonstrated herein, among other things, provided technologies can provide improved stability compared to reference technologies, e.g., compounds Ref 1 and Ref 2.

While various embodiments have been described and illustrated herein, those of ordinary skill in the art will readily envision a variety of other means and/or structures for performing the functions and/or obtaining the results and/or one or more of the advantages described in the present disclosure, and each of such variations and/or modifications is deemed to be included. More generally, those skilled in the art will readily appreciate that all parameters, dimensions, materials, and configurations described herein are meant to be example and that the actual parameters, dimensions, materials, and/or configurations may depend upon the specific application or applications for which the teachings of the present disclosure is/are used. Those skilled in the art will recognize, or be able to ascertain using no more than routine experimentation, many equivalents to the embodiments of the present disclosure. It is, therefore, to be understood that the foregoing embodiments are presented by way of example only and that, within the scope of the appended claims and equivalents thereto, claimed technologies may be practiced otherwise than as specifically described and claimed. In addition, any combination of two or more features, systems, articles, materials, kits, and/or methods, if such features, systems, articles, materials, kits, and/or methods are not mutually inconsistent, is included within the scope of the present disclosure.

1. A compound having a structure of formula B-1: 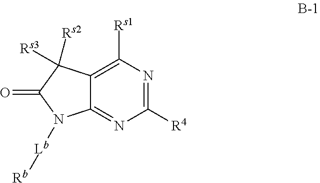
or a salt thereof, wherein:
L^{b} is L;
R^{b} is R″;
R⁴ is or comprises an isourea or isothiourea moiety, or -L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
each R″ is independently hydrogen, halogen, -L-R′, -L-OR′, -L-SR′, -L-C(O)OR′, -L-C(O)SR′, -L-C(O)N(R′)₂, -L-OC(O)N(R′)₂, -L-C(O)R′, -L-N(R′)₂, —CN, —OC(R′)₂COOH, —SC(R′)₂COOH, —N(R′)C(R′)₂COOH, or is an optionally substituted group selected from alkylsulfonyl, arylsulfonyl, carboxylate, ester, ether, amide, carbohydrate, amino acid, acyl, alkyloxy-substituted acyl, alditol, sulfate, sulfonamide, sulfoxide, sulfonate, sulfone, thioalkyl, thioester, and thioether;
each R′ is independently R, —OR, —C(O)R, —C(O)OR, —C(O)N(R)₂, —S(O)R, or —S(O)OR;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
each of R^{s1}, R^{s2} and R^{s3} is independently R″, halogen, —CN, oxo, —NO₂, or an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl.

2. A compound having the structure of formula B: 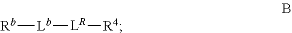
or a salt thereof, wherein:
L^{R} is 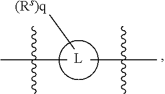
L, —C≡C—, optionally substituted —CH═CH—, —C(O)—, —C(S)—, or —C(NR″)—;
L^{b} is L;
R^{b} is R″;
Ring L is an optionally substituted, 3-20 membered, monocyclic, bicyclic or polycyclic ring having 0-10 heteroatoms;
R⁴ is or comprises an isourea or isothiourea moiety, or -L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w})N(R^{w2})(R^{w3});
each of R^{w1}, R^{w2} and R^{w3} is independently -L-R′ or a detectable moiety;
T is O or S;
each of R^{s} is independently R″, halogen, —CN, oxo, —NO₂, or an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl;
q is 0-10;
each R″ is independently hydrogen, halogen, -L-R′, -L-OR′, -L-SR′, -L-C(O)OR′, -L-C(O)SR′, -L-C(O)N(R′)₂, -L-OC(O)N(R′)₂, -L-C(O)R′, -L-N(R′)₂, —CN, —OC(R′)₂COOH, —SC(R′)₂COOH, —N(R′)C(R′)₂COOH, or is an optionally substituted group selected from alkylsulfonyl, arylsulfonyl, carboxylate, ester, ether, amide, carbohydrate, amino acid, acyl, alkyloxy-substituted acyl, alditol, sulfate, sulfonamide, sulfoxide, sulfonate, sulfone, thioalkyl, thioester, and thioether;
each R′ is independently R, —OR, —C(O)R, —C(O)OR, —C(O)N(R)₂, —S(O)R, or —S(O)OR;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

3. The compound of claim 2, wherein L^{R} is

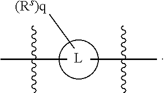

4. The compound of any one of claim 2, wherein:
L^{R} is optionally substituted 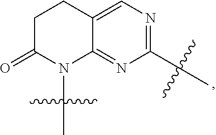 [SMILES: *c1ncc2c(n1)N(*)C(=O)CC2]
wherein the nitrogen atom is boned to L^{b};
L^{R} is optionally substituted 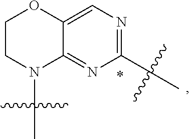 [SMILES: *c1ncc2c(n1)N(*)CCO2]
wherein “*” indicates the atom bonded to R⁴;
L^{R} is optionally substituted 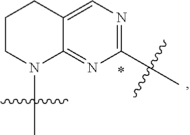 [SMILES: *c1ncc2c(n1)N(*)CCC2]
wherein “*” indicates the atom bonded to R⁴;
L^{R} is optionally substituted 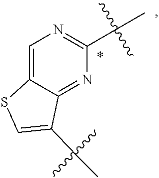 [SMILES: *c1ncc2scc(*)c2n1]
wherein “*” indicates the atom bonded to R⁴;
L^{R} is optionally substituted 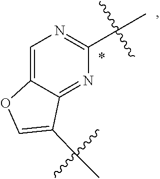 [SMILES: *c1ncc2occ(*)c2n1]
wherein “*” indicates the atom bonded to R⁴;
L^{R} is optionally substituted 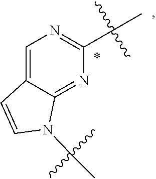 [SMILES: *c1ncc2ccn(*)c2n1]
wherein “*” indicates the atom bonded to R⁴;
L^{R} is optionally substitute 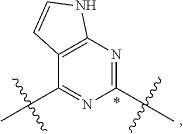 [SMILES: *c1nc(*)c2cc[nH]c2n1]
wherein “*” indicates the atom bonded to R⁴;
L^{R} is optionally substituted 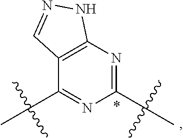 [SMILES: *c1nc(*)c2cn[nH]c2n1]
wherein “*” indicates the atom bonded to R⁴;
L^{R} is optionally substituted 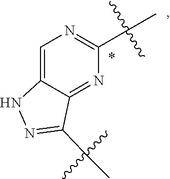 [SMILES: *c1ncc2[nH]nc(*)c2n1]
wherein indicates the atom bonded to R⁴;
L^{R} is optionally substituted 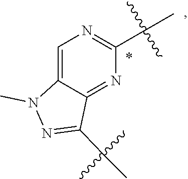 [SMILES: *c1ncc2c(n1)c(*)nn2C]
wherein indicates the atom bonded to R⁴;
L^{R} is 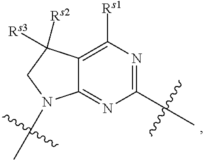
wherein the nitrogen atom is boned to L^{b}, wherein each of R^{s1}, R^{s2} and R^{s3} is independently R^{s}; or
L^{R} is 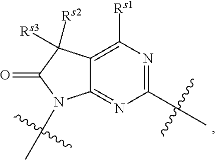
wherein the nitrogen atom is boned to L^{b}, wherein each of R^{s1}, R^{s2} and R^{s3} is independently R^{s}.

5. The compound of claim 1 or 2, wherein:
the compound has a structure of B-1a, B-1b, B-1c, or B-1d: 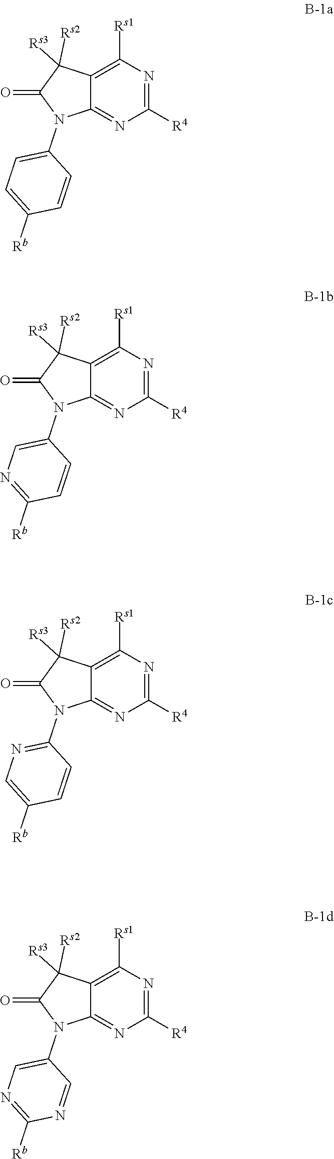
or a salt thereof;
the compound has a structure of B-2a, B-2b, B-2c, B-2d, or B-2e: 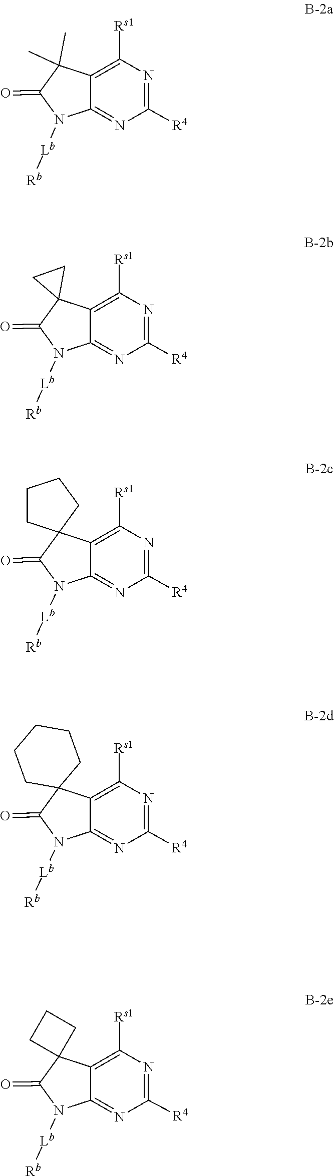
or a salt thereof;
the compound has a structure of B-3a, B-3b, B-3c, or B-3d: 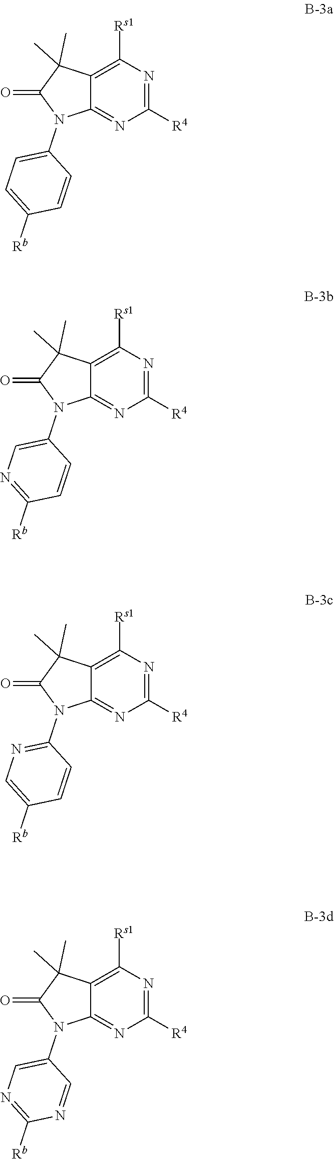
or a salt thereof,
the compound has a structure of B-4a, B-4b, B-4c, or B-4d: 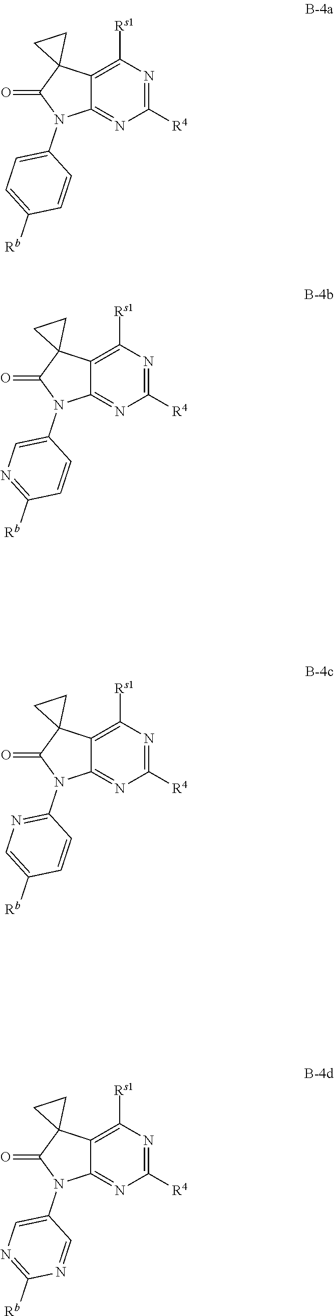
or a salt thereof,
the compound has a structure of B-5a, B-5b, B-5c, or B-5d: 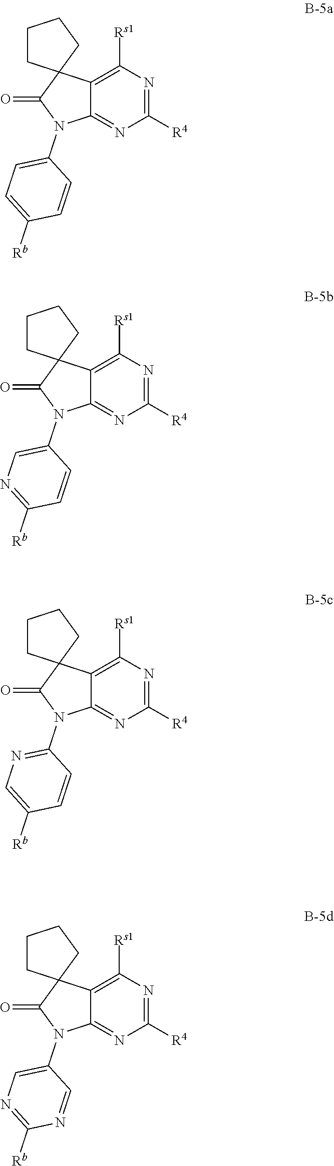
or a salt thereof,
wherein the compound has a structure of B-6a, B-6b, B-6c, or B-6d: 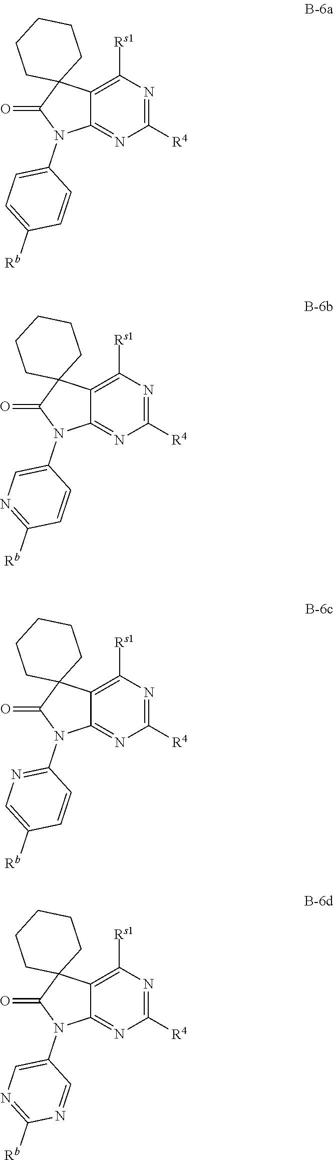
or a salt thereof; or
wherein the compound has a structure of B-7a, B-7b, B-7c, or B-7d: 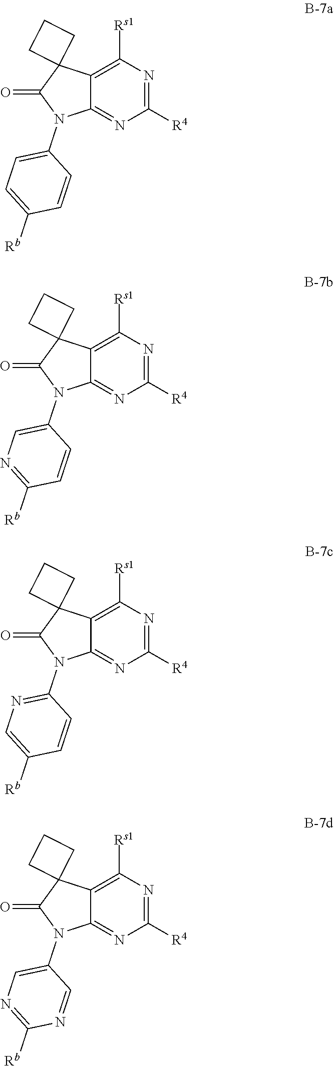
or a salt thereof.

6. The compound of any one of claims 1-5, wherein R^{s1} is —N(R′)₂, —OR, optionally substituted C₁₋₆ aliphatic (e.g., —CF₃ or CHF₂), halogen.

7. The compound of any one of claims 1-5, wherein R^{s1} is —H, —NH₂, —Cl, —NHCH₃, —CHF₂, —CF₃, —NHCD₃, —NHCH₂CH₃, —NHCH₂CH₂CH₃, —NHCH(CH₃)₂, —N(CH₃)₂, —NHCH₂CF₃, —NHCH₂CHF₂, —NHCH₂CH═CH₂, —OH, —OCH₃,

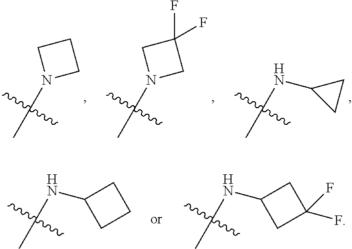 [SMILES: *N1CCC1 ; *N1CC(F)(F)C1 ; *NC1CC1 ; *NC1CCC1 ; "or" ; *NC1CC(F)(F)C1]

8. The compound of any one of claims 1-7, wherein each of R^{s2} and R^{s3} is independently —H or optionally substituted C₁₋₆ aliphatic.

9. The compound of claims 1-7, wherein R^{s2} and R^{S3} are taken together with the carbon atom to which they are attached to form an optionally substituted 3-10 membered saturated or partially unsaturated ring having 0-4 heteroatoms; an optionally substituted 3-6 membered saturated or partially unsaturated carbocyclyl ring; or an optionally substituted 3-6 membered saturated or partially unsaturated ring having 1-4 heteroatoms.

10. The compound of claim 9, wherein:
R^{s2} and R^{s3} are taken together with the carbon atom to which they are attached to form an optionally substituted cyclopropyl ring;
R^{s2} and R^{s3} are taken together with the carbon atom to which they are attached to form a cyclopropyl ring.
wherein R^{s2} and R^{s3} are taken together with the carbon atom to which they are attached to form an optionally substituted cyclobutyl ring;
R^{s2} and R^{s3} are taken together with the carbon atom to which they are attached to form a cyclobutyl ring;
R^{s2} and R^{s3} are taken together with the carbon atom to which they are attached to form an optionally substituted cyclopentyl ring;
R^{s2} and R^{s3} are taken together with the carbon atom to which they are attached to form a cyclopentyl ring;
R^{s2} and R^{S3} are taken together with the carbon atom to which they are attached to form an optionally substituted cyclohexyl ring;
R^{s2} and R^{S3} are taken together with the carbon atom to which they are attached to form a cyclohexyl ring;
R^{s2} and R^{S3} are taken together with the carbon atom to which they are attached to form optionally substituted 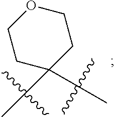 [SMILES: *C1(*)CCOCC1]
R^{s2} and R^{s3} are taken together with the carbon atom to which they are attached to form 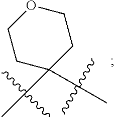 [SMILES: *C1(*)CCOCC1]
R^{s2} and R^{s3} are taken together with the carbon atom to which they are attached to form optionally substituted 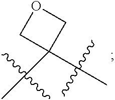 [SMILES: *C1(*)COC1]
or
R^{s2} and R^{s3} are taken together with the carbon atom to which they are attached to form

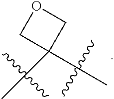 [SMILES: *C1(*)COC1]

11. The compound of claim 2, wherein L^{R} is 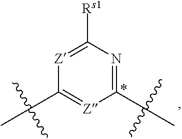
wherein:
“*” indicates the atom bonded to R⁴;
R^{s1} is R^{s};
Z′ is —N═ or —C(R^{s4})═;
Z″ is —N═ or —C(R^{s5})=; and
each of R^{s4} and R^{s5} is independently R^{s}.

12. The compound of claim 11, wherein:
L^{R} is 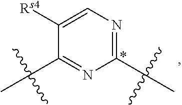
wherein “*” indicates the atom bonded to R⁴; or
L^{R} is 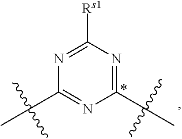
wherein “*” indicates the atom bonded to R⁴.

13. The compound of any one claims 11-12, wherein:
R^{s1} is —N(R′)₂ or —OR; or
R^{s1} is —H, —NH₂ or —NHCH₃.

14. The compound of any one of claims 11-13, wherein R^{s4} is —H, -halogen, —CN, —N(R′)₂ (e.g., —NH₂), or an optionally substituted group selected from C₁₋₁₀ aliphatic, C₁₋₁₀ heteroaliphatic having 1-5 heteroatoms, 6-10membered aryl, 5-10 membered heteroaryl having 1-4 heteroatoms, and 3-10 membered heterocyclyl having 1-5 heteroatoms.

15. The compound of claim 11 or 12, wherein R^{s4} is optionally substituted —OCH(CH₃)₂.

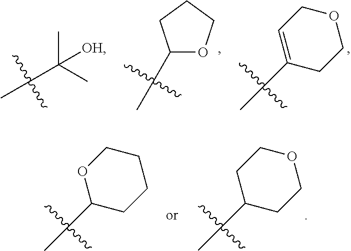

16. The compound of any one of claims 11-15, wherein R^{s5} is —H, -halogen (e.g., —F), —N(R′)₂ (e.g., —NH₂ or —NHCH₃), —OR (e.g., or —OH or —OCH₃) or an optionally substituted group selected from C₁₋₁₀ aliphatic, C₁₋₁₀ heteroaliphatic having 1-5 heteroatoms, C₅₋₈ aryl, 5-8 membered heteroaryl having 1-4 heteroatoms, and 3-10 membered heterocyclyl having 1-5 heteroatoms.

17. The compound of any one of claims 11-16, wherein:
L^{R} is optionally substituted 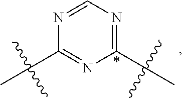 [SMILES: *c1ncnc(*)n1]
wherein “*” indicates the atom bonded to R⁴;
L^{R} is optionally substituted 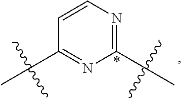 [SMILES: *c1ccnc(*)n1]
wherein “*” indicates the atom bonded to R⁴;
L^{R} is optionally substituted 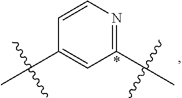 [SMILES: *c1ccnc(*)c1]
wherein “*” indicates the atom bonded to R⁴;
L^{R} is optionally substituted 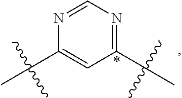 [SMILES: *c1cc(*)ncn1]
wherein “*” indicates the atom bonded to R⁴;
L^{R} is optionally substituted 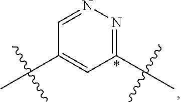 [SMILES: *c1cnnc(*)c1]
wherein “*” indicates the atom bonded to R⁴;
L^{R} is optionally substituted 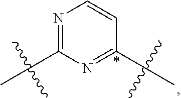 [SMILES: *c1ccnc(*)n1]
wherein “*” indicates the atom bonded to R⁴; or
L^{R} is optionally substituted 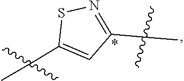 [SMILES: *c1cc(*)sn1]
wherein “*” indicates the atom bonded to R⁴.

18. The compound of any one of the preceding claims, wherein the compound is in a tautomeric form comprising ═O bonded to a ring, or in another tautomeric form the ═O exists as —OH (e.g., wherein the —OH is R^{s1}).

19. The compound of claim 2, wherein L^{R} is a covalent bond, optionally substituted C₁₋₆ alkylene, optionally substituted methylene, optionally substituted C₂₋₆ alkenylene, optionally substituted C₂₋₆ alkynylene, optionally substituted C₃₋₁₀ cycloalkylene, optionally substituted C₆₋₁₄ arylene, optionally substituted C₇₋₁₅ aralkylene, optionally substituted 5-14 membered heteroarylene having 1-5 heteroatoms, or L^{R} is optionally substituted 3-14 membered, heterocyclylene having 1-5 heteroatoms.

20. The compounds of any one of the preceding claims, wherein L^{b} is -L^{b1}-L^{b2}-L^{b3}-L^{b4}-L^{b5}-, wherein each of L^{b1}, L^{b2}, L^{b3}, L^{b4} and L^{b5} is independently L′, wherein each L′ is independently a covalent bond, or an optionally substituted bivalent C₁₋₂ aliphatic or heteroaliphatic having 1-2 heteroatoms, wherein one or more methylene units are optionally and independently replaced with —C(R′)₂—, -Cy-, —O—, —S—, —N(R′)—, —C(O)—, —C(S)—, C(NR′)—, C(O)N(R′)—, O—, —S—, N(R′)—, —C(O)—, —C(S)—, —C(NR′)—N(R′)C(O)N(R′)—, —N(R′)C(O)O—, —S(O)—, —S(O)₂—, —S(O)₂N(R′)—, —C(O)S—, or —C(O)O—, and wherein L^{b1} is bonded to Ring L.

21. The compound of any one the preceding claims, wherein L^{b} is -Cy-.

22. The compound of claim 21, wherein L^{b} is optionally substituted phenyl; or a bivalent optionally substituted 5- or 6-membered heteroaryl ring having 0-4 (e.g., 1-4, 0, 1, 2, 3, 4, etc.) heteroatoms.

23. The compound of claim 21, wherein L^{b} is optionally substituted phenyl, pyridinyl, pyridazinyl, pyrimidinyl, or thiazolyl.

24. The compound of claim 21, wherein L^{b} is phenyl, pyridinyl, pyridazinyl, pyrimidinyl, thiazolyl optionally substituted with halogen (e.g., (—F, —Cl, —Br, or —I) or —OR^{∘} wherein R^{∘} is C₁₋₆ aliphatic (e.g., methyl, ethyl, n-propyl, s-propyl, or isopropyl).

25. The compound of claim 21, wherein L^{b} is 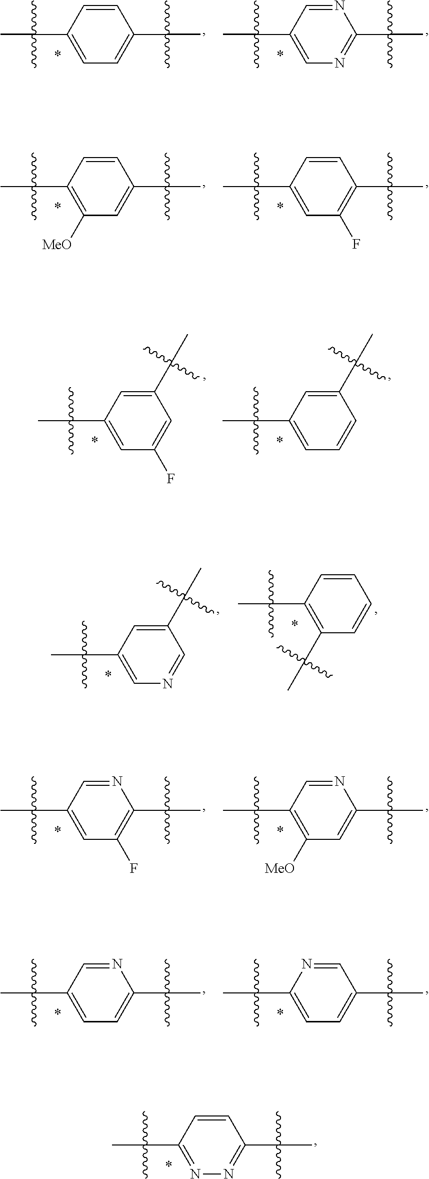
wherein “*” represents the point of attachment to L^{R}.

26. The compound of claim 21, wherein L^{b} is a bivalent optionally substituted 6- to 12-membered heterocyclyl ring having 1-4 (e.g., 0, 1, 2, 3, or 4) heteroatoms; L^{b} is a bivalent optionally substituted 10- to 15-membered tricyclic heterocyclyl ring having 1-4 (e.g., 0, 1, 2, 3, or 4) heteroatoms; L^{b} is a bivalent optionally substituted 6- to 12-membered heteroaryl ring having 1-4 (e.g., 0, 1, 2, 3, or 4) heteroatoms

27. The compound of claim 21, wherein L^{b} is a bivalent optionally substituted 2,3-dihydrobenzofuranyl, chromanyl, or spiro[chromane-4,1′-cyclopropanyl.

28. The compound of claim 21, wherein L^{b} is a bivalent 2,3-dihydrobenzofuranyl, chromanyl, or spiro[chromane-4,1′-cyclopropanyl, optionally substituted with C₁₋₆ aliphatic (e.g., methyl, ethyl, n-propyl, s-propyl, or isopropyl) or halogen (e.g., —F, —Cl, —Br, —I).

29. The compound of claim 21, wherein:
L^{b} is 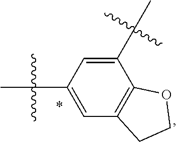 [SMILES: *c1cc(*)c2c(c1)CCO2]
wherein “*” represents the point of attachment to L^{R};
L^{b} is a bivalent optionally substituted chromanyl;
or
R^{b} is —H, and L^{b} is 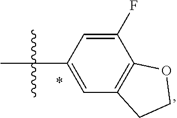 [SMILES: *c1cc(F)c2c(c1)CCO2]
wherein “*” represents the point of attachment to L^{R};
R^{b} is —H, and L^{b} is 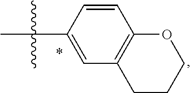 [SMILES: *c1ccc2c(c1)CCCO2]
wherein “*” represents the point of attachment to L^{R};
R^{b} is —H, and L^{b} is 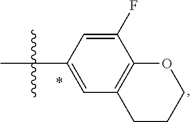 [SMILES: *c1cc(F)c2c(c1)CCCO2]
wherein “*” represents the point of attachment to L^{R};
R^{b} is —H, and L^{b} is

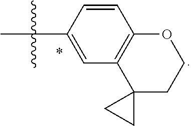 [SMILES: *c1ccc2c(c1)C1(CCO2)CC1]

30. The compound of claim 21, wherein L^{b} is a bivalent optionally substituted 6- to 12-membered heteroaryl ring having 1-4 (e.g., 0, 1, 2, 3, or 4) heteroatoms.

31. The compound of claim 21, wherein L^{b} is a bivalent optionally substituted imidazo[1,5-a]pyridinyl, indazolyl, benzo[d]imidazolyl, or pyrazolyl[1,5-a]pyrimidinyl

32. The compound of claim 21, wherein L^{b} is imidazo[1,5-a]pyridinyl, L^{b} is a bivalent imidazo[1,5-a]pyridinyl, indazolyl, benzo[d]imidazolyl, or pyrazolyl[1,5-a]pyrimidinyl, optionally substituted with C₁₋₆ aliphatic (e.g., methyl, ethyl, n-propyl, s-propyl, or isopropyl) or halogen (e.g., —F, —Cl, —Br, —I).

33. The compound of claim 21, wherein L^{b} is 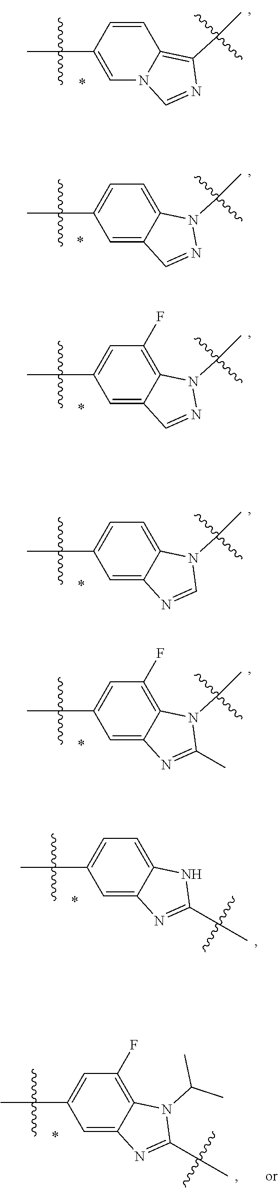 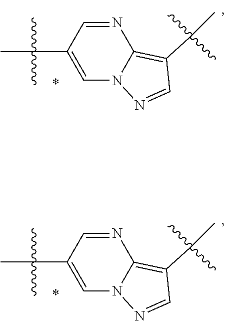 [SMILES: *c1cnc2c(*)cnn2c1 ; *c1cnc2c(*)cnn2c1]
wherein “*” represents the point of attachment to L^{R}.

34. The compound of claim 21, wherein L^{b} is *—N(R′)-Cy-, wherein “*” represents the point of attachment to L^{R}.

35. The compound of claim 21, wherein L^{b} is 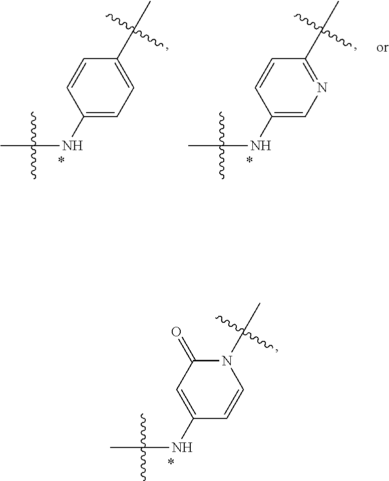 [SMILES: *Nc1ccc(*)cc1 ; *Nc1ccc(*)nc1 ; *Nc1ccn(*)c(=O)c1]
wherein “*” represents the point of attachment to L^{R}.

36. The compound of claim 21, wherein L^{b} is 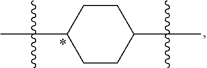 [SMILES: *C1CCC(*)CC1]
wherein “*” represents the point of attachment to L^{R}.

37. The compound of any one of the preceding claims, wherein R^{b} is not hydrogen.

38. The compound of any one of the preceding claims, wherein R^{b} is hydrogen, optionally substituted C₁₋₂₀ aliphatic (e.g., methyl, (e.g., —CF₃), ethyl, isopropyl, —C₀₋₄alkyl-C≡CH, or propargyl), optionally substituted C₃₋₂₀ cycloaliphatic, (e.g., cyclopropyl, cyclopentyl, or cyclohexyl), optionally substituted C₁₋₂₀ heteroaliphatic having 1-10 heteroatoms, —CN, —OH, —OCH₃, —OCH(CH₂CH₃)₂, —OCH₂CH₂CH₂CH₃, —OCH2CH2CH2CH2CH2CH3, —N(R′)₂ (e.g., —N(CH₂CH₃)₂), sulfone (e.g., —S(O₂)CH₃) optionally substituted C₆₋₂₀ aryl (e.g., phenyl or naphthyl), optionally substituted 5-20 membered heteroaryl (e.g., pyridyl), optionally substituted 3-20 membered heterocyclyl having 1-10 heteroatoms (e.g., tetrahydro-2H-pyranyl, morpholinyl, 2-oxa-5-azabicyclo[2.2.1]heptanyl, or 3-oxa-8-azabicyclo[3.2.1]octanyl).

39. The compound of any one of claims 1-38, wherein R^{b} is optionally substituted tetrahydro-2H-pyranyl, morpholinyl, 3,6-dihydro-2H-pyranyl, azetidinyl, pyrrolidinyl, piperidinyl, silinanyl, 2-oxa-5-azabicyclo[2.2.1]heptanyl, 3-oxa-8-azabicyclo[3.2.1]octanyl.

40. The compound of any one of claims 1-38, wherein R^{b} is 4-tetrahydro-2H-pyranyl,

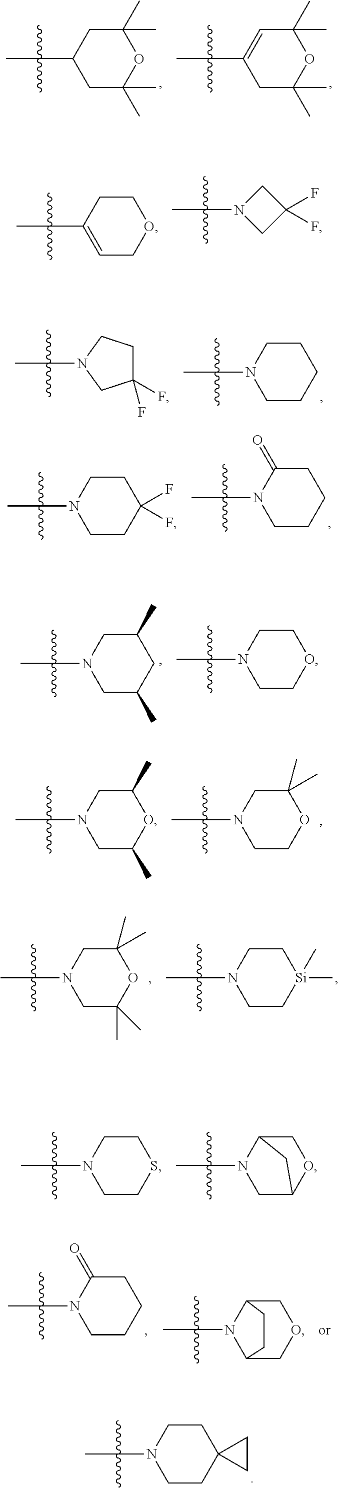

41. The compound of any one of claims 1-38, wherein R^{b} is —O—R′, wherein R′ is optionally substituted 3- to 8-membered saturated or partially unsaturated carbocyclyl; optionally substituted phenyl; or optionally substituted 3- to 7-membered heterocyclyl comprising 1-3 heteroatoms selected from nitrogen, oxygen, or sulfur.

42. The compound of any one of claims 1-38, wherein R^{b} is

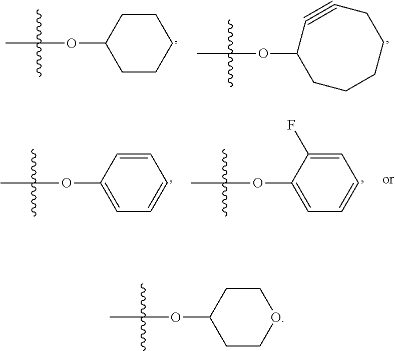 [SMILES: *OC1CCCCC1 ; *OC1C#CCCCCC1 ; *Oc1ccccc1 ; *Oc1ccccc1F ; *OC1CCOCC1]

43. A compound having the structure of formula A′: 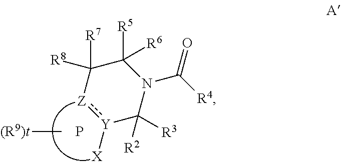
or a salt thereof, wherein:
Ring P is an optionally substituted 5-20 membered, monocyclic, bicyclic or polycyclic ring having 0-5 heteroatoms; [symbol]
is a single bond or double bond;
each of Y and Z is independently C or N;
each of R² and R³ is independently R″ or 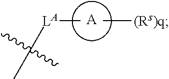
R⁴ is or comprises an isourea or isothiourea moiety, or -L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2}(R^{w3});
each of R^{w1}, R^{w2} and R^{w3} is independently -L-R′ or a detectable moiety;
T is O or S;
each of R^{s} and R⁶ is independently R″ or —C(O)OR″;
each of R⁷ and R^{s} is independently R″;
Ring A is an optionally substituted, 3-20 membered, monocyclic, bicyclic or polycyclic ring having 0-10 heteroatoms;
L^{A} is L;
each of R⁹ and R⁸ is independently R″, halogen, —CN, oxo, —NO₂, or an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl;
each of p and q is independently 0-10;
each R″ is independently hydrogen, halogen, -L-R′, -L-OR′, -L-SR′, -L-C(O)OR′, -L-C(O)SR′, -L-C(O)N(R′)₂, -L-OC(O)N(R′)₂, -L-C(O)R′, -L-N(R′)₂, —CN, —OC(R′)₂COOH, —SC(R′)₂COOH, —N(R′)C(R′)₂COOH, or is an optionally substituted group selected from alkylsulfonyl, arylsulfonyl, carboxylate, ester, ether, amide, carbohydrate, amino acid, acyl, alkyloxy-substituted acyl, alditol, sulfate, sulfonamide, sulfoxide, sulfonate, sulfone, thioalkyl, thioester, and thioether;
each R′ is independently R, —OR, —C(O)R, —C(O)OR, —C(O)N(R)₂, —S(O)R, or —S(O)OR;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.
X is —O—, —S—, —C(R′)═, —N(R′)—, or optionally substituted —CH═, —CH═CH—, or —NH—;
t is 0-9; and
R¹ is R⁹;
or
a compound having the structure of formula C: 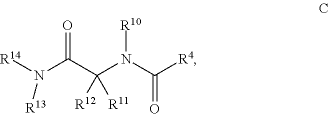
or a salt thereof, wherein:
R⁴ is or comprises an isourea or isothiourea moiety, or -L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
each of R¹⁰, R^{D}, R¹², R¹³, and R¹⁴ is independently R″;
each R″ is independently hydrogen, halogen, -L-R′, -L-OR′, -L-SR′, -L-C(O)OR′, -L-C(O)SR′, -L-C(O)N(R′)₂, -L-OC(O)N(R′)₂, -L-C(O)R′, -L-N(R′)₂, —CN, —OC(R′)₂COOH, —SC(R′)₂COOH, or —N(R′)C(R′)₂COOH;
each R′ is independently R, —OR, —C(O)R, —C(O)OR, —C(O)N(R)₂, —S(O)R, or —S(O)OR;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R is independently halogen, —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms;
or
a compound having the structure of formula D: 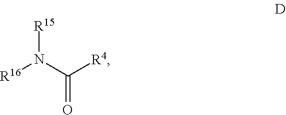
or a salt thereof, wherein:
each of R¹⁵ and R¹⁶ is independently -L^{w}-R^{w};
R⁴ is or comprises an isourea or isothiourea moiety, or -L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R′ is independently R, —OR, —C(O)R, —C(O)OR, —C(O)N(R)₂, —S(O)R, or —S(O)OR;
-Cy- is independently an optionally substituted, 3-20 membered, monocyclic, bicyclic or polycyclic ring having 0-10 heteroatoms;
each R is independently halogen, —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms;
or
a compound having the structure of formula E: 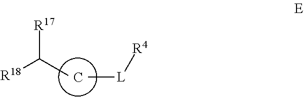
or a salt thereof, wherein:
R⁴ is or comprises an isourea or isothiourea moiety, or -L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
each of R¹⁷ and R¹⁸ is independently an optionally substituted 5-20 membered, monocyclic, bicyclic or polycyclic aromatic ring having 0-5 heteroatoms;
Ring C is an optionally substituted 3-20 membered, monocyclic, bicyclic or polycyclic ring having 0-6 heteroatoms.
L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R′ is independently R, —OR, —C(O)R, —C(O)OR, —C(O)N(R)₂, —S(O)R, or —S(O)OR;
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms;
or
a compound having the structure of formula F: 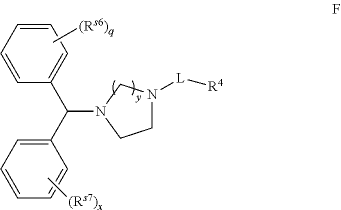
or a salt thereof, wherein:
R⁴ is or comprises an isourea or isothiourea moiety, or -L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
each of R^{s6}, R^{s7} is independently R^{s};
each of R^{s} is independently R″, halogen, —CN, oxo, —NO₂, or an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl;
each R″ is independently hydrogen, halogen, -L-R′, -L-OR′, -L-SR′, -L-C(O)OR′, -L-C(O)SR′, -L-C(O)N(R′)₂, -L-OC(O)N(R′)₂, -L-C(O)R′, -L-N(R′)₂, —CN, —OC(R′)₂COOH, —SC(R′)₂COOH, —N(R′)C(R′)₂COOH, or is an optionally substituted group selected from alkylsulfonyl, arylsulfonyl, carboxylate, ester, ether, amide, carbohydrate, amino acid, acyl, alkyloxy-substituted acyl, alditol, sulfate, sulfonamide, sulfoxide, sulfonate, sulfone, thioalkyl, thioester, and thioether;
each of q, x and y is independently 0-4;
L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, C(O), C(S), C(NR′), —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R′ is independently R, —OR, —C(O)R, —C(O)OR, —C(O)N(R)₂, —S(O)R, or —S(O)OR;
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms;
or
a compound having the structure of formula G: 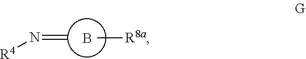
or a salt thereof, wherein:
Ring B is an optionally substituted, 3-20 membered, monocyclic, bicyclic or polycyclic ring having 0-10 heteroatoms;
R⁴ is or comprises an isourea or isothiourea moiety, or -L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
R^{8a} is R^{m}, or 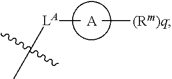
L^{A} is L;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
Ring A is an optionally substituted, 3-20 membered, monocyclic, bicyclic or polycyclic ring having 0-10 heteroatoms;
each R^{m} is independently R^{s};
each of R^{s} is independently R″, halogen, —CN, oxo, —NO₂, or an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl;
each R″ is independently hydrogen, halogen, -L-R′, -L-OR′, -L-SR′, -L-C(O)OR′, -L-C(O)SR′, -L-C(O)N(R′)₂, -L-OC(O)N(R′)₂, -L-C(O)R′, -L-N(R′)₂, —CN, —OC(R′)₂COOH, —SC(R′)₂COOH, —N(R′)C(R′)₂COOH, or is an optionally substituted group selected from alkylsulfonyl, arylsulfonyl, carboxylate, ester, ether, amide, carbohydrate, amino acid, acyl, alkyloxy-substituted acyl, alditol, sulfate, sulfonamide, sulfoxide, sulfonate, sulfone, thioalkyl, thioester, and thioether;
q is independently 0-10;
each R′ is independently R, —OR, —C(O)R, —C(O)OR, —C(O)N(R)₂, —S(O)R, or —S(O)OR;
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms; or
a compound having the structure of formula H: 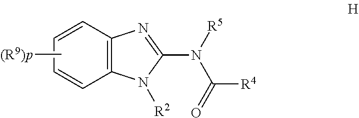
or a salt thereof, wherein:
R⁴ is or comprises an isourea or isothiourea moiety, or -L^{w}-R^{w};
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
R⁵ is independently R″ or —C(O)OR″;
R² is independently R″ or 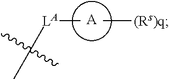
L^{A} is L;
Ring A is an optionally substituted, 3-20 membered, monocyclic, bicyclic or polycyclic ring having 0-10 heteroatoms;
each of R⁹ and R^{s} is independently R″, halogen, —CN, oxo, —NO₂, or an optionally substituted group selected from acyl, acylamino, hydroxy, amino acid, amine, amide, carbamate, ester, ether, carboxylic acid, thio, thioalkyl, thioester, thioether, sulfate, sulfonamide, sulfoxide, sulfonate, sulfone, alkylsulfonyl, and arylsulfonyl;
each of p and q is independently 0-10;
each R″ is independently hydrogen, halogen, -L-R′, -L-OR′, -L-SR′, -L-C(O)OR′, -L-C(O)SR′, -L-C(O)N(R′)₂, -L-OC(O)N(R′)₂, -L-C(O)R′, -L-N(R′)₂, —CN, —OC(R′)₂COOH, —SC(R′)₂COOH, —N(R′)C(R′)₂COOH, or is an optionally substituted group selected from alkylsulfonyl, arylsulfonyl, carboxylate, ester, ether, amide, carbohydrate, amino acid, acyl, alkyloxy-substituted acyl, alditol, sulfate, sulfonamide, sulfoxide, sulfonate, sulfone, thioalkyl, thioester, and thioether;
each R′ is independently R, —OR, —C(O)R, —C(O)OR, —C(O)N(R)₂, —S(O)R, or —S(O)OR;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

44. The compound of any one of the preceding claims, wherein R⁴ is -L^{w}-R^{w}, wherein:
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{w} is -T-C(═NR^{w1})N(R^{w2})(R^{w3});
each of R^{w1}, R^{w2} and R^{w3} is independently -L-R′ or a detectable moiety;
T is O or S;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R′ is independently R, —OR, —C(O)R, —C(O)OR, —C(O)N(R)₂, —S(O)R, or —S(O)OR; and
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

45. The compound of any one of the preceding claims, wherein R⁴ is -L^{w}-R^{wh}, wherein:
L^{w} is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
R^{wh} is -T-H;
T is O or S;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R′ is independently R, —OR, —C(O)R, —C(O)OR, —C(O)N(R)₂, —S(O)R, or —S(O)OR; and
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

46. The compound of any one of the preceding embodiments, wherein L^{w} is a covalent bond, optionally substituted —CH₂—, —CHD-, —CD₂-, mono-substituted —CH₂—, —CH(CH₃)—, 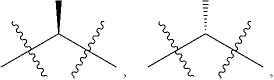
—CHD-, mono-substituted -CD₂-, —C(O)—CH₂—, optionally substituted —C(S)—CH₂—, —C(N(R′))—CH₂— wherein the —CH₂— is optionally substituted, optionally substituted —C(O)—CHD-, optionally substituted —C(S)—CHD-, —C(N(R′))—CHD- wherein the —CHD- is optionally substituted, optionally substituted —C(O)—CD₂-, optionally substituted —C(S)—CD₂-, —C(N(R′))—CD₂- wherein the —CD₂- is optionally substituted.

47. The compound of any one of the preceding claims, wherein T is O.

48. The compound of any one of claims 1-46, wherein T is S.

49. The compound of any one of the preceding embodiments, wherein R^{w1} is -L-R′.

50. The compound of any one of the preceding embodiments, wherein R^{w1} is H, optionally substituted C₁₋₁₀ aliphatic, optionally substituted C₁₋₁₀ alkyl, methyl, ethyl, isopropyl, optionally substituted C₃₋₁₀ cycloalkyl, cyclohexyl, optionally substituted C₁₋₁₀ heteroaliphatic having 1-3 heteroatoms, optionally substituted C₆₋₁₄ aryl, optionally substituted phenyl, optionally substituted 5-10 membered heteroaryl having 1-4 heteroatoms, optionally substituted 3-10 membered heterocyclyl having 1-4 heteroatoms, or a detectable moiety.

51. The compound of any one of the preceding claims, wherein R^{w2} is -L-R′. The compound of any one of the preceding embodiments, wherein R^{w2} is H, optionally substituted C₁₋₁₀ aliphatic, optionally substituted C₁₋₁₀ alkyl, optionally substituted C₁₋₆ haloalkyl, methyl, ethyl, isopropyl, —C(CH₂)₂—C≡C—CH₃, —CH₂—C≡CH, optionally substituted C₃₋₁₀ cycloalkyl, cyclopropyl, cyclohexyl, optionally substituted adamantly, 1-adamantyl, —CH₂—CF₃, optionally substituted C₁₋₁₀ heteroaliphatic having 1-3 heteroatoms, optionally substituted C₆₋₁₄ aryl, optionally substituted phenyl, optionally substituted 5-10 membered heteroaryl having 1-4 heteroatoms, optionally substituted 3-10 membered heterocyclyl having 1-4 heteroatoms, or optionally substituted oxetanyl.

52. The compound of any one of claims 1-48, wherein:
R^{w1} and R^{w2} are taken together with their intervening atoms to form an optionally substituted 5-14 membered ring having 0-6 heteroatoms in addition to the intervening atoms; or
R^{w1} and R^{w2} are taken together with their intervening atoms to form an optionally substituted 5-14 membered partially unsaturated ring having 0-6 heteroatoms in addition to the intervening atoms.

53. The compound of any one of claims 1-48, wherein —C(═NR^{w1})N(R^{w2})— is optionally substituted 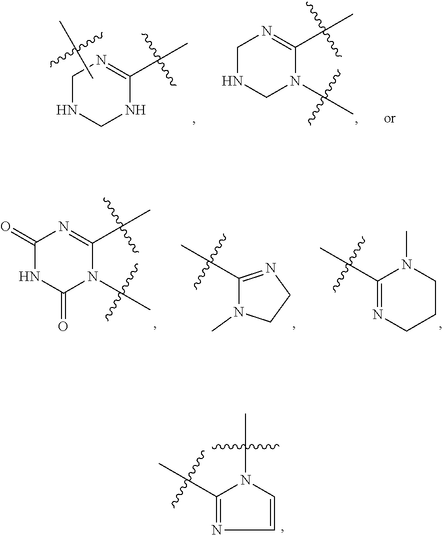
wherein y is independently 0-4, 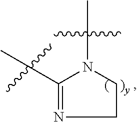
wherein y is independently 0, 1, 2, 3 or 4, 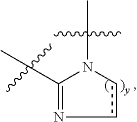
wherein y is independently 0, 1, 2, 3 or 4, and [symbol] is single bond or double bond.

54. The compound of any one of the preceding embodiments, wherein R^{w3} is -L-R′.

55. The compound of any one of the preceding embodiments, wherein R^{w3} is H, optionally substituted C₁₋₁₀ aliphatic, optionally substituted C₁₋₁₀ alkyl, optionally substituted C₁₋₆ haloalkyl, methyl, ethyl, isopropyl, optionally substituted C₃₋₁₀ cycloalkyl, cyclopropyl, cyclohexyl, optionally substituted adamantly, 1-adamantyl, —CH₂—CF₃, optionally substituted C₁₋₁₀ heteroaliphatic having 1-3 heteroatoms, optionally substituted C₆₋₁₀ aryl, optionally substituted phenyl, optionally substituted 5-10 membered heteroaryl having 1-4 heteroatoms, optionally substituted 3-10 membered heterocyclyl having 1-4 heteroatoms, optionally substituted 3-10 membered saturated ring having 0-5 heteroatoms, optionally substituted 3-10 membered partially saturated ring having 0-5 heteroatoms, optionally substituted phenyl, optionally substituted 5-6 membered heteroaryl, 2-pyridyl, 3-pyridyl, or 4-pyridyl.

56. The compound of any one of claims 1-50, wherein:
R^{w2} and R^{w3} are taken together with the nitrogen atom to which they are attached to form an optionally substituted 3-14 membered ring having 0-6 heteroatoms in addition to the nitrogen atom;
R^{w2} and R^{w3} are taken together with the nitrogen atom to which they are attached to form an optionally substituted 3-10 membered aromatic ring having 0-5 heteroatoms in addition to the nitrogen atom; or
R^{w2} and R^{w3} are taken together with the nitrogen atom to which they are attached to form an optionally substituted 3-8 membered monocyclic, non-aromatic ring having 0-3 heteroatoms in addition to the nitrogen atom.

57. The compound of any one of claims 1-50, wherein:
—N(R^{w2})(R^{w3}) is optionally substituted 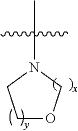
wherein each of x and y is independently 0-4; or
—N(R^{w2})(R^{w3}) is optionally substituted 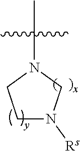
wherein each of x and y is independently 0-4; or
—N(R^{w2})(R^{w3}) is optionally substituted 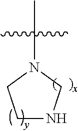
wherein each of x and y is independently 0-4; or
—N(R^{w2})(R^{w3}) is optionally substituted 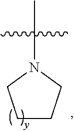
wherein y 0-4; or
—N(R^{w2})(R^{w3}) is optionally substituted 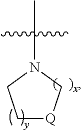
wherein each of x and y is independently 0-4, Q is optionally substituted S, Si, S(O)₂.

58. The compound of any one of claims 1-50, wherein —N(R^{w2})(R^{w3}) is —NH₂,

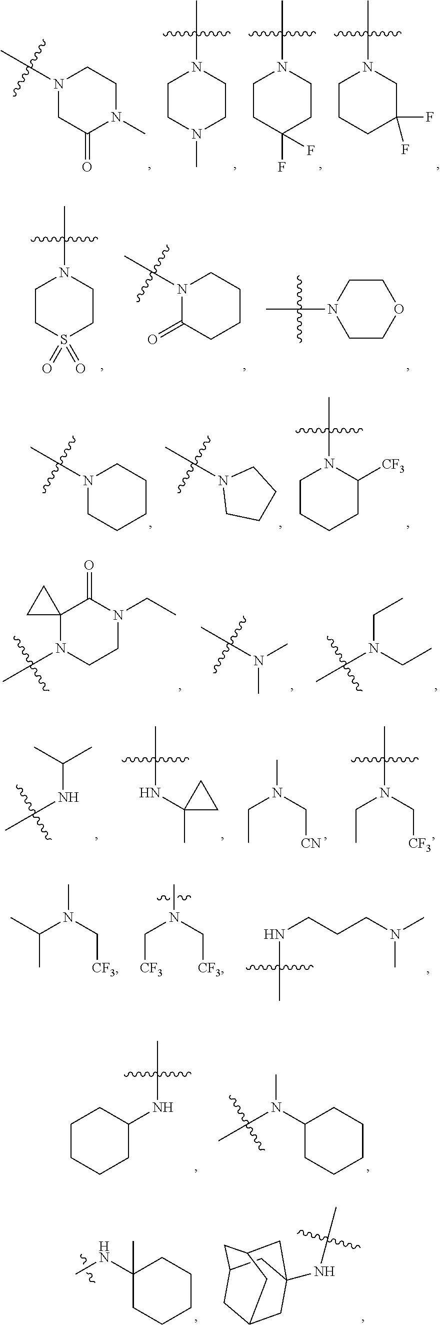

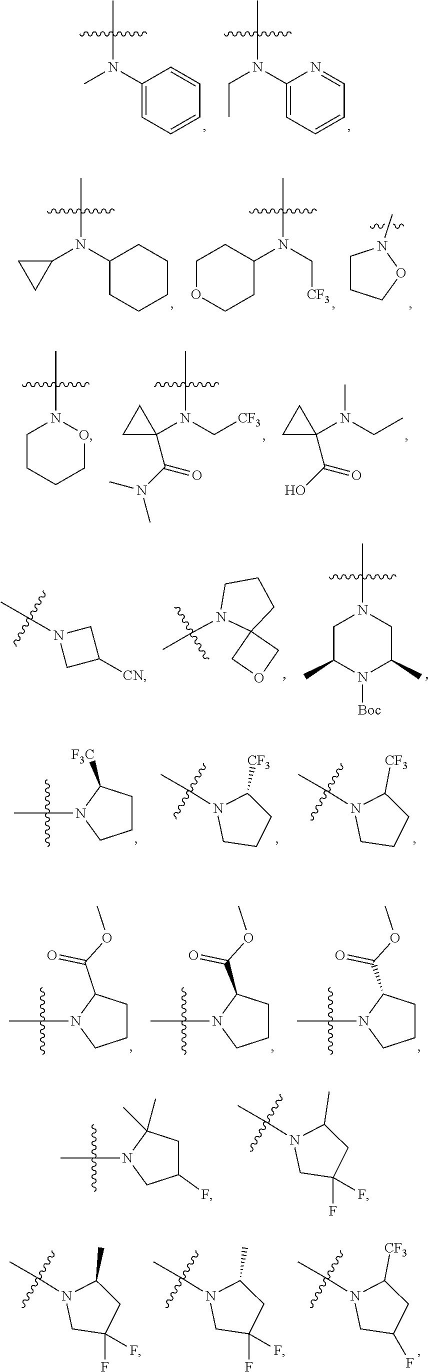

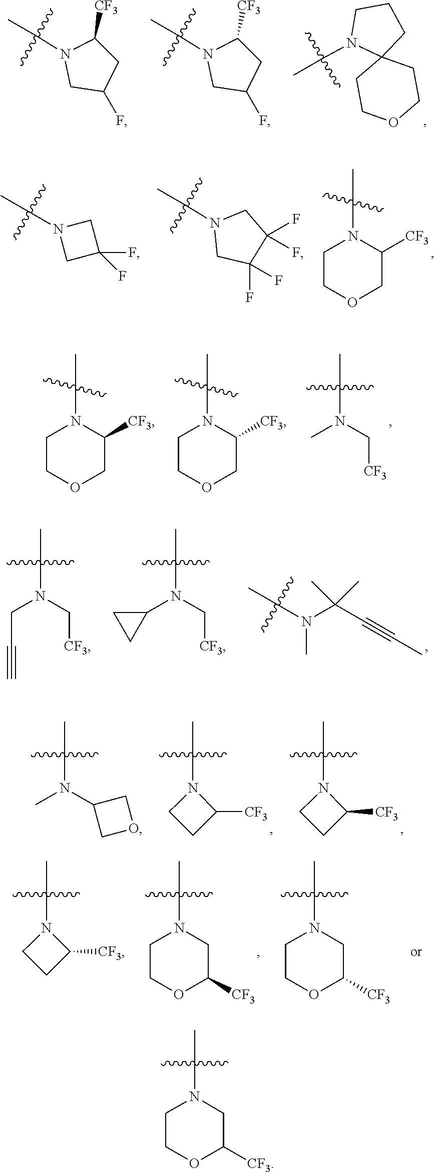

59. The compound of any one of claims 1-43, wherein R⁴ is NH

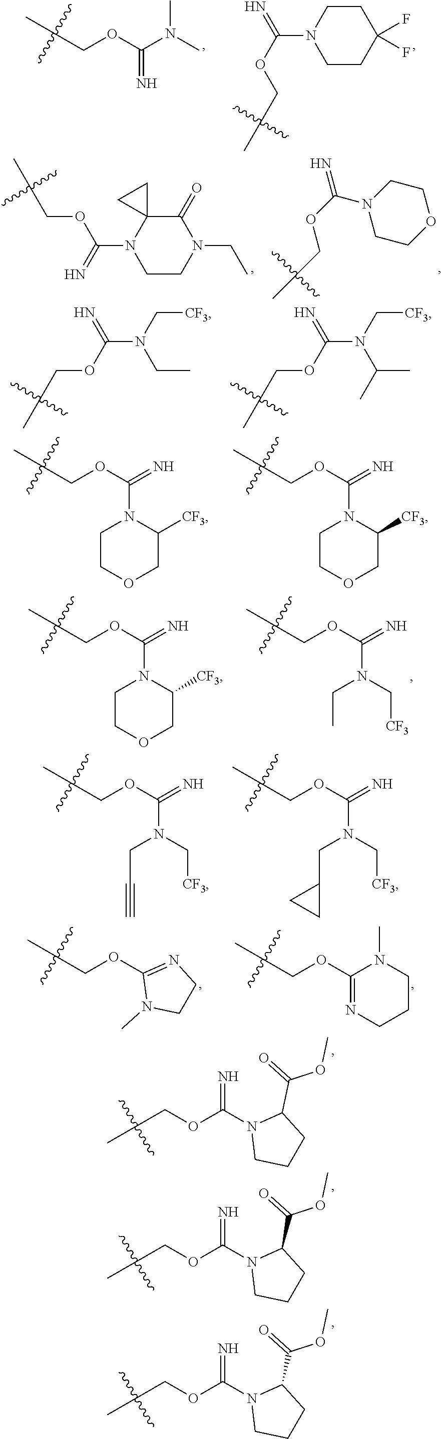

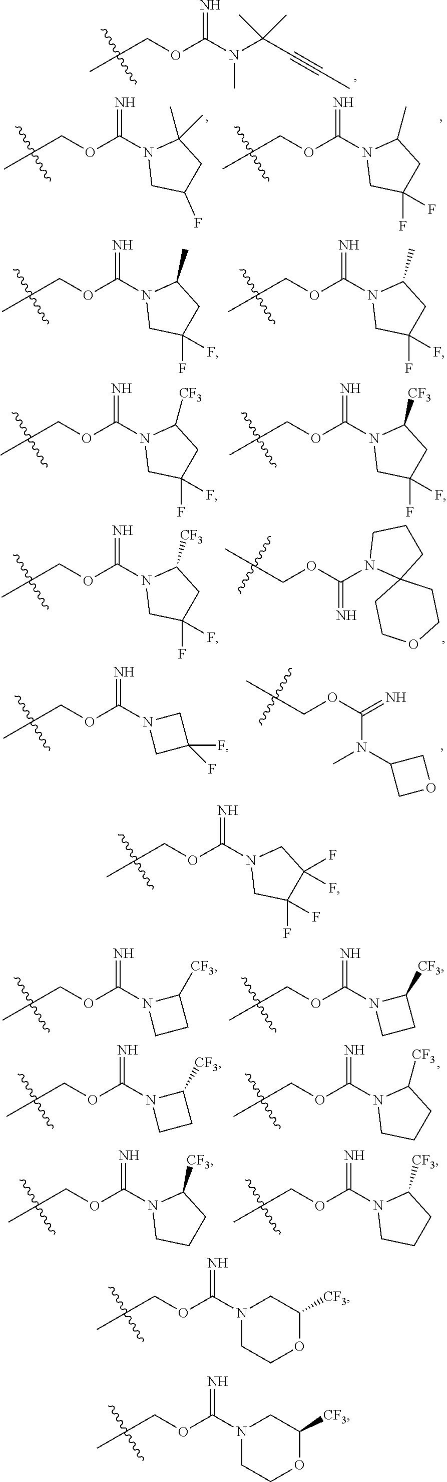

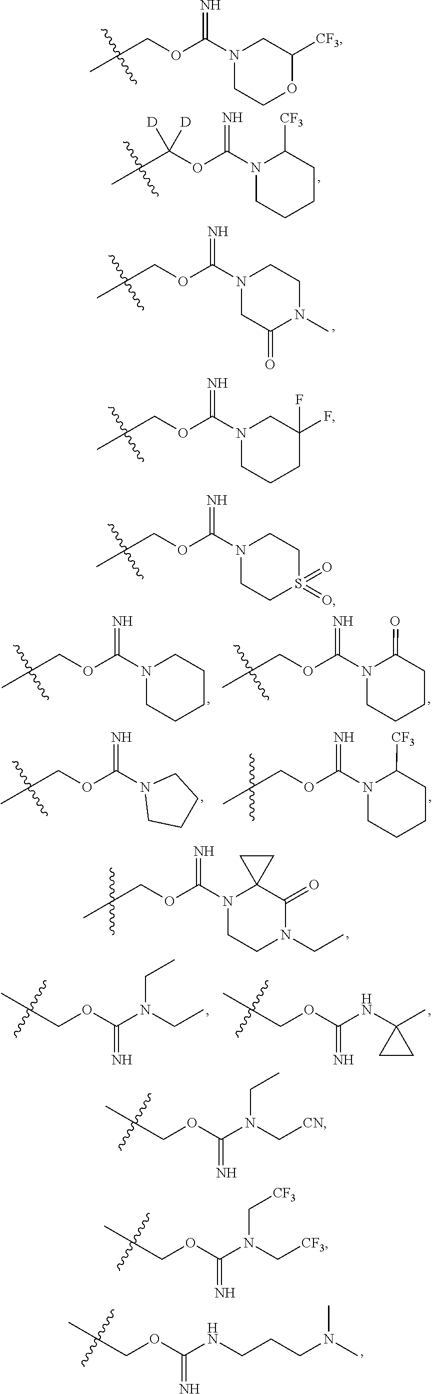

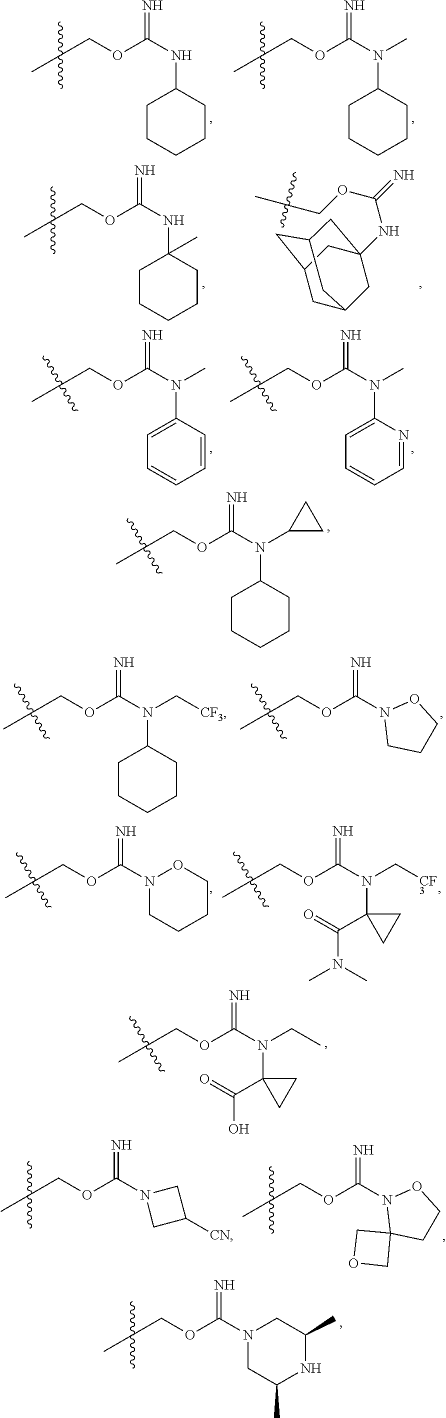

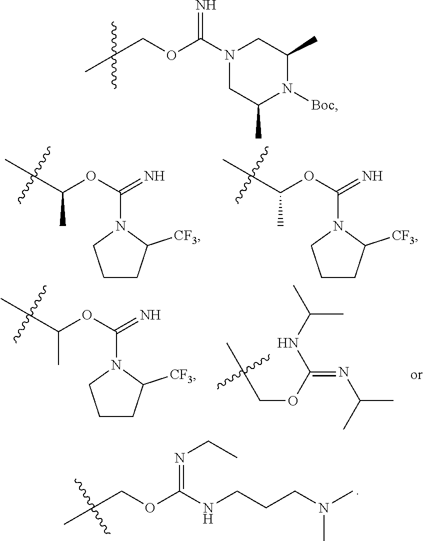

60. A pharmaceutical composition comprising or delivering a compound of any one of the preceding claims or a pharmaceutically acceptable salt thereof, and a pharmaceutically acceptable carrier.

61. A method for inhibiting an activity of a polypeptide comprising a nucleophilic moiety, comprising contacting the polypeptide with a compound or composition of any one of the preceding claims; preferably:
wherein the nucleophilic moiety is —SH or —SeH or a salt form thereof; or
wherein the polypeptide is GPX4;
or
a method for inhibiting cell proliferation in a system, comprising administering to the system a compound or composition of any one of the preceding claims; or
a method for inducing cell death in a system, comprising administering to the system a compound or composition of any one of the preceding claims; or
a method for inducing ferroptosis in a system, comprising administering to the system a compound or composition of any one of the preceding claims.

62. A method for preventing a condition, disorder or disease, comprising administering or delivering to a subject susceptible thereto an effective amount of a compound or composition of any one of the preceding claims; or
a method for preventing a condition, disorder or disease associated with a polypeptide comprising —SeH or a salt form, comprising administering or delivering to a subject susceptible thereto an effective amount of a compound or composition of any one of the preceding claims; or
a method for treating a condition, disorder or disease associated with a polypeptide comprising —SeH or a salt form, comprising administering or delivering to a subject suffering therefrom an effective amount of a compound or composition of any one of the preceding claims.

63. A method for treating a condition, disorder or disease, comprising administering or delivering to a subject suffering therefrom an effective amount of a compound or composition of any one of the preceding embodiments.

64. The method of claim 62 or 63, wherein the condition, disorder or disease is cancer.

65. The method of claim 64, wherein:
the cancer is adrenocortical cancer, anal cancer, biliary cancer, bladder cancer, bone cancer, brain cancer, breast cancer, cervical cancer, colon cancer, endometrial cancer, esophageal cancer, head and neck cancer, intestinal cancer, liver cancer, lung cancer, oral cancer, ovarian cancer, pancreatic cancer, renal cancer, prostate cancer, salivary gland cancer, skin cancer, stomach cancer, testicular cancer, throat cancer, thyroid cancer, uterine cancer, vaginal cancer, sarcoma, or a soft tissue carcinoma; or
wherein the cancer is osteosarcoma, glioma, astrocytoma, neuroblastoma, cancer of the small intestine, bronchial cancer, small cell lung cancer, non-small cell lung cancer, basal cell carcinoma, or melanoma.

66. The method of claim 65, wherein the cancer is the hematologic cancer.

67. The method of claim 66, wherein the hematologic cancer is acute lymphoblastic leukemia (ALL), acute myeloid leukemia (AML), lymphoma (e.g., Hodgkin's lymphoma, NonHodgkin's lymphoma, Burkitt's lymphoma), chronic lymphocytic leukemia (CLL), chronic myelogenous leukemia (CML), Hairy Cell chronic myelogenous leukemia (CML), or multiple myeloma.

68. A compound comprising an isourea moiety or a salt thereof.

69. A compound comprising an isothiourea moiety or a salt thereof.

70. The compound of any one of the preceding claims, wherein:
the compound inhibits an activity of a polypeptide comprising a selenocysteine residue;
compound reacts with selenocysteine or a salt thereof, or a selenocysteine residue in a polypeptide;
—SeH of a selenocysteine or a salt form thereof replaces the isourea moiety;
the compound is capable of inhibiting or preventing cell growth or proliferation;
the compound is capable of inducing cell death;
the compound is capable of inducing ferroptosis;
the effect of the compound is reduced in the presence of a ferroptosis inhibitor;
an observed IC50 for the compound for a capability in the presence of a ferroptosis inhibitor is about or at least about 2-5000, e.g., about or at least about 10-1000, 50-1000, 100-1000, 2, 3, 4, 5, 6, 7, 8, 9, 10, 15, 20, 25, 30, 40, 50, 60, 70, 80, 90, 100, 200, 500 or 1000 fold of that absence of a ferroptosis inhibitor;
an observed IC50 for the compound for a capability in the presence of a ferroptosis inhibitor is about or at least about 10 fold of that absence of a ferroptosis inhibitor;
an observed IC50 for the compound for a capability in the presence of a ferroptosis inhibitor is about or at least about 20 fold of that absence of a ferroptosis inhibitor;
an observed IC50 for the compound for a capability in the presence of a ferroptosis inhibitor is about or at least about 50 fold of that absence of a ferroptosis inhibitor;
an observed IC50 for the compound for a capability in the presence of a ferroptosis inhibitor is about or at least about 100 fold of that absence of a ferroptosis inhibitor;
an observed IC50 for the compound for a capability in the presence of a ferroptosis inhibitor is about or at least about 1-100, e.g., about or at least about 1, 2, 3, 4, 5, 6, 7, 8, 9, 10, 15, 20, 25, 30, 40, 50, 60, 70, 80, 90 or 100 uM;
the concentration of the ferroptosis inhibitor is about or at least about EC50 of the ferroptosis inhibitor when the ferroptosis inhibitor is assessed by itself;
the concentration of the ferroptosis inhibitor is about or at least about 0.1-100, e.g., about or at least about 1-20, 2-10, 0.1, 0.2, 0.3, 0.4, 0.5, 0.6, 0.7, 0.8, 0.9, 1, 1.5, 2, 3, 4, 5, 6, 7, 8, 9, 10, 20, 30, 40, or 50 uM;
the concentration of the ferroptosis inhibitor is about or at least about 1 uM;
the concentration of the ferroptosis inhibitor is about or at least about 2 uM;
the compound inhibits an activity of GPX4;
the compound binds to GPX4; or
wherein the isourea or isothiourea moiety has the structure of R^{w}, wherein R^{w} is -T-C(═NR^{w1})N(R^{w2})(R^{w3}) or a salt thereof, wherein each of R^{w1}, R^{w2} and R^{w3} is independently -L-R′ or a detectable moiety;
T is O or S;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R′ is independently R, —OR, —C(O)R, —C(O)OR, —C(O)N(R)₂, —S(O)R, or —S(O)OR; and
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

71. A compound comprising R^{w}, wherein R^{w} is -T-C(═NR^{w1})N(R^{w2})(R^{w3}) or a salt form thereof, wherein each of R^{w1}, R^{w2} and R^{w3} is independently -L-R′ or a detectable moiety;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
T is O or S;
each R′ is independently R, —OR, —C(O)R, —C(O)OR, —C(O)N(R)₂, —S(O)R, or —S(O)OR; and
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms; or
a compound comprising R^{w}, wherein R″ is —OC(=NR^{w1})N(R^{w2})(R^{w3}) or a salt thereof, wherein each of R^{w1}, R^{w2} and R^{w3} is independently -L-R′ or a detectable moiety;
each L is independently a covalent bond, or an optionally substituted bivalent C₁₋₁₀ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R′ is independently R, —OR, —C(O)R, —C(O)OR, —C(O)N(R)₂, —S(O)R, or —S(O)OR; and
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

72. The compound of claim 71 or 72, comprising -L^{w}-R^{w}, wherein LV is a covalent bond, or an optionally substituted bivalent C₁₋₆ aliphatic or heteroaliphatic having 1-6 heteroatoms, wherein one or more methylene unit is optionally and independently replaced with —C(R′)₂—, -Cy-, —O—, —S—, —N(R′)—, —C(O)—, —C(S)—, —C(NR′)—, —C(O)N(R′)—, —C(S)N(R′)—, —C(NR′)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted, 3-20 membered, monocyclic, bicyclic or polycyclic ring having 0-10 heteroatoms;
each R′ is independently R, —OR, —C(O)R, —C(O)OR, —C(O)N(R)₂, —S(O)R, or —S(O)OR; and
each R is independently —H, or an optionally substituted group selected from C₁₋₂₀ aliphatic, C₁₋₂₀ heteroaliphatic having 1-10 heteroatoms, C₆₋₂₀ aryl, 5-20 membered heteroaryl having 1-10 heteroatoms, 3-20 membered heterocyclyl having 1-10 heteroatoms and combinations thereof, wherein each combination independently has 1-30 carbon atoms and 0-10 heteroatoms; or:
two R groups are optionally and independently taken together to form a covalent bond, or:
two or more R groups on the same atom are optionally and independently taken together with the atom to form an optionally substituted, 3-20 membered, monocyclic, bicyclic or polycyclic ring having, in addition to the atom, 0-10 heteroatoms; or:
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms.

73. The compound of any one of claims 73-75, wherein the compound comprises —C(═O)-L^{w}-R^{w}.

74. A compound of Table C1 or C2, or a pharmaceutically acceptable salt thereof.
